(19) United States
(12) Patent Application Publication
(10) Pub. No.: US 2023/0173060 A1
(43) Pub. Date: Jun. 8, 2023

(54) LARGE SEQUENCE PAN-CORONAVIRUS VACCINE COMPOSITIONS

(71) Applicant: THE REGENTS OF THE UNIVERSITY OF CALIFORNIA, Oakland, CA (US)

(72) Inventor: Lbachir BenMohamed, Irvine, CA (US)

(21) Appl. No.: 18/046,875

(22) Filed: Oct. 14, 2022

Related U.S. Application Data
(63) Continuation-in-part of application No. PCT/US21/27355, filed on Apr. 14, 2021.
(60) Provisional application No. 63/084,421, filed on Sep. 28, 2020.
(60) Provisional application No. 63/009,907, filed on Apr. 14, 2020.
(60) Provisional application No. 63/349,799, filed on Jun. 7, 2022.
(60) Provisional application No. 63/349,904, filed on Jun. 7, 2022.
(60) Provisional application No. 63/302,454, filed on Jan. 24, 2022.

(51) Int. Cl. A61K 39/215; A61K 39/39; A61P 31/14

(52) U.S. Cl. CPC A61K 39/215; A61K 39/39; A61P 31/14; A61K 2039/70

(57) ABSTRACT

Pan-coronavirus vaccines for inducing efficient, powerful and long-lasting protection against all Coronaviruses infections and diseases, comprising multiple highly conserved large sequences which may comprise one or more conserved B, CD4 and CD8 T cell epitopes that help provide multiple targets for the body to develop an immune response for preventing a Coronavirus infection and/or disease. In certain embodiments, the large sequences are conserved proteins or large sequences, e.g., sequences that are highly conserved among human coronaviruses and/or animal coronaviruses (e.g., coronaviruses isolated from animals susceptible to coronavirus infections).

### CROSS-REFERENCES TO RELATED APPLICATIONS

This application is a continuation in part and claims benefit of PCT Application No. PCT/US20211027355 filed Apr. 14, 2021, which claims benefit of U.S. Provisional Application No. 63/084,421 filed Sep. 28, 2020, and U.S. Provisional Application No. 63/009,907 filed Apr. 14, 2020, the specifications of which are incorporated herein in their entirety by reference.

This application is a non-provisional and claims benefit of U.S. Provisional Application No. 63/349,799 filed Jun. 7, 2022, U.S. Provisional Application No. 63/349,904 filed Jun. 7, 2022, and U.S. Provisional Application No. 63/302,454 filed Jan. 24, 2022, the specifications of which are incorporated herein in their entirety by reference.

### STATEMENT REGARDING FEDERALLY SPONSORED RESEARCH OR DEVELOPMENT

This invention was made with government support under Grant No. AI158060, AI150091, AI143348, AI147499, AI143326, AI138764, AI124911 and AI110902 awarded by National institutes of Allergy and infectious Diseases. The government has certain rights in the invention.

### FIELD OF THE INVENTION

The present invention relates to vaccines, for example viral vaccines, such as those directed to coronaviruses, e.g., pan-coronavirus vaccines.

### BACKGROUND OF THE INVENTION

Over the last two decades, there have been three deadly human outbreaks of Coronaviruses (CoVs) caused by emerging zoonotic CoVs: SARS-CoV, MERS-CoV, and the latest highly transmissible and deadly SARS-CoV-2, which has caused the current COVID-19 global pandemic. All three deadly CoVs originated from bats, the natural hosts, and transmitted to humans via various intermediate animal reservoirs (e.g., pangolins, civet cats and camels). Because there is currently no universal pan-Coronavirus vaccine available, it remains highly possible that other global COVID-like pandemics will emerge in the coming years, caused by yet another spillover of an unknown zoonotic bat-derived SARS-like Coronavirus (SL-CoV) into an unvaccinated human population.

Neutralizing antibodies and antiviral effector CD4⁺ and CD8⁺ T cells appear to be crucial in reducing viral load in the majority of infected asymptomatic and convalescent patients. However, very little information exists on the antigenic landscape and the repertoire of B-cell and CD4⁺ and CD8⁺ T cell epitopes that are conserved among human and bat Coronavirus strains.

### SUMMARY OF THE INVENTION

Current vaccines on the market primarily are directed to the spike antigen only and focus on induction of antibody responses. However, this strategy excludes other more conserved antigens of the virus and misses the opportunity to target long-term cross-reactive memory T cell responses. The present invention aims to create a vaccine that not only includes the spike antigen but also includes other conserved antigens of the virus to induce an immune response with both antibodies and T cells.

Determining the antigen and epitope landscapes that are antigenic, immunogenic, protective and conserved among human and animal Coronaviruses as well as the repertoire, phenotype and function of B cells and CD4+ and CD8+ T cells that correlate with resistance seen in asymptomatic COVID-19 patients may inform in the development of future pan-Coronavirus vaccines. The present invention describes using several immuno-informatics and sequence alignment approaches and several immunological assays both in vitro in humans and in vivo in animal models (e.g. mice, hamster and monkeys) to identify several antigenic, immunogenic, protective highly conserved large sequences that include human B cell, CD4+ and CD8+ T cell epitopes that are highly conserved, e.g., highly conserved in: (i) greater than 81,000 SARS-CoV-2 human strains identified in 190 countries on six continents: (ii) six circulating CoVs that caused previous human outbreaks of the “Common Cold”; (iii) nine SL-CoVs isolated from bats; (iv) nine SL-CoV isolated from pangolins; (v) three SL-CoVs isolated from civet cats; and (vi) four MERS strains isolated from camels. Furthermore, the present invention describes the identification of cross-reactive epitopes that: recalled B cell, CD4+ and CD8+ T cells from both COVID-19 patients and healthy individuals who were never exposed to SARS-CoV-2; and induced strong B cell and T cell responses in “humanized” Human Leukocyte Antigen (HLA)-DR1/HLA-A*02:01 double transgenic mice as well as in humans that do not express HLA-DR-1 or HLA-A*02:01 haplotypes. Unlike small epitopes that are restricted to certain HLA haplotype, the large sequences encompass several epitopes restricted to large numbers of HLA haplotypes, thus ascertaining large vaccine coverage of human population regardless of HLA haplotypes and regardless of race and ethnicity.

The present invention is not limited to vaccine compositions for use in humans. The present invention includes vaccine compositions for use in other pet animals such as dogs, cats, etc.

The vaccine compositions herein have the potential to provide lasting B and T cell immunity regardless of Coronaviruses mutations. This may be due at least partly because the vaccine compositions target highly conserved structural and non-structural Coronavirus antigens, such as Coronavirus nucleoprotein (also known as nucleocapsid), in combination with other Coronavirus structural and non-structural antigens with a low mutation rate found in perhaps every human and animal Coronaviruses variants and strains.

The present invention is also related to selecting highly conserved structural (e.g., spike protein) and non-structural Coronavirus antigens inside the virus (e.g., non-spike protein such as nucleocapsid), which may be viral proteins that are normally not necessarily under mutation pressure by the immune system.

The present invention provides pan-Coronavirus recombinant vaccine compositions that induces board, strong and long-lasting B and T cell protective immune responses in humans and pets and animals.

In certain embodiments, the vaccine compositions are for use in humans. In certain embodiments, the vaccine compositions are for use in animals, such as but not limited to mice, cats, dogs, non-human primates, other animals susceptible to coronavirus infection, other animals that may function as preclinical animal models for coronavirus infections, etc.

As used herein, the term “multi-epitope” refers to a composition comprising more than one B and T cell epitope wherein at least: one CD4 and/or CD8 T cell epitope is MHC-restricted and recognized by a TCR, and at least one epitope is a B cell epitope. For example, the vaccine compositions herein may be multi-epitope pan-coronavirus vaccine compositions.

As used herein, the term “recombinant vaccine composition” may refer to one or more proteins or peptides encoded by one or more recombinant genes, e.g. genes that have been cloned into one or more systems that support the expression of said gene(s). The term “recombinant vaccine composition” may refer to the recombinant genes or the system that supports the expression of said recombinant genes.

For example, the present invention provides a pan-coronavirus recombinant vaccine composition comprising one or more large sequences, wherein each of the one or more large sequences comprise at least one of: one or more conserved coronavirus B-cell target epitopes; one or more conserved coronavirus CD4+ T cell target epitopes; and/or one or more conserved coronavirus CD8+ T cell target epitopes; wherein at least one epitope is derived from a non-spike protein.

The present invention also features a pan-coronavirus recombinant vaccine composition, the composition comprising two or more large sequences, wherein each of the two or more large sequences comprise at least one of: one or more conserved coronavirus B-cell target epitopes; one or more conserved coronavirus CD4+ T cell target epitopes; and/or one or more conserved coronavirus CD8+ T cell target epitopes; wherein at least one epitope is derived from a non-spike protein.

The present invention also features a pan-coronavirus recombinant vaccine composition, the composition comprising whole spike protein; and one or both of: one or more conserved coronavirus CD4+ T cell target epitopes; and/or one or more conserved coronavirus CD8+ T cell target epitopes; wherein at least one epitope is derived from a non-spike protein.

The present invention also features a pan-coronavirus recombinant vaccine composition, the composition comprising at least a portion of spike protein, the portion of spike protein comprising a trimerized SARS-CoV-2 receptor-binding domain (RBD); and one or both of: one or more conserved coronavirus CD4+ T cell target epitopes; one or more conserved coronavirus CD8+ T cell target epitopes; wherein at least one epitope is derived from a non-spike protein.

The present invention also features a pan-coronavirus recombinant vaccine composition, the composition comprising whole spike protein; and one or more conserved coronavirus CD4+ T cell target epitopes; and one or more conserved coronavirus CD8+ T cell target epitopes; wherein at least one epitope is derived from a non-spike protein.

The present invention also features a pan-coronavirus recombinant vaccine composition, the composition comprising at least a portion of spike protein, the portion of spike protein comprising a trimerized SARS-CoV-2 receptor-binding domain (RBD); and one or more conserved coronavirus CD4+ T cell target epitopes; and one or more conserved coronavirus CD8+ T cell target epitopes; wherein at least one epitope is derived from a non-spike protein.

The present invention also features a pan-coronavirus recombinant vaccine composition, the composition comprising an antigen delivery system encoding one or more large sequences, wherein each of the one or more large sequences comprise at least one of: one or more conserved coronavirus B-cell target epitopes; one or more conserved coronavirus CD4+ T cell target epitopes: and/or one or more conserved coronavirus CD8+ T cell target epitopes; wherein at least one epitope is from a non-spike protein.

The present invention also features a pan-coronavirus recombinant vaccine composition, the composition comprising an antigen delivery system encoding two or more large sequences, wherein each of the two or more large sequences comprise at least one of: one or more conserved coronavirus B-cell target epitopes; one or more conserved coronavirus CD4+ T cell target epitopes; and/or one or more conserved coronavirus CD8+ T cell target epitopes; wherein at least one epitope is derived from a non-spike protein.

The present invention also features a pan-coronavirus recombinant vaccine composition, the composition comprising an antigen delivery system encoding whole spike protein, and one or both of: one or more conserved coronavirus CD4+ T cell target epitopes; and/or one or more conserved coronavirus CD8+ T cell target epitopes; wherein at least one epitope is derived from a non-spike protein.

The present invention also features a pan-coronavirus recombinant vaccine composition, the composition comprising an antigen delivery system encoding at least a portion of spike protein, the portion of spike protein comprising a trimerized SARS-CoV-2 receptor-binding domain (RBD); and one or both of: one or more conserved coronavirus CD4+ T cell target epitopes; one or more conserved coronavirus CD8+ T cell target epitopes; wherein at least one epitope is derived from a non-spike protein.

The present invention also features a pan-coronavirus recombinant vaccine composition, the composition comprising an antigen delivery system encoding whole spike protein; and one or more conserved coronavirus CD4+ T cell target epitopes; and one or more conserved coronavirus CD8+ T cell target epitopes; wherein at least one epitope is derived from a non-spike protein.

The present invention also features a pan-coronavirus recombinant vaccine composition, the composition comprising an antigen delivery system encoding at least a portion of spike protein, the portion of spike protein comprising a trimerized SARS-CoV-2 receptor-binding domain (RBD); and one or more conserved coronavirus CD4+ T cell target epitopes, and one or more conserved coronavirus CD8+ T cell target epitopes; wherein at least one epitope is derived from a non-spike protein.

Referring to the aforementioned compositions and the embodiments herein, in some embodiments, the non-spike protein is ORF1ab protein, ORF3a protein, Envelope protein, Membrane glycoprotein, ORF6 protein, ORF7a protein, ORF7b protein, ORF8 protein, Nucleocapsid protein and ORF10 protein.

In some embodiments, the one or more large sequences are highly conserved among human and animal coronaviruses. In some embodiments, the one or more large sequences are derived from at least one of SARS-CoV-2 protein. In some embodiments, the one or more large sequences are derived from one or more of: one or more SARS-CoV-2 human strains or variants in current circulation; one or more coronaviruses that has caused a previous human outbreak; one or more coronaviruses isolated from animals selected from a group consisting of bats, pangolins, civet cats, minks, camels, and other animal receptive to coronaviruses; or one or more coronaviruses that cause the common cold. In some embodiments, the one or more SARS-CoV-2 human strains or variants in current circulation are selected from: variant B.1.177; variant B.1.160, variant B.1.1.7 (UK), variant P.1 (Japan % Brazil), variant B.1.351 (South Africa), variant B.1.427 (California), variant B.1.429 (California), variant B.1.258; variant 8.1.221; variant B.1.367; variant 8.1.1.277; variant 8.1.1.302; variant B.1.525; variant B.1.526, variant S:677H; variant S:677P; B.1.617.2-Delta, variant B.1.1.529-Omicron (BA.1); sub-variant Omicron (BA.1); sub-variant Omicron (BA.2); sub-variant Omicron (BA.3); sub-variant Omicron (BA.4); sub-variant Omicron (BA.5). In some embodiments, the one or more coronaviruses that cause the common cold are selected from: 229E alpha coronavirus, NL63 alpha coronavirus, OC43 beta coronavirus, and HKU1 beta coronavirus. In some embodiments, the conserved large sequences are selected from Variants Of Concern or Variants Of Interest.

In some embodiments, the composition comprises two or more large sequences. In some embodiments, the composition comprises three or more large sequences. In some embodiments, the composition comprises two large sequences. In some embodiments, the composition comprises three large sequences. In some embodiments, the composition comprises four large sequences. In some embodiments, the composition comprises five large sequences.

In some embodiments, the large sequences are derived from structural proteins, non-structural proteins, or a combination thereof. In some embodiments, the large sequences or target epitopes are derived from a SARS-CoV-2 protein selected from a group consisting of: ORF1ab protein, Spike glycoprotein, ORF3a protein, Envelope protein, Membrane glycoprotein, ORF6 protein, ORF7a protein, ORF7b protein, ORF8 protein, Nucleocapsid protein an ORF10 protein.

In some embodiments, the large sequence or the target epitope derived from the Spike glycoprotein is RBD. In some embodiments, the large sequence or the target epitope derived from the Spike glycoprotein is NTD. In some embodiments, the large sequence or the target epitope derived from the Spike glycoprotein includes both the RBD and NTD regions. In some embodiments, the large sequence or the target epitope derived from the spike glycoprotein are recognized by neutralizing and blocking antibodies.

In some embodiments, the large sequence or the target epitope derived from the spike glycoprotein induces neutralizing and blocking antibodies. In some embodiments, the large sequence or the target epitope derived from the spike glycoprotein induces neutralizing and blocking antibodies that recognize and neutralize the virus.

In some embodiments, the large sequence or the target epitope derived from the spike glycoprotein induces neutralizing and blocking antibodies that recognize the spike protein.

In some embodiments, the ORF1ab protein comprises nonstructural protein (Nsp) 1, Nsp2, Nsp3, Nsp4, Nsp5, Nsp6, Nsp7, Nsp3, Nsp9, Nsp10, Nsp11, Nsp12, Nsp13, Nsp14, Nsp15 and Nsp16. In some embodiments, the one or more conserved coronavirus CD8+ T cell target epitopes are selected from: spike glycoprotein, Envelope protein, ORF1ab protein, ORF7a protein, ORF8a protein, ORF10 protein, or a combination thereof. In some embodiments, the one or more conserved coronavirus CD8+ T cell target epitopes are selected from: S₂₋₁₀, S₁₂₂₅₋₁₂₂₈, S₁₀₀₀₋₁₀₀₈, S₉₅₈₋₉₆₆, E₂₀₋₂₈, ORF1ab₁₆₇₈₋₁₆₈₃, ORF1ab₂₃₆₃₋₂₃₇₁, ORF1ab₃₀₁₃₋₃₀₂₁, ORF1ab₃₁₈₃₋₃₁₉₁, ORF1ab₅₄₇₀₋₅₄₇₈, ORF1ab₆₇₄₉₋₆₇₅₇, ORF7b₂₆₋₃₄, ORF8a₇₃₋₈₁, ORF1₀₃₋₁₁, and ORF1s-13. In some embodiments, the one or more conserved coronavirus CD8+ T cell target epitopes are selected from SEQ ID NO: 2-29 or SEQ ID NO: 194-213. In some embodiments, the one or more conserved coronavirus CD8+ T cell target epitopes are selected from SEQ ID NO: 30-57 or SEQ ID NO: 214-234. In some embodiments, the one or more conserved coronavirus CD4+ T cell target epitopes are selected from: spike glycoprotein, Envelope protein, Membrane protein, Nucleocapsid protein, ORF1a protein, ORF1ab protein, ORF6 protein, ORF7a protein, ORF7b protein, ORF8 protein, or a combination thereof. In some embodiments, the one or more conserved coronavirus CD4-+ T cell target epitopes are selected from: ORF1a₁₃₅₀₋₁₃₆₅, ORF1ab₅₀₁₉₋₅₀₃₃, ORF6₁₂₋₂₈, ORF1ab₆₀₈₈₋₆₁₀₂, ORF1ab₆₄₂₀₋₆₄₃₄, ORF1a₁₈₀₁₋₁₈₁₅, S₁₋₁₃, E₂₆₋₄₀, E₂₀₋₃₄, M₁₇₈₋₁₉₀, N₃₈₈₋₄₀₃, ORF7a₃₋₁₇, ORF7a₁₋₁₅, ORF7b₈₋₂₂, ORF7a₉₈₋₁₁₂, and ORF8₁₋₁₅. In some embodiments, the one or more conserved coronavirus CD4+ T cell target epitopes are selected from SEQ ID NO: 58-73 or SEQ ID NO: 235-253. In some embodiments, the one or more conserved coronavirus CD4+ T cell target epitopes are selected from SEQ ID NO: 74-105 or SEQ ID NO: 254-272. In some embodiments, the one or more conserved coronavirus B cell target epitopes are selected from Spike glycoprotein. In some embodiments, the one or more conserved coronavirus B cell target epitopes are selected from: S₂₈₇₋₃₁₇, S₅₂₄₋₅₉₈, S₆₀₁₋₄₄₀, S₈₀₂₋₈₁₉, S₈₈₃₋₉₀₉, S₃₆₉₋₃₉₃, S₄₄₀₋₅₀₁, S₁₁₃₃₋₁₁₇₂, S₃₂₉₋₃₆₃, and S₁₃₋₃₇. In some embodiments, the one or more coronavirus B cell target epitopes are selected from SEQ ID NO: 106-116 or SEQ ID NO: 273 to 280. In some embodiments, the one or more coronavirus B cell target epitopes are selected from SEQ ID NO: 117-138 or SEQ ID NO: 281-294.

In some embodiments, the one or more conserved coronavirus B cell target epitopes are in the form of a large sequence. In some embodiments, the large sequence is full length spike glycoprotein. In some embodiments, the large sequence is a partial spike glycoprotein. In some embodiments, the spike glycoprotein has two consecutive proline substitutions at amino acid positions 986 and 987. In some embodiments, the spike glycoprotein has single amino acid substitutions at amino acid positions comprising Tyr-83 and Tyr-489, Gln-24 and Asn-487. In some embodiments, the transmembrane anchor of the spike protein has an intact S1-S2 cleavage site. In some embodiments, the spike protein is in its stabilized conformation. In some embodiments, the spike protein is stabilized with proline substitutions at amino acid positions 986 and 987 at the top of the central helix in the S2 subunit.

In some embodiments, the one or more large sequences are derived from a whole protein sequence expressed by SARS-CoV-2. In some embodiments, the one or more large sequences are derived from a partial protein sequence expressed by SARS-CoV-2. In some embodiments, the one or more large conserved sequences from the spike protein is from a full-length spike glycoprotein. In some embodiments, the one or more large conserved sequences from the spike protein is from a partial spike glycoprotein. In some embodiments, the one or more large sequences comprises Spike glycoprotein (5) or a portion thereof, Nucleoprotein or a portion thereof, Membrane protein or a portion thereof, and ORF1a/b or a portion thereof. In some embodiments, the one or more large sequences comprises Spike glycoprotein (S) or a portion thereof, Nucleoprotein or a portion thereof, and ORF1a/b or a portion thereof. In some embodiments, the portion of the Spike glycoprotein is RBD. In some embodiments, the one or more large sequences is selected from the group consisting of: ORF1ab protein, Spike glycoprotein, ORF3a protein, Envelope protein, Membrane glycoprotein, ORF6 protein, ORF7a protein, ORF7b protein, ORF8 protein, Nucleocapsid protein an ORF10 protein. In some embodiments, the ORF1ab protein comprises nonstructural protein (Nsp) 1, Nsp2, Nsp3, Nsp4, Nsp5, Nsp6, Nsp7, Nsp8, Nsp9, Nsp10, Nsp11, Nsp12, Nsp13, Nsp14, Nsp15 and Nsp16. In some embodiments, one or more of the large sequences comprises a T-cell epitope restricted to a large number of human class 1 and class 2 HLA haplotypes and are not restricted to HLA-0201 for class 1 or HLA-DR for class 2.

In some embodiments, the large sequences are derived from structural proteins, non-structural proteins, or a combination thereof.

The present invention also features a recombinant vaccine composition comprising full-length spike protein. The present invention also features a recombinant vaccine composition comprising full-length spike protein or partial spike protein.

In some embodiments, the spike protein comprises Tyr-489 and Asn-487. In some embodiments, Tyr-489 and Asn-487 help with interaction with Tyr 83 and Gln-24 on ACE-2. In some embodiments, the spike protein comprises Gln-493. In some embodiments, Gln-493 helps with interaction with Glu-35 and Lys-31 on ACE-2. In some embodiments, the spike protein comprises Tyr-505. In some embodiments, Tyr-505 helps with interaction with Glu-37 and Arg-393 on ACE-2.

In some embodiments, the composition comprises a trimerized SARS-CoV-2 receptor-binding domain (RBD) sequence. In some embodiments, the trimerized SARS-CoV-2 receptor-binding domain (RBD) sequence is modified by the addition of a T4 fibritin-derived foldon trimerization domain. In some embodiments, the addition of a T4 fibritin-derived foldon trimerization domain increases immunogenicity by multivalent display. In some embodiments, the composition encodes the trimerized SARS-CoV-2 spike glycoprotein RBD antigen together with the one or more highly conserved structural and non-structural SARS-CoV-2 antigens. In some embodiments, the sequence for the antigen is GenBank accession number, MN908947.3. In some embodiments, the conserved large sequences are selected from the Variants Of Concern and Variants Of Interest. In some embodiments, the composition comprises a mutation 682-RRAR-685→682-QQAQ-685 in the S1-S2 cleavage site.

In some embodiments, the composition comprises at least one proline substitution. In some embodiments, the composition comprises at least two proline substitutions. In some embodiments, the proline substitution is at position K986 and V987. In some embodiments, the composition comprises K986P and V987P mutations.

In some embodiments, the large sequences are selected from SEQ ID NO. 182-185 (Table 1) or SEQ ID NO: 148-159 or SEQ ID NO: 348-359 (Table 10).

In some embodiments, the composition further comprises a pharmaceutical carrier. [0044] in some embodiments, the linker comprises T2A. In some embodiments, the linker is selected from T2A, E2A, and P2A. In some embodiments, a different linker is disposed between each open reading frame. [0045] in some embodiments, the vaccine constructs are for humans. In some embodiments, the composition comprises human CXCL-11 and IL-7 or IL-2 or IL-15. In some embodiments, the vaccine constructs are for animals. In some embodiments, the composition comprises animal CXCL-11 and IL-7 or IL-2 or IL-15. In some embodiments, the animals are cats and dogs.

In some embodiments, the delivery system is an adenovirus system. In some embodiments, the adenovirus delivery system is Ad26, Ad5, Ad35, or a combination thereof. In some embodiments, one or more of the large sequences are operatively linked to a generic promoter. In some embodiments, the generic promoter is a CMV or a CAG promoter. In some embodiments, the one or more large sequences are operatively linked to a lung-specific promoter. In some embodiments, the lung-specific promoter is SpB or CD144. In some embodiments, the composition further comprises a T cell attracting chemokine.

In some embodiments, the antigen delivery system further encodes a T cell attracting chemokine.

In some embodiments, the antigen delivery system comprises two delivery systems, wherein a second delivery system encodes the T cell attracting chemokine. In some embodiments, the T cell attracting chemokine is CCL5, CXCL9, CXCL10, CXCL11, or a combination thereof. In some embodiments, the T cell attracting chemokine is operatively linked to a lung-specific promoter. In some embodiments, the T cell attracting chemokine is operatively linked to a generic promoter. In some embodiments, the composition further comprises a composition that promotes T cell proliferation.

In some embodiments, the antigen delivery system further encodes a composition that promotes T cell proliferation. In some embodiments, the antigen delivery system comprises two delivery systems, wherein a second delivery system encodes the composition that promotes T cell proliferation. In some embodiments, the composition that promotes T cell proliferation is IL-7, IL-2, or IL-15. In some embodiments, the composition that promotes T cell proliferation is operatively linked to a lung-specific promoter. In some embodiments, the composition that promotes T cell proliferation is operatively linked to a generic promoter. In some embodiments, the T cell attracting chemokine and the composition that promotes T cell proliferation are driven by the same promoter. In some embodiments, the vaccine further encodes a peptide comprising a T cell attracting chemokine and a composition that promotes T cell proliferation. In some embodiments, the peptide is operatively linked to a lung-specific promoter. In some embodiments, the peptide is operatively linked to a generic promoter. In some embodiments, the lung-specific promoter is SpB or CD144. In some embodiments, the generic promoter is a CMV or a CAG promoter.

In some embodiments, the antigen delivery system further encodes a molecular adjuvant. In some embodiments, the antigen delivery system comprises two delivery systems, wherein a second delivery system encodes the molecular adjuvant. In some embodiments, the molecular adjuvant is CpG. In some embodiments, the molecular adjuvant is a CpG polymer. In some embodiments, the molecular adjuvant is flagellin. In some embodiments, the molecular adjuvant is operatively linked to a promoter. In some embodiments, the promoter is a lung-specific promoter or a generic promoter.

In some embodiments, one or more of the large sequences are separated by a linker. In some embodiments, each of the large sequences are separated by a linker. In some embodiments, the linker is from 2 to 10 amino acids in length.

In some embodiments, the recombinant vaccine composition comprises a tag, e.g., one or more of the large sequences comprises a tag. In some embodiments, the tag is a His tag.

The present invention also includes a rVSV-panCoV recombinant vaccine composition comprising any of the vaccine compositions herein.

The present invention also includes a rAdV-panCoV recombinant vaccine composition comprising any of the vaccine compositions herein.

In some embodiments, the compositions are for use as a vaccine. In some embodiments, the compositions are for use as immunotherapy for the prevention and treatment of Coronaviruses infections and diseases. In some embodiments, the composition is used to prevent a coronavirus disease in a subject.

In some embodiments, the composition is used to prevent a coronavirus infection prophylactically in a subject. In some embodiments, the composition elicits an immune response in a subject. In some embodiments, the composition prolongs an immune response induced by the pan-coronavirus recombinant vaccine composition and increases T-cell migration to the lungs.

The present invention also includes a pan-coronavirus recombinant vaccine composition comprising SEQ ID NO: 139-147 (Table 10).

Non-spike proteins include any of the coronavirus proteins other than spike, such as but not limited to Envelope protein, Membrane protein, Nucleocapsid protein, ORF1a protein, ORF1ab protein, ORF6 protein, ORF7a protein, ORF7b protein, ORF8 protein, etc.

In certain embodiments, the compositions of the present invention, e.g., the large sequences, comprise one or more conserved target epitopes, e.g.; one or more conserved coronavirus B-cell target epitopes; one or more conserved coronavirus CD4+ T cell target epitopes; and/or one or more conserved coronavirus CD8+ T cell target epitopes. In some embodiments, a conserved target epitope is one that is one of the 5 most conserved epitopes (for its epitope type, e.g., B cell, CD4 T cell, CD8 T cell) identified in a sequence alignment and analysis. In some embodiments, a conserved target epitope is one that is one of the 10 most conserved epitopes (for its epitope type, e.g., B cell, CD4 T cell, CD8 T cell) identified in a sequence alignment and analysis. In some embodiments, a conserved target epitope is one that is one of the 15 most conserved epitopes (for its epitope type, e.g., B cell. CD4 T cell, CD8 T cell) identified in a sequence alignment and analysis. In some embodiments, a conserved target epitope is one that is one of the 20 most conserved epitopes (for its epitope type, e.g., B cell, CD4 T cell. CD8 T cell) identified in a sequence alignment and analysis. In some embodiments, a conserved target epitope is one that is one of the 25 most conserved epitopes (for its epitope type, e.g., B cell, CD4 T cell, CD8 T cell) identified in a sequence alignment and analysis. In some embodiments, a conserved target epitope is one that is one of the 30 most conserved epitopes (for its epitope type, e.g. B cell, CD4 T cell, CD8 T cell) identified in a sequence alignment and analysis. In some embodiments, a conserved target epitope is one that is one of the 35 most conserved epitopes (for its epitope type, e.g. B cell, CD4 T cell, CD8 T cell) identified in a sequence alignment and analysis. In some embodiments, a conserved target epitope is one that is one of the 40 most conserved epitopes (for its epitope type, e.g., B cell, CD4 T cell, CD8 T cell) identified in a sequence alignment and analysis. In some embodiments, a conserved target epitope is one that is one of the 50 most conserved epitopes (for its epitope type, e.g., B cell, CD4 T cell, CD8 T cell) identified in a sequence alignment and analysis. Examples of sequence alignments and analyses. Are described herein. For example, steps or methods for selecting or identifying conserved large sequences may first include performing a sequence alignment and analysis of a particular number of coronavirus sequences to determine sequence similarity or identity amongst the group of analyzed sequences. In some embodiments, the sequences used for alignments may include human and animal sequences. In certain embodiments, the sequences used for alignments include one or more SARS-CoV-2 human strains or variants in current circulation; one or more coronaviruses that has caused a previous human outbreak; one or more coronaviruses isolated from animals selected from a group consisting of bats, pangolins, civet cats, minks, camels, and other animal receptive to coronaviruses; and/or one or more coronaviruses that cause the common cold. In some embodiments, the conserved large sequences are identified by: performing a sequence alignment and analysis of a particular number of coronavirus sequences to determine sequence similarity or identity amongst the group of analyzed sequences. The conserved large sequences are those that are among the most highly conserved sequences identified in the analysis. For example, the conserved large sequences may be the 2 most highly conserved sequences identified. In some embodiments, the conserved large sequences may be the 5 most highly conserved sequences identified. In some embodiments, the conserved large sequences may be the 8 most highly conserved sequences identified. In some embodiments, the conserved large sequences may be the 10 most highly conserved sequences identified. In some embodiments, the conserved large sequences may be the 15 most highly conserved sequences identified. In some embodiments, the conserved large sequences may be the 20 most highly conserved sequences identified. In some embodiments, the conserved large sequences may be the 30 most highly conserved sequences identified. In some embodiments, the conserved large sequences may be the 40 most highly conserved sequences identified. The present invention is not limited to the aforementioned thresholds. In some embodiments, the alignment and analysis for 50 or more sequences, 100 or more sequences, 200 or more sequences, 300 or more sequences, 400 or more sequences, 500 or more sequences, 1000 or more sequences, 2000 or more sequences, 3000 or more sequences, 4000 or more sequences, 5000 or more sequences, 10,000 or more sequences, 15,000 or more sequences, more than 15,000 sequences, etc., In some embodiments, the sequences used for alignments may include human and animal sequences. In certain embodiments, the sequences used for alignments include one or more SARS-CoV-2 human strains or variants in current circulation; one or more coronaviruses that has caused a previous human outbreak; one or more coronaviruses isolated from animals selected from a group consisting of bats, pangolins, civet cats, minks, camels, and other animal receptive to coronaviruses; and/or one or more coronaviruses that cause the common cold. In some embodiments, the one or more SARS-CoV-2 human strains or variants in current circulation are selected from: variant B.1.177; variant B.1.160, variant B.1.1.7 (UK), variant P.1 (Japan/Brazil), variant B.1.351 (South Africa), variant B.1.427 (California), variant B.1.429 (California), variant B.1.258; variant B.1.221; variant B.1.367; variant B.1.1.277; variant B.1.1.302; variant B.1.525; variant B.1.526, variant S:677H; variant S:677P: B.1.617.2-Delta, variant B.1.1.529-Omicron (BA.1); sub-variant Omicron (BA.1); sub-variant Omicron (BA.2); sub-variant Omicron (BA.3); sub-variant Omicron (BA.4); sub-variant Omicron (BA.5Y As discussed herein, the one or more conserved large sequences comprising target epitopes, are highly conserved among human and animal coronaviruses. For any of the embodiments herein, the epitopes that are selected may be those that achieve a particular score in a binding assay (for binding to an HLA molecule, for example.)

In certain embodiments, the one or more conserved coronavirus CD8+ T cell target epitopes are selected from: spike glycoprotein, Envelope protein, ORF1ab protein, ORF7a protein, ORF8a protein, ORF10 protein, or a combination thereof. In certain embodiments, the one or more conserved coronavirus CD8+ T cell target epitopes are selected from: S₂₋₁₀, S₁₂₂₀₋₁₂₂₈, S₁₀₀₀₋₁₀₀₈, S₉₅₈₋₉₆₆, E₂₀₋₂₈, ORF1ab₁₆₇₅₋₁₆₈₃, ORF1ab₂₃₆₆₋₂₃₇₁, ORF1ab₃₀₁₃₋₃₀₂₁, ORF1ab₃₁₈₃₋₃₁₉₁, ORF1ab₅₄₇₀₋₅₄₇₈, ORF1ab₆₇₄₉₋₆₇₅₇, ORF7b₂₈₋₃₄, ORF8a₇₃₋₈₁, ORF1₀₃₋₁₁, and ORF1₀₅₋₁₃. In certain embodiments, the one or more conserved coronavirus CD8+ T cell target epitopes are selected from SEQ ID NO: 2-29. In certain embodiments, the one or more conserved coronavirus CD8+ T cell target epitopes are selected from SEQ ID NO: 30-57.

In certain embodiments, the one or more conserved coronavirus CD4+ T cell target epitopes are selected from: spike glycoprotein, Envelope protein, Membrane protein, Nucleocapsid protein, ORF1a protein, ORF1ab protein, ORF6 protein, ORF7a protein, ORF7b protein, ORF8 protein, or a combination thereof. In certain embodiments, the one or more conserved coronavirus CD4+ T cell target epitopes are selected from: ORF1a₁₃₅₀₋₁₃₈₅, ORF1ab₅₀₁₉₋₅₀₃₃, ORF6₁₂₋₂₈s ORF1ab₆₀₈₈₋₆₁₀₂, ORF1ab₆₄₂₀₋₆₄₃₄, ORF1a₁₈₀₁₋₁₈₁₅, S₁₋₁₃, E₂₆₋₄₀, E₂₀₋₃₄, M₁₇₆₋₁₉₀, N₃₈₈₋₄₀₃, ORF7a₃₋₁₇, ORF7a₁₋₁₅, ORF7b₈₋₂₂, ORF7a₉₈₋₁₁₂, and ORF8₁₋₁₅. In certain embodiments, the one or more conserved coronavirus CD4+ T cell target epitopes are selected from SEQ ID NO: 58-73. In certain embodiments, the one or more conserved coronavirus CD4+ T cell target epitopes are selected from SEQ ID NO: 74-105.

In certain embodiments, the one or more conserved coronavirus B cell target epitopes are selected from Spike glycoprotein. In certain embodiments, the one or more conserved coronavirus B cell target epitopes are selected from: S₂₈₇₋₃₁₇, S₅₂₄₋₅₉₈, S₆₀₁₋₆₄₀, S₈₀₂₋₈₁₉, S₈₈₈₋₉₀₉, S₃₆₉₋₃₉₃, S₄₄₀₋₅₀₁, S₁₁₃₃₋₁₁₇₂, S₃₂₉₋₃₆₃, and S₁₃₋₃₇. In certain embodiments, the one or more coronavirus B cell target epitopes are selected from SEQ ID NO: 106-116 in certain embodiments, the one or more coronavirus B cell target epitopes are selected from SEQ ID NO: 117-138.

As previously discussed, in certain embodiments, the one or more conserved coronavirus B cell target epitopes are in the form of a large sequence, e.g., whole spike protein or partial spike protein (e.g., a portion of whole spike protein). In some embodiments, the whole spike protein or portion thereof is in its stabilized conformation. In certain embodiments, the transmembrane anchor of the spike protein (or portion thereof) has an intact S1-S2 cleavage site. In certain embodiments, the spike glycoprotein has two consecutive proline substitutions at amino acid positions 986 and 987, e.g., for stabilization. In certain embodiments, the spike protein or portion thereof has an amino acid substitution at amino acid position Tyr-83. In certain embodiments, the spike protein or portion thereof has an amino acid substitution at amino acid position Tyr-489. In certain embodiments, the spike protein or portion thereof has an amino acid substitution at amino acid position Gln-24. In certain embodiments, the spike protein or portion thereof has an amino acid substitution at amino acid position Asn-487. In certain embodiments, the spike protein or portion thereof has an amino acid substitution at one or more of: Tyr-83, Tyr-489, Gln-24, Gln-493, and Asn-487, e.g., the spike protein or portion thereof may comprise Tyr-489 and Asn-487, the spike protein or portion thereof may comprise Gln-493, the spike protein or portion thereof may comprise Tyr-505, etc. Tyr-489 and Asn-487 may help with interaction with Tyr 83 and Gln-24 on ACE-2. Gln-493 may help with interaction with Glu-35 and Lys-31 on ACE-2. Tyr-505 may help with interaction with Glu-37 and Arg-393 on ACE-2.

In certain embodiments, the composition comprises a mutation 682-RRAR-685→682-QQAQ-685 in the S1-32 cleavage site. In certain embodiments, the composition comprises at least one proline substitution. In certain embodiments, the composition comprises at least two proline substitutions, e.g., at position K986 and V987.

In certain embodiments, a large sequence derived from the spike glycoprotein is RBD. In certain embodiments, a large sequence derived from the spike glycoprotein is NTD. In certain embodiments, a large sequence derived from the spike glycoprotein is one or more large sequences, e.g., comprising both the RBD and NTD regions. In certain embodiments, a large sequence derived from the spike glycoprotein is recognized by neutralizing and blocking antibodies. In certain embodiments, a large sequence derived from the spike glycoprotein induces neutralizing and blocking antibodies. In certain embodiments, a large sequence derived from the spike glycoprotein induces neutralizing and blocking antibodies that recognize and neutralize the virus. In certain embodiments, a large sequence derived from the spike glycoprotein induces neutralizing and blocking antibodies that recognize the spike protein.

In certain embodiments, linkers are used, e.g., between epitopes, between large sequences, etc. In certain embodiments, the linker is from 2-10 amino acids in length. In certain embodiments, the linker is from 3-12 amino acids in length. In certain embodiments, the linker is from 5-15 amino acids in length. In certain embodiments, the linker is 10 or more amino acids in length. Non-limiting examples of linkers include AAY, KK, and GPGPG.

In some embodiments, the composition comprises the addition of a T4 fibritin-derived foldon trimerization domain. In some embodiments, the addition of a T4 fibritin-derived foldon trimerization domain increases immunogenicity by multivalent display. [0066] in certain embodiments, the composition further comprises a T cell attracting chemokine. For example, the composition may further comprise one or a combination of CCL5, CXCL9, CXCL10, CXCL11, or a combination thereof.

In certain embodiments, the composition further comprises a composition that promotes T cell proliferation. For example, the composition may further comprise IL-7, IL-15, IL-2, or a combination thereof.

In certain embodiments, the composition further comprises a molecular adjuvant. For example, the composition may further comprise one or a combination of CpG (e.g., CpG polymer) or flagellin.

In certain embodiments, the composition comprises a tag. For example, one or more of the large sequences may comprise a tag. In certain embodiments, the epitopes are in the form of two or more antigens, wherein one or more of the antigens comprise a tag. Non-limiting examples of tags include a His tag.

In certain embodiments, the “antigen delivery system” may refer to two delivery systems, e.g., a portion of the large sequences (or other components such as chemokines, etc.) may be encoded by one delivery system and a portion of the large sequences (or other components) may be encoded by a second delivery system (or a third delivery system, etc.).

Referring to the antigen delivery system, in certain embodiments the antigen delivery system is a vesicular stomatitis virus (VSV) vector. In certain embodiments, the antigen delivery system is an adenovirus (e.g., Ad26, Ad5, Ad35, etc.)

The large sequences are operatively linked to a promoter. In certain embodiments, the promoter is a generic promoter (e.g., CMV, CAG, etc.). In certain embodiments, the promoter is a lung-specific promoter (e.g., SpB, CD144). In certain embodiments, large sequences are operatively linked to the same promoter. In certain embodiments, one or more of the large sequences are operatively linked to a first promoter and one or more large sequences are operatively linked to a second promoter. In certain embodiments, the large sequences are operatively linked to two or more promoters, e.g., a portion are operatively linked to a first promoter, a portion are operatively linked to a second promoter, etc. In certain embodiments, the large sequences are operatively linked to three or more promoters, e.g., a portion is operatively linked to a first promoter, a portion is operatively linked to a second promoter, a portion is operatively linked to a third promoter, etc. In certain embodiments, the first promoter is the same as the second promoter. In certain embodiments the second promoter is different from the first promoter. In certain embodiments, the promoter is a generic promoter (e.g., CMV, CAG, etc.). In certain embodiments, the promoter is a lung-specific promoter (e.g., SpB, CD144) promoter.

In certain embodiments, the antigen delivery system or a separate antigen delivery system encodes a T cell attracting chemokine. In certain embodiments, the antigen delivery system or a separate antigen delivery system encodes a composition that promotes T cell proliferation. In certain embodiments, the antigen delivery system or a separate antigen delivery system encodes both a T cell attracting chemokine and a composition that promotes T cell proliferation. In certain embodiments, the antigen delivery system or a separate antigen delivery system encodes a molecular adjuvant. In certain embodiments, the antigen delivery system or a separate antigen delivery system encodes a T cell attracting chemokine, a composition that promotes T cell proliferation and a molecular adjuvant. In certain embodiments, the antigen delivery system or a separate antigen delivery system encodes a T cell attracting chemokine and a molecular adjuvant. In some embodiments, the antigen delivery system or a separate antigen delivery system encodes a composition that promotes T cell proliferation and a molecular adjuvant.

In certain embodiments, the T cell attracting chemokine is CCL5, CXCL9, CXCL10, CXCL11, or a combination thereof. In certain embodiments, the composition that promotes T cell proliferation is IL-7 or IL-15 or IL-2. In some embodiments, the molecular adjuvant is CpG (e.g., CpG polymer), flagellin, etc.).

In certain embodiments, the T cell attracting chemokine is operatively linked to a lung-specific promoter (e.g., SpB, CD144). In certain embodiments, the T cell attracting chemokine is operatively linked to a generic promoter (e.g., CMV, CAG, etc.). In certain embodiments, the composition that promotes T cell proliferation is operatively linked to a lung-specific promoter (e.g., SpB, CD144). In certain embodiments, the composition that promotes T cell proliferation is operatively linked to a generic promoter (e.g., CMV, CAG, etc.). In certain embodiments, the molecular adjuvant is operatively linked to a lung-specific promoter (e.g., SpB, CD144). In certain embodiments, the molecular adjuvant is operatively linked to a generic promoter (e.g., CMV, CAG, etc.). In certain embodiments, the T cell attracting chemokine and the composition that promotes T cell proliferation are driven by the same promoter. In certain embodiments, the T cell attracting chemokine and the composition that promotes T cell proliferation are driven by different promoters. In certain embodiments, the molecular adjuvant, the T cell attracting chemokine, and the composition that promotes T cell proliferation are driven by the same promoter. In certain embodiments, the molecular adjuvant, the T cell attracting chemokine, and the composition that promotes T cell proliferation are driven by different promoters. In certain embodiments, the molecular adjuvant and the composition that promotes T cell proliferation are driven by different promoters. In certain embodiments, the molecular adjuvant and the T cell attracting chemokine are driven by different promoters.

In certain embodiments, the T cell attracting chemokine and the composition promoting T cell proliferation are separated by a linker, in certain embodiments, the linker comprises T2A. In certain embodiments, the linker comprises E2A. In certain embodiments, the linker comprises P2A. In certain embodiments, the linker is selected from T2A, E2A, and P2A.

Referring to the antigen delivery system, in certain embodiments, a linker is disposed between each open reading frame. In certain embodiments, a different linker is disposed between each open reading frame. In certain embodiments, the same linker may be used between particular open reading frames and a different linker may be used between other open reading frames.

In some embodiments, the vaccine composition is administered using an adenovirus.

The composition herein may be used to prevent a coronavirus disease in a subject. The composition herein may be used to prevent a coronavirus infection prophylactically in a subject. The composition herein may be used to elicit an immune response in a subject. The term “subject” herein may refer to a human, a non-human primate, an animal such as a mouse, rat, cat, dog, other animal that is susceptible to coronavirus infection, or other animal used for preclinical modeling. The composition herein may prolong an immune response induced by the pan-coronavirus recombinant vaccine composition and increases T-cell migration to the lungs. In certain embodiments, the composition induces resident memory T cells (Trm). In some embodiments, the vaccine composition induces efficient and powerful protection against the coronavirus disease or infection. In some embodiments, the vaccine composition induces production of antibodies (Abs), CD4+ T helper (Th1) cells, and CD8+ cytotoxic T-cells (CTL). In some embodiments, the composition that promotes T cell proliferation helps to promote long term immunity. In some embodiments, the T-cell attracting chemokine helps pull T-cells from circulation into the lungs.

In certain embodiments, the composition further comprises a pharmaceutical carrier.

The present invention includes any of the vaccine compositions described herein, e.g., the aforementioned vaccine compositions for delivery with nanoparticles, e.g., lipid nanoparticles. For example, the present invention includes the vaccine compositions herein encapsulated in a lipid nanoparticle.

The present invention includes the compositions described herein comprising and/or encoding a trimerized SARS-CoV-2 receptor-binding domain (RBD) and one or more highly conserved SARS-CoV-2 sequences selected from structural proteins (e.g., nucleoprotein, etc.) and non-structural protein (e.g., Nsp4, etc.). In some embodiments, the trimerized SARS-CoV-2 receptor-binding domain (RBD) sequence is modified by the addition of a T4 fibritin-derived foldon trimerization domain. In some embodiments, the addition of a T4 fibritin-derived foldon trimerization domain increases immunogenicity by multivalent display.

The present invention also features methods of producing a pan-coronavirus recombinant vaccine compositions of the present invention.

For example, in some embodiments, the method comprises selecting at least conserved large sequences comprising: one or more coronavirus B-cell epitopes; one or more coronavirus CD4+ T cell epitopes; one or more coronavirus CD8+ T cell epitopes. In other embodiments, the method comprises selecting at least two conserved large sequences comprising: one or more coronavirus B-cell epitopes; one or more coronavirus CD4-+ T cell epitopes; one or more coronavirus CD8+ T cell epitopes. At least one large sequence is derived from a non-spike protein. The method further comprises synthesizing an antigen or antigens comprising the selected large sequences. In some embodiments, the method comprises selecting: one or more conserved large sequences comprising one or more coronavirus B-cell epitopes; one or more coronavirus CD4+ T cell epitopes; and one or more coronavirus CD8+ T cell epitopes. At least one large sequence is derived from a non-spike protein. The method further comprises synthesizing an antigen or antigens comprising the selected large sequences. In some embodiments, the method further comprises introducing the vaccine composition to a pharmaceutical carrier. The steps for selecting the one or more conserved large sequences are disclosed herein. Methods for synthesizing recombinant proteins are well known to one of ordinary skill in the art. The vaccine compositions are disclosed herein. In some embodiments, the vaccine composition is in the form of DNA, RNA, modified RNA, protein (or peptide), or a combination thereof.

In some embodiments, the method comprises selecting: at least one conserved large sequence comprising: one or more coronavirus B-cell epitopes; one or more coronavirus CD4+ T cell epitopes; and one or more coronavirus CD8+ T cell epitopes. At least one large sequence is derived from a non-spike protein. The method further comprises synthesizing an antigen delivery system encoding the selected large sequences. In some embodiments, the method further comprises introducing the vaccine composition to a pharmaceutical carrier. The steps for selecting the one or more conserved large sequences are disclosed herein. Methods for synthesizing antigen delivery systems are well known to one of ordinary skill in the art. The vaccine compositions are disclosed herein. In some embodiments, the vaccine composition is in the form of DNA, RNA, modified RNA, protein (or peptide), or a combination thereof.

As an example, steps or methods for selecting or identifying conserved large sequences may first include performing a sequence alignment and analysis of a particular number of coronavirus sequences, e.g., 50 or more sequences, 100 or more sequences, 200 or more sequences, 300 or more sequences, 400 or more sequences, 500 or more sequences, 1000 or more sequences, 2000 or more sequences, 3000 or more sequences, 4000 or more sequences, 5000 or more sequences, 10,000 or more sequences, 15,000 or more sequences, more than 15,000 sequences, etc., to determine sequence similarity or identity amongst the group of analyzed sequences. In some embodiments, the sequences used for alignments may include human and animal sequences. In certain embodiments, the sequences used for alignments include one or more SARS-CoV-2 human strains or variants in current circulation; one or more coronaviruses that has caused a previous human outbreak; one or more coronaviruses isolated from animals selected from a group consisting of bats, pangolins, civet cats, minks, camels, and other animal receptive to coronaviruses; and/or one or more coronaviruses that cause the common cold. In some embodiments, the one or more SARS-CoV-2 human strains or variants in current circulation are selected from: variant B.1.177; variant B.1.160, variant B.1.1.7 (UK), variant P.1 (Japan/Brazil), variant 8.1.351 (South Africa), variant B.1.427 (California), variant B.1.429 (California), variant B.1.258; variant B.1.221; variant B.1.367; variant B.1.1.277; variant B.1.1.302; variant B.1.525; variant B.1.526, variant S:677H; variant S:677P; B.1.617.2-Delta, variant B.1.1.529-Omicron (BA.1); sub-variant Omicron (BA.1); sub-variant Omicron (BA.2); sub-variant Omicron (BA.3); sub-variant Omicron (BA.4); sub-variant Omicron (BA.5). In some embodiments, the one or more coronaviruses that cause the common cold are selected from: 229E alpha coronavirus, NL63 alpha coronavirus, OC43 beta coronavirus, and HKU1 beta coronavirus. In some embodiments, the conserved large sequences may be considered the 2 most highly conserved sequences of the identified large sequences in the alignment. In some embodiments, the conserved large sequences may be considered the 5 most highly conserved sequences of the identified large sequences in the alignment. In some embodiments, the conserved large sequences may be considered the 10 most highly conserved sequences of the identified large sequences in the alignment. In some embodiments, the conserved large sequences may be considered the 15 most highly conserved sequences of the identified large sequences in the alignment.

The present invention also features methods for preventing coronavirus disease. The method comprises administering to a subject a therapeutically effective amount of a pan-coronavirus recombinant vaccine composition according to the present invention, wherein the composition elicits an immune response in the subject and helps prevent coronavirus disease.

The present invention also features methods for preventing a coronavirus infection prophylactically in a subject. In some embodiments, the method comprises administering to the subject a prophylactically effective amount of a pan-coronavirus recombinant vaccine composition according to the present invention, wherein the vaccine composition prevents coronavirus infection.

The present invention also features methods for eliciting an immune response in a subject, comprising administering to the subject a composition according to the present invention, wherein the vaccine composition elicits an immune response in the subject. The present invention also features methods comprising: administering to a subject a pan-coronavirus recombinant vaccine composition according to the present invention, wherein the composition prevents virus replication in the lungs, the brain, and other compartments where the virus replicates. The present invention also features methods comprising: administering to the subject a pan-coronavirus recombinant vaccine composition according to the present invention, wherein the composition prevents cytokine storm in the lungs, the brain, and other compartments where the virus replicates. The present invention also features methods comprising: administering to the subject a pan-coronavirus recombinant vaccine composition according to the present invention, wherein the composition prevents inflammation or inflammatory response in the lungs, the brain, and other compartments where the virus replicates. The present invention also features methods comprising: administering to the subject a pan-coronavirus recombinant vaccine composition according to the present invention, wherein the composition improves homing and retention of T cells in the lungs, the brain, and other compartments where the virus replicates. The present invention also features methods for preventing coronavirus disease in a subject; the method comprising: administering to the subject a pan-coronavirus recombinant vaccine composition according to the present invention, wherein the composition induces memory B and T cells. The present invention also features methods for prolonging an immune response induced by a pan-coronavirus recombinant vaccine and increasing T-cell migration to the lungs, the method comprising: co-expressing a T-cell attracting chemokine, a composition that promotes T cell proliferation, and a pan-coronavirus recombinant vaccine according to the present invention. The present invention also features methods for prolonging the retention of memory T-cell into the lungs induced by a pan coronavirus vaccine and increasing virus-specific tissue resident memory T-cells (TRM cells), the method comprising: co-expressing a T-cell attracting chemokine, a composition that promotes T cell proliferation, and a pan-coronavirus recombinant vaccine according to the present invention. The present invention also features methods comprising: administering to the subject a pan-coronavirus recombinant vaccine composition according to the present invention, wherein the composition prevents the development of mutation and variants of a coronavirus.

For the sake of brevity, it is noted that the vaccine compositions referred to in the aforementioned methods include the vaccine compositions previously discussed, the embodiments described below, and the embodiments in the figures.

In some embodiments, the vaccine composition is administered through an intravenous route (i.v.), an intranasal route (i.n.), or a sublingual route (s.l.) route.

In some embodiments, the vaccine composition is administered using an adenovirus or other appropriate delivery system.

As previously discussed, the composition herein may be used to prevent a coronavirus disease in a subject. The composition herein may be used to prevent a coronavirus infection prophylactically in a subject. The composition herein may be used to elicit an immune response in a subject. The term “subject” herein may refer to a human, a non-human primate, an animal such as a mouse, rat, cat, dog, other animal that is susceptible to coronavirus infection, or other animal used for preclinical modeling. The composition herein may prolong an immune response induced by the pan-coronavirus recombinant vaccine composition and increases T-cell migration to the lungs. In certain embodiments, the composition induces resident memory T cells (Trm). In some embodiments, the vaccine composition induces efficient and powerful protection against the coronavirus disease or infection. In some embodiments, the vaccine composition induces production of antibodies (Abs), CD4+ T helper (Th1) cells, and CD8+ cytotoxic T-cells (CTL). In some embodiments, the composition that promotes T cell proliferation helps to promote long term immunity. In some embodiments, the T-cell attracting chemokine helps pull T-cells from circulation into the lungs.

The present invention also features oligonucleotide compositions. For example, the present invention includes oligonucleotides disclosed in the sequence listings. The present invention also includes oligonucleotides in the form of antigen delivery systems. The present invention also includes oligonucleotides encoding the conserved large sequences disclosed herein. The present invention also includes oligonucleotide compositions comprising one or more oligonucleotides encoding any of the vaccine compositions according to the present invention. In some embodiments, the oligonucleotide comprises DNA. In some embodiments, the oligonucleotide comprises modified DNA. In some embodiments, the oligonucleotide comprises RNA. In some embodiments, the oligonucleotide comprises modified RNA. In some embodiments, the oligonucleotide comprises mRNA. In some embodiments, the oligonucleotide comprises modified mRNA.

The present invention also features peptide compositions. For example, the present invention includes peptides disclosed in the sequence listings. The present invention also includes peptide compositions comprising any of the vaccine compositions according to the present invention. The present invention also includes peptide compositions comprising any of the conserved large sequences according to the present invention.

For the sake of brevity, it is noted that the vaccine compositions referred to in the aforementioned oligonucleotide and peptide compositions include the vaccine compositions previously discussed, the embodiments described below, and the embodiments in the figures.

The present invention also features a pan-coronavirus recombinant vaccine composition comprising SEQ ID NO 139-147 (Table 9). The present invention also features a pan-coronavirus recombinant vaccine composition at least 99% identical to SEQ ID NO: 139-147 (Table 9).

The present invention also features a method comprising: administering a first pan-coronavirus recombinant vaccine dose using a first delivery system, and administering a second vaccine dose using a second delivery system, wherein the first and second delivery system are different. In some embodiments, the first delivery system may comprise a RNA, a modified mRNA, or a peptide delivery system. In some embodiments, the second delivery system may comprise a RNA, a modified mRNA, or a peptide delivery system. In some embodiments, the peptide delivery system is an adenovirus. In some embodiments, the adenovirus delivery system is Ad26, Ad5, Ad35, or a combination thereof. In some embodiments, the peptide delivery system is a vesicular stomatitis virus (VSV) vector. In some embodiments, the second vaccine dose is administered 14 days after the first vaccine dose.

The present invention also features a method comprising: administering a pan-coronavirus recombinant vaccine composition according to the present invention: and administering at least one T-cell attracting chemokine after administering the pan-coronavirus recombinant vaccine composition. In some embodiments, the vaccine composition is administered via a RNA, a modified mRNA, or a peptide delivery system. In some embodiments, the T-cell attracting chemokine is administered via a RNA, a modified mRNA, or a peptide delivery system. In some embodiments, the peptide delivery system is an adenovirus.

In some embodiments, the adenovirus delivery system is Ad26, Ad5, Ad35, or a combination thereof. In some embodiments, the peptide delivery system is a vesicular stomatitis virus (VSV) vector. In some embodiments, the T-cell attracting chemokine is administered 8 days after administering days after the vaccine composition. In some embodiments, the T-cell attracting chemokine is administered 14 days after administering days after the vaccine composition. In some embodiments, the T-cell attracting chemokine is administered 30 days after administering days after the vaccine composition. In some embodiments, the T-cell attracting chemokine is CCL5, CXCL9, CXCL10, CXCL11, or a combination thereof.

The present invention also features a method comprising: administering a pan-coronavirus recombinant vaccine composition according to the present invention; administering at least one T-cell attracting chemokine after administering the pan-coronavirus recombinant vaccine composition; and administering at least one cytokine after administering the T-cell attracting chemokine. In some embodiments, the vaccine composition is administered via a RNA, a modified mRNA, or a peptide delivery system. In some embodiments, the T-cell attracting chemokine is administered via a RNA, a modified mRNA, or a peptide delivery system. In some embodiments, the cytokine is administered via a RNA, a modified mRNA, or a peptide delivery system. In some embodiments, the peptide delivery system is an adenovirus. In some embodiments, the adenovirus delivery system is Ad26, Ad5, Ad35, or a combination thereof. In some embodiments, the peptide delivery system is a vesicular stomatitis virus (VSV) vector. In some embodiments, the T-cell attracting chemokine is administered 14 days after administering the vaccine composition. In some embodiments, the T-cell attracting chemokine is CCL5, CXCL9, CXCL10, CXCL11, or a combination thereof. In some embodiments, the cytokine is administered 10 days after administering the T-cell attracting chemokine. In some embodiments, the cytokine is IL-7, IL-15, IL2 or a combination thereof.

The present invention also features a method comprising: administering a pan-coronavirus recombinant vaccine composition according to the present invention; administering one or more T-cell attracting chemokine after administering the pan-coronavirus recombinant vaccine composition; and administering one or more mucosal chemokine(s). In some embodiments, the vaccine composition is administered using an adenovirus. In some embodiments, the T-cell attracting chemokine is administered via a RNA, a modified mRNA, or a peptide delivery system, or other delivery system. In some embodiments, the mucosal chemokine is administered via a RNA, a modified mRNA, or a peptide delivery system, or other delivery system. In some embodiments, the adenovirus is Ad26, Ad5, Ad35, or a combination thereof. In some embodiments, the T-cell attracting chemokine is administered 14 days after administering the vaccine composition. In some embodiments, the T-cell attracting chemokine is CCL5, CXCL9, CXCL10, CXCL11, or a combination thereof. In some embodiments, the mucosal chemokine is administered 10 days after administering the T-cell attracting chemokine. In some embodiments, the mucosal chemokine is CCL25, CCL28, CXCL14, or CXCL17, or a combination thereof.

For the sake of brevity, it is noted that the vaccine compositions referred to in the aforementioned methods include the vaccine compositions previously discussed, the embodiments described below, and the embodiments in the figures.

As previously discussed, in some embodiments, the vaccine compositions are for use in humans. In some embodiments, the vaccine compositions are for use in animals, e.g., cats, dogs, etc. In some embodiments, the vaccine composition comprises human CXCL-11 and/or human IL-7 (or IL-15, IL-2). In some embodiments, the vaccine composition comprises animal CLCL-11 and/or animal IL-7 (or IL-15, IL-2).

The present invention includes vaccine compositions in the form of a rVSV-panCoV vaccine composition. The present invention includes vaccine compositions in the form of a rAdV-panCoV vaccine composition.

The present invention also includes nucleic acids for use in the vaccine compositions herein. The present invention also includes vectors for use in the vaccine compositions herein. The present invention also includes fusion proteins for use in the vaccine compositions herein. The present invention also includes immunogenic compositions for use in the vaccine compositions herein.

The vaccine compositions herein may be designed to elicit both high levels of virus-blocking and virus-neutralizing antibodies as well as CD4+ T cells and CD8+ T cells in adults 18 to 55 years. The vaccine compositions herein may be designed to elicit both high levels of virus-blocking and virus-neutralizing antibodies as well as CD4+ T cells and CD8+ T cells in adults 55 to 65 years of age. The vaccine compositions herein may be designed to elicit both high levels of virus-blocking and virus-neutralizing antibodies as well as CD4+ T cells and CD8+ T cells in adults 65 to 85 years of age. The vaccine compositions herein may be designed to elicit both high levels of virus-blocking and virus-neutralizing antibodies as well as CD4+ T cells and CD8+ T cells in adults 85 to 100 years of age. The vaccine compositions herein may be designed to elicit both high levels of virus-blocking and virus-neutralizing antibodies as well as CD4+ T cells and CD8+ T cells in children 12 to 18 years of age. The vaccine compositions herein may be designed to elicit both high levels of virus-blocking and virus-neutralizing antibodies as well as CD4+ T cells and CD8+ T cells in children under 12 years of age.

The present invention is not limited to vaccine compositions. For example, in certain embodiments, one or more of the conserved large sequences are used for detecting coronavirus and/or diagnosing coronavirus infection.

As previously discussed, in some embodiments, the one or more conserved large sequences are highly conserved among human and animal coronaviruses. In some embodiments, the conserved large sequence is one that is among the most highly conserved large sequences identified in a sequence alignment and analysis of a particular number of coronavirus sequences. For example, the conserved large sequence may be the 2 most highly conserved large sequences identified. In some embodiments, the conserved large sequences may be the 5 most highly conserved large sequences identified. In some embodiments, the conserved large sequences may be the 8 most highly conserved large sequences identified. In some embodiments, the conserved large sequences may be the 10 most highly conserved large sequences identified. In some embodiments, the conserved large sequences may be the 15 most highly conserved large sequences identified. In some embodiments, the conserved large sequences may be the 20 most highly conserved large sequences identified. In some embodiments, the conserved large sequences may be the 30 most highly conserved large sequences identified. In some embodiments, the conserved large sequences may be the 40 most highly conserved large sequences identified. In some embodiments, the one or more conserved. In some embodiments, the conserved large sequences may be the 5 most highly conserved large sequences identified are derived from at least one of SARS-CoV-2 protein. In some embodiments, the one or more conserved In some embodiments, the conserved large sequences may be the 5 most highly conserved large sequences identified are derived from one or more of: one or more SARS-CoV-2 human strains or variants in current circulation; one or more coronaviruses that has caused a previous human outbreak; one or more coronaviruses isolated from animals selected from a group consisting of bats, pangolins, civet cats, minks, camels, and other animal receptive to coronaviruses; or one or more coronaviruses that cause the common cold. In some embodiments, the one or more SARS-CoV-2 human strains or variants in current circulation are selected from variant B.1.177; variant B.1.160, variant B.1.1.7 (UK), variant P.1 (Japan/Brazil), variant B.1.351 (South Africa), variant B.1.427 (California), variant B.1.429 (California), variant B.1.258; variant B.1.221; variant 8.1.367; variant 8.1.1.277; variant B.1.1.302; variant B.1.525; variant B1.526, variant S:677H; variant S:677P; B.1.617.2-Delta, variant B.1.1.529-Omicron (BA.1); sub-variant Omicron (BA.1); sub-variant Omicron (BA.2); sub-variant Omicron (BA.3); sub-variant Omicron (BA.4); sub-variant Omicron (BA.5). In some embodiments, the one or more coronaviruses that cause the common cold are selected from: 229E alpha coronavirus, NL63 alpha coronavirus, OC43 beta coronavirus, and HKU1 beta coronavirus. In some embodiments, the vaccine composition is for humans. In some embodiments, the vaccine composition is for animals.

The present invention also features a method of producing a pan-coronavirus composition, the method comprising selecting at least one large sequence(s) according to the present invention and synthesizing one or more antigens comprising the selected large sequence(s). The present invention also features a method of producing a pan-coronavirus composition, the method comprising selecting at least one conserved large sequence(s); and synthesizing an antigen delivery system that encodes the selected large sequence(s).

The present invention also includes a pan-coronavirus recombinant vaccine composition, the composition comprising one or more large sequences, each of the one or more large sequences comprises at least one of: whole spike protein or a portion thereof; one or more conserved coronavirus CD4+ T cell target epitope; and one or more conserved coronavirus CD8+ T cell target epitope; wherein at least one epitope is derived from a non-spike protein.

In some embodiments, the one or more conserved epitopes are highly conserved among human and animal coronaviruses. In some embodiments, the one or more conserved epitopes are derived from at least one of SARS-CoV-2 protein. In some embodiments, the composition comprises 2-20 CD8+ T cell target epitopes. In some embodiments, the composition comprises 2-20 CD4+ T cell target epitopes. In some embodiments, the one or more conserved coronavirus CD4+ T cell target epitopes selected from SEQ ID NO: 58-105 (ORF1a1350-1365, ORF1ab5019-5033, ORF612-26, ORF1ab6088-6102, ORF1ab6420-6434, ORF1a1801-1815, S1-13, E26-40, E20-34, M176-190, N388-403, ORF7a3-17, ORF7a1-15, ORF7b8-22, ORF7a98-112, and ORF81-15.). In some embodiments, the one or more conserved coronavirus CD8+ T cell target epitopes selected from SEQ ID NO: 106-138 (S287-317, S524-598, S601-640, S802-819, S888-909, S369-393, S440-501, S1133-1172, S329-363, and S13-37).

The present invention also features a pan-coronavirus recombinant vaccine composition, the composition comprising one or more large sequences, each of the one or more large sequences comprises at least one of: one or more conserved coronavirus B-cell target epitope; one or more conserved coronavirus CD4+ T cell target epitope; and/or one or more conserved coronavirus CD8+ T cell target epitope, wherein at least one epitope is derived from a non-spike protein.

In some embodiments, the one or more conserved epitopes are derived from at least one of SARS-CoV-2 proteins. In some embodiments, the composition comprises 2-20 CD8+ T cell target epitopes.

In some embodiments, the composition comprises 2-20 CD4+ T cell target epitopes. In some embodiments, the one or more conserved coronavirus CD4+ T cell target epitopes selected from SEQ ID NO 58-105 (ORF1a1350-1365, ORF1ab5019-5033, ORF612-26, ORF1ab6088-6102, ORF1ab6420-6434, ORF1a1801-1815, S1-13, E26-40, E20-34, M176-190, N388-403, ORF7a3-17, ORF7a1-15, ORF7b8-22, ORF7a98-112, and ORF81-15.).

In some embodiments, the one or more conserved coronavirus CD8+ T cell target epitopes selected from SEQ ID NO: 106-138 (S287-317, 3524-598, S601-640, S802-819, 3888-909, S369-393, S440-501, S1133-1172, S329-363, and S13-37).

In some embodiments, the one or more conserved coronavirus B-cell target epitopes selected from SEQ ID NO: 2-57 (S2-10, S1220-1228, S1000-1008, S958-966, E20-28, ORF1ab1675-1683, ORF1ab2363-2371, ORF1ab3013-3021, ORF1ab3183-3191, ORF1ab5470-5478, ORF1ab6749-6757, ORF7b26-34, ORF8a73-81, ORF103-11, and ORF105-13);

The present invention also features a pan-coronavirus recombinant vaccine composition, the composition comprising an antigen delivery system encoding one or more large sequences, the large sequences comprise at least one of: one or more conserved coronavirus B-cell target epitopes; one or more conserved coronavirus CD4+ T cell target epitopes; and/or one or more conserved coronavirus CD8+ T cell target epitopes; wherein at least one epitope is derived from a non-spike protein.

In some embodiments, the antigen delivery system is an adenovirus-based antigen delivery system. In some embodiments, the adenovirus-based antigen delivery system is Ad26, Ad5, Ad35, or a combination thereof. In some embodiments, the antigen delivery system further encodes a T cell attracting chemokine. In some embodiments, the antigen delivery system further encodes a composition that promotes T cell proliferation. In some embodiments, the antigen delivery system further encodes a molecular adjuvant. In some embodiments, the large sequences are operatively linked to a lung-specific promoter.

In some embodiments, the one or more conserved coronavirus B-cell target epitopes selected from SEQ ID NO: 2-57 (S2-10, S1220-1228, S1000-1008, S958-966, E20-28, ORF1ab1675-1683, ORF1ab2363-2371, ORF1ab3013-3021, ORF1ab3183-3191, ORF1ab5470-5478, ORF1ab6749-6757, ORF7b26-34, ORF8a73-81, ORF103-11, and ORF105-13). In some embodiments, the one or more conserved coronavirus CD4+ T cell target epitopes selected from SEQ ID NO: 58-105 (ORF1a1350-1365, ORF1ab5019-5033, ORF612-26, ORF1ab6088-6102. ORF1ab6420-6434, ORF1a1801-1815, S1-13, E26-40, E20-34, M176-190, N388-403, ORF7a3-17, ORF7a1-15, ORF7b8-22, ORF7a98-112, and ORF81-15.). In some embodiments, the one or more conserved coronavirus CD8+ T cell target epitopes selected from SEQ ID NO: 106-138 (S287-317, S524-598, S601-640, S802-819, 3888-909, S369-393, S440-501, S1133-1172, S329-363, and S13-37).

In some embodiments, the partial spike protein comprises a trimerized SARS-CoV-2 receptor-binding domain (RBD). In some embodiments, the whole spike protein or partial spike protein has an intact S1-S2 cleavage site. In some embodiments, the spike protein is stabilized with proline substitutions at amino acid positions 986 and 987.

The present invention also features a pan-coronavirus recombinant vaccine composition comprising one of SEQ ID NO: 139-147.

The present invention also includes the corresponding nucleic acid sequences for any of the protein sequences herein. The present invention also includes the corresponding protein sequences for any of the nucleic acid sequences herein.

Embodiments herein may comprise whole spike protein or a portion of spike protein. Whole spike protein and a portion thereof is not limited to a wild type or original sequence and may include spike protein or a portion thereof with one or more modifications and/or mutations, such as point mutations, deletions, etc.; including the mutations described herein such as those for improving stability.

Embodiments of the present invention can be freely combined with each other if they are not mutually exclusive.

Any feature or combination of features described herein are included within the scope of the present invention provided that the features included in any such combination are not mutually inconsistent as will be apparent from the context, this specification, and the knowledge of one of ordinary skill in the art.

Additional advantages and aspects of the present invention are apparent in the following detailed description and claims.

### DESCRIPTION OF THE SEVERAL VIEWS OF THE DRAWING(S)

The features and advantages of the present invention will become apparent from a consideration of the following detailed description presented in connection with the accompanying drawings in which:

FIG. 1 shows a schematic view of an example of a large sequence pan-coronavirus recombinant vaccine composition. Each large sequence in the recombinant vaccine composition may comprise epitopes. CD8+ T cell epitopes are shown with a square, CD4+ T cell epitopes are shown with a circle and B-cell epitopes are shown with a diamond. Each shape (square, circle, or diamond) may represent a variety of different epitopes and is not limited to a singular epitope. The multi-epitope pan-coronavirus vaccines are not limited to a specific combination of large sequences as shown. The large sequence pan-coronavirus vaccines may comprise a various number of large sequences.

FIG. 2A shows an evolutionary comparison of genome sequences among beta-Coronavirus strains isolated from humans and animals. A phylogenetic analysis performed between SARS-CoV-2 strain sp (obtained from humans (Homo Sapiens (black)), along with the animal's SARS-like Coronaviruses genome sequence (SL-CoVs) sequences obtained from bats (Rhinolophus affinis, Rhinolophus malayanus (red)), pangolins (Manis javanica (blue)), civet cats (Paguma larvata (green)), and camels (Camelus dromedaries (Brown)). The included SARS-CoV/MERS-CoV strains are from previous outbreaks (obtained from humans (Urbani, MERS-CoV, OC43, NL63, 229E, HKU1-genotype-B), bats (WIV16, WIV1, YNLF-31C, Rs672, recombinant strains), camel (Camelus dromedaries, (KT368891.1, MN514967.1, KF917527.1, NC_028752.1), and civet (Civet007, A022, B039)). The human SARS-CoV-2 genome sequences are represented from six continents.

FIG. 28 shows an evolutionary analysis performed among the human-SARS-CoV-2 genome sequences reported from six continents and SARS-CoV-2 genome sequences obtained from bats (Rhinolophus affinis, Rhinolophus malayanus), and pangolins (Manis javanica)).

FIG. 3A shows lungs, heart, kidneys, intestines, brain, and testicles express ACE2 receptors and are targeted by SARS-CoV-2 virus. SARS-CoV-2 virus docks on the Angiotensin converting enzyme 2 (ACE2) receptor via spike surface protein.

FIG. 3B shows a System Biology Analysis approach utilized in the present invention.

FIG. 4 shows sequence homology analysis for SARS-CoV-2, common cold CoV strains, MERS, SARS-CoV-Urbani and animal CoVs with SARS-CoV-2 Wuhan Strain (Query strain; hCoV-19batYN01). Five fragments SARS-CoV-2 genome were found to be highly conserved (1 bp-1580 bp (fragment 1), 3547 bp-12830 bp (fragment 2), 17472 bp-21156 bp (fragment 3), 22584 bp-24682 bp (fragment 4), and 26193 bp-27421 bp (fragment 5).

FIG. 5 shows sequence homology analysis for fragment 1 (1 bp-1580 bp) which comprises portions of ORF1a/b. The Query sequence (1-1580 bp hCoV-19/batYN1) was BLAST against all the SARS-CoV-2 VOCs, human CoV strains, CoV strains from bats, pangolin, civet cat. 28 variants/strains were found with significant homology for this queried region.

FIG. 6 shows sequence homology analysis for fragment 2 (3547 bp-12830 bp). The Query sequence (3547-12830 bp hCoV-19/batYN01) was BLAST against all the SARS-CoV-2 VOCs, human CoV strains; CoV strains from bats, pangolin, civet cats. 30 variants/strains were found with significant homology for this queried region.

FIG. 7 shows sequence homology analysis for fragment 3 (17472 bp-21156 bp). The Query sequence (17472-21156 bp hCoV-19/batYN01) was BLAST against all the SARS-CoV-2 VOCs, human CoV strains, CoV strains from bats, pangolin, civet cats. 29 variants/strains were found with significant homology for this queried region.

FIG. 8 shows sequence homology analysis for fragment 4 (22584 bp-24682 bp) which comprises the spike protein. The Query sequence (22584-24682 bp hCoV-19/batYN01) was BLAST against all the SARS-CoV-2 VOCs, human CoV strains, CoV strains from bats, pangolin, civet cats. 29 variants/strains were found with significant homology for this queried region.

FIG. 9 shows sequence homology analysis for fragment 5 (26193 bp-27421 bp). The Query sequence (26193-27421 bp hCoV-19/batYN01) was BLAST against all the SARS-CoV-2 VOCs, human CoV strains, CoV strains from bats, pangolin, civet cats. 31 variants/strains were found with significant homology for this queried region.

FIG. 10 shows a sequence homology analysis to screen conservancy of potential SARS-CoV-2-derived human CD8+ T cell epitopes. Shown are the comparison of sequence homology for the potential CD8+ T cell epitopes among 81,963 SARS-CoV-2 strains (that currently circulate in 190 countries on 6 continents), the 4 major “common cold” Coronaviruses that cased previous outbreaks (i.e. hCoV-OC43, hCoV-229E, hCoV-HKU1-Genotype B, and hCoV-NL63), and the SL-CoVs that were isolated from bats, civet cats, pangolins and camels. Epitope sequences highlighted in yellow present a high degree of homology among the currently circulating 81,963 SARS-CoV-2 strains and at least a 50% conservancy among two or more humans SARS-CoV strains from previous outbreaks, and the SL-CoV strains isolated from bats, civet cats, pangolins and camels, as described herein. Homo Sapiens—black, bats (Rhinolophus affinis, Rhinolophus malayanus—red), pangolins (Manis javanica—blue), civet cats (Paguma larvata—green), and camels (Camelus dromedaries-brown).

FIG. 11A shows docking of highly conserved SARS-CoV-2-derived human CD8+ T cell epitopes to HLA-A*02:01 molecules, e.g., docking of the 27 high-affinity CD8+ T cell binder peptides to the groove of HLA-A*02:01 molecules.

FIG. 11B shows a summary of the interaction similarity scores of the 27 high-affinity CDB8+ T cell epitope peptides to HLA-A*02:01 molecules determined by protein-peptide molecular docking analysis. Black columns depict CD8+ T cell epitope peptides with high interaction similarity scores.

FIG. 12A shows an experimental design show CD8+ T cells are specific to highly conserved SARS-CoV-2 epitopes detected in COVID-19 patients and unexposed healthy individuals: PBMCs from HLA-A*02:01 positive COVID-19 patients (n=30) and controls unexposed healthy individuals (n=10) were isolated and stimulated overnight with 10 μM of each of the 27 SARS-CoV-2-derived CD8+ T cell epitopes. The number of IFN-γ-producing cells were quantified using ELISpot assay.

FIG. 12B shows the results from FIG. 12A. Dotted lines represent threshold to evaluate the relative magnitude of the response: a mean SFCs between 25 and 50 correspond to a medium/intermediate response whereas a strong response is defined for a mean SFCs >50.

FIG. 12C shows the results from experiments where PBMCs from HLA-A*02:01 positive COVID-19 patients were further stimulated for an additional 5 hours in the presence of mAbs specific to CD107a and CD107b, and Golgi-plug and Golgi-stop. Tetramers specific to Spike epitopes, CD107a/b and CD69 and TNF-expression were then measured by FACS. Representative FACS plot showing the frequencies of Tetramer+CD8+ T cells, CD107a % b+CD8+ T cells, CD69+CD8+ T cells and TNF-+CDB8+ T cells following priming with a group of 4 Spike CD8+ T cell epitope peptides. Average frequencies of tetramer+CD8+ T cells, CD107a/b+CD8+ T cells, CD69+CD8+ T cells and TNF-+CD8+ T cells.

FIG. 13A shows a timeline of immunization and immunological analyses for experiments testing the immunogenicity of genome-wide identified human SARS-CoV-2 CD8+ T epitopes in HLA-A*02:01/HLA-DRB1 double transgenic mice. Eight groups of age-matched HLA-A*02:01 transgenic mice (n=3) were immunized subcutaneously, on days 0 and 14, with a mixture of four SARS-CoV-2-derived human CD8+ T cell peptide epitopes mixed with PADRE CD4+ T helper epitope, delivered in alum and CpG1826 adjuvants. As a negative control, mice received adjuvants alone (mock-immunized).

FIG. 13B shows the gating strategy used to characterize spleen-derived CD8+ T cells. Lymphocytes were identified by a low forward scatter (FSC) and low side scatter (SSC) gate. Singlets were selected by plotting forward scatter area (FSC-A) vs. forward scatter height (FSC-H). CD8 positive cells were then gated by the expression of CD8 and CD3 markers.

FIG. 13C shows a representative ELISpot image (left panel) and average frequencies (right panel) of IFN-γ-producing cell spots from splenocytes (106 cells/well) stimulated for 48 hours with 10 μM of 10 immunodominant CD8+ T cell peptides and 1 subdominant CD8+ T cell peptide out of the total pool of 27 CD8+ T cell peptides derived from SARS-CoV-2 structural and non-structural proteins. The number on the top of each ELISpot image represents the number of IFN-γ-producing spot forming T cells (SFC) per one million splenocytes.

FIG. 13D shows a representative FACS plot (left panel) and average frequencies (right panel) of IFN-γ and TNF-production by, and CD107a/b and CD69 expression on 10 immunodominant CD8+ T cell peptides and 1 subdominant CD8+ T cell peptide out of the total pool of 27 CD8+ T cell peptides derived from SARS-CoV-2 structural and non-structural proteins determined by FACS. Numbers indicate frequencies of IFN-γ+CD8+ T cells, CD107+CD8+ T cells, CD69+CD8+ T cells and TNF-+CD8+ T cells, detected in 3 immunized mice.

FIG. 14 shows the SARS-CoV/SARS-CoV-2 genome encodes two large non-structural genes ORF1a (green) and ORF1b (gray), encoding 16 non-structural proteins (NSP1-NSP16). The genome encodes at least six accessory proteins (shades of light grey) that are unique to SARS-CoV/SARS-CoV-2 in terms of number, genomic organization, sequence, and function. The common SARS-CoV, SARS-CoV-2 and SL-CoVs-derived human B (blue); CD4+ (green) and CD8+ (black) T cell epitopes are shown. Structural and non-structural open reading frames utilized in this study were from SARS-CoV-2-Wuhan-Hu-1 strain (NCBI accession number MN908947.3, SEQ ID NO: 1). The amino acid sequence of the SARS-CoV-2-Wuhan-Hu-1 structural and non-structural proteins was screened for human B, CD4+ and CD8+ T cell epitopes using different computational algorithms as described herein. Shown are genome-wide identified SARS-CoV-2 human B cell epitopes (in blue), CD4+ T cell epitopes (in green), CD8+ T cell epitopes (in black) that are highly conserved between human and animal Coronaviruses.

FIG. 15 shows the identification of highly conserved potential SARS-CoV-2-derived human CD4+ T cell epitopes that bind with high affinity to HLA-DR molecules: Out of a total of 9,594 potential HLA-DR-restricted CD4+ T cell epitopes from the whole genome sequence of SARS-CoV-2-Wuhan-Hu-1 strain (MN908947.3), 16 epitopes that bind with high affinity to HLA-DRB1 molecules were selected. The conservancy of the 16 CD4+ T cell epitopes was analyzed among human and animal Coronaviruses. Shown are the comparison of sequence homology for the 16 CD4+ T cell epitopes among 81,963 SARS-CoV-2 strains (that currently circulate in 6 continents), the 4 major “common cold” Coronaviruses that cased previous outbreaks (i.e. hCoV-OC43, hCoV-229E, hCoV-HKU1, and hCoV-NL63), and the SL-CoVs that were isolated from bats, civet cats, pangolins and camels. Epitope sequences highlighted in green present high degree of homology among the currently circulating 81,963 SARS-CoV-2 strains and at least a 50% conservancy among two or more humans SARS-CoV strains from previous outbreaks, and the SL-CoV strains isolated from bats, civet cats, pangolins and camels, as described in Materials and Methods. Homo Sapiens—black, bats (Rhinolophus affinis, Rhinolophus malayanus—red), pangolins (Manis javanica—blue), civet cats (Paguma larvata—green), and camels (Camelus dromedaries—brown).

FIG. 16A the molecular docking of highly conserved SARS-CoV-2 CD4+ T cell epitopes to HLA-DRBI molecules. Molecular docking of 16 CD4+ T cell epitopes, conserved among human SARS-CoV-2 strains, previous humans SARS/MERS-CoV and bat SL-CoVs into the groove of the HLA-DRB1 protein crystal structure (PDB accession no: 4UQ3) was determined using the GalaxyPepDock server. The 16 CD4+ T cell epitopes are promiscuous restricted to HLA-DRB1*01:01, HLA-DRB1*11:01, HLA-DRB1*15:01, HLA-DRB1*03:01 and HLA-DRB1*04:01 alleles. The CD4+ T cell peptides are shown in ball and stick structures, and the HLA-DRB1 protein crystal structure is shown as a template. The prediction accuracy is estimated from a linear model as the relationship between the fraction of correctly predicted binding site residues and the template-target similarity measured by the protein structure similarity score (TM score) and interaction similarity score (Sinter) obtained by linear regression. Sinter shows the similarity of the amino acids of the CD8+ T cell peptides aligned to the contacting residues in the amino acids of the HLA-DRB1 template structure.

FIG. 16B shows histograms representing interaction similarity score of CD4+ T cells specific epitopes observed from the protein-peptide molecular docking analysis.

FIG. 17A shows an experimental design to show CD4+ T cells are specific to highly conserved SARS-CoV-2 epitopes detected in COVID-19 patients and unexposed healthy individuals: PBMCs from HLA-DRB1 positive COVID-19 patients (n=30) and controls unexposed healthy individuals (n=10) were isolated and stimulated for 48 hrs. with 10 μM of each of the 16 SARS-CoV-2-derived CD4+ T cell epitopes. The number of IFN-producing cells were quantified using ELISpot assay.

FIG. 17B shows the results from FIG. 17A. Dotted lines represent a threshold to evaluate the relative magnitude of the response: a mean SFCs between 25 and 50 correspond to a medium/intermediate response, whereas a strong response is defined for a mean SFCs >50. PBMCs from HLA-DRB1-positive COVID-19 patients

FIG. 17C shows the results from further stimulating for an additional 5 hours in the presence of mAbs specific to CD107a and CD107b, and Golgi-plug and Golgi-stop. Tetramers specific to two Spike epitopes, CD107a/b and CD69 and TNF-alpha expression were then measured by FACS. Representative FACS plot showing the frequencies of Tetramer+CD4+ T cells, CD107a/b+CD4+ T cells, CD69+CD4+ T cells and TNF-+CD4+ T cells following priming with a group of 2 Spike CD4+ T cell epitope peptides. Average frequencies are shown for tetramer+CD4+ T cells, CD107a/b+CD4+ T cells, CD69+CD4+ T cells and TNF-+CD4+ T cells.

FIG. 18A shows a timeline of immunization and immunological analyses for testing immunogenicity of genome-wide identified human SARS-CoV-2 CD4.+ T epitopes in HLA-A*02:01/HLA-DRB1 double transgenic mice. Four groups of age-matched HLA-DRB1 transgenic mice (n=3) were immunized subcutaneously, on days 0 and 14, with a mixture of four SARS-CoV-2-derived human CD4+ T cell peptide epitopes delivered in alum and CpG1826 adjuvants. As a negative control, mice received adjuvants alone (mock-immunized).

FIG. 18B shows the gating strategy used to characterize spleen-derived CD4+ T cells. CD4 positive cells were gated by the CD4 and CD3 expression markers.

FIG. 18C shows the representative ELISpot images (left panel) and average frequencies (right panel) of IFN-γ-producing cell spots from splenocytes (106 cells/well) stimulated for 48 hours with 10 μM of 7 immunodominant CD4+ T cell peptides and 1 subdominant CD4+ T cell peptide out of the total pool of 16 CD4+ T cell peptides derived from SARS-CoV-2 structural and non-structural proteins. The number of IFN-γ-producing spot forming T cells (SFC) per one million of total cells is presented on the top of each ELISpot image.

FIG. 18D shows the representative FACS plot (left panel) and average frequencies (right panel) show IFN-γ and TNF-α-production by, and CD107a/b and CD69 expression on 7 immunodominant CD4+ T cell peptides and 1 subdominant CD4+ T cell peptide out of the total pool of 16 CD4+ T cell peptides derived from SARS-CoV-2 determined by FACS. The numbers indicate percentages of IFN-γ+CD4+ T cells, CD107+CD4+ T cells, CD69+CD4+ T cells and TNF-α+CD4+ T cells detected in 3 immunized mice.

FIG. 19 shows the conservation of Spike-derived B cell epitopes among human, bat, civet cat, pangolin, and camel coronavirus strains: Multiple sequence alignment performed using ClustalW among 29 strains of SARS coronavirus (SARS-CoV) obtained from human, bat, civet, pangolin, and camel. This includes 7 human SARS/MERS-CoV strains (SARS-CoV-2-Wuhan (MN908947.3), SARS-HCoV-Urbani (AY278741.1), CoV-HKU1-Genotype-B (AY884001), CoV-OC43 (KF923903), CoV-NL63 (NC005831), CoV-229E (KY983587), MERS (NC019843)); 8 bat SARS-CoV strains (BAT-SL-CoV-WIV16 (KT444582), BAT-SL-CoV-WIV1 (KF367457.1), BAT-SL-CoV-YNLF31C (KP886808.1), BAT-SARS-CoV-RS672 (FJ588686.1), BAT-CoV-RATG13 (MN996532.1), BAT-CoV-YN01 (EPIISL412976), BAT-CoV-YNO2 (EPIISL412977), BAT-CoV-19-ZXC21 (MG772934.1); 3 Civet SARS-CoV strains (SARS-CoV-Civet007 (AY572034.1), SARS-CoV-A022 (AY686863.1), SARS-CoV-B039 (AY686864.1)); 9 pangolin SARS-CoV strains (PCoV-GX-P2V (MT072864.1), PCoV-GX-P5E (MT040336.1), PCoV-GX-P5L (MT040335.1), PCoV-GX-P1E (MT040334.1), PCoV-GX-P4L (MT040333.1), PCoV-MP789 (MT084071.1), PCoV-GX-P3B (MT072865.1), PCoV-Guangdong-P2S (EPIISL410544), PCoV-Guangdong (EPHSL410721)); 4 camel SARS-CoV strains (Camel-CoV-HKU23 (KT368891.1), DcCoV-HKU23 (MN514967.1), MERS-CoV-Jeddah (KF917527.1), Riyadh/RY141 (NC028752.1)) and 1 recombinant strain (FJ211859.1)). Regions highlighted with blue color represent the sequence homology. The B cell epitopes, which showed at least 50% conservancy among two or more strains of the SARS Coronavirus or possess receptor-binding domain (RBD) specific amino acids were selected as candidate epitopes.

FIG. 20A shows the docking of SARS-CoV-2 Spike glycoprotein-derived B cell epitopes to human ACE2 receptor, e.g., molecular docking of 22 B-cell epitopes, identified from the SARS-CoV-2 Spike glycoprotein, with ACE2 receptors. B cell epitope peptides are shown in ball and stick structures whereas the ACE2 receptor protein is shown as a template. S471-501 and S369-393 peptide epitopes possess receptor binding domain region specific amino acid residues. The prediction accuracy is estimated from a linear model as the relationship between the fraction of correctly predicted binding site residues and the template-target similarity measured by the protein structure similarity score and interaction similarity score (Sinter) obtained by linear regression. Sinter shows the similarity of amino acids of the B-cell peptides aligned to the contacting residues in the amino acids of the ACE2 template structure. Higher Sinter score represents a more significant binding affinity among the ACE2 molecule and B-cell peptides.

FIG. 20B shows the summary of the interaction similarity score of 22 B cells specific epitopes observed from the protein-peptide molecular docking analysis. B cell epitopes with high interaction similarity scores are indicated in black.

FIG. 21A shows the timeline of immunization and immunological analyses for testing to show IgG antibodies are specific to SARS-CoV-2 Spike protein-derived B-cell epitopes in immunized B6 mice and in convalescent COVID-19 patients. A total of 22 SARS-CoV-2 derived B-cell epitope peptides selected from SARS-CoV-2 Spike protein and tested in B6 mice were able to induce antibody responses. Four groups of age-matched B6 mice (n=3) were immunized subcutaneously, on days 0 and 14, with a mixture of 4 or 5 SARS-CoV-2 derived B-cell peptide epitopes emulsified in alum and CpG1826 adjuvants. AlumCpG1826 adjuvants alone were used as negative controls (mock-immunized).

FIG. 21B shows the frequencies of IgG-producing CD3(−)CD138(+)B220(+) plasma B cells were determined in the spleen of immunized mice by flow cytometry. For example, FIG. 21B shows the gating strategy was as follows: Lymphocytes were identified by a low forward scatter (FSC) and low side scatter (SSC) gate. Singlets were selected by plotting forward scatter area (FSC-A) versus forward scatter height (FSC-H). B cells were then gated by the expression of CD3(−) and B220(+) cells and CD138 expression on plasma B cells determined.

FIG. 21C shows the frequencies of IgG-producing CD3(−)CD138(+)B220(+) plasma B cells were determined in the spleen of immunized mice by flow cytometry. For example, FG 15C shows a representative FACS plot (left panels) and average frequencies (right panel) of plasma B cells detected in spleen of immunized mice. The percentages of plasma CD138(−)B220(+)B cells are indicated on the top left of each dot plot,

FIG. 21D shows SARS-CoV-2 derived B-cell epitopes-specific IgG responses were quantified in immune serum, 14 days post-second immunization (i.e. day 28), by ELISpot (Number of IgG(+)Spots). Representative ELISpot images (left panels) and average frequencies (right panel) of anti-peptide specific IgG-producing B cell spots (1×106 splenocytes/well) following 4 days in vitro B cell polyclonal stimulation with mouse Poly-S (Immunospot). The top/left of each ELISpot image shows the number of IgG-producing B cells per half a million cells. ELISA plates were coated with each individual immunizing peptide.

FIG. 21E shows the B-cell epitopes-specific IgG concentrations (μg/mL) measured by ELISA in levels of IgG detected in peptide-immunized 86 mice, after subtraction of the background measured from mock-vaccinated mice. The dashed horizontal line indicates the limit of detection.

FIG. 21F and FIG. 21G show the B-cell epitopes-specific IgG concentrations (μg/mL) measured by ELISA in Level of IgG specific to each of the 22 Spike peptides detected SARS-CoV-2 infected patients (n=40), after subtraction of the background measured from healthy non-exposed individuals (n=10). Black bars and gray bars show high and medium immunogenic B cell peptides, respectively. The dashed horizontal line indicates the limit of detection.

FIG. 22 shows an example of a whole spike protein comprising mutations including 6 proline mutations. The 6 proline mutations comprise single point mutations F817P, A892P, A899P, A942P, K986P and V987P. Additionally, the spike protein comprises a 682-QQAQ-685 mutation of the furin cleavage site for protease resistance. In some embodiments, the K986P and V987P Mutations allow for perfusion stabilization. FIG. 22 also shows the following sequences: MFVFLVLLPLVSS (SEQ ID NO: 63), ATGTTCGTGTTCCTGGTGCTGCTGCCCCTGGTGAGCAGC (SEQ ID NO: 422), CAGCAGGCCCAG (SEQ ID NO: 423), and CCCCCC.

FIG. 23 shows non-limiting examples of how the large sequences of the compositions described herein may be arranged.

FIG. 24 shows a schematic representation of a prototype Coronavirus vaccine of the present invention. The present invention is not limited to the prototype coronavirus vaccines as shown.

FIG. 25A shows a non-limiting example of a method for delivering the vaccine composition described herein using a “prime/pull” regimen in humans. The method comprises administering a pan-coronavirus recombinant vaccine composition and further administering at least one T-cell attracting chemokine (e.g. CXCL11) after administering the pan-coronavirus recombinant vaccine composition.

FIG. 25B shows a non-limiting example of a method for delivering the vaccine composition described herein using a “prime/boost” regimen in humans. The method comprises administering a first composition, e.g., a first pan-coronavirus recombinant vaccine composition dose using a first delivery system and further administering a second composition, e.g., a second vaccine composition dose using a second delivery system. In some embodiments, the first delivery system and the second delivery system are different.

FIG. 25C shows a non-limiting example of a method for delivering the vaccine composition described herein using a “prime/pull/keep” regimen in humans to increase the size and maintenance of lung-resident B-cells, CD4+ T cells and CD8+ T cells to protect against SARS-CoV-2. The method comprises administering a pan-coronavirus recombinant vaccine composition and administering at least one T-cell attracting chemokine (e.g. CXCL11 or CXCL17) after administering the pan-coronavirus recombinant vaccine composition.

FIG. 25D shows a non-limiting example of a method for delivering the vaccine composition described herein using a “prime/pull/boost” regimen in humans to increase the size and maintenance of lung-resident B-cells, CD4+ T cells and CD8+ T cells to protect against SARS-CoV-2. The method comprises administering a pan-coronavirus recombinant vaccine composition and administering at least one T-cell attracting chemokine (e.g. CXCL11 or CXCL17) after administering the pan-coronavirus recombinant vaccine composition. The method further comprises administering at least one cytokine after administering the T-cell attracting chemokine (e.g. IL-7, IL-5, or IL-2).

FIG. 26A shows a non-limiting example of a method for delivering the vaccine composition described herein using a “prime/pull” regimen in domestic animals (e.g. cats or dogs). The method comprises administering a pan-coronavirus recombinant vaccine composition and further administering at least one T-cell attracting chemokine (e.g. CXCL11) after administering the pan-coronavirus recombinant vaccine composition.

FIG. 26B shows a non-limiting example of a method for delivering the vaccine composition described herein using a “prime/boost” regimen in domestic animals (e.g. cats or dogs). The method comprises administering a first composition, e.g., a first pan-coronavirus recombinant vaccine composition dose using a first delivery system and further administering a second composition, e.g., a second vaccine composition dose using a second delivery system. In some embodiments, the first delivery system and the second delivery system are different.

FIG. 26C shows a non-limiting example of a method for delivering the vaccine composition described herein using a “prime/pull/keep” regimen in domestic animals (e.g. cats or dogs) to increase the size and maintenance of lung-resident B-cells, CD4+ T cells and CD8+ T cells to protect against SARS-CoV-2. The method comprises administering a pan-coronavirus recombinant vaccine composition and administering at least one T-cell attracting chemokine (e.g. CXCL11 or CXCL17) after administering the pan-coronavirus recombinant vaccine composition.

FIG. 26D shows a non-limiting example of a method for delivering the vaccine composition described herein using a “prime/pull/boost” regimen in domestic animals (e.g. cats or dogs) to increase the size and maintenance of lung-resident B-cells, CD4+ T cells and CD8+ T cells to protect against SARS-CoV-2. The method comprises administering a pan-coronavirus recombinant vaccine composition and administering at least one T-cell attracting chemokine (e.g. CXCL11 or CXCL17) after administering the pan-coronavirus recombinant vaccine composition. The method further comprises administering at least one cytokine after administering the T-cell attracting chemokine (e g. IL-7, IL-5, or IL-2),

FIGS. 27A, 27B, and 27C shows experimental evidence showing immunization with pool of CD8⁺ T cell, CD4⁺ T cell, and B cell peptides to provide protection against all the current SARS-CoV-2 variants of concerns in triple transgenic h-ACE2-HLA-A2/DR mice. Viral titration (FIG. 27A), weight loss (FIG. 27B), and survival (FIG. 27C) data have been shown.

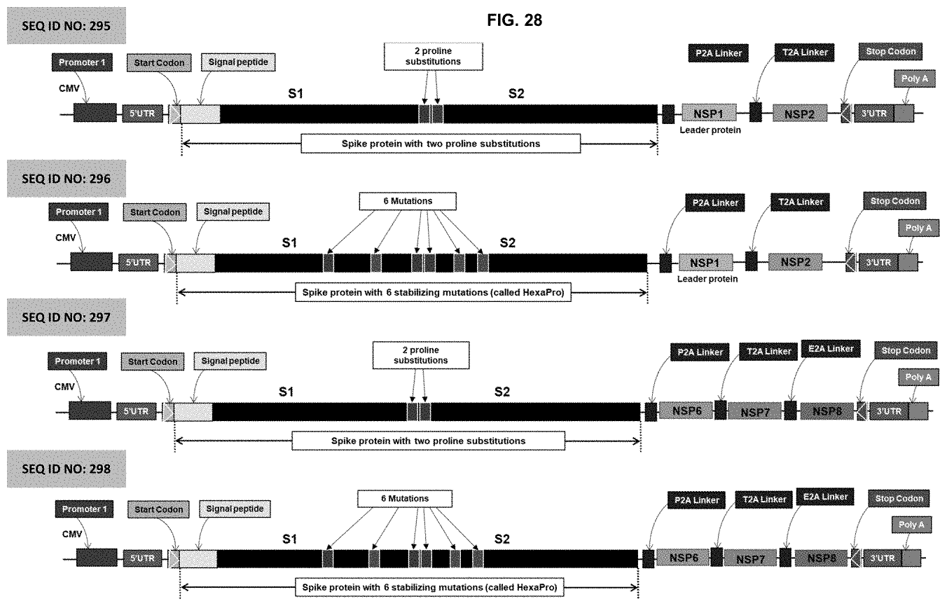

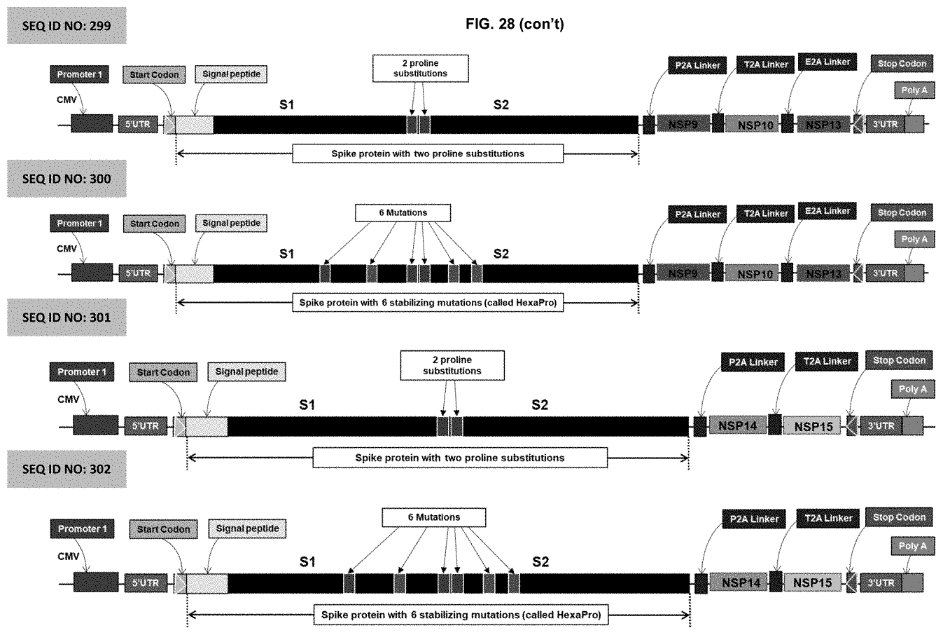

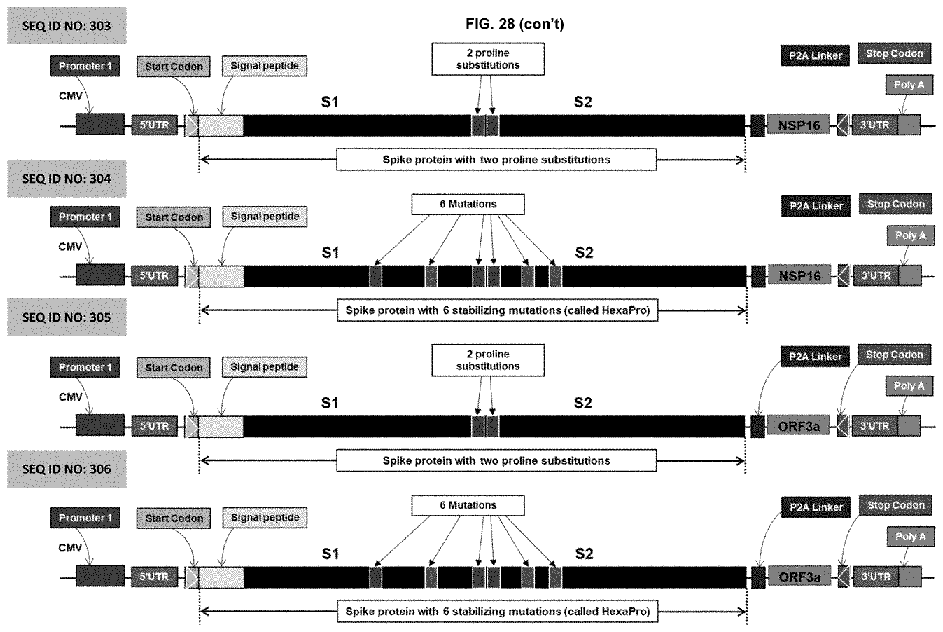

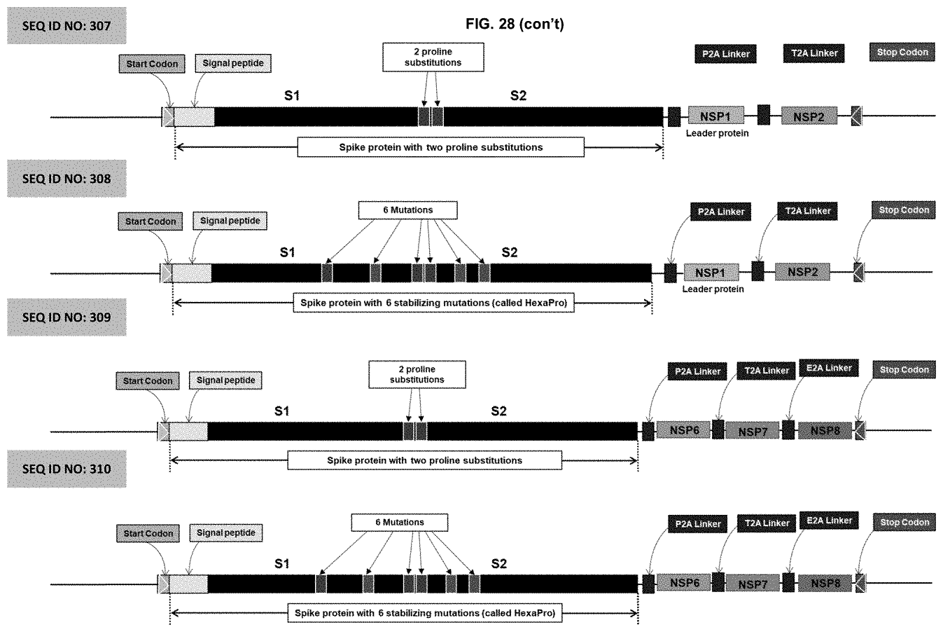

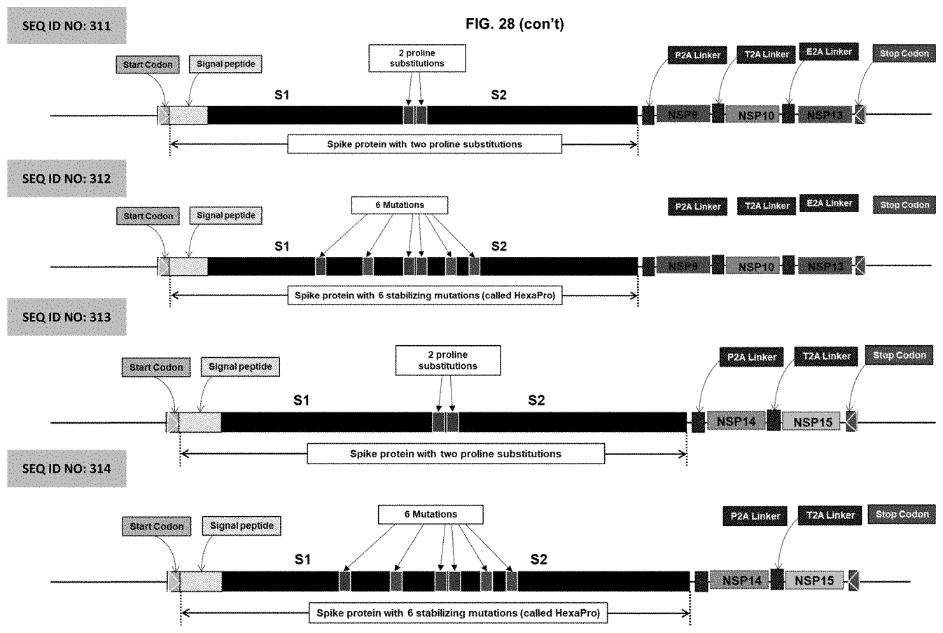

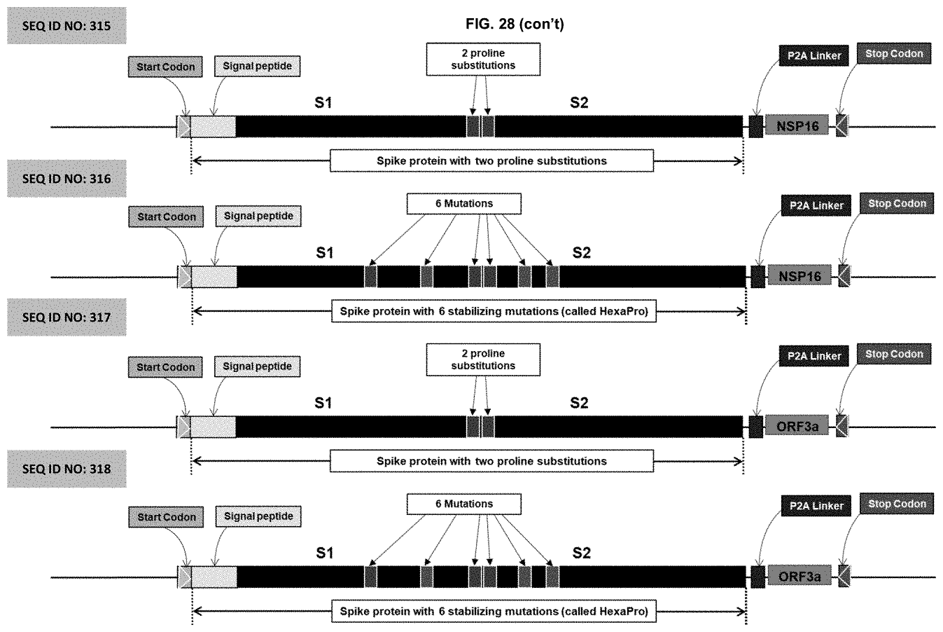

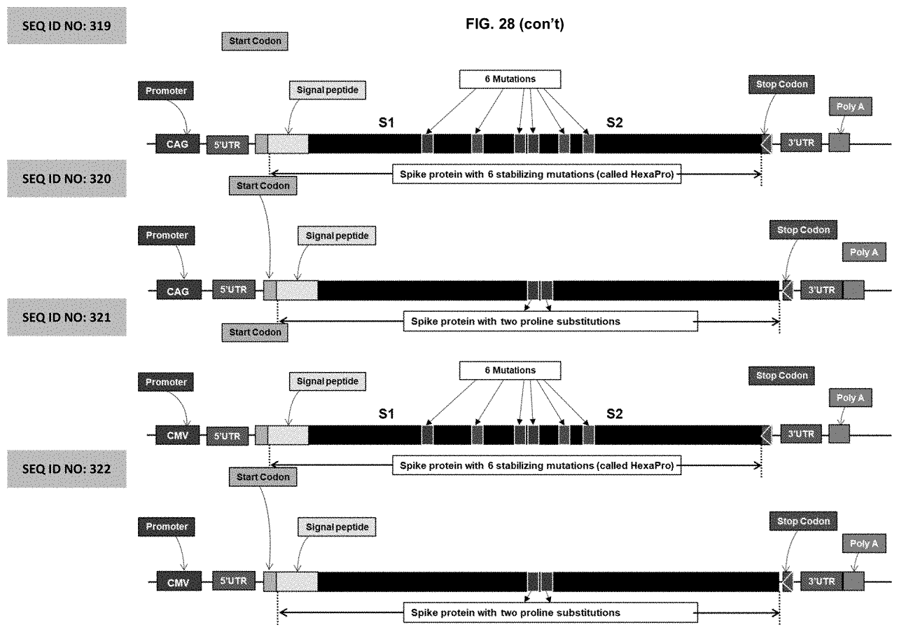

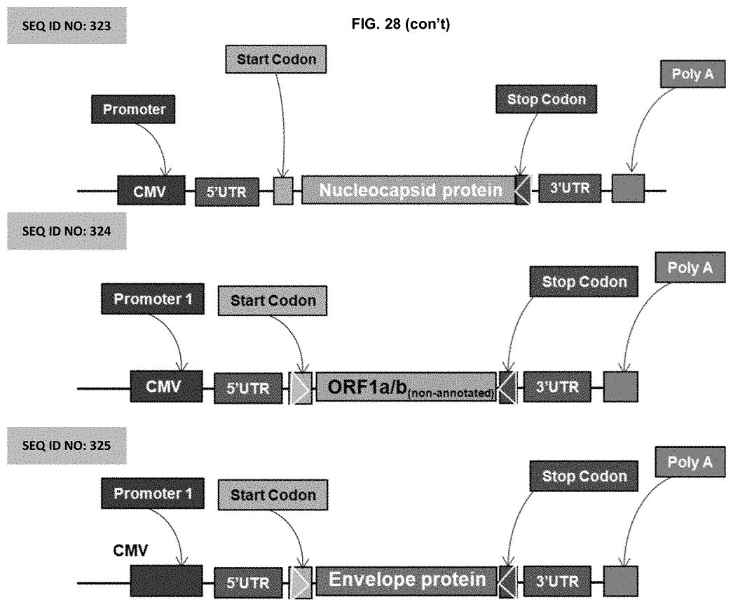

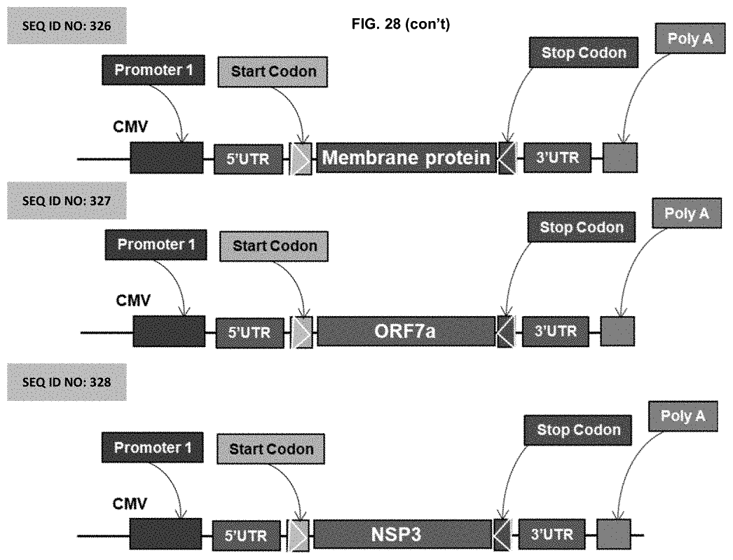

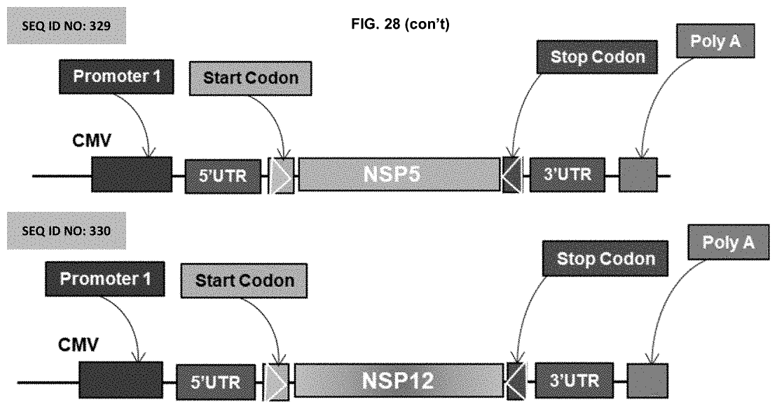

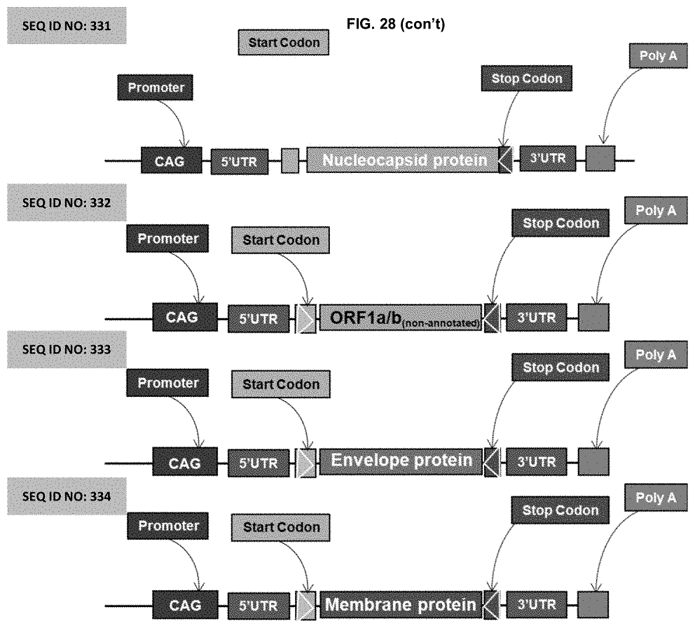

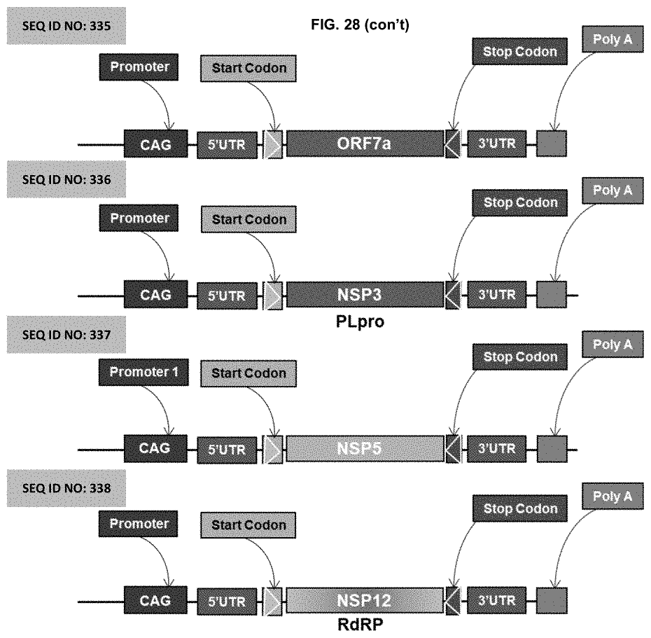

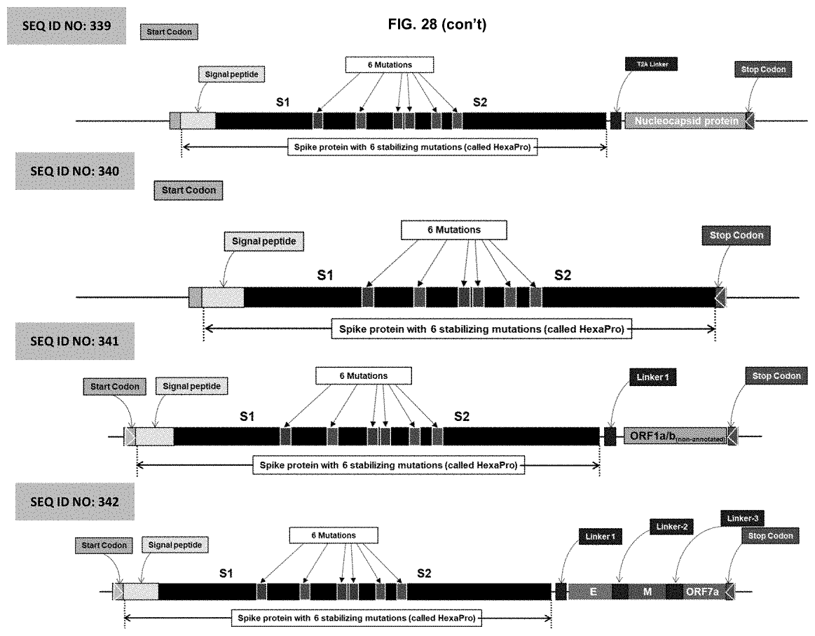

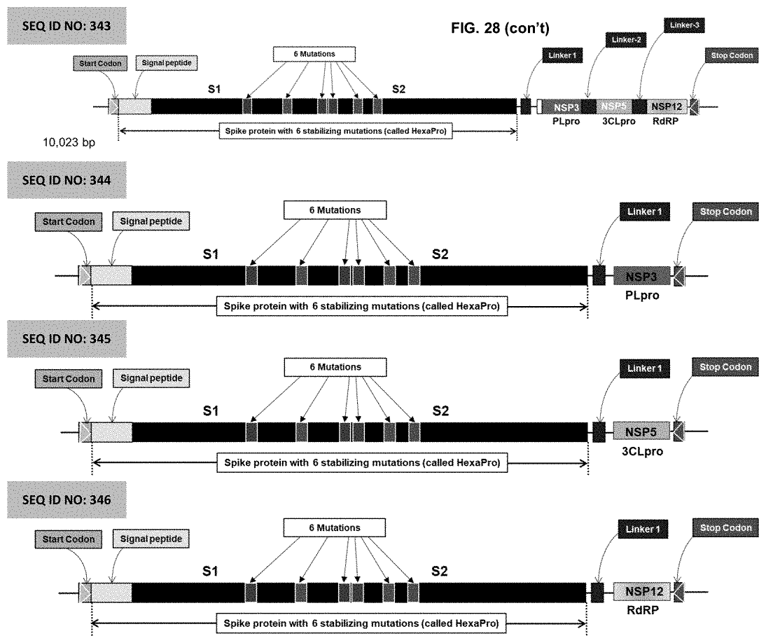

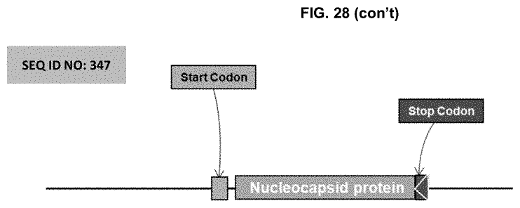

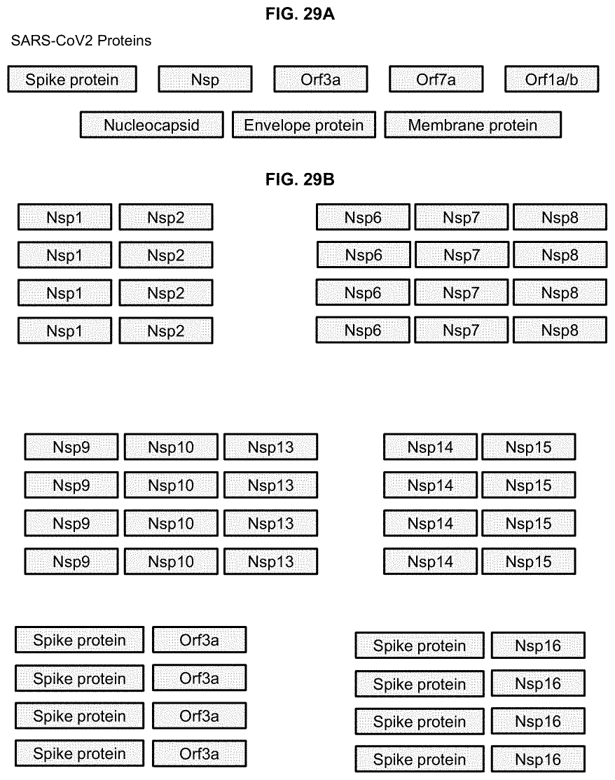

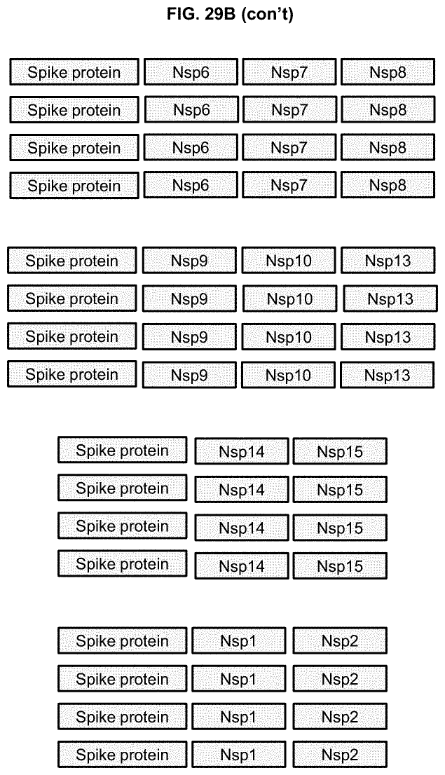

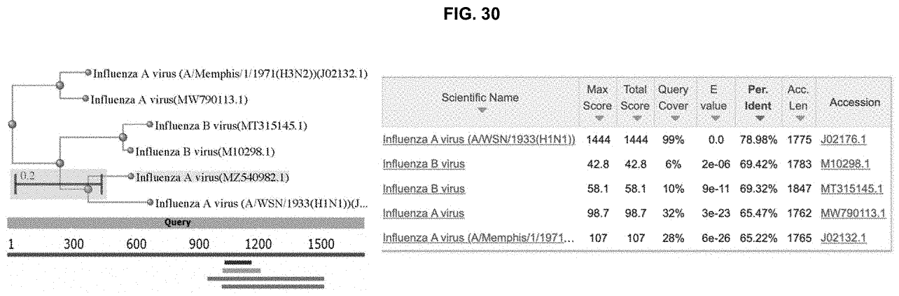

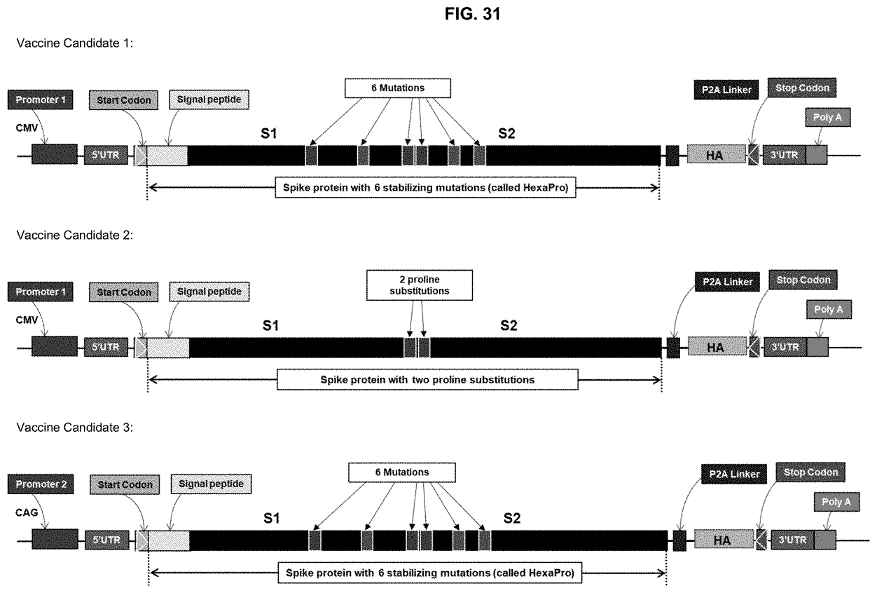

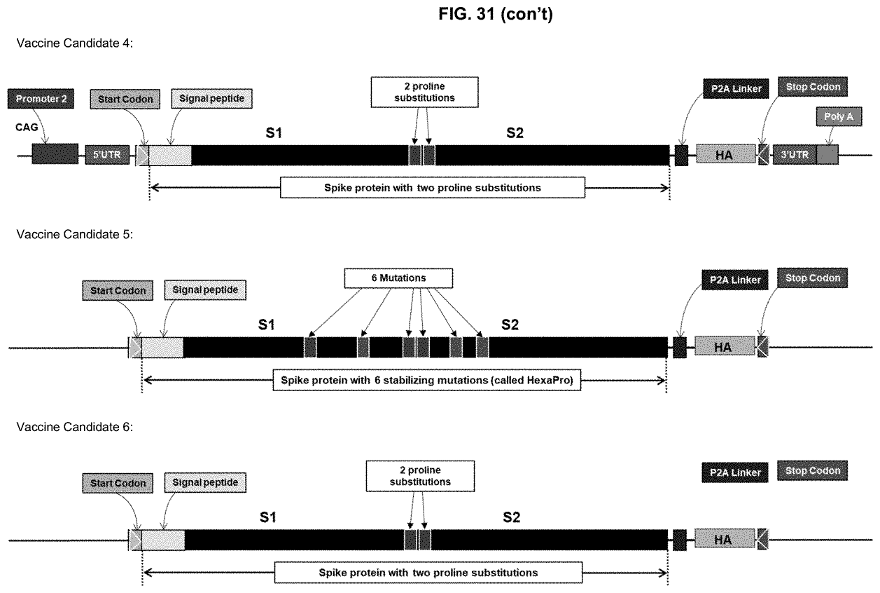

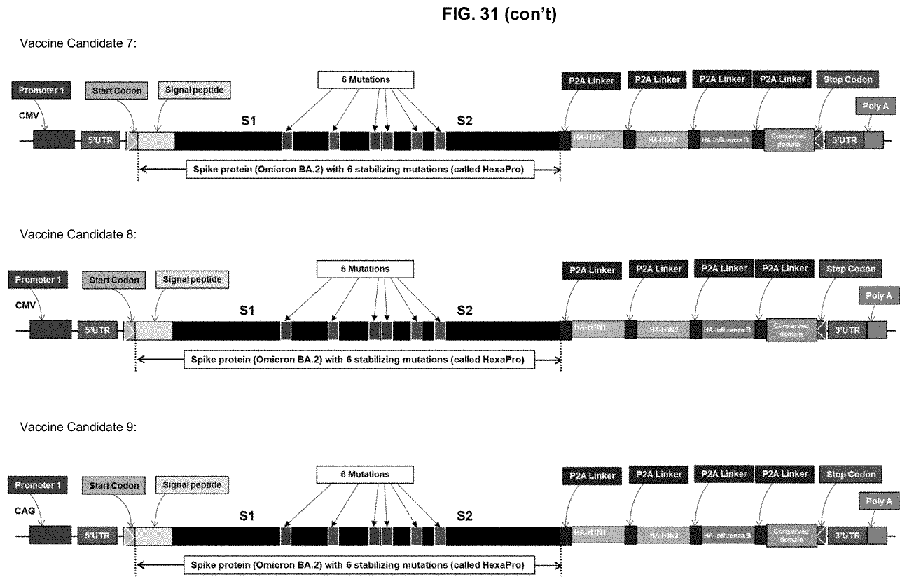

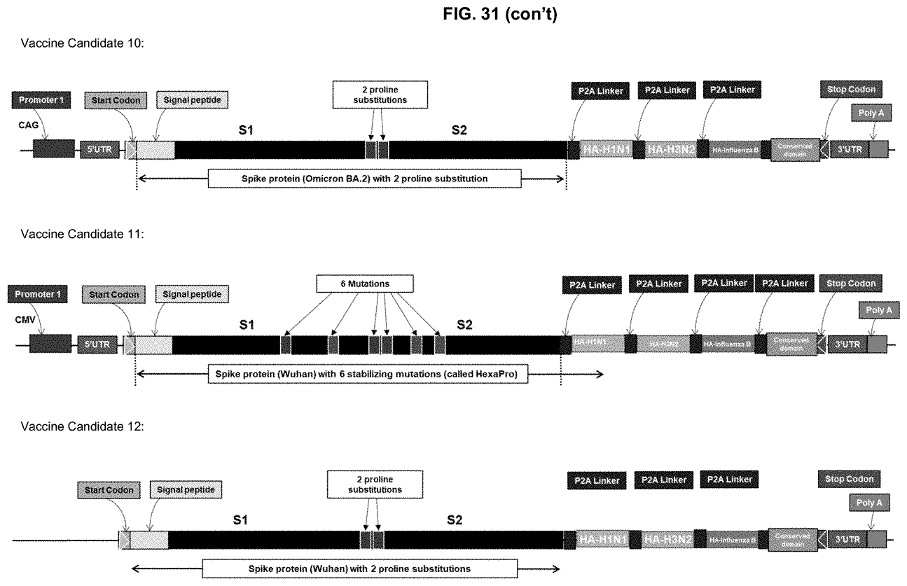

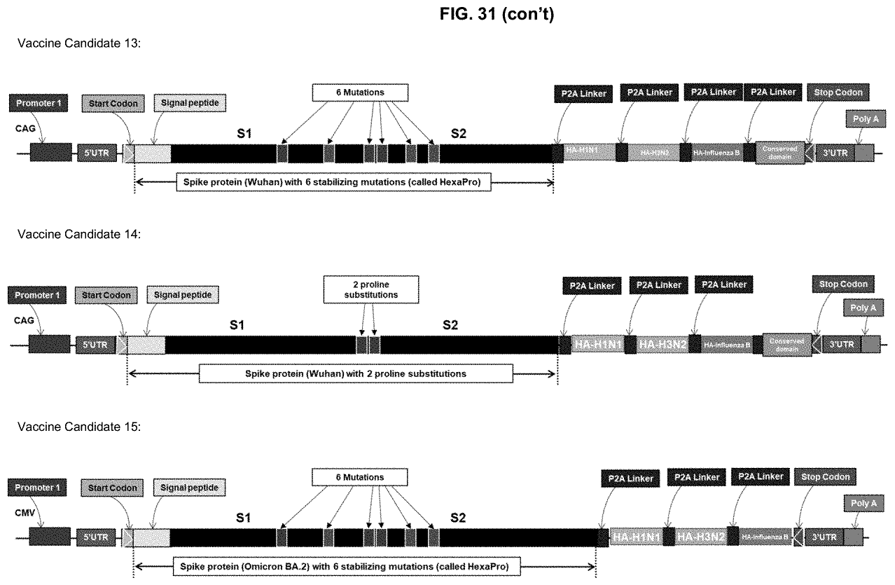

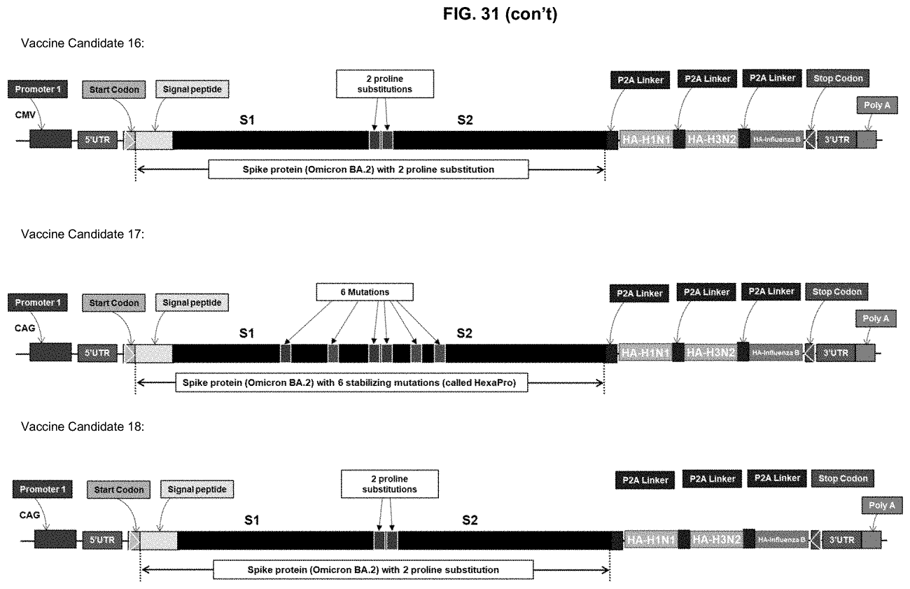

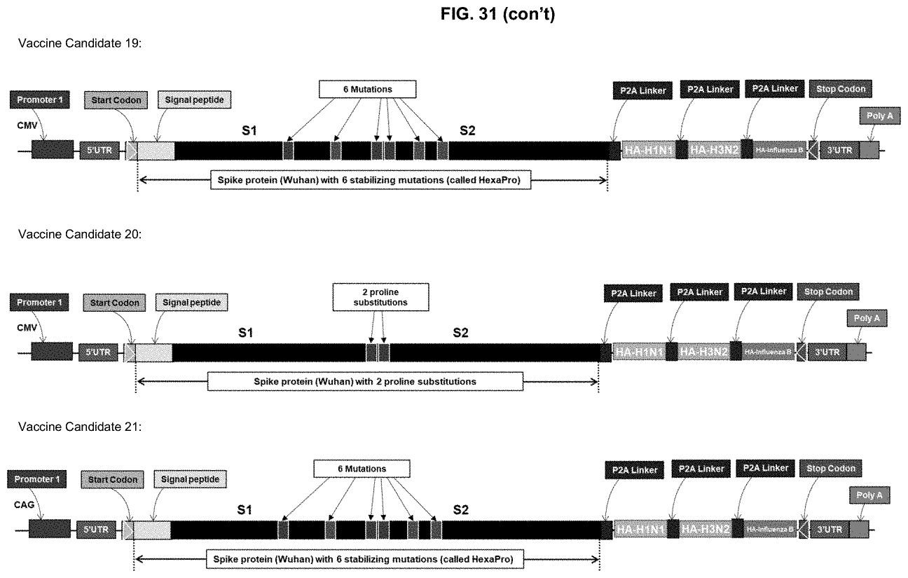

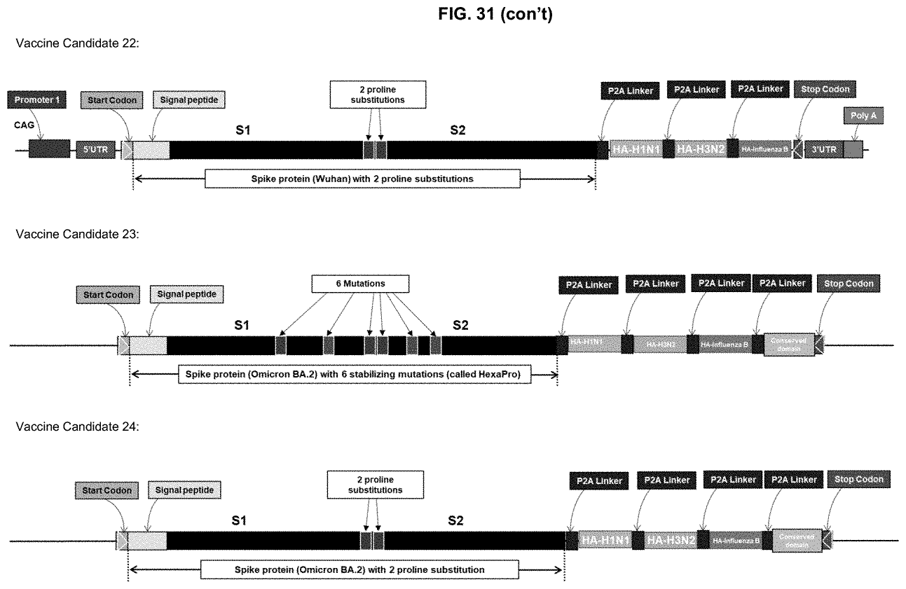

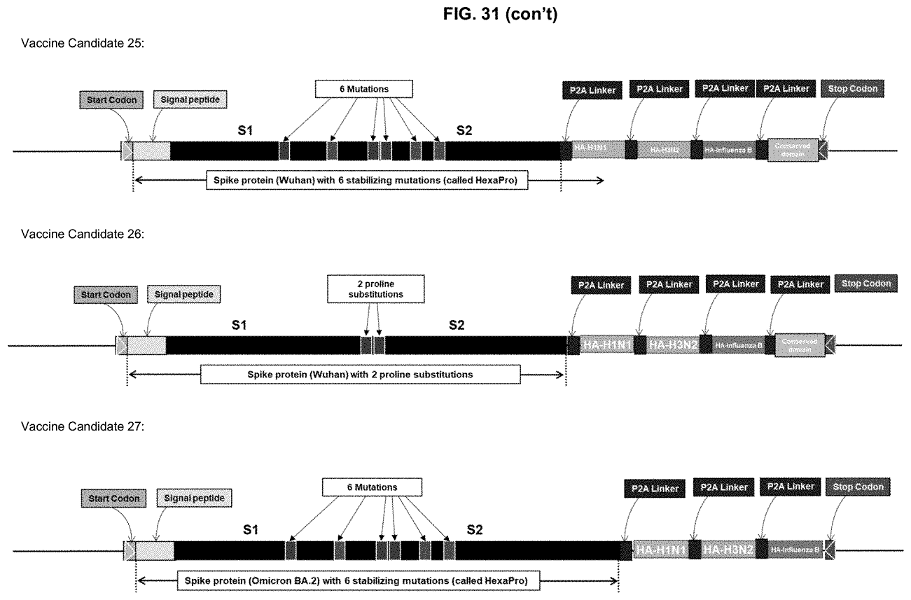

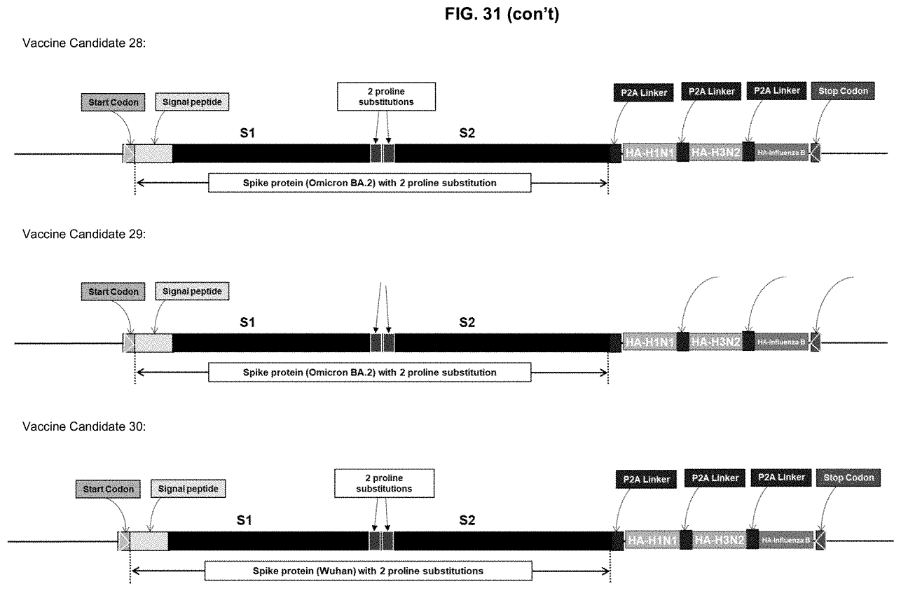

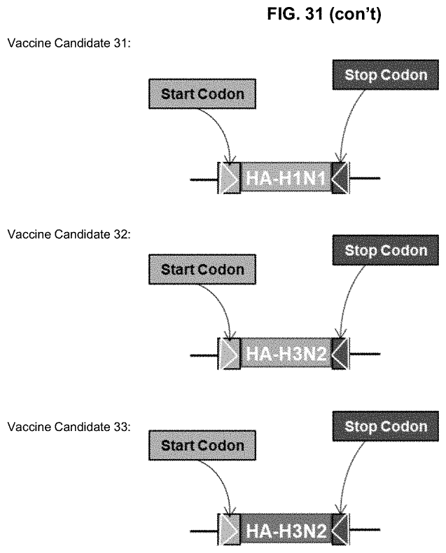

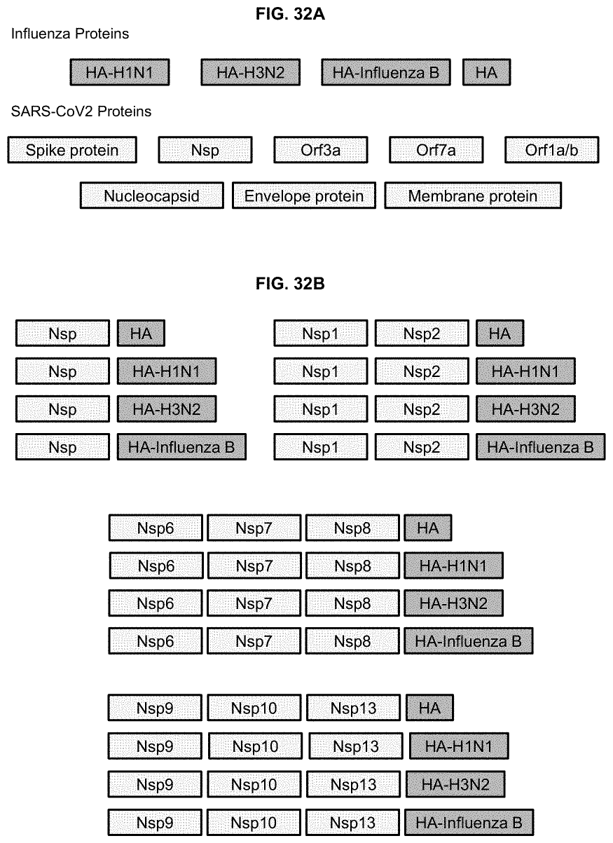

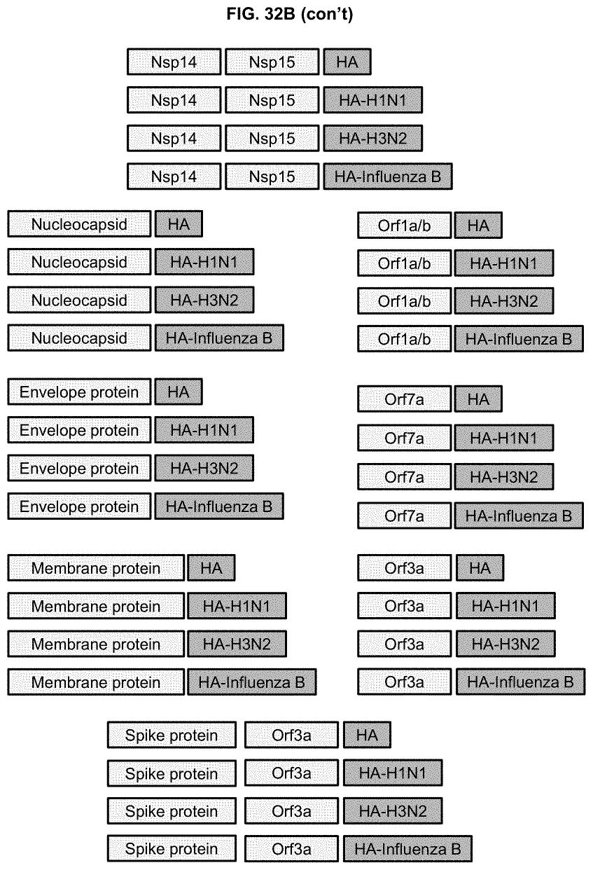

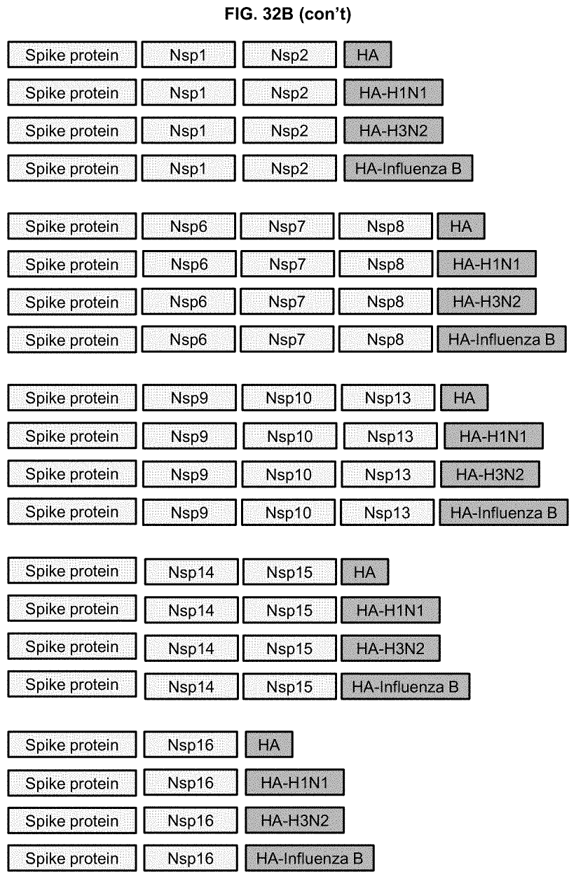

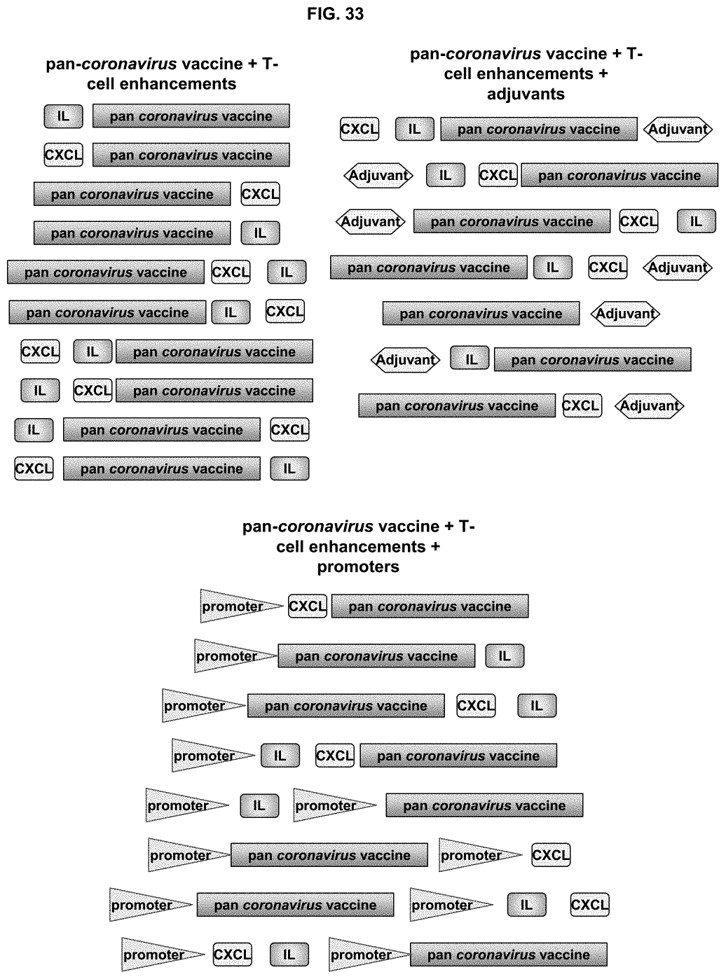

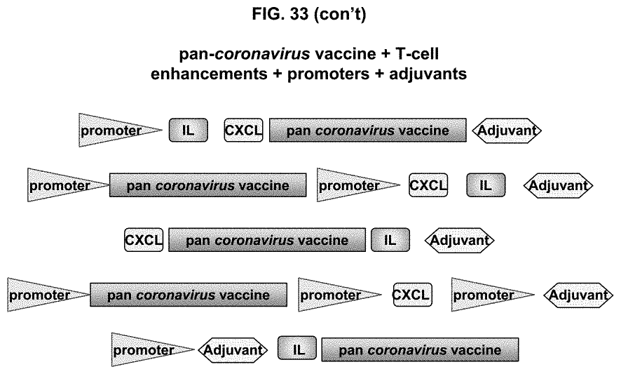

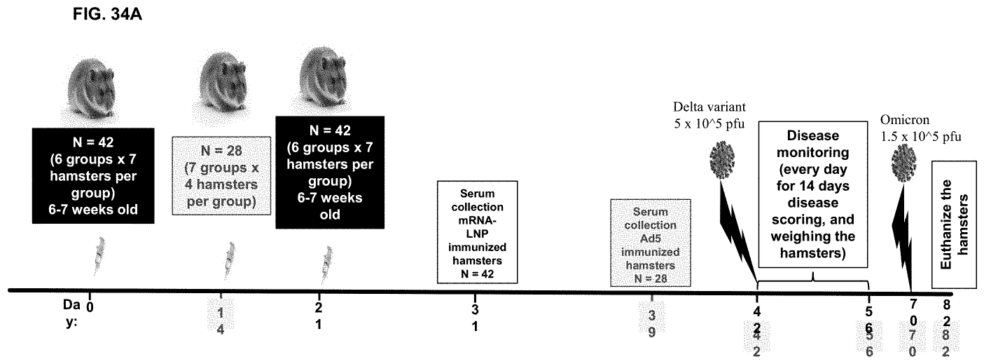

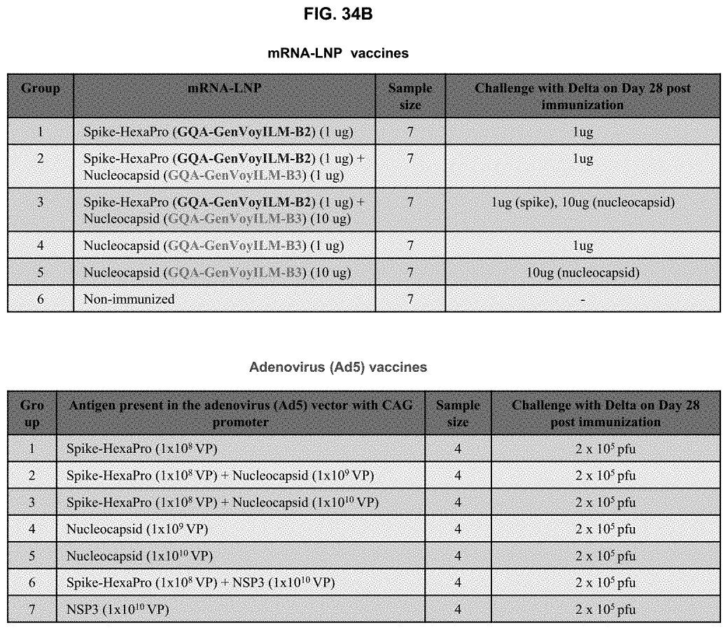

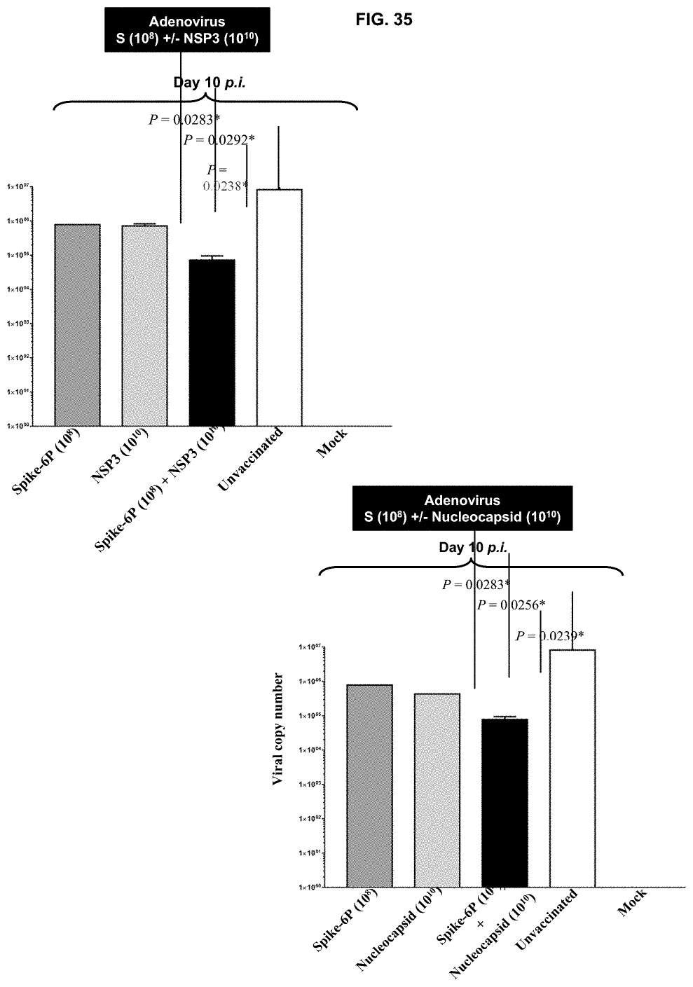

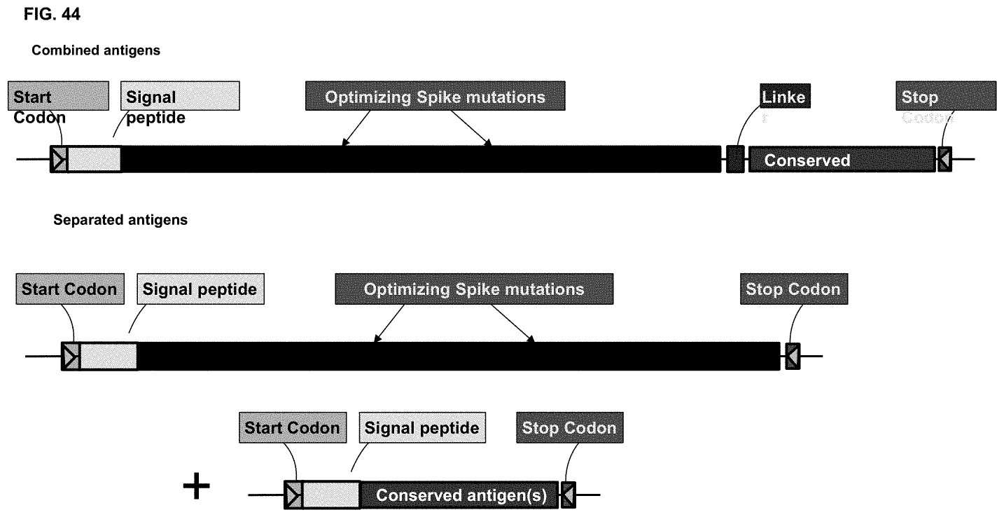

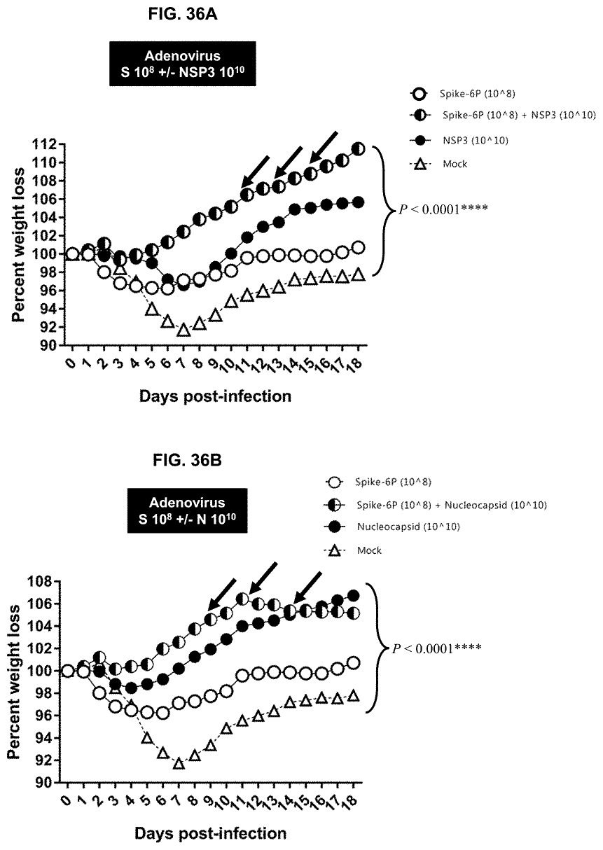

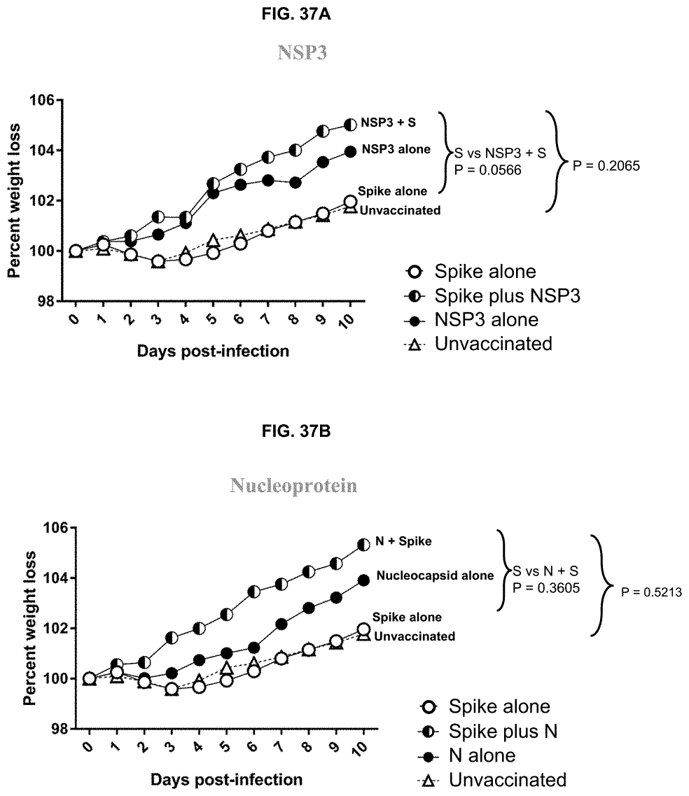

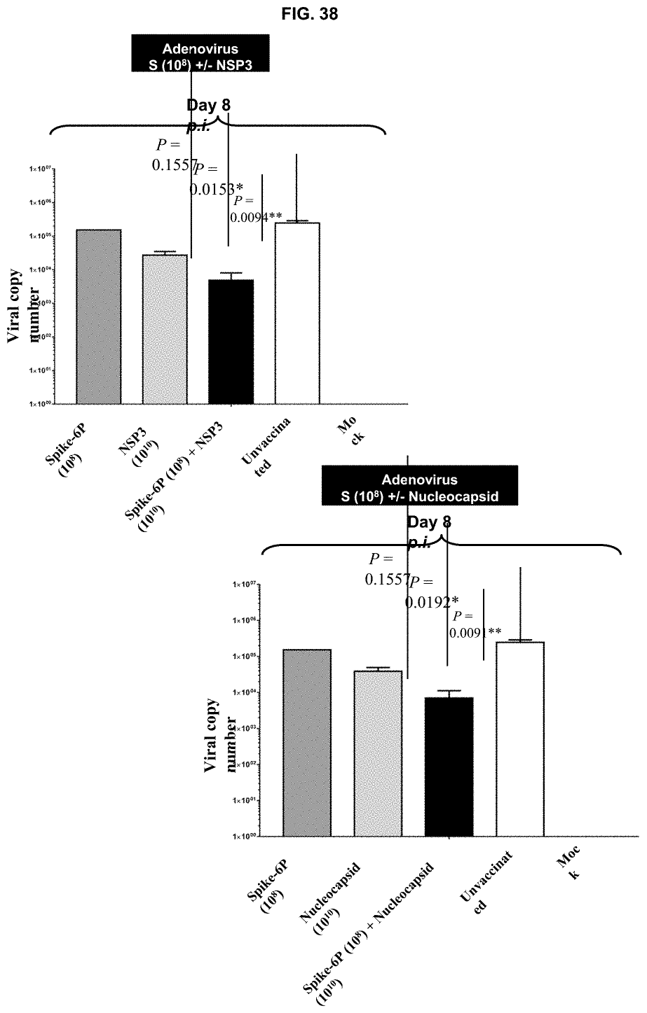

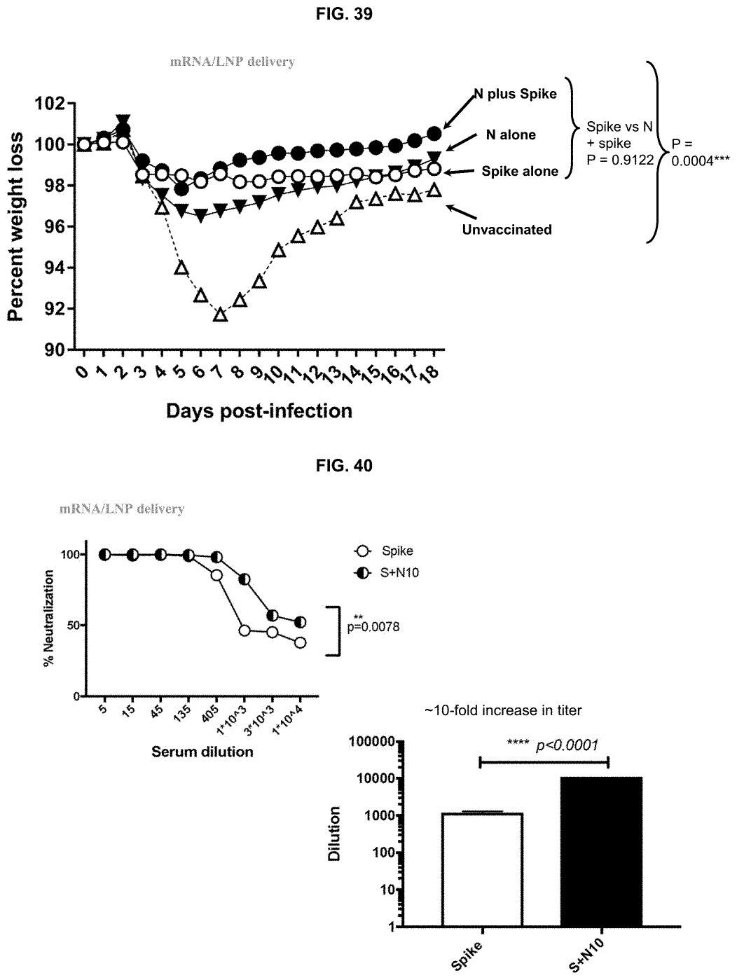

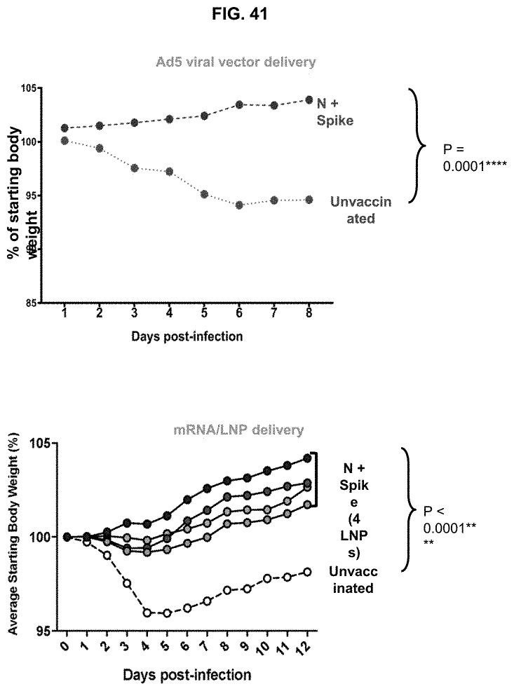

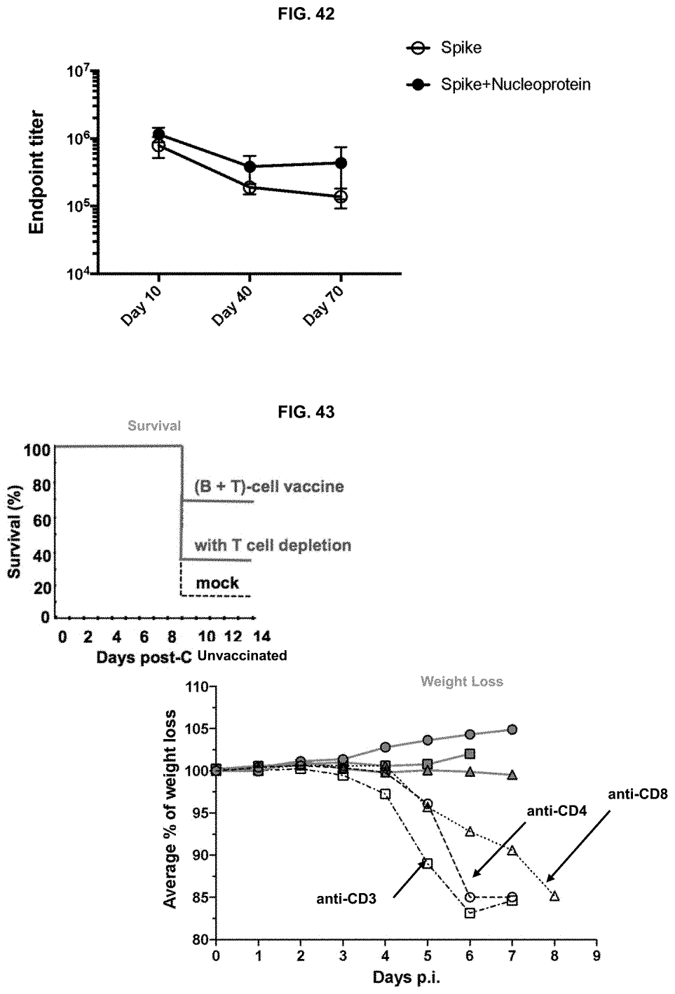

FIG. 28 shows non-limiting configurations of recombinant vaccine compositions described herein.

FIGS. 29A and 29B show non-limiting examples of recombinant hybrid vaccine compositions described herein. The proteins may be covalently or non-covalently linked together for administration of the vaccine composition. Note: “Nsp” may refer to Nsp1, Nsp2, Nsp3, Nsp5, Nsp6, Nsp7, Nsp8, Nsp9, Nsp10, Nsp12, Nsp13, Nsp14, Nsp15, Nsp16, or a combination thereof, “Spike protein” may refer to a portion of the spike protein, or a spike protein with one or more mutations (e.g., a spike protein with two or six proline substitutions).

FIG. 30 shows the results of a sequence alignment of various influenza viruses and variants and the resulting conserved region.

FIG. 31 shows non-limiting examples of recombinant vaccine compositions described herein.

FIG. 32A shows non-limiting examples of influenza proteins and SARS-CoV2 proteins that may be used in the vaccine compositions as described herein; these proteins may be used in any combination to create said compositions. Note: Nsp may refer to Nsp1, Nsp2, Nsp3, Nsp5, Nsp6, Nsp7, Nsp8, Nsp9, Nsp10, Nsp12, Nsp13. Nsp14. Nsp15, Nsp16, or a combination thereof.

FIG. 32B shows non-limiting examples of recombinant hybrid vaccine compositions described herein. The proteins may be covalently or non-covalently linked together for administration of the vaccine composition. Note: “Nsp” may refer to Nsp1, Nsp2, Nsp3, Nsp5, Nsp6, Nsp7, Nsp8, Nsp9, Nsp10, Nsp12, Nsp13, Nsp14, Nsp15, Nsp16, or a combination thereof, “Spike protein” may refer to a portion of the spike protein, or a spike protein with one or more mutations (e.g., a spike protein with two or six proline substitutions).

FIG. 33 shows schematic views of non-limiting examples of vaccine compositions showing an optional molecular adjuvant, T cell attracting chemokine, and/or composition for promoting T cell proliferation, as well as non-limiting examples of orientations of said optional molecular adjuvant, T cell attracting chemokine, and/or composition for promoting T cell proliferation.

FIG. 34A shows an experimental plan to determine the efficacy of (1) Ad5-Pan-CoV-Vaccines and (2) mRNA-LNP vaccine against the SARS-CoV-2 Delta (B.1.617.2) variant in golden hamsters. Briefly, at Day 0 the hamsters were immunized with mRNA-LNP vaccines (off the shelf LNP; volume: 50 μl); at Day 14 the hamsters were immunized intramuscularly with Ad5 vaccines (as per doses mentioned FIG. 34B: Volume: 50 μl (Ad5 vector+PBS)); at Day 21 the hamsters were immunized with mRNA-LNP vaccines (off the shelf LNP: Volume: 50 μl). For mRNA-LNP immunized hamsters: Infection 42 days after first immunization with 5×10⁵ pfu of SARS-CoV-2 B.617.2 variant (Delta) intranasally (total in both nostril) Total volume of virus given: 208 μl by the intranasal route. For Ad5 immunized hamsters: Infection 28 days after first immunization with 5×10⁵ pfu of SARS-CoV-2 B.617.2 variant (Delta) intranasally (total in both nostril) Total volume of virus given: 208 μl by the intranasal route.

FIG. 34B shows the doses given of each vaccine.

FIG. 35 de-risking of program demonstration of additive protective effect of T cells and antibodies. qRT-PCR data shows a reduction of viral copy number for hamsters immunized with Ad-5 Pan-CoV-Vaccines (i) Spike-6P (10⁸)+Nucleocapsid (10¹⁰) and (fi) Spike-6P (10⁸)+NSP3 (10¹⁰) against the SARS-CoV-2 Delta (B.1.6117.2) variant in golden hamsters.

FIGS. 36A and 36B show de-risking of program demonstration of additive protective effect of T cells and antibodies. FIG. 36A shows Physical estimation for hamsters immunized with Ad-5 Pan-CoV-Vaccines (i) Spike-6P (10⁸), (ii) Spike-61P (10⁸)+NSP3 (10¹⁰), (iii) NSP3 (10¹⁰) against the SARS-CoV-2 Delta (B.1.617.2) variant. FIG. 36B shows physical estimation for hamsters immunized with Ad-5 Pan-CoV-Vaccines (I) Spike-6P (10⁸), (ii) Spike-6P (10⁸)+Nucleocapsid (10¹⁰), (iii) Nucleocapsid (10¹⁰) against the SARS-CoV-2 Delta (B.1.617.2) variant. The arrows indicate an early additive or synergetic beneficial protective effect of adding Nucleoprotein to Spike.

FIGS. 37A and 378 show hamsters challenged with SARS-CoV-2 Delta variant then rechallenged with Omicron variant.

FIG. 38 shows qRT-PCR data demonstrating reduced viral copy number for hamsters immunized with Ad-5 Pan-CoV-Vaccines (i) Spike-6P (10⁸)+Nucleocapsid (10¹⁰) and (ii) Spike-6P (10⁸)+NSP3 (10¹⁰) against the SARS-CoV-2 Omicron variant in golden hamsters. The hamsters were immunized with Ad5 vectors and challenged initially with Delta variant on Day 28 post immunization. Subsequently the same group of animals were re-challenged with Omicron variant to see the beneficial additive or synergetic protective effect of adding Nucleoprotein to Spike.

FIG. 39 shows de-risking of program demonstration of additive protective effect of T cells and antibodies. Hamsters challenged with SARS-CoV-2 Delta variant.

FIG. 40 shows neutralizing antibodies produced against SARS-CoV-2 Delta variant.

FIG. 41 shows preclinical PoC of prototype T plus B cell SARS-CoV-2 vaccines Nucleoprotein plus Spike. Hamsters challenged with SARS-CoV-2 Washington variant.

FIG. 42 shows anti-SARS-CoV-2 Spike specific IgG measured in the serum of Hamsters immunized with, LNP encapsulated mRNA containing Spike-Hexapro alone and Spike-Hexapro mixed with Nucleoprotein on day 10, 40 and 70 after second immunization. Time kinetics based on ELISA data showing statistically significant increase in spike specific antibody in the serum of hamsters immunized with Spike-Hexapro mixed with Nucleoprotein compared to Spike-Hexapro alone. Plates were coated with 100 ng of Spike (S1+S2) from Sino biological.

FIG. 43 shows the protective role of T cells against virus. ACE-2/HLA-I/HLA-II transgenic mice were challenge 10⁴ pfu of SARS-CoV-2 Washington variant.

FIG. 44 shows non-limiting examples of how the vaccine compositions described herein may be administered.

### TERMS

Unless otherwise explained, all technical and scientific terms used herein have the same meaning as commonly understood by one of ordinary skill in the art to which a disclosed invention belongs. The singular terms “a,” “an,” and “the” include plural referents unless context clearly indicates otherwise. Similarly, the word “or” is intended to include “and” unless the context clearly indicates otherwise. The term “comprising” means that other elements can also be present in addition to the defined elements presented. The use of “comprising” indicates inclusion rather than limitation. Stated another way, the term “comprising” means “including principally, but not necessary solely”. Furthermore, variation of the word “comprising”, such as “comprise” and “comprises”, have correspondingly the same meanings. In one respect, the technology described herein related to the herein described compositions, methods, and respective component(s) thereof, as essential to the invention, yet open to the inclusion of unspecified elements, essential or not (“comprising”).

Suitable methods and materials for the practice and/or testing of embodiments of the disclosure are described below. Such methods and materials are illustrative only and are not intended to be limiting. Other methods and materials similar or equivalent to those described herein can be used. For example, conventional methods well known in the art to which the disclosure pertains are described in various general and more specific references, including, for example, Sambrook et. al., Molecular Cloning: A Laboratory Manual, 2d ed., Cold Spring Harbor Laboratory Press, 1989; Sambrook et al., Molecular Cloning: A Laboratory Manual, 3d ed., Cold Spring Harbor Press, 2001; Ausubel et al., Current Protocols in Molecular Biology, Greene Publishing Associates, 1992 (and Supplements to 2000); Ausubel et al., Short Protocols in Molecular Biology: A Compendium of Methods from Current Protocols in Molecular Biology, 4th ed., Wiley & Sons, 1999; Harlow and Lane, Antibodies: A Laboratory Manual, Cold Spring Harbor Laboratory Press, 1990; and Harlow and Lane, Using Antibodies: A Laboratory Manual, Cold Spring Harbor Laboratory Press, 1999, Gene Expression Technology (Methods in Enzymology, Vol. 185, edited by D. Goeddel, 1991. Academic Press, San Diego, Calif.), “Guide to Protein Purification” in Methods in Enzymology (M. P. Deutshcer, ed., (1990) Academic Press, Inc.); PCR Protocols: A Guide to Methods and Applications (Innis, et al. 1990. Academic Press, San Diego, Calif.), Culture of Animal Cells: A Manual of Basic Technique, 2^{nd} Ed. (R. I. Freshney. 1987. Liss, Inc. New York, N.Y.), Gene Transfer and Expression Protocols, pp. 109-128, ed. E. J. Murray, The Humana Press Inc., Clifton, N.J.), and the Ambion 1998 Catalog (Ambion, Austin, Tex.), the disclosures of which are incorporated in their entirety herein by reference.

Although methods and materials similar or equivalent to those described herein can be used to practice or test the disclosed technology, suitable methods and materials are described below. The materials, methods, and examples are illustrative only and not intended to be limiting.

As used herein, the terms “immunogenic protein, polypeptide, or peptide” or “antigen” refer to polypeptides or other molecules (or combinations of polypeptides and other molecules) that are immunologically active in the sense that once administered to the host, it is able to evoke an immune response of the humoral and/or cellular type directed against the protein. In embodiments, the protein fragment has substantially the same immunological activity as the total protein. Thus, a protein fragment according to the disclosure can comprise or consist essentially of or consist of at least one epitope or antigenic determinant. An “immunogenic” protein or polypeptide, as used herein, may include the full-length sequence of the protein, analogs thereof, or immunogenic fragments thereof. “Immunogenic fragment” refers to a fragment of a protein which includes one or more epitopes and thus elicits the immunological response described above.

Synthetic antigens are also included within the definition, for example, poly-epitopes, flanking epitopes, and other recombinant or synthetically derived antigens. Immunogenic fragments for purposes of the disclosure may feature at least about 1 amino acid, at least about 3 amino acids, at least about 5 amino acids, at least about 10-15 amino acids, or about 15-25 amino acids or more amino acids, of the molecule. There is no critical upper limit to the length of the fragment, which could comprise nearly the full-length of the protein sequence, or the full-length of the protein sequence, or even a fusion protein comprising at least one epitope of the protein.

As used herein, the term “epitope” refers to the site on an antigen or hapten to which specific B cells and/or T cells respond. The term is also used interchangeably with “antigenic determinant” or “antigenic determinant site”. Antibodies that recognize the same epitope can be identified in a simple immunoassay showing the ability of one antibody to block the binding of another antibody to a target antigen.

As used herein, the term “immunological response” to a composition or vaccine refers to the development in the host of a cellular and/or antibody-mediated immune response to a composition or vaccine of interest. Usually, an “immunological response” includes but is not limited to one or more of the following effects: the production of antibodies, B cells, helper T cells, and/or cytotoxic T cells, directed specifically to an antigen or antigens included in the composition or vaccine of interest. The host may display either a therapeutic or protective immunological response so resistance to new infection will be enhanced and/or the clinical severity of the disease reduced. Such protection will be demonstrated by either a reduction or lack of symptoms normally displayed by an infected host, a quicker recovery time and/or a lowered viral titer in the infected host.

As used herein, the term “variant” refers to a substantially similar sequence. For polynucleotides, a variant comprises a deletion and/or addition and/or change of one or more nucleotides at one or more sites within the native polynucleotide and/or a substitution of one or more nucleotides at one or more sites in the native polynucleotide. As used herein, a “native” polynucleotide or polypeptide comprises a naturally occurring nucleotide sequence or an amino acid sequence, respectively. Variants of a particular polynucleotide of the disclosure (e.g., the reference polynucleotide) can also be evaluated by comparison of the percent sequence identity between the polypeptide encoded by a variant polynucleotide and the polypeptide encoded by the reference polynucleotide. “Variant” protein is intended to mean a protein derived from the native protein by deletion or addition of one or more amino acids at one or more sites in the native protein and/or substitution of one or more amino acids at one or more sites in the native protein. Variant proteins encompassed by the present disclosure are biologically active, that is they have the ability to elicit an immune response.

The HLA-DR/HLA-A*0201/hACE2 triple transgenic mouse model referred to herein is a novel susceptible animal model for pre-clinical testing of human COVID-19 vaccine candidates derived from crossing ACE2 transgenic mice with the unique HLA-DR/HLA-A*0201 double transgenic mice. ACE2 transgenic mice are a hACE2 transgenic mouse model expressing human ACE2 receptors in the lung, heart, kidney and intestine (Jackson Laboratory, Bar Harbor, Me.). The HLA-DR/HLA-A*0201 double transgenic mice are “humanized” HLA double transgenic mice expressing Human Leukocyte Antigen HLA-A*0201 class I and HLA DR*0101 class II in place of the corresponding mouse MHC molecules (which are knocked out). The HLA-A*0201 haplotype was chosen because it is highly represented (>50%) in the human population, regardless of race or ethnicity. The HLA-DR/HLA-A*0201/hACE2 triple transgenic mouse model is a “humanized” transgenic mouse model and has three advantages: (1) it is susceptible to human SARS-CoV2 infection; (2) it develops symptoms similar to those seen in COVID-19 in humans; and (3) it develops CD4⁺ T cells and CD8⁺ T cells response to human epitopes. The novel HLA-DR/HLA-A*0201/hACE2 triple transgenic mouse model of the present invention may be used in the pre-clinical testing of safety, immunogenicity and protective efficacy of the human multi-epitope COVID-19 vaccine candidates of the present invention.

As used herein, the terms “treat” or “treatment” or “treating” refers to both therapeutic treatment and prophylactic or preventative measures, wherein the object is to prevent or slow the development of the disease, such as slow down the development of a disorder, or reducing at least one adverse effect or symptom of a condition, disease or disorder, e.g., any disorder characterized by insufficient or undesired organ or tissue function. Treatment is generally “effective” if one or more symptoms or clinical markers are reduced as that term is defined herein. Alternatively, a treatment is “effective” if the progression of a disease is reduced or halted. That is, “treatment” includes not just the improvement of symptoms or decrease of markers of the disease, but also a cessation or slowing of progress or worsening of a symptom that would be expected in absence of treatment. Beneficial or desired clinical results include, but are not limited to, alleviation of one or more symptom(s), diminishment of extent of disease, stabilized (e.g., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. “Treatment” also includes ameliorating a disease, lessening the severity of its complications, preventing it from manifesting, preventing it from recurring, merely preventing it from worsening, mitigating an inflammatory response included therein, or a therapeutic effort to affect any of the aforementioned, even if such therapeutic effort is ultimately unsuccessful.

As used herein, the term “carrier” or “pharmaceutically acceptable carrier” or “pharmaceutically acceptable vehicle” refers to any appropriate or useful carrier or vehicle for introducing a composition to a subject. Pharmaceutically acceptable carriers or vehicles may be conventional but are not limited to conventional vehicles. For example, E. W. Martin, Remington's Pharmaceutical Sciences, Mack Publishing Co., Easton, Pa., 15th Edition (1975) and D. B. Troy, ed. Remington: The Science and Practice of Pharmacy, Lippincott Williams & Wilkins, Baltimore Md. and Philadelphia, Pa., 21^{st} Edition (2006) describe compositions and formulations suitable for pharmaceutical delivery of one or more therapeutic compounds or molecules. Carriers (e.g., pharmaceutical carriers, pharmaceutical vehicles, pharmaceutical compositions, pharmaceutical molecules, etc.) are materials generally known to deliver molecules, proteins, cells and/or drugs and/or other appropriate material into the body. In general, the nature of the carrier will depend on the nature of the composition being delivered as well as the particular mode of administration being employed. In addition to biologically-neutral carriers, pharmaceutical compositions administered may contain minor amounts of non-toxic auxiliary substances, such as wetting or emulsifying agents, preservatives, and pH buffering agents and the like. Patents that describe pharmaceutical carriers include, but are not limited to: U.S. Pat. Nos. 6,667,371; 6,613,355; 6,596,296; 6,413,536; 5,968,543; 4,079,038; 4,093,709; 4,131,648; 4,138,344; 4,180,646; 4,304,767; 4,946,931, the disclosures of which are incorporated in their entirety by reference herein. The carrier may, for example, be solid, liquid (e.g., a solution), foam, a gel, the like, or a combination thereof. In some embodiments, the carrier comprises a biological matrix (e.g., biological fibers, etc.). In some embodiments, the carrier comprises a synthetic matrix (e.g., synthetic fibers, etc.). In certain embodiments, a portion of the carrier may comprise a biological matrix and a portion may comprise synthetic matrix.

As used herein “coronavirus” may refer to a group of related viruses such as but not limited to severe acute respiratory syndrome (SARS), middle east respiratory syndrome (MERS), and severe acute respiratory syndrome coronavirus 2 (SARS-CoV-2). All the coronaviruses cause respiratory tract infection that range from mild to lethal in mammals. Several non-limiting examples of Coronavirus strains are described herein. In some embodiments, the compositions may protect against any Sarbecoviruses including but not limited to SARS-CoV1 or SARS-CoV2. As used herein, “severe acute respiratory syndrome coronavirus 2 (SARS-CoV2)” is a betacoronavirus that causes Coronavirus Disease 19 (COVID-19).

A “subject” is an individual and includes, but is not limited to, a mammal (e.g., a human, horse, pig, rabbit; dog, sheep, goat, non-human primate, cow, cat, guinea pig, or rodent), a fish, a bird, a reptile or an amphibian. The term does not denote a particular age or sex. Thus, adult and newborn subjects, as well as fetuses, whether male or female, are intended to be included. A “patient” is a subject afflicted with a disease or disorder. The term “patient” includes human and veterinary subjects

The terms “administering” and “administration” refer to methods of providing a pharmaceutical preparation to a subject. Such methods are well known to those skilled in the art and include, but are not limited to, administering the compositions orally, parenterally (e.g., intravenously and subcutaneously), by intramuscular injection, by intraperitoneal injection, intrathecally, transdermally, extracorporeally, topically or the like.

A composition can also be administered by topical intranasal administration (intranasally) or administration by inhalant. As used herein, “topical intranasal administration” means delivery of the compositions into the nose and nasal passages through one or both of the nares and can comprise delivery by a spraying mechanism (device) or droplet mechanism (device), or through aerosolization of the composition. Administration of the compositions by inhalant can be through the nose or mouth via delivery by a spraying or droplet mechanism. As used herein, “an inhaler” can be a spraying device or a droplet device for delivering a composition comprising the vaccine composition, in a pharmaceutically acceptable carrier, to the nasal passages and the upper and/or lower respiratory tracts of a subject. Delivery can also be directly to any area of the respiratory system (e.g., lungs) via intratracheal intubation. The exact amount of the compositions required will vary from subject to subject, depending on the species, age, weight and general condition of the subject, the severity of the disorder being treated, the particular composition used, its mode of administration and the like. Thus, it is not possible to specify an exact amount for every composition. However, an appropriate amount can be determined by one of ordinary skill in the art using only routine experimentation given the teachings herein.

A composition can also be administered by buccal delivery or by sublingual delivery. As used herein “buccal delivery” may refer to a method of administration in which the compound is delivered through the mucosal membranes lining the cheeks. In some embodiment, for a buccal delivery the vaccine composition is placed between the gum and the cheek of a patient. As used herein “sublingual delivery” may refer to a method of administration in which the compound is delivered through the mucosal membrane under the tongue. In some embodiments, for a sublingual delivery the vaccine composition is administered under the tongue of a patient.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, for example, U.S. Pat. No. 3,610,795, which is incorporated by reference herein.

### DETAILED DESCRIPTION OF THE INVENTION

Before the present compounds, compositions, and/or methods are disclosed and described, it is to be understood that this invention is not limited to specific synthetic methods or to specific compositions, as such may, of course, vary. It is also to be understood that the terminology used herein is for the purpose of describing particular embodiments only and is not intended to be limiting. Embodiments of the present invention can be freely combined with each other if they are not mutually exclusive.

### Pan-Coronavirus Vaccines

The present invention features preemptive pan-coronavirus vaccines, methods of use, and methods of producing said vaccines, methods of preventing coronavirus infections, etc. The present invention also provides methods of testing said vaccines, e.g., using particular animal models and clinical trials. The vaccine compositions herein can induce efficient and powerful protection against the coronavirus disease or infection, e.g., by inducing the production of antibodies (Abs), CD4+ T helper (Th1) cells, and CD8+ cytotoxic T-cells (CTL).

The vaccine compositions, e.g., the antigens, herein feature multiple large sequences which may comprise multiple conserved epitopes, that helps provide multiple opportunities for the body to develop an immune response for preventing an infection. Further, the vaccines herein may be designed to be effective against past, current, and future coronavirus outbreaks.

The vaccine composition comprises multiple large sequences. In certain embodiments, the large sequences are conserved large sequences, e.g., sequences that are highly conserved among human coronaviruses and/or animal coronaviruses (e.g., coronaviruses isolated from animals susceptible to coronavirus infections).

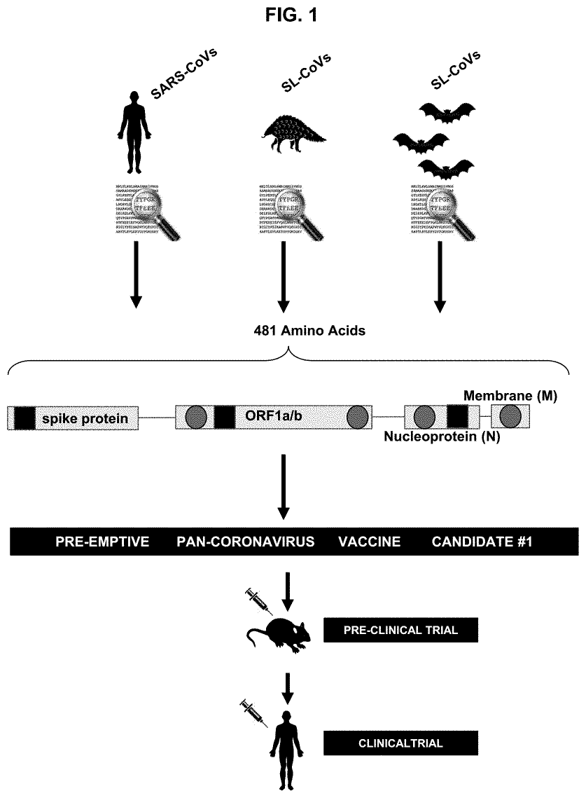

The present invention describes the identification of conserved large sequences comprising B cell, CD4+ T cell, and CD8+ T cell epitopes. For example, FIG. 1 shows a schematic of the development of a pre-emptive pan coronavirus vaccine featuring multiple conserved large sequences comprising multiple B cell epitopes, multiple conserved CD8⁺ T cell epitopes, and multiple CD4+ T cell epitopes. The large sequences are derived from sequence analysis of many coronaviruses.

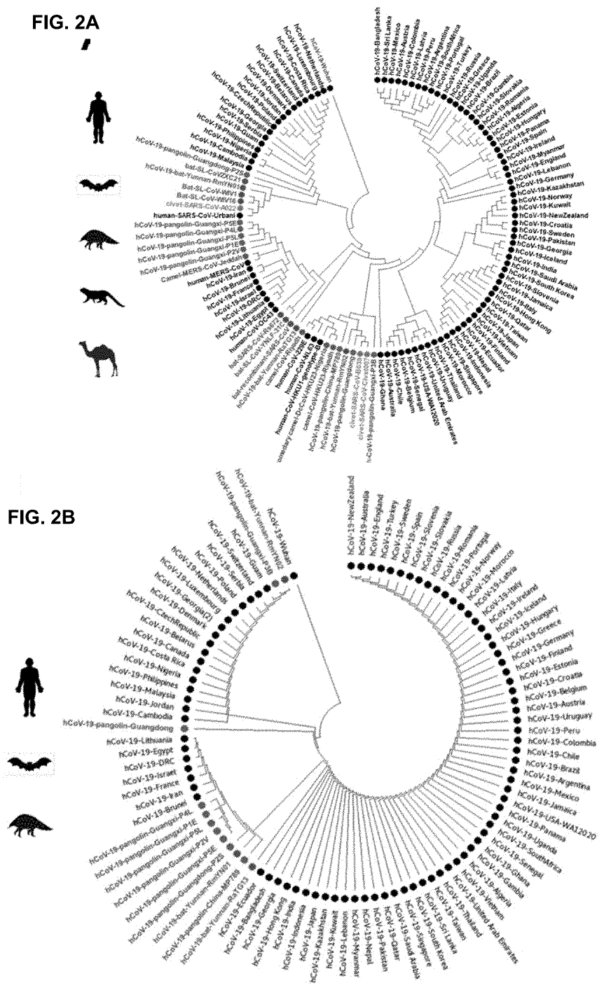

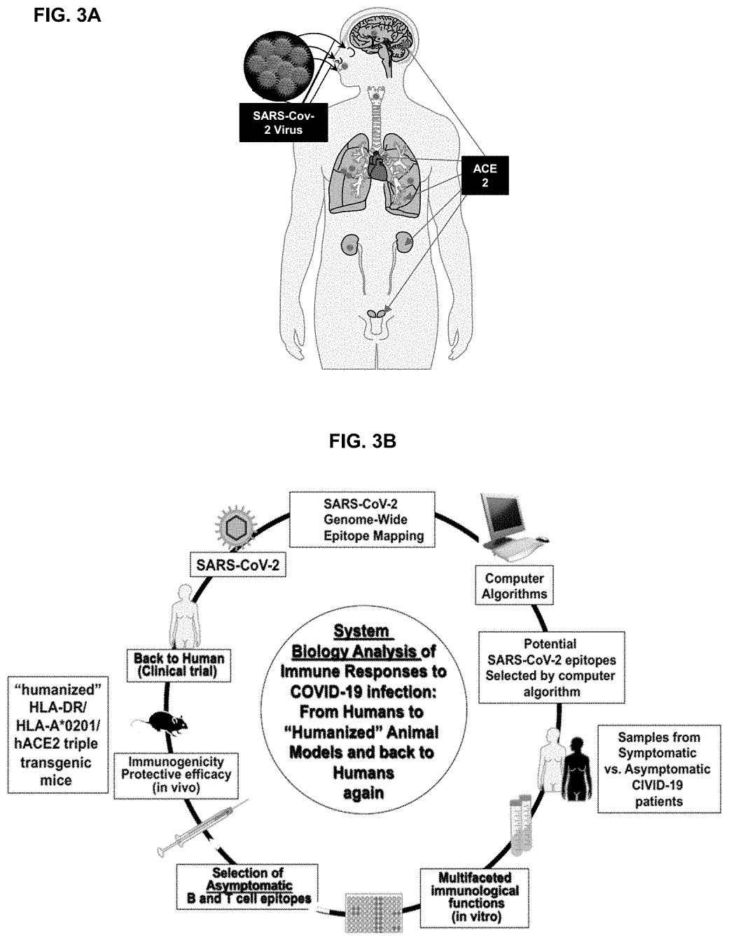

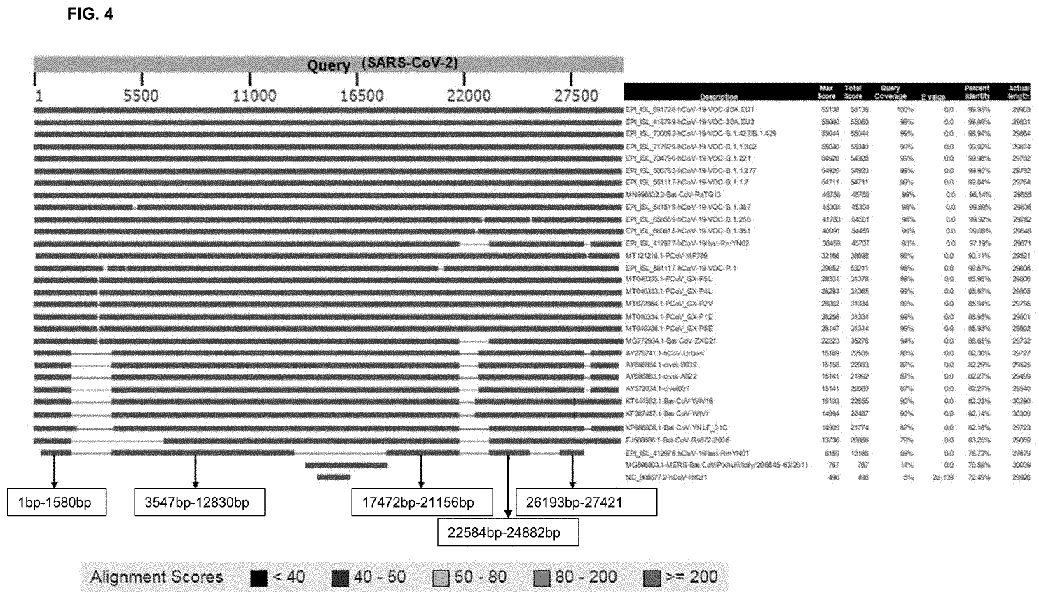

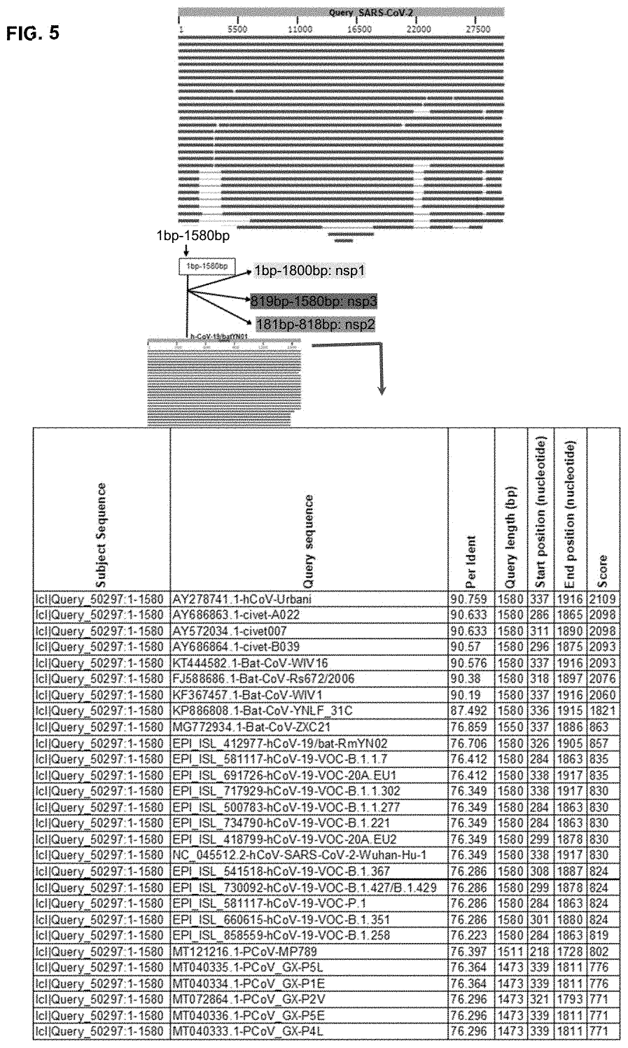

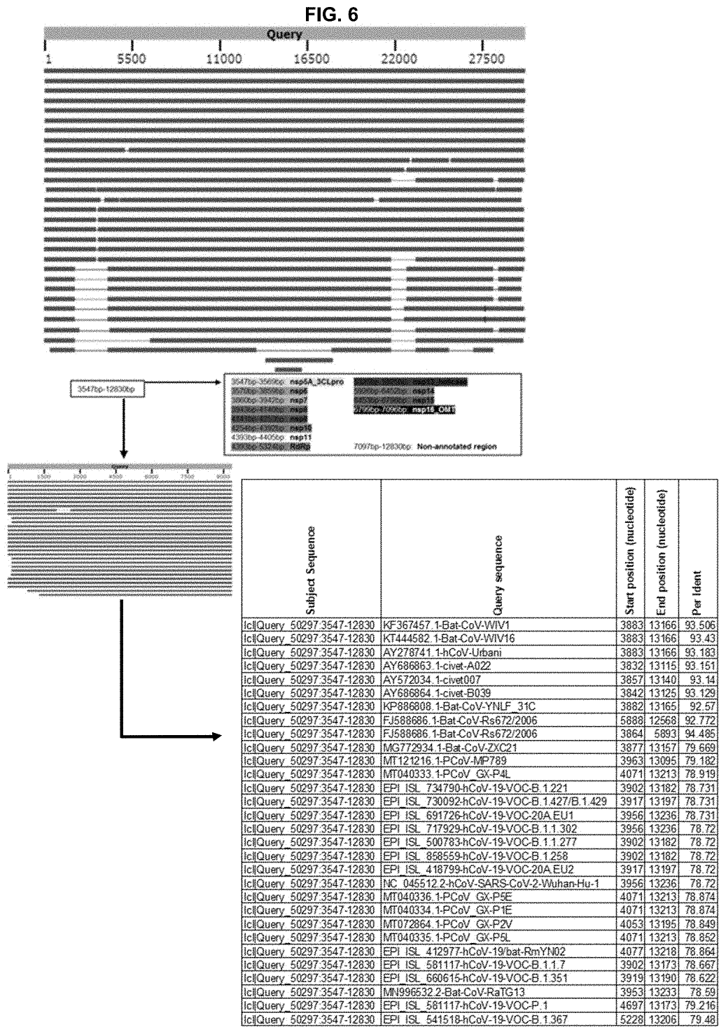

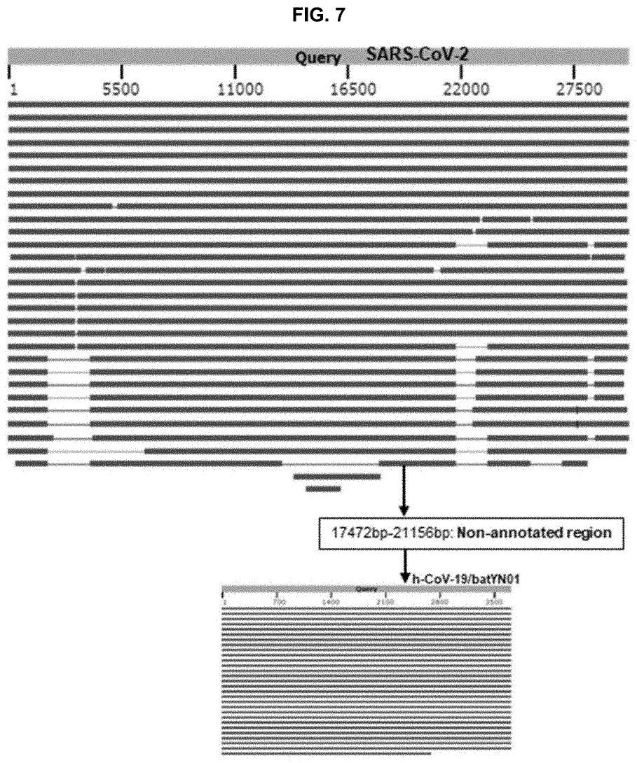

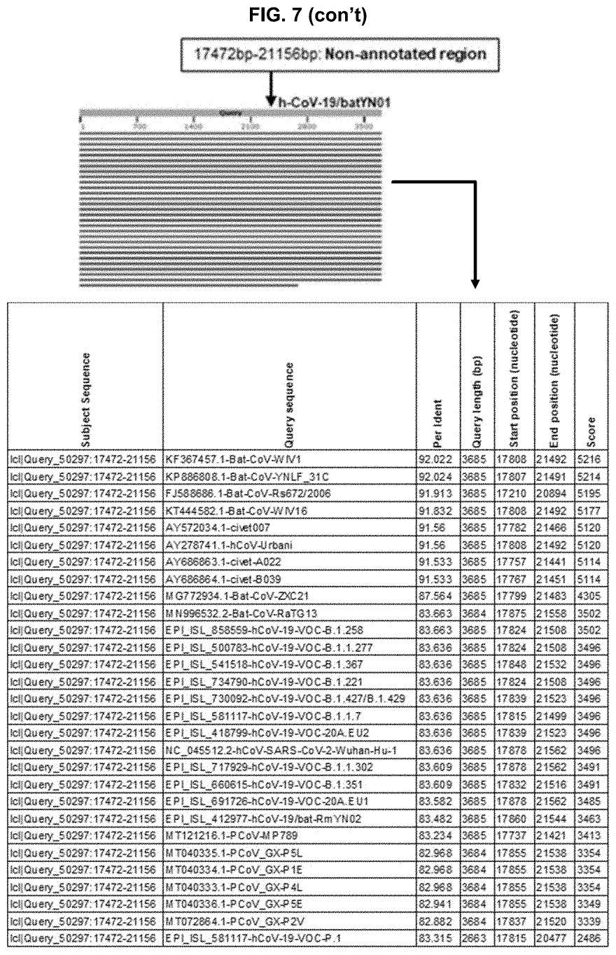

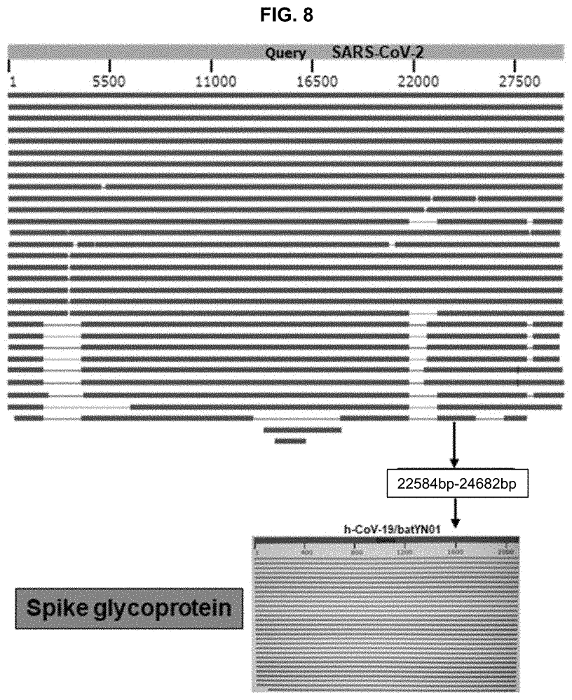

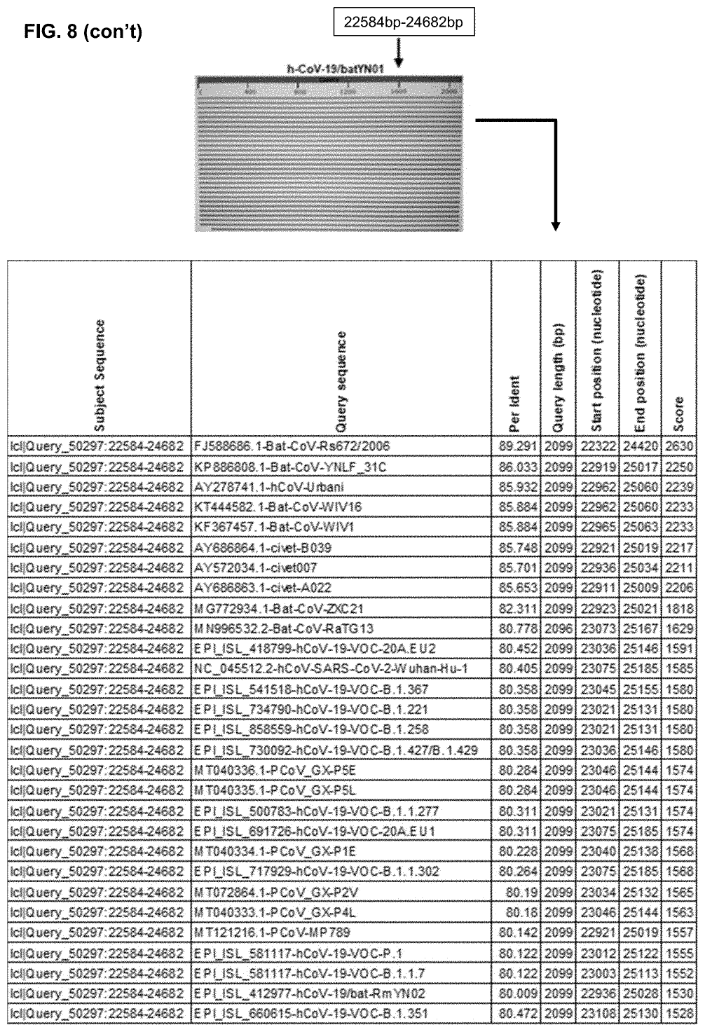

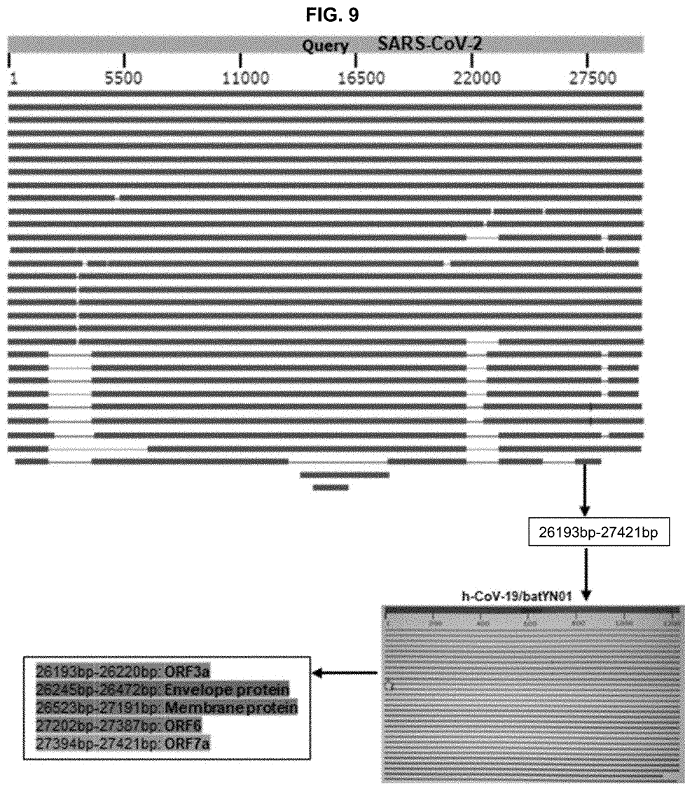

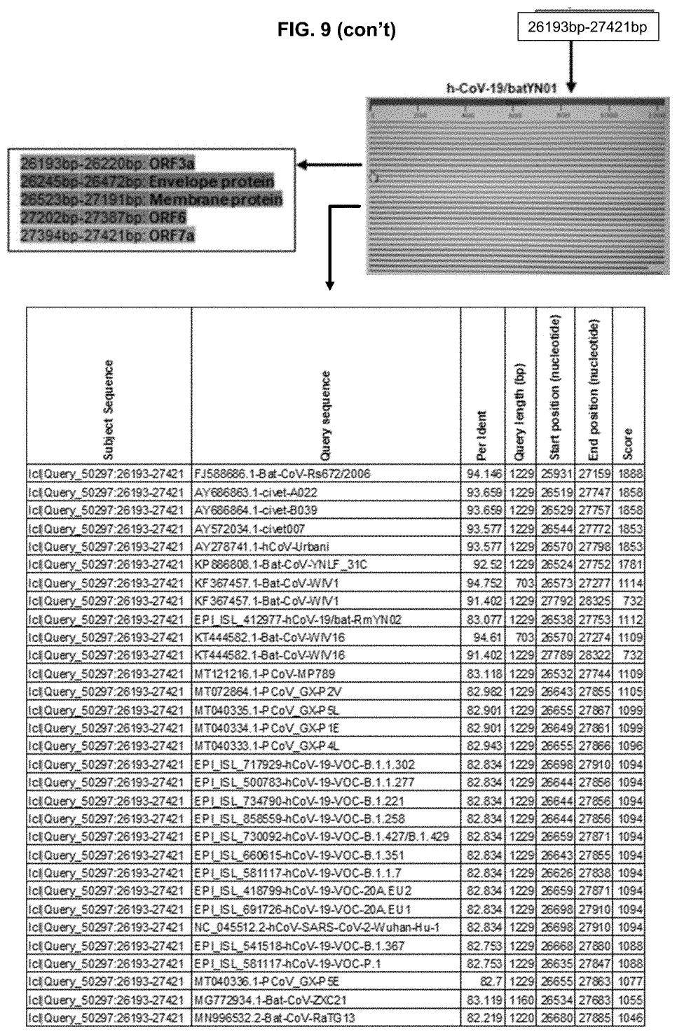

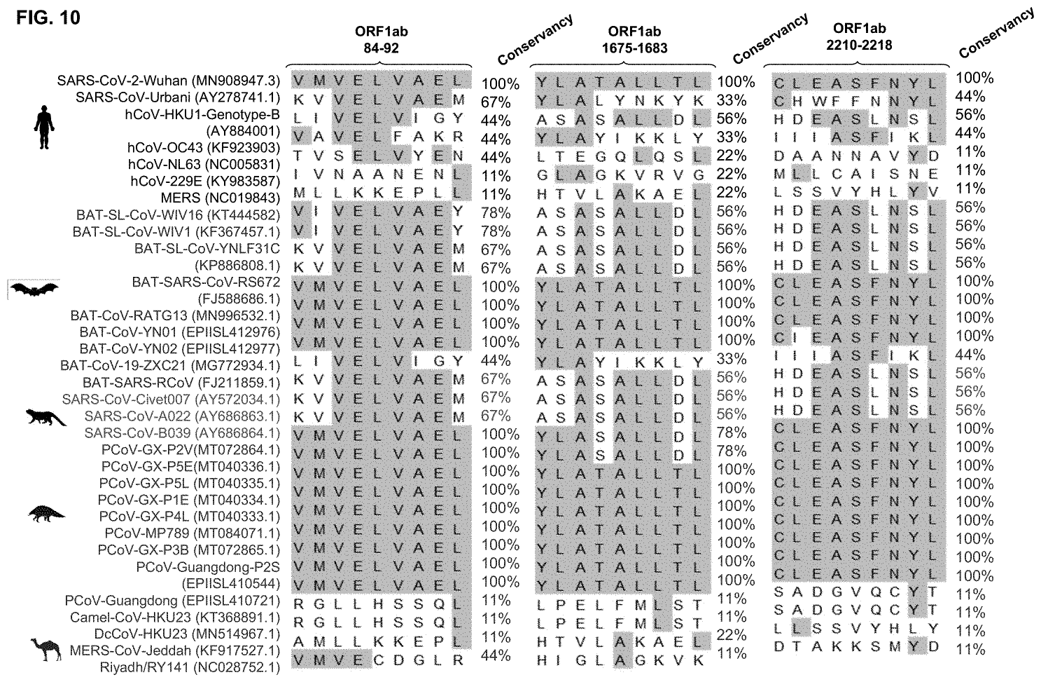

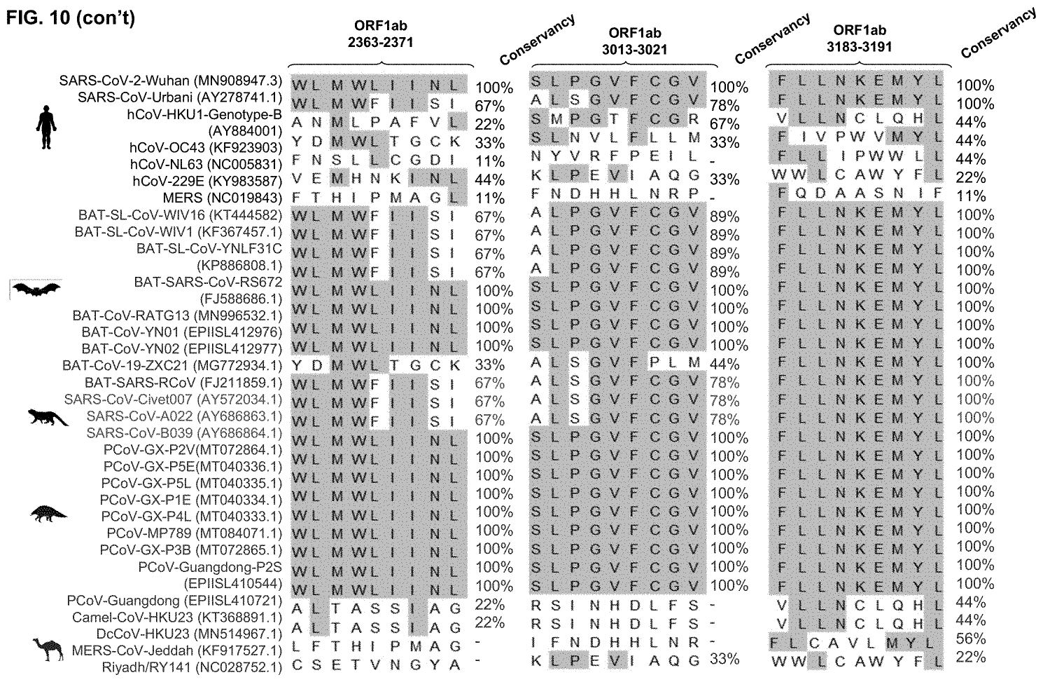

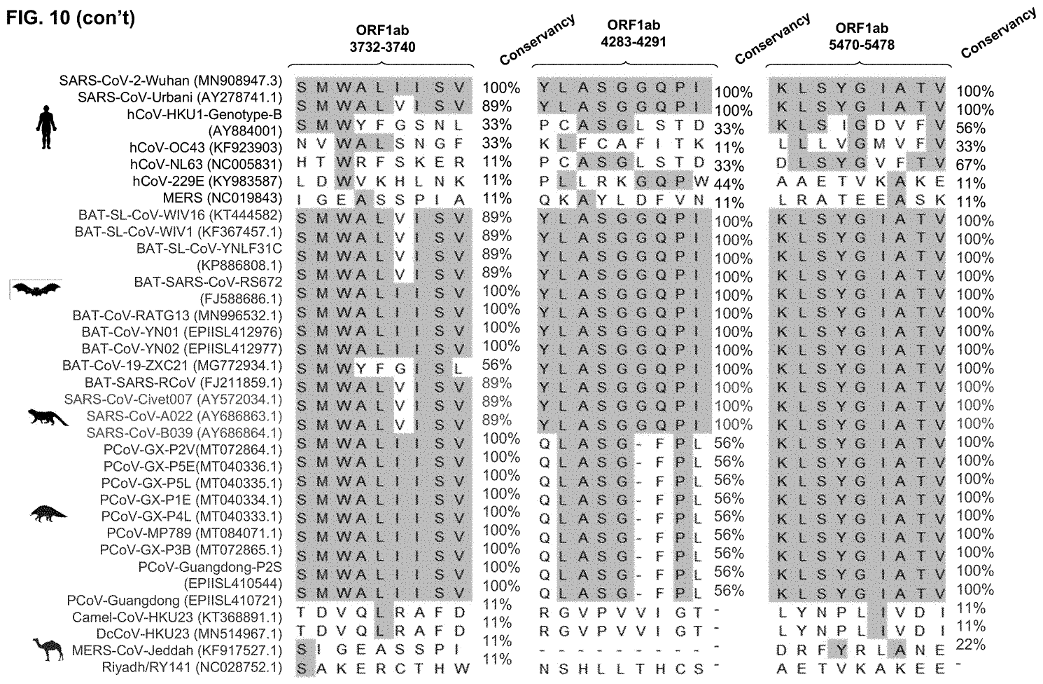

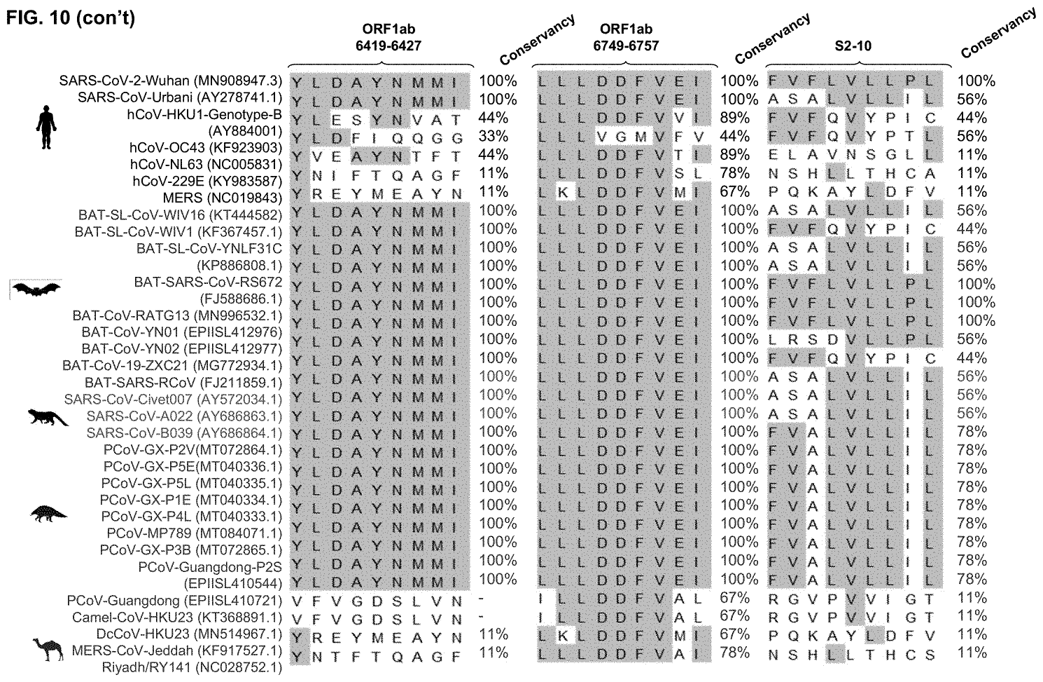

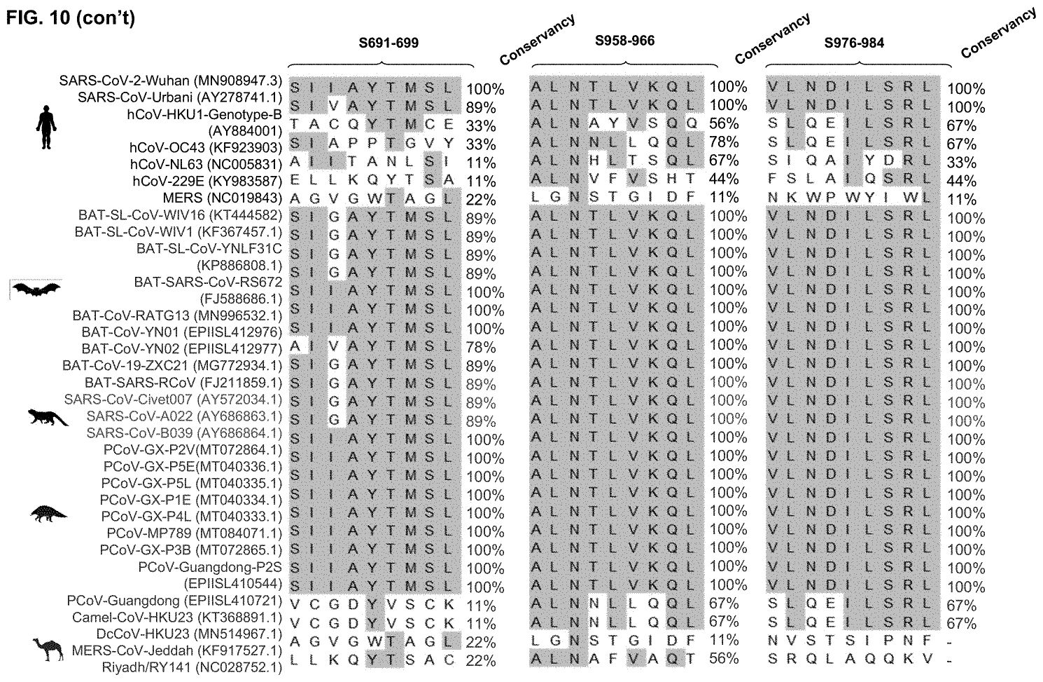

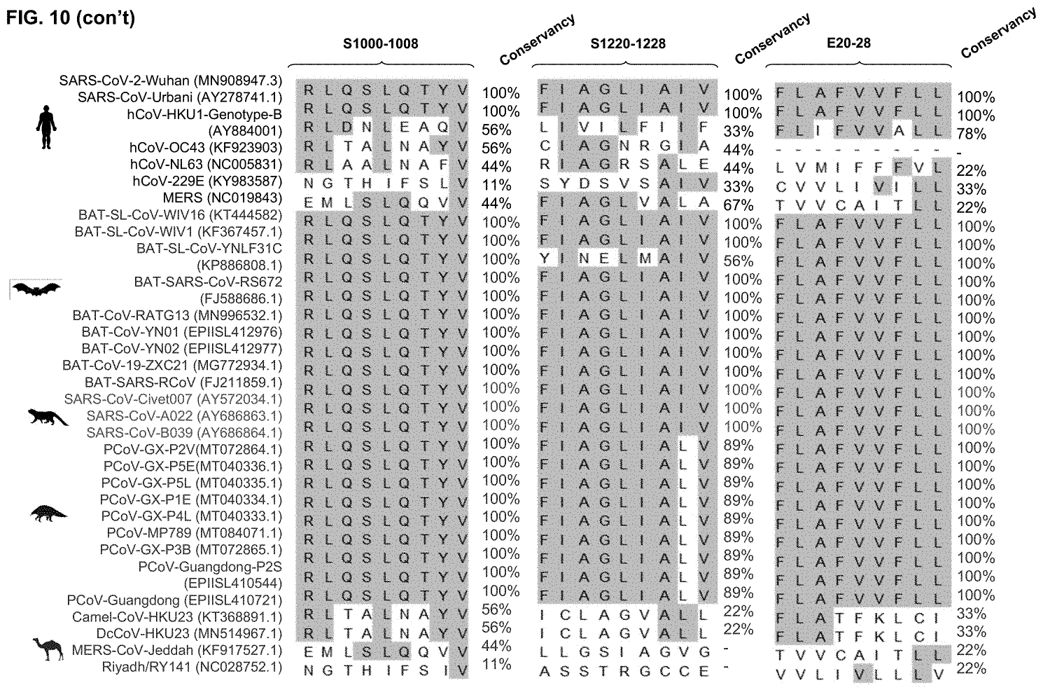

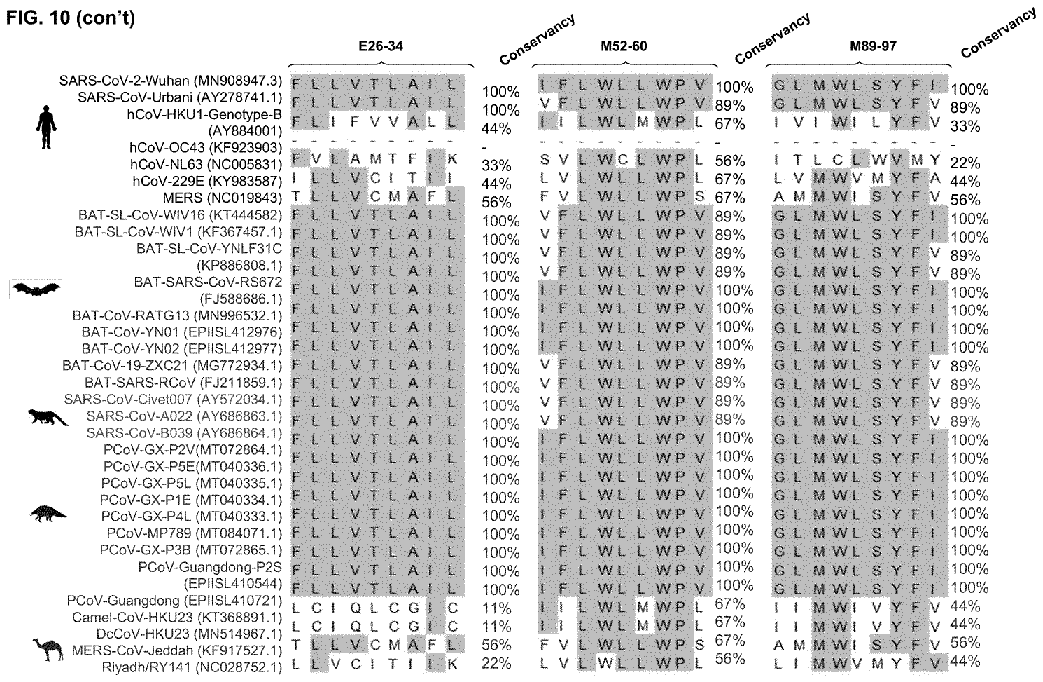

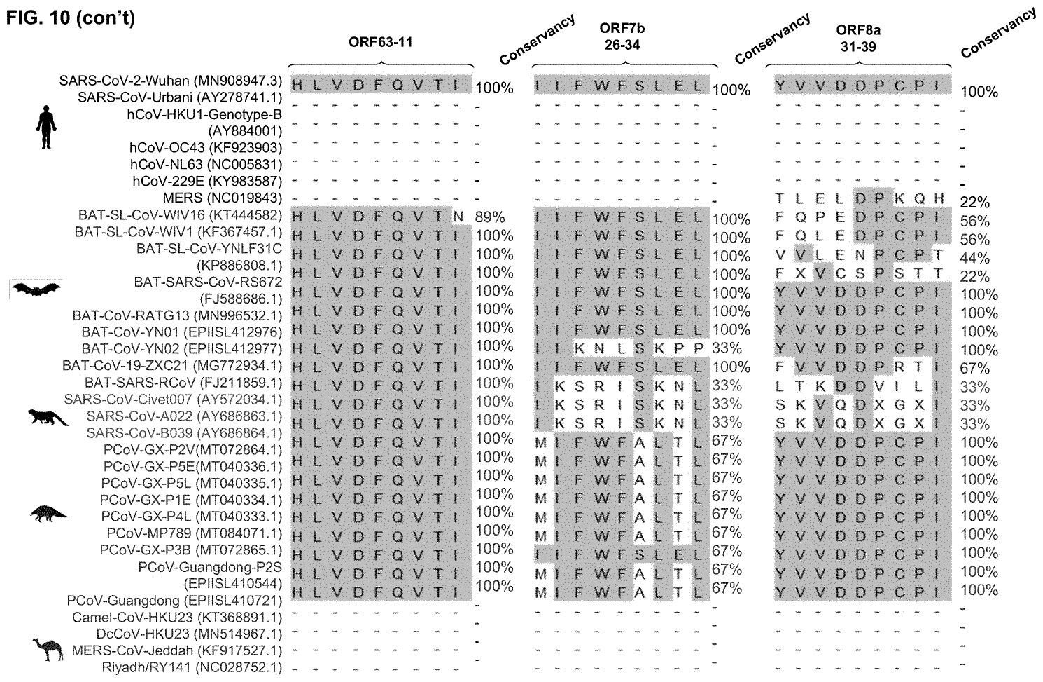

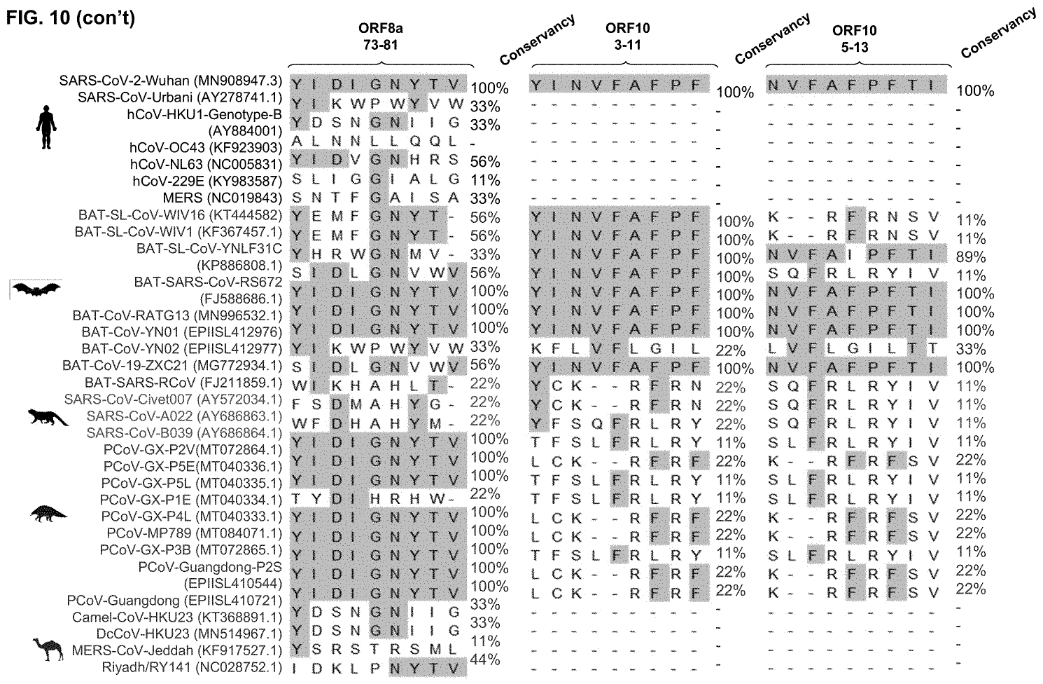

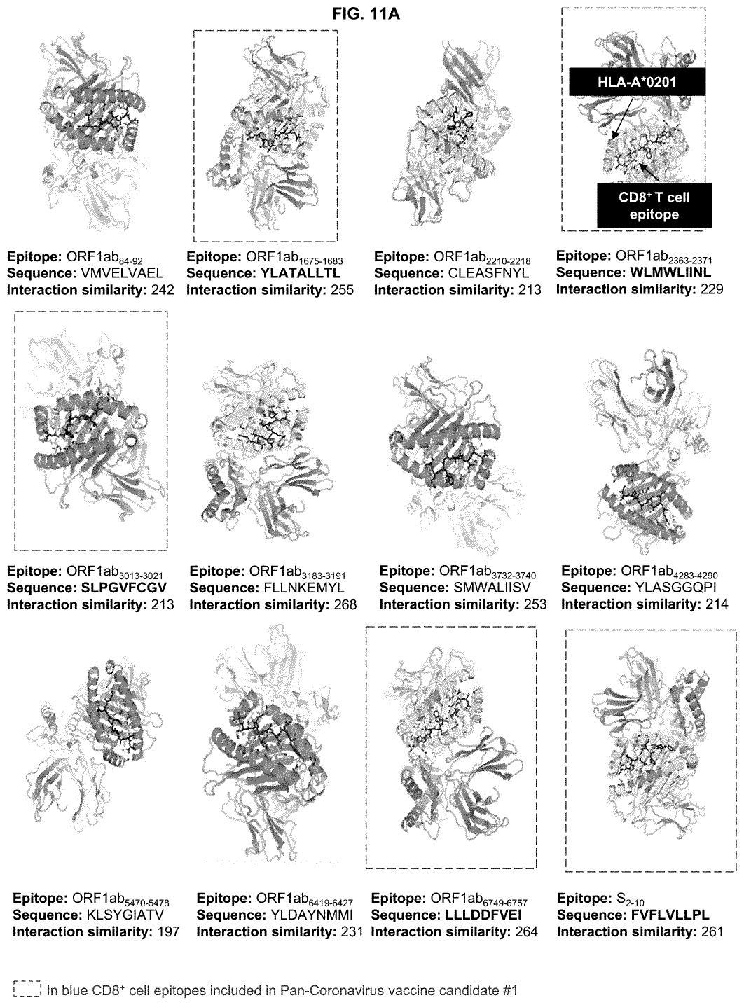

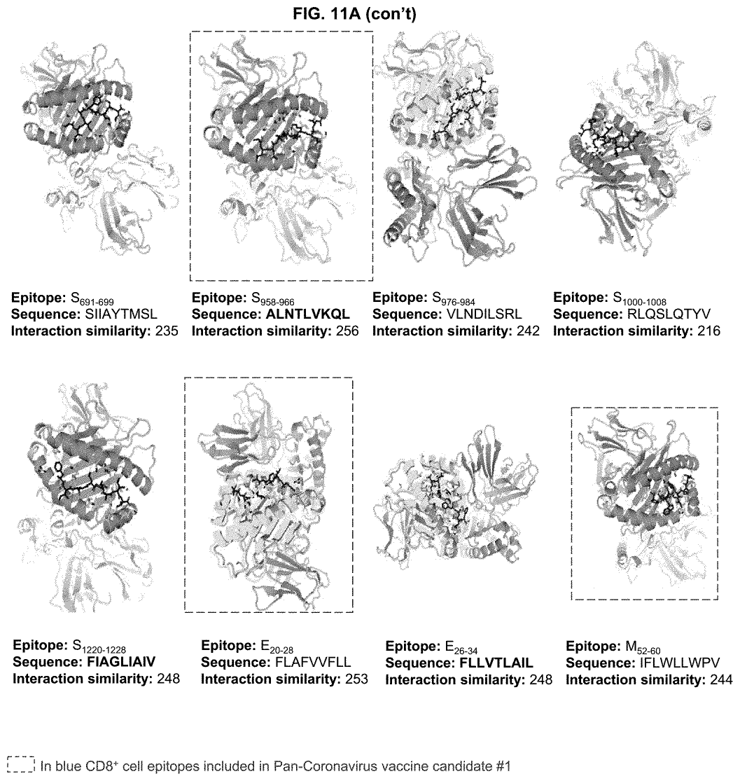

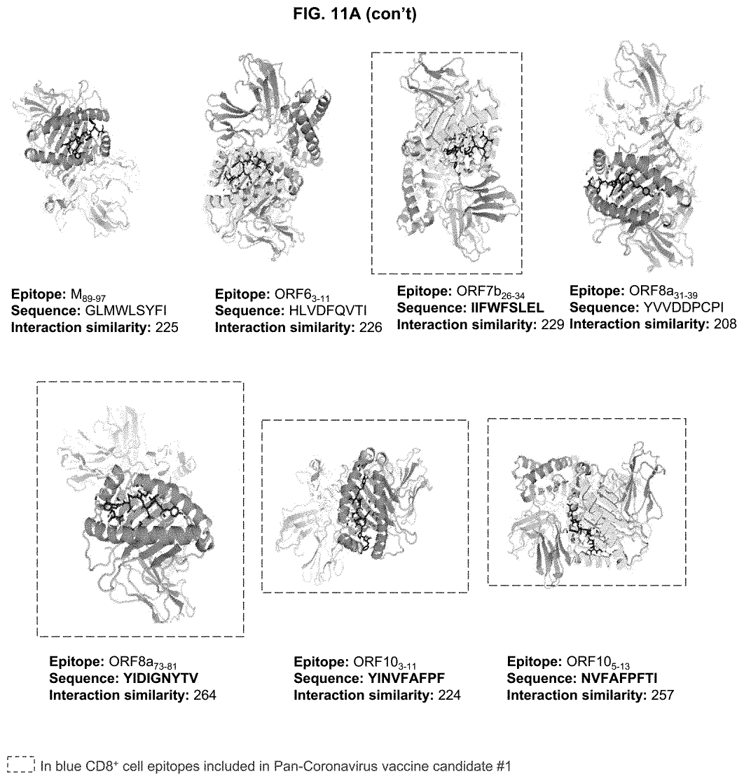

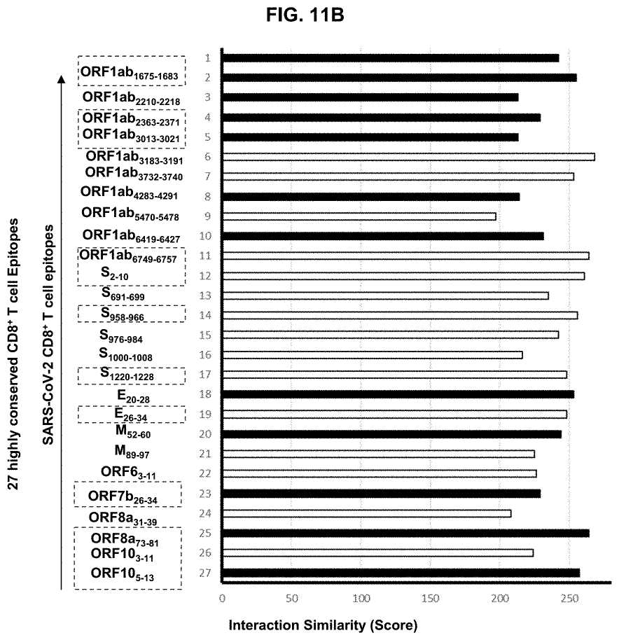

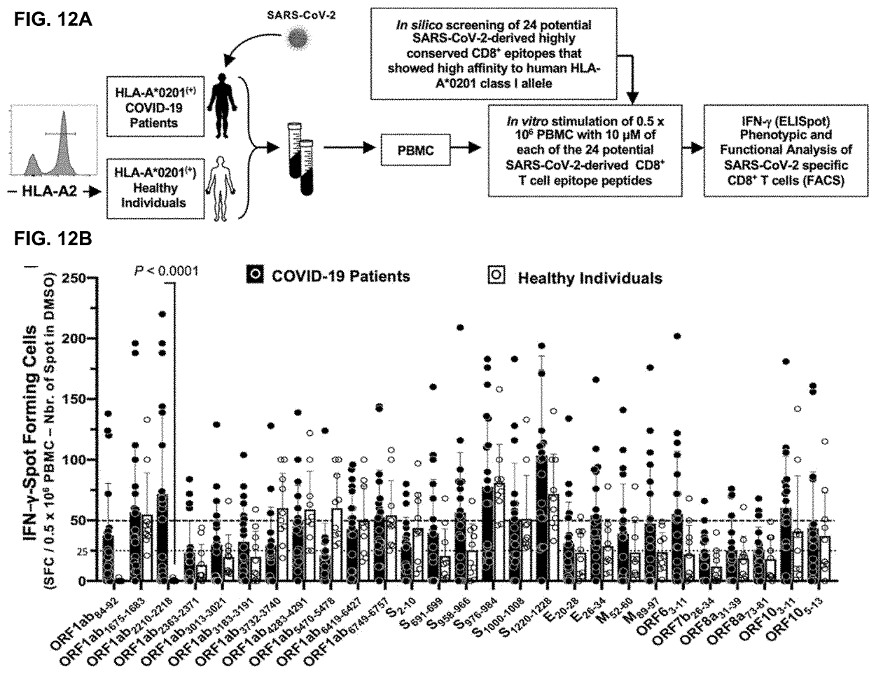

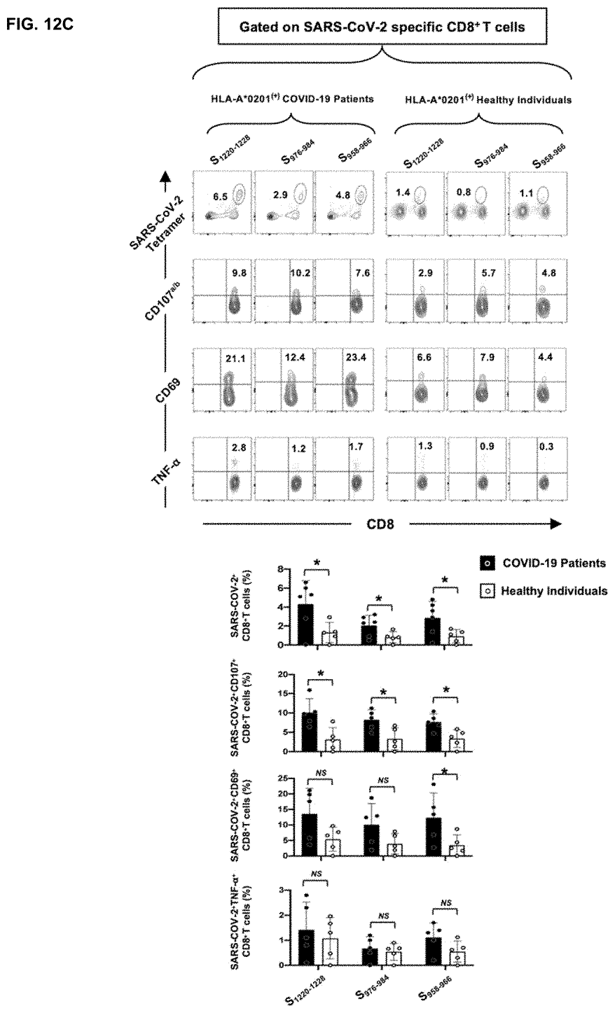

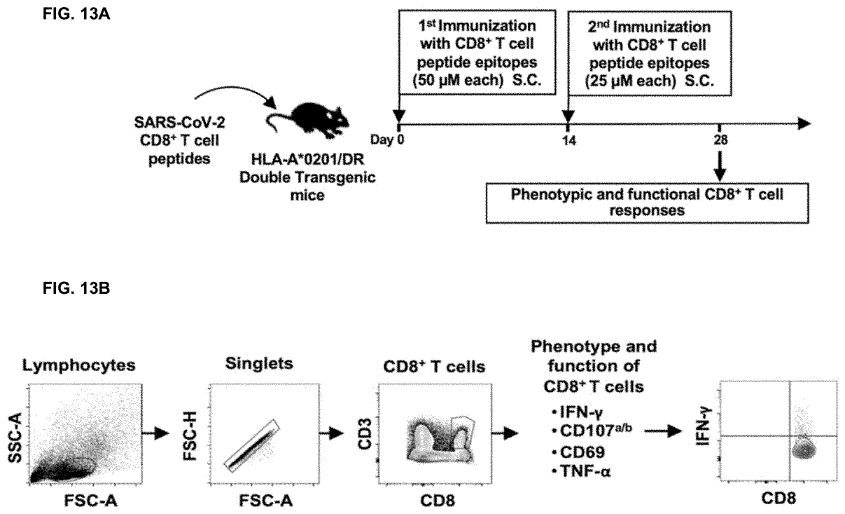

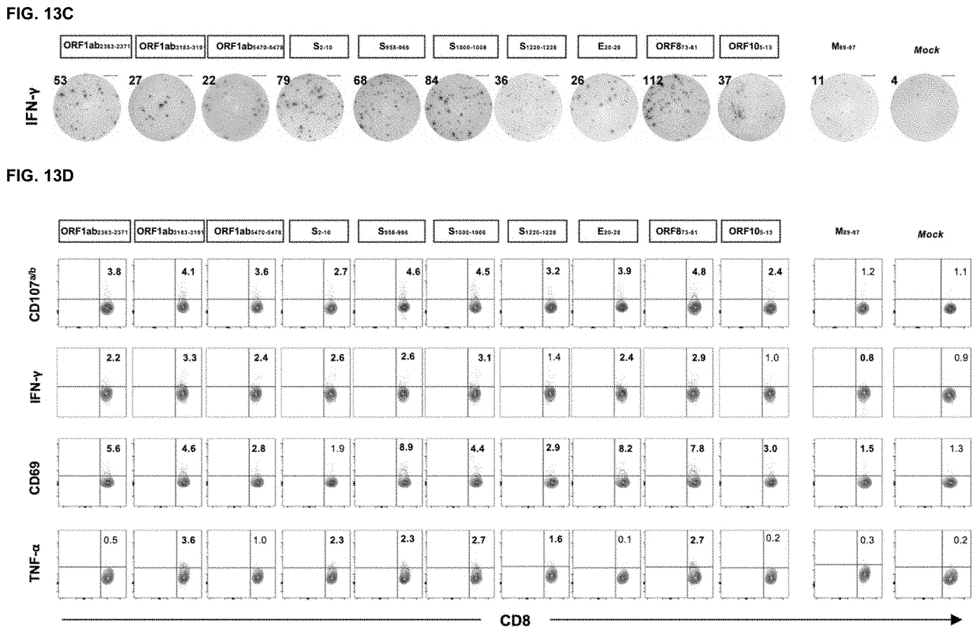

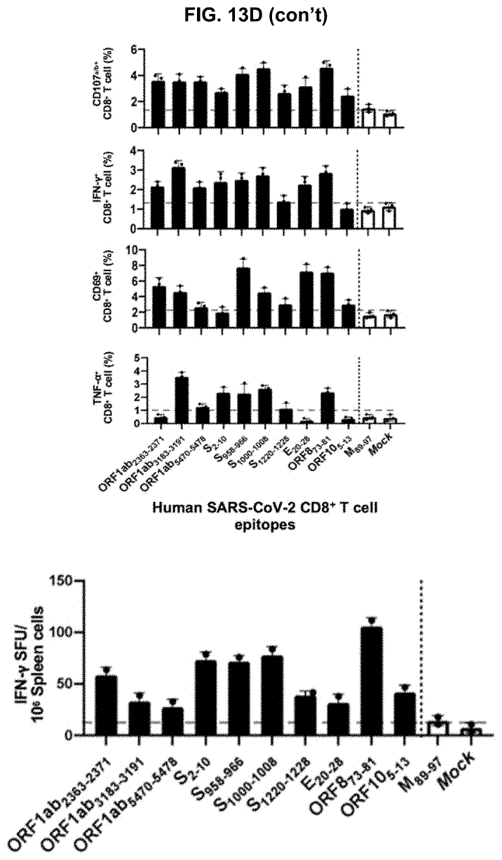

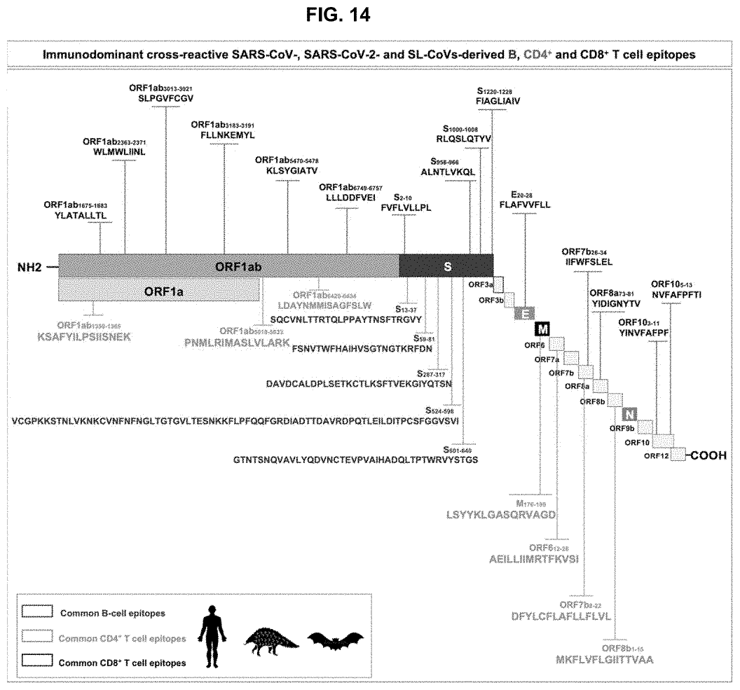

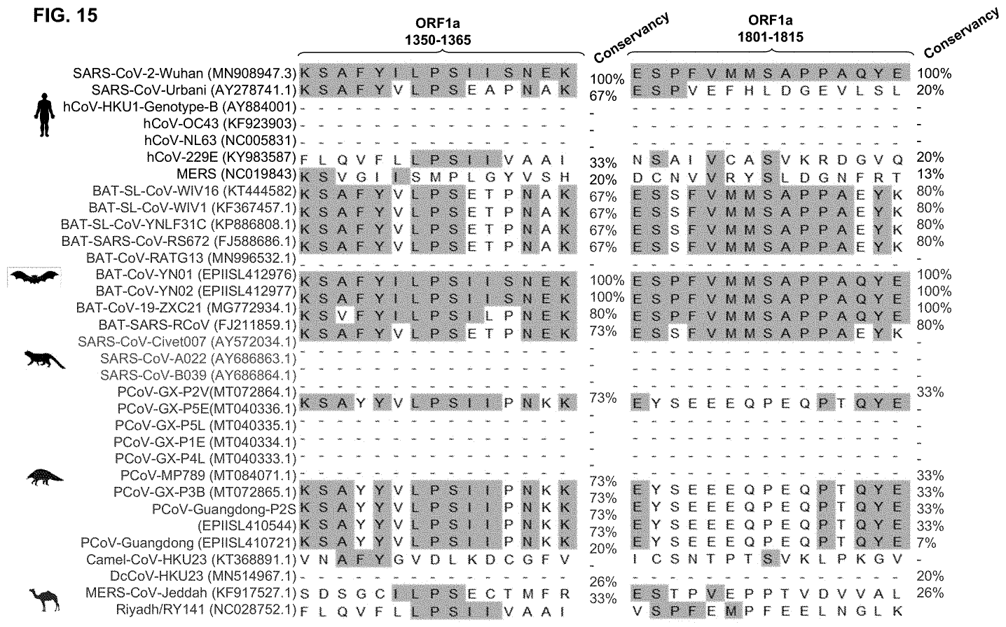

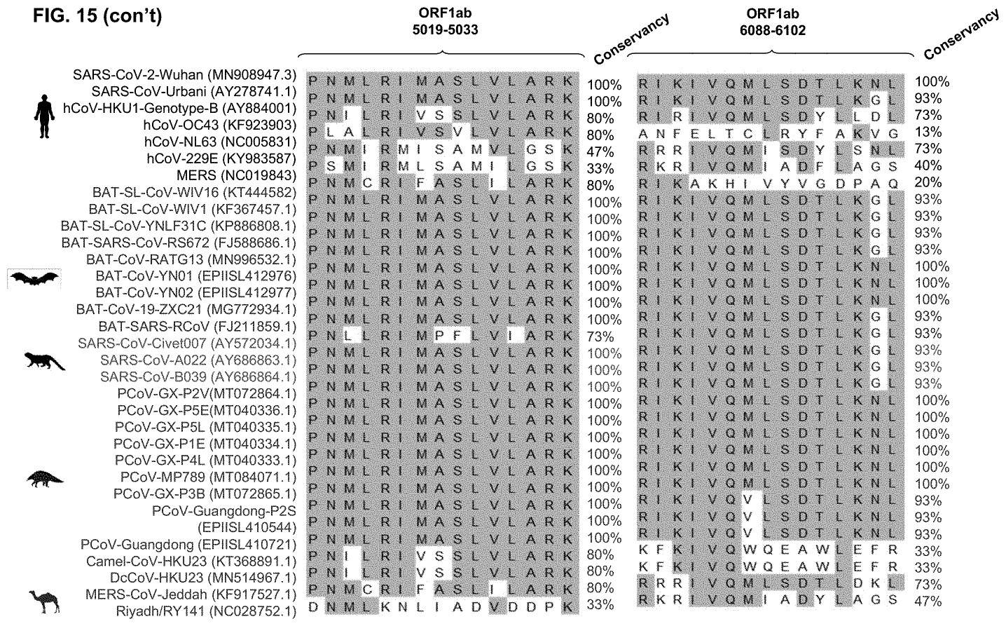

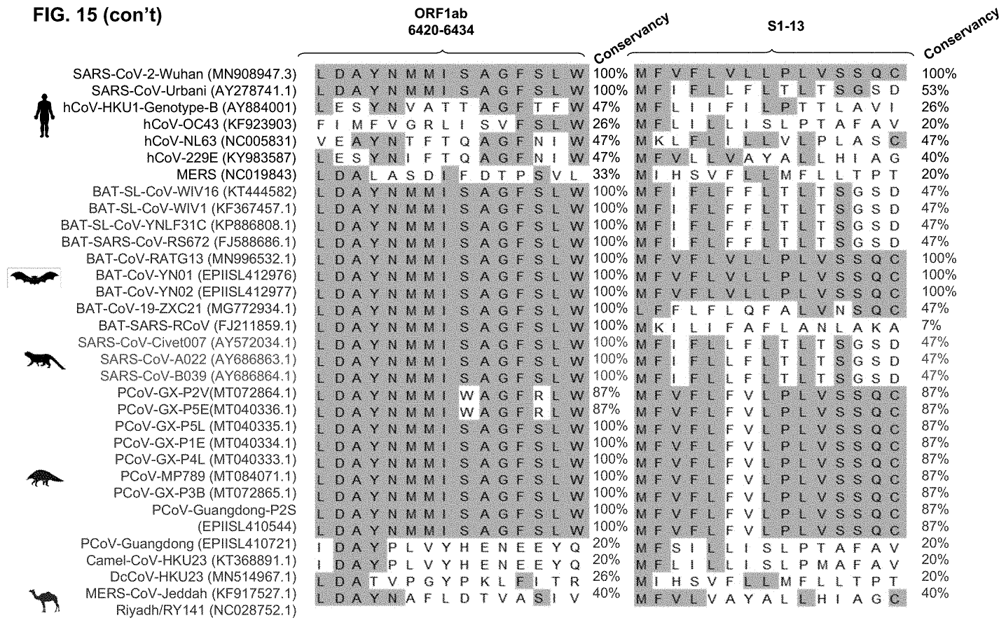

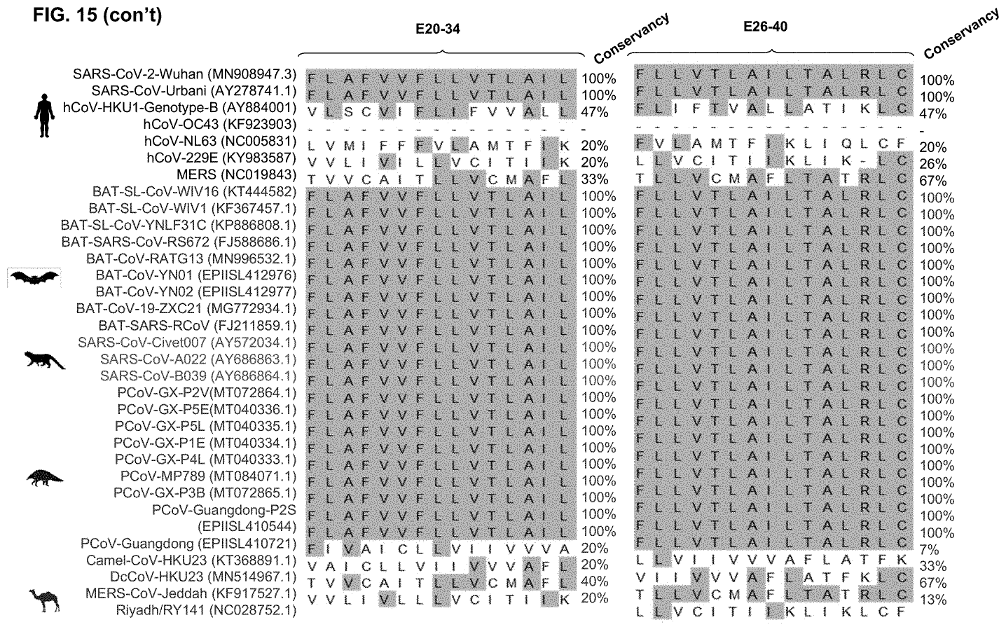

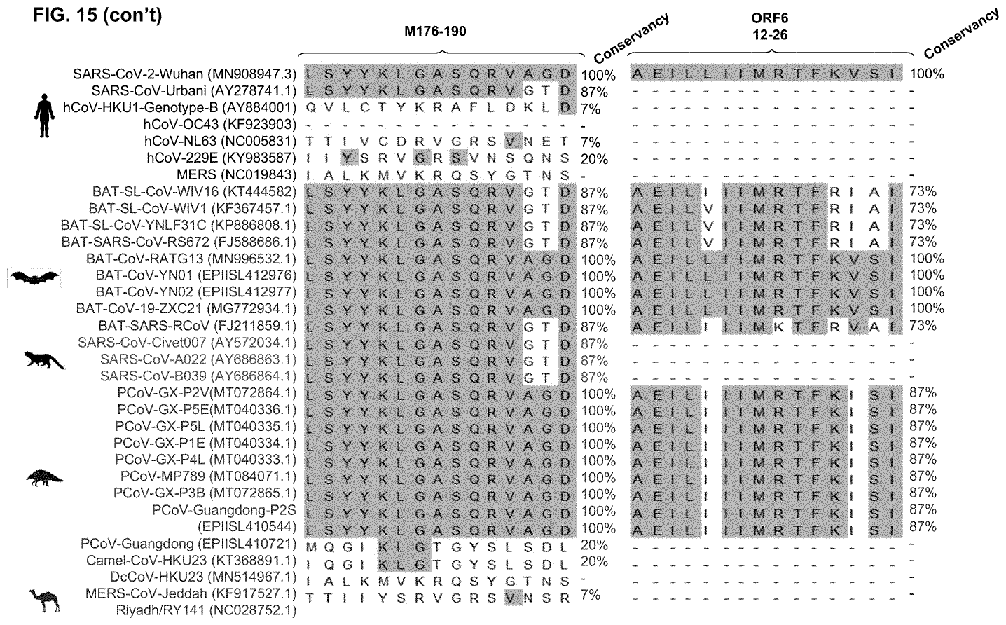

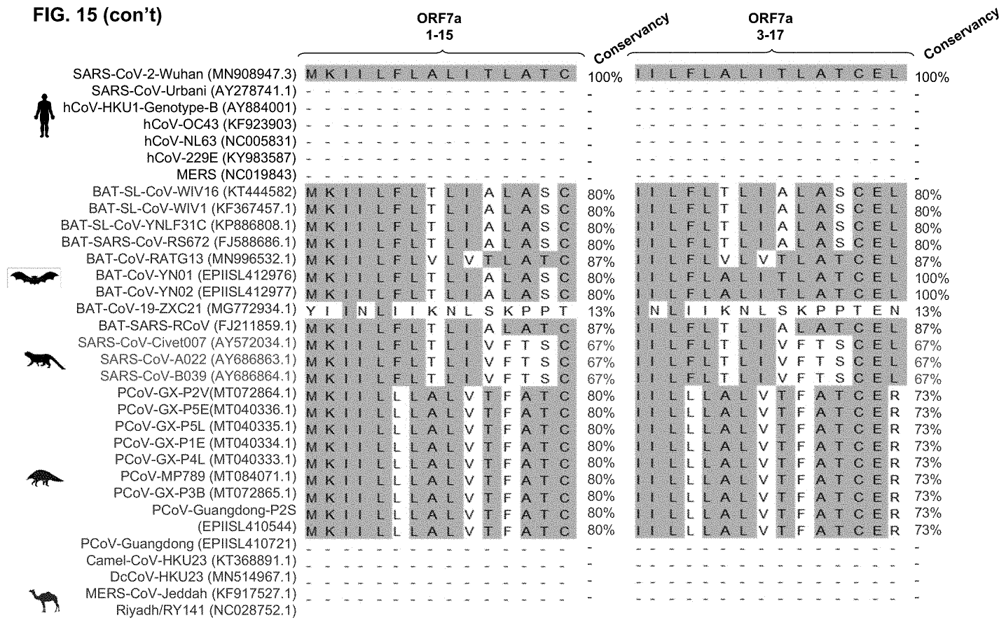

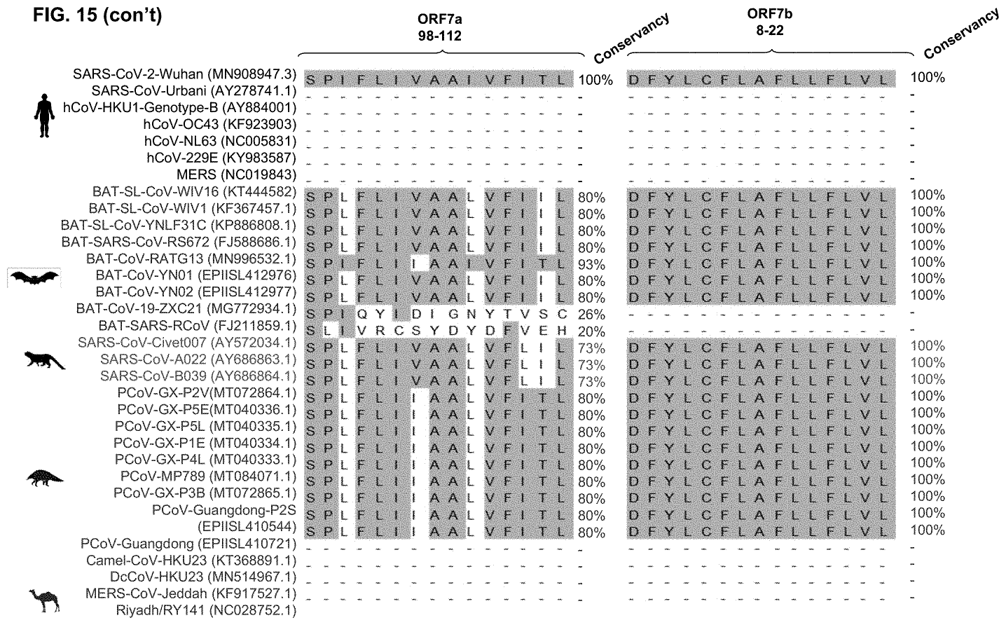

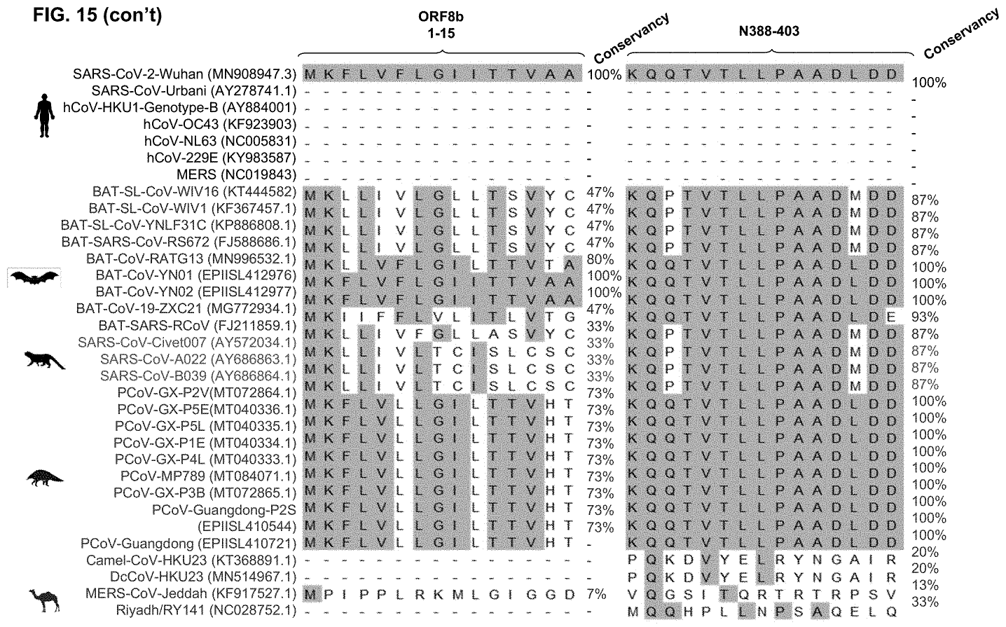

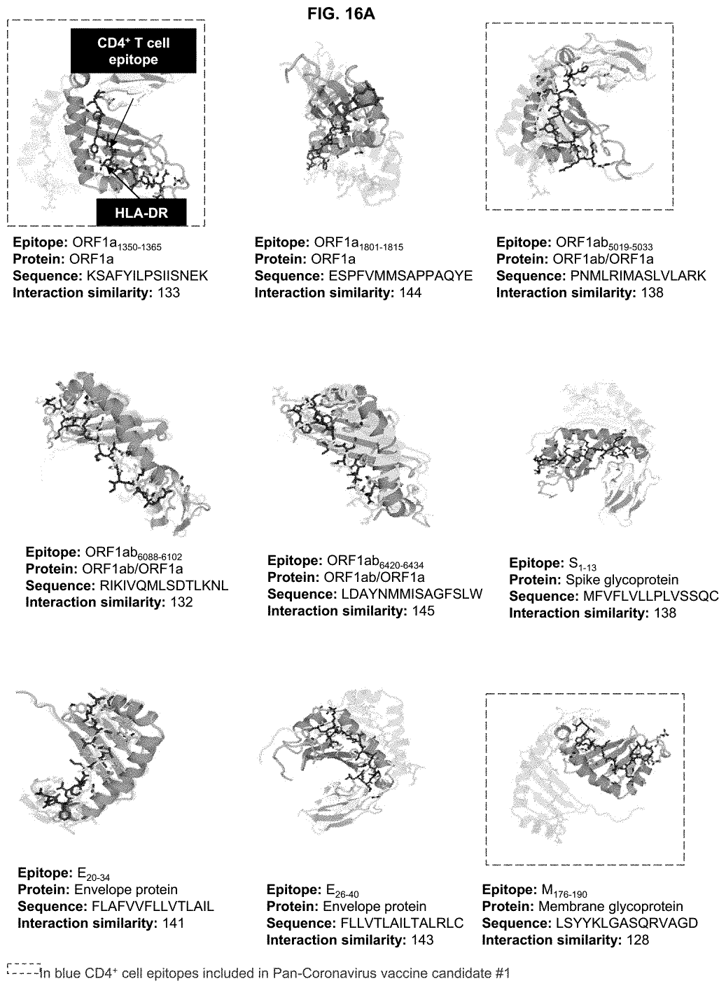

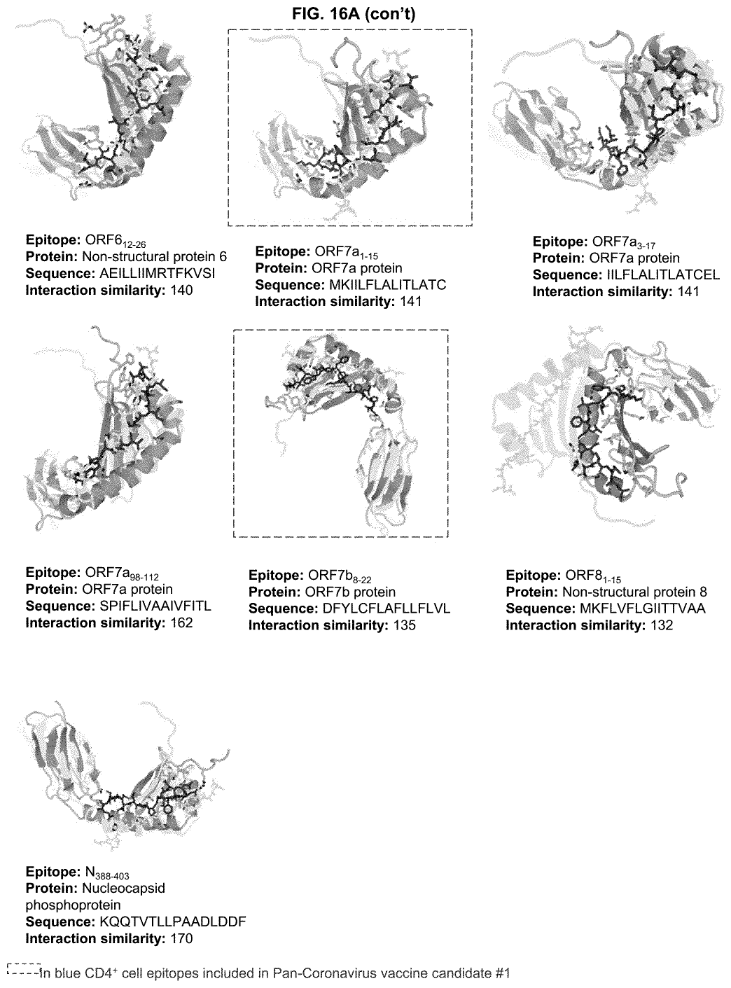

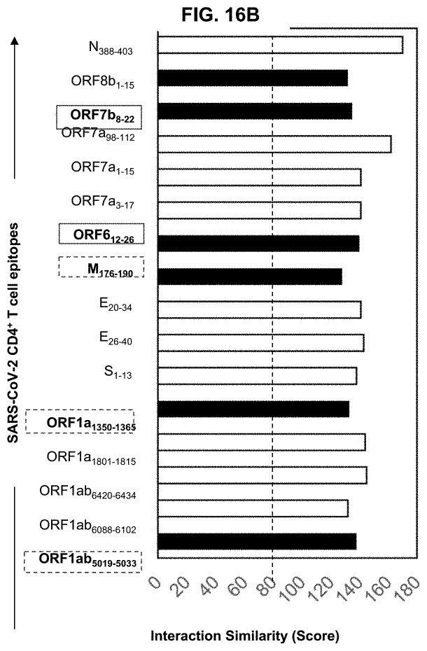

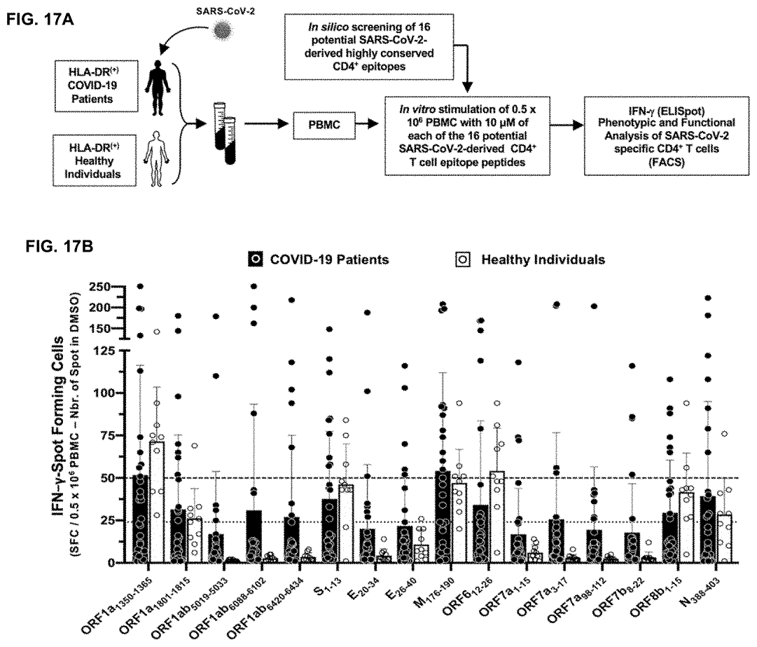

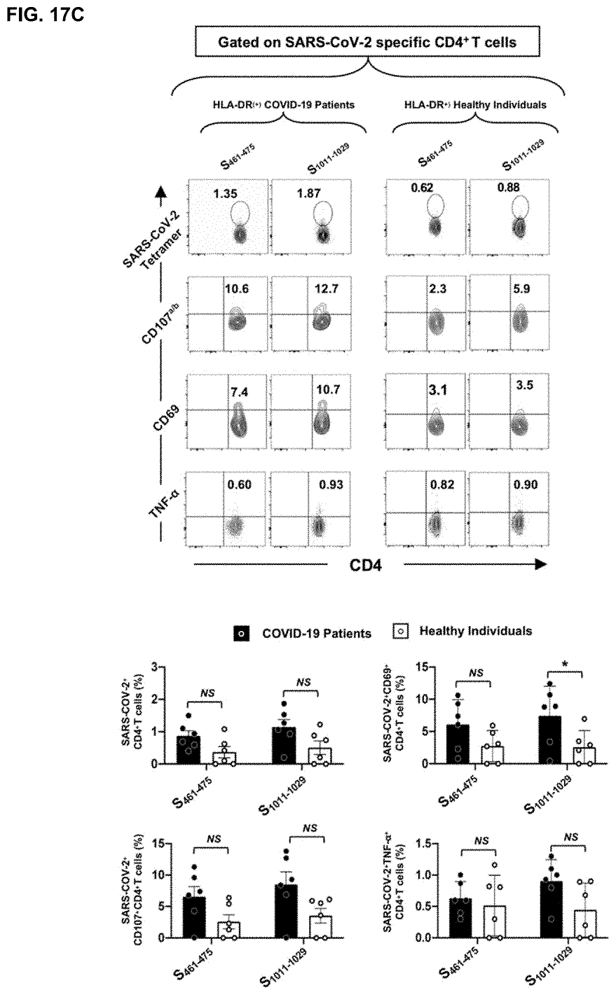

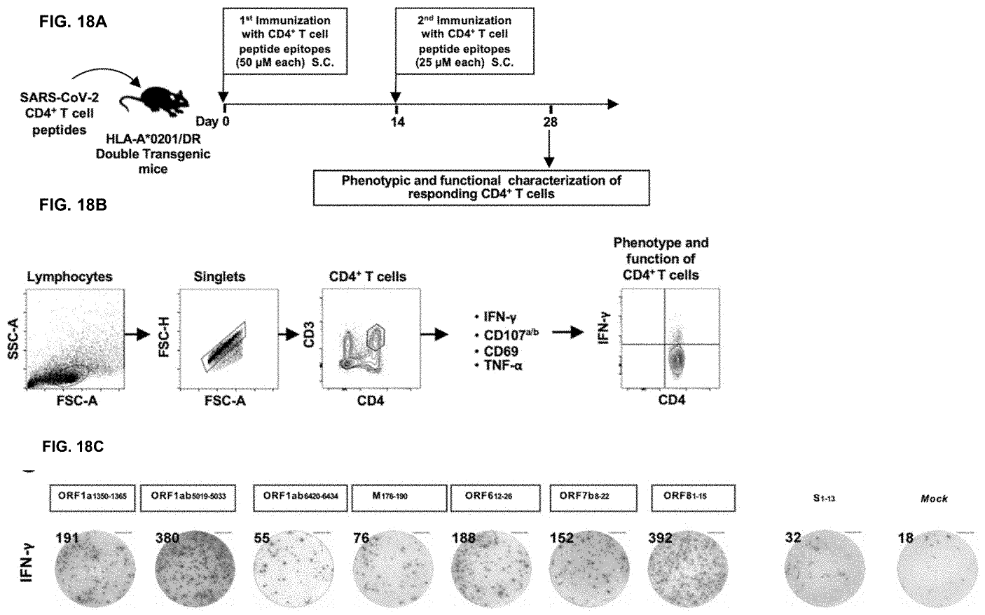

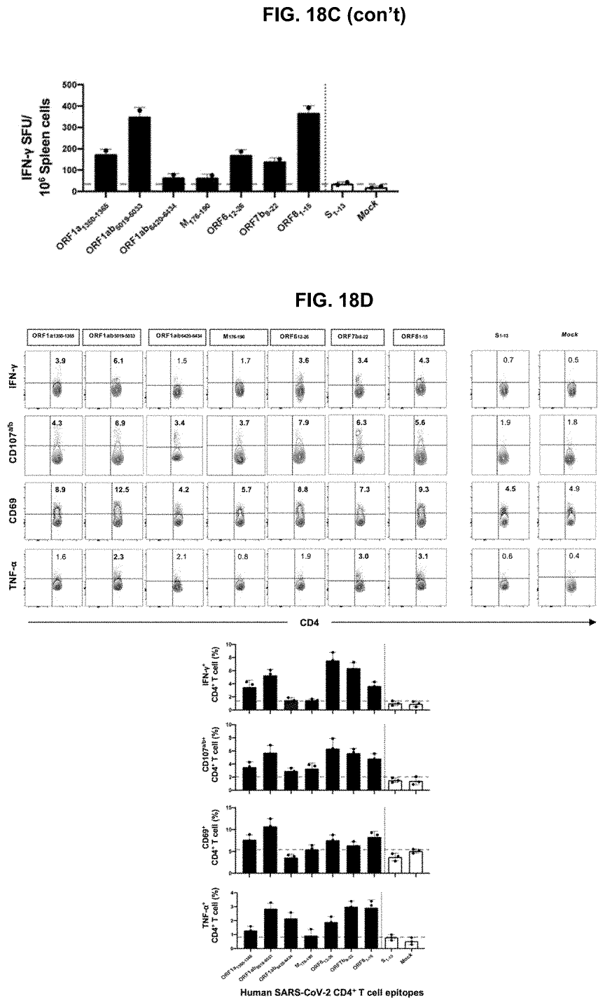

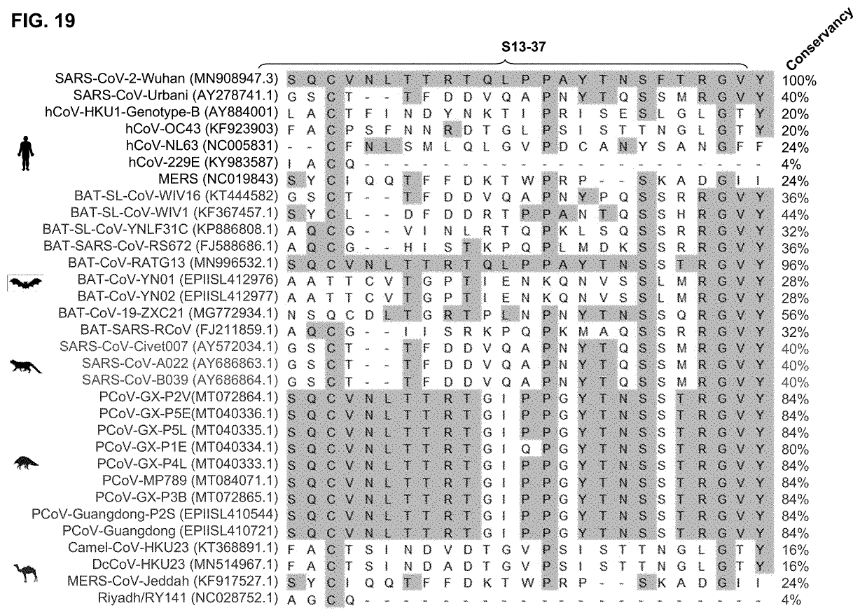

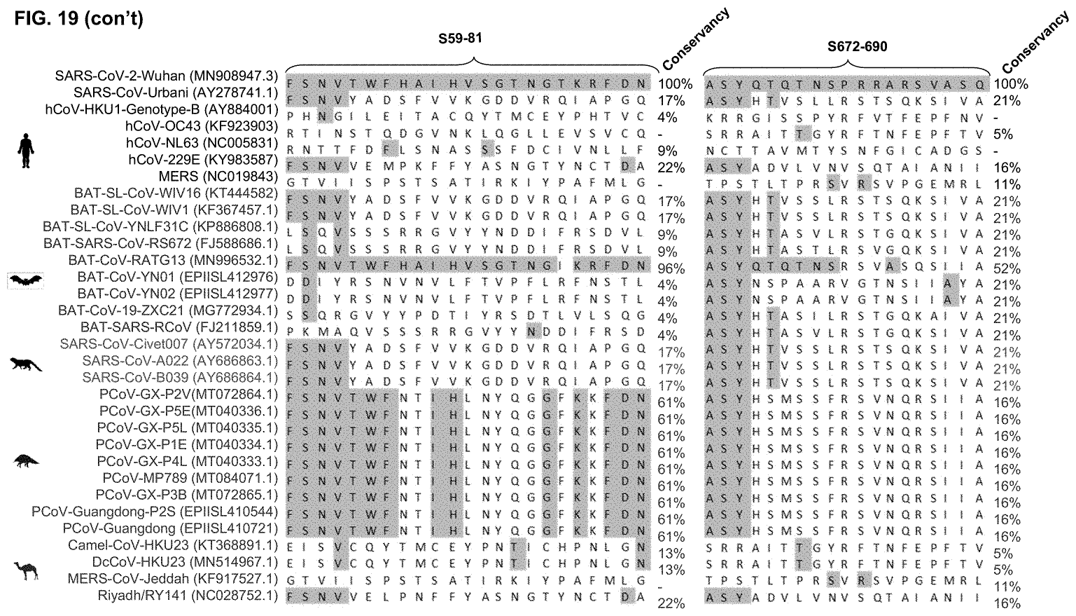

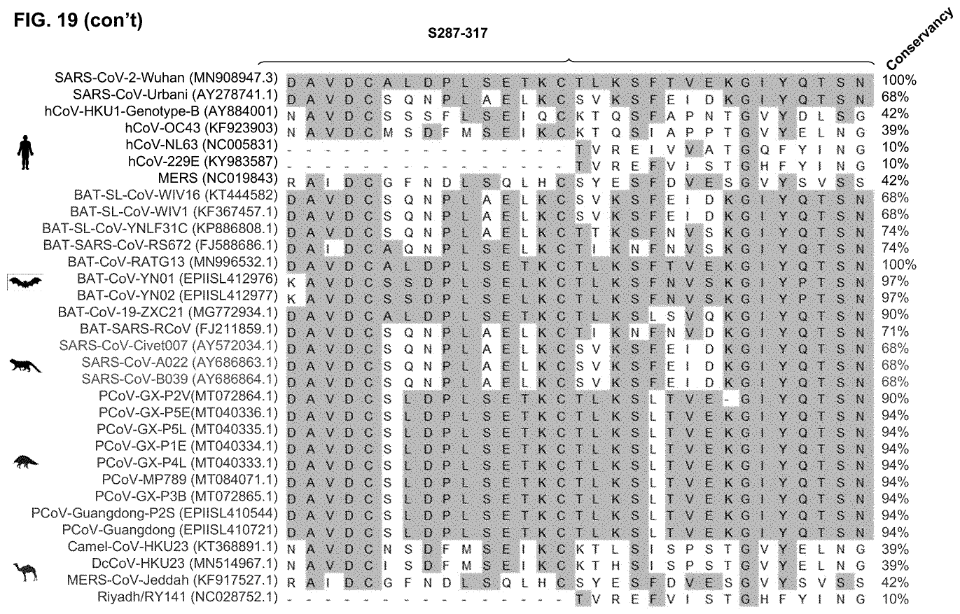

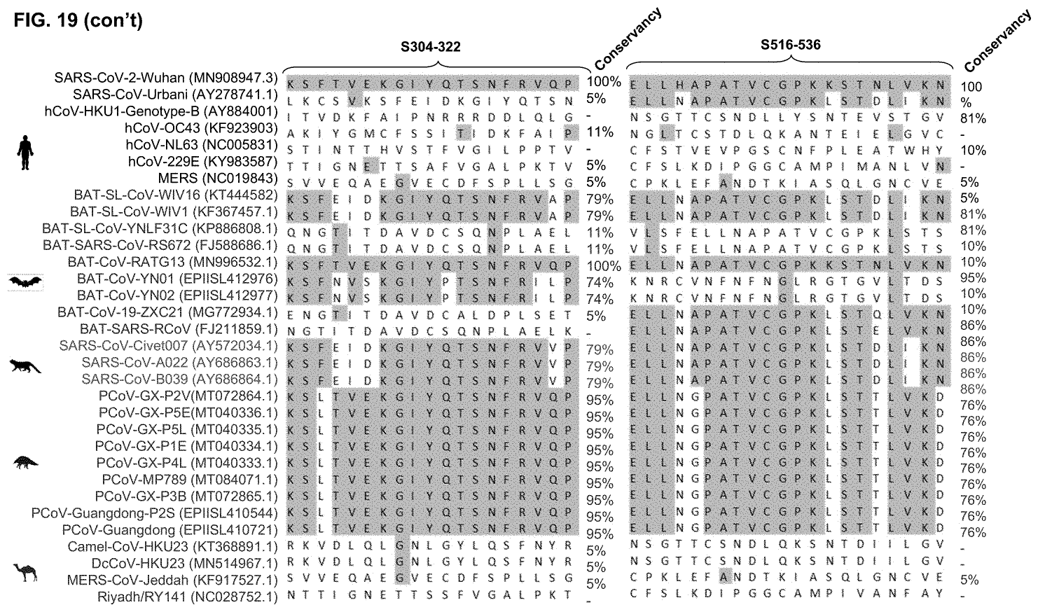

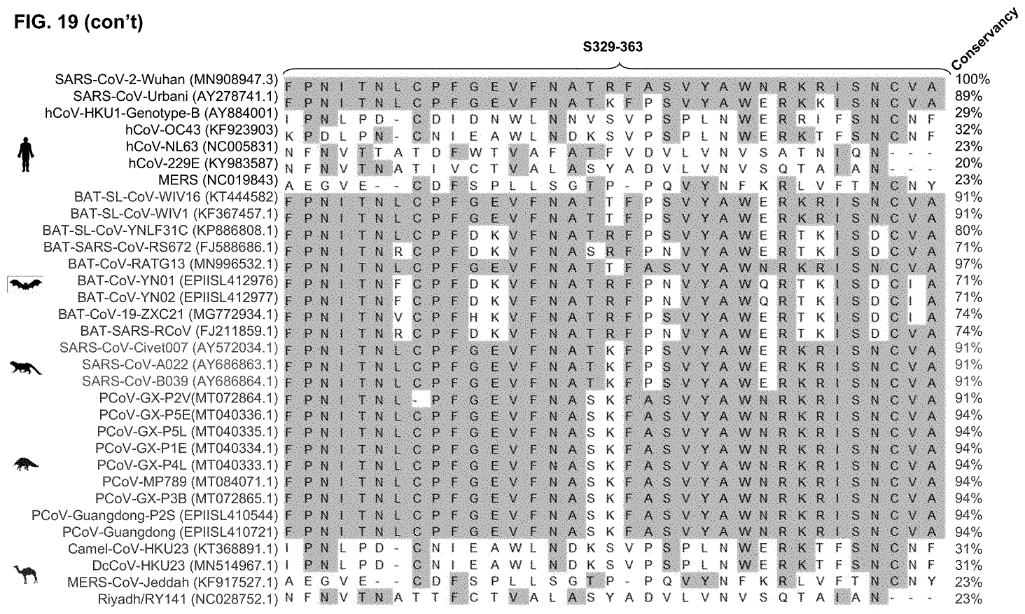

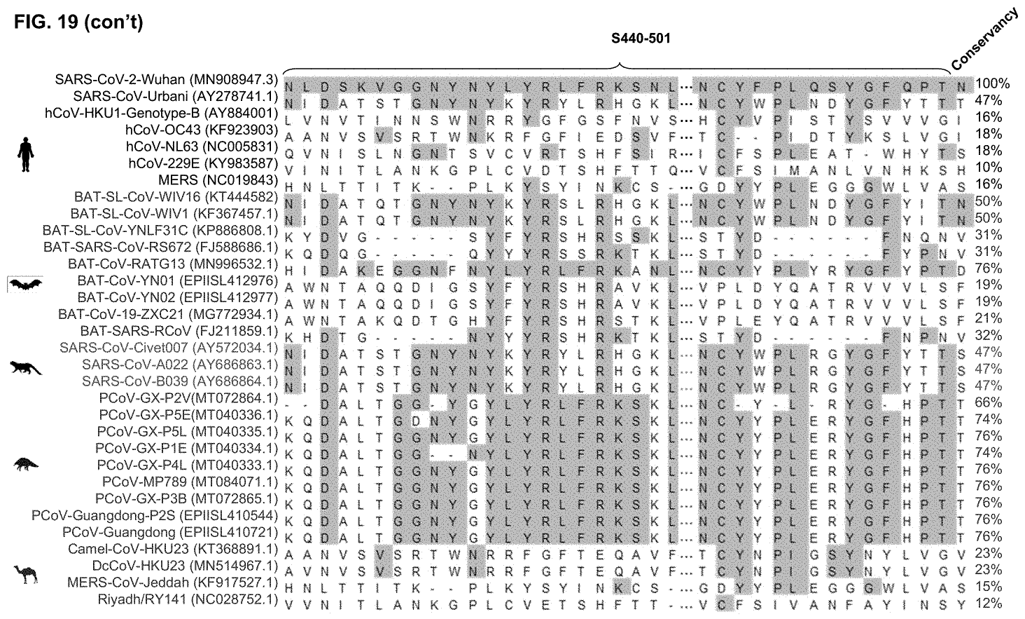

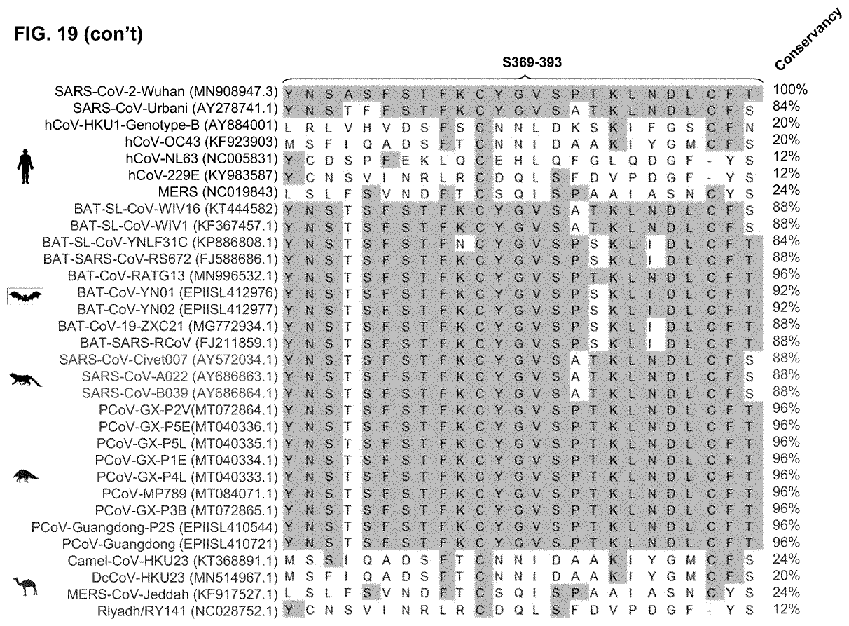

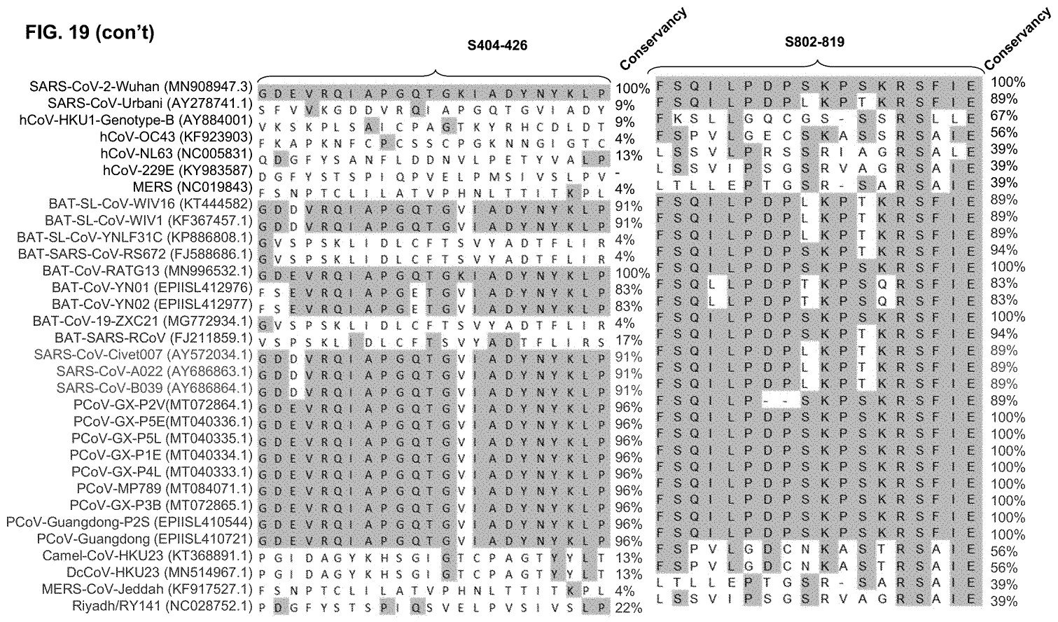

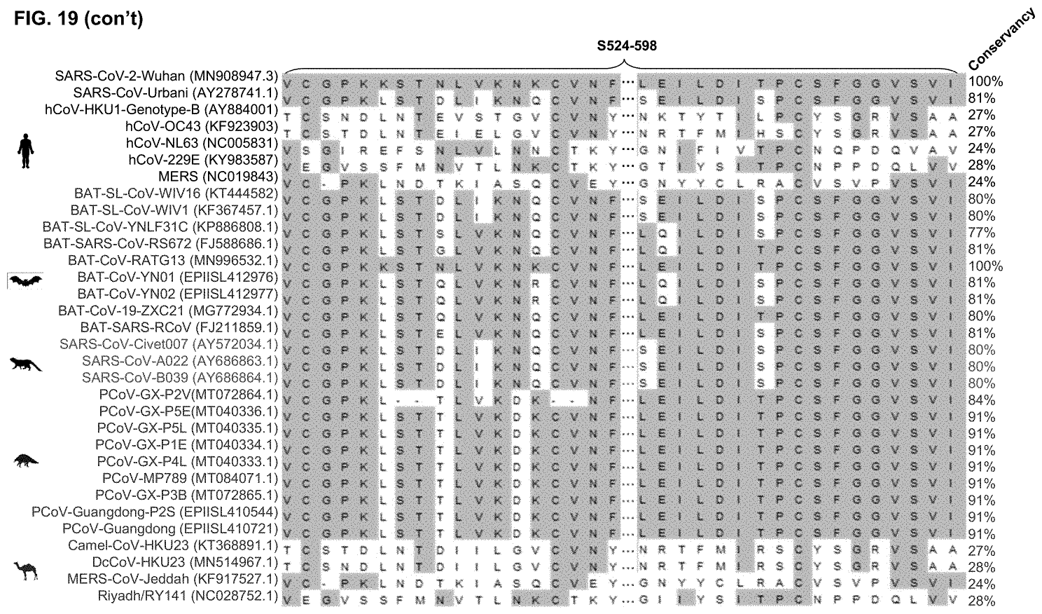

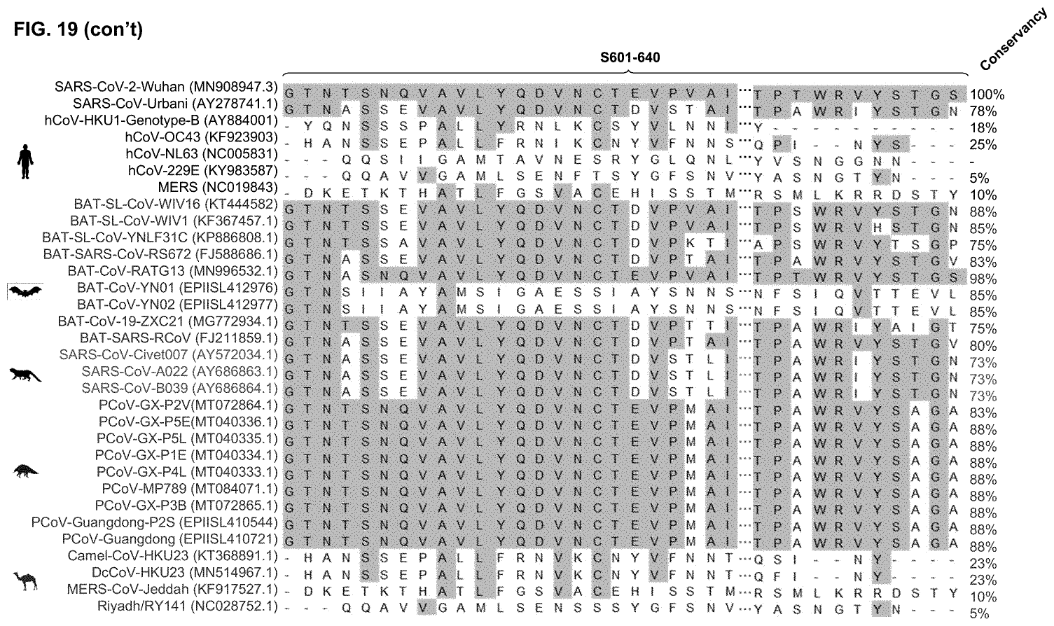

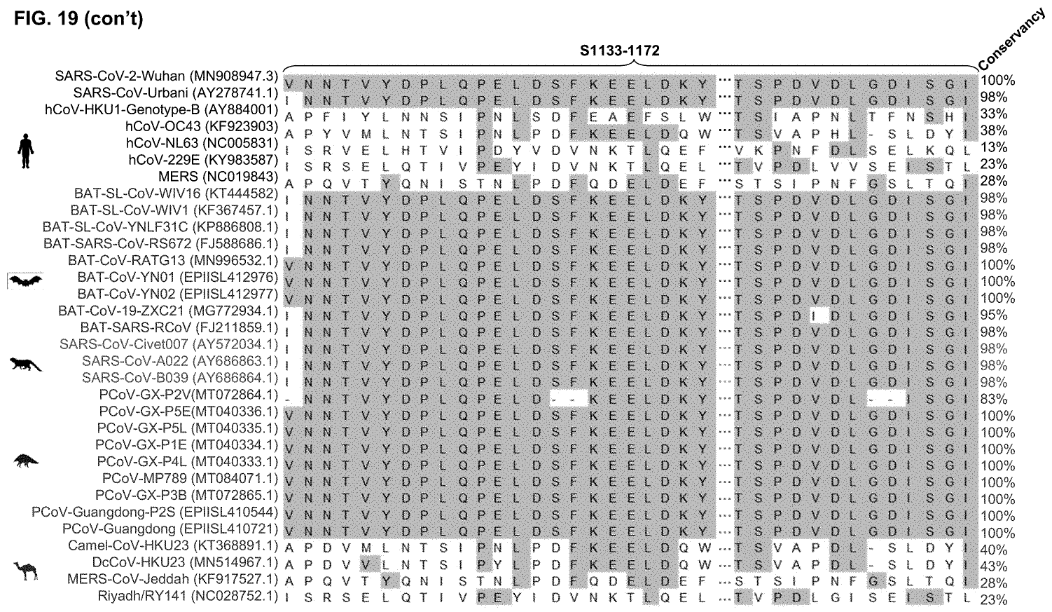

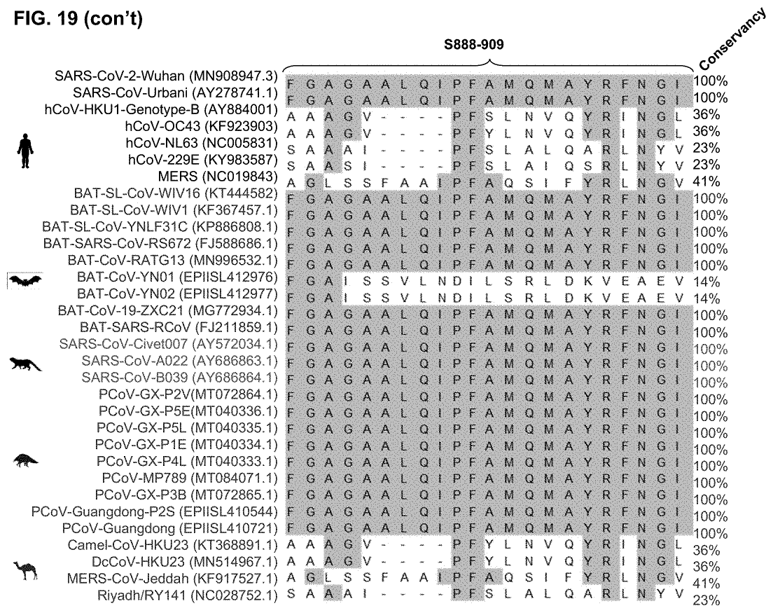

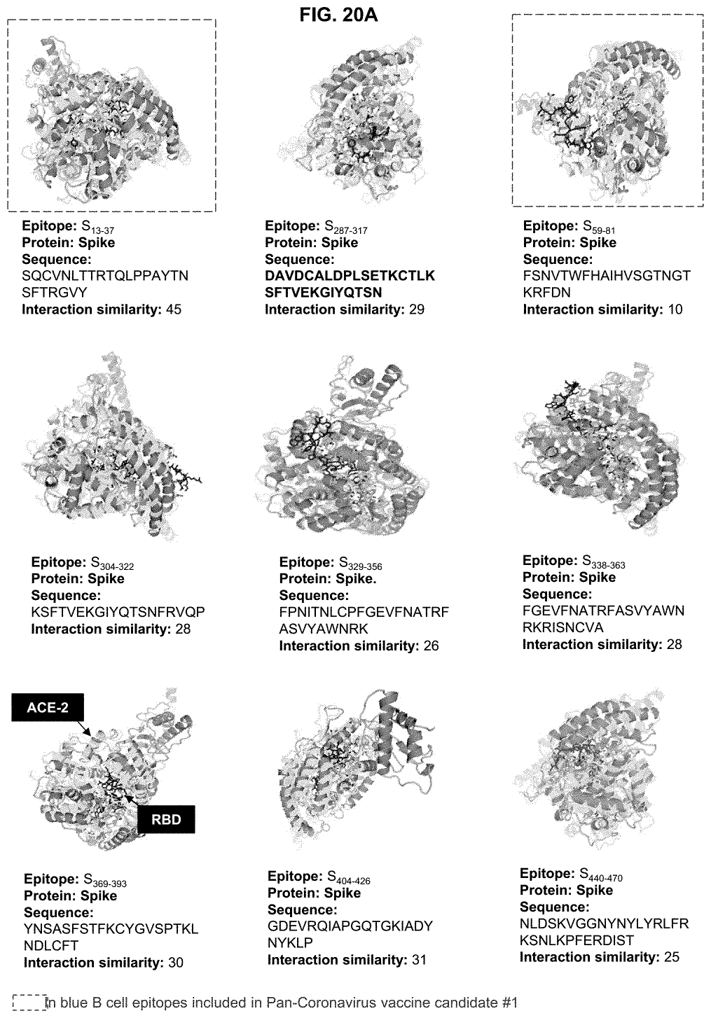

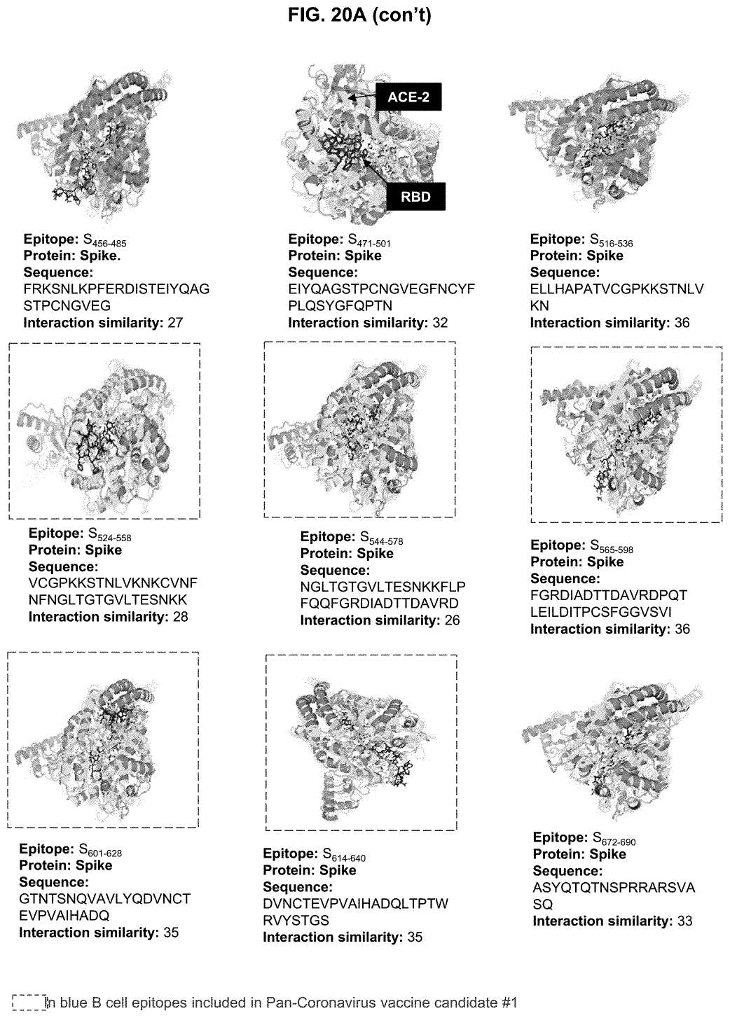

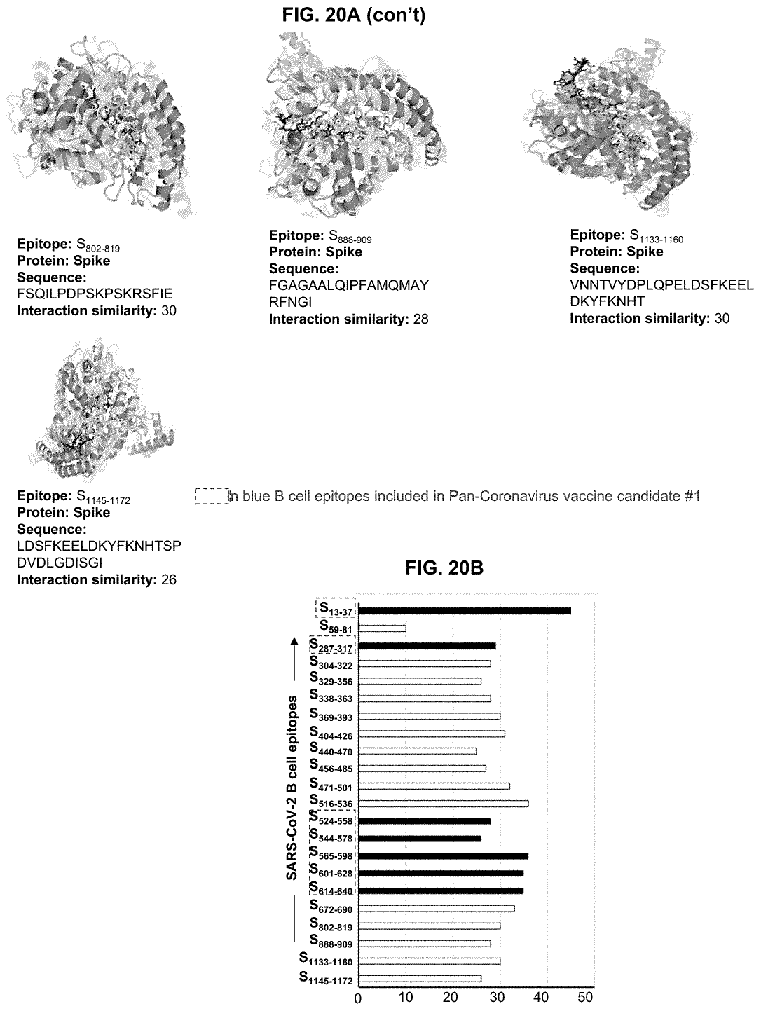

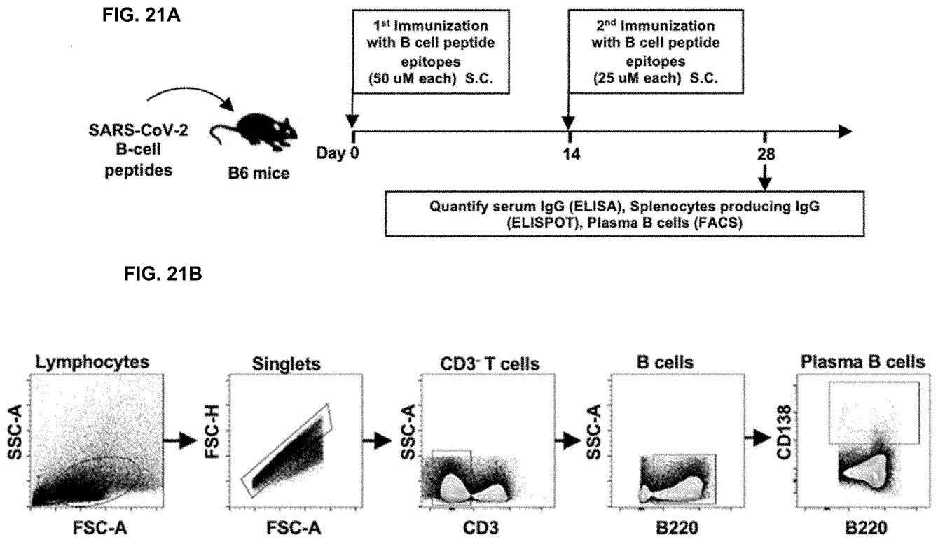

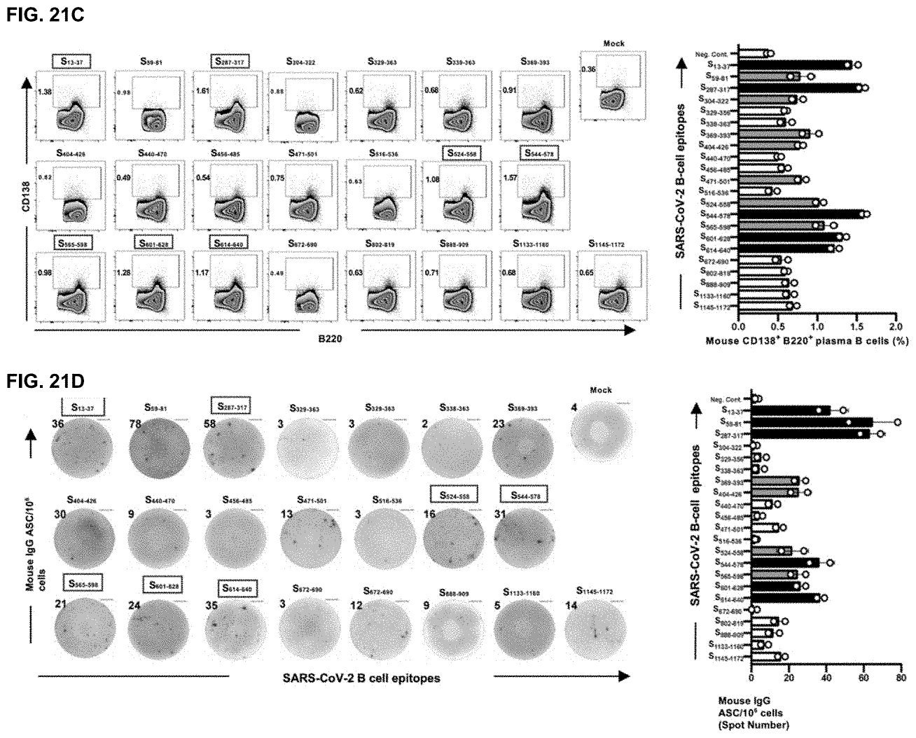

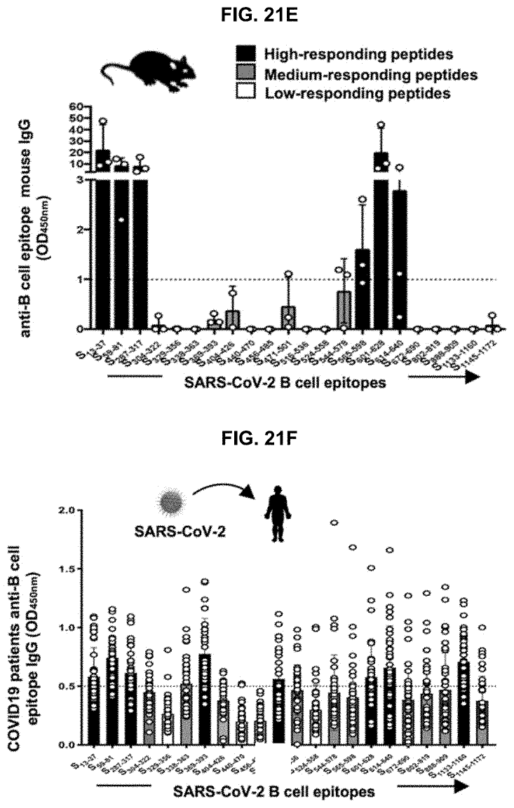

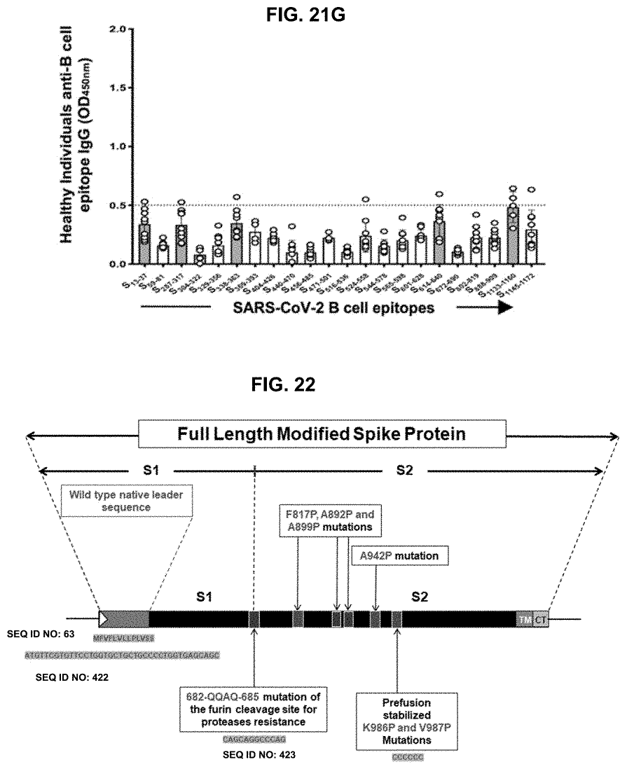

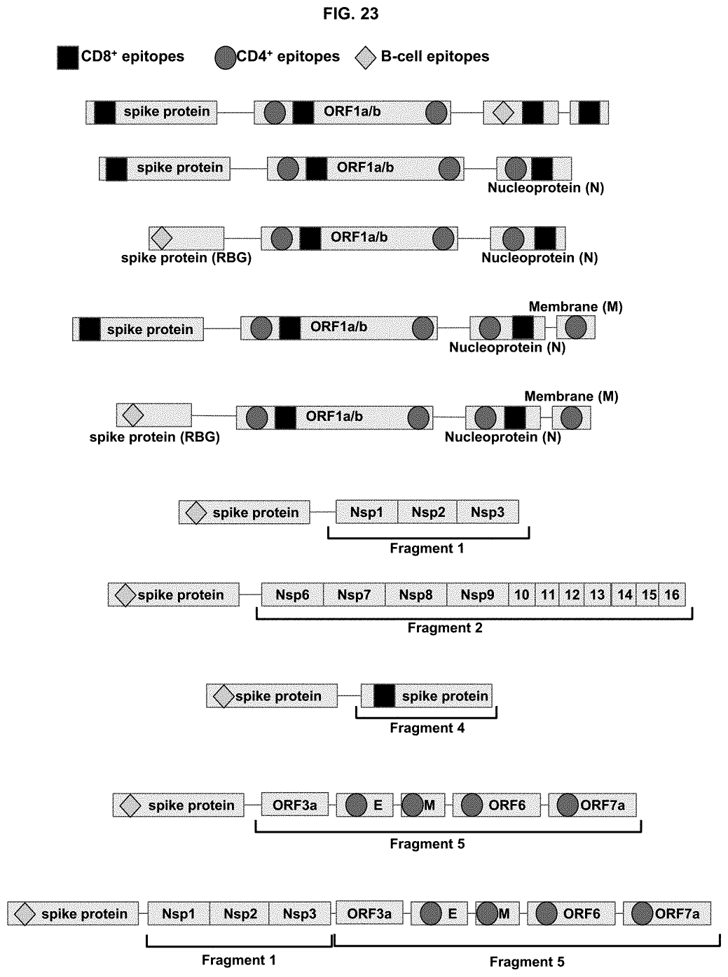

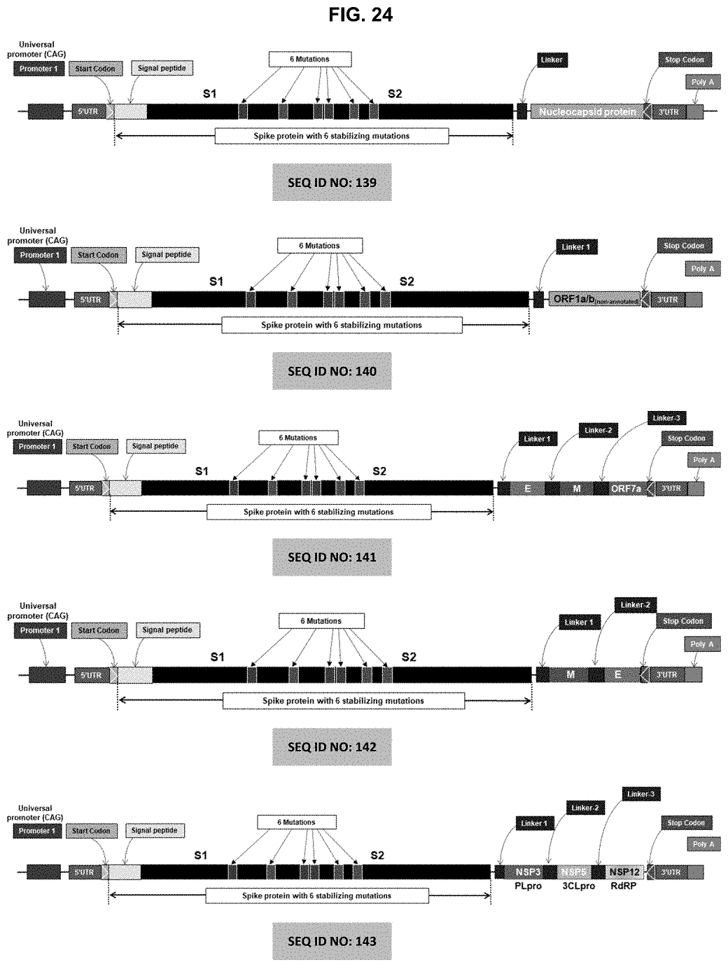

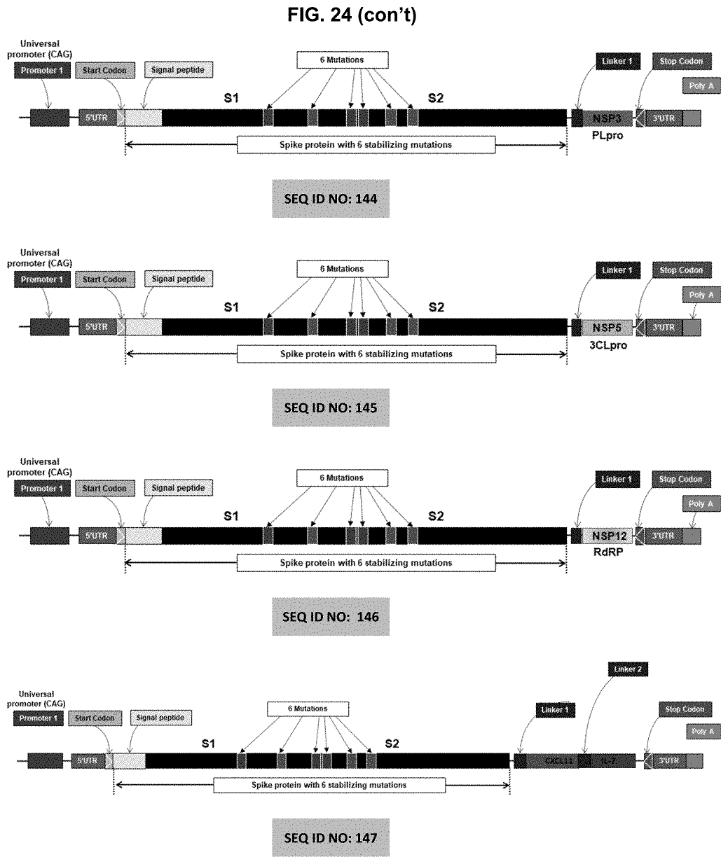

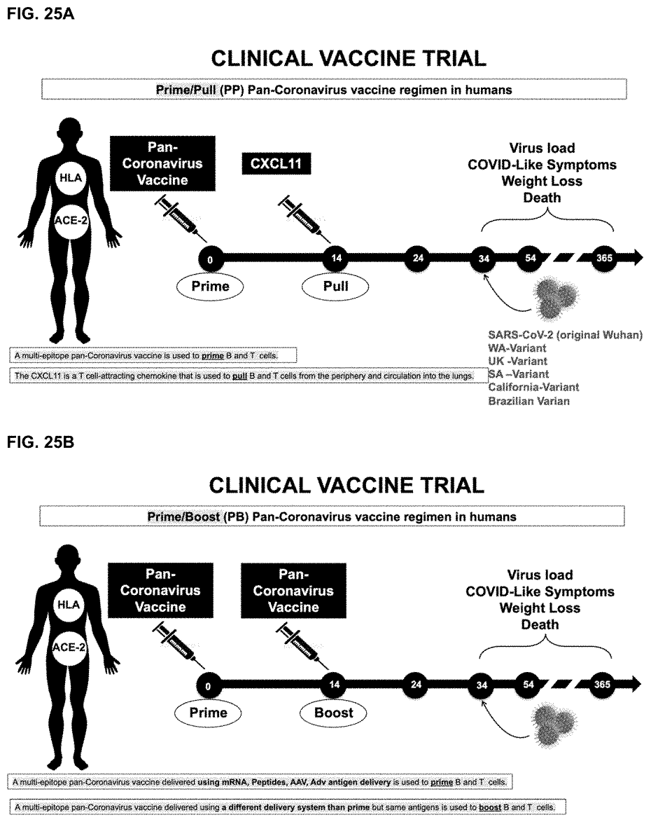

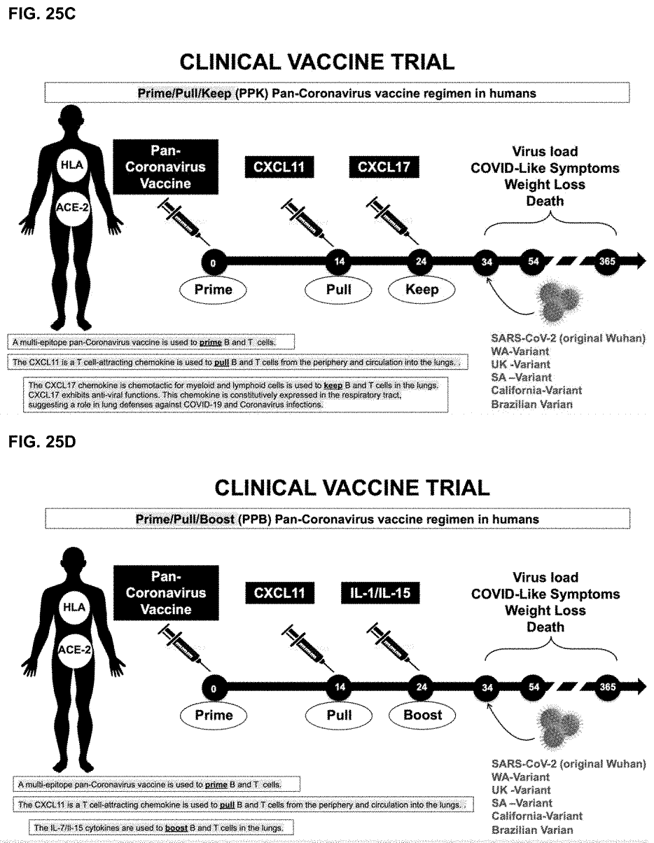

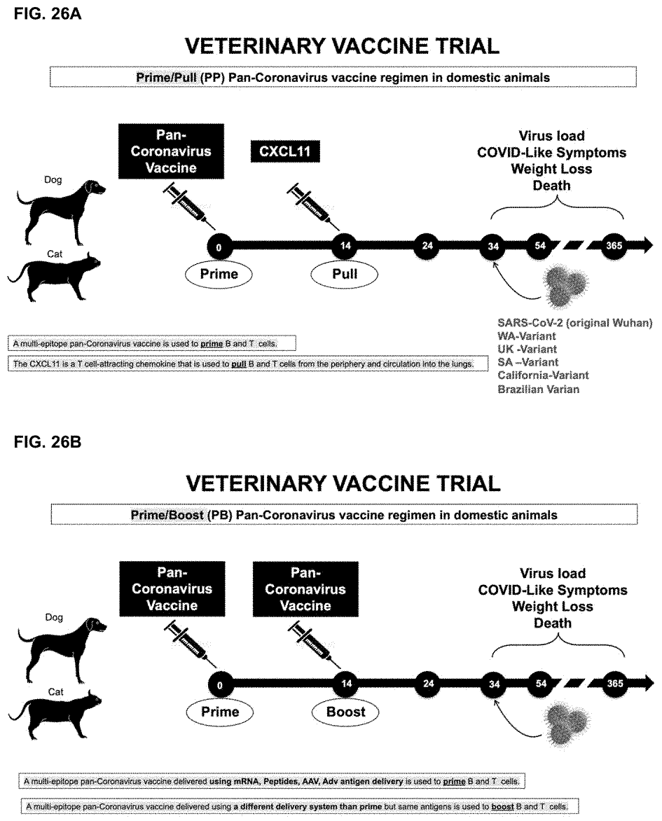

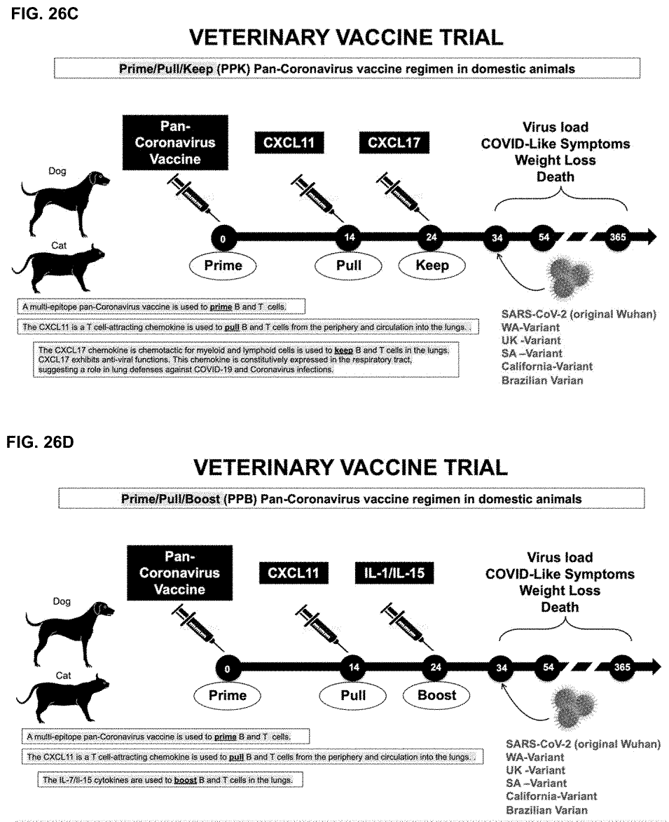

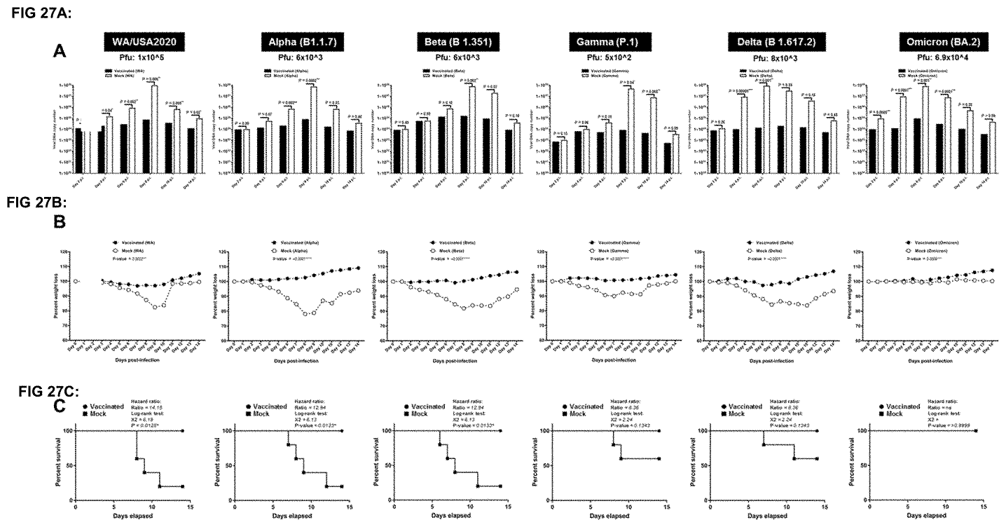

Coronaviruses used for determining conserved large sequences may include human SARS-CoVs as well as animal CoVs (e.g. bats, pangolins, civet cats, minks, camels, etc.) as described herein. As an example, FIG. 2A and FIG. 2B show an evolutionary comparison of genome sequences among beta-coronavirus strains isolated from humans and animals. FIG. 2A shows a phylogenetic analysis performed between SARS-CoV-2 strains (obtained from humans (Homo sapiens (black)), along with the animal's SARS-like Coronaviruses genome sequence (SL-CoVs) sequences obtained from bats (Rhinolophus affinis, Rhinolophus malayanus (red)), pangolins (Manis javanica (blue)), civet cats (Paguma larvata (green)), and camels (Camelus dromedarius (Brown)). The included SARS-CoV/MERS-CoV strains are from previous outbreaks (obtained from humans (Urbani, MERS-CoV, OC43, NL63, 229E, HKU1-genotype-B), bats (WIV16, WIV1, YNLF-31C, Rs672, recombinant strains), camel (Camelus dromedarius, (KT368891.1, MN514967.1, KF917527.1, NC_028752.1), and civet (Civet007, A022, 8039)). The human SARS-CoV-2 genome sequences are represented from six continents. FIG. 2B shows an evolutionary analysis performed among the human-SARS-CoV-2 genome sequences reported from six continents and SARS-CoV-2 genome sequences obtained from bats (Rhinolophus affinis, Rhinolophus malayanus), and pangolins (Manis javanica)).

Additionally, other coronaviruses may be used for determining conserved large sequences (including human SARS-CoVs as well as animal CoVs (e.g., bats, pangolins, civet cats, minks, camels, etc.)) that meet the criteria to be classified as “variants of concern” or “variants of interest.” Coronavirus variants that appear to meet one or more of the undermentioned criteria may be labeled “variants of interest” or “variants under investigation” pending verification and validation of these properties. In some embodiments, the criteria may include increased transmissibility, increased morbidity, increased mortality, increased risk of “long COVID”, ability to evade detection by diagnostic tests, decreased susceptibility to antiviral drugs (if and when such drugs are available), decreased susceptibility to neutralizing antibodies, either therapeutic (e.g., convalescent plasma or monoclonal antibodies) or in laboratory experiments, ability to evade natural immunity (e.g., causing reinfections), ability to infect vaccinated individuals, increased risk of particular conditions such as multisystem inflammatory syndrome or long-haul COVID or increased affinity for particular demographic or clinical groups, such as children or immunocompromised individuals. Once validated variants of interest are renamed “variant of concern” by monitoring organizations, such as the CDC.

The conserved large sequences may be derived from structural (e.g., spike glycoprotein, envelope protein, membrane protein, nucleoprotein) or non-structural proteins of the coronaviruses (e.g., any of the 16 NSPs encoded by ORF1a/b).

In some embodiments, the large sequences are each highly conserved among one or a combination of: SARS-CoV-2 human strains, SL-CoVs isolated from bats, SL-CoVs isolated from pangolin, SL-CoVs isolated from civet cats; and MERS strains isolated from camels. For example, in certain embodiments, the large sequences are each highly conserved among one or a combination of: at least 50,000 SARS-CoV-2 human strains, five SL-CoVs isolated from bats, five SL-CoVs isolated from pangolin, three SL-CoVs isolated from civet cats, and four MERS strains isolated from camels. In certain embodiments, the large sequences are each highly conserved among one or a combination of: at least 80,000 SARS-CoV-2 human strains, five SL-CoVs isolated from bats, five SL-CoVs isolated from pangolin, three SL-CoVs isolated from civet cats, and four MERS strains isolated from camels. In certain embodiments, the large sequences are each highly conserved among one or a combination of: at least 50,000 SARS-CoV-2 human strains in circulation during the COVID-19 pandemic, at least one CoV that caused a previous human outbreak, five SL-CoVs isolated from bats, five SL-CoVs isolated from pangolin, three SL-CoVs isolated from civet cats, and four MERS strains isolated from camels. In certain embodiments, the large sequences are each highly conserved among at least 1 SARS-CoV-2 human strain in current circulation, at least one CoV that has caused a previous human outbreak, at least one SL-CoV isolated from bats, at least one SL-CoV isolated from pangolin, at least one SL-CoV isolated from civet cats, and at least one MERS strain isolated from camels. In certain embodiments, the large sequences are each highly conserved among at least 1,000 SARS-CoV-2 human strains in current circulation, at least two CoVs that has caused a previous human outbreak, at least two SL-CoVs isolated from bats, at least two SL-CoVs isolated from pangolin, at least two SL-CoVs isolated from civet cats, and at least two MERS strains isolated from camels. In certain embodiments, the large sequences are each highly conserved among one or a combination of: at least one SARS-CoV-2 human strain in current circulation, at least one CoV that has caused a previous human outbreak, at least one SL-CoV isolated from bats, at least one SL-CoV isolated from pangolin, at least one SL-CoV isolated from civet cats, and at least one MERS strain isolated from camels. The present invention is not limited to the aforementioned coronavirus strains that may be used to identify conserved large sequences.

In certain embodiments, one or more of the conserved large sequences are derived from one or more SARS-CoV-2 human strains or variants in current circulation; one or more coronaviruses that has caused a previous human outbreak; one or more coronaviruses isolated from animals selected from a group consisting of bats, pangolins, civet cats, minks, camels, and other animal receptive to coronaviruses; and/or one or more coronaviruses that cause the common cold. SARS-CoV-2 human strains and variants in current circulation may include the original SARS-CoV-2 strain (SARS-CoV-2 isolate Wuhan-Hu-1), and several variants of SARS-CoV-2 including but not limited to variant B.1.177 (Spain); variant B.1.160 (Australia), variant B.1.1.7 (UK), variant P.1 (Japan/Brazil), variant B.1,351 (South Africa), variant B.1.427 (California), variant B.1.429 (California), variant B.1.258 (Scotland); variant B.1.221 (Belgium/Netherlands); variant 8.1.367 (Norway/France); variant 8.1.1.277 (UK); variant 8.1.1.302 (Sweden); variant 8.1.525 (North America, Europe, Asia, Africa, and Australia); variant B.1.526 (New York), variant S:677H; variant S:677P; B.1.617.2-Delta, variant 8.1.1.529-Omicron (BA.1); sub-variant Omicron (BA.1); sub-variant Omicron (BA.2); sub-variant Omicron (BA.3); sub-variant Omicron (BA.4); sub-variant Omicron (BA.5). The present invention is not limited to the aforementioned variants of SARS-CoV-2 and encompasses variants identified in the future. The one or more coronaviruses that cause the common cold may include but are not limited to strains 229E (alpha coronavirus), NL63 (alpha coronavirus), OC43 (beta coronavirus), HKU1 (beta coronavirus).

As used herein, the term “conserved” refers to a large sequence that is among the most highly conserved large sequences identified in a sequence alignment and analysis. For example, the conserved large sequences may be the 2 most highly conserved sequences identified. In some embodiments, the conserved large sequences may be the 3 most highly conserved sequences identified. In some embodiments, the conserved large sequences may be the 4 most highly conserved sequences identified. In some embodiments, the conserved large sequences may be the 5 most highly conserved sequences identified. In some embodiments, the conserved large sequences may be the 6 most highly conserved sequences identified. In some embodiments, the conserved large sequences may be the 7 most highly conserved sequences identified, in some embodiments, the conserved large sequences may be the 8 most highly conserved sequences identified. In some embodiments, the conserved large sequences may be the 9 most highly conserved sequences identified. In some embodiments, the conserved large sequences may be the 10 most highly conserved sequences identified. In some embodiments, the conserved large sequences may be the 15 most highly conserved sequences identified. In some embodiments, the conserved large sequences may be the 20 most highly conserved sequences identified. In some embodiments, the conserved large sequences may be the 25 most highly conserved sequences identified. In some embodiments, the conserved large sequences may be the 30 most highly conserved sequences identified. In some embodiments, the conserved large sequences may be the 40 most highly conserved sequences identified. In some embodiments, the conserved large sequences may be the 50 most highly conserved sequences identified. In some embodiments, the conserved sequences may be the 50% most highly conserved large sequences identified. In some embodiments, the conserved large sequences may be the 60% most highly conserved sequences identified. In some embodiments, the large conserved sequences may be the 70% most highly conserved sequences identified. In some embodiments, the conserved large sequences may be the 80% most highly conserved sequences identified. In some embodiments, the conserved large sequences may be the 90% most highly conserved sequences identified. In some embodiments, the conserved large sequences may be the 95% most highly conserved sequences identified. In some embodiments, the conserved large sequences may be the 99% most highly conserved sequences identified. The present invention is not limited to the aforementioned thresholds.

FIG. 3A shows an example of a systems biology approach utilized in the present invention.

In some embodiments, the composition comprises one or more large sequences. In some embodiments, the one or more large sequences comprises at least one of one or more conserved coronavirus B-cell target epitopes; one or more conserved coronavirus CD4⁺ T cell target epitopes; and one or more conserved coronavirus CD8⁺ T cell target epitopes

In other embodiments, the vaccine composition comprises two or more large sequences. In some embodiments, the two or more large sequences comprises at least one of one or more conserved coronavirus B-cell target epitopes; one or more conserved coronavirus CD4⁺ T cell target epitopes; and one or more conserved coronavirus CD8⁺ T cell target epitopes

In some embodiments, the large sequences comprise one or more conserved coronavirus B-cell target epitopes and one or more conserved coronavirus CD4⁺ T cell target epitopes. In some embodiments, the large sequences comprise one or more conserved coronavirus B-cell target epitopes and one or more conserved coronavirus CD8⁺ T cell target epitopes. In some embodiments, the large sequences comprise one or more conserved coronavirus CD8⁺ target epitopes and one or more conserved coronavirus CD4⁺ T cell target epitopes. In some embodiments, the large sequences comprise one or more conserved coronavirus CD8⁺ target epitopes. In some embodiments, the large sequences comprise one or more conserved coronavirus CD4⁺ target epitopes. In some embodiments, the large sequences comprise one or more conserved coronavirus B cell target epitopes.

In some embodiments, the vaccine composition comprises one or more conserved coronavirus CDB8⁺ target epitopes. In some embodiments, the vaccine composition comprises one or more conserved coronavirus CD4⁺ target epitopes. In some embodiments, the vaccine composition comprises one or more conserved coronavirus B cell target epitopes.

As will be discussed herein, in certain embodiments, the vaccine composition comprises whole spike protein, one or more coronavirus CD4+ T cell target epitopes; and one or more coronavirus CD8+ T cell target epitopes. In certain embodiments, the vaccine composition comprises at least a portion of the spike protein (e.g., wherein the portion comprises a trimerized SARS-CoV-2 receptor-binding domain (RBD)), one or more coronavirus CD4+ T cell target epitopes; and one or more coronavirus CD8+ T cell target epitopes. In some embodiments, the one or more coronavirus CD4+ T cell target epitopes; and one or more coronavirus CD8+ T cell target epitopes may be in the form of a large sequence.

The large sequences may be each separated by a linker. In certain embodiments, the linker allows for an enzyme to cleave between the large sequences. The present invention is not limited to particular linkers or particular lengths of linkers. As an example, in certain embodiments, one or more large sequences may be separated by a linker 2 amino acids in length or a linker 3 amino acids in length, or a linker 4 amino acids in length, or a linker 5 amino acids in length, or a linker 6 amino acids in length, or a linker 7 amino acids in length, or a linker 8 amino acids in length, or a linker 9 amino acids in length, or a linker 10 amino acids in length. In certain embodiments, one or more large sequences may be separated by a linker from 2 to 10 amino acids in length.

Linkers are well known to one of ordinary skill in the art. Non-limiting examples of linkers include AAY, KK, and GPGPG.

The large sequences may be derived from structural proteins, non-structural proteins, or a combination thereof. For example, structural proteins may include spike proteins (S), envelope proteins (E), membrane proteins (M), or nucleoproteins (N).

In some embodiments, the large sequences are derived from at least one SARS-CoV-2 protein. The SARS-CoV-2 proteins may include ORF1ab protein, Spike glycoprotein, ORF3a protein, Envelope protein, Membrane glycoprotein, ORF6 protein, ORF7a protein, ORF7b protein, ORF8 protein, Nucleocapsid protein, and ORF10 protein. The ORF1ab protein provides nonstructural proteins (Nsp) such as Nsp1, Nsp2, Nsp3 (Papain-like protease), Nsp4, Nsp5 (3C-like protease), Nsp6, Nsp7, Nsp8, Nsp9, Nsp10, Nsp11, Nsp12 (RNA polymerase), Nsp13 (5′ RNA triphosphatase enzyme), Nsp14 (guanosineN7-methyltransferase), Nsp15 (endoribonuclease), and Nsp16 (2′O-ribose-methyltransferase).

The SARS-CoV-2 has a genome length of 29,903 base pairs (bps) ssRNA (SEQ ID NO: 1). Generally, the region between 266-21555 bps codes for ORF1ab polypeptide; the region between 21563-25384 bps codes for one of the structural proteins (spike protein or surface glycoprotein); the region between 25393-26220 bps codes for the ORF3a gene; the region between 26245-26472 bps codes for the envelope protein; the region between 26523-27191 codes for the membrane glycoprotein (or membrane protein); the region between 27202-27387 bps codes for the ORF6 gene: the region between 27394-27759 bps codes for the ORF7a gene; the region between 27894-28259 bps codes for the ORF8 gene; the region between 28274-29533 bps codes for the nucleocapsid phosphoprotein (or the nucleocapsid protein); and the region between 29558-29674 bps codes for the ORF10 gene.

The large sequences may comprise a T-cell epitope restricted to a large number of human class 1 and class 2 HLA haplotypes and not restricted to HLA-0201 for class 1 or HLA-DR for class 2. The conserved large sequences may be restricted to human HLA class 1 and 2 haplotypes. In some embodiments, the conserved epitopes are restricted to cat and dog MHC class 1 and 2 haplotypes.

### Large Sequences

The antigen may comprise large sequences, such as conserved large sequences that are highly conserved among human and animal coronaviruses. As used herein, the term large sequence refers to a sequence having at least 25 amino acids or at least 75 nucleotides. The large sequences comprise epitopes, such as the conserved epitopes described herein.

In some embodiments, the large sequence has at least 75 nt. In some embodiments, the large sequence has at least 150 nt. In some embodiments, the large sequence has at least 200 nt. In some embodiments, the large sequence has at least 250 nt. In some embodiments, the large sequence has at least 300 nt. In some embodiments, the large sequence has at least 400 nt. In some embodiments, the large sequence has at least 500 nt. In some embodiments, the large sequence has at least 600 nt. In some embodiments, the large sequence has at least 700 nt. In some embodiments, the large sequence has at least 800 nt. In some embodiments, the large sequence has at least 900 nt. In some embodiments, the large sequence has at least 1000 nt. In some embodiments, the large sequence has at least 1500 nt. In some embodiments, the large sequence has at least 2000 nt. In some embodiments, the large sequence has at least 2500 nt. In some embodiments, the large sequence has at least 3000 nt. In some embodiments, the large sequence has at least 3500 nt. In some embodiments, the large sequence has at least 4000 nt. In some embodiments, the large sequence has at least 4500 nt. In some embodiments, the large sequence has at least 5000 nt.

In order to identify the conserved large sequences, sequence alignments and analysis were performed as described herein as well as below.

Sequence comparison among SARS-CoV-2 and previous coronavirus strains: Sequence homology analysis we performed and compare the Severe acute respiratory syndrome coronavirus 2 (SARS-CoV-2) isolate Wuhan-Hu-1, to complete genome with sequences of SARS-CoV-2 variants, common cold corona virus strains (HKU1 genotype B, CoV-OC43, CoV-NL63, and CoV-229E), SARS-CoV-Urbani, MERS and coronavirus strains from bats (Rhinolophus affinis and R. malayanus), pangolin (Manis javanica), civet cats (Paguma larvata), and camel (Camelus dromedarius and C. bactrianus).

The human SARS-CoV-2 variant genome sequences were retrieved from the GISAID database, representing major Variants of Concern which are known for their high degree of transmissibility and pathogenicity. The sequences used in this study are 20A.EU1 from Spain (EPI_ISL_691726-hCoV-19-VOC-20A.EU1), 20A.EU2 from Australia (EPI_ISL_418799-hCoV-19-VOC-20A.EU2), B.1.1.7 from England (EPI_ISL_581117-hCoV-19-VOC-B.1.1.7), B.1.351 from South Africa (EPI_ISL_660615-hCoV-19-VOC-B.1.351), P.1 from Brazil (EPI_ISL_581117-hCoV-19-VOC-P.1), CAL.20C from California (EPI_ISL_730092-hCoV-19-VOC-B.1.427/B.1.429), B.1.258 from Scotland (EPI_ISL_858559-hCoV-19-VOC-B.1.258), B.1.221 from Belgium/Netherlands (EPI_ISL_734790-hCoV-19-VOC-B.1.221), B.1.367 from Norway/France (EPI_ISL_541518-hCoV-19-VOC-B.1.367), B.1.1.277 from Netherlands/Denmark/UK (EPI_ISL_500783-hCoV-19-VOC-B.1.1.277), B.1.1.302 from Sweden (EPI_ISL_717929-hCoV-19-VOC-B.1.1.302). Similarly, HKU1 genotype B (AY884001), CoV-OC43 (KF923903), CoV-NL63 (NC_005831), and CoV-229E (KY983587), SARS-CoV-Urbani (AY278741.1), MERS (NC_019843).

Bat CoV strains used in this analysis include strains RaTG13 (MN996532.2), Rs672/2006 (FJ588686.1), YNLF_31C (KP886808.1), WIV1 (KF367457.1), WIV16 (KT444582.1), ZXC21 (MG772934.1), RmYN02 (EPI_ISL_412977), bat-RmYN01 (EPI_ISL_412976), MERS-Bat-CoV/P. khulii/Italy/206645-63/2011 (MG596803.1). More-so, five genome sequences representing Pangolin (MT040333.1-PCoV_GX-P4L, MT040334.1-PCoV_GX-P1E, MT040335.1-PCoV_GX-P5L, MT040336.1-PCoV_GX-P5E, MT072864.1-PCoV_GX-P2V, MT121216.1-PCoV-MP789), three Civet cat specific genome sequences (AY572034.1, AY686864.1, AY686863.1), and four CoV sequences from camels (NC_028752.1, KF917527.1, MN514967.1, KT368891.1) were included in this sequence homology analysis aimed at evaluating the most conserved regions in different structural and non-structural proteins in CoV genome. These sequences were obtained either from National Center for Biotechnology Information (NCBI) or Global initiative on sharing all influenza data (GISAID). For phylogenetic analyses, SARS-CoV-2 full-genome sequences were aligned with CLUSTAL W using MEGAX. All the SARS-CoV-2 sequences were compared to existing genomes using online NCBI BLAST.

Determination of SARS-COV-2 Sequence Conservation: Each Wuhan-Hu-1 (GeneBank: NC_045512.2) specific structural (Spike glycoprotein (YP_009724390.1), Membrane protein (YP_009724393.1), Envelope protein (YP_009724392.1), Nucleocapsid phosphoprotein (YP_009724397.2)), and non-structural proteins (ORF1a/b polyprotein (YP_009724389.1), ORF3a (YP_009724391.1), ORF6 (YP_009724394.1), ORF7a (YP_009724395.1), ORF7b (YP_009725318.1), ORF8 (YP_009724396.1), and ORF10 (YP_009725255.1)) protein sequences were compared against the consensus protein sequences from SARSCoV and MERS-CoV and the protein sequences from closest relative cross species CoV strains using the Nucleotide BLAST (blastn) algorithm to compute the pairwise identity between Wuhan-Hu-1 proteins and their comparison target.

Further as the present invention is interested in the highly similar sequences across CoV strains, megablast was performed. For each of the queried sequences, Query coverage, E value. Percent identity were determined. The queried homology obtained against one bat CoV strain RmYN01, which was found earlier to be phylogenetically less similar to SARS-CoV-2, but has more genetic similarities with SARS-CoV-Urbani was taken as a standard to ascertain the homologous sequences across CoV strains. The strategy was helpful to find out how genetically more conserved regions among different CoVs. This sequence has a query coverage of 59%, and a percent identity of 78.73% when compared against the SARS-CoV-2 genome sequence. It has five matched regions which further showed sequence homology among other CoVs as well. Matched region 1 spanned between 1 bp-1580 bp (fragment) showed sequence homology with nsp1 (leader protein), nsp2, and nsp3, whereas matched region 2 spanned between 3547 bp-7096 bp (fragment 2) showed sequence homology with multiple subunits of ORF1a/b like 3CLpro, nsp6, nsp7, nsp8, nsp9, nsp10, RNA dependent RNA polymerase, helicase, nsp14, nsp15, and nsp16. Interestingly, a major region spanning in the non-annotated region of the ORF1a/b between 17472 bp-21156 bp (fragment 3) also showed sequence identity. The fourth stretch of sequence identity spanned through 22584 bp-24682 bp (fragment 4) covering a section of the Spike glycoprotein, that importantly covers the major Receptor Binding Domain in the SARS-CoV-2 as well. The last segment of the homologous sequence showed percent identity with regions specific to the ORF3a, Envelope protein, Membrane protein, ORF6, and ORF7a (26193 bp-27421 bp; fragment 5).

In some embodiments, five fragments from the SARS-CoV-2 Wuhan Strain were found to be highly conserved (1 bp-1580 bp (fragment 1), 3547 bp-12830 bp (fragment 2), 17472 bp-21156 bp (fragment 3), 22584 bp-24682 bp (fragment 4), and 26193 bp-27421 bp (fragment 5). Next, each fragment underwent another round of sequence homology analysis.

In some embodiments, the vaccine composition comprises one large sequence. In some embodiments, the vaccine composition comprises one or more large sequences. In some embodiments, the vaccine composition comprises two or more large sequences. In some embodiments, the vaccine composition comprises three or more large sequences. In some embodiments, the vaccine composition comprises four or more large sequences. In some embodiments, the vaccine composition comprises five or more large sequences, e.g., 5, 6, 7, 8, etc.

In some embodiments, the large sequences are derived from a whole protein sequence expressed by SARS-CoV-2. In other embodiments, large sequences are derived from a partial protein sequence expressed by SARS-CoV-2. In some embodiments, the large sequence of said proteins comprise B cell epitopes and T-cell epitopes that are restricted to a large number, e.g., from 3 to 10, different haplotypes that encompass 100% of the population regardless of race and ethnicity) of human class 1 and class 2 HLA haplotypes, so they are not restricted only to HLA-0201 for class 1 or HLA-DR1 for class 2.

As previously discussed, the large sequences may be highly conserved among human and animal coronaviruses. In some embodiments, the large sequences are derived from one or a combination of: one or more SARS-CoV-2 human strains or variants in current circulation; one or more coronaviruses that has caused a previous human outbreak; one or more coronaviruses isolated from animals selected from a group consisting of bats, pangolins, civet cats, minks, camels, and other animal receptive to coronaviruses; and/or one or more coronaviruses that cause the common cold.

As previously discussed, the SARS-CoV-2 human strains or variants in current circulation may include variant 6.1.177; variant B.1.160, variant 6.1.1.7 (UK), variant P.1 (Japan/Brazil), variant B.1.351 (South Africa), variant 8.1.427 (California), variant 6.1.429 (California), variant B.1.258; variant B.1.221; variant B.1.367; variant B.1.1.277; variant B.1.1.302; variant B.1.525; variant 6.1.526, variant S:677H; variant S:677P; B.1.617.2-Delta, variant B.1.1.529-Omicron (BA.1); sub-variant Omicron (BA.1); sub-variant Omicron (BA.2); sub-variant Omicron (BA.3); sub-variant Omicron (BA.4); sub-variant Omicron (BA.5). In some embodiments, the one or more coronaviruses that cause the common cold are selected from 229E alpha coronavirus, NL63 alpha coronavirus, OC43 beta coronavirus, and HKU1 beta coronavirus.

The large sequence(s) may be derived from structural proteins, non-structural proteins, or a combination thereof. The large sequence(s) may be selected from ORF1ab protein, Spike glycoprotein (e.g., the RBD), ORF3a protein, Envelope protein, Membrane glycoprotein, ORF6 protein, ORF7a protein, ORF7b protein, ORF8 protein, Nucleocapsid protein, and/or an ORF10 protein. Note the ORF1ab protein comprises nonstructural protein (Nsp) 1, Nsp2, Nsp3, Nsp4, Nsp5, Nsp6, Nsp7, Nsp8, Nsp9, Nsp10, Nsp11, Nsp12, Nsp13, Nsp14, Nsp15 and Nsp16.

In some embodiments, a large sequence comprises conserved fragments from over 150,000 CoV strains circulating in the majority of countries around the world (Table 1, FIG. 4). In some embodiments, fragment 1 comprises the base pairs 1-1580. In some embodiments, fragment 1 may comprise the proteins Nsp1, Nsp2, and Nsp3 as well as unannotated regions (FIG. 5). In some embodiments, fragment 2, comprises the base pairs 3547-12830. In some embodiments, fragment 2 may comprise the proteins Nsp5, Nsp6, Nsp7, Nsp8, Nsp9, Nsp10, Nsp11, Nsp12, Nsp13, Nsp14, Nsp15, Nsp16, as well as unannotated regions (FIG. 6). In some embodiments, fragment 3 comprises the base pairs 17472-21156. In some embodiments, fragment 3 comprises unannotated regions (FIG. 7). In some embodiments, fragment 4 comprises the base pairs 22584-24682. In some embodiments, fragment 4 comprises the spike glycoprotein (FIG. 8). In some embodiments, fragment 5 comprises the base pairs 26193-27421. In some embodiments, fragment 5 comprises the proteins ORF3a, Envelope (E), Membrane (M), ORF6, ORF7a, as well as unannotated regions (FIG. 9).

TABLE 1

| | | | SEQ |
|---|---|---|---|
| Fragment | | | ID |
| No: | Proteins | Sequence | NO: |
| 1 | Nspt | GACGTGCTAGTACGTGGCTTCGGGGACTCTGTGGAAGAGGCCCTA | 182 |
| (1 bp- | Nsp2, | TCGGAGGCACGTGAACATCTTAAAAGTGGCACTTGTGGCATAGTAG | |
| 1580 bp) | Nsp3 | AGCTGGAAAAAGGCGTATTGCCTCAGCTTGAACAGCCCTATGTGTT | |
| | | CATTAAACGATCTGACGCCCAGGGCACTGGTCATGGCCACAAGGT | |
| | | CTGTGAGCTAGTTGCTGAATTGGATGGCGTTCAGTTCGGTCGTAGC | |
| | | GGTATAACACTGGGAGTACTCGTGCCACACGTTGGCGAGACCCCA | |
| | | ATTGCATACCGCACTGTTCTTCTTCGTAAGAATGGTAATAAGGGAGC | |
| | | CGGTGGCCATAGCTTTGGCATCGATCTAAAGTCATATGACTTAGGT | |
| | | GACGAGCTTGGCACTGATCCCATTGAAGATTATGAACAAAACTGGA | |
| | | ACACTAAACATGGCAGTGGTGCCCTTCGTGAACTCACTCGTGAGCT | |
| | | CAATGGAGGAGTAGTTACTCGCTATGTCGACAACAATTTCTGTGGC | |
| | | CCAGATGGCTACCCCCTTGAATGCATTAAAGACCTTCTCGCTCGTG | |
| | | CGGGCAAGTCAATGTGCACTCTTTCTGAACAACTTGATTATCGAG | |
| | | TCGAAGAGAGGTGTCTACTGCTGTCGTGAACATGAGCATGAAATTG | |
| | | CTTGGTTTACCGAACGCTCTGAAAAGAGCTATGAGCACCAGACACC | |
| | | CTTCGAGATCAAGAGTGCCAAGAAATTTGACACTTTCAAAGGGGAA | |
| | | TGCCCAAAGTTTGTATTTCCTCTCAATTCTAGAGTCAAAGTCATTCA | |
| | | ACCACGTGTTGAAAAGAAAAAGACTGAAGGTTTCATGGGGCGTATA | |
| | | CGCTCTGTGTACCCTGTTGCATCCCCTGGGGATTGTAACGATATGC | |
| | | ACTTGTCTACCTTGATGAAATGTAATCATTGTGATGAAGTTTCATGG | |
| | | CAGACGTGCGACTTTCTCAAAGCCACTTGTGAACAATGTGGCACTG | |
| | | AAAACTTAGTCTGTGAAGGACCCACTACATGTGGATACCTACCTACT | |
| | | AATGCTGTACTTAAAATGCCTTGTCCTGCTTGTCAAGATCCAGAGAT | |
| | | TGGACCTGAGCATAGTGTTGCAGACTATCACAACCACTCAAACATT | |
| | | GAAACTCGACTCCGCAAGGGAGGTAGGACTAAATGTTTTGGTGGGT | |
| | | GTGTGTTTGCCTACGTTGGCTGCTATAACAAGCGTGCCTACTGGGT | |
| | | TCCTCGTGCTAGTGCCGATATTGGTGCAAACCATACTGGCATTACT | |
| | | GGAGACAATGTGGAGACTTTAAATGAAGATCTCCTGGAGATACTGC | |
| | | ATCGTGAACGTGTTAATGTTAACATTGTTGGCGATTTTCAGTTGAAT | |
| | | GAAGAGGTTGCTATTATTCTAGCATCTTTCTCTGCTTCTACTAGTGC | |
| | | CTTTATTGACACTGTAAAGGGCCTTGACTACAAGACCTTCAAAGCCA | |
| | | TTGTTGAATCCTGTGGAAACTACAAAGTTACCAAAGGAAAACCTGTC | |
| | | CAAGGAGCTTGGAACATTGGCCAGCAAAAATCTATTTTGACACCGC | |
| | | TGTGTGGTTTTCCATCACAGGCTGCCAGTGTCATTAGATCAATCTTT | |
| | | TCTCGCAC | |
| 2 | Nsp5_{,} | AAAATTAAGGCTTGCATCGAAGAGGTCACTACAACACTGGAAGAGA | 183 |
| (2547 bp- | Nsp6, | CTAAGTTTCTTACCAATAAGTTGCTTCTTTTTGCTGATATCAGCGGTA | |
| 12830 bp) | Nsp7, | AACTTTACCAAGATTCTCAGAATATGCTTAGAGGTGAGGACGTGTCT | |
| | Nsp8, | TTCCTTGAGAGAGATGCGCCTTACATGGTAGGTGATGTTATCAATA | |
| | Nsp9, | GTGGTGATATTACCTGCGTTGTAATACCTTCTAAGAAGGCTGGTGG | |
| | Nsp10, | TACTACAGAAATGCTTGCAAGAGCATTGAAGAAAGTGCCARTTGAT | |
| | Nsp11, | GAGTATATAACCACATAYCCTGGWCAAGGWTGTGCTGGTTATACAC | |
| | Nsp12, | TTGAKGAAGCTARGACTGCTCTTAARAARTGCAAATCTGCAYTKTAY | |
| | Nsp13, | GTKTTACCTTCAGAATCACCTAATGCTAAGGAAGAGATTCTAGGAAC | |
| | Nsp14, | CGTATCTTGGAATTTGAGAGAAATGCTTGCTCACGCTGAAGAGACA | |
| | Nsp15, | AGAAAATTAATGCCTATCTGCATGGATGTCAGAGCCATAATGGCCA | |
| | Nsp16 | CCATCCAACGCAAGTACAAAGGAATTAAAATTCAAGAAGGCATCGTT | |
| | | GACTATGGTGTCCGATTCTTCTTTTATACTAGTAAAGAGCCTGTAGC | |
| | | TTCTATTATTACGAAGCTGAACTCTCTAAATGAGCCACTTGTCACAA | |
| | | TGCCAATTGGTTATGTGACACATGGTTTTAATCTTGAAGAGGCTGCG | |
| | | CGCTGTATGCGTTCTCTTAAAGCTCCTGCCGTAGTGTCAGTATCATC | |
| | | ACCAGATGCYGTTACTACATATAATGGATACCTCACTTCGTCATCAA | |
| | | AGACATCTGAGGAGCACTTTGTGGAAACAGTTTCTTTGGCTGGCTC | |
| | | TTACAGAGATTGGTCCTATTCAGGACAGCGTACAGAGTTAGGTGTT | |
| | | GAATTTCTTAAGCGTGGTGACAAAATTGTGTACCACACTTTGGAGAG | |
| | | CCCCGTCAAGTTCCATCTTGACGGTGAGGTTCTTCCACTTGACAAA | |
| | | TTAAAGAGTCTCTTATCCCTACGGGAGGTTAAGACTATAAAAGTGTT | |
| | | CACAACTGTGGACAATACTAATCTCCACACACATCTTGTGGATATGT | |
| | | CTATGACATATGGACAGCAGTTTGGTCCAACATATTTGGATGGTGCT | |
| | | GATGTTACAAAAATTAAACCTCATGTAAATCATGAGGGTAAGACTTT | |
| | | CTTTGTATTACCTAGTGATGACACACTACGTAGTGAAGCTTTTGAGT | |
| | | ACTACCACGCTCTTGATGAGAGTTTCCTTGGTAGATACATGTCTGCT | |
| | | TTAAACCACACAAAGAAATGGAAATTCCCTCAAGTTGGTGGTTTGAC | |
| | | TTCCATTAAGTGGGCTGATAACAATTGTTATTTGTCTAGTGTTTTATT | |
| | | AGCACTTCAACAAATTGAAGTTAAATTTAATGCCCCAGCACTACAAG | |
| | | AAGCTTACTATAGAGCTCGTGCTGGTGATGCTGCTAATTTTTGTGCA | |
| | | CTTATACTCGCTTACAGTAATAAAACTGTTGGCGAGCTGGGTGATGT | |
| | | CAGAGAAACTATGGCCCATCTTTTACAGCATGCTAATTTGGAATCTG | |
| | | CAAAGCGAGTTCTTAATGTGGTGTGTAAACATTGCGGCCAGAAAAC | |
| | | TACTACCTTAACGGGTGTAGAGGCTGTGATGTACATGGGTACTCTG | |
| | | TCTTATGATAATCTTAAGACAGGTGTTTCTGTTCCATGTGTGTGTGG | |
| | | TCGTGACGCTACACAATATTTAGTACAACAAGAGTCTTCTTTTGTTAT | |
| | | GATGTCCGCACCACCTGCTGAATATAAATTACAGCAAGGTACATTCT | |
| | | TATGTGCAAATGAATACACTGGTAATTATCAGTGTGGTCATTACACT | |
| | | CATATAACTGCTAAGGAGACCCTCTATCGTATTGATGGAGCTCACCT | |
| | | TACAAAGATGTCAGAGTATAAAGGGCCAGTGACTGATGTGTTCTAC | |
| | | AAGGAAACATCTTACACTACAACCATCAAGCCTGTGTCATATAAACT | |
| | | CGATGGAGTTACTTACACAGAGATTGAACCAAAATTGGATGGGTATT | |
| | | ATAAAAAGGATAATGCTTACTATACGGAGCAGCCTATAGACCTTGTA | |
| | | CCAACTCAACCACTACCAAATGCGAGTTTTGATAATTTCAAACTCAC | |
| | | ATGTTCTAATATAAAATTCGCTGATGATTTAAATCAAATGACAGGCTT | |
| | | CACAAAGCCAGCTTCACGAGAGCTATCTGTCACATTCTTTCCAGACT | |
| | | TGAATGGCGATGTAGTGGCTATTGACTATAGACACTACTCAGCGAG | |
| | | TTTCAAGAAAGGTGCTAGATTACTGCATAAGCCAATTGTTTGGCATA | |
| | | TCAATCAGGCTACAACCAAGACAACGTTCAGACCAAACACTTGGTG | |
| | | TTTACGTTGTCTTTGGAGTACAAAACCAGTAGATACTTCAAATTCATT | |
| | | TGAAGTTCTGGCAGTAGAAGACACACAAGGAATGGACAATCTTGCT | |
| | | TGTGAAAGTCAAAGACCCACCTCTGAAGAAGTAGTGGAAAATCCTA | |
| | | CCATACAGAAGGAAGTCATAGAGTGTGACGTGAAAACTACCGAAGT | |
| | | TGTAGGCAATGTCATACTTAAACCATCAGATGAAGGTGTTAAAGTAA | |
| | | CACAAGAGTTAGGGCATGAGGATCTTATGGCTGCCTATGTGGAAAA | |
| | | TACAAGCATTACCATTAAGAAACCTAATGAGCTTTCATTAGCCTTAG | |
| | | GTTTAAAAACAATTGCCACTCATGGTATTGCTGCAATTAACAGTGTT | |
| | | CCGTGGAGTAAAATTTTGGCTTATGTCAGACCATTCCTAGGACGAA | |
| | | CAGCAATCACAACATCAAACTGTGCTAAGAGATTAGTACAGCGTGT | |
| | | ATTTAACAACTACATGCCCTATGTGCTTACATTATTGTTCCAATTGTG | |
| | | TAGTTTTACCAAAAGTACAAATTCTAGAATTAGAGCTTCACTACCTAC | |
| | | GACTATTGCTAAAAATAGTGTTAAGGGTGTTGCTAAATTATGTTTGG | |
| | | ATGCTGGCATCAATTATGTAAAGTCACCCAAATTTTCTAAATTGTTCA | |
| | | CTATTGCAATGTGGCTATTATTGTTAAGCATTTGCTTAGGTTCACTAA | |
| | | TCTATGTAACTGCAGCTTTAGGTGTATTATTGTCCAACTTTGGAGCT | |
| | | CCTTCCTATTGTAGTGGCGTTAGAGAATCGTATCTCAATTCCTCTAA | |
| | | TGTTACTACTATGGACTTCTGTGAAGGTTCTTTTCCTTGCAGCGTTT | |
| | | GTTTAAGTGGATTAGACTCGCTTGATTCCTATCCAGCTCTTGAAACC | |
| | | ATACAGGTAACGATTTCATCGTATAAGCTAGACTTGACAATTTTAGG | |
| | | TCTGGCTGCTGAGTGGTTTTTGGCATATATGTTGTTCACAAAATTCT | |
| | | TTTATTTATTAGGTCTTTCAGCTATAATGCAGGTGTTCTTTGGCTATT | |
| | | TTGCTAGTCATTTCATCAGCAATTCTTGGCTTATGTGGTTTATCATTA | |
| | | GTATCGTACAAATGGCACCCGTTTCCGCAATGGTTAGGATGTACAT | |
| | | TTTCGTTGCTTCTTTCTACTACATATGGAAGAGCTATGTTCATATTAT | |
| | | GGATGGTTGTACTTCATCTACTTGCATGATGTGCT | |
| 4 | spike | TACCAAGCTACTAGAGTAGTGGTACTTTCATTTGAGCTTCTAAATGC | 184 |
| (22584 bp- | glyco- | ACCTGCCACAGTGTGTGGACCAAAATTGTCCACATCACTAATTAAGA | |
| 24683 bp) | protein | ACCAGTGTGTCAATTTTAATTTCAATGGACTCAAGGGTACTGGTGTG | |
| | | TTGACTGACTCGTCCAAAAAGTTTCAGTCTTTTCAACAATTTGGAAG | |
| | | GGATGCATCTGATTTTACTGACTCAGTACGCGACCCTCAGACACTT | |
| | | CAAATACTTGACATTTCACCATGTTCATTTGGTGGTGTGAGTGTAAT | |
| | | AACACCAGGAACAAATGCTTCATCTGAAGTAGCCGTTCTATACCAA | |
| | | GATGTAAACTGCACTGATGTTCCCACGGCCATACGTGCTGACCAAC | |
| | | TCACACCTGCTTGGCGTGTTTACTCTGCTGGAGTAAATGTGTTTCAA | |
| | | ACTCAGGCTGGCTGTTTAATAGGAGCGGAACATGTCAATGCTTCAT | |
| | | ATGAGTGTGACATTCCCATTGGTGCAGGCATTTGTGCTAGTTACCAT | |
| | | ACAGCTTCCCTTTTACGTAATACAGGCCAGAAATCAATTGTGGCCTA | |
| | | TACTATGTCACTTGGTGCTGAAAACTCAATTGCTTATGCTAATAACT | |
| | | CAATTGCCATACCTACAAATTTTTCAATCAGTGTCACAACTGAAGTG | |
| | | ATGCCTGTTTCAATGGCTAAGACATCAGTAGATTGTACAATGTACAT | |
| | | CTGTGGTGACTCTCAGGAGTGCAGCAACTTACTACTTCAGTATGGT | |
| | | AGCTTTTGCACACAATTAAATCGTGCCCTTTCAGGCATTGCTGTTGA | |
| | | ACAGGACAAAAACACTCAAGAGGTTTTTGCCCAAGTTAAACAAATGT | |
| | | ATAAGACACCAGCCATAAAAGATTTTGGTGGCTTTAATTTCTCACAA | |
| | | ATATTGCCTGACCCTTCTAAGCCAACAAAAAGATCATTTATTGAGGA | |
| | | TTTACTCTTCAACAAAGTGACTCTCGCTGATGCTGGCTTTATGAAGC | |
| | | AATACGGCGAATGCCTAGGCGATATTAGTGCTAGAGATCTCATTTG | |
| | | TGCGCAGAAGTTCAATGGACTCACTGTCCTTCCACCTCTACTCACG | |
| | | GATGAAATGATTGCTGCTTACACCGCCGCTCTTGTCAGCGGTACTG | |
| | | CTACTGCTGGTTGGACATTTGGTGCAGGTGCTGCTCTACAAATACC | |
| | | TTTTGCTATGCAAATGGCTTATAGGTTCAATGGCATTGGAGTTACTC | |
| | | AAAATGTTCTCTATGAGAACCAGAAGCAGATCGCTAACCAATTTAAC | |
| | | AAGGCGATCAGTCAAATTCAAGAATCACTTACTACTACTTCAACTGC | |
| | | ATTGGGCAAGCTGCAAGACGTCGTCAACCAGAATGCTCAAGCATTG | |
| | | AACACACTTGTTAAACAACTAAGTTCTAACTTTGGTGCAATTTCAAGT | |
| | | GTTTTAAATGACATTCTGTCTCGACTYGACAAAGTTGAGGCTGAAGT | |
| | | GCAAATTGATAGGTTGATTACTGGCAGATTACAAAGCCTTCAGACCT | |
| | | ATGTAACACAACAACTAATCAGAGCTGCTGAAATCAGAGCTTCTGC | |
| | | CAATCTTGCTGCCACTAAGATGTCCGAGTGTGTTCTTGGACAATCAA | |
| | | AAAGAGTTGACTTTTGTGGAAAAGGCTATCATCTTATGTCTTTCCCT | |
| | | CAAGCAGCCCCACATGGTGTCGTCTTCTTACATGTCACATACGTGC | |
| | | CATCGCAAGAAAGAAACTTCACCACTGCCCCAGCAATCTGCCATCA | |
| | | AGGCAAGGCACACTTCCCTCGTGAAGGTGTTTTTGTATCTAATGGC | |
| | | ACTTCTTGGTTTATCACACAGAGGAACTTCTTCACCACAAATAATT | |
| | | ACAACAGACAATACATTTGTCTCTGGAAATTGTGATGTCGTTATTGG | |
| | | CATCATCAACAATACTGTTTATGATCCTCTGCAACCTGAGCTTGACT | |
| | | CATTTAAAGAAGAGCTGGACAAGTACTTCAAAAACCACACGTCACCT | |
| | | GATGTRGATCTTGGCGACATCTCAGGCATTAATGCTTCAGTCGTCA | |
| | | ATATTCAAAAAGAAATTGACCGCCTCAATGAGGTTGCCAAAAATCTA | |
| | | AATGAATCGCTCATCGATCTTCAAGAACTTGGAAAATATGAGCA | |
| 5 | ORF3a, | CAGTAACACTTGCTTGCTTTGTGCTTGCTGCTGTTTACAGAATTAAT | 185 |
| (26193 bp- | Envelope | TGGGTGACTGGCGGAATTGCRATTGCAATGGCTTGTATTGTAGGCT | |
| 27421 bp) | (E), | TGATGTGGCTTAGCTACTTCRTTGCTTCTTTCAGGCTGTTTGCGCGC | |
| | Membrane | ACCCGCTCWATGTGGTCATTCAACCCAGAAACYAACATTCTTCTCA | |
| | (M), ORF6, | ATGTGCCTCTTCGRGGRACAATYTTGACCAGACCGCTCATGGARAG | |
| | ORF7a | TGAACTTGTCATTGGTGCTGTGATCATTCGTGGTCACCTGCGAATG | |
| | | GCTGGACACTCYCTWGGGCGCTGTGACATTAAGGACCTGCCAAAA | |
| | | GAGATCACTGTGGCTACATCACGAACGCTTTCTTATTACAAATTAGG | |
| | | AGCTTCGCAGCGTGTAGGCACTGACTCAGGTTTTGCTGCATACAAC | |
| | | CGCTACCGTATTGGAAACTACAAATTAAATACAGACCACGCCGGTA | |
| | | GCAACGACAATATTGCTTTGCTAGTACAGTAAGTGACAACAGATGTT | |
| | | TCATCTAGTTGACTTCCAGGTTACAATAGCGGAGATATTGATTATCA | |
| | | TTATGAGGACTTTCAGGATTGCCATCTGGAATCTTGATGTAATAATA | |
| | | AGTTCAATAGTGAGACAATTATTTAAGCCTCTAACTAAGAAGAATTAT | |
| | | TCTGAGTTAGATGATGAAGAACYTATGGAGATTGATTATCCATAAAA | |
| | | CGAACATGAAAATTATCCTCTTCCTGACTTTGATTTCACTTGCATTTT | |
| | | GTGAGTTATATCATTATCAGGAGTGTGTTAGAGGTACAACTGTACTA | |
| | | TTAAAAGAACCTTGCCCATCRGGAACGTACGAGGGCAATTCACCAT | |
| | | TTCACCCTCTTGCTGACAACAAATTTGCACTAACTTGCATTAGCACA | |
| | | CATTTTGCTTTTGCTTGTGCTGACGGTACTCGACATACCTATCAGCT | |
| | | TCGTGCAAGATCAGTTTCTCCAAAACTCTTCATCAGGCAAGAGGAAT | |
| | | TTCATCAAGAGCTCTATTCACCACTTTTTCTCATTGTTGCCGCTCTA | |
| | | GTATTTATAATACTTTGCTTCACCATTAAGAGAAAGACCGAATGAGT | |
| | | GAGCTCACTTTAATTGACTTCTATTTGTGCTTTAGCCTTTCTGCTA | |
| | | TTCCTTGTTTTAATAATGCTCATCATATTTTGGTTCTCCTTGGAGATT | |
| | | CAAGATTCTGAAGAGCCATGTCCAAAAGTCTAAACGAACATGAAACT | |
| | | TCTCATTGTTTT | |
| 6 | Nsp2 | ATGGCATACACTCGCTATGTCGATAACAACTTCTGTGGCCCTGATG | 360 |
| | | GCTACCCTCTTGAGTGCATTAAAGACCTTCTAGCACGTGCTGGTAA | |
| | | AGCTTCATGCACTTTGTCCGAACAACTGGACTTTATTGACACTAAGA | |
| | | GGGGTGTATACTGCTGCCGTGAACATGAGCATGAAATTGCTTGGTA | |
| | | CACGGAACGTTCTGAAAAGAGCTATGAATTGCAGACACCTTTGAA | |
| | | ATTAAATTGGCAAAGAAATTTGACACCTTCAATGGGGAATGTCCAAA | |
| | | TTTTGTATTTCCCTTAAATTCCATAATCAAGACTATTCAACCAAGGGT | |
| | | TGAAAAGAAAAAGCTTGATGGCTTTATGGGTAGAATTCGATCTGTCT | |
| | | ATCCAGTTGCGTCACCAAATGAATGCAACCAAATGTGCCTTTCAACT | |
| | | CTCATGAAGTGTGATCATTGTGGTGAAACTTCATGGCAGACGGGCG | |
| | | ATTTTGTTAAAGCCACTTGCGAATTTTGTGGCACTGAGAATTTGACT | |
| | | AAAGAAGGTGCCACTACTTGTGGTTACTTACCCCAAAATGCTGTTGT | |
| | | TAAAATTTATTGTCCAGCATGTCACAATTCAGAAGTAGGACCTGAGC | |
| | | ATAGTCTTGCCGAATACCATAATGAATCTGGCTTGAAAACCATTCTT | |
| | | CGTAAGGGTGGTCGCACTATTGCCTTTGGAGGCTGTGTGTTCTCTT | |
| | | ATGTTGGTTGCCATAACAAGTGTGCCTATTGGGTTCCACGTGCTAG | |
| | | CGCTAACATAGGTTGTAACCATACAGGTGTTGTTGGAGAAGGTTCC | |
| | | GAAGGTCTTAATGACAACCTTCTTGAAATACTCCAAAAAGAGAAAGT | |
| | | CAACATCAATATTGTTGGTGACTTTAAACTTAATGAAGAGATCGCCA | |
| | | TTATTTTGGCATCTTTTTCTGCTTCCACAAGTGCTTTTGTGGAAACTG | |
| | | TGAAAGGTTTGGATTATAAAGCATTCAAACAAATTGTTGAATCCTGT | |
| | | GGTAATTTTAAAGTTACAAAAGGAAAAGCTAAAAAAGGTGCCTGGAA | |
| | | TATTGGTGAACAGAAATCAATACTGAGTCCTCTTTATGCATTTGCAT | |
| | | CAGAGGCTGCTCGTGTTGTACGATCAATTTTCTCCCGCACTCTTGA | |
| | | AACTGCTCAAAATTCTGTGCGTGTTTTACAGAAGGCCGCTATAACAA | |
| | | TACTAGATGGAATTTCACAGTATTCACTGAGACTCATTGATGCTATG | |
| | | ATGTTCACATCTGATTTGGCTACTAACAATCTAGTTGTAATGGCCTA | |
| | | CATTACAGGTGGTGTTGTTCAGTTGACTTCGCAGTGGCTAACTAAC | |
| | | ATCTTTGGCACTGTTTATGAAAAACTCAAACCCGTCCTTGATTGGCT | |
| | | TGAAGAGAAGTTTAAGGAAGGTGTAGAGTTTCTTAGAGACGGTTGG | |
| | | GAAATTGTTAAATTTATCTCAACCTGTGCTTGTGAAATTGTCGGTGG | |
| | | ACAAATTGTCACCTGTGCAAAGGAAATTAAGGAGAGTGTTCAGACA | |
| | | TTCTTTAAGCTTGTAAATAAATTTTTGGCTTTGTGTGCTGACTCTATC | |
| | | ATTATTGGTGGAGCTAAACTTAAAGCCTTGAATTTAGGTGAAACATT | |
| | | TGTCACGCACTCAAAGGGATTGTACAGAAAGTGTGTTAAATCCAGA | |
| | | GAAGAAACTGGCCTACTCATGCCTCTAAAAGCCCCAAAAGAAATTAT | |
| | | CTTCTTAGAGGGAGAAACACTTCCCACAGAAGTGTTAACAGAGGAA | |
| | | GTTGTCTTGAAAACTGGTGATTTACAACCATTAGAACAACCTACTAG | |
| | | TGAAGCTGTTGAAGCTCCATTGGTTGGTACACCAGTTTGTATTAACG | |
| | | GGCTTATGTTGCTCGAAATCAAAGACACAGAAAAGTACTGTGCCCT | |
| | | TGCACCTAATATGATGGTAACAAACAATACCTTCACACTCAAAGGCG | |
| | | GTTGATGA | |
| 7 | Nsp2 | ATGGCCTATACCAGGTACGTGGATAATAATTTCTGTGGGCCTGACG | 361 |
| | (codon | GCTACCCTCTGGAATGCATCAAAGACCTGTTGGCTAGGGCAGGAAA | |
| | optimized) | AGCTTCATGCACCCTGTCTGAGCAACTTGACTTCATTGACACAAAG | |
| | | CGGGGAGTCTACTGCTGCCGCGAACATGAGCACGAGATTGCTTGG | |
| | | TACACAGAGCGAAGCGAAAAATCTTATGAACTGCAGACACCCTTTG | |
| | | AAATCAAGTTAGCAAAGAAGTTCGACACCTTTAATGGCGAGTGCCC | |
| | | AAACTTTGTGTTCCCTCTGAATTCGATCATTAAAACCATCCAGCCTA | |
| | | GAGTAGAAAAAAAAAAGCTCGATGGTTTCATGGGCCGCATAAGATC | |
| | | TGTGTATCCTGTGGCTAGCCCCAACGAGTGTAACCAGATGTGTTTG | |
| | | TCAACCCTGATGAAATGTGATCACTGTGGAGAAACGAGTTGGCAGA | |
| | | CTGGAGACTTTGTTAAAGCTACTTGCGAATTCTGCGGGACAGAGAA | |
| | | CCTCACCAAGGAGGGAGCAACCACGTGCGGTTACCTGCCCCAAAA | |
| | | TGCTGTAGTCAAAATCTACTGTCCGGCATGCCATAACTCAGAGGTT | |
| | | GGGCCAGAGCATAGCTTGGCAGAGTACCATAATGAATCGGGACTTA | |
| | | AAACAATCCTGCGGAAGGGTGGCCGTACGATTGCCTTTGGAGGGT | |
| | | GCGTCAGCTATGTGGGCTGCCACAACAAGTGCGCCTACTGGGT | |
| | | GCCCAGAGCCAGTGCCAACATTGGATGTAACCACACAGGCGTCGT | |
| | | TGGGGAGGGCAGTGAGGGCTTGAATGACAATCTTCTGGAGATTCTA | |
| | | CAAAAGGAAAAGGTCAACATCAACATAGTTGGGGATTTCAAGCTGA | |
| | | ACGAGGAGATTGCCATCATTTTAGCGTCCTTTTCTGCCTCAACAAGT | |
| | | GCCTTCGTGGAAACTGTGAAAGGGCTTGACTACAAGGCATTTAAGC | |
| | | AGATCGTGGAGTCCTGTGGCAACTTTAAAGTGACTAAGGGCAAGGC | |
| | | CAAAAAGGGGGCCTGGAATATAGGCGAACAGAAGTCCATCCTGAG | |
| | | CCCTCTCTATGCTTTTGCTAGTGAAGCTGCCCGCGTTGTCCGGTCT | |
| | | ATCTTCAGCCGAACTTTGGAGACTGCTCAGAACTCTGTCAGAGTCC | |
| | | TGCAGAAGGCAGCCATTACCATACTTGATGGGATATCTCAATACAG | |
| | | CCTCCGTCTGATTGATGCCATGATGTTTACTTCAGATCTCGCAACCA | |
| | | ACAATCTCGTTGTGATGGCTTATATCACTGGGGGTGTGGTACAGCT | |
| | | GACGTCCCAGTGGCTGACTAATATATTCGGGACCGTATATGAGAAG | |
| | | CTCAAGCCAGTGCTGGACTGGCTGGAAGAGAAATTCAAGGAAGGT | |
| | | GTGGAATTTTTGAGGGACGGCTGGGAGATTGTGAAGTTTATTAGCA | |
| | | CCTGTGCATGTGAGATTGTTGGCGGACAGATCGTGACATGTGCTAA | |
| | | GGAAATAAAAGAGAGTGTACAAACGTTCTTCAAACTCGTGAATAAAT | |
| | | TTTTAGCGCTATGTGCTGACTCCATCATAATTGGCGGAGCCAAGTTA | |
| | | AAAGCGCTCAACTTGGGAGAGACATTTGTCACCCACTCCAAAGGTC | |
| | | TGTATAGGAAGTGTGTAAAGTCCAGAGAGGAGACTGGACTACTAAT | |
| | | GCCACTGAAGGCTCCCAAAGAGATCATCTTCTTAGAAGGAGAGACA | |
| | | CTTCCGACTGAGGTCCTTACAGAAGAAGTGGTCCTCAAAACTGGTG | |
| | | ATCTCCAGCCCCTAGAGCAGCCCACATCCGAGGCAGTCGAAGCCC | |
| | | CACTGGTGGGCACACCTGTGTGCATCAATGGACTCATGTTACTCGA | |
| | | AATCAAGGATACCGAGAAGTACTGCGCACTGGCCCCTAACATGATG | |
| | | GTAACCAATAACACATTCACCCTGAAAGGTGGTTGATAA | |
| 8 | Nsp3 | ATGCTCTTGTTAGTGACATTGACATCACTTTCTTAAAGAAAGATGCT | 362 |
| | | CCATATATAGTGGGTGATGTTGTTCAAGAGGGTGTTTTAACTGCTGT | |
| | | GGTTATACCTACTAAAAAGGCTAGTGGCACTACTGAAATGCTAGCG | |
| | | AAAGCTTTGAGAAAAGTGCCAACAGACAATTATATAACCACTTACCC | |
| | | GGGTCAGGGTTTAAATGGTTACACTGTAGAGGAGGCAAAGACAGTG | |
| | | CTTAAAAAGTGTAAAAGTGCTTTTTACATTCTACCATCTATTATCTCT | |
| | | AATGAGAAGCAAGAAATTCTTGGAACTGTTTCTTGGAATTTGCGAGA | |
| | | AATGCTTGCACATGCAGAAGAAACACGCAAATTAATGCCTGTCTGT | |
| | | GTGGAAACTAAAGCCATAGTTTCAACTATACAGCGTAAATATAAGGG | |
| | | TATTAAAATACAAGAGGGTGTGGTTGATTATGGTGCTAGATTTTACT | |
| | | TTTACACCAGTAAAACAACTGTAGCGTCACTTATCAACACACTTAAC | |
| | | GATCTAAATGAAACTCTTGTTACAATGCCACTTGGCTATGTAACACA | |
| | | TGGCTTAAATTTGGAAGAAGCTGCTCGGTATATGAGATCTCTCAAAG | |
| | | TGCCAGCTACAGTTTCTGTTTCTTCACCTGATGCTGTTACAGCGTAT | |
| | | AATGGTTATCTTACTTCTTCTTCTAAAACACCTGAAGAACATATT | |
| | | GAAACCATCTCACTTGCTGGTTCCTATAAAGATTGGTCCTATTCTGG | |
| | | ACAATCTACACAACTAGGTATAGAATTTCTTAAGAGAGGTGATAAAA | |
| | | GTGTATATTACACTAGTAATCCTACCACATTCCACCTAGATGGTGAA | |
| | | GTTATCACCTTTGACAATCTTAAGACACTTCTTTCTTTGAGAGAAGT | |
| | | GAGGACTATTAAGGTGTTTACAACAGTAGACAACATTAACCTCCACA | |
| | | CGCAAGTTGTGGACATGTCAATGACATATGGACAACAGTTTGGTCC | |
| | | AACTTATTTGGATGGAGCTGATGTTACTAAAATAAAACCTCATAATTC | |
| | | ACATGAAGGTAAAACATTTTATGTTTTACCTAATGATGACACTCTAC | |
| | | GTGTTGAGGCTTTTGAGTACTACCACACAACTGATCCTAGTTTTCTG | |
| | | GGTAGGTACATGTCAGCATTAAATCACACTAAAAAGTGGAAATACCC | |
| | | ACAAGTTAATGGTTTAACTTCTATTAAATGGGCAGATAACAACTGTT | |
| | | ATCTTGCCACTGCATTGTTAACACTCCAACAAATAGAGTTGAAGTTT | |
| | | AATCCACCTGCTCTACAAGATGCTTATTACAGAGCAAGGGCTGGTG | |
| | | AAGCTGCTAACTTTTGTGCACTTATCTTAGCCTACTGTAATAAGACA | |
| | | GTAGGTGAGTTAGGTGATGTTAGAGAAACAATGAGTTACTTGTTTCA | |
| | | ACATGCCAATTTAGATTCTTGCAAAAGAGTCTTGAACGTGGTGTGTA | |
| | | AAACTTGTGGACAACAGCAGACAACCCTTAAGGGTGTAGAAGCTGT | |
| | | TATGTACATGGGCACACTTTCTTATGAACAATTTAAGAAAGGTGTTC | |
| | | AGATACCTTGTACGTGTGGTAAACAAGCTACAAAATATCTAGTACAA | |
| | | CAGGAGTCACCTTGTTATGATGTCAGCACCACCTGCTCAGTATG | |
| | | AACTTAAGCATGGTACATTTACTTGTGCTAGTGAGTACACTGGTAAT | |
| | | TACCAGTGTGGTCACTATAAACATATAACTTCTAAAGAAACTTTGTAT | |
| | | TGCATAGACGGTGCTTTACTTACAAAGTCCTCAGAATACAAAGGTCC | |
| | | TATTACGGATGTCTACAAAGAAAACAGTTACACAACAACCATAA | |
| | | AACCAGTTACTTATAAATTGGATGGTGTTGTTTGTACAGAAATTGAC | |
| | | CCTAAGTTGGACAATTATTATAAGAAAGACAATTCTTATTTCACAGA | |
| | | GCAACCAATTGATCTTGTACCAAACCAACCATATCCAAACGCAAGCT | |
| | | TCGATAATTTTAAGTTTGTATGTGATAATATCAAATTTGCTGATGATT | |
| | | TAAACCAGTTAACTGGTTATAAGAAACCTGCTTCAAGAGAGCTTAAA | |
| | | GTTACATTTTTCCCTGACTTAAATGGTGATGTGGTGGCTATTGATTA | |
| | | TAAACACTACACACCCTCTTTTAAGAAAGGAGCTAAATTGTTACATA | |
| | | AACCTATTGTTTGGCATGTTAACAATGCAACTAATAAAGCCACGTAT | |
| | | AAACCAAATACCTGGTGTATACGTTGTCTTTGGAGCACAAAACCAGT | |
| | | TGAAACATCAAATTCGTTTGATGTACTGAAGTCAGAGGACGCGCAG | |
| | | GGAATGGATAATCTTGCCTGCGAAGATCTAAAACCAGTCTCTGAAG | |
| | | AAGTAGTGGAAAATCCTACCATACAGAAAGACGTTCTTGAGTGTAAT | |
| | | GTGAAAACTACCGAAGTTGTAGGAGACATTATACTTAAACCAGCAAA | |
| | | TAATAGTTTAAAAATTACAGAAGAGGTTGGCCACACAGATCTAATGG | |
| | | CTGCTTATGTAGACAATTCTAGTCTTACTATTAAGAAACCTAATGAAT | |
| | | TATCTAGAGTATTAGGTTTGAAAACCCTTGCTACTCATGGTTTAGCT | |
| | | GCTGTTAATAGTGTCCCTTGGGATACTATAGCTAATTATGCTAAGCC | |
| | | TTTTCTTAACAAAGTTGTTAGTACAACTACTAACATAGTTACACGGTG | |
| | | TTTAAACCGTGTTTGTACTAATTATATGCCTTATTTCTTTACTTTATTG | |
| | | CTACAATTGTGTACTTTTACTAGAAGTACAAATTCTAGAATTAAAGCA | |
| | | TCTATGCCGACTACTATAGCAAAGAATACTGTTAAGAGTGTCGGTAA | |
| | | ATTTTGTCTAGAGGCTTCATTTAATTATTTGAAGTCACCTAATTTTTC | |
| | | TAAACTGATAAATATTATAATTTGGTTTTTACTATTAAGTGTTTGCCTA | |
| | | GGTTCTTTAATCTACTCAACCGCTGCTTTAGGTGTTTTAATGTCTAAT | |
| | | TTAGGCATGCCTTCTTACTGTACTGGTTACAGAGAAGGCTATTTGAA | |
| | | CTCTACTAATGTCACTATTGCAACCTACTGTACTGGTTCTATACCTT | |
| | | GTAGTGTTTGTCTTAGTGGTTTAGATTCTTTAGACACCTATCCTTCTT | |
| | | TAGAAACTATACAAATTACCATTTCATCTTTTAAATGGGATTTAACTG | |
| | | CTTTTGGCTTAGTTGCAGAGTGGTTTTTGGCATATATTCTTTTCACTA | |
| | | GGTTTTTCTATGTACTTGGATTGGCTGCAATCATGCAATTGTTTTTCA | |
| | | GCTATTTTGCAGTACATTTTATTAGTAATTCTTGGCTTATGTGGTTAA | |
| | | TAATTAATCTTGTACAAATGGCCCCGATTTCAGCTATGGTTAGAATG | |
| | | TACATCTTCTTTGCATCATTTTATTATGTATGGAAAAGTTATGTGCAT | |
| | | GTTGTAGACGGTTGTAATTCATCAACTTGTATGATGTGTTACAAACG | |
| | | TAATAGAGCAACAAGAGTCGAATGTACAACTATTGTTAATGGTGTTA | |
| | | GAAGGTCCTTTTATGTCTATGCTAATGGAGGTAAAGGCTTTTGCAAA | |
| | | CTACACAATTGGAATTGTGTTAATTGTGATACATTCTGTGCTGGTAG | |
| | | TACATTTATTAGTGATGAAGTTGCGAGAGACTTGTCACTACAGTTTA | |
| | | AAAGACCAATAAATCCTACTGACCAGTCTTCTTACATCGTTGATAGT | |
| | | GTTACAGTGAAGAATGGTTCCATCCATCTTTACTTTGATAAAGCTGG | |
| | | TCAAAAGACTTATGAAAGACATTCTCTCTCTCATTTTGTTAACTTAGA | |
| | | CAACCTGAGAGCTAATAACACTAAAGGTTCATTGCCTATTAATGTTA | |
| | | TAGTTTTTGATGGTAAATCAAAATGTGAAGAATCATCTGCAAAATCA | |
| | | GCGTCTGTTTACTACAGTCAGCTTATGTGTCAACCTATACTGTTACT | |
| | | AGATCAGGCATTAGTGTCTGATGTTGGTGATAGTGCGGAAGTTGCA | |
| | | GTTAAAATGTTTGATGCTTACGTTAATACGTTTTCATCAACTTTTAAC | |
| | | GTACCAATGGAAAAACTCAAAACACTAGTTGCAACTGCAGAAGCTG | |
| | | AACTTGCAAAGAATGTGTCCTTAGACAATGTCTTATCTACTTTTATTT | |
| | | CAGCAGCTCGGCAAGGGTTTGTTGATTCAGATGTAGAAACTAAAGA | |
| | | TGTTGTTGAATGTCTTAAATTGTCACATCAATCTGACATAGAAGTTAC | |
| | | TGGCGATAGTTGTAATAACTATATGCTCACCTATAACAAAGTTGAAA | |
| | | ACATGACACCCCGTGACCTTGGTGCTTGTATTGACTGTAGTGCGCG | |
| | | TCATATTAATGCGCAGGTAGCAAAAAGTCACAACATTGCTTTGATAT | |
| | | GGAACGTTAAAGATTTCATGTCATTGTCTGAACAACTACGAAAACAA | |
| | | ATACGTAGTGCTGCTAAAAAGAATAACTTACCTTTTAAGTTGACATG | |
| | | TGCAACTACTAGACAAGTTGTTAATGTTGTAACAACAAAGATAGCAC | |
| | | TTAAGGGTGGTTGATGA | |
| 9 | Nsp3 | ATGCTCTTACTTGTTATTGACATCACCTTTCTAAAGAAAGATGCCCC | 363 |
| | (codon | TTATATTGTGGGAGACGTTGTCCAGGAGGGAGTCCTTACGGCCGTG | |
| | optimized) | GTGATTCCAACAAAGAAGGCTAGTGGCACCACAGAAATGCTGGCCA | |
| | | AGGCCTTGCGCAAGGTGCCGACAGACAACTACATCACTACATATCC | |
| | | TGGACAAGGCCTGAACGGATATACAGTGGAAGAGGCAAAGACTGT | |
| | | GCTAAAGAAGTGCAAGTCTGCCTTTTACATCCTTCCAAGCATTATTA | |
| | | GCAACGAGAAGCAGGAAATACTCGGAACCGTAAGCTGGAACCTTA | |
| | | GAGAGATGCTTGCGCATGCTGAGGAAACCCGAAAACTGATGCCTGT | |
| | | GTGTGTTGAAACCAAAGCCATCGTTTCTACCATCCAGCGAAAGTATA | |
| | | AGGGTATCAAGATCCAAGAGGGAGTGGTAGACTATGGCGCTAGATT | |
| | | CTACTTCTATACCTCTAAAACAACTGTCGCCTCTCTTATCAATACTCT | |
| | | GAATGACCTGAATGAGACTCTTGTGACCATGCCCCTGGGATACGTA | |
| | | ACCCATGGGCTTAACTTGGAAGAAGCCGCTCGCTACATGCGTAGCC | |
| | | TGAAGGTCCCTGCTACTGTTAGTGTCTCTTCCCCCGATGCCGTCAC | |
| | | AGCTTACAACGGGTATCTGACGTCAAGCTCCAAAACTCCTGAAGAG | |
| | | CACTTCATCGAGACCATTAGTCTAGCAGGATCCTATAAAGACTGGA | |
| | | GTTACTCAGGCCAGAGCACCCAGCTGGGGATAGAGTTCTTGAAAC | |
| | | GTGGAGACAAGTCCGTCTACTACACATCAAATCCCACGACGTTCCA | |
| | | CCTGGATGGGGAAGTGATAACCTTTGATAACTTAAAAACCCTGCTG | |
| | | AGCCTGAGGGAAGTCCGGACTATCAAGGTTTTTACTACAGTGGATA | |
| | | ATATTAACCTTCACACACAGGTGGTGGACATGTCCATGACCTATGG | |
| | | GCAGCAGTTCGGTCCTACCTACCTGGACGGAGCTGACGTTACCAAA | |
| | | ATCAAACCCCACAACTCTCATGAGGGAAAGACGTTCTACGTGCTGC | |
| | | CCAATGATGACACCTTGCGAGTGGAGGCATTTGAGTATTACCACAC | |
| | | TACCGATCCGTCTTTCTTAGGCCGCTATATGTCCGCTTTAAATCATA | |
| | | CAAAGAAGTGGAAGTACCCTCAGGTAAACGGCCTGACATCTATTAA | |
| | | ATGGGCCGACAACAACTGCTATCTGGCAACTGCTTTGCTCACACTT | |
| | | CAGCAGATTGAGTTGAAATTTAACCCTCCCGCACTGCAGGACGCTT | |
| | | ATTATCGGGCAAGGGCCGGTGAGGCGGCTAACTTCTGTGCTCTCAT | |
| | | CCTTGCTTACTGTAACAAAACAGTGGGCGAGCTAGGAGATGTCAGG | |
| | | GAGACAATGTCTTACTTGTTTCAGCACGCCAACTTAGACTCCTGCAA | |
| | | AAGAGTGCTCAATGTAGTCTGCAAAACCTGCGGTCAGCAGCAGACT | |
| | | ACCTTGAAGGGGGTCGAAGCAGTGATGTACATGGGTACACTATCCT | |
| | | ATGAGCAATTTAAAAAGGGTGTTCAGATCCCCTGCACATGTGGCAA | |
| | | GCAGGCAACAAAATACCTCGTGCAGCAAGAATCCCCATTTGTTATG | |
| | | ATGAGCGCACCTCCAGCCCAGTACGAACTGAAACATGGAACATTTA | |
| | | CCTGCGCTTCGGAGTACACTGGCAATTACCAGTGTGGGCATTACAA | |
| | | GCACATCACGTCCAAAGAAACACTCTACTGCATAGATGGAGCCCTG | |
| | | TTGACCAAGTCCAGCGAATATAAAGGCCCTATCACAGATGTTTTCTA | |
| | | CAAGGAGAATTCATATACAACCACCATCAAGCCCGTGACATACAAG | |
| | | TTAGACGGCGTGGTATGTACAGAAATTGACCCCAAGCTGGACAATT | |
| | | ACTACAAAAAAGACAATAGCTATTTTACTGAACAACCAATCGATCTT | |
| | | GTCCCTAATCAGCCCTACCCCAATGCGTCATTTGATAACTTTAAGTT | |
| | | TGTGTGTGATAATATTAAATTTGCAGATGATCTAAACCAGTTGACGG | |
| | | GATACAAGAAACCCGCCTCGCGCGAACTGAAAGTGACTTTTTTTCC | |
| | | AGATCTGAATGGGGATGTCGTGGCCATAGATTATAAGCATTATACTC | |
| | | CAAGTTTCAAGAAAGGCGCTAAGTTATTACATAAGCCTATTGTCTGG | |
| | | CATGTCAACAATGCTACAAATAAAGCCACTTATAAGCCAAACACATG | |
| | | GTGTATTAGGTGCCTGTGGAGCACAAAACCAGTGGAGACTAGCAAT | |
| | | TCCTTTGACGTCCTGAAGAGTGAAGATGCACAAGGCATGGATAACT | |
| | | TGGCCTGTGAGGACCTGAAACCAGTCTCAGAGGAAGTGGTGGAAA | |
| | | ATCCAACCATCCAGAAAGACGTACTGGAGTGTAACGTGAAAACCAC | |
| | | TGAGGTTGTGGGGGACATTATCTTAAAACCTGCTAACAACAGCCTG | |
| | | AAGATTACCGAGGAGGTAGGACACACTGATTTAATGGCAGCTTACG | |
| | | TAGATAACTCCAGTCTGACCATCAAGAAGCCTAACGAGCTGAGTCG | |
| | | GGTGCTAGGCCTCAAAACTCTGGCCACCCACGGCCTAGCGGCCGT | |
| | | TAATTCTGTACCGTGGGATACAATCGCTAATTATGCCAAGCCCTTCC | |
| | | TCAACAAGGTTGTCAGCACGACCACCAACATCGTGACCCGATGCTT | |
| | | GAACCGTGTGTGCACGAACTATATGCCCTATTTCTTTACCCTTCTAC | |
| | | TCCAGCTGTGTACTTTCACCCGGTCTACAAACAGTCGGATCAAGGC | |
| | | CAGCATGCCAACCACAATTGCCAAGAACACCGTTAAATCCGTTGGG | |
| | | AAGTTCTGCCTTGAGGCTAGCTTCAACTACCTGAAATCACCAAACTT | |
| | | CTCGAAGCTGATTAACATTATAATATGGTTCCTGCTCCTGTCCGTGT | |
| | | GTCTTGGAAGCCTTATCTACTCCACAGCTGCCCTGGGCGTCCTCAT | |
| | | GTCTAATCTGGGTATGCCTAGTTACTGCACAGGCTACCGGGAAGGT | |
| | | TATTTGAATAGCACTAACGTTACAATCGCCACATACTGCACAGGGA | |
| | | GCATTCCTTGCTCCGTTTGTTTGTCAGGTTTGGATTCTCTCGATACT | |
| | | TATCCCTCTCTGGAAACTATTCAGATCACGATTTCTAGTTTCAAATG | |
| | | GGACCTCACGGCATTTGGGCTCGTGGCCGAGTGGTTCCTGGCCTA | |
| | | TATACTCTTCACTAGGTTCTTCTATGTCCTGGGGCTGGCTGCCATCA | |
| | | TGCAGCTCTTTTTCAGTTATTTCGCAGTCCATTTTATCAGCAATAGCT | |
| | | GGCTCATGTGGCTCATAATAAATTTAGTACAGATGGCACCAATCTCG | |
| | | GCCATGGTGAGGATGTATATCTTTTTCGCATCCTTTTACTATGTGTG | |
| | | GAAATCATATGTCCACGTGGTGGACGGATGCAATAGTAGCACGTGC | |
| | | ATGATGTGCTACAAACGGAATAGAGCGACCCGCGTGGAATGTACCA | |
| | | CCATTGTGAATGGGGTTCGCAGATCATTCTACGTATATGCCAACGG | |
| | | CGGCAAGGGCTTCTGCAAGCTCCATAACTGGAACTGTGTCAATTGT | |
| | | GACACATTGTGCAGGATCAACTTTCATCAGTGACGAGGTCGCGC | |
| | | GGGACCTCAGTCTGCAATTCAAGAGACCGATCAACCCTACCGACCA | |
| | | GTCATCTTACATCGTGGATTCAGTTACCGTCAAGAATGGATCCATTC | |
| | | ACTTATACTTCGATAAAGCAGGCCAAAAGACTTACGAGAGGCACTC | |
| | | TCTCAGCCACTTTGTGAACCTGGACAATCTGAGGGCAAATAACACC | |
| | | AAGGGGTCTCTTCCCATTAATGTGATAGTGTTTGACGGGAAGAGCA | |
| | | AGTGTGAGGAGAGCAGCGCCAAATCTGCCTCAGTGTACTACAGCC | |
| | | AGCTCATGTGTCAACCAATACTCCTTCTAGACCAAGCCCTGGTCTC | |
| | | AGACGTTGGGGACAGTGCTGAAGTGGCTGTGAAGATGTTCGATGC | |
| | | TTATGTGAACACATTTTCTTCCACTTTCAACGTCCCTATGGAGAAAC | |
| | | TAAAGACGCTGGTGGCAACGGCCGAGGCCGAGCTGGCCAAGAAC | |
| | | GTTTCATTGGACAATGTGCTCTCGACATTCATCTCCGCTGCAAGACA | |
| | | GGGCTTTGTGGACTCAGATGTAGAAACCAAGGATGTGGTCGAGTG | |
| | | CTTGAAGTTGTCGCACCAGAGCGACATCGAAGTCACAGGTGACAG | |
| | | CTGCAATAATTACATGCTGACCTATAACAAGGTAGAGAACATGACAC | |
| | | CACGCGATTTGGGAGCCTGCATAGACTGTAGTGCTAGGCACATTAA | |
| | | TGCACAGGTCGCAAAATCTCATAACATCGCGCTAATCTGGAATGTC | |
| | | AAAGATTTCATGAGCCTGTCTGAGCAATTGAGAAAGCAAATCAGGA | |
| | | GTGCAGCTAAAAAAAATAACCTCCCGTTCAAGCTAACATGTGCCAC | |
| | | AACTAGACAGGTGGTGAACGTGGTAACTACTAAAATTGCGCTGAAG | |
| | | GGTGGTTGATAA | |
| 10 | Nsp4 | ATGAAAATTGTTAATAATTGGTTGAAGCAGTTAATTAAAGTTACACTT | 364 |
| | | GTGTTCCTTTTTGTTGCTGCTATTTTCTATTTAATAACACCTGTTCAT | |
| | | GTCATGTCTAAACATACTGACTTTTCAAGTGAAATCATAGGATACAA | |
| | | GGCTATTGATGGTGGTGTCACTCGTGACATAGCATCTACAGATACT | |
| | | TGTTTTGCTAACAAACATGCTGATTTTGACACATGGTTTAGCCAGCG | |
| | | TGGTGGTAGTTATACTAATGACAAAGCTTGCCCATTGATTGCTGCAG | |
| | | TCATAACAAGAGAAGTGGGTTTTGTCGTGCCTGGTTTGCCTGGCAC | |
| | | GATATTACGCACAACTAATGGTGACTTTTTGCATTTCTTACCTAGAG | |
| | | TTTTTAGTGCAGTTGGTAACATCTGTTACACACCATCAAAACTTATA | |
| | | GAGTACACTGACTTTGCAACATCAGCTTGTGTTTTGGCTGCTGAATG | |
| | | TACAATTTTTAAAGATGCTTCTGGTAAGCCAGTACCATATTGTTATGA | |
| | | TACCAATGTACTAGAAGGTTCTGTTGCTTATGAAAGTTTACGCCCTG | |
| | | ACACACGTTATGTGCTCATGGATGGCTCTATTATTCAATTTCCTAAC | |
| | | ACCTACCTTGAAGGTTCTGTTAGAGTGGTAACAACTTTTGATTCTGA | |
| | | GTACTGTAGGCACGGCACTTGTGAAAGATCAGAAGCTGGTGTTTGT | |
| | | GTATCTACTAGTGGTAGATGGGTACTTAACAATGATTATTACAGATC | |
| | | TTTACCAGGAGTTTTCTGTGGTGTAGATGCTGTAAATTTATTTACTAA | |
| | | TATGTTTACACCACTAATTCAACCTATTGGTGCTTTGGACATATCAG | |
| | | CATCTATAGTAGCTGGTGGTATTGTGGCTATCGTAGTAACATGCCTT | |
| | | GCCTACTATTTTATGAGGTTTAGAAGAGCTTTTGGTGAATACAGTCA | |
| | | TGTAGTTGCCTTTAATACTTTACTATTCCTTATGTCATTCATTGTACT | |
| | | CTGTTTAACACCAGTTTACTCATTCTTACCTGGTGTTTATTCTGTTAT | |
| | | TTACTTGTACTTGACATTTTATCTTACTAATGATGTTTCTTTTTTAGCA | |
| | | CATATTCAGTGGATGGTTATGTTCACACCTTTAGTACCTTTCTGGAT | |
| | | AACAATTGCTTATATCATTTGTATTTCCACAAAGCATTTCTATTGGTT | |
| | | CTTTAGTAATTACCTAAAGAGACGTGTAGTCTTTAATGGTGTTTCCTT | |
| | | TAGTACTTTTGAAGAAGCTGCGCTGTGCACCTTTTTGTTAAATAAAG | |
| | | AAATGTATCTAAAGTTGCGTAGTGATGTGCTATTACCTTTTACGCAA | |
| | | TATAATAGATACTTAGCTCTTTATAATAAGTACAAGTATTTTAGTGGA | |
| | | GCAATGGATACAACTAGCTACAGAGAAGCTGCTTGTTGTCATCTCG | |
| | | CAAAGGCTCTCAATGACTTCAGTAACTCAGGTTCTGATGTTCTTTAC | |
| | | CAACCACCACAAATCTCTATCACCTCAGCTGTTTTGCAGTGATGA | |
| 11 | Nsp4 | ATGAAAATCGTCAACAACTGGCTGAAACAGCTGATCAAAGTCACCTT | 365 |
| | (codon | AGTGTTCCTGTTCGTGGCAGCCATTTTCTATCTGATCACCCCTGTGC | |
| | optimized) | ATGTCATGTCAAAGCATACAGATTTTTCTAGTGAAATTATCGGTTATA | |
| | | AAGCTATCGACGGGGGGGTGACTAGGGACATAGCGTCGACTGATA | |
| | | CTTGCTTTGCAAACAAGCACGCAGATTTGATACCTGGTTTTCTCAA | |
| | | CGCGGAGGCTCCTACACAAATGACAAGGCCTGCCCACTCATCGCC | |
| | | GCTGTTATTACCAGAGAGGTAGGCTTCGTTGTTCCTGGGCTCCCAG | |
| | | GAACCATTCTGCGGACGACTAACGGAGATTTTCTCCACTTTCTGCC | |
| | | TAGAGTGTTTTCAGCTGTAGGAAACATATGTTACACACCCTCGAAGC | |
| | | TGATTGAGTACACCGACTTCGCAACTTCAGCATGTGTTCTCGCTGC | |
| | | AGAGTGCACCATCTTCAAGGACGCCTCCGGGAAGCCGGTCCCTTA | |
| | | CTGCTATGACACCAATGTGTTGGAAGGTTCCGTGGCCTATGAGTCA | |
| | | CTCCGTCCTGACACTCGGTATGTGTTGATGGATGGCAGCATCATTC | |
| | | AGTTTCCCAACACATATCTCGAGGGGAGTGTGAGGGTCGTGACCAC | |
| | | ATTTGACAGTGAATATTGCAGGCACGGAACCTGTGAACGATCCGAG | |
| | | GCCGGAGTATGCGTAAGCACATCAGGGAGGTGGGTACTCAATAAT | |
| | | GATTATTACCGTTCTTTGCCAGGGGTGTTCTGTGGTGTGGATGCGG | |
| | | TCAATCTCTTTACAAACATGTTTACTCCGTTAATTCAGCCAATAGGC | |
| | | GCTCTGGACATATCTGCTTCTATAGTAGCTGGCGGCATTGTTGCCA | |
| | | TTGTTGTGACATGCCTCGCATACTACTTCATGAGGTTCCGGCGAGC | |
| | | CTTCGGTGAGTATTCTCATGTGGTGGCGTTCAATACACTGCTGTTC | |
| | | CTGATGAGCTTCATTGTGCTGTGTCTGACACCAGTGTATTCCTTTTT | |
| | | GCCTGGTGTCTATTCTGTGATATACCTGTATTTGACCTTCTACCTTA | |
| | | CCAATGATGTGTCGTTCCTAGCGCACATCCAGTGGATGGTGATGTT | |
| | | CACTCCCTTAGTCCCCTTCTGGATCACAATCGCCTACATCATCTGCA | |
| | | TCAGCACCAAACATTTTTACTGGTTCTTCTCAAACTACCTGAAGCGC | |
| | | CGCGTGGTCTTTAATGGAGTCTCCTTCAGCACTTTTGAAGAGGCTG | |
| | | CCCTATGTACGTTTCTTCTCAACAAGGAGATGTACCTGAAACTAAGA | |
| | | TCTGATGTTCTGCTCCCATTCACGCAGTACAACAGATATCTTGCACT | |
| | | TTACAACAAATACAAGTACTTTAGTGGCGCCATGGACACAACGTCCT | |
| | | ACCGGGAAGCTGCTTGTTGTCACCTGGCCAAGGCCCTGAATGACTT | |
| | | TAGCAACAGCGGCAGTGACGTATTGTATCAGCCCCCCCAGATCTCC | |
| | | ATTACTAGCGCAGTGTTACAATGATAA | |
| 12 | Nsp5, | ATGAGTGGTTTTAGAAAAATGGCATTCCCATCTGGTAAAGTTGAGG | 366 |
| | Nsp6, | GTTGTATGGTACAAGTAACTTGTGGTACAACTACACTTAACGGTCTT | |
| | Nsp7, | TGGCTTGATGACGTAGTTTACTGTCCAAGACATGTGATCTGCACCT | |
| | Nsp8, | CTGAAGATATGCTTAACCCTAATTATGAAGATTTACTCATTCGTAAGT | |
| | Nsp9, | CTAATCATAATTTCTTGGTACAGGCTGGTAATGTTCAACTCAGGGTT | |
| | Nsp10 | ATTGGACATTCTATGCAAAATTGTGTACTTAAGCTTAAGGTTGATAC | |
| | | AGCCAATCCTAAGACACCTAAGTATAAGTTTGTTCGCATTCAACCAG | |
| | | GACAGACTTTTTCAGTGTTAGCTTGTTACAATGGTTCACCATCTGGT | |
| | | GTTTACCAATGTGCTATGAGACACAATTTCACTATTAAGGGTTCATT | |
| | | CCTTAATGGTTCATGTGGTAGTGTTGGTTTTAACATAGATTATGACT | |
| | | GTGTCTCTTTTTGTTACATGCACCATATGGAATTACCAACTGGAGTT | |
| | | CATGCTGGCACAGACTTAGAAGGTAACTTTTATGGACCTTTTGTTGA | |
| | | CAGGCAAACAGCACAAGCAGCTGGTACGGACACAACTATTACAGTT | |
| | | AATGTTTTAGCTTGGTTGTACGCTGCTGTTATAAATGGAGACAGGTG | |
| | | GTTTCTCAATCGATTTACCACAACTCTTAATGACTTTAACCTTGTGG | |
| | | CTATGAAGTACAATTATGAACCTCTAACACAAGACCATGTTGACATA | |
| | | CTAGGACCTCTTTCTGCTCAAACTGGAATTGCCGTTTTAGATATGTG | |
| | | TGCTTCATTAAAAGAATTACTGCAAAATGGTATGAATGGACGTACCA | |
| | | TATTGGGTAGTGCTTTATTAGAAGATGAATTTACACCTTTTGATGTTG | |
| | | TTAGACAATGCTCAGGTGTTACTTTCCAAAGTGCAGTGAAAAGAACA | |
| | | ATCAAGGGTACACACCACTGGTTGTTACTCACAATTTTGACTTCACT | |
| | | TTTAGTTTTAGTCCAGAGTACTCAATGGTCTTTGTTCTTTTTTTTGTA | |
| | | TGAAAATGCCTTTTTACCTTTTGCTATGGGTATTATTGCTATGTCTGC | |
| | | TTTTGCAATGATGTTTGTCAAACATAAGCATGCATTTCTCTGTTTGTT | |
| | | TTTGTTACCTTCTCTTGCCACTGTAGCTTATTTTAATATGGTCTATAT | |
| | | GCCTGCTAGTTGGGTGATGCGTATTATGACATGGTTGGATATGGTT | |
| | | GATACTAGTTTGAAGCTAAAAGACTGTGTTATGTATGCATCAGCTGT | |
| | | AGTGTTACTAATCCTTATGACAGCAAGAACTGTGTATGATGATGGTG | |
| | | CTAGGAGAGTGTGGACACTTATGAATGTCTTGACACTCGTTTATAAA | |
| | | GTTTATTATGGTAATGCTTTAGATCAAGCCATTTCCATGTGGGCTCT | |
| | | TATAATCTCTGTTACTTCTAACTACTCAGGTGTAGTTACAACTGTCAT | |
| | | GTTTTTGGCCAGAGGTATTGTTTTTATGTGTGTTGAGTATTGCCCTA | |
| | | TTTTCTTCATAACTGGTAATACACTTCAGTGTATAATGCTAGTTTATT | |
| | | GTTTCTTAGGCTATTTTTGTACTTGTTACTTTGGCCTCTTTTGTTTAC | |
| | | TCAACCGCTACTTTAGACTGACTCTTGGTGTTTATGATTACTTAGTTT | |
| | | CTACACAGGAGTTTAGATATATGAATTCACAGGGACTACTCCCACC | |
| | | CAAGAATAGCATAGATGCCTTCAAACTCAACATTAAATTGTTGGGTG | |
| | | TTGGTGGCAAACCTTGTATCAAAGTAGCCACTGTACAGTCTAAAATG | |
| | | TCAGATGTAAAGTGCACATCAGTAGTCTTACTCTCAGTTTTGCAACA | |
| | | ACTCAGAGTAGAATCATCATCTAAATTGTGGGCTCAATGTGTCCAGT | |
| | | TACACAATGACATTCTCTTAGCTAAAGATACTACTGAAGCCTTTGAA | |
| | | AAAATGGTTTCACTACTTTCTGTTTTGCTTTCCATGCAGGGTGCTGT | |
| | | AGACATAAACAAGCTTTGTGAAGAAATGCTGGACAACAGGGCAACC | |
| | | TTACAAGCTATAGCCTCAGAGTTTAGTTCCCTTCCATCATATGCAGC | |
| | | TTTTGCTACTGCTCAAGAAGCTTATGAGCAGGCTGTTGCTAATGGT | |
| | | GATTCTGAAGTTGTTCTTAAAAAGTTGAAGAAGTCTTTGAATGTGGC | |
| | | TAAATCTGAATTTGACCGTGATGCAGCCATGCAACGTAAGTTGGAA | |
| | | AAGATGGCTGATCAAGCTATGACCCAAATGTATAAACAGGCTAGAT | |
| | | CTGAGGACAAGAGGGCAAAAGTTACTAGTGCTATGCAGACAATGCT | |
| | | TTTCACTATGCTTAGAAAGTTGGATAATGATGCACTCAACAACATTA | |
| | | TCAACAATGCAAGAGATGGTTGTGTTCCCTTGAACATAATACCTCTT | |
| | | ACAACAGCAGCCAAACTAATGGTTGTCATACCAGACTATAACACATA | |
| | | TAAAAATACGTGTGATGGTACAACATTTACTTATGCATCAGCATTGT | |
| | | GGGAAATCCAACAGGTTGTAGATGCAGATAGTAAAATTGTTCAACTT | |
| | | AGTGAAATTAGTATGGACAATTCACCTAATTTAGCATGGCCTCTTAT | |
| | | TGTAACAGCTTTAAGGGCCAATTCTGCTGTCAAATTACAGAATAATG | |
| | | AGCTTAGTCCTGTTGCACTACGACAGATGTCTTGTGCTGCCGGTAC | |
| | | TACACAAACTGCTTGCACTGATGACAATGCGTTAGCTTACTACAACA | |
| | | CAACAAAGGGAGGTAGGTTTGTACTTGCACTGTTATCCGATTTACA | |
| | | GGATTTGAAATGGGCTAGATTCCCTAAGAGTGATGGAACTGGTACT | |
| | | ATTTATACAGAACTGGAACCACCTTGTAGGTTTGTTACAGACACACC | |
| | | TAAAGGTCCTAAAGTGAAGTATTTATACTTTATTAAAGGATTAAACAA | |
| | | CCTAAATAGAGGTATGGTACTTGGTAGTTTAGCTGCCACAGTACGT | |
| | | CTACAAGCTGGTAATGCAACAGAAGTGCCTGCCAATTCAACTGTATT | |
| | | ATCTTTCTGTGCTTTTGCTGTAGATGCTGCTAAAGCTTACAAAGATT | |
| | | ATCTAGCTAGTGGGGGACAACCAATCACTAATTGTGTTAAGATGTTG | |
| | | TGTACACACACTGGTACTGGTCAGGCAATAACAGTTACACCGGAAG | |
| | | CCAATATGGATCAAGAATCCTTTGGTGGTGCATCGTGTTGTCTGTAC | |
| | | TGCCGTTGCCACATAGATCATCCAAATCCTAAAGGATTTTGTGACTT | |
| | | AAAAGGTAAGTATGTACAAATACCTACAACTTGTGCTAATGACCCTG | |
| | | TGGGTTTTACACTTAAAAACACAGTCTGTACCGTCTGCGGTATGTG | |
| | | GAAAGGTTATGGCTGTAGTTGTGATCAACTCCGCGAACCCATGCTT | |
| | | CAGTGATGA | |
| 13 | Nsp5_{,} | ATGAGTGGCTTCCGGAAGATGGCTTTTCCATCAGGCAAAGTGGAAG | 367 |
| | Nsp6, | GCTGCATGGTCCAGGTGACCTGTGGAACCACTACATTGAACGGCCT | |
| | Nsp7, | CTGGCTGGATGACGTCGTATACTGTCCTAGACACGTGATCTGCACG | |
| | Nsp8, | TCAGAGGACATGCTCAATCCTAATTATGAAGATCTACTCATACGGAA | |
| | Nsp9, | GTCGAACCATAATTTCTTGGTGCAAGCAGGGAATGTGCAGCTGCGC | |
| | Nsp10 | GTCATCGGCCACAGCATGCAAAACTGTGTACTGAAGTTGAAGGTGG | |
| | (codon | ACACTGCCAATCCTAAAACCCCCAAGTATAAGTTCGTGAGGATCCA | |
| | optimized) | GCCAGGACAAACTTTCAGTGTACTGGCGTGCTACAATGGCTCCCCT | |
| | | TCTGGCGTCTATCAGTGTGCTATGCGCCATAATTTTACGATTAAGGG | |
| | | TTCCTTCCTAAACGGATCATGCGGCAGTGTGGGCTTTAATATAGACT | |
| | | ACGACTGCGTGAGTTTCTGCTATATGCATCACATGGAGCTACCAAC | |
| | | AGGGGTCCATGCTGGAACTGACCTGGAGGGCAATTTCTATGGGCC | |
| | | TTTTGTGGATCGTCAGACAGCACAAGCCGCCGGCACCGACACCAC | |
| | | CATTACGGTAAATGTGTTAGCCTGGCTTTATGCTGCTGTAATCAATG | |
| | | GAGACCGTTGGTTCCTGAACCGCTTTACGACAACCCTCAATGACTT | |
| | | CAATCTAGTAGCCATGAAGTACAACTATGAGCCCTTAACACAGGAC | |
| | | CACGTGGATATTCTGGGCCCGCTTAGCGCCCAGACCGGCATCGCG | |
| | | GTTCTGGATATGTGTGCATCACTGAAAGAGCTTCTGCAGAACGGAA | |
| | | TGAATGGAAGGACCATATTGGGTTCTGCGTTGCTTGAAGATGAGTT | |
| | | CACCCCGTTTGATGTTGTGCGCCAGTGTAGCGGGGTCACCTTTCAG | |
| | | TCTGCTGTGAAGCGGACTATAAAGGGAACACATCACTGGTTACTGC | |
| | | TGACGATACTGACGTCTCTGCTGGTACTGGTACAGAGTACCCAGTG | |
| | | GTCTTTGTTCTTTTTTCTCTACGAAAACGCGTTCCTGCCCTTTGCGA | |
| | | TGGGCATTATTGCTATGTCCGCGTTCGCCATGATGTTTGTGAAGCA | |
| | | CAAACATGCCTTCCTCTGCTTGTTTTTGCTCCCTTCTCTAGCAACTG | |
| | | TGGCCTATTTCAACATGGTTTACATGCCAGCAAGCTGGGTGATGAG | |
| | | GATTATGACCTGGCTTGACATGGTTGACACATCTCTGAAGCTTAAG | |
| | | GACTGTGTCATGTACGCCAGCGCTGTAGTTCTGCTGATTCTCATGA | |
| | | CAGCTCGCACCGTCTACGATGATGGGGCCAGACGAGTGTGGACAT | |
| | | TAATGAACGTGTTAACTTTGGTTTACAAGGTTTACTATGGAAATGCC | |
| | | CTTGATCAGGCAATCAGCATGTGGGCATTAATCATCTCAGTCACTA | |
| | | GCAACTACAGTGGGGTCGTTACCACAGTGATGTTCCTAGCTCGAGG | |
| | | AATCGTGTTCATGTGCGTGGAGTACTGTCCCATCTTCTTCATCACTG | |
| | | GTAACACATTGCAGTGCATCATGCTGGTATATTGCTTCCTGGGCTA | |
| | | CTTCTGTACATGTTACTTTGGACTATTTTGCCTGCTGAACCGCTACT | |
| | | TCCGGCTGACGTTGGGAGTGTACGACTACCTCGTCTCGACCCAGG | |
| | | AGTTCAGGTACATGAATAGTCAGGGCCTCCTGCCGCCCAAGAACTC | |
| | | CATTGACGCTTTCAAGCTGAACATCAAACTTCTTGGGGTGGGTGGC | |
| | | AAGCCCTGTATTAAGGTGGCCACTGTGCAATCCAAGATGTCTGACG | |
| | | TCAAGTGTACCAGCGTCGTGTTACTGTCAGTGCTACAGCAGTTACG | |
| | | AGTGGAAAGTTCTAGCAAGCTTTGGGCTCAATGCGTCCAGCTGCAC | |
| | | AATGACATTCTTCTAGCCAAAGACACCACTGAAGCTTTTGAGAAAAT | |
| | | GGTCTCGTTGCTCTCCGTGCTGTTGTCCATGCAGGGGGCTGTGGA | |
| | | CATCAACAAGCTATGTGAAGAGATGCTGGACAACAGAGCTACCCTC | |
| | | CAAGCCATTGCCTCAGAATTTTCCTCCCTACCCTCTTACGCTGCATT | |
| | | TGCTACAGCGCAGGAAGCCTACGAGCAGGCAGTGGCAAACGGTGA | |
| | | TAGTGAAGTAGTTCTGAAAAAGCTGAAGAAGTCCCTGAATGTCGCA | |
| | | AAATCCGAGTTTGACAGAGATGCCGCTATGCAGCGGAAACTGGAGA | |
| | | AGATGGCTGACCAGGCAATGACTCAGATGTATAAGCAGGCAAGGTC | |
| | | AGAGGACAAAAGGGCTAAGGTCACATCGGCTATGCAGACGATGCT | |
| | | GTTTACCATGCTCAGAAAACTCGACAATGACGCGCTCAACAATATTA | |
| | | TCAATAACGCCCGCGATGGCTGTGTTCCACTAAACATCATCCCCCT | |
| | | TACCACTGCTGCTAAACTCATGGTTGTGATTCCAGATTACAACACCT | |
| | | ATAAAAATACTTGCGATGGTACCACATTTACATATGCCAGTGCCCTC | |
| | | TGGGAGATTCAGCAAGTGGTGGATGCCGACAGCAAAATTGTGCAG | |
| | | CTTTCTGAAATCTCTATGGATAACAGCCCCAACCTAGCTTGGCCACT | |
| | | GATCGTGACTGCACTCCGGGCCAATTCCGCAGTCAAGTTGCAGAAT | |
| | | AATGAGCTGTCCCCTGTTGCCCTTCGACAGATGAGCTGCGCAGCA | |
| | | GGAACAACACAGACAGCCTGTACAGATGATAATGCTTTGGCCTATT | |
| | | ACAACACAACCAAGGGTGGAAGATTTGTCCTGGCCCTGCTTTCAGA | |
| | | CCTGCAAGACCTTAAGTGGGCCAGATTCCCTAAGTCTGACGGGACT | |
| | | GGTACTATCTATACCGAATTGGAGCCTCCATGCAGGTTCGTCACTG | |
| | | ATACTCCCAAAGGGCCGAAGGTGAAATATTTATATTTTATCAAGGGA | |
| | | TTAAATAACCTGAACCGGGGCATGGTGCTGGGGAGCTTGGCAGCC | |
| | | ACGGTAAGGCTACAAGCTGGAAACGCCACAGAGGTTCCAGCGAAC | |
| | | AGTACAGTTCTGAGCTTCTGTGCGTTCGCCGTAGATGCAGCAAAAG | |
| | | CATACAAAGATTACCTGGCCAGCGGAGGACAGCCTATCACAAACTG | |
| | | CGTCAAAATGCTGTGCACCCACACGGGAACTGGACAGGCCATAACT | |
| | | GTTACCCCAGAGGCTAACATGGACCAGGAGTCTTTTGGGGGTGCTT | |
| | | CCTGCTGTCTGTACTGCCGTTGCCACATTGATCATCCAAACCCTAAA | |
| | | GGGTTTTGTGATCTTAAGGGTAAATATGTGCAGATACCTACAACATG | |
| | | CGCCAACGATCCCGTGGGGTTCACTCTGAAAAATACCGTGTGTACC | |
| | | GTCTGTGGCATGTGGAAAGGCTATGGTTGCAGCTGCGATCAACTCA | |
| | | GAGAACCCATGCTTCAATGATAA | |
| 14 | Nsp12 | ATGTCAGCTTGCACAATCGTTTTTAAACGGGTTTGCGGTGTAAGTG | 368 |
| | | CAGCCCGTCTTACACCGTGCGGCACAGGCACTAGTACTGATGTCGT | |
| | | ATACAGGGCTTTTGACATCTACAATGATAAAGTAGUGGTTTTGCTA | |
| | | AATTCCTAAAAACTAATTGTTGTCGCTTCCAAGAAAAGGACGAAGAT | |
| | | GACAATTTAATTGATTCTTACTTTGTAGTTAAGAGACACACTTTCTCT | |
| | | AACTACCAACATGAAGAAACAATTTATAATTTACTTAAGGATTGTCCA | |
| | | GCTGTTGCTAAACATGACTTCTTTAAGTTTAGAATAGACGGTGACAT | |
| | | GGTACCACATATATCACGTCAACGTCTTACTAAATACACAATGGCAG | |
| | | ACCTCGTCTATGCTTTAAGGCATTTTGATGAAGGTAATTGTGACACA | |
| | | TTAAAAGAAATACTTGTCACATACAATTGTTGTGATGATGATTATTTC | |
| | | AATAAAAAGGACTGGTATGATTTTGTAGAAAACCCAGATATATTACG | |
| | | CGTATACGCCAACTTAGGTGAACGTGTACGCCAAGCTTTGTTAAAA | |
| | | ACAGTACAATTCTGTGATGCCATGCGAAATGCTGGTATTGTTGGTGT | |
| | | ACTGACATTAGATAATCAAGATCTCAATGGTAACTGGTATGATTTCG | |
| | | GTGATTTCATACAAACCACGCCAGGTAGTGGAGTTCCTGTTGTAGA | |
| | | TTCTTATTATTCATTGTTAATGCCTATATTAACCTTGACCAGGGCTTT | |
| | | AACTGCAGAGTCACATGTTGACACTGACTTAACAAAGCCTTACATTA | |
| | | AGTGGGATTTGTTAAAATATGACTTCACGGAAGAGAGGTTAAAACTC | |
| | | TTTGACCGTTATTTTAAATATTGGGATCAGACATACCACCCAAATTG | |
| | | TGTTAACTGTTTGGATGACAGATGCATTCTGCATTGTGCAAACTTTA | |
| | | ATGTTTTATTCTCTACAGTGTTCCCACTTACAAGMTTGGACCACTA | |
| | | GTGAGAAAAATATTTGTTGATGGTGTTCCATTTGTAGTTTCAACTGG | |
| | | ATACCACTTCAGAGAGCTAGGTGTTGTACATAATCAGGATGTAAACT | |
| | | TACATAGCTCTAGACTTAGTTTTAAGGAATTACTTGTGTATGCTGCT | |
| | | GACCCTGCTATGCACGCTGCTTCTGGTAATCTATTACTAGATAAACG | |
| | | CACTACGTGCTTCAGTAGCTGCACTTACTAACAATGTTGCTTTTC | |
| | | AAACTGTCAAACCCGGTAATTAACAAAGACTTCTATGACTTTGCT | |
| | | GTGTCTAAGGGTTTCTTTAAGGAAGGAAGTTCTGTTGAATTAAAACA | |
| | | CTTCTTCTTTGCTCAGGATGGTAATGCTGCTATCAGCGATTATGACT | |
| | | ACTATCGTTATAATCTACCAACAATGTGTGATATCAGACAACTACTAT | |
| | | TTGTAGTTGAAGTTGTTGATAAGTACTTTGATTGTTACGATGGTGGC | |
| | | TGTATTAATGCTAACCAAGTCATCGTCAACAACCTAGACAAATCAGC | |
| | | TGGTTTTCCATTTAATAAATGGGGTAAGGCTAGACTTTATTATGATTC | |
| | | AATGAGTTATGAGGATCAAGATGCACTTTTCGCATATACAAAACGTA | |
| | | ATGTCATCCCTACTATAACTCAAATGAATCTTAAGTATGCCATTAGT | |
| | | GCAAAGAATAGAGCTCGCACCGTAGCTGGTGTCTCTATCTGTAGTA | |
| | | CTATGACCAATAGACAGTTTCATCAAAAATTATTGAAATCAATAGCC | |
| | | GCCACTAGAGGAGCTACTGTAGTAATTGGAACAAGCAAATTCTATG | |
| | | GTGGTTGGCACAACATGTTAAAAACTGTTTATAGTGATGTAGAAAAC | |
| | | CCTCACCTTATGGGTTGGGATTATCCTAAATGTGATAGAGCCATGC | |
| | | CTAACATGCTTAGAATTATGGCCTCACTTGTTCTTGCTCGCAAACAT | |
| | | ACAACGTGTTGTAGCTTGTCACACCGTTTCTATAGATTAGCTAATGA | |
| | | GTGTGCTCAAGTATTGAGTGAAATGGTCATGTGTGGCGGTTCACTA | |
| | | TATGTTAAACCAGGTGGAACCTCATCAGGAGATGCCACAACTGCTT | |
| | | ATGCTAATAGTGTTTTTAACATTTGTCAAGCTGTCACGGCCAATGTT | |
| | | AATGCACTTTTATCTACTGATGGTAACAAAATTGCCGATAAGTATGT | |
| | | CCGCAATTTACAACACAGACTTTATGAGTGTCTCTATAGAAATAGAG | |
| | | ATGTTGACACAGACTTTGTGAATGAGTTTTACGCATATTTGCGTAAA | |
| | | CATTTCTCAATGATGATACTTTCTGACGATGCTGTTGTGTGTTTCAAT | |
| | | AGCACTTATGCATCTCAAGGTCTAGTGGCTAGCATAAAGAACTTTAA | |
| | | GTCAGTTCTTTATTATCAAAACAATGTTTTTATGTCTGAAGCAAAATG | |
| | | TTGGACTGAGACTGACCTTACTAAAGGACCTCATGAATTGCTCTC | |
| | | AACATACAATGCTAGTTAAACAGGGTGATGATTATGTGTACCTTCCT | |
| | | TACCCAGATCCATCAAGAATCCTAGGGGCCGGCTGTTTTGTAGATG | |
| | | ATATCGTAAAAACAGATGGTACACTTATGATTGAACGGTTCGTGTCT | |
| | | TTAGCTATAGATGCTTACCCACTTACTAAACATCCTAATCAGGAGTA | |
| | | TGCTGATGTCTTTCATTTGTACTTACAATACATAAGAAAGCTACATGA | |
| | | TGAGTTAACAGGACACATGTTAGACATGTATTCTGTTATGCTTACTA | |
| | | ATGATAACACTTCAAGGTATTGGGAACCTGAGTTTTATGAGGCTATG | |
| | | TACACACCGCATACAGTCTTACAGTGATGA | |
| 15 | Nsp | ATGTCTGCCTGCACAATTGTGTTCAAGCGGGTGTGTGGAGTGTCTG | 369 |
| | (codon | CAGCGCGATTAACTCCCTGTGGAACCGGCACCTCAACAGACGTAGT | |
| | optimized) | GTACCGTGCCTTCGATATTTACAATGATAAGGTGGCCGGGTTCGCG | |
| | | AAATTCCTAAAGACGAACTGTTGCAGGTTCCAGGAGAAGGATGAAG | |
| | | ATGACAATCTCATAGATTCTTATTTCGTGGTTAAACGGCATACATTTA | |
| | | GTAATTACCAACATGAAGAAACAATCTACAACCTCCTCAAAGACTGT | |
| | | CCTGCTGTGGCAAAACATGACTTCTTCAAGTTCCGGATTGACGGCG | |
| | | ACATGGTTCCACACATCTCTCGGCAGAGATTAACAAAGTACACCAT | |
| | | GGCTGACCTTGTATATGCACTGCGACACTTTGATGAAGGAAATTGC | |
| | | GATACTCTGAAAGAGATTCTTGTTACCTACAACTGCTGTGATGACGA | |
| | | CTACTTCAACAAGAAAGACTGGTACGACTTTGTAGAAAACCCAGATA | |
| | | TCCTCAGAGTTTACGCCAACTTAGGAGAGCGCGTAAGACAAGCCCT | |
| | | GTTAAAAACAGTTCAGTTCTGTGATGCCATGAGGAATGCAGGAATC | |
| | | GTGGGAGTCTTGACCCTGGACAACCAGGACTTGAATGGGAACTGG | |
| | | TATGACTTCGGGGATTTCATCCAGACTACACCCGGCAGCGGAGTGC | |
| | | CAGTGGTGGATAGCTATTACTCCTTGCTGATGCCCATCCTTACGTT | |
| | | GACAAGAGCCCTGACAGCAGAGAGCCATGTGGACACTGACCTTAC | |
| | | CAAACCCTACATCAAGTGGGACTTACTCAAGTATGACTTCACAGAA | |
| | | GAGCGGCTAAAGTTGTTCGATCGCTACTTTAAATATTGGGATCAGA | |
| | | CCTATCATCCCAACTGTGTGAATTGTCTGGATGACAGGTGCATATTG | |
| | | CACTGTGCAAACTTCAACGTTCTTTTTTCCACGGTTTTCCCCCTGAC | |
| | | CAGCTTTGGGCCTCTGGTGAGAAAGATCTTTGTCGACGGGGTACCA | |
| | | TTTGTTGTCAGTACCGGCTACCATTTCAGGGAACTCGGTGTCGTGC | |
| | | ACAATCAGGATGTAAACCTGCACAGCAGCCGGCTAAGCTTTAAAGA | |
| | | ACTGCTTGTTTATGCTGCTGATCCAGCCATGCACGCTGCCAGCGGA | |
| | | AATTTACTCCTGGACAAGCGTACTACCTGTTTCAGTGTGGCAGCCC | |
| | | TCACCAACAACGTAGCCTTCCAAACCGTGAAACCAGGCAACTTTAA | |
| | | CAAAGATTTCTACGACTTTGCCGTTTCAAAGGGGTTTTTTAAGGAGG | |
| | | GGAGTTCCGTGGAACTGAAGCACTTTGCACAAGACGGTAAT | |
| | | GCTGCTATAAGCGACTATGATTATTACCGGTATAATCTGCCAACGAT | |
| | | GTGTGATATTAGGCAACTGCTCTTCGTGGTAGAGGTCGTGGACAAA | |
| | | TACTTTGACTGCTACGATGGCGGCTGCATCAATGCTAATCAGGTGA | |
| | | TTGTCAACAATCTCGACAAGAGTGCGGGCTTTCCTTTCAATAAATGG | |
| | | GGCAAGGCCCGCCTCTACTATGACAGCATGAGCTACGAGGACCAA | |
| | | GATGCTCTGTTTGCTTATACAAAGAGAAATGTGATCCCTACCATCAC | |
| | | ACAGATGAACCTTAAATATGCCATCTCCGCGAAGAACCGTGCCCGA | |
| | | ACAGTTGCTGGTGTCTCCATTTGTTCTACCATGACAAACCGCCAGTT | |
| | | CCACCAGAAGCTGTTGAAGAGCATAGCAGCTACTAGGGGCGCCAC | |
| | | CGTCGTAATCGGGACATCCAAGTTTTATGGAGGGTGGCACAACATG | |
| | | CTTAAAACGGTTTACTCAGATGTGGAGAATCCCCACCTAATGGGCT | |
| | | GGGACTACCCCAAGTGTGACCGAGCAATGCCGAACATGCTCCGCA | |
| | | TTATGGCAAGTCTGGTCCTCGCCAGAAAACACACGACTTGCTGCTC | |
| | | ATTGTCACACAGGTTTTATCGTCTGGCCAATGAATGTGCCCAGGTC | |
| | | CTGTCAGAGATGGTCATGTGCGGAGGATCCTTATATGTGAAGCCAG | |
| | | GTGGCACTAGTTCTGGTGATGCCACAACTGCGTACGCAAATAGTGT | |
| | | GTTCAACATCTGCCAGGCTGTCACTGCGAATGTTAATGCGTTGCTG | |
| | | TCCACTGATGGAAACAAAATTGCAGACAAATACGTGCGCAACCTGC | |
| | | AGCATCGTTTATATGAGTGCCTATACAGAAACAGAGATGTGGACAC | |
| | | CGATTTTGTGAACGAGTTCTATGCCTACCTGAGGAAGCATTTCTCAA | |
| | | TGATGATCCTGTCGGATGATGCCGTTGTCTGCTTTAATTCTACTTAT | |
| | | GCTTCACAGGGCCTGGTGGCTTCCATAAAAAACTTCAAGTCTGTGC | |
| | | TGTATTACCAGAACAATGTGTTTATGTCTGAAGCTAAGTGCTGGACC | |
| | | GAGACGGACCTCACCAAGGGTCCCCATGAGTTCTGCTCCCAGCAC | |
| | | ACAATGCTTGTGAAGCAGGGGGACGATTATGTGTATCTCCCTTACC | |
| | | CTGACCCTTCGAGGATACTGGGCGCCGGGTGTTTTGTAGACGACAT | |
| | | TGTCAAAACTGACGGTACTCTAATGATCGAGCGGTTCGTGAGCCTG | |
| | | GCTATTGATGCTTACCCACTGACCAAGCACCCGAATCAAGAGTACG | |
| | | CAGATGTCTTTCACCTTTACCTTCAGTACATTCGGAAGCTTCATGAT | |
| | | GAGCTGACCGGCCACATGCTGGACATGTATTCAGTGATGCTGACAA | |
| | | ATGACAATACATCTAGGTACTGGGAGCCTGAATTTTATGAAGCTATG | |
| | | TATACTCCTCATACCGTGTTGCAGTGATAA | |
| 16 | Nsp13 | ATGGCTGAAAATGTAACAGGACTCTTTAAAGATTGTAGTAAGGTAAT | 370 |
| | | CACTGGGTTACATCCTACACAGGCACCTACACACCTCAGTGTTGAC | |
| | | ACTAAATTCAAAACTGAAGGTTTATGTGTTGACGTACCTGGCATACC | |
| | | TAAGGACATGACCTATAGAAGACTCATCTCTATGATGGGTTTTAAAA | |
| | | TGAATTATCAAGTTAATGGTTACCCTAACATGTTTATCACCCGCGAA | |
| | | GAAGCTATAAGACATGTACGTGCATGGATTGGCTTCGATGTCGAGG | |
| | | GGTGTCATGCTACTAGAGAAGCTGTTGGTACCAATTTACCTTTACAG | |
| | | CTAGGTTTTTCTACAGGTGTTAACCTAGTTGCTGTACCTACAGGTTA | |
| | | TGTTGATACACCTAATAATACAGATTTTTCCAGAGTTAGTGCTAAAC | |
| | | CACCGCCTGGAGATCAATTTAAACACCTCATACCACTTATGTACAAA | |
| | | GGACTTCCTTGGAATGTAGTGCGTATAAAGATTGTACAAATGTTAAG | |
| | | TGACACACTTAAAAATCTCTCTGACAGAGTCGTATTTGTCTTATGGG | |
| | | CACATGGCTTTGAGTTGACATCTATGAAGTATTTTGTGAAAATAGGA | |
| | | CCTGAGCGCACCTGTTGTCTATGTGATAGACGTGCCACATGCTTTT | |
| | | CCACTGCTTCAGACACTTATGCCTGTTGGCATCATTCTATTGGATTT | |
| | | GATTACGTCTATAATCCGTTTATGATTGATGTTCAACAATGGGGTTT | |
| | | TACAGGTAACCTACAAAGCAACCATGATCTGTATTGTCAAGTCCATG | |
| | | GTAATGCACATGTAGCTAGTTGTGATGCAATCATGACTAGGTGTCTA | |
| | | GCTGTCCACGAGTGCTTTGTTAAGCGTGTTGACTGGACTATTGAAT | |
| | | ATCCTATAATTGGTGATGAACTGAAGATTAATGCGGCTTGTAGAAAG | |
| | | GTTCAACACATGGTTGTTAAAGCTGCATTATTAGCAGACAAATTCCC | |
| | | AGTTCTTCACGACATTGGTAACCCTAAAGCTATTAAGTGTGTACCTC | |
| | | AAGCTGATGTAGAATGGAAGTTCTATGATGCACAGCCTTGTAGTGA | |
| | | CAAAGCTTATAAAATAGAAGAATTATTCTATTCTTATGCCACACATTC | |
| | | TGACAAATTCACAGATGGTGTATGCCTATTTGGAATTGCAATGTCG | |
| | | ATAGATATCCTGCTAATTCCATTGTTTGTAGATTTGACACTAGAGTG | |
| | | CTATCTAACCTTAACTTGCCTGGTTGTGATGGTGGCAGTTTGTATGT | |
| | | AAATAAACATGCATTCCACACACCAGCTTTTGATAAAAGTGCTTG | |
| | | TTAATTTAAAACAATTACCATTTTTCTATTACTCTGACAGTCCATGTG | |
| | | AGTCTCATGGAAAACAAGTAGTGTCAGATATAGATTATGTACCACTA | |
| | | AAGTCTGCTACGTGTATAACACGTTGCAATTTAGGTGGTGCTGTCT | |
| | | GTAGACATCATGCTAATGAGTACAGATTGTATCTCGATGCTTATAAC | |
| | | ATGATGATCTCAGCTGGCTTTAGCTTGTGGGTTTACAAACAATTTGA | |
| | | TACTTATAACCTCTGGAACACTTTTACAAGACTTCAGTGATGA | |
| 17 | Nsp | ATGGCTGAGAATGTCACAGGGCTGTTTAAGGACTGTTCCAAGGTGA | 371 |
| | (codon | TAACAGGACTGCACCCAACACAGGCCCCTACGCACTTGTCTGTTGA | |
| | optimized) | CACAAAATTCAAAACAGAAGGACTCTGTGTGGATGTACCCGGCATC | |
| | | CCTAAAGACATGACCTATCGCAGGCTCATCAGTATGATGGGCTTCA | |
| | | AGATGAACTACCAGGTCAATGGCTACCCAAATATGTTCATCACCAG | |
| | | GGAGGAAGCTATTAGGCATGTGAGGGCCTGGATTGGTTTTGACGT | |
| | | GGAAGGCTGTCATGCAACAAGAGAAGCAGTGGGGACCAACTTACC | |
| | | TCTGCAGCTGGGGTTCAGCACTGGGGTCAACCTGGTTGCTGTCCC | |
| | | AACTGGATATGTTGATACCCCCAACAACACAGACTTCTCACGGGTC | |
| | | TCTGCAAAGCCTCCACCTGGGGATCAGTTCAAGCACCTGATCCCAT | |
| | | TAATGTATAAAGGTCTGCCCTGGAATGTGGTAAGGATCAAAATTGTT | |
| | | CAGATGCTGAGTGACACTCTGAAGAACTTGAGTGACAGAGTTGTGT | |
| | | TTGTCCTGTGGGCTCATGGCTTTGAGCTGACCAGCATGAAGTACTT | |
| | | TGTGAAAATTGGACCAGAGAGAACGTGCTGCCTGTGTGACCGACG | |
| | | GGCAACTTGTTTTTCTACCGCATCAGATACTTACGCCTGCTGGCAC | |
| | | CATAGTATCGGCTTTGACTATGTCTACAACCCTTTCATGATTGATGT | |
| | | CCAGCAATGGGGCTTCACAGGAAATTTGCAGTCCAACCACGACCTG | |
| | | TACTGCCAGGTCCATGGGAATGCTCACGTGGCCTCCTGCGATGCTA | |
| | | TCATGACACGATGTCTGGCTGTGCATGAGTGCTTTGTGAAGCGTGT | |
| | | CGACTGGACCATCGAATACCCCATTATTGGCGACGAGCTAAAAATA | |
| | | AATGCAGCGTGTAGAAAAGTGCAGCACATGGTGGTCAAGGCAGCA | |
| | | CTCCTTGCTGACAAGTTTCCAGTATTACATGACATCGGCAACCCAAA | |
| | | GGCCATTAAGTGTGTTCCTCAAGCGGATGTGGAGTGGAAGTTCTAT | |
| | | GATGCCCAGCCGTGCTCTGATAAAGCCTACAAGATAGAAGAACTCT | |
| | | TTTATTCTTACGCTACTCACAGCGACAAATTTACAGATGGAGTTTGC | |
| | | CTCTTCTGGAACTGCAATGTCGATAGATATCCGGCCAACAGCATAG | |
| | | TGTGCCGCTTTGATACGCGCGTGCTATCCAACTTGAACCTCCCGGG | |
| | | TTGTGATGGCGGTTCGCTTTATGTAAATAAACATGCTTTCCACACAC | |
| | | CTGCCTTCGACAAGTCCGCCTTTGTGAATCTGAAACAACTTCCCTTC | |
| | | TTCTACTACAGCGACAGCCCCTGCGAGTCCCACGGGAAGCAGGTG | |
| | | GTGAGTGATATTGACTATGTCCCCCTTAAGTCAGCGACTTGCATCA | |
| | | CTCGCTGTAACCTTGGAGGAGCTGTTTGTCGGCACCACGCGAATGA | |
| | | GTACCGACTCTACCTGGACGCCTATAACATGATGATCTCTGCCGGT | |
| | | TTCTCACTATGGGTATATAAGCAGTTTGATACCTACAATTTGTGGAA | |
| | | CACCTTCACCCGGCTCCAATGATAA | |
| 18 | Or7a/7b | ATGAAAATTATTCTTTTCTTGGCACTGATAACACTCGCTACTTGTGA | 372 |
| | | GCTTTATCACTACCAAGAGTGTGTTAGAGGTACAACAGTACTTTTAA | |
| | | AAGAACCTTGCTCTTCTGGAACATACGAGGGCAATTCACCATTTCAT | |
| | | CCTCTAGCTGATAACAAATTTGCACTGACTTGCTTTAGCACTCAATT | |
| | | TGCTTTTGCTTGTCCTGACGGCGTAAAACACGTCTATCAGTTACGTG | |
| | | CCAGATCAGTTTCACCTAAACTGTTCATCAGACAAGAGGAAGTTCAA | |
| | | GAACTTTACTCTCCAATTTTTCTTATTGTTGCGGCAATAGTGTTTATA | |
| | | ACACTTTGCTTCACACTCAAAAGAAAGACAGAATGTGAACTTTCATT | |
| | | AATTGACTTCTATTTGTGCTTTTTAGCCTTTCTGTTATTCCTTGTTTA | |
| | | ATTATGCTTATTATCTTTTGGTTCTCACTTGAACTGCAAGATCATAAT | |
| | | GAAACTTGTCACGCCTAATGATGA | |
| 19 | Orf7a/7b | ATGAAGATCATTTTGTTTCTAGCATTAATAACTCTAGCCACCTGTGA | 373 |
| | (codon | GCTCTACCACTACCAGGAGTGTGTGAGGGGTACCACTGTACTGCTG | |
| | optimized) | AAGGAGCCCTGCAGCTCTGGAACATATGAAGGCAACAGCCCTTTCC | |
| | | ACCCTTTGGCTGATAACAAGTTTGCTCTTACGTGCTTTTCTACTCAG | |
| | | TTTGCATTTGCCTGCCCAGATGGGGTGAAGCATGTGTATCAGCTGC | |
| | | GAGCGCGCAGTGTTTCCCCAAAACTCTTCATTCGGCAAGAAGAGGT | |
| | | CCAAGAACTGTATAGTCCCATCTTTCTCATTGTGGCTGCCATCGTGT | |
| | | TCATCACACTATGTTTCACCCTGAAAAGAAAAACAGAATGTGAACTT | |
| | | TCATTGATTGACTTCTACCTGTGCTTCCTGGCCTTCCTCTTATTTCTT | |
| | | GTTCTCATCATGCTGATCATCTTCTGGTTCTCCCTGGAGCTGCAGG | |
| | | ACCACAATGAGACCTGCCATGCATGATAATGA | |
| 20 | Membrane | ATGGCAGATTCCAACGGTACTATTACCGTTGAAGAGCTTAAAAAGC | 374 |
| | | TCCTTGAAGAATGGAACCTAGTAATAGGTTTCCTATTCCTTACATGG | |
| | | ATTTGTCTTCTACAATTTGCCTATGCCAACAGGAATAGGTTTTTGTAT | |
| | | ATAATTAAGTTAATTTTCCTCTGGCTGTTATGGCCAGTAACTTTAACT | |
| | | TGTTTTGTGCTTGCTGCTGTTTACAGAATAAATTGGATCACCGGTGG | |
| | | AATTGCTATCGCAATGGCTTGTCTTGTAGGCTTGATGTGGCTCAGC | |
| | | TACTTCATTGCTTCTTTCAGACTGTTTGCGCGTACGCGTTCCATGTG | |
| | | GTCATTTAATCCAGAAACTAACATTCTTCTCAACGTGCCACTCCATG | |
| | | GCACTATTCTGACCAGACCGCTTCTAGAAAGTGAACTCGTAATCGG | |
| | | AGCTGTGATCCTTCGTGGACATCTTCGTATTGCTGGACACCATCTA | |
| | | GGACGCTGTGACATCAAGGACCTGCCTAAAGAAATCACTGTTGCTA | |
| | | CATCACGAACGCTTTCTTATTACAAATTGGGAGCTTCGCAGCGTGTA | |
| | | GCAGGTGACTCAGGTTTTGCTGCATACAGTCGCTACAGGATTGGCA | |
| | | ACTATAAATTAAACACAGACCATTCCAGTAGCAGTGACAATATTGCT | |
| | | TTGCTTGTACAGTAATGATGA | |
| 21 | Membrane | ATGGCTGATTCAAATGGCACCATCACTGTGGAGGAACTGAAGAAGC | 375 |
| | (codon | TCCTTGAGGAGTGGAACCTGGTGATTGGCTTCCTCTTCTTAACCTG | |
| | optimized) | GATCTGCCTGCTGCAGTTTGCTTATGCCAACAGAAATCGCTTCCTG | |
| | | TACATCATCAAGCTCATCTTTCTCTGGCTACTTTGGCCTGTAACTTT | |
| | | GACCTGCTTTGTGCTGGCAGCTGTGTACAGAATCAACTGGATTACT | |
| | | GGAGGAATTGCGATAGCCATGGCCTGTTTAGTGGGGCTCATGTGG | |
| | | CTGAGCTACTTCATCGCTTCCTTCCGGCTGTTTGCCCGAACGAGGA | |
| | | GCATGTGGAGCTTCAACCCAGAAACAAATATCCTGCTTAATGTTCC | |
| | | GCTGCATGGAACCATACTCACCAGGCCTCTATTGGAATCTGAGCTG | |
| | | GTCATTGGAGCTGTCATACTCCGGGGCCATTTGCGAATTGCTGGCC | |
| | | ACCACCTGGGTCGTTGTGACATCAAAGATCTGCCCAAGGAGATCAC | |
| | | AGTGGCCACTTCTAGAACATTGTCTTACTATAAACTTGGGGCGAGT | |
| | | CAGAGGGTTGCAGGTGATTCGGGCTTTGCAGCCTATAGCCGCTATA | |
| | | GGATTGGGAACTACAAACTAAATACAGACCACTCATCCTCCAGTGA | |
| | | CAACATTGCATTGCTTGTCCAATGATAATGA | |
| 22 | Envelope | ATGTACTCATTCGTTTCGGAAGAGATAGGTACGTTAATAGTTAATAG | 376 |
| | | CGTACTTCTTTTTCTTGCTTTCGTGGTATTCTTGCTAGTTACACTAGC | |
| | | CATCCTTACTGCGCTTCGATTGTGTGCGTACTGCTGCAATATTGTTA | |
| | | ACGTGAGTCTTGTAAAACCTTCTTTTTACGTTTACTCTCGTGTTAAAA | |
| | | ATCTGAATTCTTCTAGAGTTCCTGATCTTCTGGTCTAATGATGA | |
| 23 | Envelope | ATGTACAGCTTTGTTAGTGAAGAGATTGGCACTCTAATAGTAAATTC | 377 |
| | (codon | TGTGCTGCTCTTCCTTGCTTTTGTGGTGTTCCTGCTGGTCACCCTG | |
| | optimized) | GCCATCCTCACAGCATTAAGGCTCTGTGCCTACTGCTGCAACATCG | |
| | | TCAATGTGTCTCTGGTGAAACCTTCCTTCTATGTGTATTCACGGGTG | |
| | | AAGAACTTGAACAGCTCCAGAGTTCCAGACTTGCTTGTATGATAATG | |
| | | A | |
| 24 | Nucleo- | ATGTCTGATAATGGACCCCAAAATCAGCGAAATGCACTCCGCATTA | 378 |
| | capsid | CGTTTGGTGGACCCTCAGATTCAACTGGCAGTAACCAGAATGGTGG | |
| | | GGCGCGATCAAAACAACGTCGGCCCCAAGGTTTACCCAATAATACT | |
| | | GCGTCTTGGTTCACCGCTCTCACTCAACATGGCAAGGAAGACCTTA | |
| | | AATTCCCTCGAGGACAAGGCGTTCCAATTAACACCAATAGCAGTCC | |
| | | AGATGACCAAATTGGCTACTACCGAAGAGCTACCAGACGAATTCGT | |
| | | GGTGGTGACGGTAAAATGAAAGATCTCAGTCCAAGATGGTATTTCT | |
| | | ACTACCTAGGAACTGGGCCAGAAGCTGGACTTCCCTATGGTGCTAA | |
| | | CAAAGACGGCATCATATGGGTTGCAACTGAGGGAGCCTTGAATACA | |
| | | CCAAAAGATCACATTGGCACCCGCAATCCTGCTAACAATGCTGCAA | |
| | | TCGTGCTACAACTTCCTCAAGGAACAACATTGCCAAAAGGCTTCTAC | |
| | | GCAGAAGGGAGCAGAGGCGGCAGTCAAGCCTCTTCTCGTTCCTCA | |
| | | TCACGTAGTCGCAACAGTTCAAGAAATTCAACTCCAGGCAGCAGTA | |
| | | AACGAACTTCTCCTGCTAGAATGGCTGGCAATGGCGGTGATGCTGC | |
| | | TCTTGCTTTGCTGCTGCTTGACAGATTGAACCAGCTTGAGAGCAAA | |
| | | ATGTCTGGTAAAGGCCAACAACAACAAGGCCAAACTGTCACTAAGA | |
| | | AATCTGCTGCTGAGGCTTCTAAGAAGCCTCGGCAAAAACGTACTGC | |
| | | CACTAAAGCATACAATGTAACACAAGCTTTCGGCAGACGTGGTCCA | |
| | | GAACAAACCCAAGGAAATTTTGGGGACCAGGAACTAATCAGACAAG | |
| | | GAACTGATTACAAACATTGGCCGCAAATTGCACAATTTGCCCCCAG | |
| | | CGCTTCAGCGTTCTTCGGAATGTCGCGCATTGGCATGGAAGTCACA | |
| | | CCTTCGGGAACGTGGTTGACCTACACAGGTGCCATCAAATTGGATG | |
| | | ACAAAGATCCAAATTTCAAAGATCAAGTCATTTTGCTGAATAAGCAT | |
| | | attgacgcatacaaaacattcccaccaacagagcctaaaaaggaca | |
| | | AAAAGAAGAAGGCTGATGAAACTCAAGCCTTACCGCAGAGACAGAA | |
| | | GAAACAGCAAACTGTGACTCTTCTTCCTGCTGCAGATTTGGATGATT | |
| | | TCTCCAAACAATTGCAACAATCCATGAGCCGTGCTGACTCAACTCA | |
| | | GGCCTAATGATGA | |
| 25 | Nucleo- | ATGTCAGACAATGGGCCCCAGAACCAGAGAAATGCCCTTCGCATCA | 379 |
| | capsid | CGTTTGGTGGCCCTAGTGACAGCACCGGCAGCAATCAGAATGGAG | |
| | (codon | GTGCCCGCAGTAAACAAAGGAGACCACAGGGACTGCCAAACAACA | |
| | optimized) | CAGCATCCTGGTTTACTGCACTTACGCAGCATGGTAAGGAGGACCT | |
| | | GAAGTTCCCAAGAGGACAGGGAGTCCCCATCAACACCAACAGCTC | |
| | | ACCAGATGATCAGATCGGCTATTACCGGCGGGCTACTCGCCGCATA | |
| | | CGAGGGGGCGATGGCAAGATGAAGGATCTAAGTCCGCGTTGGTAC | |
| | | TTCTACTATCTGGGCACAGGGCCTGAAGCTGGCCTGCCTTATGGG | |
| | | GCCAATAAGGATGGCATCATCTGGGTGGCCACTGAGGGGGCTTTG | |
| | | AATACCCCAAAAGACCACATCGGGACTCGAAACCCTGCCAACAATG | |
| | | CAGCCATTGTCCTTCAGCTCCCACAAGGGACAACATTACCCAAGGG | |
| | | CTTCTATGCAGAGGGCTCCAGGGGAGGTTCTCAAGCCAGCAGCCG | |
| | | GAGCAGCTCGCGGTCCCGGAACTCAAGCCGAAATTCCACTCCTGG | |
| | | CAGCTCCAAGAGAACATCACCTGCGAGGATGGCTGGAAATGGAGG | |
| | | CGATGCTGCTCTGGCCCTCCTTTTGCTGGACAGGCTCAACCAGCTG | |
| | | GAGAGTAAAATGAGTGGAAAGGGGCAGCAGCAACAGGGCCAGACT | |
| | | GTGACCAAGAAGTCTGCAGCAGAAGCGTCCAAGAAACCCAGGCAG | |
| | | AAAAGGACAGCCACAAAAGCCTATAATGTGACACAAGCCTTTGGGA | |
| | | GAAGAGGGCCAGAGCAGACCCAGGGAAACTTTGGAGACCAGGAGC | |
| | | TGATTCGTCAAGGTACAGACTACAAGCACTGGCCACAGATTGCTCA | |
| | | GTTTGCTCCCTCTGCATCAGCCTTCTTCGGCATGAGCAGGATAGGT | |
| | | ATGGAGGTGACTCCGAGCGGAACCTGGCTGACCTACACCGGAGCC | |
| | | ATCAAGCTGGATGACAAGGATCCCAACTTCAAGGACCAGGTAATTC | |
| | | TCTTAAACAAGCATATTGATGCCTACAAAACCTTTCCTCCAACAGAA | |
| | | CCCAAAAAGGACAAAAAAAAAAAAGCTGATGAAACTCAGGCCCTAC | |
| | | CTCAGCGGCAAAAGAAACAACAGACGGTTACCCTACTCCCGGCTG | |
| | | CAGATTTGGACGACTTCTCTAAGCAGCTGCAGCAGTCCATGTCCAG | |
| | | AGCTGACTCTACCCAAGCATGATAATGA | |
| 26 | Spike (2- | ATGTTCGTGTTCCTGGTGCTGCTGCCCCTGGTGAGCAGCCAGTGC | 380 |
| | proline | GTGAACCTGATCACCAGGACCCAGAGCTACACCAACAGCTTCACCA | |
| | substitute) | GGGGCGTGTACTACCCCGACAAGGTGTTCAGGAGCAGCGTGCTGC | |
| | | ACAGCACCCAGGACCTGTTCCTGCCCTTCTTCAGCAACGTGACCTG | |
| | | GTTCCACGCCATCAGCGGCACCAACGGCACCAAGAGGTTCGACAA | |
| | | CCCCGTGCTGCCCTTCAACGACGGCGTGTACTTCGCCAGCACCGA | |
| | | GAAGAGCAACATCATCAGGGGCTGGATCTTCGGCACCACCCTGGA | |
| | | CAGCAAGACCCAGAGCCTGCTGATCGTGAACAACGCCACCAACGT | |
| | | GGTGATCAAGGTGTGCGAGTTCCAGTTCTGCAACGACCCCTTCCTG | |
| | | GACGTGTACTACCACAAGAACAACAAGAGCTGGATGGAGAGCGAG | |
| | | TTCAGGGTGTACAGCAGCGCCAACAACTGCACCTTCGAGTACGTGA | |
| | | GCCAGCCCTTCCTGATGGACCTGGAGGGCAAGCAGGGCAACTTCA | |
| | | AGAACCTGAGGGAGTTCGTGTTCAAGAACATCGACGGCTACTTCAA | |
| | | GATCTACAGCAAGCACACCCCCATCAACCTGGGCAGGGACCTGCC | |
| | | CCAGGGCTTCAGCGCCCTGGAGCCCCTGGTGGACCTGCCCATCGG | |
| | | CATCAACATCACCAGGTTCCAGACCCTGCTGGCCCTGCACAGGAG | |
| | | CTACCTGACCCCCGGCGACAGCAGCAGCGGCTGGACCGCCGGCG | |
| | | CCGCCGCCTACTACGTGGGCTACCTGCAGCCCAGGACCTTCCTGC | |
| | | TGAAGTACAACGAGAACGGCACCATCACCGACGCCGTGGACTGCG | |
| | | CCCTGGACCCCCTGAGCGAGACCAAGTGCACCCTGAAGAGCTTCA | |
| | | CCGTGGAGAAGGGCATCTACCAGACCAGCAACTTCAGGGTGCAGC | |
| | | CCACCGAGAGCATCGTGAGGTTCCCCAACATCACCAACCTGTGCC | |
| | | CCTTCGACGAGGTGTTCAACGCCACCAGGTTCGCCAGCGTGTACG | |
| | | CCTGGAACAGGAAGAGGATCAGCAACTGCGTGGCCGACTACAGCG | |
| | | TGCTGTACAACTTCGCCCCCTTCTTCGCCTTCAAGTGCTACGGCGT | |
| | | GAGCCCCACCAAGCTGAACGACCTGTGCTTCACCAACGTGTACGC | |
| | | CGACAGCTTCGTGATCAGGGGCAACGAGGTGAGCCAGATCGCCCC | |
| | | CGGCCAGACCGGCAACATCGCCGACTACAACTACAAGCTGCCCGA | |
| | | CGACTTCACCGGCTGCGTGATCGCCTGGAACAGCAACAAGCTGGA | |
| | | CAGCAAGGTGGGCGGCAACTACAACTACAGGTACAGGCTGTTCAG | |
| | | GAAGAGCAACCTGAAGCCCTTCGAGAGGGACATCAGCACCGAGAT | |
| | | CTACCAGGCCGGCAACAAGCCCTGCAACGGCGTGGCCGGCGTGA | |
| | | ACTGCTACTTCCCCCTGAGGAGCTACGGCTTCAGGCCCACCTACG | |
| | | GCGTGGGCCACCAGCCCTACAGGGTGGTGGTGCTGAGCTTCGAGC | |
| | | TGCTGCACGCCCCCGCCACCGTGTGCGGCCCCAAGAAGAGCACCA | |
| | | ACCTGGTGAAGAACAAGTGCGTGAACTTCAACTTCAACGGCCTGAC | |
| | | CGGCACCGGCGTGCTGACCGAGAGCAACAAGAAGTTCCTGCCCTT | |
| | | CCAGCAGTTCGGCAGGGACATCGCCGACACCACCGACGCCGTGAG | |
| | | GGACCCCCAGACCCTGGAGATCCTGGACATCACCCCCTGCAGCTT | |
| | | CGGCGGCGTGAGCGTGATCACCCCCGGCACCAACACCAGCAACCA | |
| | | GGTGGCCGTGCTGTACCAGGGCGTGAACTGCACCGAGGTGCCCGT | |
| | | GGCCATCCACGCCGACCAGCTGACCCCCACCTGGAGGGTGTACAG | |
| | | CACCGGCAGCAACGTGTTCCAGACCAGGGCCGGCTGCCTGATCGG | |
| | | CGCCGAGTACGTGAACAACAGCTACGAGTGCGACATCCCCATCGG | |
| | | CGCCGGCATCTGCGCCAGCTACCAGACCCAGACCAAGAGCCACCA | |
| | | GCAGGCCCAGAGCGTGGCCAGCCAGAGCATCATCGCCTACACCAT | |
| | | GAGCCTGGGCGCCGAGAACAGCGTGGCCTACAGCAACAACAGCAT | |
| | | CGCCATCCCCACCAACTTCACCATCAGCGTGACCACCGAGATCCTG | |
| | | CCCGTGAGCATGACCAAGACCAGCGTGGACTGCACCATGTACATCT | |
| | | GCGGCGACAGCACCGAGTGCAGCAACCTGCTGCTGCAGTACGGCA | |
| | | GCTTCTGCACCCAGCTGAAGAGGGCCCTGACCGGCATCGCCGTGG | |
| | | AGCAGGACAAGAACACCCAGGAGGTGTTCGCCCAGGTGAAGCAGA | |
| | | TCTACAAGACCCCCCCCATCAAGTACTTCGGCGGCTTCAACTTCAG | |
| | | CCAGATCCTGCCCGACCCCAGCAAGCCCAGCAAGAGGAGCTTCAT | |
| | | CGAGGACCTGCTGTTCAACAAGGTGACCCTGGCCGACGCCGGCTT | |
| | | CATCAAGCAGTACGGCGACTGCCTGGGCGACATCGCCGCCAGGGA | |
| | | CCTGATCTGCGCCCAGAAGTTCAACGGCCTGACCGTGCTGCCCCC | |
| | | CCTGCTGACCGACGAGATGATCGCCCAGTACACCAGCGCCCTGCT | |
| | | GGCCGGCACCATCACCAGCGGCTGGACCTTCGGCGCCGGCGCCG | |
| | | CCCTGCAGATCCCCTTCGCCATGCAGATGGCCTACAGGTTCAACG | |
| | | GCATCGGCGTGACCCAGAACGTGCTGTACGAGAACCAGAAGCTGA | |
| | | TCGCCAACCAGTTCAACAGCGCCATCGGCAAGATCCAGGACAGCC | |
| | | TGAGCAGCACCGCCAGCGCCCTGGGCAAGCTGCAGGACGTGGTG | |
| | | AACCACAACGCCCAGGCCCTGAACACCCTGGTGAAGCAGCTGAGC | |
| | | AGCAAGTTCGGCGCCATCAGCAGCGTGCTGAACGACATCCTGAGC | |
| | | AGGCTGGACCCCCCCGAGGCCGAGGTGCAGATCGACAGGCTGAT | |
| | | CACCGGCAGGCTGCAGAGCCTGCAGACCTACGTGACCCAGCAGCT | |
| | | GATCAGGGCCGCCGAGATCAGGGCCAGCGCCAACCTGGCCGCCA | |
| | | CCAAGATGAGCGAGTGCGTGCTGGGCCAGAGCAAGAGGGTGGACT | |
| | | TCTGCGGCAAGGGCTACCACCTGATGAGCTTCCCCCAGAGCGCCC | |
| | | CCCACGGCGTGGTGTTCCTGCACGTGACCTACGTGCCCGCCCAGG | |
| | | AGAAGAACTTCACCACCGCCCCCGCCATCTGCCACGACGGCAAGG | |
| | | CCCACTTCCCCAGGGAGGGCGTGTTCGTGAGCAACGGCACCCACT | |
| | | GGTTCGTGACCCAGAGGAACTTCTACGAGCCCCAGATCATCACCAC | |
| | | CGACAACACCTTCGTGAGCGGCAACTGCGACGTGGTGATCGGCAT | |
| | | CGTGAACAACACCGTGTACGACCCCCTGCAGCCCGAGCTGGACAG | |
| | | CTTCAAGGAGGAGCTGGACAAGTACTTCAAGAACCACACCAGCCCC | |
| | | GACGTGGACCTGGGCGACATCAGCGGCATCAACGCCAGCGTGGTG | |
| | | AACATCCAGAAGGAGATCGACAGGCTGAACGAGGTGGCCAAGAAC | |
| | | CTGAACGAGAGCCTGATCGACCTGCAGGAGCTGGGCAAGTACGAG | |
| | | CAGTACATCAAGTGGCCCTGGTACATCTGGCTGGGCTTCATCGCCG | |
| | | GCCTGATCGCCATCGTGATGGTGACCATCATGCTGTGCTGCATGAC | |
| | | CAGCTGCTGCAGCTGCCTGAAGGGCTGCTGCAGCTGCGGCAGCTG | |
| | | CTGCAAGTTCGACGAGGACGACAGCGAGCCCGTGCTGAAGGGCGT | |
| | | GAAGCTGCACTACACCTGATGA | |
| 27 | Spike (2- | ATGTTTGTCTTTTTGGTACTCCTCCCTCTTGTCTCGTCTCAGTGTGT | 381 |
| | proline | CAACTTGATTACACGCACTCAAAGTTACACCAACTCCTTTACCCGAG | |
| | substitute) | GAGTCTACTATCCAGATAAAGTGTTCAGATCTAGCGTGTTGCATAGC | |
| | (codon | ACGCAGGATCTCTTTCTCCCGTTTTTCAGCAACGTGACCTGGTTCC | |
| | optimized) | ATGCAATATCTGGCACAAATGGGACCAAGCGGTTTGACAATCCTGT | |
| | | GTTACCGTTCAATGATGGCGTGTACTTCGCCAGTACCGAAAAGTCC | |
| | | AACATCATCAGAGGCTGGATCTTCGGCACAACCCTGGATTCTAAAA | |
| | | CTCAGTCCCTTCTGATAGTCAACAACGCAACCAACGTGGTGATAAA | |
| | | GGTGTGTGAATTCCAGTTTTGTAACGACCCCTTCCTTGACGTTTATT | |
| | | ATCACAAGAATAACAAGAGTTGGATGGAGAGCGAATTTCGGGTATA | |
| | | TTCCTCAGCAAACAACTGCACATTTGAATATGTGAGTCAGCCCTTCC | |
| | | TCATGGACCTCGAAGGAAAGCAAGGCAACTTTAAGAACCTGAGGGA | |
| | | GTTCGTGTTCAAGAACATCGACGGCTATTTAAGATCTACTCTAAGC | |
| | | ACACCCCCATCAATCTTGGCAGAGACCTACCCCAGGGCTTTTCGGC | |
| | | CTTGGAGCCTCTGGTGGATCTCCCCATCGGTATCAACATCACCCGA | |
| | | TTCCAAACTCTCCTGGCACTGCACAGGTCATATCTAACACCCGGGG | |
| | | ACAGCAGCAGTGGGTGGACAGCTGGCGCCGCGGCTTACTATGTTG | |
| | | GCTACCTGCAGCCACGTACCTTCCTGCTGAAATACAATGAAAACGG | |
| | | AACGATTACAGATGCGGTGGACTGTGCACTGGATCCATTGTCTGAA | |
| | | ACTAAATGTACACTAAAATCCTTTACTGTTGAGAAGGGCATCTACCA | |
| | | AACTTCTAATTTCAGGGTTCAGCCCACCGAATCCATCGTTCGCTTCC | |
| | | CCAACATTACTAATCTGTGTCCATTTGATGAAGTGTTTAATGCCACC | |
| | | CGCTTCGCTTCCGTGTATGCATGGAACAGGAAGAGAATCTCAAACT | |
| | | GTGTGGCAGACTATTCTGTTCTGTACAATTTGCTCCTTTCTTTGCC | |
| | | TTCAAGTGCTATGGAGTGTCACCTACAAAGCTCAATGACCTCTGCTT | |
| | | CACAAATGTCTACGCCGATTCTTTTGTGATTCGTGGAAATGAAGTTT | |
| | | CCCAGATCGCACCCGGCCAGACGGGAAACATAGCCGACTACAACT | |
| | | ACAAGCTGCCGGATGACTTCACGGGCTGTGTCATTGCTTGGAATTC | |
| | | AAATAAATTAGATAGTAAAGTGGGGGGGAACTATAATTACCGCTACC | |
| | | GGCTTTTTCGCAAATCAAATTTGAAGCCATTTGAGCGTGATATTTCG | |
| | | ACTGAAATCTATCAGGCTGGGAACAAACCCTGCAACGGGGTAGCA | |
| | | GGAGTCAATTGCTACTTTCCTTTACGCAGCTACGGGTTCCGACCTA | |
| | | CCTATGGTGTGGGCCACCAACCATACAGGGTGGTTGTCTTGTCCTT | |
| | | CGAGCTTCTTCATGCCCCGGCAACGGTGTGCGGTCCTAAAAAGAGT | |
| | | ACCAATCTAGTAAAGAATAAGTGTGTGAACTTTAACTTCAATGGTTT | |
| | | GACCGGCACTGGCGTTTTGACTGAGTCCAACAAGAAGTTCTTACCC | |
| | | TTTCAGCAGTTCGGGCGGGACATCGCCGACACTACAGATGCAGTG | |
| | | CGCGATCCGCAGACACTGGAGATTTTGGACATTACCCCTTGCTCCT | |
| | | TTGGTGGAGTATCTGTAATAACCCCAGGCACGAACACTAGTAATCA | |
| | | AGTCGCCGTGCTGTATCAAGGTGTTAACTGTACCGAGGTGCCTGTG | |
| | | GCTATTCATGCTGACCAGCTCACTCCCACATGGAGAGTCTACTCTA | |
| | | CAGGTAGCAATGTATTTCAAACAAGGGCTGGGTGCCTGATAGGAGC | |
| | | CGAGTACGTGAATAACTCTTATGAGTGTGACATCCCAATCGGAGCT | |
| | | GGGATTTGTGCCTCTTACCAGACCCAGACGAAGTCACACCAGCAG | |
| | | GCTCAATCAGTGGCGTCTCAGAGTATCATCGCCTACACTATGTCCC | |
| | | TCGGAGCAGAAAATTCAGTTGCATACAGCAATAACTCCATCGCCATT | |
| | | CCAACTAACTTCACTATATCGGTGACCACAGAGATCCTGCCAGTCA | |
| | | GCATGACAAAAACATCAGTAGACTGCACCATGTATATATGCGGGGA | |
| | | CAGCACAGAATGTTCTAACCTGCTGTTGCAATATGGGAGCTTCTGC | |
| | | ACTCAGTTGAAGAGGGCTCTGACTGGAATCGCTGTGGAGCAGGAC | |
| | | AAGAATACCCAGGAAGTGTTCGCCCAGGTGAAGCAGATATACAAAA | |
| | | CCCCTCCAATCAAATACTTTGGCGGGTTTAACTTCAGCCAAATTCTG | |
| | | CCCGATCCCTCCAAGCCTAGTAAAAGGTCCTTCATTGAAGATCTGC | |
| | | TGTTCAACAAAGTCACACTGGCAGATGCTGGATTCATCAAGCAGTA | |
| | | TGGTGACTGTCTGGGCGACATCGCAGCGCGGGATCTGATTTGTGC | |
| | | ACAGAAGTTTAATGGGCTTACAGTCTTGCCTCCTCTGCTGACAGAC | |
| | | GAGATGATTGCCCAGTACACGTCTGCCTTGCTCGCTGGTACCATAA | |
| | | CCAGCGGATGGACCTTCGGAGCCGGCGCTGCCCTTCAGATCCCTT | |
| | | TCGCTATGCAGATGGCCTATAGATTTAATGGCATCGGAGTGACTCA | |
| | | GAACGTTCTCTACGAGAATCAGAAACTGATTGCCAACCAGTTTAACA | |
| | | GTGCGATCGGAAAGATTCAAGACAGTCTATCCAGCACGGCTTCTGC | |
| | | ACTGGGTAAACTGCAAGATGTGGTTAACCACAACGCCCAGGCCCTG | |
| | | AACACACTCGTGAAACAGCTCTCTTCCAAGTTCGGTGCCATCAGCT | |
| | | CAGTTCTTAACGATATACTGAGTAGACTAGACCCTCCAGAGGCCGA | |
| | | AGTGCAGATCGACAGGCTTATCACAGGTCGGCTGCAGTCTCTACAA | |
| | | ACCTATGTCACACAACAGTTAATCAGAGCAGCTGAGATCCGAGCTT | |
| | | CCGCGAACCTCGCGGCAACAAAGATGTCTGAGTGCGTCCTGGGGC | |
| | | AGAGCAAGCGGGTAGATTTCTGTGGGAAAGGATACCATCTGATGTC | |
| | | ATTTCCCCAGAGCGCCCCACATGGTGTAGTTTTTCTGCATGTGACC | |
| | | TACGTCCCTGCCCAAGAAAAAAATTTCACTACGGCTCCAGCTATCT | |
| | | GCCACGATGGAAAAGCGCACTTCCCCCGAGAGGGCGTTTTTGTCA | |
| | | GTAATGGTACCCACTGGTTTGTGACACAGAGAAATTTCTATGAGCC | |
| | | ACAGATAATTACTACCGACAACACATTTGTCTCTGGGAATTGTGATG | |
| | | TCGTAATTGGAATTGTGAACAATACAGTATATGATCCATTGCAGCCT | |
| | | GAGCTAGACTCTTTCAAAGAAGAGCTGGATAAATATTTCAAGAACCA | |
| | | TACCTCGCCTGACGTGGACTTGGGGGATATCTCCGGAATAAATGCA | |
| | | TCCGTGGTGAATATCCAGAAGGAAATTGATAGACTGAATGAGGTGG | |
| | | CCAAAAACCTCAATGAGAGTTTGATTGACCTTCAGGAGTTAGGCAA | |
| | | GTATGAACAGTACATTAAGTGGCCATGGTACATCTGGCTGGGTTTC | |
| | | ATTGCCGGCCTCATAGCAATTGTCATGGTGACCATCATGCTCTGCT | |
| | | GCATGACTTCCTGCTGCAGCTGCCTTAAAGGCTGCTGTAGCTGTGG | |
| | | GTCCTGTTGCAAATTTGATGAGGACGACTCAGAGCCGGTGCTGAAG | |
| | | GGAGTTAAATTGCACTACACTTGATAA | |
| 28 | Spike (6 | ATGTTCGTGTTCCTGGTGCTGCTGCCCCTGGTGAGCAGCCAGTGC | 382 |
| | proline | GTGAACCTGATCACCAGGACCCAGAGCTACACCAACAGCTTCACCA | |
| | substitute) | GGGGCGTGTACTACCCCGACAAGGTGTTCAGGAGCAGCGTGCTGC | |
| | | ACAGCACCCAGGACCTGTTCCTGCCCTTCTTCAGCAACGTGACCTG | |
| | | GTTCCACGCCATCAGCGGCACCAACGGCACCAAGAGGTTCGACAA | |
| | | CCCCGTGCTGCCCTTCAACGACGGCGTGTACTTCGCCAGCACCGA | |
| | | GAAGAGCAACATCATCAGGGGCTGGATCTTCGGCACCACCCTGGA | |
| | | CAGCAAGACCCAGAGCCTGCTGATCGTGAACAACGCCACCAACGT | |
| | | GGTGATCAAGGTGTGCGAGTTCCAGTTCTGCAACGACCCCTTCCTG | |
| | | GACGTGTACTACCACAAGAACAACAAGAGCTGGATGGAGAGCGAG | |
| | | TTCAGGGTGTACAGCAGCGCCAACAACTGCACCTTCGAGTACGTGA | |
| | | GCCAGCCCTTCCTGATGGACCTGGAGGGCAAGCAGGGCAACTTCA | |
| | | AGAACCTGAGGGAGTTCGTGTTCAAGAACATCGACGGCTACTTCAA | |
| | | GATCTACAGCAAGCACACCCCCATCAACCTGGGCAGGGACCTGCC | |
| | | CCAGGGCTTCAGCGCCCTGGAGCCCCTGGTGGACCTGCCCATCGG | |
| | | CATCAACATCACCAGGTTCCAGACCCTGCTGGCCCTGCACAGGAG | |
| | | CTACCTGACCCCCGGCGACAGCAGCAGCGGCTGGACCGCCGGCG | |
| | | CCGCCGCCTACTACGTGGGCTACCTGCAGCCCAGGACCTTCCTGC | |
| | | TGAAGTACAACGAGAACGGCACCATCACCGACGCCGTGGACTGCG | |
| | | CCCTGGACCCCCTGAGCGAGACCAAGTGCACCCTGAAGAGCTTCA | |
| | | CCGTGGAGAAGGGCATCTACCAGACCAGCAACTTCAGGGTGCAGC | |
| | | CCACCGAGAGCATCGTGAGGTTCCCCAACATCACCAACCTGTGCC | |
| | | CCTTCGACGAGGTGTTCAACGCCACCAGGTTCGCCAGCGTGTACG | |
| | | CCTGGAACAGGAAGAGGATCAGCAACTGCGTGGCCGACTACAGCG | |
| | | TGCTGTACAACTTCGCCCCCTTCTTCGCCTTCAAGTGCTACGGCGT | |
| | | GAGCCCCACCAAGCTGAACGACCTGTGCTTCACCAACGTGTACGC | |
| | | CGACAGCTTCGTGATCAGGGGCAACGAGGTGAGCCAGATCGCCCC | |
| | | CGGCCAGACCGGCAACATCGCCGACTACAACTACAAGCTGCCCGA | |
| | | CGACTTCACCGGCTGCGTGATCGCCTGGAACAGCAACAAGCTGGA | |
| | | CAGCAAGGTGGGCGGCAACTACAACTACAGGTACAGGCTGTTCAG | |
| | | GAAGAGCAACCTGAAGCCCTTCGAGAGGGACATCAGCACCGAGAT | |
| | | CTACCAGGCCGGCAACAAGCCCTGCAACGGCGTGGCCGGCGTGA | |
| | | ACTGCTACTTCCCCCTGAGGAGCTACGGCTTCAGGCCCACCTACG | |
| | | GCGTGGGCCACCAGCCCTACAGGGTGGTGGTGCTGAGCTTCGAGC | |
| | | TGCTGCACGCCCCCGCCACCGTGTGCGGCCCCAAGAAGAGCACCA | |
| | | ACCTGGTGAAGAACAAGTGCGTGAACTTCAACTTCAACGGCCTGAC | |
| | | CGGCACCGGCGTGCTGACCGAGAGCAACAAGAAGTTCCTGCCCTT | |
| | | CCAGCAGTTCGGCAGGGACATCGCCGACACCACCGACGCCGTGAG | |
| | | GGACCCCCAGACCCTGGAGATCCTGGACATCACCCCCTGCAGCTT | |
| | | CGGCGGCGTGAGCGTGATCACCCCCGGCACCAACACCAGCAACCA | |
| | | GGTGGCCGTGCTGTACCAGGGCGTGAACTGCACCGAGGTGCCCGT | |
| | | GGCCATCCACGCCGACCAGCTGACCCCCACCTGGAGGGTGTACAG | |
| | | CACCGGCAGCAACGTGTTCCAGACCAGGGCCGGCTGCCTGATCGG | |
| | | CGCCGAGTACGTGAACAACAGCTACGAGTGCGACATCCCCATCGG | |
| | | CGCCGGCATCTGCGCCAGCTACCAGACCCAGACCAAGAGCCACCA | |
| | | GCAGGCCCAGAGCGTGGCCAGCCAGAGCATCATCGCCTACACCAT | |
| | | GAGCCTGGGCGCCGAGAACAGCGTGGCCTACAGCAACAACAGCAT | |
| | | CGCCATCCCCACCAACTTCACCATCAGCGTGACCACCGAGATCCTG | |
| | | CCCGTGAGCATGACCAAGACCAGCGTGGACTGCACCATGTACATCT | |
| | | GCGGCGACAGCACCGAGTGCAGCAACCTGCTGCTGCAGTACGGCA | |
| | | GCTTCTGCACCCAGCTGAAGAGGGCCCTGACCGGCATCGCCGTGG | |
| | | AGCAGGACAAGAACACCCAGGAGGTGTTCGCCCAGGTGAAGCAGA | |
| | | TCTACAAGACCCCCCCCATCAAGTACTTCGGCGGCTTCAACTTCAG | |
| | | CCAGATCCTGCCCGACCCCAGCAAGCCCAGCAAGAGGAGCCCCAT | |
| | | CGAGGACCTGCTGTTCAACAAGGTGACCCTGGCCGACGCCGGCTT | |
| | | CATCAAGCAGTACGGCGACTGCCTGGGCGACATCGCCGCCAGGGA | |
| | | CCTGATCTGCGCCCAGAAGTTCAACGGCCTGACCGTGCTGCCCCC | |
| | | CCTGCTGACCGACGAGATGATCGCCCAGTACACCAGCGCCCTGCT | |
| | | GGCCGGCACCATCACCAGCGGCTGGACCTTCGGCGCCGGCCCCG | |
| | | CCCTGCAGATCCCCTTCCCCATGCAGATGGCCTACAGGTTCAACGG | |
| | | CATCGGCGTGACCCAGAACGTGCTGTACGAGAACCAGAAGCTGAT | |
| | | CGCCAACCAGTTCAACAGCGCCATCGGCAAGATCCAGGACAGCCT | |
| | | GAGCAGCACCCCCAGCGCCCTGGGCAAGCTGCAGGACGTGGTGA | |
| | | ACCACAACGCCCAGGCCCTGAACACCCTGGTGAAGCAGCTGAGCA | |
| | | GCAAGTTCGGCGCCATCAGCAGCGTGCTGAACGACATCCTGAGCA | |
| | | GGCTGGACCCCCCCGAGGCCGAGGTGCAGATCGACAGGCTGATC | |
| | | ACCGGCAGGCTGCAGAGCCTGCAGACCTACGTGACCCAGCAGCTG | |
| | | ATCAGGGCCGCCGAGATCAGGGCCAGCGCCAACCTGGCCGCCAC | |
| | | CAAGATGAGCGAGTGCGTGCTGGGCCAGAGCAAGAGGGTGGACTT | |
| | | CTGCGGCAAGGGCTACCACCTGATGAGCTTCCCCCAGAGCGCCCC | |
| | | CCACGGCGTGGTGTTCCTGCACGTGACCTACGTGCCCGCCCAGGA | |
| | | GAAGAACTTCACCACCGCCCCCGCCATCTGCCACGACGGCAAGGC | |
| | | CCACTTCCCCAGGGAGGGCGTGTTCGTGAGCAACGGCACCCACTG | |
| | | GTTCGTGACCCAGAGGAACTTCTACGAGCCCCAGATCATCACCACC | |
| | | GACAACACCTTCGTGAGCGGCAACTGCGACGTGGTGATCGGCATC | |
| | | GTGAACAACACCGTGTACGACCCCCTGCAGCCCGAGCTGGACAGC | |
| | | TTCAAGGAGGAGCTGGACAAGTACTTCAAGAACCACACCAGCCCC | |
| | | GACGTGGACCTGGGCGACATCAGCGGCATCAACGCCAGCGTGGTG | |
| | | AACATCCAGAAGGAGATCGACAGGCTGAACGAGGTGGCCAAGAAC | |
| | | CTGAACGAGAGCCTGATCGACCTGCAGGAGCTGGGCAAGTACGAG | |
| | | CAGTACATCAAGTGGCCCTGGTACATCTGGCTGGGCTTCATCGCCG | |
| | | GCCTGATCGCCATCGTGATGGTGACCATCATGCTGTGCTGCATGAC | |
| | | CAGCTGCTGCAGCTGCCTGAAGGGCTGCTGCAGCTGCGGCAGCTG | |
| | | CTGCAAGTTCGACGAGGACGACAGCGAGCCCGTGCTGAAGGGCGT | |
| | | GAAGCTGCACTACACCTGATGA | |
| 29 | Spike (6 | ATGTTTGTATTCCTGGTACTGCTGCCCCTGGTCAGTAGTCAGTGTG | 383 |
| | proline | TCAATCTGATCACTCGCACGCAAAGTTATACCAATTCCTTCACTCGC | |
| | substitute) | GGTGTCTACTACCCAGACAAGGTGTTTAGGTCCAGCGTGCTCCACA | |
| | (codon | GCACACAAGACTTGTTCTTACCATTTTTCAGTAATGTTACCTGGTTT | |
| | optimized) | CATGCCATCTCCGGCACAAATGGGACCAAGAGGTTCGACAATCCTG | |
| | | TGCTACCCTTCAACGACGGGGTGTACTTTGCTTCCACTGAGAAATC | |
| | | TAATATCATTAGAGGCTGGATCTTTGGTACCACATTAGATTCTAAAA | |
| | | CTCAGTCACTGCTAATTGTGAATAACGCCACGAACGTCGTGATCAA | |
| | | GGTCTGTGAGTTTCAATTCTGCAACGACCCTTTCCTGGATGTCTATT | |
| | | ATCACAAGAACAATAAGAGCTGGATGGAGTCTGAGTTCCGAGTGTA | |
| | | CAGTTCTGCAAACAATTGCACCTTTGAGTACGTAAGTCAGCCTTTTC | |
| | | TCATGGATCTGGAAGGCAAGCAGGGTAATTTCAAGAATCTGCGGGA | |
| | | ATTCGTGTTCAAGAACATTGACGGATATTTCAAAATATATTCAAAGC | |
| | | ATACACCGATTAACTTGGGGCGGGATCTGCCACAAGGGTTTAGTGC | |
| | | CCTGGAGCCCTTGGTGGACTTGCCCATTGGCATCAACATTACCCGT | |
| | | TTTCAGACCCTTCTGGCATTGCACAGGTCTTATCTCACACCTGGGG | |
| | | ATTCCAGCAGTGGCTGGACAGCCGGTGCAGCTGCCTACTACGTGG | |
| | | GCTACCTTCAGCCACGTACATTCCTCCTAAAATATAACGAAAACGG | |
| | | GACCATTACTGATGCAGTCGACTGTGCACTGGACCCACTGTCCGAG | |
| | | ACTAAATGCACATTAAAGAGCTTCACTGTGGAAAAGGGGATTTACCA | |
| | | GACGTCCAACTTCAGGGTTCAGCCCACCGAAAGCATCGTCCGCTTC | |
| | | CCAAATATCACCAACCTGTGTCCCTTTGATGAGGTGTTCAACGCCA | |
| | | CACGTTTTGCTAGCGTCTATGCCTGGAACCGGAAAAGAATCTCGAA | |
| | | CTGTGTGGCAGACTACTCAGTGCTCTACAACTTTGCTCCTTTTTTCG | |
| | | CCTTCAAATGTTACGGCGTTTCACCTACGAAACTGAATGACCTCTGC | |
| | | TTTACCAATGTCTATGCTGATTCATTTGTGATCCGCGGCAATGAGGT | |
| | | CAGTCAAATAGCACCCGGGCAGACTGGGAACATAGCCGATTACAAT | |
| | | TACAAACTACCGGATGACTTCACAGGCTGCGTTATTGCATGGAATT | |
| | | CGAATAAGTTGGACTCAAAGGTTGGAGGAAATTATAACTACCGGTA | |
| | | CAGACTGTTCCGTAAGTCTAATCTGAAGCCCTTCGAACGGGACATT | |
| | | TCCACAGAGATCTACCAGGCTGGAAACAAGCCATGCAATGGCGTG | |
| | | GCAGGTGTGAACTGCTACTTCCCTCTGCGCTCGTATGGGTTTAGAC | |
| | | CCACGTATGGAGTGGGACATCAGCCTTATAGAGTTGTGGTGTTAAG | |
| | | CTTTGAACTGCTTCATGCCCCAGCCACCGTGTGTGGGCCAAAGAAG | |
| | | TCTACTAACCTTGTTAAAAACAAGTGTGTGAATTTTAATTTTAATGGA | |
| | | CTGACCGGAACTGGCGTCCTGACTGAGAGCAACAAGAAATTTCTCC | |
| | | CCTTCCAGCAGTTTGGAAGGGATATTGCTGACACAACCGATGCGGT | |
| | | CCGCGACCCGCAGACGCTTGAAATTCTGGACATCACCCCCTGCTCT | |
| | | TTCGGTGGTGTGTCTGTCATCACGCCTGGCACTAACACATCCAACC | |
| | | AAGTGGCTGTCCTCTATCAGGGAGTTAACTGTACCGAGGTTCCTGT | |
| | | GGCCATCCACGCGGACCAGCTGACTCCAACATGGCGCGTTTACAG | |
| | | CACTGGCTCAAACGTATTCCAGACTCGGGCCGGTTGTTTAATTGGT | |
| | | GCCGAATATGTTAACAACAGTTACGAATGTGATATCCCCATTGGCG | |
| | | CGGGAATCTGTGCGAGCTACCAAACACAGACTAAGTCCCACCAGCA | |
| | | GGCCCAGAGTGTGGCTAGCCAGAGCATCATTGCATACACCATGTCC | |
| | | CTCGGCGCTGAAAATAGTGTTGCTTATTCGAATAATTCCATTGCAAT | |
| | | TCCTACTAACTTCACCATAAGCGTCACGACAGAAATCCTGCCTGTGT | |
| | | CTATGACAAAAACATCCGTCGACTGCACGATGTACATCTGCGGCGA | |
| | | CAGCACAGAGTGCTCAAACCTCCTTCTGCAGTACGGATCTTTCTGT | |
| | | ACACAACTCAAGAGGGCTCTGACAGGGATCGCAGTGGAACAAGAT | |
| | | AAGAATACACAGGAAGTTTTCGCGCAGGTGAAACAGATCTACAAGA | |
| | | CCCCCCCTATTAAGTATTTCGGCGGGTTCAACTTTAGCCAGATTTTG | |
| | | CCAGATCCGAGCAAACCTAGTAAACGATCCCCTATTGAGGACCTGC | |
| | | TCTTTAACAAAGTGACATTGGCAGATGCTGGATTTATCAAACAGTAC | |
| | | GGAGACTGCCTGGGAGACATCGCAGCTCGGGATCTTATCTGTGCA | |
| | | CAGAAGTTCAATGGTCTTACAGTGCTTCCTCCACTTCTGACAGACG | |
| | | AGATGATCGCTCAGTATACAAGTGCTCTCCTTGCGGGCACAATAAC | |
| | | TAGCGGATGGACGTTTGGGGCTGGACCTGCGCTACAGATCCCATTT | |
| | | CCAATGCAGATGGCCTACAGATTCAACGGGATAGGTGTTACTCAGA | |
| | | ATGTCCTCTATGAGAACCAGAAATTAATTGCCAACCAGTTCAACTCC | |
| | | GCAATCGGGAAGATCCAGGATTCACTCTCCAGCACCCCTTCTGCCC | |
| | | TTGGTAAATTACAAGATGTGGTTAATCATAACGCCCAAGCCCTCAAT | |
| | | ACACTAGTGAAGCAACTCAGTAGTAAGTTTGGAGCAATATCATCTGT | |
| | | GCTAAATGATATCTTGTCTCGGTTGGACCCACCTGAGGCAGAAGTG | |
| | | CAGATAGACAGGCTCATCACTGGGAGGTTACAATCACTTCAGACCT | |
| | | ATGTCACTCAACAGCTCATCCGAGCTGCCGAGATTAGAGCTAGCGC | |
| | | CAACCTCGCTGCCACCAAAATGTCTGAATGTGTGTTGGGGCAGTCT | |
| | | AAACGAGTTGACTTTTGTGGCAAGGGCTACCACCTGATGAGCTTTC | |
| | | CCCAGTCAGCACCACATGGGGTAGTTTTTTTGCATGTCACCTACGT | |
| | | ACCCGCCCAGGAGAAGAATTTCACGACCGCTCCCGCTATATGCCAT | |
| | | GACGGCAAGGCCCACTTTCCAAGAGAGGGAGTGTTCGTTAGCAAC | |
| | | GGAACCCACTGGTTCGTAACTCAGAGAAACTTCTACGAACCACAGA | |
| | | TCATCACTACCGATAACACCTTCGTGTCTGGAAACTGTGACGTGGT | |
| | | GATTGGTATAGTCAACAACACAGTATATGATCCCCTCCAACCGGAG | |
| | | CTCGATTCATTTAAGGAGGAGCTGGACAAGTACTTTAAAAATCACAC | |
| | | TTCGCCGGACGTCGACCTTGGTGATATTTCAGGGATCAATGCCTCC | |
| | | GTAGTAAACATACAGAAGGAAATTGACCGATTGAATGAGGTAGCGA | |
| | | AAAACCTGAATGAGTCCCTGATAGATCTCCAGGAGCTTGGCAAATA | |
| | | TGAACAGTATATTAAGTGGCCGTGGTACATCTGGCTGGGCTTCATC | |
| | | GCCGGCCTGATAGCCATTGTGATGGTGACAATCATGCTGTGCTGCA | |
| | | TGACCTCTTGCTGCAGCTGCCTGAAGGGCTGCTGTAGTTGTGGGTC | |
| | | CTGTTGCAAGTTTGATGAGGATGATAGCGAGCCAGTCCTGAAAGGA | |
| | | GTGAAGCTGCACTATACTTGATAA | |

In some embodiments, the large sequences are not limited to the above-mentioned conserved fragments.

In certain embodiments, the large sequence comprises spike glycoprotein (S) or a portion thereof (e.g., the RBD), nucleoprotein or a portion thereof, membrane protein or a portion thereof, and/or ORF1a/b or a portion thereof (see Table 9, SEQ ID NO: 139). In certain embodiments, the large sequence comprises Spike glycoprotein (S) or a portion thereof (e.g., the RBD), Nucleoprotein or a portion thereof, and ORF1a/b or a portion thereof. In further embodiments, the large sequence comprises Spike glycoprotein (S) or a portion thereof (e.g., the RBD), and Nucleocapsid protein or a portion thereof (see Table. 9, SEQ ID NO: 140).

As will be discussed herein, in certain embodiments, the vaccine composition comprises whole spike protein, one or more coronavirus CD4+ T cell target epitopes; and one or more coronavirus CD8+ T cell target epitopes. In certain embodiments, the vaccine composition comprises at least a portion of the spike protein (e.g., wherein the portion comprises a trimerized SARS-CoV-2 receptor-binding domain (RBD)), one or more coronavirus CD4+ T cell target epitopes; and one or more coronavirus CD8+ T cell target epitopes. In some embodiments, the one or more coronavirus CD4+ T cell target epitopes; and one or more coronavirus CD8+ T cell target epitopes are in the form of a large sequence.

In some embodiments, the large sequence(s) are derived from a full-length spike glycoprotein. In other embodiments, the large sequence(s) are derived from a portion of the spike glycoprotein. In some embodiments, the transmembrane anchor of the spike protein has an intact S1-S2 cleavage site. In some embodiments, the spike protein is in its stabilized conformation. In some embodiments, the spike protein is stabilized with proline substitutions at amino acid positions 986 and 987 at the top of the central helix in the S2 subunit. In some embodiments, the composition comprises a SARS-CoV-2 receptor-binding domain (RBD). In some embodiments, the composition comprises a trimerized SARS-CoV-2 receptor-binding domain (RBD). In some embodiments, the trimerized SARS-CoV-2 receptor-binding domain (RBD) sequence is modified by the addition of a T4 fibritin-derived foldon trimerization domain. In some embodiments, the addition of a T4 fibritin-derived foldon trimerization domain increases immunogenicity by multivalent display.

In some embodiments, the spike protein comprises Tyr-489 and Asn-487 (e.g., Tyr-489 and Asn-487 help with interaction with Tyr 83 and Gln-24 on ACE-2). In some embodiments, the spike protein comprises Gln-493 (e.g., Gln-493 helps with interaction with Glu-35 and Lys-31 on ACE-2). In some embodiments, the spike protein comprises Tyr-505 (e.g., Tyr-505 helps with interaction with Glu-37 and Arg-393 on ACE-2). In some embodiments, the composition comprises a mutation 682-RRAR-685→682-QQAQ-685 in the S1-S2 cleavage site.

In some embodiments, the spike protein comprising the large sequence(s) comprises at least one proline substitution. In some embodiments, the spike protein comprising the large sequence(s) comprises at least two proline substitutions. For example, the proline substitution may be at position K986 and V987.

Non-limiting examples of Spike protein sequences are disclosed in Table 2.

TABLE 2

| | | SEQ ID |
|---|---|---|
| | Sequence: | NO: |
| SARS-CoV-like | SQCVNLTTRTQLPPAYTNSFTRGVYYPDKVFRSSVLHSTQDLFLPF | 186 |
| Spike-S1-NTD | FSNVTWFHAIHVSGTNGTKRFDNPVLPFNDGVYFASTEKSNIIRGW | |
| 13 bp-304 bp | FGTTLDSKTQSLLIVNNATNVVIKVCEFQFCNDPFLGVYYHKNNKSW | |
| | MESEFRVYSSANNCTFEYVSQPFLMDLEGKQGNFKNLREFVFKNID | |
| | GYFKIYSKHTPINLVRDLPQGFSALEPLVDLPIGINITRFQTLLALHR | |
| | SYLTPGDSSSGWTAGAAAYYVGYLQPRTFLLKYNENGTITDAVDCAL | |
| | DPLSETKCTLK | |
| SARS-CoV-2 | RVQPTESIVRFPNITNLCPFGEVFNATRFASVYAWNRKRISNCVADY | 187 |
| Spike-S-RBD | SVLYNSASFSTFKCYGVSPTKLNDLCFTNVYADSFVIRGDEVRQIAP | |
| 319 bp-541 bp | GQTGKIADYNYKLPDDFTGCVIAWNSNNLDSKVGGNYNYLYRLFRK | |
| | SNLKPFERDISTEIYQAGSTPCNGVEGFNCYFPLQSYGFQPTNGVG | |
| | YQPYRVWVLSFELLHAPATVCGPKKSTNLVKNKCVNF | |
| CoV Spike S1- | FNGLTGTGVLTESNKKFLPFQQFGRDIADTTDAVTRAGCLIGAEHVN | 188 |
| S2_S2 | NSYECDIPIGAGICASYQTQTNRDPQTLEILDITPCSFGGVSVITPGT | |
| 543 bp-1, 208 bp | NTSNQVAVLYQDVNCTEVPVAIHADQLTPTWRVYSTGSNVFQSPR | |
| | RARSVASQSIIAYTMSLGAENSVAYSNNSIAIPTNFTISVTTEILPVSM | |
| | TKTSVDCTMYICGDSTECSNLLLQYGSFCTQLNRALTGIAVEQDKNT | |
| | QEVFAQVKQIYKTPPIKDFGGFNFSQILPDPSKPSKRSFIEDLLFNKV | |
| | TLADAGFIKQYGDCLGDIAARDLICAQKFNGLTVLPPLLTDEMIAQYT | |
| | SALLAGTITSGWTFGAGAALQIPFAMQMAYRFNGIGVTQNVLYENQ | |
| | KLIANQFNSAIGKIQDSLSSTASALGKLQDWNQNAQALNTLVKQLS | |
| | SNFGAISSVLNDILSRLDKVEAEVQIDRLITGRLQSLQTYVTQQLIRAA | |
| | EIRASANLAATKMSECVLGQSKRVDFCGKGYHLMSFPQSAPHGW | |
| | FLHVTYVPAQEKNFTTAPAICHDGKAHFPREGVFVSNGTHWFVTQR | |
| | NFYEPQIITTDNTFVSGNCDWIGIVNNTVYDPLQPELDSFKEELDKY | |
| | FKNHTSPDVDLGDISGINASWNIQKEIDRLNEVAKNLNESLIDLQEL | |
| | GKYEQ | |
| spike glycoprotein | MFVFLVLLPLVSSQCVNLTTRTQLPPAYTNSFTRGVYYPDKVFRSS | 189 |
| with a mutation | VLHSTQDLFLPFFSNVTWFHAIHVSGTNGTKRFDNPVLPFNDGVYF | |
| 682-RRAR-685- | ASTEKSNIIRGWIFGTTLDSKTQSLLIVNNATNWIKVCEFQFCNDPF | |
| 682-QQAQ-685 in | LGVYYHKNNKSWMESEFRVYSSANNCTFEYVSQPFLMDLEGKQG | |
| the S1-S2 | NFKNLREFVFKNIDGYFKIYSKHTPINLVRDLPQGFSALEPLVDLPIGI | |
| cleavage site | NITRFQTLLALHRSYLTPGDSSSGWTAGAAAYYVGYLQPRTFLLKY | |
| | NENGTITDAVDCALDPLSETKCTLKSFTVEKGIYQTSNFRVQPTESIV | |
| | RFPNITNLCPFGEVFNATRFASVYAWNRKRISNCVADYSVLYNSAS | |
| | FSTFKCYGVSPTKLNDLCFTNVYADSFVIRGDEVRQIAPGQTGKIAD | |
| | YNYKLPDDFTGCVIAWNSNNLDSKVGGNYNYLYRLFRKSNLKPFER | |
| | DISTEIYQAGSTPCNGVEGFNCYFPLQSYGFQPTNGVGYQPYRVV | |
| | LSFELLHAPATVCGPKKSTNLVKNKCVNFNFNGLTGTGVLTESNKK | |
| | FLPFQQFGRDIADTTDAVTRAGCLIGAEHVNNSYECDIPIGAGICASY | |
| | QTQTNRDPQTLEILDITPCSFGGVSVITPGTNTSNQVAVLYQDVNCT | |
| | EVPVAIHADQLTPTWRVYSTGSNVFQSPQQAQSVASQSIIAYTMSL | |
| | GAENSVAYSNNSIAIPTNFTISVTTEILPVSMTKTSVDCTMYICGDST | |
| | ECSNLLLQYGSFCTQLNRALTGIAVEQDKNTQEVFAQVKQIYKTPPI | |
| | KDFGGFNFSQILPDPSKPSKRSFIEDLLFNKVTLADAGFIKQYGDCL | |
| | GDIAARDLICAQKFNGLTVLPPLLTDEMIAQYTSALLAGTITSGWTFG | |
| | AGAALQIPFAMQMAYRFNGIGVTQNVLYENQKLIANQFNSAIGKIQD | |
| | SLSSTASALGKLQDVVNQNAQALNTLVKQLSSNFGAISSVLNDILSR | |
| | LDKVEAEVQIDRLITGRLQSLQTYVTOQLIRAAEIRASANLAATKMSE | |
| | CVLGQSKRVDFCGKGYHLMSFPQSAPHGVVFLHVTYVPAQEKNFT | |
| | TAPAICHDGKAHFPREGVFVSNGTHWFVTQRNFYEPQIITTDNTFVS | |
| | GNCDWIGIVNNTVYDPLQPELDSFKEELDKYFKNHTSPDVDLGDIS | |
| | GINASVNIQKEIDRLNEVAKNLNESLIDLQELGKYEQYIKWPWIWL | |
| | GFIAGLIAIVMVTIMLCCMTSCCSCLKGCCSCGSCCKFDEDDSEPVL | |
| | KGVKLHYT | |
| spike glycoprotein | MFVFLVLLPLVSSQCVNLTTRTQLPPAYTNSFTRGVYYPDKVFRSS | 190 |
| with two proline | VLHSTQDLFLPFFSNVTWFHAIHVSGTNGTKRFDNPVLPFNDGVYF | |
| substitutions | ASTEKSNIIRGWIFGTTLDSKTQSLLIVNNATNWIKVCEFQFCNDPF | |
| (K986P, V987P) | LGVYYHKNNKSWMESEFRVYSSANNCTFEYVSQPFLMDLEGKQG | |
| | NFKNLREFVFKNIDGYFKIYSKHTPINLVRDLPQGFSALEPLVDLPIGI | |
| | NITRFQTLLALHRSYLTPGDSSSGWTAGAAAYYVGYLQPRTFLLKY | |
| | NENGTITDAVDCALDPLSETKCTLKSFTVEKGIYQTSNFRVQPTESIV | |
| | RFPNITNLCPFGEVFNATRFASVYAWNRKRISNCVADYSVLYNSAS | |
| | FSTFKCYGVSPTKLNDLCFTNVYADSFVIRGDEVRQIAPGQTGKIAD | |
| | YNYKLPDDFTGCVIAWNSNNLDSKVGGNYNYLYRLFRKSNLKPFER | |
| | DISTEIYQAGSTPCNGVEGFNCYFPLQSYGFQPTNGVGYQPYRWV | |
| | LSFELLHAPATVCGPKKSTNLVKNKCVNFNFNGLTGTGVLTESNKK | |
| | FLPFQQFGRDIADTTDAVRDPQTLEILDITPCSFGGVSVITPGTNTSN | |
| | QVAVLYQDVNCTEVPVAIHADQLTPTWRVYSTGSNVFQTRAGCLIG | |
| | AEHVNNSYECDIPIGAGICASYQTQTNSPRRARSVASQSIIAYTMSL | |
| | GAENSVAYSNNSIAIPTNFTISVTTEILPVSMTKTSVDCTMYICGDST | |
| | ECSNLLLQYGSFCTQLNRALTGIAVEQDKNTQEVFAQVKQIYKTPPI | |
| | KDFGGFNFSQILPDPSKPSKRSFIEDLLFNKVTLADAGFIKQYGDCL | |
| | GDIAARDLICAQKFNGLTVLPPLLTDEMIAQYTSALLAGTITSGWTFG | |
| | AGAALQIPFAMQMAYRFNGIGVTQNVLYENQKLIANQFNSAIGKIQD | |
| | SLSSTASALGKLQDVVNQNAQALNTLVKQLSSNFGAISSVLNDILSR | |
| | LDPPEAEVQIDRLITGRLQSLQTYVTQQLIRAAEIRASANLAATKMSE | |
| | CVLGQSKRVDFCGKGYHLMSFPQSAPHGVVFLHVTYVPAQEKNFT | |
| | TAPAICHDGKAHFPREGVFVSNGTHWFVTQRNFYEPQIITTDNTFVS | |
| | GNCDWIGIVNNTVYDPLQPELDSFKEELDKYFKNHTSPDVDLGDIS | |
| | GINASVNIQKEIDRLNEVAKNLNESLIDLQELGKYEQYIKWPWYIWL | |
| | GFIAGLIAIVMVTIMLCCMTSCCSCLKGCCSCGSCCKFDEDDSEPVL | |
| | KGVKLHYT | |
| spike glycoprotein | MFVFLVLLPLVSSQCVNLTTRTQLPPAYTNSFTRGVYYPDKVFRSS | 191 |
| with four proline | VLHSTQDLFLPFFSNVTWFHAIHVSGTNGTKRFDNPVLPFNDGVYF | |
| substitutions | ASTEKSNIIRGWIFGTTLDSKTQSLLIVNNATNVVIKVCEFQFCNDPF | |
| (F817P, A892P, | LGVYYHKNNKSWMESEFRVYSSANNCTFEYVSQPFLMDLEGKQG | |
| A899P, A942P) | NFKNLREFVFKNIDGYFKIYSKHTPINLVRDLPQGFSALEPLVDLPIGI | |
| | NITRFQTLLALHRSYLTPGDSSSGWTAGAAAYYVGYLQPRTFLLKY | |
| | NENGTITDAVDCALDPLSETKCTLKSFTVEKGIYQTSNFRVQPTESIV | |
| | RFPNITNLCPFGEVFNATRFASVYAWNRKRISNCVADYSVLYNSAS | |
| | FSTFKCYGVSPTKLNDLCFTNVYADSFVIRGDEVRQIAPGQTGKIAD | |
| | YNYKLPDDFTGCVIAWNSNNLDSKVGGNYNYLYRLFRKSNLKPFER | |
| | DISTEIYQAGSTPCNGVEGFNCYFPLQSYGFQPTNGVGYQPYRV | |
| | LSFELLHAPATVCGPKKSTNLVKNKCVNFNFNGLTGTGVLTESNKK | |
| | FLPFQQFGRDIADTTDAVTRAGCLIGAEHVNNSYECDIPIGAGICASY | |
| | QTQTNRDPQTLEILDITPCSFGGVSVITPGTNTSNQVAVLYQDVNCT | |
| | EVPVAIHADQLTPTWRVYSTGSNVFQSPRRARSVASQSIIAYTMSL | |
| | GAENSVAYSNNSIAIPTNFTISVTTEILPVSMTKTSVDCTMYICGDST | |
| | ECSNLLLQYGSFCTQLNRALTGIAVEQDKNTQEVFAQVKQIYKTPPI | |
| | KDFGGFNFSQILPDPSKPSKRSPIEDLLFNKVTLADAGFIKQYGDCL | |
| | GDIAARDLICAQKFNGLTVLPPLLTDEMIAQYTSALLAGTITSGWTFG | |
| | AGPALQIPFPMQMAYRFNGIGVTQNVLYENQKLIANQFNSAIGKIQD | |
| | SLSSTPSALGKLQDVVNQNAQALNTLVKQLSSNFGAISSVLNDILSR | |
| | LDKVEAEVQIDRLITGRLQSLQTYVTQQLIRAAEIRASANLAATKMSE | |
| | CVLGQSKRVDFCGKGYHLMSFPQSAPHGVVFLHVTYVPAQEKNFT | |
| | TAPAICHDGKAHFPREGVFVSNGTHWFVTQRNFYEPQIITTDNTFVS | |
| | GNCDWIGIVNNTVYDPLQPELDSFKEELDKYFKNHTSPDVDLGDIS | |
| | GINASVNIQKEIDRLNEVAKNLNESLIDLQELGKYEQYIKWPWYIWL | |
| | GFIAGLIAIVMVTIMLCCMTSCCSCLKGCCSCGSCCKFDEDDSEPVL | |
| | KGVKLHYT | |
| spike glycoprotein | MFVFLVLLPLVSSQCVNLTTRTQLPPAYTNSFTRGVYYPDKVFRSS | 192 |
| with six proline | VLHSTQDLFLPFFSNVTWFHAIHVSGTNGTKRFDNPVLPFNDGVYF | |
| substitutions | ASTEKSNIIRGWIFGTTLDSKTQSLLIVNNATNWIKVCEFQFCNDPF | |
| (F817P, A892P, | LGVYYHKNNKSWMESEFRVYSSANNCTFEYVSQPFLMDLEGKQG | |
| A899P, A942P, | NFKNLREFVFKNIDGYFKIYSKHTPINLVRDLPQGFSALEPLVDLPIGI | |
| K986P, V987P) | NITRFQTLLALHRSYLTPGDSSSGWTAGAAAYYVGYLQPRTFLLKY | |
| | NENGTITDAVDCALDPLSETKCTLKSFTVEKGIYQTSNFRVQPTESIV | |
| | RFPNITNLCPFGEVFNATRFASVYAWNRKRISNCVADYSVLYNSAS | |
| | FSTFKCYGVSPTKLNDLCFTNVYADSFVIRGDEVRQIAPGQTGKIAD | |
| | YNYKLPDDFTGCVIAWNSNNLDSKVGGNYNYLYRLFRKSNLKPFER | |
| | DISTEIYQAGSTPCNGVEGFNCYFPLQSYGFQPTNGVGYQPYRWV | |
| | LSFELLHAPATVCGPKKSTNLVKNKCVNFNFNGLTGTGVLTESNKK | |
| | FLPFQQFGRDIADTTDAVTRAGCLIGAEHVNNSYECDIPIGAGICASY | |
| | QTQTNRDPQTLEILDITPCSFGGVSVITPGTNTSNQVAVLYQDVNCT | |
| | EVPVAIHADQLTPTWRVYSTGSNVFQSPRRARSVASQSIIAYTMSL | |
| | GAENSVAYSNNSIAIPTNFTISVTTEILPVSMTKTSVDCTMYICGDST | |
| | ECSNLLLQYGSFCTQLNRALTGIAVEQDKNTQEVFAQVKQIYKTPPI | |
| | KDFGGFNFSQILPDPSKPSKRSPIEDLLFNKVTLADAGFIKQYGDCL | |
| | GDIAARDLICAQKFNGLTVLPPLLTDEMIAQYTSALLAGTITSGWTFG | |
| | AGPALQIPFPMQMAYRFNGIGVTQNVLYENQKLIANQFNSAIGKIQD | |
| | SLSSTPSALGKLQDVVNQNAQALNTLVKQLSSNFGAISSVLNDILSR | |
| | LDPPEAEVQIDRLITGRLQSLQTYVTQQLIRAAEIRASANLAATKMSE | |
| | CVLGQSKRVDFCGKGYHLMSFPQSAPHGVVFLHVTYVPAQEKNFT | |
| | TAPAICHDGKAHFPREGVFVSNGTHWFVTQRNFYEPQIITTDNTFVS | |
| | GNCDWIGIVNNTVYDPLQPELDSFKEELDKYFKNHTSPDVDLGDIS | |
| | GINASWNIQKEIDRLNEVAKNLNESLIDLQELGKYEQYIKWPWYIWL | |
| | GFIAGLIAIVMVTIMLCCMTSCCSCLKGCCSCGSCCKFDEDDSEPVL | |
| | KGVKLHYT | |
| spike glycoprotein | MFVFLVLLPLVSSQCVNLTTRTQLPPAYTNSFTRGVYYPDKVFRSS | 193 |
| with six proline | VLHSTQDLFLPFFSNVTWFHAIHVSGTNGTKRFDNPVLPFNDGVYF | |
| substitutions | ASTEKSNIIRGWIFGTTLDSKTQSLLIVNNATNWIKVCEFQFCNDPF | |
| (F817P, A892P, | LGVYYHKNNKSWMESEFRVYSSANNCTFEYVSQPFLMDLEGKQG | |
| A899P, A942P, | NFKNLREFVFKNIDGYFKIYSKHTPINLVRDLPQGFSALEPLVDLPIGI | |
| K986P, V987P) | NITRFQTLLALHRSYLTPGDSSSGWTAGAAAYYVGYLQPRTFLLKY | |
| and a 682-RRAR- | NENGTITDAVDCALDPLSETKCTLKSFTVEKGIYQTSNFRVQPTESIV | |
| 685 - 682-QQAQ- | RFPNITNLCPFGEVFNATRFASVYAWNRKRISNCVADYSVLYNSAS | |
| 685 mutation | FSTFKCYGVSPTKLNDLCFTNVYADSFVIRGDEVRQIAPGQTGKIAD | |
| | YNYKLPDDFTGCVIAWNSNNLDSKVGGNYNYLYRLFRKSNLKPFER | |
| | DISTEIYQAGSTPCNGVEGFNCYFPLQSYGFQPTNGVGYQPYRVV | |
| | LSFELLHAPATVCGPKKSTNLVKNKCVNFNFNGLTGTGVLTESNKK | |
| | FLPFQQFGRDIADTTDAVTRAGCLIGAEHVNNSYECDIPIGAGICASY | |
| | QTQTNRDPQTLEILDITPCSFGGVSVITPGTNTSNQVAVLYQDVNCT | |
| | EVPVAIHADQLTPTWRVYSTGSNVFQSPQQAQSVASQSIIAYTMSL | |
| | GAENSVAYSNNSIAIPTNFTISVTTEILPVSMTKTSVDCTMYICGDST | |
| | ECSNLLLQYGSFCTQLNRALTGIAVEQDKNTQEVFAQVKQIYKTPPI | |
| | KDFGGFNFSQILPDPSKPSKRSPIEDLLFNKVTLADAGFIKQYGDCL | |
| | GDIAARDLICAQKFNGLTVLPPLLTDEMIAQYTSALLAGTITSGWTFG | |
| | AGPALQIPFPMQMAYRFNGIGVTQNVLYENQKLIANQFNSAIGKIQD | |
| | SLSSTPSALGKLQDVVNQNAQALNTLVKQLSSNFGAISSVLNDILSR | |
| | LDPPEAEVQIDRLITGRLQSLQTYVTQQLIRAAEIRASANLAATKMSE | |
| | CVLGQSKRVDFCGKGYHLMSFPQSAPHGVVFLHVTYVPAQEKNFT | |
| | TAPAICHDGKAHFPREGVFVSNGTHWFVTQRNFYEPQIITTDNTFVS | |
| | GNCDWIGIVNNTVYDPLQPELDSFKEELDKYFKNHTSPDVDLGDIS | |
| | GINASWNIQKEIDRLNEVAKNLNESLIDLQELGKYEQYIKWPWYIWL | |
| | GFIAGLIAIVMVTIMLCCMTSCCSCLKGCCSCGSCCKFDEDDSEPVL | |
| | KGVKLHYT | |
| Wild type native | MFVFLVLLPLVSS | 63 |
| leader sequence | | |

Depending on the specific mutation and wherein the genome the nucleotide substitution, addition or deletion occurs, mutations may be neutral, beneficial, or harmful to an organism. Spike (S) protein of SARS-CoV-2 is 1273 amino acids long and is the main target of current COVID-19 vaccines, as well as those in development. It is the portion of the virus that recognizes and binds to host cellular receptors and mediates viral entry. SARS-CoV-2 is unable to infect host cells without it. Because of this, mutations in the S gene, particularly those that affect portions of the protein that are critical for pathogenesis and normal function (such as the receptor-binding domain (RBD) or furin cleavage site) or those that cause conformational changes to the S protein, are of the most significant interest. If “first-wave” antibodies do not recognize these changes, these mutations may provide an avenue for the virus to escape from immunity to the original SARS-CoV-2 strain. In the current study, using robust immunoinformatic approaches, potential human B cell, CD8⁺, and CD4⁺ T cell-specific epitopes were identified in context to a spike glycoprotein region substituted with all the known 36 non-synonymous mutations that constitute the 17 SARS-CoV-2 specific Variants of Concern (VOC). Usage of these epitopes in a multi-epitope vaccine candidate will aid in combating a higher degree of hospitalization and deaths caused due to the highly severe SARS-CoV-2 VOCs and bring a curb on the pandemic.

In some embodiments, the one or more large sequences are derived from a whole protein sequence expressed by SARS-CoV-2. In some embodiments, the one or more large sequences are derived from a partial protein sequence expressed by SARS-CoV-2. In other embodiments, the one or more large conserved sequences is derived from a full-length spike glycoprotein. In some embodiments, the one or more large conserved sequences is derived from a partial spike glycoprotein. In some embodiments the spike (S) protein comprises at least one proline substitution, or at least two proline substitution, or at least, four proline substitution, or at least six proline substitution. The spike (S) protein may comprise two consecutive proline substitutions at amino acid positions 986 and 987. The proline substitutions may comprise K986P and V987P mutations. In further embodiments, the spike (S) protein is receptor-binding domain (RBD). In some embodiments, the RBD comprises a trimerized SARS-CoV-2 receptor-binding domain (RBD).

As previously discussed, each of the large sequences are separated by a linker. In some embodiments, the linker is the same linker. In some embodiments, one or more linkers are different. For example, in some embodiments, a different linker is used between each large sequence. As previously discussed, non-limiting examples of linkers include T2A, E2A, P2A, or the like.

As previously discussed, in certain embodiments, the vaccine delivery system comprises an adenovirus such as but not limited to Ad5. Ad26, Ad35, etc., as well as carriers such as lipid nanoparticles, polymers, peptides, etc.

### CD8+ Epitopes

Examples of methods for identifying potential CD8+ T cell epitopes and screening conservancy of potential CD8+ T cell epitopes are described herein. The present invention is not limited to the particular software systems disclosed, and other software systems are accessible to one of ordinary skill in the art for such methods. The present invention is not limited to the specific haplotypes used herein. For example, one of ordinary skill in the art may select alternative molecules (e.g., HLA molecules) for molecular docking studies,

FIG. 10 shows sequence homology analysis for screening conservancy of potential CD8+ T cell epitopes, e.g., the comparison of sequence homology for the potential CD8+ T cell epitopes among 81,963 SARS-CoV-2 strains (that currently circulate in 190 countries on 6 continents), the 4 major “common cold” Coronaviruses that cased previous outbreaks (e.g. hCoV-OC43, hCoV-229E, hCoV-HKU1-Genotype B, and hCoV-NL63), and the SL-CoVs that were isolated from bats, civet cats, pangolins and camels. Epitope sequences highlighted in yellow present a high degree of homology among the currently circulating 81,963 SARS-CoV-2 strains and at least a 50% conservancy among two or more humans SARS-CoV strains from previous outbreaks, and the SL-CoV strains isolated from bats, civet cats, pangolins and camels.

From the analysis, 27 CD8+ T cell epitopes were selected as being highly conserved. FIG. 11A and FIG. 11B show the docking of the conserved epitopes to the groove of HLA-A*02:01 molecules as well as the interaction scores determined by protein-peptide molecular docking analysis.

FIG. 12A, FIG. 12B, and FIG. 12C shows that CD8+ T cells specific to several highly conserved SARS-CoV-2 epitopes disclosed herein were detected in COVID-19 patients and unexposed healthy individuals. FIG. 13A, FIG. 13B, FIG. 13C, and FIG. 13D shows immunogenicity of the identified SARS-CoV-2 CD8+ T cell epitopes.

The CD8⁺ T cell target epitopes discussed above include S₂₋₁₀, S₁₂₂₀₋₁₂₂₈, S₁₀₀₀₋₁₀₀₈, S₉₅₈₋₉₆₆, E₂₀₋₂₈, ORF1ab₁₆₇₅₋₁₆₈₃, ORF1ab₂₃₆₃₋₂₃₇₁, ORF1ab₃₀₁₃₋₃₀₂₁, ORF1ab₃₁₃₃₋₃₁₉₁, ORF1ab₅₄₇₀₋₅₄₇₈, ORF1ab₆₇₄₉₋₆₇₅₇, ORF7b₂₆₋₃₄, ORF8a₇₃₋₈₁, ORF10₃₋₁₁, and ORF10₅₋₁₃. FIG. 14 shows the genome-wide location of the epitopes. Thus, in certain embodiments, the vaccine composition may comprise one or more CD8⁺ T cell epitopes selected from: S₂₋₁₀, S₁₂₂₀₋₁₂₂₈, S₁₀₀₀₋₁₀₀₈, S₉₅₈₋₉₈₆, E₂₀₋₂₈, ORF1ab₁₆₇₅₋₁₆₈₃, ORF1ab₂₃₆₃₋₂₃₇₁, ORF1ab₃₀₁₃₋₃₀₂₁, ORF1ab₃₁₈₃₋₃₁₉₁, ORF1ab₅₄₇₀₋₅₄₇₈, ORF1ab₆₇₄₉₋₆₇₅₇, ORF7b₂₆₋₃₄, ORF8a₇₃₋₈₁, ORF10₃₋₁₁, ORF10₅₋₁₃, or a combination thereof. Table 3 below describes the sequences for the aforementioned epitope regions.

TABLE 3

| | | SEQ |
|---|---|---|
| CD8⁺ T Cell | Epitope | ID |
| Epitope | Sequence | NO: |
| ORF1ab₈₄₋₉₂ | VMVELVAEL | 2 |
| ORF1ab₁₆₇₅₋₁₅₈₃ | YLATALLTL | 3 |
| ORF1ab₂₂₁₀₋₂₂₁₈ | CLEASFNYL | 4 |
| ORF1ab₂₃₈₃₋₂₃₇₁ | WLMWLIINL | 5 |
| ORF1ab₃₀₁₃₋₃₀₂₁ | SLPGVFCGV | 6 |
| ORF1ab₃₁₈₃₋₃₁₉₁ | FLLNKEMYL | 7 |
| ORF1ab₃₇₃₂₋₃₇₄₀ | SMWALIISV | 8 |
| ORF1ab₄₂₈₃₋₄₂₉₁ | YLASGGQPI | 9 |
| ORF1ab₅₄₇₀₋₅₄₇₈ | KLSYGIATV | 10 |
| ORF1ab₆₄₁₉₋₆₄₂₇ | YLDAYNMMI | 11 |
| ORF1ab_{6749-B757} | LLLDDFVEI | 12 |
| S₂₋₁₀ | FVFLVLLPL | 13 |
| S₆₉₁₋₆₉₉ | SIIAYTMSL | 14 |
| S₉₅₈₋₉₅₆ | ALNTLVKQL | 15 |
| S₉₇₆₋₉₈₄ | VLNDILSRL | 16 |
| S₁₀₀₀₋₁₀₀₈ | RLQSLQTYV | 17 |
| S₁₂₂₀₋₁₂₂₈ | FIAGLIAIV | 18 |
| E₂₀₋₂₈ | FLAFVVFLL | 19 |
| E₂₅₋₃₄ | FLLVTLAIL | 20 |
| E₂₆₋₃₄ | FLLNKEMYL | 21 |
| M₅₂₋₆₀ | IFLWLLWPV | 22 |
| M₈₉₋₉₇ | GLMWLSYFI | 23 |
| ORF6₃₋₁₁ | HLVDFQVTI | 24 |
| ORF7b₂₆₋₃₄ | IIFWFSLEL | 25 |
| ORF8a₃₁₋₃₉ | YVVDDPCPI | 26 |
| ORF8a₇₃₋₈₁ | YIDIGNYTV | 27 |
| ORF10₃₋₁₁ | YINVFAFPF | 28 |
| ORF10₅₋₁₃ | NVFAFPFTL | 29 |
| S | KSYGFQPTY | 194 |
| S | VVGNHKYRF | 195 |
| S | YQVGNKPCK | 196 |
| S | CVIAWNSKK | 137 |
| S | KGAKGLNCY | 198 |
| S | SQCVNFTTR | 199 |
| S | NIADYNYKL | 200 |
| S | YLPLKSYGF | 201 |
| S | KCYGVSLNK | 202 |
| S | IYKTPPIKY | 203 |
| S | CVADYSFLY | 204 |
| S | SVYAWDRRK | 205 |
| S | RFFRKSNLK | 206 |
| S | DiSTEIYQV | 207 |
| S | YQPHRVWVL | 208 |
| S | FVIRGDQVK | 209 |
| S | NATKFSSVY | 210 |
| S | NLCPFSEIF | 211 |
| S | KINNCVADY | 213 |

The present invention is not limited to the aforementioned CD8⁺ T cell epitopes. For example, the present invention also includes variants of the aforementioned CD8⁺ T cell epitopes, for example sequences wherein the aforementioned CD8⁺ T cell epitopes are truncated by one amino acid (examples shown below in Table 4).

TABLE 4

| CD8⁺ TCell | Sequence with | |
|---|---|---|
| Epitope | Single AA | SEQ ID |
| Origin: | Truncation | NO: |
| ORF1ab₈₄₋₉₂ | VMVELVAE | 30 |
| ORF1ab₁₆₇₅₋₁₅₈₃ | LATALLTL | 31 |
| ORFIab₂₂₁₀₋₂₂₁₈ | CLEASFNY | 32 |
| ORFIab₂₃₈₃₋₂₃₇₁ | LMWLIINL | 33 |
| ORFIab₃₀₁₃₋₃₀₂₄ | SLPGVFCG | 34 |
| ORFIab₃₁₈₃₋₃₁₉₁ | LLNKEMYL | 35 |
| ORF1ab₃₇₃₂₋₃₇₄₀ | SMWALIIS | 36 |
| ORF1ab₄₂₈₃₋₄₂₉₁ | LASGGQPI | 37 |
| ORF1ab₅₄₇₀₋₅₄₇₈ | KLSYGIAT | 38 |
| ORF1ab₆₄₁₉₋₅₄₂₇ | LDAYNMMI | 39 |
| ORF1ab₆₇₄₉₋₈₇₅₇ | LLLDDFVE | 40 |
| S₂₋₁₀ | VFLVLLPL | 41 |
| S₆₉₁₋₆₉₉ | SIIAYTMS | 42 |
| S₉₅₈₋₉₆₆ | LNTLVKQL | 43 |
| S₉₇₆₋₉₈₄ | VLNDILSR | 44 |
| S₁₀₀₀₋₁₀₀₈ | LQSLQTYV | 45 |
| S₁₂₂₀₋₁₂₂₈ | FIAGLIAI | 46 |
| E₂₀₋₂₈ | LAFVVFLL | 47 |
| E₂₈₋₃₄ | FLLVTLAL | 48 |
| E₂₈₋₃₄ | LLNKEMYL | 49 |
| M₅₂₋₆₀ | IFLWLLWP | 59 |
| M₈₉₋₉₇ | LMWLSYFI | 51 |
| ORF₆₃₋₁₁ | HLVDFQVT | 52 |
| ORF7b₂₈₋₃₄ | IFWFSLEL | 53 |
| ORF8a₃₁₋₃₉ | YVVDDPCP | 54 |
| ORF8a₇₃₋₈₁ | iDIGNYTV | 55 |
| ORF10₃₋₁₁ | YINVFAFP | 56 |
| ORF10₅₋₁₃ | VFAFPFTI | 57 |
| S | KSYGFQPT | 214 |
| S | VVGNHKYR | 215 |
| S | YQVGNKPC | 216 |
| S | CVIAWNSK | 217 |
| S | KGAKGLNC | 218 |
| S | SQCVNFTT | 219 |
| S | NIADYNYK | 220 |
| S | YLPLKSYG | 221 |
| S | KCYGVSLN | 222 |
| S | IYKTPPIK | 223 |
| S | CVADYSFL | 224 |
| S | SVYAWDRR | 225 |
| S | RFFRKSNL | 226 |
| S | DiSTEIYQ | 227 |
| S | YQPHRVVV | 228 |
| S | FVIRGDQV | 229 |
| S | NATKFSSV | 230 |
| S | NLCPFSEI | 231 |
| S | ASATVCGP | 232 |
| S | KINNCVAD | 233 |
| S | KSYGFQPT | 234 |

The present invention is not limited to the aforementioned CD8⁺ T cell epitopes.

In certain embodiments, the vaccine composition comprises 1-10 CD8⁺ T cell target epitopes. In certain embodiments, the vaccine composition comprises 2-10 CD8⁺ T cell target epitopes. In certain embodiments, the vaccine composition comprises 2-15 CD8⁺ T cell target epitopes. In certain embodiments, the vaccine composition comprises 2-20 CD8⁺ T cell target epitopes. In certain embodiments, the vaccine composition comprises 2-30 CD8⁺ T cell target epitopes. In certain embodiments, the vaccine composition comprises 2-15 CD8⁺ T cell target epitopes. In certain embodiments, the vaccine composition comprises 2-5 CD8⁺ T cell target epitopes. In certain embodiments, the vaccine composition comprises 5-10 CD8⁺ T cell target epitopes. In certain embodiments, the vaccine composition comprises 5-15 CD8⁺ T cell target epitopes. In certain embodiments, the vaccine composition comprises 5-20 CD8⁺ T cell target epitopes. In certain embodiments, the vaccine composition comprises 5-25 CD8⁺ T cell target epitopes. In certain embodiments, the vaccine composition comprises 5-30 CD8⁺ T cell target epitopes. In certain embodiments, the vaccine composition comprises 10-20 CD8⁺ T cell target epitopes. In certain embodiments, the vaccine composition comprises 10-30 CD8⁺ T cell target epitopes

### CD4+ Epitopes

Examples of methods for identifying potential CD4+ T cell epitopes and screening conservancy of potential CD4+ T cell epitopes are described herein. The present invention is not limited to the particular software systems disclosed, and other software systems are accessible to one of ordinary skill in the art for such methods. The present invention is not limited to the specific haplotypes used herein. For example, one of ordinary skill in the art may select alternative molecules (e.g., HLA molecules) for molecular docking studies,

FIG. 15 shows the identification of highly conserved potential SARS-CoV-2-derived human CD4+ T cell epitopes that bind with high affinity to HLA-DR molecules. Out of a total of 9,594 potential HLA-DR-restricted CD4+ T cell epitopes from the whole genome sequence of SARS-CoV-2-Wuhan-Hu-1 strain (MN908947.3), 16 epitopes that bind with high affinity to HLA-DRB1 molecules were selected. The conservancy of the 16 CD4+ T cell epitopes was analyzed among human and animal Coronaviruses Shown are the comparison of sequence homology for the 16 CD4+ T cell epitopes among 81,963 SARS-CoV-2 strains (that currently circulate in 6 continents), the 4 major “common cold” Coronaviruses that cased previous outbreaks (i.e. hCoV-OC43, hCoV-229E, hCoV-HKU1, and hCoV-NL63), and the SL-CoVs that were isolated from bats, civet cats, pangolins and camels. Epitope sequences highlighted in green present high degree of homology among the currently circulating 81,963 SARS-CoV-2 strains and at least a 50% conservancy among two or more humans SARS-CoV strains from previous outbreaks, and the SL-CoV strains isolated from bats, civet cats, pangolins and camels.

From the analysis, 16 CD4+ T cell epitopes were selected as being highly conserved. FIG. 16A and FIG. 16B show the docking of the conserved epitopes to the groove of HLA-A*02:01 molecules as well as the interaction scores determined by protein-peptide molecular docking analysis.

FIG. 17A, FIG. 17B, and FIG. 17C show that CD4+ T cells specific to several highly conserved SARS-CoV-2 epitopes disclosed herein were detected in COVID-19 patients and unexposed healthy individuals. FIG. 18A, FIG. 18B, FIG. 18C, and FIG. 18D show immunogenicity of the identified SARS-CoV-2 CD4+ T cell epitopes.

The CD4⁺ T cell target epitopes discussed above include ORF1a₁₃₅₀₋₁₃₆₅, ORF1ab₅₀₁₉₋₅₀₃₃, ORF6₁₂₋₂₆, ORF1ab₆₀₈₆₋₆₁₀₂, ORF1ab₆₄₂₀₋₆₄₃₄, ORF1a₁₈₀₁₋₁₈₁₅, S₁₋₁₃, E₂₆₋₄₀, E₂₀₋₃₄, M₁₇₆₋₁₉₀, N₂₈₈₋₄₀₃, ORF7a₃₋₁₇, ORF7a₁₋₁₅, ORF7b₈₋₂₂, ORF7a₉₆₋₁₁₂, and ORF8₁₋₁₅. FIG. 14 shows the genome-wide location of the epitopes. Thus, in certain embodiments, the vaccine composition may comprise one or more CD4⁺ T cell target epitopes selected from ORF1a₁₃₅₀₋₁₃₆₅, ORF1ab₅₀₁₉₋₅₀₃₃, ORF6₁₂₋₂₆, ORF1ab₆₀₈₆₋₆₁₀₂, ORF1ab₆₄₂₀₋₆₄₃₄, ORF1a₁₈₀₁₋₁₈₁₅, S₁₋₁₃, E₂₆₋₄₀, E₂₀₋₃₄, M₁₇₆₋₁₉₀, N₃₈₈₋₄₀₃, ORF7a₃₋₁₇, ORF7a₁₋₁₅, ORF7b₈₋₂₂, ORF7a₉₈₋₁₁₂, ORF8₁₋₁₅, or a combination thereof. Table 5 below describes the sequences for the aforementioned epitope regions.

TABLE 5

| CD4⁺ | | SEQ |
|---|---|---|
| T Cell | | ID |
| Epitope | Epitope Sequence | NO: |
| ORF1a₁₃₅₀₋₁₃₈₅ | KSAFYILPSIISNEK | 58 |
| ORF1a₁₈₀₁₋₁₈₁₅ | ESPFVMMSAPPAQYE | 59 |
| ORF1ab₅₀₁₉₋₅₀₃₃ | PNMLRIMASLVLARK | 60 |
| ORF1ab₆₀₈₈₋₆₁₀₂ | RIKVQMLSDTLKNL | 61 |
| ORF1ab₆₄₂₀₋₆₄₃₄ | LDAYNMMISAGFSLW | 62 |
| S₁₋₁₃ | MFVFLVLLPLVSS | 63 |
| E₂₀₋₃₄ | FLAFVVFLLVTLAIL | 64 |
| E₂₈₋₄₀ | FLLVTLAILTALRLC | 65 |
| M₁₇₅₋₁₉₀ | LSYYKLGASQRVAGD | 66 |
| ORF6ₗ₂₋₂₆ | AEILLIIMRTFKVSI | 67 |
| ORF7a₁₋₁₅ | MKIILFLALITLATC | 68 |
| ORF7a₃₋₁₇ | IIFLALITLATCEL | 69 |
| ORF7a₉₆₋₁₁₂ | SPIFLIVAAIVFITL | 70 |
| ORF7b₈₋₂₂ | DFYLCFLAFLLFLVL | 71 |
| ORFBb₁₋₁₅ | MKFLVFLGIITTVAA | 72 |
| N₃₈₈₋₄₀₃₁ | KQQTVTLLPAADLDDF | 73 |
| S | LCPFSEIFNATKFSS | 235 |
| S | NCYLPLKSYGFQPTY | 236 |
| S | GNHKYRFRFFRKSNL | 237 |
| S | PFERDISTEIYQVGN | 238 |
| S | KKLDSKVVGNHKYRF | 239 |
| S | KGLNCYLPLKSYGFQ | 240 |
| S | LVLLPLVSSQCVNFT | 241 |
| S | RGDQVKQIAPGQTGN | 242 |
| S | SASFSTFKCYGVSLN | 243 |
| S | KLDSKVVGNHKYRFR | 244 |
| S | FAQVKQIYKTPPIKY | 245 |
| S | ADYSFLYNSASFSTF | 246 |
| S | ATKFSSVYAWDRRKI | 247 |
| S | PHRVVVLSFELLHAS | 248 |
| S | FERDISTEIYQVGNK | 249 |
| S | AKGLNCYLPLKSYGF | 250 |
| S | SIVRFPNITNLCPFS | 251 |
| S | NNCVADYSFLYNSAS | 252 |
| S | KGAKGLNCYLPLKSY | 253 |

The present invention is not limited to the aforementioned CD4⁺ T cell epitopes. For example, the present invention also includes variants of the aforementioned CD4⁺ T cell epitopes, for example sequences wherein the aforementioned CD42 T cell epitopes are truncated by one or more amino acids or extended by one or more amino acids (examples shown below in Table 6).

TABLE 6

| CD4⁺ T Cell | Sequence | SEQ |
|---|---|---|
| Epitope | with Single | ID |
| Origin | AA Truncation | NO: |
| ORF1a₁₃₅₀₋₁₃₈₅ | KSAFYILPSIISNE | 74 |
| ORF1a₁₈₀₁₋₁₈₁₅ | ESPFVMMSAPPAQY | 75 |
| ORF1ab₅₀₁₉₋₅₀₃₃ | PNMLRIMASLVLAR | 76 |
| ORF1ab₆₀₈₈₋₈₁₀₂ | RIKVQMLSDTLKN | 77 |
| ORF1ab₆₄₂₀₋₈₄₃₄ | LDAYNMMISAGFSL | 78 |
| S₁₋₁₃ | MFVFLVLLPLVS | 79 |
| E₂₀₋₃₄ | FLAFVVFLLVTLAL | 80 |
| E₂₈₋₄₀ | FLLVTLAILTALRL | 81 |
| M₁₇₅₋₁₉₀ | LSYYKLGASQRVAG | 82 |
| ORF6₁₂₋₂₆ | AEILLIIMRTFKVS | 83 |
| ORF7a₁₋₁₅ | MKIILFLALITLAT | 84 |
| ORF7a₃₋₁₇ | IFLALITLATCE | 85 |
| ORF7a₉₈₋₁₁₂ | SPIFLIVAAIVFIT | 86 |
| ORF7b₈-₂₂ | DFYLCFLAFLLFLV | 87 |
| ORF8b₁₋₁₅ | MKFLVFLGIITTVA | 88 |
| N₃₈₈₋₄₀₃₁ | KQQTVTLLPAADLDD | 89 |
| ORF1a₁₂₅₀₋₁₃₈₅ | SAFYILPSIISNEK | 90 |
| ORF1a₁₈₀₁₋₁₈₁₅ | SPFVMMSAPPAQYE | 91 |
| ORF1ab₅₀₁₉₋₅₀₃₃ | NMLRIMASLVLARK | 92 |
| ORF1ab₆₀₈₈₋₆₁₀₂ | IKVQMLSDTLKNL | 93 |
| ORF1ab₆₄₂₀₋₆₄₃₄ | DAYNMMISAGFSLW | 94 |
| S₁₋₁₃ | FVFLVLLPLVSS | 95 |
| E₂₀₋₃₄ | LAFVVFLLVTLAIL | 96 |
| E₂₈₋₄₀ | LLVTLAILTALRLC | 97 |
| M₁₇₆₋₁₉₀ | SYYKLGASQRVAGD | 98 |
| ORF6₁₂₋₂₆ | EILLI IMRTFKVSI | 99 |
| ORF7a₁₋₁₅ | KIILFLALITLATC | 100 |
| ORF7a₃₋₁₇ | IFLALITLATCEL | 101 |
| ORF7a₉₈₋₁₁₂ | PIFLIVAAIVFITL | 102 |
| ORF7b₈₋₂₂ | FYLCFLAFLLFLVL | 103 |
| ORF8b1-15 | KFLVFLGIITTVAA | 104 |
| N₃₈₈₋₄₀₃₁ | QQTVTLLPAADLDDF | 105 |
| S | LCPFSEIFNATKFS | 254 |
| S | NCYLPLKSYGFQPT | 255 |
| S | GNHKYRFRFFRKSN | 256 |
| S | PFERDISTEIYQVG | 257 |
| S | KKLDSKVVGNHKYR | 258 |
| S | KGLNCYLPLKSYGF | 259 |
| S | LVLLPLVSSQCVNF | 260 |
| S | RGDQVKQIAPGQTG | 261 |
| S | SASFSTFKCYGVSL | 262 |
| S | KLDSKWGNHKYRF | 263 |
| S | FAQVKQIYKTPPIK | 264 |
| S | ADYSFLYNSASFST | 265 |
| S | ATKFSSVYAWDRRK | 266 |
| S | PHRVVVLSFELLHA | 267 |
| S | FERDISTEIYQVGN | 268 |
| S | AKGLNCYLPLKSYG | 269 |
| S | SIVRFPNITNLCPF | 270 |
| S | NNCVADYSFLYNSA | 271 |
| S | KGAKGLNCYLPLKS | 272 |

The present invention is not limited to the aforementioned CD4⁺ T cell epitopes.

In certain embodiments, the vaccine composition comprises 1-10 CD4⁺ T cell target epitopes. In certain embodiments, the vaccine composition comprises 2-10 CD4⁺ T cell target epitopes. In certain embodiments, the vaccine composition comprises 2-15 CD4⁺ T cell target epitopes. In certain embodiments, the vaccine composition comprises 2-20 CD4⁺ T cell target epitopes. In certain embodiments, the vaccine composition comprises 2-30 CD4⁺ T cell target epitopes. In certain embodiments, the vaccine composition comprises 2-15 CD4⁺ T cell target epitopes. In certain embodiments, the vaccine composition comprises 2-5 CD4⁺ T cell target epitopes. In certain embodiments, the vaccine; composition comprises 5-10 CD4⁺ T cell target epitopes. In certain embodiments, the vaccine composition comprises 5-15 CD4+ T cell target epitopes. In certain embodiments, the vaccine composition comprises 5-20 CD4⁺ T cell target epitopes. In certain embodiments, the vaccine composition comprises 5-25 CD4⁺ T cell target epitopes. In certain embodiments, the vaccine composition comprises 5-30 CD4⁺ T cell target epitopes. In certain embodiments, the vaccine composition comprises 10-20 CD4⁺ T cell target epitopes. In certain embodiments, the vaccine composition comprises 10-30 CD4⁺ T cell target epitopes.

### B Cell Epitopes

Examples of methods for identifying potential B cell epitopes and screening conservancy of potential B cell epitopes are described herein. The present invention is not limited to the particular software systems disclosed, and other software systems are accessible to one of ordinary skill in the art for such methods.

FIG. 19 shows the conservation of Spike-derived B cell epitopes among human, bat, civet cat, pangolin, and camel coronavirus strains. Multiple sequence alignment performed using ClustalW among 29 strains of SARS coronavirus (SARS-CoV) obtained from human, bat, civet, pangolin, and camel. This includes 7 human SARS/MERS-CoV strains (SARS-CoV-2-Wuhan (MN908947.3), SARS-HCoV-Urbani (AY278741.1), CoV-HKU1-Genotype-B (AY884001), CoV-OC43 (KF923903), CoV-NL63 (NC005831), CoV-229E (KY983587), MERS (NC019843)); 8 bat SARS-CoV strains (BAT-SL-CoV-WIV16 (KT444582), BAT-SL-CoV-WIV1 (KF367457.1), BAT-SL-CoV-YNLF31C (KP886808.1), BAT-SARS-CoV-RS672 (FJ588686.1), BAT-CoV-RATG13 (MN996532.1), BAT-CoV-YN01 (EPIISL412976), BAT-CoV-YNO2 (EPIISL412977), BAT-CoV-19-ZXC21 (MG772934.1); 3 Civet SARS-CoV strains (SARS-CoV-Civet007 (AY572034.1), SARS-CoV-A022 (AY686863.1), SARS-CoV-B039 (AY686864.1)); 9 pangolin SARS-CoV strains (PCoV-GX-P2V (MT072864.1), PCoV-GX-P5E (MT040336.1), PCoV-GX-P5L (MT040335.1), PCoV-GX-P1E (MT040334.1), PCoV-GX-P4L (MT040333.1), PCoV-MP789 (MT084071.1), PCoV-GX-P3B (MT072865.1), PCoV-Guangdong-P2S (EPIISL410544), PCoV-Guangdong (EPIISL410721)); 4 camel SARS-CoV strains (Camel-CoV-HKU23 (KT368891.1), DcCoV-HKU23 (MN514967.1), MERS-CoV-Jeddah (KF917527.1), Riyadh/RY141 (NC028752.1)) and 1 recombinant strain (FJ211859.1)). Regions highlighted with blue color represent the sequence homology. The B cell epitopes, which showed at least 50% conservancy among two or more strains of the SARS Coronavirus or possess receptor-binding domain (RBD) specific amino acids were selected as candidate epitopes.

From the analysis, 22 B cell epitopes were selected as being highly conserved. FIG. 20A and FIG. 20B shows the docking of the conserved epitopes to the ACE2 receptor as well as the interaction scores determined by protein-peptide molecular docking analysis. FIG. 21A, FIG. 21B, FIG. 21C, FIG. 21D, FIG. 21E, FIG. 21F, and FIG. 21G shows immunogenicity of the identified SARS-CoV-2 B cell epitopes.

The B cell target epitopes discussed above include S₂₈₇₋₃₁₇, S₅₂₄₋₅₉₈, S₆₀₁₋₆₄₀, S₈₀₂₋₈₁₉, S₈₈₈₋₉₀₉, S₃₆₉₋₃₉₃, S₄₄₀₋₅₀₁, S₁₁₃₃₋₁₁₇₂, S₃₂₉₋₃₆₃, S₅₉₋₈₁, and S₁₃₋₃₇. FIG. 28 shows the genome-wide location of the epitopes. Thus, in certain embodiments, the vaccine composition may comprise one or more B cell target epitopes selected from: S₂₃₇₋₃₁₇, S₅₂₄₋₅₉₈, S₆₀₁₋₆₄₀, S₈₀₂₋₈₁₉, S₈₈₈₋₉₀₉, S₃₅₉₋₃₉₃, S₄₄₀₋₅₀₁, S₁₁₃₃₋₁₁₇₂, S₃₂₉₋₃₆₃, S₅₉₋₈₁, and S₁₃₋₃₇. In some embodiments; the B cell epitope is whole spike protein. In some embodiments, the B cell epitope is a portion of the spike protein. Table 7 below describes the sequences for the aforementioned epitope regions.

TABLE 7

| | | SEQ |
|---|---|---|
| B Cell | | ID |
| Epitope | Epitope Sequence | NO: |
| S₁₃₋₃₇ | SQCVNLTTRTQLPPAYTNSFT | 106 |
| | RGVY | |
| S₅₉₋₈₁ | FSNVTWFHAIHVSGTNGTKRF | 107 |
| | DN | |
| S₂₈₇₋₃₁₇ | DAVDCALDPLSETKCTLKSFT | 108 |
| | VEKGIYQTSN | |
| S₆₀₁₋₆₄₀ | GTNTSNQVAVLYODVNCTEV | 109 |
| | PVAIHADQLTPTWRVYSTGS | |
| S₅₂₄₋₅₉₈ | VCGPKKSTNLVKNKCVNFNFN | 110 |
| | GLTGTGVLTESNKKFLPFQQF | |
| | GRDIADTTDAVRDPQTLEILDI | |
| | TPCSFGGVSVI | |
| S₄₄₀₋₅₀₁ | NLDSKVGGNYNYLYRLFRKSN | 111 |
| | LKPFERDISTEIYQAGSTPCNG | |
| | VEGFNCYFPLQSYGFQPTE | |
| S₃₆₉₋₃₉₃ | YNSASFSTFKCYGVSPTKLND | 112 |
| | LCFT | |
| S₃₂₉₋₃₆₃ | FPNITNLCPFGEVFNATRFASV | 113 |
| | YAWNRKRISNCVA | |
| S₁₁₃₃₋₁₁₇₂ | VNNTVYDPLQPELDSFKEELD | 114 |
| | KYFKNHTSPDVDLGDISGI | |
| S₈₀₂₋₈₁₉ | FSQILPDPSKPSKRSFIE | 115 |
| S₈₈₈₋₉₀₉ | FGAGAALQIPFAMQMAYRFN | 116 |
| | GI | |
| S | CVNFTTRTQLPPAYTNSFT | 273 |
| | RGVYY | |
| S | NITNLCPFSEIFNATKFSSV | 274 |
| | YAWDRR | |
| S | INNCVADYSFLYNSASFST | |
| | FKCYGVSLNKLNDL | |
| S | RGDQVKQIAPGQTGNIAD | 276 |
| S | KKLDSKWGNHKYRFRFFR | 277 |
| | KSNLKPFERDISTEISTEIY | |
| | QVGNKPCKG | |
| S | TYGVGY | 278 |
| S | LHASATVCGPKKSTNL | 279 |
| S | VKQIYKTPPIKYFGGFNFS | 280 |
| | QILPDPSKPSK | |

The present invention is not limited to the aforementioned B cell epitopes. For example, the present invention also includes variants of the aforementioned B cell epitopes, for example sequences wherein the aforementioned B cell epitopes are truncated by one or more amino acids or extended by one or more amino acids (examples shown below in Table 8).

TABLE 8

| Origin | | SEQ |
|---|---|---|
| of | Sequence with AA | ID |
| Epitope | Truncation | NO: |
| S₁₃₋₃₇ | SQCVNLTTRTQLPPAYTNSFT | 117 |
| | RG | |
| S₅₉₋₇₉ | FSNVTWFHAIHVSGTNGTKRF | 118 |
| S₂₈₇₋₃₁₅ | DAVDCALDPLSETKCTLKSFT | 119 |
| | VEKGIYQT | |
| S₆₀₁₋₅₃₈ | GTNTSNQVAVLYQDVNCTEV | 120 |
| | PVAIHADQLTPTWRVYST | |
| S₅₂₄₋₅₉₆ | VCGPKKSTNLVKNKCVNFNFN | 121 |
| | GLTGTGVLTESNKKFLPFQQF | |
| | GRDIADTTDAVRDPQTLEILDI | |
| | TPCSFGGVS | |
| S₄₄₀₋₄₉₉ | NLDSKVGGNYNYLYRLFRKSN | 122 |
| | LKPFERDISTEIYQAGSTPCNG | |
| | VEGFNCYFPLQSYGFQP | |
| S₃₆₉₋₃₉₁ | YNSASFSTFKCYGVSPTKLND | 123 |
| | LC | |
| S₃₂₉₋₃₆₁ | FPNITNLCPFGEVFNATRFASV | 124 |
| | YAWNRKRISNC | |
| S₁₁₃₃₋₁₁₇₀ | VNNTVYDPLQPELDSFKEELD | 125 |
| | KYFKNHTSPDVDLGDIS | |
| S₈₀₂₋₈₁₇ | FSQILPDPSKPSKRSF | 126 |
| S₈₈₈₋₉₀₇ | FGAGAALQIPFAMOMAYRFN | 127 |
| S₁₃₋₃₇ | CVNLTTRTQLPPAYTNSFT | 128 |
| | RGVY | |
| S₆₁₋₈₁ | NVTWFHAIHVSGTNGTKR | 129 |
| | FDN | |
| S₂₈₇₋₃₁₇ | VDCALDPLSETKCTLKSFT | 130 |
| | VEKGIYQTSN | |
| S₆₀₁₋₆₄₀ | NTSNQVAVLYQDVNCTEV | 131 |
| | PVAIHADQLTPTWRVYSTG | |
| | S | |
| S₅₂₄₋₅₉₈ | GPKKSTNLVKNKCVNFNF | 132 |
| | NGLTGTGVLTESNKKFLPF | |
| | QQFGRDIADTTDAVRDPQ | |
| | TLEILDITPCSFGGVSVI | |
| S₄₄₀₋₅₀₁ | DSKVGGNYNYLYRLFRKS | 133 |
| | NLKPFERDISTEIYQAGSTP | |
| | CNGVEGFNCYFPLQSYGF | |
| | QPTE | |
| S₃₇₁₋₃₉₃ | SASFSTFKCYGVSPTKLND | 134 |
| | LCFT | |
| S₃₃₁₋₃₈₃ | NITNLCPFGEVFNATRFAS | 135 |
| | VYAWNRKRISNCVA | |
| S₁₁₃₅₋₁₁₇₂ | NTVYDPLQPELDSFKEELD | 136 |
| | KYFKNHTSPDVDLGDISGI | |
| S₈₀₄₋₈₁₉ | QILPDPSKPSKRSFIE | 137 |
| S₈₉₀₋₉₀₉ | AGAALQIPFAMQMAYRFN | 138 |
| | GI | |
| S | CVNFTTRTQLPPAYTNSFTRG | 281 |
| | V | |
| S | NITNLCPFSEIFNATKFSSVYA | 282 |
| | WD | |
| S | INNCVADYSFLYNSASFSTFK | 283 |
| | CYGVSLNKLN | |
| S | RGDQVKQIAPGQTGNI | 284 |
| S | KKLDSKWGNHKYRFRFFRKS | 285 |
| | NLKPFERDISTEISTEIYQVGN | |
| | KPC | |
| S | LHASATVCGPKKST | 286 |
| S | VKQIYKTPPIKYFGGFNFSQIL | 287 |
| | PDPSKP | |
| S | NFTTRTQLPPAYTNSFTRG | 288 |
| | VYY | |
| S | TNLCPFSEIFNATKFSSVYA | 289 |
| | WDRR | |
| S | NCVADYSFLYNSASFSTFK | 290 |
| | CYGVSLNKLNDL | |
| S | DQVKQIAPGQTGNIAD | 291 |
| S | LDSKWGNHKYRFRFFRKS | 292 |
| | NLKPFERDISTEISTEIYQV | |
| | GNKPCKG | |
| S | ASATVCGPKKSTNL | 293 |
| S | QIYKTPPIKYFGGFNFSQIL | 294 |
| | PDPSKPSK | |

As previously discussed, in some embodiments, the B cell epitope is in the form of whole spike protein. In some embodiments, the B cell epitope is in the form of a portion of spike protein. In some embodiments, the transmembrane anchor of the spike protein has an intact S1-S2 cleavage site. In some embodiments, the spike protein is in its stabilized conformation. In some embodiments, the spike protein is stabilized with proline substitutions at amino acid positions 986 and 987 at the top of the central helix in the S2 subunit. In some embodiments, the composition comprises a trimerized SARS-CoV-2 receptor-binding domain (RBD). In some embodiments, the trimerized SARS-CoV-2 receptor-binding domain (RBD) sequence is modified by the addition of a T4 fibritin-derived foldon trimerization domain. In some embodiments, the addition of a T4 fibritin-derived foldon trimerization domain increases immunogenicity by multivalent display. FIG. 22 shows a non-limiting example of a spike protein comprising one or more mutations.

In some embodiments, the spike protein comprises Tyr-489 and Asn-487 (e.g., Tyr-489 and Asn-487 help with interaction with Tyr 83 and Gln-24 on ACE-2). In some embodiments, the spike protein comprises Gln-493 (e.g., Gln-493 helps with interaction with Glu-35 and Lys-31 on ACE-2). In some embodiments, the spike protein comprises Tyr-505 (e.g., Tyr-505 helps with interaction with Glu-37 and Arg-393 on ACE-2). In some embodiments, the composition comprises a mutation 682-PRAR-685→682-QQAQ-685 in the S1-S2 cleavage site.

In some embodiments, the composition comprises at least one proline substitution. In some embodiments, the composition comprises at least two proline substitutions. For example, the proline substitution may be at position K986 and V987.

In certain embodiments, the vaccine composition comprises 1-10 B cell target epitopes. In certain embodiments, the vaccine composition comprises 2-10 B cell target epitopes. In certain embodiments, the vaccine composition comprises 2-15 B cell target epitopes. In certain embodiments, the vaccine composition comprises 2-20 B cell target epitopes. In certain embodiments, the vaccine composition comprises 2-30 B cell target epitopes. In certain embodiments, the vaccine composition comprises 2-15 B cell target epitopes. In certain embodiments, the vaccine composition comprises 2-5 B cell target epitopes. In certain embodiments, the vaccine composition comprises 5-10 B cell target epitopes. In certain embodiments, the vaccine composition comprises 5-15 B cell target epitopes. In certain embodiments, the vaccine composition comprises 5-20 B cell target epitopes. In certain embodiments, the vaccine composition comprises 5-25 B cell target epitopes. In certain embodiments, the vaccine composition comprises 5-30 B cell target epitopes. In certain embodiments, the vaccine composition comprises 10-20 B cell target epitopes. In certain embodiments, the vaccine composition comprises 10-30 B cell target epitopes.

For certain embodiments, the epitopes that are selected may be those that achieve a particular score in a binding assay (for binding to an HLA molecule, for example.) For example, in some embodiments, the epitopes selected have an IC₅₀ score of 250 or less in an ELISA binding assay (e.g., an ELISA binding assay specific for HLA-DR/peptide combination, HLA-A*0201/peptide combination, etc.), or the equivalent of the IC₅₀ score of 250 or less in a different binding assay. Binding assays are well known to one of ordinary skill in the art.

### Large Sequence(s) Arrangements

The large sequences of the compositions described may be arranged in various configurations (see FIG. 23). In some embodiments, the large sequences may be arranged such that a spike glycoprotein (S) or a portion thereof (e.g., the RBD) is followed by an ORF1a/b protein or a portion thereof followed by Nucleoprotein or a portion thereof. In some embodiments, the large sequences may be arranged such that a spike glycoprotein (S) or a portion thereof (e.g., the RBD) is followed by an ORF1a/b protein or a portion thereof followed by Nucleoprotein, or a portion thereof is followed by a membrane (M) or a portion thereof.

In some embodiments, the large sequences may be arranged such that an ORF1a/b protein or a portion thereof followed by a nucleoprotein (N) or a portion thereof. In some embodiments, the large sequences may be arranged such that an ORF1a/b protein or a portion thereof followed by nucleoprotein (N), or a portion thereof is followed by a membrane (M) or a portion thereof.

In some embodiments, the large sequences may be arranged such that a spike glycoprotein (S) or a portion thereof (e.g., the RBD) is followed by fragment 1 or a portion thereof. In some embodiments, the large sequences may be arranged such that a spike glycoprotein (S) or a portion thereof (e.g., the RBD) is followed by fragment 2 or a portion thereof. In some embodiments, the large sequences may be arranged such that a spike glycoprotein (S) or a portion thereof (e.g., the RBD) is followed by fragment 4 or a portion thereof. In some embodiments, the large sequences may be arranged such that a spike glycoprotein (S) or a portion thereof (e.g., the RBD) is followed by fragment 5 or a portion thereof. In further embodiments, the large sequences may be arranged such that a spike glycoprotein (S) or a portion thereof (e.g., the RBD) is followed by fragment 1 or a portion thereof, followed by fragment 5 or a portion thereof.

In some embodiments, the large sequences may be arranged such that a spike glycoprotein (S) or a portion thereof (e.g., the RBD) is followed by a nucleocapsid protein or a portion thereof. In some embodiments, the large sequences may be arranged such that a spike glycoprotein (S) or a portion thereof (e.g., the RBD) is followed by a ORF1ab protein or portion thereof, followed by a ORF3 protein or portion thereof followed by an Envelope protein or protein thereof, followed by Membrane protein or portion thereof followed by an ORF6 protein or portion thereof, followed by a ORF7a protein or portion thereof. In some embodiments, the large sequences may be arranged such that a spike glycoprotein (3) or a portion thereof (e.g., the RBD) is followed by a membrane protein or portion thereof, followed by an envelope protein or portion thereof, followed by a Nsp3 protein or portion thereof, followed by a Nsp5 protein or portion thereof, followed by a Nsp12 protein or portion thereof.

In some embodiments, the large sequences may be arranged such that a spike glycoprotein (S) or a portion thereof (e.g., the RBD) is followed by one large sequence. In some embodiments, the large sequences may be arranged such that a spike glycoprotein (3) or a portion thereof (e.g., the RBD) is followed by two large sequences. In some embodiments, the large sequences may be arranged such that a spike glycoprotein (S) or a portion thereof (e.g., the RBD) is followed by three large sequences. In some embodiments, the large sequences may be arranged such that a spike glycoprotein (5) or a portion thereof (e.g., the RBD) is followed by four large sequences. In some embodiments, the large sequences may be arranged such that a spike glycoprotein (S) or a portion thereof (e.g., the RBD) is followed by five large sequences.

In some embodiments, the large sequences may be arranged such that a spike glycoprotein (S) or a portion thereof (e.g., the RBD) is followed by one large sequence both are driven each by a promoter or both are driven by a single promoter but separated by a linker as illustrated in FIG x, y and z)

### Vaccine Candidates

As previously discussed, the present invention provides vaccine compositions comprising an antigen featuring: one or more large sequences, two or more large sequences, three or more large sequences, four or more large sequences, or five or more large sequences. In some embodiment, the large sequences comprise at least one B cell epitope and at least one CD4+ T cell epitope, at least one B cell epitope and at least one CD3+ T cell epitope, at least one CD4+ T cell epitope and at least one CD8+ T cell epitope, or at least one B cell epitope, at least one CD4+ T cell epitope, and at least one CD8+ T cell epitope.

Table 9 and FIG. 24 and FIG. 28 shows examples of vaccine compositions described herein. The present invention is not limited to the examples in Table 9. Per SME TRE with Cl.

TABLE 9

| Vaccine | | SEQ |
|---|---|---|
| Candidate | Sequence: | ID NO: |
| 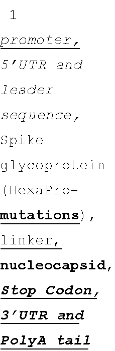 | CTCGACATTGATTATTGACTAGTTATTAATAGTAATCAATTACGGGGTCA TTAGTTCATAGCCCATATATGGAGTTCCGCGTTACATAACTTACGGTAAA TGGCCCGCCTGGCTGACCGCCCAACGACCCCCGCCCATTGACGTCAAT AATGACGTATGTTCCCATAGTAACGCCAATAGGGACTTTCCATTGACGT CAATGGGTGGAGTATTTACGGTAAACTGCCCACTTGGCAGTACATCAAG TGTATCATATGCCAAGTACGCCCCCTATTGACGTCAATGACGGTAAATG GCCCGCCTGGCATTATGCCCAGTACATGACCTTATGGGACTTTCCTACT TGGCAGTACATCTACGTATTAGTCATCGCTATTACCATGGTCGAGGTGA GCCCCACGTTCTGCTTCACTCTCCCCATCTCCCCCCCCTCCCCACCCC CAATTTTGTATTTATTTATTTTTTAATTATTTTGTGCAGCGATGGGGGCG GGGGGGGGGGGGGGGCGCGCGCCAGGCGGGGCGGGGCGGGGCGA GGGGCGGGGCGGGGCGAGGCGGAGAGGTGCGGCGGCAGCCAATCA GAGCGGCGCGCTCCGAAAGTTTCCTTTTATGGCGAGGCGGCGGCGGC GGCGGCCCTATAAAAAGCGAAGCGCGCGGCGGGCGGGAGTCGCTGC GCGCTGCCTTCGCCCCGTGCCCCGCTCCGCCGCCGCCTCGCGCCGCC CGCCCCGGCTCTGACTGACCGCGTTACTCCCACAGGTGAGCGGGCGG GACGGCCCTTCTCCTCCGGGCTGTAATTAGCGCTTGGTTTAATGACGG CTTGTTTCTTTTCTGTGGCTGCGTGAAAGCCTTGAGGGGCTCCGGGAG GGCCCTTTGTGCGGGGGGAGCGGCTCGGGGGGTGCGTGCGTGTGTG TGTGCGTGGGGAGCGCCGCGTGCGGCTCCGCGCTGCCCGGCGGCTG TGAGCGCTGCGGGCGCGGCGCGGGGCTTTGTGCGCTCCGCAGTGTGC GCGAGGGGAGCGCGGCCGGGGGCGGTGCCCCGCGGTGCGGGGGGG | 139 |
| | GCTGCGAGGGGAACAAAGGCTGCGTGCGGGGTGTGTGCGTGGGGGG | |
| | GTGAGCAGGGGGTGTGGGCGCGTCGGTCGGGCTGCAACCCCCCCTG | |
| | CACCCCCCTCCCCGAGTTGCTGAGCACGGCCCGGCTTCGGGTGCGGG | |
| | GCTCCGTACGGGGCGTGGCGCGGGGCTCGCCGTGCCGGGCGGGGGG | |
| | TGGCGGCAGGTGGGGGTGCCGGGCGGGGCGGGGCCGCCTCGGGCC | |
| | GGGGAGGGCTCGGGGGAGGGGCGCGGCGGCCCCCGGAGCGCCGGC | |
| | GGCTGTCGAGGCGCGGCGAGCCGCAGCCATTGCCTTTTATGGTAATCG | |
| | TGCGAGAGGGCGCAGGGACTTCCTTTGTCCCAAATCTGTGCGGAGCCG | |
| | AAATCTGGGAGGCGCCGCCGCACCCCCTCTAGCGGGCGCGGGGCGAA | |
| | GCGGTGCGGCGCCGGCAGGAAGGAAATGGGCGGGGAGGGCCTTCGT | |
| | GCGTCGCCGCGCCGCCGTCCCCTTCTCCCTCTCCAGCCTCGGGGCTG | |
| | TCCGCGGGGGGACGGCTGCCTTCGGGGGGGACGGGGCAGGGCGGG | |
| | GTTCGGCTTCTGGCGTGTGACCGGCGGCTCTAGAGCCTCTGCTAACCA | |
| | TGTTCATGCCTTCTTCTTTTTCCTACAGCTCCTGGGCAACGTGCTGGTT | |
| | ATTGTGCTGTCTCATCATTTTGGCAAAGAATTGGAGAATAAACTAGTATT | |
| | CTTCTGGTCCCCACAGACTCAGAGAGAACCCGCCACCATGTTCGTGTT | |
| | CCTGGTGCTGCTGCCCCTGGTGAGCAGCCAGTGCGTGAACCTGACCA | |
| | CCAGGACCCAGCTGCCCCCCGCCTACACCAACAGCTTCACCAGGGGC | |
| | GTGTACTACCCCGACAAGGTGTTCAGGAGCAGCGTGCTGCACAGCACC | |
| | CAGGACCTGTTCCTGCCCTTCTTCAGCAACGTGACCTGGTTCCACGCC | |
| | ATCCACGTGAGCGGCACCAACGGCACCAAGAGGTTCGACAACCCCGT | |
| | GCTGCCCTTCAACGACGGCGTGTACTTCGCCAGCACCGAGAAGAGCAA | |
| | CATCATCAGGGGCTGGATCTTCGGCACCACCCTGGACAGCAAGACCCA | |
| | GAGCCTGCTGATCGTGAACAACGCCACCAACGTGGTGATCAAGGTGTG | |
| | CGAGTTCCAGTTCTGCAACGACCCCTTCCTGGGCGTGTACTACCACAA | |
| | GAACAACAAGAGCTGGATGGAGAGCGAGTTCAGGGTGTACAGCAGCG | |
| | CCAACAACTGCACCTTCGAGTACGTGAGCCAGCCCTTCCTGATGGACC | |
| | TGGAGGGCAAGCAGGGCAACTTCAAGAACCTGAGGGAGTTCGTGTTCA | |
| | AGAACATCGACGGCTACTTCAAGATCTACAGCAAGCACACCCCCATCAA | |
| | CCTGGTGAGGGACCTGCCCCAGGGCTTCAGCGCCCTGGAGCCCCTGG | |
| | TGGACCTGCCCATCGGCATCAACATCACCAGGTTCCAGACCCTGCTGG | |
| | CCCTGCACAGGAGCTACCTGACCCCCGGCGACAGCAGCAGCGGCTGG | |
| | ACCGCCGGCGCCGCCGCCTACTACGTGGGCTACCTGCAGCCCAGGAC | |
| | CTTCCTGCTGAAGTACAACGAGAACGGCACCATCACCGACGCCGTGGA | |
| | CTGCGCCCTGGACCCCCTGAGCGAGACCAAGTGCACCCTGAAGAGCTT | |
| | CACCGTGGAGAAGGGCATCTACCAGACCAGCAACTTCAGGGTGCAGCC | |
| | CACCGAGAGCATCGTGAGGTTCCCCAACATCACCAACCTGTGCCCCTT | |
| | CGGCGAGGTGTTCAACGCCACCAGGTTCGCCAGCGTGTACGCCTGGA | |
| | ACAGGAAGAGGATCAGCAACTGCGTGGCCGACTACAGCGTGCTGTACA | |
| | ACAGCGCCAGCTTCAGCACCTTCAAGTGCTACGGCGTGAGCCCCACCA | |
| | AGCTGAACGACCTGTGCTTCACCAACGTGTACGCCGACAGCTTCGTGA | |
| | TCAGGGGCGACGAGGTGAGGCAGATCGCCCCCGGCCAGACCGGCAA | |
| | GATCGCCGACTACAACTACAAGCTGCCCGACGACTTCACCGGCTGCGT | |
| | GATCGCCTGGAACAGCAACAACCTGGACAGCAAGGTGGGCGGCAACT | |
| | ACAACTACCTGTACAGGCTGTTCAGGAAGAGCAACCTGAAGCCCTTCG | |
| | AGAGGGACATCAGCACCGAGATCTACCAGGCCGGCAGCACCCCCTGC | |
| | AACGGCGTGGAGGGCTTCAACTGCTACTTCCCCCTGCAGAGCTACGGC | |
| | TTCCAGCCCACCAACGGCGTGGGCTACCAGCCCTACAGGGTGGTGGT | |
| | GCTGAGCTTCGAGCTGCTGCACGCCCCCGCCACCGTGTGCGGCCCCA | |
| | AGAAGAGCACCAACCTGGTGAAGAACAAGTGCGTGAACTTCAACTTCAA | |
| | CGGCCTGACCGGCACCGGCGTGCTGACCGAGAGCAACAAGAAGTTCC | |
| | TGCCCTTCCAGCAGTTCGGCAGGGACATCGCCGACACCACCGACGCC | |
| | GTGAGGGACCCCCAGACCCTGGAGATCCTGGACATCACCCCCTGCAG | |
| | CTTCGGCGGCGTGAGCGTGATCACCCCCGGCACCAACACCAGCAACC | |
| | AGGTGGCCGTGCTGTACCAGGACGTGAACTGCACCGAGGTGCCCGTG | |
| | GCCATCCACGCCGACCAGCTGACCCCCACCTGGAGGGTGTACAGCAC | |
| | CGGCAGCAACGTGTTCCAGACCAGGGCCGGCTGCCTGATCGGCGCCG | |
| | AGCACGTGAACAACAGCTACGAGTGCGACATCCCCATCGGCGCCGGC | |
| | ATCTGCGCCAGCTACCAGACCCAGACCAACAGCCCCGGCAGCGCCAG | |
| | CAGCGTGGCCAGCCAGAGCATCATCGCCTACACCATGAGCCTGGGCG | |
| | CCGAGAACAGCGTGGCCTACAGCAACAACAGCATCGCCATCCCCACCA | |
| | ACTTCACCATCAGCGTGACCACCGAGATCCTGCCCGTGAGCATGACCA | |
| | AGACCAGCGTGGACTGCACCATGTACATCTGCGGCGACAGCACCGAGT | |
| | GCAGCAACCTGCTGCTGCAGTACGGCAGCTTCTGCACCCAGCTGAACA | |
| | GGGCCCTGACCGGCATCGCCGTGGAGCAGGACAAGAACACCCAGGAG | |
| | GTGTTCGCCCAGGTGAAGCAGATCTACAAGACCCCCCCCATCAAGGAC | |
| | TTCGGCGGCTTCAACTTCAGCCAGATCCTGCCCGACCCCAGCAAGCCC | |
| | AGCAAGAGGAGCCCCATCGAGGACCTGCTGTTCAACAAGGTGACCCTG | |
| | GCCGACGCCGGCTTCATCAAGCAGTACGGCGACTGCCTGGGCGACAT | |
| | CGCCGCCAGGGACCTGATCTGCGCCCAGAAGTTCAACGGCCTGACCG | |
| | TGCTGCCCCCCCTGCTGACCGACGAGATGATCGCCCAGTACACCAGCG | |
| | CCCTGCTGGCCGGCACCATCACCAGCGGCTGGACCTTCGGCGCCGGC | |
| | CCCGCCCTGCAGATCCCCTTCCCCATGCAGATGGCCTACAGGTTCAAC | |
| | GGCATCGGCGTGACCCAGAACGTGCTGTACGAGAACCAGAAGCTGATC | |
| | GCCAACCAGTTCAACAGCGCCATCGGCAAGATCCAGGACAGCCTGAGC | |
| | AGCACCCCCAGCGCCCTGGGCAAGCTGCAGGACGTGGTGAACCAGAA | |
| | CGCCCAGGCCCTGAACACCCTGGTGAAGCAGCTGAGCAGCAACTTCG | |
| | GCGCCATCAGCAGCGTGCTGAACGACATCCTGAGCAGGCTGGACCCC | |
| | CCCGAGGCCGAGGTGCAGATCGACAGGCTGATCACCGGCAGGCTGCA | |
| | GAGCCTGCAGACCTACGTGACCCAGCAGCTGATCAGGGCCGCCGAGA | |
| | TCAGGGCCAGCGCCAACCTGGCCGCCACCAAGATGAGCGAGTGCGTG | |
| | CTGGGCCAGAGCAAGAGGGTGGACTTCTGCGGCAAGGGCTACCACCT | |
| | GATGAGCTTCCCCCAGAGCGCCCCCCACGGCGTGGTGTTCCTGCACG | |
| | TGACCTACGTGCCCGCCCAGGAGAAGAACTTCACCACCGCCCCCGCCA | |
| | TCTGCCACGACGGCAAGGCCCACTTCCCCAGGGAGGGCGTGTTCGTG | |
| | AGCAACGGCACCCACTGGTTCGTGACCCAGAGGAACTTCTACGAGCCC | |
| | CAGATCATCACCACCGACAACACCTTCGTGAGCGGCAACTGCGACGTG | |
| | GTGATCGGCATCGTGAACAACACCGTGTACGACCCCCTGCAGCCCGAG | |
| | CTGGACAGCTTCAAGGAGGAGCTGGACAAGTACTTCAAGAACCACACC | |
| | AGCCCCGACGTGGACCTGGGCGACATCAGCGGCATCAACGCCAGCGT | |
| | GGTGAACATCCAGAAGGAGATCGACAGGCTGAACGAGGTGGCCAAGA | |
| | ACCTGAACGAGAGCCTGATCGACCTGCAGGAGCTGGGCAAGTACGAG | |
| | CAGTACATCAAGTGGCCCTGGTACATCTGGCTGGGCTTCATCGCCGGC | |
| | CTGATCGCCATCGTGATGGTGACCATCATGCTGTGCTGCATGACCAGC | |
| | TGCTGCAGCTGCCTGAAGGGCTGCTGCAGCTGCGGCAGCTGCTGCAA | |
| | GTTCGACGAGGACGACAGCGAGCCCGTGCTGAAGGGCGTGAAGCTGC | |
| | ACTACACCGGAAGCGGAGCCACGAACTTCTCTCTGTTAAAGCAAGCAG | |
| | GAGATGTTGAAGAAAACCCCGGGCCTATGAGCGACAACGGCCCCCAG | |
| | AACCAGAGGAACGCCCCCAGGATCACCTTCGGCGGCCCCAGCGACA | |
| | GCACCGGCAGCAACCAGAACGGCGAGAGGAGCGGCGCCAGGAGCA | |
| | AGCAGAGGAGGCCCCAGGGCCTGCCCAACAACACCGCCAGCTGGTT | |
| | CACCGCCCTGACCCAGCACGGCAAGGAGGACCTGAAGTTCCCCAGG | |
| | GGCCAGGGCGTGCCCATCAACACCAACAGCAGCCCCGACGACCAGA | |
| | TCGGCTACTACAGGAGGGCCACCAGGAGGATCAGGGGCGGCGACGG | |
| | CAAGATGAAGGACCTGAGCCCCAGGTGGTACTTCTACTACCTGGGCA | |
| | CCGGCCCCGAGGCCGGCCTGCCCTACGGCGCCAACAAGGACGGCAT | |
| | CATCTGGGTGGCCACCGAGGGCGCCCTGAACACCCCCAAGGACCAC | |
| | ATCGGCACCAGGAACCCCGCCAACAACGCCGCCATCGTGCTGCAGCT | |
| | GCCCCAGGGCACCACCCTGCCCAAGGGCTTCTACGCCGAGGGCAGC | |
| | AGGGGCGGCAGCCAGGCCAGCAGCAGGAGCAGCAGCAGGAGCAGG | |
| | AACAGCAGCAGGAACAGCACCCCCGGCAGCAGCAGGGGCACCAGCC | |
| | CCGCCAGGATGGCCGGCAACGGCGGCGACGCCGCCCTGGCCCTGCT | |
| | GCTGCTGGACAGGCTGAACCAGCTGGAGAGCAAGATGAGCGGCAAG | |
| | GGCCAGCAGCAGCAGGGCCAGACCGTGACCAAGAAGAGCGCCGCCG | |
| | AGGCCAGCAAGAAGCCCAGGCAGAAGAGGACCGCCACCAAGGCCTA | |
| | CAACGTGACCCAGGCCTTCGGCAGGAGGGGCCCCGAGCAGACCCAG | |
| | GGCAACTTCGGCGACCAGGAGCTGATCAGGCAGGGCACCGACTACA | |
| | AGCACTGGCCCCAGATCGCCCAGTTCGCCCCCAGCGCCAGCGCCTTC | |
| | TTCGGCATGAGCAGGATCGGCATGGAGGTGACCCCCAGCGGCACCT | |
| | GGCTGACCTACACCGGCGCCATCAAGCTGGACGACAAGGACCCCAA | |
| | CTTCAAGGACCAGGTGATCCTGCTGAACAAGCACATCGACGCCTACA | |
| | AGACCTTCCCCCCCACCGAGCCCAAGAAGGACAAGAAGAAGAAGGC | |
| | CGACGAGACCCAGGCCCTGCCCCAGAGGCAGAAGAAGCAGCAGACC | |
| | GTGACCCTGCTGCCCGCCGCCGACCTGGACGACTTCAGCAAGCAGCT | |
| | 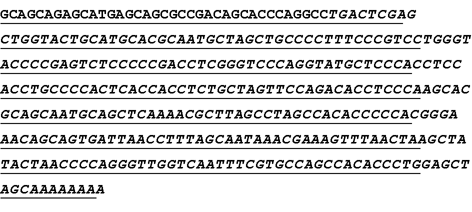 | |
| 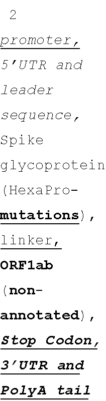 | CTCGACATTGATTATTGACTAGTTATTAATAGTAATCAATTACGGGGTCA TTAGTTCATAGCCCATATATGGAGTTCCGCGTTACATAACTTACGGTAAA TGGCCCGCCTGGCTGACCGCCCAACGACCCCCGCCCATTGACGTCAAT AATGACGTATGTTCCCATAGTAACGCCAATAGGGACTTTCCATTGACGT CAATGGGTGGAGTATTTACGGTAAACTGCCCACTTGGCAGTACATCAAG TGTATCATATGCCAAGTACGCCCCCTATTGACGTCAATGACGGTAAATG GCCCGCCTGGCATTATGCCCAGTACATGACCTTATGGGACTTTCCTACT TGGCAGTACATCTACGTATTAGTCATCGCTATTACCATGGTCGAGGTGA GCCCCACGTTCTGCTTCACTCTCCCCATCTCCCCCCCCTCCCCACCCC CAATTTTGTATTTATTTATTTTTTAATTATTTTGTGCAGCGATGGGGGCG GGGGGGGGGGGGGGGCGCGCGCCAGGCGGGGCGGGGCGGGGCGA GGGGCGGGGCGGGGCGAGGCGGAGAGGTGCGGCGGCAGCCAATCA GAGCGGCGCGCTCCGAAAGTTTCCTTTTATGGCGAGGCGGCGGCGGC GGCGGGCCTATAAAAAGCGAAGCGCGCGGCGGGCGGGAGTCGCTGC GCGCTGCCTTCGCCCCGTGCCCCGCTCCGCCGCCGCCTCGCGCCGCC CGCCCCGGCTCTGACTGACCGCGTTACTCCCACAGGTGAGCGGGCGG GACGGCCCTTCTCCTCCGGGCTGTAATTAGCGCTTGGTTTAATGACGG CTTGTTTCTTTTCTGTGGCTGCGTGAAAGCCTTGAGGGGCTCCGGGAG GGCCCTTTGTGCGGGGGGAGCGGCTCGGGGGGTGCGTGCGTGTGTG TGTGCGTGGGGAGCGCCGCGTGCGGCTCCGCGCTGCCCGGCGGCTG TGAGCGCTGCGGGCGCGGCGCGGGGCTTTGTGCGCTCCGCAGTGTGC GCGAGGGGAGCGCGGCCGGGGGCGGTGCCCCGCGGTGCGGGGGGG GCTGCGAGGGGAACAAAGGCTGCGTGCGGGGTGTGTGCGTGGGGGG GTGAGCAGGGGGTGTGGGCGCGTCGGTCGGGCTGCAACCCCCCCTG CACCCCCCTCCCCGAGTTGCTGAGCACGGCCCGGCTTCGGGTGCGGG GCTCCGTACGGGGCGTGGCGCGGGGCTCGCCGTGCCGGGCGGGGGG | 140 |
| | TGGCGGCAGGTGGGGGTGCCGGGCGGGGCGGGGCCGCCTCGGGCC | |
| | GGGGAGGGCTCGGGGGAGGGGCGCGGCGGCCCCCGGAGCGCCGGC | |
| | GGCTGTCGAGGCGCGGCGAGCCGCAGCCATTGCCTTTTATGGTAATCG | |
| | TGCGAGAGGGCGCAGGGACTTCCTTTGTCCCAAATCTGTGCGGAGCCG | |
| | AAATCTGGGAGGCGCCGCCGCACCCCCTCTAGCGGGCGCGGGGCGAA | |
| | GCGGTGCGGCGCCGGCAGGAAGGAAATGGGCGGGGAGGGCCTTCGT | |
| | GCGTCGCCGCGCCGCCGTCCCCTTCTCCCTCTCCAGCCTCGGGGCTG | |
| | TCCGCGGGGGGACGGCTGCCTTCGGGGGGGACGGGGCAGGGCGGG | |
| | GTTCGGCTTCTGGCGTGTGACCGGCGGCTCTAGAGCCTCTGCTAACCA | |
| | TGTTCATGCCTTCTTCTTTTTCCTACAGCTCCTGGGCAACGTGCTGGTT | |
| | ATTGTGCTGTCTCATCATTTTGGCAAAGAATTGGAGAATAAACTAGTATT | |
| | CTTCTGGTCCCCACAGACTCAGAGAGAACCCGCCACCATGTTCGTGTT | |
| | CCTGGTGCTGCTGCCCCTGGTGAGCAGCCAGTGCGTGAACCTGACCA | |
| | CCAGGACCCAGCTGCCCCCCGCCTACACCAACAGCTTCACCAGGGGC | |
| | GTGTACTACCCCGACAAGGTGTTCAGGAGCAGCGTGCTGCACAGCACC | |
| | CAGGACCTGTTCCTGCCCTTCTTCAGCAACGTGACCTGGTTCCACGCC | |
| | ATCCACGTGAGCGGCACCAACGGCACCAAGAGGTTCGACAACCCCGT | |
| | GCTGCCCTTCAACGACGGCGTGTACTTCGCCAGCACCGAGAAGAGCAA | |
| | CATCATCAGGGGCTGGATCTTCGGCACCACCCTGGACAGCAAGACCCA | |
| | GAGCCTGCTGATCGTGAACAACGCCACCAACGTGGTGATCAAGGTGTG | |
| | CGAGTTCCAGTTCTGCAACGACCCCTTCCTGGGCGTGTACTACCACAA | |
| | GAACAACAAGAGCTGGATGGAGAGCGAGTTCAGGGTGTACAGCAGCG | |
| | CCAACAACTGCACCTTCGAGTACGTGAGCCAGCCCTTCCTGATGGACC | |
| | TGGAGGGCAAGCAGGGCAACTTCAAGAACCTGAGGGAGTTCGTGTTCA | |
| | AGAACATCGACGGCTACTTCAAGATCTACAGCAAGCACACCCCCATCAA | |
| | CCTGGTGAGGGACCTGCCCCAGGGCTTCAGCGCCCTGGAGCCCCTGG | |
| | TGGACCTGCCCATCGGCATCAACATCACCAGGTTCCAGACCCTGCTGG | |
| | CCCTGCACAGGAGCTACCTGACCCCCGGCGACAGCAGCAGCGGCTGG | |
| | ACCGCCGGCGCCGCCGCCTACTACGTGGGCTACCTGCAGCCCAGGAC | |
| | CTTCCTGCTGAAGTACAACGAGAACGGCACCATCACCGACGCCGTGGA | |
| | CTGCGCCCTGGACCCCCTGAGCGAGACCAAGTGCACCCTGAAGAGCTT | |
| | CACCGTGGAGAAGGGCATCTACCAGACCAGCAACTTCAGGGTGCAGCC | |
| | CACCGAGAGCATCGTGAGGTTCCCCAACATCACCAACCTGTGCCCCTT | |
| | CGGCGAGGTGTTCAACGCCACCAGGTTCGCCAGCGTGTACGCCTGGA | |
| | ACAGGAAGAGGATCAGCAACTGCGTGGCCGACTACAGCGTGCTGTACA | |
| | ACAGCGCCAGCTTCAGCACCTTCAAGTGCTACGGCGTGAGCCCCACCA | |
| | AGCTGAACGACCTGTGCTTCACCAACGTGTACGCCGACAGCTTCGTGA | |
| | TCAGGGGCGACGAGGTGAGGCAGATCGCCCCCGGCCAGACCGGCAA | |
| | GATCGCCGACTACAACTACAAGCTGCCCGACGACTTCACCGGCTGCGT | |
| | GATCGCCTGGAACAGCAACAACCTGGACAGCAAGGTGGGCGGCAACT | |
| | ACAACTACCTGTACAGGCTGTTCAGGAAGAGCAACCTGAAGCCCTTCG | |
| | AGAGGGACATCAGCACCGAGATCTACCAGGCCGGCAGCACCCCCTGC | |
| | AACGGCGTGGAGGGCTTCAACTGCTACTTCCCCCTGCAGAGCTACGGC | |
| | TTCCAGCCCACCAACGGCGTGGGCTACCAGCCCTACAGGGTGGTGGT | |
| | GCTGAGCTTCGAGCTGCTGCACGCCCCCGCCACCGTGTGCGGCCCCA | |
| | AGAAGAGCACCAACCTGGTGAAGAACAAGTGCGTGAACTTCAACTTCAA | |
| | CGGCCTGACCGGCACCGGCGTGCTGACCGAGAGCAACAAGAAGTTCC | |
| | TGCCCTTCCAGCAGTTCGGCAGGGACATCGCCGACACCACCGACGCC | |
| | GTGAGGGACCCCCAGACCCTGGAGATCCTGGACATCACCCCCTGCAG | |
| | CTTCGGCGGCGTGAGCGTGATCACCCCCGGCACCAACACCAGCAACC | |
| | AGGTGGCCGTGCTGTACCAGGACGTGAACTGCACCGAGGTGCCCGTG | |
| | GCCATCCACGCCGACCAGCTGACCCCCACCTGGAGGGTGTACAGCAC | |
| | CGGCAGCAACGTGTTCCAGACCAGGGCCGGCTGCCTGATCGGCGCCG | |
| | AGCACGTGAACAACAGCTACGAGTGCGACATCCCCATCGGCGCCGGC | |
| | ATCTGCGCCAGCTACCAGACCCAGACCAACAGCCCCGGCAGCGCCAG | |
| | CAGCGTGGCCAGCCAGAGCATCATCGCCTACACCATGAGCCTGGGCG | |
| | CCGAGAACAGCGTGGCCTACAGCAACAACAGCATCGCCATCCCCACCA | |
| | ACTTCACCATCAGCGTGACCACCGAGATCCTGCCCGTGAGCATGACCA | |
| | AGACCAGCGTGGACTGCACCATGTACATCTGCGGCGACAGCACCGAGT | |
| | GCAGCAACCTGCTGCTGCAGTACGGCAGCTTCTGCACCCAGCTGAACA | |
| | GGGCCCTGACCGGCATCGCCGTGGAGCAGGACAAGAACACCCAGGAG | |
| | GTGTTCGCCCAGGTGAAGCAGATCTACAAGACCCCCCCCATCAAGGAC | |
| | TTCGGCGGCTTCAACTTCAGCCAGATCCTGCCCGACCCCAGCAAGCCC | |
| | AGCAAGAGGAGCCCCATCGAGGACCTGCTGTTCAACAAGGTGACCCTG | |
| | GCCGACGCCGGCTTCATCAAGCAGTACGGCGACTGCCTGGGCGACAT | |
| | CGCCGCCAGGGACCTGATCTGCGCCCAGAAGTTCAACGGCCTGACCG | |
| | TGCTGCCCCCCCTGCTGACCGACGAGATGATCGCCCAGTACACCAGCG | |
| | CCCTGCTGGCCGGCACCATCACCAGCGGCTGGACCTTCGGCGCCGGC | |
| | CCCGCCCTGCAGATCCCCTTCCCCATGCAGATGGCCTACAGGTTCAAC | |
| | GGCATCGGCGTGACCCAGAACGTGCTGTACGAGAACCAGAAGCTGATC | |
| | GCCAACCAGTTCAACAGCGCCATCGGCAAGATCCAGGACAGCCTGAGC | |
| | AGCACCCCCAGCGCCCTGGGCAAGCTGCAGGACGTGGTGAACCAGAA | |
| | CGCCCAGGCCCTGAACACCCTGGTGAAGCAGCTGAGCAGCAACTTCG | |
| | GCGCCATCAGCAGCGTGCTGAACGACATCCTGAGCAGGCTGGACCCC | |
| | CCCGAGGCCGAGGTGCAGATCGACAGGCTGATCACCGGCAGGCTGCA | |
| | GAGCCTGCAGACCTACGTGACCCAGCAGCTGATCAGGGCCGCCGAGA | |
| | TCAGGGCCAGCGCCAACCTGGCCGCCACCAAGATGAGCGAGTGCGTG | |
| | CTGGGCCAGAGCAAGAGGGTGGACTTCTGCGGCAAGGGCTACCACCT | |
| | GATGAGCTTCCCCCAGAGCGCCCCCCACGGCGTGGTGTTCCTGCACG | |
| | TGACCTACGTGCCCGCCCAGGAGAAGAACTTCACCACCGCCCCCGCCA | |
| | TCTGCCACGACGGCAAGGCCCACTTCCCCAGGGAGGGCGTGTTCGTG | |
| | AGCAACGGCACCCACTGGTTCGTGACCCAGAGGAACTTCTACGAGCCC | |
| | CAGATCATCACCACCGACAACACCTTCGTGAGCGGCAACTGCGACGTG | |
| | GTGATCGGCATCGTGAACAACACCGTGTACGACCCCCTGCAGCCCGAG | |
| | CTGGACAGCTTCAAGGAGGAGCTGGACAAGTACTTCAAGAACCACACC | |
| | AGCCCCGACGTGGACCTGGGCGACATCAGCGGCATCAACGCCAGCGT | |
| | GGTGAACATCCAGAAGGAGATCGACAGGCTGAACGAGGTGGCCAAGA | |
| | ACCTGAACGAGAGCCTGATCGACCTGCAGGAGCTGGGCAAGTACGAG | |
| | CAGTACATCAAGTGGCCCTGGTACATCTGGCTGGGCTTCATCGCCGGC | |
| | CTGATCGCCATCGTGATGGTGACCATCATGCTGTGCTGCATGACCAGC | |
| | TGCTGCAGCTGCCTGAAGGGCTGCTGCAGCTGCGGCAGCTGCTGCAA | |
| | GTTCGACGAGGACGACAGCGAGCCCGTGCTGAAGGGCGTGAAGCTGC | |
| | ACTACACCGGAAGCGGAGCCACGAACTTCTCTCTGTTAAAGCAAGCAG | |
| | GAGATGTTGAAGAAAACCCCGGGCCTCAAACCACTGAAACAGCWCAC | |
| | TCTTGTAATGTTAACCGCTTTAATGTGGCTATTACAAGAGCAAAAATTG | |
| | GCATTTTGTGCATAATGTCTGACAGAGATCTTTATGACAAGCTGCAATT | |
| | CACAAGTCTAGAAGTACCGCGTCGTAACGTGGCTACATTACAAGCGG | |
| | AAAATGTAACTGGACTCTTTAAGGACTGTAGTAAGATCATAACTGGTC | |
| | TTCATCCTACACAAGCACCTACACACCTTAGTGTTGATACAAAATTCA | |
| | AGACTGAGGGACTATGTGTTGACATACCAGGCATWCCWAAGGACAT | |
| | GACCTATMGWAGACTCATCTCYATGATGGGTTTCAAAATGAATTAYCA | |
| | AGTTAATGGTTACCCTAAYATGTTYATCACCCGYGARGAAGCCATMMG | |
| | MCAYGTWCGTGCATGGATTGGCTTTGATGTAGAGGGKTGTCATGCTA | |
| | CTAGGGATGCTGTCGGTACTAACCTACCTCTCCAGTTAGGATTTTCTA | |
| | CAGGTGTTAACTTAGTAGCTGTACCAACTGGCTATGTTGACACTGAAA | |
| | ACAATACAGAATTCACCAGAGTTAATGCAAAACCTCCACCAGGTGAC | |
| | CAATTTAAACATCTTATACCACTTATGTACAAAGGTTTACCCTGGAACA | |
| | TAGTGCGTATCAAGATAGTACAAATGCTCAGTGATACACTGAAAGGAT | |
| | TATCRGACAGAGTTGTGTTTGTCCTATGGGCACATGGCTTTGAACTTA | |
| | CATCAATGAAGTACTTTGTCAAGATTGGACCTGAAAGAACGTGTTGTC | |
| | TGTGTGACAAACGTGCAACTTGTTTTTCTACTTCATCAGACAATTATGC | |
| | CTGCTGGAACCATTCTGTGGGTTTTGACTATGTCTATAATCCATTTATG | |
| | ATTGATGTCCAGCAGTGGGGTTTTACAGGTAACCTTCAGAGTAATCAC | |
| | GATCAGCATTGCCAAGTGCATGGCAACGCTCATGTGGCTAGTTGTGAT | |
| | GCTATCATGACTAGATGTTTAGCAGTCCATGAGTGCTTTGTTAAGCGC | |
| | GTTGACTGGTCTGTTGAGTACCCAATTATAGGTGATGAACTGAAGATC | |
| | AATGCCGCATGCAGAAAAGTGCAACATATGGTTGTAAAGTCTGCATTG | |
| | CTTGCTGACAAATTCCCAGTTCTTCATGACATTGGAAACCCAAAGGCT | |
| | ATCAAATGTGTCCCRCAGGCTGAAGTGGATTGGAAGTTCTATGATGCT | |
| | CAGCCCTGCAGTGACAAAGCTTATAAAATAAAAGAACTCTTCTATTCT | |
| | TATGCTACACATCATGATAAATTCATTGATGGTGTTTGTTTATTTTGGA | |
| | ATTGTAACGTTGATCGTTACCCTGCCAATGCTATTGTRTGCAGGTTCG | |
| | ACACGAGAGTCTTGTCAAATTTGAACTTGCCAGGTTGTGATGGTGGTA | |
| | GTTTGTATGTAAATAAGCATGCATTCCACACTCCAGCTTTTGATAAAAG | |
| | TGCATTTACTAATTTAAAGCAATTGCCTTTCTTTTATTACTCTGACAGTC | |
| | CCTGTGAGTCACATGGCAAGCAGGTTGTTTCTGACATTGATTATGTAC | |
| | CACTCAAATCTGCTACRTGTATAACACGATGCAATTTGGGRGGTGCTG | |
| | TTTGCAGACATCATGCAAATGAGTACCGACAGTACTTGGATGCATACA | |
| | ATATGATGATTTCTGCTGGCTTTAGCCTCTGGATTTACAAACAGTTTGA | |
| | CACTTATAACCTGTGGAACACCTTTACCAGGTTACAGAGTTTAGAAAA | |
| | TGTGGCTTACAATGTTGTTAACAAAGGACACTTCGATGGACAAGCTGG | |
| | TGAAGCACCTGTTTCCGTCATTAATAATGTTGTTTACACAAAGGTAGAT | |
| | GGTGTTGATGTAGAGATCTTTGAAAACAAGACAACACTTCCTGTTAAT | |
| | GTTGCATTTGAGCTTTGGGCTAAGCGTAACATTAAACCAGTGCCAGAG | |
| | ATTAAGATACTCAATAATTTGGGTGTCGATATCGCTGCTAATACTGTAA | |
| | TCTGGGACTACAAGAGAGAAGCACCAGCACATATGTCAACAATAGGT | |
| | GTCTGCACAATGACTGACATTGCCAAGAAACCTACTGAGAGTGCTTGT | |
| | TCCTCGCTTACTGTCTTATTTGATGGTAGAGTGGAAGGACAGGTAGAC | |
| | CTTTTTAGAAATGCCCGTAATGGTGTTTTAATAACAGAAGGTTCAGTTA | |
| | AAGGTTTAATACCTTCAAAGGGACCAGCACAAGCTAGTGTCAATGGA | |
| | GTCACATTAATTGGAGAATCAGTAAAAACACAGTTTAATTATTTTAAGA | |
| | AAGTAGATGGCATCATTCAACAGTTGCCTGAAACCTACTTTACTCAGA | |
| | GCCGAGACTTAGAGGATTTCAAGCCCAGATCACAAATGGAAACTGAC | |
| | TTTCTTGAGCTCGCTATGGATGAATTCATACAACGGTACAAGCTTGAA | |
| | GGCTATGCCTTCGAACATATCGTTTATGGAGATTTTAGTCATGGACAG | |
| | CTTGGTGGACTTCATCTAATGATTGGTCTAGCTAAGCGCTCACAAGAT | |
| | TCACCACTTAAATTAGAGGATTTTATCCCTACGGACAGTACAGTGAAA | |
| | AATTATTTCATAACAGATGCGCAAACAGGTTCATCAAAATGCGTGTGC | |
| | TCTGTTATTGATCTTCTGCTTGATGACTTTGTTGAGATAATAAAGTCAC | |
| | AAGATTTATCAGTGGTTTCAAAGGTGGTCAAAGTCACAATTGACTATG | |
| | CTGAAATTTCATTCATGTTATGGTGTAAGGATGGACATGTTGAAACCTT | |
| | TTACCCAAAATTACAAGCGAGTCAGGCGTGGCAACCAGGAGTTGCAA | |
| | TGCCTAACTTGTATAAGATGCAGAGAATGCTTCTTGAAAAATGTGACC | |
| | TTCAGAATTATGGTGAAAATGCTGTCATACCAAARGGAATAATGATGA | |
| | ATGTCGCAAAATATACTCAACTGTGTCAATATTTAAATACACTYACATT | |
| | AGCYGTGCCATATAATATGAGAGTTATCCATTTTGGTGCTGGCTCRGA | |
| | CAAAGGAGTTGCACCCGGCACAGCTGTTCTCAGACAGTGGTTGCCAA | |
| | TTGGCACACTACTTGTTGATTCAGATCTTAACGACTTCGTCTCTGACGC | |
| | TGATTCCACTCTAATTGGAGACTGTGCAACCGTACATACAGCTAACAA | |
| | ATGGGATCTCATTATTAGCGATATGTATGATCCTAAAACCAAACACGT | |
| | GACAAAGGAAAATGATTCAAAAGAAGGATTTTTCACTTACCTGTGTGG | |
| | ATTTATTAAACAAAAATTAGCCCTGGGAGGCTCTGTGGCTGTAAAGAT | |
| | AACTGAGCATTCTTGGAATGCGGATCTCTACAAGCTCATGGGACATTT | |
| | CTCATGGTGGACAGCTTTTGTTACAAATGTTAATGCATCTTCATCAGAA | |
| | GCATTTTTAATTGGAGTTAACTATCTTGGTAAGCCAAAAGAACAAATT | |
| | GATGGTTACACCATGCATGCTAACTACATTTTCTGGAGGAATACAAAC | |
| | CCGATTCAATTGTCTTCCTATTCACTTTTTGACATGAGTAAGTTCCCTC | |
| | TTAAATTAAGGGGAACAGCTGTCATGTCTTTAAAGGAGAACCAAATCA | |
| | ATGAAATGATTTATTCTCTACTTGAAAAAGGCAGACTTATCATTAGGG | |
| | AAAACAACAGAGTTGTTGTCTCAAGTGATGTTCTTGTTAATAACTAAAC | |
| | 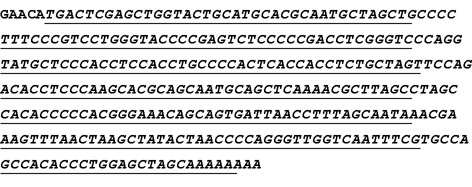 | |
| 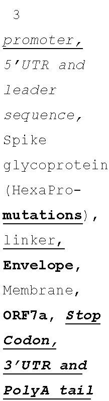 | CTCGACATTGATTATTGACTAGTTATTAATAGTAATCAATTACGGGGTCA TTAGTTCATAGCCCATATATGGAGTTCCGCGTTACATAACTTACGGTAAA TGGCCCGCCTGGCTGACCGCCCAACGACCCCCGCCCATTGACGTCAAT AATGACGTATGTTCCCATAGTAACGCCAATAGGGACTTTCCATTGACGT CAATGGGTGGAGTATTTACGGTAAACTGCCCACTTGGCAGTACATCAAG TGTATCATATGCCAAGTACGCCCCCTATTGACGTCAATGACGGTAAATG GCCCGCCTGGCATTATGCCCAGTACATGACCTTATGGGACTTTCCTACT TGGCAGTACATCTACGTATTAGTCATCGCTATTACCATGGTCGAGGTGA GCCCCACGTTCTGCTTCACTCTCCCCATCTCCCCCCCCTCCCCACCCC CAATTTTGTATTTATTTATTTTTTAATTATTTTGTGCAGCGATGGGGGCG GGGGGGGGGGGGGGGCGCGCGCCAGGCGGGGCGGGGCGGGGCGA GGGGCGGGGCGGGGCGAGGCGGAGAGGTGCGGCGGCAGCCAATCA GAGCGGCGCGCTCCGAAAGTTTCCTTTTATGGCGAGGCGGCGGCGGC GGCGGCCCTATAAAAAGCGAAGCGCGCGGCGGGCGGGAGTCGCTGC GCGCTGCCTTCGCCCCGTGCCCCGCTCCGCCGCCGCCTCGCGCCGCC CGCCCCGGCTCTGACTGACCGCGTTACTCCCACAGGTGAGCGGGCGG GACGGCCCTTCTCCTCCGGGCTGTAATTAGCGCTTGGTTTAATGACGG CTTGTTTCTTTTCTGTGGCTGCGTGAAAGCCTTGAGGGGCTCCGGGAG GGCCCTTTGTGCGGGGGGAGCGGCTCGGGGGGTGCGTGCGTGTGTG TGTGCGTGGGGAGCGCCGCGTGCGGCTCCGCGCTGCCCGGCGGCTG TGAGCGCTGCGGGCGCGGCGCGGGGCTTTGTGCGCTCCGCAGTGTGC GCGAGGGGAGCGCGGCCGGGGGCGGTGCCCCGCGGTGCGGGGGGG GCTGCGAGGGGAACAAAGGCTGCGTGCGGGGTGTGTGCGTGGGGGG GTGAGCAGGGGGTGTGGGCGCGTCGGTCGGGCTGCAACCCCCCCTG CACCCCCCTCCCCGAGTTGCTGAGCACGGCCCGGCTTCGGGTGCGGG | 141 |
| | GCTCCGTACGGGGCGTGGCGCGGGGCTCGCCGTGCCGGGCGGGGGG | |
| | TGGCGGCAGGTGGGGGTGCCGGGCGGGGCGGGGCCGCCTCGGGCC | |
| | GGGGAGGGCTCGGGGGAGGGGCGCGGCGGCCCCCGGAGCGCCGGC | |
| | GGCTGTCGAGGCGCGGCGAGCCGCAGCCATTGCCTTTTATGGTAATCG | |
| | TGCGAGAGGGCGCAGGGACTTCCTTTGTCCCAAATCTGTGCGGAGCCG | |
| | AAATCTGGGAGGCGCCGCCGCACCCCCTCTAGCGGGCGCGGGGCGAA | |
| | GCGGTGCGGCGCCGGCAGGAAGGAAATGGGCGGGGAGGGCCTTCGT | |
| | GCGTCGCCGCGCCGCCGTCCCCTTCTCCCTCTCCAGCCTCGGGGCTG | |
| | TCCGCGGGGGGACGGCTGCCTTCGGGGGGGACGGGGCAGGGCGGG | |
| | GTTCGGCTTCTGGCGTGTGACCGGCGGCTCTAGAGCCTCTGCTAACCA | |
| | TGTTCATGCCTTCTTCTTTTTCCTACAGCTCCTGGGCAACGTGCTGGTT | |
| | ATTGTGCTGTCTCATCATTTTGGCAAAGAATTGGAGAATAAACTAGTATT | |
| | CTTCTGGTCCCCACAGACTCAGAGAGAACCCGCCACCATGTTCGTGTT | |
| | CCTGGTGCTGCTGCCCCTGGTGAGCAGCCAGTGCGTGAACCTGACCA | |
| | CCAGGACCCAGCTGCCCCCCGCCTACACCAACAGCTTCACCAGGGGC | |
| | GTGTACTACCCCGACAAGGTGTTCAGGAGCAGCGTGCTGCACAGCACC | |
| | CAGGACCTGTTCCTGCCCTTCTTCAGCAACGTGACCTGGTTCCACGCC | |
| | ATCCACGTGAGCGGCACCAACGGCACCAAGAGGTTCGACAACCCCGT | |
| | GCTGCCCTTCAACGACGGCGTGTACTTCGCCAGCACCGAGAAGAGCAA | |
| | CATCATCAGGGGCTGGATCTTCGGCACCACCCTGGACAGCAAGACCCA | |
| | GAGCCTGCTGATCGTGAACAACGCCACCAACGTGGTGATCAAGGTGTG | |
| | CGAGTTCCAGTTCTGCAACGACCCCTTCCTGGGCGTGTACTACCACAA | |
| | GAACAACAAGAGCTGGATGGAGAGCGAGTTCAGGGTGTACAGCAGCG | |
| | CCAACAACTGCACCTTCGAGTACGTGAGCCAGCCCTTCCTGATGGACC | |
| | TGGAGGGCAAGCAGGGCAACTTCAAGAACCTGAGGGAGTTCGTGTTCA | |
| | AGAACATCGACGGCTACTTCAAGATCTACAGCAAGCACACCCCCATCAA | |
| | CCTGGTGAGGGACCTGCCCCAGGGCTTCAGCGCCCTGGAGCCCCTGG | |
| | TGGACCTGCCCATCGGCATCAACATCACCAGGTTCCAGACCCTGCTGG | |
| | CCCTGCACAGGAGCTACCTGACCCCCGGCGACAGCAGCAGCGGCTGG | |
| | ACCGCCGGCGCCGCCGCCTACTACGTGGGCTACCTGCAGCCCAGGAC | |
| | CTTCCTGCTGAAGTACAACGAGAACGGCACCATCACCGACGCCGTGGA | |
| | CTGCGCCCTGGACCCCCTGAGCGAGACCAAGTGCACCCTGAAGAGCTT | |
| | CACCGTGGAGAAGGGCATCTACCAGACCAGCAACTTCAGGGTGCAGCC | |
| | CACCGAGAGCATCGTGAGGTTCCCCAACATCACCAACCTGTGCCCCTT | |
| | CGGCGAGGTGTTCAACGCCACCAGGTTCGCCAGCGTGTACGCCTGGA | |
| | ACAGGAAGAGGATCAGCAACTGCGTGGCCGACTACAGCGTGCTGTACA | |
| | ACAGCGCCAGCTTCAGCACCTTCAAGTGCTACGGCGTGAGCCCCACCA | |
| | AGCTGAACGACCTGTGCTTCACCAACGTGTACGCCGACAGCTTCGTGA | |
| | TCAGGGGCGACGAGGTGAGGCAGATCGCCCCCGGCCAGACCGGCAA | |
| | GATCGCCGACTACAACTACAAGCTGCCCGACGACTTCACCGGCTGCGT | |
| | GATCGCCTGGAACAGCAACAACCTGGACAGCAAGGTGGGCGGCAACT | |
| | ACAACTACCTGTACAGGCTGTTCAGGAAGAGCAACCTGAAGCCCTTCG | |
| | AGAGGGACATCAGCACCGAGATCTACCAGGCCGGCAGCACCCCCTGC | |
| | AACGGCGTGGAGGGCTTCAACTGCTACTTCCCCCTGCAGAGCTACGGC | |
| | TTCCAGCCCACCAACGGCGTGGGCTACCAGCCCTACAGGGTGGTGGT | |
| | GCTGAGCTTCGAGCTGCTGCACGCCCCCGCCACCGTGTGCGGCCCCA | |
| | AGAAGAGCACCAACCTGGTGAAGAACAAGTGCGTGAACTTCAACTTCAA | |
| | CGGCCTGACCGGCACCGGCGTGCTGACCGAGAGCAACAAGAAGTTCC | |
| | TGCCCTTCCAGCAGTTCGGCAGGGACATCGCCGACACCACCGACGCC | |
| | GTGAGGGACCCCCAGACCCTGGAGATCCTGGACATCACCCCCTGCAG | |
| | CTTCGGCGGCGTGAGCGTGATCACCCCCGGCACCAACACCAGCAACC | |
| | AGGTGGCCGTGCTGTACCAGGACGTGAACTGCACCGAGGTGCCCGTG | |
| | GCCATCCACGCCGACCAGCTGACCCCCACCTGGAGGGTGTACAGCAC | |
| | CGGCAGCAACGTGTTCCAGACCAGGGCCGGCTGCCTGATCGGCGCCG | |
| | AGCACGTGAACAACAGCTACGAGTGCGACATCCCCATCGGCGCCGGC | |
| | ATCTGCGCCAGCTACCAGACCCAGACCAACAGCCCCGGCAGCGCCAG | |
| | CAGCGTGGCCAGCCAGAGCATCATCGCCTACACCATGAGCCTGGGCG | |
| | CCGAGAACAGCGTGGCCTACAGCAACAACAGCATCGCCATCCCCACCA | |
| | ACTTCACCATCAGCGTGACCACCGAGATCCTGCCCGTGAGCATGACCA | |
| | AGACCAGCGTGGACTGCACCATGTACATCTGCGGCGACAGCACCGAGT | |
| | GCAGCAACCTGCTGCTGCAGTACGGCAGCTTCTGCACCCAGCTGAACA | |
| | GGGCCCTGACCGGCATCGCCGTGGAGCAGGACAAGAACACCCAGGAG | |
| | GTGTTCGCCCAGGTGAAGCAGATCTACAAGACCCCCCCCATCAAGGAC | |
| | TTCGGCGGCTTCAACTTCAGCCAGATCCTGCCCGACCCCAGCAAGCCC | |
| | AGCAAGAGGAGCCCCATCGAGGACCTGCTGTTCAACAAGGTGACCCTG | |
| | GCCGACGCCGGCTTCATCAAGCAGTACGGCGACTGCCTGGGCGACAT | |
| | CGCCGCCAGGGACCTGATCTGCGCCCAGAAGTTCAACGGCCTGACCG | |
| | TGCTGCCCCCCCTGCTGACCGACGAGATGATCGCCCAGTACACCAGCG | |
| | CCCTGCTGGCCGGCACCATCACCAGCGGCTGGACCTTCGGCGCCGGC | |
| | CCCGCCCTGCAGATCCCCTTCCCCATGCAGATGGCCTACAGGTTCAAC | |
| | GGCATCGGCGTGACCCAGAACGTGCTGTACGAGAACCAGAAGCTGATC | |
| | GCCAACCAGTTCAACAGCGCCATCGGCAAGATCCAGGACAGCCTGAGC | |
| | AGCACCCCCAGCGCCCTGGGCAAGCTGCAGGACGTGGTGAACCAGAA | |
| | CGCCCAGGCCCTGAACACCCTGGTGAAGCAGCTGAGCAGCAACTTCG | |
| | GCGCCATCAGCAGCGTGCTGAACGACATCCTGAGCAGGCTGGACCCC | |
| | CCCGAGGCCGAGGTGCAGATCGACAGGCTGATCACCGGCAGGCTGCA | |
| | GAGCCTGCAGACCTACGTGACCCAGCAGCTGATCAGGGCCGCCGAGA | |
| | TCAGGGCCAGCGCCAACCTGGCCGCCACCAAGATGAGCGAGTGCGTG | |
| | CTGGGCCAGAGCAAGAGGGTGGACTTCTGCGGCAAGGGCTACCACCT | |
| | GATGAGCTTCCCCCAGAGCGCCCCCCACGGCGTGGTGTTCCTGCACG | |
| | TGACCTACGTGCCCGCCCAGGAGAAGAACTTCACCACCGCCCCCGCCA | |
| | TCTGCCACGACGGCAAGGCCCACTTCCCCAGGGAGGGCGTGTTCGTG | |
| | AGCAACGGCACCCACTGGTTCGTGACCCAGAGGAACTTCTACGAGCCC | |
| | CAGATCATCACCACCGACAACACCTTCGTGAGCGGCAACTGCGACGTG | |
| | GTGATCGGCATCGTGAACAACACCGTGTACGACCCCCTGCAGCCCGAG | |
| | CTGGACAGCTTCAAGGAGGAGCTGGACAAGTACTTCAAGAACCACACC | |
| | AGCCCCGACGTGGACCTGGGCGACATCAGCGGCATCAACGCCAGCGT | |
| | GGTGAACATCCAGAAGGAGATCGACAGGCTGAACGAGGTGGCCAAGA | |
| | ACCTGAACGAGAGCCTGATCGACCTGCAGGAGCTGGGCAAGTACGAG | |
| | CAGTACATCAAGTGGCCCTGGTACATCTGGCTGGGCTTCATCGCCGGC | |
| | CTGATCGCCATCGTGATGGTGACCATCATGCTGTGCTGCATGACCAGC | |
| | TGCTGCAGCTGCCTGAAGGGCTGCTGCAGCTGCGGCAGCTGCTGCAA | |
| | GTTCGACGAGGACGACAGCGAGCCCGTGCTGAAGGGCGTGAAGCTGC | |
| | ACTACACCGGAAGCGGAGCCACGAACTTCTCTCTGTTAAAGCAAGCAG | |
| | 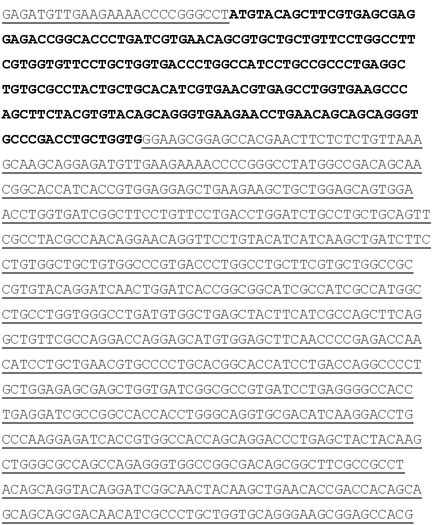 | |
| | AACTTCTCTCTGTTAAAGCAAGCAGGAGATGTTGAAGAAAACCCCGGGC | |
| | CTATGAAGATCATCCTGTTCCTGGCCCTGATCACCCTGGCCACCTGCG | |
| | 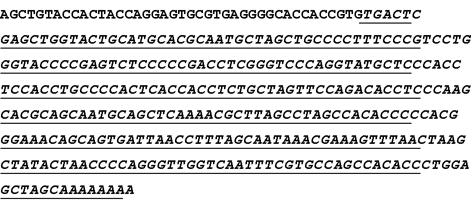 | |
| 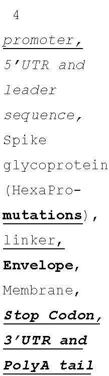 | CTCGACATTGATTATTGACTAGTTATTAATAGTAATCAATTACGGGGTCA TTAGTTCATAGCCCATATATGGAGTTCCGCGTTACATAACTTACGGTAAA TGGCCCGCCTGGCTGACCGCCCAACGACCCCCGCCCATTGACGTCAAT AATGACGTATGTTCCCATAGTAACGCCAATAGGGACTTTCCATTGACGT CAATGGGTGGAGTATTTACGGTAAACTGCCCACTTGGCAGTACATCAAG TGTATCATATGCCAAGTACGCCCCCTATTGACGTCAATGACGGTAAATG GCCCGCCTGGCATTATGCCCAGTACATGACCTTATGGGACTTTCCTACT TGGCAGTACATCTACGTATTAGTCATCGCTATTACCATGGTCGAGGTGA GCCCCACGTTCTGCTTCACTCTCCCCATCTCCCCCCCCTCCCCACCCC CAATTTTGTATTTATTTATTTTTTAATTATTTTGTGCAGCGATGGGGGCG GGGGGGGGGGGGGGGCGCGCGCCAGGCGGGGCGGGGCGGGGCGA GGGGCGGGGCGGGGCGAGGCGGAGAGGTGCGGCGGCAGCCAATCA GAGCGGCGCGCTCCGAAAGTTTCCTTTTATGGCGAGGCGGCGGCGGC GGCGGCCCTATAAAAAGCGAAGCGCGCGGCGGGCGGGAGTCGCTGC GCGCTGCCTTCGCCCCGTGCCCCGCTCCGCCGCCGCCTCGCGCCGCC CGCCCCGGCTCTGACTGACCGCGTTACTCCCACAGGTGAGCGGGCGG GACGGCCCTTCTCCTCCGGGCTGTAATTAGCGCTTGGTTTAATGACGG CTTGTTTCTTTTCTGTGGCTGCGTGAAAGCCTTGAGGGGCTCCGGGAG GGCCCTTTGTGCGGGGGGAGCGGCTCGGGGGGTGCGTGCGTGTGTG TGTGCGTGGGGAGCGCCGCGTGCGGCTCCGCGCTGCCCGGCGGCTG TGAGCGCTGCGGGCGCGGCGCGGGGCTTTGTGCGCTCCGCAGTGTGC GCGAGGGGAGCGCGGCCGGGGGCGGTGCCCCGCGGTGCGGGGGGG GCTGCGAGGGGAACAAAGGCTGCGTGCGGGGTGTGTGCGTGGGGGG GTGAGCAGGGGGTGTGGGCGCGTCGGTCGGGCTGCAACCCCCCCTG | 142 |
| | CACCCCCCTCCCCGAGTTGCTGAGCACGGCCCGGCTTCGGGTGCGGG | |
| | GCTCCGTACGGGGCGTGGCGCGGGGCTCGCCGTGCCGGGCGGGGGG | |
| | TGGCGGCAGGTGGGGGTGCCGGGCGGGGCGGGGCCGCCTCGGGCC | |
| | GGGGAGGGCTCGGGGGAGGGGCGCGGCGGCCCCCGGAGCGCCGGC | |
| | GGCTGTCGAGGCGCGGCGAGCCGCAGCCATTGCCTTTTATGGTAATCG | |
| | TGCGAGAGGGCGCAGGGACTTCCTTTGTCCCAAATCTGTGCGGAGCCG | |
| | AAATCTGGGAGGCGCCGCCGCACCCCCTCTAGCGGGCGCGGGGCGAA | |
| | GCGGTGCGGCGCCGGCAGGAAGGAAATGGGCGGGGAGGGCCTTCGT | |
| | GCGTCGCCGCGCCGCCGTCCCCTTCTCCCTCTCCAGCCTCGGGGCTG | |
| | TCCGCGGGGGGACGGCTGCCTTCGGGGGGGACGGGGCAGGGCGGG | |
| | GTTCGGCTTCTGGCGTGTGACCGGCGGCTCTAGAGCCTCTGCTAACCA | |
| | TGTTCATGCCTTCTTCTTTTTCCTACAGCTCCTGGGCAACGTGCTGGTT | |
| | ATTGTGCTGTCTCATCATTTTGGCAAAGAATTGGAGAATAAACTAGTATT | |
| | CTTCTGGTCCCCACAGACTCAGAGAGAACCCGCCACCATGTTCGTGTT | |
| | CCTGGTGCTGCTGCCCCTGGTGAGCAGCCAGTGCGTGAACCTGACCA | |
| | CCAGGACCCAGCTGCCCCCCGCCTACACCAACAGCTTCACCAGGGGC | |
| | GTGTACTACCCCGACAAGGTGTTCAGGAGCAGCGTGCTGCACAGCACC | |
| | CAGGACCTGTTCCTGCCCTTCTTCAGCAACGTGACCTGGTTCCACGCC | |
| | ATCCACGTGAGCGGCACCAACGGCACCAAGAGGTTCGACAACCCCGT | |
| | GCTGCCCTTCAACGACGGCGTGTACTTCGCCAGCACCGAGAAGAGCAA | |
| | CATCATCAGGGGCTGGATCTTCGGCACCACCCTGGACAGCAAGACCCA | |
| | GAGCCTGCTGATCGTGAACAACGCCACCAACGTGGTGATCAAGGTGTG | |
| | CGAGTTCCAGTTCTGCAACGACCCCTTCCTGGGCGTGTACTACCACAA | |
| | GAACAACAAGAGCTGGATGGAGAGCGAGTTCAGGGTGTACAGCAGCG | |
| | CCAACAACTGCACCTTCGAGTACGTGAGCCAGCCCTTCCTGATGGACC | |
| | TGGAGGGCAAGCAGGGCAACTTCAAGAACCTGAGGGAGTTCGTGTTCA | |
| | AGAACATCGACGGCTACTTCAAGATCTACAGCAAGCACACCCCCATCAA | |
| | CCTGGTGAGGGACCTGCCCCAGGGCTTCAGCGCCCTGGAGCCCCTGG | |
| | TGGACCTGCCCATCGGCATCAACATCACCAGGTTCCAGACCCTGCTGG | |
| | CCCTGCACAGGAGCTACCTGACCCCCGGCGACAGCAGCAGCGGCTGG | |
| | ACCGCCGGCGCCGCCGCCTACTACGTGGGCTACCTGCAGCCCAGGAC | |
| | CTTCCTGCTGAAGTACAACGAGAACGGCACCATCACCGACGCCGTGGA | |
| | CTGCGCCCTGGACCCCCTGAGCGAGACCAAGTGCACCCTGAAGAGCTT | |
| | CACCGTGGAGAAGGGCATCTACCAGACCAGCAACTTCAGGGTGCAGCC | |
| | CACCGAGAGCATCGTGAGGTTCCCCAACATCACCAACCTGTGCCCCTT | |
| | CGGCGAGGTGTTCAACGCCACCAGGTTCGCCAGCGTGTACGCCTGGA | |
| | ACAGGAAGAGGATCAGCAACTGCGTGGCCGACTACAGCGTGCTGTACA | |
| | ACAGCGCCAGCTTCAGCACCTTCAAGTGCTACGGCGTGAGCCCCACCA | |
| | AGCTGAACGACCTGTGCTTCACCAACGTGTACGCCGACAGCTTCGTGA | |
| | TCAGGGGCGACGAGGTGAGGCAGATCGCCCCCGGCCAGACCGGCAA | |
| | GATCGCCGACTACAACTACAAGCTGCCCGACGACTTCACCGGCTGCGT | |
| | GATCGCCTGGAACAGCAACAACCTGGACAGCAAGGTGGGCGGCAACT | |
| | ACAACTACCTGTACAGGCTGTTCAGGAAGAGCAACCTGAAGCCCTTCG | |
| | AGAGGGACATCAGCACCGAGATCTACCAGGCCGGCAGCACCCCCTGC | |
| | AACGGCGTGGAGGGCTTCAACTGCTACTTCCCCCTGCAGAGCTACGGC | |
| | TTCCAGCCCACCAACGGCGTGGGCTACCAGCCCTACAGGGTGGTGGT | |
| | GCTGAGCTTCGAGCTGCTGCACGCCCCCGCCACCGTGTGCGGCCCCA | |
| | AGAAGAGCACCAACCTGGTGAAGAACAAGTGCGTGAACTTCAACTTCAA | |
| | CGGCCTGACCGGCACCGGCGTGCTGACCGAGAGCAACAAGAAGTTCC | |
| | TGCCCTTCCAGCAGTTCGGCAGGGACATCGCCGACACCACCGACGCC | |
| | GTGAGGGACCCCCAGACCCTGGAGATCCTGGACATCACCCCCTGCAG | |
| | CTTCGGCGGCGTGAGCGTGATCACCCCCGGCACCAACACCAGCAACC | |
| | AGGTGGCCGTGCTGTACCAGGACGTGAACTGCACCGAGGTGCCCGTG | |
| | GCCATCCACGCCGACCAGCTGACCCCCACCTGGAGGGTGTACAGCAC | |
| | CGGCAGCAACGTGTTCCAGACCAGGGCCGGCTGCCTGATCGGCGCCG | |
| | AGCACGTGAACAACAGCTACGAGTGCGACATCCCCATCGGCGCCGGC | |
| | ATCTGCGCCAGCTACCAGACCCAGACCAACAGCCCCGGCAGCGCCAG | |
| | CAGCGTGGCCAGCCAGAGCATCATCGCCTACACCATGAGCCTGGGCG | |
| | CCGAGAACAGCGTGGCCTACAGCAACAACAGCATCGCCATCCCCACCA | |
| | ACTTCACCATCAGCGTGACCACCGAGATCCTGCCCGTGAGCATGACCA | |
| | AGACCAGCGTGGACTGCACCATGTACATCTGCGGCGACAGCACCGAGT | |
| | GCAGCAACCTGCTGCTGCAGTACGGCAGCTTCTGCACCCAGCTGAACA | |
| | GGGCCCTGACCGGCATCGCCGTGGAGCAGGACAAGAACACCCAGGAG | |
| | GTGTTCGCCCAGGTGAAGCAGATCTACAAGACCCCCCCCATCAAGGAC | |
| | TTCGGCGGCTTCAACTTCAGCCAGATCCTGCCCGACCCCAGCAAGCCC | |
| | AGCAAGAGGAGCCCCATCGAGGACCTGCTGTTCAACAAGGTGACCCTG | |
| | GCCGACGCCGGCTTCATCAAGCAGTACGGCGACTGCCTGGGCGACAT | |
| | CGCCGCCAGGGACCTGATCTGCGCCCAGAAGTTCAACGGCCTGACCG | |
| | TGCTGCCCCCCCTGCTGACCGACGAGATGATCGCCCAGTACACCAGCG | |
| | CCCTGCTGGCCGGCACCATCACCAGCGGCTGGACCTTCGGCGCCGGC | |
| | CCCGCCCTGCAGATCCCCTTCCCCATGCAGATGGCCTACAGGTTCAAC | |
| | GGCATCGGCGTGACCCAGAACGTGCTGTACGAGAACCAGAAGCTGATC | |
| | GCCAACCAGTTCAACAGCGCCATCGGCAAGATCCAGGACAGCCTGAGC | |
| | AGCACCCCCAGCGCCCTGGGCAAGCTGCAGGACGTGGTGAACCAGAA | |
| | CGCCCAGGCCCTGAACACCCTGGTGAAGCAGCTGAGCAGCAACTTCG | |
| | GCGCCATCAGCAGCGTGCTGAACGACATCCTGAGCAGGCTGGACCCC | |
| | CCCGAGGCCGAGGTGCAGATCGACAGGCTGATCACCGGCAGGCTGCA | |
| | GAGCCTGCAGACCTACGTGACCCAGCAGCTGATCAGGGCCGCCGAGA | |
| | TCAGGGCCAGCGCCAACCTGGCCGCCACCAAGATGAGCGAGTGCGTG | |
| | CTGGGCCAGAGCAAGAGGGTGGACTTCTGCGGCAAGGGCTACCACCT | |
| | GATGAGCTTCCCCCAGAGCGCCCCCCACGGCGTGGTGTTCCTGCACG | |
| | TGACCTACGTGCCCGCCCAGGAGAAGAACTTCACCACCGCCCCCGCCA | |
| | TCTGCCACGACGGCAAGGCCCACTTCCCCAGGGAGGGCGTGTTCGTG | |
| | AGCAACGGCACCCACTGGTTCGTGACCCAGAGGAACTTCTACGAGCCC | |
| | CAGATCATCACCACCGACAACACCTTCGTGAGCGGCAACTGCGACGTG | |
| | GTGATCGGCATCGTGAACAACACCGTGTACGACCCCCTGCAGCCCGAG | |
| | CTGGACAGCTTCAAGGAGGAGCTGGACAAGTACTTCAAGAACCACACC | |
| | AGCCCCGACGTGGACCTGGGCGACATCAGCGGCATCAACGCCAGCGT | |
| | GGTGAACATCCAGAAGGAGATCGACAGGCTGAACGAGGTGGCCAAGA | |
| | ACCTGAACGAGAGCCTGATCGACCTGCAGGAGCTGGGCAAGTACGAG | |
| | CAGTACATCAAGTGGCCCTGGTACATCTGGCTGGGCTTCATCGCCGGC | |
| | CTGATCGCCATCGTGATGGTGACCATCATGCTGTGCTGCATGACCAGC | |
| | TGCTGCAGCTGCCTGAAGGGCTGCTGCAGCTGCGGCAGCTGCTGCAA | |
| | GTTCGACGAGGACGACAGCGAGCCCGTGCTGAAGGGCGTGAAGCTGC | |
| | ACTACACCGGAAGCGGAGCCACGAACTTCTCTCTGTTAAAGCAAGCAG | |
| | 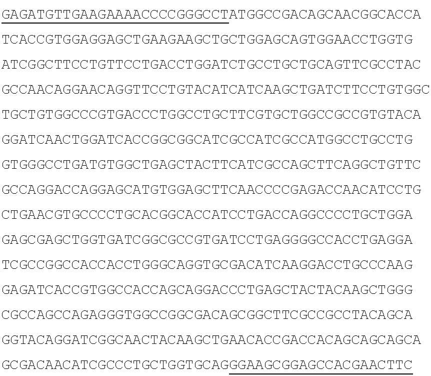 | |
| | 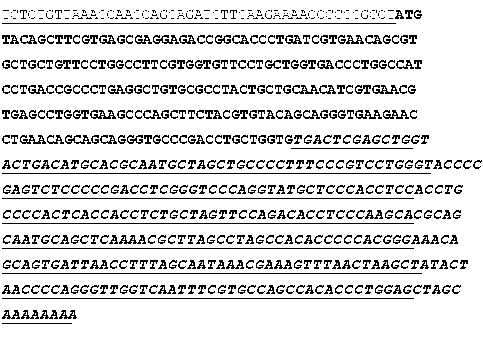 | |
| 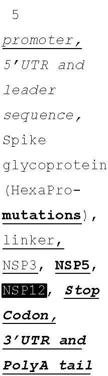 | CTCGACATTGATTATTGACTAGTTATTAATAGTAATCAATTACGGGGTCA TTAGTTCATAGCCCATATATGGAGTTCCGCGTTACATAACTTACGGTAAA TGGCCCGCCTGGCTGACCGCCCAACGACCCCCGCCCATTGACGTCAAT AATGACGTATGTTCCCATAGTAACGCCAATAGGGACTTTCCATTGACGT CAATGGGTGGAGTATTTACGGTAAACTGCCCACTTGGCAGTACATCAAG TGTATCATATGCCAAGTACGCCCCCTATTGACGTCAATGACGGTAAATG GCCCGCCTGGCATTATGCCCAGTACATGACCTTATGGGACTTTCCTACT TGGCAGTACATCTACGTATTAGTCATCGCTATTACCATGGTCGAGGTGA GCCCCACGTTCTGCTTCACTCTCCCCATCTCCCCCCCCTCCCCACCCC CAATTTTGTATTTATTTATTTTTTAATTATTTTGTGCAGCGATGGGGGCG GGGGGGGGGGGGGGGCGCGCGCCAGGCGGGGCGGGGCGGGGCGA GGGGCGGGGCGGGGCGAGGCGGAGAGGTGCGGCGGCAGCCAATCA GAGCGGCGCGCTCCGAAAGTTTCCTTTTATGGCGAGGCGGCGGCGGC GGCGGCCCTATAAAAAGCGAAGCGCGCGGCGGGCGGGAGTCGCTGC GCGCTGCCTTCGCCCCGTGCCCCGCTCCGCCGCCGCCTCGCGCCGCC CGCCCCGGCTCTGACTGACCGCGTTACTCCCACAGGTGAGCGGGCGG GACGGCCCTTCTCCTCCGGGCTGTAATTAGCGCTTGGTTTAATGACGG CTTGTTTCTTTTCTGTGGCTGCGTGAAAGCCTTGAGGGGCTCCGGGAG GGCCCTTTGTGCGGGGGGAGCGGCTCGGGGGGTGCGTGCGTGTGTG TGTGCGTGGGGAGCGCCGCGTGCGGCTCCGCGCTGCCCGGCGGCTG TGAGCGCTGCGGGCGCGGCGCGGGGCTTTGTGCGCTCCGCAGTGTGC GCGAGGGGAGCGCGGCCGGGGGCGGTGCCCCGCGGTGCGGGGGGG GCTGCGAGGGGAACAAAGGCTGCGTGCGGGGTGTGTGCGTGGGGGG GTGAGCAGGGGGTGTGGGCGCGTCGGTCGGGCTGCAACCCCCCCTG | 143 |
| | CACCCCCCTCCCCGAGTTGCTGAGCACGGCCCGGCTTCGGGTGCGGG | |
| | GCTCCGTACGGGGCGTGGCGCGGGGCTCGCCGTGCCGGGCGGGGGG | |
| | TGGCGGCAGGTGGGGGTGCCGGGCGGGGCGGGGCCGCCTCGGGCC | |
| | GGGGAGGGCTCGGGGGAGGGGCGCGGCGGCCCCCGGAGCGCCGGC | |
| | GGCTGTCGAGGCGCGGCGAGCCGCAGCCATTGCCTTTTATGGTAATCG | |
| | TGCGAGAGGGCGCAGGGACTTCCTTTGTCCCAAATCTGTGCGGAGCCG | |
| | AAATCTGGGAGGCGCCGCCGCACCCCCTCTAGCGGGCGCGGGGCGAA | |
| | GCGGTGCGGCGCCGGCAGGAAGGAAATGGGCGGGGAGGGCCTTCGT | |
| | GCGTCGCCGCGCCGCCGTCCCCTTCTCCCTCTCCAGCCTCGGGGCTG | |
| | TCCGCGGGGGGACGGCTGCCTTCGGGGGGGACGGGGCAGGGCGGG | |
| | GTTCGGCTTCTGGCGTGTGACCGGCGGCTCTAGAGCCTCTGCTAACCA | |
| | TGTTCATGCCTTCTTCTTTTTCCTACAGCTCCTGGGCAACGTGCTGGTT | |
| | ATTGTGCTGTCTCATCATTTTGGCAAAGAATTGGAGAATAAACTAGTATT | |
| | CTTCTGGTCCCCACAGACTCAGAGAGAACCCGCCACCATGTTCGTGTT | |
| | CCTGGTGCTGCTGCCCCTGGTGAGCAGCCAGTGCGTGAACCTGACCA | |
| | CCAGGACCCAGCTGCCCCCCGCCTACACCAACAGCTTCACCAGGGGC | |
| | GTGTACTACCCCGACAAGGTGTTCAGGAGCAGCGTGCTGCACAGCACC | |
| | CAGGACCTGTTCCTGCCCTTCTTCAGCAACGTGACCTGGTTCCACGCC | |
| | ATCCACGTGAGCGGCACCAACGGCACCAAGAGGTTCGACAACCCCGT | |
| | GCTGCCCTTCAACGACGGCGTGTACTTCGCCAGCACCGAGAAGAGCAA | |
| | CATCATCAGGGGCTGGATCTTCGGCACCACCCTGGACAGCAAGACCCA | |
| | GAGCCTGCTGATCGTGAACAACGCCACCAACGTGGTGATCAAGGTGTG | |
| | CGAGTTCCAGTTCTGCAACGACCCCTTCCTGGGCGTGTACTACCACAA | |
| | GAACAACAAGAGCTGGATGGAGAGCGAGTTCAGGGTGTACAGCAGCG | |
| | CCAACAACTGCACCTTCGAGTACGTGAGCCAGCCCTTCCTGATGGACC | |
| | TGGAGGGCAAGCAGGGCAACTTCAAGAACCTGAGGGAGTTCGTGTTCA | |
| | AGAACATCGACGGCTACTTCAAGATCTACAGCAAGCACACCCCCATCAA | |
| | CCTGGTGAGGGACCTGCCCCAGGGCTTCAGCGCCCTGGAGCCCCTGG | |
| | TGGACCTGCCCATCGGCATCAACATCACCAGGTTCCAGACCCTGCTGG | |
| | CCCTGCACAGGAGCTACCTGACCCCCGGCGACAGCAGCAGCGGCTGG | |
| | ACCGCCGGCGCCGCCGCCTACTACGTGGGCTACCTGCAGCCCAGGAC | |
| | CTTCCTGCTGAAGTACAACGAGAACGGCACCATCACCGACGCCGTGGA | |
| | CTGCGCCCTGGACCCCCTGAGCGAGACCAAGTGCACCCTGAAGAGCTT | |
| | CACCGTGGAGAAGGGCATCTACCAGACCAGCAACTTCAGGGTGCAGCC | |
| | CACCGAGAGCATCGTGAGGTTCCCCAACATCACCAACCTGTGCCCCTT | |
| | CGGCGAGGTGTTCAACGCCACCAGGTTCGCCAGCGTGTACGCCTGGA | |
| | ACAGGAAGAGGATCAGCAACTGCGTGGCCGACTACAGCGTGCTGTACA | |
| | ACAGCGCCAGCTTCAGCACCTTCAAGTGCTACGGCGTGAGCCCCACCA | |
| | AGCTGAACGACCTGTGCTTCACCAACGTGTACGCCGACAGCTTCGTGA | |
| | TCAGGGGCGACGAGGTGAGGCAGATCGCCCCCGGCCAGACCGGCAA | |
| | GATCGCCGACTACAACTACAAGCTGCCCGACGACTTCACCGGCTGCGT | |
| | GATCGCCTGGAACAGCAACAACCTGGACAGCAAGGTGGGCGGCAACT | |
| | ACAACTACCTGTACAGGCTGTTCAGGAAGAGCAACCTGAAGCCCTTCG | |
| | AGAGGGACATCAGCACCGAGATCTACCAGGCCGGCAGCACCCCCTGC | |
| | AACGGCGTGGAGGGCTTCAACTGCTACTTCCCCCTGCAGAGCTACGGC | |
| | TTCCAGCCCACCAACGGCGTGGGCTACCAGCCCTACAGGGTGGTGGT | |
| | GCTGAGCTTCGAGCTGCTGCACGCCCCCGCCACCGTGTGCGGCCCCA | |
| | AGAAGAGCACCAACCTGGTGAAGAACAAGTGCGTGAACTTCAACTTCAA | |
| | CGGCCTGACCGGCACCGGCGTGCTGACCGAGAGCAACAAGAAGTTCC | |
| | TGCCCTTCCAGCAGTTCGGCAGGGACATCGCCGACACCACCGACGCC | |
| | GTGAGGGACCCCCAGACCCTGGAGATCCTGGACATCACCCCCTGCAG | |
| | CTTCGGCGGCGTGAGCGTGATCACCCCCGGCACCAACACCAGCAACC | |
| | AGGTGGCCGTGCTGTACCAGGACGTGAACTGCACCGAGGTGCCCGTG | |
| | GCCATCCACGCCGACCAGCTGACCCCCACCTGGAGGGTGTACAGCAC | |
| | CGGCAGCAACGTGTTCCAGACCAGGGCCGGCTGCCTGATCGGCGCCG | |
| | AGCACGTGAACAACAGCTACGAGTGCGACATCCCCATCGGCGCCGGC | |
| | ATCTGCGCCAGCTACCAGACCCAGACCAACAGCCCCGGCAGCGCCAG | |
| | CAGCGTGGCCAGCCAGAGCATCATCGCCTACACCATGAGCCTGGGCG | |
| | CCGAGAACAGCGTGGCCTACAGCAACAACAGCATCGCCATCCCCACCA | |
| | ACTTCACCATCAGCGTGACCACCGAGATCCTGCCCGTGAGCATGACCA | |
| | AGACCAGCGTGGACTGCACCATGTACATCTGCGGCGACAGCACCGAGT | |
| | GCAGCAACCTGCTGCTGCAGTACGGCAGCTTCTGCACCCAGCTGAACA | |
| | GGGCCCTGACCGGCATCGCCGTGGAGCAGGACAAGAACACCCAGGAG | |
| | GTGTTCGCCCAGGTGAAGCAGATCTACAAGACCCCCCCCATCAAGGAC | |
| | TTCGGCGGCTTCAACTTCAGCCAGATCCTGCCCGACCCCAGCAAGCCC | |
| | AGCAAGAGGAGCCCCATCGAGGACCTGCTGTTCAACAAGGTGACCCTG | |
| | GCCGACGCCGGCTTCATCAAGCAGTACGGCGACTGCCTGGGCGACAT | |
| | CGCCGCCAGGGACCTGATCTGCGCCCAGAAGTTCAACGGCCTGACCG | |
| | TGCTGCCCCCCCTGCTGACCGACGAGATGATCGCCCAGTACACCAGCG | |
| | CCCTGCTGGCCGGCACCATCACCAGCGGCTGGACCTTCGGCGCCGGC | |
| | CCCGCCCTGCAGATCCCCTTCCCCATGCAGATGGCCTACAGGTTCAAC | |
| | GGCATCGGCGTGACCCAGAACGTGCTGTACGAGAACCAGAAGCTGATC | |
| | GCCAACCAGTTCAACAGCGCCATCGGCAAGATCCAGGACAGCCTGAGC | |
| | AGCACCCCCAGCGCCCTGGGCAAGCTGCAGGACGTGGTGAACCAGAA | |
| | CGCCCAGGCCCTGAACACCCTGGTGAAGCAGCTGAGCAGCAACTTCG | |
| | GCGCCATCAGCAGCGTGCTGAACGACATCCTGAGCAGGCTGGACCCC | |
| | CCCGAGGCCGAGGTGCAGATCGACAGGCTGATCACCGGCAGGCTGCA | |
| | GAGCCTGCAGACCTACGTGACCCAGCAGCTGATCAGGGCCGCCGAGA | |
| | TCAGGGCCAGCGCCAACCTGGCCGCCACCAAGATGAGCGAGTGCGTG | |
| | CTGGGCCAGAGCAAGAGGGTGGACTTCTGCGGCAAGGGCTACCACCT | |
| | GATGAGCTTCCCCCAGAGCGCCCCCCACGGCGTGGTGTTCCTGCACG | |
| | TGACCTACGTGCCCGCCCAGGAGAAGAACTTCACCACCGCCCCCGCCA | |
| | TCTGCCACGACGGCAAGGCCCACTTCCCCAGGGAGGGCGTGTTCGTG | |
| | AGCAACGGCACCCACTGGTTCGTGACCCAGAGGAACTTCTACGAGCCC | |
| | CAGATCATCACCACCGACAACACCTTCGTGAGCGGCAACTGCGACGTG | |
| | GTGATCGGCATCGTGAACAACACCGTGTACGACCCCCTGCAGCCCGAG | |
| | CTGGACAGCTTCAAGGAGGAGCTGGACAAGTACTTCAAGAACCACACC | |
| | AGCCCCGACGTGGACCTGGGCGACATCAGCGGCATCAACGCCAGCGT | |
| | GGTGAACATCCAGAAGGAGATCGACAGGCTGAACGAGGTGGCCAAGA | |
| | ACCTGAACGAGAGCCTGATCGACCTGCAGGAGCTGGGCAAGTACGAG | |
| | CAGTACATCAAGTGGCCCTGGTACATCTGGCTGGGCTTCATCGCCGGC | |
| | CTGATCGCCATCGTGATGGTGACCATCATGCTGTGCTGCATGACCAGC | |
| | TGCTGCAGCTGCCTGAAGGGCTGCTGCAGCTGCGGCAGCTGCTGCAA | |
| | GTTCGACGAGGACGACAGCGAGCCCGTGCTGAAGGGCGTGAAGCTGC | |
| | ACTACACCGGAAGCGGAGCCACGAACTTCTCTCTGTTAAAGCAAGCAG | |
| | 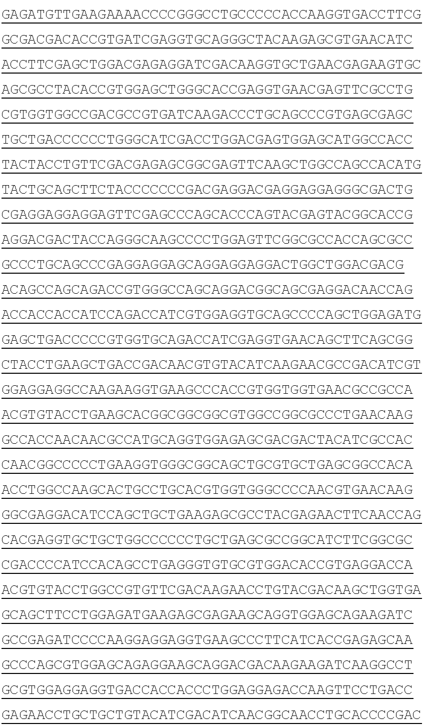 | |
| | 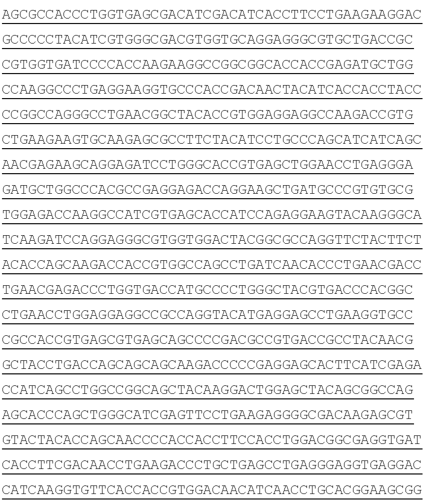 | |
| | AGCCACGAACTTCTCTCTGTTAAAGCAAGCAGGAGATGTTGAAGAAAAC | |
| | 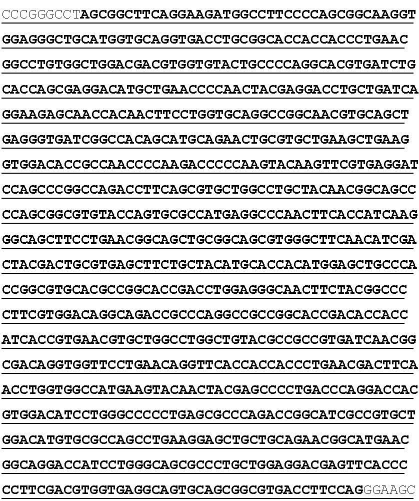 | |
| | GGAGCCACGAACTTCTCTCTGTTAAAGCAAGCAGGAGATGTTGAAGAAA | |
| | 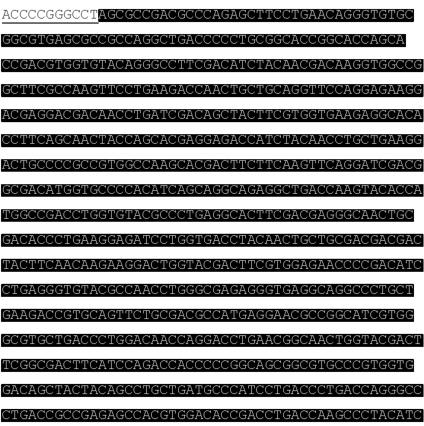 | |
| | 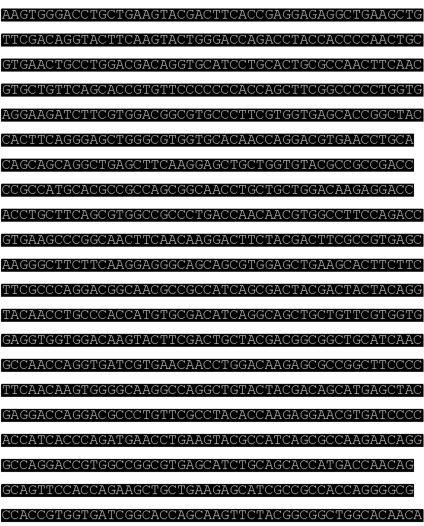 | |
| | 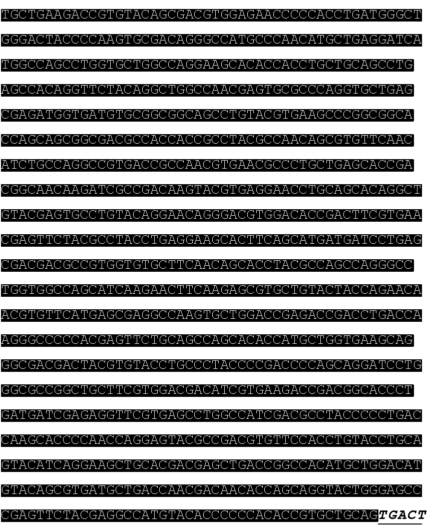 | |
| | 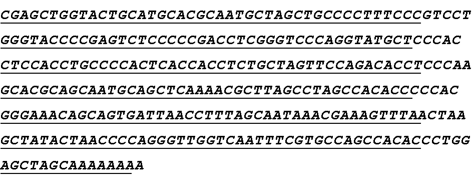 | |
| 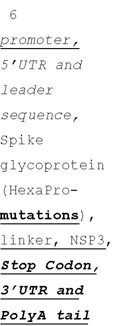 | CTCGACATTGATTATTGACTAGTTATTAATAGTAATCAATTACGGGGTCA TTAGTTCATAGCCCATATATGGAGTTCCGCGTTACATAACTTACGGTAAA TGGCCCGCCTGGCTGACCGCCCAACGACCCCCGCCCATTGACGTCAAT AATGACGTATGTTCCCATAGTAACGCCAATAGGGACTTTCCATTGACGT CAATGGGTGGAGTATTTACGGTAAACTGCCCACTTGGCAGTACATCAAG TGTATCATATGCCAAGTACGCCCCCTATTGACGTCAATGACGGTAAATG GCCCGCCTGGCATTATGCCCAGTACATGACCTTATGGGACTTTCCTACT TGGCAGTACATCTACGTATTAGTCATCGCTATTACCATGGTCGAGGTGA GCCCCACGTTCTGCTTCACTCTCCCCATCTCCCCCCCCTCCCCACCCC CAATTTTGTATTTATTTATTTTTTAATTATTTTGTGCAGCGATGGGGGCG GGGGGGGGGGGGGGGCGCGCGCCAGGCGGGGCGGGGCGGGGCGA GGGGCGGGGCGGGGCGAGGCGGAGAGGTGCGGCGGCAGCCAATCA GAGCGGCGCGCTCCGAAAGTTTCCTTTTATGGCGAGGCGGCGGCGGC GGCGGCCCTATAAAAAGCGAAGCGCGCGGCGGGCGGGAGTCGCTGC GCGCTGCCTTCGCCCCGTGCCCCGCTCCGCCGCCGCCTCGCGCCGCC CGCCCCGGCTCTGACTGACCGCGTTACTCCCACAGGTGAGCGGGCGG GACGGCCCTTCTCCTCCGGGCTGTAATTAGCGCTTGGTTTAATGACGG CTTGTTTCTTTTCTGTGGCTGCGTGAAAGCCTTGAGGGGCTCCGGGAG GGCCCTTTGTGCGGGGGGAGCGGCTCGGGGGGTGCGTGCGTGTGTG TGTGCGTGGGGAGCGCCGCGTGCGGCTCCGCGCTGCCCGGCGGCTG TGAGCGCTGCGGGCGCGGCGCGGGGCTTTGTGCGCTCCGCAGTGTGC | 144 |
| | GCGAGGGGAGCGCGGCCGGGGGCGGTGCCCCGCGGTGCGGGGGGG | |
| | GCTGCGAGGGGAACAAAGGCTGCGTGCGGGGTGTGTGCGTGGGGGG | |
| | GTGAGCAGGGGGTGTGGGCGCGTCGGTCGGGCTGCAACCCCCCCTG | |
| | CACCCCCCTCCCCGAGTTGCTGAGCACGGCCCGGCTTCGGGTGCGGG | |
| | GCTCCGTACGGGGCGTGGCGCGGGGCTCGCCGTGCCGGGCGGGGGG | |
| | TGGCGGCAGGTGGGGGTGCCGGGCGGGGCGGGGCCGCCTCGGGCC | |
| | GGGGAGGGCTCGGGGGAGGGGCGCGGCGGCCCCCGGAGCGCCGGC | |
| | GGCTGTCGAGGCGCGGCGAGCCGCAGCCATTGCCTTTTATGGTAATCG | |
| | TGCGAGAGGGCGCAGGGACTTCCTTTGTCCCAAATCTGTGCGGAGCCG | |
| | AAATCTGGGAGGCGCCGCCGCACCCCCTCTAGCGGGCGCGGGGCGAA | |
| | GCGGTGCGGCGCCGGCAGGAAGGAAATGGGCGGGGAGGGCCTTCGT | |
| | GCGTCGCCGCGCCGCCGTCCCCTTCTCCCTCTCCAGCCTCGGGGCTG | |
| | TCCGCGGGGGGACGGCTGCCTTCGGGGGGGACGGGGCAGGGCGGG | |
| | GTTCGGCTTCTGGCGTGTGACCGGCGGCTCTAGAGCCTCTGCTAACCA | |
| | TGTTCATGCCTTCTTCTTTTTCCTACAGCTCCTGGGCAACGTGCTGGTT | |
| | ATTGTGCTGTCTCATCATTTTGGCAAAGAATTGGAGAATAAACTAGTATT | |
| | CTTCTGGTCCCCACAGACTCAGAGAGAACCCGCCACCATGTTCGTGTT | |
| | CCTGGTGCTGCTGCCCCTGGTGAGCAGCCAGTGCGTGAACCTGACCA | |
| | CCAGGACCCAGCTGCCCCCCGCCTACACCAACAGCTTCACCAGGGGC | |
| | GTGTACTACCCCGACAAGGTGTTCAGGAGCAGCGTGCTGCACAGCACC | |
| | CAGGACCTGTTCCTGCCCTTCTTCAGCAACGTGACCTGGTTCCACGCC | |
| | ATCCACGTGAGCGGCACCAACGGCACCAAGAGGTTCGACAACCCCGT | |
| | GCTGCCCTTCAACGACGGCGTGTACTTCGCCAGCACCGAGAAGAGCAA | |
| | CATCATCAGGGGCTGGATCTTCGGCACCACCCTGGACAGCAAGACCCA | |
| | GAGCCTGCTGATCGTGAACAACGCCACCAACGTGGTGATCAAGGTGTG | |
| | CGAGTTCCAGTTCTGCAACGACCCCTTCCTGGGCGTGTACTACCACAA | |
| | GAACAACAAGAGCTGGATGGAGAGCGAGTTCAGGGTGTACAGCAGCG | |
| | CCAACAACTGCACCTTCGAGTACGTGAGCCAGCCCTTCCTGATGGACC | |
| | TGGAGGGCAAGCAGGGCAACTTCAAGAACCTGAGGGAGTTCGTGTTCA | |
| | AGAACATCGACGGCTACTTCAAGATCTACAGCAAGCACACCCCCATCAA | |
| | CCTGGTGAGGGACCTGCCCCAGGGCTTCAGCGCCCTGGAGCCCCTGG | |
| | TGGACCTGCCCATCGGCATCAACATCACCAGGTTCCAGACCCTGCTGG | |
| | CCCTGCACAGGAGCTACCTGACCCCCGGCGACAGCAGCAGCGGCTGG | |
| | ACCGCCGGCGCCGCCGCCTACTACGTGGGCTACCTGCAGCCCAGGAC | |
| | CTTCCTGCTGAAGTACAACGAGAACGGCACCATCACCGACGCCGTGGA | |
| | CTGCGCCCTGGACCCCCTGAGCGAGACCAAGTGCACCCTGAAGAGCTT | |
| | CACCGTGGAGAAGGGCATCTACCAGACCAGCAACTTCAGGGTGCAGCC | |
| | CACCGAGAGCATCGTGAGGTTCCCCAACATCACCAACCTGTGCCCCTT | |
| | CGGCGAGGTGTTCAACGCCACCAGGTTCGCCAGCGTGTACGCCTGGA | |
| | ACAGGAAGAGGATCAGCAACTGCGTGGCCGACTACAGCGTGCTGTACA | |
| | ACAGCGCCAGCTTCAGCACCTTCAAGTGCTACGGCGTGAGCCCCACCA | |
| | AGCTGAACGACCTGTGCTTCACCAACGTGTACGCCGACAGCTTCGTGA | |
| | TCAGGGGCGACGAGGTGAGGCAGATCGCCCCCGGCCAGACCGGCAA | |
| | GATCGCCGACTACAACTACAAGCTGCCCGACGACTTCACCGGCTGCGT | |
| | GATCGCCTGGAACAGCAACAACCTGGACAGCAAGGTGGGCGGCAACT | |
| | ACAACTACCTGTACAGGCTGTTCAGGAAGAGCAACCTGAAGCCCTTCG | |
| | AGAGGGACATCAGCACCGAGATCTACCAGGCCGGCAGCACCCCCTGC | |
| | AACGGCGTGGAGGGCTTCAACTGCTACTTCCCCCTGCAGAGCTACGGC | |
| | TTCCAGCCCACCAACGGCGTGGGCTACCAGCCCTACAGGGTGGTGGT | |
| | GCTGAGCTTCGAGCTGCTGCACGCCCCCGCCACCGTGTGCGGCCCCA | |
| | AGAAGAGCACCAACCTGGTGAAGAACAAGTGCGTGAACTTCAACTTCAA | |
| | CGGCCTGACCGGCACCGGCGTGCTGACCGAGAGCAACAAGAAGTTCC | |
| | TGCCCTTCCAGCAGTTCGGCAGGGACATCGCCGACACCACCGACGCC | |
| | GTGAGGGACCCCCAGACCCTGGAGATCCTGGACATCACCCCCTGCAG | |
| | CTTCGGCGGCGTGAGCGTGATCACCCCCGGCACCAACACCAGCAACC | |
| | AGGTGGCCGTGCTGTACCAGGACGTGAACTGCACCGAGGTGCCCGTG | |
| | GCCATCCACGCCGACCAGCTGACCCCCACCTGGAGGGTGTACAGCAC | |
| | CGGCAGCAACGTGTTCCAGACCAGGGCCGGCTGCCTGATCGGCGCCG | |
| | AGCACGTGAACAACAGCTACGAGTGCGACATCCCCATCGGCGCCGGC | |
| | ATCTGCGCCAGCTACCAGACCCAGACCAACAGCCCCGGCAGCGCCAG | |
| | CAGCGTGGCCAGCCAGAGCATCATCGCCTACACCATGAGCCTGGGCG | |
| | CCGAGAACAGCGTGGCCTACAGCAACAACAGCATCGCCATCCCCACCA | |
| | ACTTCACCATCAGCGTGACCACCGAGATCCTGCCCGTGAGCATGACCA | |
| | AGACCAGCGTGGACTGCACCATGTACATCTGCGGCGACAGCACCGAGT | |
| | GCAGCAACCTGCTGCTGCAGTACGGCAGCTTCTGCACCCAGCTGAACA | |
| | GGGCCCTGACCGGCATCGCCGTGGAGCAGGACAAGAACACCCAGGAG | |
| | GTGTTCGCCCAGGTGAAGCAGATCTACAAGACCCCCCCCATCAAGGAC | |
| | TTCGGCGGCTTCAACTTCAGCCAGATCCTGCCCGACCCCAGCAAGCCC | |
| | AGCAAGAGGAGCCCCATCGAGGACCTGCTGTTCAACAAGGTGACCCTG | |
| | GCCGACGCCGGCTTCATCAAGCAGTACGGCGACTGCCTGGGCGACAT | |
| | CGCCGCCAGGGACCTGATCTGCGCCCAGAAGTTCAACGGCCTGACCG | |
| | TGCTGCCCCCCCTGCTGACCGACGAGATGATCGCCCAGTACACCAGCG | |
| | CCCTGCTGGCCGGCACCATCACCAGCGGCTGGACCTTCGGCGCCGGC | |
| | CCCGCCCTGCAGATCCCCTTCCCCATGCAGATGGCCTACAGGTTCAAC | |
| | GGCATCGGCGTGACCCAGAACGTGCTGTACGAGAACCAGAAGCTGATC | |
| | GCCAACCAGTTCAACAGCGCCATCGGCAAGATCCAGGACAGCCTGAGC | |
| | AGCACCCCCAGCGCCCTGGGCAAGCTGCAGGACGTGGTGAACCAGAA | |
| | CGCCCAGGCCCTGAACACCCTGGTGAAGCAGCTGAGCAGCAACTTCG | |
| | GCGCCATCAGCAGCGTGCTGAACGACATCCTGAGCAGGCTGGACCCC | |
| | CCCGAGGCCGAGGTGCAGATCGACAGGCTGATCACCGGCAGGCTGCA | |
| | GAGCCTGCAGACCTACGTGACCCAGCAGCTGATCAGGGCCGCCGAGA | |
| | TCAGGGCCAGCGCCAACCTGGCCGCCACCAAGATGAGCGAGTGCGTG | |
| | CTGGGCCAGAGCAAGAGGGTGGACTTCTGCGGCAAGGGCTACCACCT | |
| | GATGAGCTTCCCCCAGAGCGCCCCCCACGGCGTGGTGTTCCTGCACG | |
| | TGACCTACGTGCCCGCCCAGGAGAAGAACTTCACCACCGCCCCCGCCA | |
| | TCTGCCACGACGGCAAGGCCCACTTCCCCAGGGAGGGCGTGTTCGTG | |
| | AGCAACGGCACCCACTGGTTCGTGACCCAGAGGAACTTCTACGAGCCC | |
| | CAGATCATCACCACCGACAACACCTTCGTGAGCGGCAACTGCGACGTG | |
| | GTGATCGGCATCGTGAACAACACCGTGTACGACCCCCTGCAGCCCGAG | |
| | CTGGACAGCTTCAAGGAGGAGCTGGACAAGTACTTCAAGAACCACACC | |
| | AGCCCCGACGTGGACCTGGGCGACATCAGCGGCATCAACGCCAGCGT | |
| | GGTGAACATCCAGAAGGAGATCGACAGGCTGAACGAGGTGGCCAAGA | |
| | ACCTGAACGAGAGCCTGATCGACCTGCAGGAGCTGGGCAAGTACGAG | |
| | CAGTACATCAAGTGGCCCTGGTACATCTGGCTGGGCTTCATCGCCGGC | |
| | CTGATCGCCATCGTGATGGTGACCATCATGCTGTGCTGCATGACCAGC | |
| | TGCTGCAGCTGCCTGAAGGGCTGCTGCAGCTGCGGCAGCTGCTGCAA | |
| | GTTCGACGAGGACGACAGCGAGCCCGTGCTGAAGGGCGTGAAGCTGC | |
| | ACTACACCGGAAGCGGAGCCACGAACTTCTCTCTGTTAAAGCAAGCAG | |
| | 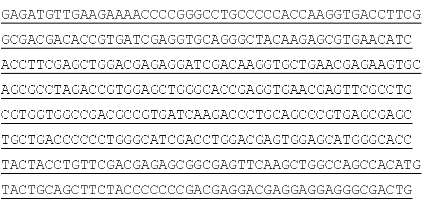 | |
| | 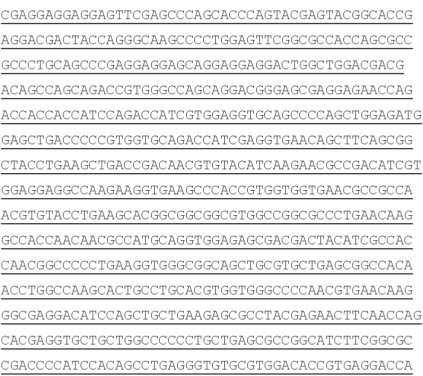 | |
| | 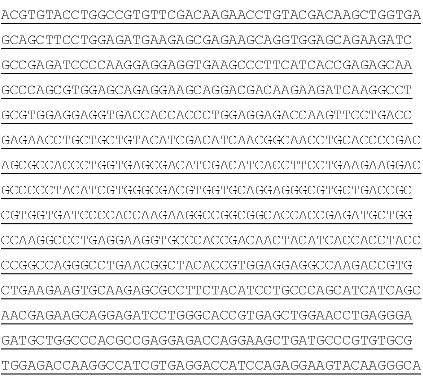 | |
| | 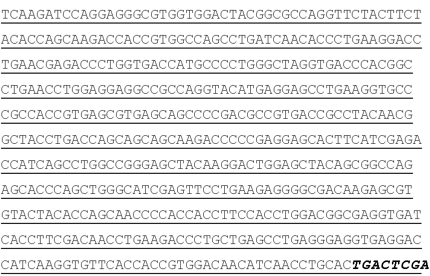 | |
| | 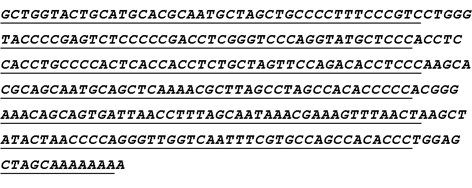 | |
| 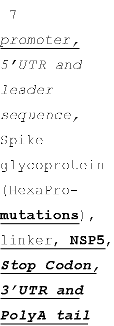 | CTCGACATTGATTATTGACTAGTTATTAATAGTAATCAATTACGGGGTCA TTAGTTCATAGCCCATATATGGAGTTCCGCGTTACATAACTTACGGTAAA TGGCCCGCCTGGCTGACCGCCCAACGACCCCCGCCCATTGACGTCAAT AATGACGTATGTTCCCATAGTAACGCCAATAGGGACTTTCCATTGACGT CAATGGGTGGAGTATTTACGGTAAACTGCCCACTTGGCAGTACATCAAG TGTATCATATGCCAAGTACGCCCCCTATTGACGTCAATGACGGTAAATG GCCCGCCTGGCATTATGCCCAGTACATGACCTTATGGGACTTTCCTACT TGGCAGTACATCTACGTATTAGTCATCGCTATTACCATGGTCGAGGTGA GCCCCACGTTCTGCTTCACTCTCCCCATCTCCCCCCCCTCCCCACCCC CAATTTTGTATTTATTTATTTTTTAATTATTTTGTGCAGCGATGGGGGCG GGGGGGGGGGGGGGGCGCGCGCCAGGCGGGGCGGGGCGGGGCGA GGGGCGGGGCGGGGCGAGGCGGAGAGGTGCGGCGGCAGCCAATCA GAGCGGCGCGCTCCGAAAGTTTCCTTTTATGGCGAGGCGGCGGCGGC GGCGGCCCTATAAAAAGCGAAGCGCGCGGCGGGCGGGAGTCGCTGC GCGCTGCCTTCGCCCCGTGCCCCGCTCCGCCGCCGCCTCGCGCCGCC CGCCCCGGCTCTGACTGACCGCGTTACTCCCACAGGTGAGCGGGCGG GACGGCCCTTCTCCTCCGGGCTGTAATTAGCGCTTGGTTTAATGACGG CTTGTTTCTTTTCTGTGGCTGCGTGAAAGCCTTGAGGGGCTCCGGGAG GGCCCTTTGTGCGGGGGGAGCGGCTCGGGGGGTGCGTGCGTGTGTG TGTGCGTGGGGAGCGCCGCGTGCGGCTCCGCGCTGCCCGGCGGCTG TGAGCGCTGCGGGCGCGGCGCGGGGCTTTGTGCGCTCCGCAGTGTGC | 145 |
| | GCGAGGGGAGCGCGGCCGGGGGCGGTGCCCCGCGGTGCGGGGGGG | |
| | GCTGCGAGGGGAACAAAGGCTGCGTGCGGGGTGTGTGCGTGGGGGG | |
| | GTGAGCAGGGGGTGTGGGCGCGTCGGTCGGGCTGCAACCCCCCCTG | |
| | CACCCCCCTCCCCGAGTTGCTGAGCACGGCCCGGCTTCGGGTGCGGG | |
| | GCTCCGTACGGGGCGTGGCGCGGGGCTCGCCGTGCCGGGCGGGGGG | |
| | TGGCGGCAGGTGGGGGTGCCGGGCGGGGCGGGGCCGCCTCGGGCC | |
| | GGGGAGGGCTCGGGGGAGGGGCGCGGCGGCCCCCGGAGCGCCGGC | |
| | GGCTGTCGAGGCGCGGCGAGCCGCAGCCATTGCCTTTTATGGTAATCG | |
| | TGCGAGAGGGCGCAGGGACTTCCTTTGTCCCAAATCTGTGCGGAGCCG | |
| | AAATCTGGGAGGCGCCGCCGCACCCCCTCTAGCGGGCGCGGGGCGAA | |
| | GCGGTGCGGCGCCGGCAGGAAGGAAATGGGCGGGGAGGGCCTTCGT | |
| | GCGTCGCCGCGCCGCCGTCCCCTTCTCCCTCTCCAGCCTCGGGGCTG | |
| | TCCGCGGGGGGACGGCTGCCTTCGGGGGGGACGGGGCAGGGCGGG | |
| | GTTCGGCTTCTGGCGTGTGACCGGCGGCTCTAGAGCCTCTGCTAACCA | |
| | TGTTCATGCCTTCTTCTTTTTCCTACAGCTCCTGGGCAACGTGCTGGTT | |
| | ATTGTGCTGTCTCATCATTTTGGCAAAGAATTGGAGAATAAACTAGTATT | |
| | CTTCTGGTCCCCACAGACTCAGAGAGAACCCGCCACCATGTTCGTGTT | |
| | CCTGGTGCTGCTGCCCCTGGTGAGCAGCCAGTGCGTGAACCTGACCA | |
| | CCAGGACCCAGCTGCCCCCCGCCTACACCAACAGCTTCACCAGGGGC | |
| | GTGTACTACCCCGACAAGGTGTTCAGGAGCAGCGTGCTGCACAGCACC | |
| | CAGGACCTGTTCCTGCCCTTCTTCAGCAACGTGACCTGGTTCCACGCC | |
| | ATCCACGTGAGCGGCACCAACGGCACCAAGAGGTTCGACAACCCCGT | |
| | GCTGCCCTTCAACGACGGCGTGTACTTCGCCAGCACCGAGAAGAGCAA | |
| | CATCATCAGGGGCTGGATCTTCGGCACCACCCTGGACAGCAAGACCCA | |
| | GAGCCTGCTGATCGTGAACAACGCCACCAACGTGGTGATCAAGGTGTG | |
| | CGAGTTCCAGTTCTGCAACGACCCCTTCCTGGGCGTGTACTACCACAA | |
| | GAACAACAAGAGCTGGATGGAGAGCGAGTTCAGGGTGTACAGCAGCG | |
| | CCAACAACTGCACCTTCGAGTACGTGAGCCAGCCCTTCCTGATGGACC | |
| | TGGAGGGCAAGCAGGGCAACTTCAAGAACCTGAGGGAGTTCGTGTTCA | |
| | AGAACATCGACGGCTACTTCAAGATCTACAGCAAGCACACCCCCATCAA | |
| | CCTGGTGAGGGACCTGCCCCAGGGCTTCAGCGCCCTGGAGCCCCTGG | |
| | TGGACCTGCCCATCGGCATCAACATCACCAGGTTCCAGACCCTGCTGG | |
| | CCCTGCACAGGAGCTACCTGACCCCCGGCGACAGCAGCAGCGGCTGG | |
| | ACCGCCGGCGCCGCCGCCTACTACGTGGGCTACCTGCAGCCCAGGAC | |
| | CTTCCTGCTGAAGTACAACGAGAACGGCACCATCACCGACGCCGTGGA | |
| | CTGCGCCCTGGACCCCCTGAGCGAGACCAAGTGCACCCTGAAGAGCTT | |
| | CACCGTGGAGAAGGGCATCTACCAGACCAGCAACTTCAGGGTGCAGCC | |
| | CACCGAGAGCATCGTGAGGTTCCCCAACATCACCAACCTGTGCCCCTT | |
| | CGGCGAGGTGTTCAACGCCACCAGGTTCGCCAGCGTGTACGCCTGGA | |
| | ACAGGAAGAGGATCAGCAACTGCGTGGCCGACTACAGCGTGCTGTACA | |
| | ACAGCGCCAGCTTCAGCACCTTCAAGTGCTACGGCGTGAGCCCCACCA | |
| | AGCTGAACGACCTGTGCTTCACCAACGTGTACGCCGACAGCTTCGTGA | |
| | TCAGGGGCGACGAGGTGAGGCAGATCGCCCCCGGCCAGACCGGCAA | |
| | GATCGCCGACTACAACTACAAGCTGCCCGACGACTTCACCGGCTGCGT | |
| | GATCGCCTGGAACAGCAACAACCTGGACAGCAAGGTGGGCGGCAACT | |
| | ACAACTACCTGTACAGGCTGTTCAGGAAGAGCAACCTGAAGCCCTTCG | |
| | AGAGGGACATCAGCACCGAGATCTACCAGGCCGGCAGCACCCCCTGC | |
| | AACGGCGTGGAGGGCTTCAACTGCTACTTCCCCCTGCAGAGCTACGGC | |
| | TTCCAGCCCACCAACGGCGTGGGCTACCAGCCCTACAGGGTGGTGGT | |
| | GCTGAGCTTCGAGCTGCTGCACGCCCCCGCCACCGTGTGCGGCCCCA | |
| | AGAAGAGCACCAACCTGGTGAAGAACAAGTGCGTGAACTTCAACTTCAA | |
| | CGGCCTGACCGGCACCGGCGTGCTGACCGAGAGCAACAAGAAGTTCC | |
| | TGCCCTTCCAGCAGTTCGGCAGGGACATCGCCGACACCACCGACGCC | |
| | GTGAGGGACCCCCAGACCCTGGAGATCCTGGACATCACCCCCTGCAG | |
| | CTTCGGCGGCGTGAGCGTGATCACCCCCGGCACCAACACCAGCAACC | |
| | AGGTGGCCGTGCTGTACCAGGACGTGAACTGCACCGAGGTGCCCGTG | |
| | GCCATCCACGCCGACCAGCTGACCCCCACCTGGAGGGTGTACAGCAC | |
| | CGGCAGCAACGTGTTCCAGACCAGGGCCGGCTGCCTGATCGGCGCCG | |
| | AGCACGTGAACAACAGCTACGAGTGCGACATCCCCATCGGCGCCGGC | |
| | ATCTGCGCCAGCTACCAGACCCAGACCAACAGCCCCGGCAGCGCCAG | |
| | CAGCGTGGCCAGCCAGAGCATCATCGCCTACACCATGAGCCTGGGCG | |
| | CCGAGAACAGCGTGGCCTACAGCAACAACAGCATCGCCATCCCCACCA | |
| | ACTTCACCATCAGCGTGACCACCGAGATCCTGCCCGTGAGCATGACCA | |
| | AGACCAGCGTGGACTGCACCATGTACATCTGCGGCGACAGCACCGAGT | |
| | GCAGCAACCTGCTGCTGCAGTACGGCAGCTTCTGCACCCAGCTGAACA | |
| | GGGCCCTGACCGGCATCGCCGTGGAGCAGGACAAGAACACCCAGGAG | |
| | GTGTTCGCCCAGGTGAAGCAGATCTACAAGACCCCCCCCATCAAGGAC | |
| | TTCGGCGGCTTCAACTTCAGCCAGATCCTGCCCGACCCCAGCAAGCCC | |
| | AGCAAGAGGAGCCCCATCGAGGACCTGCTGTTCAACAAGGTGACCCTG | |
| | GCCGACGCCGGCTTCATCAAGCAGTACGGCGACTGCCTGGGCGACAT | |
| | CGCCGCCAGGGACCTGATCTGCGCCCAGAAGTTCAACGGCCTGACCG | |
| | TGCTGCCCCCCCTGCTGACCGACGAGATGATCGCCCAGTACACCAGCG | |
| | CCCTGCTGGCCGGCACCATCACCAGCGGCTGGACCTTCGGCGCCGGC | |
| | CCCGCCCTGCAGATCCCCTTCCCCATGCAGATGGCCTACAGGTTCAAC | |
| | GGCATCGGCGTGACCCAGAACGTGCTGTACGAGAACCAGAAGCTGATC | |
| | GCCAACCAGTTCAACAGCGCCATCGGCAAGATCCAGGACAGCCTGAGC | |
| | AGCACCCCCAGCGCCCTGGGCAAGCTGCAGGACGTGGTGAACCAGAA | |
| | CGCCCAGGCCCTGAACACCCTGGTGAAGCAGCTGAGCAGCAACTTCG | |
| | GCGCCATCAGCAGCGTGCTGAACGACATCCTGAGCAGGCTGGACCCC | |
| | CCCGAGGCCGAGGTGCAGATCGACAGGCTGATCACCGGCAGGCTGCA | |
| | GAGCCTGCAGACCTACGTGACCCAGCAGCTGATCAGGGCCGCCGAGA | |
| | TCAGGGCCAGCGCCAACCTGGCCGCCACCAAGATGAGCGAGTGCGTG | |
| | CTGGGCCAGAGCAAGAGGGTGGACTTCTGCGGCAAGGGCTACCACCT | |
| | GATGAGCTTCCCCCAGAGCGCCCCCCACGGCGTGGTGTTCCTGCACG | |
| | TGACCTACGTGCCCGCCCAGGAGAAGAACTTCACCACCGCCCCCGCCA | |
| | TCTGCCACGACGGCAAGGCCCACTTCCCCAGGGAGGGCGTGTTCGTG | |
| | AGCAACGGCACCCACTGGTTCGTGACCCAGAGGAACTTCTACGAGCCC | |
| | CAGATCATCACCACCGACAACACCTTCGTGAGCGGCAACTGCGACGTG | |
| | GTGATCGGCATCGTGAACAACACCGTGTACGACCCCCTGCAGCCCGAG | |
| | CTGGACAGCTTCAAGGAGGAGCTGGACAAGTACTTCAAGAACCACACC | |
| | AGCCCCGACGTGGACCTGGGCGACATCAGCGGCATCAACGCCAGCGT | |
| | GGTGAACATCCAGAAGGAGATCGACAGGCTGAACGAGGTGGCCAAGA | |
| | ACCTGAACGAGAGCCTGATCGACCTGCAGGAGCTGGGCAAGTACGAG | |
| | CAGTACATCAAGTGGCCCTGGTACATCTGGCTGGGCTTCATCGCCGGC | |
| | CTGATCGCCATCGTGATGGTGACCATCATGCTGTGCTGCATGACCAGC | |
| | TGCTGCAGCTGCCTGAAGGGCTGCTGCAGCTGCGGCAGCTGCTGCAA | |
| | GTTCGACGAGGACGACAGCGAGCCCGTGCTGAAGGGCGTGAAGCTGC | |
| | ACTACACCGGAAGCGGAGCCACGAACTTCTCTCTGTTAAAGCAAGCAG | |
| | 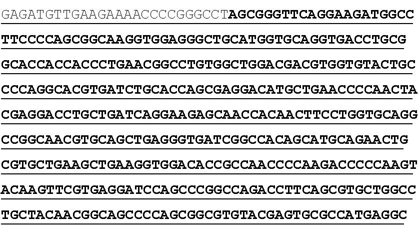 | |
| | 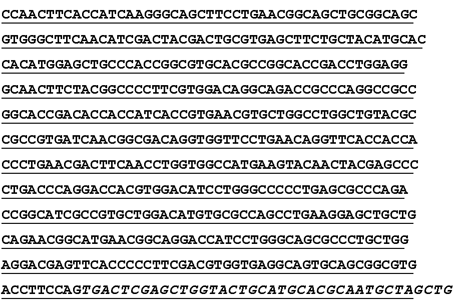 | |
| | 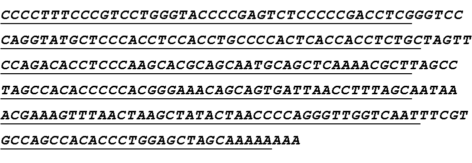 | |
| 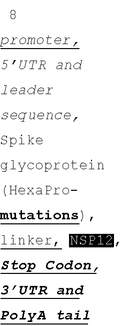 | CTCGACATTGATTATTGACTAGTTATTAATAGTAATCAATTACGGGGTCA TTAGTTCATAGCCCATATATGGAGTTCCGCGTTACATAACTTACGGTAAA TGGCCCGCCTGGCTGACCGCCCAACGACCCCCGCCCATTGACGTCAAT AATGACGTATGTTCCCATAGTAACGCCAATAGGGACTTTCCATTGACGT CAATGGGTGGAGTATTTACGGTAAACTGCCCACTTGGCAGTACATCAAG TGTATCATATGCCAAGTACGCCCCCTATTGACGTCAATGACGGTAAATG GCCCGCCTGGCATTATGCCCAGTACATGACCTTATGGGACTTTCCTACT TGGCAGTACATCTACGTATTAGTCATCGCTATTACCATGGTCGAGGTGA GCCCCACGTTCTGCTTCACTCTCCCCATCTCCCCCCCCTCCCCACCCC CAATTTTGTATTTATTTATTTTTTAATTATTTTGTGCAGCGATGGGGGCG GGGGGGGGGGGGGGGCGCGCGCCAGGCGGGGCGGGGCGGGGCGA GGGGCGGGGCGGGGCGAGGCGGAGAGGTGCGGCGGCAGCCAATCA GAGCGGCGCGCTCCGAAAGATTCCTTTTATGGCGAGGCGGCGGCGGC GGCGGCCCTATAAAAAGCGAAGCGCGCGGCGGGCGGGAGTCGCTGC GCGCTGCCTTCGCCCCGTGCCCCGCTCCGCCGCCGCCTCGCGCCGCC CGCCCCGGCTCTGACTGACCGCGTTACTCCCACAGGTGAGCGGGCGG GACGGCCCTTCTCCTCCGGGCTGTAATTAGCGCTTGGTTTAATGACGG CTTGTTTCTTTTCTGTGGCTGCGTGAAAGCCTTGAGGGGCTCCGGGAG GGCCCTTTGTGCGGGGGGAGCGGCTCGGGGGGTGCGTGCGTGTGTG TGTGCGTGGGGAGCGCCGCGTGCGGCTCCGCGCTGCCCGGCGGCTG TGAGCGCTGCGGGCGCGGCGCGGGGCTTTGTGCGCTCCGCAGTGTGC | 146 |
| | GCGAGGGGAGCGCGGCCGGGGGCGGTGCCCCGCGGTGCGGGGGGG | |
| | GCTGCGAGGGGAACAAAGGCTGCGTGCGGGGTGTGTGCGTGGGGGG | |
| | GTGAGCAGGGGGTGTGGGCGCGTCGGTCGGGCTGCAACCCCCCCTG | |
| | CACCCCCCTCCCCGAGTTGCTGAGCACGGCCCGGCTTCGGGTGCGGG | |
| | GCTCCGTACGGGGCGTGGCGCGGGGCTCGCCGTGCCGGGCGGGGGG | |
| | TGGCGGCAGGTGGGGGTGCCGGGCGGGGCGGGGCCGCCTCGGGCC | |
| | GGGGAGGGCTCGGGGGAGGGGCGCGGCGGCCCCCGGAGCGCCGGC | |
| | GGCTGTCGAGGCGCGGCGAGCCGCAGCCATTGCCTTTTATGGTAATCG | |
| | TGCGAGAGGGCGCAGGGACTTCCTTTGTCCCAAATCTGTGCGGAGCCG | |
| | AAATCTGGGAGGCGCCGCCGCACCCCCTCTAGCGGGCGCGGGGCGAA | |
| | GCGGTGCGGCGCCGGCAGGAAGGAAATGGGCGGGGAGGGCCTTCGT | |
| | GCGTCGCCGCGCCGCCGTCCCCTTCTCCCTCTCCAGCCTCGGGGCTG | |
| | TCCGCGGGGGGACGGCTGCCTTCGGGGGGGACGGGGCAGGGCGGG | |
| | GTTCGGCTTCTGGCGTGTGACCGGCGGCTCTAGAGCCTCTGCTAACCA | |
| | TGTTCATGCCTTCTTCTTTTTCCTACAGCTCCTGGGCAACGTGCTGGTT | |
| | ATTGTGCTGTCTCATCATTTTGGCAAAGAATTGGAGAATAAACTAGTATT | |
| | CTTCTGGTCCCCACAGACTCAGAGAGAACCCGCCACCATGTTCGTGTT | |
| | CCTGGTGCTGCTGCCCCTGGTGAGCAGCCAGTGCGTGAACCTGACCA | |
| | CCAGGACCCAGCTGCCCCCCGCCTACACCAACAGCTTCACCAGGGGC | |
| | GTGTACTACCCCGACAAGGTGTTCAGGAGCAGCGTGCTGCACAGCACC | |
| | CAGGACCTGTTCCTGCCCTTCTTCAGCAACGTGACCTGGTTCCACGCC | |
| | ATCCACGTGAGCGGCACCAACGGCACCAAGAGGTTCGACAACCCCGT | |
| | GCTGCCCTTCAACGACGGCGTGTACTTCGCCAGCACCGAGAAGAGCAA | |
| | CATCATCAGGGGCTGGATCTTCGGCACCACCCTGGACAGCAAGACCCA | |
| | GAGCCTGCTGATCGTGAACAACGCCACCAACGTGGTGATCAAGGTGTG | |
| | CGAGTTCCAGTTCTGCAACGACCCCTTCCTGGGCGTGTACTACCACAA | |
| | GAACAACAAGAGCTGGATGGAGAGCGAGTTCAGGGTGTACAGCAGCG | |
| | CCAACAACTGCACCTTCGAGTACGTGAGCCAGCCCTTCCTGATGGACC | |
| | TGGAGGGCAAGCAGGGCAACTTCAAGAACCTGAGGGAGTTCGTGTTCA | |
| | AGAACATCGACGGCTACTTCAAGATCTACAGCAAGCACACCCCCATCAA | |
| | CCTGGTGAGGGACCTGCCCCAGGGCTTCAGCGCCCTGGAGCCCCTGG | |
| | TGGACCTGCCCATCGGCATCAACATCACCAGGTTCCAGACCCTGCTGG | |
| | CCCTGCACAGGAGCTACCTGACCCCCGGCGACAGCAGCAGCGGCTGG | |
| | ACCGCCGGCGCCGCCGCCTACTACGTGGGCTACCTGCAGCCCAGGAC | |
| | CTTCCTGCTGAAGTACAACGAGAACGGCACCATCACCGACGCCGTGGA | |
| | CTGCGCCCTGGACCCCCTGAGCGAGACCAAGTGCACCCTGAAGAGCTT | |
| | CACCGTGGAGAAGGGCATCTACCAGACCAGCAACTTCAGGGTGCAGCC | |
| | CACCGAGAGCATCGTGAGGTTCCCCAACATCACCAACCTGTGCCCCTT | |
| | CGGCGAGGTGTTCAACGCCACCAGGTTCGCCAGCGTGTACGCCTGGA | |
| | ACAGGAAGAGGATCAGCAACTGCGTGGCCGACTACAGCGTGCTGTACA | |
| | ACAGCGCCAGCTTCAGCACCTTCAAGTGCTACGGCGTGAGCCCCACCA | |
| | AGCTGAACGACCTGTGCTTCACCAACGTGTACGCCGACAGCTTCGTGA | |
| | TCAGGGGCGACGAGGTGAGGCAGATCGCCCCCGGCCAGACCGGCAA | |
| | GATCGCCGACTACAACTACAAGCTGCCCGACGACTTCACCGGCTGCGT | |
| | GATCGCCTGGAACAGCAACAACCTGGACAGCAAGGTGGGCGGCAACT | |
| | ACAACTACCTGTACAGGCTGTTCAGGAAGAGCAACCTGAAGCCCTTCG | |
| | AGAGGGACATCAGCACCGAGATCTACCAGGCCGGCAGCACCCCCTGC | |
| | AACGGCGTGGAGGGCTTCAACTGCTACTTCCCCCTGCAGAGCTACGGC | |
| | TTCCAGCCCACCAACGGCGTGGGCTACCAGCCCTACAGGGTGGTGGT | |
| | GCTGAGCTTCGAGCTGCTGCACGCCCCCGCCACCGTGTGCGGCCCCA | |
| | AGAAGAGCACCAACCTGGTGAAGAACAAGTGCGTGAACTTCAACTTCAA | |
| | CGGCCTGACCGGCACCGGCGTGCTGACCGAGAGCAACAAGAAGTTCC | |
| | TGCCCTTCCAGCAGTTCGGCAGGGACATCGCCGACACCACCGACGCC | |
| | GTGAGGGACCCCCAGACCCTGGAGATCCTGGACATCACCCCCTGCAG | |
| | CTTCGGCGGCGTGAGCGTGATCACCCCCGGCACCAACACCAGCAACC | |
| | AGGTGGCCGTGCTGTACCAGGACGTGAACTGCACCGAGGTGCCCGTG | |
| | GCCATCCACGCCGACCAGCTGACCCCCACCTGGAGGGTGTACAGCAC | |
| | CGGCAGCAACGTGTTCCAGACCAGGGCCGGCTGCCTGATCGGCGCCG | |
| | AGCACGTGAACAACAGCTACGAGTGCGACATCCCCATCGGCGCCGGC | |
| | ATCTGCGCCAGCTACCAGACCCAGACCAACAGCCCCGGCAGCGCCAG | |
| | CAGCGTGGCCAGCCAGAGCATCATCGCCTACACCATGAGCCTGGGCG | |
| | CCGAGAACAGCGTGGCCTACAGCAACAACAGCATCGCCATCCCCACCA | |
| | ACTTCACCATCAGCGTGACCACCGAGATCCTGCCCGTGAGCATGACCA | |
| | AGACCAGCGTGGACTGCACCATGTACATCTGCGGCGACAGCACCGAGT | |
| | GCAGCAACCTGCTGCTGCAGTACGGCAGCTTCTGCACCCAGCTGAACA | |
| | GGGCCCTGACCGGCATCGCCGTGGAGCAGGACAAGAACACCCAGGAG | |
| | GTGTTCGCCCAGGTGAAGCAGATCTACAAGACCCCCCCCATCAAGGAC | |
| | TTCGGCGGCTTCAACTTCAGCCAGATCCTGCCCGACCCCAGCAAGCCC | |
| | AGCAAGAGGAGCCCCATCGAGGACCTGCTGTTCAACAAGGTGACCCTG | |
| | GCCGACGCCGGCTTCATCAAGCAGTACGGCGACTGCCTGGGCGACAT | |
| | CGCCGCCAGGGACCTGATCTGCGCCCAGAAGTTCAACGGCCTGACCG | |
| | TGCTGCCCCCCCTGCTGACCGACGAGATGATCGCCCAGTACACCAGCG | |
| | CCCTGCTGGCCGGCACCATCACCAGCGGCTGGACCTTCGGCGCCGGC | |
| | CCCGCCCTGCAGATCCCCTTCCCCATGCAGATGGCCTACAGGTTCAAC | |
| | GGCATCGGCGTGACCCAGAACGTGCTGTACGAGAACCAGAAGCTGATC | |
| | GCCAACCAGTTCAACAGCGCCATCGGCAAGATCCAGGACAGCCTGAGC | |
| | AGCACCCCCAGCGCCCTGGGCAAGCTGCAGGACGTGGTGAACCAGAA | |
| | CGCCCAGGCCCTGAACACCCTGGTGAAGCAGCTGAGCAGCAACTTCG | |
| | GCGCCATCAGCAGCGTGCTGAACGACATCCTGAGCAGGCTGGACCCC | |
| | CCCGAGGCCGAGGTGCAGATCGACAGGCTGATCACCGGCAGGCTGCA | |
| | GAGCCTGCAGACCTACGTGACCCAGCAGCTGATCAGGGCCGCCGAGA | |
| | TCAGGGCCAGCGCCAACCTGGCCGCCACCAAGATGAGCGAGTGCGTG | |
| | CTGGGCCAGAGCAAGAGGGTGGACTTCTGCGGCAAGGGCTACCACCT | |
| | GATGAGCTTCCCCCAGAGCGCCCCCCACGGCGTGGTGTTCCTGCACG | |
| | TGACCTACGTGCCCGCCCAGGAGAAGAACTTCACCACCGCCCCCGCCA | |
| | TCTGCCACGACGGCAAGGCCCACTTCCCCAGGGAGGGCGTGTTCGTG | |
| | AGCAACGGCACCCACTGGTTCGTGACCCAGAGGAACTTCTACGAGCCC | |
| | CAGATCATCACCACCGACAACACCTTCGTGAGCGGCAACTGCGACGTG | |
| | GTGATCGGCATCGTGAACAACACCGTGTACGACCCCCTGCAGCCCGAG | |
| | CTGGACAGCTTCAAGGAGGAGCTGGACAAGTACTTCAAGAACCACACC | |
| | AGCCCCGACGTGGACCTGGGCGACATCAGCGGCATCAACGCCAGCGT | |
| | GGTGAACATCCAGAAGGAGATCGACAGGCTGAACGAGGTGGCCAAGA | |
| | ACCTGAACGAGAGCCTGATCGACCTGCAGGAGCTGGGCAAGTACGAG | |
| | CAGTACATCAAGTGGCCCTGGTACATCTGGCTGGGCTTCATCGCCGGC | |
| | CTGATCGCCATCGTGATGGTGACCATCATGCTGTGCTGCATGACCAGC | |
| | TGCTGCAGCTGCCTGAAGGGCTGCTGCAGCTGCGGCAGCTGCTGCAA | |
| | GTTCGACGAGGACGACAGCGAGCCCGTGCTGAAGGGCGTGAAGCTGC | |
| | ACTACACCGGAAGCGGAGCCACGAACTTCTCTCTGTTAAAGCAAGCAG | |
| | 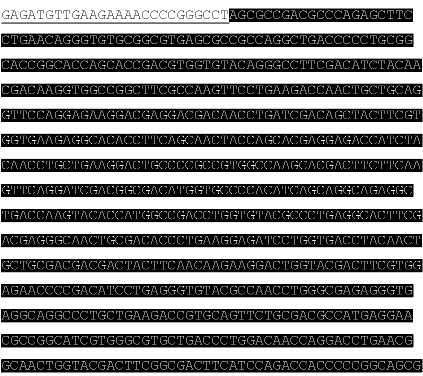 | |
| | 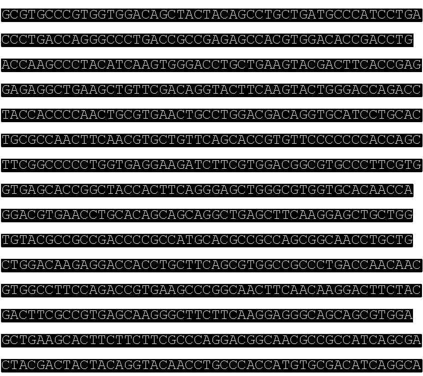 | |
| | 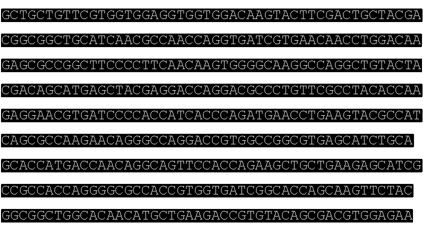 | |
| | 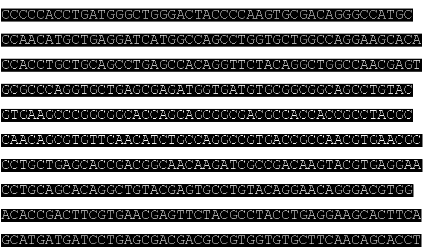 | |
| | 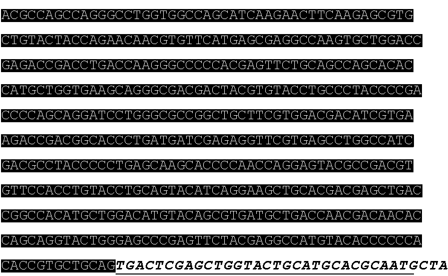 | |
| | 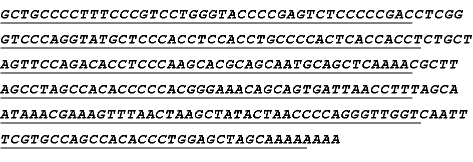 | |
| 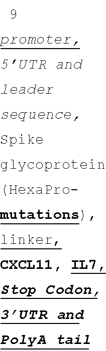 | CTCGACATTGATTATTGACTAGTTATTAATAGTAATCAATTACGGGGTCA TTAGTTCATAGCCCATATATGGAGTTCCGCGTTACATAACTTACGGTAAA TGGCCCGCCTGGCTGACCGCCCAACGACCCCCGCCCATTGACGTCAAT AATGACGTATGTTCCCATAGTAACGCCAATAGGGACTTTCCATTGACGT CAATGGGTGGAGTATTTACGGTAAACTGCCCACTTGGCAGTACATCAAG TGTATCATATGCCAAGTACGCCCCCTATTGACGTCAATGACGGTAAATG GCCCGCCTGGCATTATGCCCAGTACATGACCTTATGGGACTTTCCTACT TGGCAGTACATCTACGTATTAGTCATCGCTATTACCATGGTCGAGGTGA GCCCCACGTTCTGCTTCACTCTCCCCATCTCCCCCCCCTCCCCACCCC CAATTTTGTATTTATTTATTTTTTAATTATTTTGTGCAGCGATGGGGGCG GGGGGGGGGGGGGGGCGCGCGCCAGGCGGGGCGGGGCGGGGCGA GGGGCGGGGCGGGGCGAGGCGGAGAGGTGCGGCGGCAGCCAATCA GAGCGGCGCGCTCCGAAAGTTTCCTTTTATGGCGAGGCGGCGGCGGC GGCGGCCCTATAAAAAGCGAAGCGCGCGGCGGGCGGGAGTCGCTGC GCGCTGCCTTCGCCCCGTGCCCCGCTCCGCCGCCGCCTCGCGCCGCC CGCCCCGGCTCTGACTGACCGCGTTACTCCCACAGGTGAGCGGGCGG GACGGCCCTTCTCCTCCGGGCTGTAATTAGCGCTTGGTTTAATGACGG CTTGTTTCTTTTCTGTGGCTGCGTGAAAGCCTTGAGGGGCTCCGGGAG GGCCCTTTGTGCGGGGGGAGCGGCTCGGGGGGTGCGTGCGTGTGTG TGTGCGTGGGGAGCGCCGCGTGCGGCTCCGCGCTGCCCGGCGGCTG TGAGCGCTGCGGGCGCGGCGCGGGGCTTTGTGCGCTCCGCAGTGTGC GCGAGGGGAGCGCGGCCGGGGGCGGTGCCCCGCGGTGCGGGGGGG GCTGCGAGGGGAACAAAGGCTGCGTGCGGGGTGTGTGCGTGGGGGG | 147 |
| | GTGAGCAGGGGGTGTGGGCGCGTCGGTCGGGCTGCAACCCCCCCTG | |
| | CACCCCCCTCCCCGAGTTGCTGAGCACGGCCCGGCTTCGGGTGCGGG | |
| | GCTCCGTACGGGGCGTGGCGCGGGGCTCGCCGTGCCGGGCGGGGGG | |
| | TGGCGGCAGGTGGGGGTGCCGGGCGGGGCGGGGCCGCCTCGGGCC | |
| | GGGGAGGGCTCGGGGGAGGGGCGCGGCGGCCCCCGGAGCGCCGGC | |
| | GGCTGTCGAGGCGCGGCGAGCCGCAGCCATTGCCTTTTATGGTAATCG | |
| | TGCGAGAGGGCGCAGGGACTTCCTTTGTCCCAAATCTGTGCGGAGCCG | |
| | AAATCTGGGAGGCGCCGCCGCACCCCCTCTAGCGGGCGCGGGGCGAA | |
| | GCGGTGCGGCGCCGGCAGGAAGGAAATGGGCGGGGAGGGCCTTCGT | |
| | GCGTCGCCGCGCCGCCGTCCCCTTCTCCCTCTCCAGCCTCGGGGCTG | |
| | TCCGCGGGGGGACGGCTGCCTTCGGGGGGGACGGGGCAGGGCGGG | |
| | GTTCGGCTTCTGGCGTGTGACCGGCGGCTCTAGAGCCTCTGCTAACCA | |
| | TGTTCATGCCTTCTTCTTTTTCCTACAGCTCCTGGGCAACGTGCTGGTT | |
| | ATTGTGCTGTCTCATCATTTTGGCAAAGAATTGGAGAATAAACTAGTATT | |
| | CTTCTGGTCCCCACAGACTCAGAGAGAACCCGCCACCATGTTCGTGTT | |
| | CCTGGTGCTGCTGCCCCTGGTGAGCAGCCAGTGCGTGAACCTGACCA | |
| | CCAGGACCCAGCTGCCCCCCGCCTACACCAACAGCTTCACCAGGGGC | |
| | GTGTACTACCCCGACAAGGTGTTCAGGAGCAGCGTGCTGCACAGCACC | |
| | CAGGACCTGTTCCTGCCCTTCTTCAGCAACGTGACCTGGTTCCACGCC | |
| | ATCCACGTGAGCGGCACCAACGGCACCAAGAGGTTCGACAACCCCGT | |
| | GCTGCCCTTCAACGACGGCGTGTACTTCGCCAGCACCGAGAAGAGCAA | |
| | CATCATCAGGGGCTGGATCTTCGGCACCACCCTGGACAGCAAGACCCA | |
| | GAGCCTGCTGATCGTGAACAACGCCACCAACGTGGTGATCAAGGTGTG | |
| | CGAGTTCCAGTTCTGCAACGACCCCTTCCTGGGCGTGTACTACCACAA | |
| | GAACAACAAGAGCTGGATGGAGAGCGAGTTCAGGGTGTACAGCAGCG | |
| | CCAACAACTGCACCTTCGAGTACGTGAGCCAGCCCTTCCTGATGGACC | |
| | TGGAGGGCAAGCAGGGCAACTTCAAGAACCTGAGGGAGTTCGTGTTCA | |
| | AGAACATCGACGGCTACTTCAAGATCTACAGCAAGCACACCCCCATCAA | |
| | CCTGGTGAGGGACCTGCCCCAGGGCTTCAGCGCCCTGGAGCCCCTGG | |
| | TGGACCTGCCCATCGGCATCAACATCACCAGGTTCCAGACCCTGCTGG | |
| | CCCTGCACAGGAGCTACCTGACCCCCGGCGACAGCAGCAGCGGCTGG | |
| | ACCGCCGGCGCCGCCGCCTACTACGTGGGCTACCTGCAGCCCAGGAC | |
| | CTTCCTGCTGAAGTACAACGAGAACGGCACCATCACCGACGCCGTGGA | |
| | CTGCGCCCTGGACCCCCTGAGCGAGACCAAGTGCACCCTGAAGAGCTT | |
| | CACCGTGGAGAAGGGCATCTACCAGACCAGCAACTTCAGGGTGCAGCC | |
| | CACCGAGAGCATCGTGAGGTTCCCCAACATCACCAACCTGTGCCCCTT | |
| | CGGCGAGGTGTTCAACGCCACCAGGTTCGCCAGCGTGTACGCCTGGA | |
| | ACAGGAAGAGGATCAGCAACTGCGTGGCCGACTACAGCGTGCTGTACA | |
| | ACAGCGCCAGCTTCAGCACCTTCAAGTGCTACGGCGTGAGCCCCACCA | |
| | AGCTGAACGACCTGTGCTTCACCAACGTGTACGCCGACAGCTTCGTGA | |
| | TCAGGGGCGACGAGGTGAGGCAGATCGCCCCCGGCCAGACCGGCAA | |
| | GATCGCCGACTACAACTACAAGCTGCCCGACGACTTCACCGGCTGCGT | |
| | GATCGCCTGGAACAGCAACAACCTGGACAGCAAGGTGGGCGGCAACT | |
| | ACAACTACCTGTACAGGCTGTTCAGGAAGAGCAACCTGAAGCCCTTCG | |
| | AGAGGGACATCAGCACCGAGATCTACCAGGCCGGCAGCACCCCCTGC | |
| | AACGGCGTGGAGGGCTTCAACTGCTACTTCCCCCTGCAGAGCTACGGC | |
| | TTCCAGCCCACCAACGGCGTGGGCTACCAGCCCTACAGGGTGGTGGT | |
| | GCTGAGCTTCGAGCTGCTGCACGCCCCCGCCACCGTGTGCGGCCCCA | |
| | AGAAGAGCACCAACCTGGTGAAGAACAAGTGCGTGAACTTCAACTTCAA | |
| | CGGCCTGACCGGCACCGGCGTGCTGACCGAGAGCAACAAGAAGTTCC | |
| | TGCCCTTCCAGCAGTTCGGCAGGGACATCGCCGACACCACCGACGCC | |
| | GTGAGGGACCCCCAGACCCTGGAGATCCTGGACATCACCCCCTGCAG | |
| | CTTCGGCGGCGTGAGCGTGATCACCCCCGGCACCAACACCAGCAACC | |
| | AGGTGGCCGTGCTGTACCAGGACGTGAACTGCACCGAGGTGCCCGTG | |
| | GCCATCCACGCCGACCAGCTGACCCCCACCTGGAGGGTGTACAGCAC | |
| | CGGCAGCAACGTGTTCCAGACCAGGGCCGGCTGCCTGATCGGCGCCG | |
| | AGCACGTGAACAACAGCTACGAGTGCGACATCCCCATCGGCGCCGGC | |
| | ATCTGCGCCAGCTACCAGACCCAGACCAACAGCCCCGGCAGCGCCAG | |
| | CAGCGTGGCCAGCCAGAGCATCATCGCCTACACCATGAGCCTGGGCG | |
| | CCGAGAACAGCGTGGCCTACAGCAACAACAGCATCGCCATCCCCACCA | |
| | ACTTCACCATCAGCGTGACCACCGAGATCCTGCCCGTGAGCATGACCA | |
| | AGACCAGCGTGGACTGCACCATGTACATCTGCGGCGACAGCACCGAGT | |
| | GCAGCAACCTGCTGCTGCAGTACGGCAGCTTCTGCACCCAGCTGAACA | |
| | GGGCCCTGACCGGCATCGCCGTGGAGCAGGACAAGAACACCCAGGAG | |
| | GTGTTCGCCCAGGTGAAGCAGATCTACAAGACCCCCCCCATCAAGGAC | |
| | TTCGGCGGCTTCAACTTCAGCCAGATCCTGCCCGACCCCAGCAAGCCC | |
| | AGCAAGAGGAGCCCCATCGAGGACCTGCTGTTCAACAAGGTGACCCTG | |
| | GCCGACGCCGGCTTCATCAAGCAGTACGGCGACTGCCTGGGCGACAT | |
| | CGCCGCCAGGGACCTGATCTGCGCCCAGAAGTTCAACGGCCTGACCG | |
| | TGCTGCCCCCCCTGCTGACCGACGAGATGATCGCCCAGTACACCAGCG | |
| | CCCTGCTGGCCGGCACCATCACCAGCGGCTGGACCTTCGGCGCCGGC | |
| | CCCGCCCTGCAGATCCCCTTCCCCATGCAGATGGCCTACAGGTTCAAC | |
| | GGCATCGGCGTGACCCAGAACGTGCTGTACGAGAACCAGAAGCTGATC | |
| | GCCAACCAGTTCAACAGCGCCATCGGCAAGATCCAGGACAGCCTGAGC | |
| | AGCACCCCCAGCGCCCTGGGCAAGCTGCAGGACGTGGTGAACCAGAA | |
| | CGCCCAGGCCCTGAACACCCTGGTGAAGCAGCTGAGCAGCAACTTCG | |
| | GCGCCATCAGCAGCGTGCTGAACGACATCCTGAGCAGGCTGGACCCC | |
| | CCCGAGGCCGAGGTGCAGATCGACAGGCTGATCACCGGCAGGCTGCA | |
| | GAGCCTGCAGACCTACGTGACCCAGCAGCTGATCAGGGCCGCCGAGA | |
| | TCAGGGCCAGCGCCAACCTGGCCGCCACCAAGATGAGCGAGTGCGTG | |
| | CTGGGCCAGAGCAAGAGGGTGGACTTCTGCGGCAAGGGCTACCACCT | |
| | GATGAGCTTCCCCCAGAGCGCCCCCCACGGCGTGGTGTTCCTGCACG | |
| | TGACCTACGTGCCCGCCCAGGAGAAGAACTTCACCACCGCCCCCGCCA | |
| | TCTGCCACGACGGCAAGGCCCACTTCCCCAGGGAGGGCGTGTTCGTG | |
| | AGCAACGGCACCCACTGGTTCGTGACCCAGAGGAACTTCTACGAGCCC | |
| | CAGATCATCACCACCGACAACACCTTCGTGAGCGGCAACTGCGACGTG | |
| | GTGATCGGCATCGTGAACAACACCGTGTACGACCCCCTGCAGCCCGAG | |
| | CTGGACAGCTTCAAGGAGGAGCTGGACAAGTACTTCAAGAACCACACC | |
| | AGCCCCGACGTGGACCTGGGCGACATCAGCGGCATCAACGCCAGCGT | |
| | GGTGAACATCCAGAAGGAGATCGACAGGCTGAACGAGGTGGCCAAGA | |
| | ACCTGAACGAGAGCCTGATCGACCTGCAGGAGCTGGGCAAGTACGAG | |
| | CAGTACATCAAGTGGCCCTGGTACATCTGGCTGGGCTTCATCGCCGGC | |
| | CTGATCGCCATCGTGATGGTGACCATCATGCTGTGCTGCATGACCAGC | |
| | TGCTGCAGCTGCCTGAAGGGCTGCTGCAGCTGCGGCAGCTGCTGCAA | |
| | GTTCGACGAGGACGACAGCGAGCCCGTGCTGAAGGGCGTGAAGCTGC | |
| | ACTACACCGGAAGCGGAGCCACGAACTTCTCTCTGTTAAAGCAAGCAG | |
| | GAGATGTTGAAGAAAACCCCGGGCCTATGAACAGGAAGGTGACCGCC | |
| | ATCGCCCTGGCCGCCATCATCTGGGCCACCGCCGCCCAGGGCTTCCT | |
| | GATGTTCAAGCAGGGCAGGTGCCTGTGCATCGGCCCCGGCATGAAGG | |
| | CCGTGAAGATGGCCGAGATCGAGAAGGCCAGCGTGATCTACCCCAG | |
| | CAACGGCTGCGACAAGGTGGAGGTGATCGTGACCATGAAGGCCCAC | |
| | AAGAGGCAGAGGTGCCTGGACCCCAGGAGCAAGCAGGCCAGGCTGA | |
| | TCATGCAGGCCATCGAGAAGAAGAACTTCCTGAGGAGGCAGAACATG | |
| | TGAGGAAGCGGAGCCACGAACTTCTCTCTGTTAAAGCAAGCAGGAGAT | |
| | GTTGAAGAAAACCCCGGGCCTATGTTCCACGTGAGCTTCAGGTACATC | |
| | TTCGGCATCCCCCCCCTGATCCTGGTGCTGCTGCCCGTGACCAGCAG | |
| | CGAGTGCCACATCAAGGACAAGGAGGGCAAGGCCTACGAGAGCGTG | |
| | CTGATGATCAGCATCGACGAGCTGGACAAGATGACCGGCACCGACAG | |
| | CAACTGCCCCAACAACGAGCCCAACTTCTTCAGGAAGCACGTGTGCG | |
| | ACGACACCAAGGAGGCCGCCTTCCTGAACAGGGCCGCCAGGAAGCT | |
| | GAAGCAGTTCCTGAAGATGAACATCAGCGAGGAGTTCAACGTGCACC | |
| | TGCTGACCGTGAGCCAGGGCACCCAGACCCTGGTGAACTGCACCAGC | |
| | AAGGAGGAGAAGAACGTGAAGGAGCAGAAGAAGAACGACGCCTGCT | |
| | TCCTGAAGAGGCTGCTGAGGGAGATCAAGACCTGCTGGAACAAGATC | |
| | 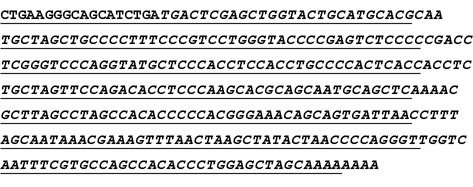 | |
| 10 | TAGTTATTAATAGTAATCAATTACGGGGTCATTAGTTCATAGCCCATATA | 295 |
| Ad5-Pan-CoV | TGGAGTTCCGCGTTACATAACTTACGGTAAATGGCCCGCCTGGCTGAC | |
| Spike-NSP1- | CGCCCAACGACCCCCGCCCATTGACGTCAATAATGACGTATGTTCCCAT | |
| NSP2 | AGTAACGCCAATAGGGACTTTCCATTGACGTCAATGGGTGGAGTATTTA | |
| | CGGTAAACTGCCCACTTGGCAGTACATCAAGTGTATCATATGCCAAGTA | |
| | CGCCCCCTATTGACGTCAATGACGGTAAATGGCCCGCCTGGCATTATG | |
| | CCCAGTACATGACCTTATGGGACTTTCCTACTTGGCAGTACATCTACGT | |
| | ATTAGTCATCGCTATTACCATGGTGATGCGGTTTTGGCAGTACATCAAT | |
| | GGGCGTGGATAGCGGTTTGACTCACGGGGATTTCCAAGTCTCCACCCC | |
| | ATTGACGTCAATGGGAGTTTGTTTTGGCACCAAAATCAACGGGACTTTC | |
| | CAAAATGTCGTAACAACTCCGCCCCATTGACGCAAATGGGCGGTAGGC | |
| | GTGTACGGTGGGAGGTCTATATAAGCAGAGCTGGTTTAGTGAACCGTC | |
| | AGATCGAGAATAAACTAGTATTCTTCTGGTCCCCACAGACTCAGAGAGA | |
| | ACCCGCCACCATGTTCGTGTTCCTGGTGCTGCTGCCCCTGGTGAGCAG | |
| | CCAGTGCGTGAACCTGACCACCAGGACCCAGCTGCCCCCCGCCTACA | |
| | CCAACAGCTTCACCAGGGGCGTGTACTACCCCGACAAGGTGTTCAGGA | |
| | GCAGCGTGCTGCACAGCACCCAGGACCTGTTCCTGCCCTTCTTCAGCA | |
| | ACGTGACCTGGTTCCACGCCATCCACGTGAGCGGCACCAACGGCACCA | |
| | AGAGGTTCGACAACCCCGTGCTGCCCTTCAACGACGGCGTGTACTTCG | |
| | CCAGCACCGAGAAGAGCAACATCATCAGGGGCTGGATCTTCGGCACCA | |
| | CCCTGGACAGCAAGACCCAGAGCCTGCTGATCGTGAACAACGCCACCA | |
| | ACGTGGTGATCAAGGTGTGCGAGTTCCAGTTCTGCAACGACCCCTTCC | |
| | TGGGCGTGTACTACCACAAGAACAACAAGAGCTGGATGGAGAGCGAGT | |
| | TCAGGGTGTACAGCAGCGCCAACAACTGCACCTTCGAGTACGTGAGCC | |
| | AGCCCTTCCTGATGGACCTGGAGGGCAAGCAGGGCAACTTCAAGAACC | |
| | TGAGGGAGTTCGTGTTCAAGAACATCGACGGCTACTTCAAGATCTACAG | |
| | CAAGCACACCCCCATCAACCTGGTGAGGGACCTGCCCCAGGGCTTCAG | |
| | CGCCCTGGAGCCCCTGGTGGACCTGCCCATCGGCATCAACATCACCAG | |
| | GTTCCAGACCCTGCTGGCCCTGCACAGGAGCTACCTGACCCCCGGCG | |
| | ACAGCAGCAGCGGCTGGACCGCCGGCGCCGCCGCCTACTACGTGGGC | |
| | TACCTGCAGCCCAGGACCTTCCTGCTGAAGTACAACGAGAACGGCACC | |
| | ATCACCGACGCCGTGGACTGCGCCCTGGACCCCCTGAGCGAGACCAA | |
| | GTGCACCCTGAAGAGCTTCACCGTGGAGAAGGGCATCTACCAGACCAG | |
| | CAACTTCAGGGTGCAGCCCACCGAGAGCATCGTGAGGTTCCCCAACAT | |
| | CACCAACCTGTGCCCCTTCGGCGAGGTGTTCAACGCCACCAGGTTCGC | |
| | CAGCGTGTACGCCTGGAACAGGAAGAGGATCAGCAACTGCGTGGCCG | |
| | ACTACAGCGTGCTGTACAACAGCGCCAGCTTCAGCACCTTCAAGTGCT | |
| | ACGGCGTGAGCCCCACCAAGCTGAACGACCTGTGCTTCACCAACGTGT | |
| | ACGCCGACAGCTTCGTGATCAGGGGCGACGAGGTGAGGCAGATCGCC | |
| | CCCGGCCAGACCGGCAAGATCGCCGACTACAACTACAAGCTGCCCGA | |
| | CGACTTCACCGGCTGCGTGATCGCCTGGAACAGCAACAACCTGGACAG | |
| | CAAGGTGGGCGGCAACTACAACTACCTGTACAGGCTGTTCAGGAAGAG | |
| | CAACCTGAAGCCCTTCGAGAGGGACATCAGCACCGAGATCTACCAGGC | |
| | CGGCAGCACCCCCTGCAACGGCGTGGAGGGCTTCAACTGCTACTTCCC | |
| | CCTGCAGAGCTACGGCTTCCAGCCCACCAACGGCGTGGGCTACCAGC | |
| | CCTACAGGGTGGTGGTGCTGAGCTTCGAGCTGCTGCACGCCCCCGCC | |
| | ACCGTGTGCGGCCCCAAGAAGAGCACCAACCTGGTGAAGAACAAGTGC | |
| | GTGAACTTCAACTTCAACGGCCTGACCGGCACCGGCGTGCTGACCGAG | |
| | AGCAACAAGAAGTTCCTGCCCTTCCAGCAGTTCGGCAGGGACATCGCC | |
| | GACACCACCGACGCCGTGAGGGACCCCCAGACCCTGGAGATCCTGGA | |
| | CATCACCCCCTGCAGCTTCGGCGGCGTGAGCGTGATCACCCCCGGCA | |
| | CCAACACCAGCAACCAGGTGGCCGTGCTGTACCAGGACGTGAACTGCA | |
| | CCGAGGTGCCCGTGGCCATCCACGCCGACCAGCTGACCCCCACCTGG | |
| | AGGGTGTACAGCACCGGCAGCAACGTGTTCCAGACCAGGGCCGGCTG | |
| | CCTGATCGGCGCCGAGCACGTGAACAACAGCTACGAGTGCGACATCCC | |
| | CATCGGCGCCGGCATCTGCGCCAGCTACCAGACCCAGACCAACAGCC | |
| | CCAGGAGGGCCAGGAGCGTGGCCAGCCAGAGCATCATCGCCTACACC | |
| | ATGAGCCTGGGCGCCGAGAACAGCGTGGCCTACAGCAACAACAGCAT | |
| | CGCCATCCCCACCAACTTCACCATCAGCGTGACCACCGAGATCCTGCC | |
| | CGTGAGCATGACCAAGACCAGCGTGGACTGCACCATGTACATCTGCGG | |
| | CGACAGCACCGAGTGCAGCAACCTGCTGCTGCAGTACGGCAGCTTCTG | |
| | CACCCAGCTGAACAGGGCCCTGACCGGCATCGCCGTGGAGCAGGACA | |
| | AGAACACCCAGGAGGTGTTCGCCCAGGTGAAGCAGATCTACAAGACCC | |
| | CCCCCATCAAGGACTTCGGCGGCTTCAACTTCAGCCAGATCCTGCCCG | |
| | ACCCCAGCAAGCCCAGCAAGAGGAGCTTCATCGAGGACCTGCTGTTCA | |
| | ACAAGGTGACCCTGGCCGACGCCGGCTTCATCAAGCAGTACGGCGACT | |
| | GCCTGGGCGACATCGCCGCCAGGGACCTGATCTGCGCCCAGAAGTTC | |
| | AACGGCCTGACCGTGCTGCCCCCCCTGCTGACCGACGAGATGATCGC | |
| | CCAGTACACCAGCGCCCTGCTGGCCGGCACCATCACCAGCGGCTGGA | |
| | CCTTCGGCGCCGGCGCCGCCCTGCAGATCCCCTTCGCCATGCAGATG | |
| | GCCTACAGGTTCAACGGCATCGGCGTGACCCAGAACGTGCTGTACGAG | |
| | AACCAGAAGCTGATCGCCAACCAGTTCAACAGCGCCATCGGCAAGATC | |
| | CAGGACAGCCTGAGCAGCACCGCCAGCGCCCTGGGCAAGCTGCAGGA | |
| | CGTGGTGAACCAGAACGCCCAGGCCCTGAACACCCTGGTGAAGCAGC | |
| | TGAGCAGCAACTTCGGCGCCATCAGCAGCGTGCTGAACGACATCCTGA | |
| | GCAGGCTGGACCCCCCCGAGGCCGAGGTGCAGATCGACAGGCTGATC | |
| | ACCGGCAGGCTGCAGAGCCTGCAGACCTACGTGACCCAGCAGCTGAT | |
| | CAGGGCCGCCGAGATCAGGGCCAGCGCCAACCTGGCCGCCACCAAGA | |
| | TGAGCGAGTGCGTGCTGGGCCAGAGCAAGAGGGTGGACTTCTGCGGC | |
| | AAGGGCTACCACCTGATGAGCTTCCCCCAGAGCGCCCCCCACGGCGT | |
| | GGTGTTCCTGCACGTGACCTACGTGCCCGCCCAGGAGAAGAACTTCAC | |
| | CACCGCCCCCGCCATCTGCCACGACGGCAAGGCCCACTTCCCCAGGG | |
| | AGGGCGTGTTCGTGAGCAACGGCACCCACTGGTTCGTGACCCAGAGG | |
| | AACTTCTACGAGCCCCAGATCATCACCACCGACAACACCTTCGTGAGC | |
| | GGCAACTGCGACGTGGTGATCGGCATCGTGAACAACACCGTGTACGAC | |
| | CCCCTGCAGCCCGAGCTGGACAGCTTCAAGGAGGAGCTGGACAAGTA | |
| | CTTCAAGAACCACACCAGCCCCGACGTGGACCTGGGCGACATCAGCG | |
| | GCATCAACGCCAGCGTGGTGAACATCCAGAAGGAGATCGACAGGCTGA | |
| | ACGAGGTGGCCAAGAACCTGAACGAGAGCCTGATCGACCTGCAGGAG | |
| | CTGGGCAAGTACGAGCAGTACATCAAGTGGCCCTGGTACATCTGGCTG | |
| | GGCTTCATCGCCGGCCTGATCGCCATCGTGATGGTGACCATCATGCTG | |
| | TGCTGCATGACCAGCTGCTGCAGCTGCCTGAAGGGCTGCTGCAGCTGC | |
| | GGCAGCTGCTGCAAGTTCGACGAGGACGACAGCGAGCCCGTGCTGAA | |
| | GGGCGTGAAGCTGCACTACACCGGAAGCGGAGCCACGAACTTCTCTCT | |
| | GTTAAAGCAAGCAGGAGATGTTGAAGAAAACCCCGGGCCTATGGAGAG | |
| | CCTGGTGCCCGGCTTCAACGAGAAGACCCACGTGCAGCTGAGCCTGC | |
| | CCGTGCTGCAGGTGAGGGACGTGCTGGTGAGGGGCTTCGGCGACAGC | |
| | GTGGAGGAGGTGCTGAGCGAGGCCAGGCAGCACCTGAAGGACGGCAC | |
| | CTGCGGCCTGGTGGAGGTGGAGAAGGGCGTGCTGCCCCAGCTGGAGC | |
| | AGCCCTACGTGTTCATCAAGAGGAGCGACGCCAGGACCGCCCCCCAC | |
| | GGCCACGTGATGGTGGAGCTGGTGGCCGAGCTGGAGGGCATCCAGTA | |
| | CGGCAGGAGCGGCGAGACCCTGGGCGTGCTGGTGCCCCACGTGGGC | |
| | GAGATCCCCGTGGCCTACAGGAAGGTGCTGCTGAGGAAGAACGGCAA | |
| | CAAGGGCGCCGGCGGCCACAGCTACGGCGCCGACCTGAAGAGCTTCG | |
| | ACCTGGGCGACGAGCTGGGCACCGACCCCTACGAGGACTTCCAGGAG | |
| | AACTGGAACACCAAGCACAGCAGCGGCGTGACCAGGGAGCTGATGAG | |
| | GGAGCTGAACGGCGGCGGAAGCGGAGAGGGCAGGGGAAGTCTTCTAA | |
| | CATGCGGGGACGTGGAGGAAAATCCCGGCCCCGCCTACACCAGGTAC | |
| | GTGGACAACAACTTCTGCGGCCCCGACGGCTACCCCCTGGAGTGCATC | |
| | AAGGACCTGCTGGCCAGGGCCGGCAAGGCCAGCTGCACCCTGAGCGA | |
| | GCAGCTGGACTTCATCGACACCAAGAGGGGCGTGTACTGCTGCAGGG | |
| | AGCACGAGCACGAGATCGCCTGGTACACCGAGAGGAGCGAGAAGAGC | |
| | TACGAGCTGCAGACCCCCTTCGAGATCAAGCTGGCCAAGAAGTTCGAC | |
| | ACCTTCAACGGCGAGTGCCCCAACTTCGTGTTCCCCCTGAACAGCATC | |
| | ATCAAGACCATCCAGCCCAGGGTGGAGAAGAAGAAGCTGGACGGCTTC | |
| | ATGGGCAGGATCAGGAGCGTGTACCCCGTGGCCAGCCCCAACGAGTG | |
| | CAACCAGATGTGCCTGAGCACCCTGATGAAGTGCGACCACTGCGGCGA | |
| | GACCAGCTGGCAGACCGGCGACTTCGTGAAGGCCACCTGCGAGTTCT | |
| | GCGGCACCGAGAACCTGACCAAGGAGGGCGCCACCACCTGCGGCTAC | |
| | CTGCCCCAGAACGCCGTGGTGAAGATCTACTGCCCCGCCTGCCACAAC | |
| | AGCGAGGTGGGCCCCGAGCACAGCCTGGCCGAGTACCACAACGAGAG | |
| | CGGCCTGAAGACCATCCTGAGGAAGGGCGGCAGGACCATCGCCTTCG | |
| | GCGGCTGCGTGTTCAGCTACGTGGGCTGCCACAACAAGTGCGCCTACT | |
| | GGGTGCCCAGGGCCAGCGCCAACATCGGCTGCAACCACACCGGCGTG | |
| | GTGGGCGAGGGCAGCGAGGGCCTGAACGACAACCTGCTGGAGATCCT | |
| | GCAGAAGGAGAAGGTGAACATCAACATCGTGGGCGACTTCAAGCTGAA | |
| | CGAGGAGATCGCCATCATCCTGGCCAGCTTCAGCGCCAGCACCAGCG | |
| | CCTTCGTGGAGACCGTGAAGGGCCTGGACTACAAGGCCTTCAAGCAGA | |
| | TCGTGGAGAGCTGCGGCAACTTCAAGGTGACCAAGGGCAAGGCCAAG | |
| | AAGGGCGCCTGGAACATCGGCGAGCAGAAGAGCATCCTGAGCCCCCT | |
| | GTACGCCTTCGCCAGCGAGGCCGCCAGGGTGGTGAGGAGCATCTTCA | |
| | GCAGGACCCTGGAGACCGCCCAGAACAGCGTGAGGGTGCTGCAGAAG | |
| | GCCGCCATCACCATCCTGGACGGCATCAGCCAGTACAGCCTGAGGCTG | |
| | ATCGACGCCATGATGTTCACCAGCGACCTGGCCACCAACAACCTGGTG | |
| | GTGATGGCCTACATCACCGGCGGCGTGGTGCAGCTGACCAGCCAGTG | |
| | GCTGACCAACATCTTCGGCACCGTGTACGAGAAGCTGAAGCCCGTGCT | |
| | GGACTGGCTGGAGGAGAAGTTCAAGGAGGGCGTGGAGTTCCTGAGGG | |
| | ACGGCTGGGAGATCGTGAAGTTCATCAGCACCTGCGCCTGCGAGATCG | |
| | TGGGCGGCCAGATCGTGACCTGCGCCAAGGAGATCAAGGAGAGCGTG | |
| | CAGACCTTCTTCAAGCTGGTGAACAAGTTCCTGGCCCTGTGCGCCGAC | |
| | AGCATCATCATCGGCGGCGCCAAGCTGAAGGCCCTGAACCTGGGCGA | |
| | GACCTTCGTGACCCACAGCAAGGGCCTGTACAGGAAGTGCGTGAAGAG | |
| | CAGGGAGGAGACCGGCCTGCTGATGCCCCTGAAGGCCCCCAAGGAGA | |
| | TCATCTTCCTGGAGGGCGAGACCCTGCCCACCGAGGTGCTGACCGAG | |
| | GAGGTGGTGCTGAAGACCGGCGACCTGCAGCCCCTGGAGCAGCCCAC | |
| | CAGCGAGGCCGTGGAGGCCCCCCTGGTGGGCACCCCCGTGTGCATCA | |
| | ACGGCCTGATGCTGCTGGAGATCAAGGACACCGAGAAGTACTGCGCCC | |
| | TGGCCCCCAACATGATGGTGACCAACAACACCTTCACCCTGAAGGGCG | |
| | GCTGATGACTCGAGCTGGTACTGCATGCACGCAATGCTAGCTGCCCCT | |
| | TTCCCGTCCTGGGTACCCCGAGTCTCCCCCGACCTCGGGTCCCAGGTA | |
| | TGCTCCCACCTCCACCTGCCCCACTCACCACCTCTGCTAGTTCCAGACA | |
| | CCTCCCAAGCACGCAGCAATGCAGCTCAAAACGCTTAGCCTAGCCACA | |
| | CCCCCACGGGAAACAGCAGTGATTAACCTTTAGCAATAAACGAAAGTTT | |
| | AACTAAGCTATACTAACCCCAGGGTTGGTCAATTTCGTGCCAGCCACAC | |
| | CCTGGAGCTAGCAAAAAAAA | |
| 11 | TAGTTATTAATAGTAATCAATTACGGGGTCATTAGTTCATAGCCCATATA | 296 |
| Ad5-Pan-CoV | TGGAGTTCCGCGTTACATAACTTACGGTAAATGGCCCGCCTGGCTGAC | |
| HexaPro- | CGCCCAACGACCCCCGCCCATTGACGTCAATAATGACGTATGTTCCCAT | |
| NSP1-NSP2 | AGTAACGCCAATAGGGACTTTCCATTGACGTCAATGGGTGGAGTATTTA | |
| | CGGTAAACTGCCCACTTGGCAGTACATCAAGTGTATCATATGCCAAGTA | |
| | CGCCCCCTATTGACGTCAATGACGGTAAATGGCCCGCCTGGCATTATG | |
| | CCCAGTACATGACCTTATGGGACTTTCCTACTTGGCAGTACATCTACGT | |
| | ATTAGTCATCGCTATTACCATGGTGATGCGGTTTTGGCAGTACATCAAT | |
| | GGGCGTGGATAGCGGTTTGACTCACGGGGATTTCCAAGTCTCCACCCC | |
| | ATTGACGTCAATGGGAGTTTGTTTTGGCACCAAAATCAACGGGACTTTC | |
| | CAAAATGTCGTAACAACTCCGCCCCATTGACGCAAATGGGCGGTAGGC | |
| | GTGTACGGTGGGAGGTCTATATAAGCAGAGCTGGTTTAGTGAACCGTC | |
| | AGATCGAGAATAAACTAGTATTCTTCTGGTCCCCACAGACTCAGAGAGA | |
| | ACCCGCCACCATGTTCGTGTTCCTGGTGCTGCTGCCCCTGGTGAGCAG | |
| | CCAGTGCGTGAACCTGACCACCAGGACCCAGCTGCCCCCCGCCTACA | |
| | CCAACAGCTTCACCAGGGGCGTGTACTACCCCGACAAGGTGTTCAGGA | |
| | GCAGCGTGCTGCACAGCACCCAGGACCTGTTCCTGCCCTTCTTCAGCA | |
| | ACGTGACCTGGTTCCACGCCATCCACGTGAGCGGCACCAACGGCACCA | |
| | AGAGGTTCGACAACCCCGTGCTGCCCTTCAACGACGGCGTGTACTTCG | |
| | CCAGCACCGAGAAGAGCAACATCATCAGGGGCTGGATCTTCGGCACCA | |
| | CCCTGGACAGCAAGACCCAGAGCCTGCTGATCGTGAACAACGCCACCA | |
| | ACGTGGTGATCAAGGTGTGCGAGTTCCAGTTCTGCAACGACCCCTTCC | |
| | TGGGCGTGTACTACCACAAGAACAACAAGAGCTGGATGGAGAGCGAGT | |
| | TCAGGGTGTACAGCAGCGCCAACAACTGCACCTTCGAGTACGTGAGCC | |
| | AGCCCTTCCTGATGGACCTGGAGGGCAAGCAGGGCAACTTCAAGAACC | |
| | TGAGGGAGTTCGTGTTCAAGAACATCGACGGCTACTTCAAGATCTACAG | |
| | CAAGCACACCCCCATCAACCTGGTGAGGGACCTGCCCCAGGGCTTCAG | |
| | CGCCCTGGAGCCCCTGGTGGACCTGCCCATCGGCATCAACATCACCAG | |
| | GTTCCAGACCCTGCTGGCCCTGCACAGGAGCTACCTGACCCCCGGCG | |
| | ACAGCAGCAGCGGCTGGACCGCCGGCGCCGCCGCCTACTACGTGGGC | |
| | TACCTGCAGCCCAGGACCTTCCTGCTGAAGTACAACGAGAACGGCACC | |
| | ATCACCGACGCCGTGGACTGCGCCCTGGACCCCCTGAGCGAGACCAA | |
| | GTGCACCCTGAAGAGCTTCACCGTGGAGAAGGGCATCTACCAGACCAG | |
| | CAACTTCAGGGTGCAGCCCACCGAGAGCATCGTGAGGTTCCCCAACAT | |
| | CACCAACCTGTGCCCCTTCGGCGAGGTGTTCAACGCCACCAGGTTCGC | |
| | CAGCGTGTACGCCTGGAACAGGAAGAGGATCAGCAACTGCGTGGCCG | |
| | ACTACAGCGTGCTGTACAACAGCGCCAGCTTCAGCACCTTCAAGTGCT | |
| | ACGGCGTGAGCCCCACCAAGCTGAACGACCTGTGCTTCACCAACGTGT | |
| | ACGCCGACAGCTTCGTGATCAGGGGCGACGAGGTGAGGCAGATCGCC | |
| | CCCGGCCAGACCGGCAAGATCGCCGACTACAACTACAAGCTGCCCGA | |
| | CGACTTCACCGGCTGCGTGATCGCCTGGAACAGCAACAACCTGGACAG | |
| | CAAGGTGGGCGGCAACTACAACTACCTGTACAGGCTGTTCAGGAAGAG | |
| | CAACCTGAAGCCCTTCGAGAGGGACATCAGCACCGAGATCTACCAGGC | |
| | CGGCAGCACCCCCTGCAACGGCGTGGAGGGCTTCAACTGCTACTTCCC | |
| | CCTGCAGAGCTACGGCTTCCAGCCCACCAACGGCGTGGGCTACCAGC | |
| | CCTACAGGGTGGTGGTGCTGAGCTTCGAGCTGCTGCACGCCCCCGCC | |
| | ACCGTGTGCGGCCCCAAGAAGAGCACCAACCTGGTGAAGAACAAGTGC | |
| | GTGAACTTCAACTTCAACGGCCTGACCGGCACCGGCGTGCTGACCGAG | |
| | AGCAACAAGAAGTTCCTGCCCTTCCAGCAGTTCGGCAGGGACATCGCC | |
| | GACACCACCGACGCCGTGAGGGACCCCCAGACCCTGGAGATCCTGGA | |
| | CATCACCCCCTGCAGCTTCGGCGGCGTGAGCGTGATCACCCCCGGCA | |
| | CCAACACCAGCAACCAGGTGGCCGTGCTGTACCAGGACGTGAACTGCA | |
| | CCGAGGTGCCCGTGGCCATCCACGCCGACCAGCTGACCCCCACCTGG | |
| | AGGGTGTACAGCACCGGCAGCAACGTGTTCCAGACCAGGGCCGGCTG | |
| | CCTGATCGGCGCCGAGCACGTGAACAACAGCTACGAGTGCGACATCCC | |
| | CATCGGCGCCGGCATCTGCGCCAGCTACCAGACCCAGACCAACAGCC | |
| | CCGGCAGCGCCAGCAGCGTGGCCAGCCAGAGCATCATCGCCTACACC | |
| | ATGAGCCTGGGCGCCGAGAACAGCGTGGCCTACAGCAACAACAGCAT | |
| | CGCCATCCCCACCAACTTCACCATCAGCGTGACCACCGAGATCCTGCC | |
| | CGTGAGCATGACCAAGACCAGCGTGGACTGCACCATGTACATCTGCGG | |
| | CGACAGCACCGAGTGCAGCAACCTGCTGCTGCAGTACGGCAGCTTCTG | |
| | CACCCAGCTGAACAGGGCCCTGACCGGCATCGCCGTGGAGCAGGACA | |
| | AGAACACCCAGGAGGTGTTCGCCCAGGTGAAGCAGATCTACAAGACCC | |
| | CCCCCATCAAGGACTTCGGCGGCTTCAACTTCAGCCAGATCCTGCCCG | |
| | ACCCCAGCAAGCCCAGCAAGAGGAGCCCCATCGAGGACCTGCTGTTCA | |
| | ACAAGGTGACCCTGGCCGACGCCGGCTTCATCAAGCAGTACGGCGACT | |
| | GCCTGGGCGACATCGCCGCCAGGGACCTGATCTGCGCCCAGAAGTTC | |
| | AACGGCCTGACCGTGCTGCCCCCCCTGCTGACCGACGAGATGATCGC | |
| | CCAGTACACCAGCGCCCTGCTGGCCGGCACCATCACCAGCGGCTGGA | |
| | CCTTCGGCGCCGGCCCCGCCCTGCAGATCCCCTTCCCCATGCAGATG | |
| | GCCTACAGGTTCAACGGCATCGGCGTGACCCAGAACGTGCTGTACGAG | |
| | AACCAGAAGCTGATCGCCAACCAGTTCAACAGCGCCATCGGCAAGATC | |
| | CAGGACAGCCTGAGCAGCACCCCCAGCGCCCTGGGCAAGCTGCAGGA | |
| | CGTGGTGAACCAGAACGCCCAGGCCCTGAACACCCTGGTGAAGCAGC | |
| | TGAGCAGCAACTTCGGCGCCATCAGCAGCGTGCTGAACGACATCCTGA | |
| | GCAGGCTGGACCCCCCCGAGGCCGAGGTGCAGATCGACAGGCTGATC | |
| | ACCGGCAGGCTGCAGAGCCTGCAGACCTACGTGACCCAGCAGCTGAT | |
| | CAGGGCCGCCGAGATCAGGGCCAGCGCCAACCTGGCCGCCACCAAGA | |
| | TGAGCGAGTGCGTGCTGGGCCAGAGCAAGAGGGTGGACTTCTGCGGC | |
| | AAGGGCTACCACCTGATGAGCTTCCCCCAGAGCGCCCCCCACGGCGT | |
| | GGTGTTCCTGCACGTGACCTACGTGCCCGCCCAGGAGAAGAACTTCAC | |
| | CACCGCCCCCGCCATCTGCCACGACGGCAAGGCCCACTTCCCCAGGG | |
| | AGGGCGTGTTCGTGAGCAACGGCACCCACTGGTTCGTGACCCAGAGG | |
| | AACTTCTACGAGCCCCAGATCATCACCACCGACAACACCTTCGTGAGC | |
| | GGCAACTGCGACGTGGTGATCGGCATCGTGAACAACACCGTGTACGAC | |
| | CCCCTGCAGCCCGAGCTGGACAGCTTCAAGGAGGAGCTGGACAAGTA | |
| | CTTCAAGAACCACACCAGCCCCGACGTGGACCTGGGCGACATCAGCG | |
| | GCATCAACGCCAGCGTGGTGAACATCCAGAAGGAGATCGACAGGCTGA | |
| | ACGAGGTGGCCAAGAACCTGAACGAGAGCCTGATCGACCTGCAGGAG | |
| | CTGGGCAAGTACGAGCAGTACATCAAGTGGCCCTGGTACATCTGGCTG | |
| | GGCTTCATCGCCGGCCTGATCGCCATCGTGATGGTGACCATCATGCTG | |
| | TGCTGCATGACCAGCTGCTGCAGCTGCCTGAAGGGCTGCTGCAGCTGC | |
| | GGCAGCTGCTGCAAGTTCGACGAGGACGACAGCGAGCCCGTGCTGAA | |
| | GGGCGTGAAGCTGCACTACACCGGAAGCGGAGCCACGAACTTCTCTCT | |
| | GTTAAAGCAAGCAGGAGATGTTGAAGAAAACCCCGGGCCTATGGAGAG | |
| | CCTGGTGCCCGGCTTCAACGAGAAGACCCACGTGCAGCTGAGCCTGC | |
| | CCGTGCTGCAGGTGAGGGACGTGCTGGTGAGGGGCTTCGGCGACAGC | |
| | GTGGAGGAGGTGCTGAGCGAGGCCAGGCAGCACCTGAAGGACGGCAC | |
| | CTGCGGCCTGGTGGAGGTGGAGAAGGGCGTGCTGCCCCAGCTGGAGC | |
| | AGCCCTACGTGTTCATCAAGAGGAGCGACGCCAGGACCGCCCCCCAC | |
| | GGCCACGTGATGGTGGAGCTGGTGGCCGAGCTGGAGGGCATCCAGTA | |
| | CGGCAGGAGCGGCGAGACCCTGGGCGTGCTGGTGCCCCACGTGGGC | |
| | GAGATCCCCGTGGCCTACAGGAAGGTGCTGCTGAGGAAGAACGGCAA | |
| | CAAGGGCGCCGGCGGCCACAGCTACGGCGCCGACCTGAAGAGCTTCG | |
| | ACCTGGGCGACGAGCTGGGCACCGACCCCTACGAGGACTTCCAGGAG | |
| | AACTGGAACACCAAGCACAGCAGCGGCGTGACCAGGGAGCTGATGAG | |
| | GGAGCTGAACGGCGGCGGAAGCGGAGAGGGCAGGGGAAGTCTTCTAA | |
| | CATGCGGGGACGTGGAGGAAAATCCCGGCCCCGCCTACACCAGGTAC | |
| | GTGGACAACAACTTCTGCGGCCCCGACGGCTACCCCCTGGAGTGCATC | |
| | AAGGACCTGCTGGCCAGGGCCGGCAAGGCCAGCTGCACCCTGAGCGA | |
| | GCAGCTGGACTTCATCGACACCAAGAGGGGCGTGTACTGCTGCAGGG | |
| | AGCACGAGCACGAGATCGCCTGGTACACCGAGAGGAGCGAGAAGAGC | |
| | TACGAGCTGCAGACCCCCTTCGAGATCAAGCTGGCCAAGAAGTTCGAC | |
| | ACCTTCAACGGCGAGTGCCCCAACTTCGTGTTCCCCCTGAACAGCATC | |
| | ATCAAGACCATCCAGCCCAGGGTGGAGAAGAAGAAGCTGGACGGCTTC | |
| | ATGGGCAGGATCAGGAGCGTGTACCCCGTGGCCAGCCCCAACGAGTG | |
| | CAACCAGATGTGCCTGAGCACCCTGATGAAGTGCGACCACTGCGGCGA | |
| | GACCAGCTGGCAGACCGGCGACTTCGTGAAGGCCACCTGCGAGTTCT | |
| | GCGGCACCGAGAACCTGACCAAGGAGGGCGCCACCACCTGCGGCTAC | |
| | CTGCCCCAGAACGCCGTGGTGAAGATCTACTGCCCCGCCTGCCACAAC | |
| | AGCGAGGTGGGCCCCGAGCACAGCCTGGCCGAGTACCACAACGAGAG | |
| | CGGCCTGAAGACCATCCTGAGGAAGGGCGGCAGGACCATCGCCTTCG | |
| | GCGGCTGCGTGTTCAGCTACGTGGGCTGCCACAACAAGTGCGCCTACT | |
| | GGGTGCCCAGGGCCAGCGCCAACATCGGCTGCAACCACACCGGCGTG | |
| | GTGGGCGAGGGCAGCGAGGGCCTGAACGACAACCTGCTGGAGATCCT | |
| | GCAGAAGGAGAAGGTGAACATCAACATCGTGGGCGACTTCAAGCTGAA | |
| | CGAGGAGATCGCCATCATCCTGGCCAGCTTCAGCGCCAGCACCAGCG | |
| | CCTTCGTGGAGACCGTGAAGGGCCTGGACTACAAGGCCTTCAAGCAGA | |
| | TCGTGGAGAGCTGCGGCAACTTCAAGGTGACCAAGGGCAAGGCCAAG | |
| | AAGGGCGCCTGGAACATCGGCGAGCAGAAGAGCATCCTGAGCCCCCT | |
| | GTACGCCTTCGCCAGCGAGGCCGCCAGGGTGGTGAGGAGCATCTTCA | |
| | GCAGGACCCTGGAGACCGCCCAGAACAGCGTGAGGGTGCTGCAGAAG | |
| | GCCGCCATCACCATCCTGGACGGCATCAGCCAGTACAGCCTGAGGCTG | |
| | ATCGACGCCATGATGTTCACCAGCGACCTGGCCACCAACAACCTGGTG | |
| | GTGATGGCCTACATCACCGGCGGCGTGGTGCAGCTGACCAGCCAGTG | |
| | GCTGACCAACATCTTCGGCACCGTGTACGAGAAGCTGAAGCCCGTGCT | |
| | GGACTGGCTGGAGGAGAAGTTCAAGGAGGGCGTGGAGTTCCTGAGGG | |
| | ACGGCTGGGAGATCGTGAAGTTCATCAGCACCTGCGCCTGCGAGATCG | |
| | TGGGCGGCCAGATCGTGACCTGCGCCAAGGAGATCAAGGAGAGCGTG | |
| | CAGACCTTCTTCAAGCTGGTGAACAAGTTCCTGGCCCTGTGCGCCGAC | |
| | AGCATCATCATCGGCGGCGCCAAGCTGAAGGCCCTGAACCTGGGCGA | |
| | GACCTTCGTGACCCACAGCAAGGGCCTGTACAGGAAGTGCGTGAAGAG | |
| | CAGGGAGGAGACCGGCCTGCTGATGCCCCTGAAGGCCCCCAAGGAGA | |
| | TCATCTTCCTGGAGGGCGAGACCCTGCCCACCGAGGTGCTGACCGAG | |
| | GAGGTGGTGCTGAAGACCGGCGACCTGCAGCCCCTGGAGCAGCCCAC | |
| | CAGCGAGGCCGTGGAGGCCCCCCTGGTGGGCACCCCCGTGTGCATCA | |
| | ACGGCCTGATGCTGCTGGAGATCAAGGACACCGAGAAGTACTGCGCCC | |
| | TGGCCCCCAACATGATGGTGACCAACAACACCTTCACCCTGAAGGGCG | |
| | GCTGATGACTCGAGCTGGTACTGCATGCACGCAATGCTAGCTGCCCCT | |
| | TTCCCGTCCTGGGTACCCCGAGTCTCCCCCGACCTCGGGTCCCAGGTA | |
| | TGCTCCCACCTCCACCTGCCCCACTCACCACCTCTGCTAGTTCCAGACA | |
| | CCTCCCAAGCACGCAGCAATGCAGCTCAAAACGCTTAGCCTAGCCACA | |
| | CCCCCACGGGAAACAGCAGTGATTAACCTTTAGCAATAAACGAAAGTTT | |
| | AACTAAGCTATACTAACCCCAGGGTTGGTCAATTTCGTGCCAGCCACAC | |
| | CCTGGAGCTAGCAAAAAAAA | |
| 12 | TAGTTATTAATAGTAATCAATTACGGGGTCATTAGTTCATAGCCCATATA | 237 |
| Ad5-Pan-CoV | TGGAGTTCCGCGTTACATAACTTACGGTAAATGGCCCGCCTGGCTGAC | |
| Spike-NSP6- | CGCCCAACGACCCCCGCCCATTGACGTCAATAATGACGTATGTTCCCAT | |
| NSP7-NSP8 | AGTAACGCCAATAGGGACTTTCCATTGACGTCAATGGGTGGAGTATTTA | |
| | CGGTAAACTGCCCACTTGGCAGTACATCAAGTGTATCATATGCCAAGTA | |
| | CGCCCCCTATTGACGTCAATGACGGTAAATGGCCCGCCTGGCATTATG | |
| | CCCAGTACATGACCTTATGGGACTTTCCTACTTGGCAGTACATCTACGT | |
| | ATTAGTCATCGCTATTACCATGGTGATGCGGTTTTGGCAGTACATCAAT | |
| | GGGCGTGGATAGCGGTTTGACTCACGGGGATTTCCAAGTCTCCACCCC | |
| | ATTGACGTCAATGGGAGTTTGTTTTGGCACCAAAATCAACGGGACTTTC | |
| | CAAAATGTCGTAACAACTCCGCCCCATTGACGCAAATGGGCGGTAGGC | |
| | GTGTACGGTGGGAGGTCTATATAAGCAGAGCTGGTTTAGTGAACCGTC | |
| | AGATCGAGAATAAACTAGTATTCTTCTGGTCCCCACAGACTCAGAGAGA | |
| | ACCCGCCACCATGTTCGTGTTCCTGGTGCTGCTGCCCCTGGTGAGCAG | |
| | CCAGTGCGTGAACCTGACCACCAGGACCCAGCTGCCCCCCGCCTACA | |
| | CCAACAGCTTCACCAGGGGCGTGTACTACCCCGACAAGGTGTTCAGGA | |
| | GCAGCGTGCTGCACAGCACCCAGGACCTGTTCCTGCCCTTCTTCAGCA | |
| | ACGTGACCTGGTTCCACGCCATCCACGTGAGCGGCACCAACGGCACCA | |
| | AGAGGTTCGACAACCCCGTGCTGCCCTTCAACGACGGCGTGTACTTCG | |
| | CCAGCACCGAGAAGAGCAACATCATCAGGGGCTGGATCTTCGGCACCA | |
| | CCCTGGACAGCAAGACCCAGAGCCTGCTGATCGTGAACAACGCCACCA | |
| | ACGTGGTGATCAAGGTGTGCGAGTTCCAGTTCTGCAACGACCCCTTCC | |
| | TGGGCGTGTACTACCACAAGAACAACAAGAGCTGGATGGAGAGCGAGT | |
| | TCAGGGTGTACAGCAGCGCCAACAACTGCACCTTCGAGTACGTGAGCC | |
| | AGCCCTTCCTGATGGACCTGGAGGGCAAGCAGGGCAACTTCAAGAACC | |
| | TGAGGGAGTTCGTGTTCAAGAACATCGACGGCTACTTCAAGATCTACAG | |
| | CAAGCACACCCCCATCAACCTGGTGAGGGACCTGCCCCAGGGCTTCAG | |
| | CGCCCTGGAGCCCCTGGTGGACCTGCCCATCGGCATCAACATCACCAG | |
| | GTTCCAGACCCTGCTGGCCCTGCACAGGAGCTACCTGACCCCCGGCG | |
| | ACAGCAGCAGCGGCTGGACCGCCGGCGCCGCCGCCTACTACGTGGGC | |
| | TACCTGCAGCCCAGGACCTTCCTGCTGAAGTACAACGAGAACGGCACC | |
| | ATCACCGACGCCGTGGACTGCGCCCTGGACCCCCTGAGCGAGACCAA | |
| | GTGCACCCTGAAGAGCTTCACCGTGGAGAAGGGCATCTACCAGACCAG | |
| | CAACTTCAGGGTGCAGCCCACCGAGAGCATCGTGAGGTTCCCCAACAT | |
| | CACCAACCTGTGCCCCTTCGGCGAGGTGTTCAACGCCACCAGGTTCGC | |
| | CAGCGTGTACGCCTGGAACAGGAAGAGGATCAGCAACTGCGTGGCCG | |
| | ACTACAGCGTGCTGTACAACAGCGCCAGCTTCAGCACCTTCAAGTGCT | |
| | ACGGCGTGAGCCCCACCAAGCTGAACGACCTGTGCTTCACCAACGTGT | |
| | ACGCCGACAGCTTCGTGATCAGGGGCGACGAGGTGAGGCAGATCGCC | |
| | CCCGGCCAGACCGGCAAGATCGCCGACTACAACTACAAGCTGCCCGA | |
| | CGACTTCACCGGCTGCGTGATCGCCTGGAACAGCAACAACCTGGACAG | |
| | CAAGGTGGGCGGCAACTACAACTACCTGTACAGGCTGTTCAGGAAGAG | |
| | CAACCTGAAGCCCTTCGAGAGGGACATCAGCACCGAGATCTACCAGGC | |
| | CGGCAGCACCCCCTGCAACGGCGTGGAGGGCTTCAACTGCTACTTCCC | |
| | CCTGCAGAGCTACGGCTTCCAGCCCACCAACGGCGTGGGCTACCAGC | |
| | CCTACAGGGTGGTGGTGCTGAGCTTCGAGCTGCTGCACGCCCCCGCC | |
| | ACCGTGTGCGGCCCCAAGAAGAGCACCAACCTGGTGAAGAACAAGTGC | |
| | GTGAACTTCAACTTCAACGGCCTGACCGGCACCGGCGTGCTGACCGAG | |
| | AGCAACAAGAAGTTCCTGCCCTTCCAGCAGTTCGGCAGGGACATCGCC | |
| | GACACCACCGACGCCGTGAGGGACCCCCAGACCCTGGAGATCCTGGA | |
| | CATCACCCCCTGCAGCTTCGGCGGCGTGAGCGTGATCACCCCCGGCA | |
| | CCAACACCAGCAACCAGGTGGCCGTGCTGTACCAGGACGTGAACTGCA | |
| | CCGAGGTGCCCGTGGCCATCCACGCCGACCAGCTGACCCCCACCTGG | |
| | AGGGTGTACAGCACCGGCAGCAACGTGTTCCAGACCAGGGCCGGCTG | |
| | CCTGATCGGCGCCGAGCACGTGAACAACAGCTACGAGTGCGACATCCC | |
| | CATCGGCGCCGGCATCTGCGCCAGCTACCAGACCCAGACCAACAGCC | |
| | CCAGGAGGGCCAGGAGCGTGGCCAGCCAGAGCATCATCGCCTACACC | |
| | ATGAGCCTGGGCGCCGAGAACAGCGTGGCCTACAGCAACAACAGCAT | |
| | CGCCATCCCCACCAACTTCACCATCAGCGTGACCACCGAGATCCTGCC | |
| | CGTGAGCATGACCAAGACCAGCGTGGACTGCACCATGTACATCTGCGG | |
| | CGACAGCACCGAGTGCAGCAACCTGCTGCTGCAGTACGGCAGCTTCTG | |
| | CACCCAGCTGAACAGGGCCCTGACCGGCATCGCCGTGGAGCAGGACA | |
| | AGAACACCCAGGAGGTGTTCGCCCAGGTGAAGCAGATCTACAAGACCC | |
| | CCCCCATCAAGGACTTCGGCGGCTTCAACTTCAGCCAGATCCTGCCCG | |
| | ACCCCAGCAAGCCCAGCAAGAGGAGCTTCATCGAGGACCTGCTGTTCA | |
| | ACAAGGTGACCCTGGCCGACGCCGGCTTCATCAAGCAGTACGGCGACT | |
| | GCCTGGGCGACATCGCCGCCAGGGACCTGATCTGCGCCCAGAAGTTC | |
| | AACGGCCTGACCGTGCTGCCCCCCCTGCTGACCGACGAGATGATCGC | |
| | CCAGTACACCAGCGCCCTGCTGGCCGGCACCATCACCAGCGGCTGGA | |
| | CCTTCGGCGCCGGCGCCGCCCTGCAGATCCCCTTCGCCATGCAGATG | |
| | GCCTACAGGTTCAACGGCATCGGCGTGACCCAGAACGTGCTGTACGAG | |
| | AACCAGAAGCTGATCGCCAACCAGTTCAACAGCGCCATCGGCAAGATC | |
| | CAGGACAGCCTGAGCAGCACCGCCAGCGCCCTGGGCAAGCTGCAGGA | |
| | CGTGGTGAACCAGAACGCCCAGGCCCTGAACACCCTGGTGAAGCAGC | |
| | TGAGCAGCAACTTCGGCGCCATCAGCAGCGTGCTGAACGACATCCTGA | |
| | GCAGGCTGGACCCCCCCGAGGCCGAGGTGCAGATCGACAGGCTGATC | |
| | ACCGGCAGGCTGCAGAGCCTGCAGACCTACGTGACCCAGCAGCTGAT | |
| | CAGGGCCGCCGAGATCAGGGCCAGCGCCAACCTGGCCGCCACCAAGA | |
| | TGAGCGAGTGCGTGCTGGGCCAGAGCAAGAGGGTGGACTTCTGCGGC | |
| | AAGGGCTACCACCTGATGAGCTTCCCCCAGAGCGCCCCCCACGGCGT | |
| | GGTGTTCCTGCACGTGACCTACGTGCCCGCCCAGGAGAAGAACTTCAC | |
| | CACCGCCCCCGCCATCTGCCACGACGGCAAGGCCCACTTCCCCAGGG | |
| | AGGGCGTGTTCGTGAGCAACGGCACCCACTGGTTCGTGACCCAGAGG | |
| | AACTTCTACGAGCCCCAGATCATCACCACCGACAACACCTTCGTGAGC | |
| | GGCAACTGCGACGTGGTGATCGGCATCGTGAACAACACCGTGTACGAC | |
| | CCCCTGCAGCCCGAGCTGGACAGCTTCAAGGAGGAGCTGGACAAGTA | |
| | CTTCAAGAACCACACCAGCCCCGACGTGGACCTGGGCGACATCAGCG | |
| | GCATCAACGCCAGCGTGGTGAACATCCAGAAGGAGATCGACAGGCTGA | |
| | ACGAGGTGGCCAAGAACCTGAACGAGAGCCTGATCGACCTGCAGGAG | |
| | CTGGGCAAGTACGAGCAGTACATCAAGTGGCCCTGGTACATCTGGCTG | |
| | GGCTTCATCGCCGGCCTGATCGCCATCGTGATGGTGACCATCATGCTG | |
| | TGCTGCATGACCAGCTGCTGCAGCTGCCTGAAGGGCTGCTGCAGCTGC | |
| | GGCAGCTGCTGCAAGTTCGACGAGGACGACAGCGAGCCCGTGCTGAA | |
| | GGGCGTGAAGCTGCACTACACCGGAAGCGGAGCCACGAACTTCTCTCT | |
| | GTTAAAGCAAGCAGGAGATGTTGAAGAAAACCCCGGGCCTAGCGCCGT | |
| | GAAGAGGACCATCAAGGGCACCCACCACTGGCTGCTGCTGACCATCCT | |
| | GACCAGCCTGCTGGTGCTGGTGCAGAGCACCCAGTGGAGCCTGTTCTT | |
| | CTTCCTGTACGAGAACGCCTTCCTGCCCTTCGCCATGGGCATCATCGC | |
| | CATGAGCGCCTTCGCCATGATGTTCGTGAAGCACAAGCACGCCTTCCT | |
| | GTGCCTGTTCCTGCTGCCCAGCCTGGCCACCGTGGCCTACTTCAACAT | |
| | GGTGTACATGCCCGCCAGCTGGGTGATGAGGATCATGACCTGGCTGGA | |
| | CATGGTGGACACCAGCCTGAGCGGCTTCAAGCTGAAGGACTGCGTGAT | |
| | GTACGCCAGCGCCGTGGTGCTGCTGATCCTGATGACCGCCAGGACCG | |
| | TGTACGACGACGGCGCCAGGAGGGTGTGGACCCTGATGAACGTGCTG | |
| | ACCCTGGTGTACAAGGTGTACTACGGCAACGCCCTGGACCAGGCCATC | |
| | AGCATGTGGGCCCTGATCATCAGCGTGACCAGCAACTACAGCGGCGTG | |
| | GTGACCACCGTGATGTTCCTGGCCAGGGGCATCGTGTTCATGTGCGTG | |
| | GAGTACTGCCCCATCTTCTTCATCACCGGCAACACCCTGCAGTGCATCA | |
| | TGCTGGTGTACTGCTTCCTGGGCTACTTCTGCACCTGCTACTTCGGCCT | |
| | GTTCTGCCTGCTGAACAGGTACTTCAGGCTGACCCTGGGCGTGTACGA | |
| | CTACCTGGTGAGCACCCAGGAGTTCAGGTACATGAACAGCCAGGGCCT | |
| | GCTGCCCCCCAAGAACAGCATCGACGCCTTCAAGCTGAACATCAAGCT | |
| | GCTGGGCGTGGGCGGCAAGCCCTGCATCAAGGTGGCCACCGTGCAGG | |
| | GAAGCGGAGAGGGCAGGGGAAGTCTTCTAACATGCGGGGACGTGGAG | |
| | GAAAATCCCGGCCCCAGCAAGATGAGCGACGTGAAGTGCACCAGCGT | |
| | GGTGCTGCTGAGCGTGCTGCAGCAGCTGAGGGTGGAGAGCAGCAGCA | |
| | AGCTGTGGGCCCAGTGCGTGCAGCTGCACAACGACATCCTGCTGGCC | |
| | AAGGACACCACCGAGGCCTTCGAGAAGATGGTGAGCCTGCTGAGCGT | |
| | GCTGCTGAGCATGCAGGGCGCCGTGGACATCAACAAGCTGTGCGAGG | |
| | AGATGCTGGACAACAGGGCCACCCTGCAGGGAAGCGGACAGTGTACT | |
| | AATTATGCTCTCTTGAAATTGGCTGGAGATGTTGAGAGCAACCCAGGTC | |
| | CCGCCATCGCCAGCGAGTTCAGCAGCCTGCCCAGCTACGCCGCCTTC | |
| | GCCACCGCCCAGGAGGCCTACGAGCAGGCCGTGGCCAACGGCGACA | |
| | GCGAGGTGGTGCTGAAGAAGCTGAAGAAGAGCCTGAACGTGGCCAAG | |
| | AGCGAGTTCGACAGGGACGCCGCCATGCAGAGGAAGCTGGAGAAGAT | |
| | GGCCGACCAGGCCATGACCCAGATGTACAAGCAGGCCAGGAGCGAGG | |
| | ACAAGAGGGCCAAGGTGACCAGCGCCATGCAGACCATGCTGTTCACCA | |
| | TGCTGAGGAAGCTGGACAACGACGCCCTGAACAACATCATCAACAACG | |
| | CCAGGGACGGCTGCGTGCCCCTGAACATCATCCCCCTGACCACCGCC | |
| | GCCAAGCTGATGGTGGTGATCCCCGACTACAACACCTACAAGAACACC | |
| | TGCGACGGCACCACCTTCACCTACGCCAGCGCCCTGTGGGAGATCCA | |
| | GCAGGTGGTGGACGCCGACAGCAAGATCGTGCAGCTGAGCGAGATCA | |
| | GCATGGACAACAGCCCCAACCTGGCCTGGCCCCTGATCGTGACCGCC | |
| | CTGAGGGCCAACAGCGCCGTGAAGCTGCAGTGATGACTCGAGCTGGT | |
| | ACTGCATGCACGCAATGCTAGCTGCCCCTTTCCCGTCCTGGGTACCCC | |
| | GAGTCTCCCCCGACCTCGGGTCCCAGGTATGCTCCCACCTCCACCTGC | |
| | CCCACTCACCACCTCTGCTAGTTCCAGACACCTCCCAAGCACGCAGCA | |
| | ATGCAGCTCAAAACGCTTAGCCTAGCCACACCCCCACGGGAAACAGCA | |
| | GTGATTAACCTTTAGCAATAAACGAAAGTTTAACTAAGCTATACTAACCC | |
| | CAGGGTTGGTCAATTTCGTGCCAGCCACACCCTGGAGCTAGCAAAAAA | |
| | AA | |
| 13 | TAGTTATTAATAGTAATCAATTACGGGGTCATTAGTTCATAGCCCATATA | 298 |
| Ad5-Pan-CoV | TGGAGTTCCGCGTTACATAACTTACGGTAAATGGCCCGCCTGGCTGAC | |
| HexaPro- | CGCCCAACGACCCCCGCCCATTGACGTCAATAATGACGTATGTTCCCAT | |
| NSP6-NSP7- | AGTAACGCCAATAGGGACTTTCCATTGACGTCAATGGGTGGAGTATTTA | |
| NSP8 | CGGTAAACTGCCCACTTGGCAGTACATCAAGTGTATCATATGCCAAGTA | |
| | CGCCCCCTATTGACGTCAATGACGGTAAATGGCCCGCCTGGCATTATG | |
| | CCCAGTACATGACCTTATGGGACTTTCCTACTTGGCAGTACATCTACGT | |
| | ATTAGTCATCGCTATTACCATGGTGATGCGGTTTTGGCAGTACATCAAT | |
| | GGGCGTGGATAGCGGTTTGACTCACGGGGATTTCCAAGTCTCCACCCC | |
| | ATTGACGTCAATGGGAGTTTGTTTTGGCACCAAAATCAACGGGACTTTC | |
| | CAAAATGTCGTAACAACTCCGCCCCATTGACGCAAATGGGCGGTAGGC | |
| | GTGTACGGTGGGAGGTCTATATAAGCAGAGCTGGTTTAGTGAACCGTC | |
| | AGATCGAGAATAAACTAGTATTCTTCTGGTCCCCACAGACTCAGAGAGA | |
| | ACCCGCCACCATGTTCGTGTTCCTGGTGCTGCTGCCCCTGGTGAGCAG | |
| | CCAGTGCGTGAACCTGACCACCAGGACCCAGCTGCCCCCCGCCTACA | |
| | CCAACAGCTTCACCAGGGGCGTGTACTACCCCGACAAGGTGTTCAGGA | |
| | GCAGCGTGCTGCACAGCACCCAGGACCTGTTCCTGCCCTTCTTCAGCA | |
| | ACGTGACCTGGTTCCACGCCATCCACGTGAGCGGCACCAACGGCACCA | |
| | AGAGGTTCGACAACCCCGTGCTGCCCTTCAACGACGGCGTGTACTTCG | |
| | CCAGCACCGAGAAGAGCAACATCATCAGGGGCTGGATCTTCGGCACCA | |
| | CCCTGGACAGCAAGACCCAGAGCCTGCTGATCGTGAACAACGCCACCA | |
| | ACGTGGTGATCAAGGTGTGCGAGTTCCAGTTCTGCAACGACCCCTTCC | |
| | TGGGCGTGTACTACCACAAGAACAACAAGAGCTGGATGGAGAGCGAGT | |
| | TCAGGGTGTACAGCAGCGCCAACAACTGCACCTTCGAGTACGTGAGCC | |
| | AGCCCTTCCTGATGGACCTGGAGGGCAAGCAGGGCAACTTCAAGAACC | |
| | TGAGGGAGTTCGTGTTCAAGAACATCGACGGCTACTTCAAGATCTACAG | |
| | CAAGCACACCCCCATCAACCTGGTGAGGGACCTGCCCCAGGGCTTCAG | |
| | CGCCCTGGAGCCCCTGGTGGACCTGCCCATCGGCATCAACATCACCAG | |
| | GTTCCAGACCCTGCTGGCCCTGCACAGGAGCTACCTGACCCCCGGCG | |
| | ACAGCAGCAGCGGCTGGACCGCCGGCGCCGCCGCCTACTACGTGGGC | |
| | TACCTGCAGCCCAGGACCTTCCTGCTGAAGTACAACGAGAACGGCACC | |
| | ATCACCGACGCCGTGGACTGCGCCCTGGACCCCCTGAGCGAGACCAA | |
| | GTGCACCCTGAAGAGCTTCACCGTGGAGAAGGGCATCTACCAGACCAG | |
| | CAACTTCAGGGTGCAGCCCACCGAGAGCATCGTGAGGTTCCCCAACAT | |
| | CACCAACCTGTGCCCCTTCGGCGAGGTGTTCAACGCCACCAGGTTCGC | |
| | CAGCGTGTACGCCTGGAACAGGAAGAGGATCAGCAACTGCGTGGCCG | |
| | ACTACAGCGTGCTGTACAACAGCGCCAGCTTCAGCACCTTCAAGTGCT | |
| | ACGGCGTGAGCCCCACCAAGCTGAACGACCTGTGCTTCACCAACGTGT | |
| | ACGCCGACAGCTTCGTGATCAGGGGCGACGAGGTGAGGCAGATCGCC | |
| | CCCGGCCAGACCGGCAAGATCGCCGACTACAACTACAAGCTGCCCGA | |
| | CGACTTCACCGGCTGCGTGATCGCCTGGAACAGCAACAACCTGGACAG | |
| | CAAGGTGGGCGGCAACTACAACTACCTGTACAGGCTGTTCAGGAAGAG | |
| | CAACCTGAAGCCCTTCGAGAGGGACATCAGCACCGAGATCTACCAGGC | |
| | CGGCAGCACCCCCTGCAACGGCGTGGAGGGCTTCAACTGCTACTTCCC | |
| | CCTGCAGAGCTACGGCTTCCAGCCCACCAACGGCGTGGGCTACCAGC | |
| | CCTACAGGGTGGTGGTGCTGAGCTTCGAGCTGCTGCACGCCCCCGCC | |
| | ACCGTGTGCGGCCCCAAGAAGAGCACCAACCTGGTGAAGAACAAGTGC | |
| | GTGAACTTCAACTTCAACGGCCTGACCGGCACCGGCGTGCTGACCGAG | |
| | AGCAACAAGAAGTTCCTGCCCTTCCAGCAGTTCGGCAGGGACATCGCC | |
| | GACACCACCGACGCCGTGAGGGACCCCCAGACCCTGGAGATCCTGGA | |
| | CATCACCCCCTGCAGCTTCGGCGGCGTGAGCGTGATCACCCCCGGCA | |
| | CCAACACCAGCAACCAGGTGGCCGTGCTGTACCAGGACGTGAACTGCA | |
| | CCGAGGTGCCCGTGGCCATCCACGCCGACCAGCTGACCCCCACCTGG | |
| | AGGGTGTACAGCACCGGCAGCAACGTGTTCCAGACCAGGGCCGGCTG | |
| | CCTGATCGGCGCCGAGCACGTGAACAACAGCTACGAGTGCGACATCCC | |
| | CATCGGCGCCGGCATCTGCGCCAGCTACCAGACCCAGACCAACAGCC | |
| | CCGGCAGCGCCAGCAGCGTGGCCAGCCAGAGCATCATCGCCTACACC | |
| | ATGAGCCTGGGCGCCGAGAACAGCGTGGCCTACAGCAACAACAGCAT | |
| | CGCCATCCCCACCAACTTCACCATCAGCGTGACCACCGAGATCCTGCC | |
| | CGTGAGCATGACCAAGACCAGCGTGGACTGCACCATGTACATCTGCGG | |
| | CGACAGCACCGAGTGCAGCAACCTGCTGCTGCAGTACGGCAGCTTCTG | |
| | CACCCAGCTGAACAGGGCCCTGACCGGCATCGCCGTGGAGCAGGACA | |
| | AGAACACCCAGGAGGTGTTCGCCCAGGTGAAGCAGATCTACAAGACCC | |
| | CCCCCATCAAGGACTTCGGCGGCTTCAACTTCAGCCAGATCCTGCCCG | |
| | ACCCCAGCAAGCCCAGCAAGAGGAGCCCCATCGAGGACCTGCTGTTCA | |
| | ACAAGGTGACCCTGGCCGACGCCGGCTTCATCAAGCAGTACGGCGACT | |
| | GCCTGGGCGACATCGCCGCCAGGGACCTGATCTGCGCCCAGAAGTTC | |
| | AACGGCCTGACCGTGCTGCCCCCCCTGCTGACCGACGAGATGATCGC | |
| | CCAGTACACCAGCGCCCTGCTGGCCGGCACCATCACCAGCGGCTGGA | |
| | CCTTCGGCGCCGGCCCCGCCCTGCAGATCCCCTTCCCCATGCAGATG | |
| | GCCTACAGGTTCAACGGCATCGGCGTGACCCAGAACGTGCTGTACGAG | |
| | AACCAGAAGCTGATCGCCAACCAGTTCAACAGCGCCATCGGCAAGATC | |
| | CAGGACAGCCTGAGCAGCACCCCCAGCGCCCTGGGCAAGCTGCAGGA | |
| | CGTGGTGAACCAGAACGCCCAGGCCCTGAACACCCTGGTGAAGCAGC | |
| | TGAGCAGCAACTTCGGCGCCATCAGCAGCGTGCTGAACGACATCCTGA | |
| | GCAGGCTGGACCCCCCCGAGGCCGAGGTGCAGATCGACAGGCTGATC | |
| | ACCGGCAGGCTGCAGAGCCTGCAGACCTACGTGACCCAGCAGCTGAT | |
| | CAGGGCCGCCGAGATCAGGGCCAGCGCCAACCTGGCCGCCACCAAGA | |
| | TGAGCGAGTGCGTGCTGGGCCAGAGCAAGAGGGTGGACTTCTGCGGC | |
| | AAGGGCTACCACCTGATGAGCTTCCCCCAGAGCGCCCCCCACGGCGT | |
| | GGTGTTCCTGCACGTGACCTACGTGCCCGCCCAGGAGAAGAACTTCAC | |
| | CACCGCCCCCGCCATCTGCCACGACGGCAAGGCCCACTTCCCCAGGG | |
| | AGGGCGTGTTCGTGAGCAACGGCACCCACTGGTTCGTGACCCAGAGG | |
| | AACTTCTACGAGCCCCAGATCATCACCACCGACAACACCTTCGTGAGC | |
| | GGCAACTGCGACGTGGTGATCGGCATCGTGAACAACACCGTGTACGAC | |
| | CCCCTGCAGCCCGAGCTGGACAGCTTCAAGGAGGAGCTGGACAAGTA | |
| | CTTCAAGAACCACACCAGCCCCGACGTGGACCTGGGCGACATCAGCG | |
| | GCATCAACGCCAGCGTGGTGAACATCCAGAAGGAGATCGACAGGCTGA | |
| | ACGAGGTGGCCAAGAACCTGAACGAGAGCCTGATCGACCTGCAGGAG | |
| | CTGGGCAAGTACGAGCAGTACATCAAGTGGCCCTGGTACATCTGGCTG | |
| | GGCTTCATCGCCGGCCTGATCGCCATCGTGATGGTGACCATCATGCTG | |
| | TGCTGCATGACCAGCTGCTGCAGCTGCCTGAAGGGCTGCTGCAGCTGC | |
| | GGCAGCTGCTGCAAGTTCGACGAGGACGACAGCGAGCCCGTGCTGAA | |
| | GGGCGTGAAGCTGCACTACACCGGAAGCGGAGCCACGAACTTCTCTCT | |
| | GTTAAAGCAAGCAGGAGATGTTGAAGAAAACCCCGGGCCTAGCGCCGT | |
| | GAAGAGGACCATCAAGGGCACCCACCACTGGCTGCTGCTGACCATCCT | |
| | GACCAGCCTGCTGGTGCTGGTGCAGAGCACCCAGTGGAGCCTGTTCTT | |
| | CTTCCTGTACGAGAACGCCTTCCTGCCCTTCGCCATGGGCATCATCGC | |
| | CATGAGCGCCTTCGCCATGATGTTCGTGAAGCACAAGCACGCCTTCCT | |
| | GTGCCTGTTCCTGCTGCCCAGCCTGGCCACCGTGGCCTACTTCAACAT | |
| | GGTGTACATGCCCGCCAGCTGGGTGATGAGGATCATGACCTGGCTGGA | |
| | CATGGTGGACACCAGCCTGAGCGGCTTCAAGCTGAAGGACTGCGTGAT | |
| | GTACGCCAGCGCCGTGGTGCTGCTGATCCTGATGACCGCCAGGACCG | |
| | TGTACGACGACGGCGCCAGGAGGGTGTGGACCCTGATGAACGTGCTG | |
| | ACCCTGGTGTACAAGGTGTACTACGGCAACGCCCTGGACCAGGCCATC | |
| | AGCATGTGGGCCCTGATCATCAGCGTGACCAGCAACTACAGCGGCGTG | |
| | GTGACCACCGTGATGTTCCTGGCCAGGGGCATCGTGTTCATGTGCGTG | |
| | GAGTACTGCCCCATCTTCTTCATCACCGGCAACACCCTGCAGTGCATCA | |
| | TGCTGGTGTACTGCTTCCTGGGCTACTTCTGCACCTGCTACTTCGGCCT | |
| | GTTCTGCCTGCTGAACAGGTACTTCAGGCTGACCCTGGGCGTGTACGA | |
| | CTACCTGGTGAGCACCCAGGAGTTCAGGTACATGAACAGCCAGGGCCT | |
| | GCTGCCCCCCAAGAACAGCATCGACGCCTTCAAGCTGAACATCAAGCT | |
| | GCTGGGCGTGGGCGGCAAGCCCTGCATCAAGGTGGCCACCGTGCAGG | |
| | GAAGCGGAGAGGGCAGGGGAAGTCTTCTAACATGCGGGGACGTGGAG | |
| | GAAAATCCCGGCCCCAGCAAGATGAGCGACGTGAAGTGCACCAGCGT | |
| | GGTGCTGCTGAGCGTGCTGCAGCAGCTGAGGGTGGAGAGCAGCAGCA | |
| | AGCTGTGGGCCCAGTGCGTGCAGCTGCACAACGACATCCTGCTGGCC | |
| | AAGGACACCACCGAGGCCTTCGAGAAGATGGTGAGCCTGCTGAGCGT | |
| | GCTGCTGAGCATGCAGGGCGCCGTGGACATCAACAAGCTGTGCGAGG | |
| | AGATGCTGGACAACAGGGCCACCCTGCAGGGAAGCGGACAGTGTACT | |
| | AATTATGCTCTCTTGAAATTGGCTGGAGATGTTGAGAGCAACCCAGGTC | |
| | CCGCCATCGCCAGCGAGTTCAGCAGCCTGCCCAGCTACGCCGCCTTC | |
| | GCCACCGCCCAGGAGGCCTACGAGCAGGCCGTGGCCAACGGCGACA | |
| | GCGAGGTGGTGCTGAAGAAGCTGAAGAAGAGCCTGAACGTGGCCAAG | |
| | AGCGAGTTCGACAGGGACGCCGCCATGCAGAGGAAGCTGGAGAAGAT | |
| | GGCCGACCAGGCCATGACCCAGATGTACAAGCAGGCCAGGAGCGAGG | |
| | ACAAGAGGGCCAAGGTGACCAGCGCCATGCAGACCATGCTGTTCACCA | |
| | TGCTGAGGAAGCTGGACAACGACGCCCTGAACAACATCATCAACAACG | |
| | CCAGGGACGGCTGCGTGCCCCTGAACATCATCCCCCTGACCACCGCC | |
| | GCCAAGCTGATGGTGGTGATCCCCGACTACAACACCTACAAGAACACC | |
| | TGCGACGGCACCACCTTCACCTACGCCAGCGCCCTGTGGGAGATCCA | |
| | GCAGGTGGTGGACGCCGACAGCAAGATCGTGCAGCTGAGCGAGATCA | |
| | GCATGGACAACAGCCCCAACCTGGCCTGGCCCCTGATCGTGACCGCC | |
| | CTGAGGGCCAACAGCGCCGTGAAGCTGCAGTGATGACTCGAGCTGGT | |
| | ACTGCATGCACGCAATGCTAGCTGCCCCTTTCCCGTCCTGGGTACCCC | |
| | GAGTCTCCCCCGACCTCGGGTCCCAGGTATGCTCCCACCTCCACCTGC | |
| | CCCACTCACCACCTCTGCTAGTTCCAGACACCTCCCAAGCACGCAGCA | |
| | ATGCAGCTCAAAACGCTTAGCCTAGCCACACCCCCACGGGAAACAGCA | |
| | GTGATTAACCTTTAGCAATAAACGAAAGTTTAACTAAGCTATACTAACCC | |
| | CAGGGTTGGTCAATTTCGTGCCAGCCACACCCTGGAGCTAGCAAAAAA | |
| | AA | |
| 14 | TAGTTATTAATAGTAATCAATTACGGGGTCATTAGTTCATAGCCCATATA | 299 |
| Ad5-Pan-CoV | TGGAGTTCCGCGTTACATAACTTACGGTAAATGGCCCGCCTGGCTGAC | |
| Spike-NSP9- | CGCCCAACGACCCCCGCCCATTGACGTCAATAATGACGTATGTTCCCAT | |
| NSP10- | AGTAACGCCAATAGGGACTTTCCATTGACGTCAATGGGTGGAGTATTTA | |
| NSP13 | CGGTAAACTGCCCACTTGGCAGTACATCAAGTGTATCATATGCCAAGTA | |
| | CGCCCCCTATTGACGTCAATGACGGTAAATGGCCCGCCTGGCATTATG | |
| | CCCAGTACATGACCTTATGGGACTTTCCTACTTGGCAGTACATCTACGT | |
| | ATTAGTCATCGCTATTACCATGGTGATGCGGTTTTGGCAGTACATCAAT | |
| | GGGCGTGGATAGCGGTTTGACTCACGGGGATTTCCAAGTCTCCACCCC | |
| | ATTGACGTCAATGGGAGTTTGTTTTGGCACCAAAATCAACGGGACTTTC | |
| | CAAAATGTCGTAACAACTCCGCCCCATTGACGCAAATGGGCGGTAGGC | |
| | GTGTACGGTGGGAGGTCTATATAAGCAGAGCTGGTTTAGTGAACCGTC | |
| | AGATCGAGAATAAACTAGTATTCTTCTGGTCCCCACAGACTCAGAGAGA | |
| | ACCCGCCACCATGTTCGTGTTCCTGGTGCTGCTGCCCCTGGTGAGCAG | |
| | CCAGTGCGTGAACCTGACCACCAGGACCCAGCTGCCCCCCGCCTACA | |
| | CCAACAGCTTCACCAGGGGCGTGTACTACCCCGACAAGGTGTTCAGGA | |
| | GCAGCGTGCTGCACAGCACCCAGGACCTGTTCCTGCCCTTCTTCAGCA | |
| | ACGTGACCTGGTTCCACGCCATCCACGTGAGCGGCACCAACGGCACCA | |
| | AGAGGTTCGACAACCCCGTGCTGCCCTTCAACGACGGCGTGTACTTCG | |
| | CCAGCACCGAGAAGAGCAACATCATCAGGGGCTGGATCTTCGGCACCA | |
| | CCCTGGACAGCAAGACCCAGAGCCTGCTGATCGTGAACAACGCCACCA | |
| | ACGTGGTGATCAAGGTGTGCGAGTTCCAGTTCTGCAACGACCCCTTCC | |
| | TGGGCGTGTACTACCACAAGAACAACAAGAGCTGGATGGAGAGCGAGT | |
| | TCAGGGTGTACAGCAGCGCCAACAACTGCACCTTCGAGTACGTGAGCC | |
| | AGCCCTTCCTGATGGACCTGGAGGGCAAGCAGGGCAACTTCAAGAACC | |
| | TGAGGGAGTTCGTGTTCAAGAACATCGACGGCTACTTCAAGATCTACAG | |
| | CAAGCACACCCCCATCAACCTGGTGAGGGACCTGCCCCAGGGCTTCAG | |
| | CGCCCTGGAGCCCCTGGTGGACCTGCCCATCGGCATCAACATCACCAG | |
| | GTTCCAGACCCTGCTGGCCCTGCACAGGAGCTACCTGACCCCCGGCG | |
| | ACAGCAGCAGCGGCTGGACCGCCGGCGCCGCCGCCTACTACGTGGGC | |
| | TACCTGCAGCCCAGGACCTTCCTGCTGAAGTACAACGAGAACGGCACC | |
| | ATCACCGACGCCGTGGACTGCGCCCTGGACCCCCTGAGCGAGACCAA | |
| | GTGCACCCTGAAGAGCTTCACCGTGGAGAAGGGCATCTACCAGACCAG | |
| | CAACTTCAGGGTGCAGCCCACCGAGAGCATCGTGAGGTTCCCCAACAT | |
| | CACCAACCTGTGCCCCTTCGGCGAGGTGTTCAACGCCACCAGGTTCGC | |
| | CAGCGTGTACGCCTGGAACAGGAAGAGGATCAGCAACTGCGTGGCCG | |
| | ACTACAGCGTGCTGTACAACAGCGCCAGCTTCAGCACCTTCAAGTGCT | |
| | ACGGCGTGAGCCCCACCAAGCTGAACGACCTGTGCTTCACCAACGTGT | |
| | ACGCCGACAGCTTCGTGATCAGGGGCGACGAGGTGAGGCAGATCGCC | |
| | CCCGGCCAGACCGGCAAGATCGCCGACTACAACTACAAGCTGCCCGA | |
| | CGACTTCACCGGCTGCGTGATCGCCTGGAACAGCAACAACCTGGACAG | |
| | CAAGGTGGGCGGCAACTACAACTACCTGTACAGGCTGTTCAGGAAGAG | |
| | CAACCTGAAGCCCTTCGAGAGGGACATCAGCACCGAGATCTACCAGGC | |
| | CGGCAGCACCCCCTGCAACGGCGTGGAGGGCTTCAACTGCTACTTCCC | |
| | CCTGCAGAGCTACGGCTTCCAGCCCACCAACGGCGTGGGCTACCAGC | |
| | CCTACAGGGTGGTGGTGCTGAGCTTCGAGCTGCTGCACGCCCCCGCC | |
| | ACCGTGTGCGGCCCCAAGAAGAGCACCAACCTGGTGAAGAACAAGTGC | |
| | GTGAACTTCAACTTCAACGGCCTGACCGGCACCGGCGTGCTGACCGAG | |
| | AGCAACAAGAAGTTCCTGCCCTTCCAGCAGTTCGGCAGGGACATCGCC | |
| | GACACCACCGACGCCGTGAGGGACCCCCAGACCCTGGAGATCCTGGA | |
| | CATCACCCCCTGCAGCTTCGGCGGCGTGAGCGTGATCACCCCCGGCA | |
| | CCAACACCAGCAACCAGGTGGCCGTGCTGTACCAGGACGTGAACTGCA | |
| | CCGAGGTGCCCGTGGCCATCCACGCCGACCAGCTGACCCCCACCTGG | |
| | AGGGTGTACAGCACCGGCAGCAACGTGTTCCAGACCAGGGCCGGCTG | |
| | CCTGATCGGCGCCGAGCACGTGAACAACAGCTACGAGTGCGACATCCC | |
| | CATCGGCGCCGGCATCTGCGCCAGCTACCAGACCCAGACCAACAGCC | |
| | CCAGGAGGGCCAGGAGCGTGGCCAGCCAGAGCATCATCGCCTACACC | |
| | ATGAGCCTGGGCGCCGAGAACAGCGTGGCCTACAGCAACAACAGCAT | |
| | CGCCATCCCCACCAACTTCACCATCAGCGTGACCACCGAGATCCTGCC | |
| | CGTGAGCATGACCAAGACCAGCGTGGACTGCACCATGTACATCTGCGG | |
| | CGACAGCACCGAGTGCAGCAACCTGCTGCTGCAGTACGGCAGCTTCTG | |
| | CACCCAGCTGAACAGGGCCCTGACCGGCATCGCCGTGGAGCAGGACA | |
| | AGAACACCCAGGAGGTGTTCGCCCAGGTGAAGCAGATCTACAAGACCC | |
| | CCCCCATCAAGGACTTCGGCGGCTTCAACTTCAGCCAGATCCTGCCCG | |
| | ACCCCAGCAAGCCCAGCAAGAGGAGCTTCATCGAGGACCTGCTGTTCA | |
| | ACAAGGTGACCCTGGCCGACGCCGGCTTCATCAAGCAGTACGGCGACT | |
| | GCCTGGGCGACATCGCCGCCAGGGACCTGATCTGCGCCCAGAAGTTC | |
| | AACGGCCTGACCGTGCTGCCCCCCCTGCTGACCGACGAGATGATCGC | |
| | CCAGTACACCAGCGCCCTGCTGGCCGGCACCATCACCAGCGGCTGGA | |
| | CCTTCGGCGCCGGCGCCGCCCTGCAGATCCCCTTCGCCATGCAGATG | |
| | GCCTACAGGTTCAACGGCATCGGCGTGACCCAGAACGTGCTGTACGAG | |
| | AACCAGAAGCTGATCGCCAACCAGTTCAACAGCGCCATCGGCAAGATC | |
| | CAGGACAGCCTGAGCAGCACCGCCAGCGCCCTGGGCAAGCTGCAGGA | |
| | CGTGGTGAACCAGAACGCCCAGGCCCTGAACACCCTGGTGAAGCAGC | |
| | TGAGCAGCAACTTCGGCGCCATCAGCAGCGTGCTGAACGACATCCTGA | |
| | GCAGGCTGGACCCCCCCGAGGCCGAGGTGCAGATCGACAGGCTGATC | |
| | ACCGGCAGGCTGCAGAGCCTGCAGACCTACGTGACCCAGCAGCTGAT | |
| | CAGGGCCGCCGAGATCAGGGCCAGCGCCAACCTGGCCGCCACCAAGA | |
| | TGAGCGAGTGCGTGCTGGGCCAGAGCAAGAGGGTGGACTTCTGCGGC | |
| | AAGGGCTACCACCTGATGAGCTTCCCCCAGAGCGCCCCCCACGGCGT | |
| | GGTGTTCCTGCACGTGACCTACGTGCCCGCCCAGGAGAAGAACTTCAC | |
| | CACCGCCCCCGCCATCTGCCACGACGGCAAGGCCCACTTCCCCAGGG | |
| | AGGGCGTGTTCGTGAGCAACGGCACCCACTGGTTCGTGACCCAGAGG | |
| | AACTTCTACGAGCCCCAGATCATCACCACCGACAACACCTTCGTGAGC | |
| | GGCAACTGCGACGTGGTGATCGGCATCGTGAACAACACCGTGTACGAC | |
| | CCCCTGCAGCCCGAGCTGGACAGCTTCAAGGAGGAGCTGGACAAGTA | |
| | CTTCAAGAACCACACCAGCCCCGACGTGGACCTGGGCGACATCAGCG | |
| | GCATCAACGCCAGCGTGGTGAACATCCAGAAGGAGATCGACAGGCTGA | |
| | ACGAGGTGGCCAAGAACCTGAACGAGAGCCTGATCGACCTGCAGGAG | |
| | CTGGGCAAGTACGAGCAGTACATCAAGTGGCCCTGGTACATCTGGCTG | |
| | GGCTTCATCGCCGGCCTGATCGCCATCGTGATGGTGACCATCATGCTG | |
| | TGCTGCATGACCAGCTGCTGCAGCTGCCTGAAGGGCTGCTGCAGCTGC | |
| | GGCAGCTGCTGCAAGTTCGACGAGGACGACAGCGAGCCCGTGCTGAA | |
| | GGGCGTGAAGCTGCACTACACCGGAAGCGGAGCCACGAACTTCTCTCT | |
| | GTTAAAGCAAGCAGGAGATGTTGAAGAAAACCCCGGGCCTAACAACGA | |
| | GCTGAGCCCCGTGGCCCTGAGGCAGATGAGCTGCGCCGCCGGCACCA | |
| | CCCAGACCGCCTGCACCGACGACAACGCCCTGGCCTACTACAACACCA | |
| | CCAAGGGCGGCAGGTTCGTGCTGGCCCTGCTGAGCGACCTGCAGGAC | |
| | CTGAAGTGGGCCAGGTTCCCCAAGAGCGACGGCACCGGCACCATCTA | |
| | CACCGAGCTGGAGCCCCCCTGCAGGTTCGTGACCGACACCCCCAAGG | |
| | GCCCCAAGGTGAAGTACCTGTACTTCATCAAGGGCCTGAACAACCTGA | |
| | ACAGGGGCATGGTGCTGGGCAGCCTGGCCGCCACCGTGAGGCTGCAG | |
| | GGAAGCGGAGAGGGCAGGGGAAGTCTTCTAACATGCGGGGACGTGGA | |
| | GGAAAATCCCGGCCCCGCCGGCAACGCCACCGAGGTGCCCGCCAACA | |
| | GCACCGTGCTGAGCTTCTGCGCCTTCGCCGTGGACGCCGCCAAGGCC | |
| | TACAAGGACTACCTGGCCAGCGGCGGCCAGCCCATCACCAACTGCGT | |
| | GAAGATGCTGTGCACCCACACCGGCACCGGCCAGGCCATCACCGTGA | |
| | CCCCCGAGGCCAACATGGACCAGGAGAGCTTCGGCGGCGCCAGCTGC | |
| | TGCCTGTACTGCAGGTGCCACATCGACCACCCCAACCCCAAGGGCTTC | |
| | TGCGACCTGAAGGGCAAGTACGTGCAGATCCCCACCACCTGCGCCAAC | |
| | GACCCCGTGGGCTTCACCCTGAAGAACACCGTGTGCACCGTGTGCGG | |
| | CATGTGGAAGGGCTACGGCTGCAGCTGCGACCAGCTGAGGGAGCCCA | |
| | TGCTGCAGGGAAGCGGACAGTGTACTAATTATGCTCTCTTGAAATTGGC | |
| | TGGAGATGTTGAGAGCAACCCAGGTCCCGCCGTGGGCGCCTGCGTGC | |
| | TGTGCAACAGCCAGACCAGCCTGAGGTGCGGCGCCTGCATCAGGAGG | |
| | CCCTTCCTGTGCTGCAAGTGCTGCTACGACCACGTGATCAGCACCAGC | |
| | CACAAGCTGGTGCTGAGCGTGAACCCCTACGTGTGCAACGCCCCCGG | |
| | CTGCGACGTGACCGACGTGACCCAGCTGTACCTGGGCGGCATGAGCT | |
| | ACTACTGCAAGAGCCACAAGCCCCCCATCAGCTTCCCCCTGTGCGCCA | |
| | ACGGCCAGGTGTTCGGCCTGTACAAGAACACCTGCGTGGGCAGCGAC | |
| | AACGTGACCGACTTCAACGCCATCGCCACCTGCGACTGGACCAACGCC | |
| | GGCGACTACATCCTGGCCAACACCTGCACCGAGAGGCTGAAGCTGTTC | |
| | GCCGCCGAGACCCTGAAGGCCACCGAGGAGACCTTCAAGCTGAGCTA | |
| | CGGCATCGCCACCGTGAGGGAGGTGCTGAGCGACAGGGAGCTGCACC | |
| | TGAGCTGGGAGGTGGGCAAGCCCAGGCCCCCCCTGAACAGGAACTAC | |
| | GTGTTCACCGGCTACAGGGTGACCAAGAACAGCAAGGTGCAGATCGGC | |
| | GAGTACACCTTCGAGAAGGGCGACTACGGCGACGCCGTGGTGTACAG | |
| | GGGCACCACCACCTACAAGCTGAACGTGGGCGACTACTTCGTGCTGAC | |
| | CAGCCACACCGTGATGCCCCTGAGCGCCCCCACCCTGGTGCCCCAGG | |
| | AGCACTACGTGAGGATCACCGGCCTGTACCCCACCCTGAACATCAGCG | |
| | ACGAGTTCAGCAGCAACGTGGCCAACTACCAGAAGGTGGGCATGCAGA | |
| | AGTACAGCACCCTGCAGGGCCCCCCCGGCACCGGCAAGAGCCACTTC | |
| | GCCATCGGCCTGGCCCTGTACTACCCCAGCGCCAGGATCGTGTACACC | |
| | GCCTGCAGCCACGCCGCCGTGGACGCCCTGTGCGAGAAGGCCCTGAA | |
| | GTACCTGCCCATCGACAAGTGCAGCAGGATCATCCCCGCCAGGGCCA | |
| | GGGTGGAGTGCTTCGACAAGTTCAAGGTGAACAGCACCCTGGAGCAGT | |
| | ACGTGTTCTGCACCGTGAACGCCCTGCCCGAGACCACCGCCGACATCG | |
| | TGGTGTTCGACGAGATCAGCATGGCCACCAACTACGACCTGAGCGTGG | |
| | TGAACGCCAGGCTGAGGGCCAAGCACTACGTGTACATCGGCGACCCC | |
| | GCCCAGCTGCCCGCCCCCAGGACCCTGCTGACCAAGGGCACCCTGGA | |
| | GCCCGAGTACTTCAACAGCGTGTGCAGGCTGATGAAGACCATCGGCCC | |
| | CGACATGTTCCTGGGCACCTGCAGGAGGTGCCCCGCCGAGATCGTGG | |
| | ACACCGTGAGCGCCCTGGTGTACGACAACAAGCTGAAGGCCCACAAG | |
| | GACAAGAGCGCCCAGTGCTTCAAGATGTTCTACAAGGGCGTGATCACC | |
| | CACGACGTGAGCAGCGCCATCAACAGGCCCCAGATCGGCGTGGTGAG | |
| | GGAGTTCCTGACCAGGAACCCCGCCTGGAGGAAGGCCGTGTTCATCA | |
| | GCCCCTACAACAGCCAGAACGCCGTGGCCAGCAAGATCCTGGGCCTG | |
| | CCCACCCAGACCGTGGACAGCAGCCAGGGCAGCGAGTACGACTACGT | |
| | GATCTTCACCCAGACCACCGAGACCGCCCACAGCTGCAACGTGAACAG | |
| | GTTCAACGTGGCCATCACCAGGGCCAAGGTGGGCATCCTGTGCATCAT | |
| | GAGCGACAGGGACCTGTACGACAAGCTGCAGTTCACCAGCCTGGAGAT | |
| | CCCCAGGAGGAACGTGGCCACCCTGCAGTGATGACTCGAGCTGGTACT | |
| | GCATGCACGCAATGCTAGCTGCCCCTTTCCCGTCCTGGGTACCCCGAG | |
| | TCTCCCCCGACCTCGGGTCCCAGGTATGCTCCCACCTCCACCTGCCCC | |
| | ACTCACCACCTCTGCTAGTTCCAGACACCTCCCAAGCACGCAGCAATG | |
| | CAGCTCAAAACGCTTAGCCTAGCCACACCCCCACGGGAAACAGCAGTG | |
| | ATTAACCTTTAGCAATAAACGAAAGTTTAACTAAGCTATACTAACCCCAG | |
| | GGTTGGTCAATTTCGTGCCAGCCACACCCTGGAGCTAGCAAAAAAAA | |
| 15 | TAGTTATTAATAGTAATCAATTACGGGGTCATTAGTTCATAGCCCATATA | 300 |
| Ad5-Pan-CoV | TGGAGTTCCGCGTTACATAACTTACGGTAAATGGCCCGCCTGGCTGAC | |
| HexaPro- | CGCCCAACGACCCCCGCCCATTGACGTCAATAATGACGTATGTTCCCAT | |
| NSP9-NSP10- | AGTAACGCCAATAGGGACTTTCCATTGACGTCAATGGGTGGAGTATTTA | |
| NSP13 | CGGTAAACTGCCCACTTGGCAGTACATCAAGTGTATCATATGCCAAGTA | |
| | CGCCCCCTATTGACGTCAATGACGGTAAATGGCCCGCCTGGCATTATG | |
| | CCCAGTACATGACCTTATGGGACTTTCCTACTTGGCAGTACATCTACGT | |
| | ATTAGTCATCGCTATTACCATGGTGATGCGGTTTTGGCAGTACATCAAT | |
| | GGGCGTGGATAGCGGTTTGACTCACGGGGATTTCCAAGTCTCCACCCC | |
| | ATTGACGTCAATGGGAGTTTGTTTTGGCACCAAAATCAACGGGACTTTC | |
| | CAAAATGTCGTAACAACTCCGCCCCATTGACGCAAATGGGCGGTAGGC | |
| | GTGTACGGTGGGAGGTCTATATAAGCAGAGCTGGTTTAGTGAACCGTC | |
| | AGATCGAGAATAAACTAGTATTCTTCTGGTCCCCACAGACTCAGAGAGA | |
| | ACCCGCCACCATGTTCGTGTTCCTGGTGCTGCTGCCCCTGGTGAGCAG | |
| | CCAGTGCGTGAACCTGACCACCAGGACCCAGCTGCCCCCCGCCTACA | |
| | CCAACAGCTTCACCAGGGGCGTGTACTACCCCGACAAGGTGTTCAGGA | |
| | GCAGCGTGCTGCACAGCACCCAGGACCTGTTCCTGCCCTTCTTCAGCA | |
| | ACGTGACCTGGTTCCACGCCATCCACGTGAGCGGCACCAACGGCACCA | |
| | AGAGGTTCGACAACCCCGTGCTGCCCTTCAACGACGGCGTGTACTTCG | |
| | CCAGCACCGAGAAGAGCAACATCATCAGGGGCTGGATCTTCGGCACCA | |
| | CCCTGGACAGCAAGACCCAGAGCCTGCTGATCGTGAACAACGCCACCA | |
| | ACGTGGTGATCAAGGTGTGCGAGTTCCAGTTCTGCAACGACCCCTTCC | |
| | TGGGCGTGTACTACCACAAGAACAACAAGAGCTGGATGGAGAGCGAGT | |
| | TCAGGGTGTACAGCAGCGCCAACAACTGCACCTTCGAGTACGTGAGCC | |
| | AGCCCTTCCTGATGGACCTGGAGGGCAAGCAGGGCAACTTCAAGAACC | |
| | TGAGGGAGTTCGTGTTCAAGAACATCGACGGCTACTTCAAGATCTACAG | |
| | CAAGCACACCCCCATCAACCTGGTGAGGGACCTGCCCCAGGGCTTCAG | |
| | CGCCCTGGAGCCCCTGGTGGACCTGCCCATCGGCATCAACATCACCAG | |
| | GTTCCAGACCCTGCTGGCCCTGCACAGGAGCTACCTGACCCCCGGCG | |
| | ACAGCAGCAGCGGCTGGACCGCCGGCGCCGCCGCCTACTACGTGGGC | |
| | TACCTGCAGCCCAGGACCTTCCTGCTGAAGTACAACGAGAACGGCACC | |
| | ATCACCGACGCCGTGGACTGCGCCCTGGACCCCCTGAGCGAGACCAA | |
| | GTGCACCCTGAAGAGCTTCACCGTGGAGAAGGGCATCTACCAGACCAG | |
| | CAACTTCAGGGTGCAGCCCACCGAGAGCATCGTGAGGTTCCCCAACAT | |
| | CACCAACCTGTGCCCCTTCGGCGAGGTGTTCAACGCCACCAGGTTCGC | |
| | CAGCGTGTACGCCTGGAACAGGAAGAGGATCAGCAACTGCGTGGCCG | |
| | ACTACAGCGTGCTGTACAACAGCGCCAGCTTCAGCACCTTCAAGTGCT | |
| | ACGGCGTGAGCCCCACCAAGCTGAACGACCTGTGCTTCACCAACGTGT | |
| | ACGCCGACAGCTTCGTGATCAGGGGCGACGAGGTGAGGCAGATCGCC | |
| | CCCGGCCAGACCGGCAAGATCGCCGACTACAACTACAAGCTGCCCGA | |
| | CGACTTCACCGGCTGCGTGATCGCCTGGAACAGCAACAACCTGGACAG | |
| | CAAGGTGGGCGGCAACTACAACTACCTGTACAGGCTGTTCAGGAAGAG | |
| | CAACCTGAAGCCCTTCGAGAGGGACATCAGCACCGAGATCTACCAGGC | |
| | CGGCAGCACCCCCTGCAACGGCGTGGAGGGCTTCAACTGCTACTTCCC | |
| | CCTGCAGAGCTACGGCTTCCAGCCCACCAACGGCGTGGGCTACCAGC | |
| | CCTACAGGGTGGTGGTGCTGAGCTTCGAGCTGCTGCACGCCCCCGCC | |
| | ACCGTGTGCGGCCCCAAGAAGAGCACCAACCTGGTGAAGAACAAGTGC | |
| | GTGAACTTCAACTTCAACGGCCTGACCGGCACCGGCGTGCTGACCGAG | |
| | AGCAACAAGAAGTTCCTGCCCTTCCAGCAGTTCGGCAGGGACATCGCC | |
| | GACACCACCGACGCCGTGAGGGACCCCCAGACCCTGGAGATCCTGGA | |
| | CATCACCCCCTGCAGCTTCGGCGGCGTGAGCGTGATCACCCCCGGCA | |
| | CCAACACCAGCAACCAGGTGGCCGTGCTGTACCAGGACGTGAACTGCA | |
| | CCGAGGTGCCCGTGGCCATCCACGCCGACCAGCTGACCCCCACCTGG | |
| | AGGGTGTACAGCACCGGCAGCAACGTGTTCCAGACCAGGGCCGGCTG | |
| | CCTGATCGGCGCCGAGCACGTGAACAACAGCTACGAGTGCGACATCCC | |
| | CATCGGCGCCGGCATCTGCGCCAGCTACCAGACCCAGACCAACAGCC | |
| | CCGGCAGCGCCAGCAGCGTGGCCAGCCAGAGCATCATCGCCTACACC | |
| | ATGAGCCTGGGCGCCGAGAACAGCGTGGCCTACAGCAACAACAGCAT | |
| | CGCCATCCCCACCAACTTCACCATCAGCGTGACCACCGAGATCCTGCC | |
| | CGTGAGCATGACCAAGACCAGCGTGGACTGCACCATGTACATCTGCGG | |
| | CGACAGCACCGAGTGCAGCAACCTGCTGCTGCAGTACGGCAGCTTCTG | |
| | CACCCAGCTGAACAGGGCCCTGACCGGCATCGCCGTGGAGCAGGACA | |
| | AGAACACCCAGGAGGTGTTCGCCCAGGTGAAGCAGATCTACAAGACCC | |
| | CCCCCATCAAGGACTTCGGCGGCTTCAACTTCAGCCAGATCCTGCCCG | |
| | ACCCCAGCAAGCCCAGCAAGAGGAGCCCCATCGAGGACCTGCTGTTCA | |
| | ACAAGGTGACCCTGGCCGACGCCGGCTTCATCAAGCAGTACGGCGACT | |
| | GCCTGGGCGACATCGCCGCCAGGGACCTGATCTGCGCCCAGAAGTTC | |
| | AACGGCCTGACCGTGCTGCCCCCCCTGCTGACCGACGAGATGATCGC | |
| | CCAGTACACCAGCGCCCTGCTGGCCGGCACCATCACCAGCGGCTGGA | |
| | CCTTCGGCGCCGGCCCCGCCCTGCAGATCCCCTTCCCCATGCAGATG | |
| | GCCTACAGGTTCAACGGCATCGGCGTGACCCAGAACGTGCTGTACGAG | |
| | AACCAGAAGCTGATCGCCAACCAGTTCAACAGCGCCATCGGCAAGATC | |
| | CAGGACAGCCTGAGCAGCACCCCCAGCGCCCTGGGCAAGCTGCAGGA | |
| | CGTGGTGAACCAGAACGCCCAGGCCCTGAACACCCTGGTGAAGCAGC | |
| | TGAGCAGCAACTTCGGCGCCATCAGCAGCGTGCTGAACGACATCCTGA | |
| | GCAGGCTGGACCCCCCCGAGGCCGAGGTGCAGATCGACAGGCTGATC | |
| | ACCGGCAGGCTGCAGAGCCTGCAGACCTACGTGACCCAGCAGCTGAT | |
| | CAGGGCCGCCGAGATCAGGGCCAGCGCCAACCTGGCCGCCACCAAGA | |
| | TGAGCGAGTGCGTGCTGGGCCAGAGCAAGAGGGTGGACTTCTGCGGC | |
| | AAGGGCTACCACCTGATGAGCTTCCCCCAGAGCGCCCCCCACGGCGT | |
| | GGTGTTCCTGCACGTGACCTACGTGCCCGCCCAGGAGAAGAACTTCAC | |
| | CACCGCCCCCGCCATCTGCCACGACGGCAAGGCCCACTTCCCCAGGG | |
| | AGGGCGTGTTCGTGAGCAACGGCACCCACTGGTTCGTGACCCAGAGG | |
| | AACTTCTACGAGCCCCAGATCATCACCACCGACAACACCTTCGTGAGC | |
| | GGCAACTGCGACGTGGTGATCGGCATCGTGAACAACACCGTGTACGAC | |
| | CCCCTGCAGCCCGAGCTGGACAGCTTCAAGGAGGAGCTGGACAAGTA | |
| | CTTCAAGAACCACACCAGCCCCGACGTGGACCTGGGCGACATCAGCG | |
| | GCATCAACGCCAGCGTGGTGAACATCCAGAAGGAGATCGACAGGCTGA | |
| | ACGAGGTGGCCAAGAACCTGAACGAGAGCCTGATCGACCTGCAGGAG | |
| | CTGGGCAAGTACGAGCAGTACATCAAGTGGCCCTGGTACATCTGGCTG | |
| | GGCTTCATCGCCGGCCTGATCGCCATCGTGATGGTGACCATCATGCTG | |
| | TGCTGCATGACCAGCTGCTGCAGCTGCCTGAAGGGCTGCTGCAGCTGC | |
| | GGCAGCTGCTGCAAGTTCGACGAGGACGACAGCGAGCCCGTGCTGAA | |
| | GGGCGTGAAGCTGCACTACACCGGAAGCGGAGCCACGAACTTCTCTCT | |
| | GTTAAAGCAAGCAGGAGATGTTGAAGAAAACCCCGGGCCTAACAACGA | |
| | GCTGAGCCCCGTGGCCCTGAGGCAGATGAGCTGCGCCGCCGGCACCA | |
| | CCCAGACCGCCTGCACCGACGACAACGCCCTGGCCTACTACAACACCA | |
| | CCAAGGGCGGCAGGTTCGTGCTGGCCCTGCTGAGCGACCTGCAGGAC | |
| | CTGAAGTGGGCCAGGTTCCCCAAGAGCGACGGCACCGGCACCATCTA | |
| | CACCGAGCTGGAGCCCCCCTGCAGGTTCGTGACCGACACCCCCAAGG | |
| | GCCCCAAGGTGAAGTACCTGTACTTCATCAAGGGCCTGAACAACCTGA | |
| | ACAGGGGCATGGTGCTGGGCAGCCTGGCCGCCACCGTGAGGCTGCAG | |
| | GGAAGCGGAGAGGGCAGGGGAAGTCTTCTAACATGCGGGGACGTGGA | |
| | GGAAAATCCCGGCCCCGCCGGCAACGCCACCGAGGTGCCCGCCAACA | |
| | GCACCGTGCTGAGCTTCTGCGCCTTCGCCGTGGACGCCGCCAAGGCC | |
| | TACAAGGACTACCTGGCCAGCGGCGGCCAGCCCATCACCAACTGCGT | |
| | GAAGATGCTGTGCACCCACACCGGCACCGGCCAGGCCATCACCGTGA | |
| | CCCCCGAGGCCAACATGGACCAGGAGAGCTTCGGCGGCGCCAGCTGC | |
| | TGCCTGTACTGCAGGTGCCACATCGACCACCCCAACCCCAAGGGCTTC | |
| | TGCGACCTGAAGGGCAAGTACGTGCAGATCCCCACCACCTGCGCCAAC | |
| | GACCCCGTGGGCTTCACCCTGAAGAACACCGTGTGCACCGTGTGCGG | |
| | CATGTGGAAGGGCTACGGCTGCAGCTGCGACCAGCTGAGGGAGCCCA | |
| | TGCTGCAGGGAAGCGGACAGTGTACTAATTATGCTCTCTTGAAATTGGC | |
| | TGGAGATGTTGAGAGCAACCCAGGTCCCGCCGTGGGCGCCTGCGTGC | |
| | TGTGCAACAGCCAGACCAGCCTGAGGTGCGGCGCCTGCATCAGGAGG | |
| | CCCTTCCTGTGCTGCAAGTGCTGCTACGACCACGTGATCAGCACCAGC | |
| | CACAAGCTGGTGCTGAGCGTGAACCCCTACGTGTGCAACGCCCCCGG | |
| | CTGCGACGTGACCGACGTGACCCAGCTGTACCTGGGCGGCATGAGCT | |
| | ACTACTGCAAGAGCCACAAGCCCCCCATCAGCTTCCCCCTGTGCGCCA | |
| | ACGGCCAGGTGTTCGGCCTGTACAAGAACACCTGCGTGGGCAGCGAC | |
| | AACGTGACCGACTTCAACGCCATCGCCACCTGCGACTGGACCAACGCC | |
| | GGCGACTACATCCTGGCCAACACCTGCACCGAGAGGCTGAAGCTGTTC | |
| | GCCGCCGAGACCCTGAAGGCCACCGAGGAGACCTTCAAGCTGAGCTA | |
| | CGGCATCGCCACCGTGAGGGAGGTGCTGAGCGACAGGGAGCTGCACC | |
| | TGAGCTGGGAGGTGGGCAAGCCCAGGCCCCCCCTGAACAGGAACTAC | |
| | GTGTTCACCGGCTACAGGGTGACCAAGAACAGCAAGGTGCAGATCGGC | |
| | GAGTACACCTTCGAGAAGGGCGACTACGGCGACGCCGTGGTGTACAG | |
| | GGGCACCACCACCTACAAGCTGAACGTGGGCGACTACTTCGTGCTGAC | |
| | CAGCCACACCGTGATGCCCCTGAGCGCCCCCACCCTGGTGCCCCAGG | |
| | AGCACTACGTGAGGATCACCGGCCTGTACCCCACCCTGAACATCAGCG | |
| | ACGAGTTCAGCAGCAACGTGGCCAACTACCAGAAGGTGGGCATGCAGA | |
| | AGTACAGCACCCTGCAGGGCCCCCCCGGCACCGGCAAGAGCCACTTC | |
| | GCCATCGGCCTGGCCCTGTACTACCCCAGCGCCAGGATCGTGTACACC | |
| | GCCTGCAGCCACGCCGCCGTGGACGCCCTGTGCGAGAAGGCCCTGAA | |
| | GTACCTGCCCATCGACAAGTGCAGCAGGATCATCCCCGCCAGGGCCA | |
| | GGGTGGAGTGCTTCGACAAGTTCAAGGTGAACAGCACCCTGGAGCAGT | |
| | ACGTGTTCTGCACCGTGAACGCCCTGCCCGAGACCACCGCCGACATCG | |
| | TGGTGTTCGACGAGATCAGCATGGCCACCAACTACGACCTGAGCGTGG | |
| | TGAACGCCAGGCTGAGGGCCAAGCACTACGTGTACATCGGCGACCCC | |
| | GCCCAGCTGCCCGCCCCCAGGACCCTGCTGACCAAGGGCACCCTGGA | |
| | GCCCGAGTACTTCAACAGCGTGTGCAGGCTGATGAAGACCATCGGCCC | |
| | CGACATGTTCCTGGGCACCTGCAGGAGGTGCCCCGCCGAGATCGTGG | |
| | ACACCGTGAGCGCCCTGGTGTACGACAACAAGCTGAAGGCCCACAAG | |
| | GACAAGAGCGCCCAGTGCTTCAAGATGTTCTACAAGGGCGTGATCACC | |
| | CACGACGTGAGCAGCGCCATCAACAGGCCCCAGATCGGCGTGGTGAG | |
| | GGAGTTCCTGACCAGGAACCCCGCCTGGAGGAAGGCCGTGTTCATCA | |
| | GCCCCTACAACAGCCAGAACGCCGTGGCCAGCAAGATCCTGGGCCTG | |
| | CCCACCCAGACCGTGGACAGCAGCCAGGGCAGCGAGTACGACTACGT | |
| | GATCTTCACCCAGACCACCGAGACCGCCCACAGCTGCAACGTGAACAG | |
| | GTTCAACGTGGCCATCACCAGGGCCAAGGTGGGCATCCTGTGCATCAT | |
| | GAGCGACAGGGACCTGTACGACAAGCTGCAGTTCACCAGCCTGGAGAT | |
| | CCCCAGGAGGAACGTGGCCACCCTGCAGTGATGACTCGAGCTGGTACT | |
| | GCATGCACGCAATGCTAGCTGCCCCTTTCCCGTCCTGGGTACCCCGAG | |
| | TCTCCCCCGACCTCGGGTCCCAGGTATGCTCCCACCTCCACCTGCCCC | |
| | ACTCACCACCTCTGCTAGTTCCAGACACCTCCCAAGCACGCAGCAATG | |
| | CAGCTCAAAACGCTTAGCCTAGCCACACCCCCACGGGAAACAGCAGTG | |
| | ATTAACCTTTAGCAATAAACGAAAGTTTAACTAAGCTATACTAACCCCAG | |
| | GGTTGGTCAATTTCGTGCCAGCCACACCCTGGAGCTAGCAAAAAAAA | |
| 16 | TAGTTATTAATAGTAATCAATTACGGGGTCATTAGTTCATAGCCCATATA | 301 |
| Ad5-Pan-CoV | TGGAGTTCCGCGTTACATAACTTACGGTAAATGGCCCGCCTGGCTGAC | |
| Spike-NSP14- | CGCCCAACGACCCCCGCCCATTGACGTCAATAATGACGTATGTTCCCAT | |
| NSP15 | AGTAACGCCAATAGGGACTTTCCATTGACGTCAATGGGTGGAGTATTTA | |
| | CGGTAAACTGCCCACTTGGCAGTACATCAAGTGTATCATATGCCAAGTA | |
| | CGCCCCCTATTGACGTCAATGACGGTAAATGGCCCGCCTGGCATTATG | |
| | CCCAGTACATGACCTTATGGGACTTTCCTACTTGGCAGTACATCTACGT | |
| | ATTAGTCATCGCTATTACCATGGTGATGCGGTTTTGGCAGTACATCAAT | |
| | GGGCGTGGATAGCGGTTTGACTCACGGGGATTTCCAAGTCTCCACCCC | |
| | ATTGACGTCAATGGGAGTTTGTTTTGGCACCAAAATCAACGGGACTTTC | |
| | CAAAATGTCGTAACAACTCCGCCCCATTGACGCAAATGGGCGGTAGGC | |
| | GTGTACGGTGGGAGGTCTATATAAGCAGAGCTGGTTTAGTGAACCGTC | |
| | AGATCGAGAATAAACTAGTATTCTTCTGGTCCCCACAGACTCAGAGAGA | |
| | ACCCGCCACCATGTTCGTGTTCCTGGTGCTGCTGCCCCTGGTGAGCAG | |
| | CCAGTGCGTGAACCTGACCACCAGGACCCAGCTGCCCCCCGCCTACA | |
| | CCAACAGCTTCACCAGGGGCGTGTACTACCCCGACAAGGTGTTCAGGA | |
| | GCAGCGTGCTGCACAGCACCCAGGACCTGTTCCTGCCCTTCTTCAGCA | |
| | ACGTGACCTGGTTCCACGCCATCCACGTGAGCGGCACCAACGGCACCA | |
| | AGAGGTTCGACAACCCCGTGCTGCCCTTCAACGACGGCGTGTACTTCG | |
| | CCAGCACCGAGAAGAGCAACATCATCAGGGGCTGGATCTTCGGCACCA | |
| | CCCTGGACAGCAAGACCCAGAGCCTGCTGATCGTGAACAACGCCACCA | |
| | ACGTGGTGATCAAGGTGTGCGAGTTCCAGTTCTGCAACGACCCCTTCC | |
| | TGGGCGTGTACTACCACAAGAACAACAAGAGCTGGATGGAGAGCGAGT | |
| | TCAGGGTGTACAGCAGCGCCAACAACTGCACCTTCGAGTACGTGAGCC | |
| | AGCCCTTCCTGATGGACCTGGAGGGCAAGCAGGGCAACTTCAAGAACC | |
| | TGAGGGAGTTCGTGTTCAAGAACATCGACGGCTACTTCAAGATCTACAG | |
| | CAAGCACACCCCCATCAACCTGGTGAGGGACCTGCCCCAGGGCTTCAG | |
| | CGCCCTGGAGCCCCTGGTGGACCTGCCCATCGGCATCAACATCACCAG | |
| | GTTCCAGACCCTGCTGGCCCTGCACAGGAGCTACCTGACCCCCGGCG | |
| | ACAGCAGCAGCGGCTGGACCGCCGGCGCCGCCGCCTACTACGTGGGC | |
| | TACCTGCAGCCCAGGACCTTCCTGCTGAAGTACAACGAGAACGGCACC | |
| | ATCACCGACGCCGTGGACTGCGCCCTGGACCCCCTGAGCGAGACCAA | |
| | GTGCACCCTGAAGAGCTTCACCGTGGAGAAGGGCATCTACCAGACCAG | |
| | CAACTTCAGGGTGCAGCCCACCGAGAGCATCGTGAGGTTCCCCAACAT | |
| | CACCAACCTGTGCCCCTTCGGCGAGGTGTTCAACGCCACCAGGTTCGC | |
| | CAGCGTGTACGCCTGGAACAGGAAGAGGATCAGCAACTGCGTGGCCG | |
| | ACTACAGCGTGCTGTACAACAGCGCCAGCTTCAGCACCTTCAAGTGCT | |
| | ACGGCGTGAGCCCCACCAAGCTGAACGACCTGTGCTTCACCAACGTGT | |
| | ACGCCGACAGCTTCGTGATCAGGGGCGACGAGGTGAGGCAGATCGCC | |
| | CCCGGCCAGACCGGCAAGATCGCCGACTACAACTACAAGCTGCCCGA | |
| | CGACTTCACCGGCTGCGTGATCGCCTGGAACAGCAACAACCTGGACAG | |
| | CAAGGTGGGCGGCAACTACAACTACCTGTACAGGCTGTTCAGGAAGAG | |
| | CAACCTGAAGCCCTTCGAGAGGGACATCAGCACCGAGATCTACCAGGC | |
| | CGGCAGCACCCCCTGCAACGGCGTGGAGGGCTTCAACTGCTACTTCCC | |
| | CCTGCAGAGCTACGGCTTCCAGCCCACCAACGGCGTGGGCTACCAGC | |
| | CCTACAGGGTGGTGGTGCTGAGCTTCGAGCTGCTGCACGCCCCCGCC | |
| | ACCGTGTGCGGCCCCAAGAAGAGCACCAACCTGGTGAAGAACAAGTGC | |
| | GTGAACTTCAACTTCAACGGCCTGACCGGCACCGGCGTGCTGACCGAG | |
| | AGCAACAAGAAGTTCCTGCCCTTCCAGCAGTTCGGCAGGGACATCGCC | |
| | GACACCACCGACGCCGTGAGGGACCCCCAGACCCTGGAGATCCTGGA | |
| | CATCACCCCCTGCAGCTTCGGCGGCGTGAGCGTGATCACCCCCGGCA | |
| | CCAACACCAGCAACCAGGTGGCCGTGCTGTACCAGGACGTGAACTGCA | |
| | CCGAGGTGCCCGTGGCCATCCACGCCGACCAGCTGACCCCCACCTGG | |
| | AGGGTGTACAGCACCGGCAGCAACGTGTTCCAGACCAGGGCCGGCTG | |
| | CCTGATCGGCGCCGAGCACGTGAACAACAGCTACGAGTGCGACATCCC | |
| | CATCGGCGCCGGCATCTGCGCCAGCTACCAGACCCAGACCAACAGCC | |
| | CCAGGAGGGCCAGGAGCGTGGCCAGCCAGAGCATCATCGCCTACACC | |
| | ATGAGCCTGGGCGCCGAGAACAGCGTGGCCTACAGCAACAACAGCAT | |
| | CGCCATCCCCACCAACTTCACCATCAGCGTGACCACCGAGATCCTGCC | |
| | CGTGAGCATGACCAAGACCAGCGTGGACTGCACCATGTACATCTGCGG | |
| | CGACAGCACCGAGTGCAGCAACCTGCTGCTGCAGTACGGCAGCTTCTG | |
| | CACCCAGCTGAACAGGGCCCTGACCGGCATCGCCGTGGAGCAGGACA | |
| | AGAACACCCAGGAGGTGTTCGCCCAGGTGAAGCAGATCTACAAGACCC | |
| | CCCCCATCAAGGACTTCGGCGGCTTCAACTTCAGCCAGATCCTGCCCG | |
| | ACCCCAGCAAGCCCAGCAAGAGGAGCTTCATCGAGGACCTGCTGTTCA | |
| | ACAAGGTGACCCTGGCCGACGCCGGCTTCATCAAGCAGTACGGCGACT | |
| | GCCTGGGCGACATCGCCGCCAGGGACCTGATCTGCGCCCAGAAGTTC | |
| | AACGGCCTGACCGTGCTGCCCCCCCTGCTGACCGACGAGATGATCGC | |
| | CCAGTACACCAGCGCCCTGCTGGCCGGCACCATCACCAGCGGCTGGA | |
| | CCTTCGGCGCCGGCGCCGCCCTGCAGATCCCCTTCGCCATGCAGATG | |
| | GCCTACAGGTTCAACGGCATCGGCGTGACCCAGAACGTGCTGTACGAG | |
| | AACCAGAAGCTGATCGCCAACCAGTTCAACAGCGCCATCGGCAAGATC | |
| | CAGGACAGCCTGAGCAGCACCGCCAGCGCCCTGGGCAAGCTGCAGGA | |
| | CGTGGTGAACCAGAACGCCCAGGCCCTGAACACCCTGGTGAAGCAGC | |
| | TGAGCAGCAACTTCGGCGCCATCAGCAGCGTGCTGAACGACATCCTGA | |
| | GCAGGCTGGACCCCCCCGAGGCCGAGGTGCAGATCGACAGGCTGATC | |
| | ACCGGCAGGCTGCAGAGCCTGCAGACCTACGTGACCCAGCAGCTGAT | |
| | CAGGGCCGCCGAGATCAGGGCCAGCGCCAACCTGGCCGCCACCAAGA | |
| | TGAGCGAGTGCGTGCTGGGCCAGAGCAAGAGGGTGGACTTCTGCGGC | |
| | AAGGGCTACCACCTGATGAGCTTCCCCCAGAGCGCCCCCCACGGCGT | |
| | GGTGTTCCTGCACGTGACCTACGTGCCCGCCCAGGAGAAGAACTTCAC | |
| | CACCGCCCCCGCCATCTGCCACGACGGCAAGGCCCACTTCCCCAGGG | |
| | AGGGCGTGTTCGTGAGCAACGGCACCCACTGGTTCGTGACCCAGAGG | |
| | AACTTCTACGAGCCCCAGATCATCACCACCGACAACACCTTCGTGAGC | |
| | GGCAACTGCGACGTGGTGATCGGCATCGTGAACAACACCGTGTACGAC | |
| | CCCCTGCAGCCCGAGCTGGACAGCTTCAAGGAGGAGCTGGACAAGTA | |
| | CTTCAAGAACCACACCAGCCCCGACGTGGACCTGGGCGACATCAGCG | |
| | GCATCAACGCCAGCGTGGTGAACATCCAGAAGGAGATCGACAGGCTGA | |
| | ACGAGGTGGCCAAGAACCTGAACGAGAGCCTGATCGACCTGCAGGAG | |
| | CTGGGCAAGTACGAGCAGTACATCAAGTGGCCCTGGTACATCTGGCTG | |
| | GGCTTCATCGCCGGCCTGATCGCCATCGTGATGGTGACCATCATGCTG | |
| | TGCTGCATGACCAGCTGCTGCAGCTGCCTGAAGGGCTGCTGCAGCTGC | |
| | GGCAGCTGCTGCAAGTTCGACGAGGACGACAGCGAGCCCGTGCTGAA | |
| | GGGCGTGAAGCTGCACTACACCGGAAGCGGAGCCACGAACTTCTCTCT | |
| | GTTAAAGCAAGCAGGAGATGTTGAAGAAAACCCCGGGCCTGCCGAGAA | |
| | CGTGACCGGCCTGTTCAAGGACTGCAGCAAGGTGATCACCGGCCTGCA | |
| | CCCCACCCAGGCCCCCACCCACCTGAGCGTGGACACCAAGTTCAAGAC | |
| | CGAGGGCCTGTGCGTGGACATCCCCGGCATCCCCAAGGACATGACCT | |
| | ACAGGAGGCTGATCAGCATGATGGGCTTCAAGATGAACTACCAGGTGA | |
| | ACGGCTACCCCAACATGTTCATCACCAGGGAGGAGGCCATCAGGCACG | |
| | TGAGGGCCTGGATCGGCTTCGACGTGGAGGGCTGCCACGCCACCAGG | |
| | GAGGCCGTGGGCACCAACCTGCCCCTGCAGCTGGGCTTCAGCACCGG | |
| | CGTGAACCTGGTGGCCGTGCCCACCGGCTACGTGGACACCCCCAACA | |
| | ACACCGACTTCAGCAGGGTGAGCGCCAAGCCCCCCCCCGGCGACCAG | |
| | TTCAAGCACCTGATCCCCCTGATGTACAAGGGCCTGCCCTGGAACGTG | |
| | GTGAGGATCAAGATCGTGCAGATGCTGAGCGACACCCTGAAGAACCTG | |
| | AGCGACAGGGTGGTGTTCGTGCTGTGGGCCCACGGCTTCGAGCTGAC | |
| | CAGCATGAAGTACTTCGTGAAGATCGGCCCCGAGAGGACCTGCTGCCT | |
| | GTGCGACAGGAGGGCCACCTGCTTCAGCACCGCCAGCGACACCTACG | |
| | CCTGCTGGCACCACAGCATCGGCTTCGACTACGTGTACAACCCCTTCA | |
| | TGATCGACGTGCAGCAGTGGGGCTTCACCGGCAACCTGCAGAGCAAC | |
| | CACGACCTGTACTGCCAGGTGCACGGCAACGCCCACGTGGCCAGCTG | |
| | CGACGCCATCATGACCAGGTGCCTGGCCGTGCACGAGTGCTTCGTGAA | |
| | GAGGGTGGACTGGACCATCGAGTACCCCATCATCGGCGACGAGCTGA | |
| | AGATCAACGCCGCCTGCAGGAAGGTGCAGCACATGGTGGTGAAGGCC | |
| | GCCCTGCTGGCCGACAAGTTCCCCGTGCTGCACGACATCGGCAACCC | |
| | CAAGGCCATCAAGTGCGTGCCCCAGGCCGACGTGGAGTGGAAGTTCTA | |
| | CGACGCCCAGCCCTGCAGCGACAAGGCCTACAAGATCGAGGAGCTGT | |
| | TCTACAGCTACGCCACCCACAGCGACAAGTTCACCGACGGCGTGTGCC | |
| | TGTTCTGGAACTGCAACGTGGACAGGTACCCCGCCAACAGCATCGTGT | |
| | GCAGGTTCGACACCAGGGTGCTGAGCAACCTGAACCTGCCCGGCTGC | |
| | GACGGCGGCAGCCTGTACGTGAACAAGCACGCCTTCCACACCCCCGC | |
| | CTTCGACAAGAGCGCCTTCGTGAACCTGAAGCAGCTGCCCTTCTTCTAC | |
| | TACAGCGACAGCCCCTGCGAGAGCCACGGCAAGCAGGTGGTGAGCGA | |
| | CATCGACTACGTGCCCCTGAAGAGCGCCACCTGCATCACCAGGTGCAA | |
| | CCTGGGCGGCGCCGTGTGCAGGCACCACGCCAACGAGTACAGGCTGT | |
| | ACCTGGACGCCTACAACATGATGATCAGCGCCGGCTTCAGCCTGTGGG | |
| | TGTACAAGCAGTTCGACACCTACAACCTGTGGAACACCTTCACCAGGCT | |
| | GCAGGGAAGCGGAGAGGGCAGGGGAAGTCTTCTAACATGCGGGGACG | |
| | TGGAGGAAAATCCCGGCCCCAGCCTGGAGAACGTGGCCTTCAACGTG | |
| | GTGAACAAGGGCCACTTCGACGGCCAGCAGGGCGAGGTGCCCGTGAG | |
| | CATCATCAACAACACCGTGTACACCAAGGTGGACGGCGTGGACGTGGA | |
| | GCTGTTCGAGAACAAGACCACCCTGCCCGTGAACGTGGCCTTCGAGCT | |
| | GTGGGCCAAGAGGAACATCAAGCCCGTGCCCGAGGTGAAGATCCTGA | |
| | ACAACCTGGGCGTGGACATCGCCGCCAACACCGTGATCTGGGACTACA | |
| | AGAGGGACGCCCCCGCCCACATCAGCACCATCGGCGTGTGCAGCATG | |
| | ACCGACATCGCCAAGAAGCCCACCGAGACCATCTGCGCCCCCCTGACC | |
| | GTGTTCTTCGACGGCAGGGTGGACGGCCAGGTGGACCTGTTCAGGAA | |
| | CGCCAGGAACGGCGTGCTGATCACCGAGGGCAGCGTGAAGGGCCTGC | |
| | AGCCCAGCGTGGGCCCCAAGCAGGCCAGCCTGAACGGCGTGACCCTG | |
| | ATCGGCGAGGCCGTGAAGACCCAGTTCAACTACTACAAGAAGGTGGAC | |
| | GGCGTGGTGCAGCAGCTGCCCGAGACCTACTTCACCCAGAGCAGGAA | |
| | CCTGCAGGAGTTCAAGCCCAGGAGCCAGATGGAGATCGACTTCCTGGA | |
| | GCTGGCCATGGACGAGTTCATCGAGAGGTACAAGCTGGAGGGCTACG | |
| | CCTTCGAGCACATCGTGTACGGCGACTTCAGCCACAGCCAGCTGGGCG | |
| | GCCTGCACCTGCTGATCGGCCTGGCCAAGAGGTTCAAGGAGAGCCCC | |
| | TTCGAGCTGGAGGACTTCATCCCCATGGACAGCACCGTGAAGAACTAC | |
| | TTCATCACCGACGCCCAGACCGGCAGCAGCAAGTGCGTGTGCAGCGT | |
| | GATCGACCTGCTGCTGGACGACTTCGTGGAGATCATCAAGAGCCAGGA | |
| | CCTGAGCGTGGTGAGCAAGGTGGTGAAGGTGACCATCGACTACACCGA | |
| | GATCAGCTTCATGCTGTGGTGCAAGGACGGCCACGTGGAGACCTTCTA | |
| | CCCCAAGCTGCAGTGATGACTCGAGCTGGTACTGCATGCACGCAATGC | |
| | TAGCTGCCCCTTTCCCGTCCTGGGTACCCCGAGTCTCCCCCGACCTCG | |
| | GGTCCCAGGTATGCTCCCACCTCCACCTGCCCCACTCACCACCTCTGC | |
| | TAGTTCCAGACACCTCCCAAGCACGCAGCAATGCAGCTCAAAACGCTTA | |
| | GCCTAGCCACACCCCCACGGGAAACAGCAGTGATTAACCTTTAGCAAT | |
| | AAACGAAAGTTTAACTAAGCTATACTAACCCCAGGGTTGGTCAATTTCG | |
| | TGCCAGCCACACCCTGGAGCTAGCAAAAAAAA | |
| 17 | TAGTTATTAATAGTAATCAATTACGGGGTCATTAGTTCATAGCCCATATA | 302 |
| Ad5-Pan-CoV | TGGAGTTCCGCGTTACATAACTTACGGTAAATGGCCCGCCTGGCTGAC | |
| HexaPro- | CGCCCAACGACCCCCGCCCATTGACGTCAATAATGACGTATGTTCCCAT | |
| NSP14- | AGTAACGCCAATAGGGACTTTCCATTGACGTCAATGGGTGGAGTATTTA | |
| NSP15 | CGGTAAACTGCCCACTTGGCAGTACATCAAGTGTATCATATGCCAAGTA | |
| | CGCCCCCTATTGACGTCAATGACGGTAAATGGCCCGCCTGGCATTATG | |
| | CCCAGTACATGACCTTATGGGACTTTCCTACTTGGCAGTACATCTACGT | |
| | ATTAGTCATCGCTATTACCATGGTGATGCGGTTTTGGCAGTACATCAAT | |
| | GGGCGTGGATAGCGGTTTGACTCACGGGGATTTCCAAGTCTCCACCCC | |
| | ATTGACGTCAATGGGAGTTTGTTTTGGCACCAAAATCAACGGGACTTTC | |
| | CAAAATGTCGTAACAACTCCGCCCCATTGACGCAAATGGGCGGTAGGC | |
| | GTGTACGGTGGGAGGTCTATATAAGCAGAGCTGGTTTAGTGAACCGTC | |
| | AGATCGAGAATAAACTAGTATTCTTCTGGTCCCCACAGACTCAGAGAGA | |
| | ACCCGCCACCATGTTCGTGTTCCTGGTGCTGCTGCCCCTGGTGAGCAG | |
| | CCAGTGCGTGAACCTGACCACCAGGACCCAGCTGCCCCCCGCCTACA | |
| | CCAACAGCTTCACCAGGGGCGTGTACTACCCCGACAAGGTGTTCAGGA | |
| | GCAGCGTGCTGCACAGCACCCAGGACCTGTTCCTGCCCTTCTTCAGCA | |
| | ACGTGACCTGGTTCCACGCCATCCACGTGAGCGGCACCAACGGCACCA | |
| | AGAGGTTCGACAACCCCGTGCTGCCCTTCAACGACGGCGTGTACTTCG | |
| | CCAGCACCGAGAAGAGCAACATCATCAGGGGCTGGATCTTCGGCACCA | |
| | CCCTGGACAGCAAGACCCAGAGCCTGCTGATCGTGAACAACGCCACCA | |
| | ACGTGGTGATCAAGGTGTGCGAGTTCCAGTTCTGCAACGACCCCTTCC | |
| | TGGGCGTGTACTACCACAAGAACAACAAGAGCTGGATGGAGAGCGAGT | |
| | TCAGGGTGTACAGCAGCGCCAACAACTGCACCTTCGAGTACGTGAGCC | |
| | AGCCCTTCCTGATGGACCTGGAGGGCAAGCAGGGCAACTTCAAGAACC | |
| | TGAGGGAGTTCGTGTTCAAGAACATCGACGGCTACTTCAAGATCTACAG | |
| | CAAGCACACCCCCATCAACCTGGTGAGGGACCTGCCCCAGGGCTTCAG | |
| | CGCCCTGGAGCCCCTGGTGGACCTGCCCATCGGCATCAACATCACCAG | |
| | GTTCCAGACCCTGCTGGCCCTGCACAGGAGCTACCTGACCCCCGGCG | |
| | ACAGCAGCAGCGGCTGGACCGCCGGCGCCGCCGCCTACTACGTGGGC | |
| | TACCTGCAGCCCAGGACCTTCCTGCTGAAGTACAACGAGAACGGCACC | |
| | ATCACCGACGCCGTGGACTGCGCCCTGGACCCCCTGAGCGAGACCAA | |
| | GTGCACCCTGAAGAGCTTCACCGTGGAGAAGGGCATCTACCAGACCAG | |
| | CAACTTCAGGGTGCAGCCCACCGAGAGCATCGTGAGGTTCCCCAACAT | |
| | CACCAACCTGTGCCCCTTCGGCGAGGTGTTCAACGCCACCAGGTTCGC | |
| | CAGCGTGTACGCCTGGAACAGGAAGAGGATCAGCAACTGCGTGGCCG | |
| | ACTACAGCGTGCTGTACAACAGCGCCAGCTTCAGCACCTTCAAGTGCT | |
| | ACGGCGTGAGCCCCACCAAGCTGAACGACCTGTGCTTCACCAACGTGT | |
| | ACGCCGACAGCTTCGTGATCAGGGGCGACGAGGTGAGGCAGATCGCC | |
| | CCCGGCCAGACCGGCAAGATCGCCGACTACAACTACAAGCTGCCCGA | |
| | CGACTTCACCGGCTGCGTGATCGCCTGGAACAGCAACAACCTGGACAG | |
| | CAAGGTGGGCGGCAACTACAACTACCTGTACAGGCTGTTCAGGAAGAG | |
| | CAACCTGAAGCCCTTCGAGAGGGACATCAGCACCGAGATCTACCAGGC | |
| | CGGCAGCACCCCCTGCAACGGCGTGGAGGGCTTCAACTGCTACTTCCC | |
| | CCTGCAGAGCTACGGCTTCCAGCCCACCAACGGCGTGGGCTACCAGC | |
| | CCTACAGGGTGGTGGTGCTGAGCTTCGAGCTGCTGCACGCCCCCGCC | |
| | ACCGTGTGCGGCCCCAAGAAGAGCACCAACCTGGTGAAGAACAAGTGC | |
| | GTGAACTTCAACTTCAACGGCCTGACCGGCACCGGCGTGCTGACCGAG | |
| | AGCAACAAGAAGTTCCTGCCCTTCCAGCAGTTCGGCAGGGACATCGCC | |
| | GACACCACCGACGCCGTGAGGGACCCCCAGACCCTGGAGATCCTGGA | |
| | CATCACCCCCTGCAGCTTCGGCGGCGTGAGCGTGATCACCCCCGGCA | |
| | CCAACACCAGCAACCAGGTGGCCGTGCTGTACCAGGACGTGAACTGCA | |
| | CCGAGGTGCCCGTGGCCATCCACGCCGACCAGCTGACCCCCACCTGG | |
| | AGGGTGTACAGCACCGGCAGCAACGTGTTCCAGACCAGGGCCGGCTG | |
| | CCTGATCGGCGCCGAGCACGTGAACAACAGCTACGAGTGCGACATCCC | |
| | CATCGGCGCCGGCATCTGCGCCAGCTACCAGACCCAGACCAACAGCC | |
| | CCGGCAGCGCCAGCAGCGTGGCCAGCCAGAGCATCATCGCCTACACC | |
| | ATGAGCCTGGGCGCCGAGAACAGCGTGGCCTACAGCAACAACAGCAT | |
| | CGCCATCCCCACCAACTTCACCATCAGCGTGACCACCGAGATCCTGCC | |
| | CGTGAGCATGACCAAGACCAGCGTGGACTGCACCATGTACATCTGCGG | |
| | CGACAGCACCGAGTGCAGCAACCTGCTGCTGCAGTACGGCAGCTTCTG | |
| | CACCCAGCTGAACAGGGCCCTGACCGGCATCGCCGTGGAGCAGGACA | |
| | AGAACACCCAGGAGGTGTTCGCCCAGGTGAAGCAGATCTACAAGACCC | |
| | CCCCCATCAAGGACTTCGGCGGCTTCAACTTCAGCCAGATCCTGCCCG | |
| | ACCCCAGCAAGCCCAGCAAGAGGAGCCCCATCGAGGACCTGCTGTTCA | |
| | ACAAGGTGACCCTGGCCGACGCCGGCTTCATCAAGCAGTACGGCGACT | |
| | GCCTGGGCGACATCGCCGCCAGGGACCTGATCTGCGCCCAGAAGTTC | |
| | AACGGCCTGACCGTGCTGCCCCCCCTGCTGACCGACGAGATGATCGC | |
| | CCAGTACACCAGCGCCCTGCTGGCCGGCACCATCACCAGCGGCTGGA | |
| | CCTTCGGCGCCGGCCCCGCCCTGCAGATCCCCTTCCCCATGCAGATG | |
| | GCCTACAGGTTCAACGGCATCGGCGTGACCCAGAACGTGCTGTACGAG | |
| | AACCAGAAGCTGATCGCCAACCAGTTCAACAGCGCCATCGGCAAGATC | |
| | CAGGACAGCCTGAGCAGCACCCCCAGCGCCCTGGGCAAGCTGCAGGA | |
| | CGTGGTGAACCAGAACGCCCAGGCCCTGAACACCCTGGTGAAGCAGC | |
| | TGAGCAGCAACTTCGGCGCCATCAGCAGCGTGCTGAACGACATCCTGA | |
| | GCAGGCTGGACCCCCCCGAGGCCGAGGTGCAGATCGACAGGCTGATC | |
| | ACCGGCAGGCTGCAGAGCCTGCAGACCTACGTGACCCAGCAGCTGAT | |
| | CAGGGCCGCCGAGATCAGGGCCAGCGCCAACCTGGCCGCCACCAAGA | |
| | TGAGCGAGTGCGTGCTGGGCCAGAGCAAGAGGGTGGACTTCTGCGGC | |
| | AAGGGCTACCACCTGATGAGCTTCCCCCAGAGCGCCCCCCACGGCGT | |
| | GGTGTTCCTGCACGTGACCTACGTGCCCGCCCAGGAGAAGAACTTCAC | |
| | CACCGCCCCCGCCATCTGCCACGACGGCAAGGCCCACTTCCCCAGGG | |
| | AGGGCGTGTTCGTGAGCAACGGCACCCACTGGTTCGTGACCCAGAGG | |
| | AACTTCTACGAGCCCCAGATCATCACCACCGACAACACCTTCGTGAGC | |
| | GGCAACTGCGACGTGGTGATCGGCATCGTGAACAACACCGTGTACGAC | |
| | CCCCTGCAGCCCGAGCTGGACAGCTTCAAGGAGGAGCTGGACAAGTA | |
| | CTTCAAGAACCACACCAGCCCCGACGTGGACCTGGGCGACATCAGCG | |
| | GCATCAACGCCAGCGTGGTGAACATCCAGAAGGAGATCGACAGGCTGA | |
| | ACGAGGTGGCCAAGAACCTGAACGAGAGCCTGATCGACCTGCAGGAG | |
| | CTGGGCAAGTACGAGCAGTACATCAAGTGGCCCTGGTACATCTGGCTG | |
| | GGCTTCATCGCCGGCCTGATCGCCATCGTGATGGTGACCATCATGCTG | |
| | TGCTGCATGACCAGCTGCTGCAGCTGCCTGAAGGGCTGCTGCAGCTGC | |
| | GGCAGCTGCTGCAAGTTCGACGAGGACGACAGCGAGCCCGTGCTGAA | |
| | GGGCGTGAAGCTGCACTACACCGGAAGCGGAGCCACGAACTTCTCTCT | |
| | GTTAAAGCAAGCAGGAGATGTTGAAGAAAACCCCGGGCCTGCCGAGAA | |
| | CGTGACCGGCCTGTTCAAGGACTGCAGCAAGGTGATCACCGGCCTGCA | |
| | CCCCACCCAGGCCCCCACCCACCTGAGCGTGGACACCAAGTTCAAGAC | |
| | CGAGGGCCTGTGCGTGGACATCCCCGGCATCCCCAAGGACATGACCT | |
| | ACAGGAGGCTGATCAGCATGATGGGCTTCAAGATGAACTACCAGGTGA | |
| | ACGGCTACCCCAACATGTTCATCACCAGGGAGGAGGCCATCAGGCACG | |
| | TGAGGGCCTGGATCGGCTTCGACGTGGAGGGCTGCCACGCCACCAGG | |
| | GAGGCCGTGGGCACCAACCTGCCCCTGCAGCTGGGCTTCAGCACCGG | |
| | CGTGAACCTGGTGGCCGTGCCCACCGGCTACGTGGACACCCCCAACA | |
| | ACACCGACTTCAGCAGGGTGAGCGCCAAGCCCCCCCCCGGCGACCAG | |
| | TTCAAGCACCTGATCCCCCTGATGTACAAGGGCCTGCCCTGGAACGTG | |
| | GTGAGGATCAAGATCGTGCAGATGCTGAGCGACACCCTGAAGAACCTG | |
| | AGCGACAGGGTGGTGTTCGTGCTGTGGGCCCACGGCTTCGAGCTGAC | |
| | CAGCATGAAGTACTTCGTGAAGATCGGCCCCGAGAGGACCTGCTGCCT | |
| | GTGCGACAGGAGGGCCACCTGCTTCAGCACCGCCAGCGACACCTACG | |
| | CCTGCTGGCACCACAGCATCGGCTTCGACTACGTGTACAACCCCTTCA | |
| | TGATCGACGTGCAGCAGTGGGGCTTCACCGGCAACCTGCAGAGCAAC | |
| | CACGACCTGTACTGCCAGGTGCACGGCAACGCCCACGTGGCCAGCTG | |
| | CGACGCCATCATGACCAGGTGCCTGGCCGTGCACGAGTGCTTCGTGAA | |
| | GAGGGTGGACTGGACCATCGAGTACCCCATCATCGGCGACGAGCTGA | |
| | AGATCAACGCCGCCTGCAGGAAGGTGCAGCACATGGTGGTGAAGGCC | |
| | GCCCTGCTGGCCGACAAGTTCCCCGTGCTGCACGACATCGGCAACCC | |
| | CAAGGCCATCAAGTGCGTGCCCCAGGCCGACGTGGAGTGGAAGTTCTA | |
| | CGACGCCCAGCCCTGCAGCGACAAGGCCTACAAGATCGAGGAGCTGT | |
| | TCTACAGCTACGCCACCCACAGCGACAAGTTCACCGACGGCGTGTGCC | |
| | TGTTCTGGAACTGCAACGTGGACAGGTACCCCGCCAACAGCATCGTGT | |
| | GCAGGTTCGACACCAGGGTGCTGAGCAACCTGAACCTGCCCGGCTGC | |
| | GACGGCGGCAGCCTGTACGTGAACAAGCACGCCTTCCACACCCCCGC | |
| | CTTCGACAAGAGCGCCTTCGTGAACCTGAAGCAGCTGCCCTTCTTCTAC | |
| | TACAGCGACAGCCCCTGCGAGAGCCACGGCAAGCAGGTGGTGAGCGA | |
| | CATCGACTACGTGCCCCTGAAGAGCGCCACCTGCATCACCAGGTGCAA | |
| | CCTGGGCGGCGCCGTGTGCAGGCACCACGCCAACGAGTACAGGCTGT | |
| | ACCTGGACGCCTACAACATGATGATCAGCGCCGGCTTCAGCCTGTGGG | |
| | TGTACAAGCAGTTCGACACCTACAACCTGTGGAACACCTTCACCAGGCT | |
| | GCAGGGAAGCGGAGAGGGCAGGGGAAGTCTTCTAACATGCGGGGACG | |
| | TGGAGGAAAATCCCGGCCCCAGCCTGGAGAACGTGGCCTTCAACGTG | |
| | GTGAACAAGGGCCACTTCGACGGCCAGCAGGGCGAGGTGCCCGTGAG | |
| | CATCATCAACAACACCGTGTACACCAAGGTGGACGGCGTGGACGTGGA | |
| | GCTGTTCGAGAACAAGACCACCCTGCCCGTGAACGTGGCCTTCGAGCT | |
| | GTGGGCCAAGAGGAACATCAAGCCCGTGCCCGAGGTGAAGATCCTGA | |
| | ACAACCTGGGCGTGGACATCGCCGCCAACACCGTGATCTGGGACTACA | |
| | AGAGGGACGCCCCCGCCCACATCAGCACCATCGGCGTGTGCAGCATG | |
| | ACCGACATCGCCAAGAAGCCCACCGAGACCATCTGCGCCCCCCTGACC | |
| | GTGTTCTTCGACGGCAGGGTGGACGGCCAGGTGGACCTGTTCAGGAA | |
| | CGCCAGGAACGGCGTGCTGATCACCGAGGGCAGCGTGAAGGGCCTGC | |
| | AGCCCAGCGTGGGCCCCAAGCAGGCCAGCCTGAACGGCGTGACCCTG | |
| | ATCGGCGAGGCCGTGAAGACCCAGTTCAACTACTACAAGAAGGTGGAC | |
| | GGCGTGGTGCAGCAGCTGCCCGAGACCTACTTCACCCAGAGCAGGAA | |
| | CCTGCAGGAGTTCAAGCCCAGGAGCCAGATGGAGATCGACTTCCTGGA | |
| | GCTGGCCATGGACGAGTTCATCGAGAGGTACAAGCTGGAGGGCTACG | |
| | CCTTCGAGCACATCGTGTACGGCGACTTCAGCCACAGCCAGCTGGGCG | |
| | GCCTGCACCTGCTGATCGGCCTGGCCAAGAGGTTCAAGGAGAGCCCC | |
| | TTCGAGCTGGAGGACTTCATCCCCATGGACAGCACCGTGAAGAACTAC | |
| | TTCATCACCGACGCCCAGACCGGCAGCAGCAAGTGCGTGTGCAGCGT | |
| | GATCGACCTGCTGCTGGACGACTTCGTGGAGATCATCAAGAGCCAGGA | |
| | CCTGAGCGTGGTGAGCAAGGTGGTGAAGGTGACCATCGACTACACCGA | |
| | GATCAGCTTCATGCTGTGGTGCAAGGACGGCCACGTGGAGACCTTCTA | |
| | CCCCAAGCTGCAGTGATGACTCGAGCTGGTACTGCATGCACGCAATGC | |
| | TAGCTGCCCCTTTCCCGTCCTGGGTACCCCGAGTCTCCCCCGACCTCG | |
| | GGTCCCAGGTATGCTCCCACCTCCACCTGCCCCACTCACCACCTCTGC | |
| | TAGTTCCAGACACCTCCCAAGCACGCAGCAATGCAGCTCAAAACGCTTA | |
| | GCCTAGCCACACCCCCACGGGAAACAGCAGTGATTAACCTTTAGCAAT | |
| | AAACGAAAGTTTAACTAAGCTATACTAACCCCAGGGTTGGTCAATTTCG | |
| | TGCCAGCCACACCCTGGAGCTAGCAAAAAAAA | |
| 18 | TAGTTATTAATAGTAATCAATTACGGGGTCATTAGTTCATAGCCCATATA | 303 |
| Ad5-Pan-CoV | TGGAGTTCCGCGTTACATAACTTACGGTAAATGGCCCGCCTGGCTGAC | |
| Spike-NSP16 | CGCCCAACGACCCCCGCCCATTGACGTCAATAATGACGTATGTTCCCAT | |
| | AGTAACGCCAATAGGGACTTTCCATTGACGTCAATGGGTGGAGTATTTA | |
| | CGGTAAACTGCCCACTTGGCAGTACATCAAGTGTATCATATGCCAAGTA | |
| | CGCCCCCTATTGACGTCAATGACGGTAAATGGCCCGCCTGGCATTATG | |
| | CCCAGTACATGACCTTATGGGACTTTCCTACTTGGCAGTACATCTACGT | |
| | ATTAGTCATCGCTATTACCATGGTGATGCGGTTTTGGCAGTACATCAAT | |
| | GGGCGTGGATAGCGGTTTGACTCACGGGGATTTCCAAGTCTCCACCCC | |
| | ATTGACGTCAATGGGAGTTTGTTTTGGCACCAAAATCAACGGGACTTTC | |
| | CAAAATGTCGTAACAACTCCGCCCCATTGACGCAAATGGGCGGTAGGC | |
| | GTGTACGGTGGGAGGTCTATATAAGCAGAGCTGGTTTAGTGAACCGTC | |
| | AGATCGAGAATAAACTAGTATTCTTCTGGTCCCCACAGACTCAGAGAGA | |
| | ACCCGCCACCATGTTCGTGTTCCTGGTGCTGCTGCCCCTGGTGAGCAG | |
| | CCAGTGCGTGAACCTGACCACCAGGACCCAGCTGCCCCCCGCCTACA | |
| | CCAACAGCTTCACCAGGGGCGTGTACTACCCCGACAAGGTGTTCAGGA | |
| | GCAGCGTGCTGCACAGCACCCAGGACCTGTTCCTGCCCTTCTTCAGCA | |
| | ACGTGACCTGGTTCCACGCCATCCACGTGAGCGGCACCAACGGCACCA | |
| | AGAGGTTCGACAACCCCGTGCTGCCCTTCAACGACGGCGTGTACTTCG | |
| | CCAGCACCGAGAAGAGCAACATCATCAGGGGCTGGATCTTCGGCACCA | |
| | CCCTGGACAGCAAGACCCAGAGCCTGCTGATCGTGAACAACGCCACCA | |
| | ACGTGGTGATCAAGGTGTGCGAGTTCCAGTTCTGCAACGACCCCTTCC | |
| | TGGGCGTGTACTACCACAAGAACAACAAGAGCTGGATGGAGAGCGAGT | |
| | TCAGGGTGTACAGCAGCGCCAACAACTGCACCTTCGAGTACGTGAGCC | |
| | AGCCCTTCCTGATGGACCTGGAGGGCAAGCAGGGCAACTTCAAGAACC | |
| | TGAGGGAGTTCGTGTTCAAGAACATCGACGGCTACTTCAAGATCTACAG | |
| | CAAGCACACCCCCATCAACCTGGTGAGGGACCTGCCCCAGGGCTTCAG | |
| | CGCCCTGGAGCCCCTGGTGGACCTGCCCATCGGCATCAACATCACCAG | |
| | GTTCCAGACCCTGCTGGCCCTGCACAGGAGCTACCTGACCCCCGGCG | |
| | ACAGCAGCAGCGGCTGGACCGCCGGCGCCGCCGCCTACTACGTGGGC | |
| | TACCTGCAGCCCAGGACCTTCCTGCTGAAGTACAACGAGAACGGCACC | |
| | ATCACCGACGCCGTGGACTGCGCCCTGGACCCCCTGAGCGAGACCAA | |
| | GTGCACCCTGAAGAGCTTCACCGTGGAGAAGGGCATCTACCAGACCAG | |
| | CAACTTCAGGGTGCAGCCCACCGAGAGCATCGTGAGGTTCCCCAACAT | |
| | CACCAACCTGTGCCCCTTCGGCGAGGTGTTCAACGCCACCAGGTTCGC | |
| | CAGCGTGTACGCCTGGAACAGGAAGAGGATCAGCAACTGCGTGGCCG | |
| | ACTACAGCGTGCTGTACAACAGCGCCAGCTTCAGCACCTTCAAGTGCT | |
| | ACGGCGTGAGCCCCACCAAGCTGAACGACCTGTGCTTCACCAACGTGT | |
| | ACGCCGACAGCTTCGTGATCAGGGGCGACGAGGTGAGGCAGATCGCC | |
| | CCCGGCCAGACCGGCAAGATCGCCGACTACAACTACAAGCTGCCCGA | |
| | CGACTTCACCGGCTGCGTGATCGCCTGGAACAGCAACAACCTGGACAG | |
| | CAAGGTGGGCGGCAACTACAACTACCTGTACAGGCTGTTCAGGAAGAG | |
| | CAACCTGAAGCCCTTCGAGAGGGACATCAGCACCGAGATCTACCAGGC | |
| | CGGCAGCACCCCCTGCAACGGCGTGGAGGGCTTCAACTGCTACTTCCC | |
| | CCTGCAGAGCTACGGCTTCCAGCCCACCAACGGCGTGGGCTACCAGC | |
| | CCTACAGGGTGGTGGTGCTGAGCTTCGAGCTGCTGCACGCCCCCGCC | |
| | ACCGTGTGCGGCCCCAAGAAGAGCACCAACCTGGTGAAGAACAAGTGC | |
| | GTGAACTTCAACTTCAACGGCCTGACCGGCACCGGCGTGCTGACCGAG | |
| | AGCAACAAGAAGTTCCTGCCCTTCCAGCAGTTCGGCAGGGACATCGCC | |
| | GACACCACCGACGCCGTGAGGGACCCCCAGACCCTGGAGATCCTGGA | |
| | CATCACCCCCTGCAGCTTCGGCGGCGTGAGCGTGATCACCCCCGGCA | |
| | CCAACACCAGCAACCAGGTGGCCGTGCTGTACCAGGACGTGAACTGCA | |
| | CCGAGGTGCCCGTGGCCATCCACGCCGACCAGCTGACCCCCACCTGG | |
| | AGGGTGTACAGCACCGGCAGCAACGTGTTCCAGACCAGGGCCGGCTG | |
| | CCTGATCGGCGCCGAGCACGTGAACAACAGCTACGAGTGCGACATCCC | |
| | CATCGGCGCCGGCATCTGCGCCAGCTACCAGACCCAGACCAACAGCC | |
| | CCAGGAGGGCCAGGAGCGTGGCCAGCCAGAGCATCATCGCCTACACC | |
| | ATGAGCCTGGGCGCCGAGAACAGCGTGGCCTACAGCAACAACAGCAT | |
| | CGCCATCCCCACCAACTTCACCATCAGCGTGACCACCGAGATCCTGCC | |
| | CGTGAGCATGACCAAGACCAGCGTGGACTGCACCATGTACATCTGCGG | |
| | CGACAGCACCGAGTGCAGCAACCTGCTGCTGCAGTACGGCAGCTTCTG | |
| | CACCCAGCTGAACAGGGCCCTGACCGGCATCGCCGTGGAGCAGGACA | |
| | AGAACACCCAGGAGGTGTTCGCCCAGGTGAAGCAGATCTACAAGACCC | |
| | CCCCCATCAAGGACTTCGGCGGCTTCAACTTCAGCCAGATCCTGCCCG | |
| | ACCCCAGCAAGCCCAGCAAGAGGAGCTTCATCGAGGACCTGCTGTTCA | |
| | ACAAGGTGACCCTGGCCGACGCCGGCTTCATCAAGCAGTACGGCGACT | |
| | GCCTGGGCGACATCGCCGCCAGGGACCTGATCTGCGCCCAGAAGTTC | |
| | AACGGCCTGACCGTGCTGCCCCCCCTGCTGACCGACGAGATGATCGC | |
| | CCAGTACACCAGCGCCCTGCTGGCCGGCACCATCACCAGCGGCTGGA | |
| | CCTTCGGCGCCGGCGCCGCCCTGCAGATCCCCTTCGCCATGCAGATG | |
| | GCCTACAGGTTCAACGGCATCGGCGTGACCCAGAACGTGCTGTACGAG | |
| | AACCAGAAGCTGATCGCCAACCAGTTCAACAGCGCCATCGGCAAGATC | |
| | CAGGACAGCCTGAGCAGCACCGCCAGCGCCCTGGGCAAGCTGCAGGA | |
| | CGTGGTGAACCAGAACGCCCAGGCCCTGAACACCCTGGTGAAGCAGC | |
| | TGAGCAGCAACTTCGGCGCCATCAGCAGCGTGCTGAACGACATCCTGA | |
| | GCAGGCTGGACCCCCCCGAGGCCGAGGTGCAGATCGACAGGCTGATC | |
| | ACCGGCAGGCTGCAGAGCCTGCAGACCTACGTGACCCAGCAGCTGAT | |
| | CAGGGCCGCCGAGATCAGGGCCAGCGCCAACCTGGCCGCCACCAAGA | |
| | TGAGCGAGTGCGTGCTGGGCCAGAGCAAGAGGGTGGACTTCTGCGGC | |
| | AAGGGCTACCACCTGATGAGCTTCCCCCAGAGCGCCCCCCACGGCGT | |
| | GGTGTTCCTGCACGTGACCTACGTGCCCGCCCAGGAGAAGAACTTCAC | |
| | CACCGCCCCCGCCATCTGCCACGACGGCAAGGCCCACTTCCCCAGGG | |
| | AGGGCGTGTTCGTGAGCAACGGCACCCACTGGTTCGTGACCCAGAGG | |
| | AACTTCTACGAGCCCCAGATCATCACCACCGACAACACCTTCGTGAGC | |
| | GGCAACTGCGACGTGGTGATCGGCATCGTGAACAACACCGTGTACGAC | |
| | CCCCTGCAGCCCGAGCTGGACAGCTTCAAGGAGGAGCTGGACAAGTA | |
| | CTTCAAGAACCACACCAGCCCCGACGTGGACCTGGGCGACATCAGCG | |
| | GCATCAACGCCAGCGTGGTGAACATCCAGAAGGAGATCGACAGGCTGA | |
| | ACGAGGTGGCCAAGAACCTGAACGAGAGCCTGATCGACCTGCAGGAG | |
| | CTGGGCAAGTACGAGCAGTACATCAAGTGGCCCTGGTACATCTGGCTG | |
| | GGCTTCATCGCCGGCCTGATCGCCATCGTGATGGTGACCATCATGCTG | |
| | TGCTGCATGACCAGCTGCTGCAGCTGCCTGAAGGGCTGCTGCAGCTGC | |
| | GGCAGCTGCTGCAAGTTCGACGAGGACGACAGCGAGCCCGTGCTGAA | |
| | GGGCGTGAAGCTGCACTACACCGGAAGCGGAGCCACGAACTTCTCTCT | |
| | GTTAAAGCAAGCAGGAGATGTTGAAGAAAACCCCGGGCCTAGCAGCCA | |
| | GGCCTGGCAGCCCGGCGTGGCCATGCCCAACCTGTACAAGATGCAGA | |
| | GGATGCTGCTGGAGAAGTGCGACCTGCAGAACTACGGCGACAGCGCC | |
| | ACCCTGCCCAAGGGCATCATGATGAACGTGGCCAAGTACACCCAGCTG | |
| | TGCCAGTACCTGAACACCCTGACCCTGGCCGTGCCCTACAACATGAGG | |
| | GTGATCCACTTCGGCGCCGGCAGCGACAAGGGCGTGGCCCCCGGCAC | |
| | CGCCGTGCTGAGGCAGTGGCTGCCCACCGGCACCCTGCTGGTGGACA | |
| | GCGACCTGAACGACTTCGTGAGCGACGCCGACAGCACCCTGATCGGC | |
| | GACTGCGCCACCGTGCACACCGCCAACAAGTGGGACCTGATCATCAGC | |
| | GACATGTACGACCCCAAGACCAAGAACGTGACCAAGGAGAACGACAGC | |
| | AAGGAGGGCTTCTTCACCTACATCTGCGGCTTCATCCAGCAGAAGCTG | |
| | GCCCTGGGCGGCAGCGTGGCCATCAAGATCACCGAGCACAGCTGGAA | |
| | CGCCGACCTGTACAAGCTGATGGGCCACTTCGCCTGGTGGACCGCCTT | |
| | CGTGACCAACGTGAACGCCAGCAGCAGCGAGGCCTTCCTGATCGGCT | |
| | GCAACTACCTGGGCAAGCCCAGGGAGCAGATCGACGGCTACGTGATG | |
| | CACGCCAACTACATCTTCTGGAGGAACACCAACCCCATCCAGCTGAGC | |
| | AGCTACAGCCTGTTCGACATGAGCAAGTTCCCCCTGAAGCTGAGGGGC | |
| | ACCGCCGTGATGAGCCTGAAGGAGGGCCAGATCAACGACATGATCCTG | |
| | AGCCTGCTGAGCAAGGGCAGGCTGATCATCAGGGAGAACAACAGGGT | |
| | GGTGATCAGCAGCGACGTGCTGGTGAACAACTGATGACTCGAGCTGGT | |
| | ACTGCATGCACGCAATGCTAGCTGCCCCTTTCCCGTCCTGGGTACCCC | |
| | GAGTCTCCCCCGACCTCGGGTCCCAGGTATGCTCCCACCTCCACCTGC | |
| | CCCACTCACCACCTCTGCTAGTTCCAGACACCTCCCAAGCACGCAGCA | |
| | ATGCAGCTCAAAACGCTTAGCCTAGCCACACCCCCACGGGAAACAGCA | |
| | GTGATTAACCTTTAGCAATAAACGAAAGTTTAACTAAGCTATACTAACCC | |
| | CAGGGTTGGTCAATTTCGTGCCAGCCACACCCTGGAGCTAGCAAAAAA | |
| | AA | |
| 19 | TAGTTATTAATAGTAATCAATTACGGGGTCATTAGTTCATAGCCCATATA | 304 |
| Ad5-Pan-CoV | TGGAGTTCCGCGTTACATAACTTACGGTAAATGGCCCGCCTGGCTGAC | |
| HexaPro- | CGCCCAACGACCCCCGCCCATTGACGTCAATAATGACGTATGTTCCCAT | |
| NSP16 | AGTAACGCCAATAGGGACTTTCCATTGACGTCAATGGGTGGAGTATTTA | |
| | CGGTAAACTGCCCACTTGGCAGTACATCAAGTGTATCATATGCCAAGTA | |
| | CGCCCCCTATTGACGTCAATGACGGTAAATGGCCCGCCTGGCATTATG | |
| | CCCAGTACATGACCTTATGGGACTTTCCTACTTGGCAGTACATCTACGT | |
| | ATTAGTCATCGCTATTACCATGGTGATGCGGTTTTGGCAGTACATCAAT | |
| | GGGCGTGGATAGCGGTTTGACTCACGGGGATTTCCAAGTCTCCACCCC | |
| | ATTGACGTCAATGGGAGTTTGTTTTGGCACCAAAATCAACGGGACTTTC | |
| | CAAAATGTCGTAACAACTCCGCCCCATTGACGCAAATGGGCGGTAGGC | |
| | GTGTACGGTGGGAGGTCTATATAAGCAGAGCTGGTTTAGTGAACCGTC | |
| | AGATCGAGAATAAACTAGTATTCTTCTGGTCCCCACAGACTCAGAGAGA | |
| | ACCCGCCACCATGTTCGTGTTCCTGGTGCTGCTGCCCCTGGTGAGCAG | |
| | CCAGTGCGTGAACCTGACCACCAGGACCCAGCTGCCCCCCGCCTACA | |
| | CCAACAGCTTCACCAGGGGCGTGTACTACCCCGACAAGGTGTTCAGGA | |
| | GCAGCGTGCTGCACAGCACCCAGGACCTGTTCCTGCCCTTCTTCAGCA | |
| | ACGTGACCTGGTTCCACGCCATCCACGTGAGCGGCACCAACGGCACCA | |
| | AGAGGTTCGACAACCCCGTGCTGCCCTTCAACGACGGCGTGTACTTCG | |
| | CCAGCACCGAGAAGAGCAACATCATCAGGGGCTGGATCTTCGGCACCA | |
| | CCCTGGACAGCAAGACCCAGAGCCTGCTGATCGTGAACAACGCCACCA | |
| | ACGTGGTGATCAAGGTGTGCGAGTTCCAGTTCTGCAACGACCCCTTCC | |
| | TGGGCGTGTACTACCACAAGAACAACAAGAGCTGGATGGAGAGCGAGT | |
| | TCAGGGTGTACAGCAGCGCCAACAACTGCACCTTCGAGTACGTGAGCC | |
| | AGCCCTTCCTGATGGACCTGGAGGGCAAGCAGGGCAACTTCAAGAACC | |
| | TGAGGGAGTTCGTGTTCAAGAACATCGACGGCTACTTCAAGATCTACAG | |
| | CAAGCACACCCCCATCAACCTGGTGAGGGACCTGCCCCAGGGCTTCAG | |
| | CGCCCTGGAGCCCCTGGTGGACCTGCCCATCGGCATCAACATCACCAG | |
| | GTTCCAGACCCTGCTGGCCCTGCACAGGAGCTACCTGACCCCCGGCG | |
| | ACAGCAGCAGCGGCTGGACCGCCGGCGCCGCCGCCTACTACGTGGGC | |
| | TACCTGCAGCCCAGGACCTTCCTGCTGAAGTACAACGAGAACGGCACC | |
| | ATCACCGACGCCGTGGACTGCGCCCTGGACCCCCTGAGCGAGACCAA | |
| | GTGCACCCTGAAGAGCTTCACCGTGGAGAAGGGCATCTACCAGACCAG | |
| | CAACTTCAGGGTGCAGCCCACCGAGAGCATCGTGAGGTTCCCCAACAT | |
| | CACCAACCTGTGCCCCTTCGGCGAGGTGTTCAACGCCACCAGGTTCGC | |
| | CAGCGTGTACGCCTGGAACAGGAAGAGGATCAGCAACTGCGTGGCCG | |
| | ACTACAGCGTGCTGTACAACAGCGCCAGCTTCAGCACCTTCAAGTGCT | |
| | ACGGCGTGAGCCCCACCAAGCTGAACGACCTGTGCTTCACCAACGTGT | |
| | ACGCCGACAGCTTCGTGATCAGGGGCGACGAGGTGAGGCAGATCGCC | |
| | CCCGGCCAGACCGGCAAGATCGCCGACTACAACTACAAGCTGCCCGA | |
| | CGACTTCACCGGCTGCGTGATCGCCTGGAACAGCAACAACCTGGACAG | |
| | CAAGGTGGGCGGCAACTACAACTACCTGTACAGGCTGTTCAGGAAGAG | |
| | CAACCTGAAGCCCTTCGAGAGGGACATCAGCACCGAGATCTACCAGGC | |
| | CGGCAGCACCCCCTGCAACGGCGTGGAGGGCTTCAACTGCTACTTCCC | |
| | CCTGCAGAGCTACGGCTTCCAGCCCACCAACGGCGTGGGCTACCAGC | |
| | CCTACAGGGTGGTGGTGCTGAGCTTCGAGCTGCTGCACGCCCCCGCC | |
| | ACCGTGTGCGGCCCCAAGAAGAGCACCAACCTGGTGAAGAACAAGTGC | |
| | GTGAACTTCAACTTCAACGGCCTGACCGGCACCGGCGTGCTGACCGAG | |
| | AGCAACAAGAAGTTCCTGCCCTTCCAGCAGTTCGGCAGGGACATCGCC | |
| | GACACCACCGACGCCGTGAGGGACCCCCAGACCCTGGAGATCCTGGA | |
| | CATCACCCCCTGCAGCTTCGGCGGCGTGAGCGTGATCACCCCCGGCA | |
| | CCAACACCAGCAACCAGGTGGCCGTGCTGTACCAGGACGTGAACTGCA | |
| | CCGAGGTGCCCGTGGCCATCCACGCCGACCAGCTGACCCCCACCTGG | |
| | AGGGTGTACAGCACCGGCAGCAACGTGTTCCAGACCAGGGCCGGCTG | |
| | CCTGATCGGCGCCGAGCACGTGAACAACAGCTACGAGTGCGACATCCC | |
| | CATCGGCGCCGGCATCTGCGCCAGCTACCAGACCCAGACCAACAGCC | |
| | CCGGCAGCGCCAGCAGCGTGGCCAGCCAGAGCATCATCGCCTACACC | |
| | ATGAGCCTGGGCGCCGAGAACAGCGTGGCCTACAGCAACAACAGCAT | |
| | CGCCATCCCCACCAACTTCACCATCAGCGTGACCACCGAGATCCTGCC | |
| | CGTGAGCATGACCAAGACCAGCGTGGACTGCACCATGTACATCTGCGG | |
| | CGACAGCACCGAGTGCAGCAACCTGCTGCTGCAGTACGGCAGCTTCTG | |
| | CACCCAGCTGAACAGGGCCCTGACCGGCATCGCCGTGGAGCAGGACA | |
| | AGAACACCCAGGAGGTGTTCGCCCAGGTGAAGCAGATCTACAAGACCC | |
| | CCCCCATCAAGGACTTCGGCGGCTTCAACTTCAGCCAGATCCTGCCCG | |
| | ACCCCAGCAAGCCCAGCAAGAGGAGCCCCATCGAGGACCTGCTGTTCA | |
| | ACAAGGTGACCCTGGCCGACGCCGGCTTCATCAAGCAGTACGGCGACT | |
| | GCCTGGGCGACATCGCCGCCAGGGACCTGATCTGCGCCCAGAAGTTC | |
| | AACGGCCTGACCGTGCTGCCCCCCCTGCTGACCGACGAGATGATCGC | |
| | CCAGTACACCAGCGCCCTGCTGGCCGGCACCATCACCAGCGGCTGGA | |
| | CCTTCGGCGCCGGCCCCGCCCTGCAGATCCCCTTCCCCATGCAGATG | |
| | GCCTACAGGTTCAACGGCATCGGCGTGACCCAGAACGTGCTGTACGAG | |
| | AACCAGAAGCTGATCGCCAACCAGTTCAACAGCGCCATCGGCAAGATC | |
| | CAGGACAGCCTGAGCAGCACCCCCAGCGCCCTGGGCAAGCTGCAGGA | |
| | CGTGGTGAACCAGAACGCCCAGGCCCTGAACACCCTGGTGAAGCAGC | |
| | TGAGCAGCAACTTCGGCGCCATCAGCAGCGTGCTGAACGACATCCTGA | |
| | GCAGGCTGGACCCCCCCGAGGCCGAGGTGCAGATCGACAGGCTGATC | |
| | ACCGGCAGGCTGCAGAGCCTGCAGACCTACGTGACCCAGCAGCTGAT | |
| | CAGGGCCGCCGAGATCAGGGCCAGCGCCAACCTGGCCGCCACCAAGA | |
| | TGAGCGAGTGCGTGCTGGGCCAGAGCAAGAGGGTGGACTTCTGCGGC | |
| | AAGGGCTACCACCTGATGAGCTTCCCCCAGAGCGCCCCCCACGGCGT | |
| | GGTGTTCCTGCACGTGACCTACGTGCCCGCCCAGGAGAAGAACTTCAC | |
| | CACCGCCCCCGCCATCTGCCACGACGGCAAGGCCCACTTCCCCAGGG | |
| | AGGGCGTGTTCGTGAGCAACGGCACCCACTGGTTCGTGACCCAGAGG | |
| | AACTTCTACGAGCCCCAGATCATCACCACCGACAACACCTTCGTGAGC | |
| | GGCAACTGCGACGTGGTGATCGGCATCGTGAACAACACCGTGTACGAC | |
| | CCCCTGCAGCCCGAGCTGGACAGCTTCAAGGAGGAGCTGGACAAGTA | |
| | CTTCAAGAACCACACCAGCCCCGACGTGGACCTGGGCGACATCAGCG | |
| | GCATCAACGCCAGCGTGGTGAACATCCAGAAGGAGATCGACAGGCTGA | |
| | ACGAGGTGGCCAAGAACCTGAACGAGAGCCTGATCGACCTGCAGGAG | |
| | CTGGGCAAGTACGAGCAGTACATCAAGTGGCCCTGGTACATCTGGCTG | |
| | GGCTTCATCGCCGGCCTGATCGCCATCGTGATGGTGACCATCATGCTG | |
| | TGCTGCATGACCAGCTGCTGCAGCTGCCTGAAGGGCTGCTGCAGCTGC | |
| | GGCAGCTGCTGCAAGTTCGACGAGGACGACAGCGAGCCCGTGCTGAA | |
| | GGGCGTGAAGCTGCACTACACCGGAAGCGGAGCCACGAACTTCTCTCT | |
| | GTTAAAGCAAGCAGGAGATGTTGAAGAAAACCCCGGGCCTAGCAGCCA | |
| | GGCCTGGCAGCCCGGCGTGGCCATGCCCAACCTGTACAAGATGCAGA | |
| | GGATGCTGCTGGAGAAGTGCGACCTGCAGAACTACGGCGACAGCGCC | |
| | ACCCTGCCCAAGGGCATCATGATGAACGTGGCCAAGTACACCCAGCTG | |
| | TGCCAGTACCTGAACACCCTGACCCTGGCCGTGCCCTACAACATGAGG | |
| | GTGATCCACTTCGGCGCCGGCAGCGACAAGGGCGTGGCCCCCGGCAC | |
| | CGCCGTGCTGAGGCAGTGGCTGCCCACCGGCACCCTGCTGGTGGACA | |
| | GCGACCTGAACGACTTCGTGAGCGACGCCGACAGCACCCTGATCGGC | |
| | GACTGCGCCACCGTGCACACCGCCAACAAGTGGGACCTGATCATCAGC | |
| | GACATGTACGACCCCAAGACCAAGAACGTGACCAAGGAGAACGACAGC | |
| | AAGGAGGGCTTCTTCACCTACATCTGCGGCTTCATCCAGCAGAAGCTG | |
| | GCCCTGGGCGGCAGCGTGGCCATCAAGATCACCGAGCACAGCTGGAA | |
| | CGCCGACCTGTACAAGCTGATGGGCCACTTCGCCTGGTGGACCGCCTT | |
| | CGTGACCAACGTGAACGCCAGCAGCAGCGAGGCCTTCCTGATCGGCT | |
| | GCAACTACCTGGGCAAGCCCAGGGAGCAGATCGACGGCTACGTGATG | |
| | CACGCCAACTACATCTTCTGGAGGAACACCAACCCCATCCAGCTGAGC | |
| | AGCTACAGCCTGTTCGACATGAGCAAGTTCCCCCTGAAGCTGAGGGGC | |
| | ACCGCCGTGATGAGCCTGAAGGAGGGCCAGATCAACGACATGATCCTG | |
| | AGCCTGCTGAGCAAGGGCAGGCTGATCATCAGGGAGAACAACAGGGT | |
| | GGTGATCAGCAGCGACGTGCTGGTGAACAACTGATGACTCGAGCTGGT | |
| | ACTGCATGCACGCAATGCTAGCTGCCCCTTTCCCGTCCTGGGTACCCC | |
| | GAGTCTCCCCCGACCTCGGGTCCCAGGTATGCTCCCACCTCCACCTGC | |
| | CCCACTCACCACCTCTGCTAGTTCCAGACACCTCCCAAGCACGCAGCA | |
| | ATGCAGCTCAAAACGCTTAGCCTAGCCACACCCCCACGGGAAACAGCA | |
| | GTGATTAACCTTTAGCAATAAACGAAAGTTTAACTAAGCTATACTAACCC | |
| | CAGGGTTGGTCAATTTCGTGCCAGCCACACCCTGGAGCTAGCAAAAAA | |
| | AA | |
| 20 | TAGTTATTAATAGTAATCAATTACGGGGTCATTAGTTCATAGCCCATATA | 305 |
| Ad5-Pan-CoV | TGGAGTTCCGCGTTACATAACTTACGGTAAATGGCCCGCCTGGCTGAC | |
| Spike-ORF3a | CGCCCAACGACCCCCGCCCATTGACGTCAATAATGACGTATGTTCCCAT | |
| | AGTAACGCCAATAGGGACTTTCCATTGACGTCAATGGGTGGAGTATTTA | |
| | CGGTAAACTGCCCACTTGGCAGTACATCAAGTGTATCATATGCCAAGTA | |
| | CGCCCCCTATTGACGTCAATGACGGTAAATGGCCCGCCTGGCATTATG | |
| | CCCAGTACATGACCTTATGGGACTTTCCTACTTGGCAGTACATCTACGT | |
| | ATTAGTCATCGCTATTACCATGGTGATGCGGTTTTGGCAGTACATCAAT | |
| | GGGCGTGGATAGCGGTTTGACTCACGGGGATTTCCAAGTCTCCACCCC | |
| | ATTGACGTCAATGGGAGTTTGTTTTGGCACCAAAATCAACGGGACTTTC | |
| | CAAAATGTCGTAACAACTCCGCCCCATTGACGCAAATGGGCGGTAGGC | |
| | GTGTACGGTGGGAGGTCTATATAAGCAGAGCTGGTTTAGTGAACCGTC | |
| | AGATCGAGAATAAACTAGTATTCTTCTGGTCCCCACAGACTCAGAGAGA | |
| | ACCCGCCACCATGTTCGTGTTCCTGGTGCTGCTGCCCCTGGTGAGCAG | |
| | CCAGTGCGTGAACCTGACCACCAGGACCCAGCTGCCCCCCGCCTACA | |
| | CCAACAGCTTCACCAGGGGCGTGTACTACCCCGACAAGGTGTTCAGGA | |
| | GCAGCGTGCTGCACAGCACCCAGGACCTGTTCCTGCCCTTCTTCAGCA | |
| | ACGTGACCTGGTTCCACGCCATCCACGTGAGCGGCACCAACGGCACCA | |
| | AGAGGTTCGACAACCCCGTGCTGCCCTTCAACGACGGCGTGTACTTCG | |
| | CCAGCACCGAGAAGAGCAACATCATCAGGGGCTGGATCTTCGGCACCA | |
| | CCCTGGACAGCAAGACCCAGAGCCTGCTGATCGTGAACAACGCCACCA | |
| | ACGTGGTGATCAAGGTGTGCGAGTTCCAGTTCTGCAACGACCCCTTCC | |
| | TGGGCGTGTACTACCACAAGAACAACAAGAGCTGGATGGAGAGCGAGT | |
| | TCAGGGTGTACAGCAGCGCCAACAACTGCACCTTCGAGTACGTGAGCC | |
| | AGCCCTTCCTGATGGACCTGGAGGGCAAGCAGGGCAACTTCAAGAACC | |
| | TGAGGGAGTTCGTGTTCAAGAACATCGACGGCTACTTCAAGATCTACAG | |
| | CAAGCACACCCCCATCAACCTGGTGAGGGACCTGCCCCAGGGCTTCAG | |
| | CGCCCTGGAGCCCCTGGTGGACCTGCCCATCGGCATCAACATCACCAG | |
| | GTTCCAGACCCTGCTGGCCCTGCACAGGAGCTACCTGACCCCCGGCG | |
| | ACAGCAGCAGCGGCTGGACCGCCGGCGCCGCCGCCTACTACGTGGGC | |
| | TACCTGCAGCCCAGGACCTTCCTGCTGAAGTACAACGAGAACGGCACC | |
| | ATCACCGACGCCGTGGACTGCGCCCTGGACCCCCTGAGCGAGACCAA | |
| | GTGCACCCTGAAGAGCTTCACCGTGGAGAAGGGCATCTACCAGACCAG | |
| | CAACTTCAGGGTGCAGCCCACCGAGAGCATCGTGAGGTTCCCCAACAT | |
| | CACCAACCTGTGCCCCTTCGGCGAGGTGTTCAACGCCACCAGGTTCGC | |
| | CAGCGTGTACGCCTGGAACAGGAAGAGGATCAGCAACTGCGTGGCCG | |
| | ACTACAGCGTGCTGTACAACAGCGCCAGCTTCAGCACCTTCAAGTGCT | |
| | ACGGCGTGAGCCCCACCAAGCTGAACGACCTGTGCTTCACCAACGTGT | |
| | ACGCCGACAGCTTCGTGATCAGGGGCGACGAGGTGAGGCAGATCGCC | |
| | CCCGGCCAGACCGGCAAGATCGCCGACTACAACTACAAGCTGCCCGA | |
| | CGACTTCACCGGCTGCGTGATCGCCTGGAACAGCAACAACCTGGACAG | |
| | CAAGGTGGGCGGCAACTACAACTACCTGTACAGGCTGTTCAGGAAGAG | |
| | CAACCTGAAGCCCTTCGAGAGGGACATCAGCACCGAGATCTACCAGGC | |
| | CGGCAGCACCCCCTGCAACGGCGTGGAGGGCTTCAACTGCTACTTCCC | |
| | CCTGCAGAGCTACGGCTTCCAGCCCACCAACGGCGTGGGCTACCAGC | |
| | CCTACAGGGTGGTGGTGCTGAGCTTCGAGCTGCTGCACGCCCCCGCC | |
| | ACCGTGTGCGGCCCCAAGAAGAGCACCAACCTGGTGAAGAACAAGTGC | |
| | GTGAACTTCAACTTCAACGGCCTGACCGGCACCGGCGTGCTGACCGAG | |
| | AGCAACAAGAAGTTCCTGCCCTTCCAGCAGTTCGGCAGGGACATCGCC | |
| | GACACCACCGACGCCGTGAGGGACCCCCAGACCCTGGAGATCCTGGA | |
| | CATCACCCCCTGCAGCTTCGGCGGCGTGAGCGTGATCACCCCCGGCA | |
| | CCAACACCAGCAACCAGGTGGCCGTGCTGTACCAGGACGTGAACTGCA | |
| | CCGAGGTGCCCGTGGCCATCCACGCCGACCAGCTGACCCCCACCTGG | |
| | AGGGTGTACAGCACCGGCAGCAACGTGTTCCAGACCAGGGCCGGCTG | |
| | CCTGATCGGCGCCGAGCACGTGAACAACAGCTACGAGTGCGACATCCC | |
| | CATCGGCGCCGGCATCTGCGCCAGCTACCAGACCCAGACCAACAGCC | |
| | CCAGGAGGGCCAGGAGCGTGGCCAGCCAGAGCATCATCGCCTACACC | |
| | ATGAGCCTGGGCGCCGAGAACAGCGTGGCCTACAGCAACAACAGCAT | |
| | CGCCATCCCCACCAACTTCACCATCAGCGTGACCACCGAGATCCTGCC | |
| | CGTGAGCATGACCAAGACCAGCGTGGACTGCACCATGTACATCTGCGG | |
| | CGACAGCACCGAGTGCAGCAACCTGCTGCTGCAGTACGGCAGCTTCTG | |
| | CACCCAGCTGAACAGGGCCCTGACCGGCATCGCCGTGGAGCAGGACA | |
| | AGAACACCCAGGAGGTGTTCGCCCAGGTGAAGCAGATCTACAAGACCC | |
| | CCCCCATCAAGGACTTCGGCGGCTTCAACTTCAGCCAGATCCTGCCCG | |
| | ACCCCAGCAAGCCCAGCAAGAGGAGCTTCATCGAGGACCTGCTGTTCA | |
| | ACAAGGTGACCCTGGCCGACGCCGGCTTCATCAAGCAGTACGGCGACT | |
| | GCCTGGGCGACATCGCCGCCAGGGACCTGATCTGCGCCCAGAAGTTC | |
| | AACGGCCTGACCGTGCTGCCCCCCCTGCTGACCGACGAGATGATCGC | |
| | CCAGTACACCAGCGCCCTGCTGGCCGGCACCATCACCAGCGGCTGGA | |
| | CCTTCGGCGCCGGCGCCGCCCTGCAGATCCCCTTCGCCATGCAGATG | |
| | GCCTACAGGTTCAACGGCATCGGCGTGACCCAGAACGTGCTGTACGAG | |
| | AACCAGAAGCTGATCGCCAACCAGTTCAACAGCGCCATCGGCAAGATC | |
| | CAGGACAGCCTGAGCAGCACCGCCAGCGCCCTGGGCAAGCTGCAGGA | |
| | CGTGGTGAACCAGAACGCCCAGGCCCTGAACACCCTGGTGAAGCAGC | |
| | TGAGCAGCAACTTCGGCGCCATCAGCAGCGTGCTGAACGACATCCTGA | |
| | GCAGGCTGGACCCCCCCGAGGCCGAGGTGCAGATCGACAGGCTGATC | |
| | ACCGGCAGGCTGCAGAGCCTGCAGACCTACGTGACCCAGCAGCTGAT | |
| | CAGGGCCGCCGAGATCAGGGCCAGCGCCAACCTGGCCGCCACCAAGA | |
| | TGAGCGAGTGCGTGCTGGGCCAGAGCAAGAGGGTGGACTTCTGCGGC | |
| | AAGGGCTACCACCTGATGAGCTTCCCCCAGAGCGCCCCCCACGGCGT | |
| | GGTGTTCCTGCACGTGACCTACGTGCCCGCCCAGGAGAAGAACTTCAC | |
| | CACCGCCCCCGCCATCTGCCACGACGGCAAGGCCCACTTCCCCAGGG | |
| | AGGGCGTGTTCGTGAGCAACGGCACCCACTGGTTCGTGACCCAGAGG | |
| | AACTTCTACGAGCCCCAGATCATCACCACCGACAACACCTTCGTGAGC | |
| | GGCAACTGCGACGTGGTGATCGGCATCGTGAACAACACCGTGTACGAC | |
| | CCCCTGCAGCCCGAGCTGGACAGCTTCAAGGAGGAGCTGGACAAGTA | |
| | CTTCAAGAACCACACCAGCCCCGACGTGGACCTGGGCGACATCAGCG | |
| | GCATCAACGCCAGCGTGGTGAACATCCAGAAGGAGATCGACAGGCTGA | |
| | ACGAGGTGGCCAAGAACCTGAACGAGAGCCTGATCGACCTGCAGGAG | |
| | CTGGGCAAGTACGAGCAGTACATCAAGTGGCCCTGGTACATCTGGCTG | |
| | GGCTTCATCGCCGGCCTGATCGCCATCGTGATGGTGACCATCATGCTG | |
| | TGCTGCATGACCAGCTGCTGCAGCTGCCTGAAGGGCTGCTGCAGCTGC | |
| | GGCAGCTGCTGCAAGTTCGACGAGGACGACAGCGAGCCCGTGCTGAA | |
| | GGGCGTGAAGCTGCACTACACCGGAAGCGGAGCCACGAACTTCTCTCT | |
| | GTTAAAGCAAGCAGGAGATGTTGAAGAAAACCCCGGGCCTATGGACCT | |
| | GTTCATGAGGATCTTCACCATCGGCACCGTGACCCTGAAGCAGGGCGA | |
| | GATCAAGGACGCCACCCCCAGCGACTTCGTGAGGGCCACCGCCACCA | |
| | TCCCCATCCAGGCCAGCCTGCCCTTCGGCTGGCTGATCGTGGGCGTG | |
| | GCCCTGCTGGCCGTGTTCCAGAGCGCCAGCAAGATCATCACCCTGAAG | |
| | AAGAGGTGGCAGCTGGCCCTGAGCAAGGGCGTGCACTTCGTGTGCAA | |
| | CCTGCTGCTGCTGTTCGTGACCGTGTACAGCCACCTGCTGCTGGTGGC | |
| | CGCCGGCCTGGAGGCCCCCTTCCTGTACCTGTACGCCCTGGTGTACTT | |
| | CCTGCAGAGCATCAACTTCGTGAGGATCATCATGAGGCTGTGGCTGTG | |
| | CTGGAAGTGCAGGAGCAAGAACCCCCTGCTGTACGACGCCAACTACTT | |
| | CCTGTGCTGGCACACCAACTGCTACGACTACTGCATCCCCTACAACAG | |
| | CGTGACCAGCAGCATCGTGATCACCAGCGGCGACGGCACCACCAGCC | |
| | CCATCAGCGAGCACGACTACCAGATCGGCGGCTACACCGAGAAGTGG | |
| | GAGAGCGGCGTGAAGGACTGCGTGGTGCTGCACAGCTACTTCACCAG | |
| | CGACTACTACCAGCTGTACAGCACCCAGCTGAGCACCGACACCGGCGT | |
| | GGAGCACGTGACCTTCTTCATCTACAACAAGATCGTGGACGAGCCCGA | |
| | GGAGCACGTGCAGATCCACACCATCGACGGCAGCAGCGGCGTGGTGA | |
| | ACCCCGTGATGGAGCCCATCTACGACGAGCCCACCACCACCACCAGC | |
| | GTGCCCCTGTGATGACTCGAGCTGGTACTGCATGCACGCAATGCTAGC | |
| | TGCCCCTTTCCCGTCCTGGGTACCCCGAGTCTCCCCCGACCTCGGGTC | |
| | CCAGGTATGCTCCCACCTCCACCTGCCCCACTCACCACCTCTGCTAGTT | |
| | CCAGACACCTCCCAAGCACGCAGCAATGCAGCTCAAAACGCTTAGCCT | |
| | AGCCACACCCCCACGGGAAACAGCAGTGATTAACCTTTAGCAATAAAC | |
| | GAAAGTTTAACTAAGCTATACTAACCCCAGGGTTGGTCAATTTCGTGCC | |
| | AGCCACACCCTGGAGCTAGCAAAAAAAA | |
| 21 | TAGTTATTAATAGTAATCAATTACGGGGTCATTAGTTCATAGCCCATATA | 306 |
| Ad5-Pan-CoV | TGGAGTTCCGCGTTACATAACTTACGGTAAATGGCCCGCCTGGCTGAC | |
| HexaPro- | CGCCCAACGACCCCCGCCCATTGACGTCAATAATGACGTATGTTCCCAT | |
| ORF3a | AGTAACGCCAATAGGGACTTTCCATTGACGTCAATGGGTGGAGTATTTA | |
| | CGGTAAACTGCCCACTTGGCAGTACATCAAGTGTATCATATGCCAAGTA | |
| | CGCCCCCTATTGACGTCAATGACGGTAAATGGCCCGCCTGGCATTATG | |
| | CCCAGTACATGACCTTATGGGACTTTCCTACTTGGCAGTACATCTACGT | |
| | ATTAGTCATCGCTATTACCATGGTGATGCGGTTTTGGCAGTACATCAAT | |
| | GGGCGTGGATAGCGGTTTGACTCACGGGGATTTCCAAGTCTCCACCCC | |
| | ATTGACGTCAATGGGAGTTTGTTTTGGCACCAAAATCAACGGGACTTTC | |
| | CAAAATGTCGTAACAACTCCGCCCCATTGACGCAAATGGGCGGTAGGC | |
| | GTGTACGGTGGGAGGTCTATATAAGCAGAGCTGGTTTAGTGAACCGTC | |
| | AGATCGAGAATAAACTAGTATTCTTCTGGTCCCCACAGACTCAGAGAGA | |
| | ACCCGCCACCATGTTCGTGTTCCTGGTGCTGCTGCCCCTGGTGAGCAG | |
| | CCAGTGCGTGAACCTGACCACCAGGACCCAGCTGCCCCCCGCCTACA | |
| | CCAACAGCTTCACCAGGGGCGTGTACTACCCCGACAAGGTGTTCAGGA | |
| | GCAGCGTGCTGCACAGCACCCAGGACCTGTTCCTGCCCTTCTTCAGCA | |
| | ACGTGACCTGGTTCCACGCCATCCACGTGAGCGGCACCAACGGCACCA | |
| | AGAGGTTCGACAACCCCGTGCTGCCCTTCAACGACGGCGTGTACTTCG | |
| | CCAGCACCGAGAAGAGCAACATCATCAGGGGCTGGATCTTCGGCACCA | |
| | CCCTGGACAGCAAGACCCAGAGCCTGCTGATCGTGAACAACGCCACCA | |
| | ACGTGGTGATCAAGGTGTGCGAGTTCCAGTTCTGCAACGACCCCTTCC | |
| | TGGGCGTGTACTACCACAAGAACAACAAGAGCTGGATGGAGAGCGAGT | |
| | TCAGGGTGTACAGCAGCGCCAACAACTGCACCTTCGAGTACGTGAGCC | |
| | AGCCCTTCCTGATGGACCTGGAGGGCAAGCAGGGCAACTTCAAGAACC | |
| | TGAGGGAGTTCGTGTTCAAGAACATCGACGGCTACTTCAAGATCTACAG | |
| | CAAGCACACCCCCATCAACCTGGTGAGGGACCTGCCCCAGGGCTTCAG | |
| | CGCCCTGGAGCCCCTGGTGGACCTGCCCATCGGCATCAACATCACCAG | |
| | GTTCCAGACCCTGCTGGCCCTGCACAGGAGCTACCTGACCCCCGGCG | |
| | ACAGCAGCAGCGGCTGGACCGCCGGCGCCGCCGCCTACTACGTGGGC | |
| | TACCTGCAGCCCAGGACCTTCCTGCTGAAGTACAACGAGAACGGCACC | |
| | ATCACCGACGCCGTGGACTGCGCCCTGGACCCCCTGAGCGAGACCAA | |
| | GTGCACCCTGAAGAGCTTCACCGTGGAGAAGGGCATCTACCAGACCAG | |
| | CAACTTCAGGGTGCAGCCCACCGAGAGCATCGTGAGGTTCCCCAACAT | |
| | CACCAACCTGTGCCCCTTCGGCGAGGTGTTCAACGCCACCAGGTTCGC | |
| | CAGCGTGTACGCCTGGAACAGGAAGAGGATCAGCAACTGCGTGGCCG | |
| | ACTACAGCGTGCTGTACAACAGCGCCAGCTTCAGCACCTTCAAGTGCT | |
| | ACGGCGTGAGCCCCACCAAGCTGAACGACCTGTGCTTCACCAACGTGT | |
| | ACGCCGACAGCTTCGTGATCAGGGGCGACGAGGTGAGGCAGATCGCC | |
| | CCCGGCCAGACCGGCAAGATCGCCGACTACAACTACAAGCTGCCCGA | |
| | CGACTTCACCGGCTGCGTGATCGCCTGGAACAGCAACAACCTGGACAG | |
| | CAAGGTGGGCGGCAACTACAACTACCTGTACAGGCTGTTCAGGAAGAG | |
| | CAACCTGAAGCCCTTCGAGAGGGACATCAGCACCGAGATCTACCAGGC | |
| | CGGCAGCACCCCCTGCAACGGCGTGGAGGGCTTCAACTGCTACTTCCC | |
| | CCTGCAGAGCTACGGCTTCCAGCCCACCAACGGCGTGGGCTACCAGC | |
| | CCTACAGGGTGGTGGTGCTGAGCTTCGAGCTGCTGCACGCCCCCGCC | |
| | ACCGTGTGCGGCCCCAAGAAGAGCACCAACCTGGTGAAGAACAAGTGC | |
| | GTGAACTTCAACTTCAACGGCCTGACCGGCACCGGCGTGCTGACCGAG | |
| | AGCAACAAGAAGTTCCTGCCCTTCCAGCAGTTCGGCAGGGACATCGCC | |
| | GACACCACCGACGCCGTGAGGGACCCCCAGACCCTGGAGATCCTGGA | |
| | CATCACCCCCTGCAGCTTCGGCGGCGTGAGCGTGATCACCCCCGGCA | |
| | CCAACACCAGCAACCAGGTGGCCGTGCTGTACCAGGACGTGAACTGCA | |
| | CCGAGGTGCCCGTGGCCATCCACGCCGACCAGCTGACCCCCACCTGG | |
| | AGGGTGTACAGCACCGGCAGCAACGTGTTCCAGACCAGGGCCGGCTG | |
| | CCTGATCGGCGCCGAGCACGTGAACAACAGCTACGAGTGCGACATCCC | |
| | CATCGGCGCCGGCATCTGCGCCAGCTACCAGACCCAGACCAACAGCC | |
| | CCGGCAGCGCCAGCAGCGTGGCCAGCCAGAGCATCATCGCCTACACC | |
| | ATGAGCCTGGGCGCCGAGAACAGCGTGGCCTACAGCAACAACAGCAT | |
| | CGCCATCCCCACCAACTTCACCATCAGCGTGACCACCGAGATCCTGCC | |
| | CGTGAGCATGACCAAGACCAGCGTGGACTGCACCATGTACATCTGCGG | |
| | CGACAGCACCGAGTGCAGCAACCTGCTGCTGCAGTACGGCAGCTTCTG | |
| | CACCCAGCTGAACAGGGCCCTGACCGGCATCGCCGTGGAGCAGGACA | |
| | AGAACACCCAGGAGGTGTTCGCCCAGGTGAAGCAGATCTACAAGACCC | |
| | CCCCCATCAAGGACTTCGGCGGCTTCAACTTCAGCCAGATCCTGCCCG | |
| | ACCCCAGCAAGCCCAGCAAGAGGAGCCCCATCGAGGACCTGCTGTTCA | |
| | ACAAGGTGACCCTGGCCGACGCCGGCTTCATCAAGCAGTACGGCGACT | |
| | GCCTGGGCGACATCGCCGCCAGGGACCTGATCTGCGCCCAGAAGTTC | |
| | AACGGCCTGACCGTGCTGCCCCCCCTGCTGACCGACGAGATGATCGC | |
| | CCAGTACACCAGCGCCCTGCTGGCCGGCACCATCACCAGCGGCTGGA | |
| | CCTTCGGCGCCGGCCCCGCCCTGCAGATCCCCTTCCCCATGCAGATG | |
| | GCCTACAGGTTCAACGGCATCGGCGTGACCCAGAACGTGCTGTACGAG | |
| | AACCAGAAGCTGATCGCCAACCAGTTCAACAGCGCCATCGGCAAGATC | |
| | CAGGACAGCCTGAGCAGCACCCCCAGCGCCCTGGGCAAGCTGCAGGA | |
| | CGTGGTGAACCAGAACGCCCAGGCCCTGAACACCCTGGTGAAGCAGC | |
| | TGAGCAGCAACTTCGGCGCCATCAGCAGCGTGCTGAACGACATCCTGA | |
| | GCAGGCTGGACCCCCCCGAGGCCGAGGTGCAGATCGACAGGCTGATC | |
| | ACCGGCAGGCTGCAGAGCCTGCAGACCTACGTGACCCAGCAGCTGAT | |
| | CAGGGCCGCCGAGATCAGGGCCAGCGCCAACCTGGCCGCCACCAAGA | |
| | TGAGCGAGTGCGTGCTGGGCCAGAGCAAGAGGGTGGACTTCTGCGGC | |
| | AAGGGCTACCACCTGATGAGCTTCCCCCAGAGCGCCCCCCACGGCGT | |
| | GGTGTTCCTGCACGTGACCTACGTGCCCGCCCAGGAGAAGAACTTCAC | |
| | CACCGCCCCCGCCATCTGCCACGACGGCAAGGCCCACTTCCCCAGGG | |
| | AGGGCGTGTTCGTGAGCAACGGCACCCACTGGTTCGTGACCCAGAGG | |
| | AACTTCTACGAGCCCCAGATCATCACCACCGACAACACCTTCGTGAGC | |
| | GGCAACTGCGACGTGGTGATCGGCATCGTGAACAACACCGTGTACGAC | |
| | CCCCTGCAGCCCGAGCTGGACAGCTTCAAGGAGGAGCTGGACAAGTA | |
| | CTTCAAGAACCACACCAGCCCCGACGTGGACCTGGGCGACATCAGCG | |
| | GCATCAACGCCAGCGTGGTGAACATCCAGAAGGAGATCGACAGGCTGA | |
| | ACGAGGTGGCCAAGAACCTGAACGAGAGCCTGATCGACCTGCAGGAG | |
| | CTGGGCAAGTACGAGCAGTACATCAAGTGGCCCTGGTACATCTGGCTG | |
| | GGCTTCATCGCCGGCCTGATCGCCATCGTGATGGTGACCATCATGCTG | |
| | TGCTGCATGACCAGCTGCTGCAGCTGCCTGAAGGGCTGCTGCAGCTGC | |
| | GGCAGCTGCTGCAAGTTCGACGAGGACGACAGCGAGCCCGTGCTGAA | |
| | GGGCGTGAAGCTGCACTACACCGGAAGCGGAGCCACGAACTTCTCTCT | |
| | GTTAAAGCAAGCAGGAGATGTTGAAGAAAACCCCGGGCCTATGGACCT | |
| | GTTCATGAGGATCTTCACCATCGGCACCGTGACCCTGAAGCAGGGCGA | |
| | GATCAAGGACGCCACCCCCAGCGACTTCGTGAGGGCCACCGCCACCA | |
| | TCCCCATCCAGGCCAGCCTGCCCTTCGGCTGGCTGATCGTGGGCGTG | |
| | GCCCTGCTGGCCGTGTTCCAGAGCGCCAGCAAGATCATCACCCTGAAG | |
| | AAGAGGTGGCAGCTGGCCCTGAGCAAGGGCGTGCACTTCGTGTGCAA | |
| | CCTGCTGCTGCTGTTCGTGACCGTGTACAGCCACCTGCTGCTGGTGGC | |
| | CGCCGGCCTGGAGGCCCCCTTCCTGTACCTGTACGCCCTGGTGTACTT | |
| | CCTGCAGAGCATCAACTTCGTGAGGATCATCATGAGGCTGTGGCTGTG | |
| | CTGGAAGTGCAGGAGCAAGAACCCCCTGCTGTACGACGCCAACTACTT | |
| | CCTGTGCTGGCACACCAACTGCTACGACTACTGCATCCCCTACAACAG | |
| | CGTGACCAGCAGCATCGTGATCACCAGCGGCGACGGCACCACCAGCC | |
| | CCATCAGCGAGCACGACTACCAGATCGGCGGCTACACCGAGAAGTGG | |
| | GAGAGCGGCGTGAAGGACTGCGTGGTGCTGCACAGCTACTTCACCAG | |
| | CGACTACTACCAGCTGTACAGCACCCAGCTGAGCACCGACACCGGCGT | |
| | GGAGCACGTGACCTTCTTCATCTACAACAAGATCGTGGACGAGCCCGA | |
| | GGAGCACGTGCAGATCCACACCATCGACGGCAGCAGCGGCGTGGTGA | |
| | ACCCCGTGATGGAGCCCATCTACGACGAGCCCACCACCACCACCAGC | |
| | GTGCCCCTGTGATGACTCGAGCTGGTACTGCATGCACGCAATGCTAGC | |
| | TGCCCCTTTCCCGTCCTGGGTACCCCGAGTCTCCCCCGACCTCGGGTC | |
| | CCAGGTATGCTCCCACCTCCACCTGCCCCACTCACCACCTCTGCTAGTT | |
| | CCAGACACCTCCCAAGCACGCAGCAATGCAGCTCAAAACGCTTAGCCT | |
| | AGCCACACCCCCACGGGAAACAGCAGTGATTAACCTTTAGCAATAAAC | |
| | GAAAGTTTAACTAAGCTATACTAACCCCAGGGTTGGTCAATTTCGTGCC | |
| | AGCCACACCCTGGAGCTAGCAAAAAAAA | |
| 22 | ATGTTCGTGTTCCTGGTGCTGCTGCCCCTGGTGAGCAGCCAGTGCGTG | 307 |
| Pan-CoV | AACCTGACCACCAGGACCCAGCTGCCCCCCGCCTACACCAACAGCTTC | |
| mRNA Spike- | ACCAGGGGCGTGTACTACCCCGACAAGGTGTTCAGGAGCAGCGTGCT | |
| NSP1-NSP2 | GCACAGCACCCAGGACCTGTTCCTGCCCTTCTTCAGCAACGTGACCTG | |
| | GTTCCACGCCATCCACGTGAGCGGCACCAACGGCACCAAGAGGTTCGA | |
| | CAACCCCGTGCTGCCCTTCAACGACGGCGTGTACTTCGCCAGCACCGA | |
| | GAAGAGCAACATCATCAGGGGCTGGATCTTCGGCACCACCCTGGACAG | |
| | CAAGACCCAGAGCCTGCTGATCGTGAACAACGCCACCAACGTGGTGAT | |
| | CAAGGTGTGCGAGTTCCAGTTCTGCAACGACCCCTTCCTGGGCGTGTA | |
| | CTACCACAAGAACAACAAGAGCTGGATGGAGAGCGAGTTCAGGGTGTA | |
| | CAGCAGCGCCAACAACTGCACCTTCGAGTACGTGAGCCAGCCCTTCCT | |
| | GATGGACCTGGAGGGCAAGCAGGGCAACTTCAAGAACCTGAGGGAGT | |
| | TCGTGTTCAAGAACATCGACGGCTACTTCAAGATCTACAGCAAGCACAC | |
| | CCCCATCAACCTGGTGAGGGACCTGCCCCAGGGCTTCAGCGCCCTGG | |
| | AGCCCCTGGTGGACCTGCCCATCGGCATCAACATCACCAGGTTCCAGA | |
| | CCCTGCTGGCCCTGCACAGGAGCTACCTGACCCCCGGCGACAGCAGC | |
| | AGCGGCTGGACCGCCGGCGCCGCCGCCTACTACGTGGGCTACCTGCA | |
| | GCCCAGGACCTTCCTGCTGAAGTACAACGAGAACGGCACCATCACCGA | |
| | CGCCGTGGACTGCGCCCTGGACCCCCTGAGCGAGACCAAGTGCACCC | |
| | TGAAGAGCTTCACCGTGGAGAAGGGCATCTACCAGACCAGCAACTTCA | |
| | GGGTGCAGCCCACCGAGAGCATCGTGAGGTTCCCCAACATCACCAACC | |
| | TGTGCCCCTTCGGCGAGGTGTTCAACGCCACCAGGTTCGCCAGCGTGT | |
| | ACGCCTGGAACAGGAAGAGGATCAGCAACTGCGTGGCCGACTACAGC | |
| | GTGCTGTACAACAGCGCCAGCTTCAGCACCTTCAAGTGCTACGGCGTG | |
| | AGCCCCACCAAGCTGAACGACCTGTGCTTCACCAACGTGTACGCCGAC | |
| | AGCTTCGTGATCAGGGGCGACGAGGTGAGGCAGATCGCCCCCGGCCA | |
| | GACCGGCAAGATCGCCGACTACAACTACAAGCTGCCCGACGACTTCAC | |
| | CGGCTGCGTGATCGCCTGGAACAGCAACAACCTGGACAGCAAGGTGG | |
| | GCGGCAACTACAACTACCTGTACAGGCTGTTCAGGAAGAGCAACCTGA | |
| | AGCCCTTCGAGAGGGACATCAGCACCGAGATCTACCAGGCCGGCAGC | |
| | ACCCCCTGCAACGGCGTGGAGGGCTTCAACTGCTACTTCCCCCTGCAG | |
| | AGCTACGGCTTCCAGCCCACCAACGGCGTGGGCTACCAGCCCTACAG | |
| | GGTGGTGGTGCTGAGCTTCGAGCTGCTGCACGCCCCCGCCACCGTGT | |
| | GCGGCCCCAAGAAGAGCACCAACCTGGTGAAGAACAAGTGCGTGAACT | |
| | TCAACTTCAACGGCCTGACCGGCACCGGCGTGCTGACCGAGAGCAACA | |
| | AGAAGTTCCTGCCCTTCCAGCAGTTCGGCAGGGACATCGCCGACACCA | |
| | CCGACGCCGTGAGGGACCCCCAGACCCTGGAGATCCTGGACATCACC | |
| | CCCTGCAGCTTCGGCGGCGTGAGCGTGATCACCCCCGGCACCAACAC | |
| | CAGCAACCAGGTGGCCGTGCTGTACCAGGACGTGAACTGCACCGAGG | |
| | TGCCCGTGGCCATCCACGCCGACCAGCTGACCCCCACCTGGAGGGTG | |
| | TACAGCACCGGCAGCAACGTGTTCCAGACCAGGGCCGGCTGCCTGAT | |
| | CGGCGCCGAGCACGTGAACAACAGCTACGAGTGCGACATCCCCATCG | |
| | GCGCCGGCATCTGCGCCAGCTACCAGACCCAGACCAACAGCCCCAGG | |
| | AGGGCCAGGAGCGTGGCCAGCCAGAGCATCATCGCCTACACCATGAG | |
| | CCTGGGCGCCGAGAACAGCGTGGCCTACAGCAACAACAGCATCGCCA | |
| | TCCCCACCAACTTCACCATCAGCGTGACCACCGAGATCCTGCCCGTGA | |
| | GCATGACCAAGACCAGCGTGGACTGCACCATGTACATCTGCGGCGACA | |
| | GCACCGAGTGCAGCAACCTGCTGCTGCAGTACGGCAGCTTCTGCACCC | |
| | AGCTGAACAGGGCCCTGACCGGCATCGCCGTGGAGCAGGACAAGAAC | |
| | ACCCAGGAGGTGTTCGCCCAGGTGAAGCAGATCTACAAGACCCCCCCC | |
| | ATCAAGGACTTCGGCGGCTTCAACTTCAGCCAGATCCTGCCCGACCCC | |
| | AGCAAGCCCAGCAAGAGGAGCTTCATCGAGGACCTGCTGTTCAACAAG | |
| | GTGACCCTGGCCGACGCCGGCTTCATCAAGCAGTACGGCGACTGCCT | |
| | GGGCGACATCGCCGCCAGGGACCTGATCTGCGCCCAGAAGTTCAACG | |
| | GCCTGACCGTGCTGCCCCCCCTGCTGACCGACGAGATGATCGCCCAG | |
| | TACACCAGCGCCCTGCTGGCCGGCACCATCACCAGCGGCTGGACCTT | |
| | CGGCGCCGGCGCCGCCCTGCAGATCCCCTTCGCCATGCAGATGGCCT | |
| | ACAGGTTCAACGGCATCGGCGTGACCCAGAACGTGCTGTACGAGAACC | |
| | AGAAGCTGATCGCCAACCAGTTCAACAGCGCCATCGGCAAGATCCAGG | |
| | ACAGCCTGAGCAGCACCGCCAGCGCCCTGGGCAAGCTGCAGGACGTG | |
| | GTGAACCAGAACGCCCAGGCCCTGAACACCCTGGTGAAGCAGCTGAG | |
| | CAGCAACTTCGGCGCCATCAGCAGCGTGCTGAACGACATCCTGAGCAG | |
| | GCTGGACCCCCCCGAGGCCGAGGTGCAGATCGACAGGCTGATCACCG | |
| | GCAGGCTGCAGAGCCTGCAGACCTACGTGACCCAGCAGCTGATCAGG | |
| | GCCGCCGAGATCAGGGCCAGCGCCAACCTGGCCGCCACCAAGATGAG | |
| | CGAGTGCGTGCTGGGCCAGAGCAAGAGGGTGGACTTCTGCGGCAAGG | |
| | GCTACCACCTGATGAGCTTCCCCCAGAGCGCCCCCCACGGCGTGGTG | |
| | TTCCTGCACGTGACCTACGTGCCCGCCCAGGAGAAGAACTTCACCACC | |
| | GCCCCCGCCATCTGCCACGACGGCAAGGCCCACTTCCCCAGGGAGGG | |
| | CGTGTTCGTGAGCAACGGCACCCACTGGTTCGTGACCCAGAGGAACTT | |
| | CTACGAGCCCCAGATCATCACCACCGACAACACCTTCGTGAGCGGCAA | |
| | CTGCGACGTGGTGATCGGCATCGTGAACAACACCGTGTACGACCCCCT | |
| | GCAGCCCGAGCTGGACAGCTTCAAGGAGGAGCTGGACAAGTACTTCAA | |
| | GAACCACACCAGCCCCGACGTGGACCTGGGCGACATCAGCGGCATCA | |
| | ACGCCAGCGTGGTGAACATCCAGAAGGAGATCGACAGGCTGAACGAG | |
| | GTGGCCAAGAACCTGAACGAGAGCCTGATCGACCTGCAGGAGCTGGG | |
| | CAAGTACGAGCAGTACATCAAGTGGCCCTGGTACATCTGGCTGGGCTT | |
| | CATCGCCGGCCTGATCGCCATCGTGATGGTGACCATCATGCTGTGCTG | |
| | CATGACCAGCTGCTGCAGCTGCCTGAAGGGCTGCTGCAGCTGCGGCA | |
| | GCTGCTGCAAGTTCGACGAGGACGACAGCGAGCCCGTGCTGAAGGGC | |
| | GTGAAGCTGCACTACACCGGAAGCGGAGCCACGAACTTCTCTCTGTTA | |
| | AAGCAAGCAGGAGATGTTGAAGAAAACCCCGGGCCTATGGAGAGCCTG | |
| | GTGCCCGGCTTCAACGAGAAGACCCACGTGCAGCTGAGCCTGCCCGT | |
| | GCTGCAGGTGAGGGACGTGCTGGTGAGGGGCTTCGGCGACAGCGTGG | |
| | AGGAGGTGCTGAGCGAGGCCAGGCAGCACCTGAAGGACGGCACCTGC | |
| | GGCCTGGTGGAGGTGGAGAAGGGCGTGCTGCCCCAGCTGGAGCAGC | |
| | CCTACGTGTTCATCAAGAGGAGCGACGCCAGGACCGCCCCCCACGGC | |
| | CACGTGATGGTGGAGCTGGTGGCCGAGCTGGAGGGCATCCAGTACGG | |
| | CAGGAGCGGCGAGACCCTGGGCGTGCTGGTGCCCCACGTGGGCGAG | |
| | ATCCCCGTGGCCTACAGGAAGGTGCTGCTGAGGAAGAACGGCAACAA | |
| | GGGCGCCGGCGGCCACAGCTACGGCGCCGACCTGAAGAGCTTCGACC | |
| | TGGGCGACGAGCTGGGCACCGACCCCTACGAGGACTTCCAGGAGAAC | |
| | TGGAACACCAAGCACAGCAGCGGCGTGACCAGGGAGCTGATGAGGGA | |
| | GCTGAACGGCGGCGGAAGCGGAGAGGGCAGGGGAAGTCTTCTAACAT | |
| | GCGGGGACGTGGAGGAAAATCCCGGCCCCGCCTACACCAGGTACGTG | |
| | GACAACAACTTCTGCGGCCCCGACGGCTACCCCCTGGAGTGCATCAAG | |
| | GACCTGCTGGCCAGGGCCGGCAAGGCCAGCTGCACCCTGAGCGAGCA | |
| | GCTGGACTTCATCGACACCAAGAGGGGCGTGTACTGCTGCAGGGAGC | |
| | ACGAGCACGAGATCGCCTGGTACACCGAGAGGAGCGAGAAGAGCTAC | |
| | GAGCTGCAGACCCCCTTCGAGATCAAGCTGGCCAAGAAGTTCGACACC | |
| | TTCAACGGCGAGTGCCCCAACTTCGTGTTCCCCCTGAACAGCATCATCA | |
| | AGACCATCCAGCCCAGGGTGGAGAAGAAGAAGCTGGACGGCTTCATG | |
| | GGCAGGATCAGGAGCGTGTACCCCGTGGCCAGCCCCAACGAGTGCAA | |
| | CCAGATGTGCCTGAGCACCCTGATGAAGTGCGACCACTGCGGCGAGA | |
| | CCAGCTGGCAGACCGGCGACTTCGTGAAGGCCACCTGCGAGTTCTGC | |
| | GGCACCGAGAACCTGACCAAGGAGGGCGCCACCACCTGCGGCTACCT | |
| | GCCCCAGAACGCCGTGGTGAAGATCTACTGCCCCGCCTGCCACAACAG | |
| | CGAGGTGGGCCCCGAGCACAGCCTGGCCGAGTACCACAACGAGAGCG | |
| | GCCTGAAGACCATCCTGAGGAAGGGCGGCAGGACCATCGCCTTCGGC | |
| | GGCTGCGTGTTCAGCTACGTGGGCTGCCACAACAAGTGCGCCTACTGG | |
| | GTGCCCAGGGCCAGCGCCAACATCGGCTGCAACCACACCGGCGTGGT | |
| | GGGCGAGGGCAGCGAGGGCCTGAACGACAACCTGCTGGAGATCCTGC | |
| | AGAAGGAGAAGGTGAACATCAACATCGTGGGCGACTTCAAGCTGAACG | |
| | AGGAGATCGCCATCATCCTGGCCAGCTTCAGCGCCAGCACCAGCGCCT | |
| | TCGTGGAGACCGTGAAGGGCCTGGACTACAAGGCCTTCAAGCAGATCG | |
| | TGGAGAGCTGCGGCAACTTCAAGGTGACCAAGGGCAAGGCCAAGAAG | |
| | GGCGCCTGGAACATCGGCGAGCAGAAGAGCATCCTGAGCCCCCTGTA | |
| | CGCCTTCGCCAGCGAGGCCGCCAGGGTGGTGAGGAGCATCTTCAGCA | |
| | GGACCCTGGAGACCGCCCAGAACAGCGTGAGGGTGCTGCAGAAGGCC | |
| | GCCATCACCATCCTGGACGGCATCAGCCAGTACAGCCTGAGGCTGATC | |
| | GACGCCATGATGTTCACCAGCGACCTGGCCACCAACAACCTGGTGGTG | |
| | ATGGCCTACATCACCGGCGGCGTGGTGCAGCTGACCAGCCAGTGGCT | |
| | GACCAACATCTTCGGCACCGTGTACGAGAAGCTGAAGCCCGTGCTGGA | |
| | CTGGCTGGAGGAGAAGTTCAAGGAGGGCGTGGAGTTCCTGAGGGACG | |
| | GCTGGGAGATCGTGAAGTTCATCAGCACCTGCGCCTGCGAGATCGTGG | |
| | GCGGCCAGATCGTGACCTGCGCCAAGGAGATCAAGGAGAGCGTGCAG | |
| | ACCTTCTTCAAGCTGGTGAACAAGTTCCTGGCCCTGTGCGCCGACAGC | |
| | ATCATCATCGGCGGCGCCAAGCTGAAGGCCCTGAACCTGGGCGAGAC | |
| | CTTCGTGACCCACAGCAAGGGCCTGTACAGGAAGTGCGTGAAGAGCAG | |
| | GGAGGAGACCGGCCTGCTGATGCCCCTGAAGGCCCCCAAGGAGATCA | |
| | TCTTCCTGGAGGGCGAGACCCTGCCCACCGAGGTGCTGACCGAGGAG | |
| | GTGGTGCTGAAGACCGGCGACCTGCAGCCCCTGGAGCAGCCCACCAG | |
| | CGAGGCCGTGGAGGCCCCCCTGGTGGGCACCCCCGTGTGCATCAACG | |
| | GCCTGATGCTGCTGGAGATCAAGGACACCGAGAAGTACTGCGCCCTGG | |
| | CCCCCAACATGATGGTGACCAACAACACCTTCACCCTGAAGGGCGGCT | |
| | GATGA | |
| 23 | ATGTTCGTGTTCCTGGTGCTGCTGCCCCTGGTGAGCAGCCAGTGCGTG | 308 |
| Pan-CoV | AACCTGACCACCAGGACCCAGCTGCCCCCCGCCTACACCAACAGCTTC | |
| mRNA | ACCAGGGGCGTGTACTACCCCGACAAGGTGTTCAGGAGCAGCGTGCT | |
| HexaPro- | GCACAGCACCCAGGACCTGTTCCTGCCCTTCTTCAGCAACGTGACCTG | |
| NSP1-NSP2 | GTTCCACGCCATCCACGTGAGCGGCACCAACGGCACCAAGAGGTTCGA | |
| | CAACCCCGTGCTGCCCTTCAACGACGGCGTGTACTTCGCCAGCACCGA | |
| | GAAGAGCAACATCATCAGGGGCTGGATCTTCGGCACCACCCTGGACAG | |
| | CAAGACCCAGAGCCTGCTGATCGTGAACAACGCCACCAACGTGGTGAT | |
| | CAAGGTGTGCGAGTTCCAGTTCTGCAACGACCCCTTCCTGGGCGTGTA | |
| | CTACCACAAGAACAACAAGAGCTGGATGGAGAGCGAGTTCAGGGTGTA | |
| | CAGCAGCGCCAACAACTGCACCTTCGAGTACGTGAGCCAGCCCTTCCT | |
| | GATGGACCTGGAGGGCAAGCAGGGCAACTTCAAGAACCTGAGGGAGT | |
| | TCGTGTTCAAGAACATCGACGGCTACTTCAAGATCTACAGCAAGCACAC | |
| | CCCCATCAACCTGGTGAGGGACCTGCCCCAGGGCTTCAGCGCCCTGG | |
| | AGCCCCTGGTGGACCTGCCCATCGGCATCAACATCACCAGGTTCCAGA | |
| | CCCTGCTGGCCCTGCACAGGAGCTACCTGACCCCCGGCGACAGCAGC | |
| | AGCGGCTGGACCGCCGGCGCCGCCGCCTACTACGTGGGCTACCTGCA | |
| | GCCCAGGACCTTCCTGCTGAAGTACAACGAGAACGGCACCATCACCGA | |
| | CGCCGTGGACTGCGCCCTGGACCCCCTGAGCGAGACCAAGTGCACCC | |
| | TGAAGAGCTTCACCGTGGAGAAGGGCATCTACCAGACCAGCAACTTCA | |
| | GGGTGCAGCCCACCGAGAGCATCGTGAGGTTCCCCAACATCACCAACC | |
| | TGTGCCCCTTCGGCGAGGTGTTCAACGCCACCAGGTTCGCCAGCGTGT | |
| | ACGCCTGGAACAGGAAGAGGATCAGCAACTGCGTGGCCGACTACAGC | |
| | GTGCTGTACAACAGCGCCAGCTTCAGCACCTTCAAGTGCTACGGCGTG | |
| | AGCCCCACCAAGCTGAACGACCTGTGCTTCACCAACGTGTACGCCGAC | |
| | AGCTTCGTGATCAGGGGCGACGAGGTGAGGCAGATCGCCCCCGGCCA | |
| | GACCGGCAAGATCGCCGACTACAACTACAAGCTGCCCGACGACTTCAC | |
| | CGGCTGCGTGATCGCCTGGAACAGCAACAACCTGGACAGCAAGGTGG | |
| | GCGGCAACTACAACTACCTGTACAGGCTGTTCAGGAAGAGCAACCTGA | |
| | AGCCCTTCGAGAGGGACATCAGCACCGAGATCTACCAGGCCGGCAGC | |
| | ACCCCCTGCAACGGCGTGGAGGGCTTCAACTGCTACTTCCCCCTGCAG | |
| | AGCTACGGCTTCCAGCCCACCAACGGCGTGGGCTACCAGCCCTACAG | |
| | GGTGGTGGTGCTGAGCTTCGAGCTGCTGCACGCCCCCGCCACCGTGT | |
| | GCGGCCCCAAGAAGAGCACCAACCTGGTGAAGAACAAGTGCGTGAACT | |
| | TCAACTTCAACGGCCTGACCGGCACCGGCGTGCTGACCGAGAGCAACA | |
| | AGAAGTTCCTGCCCTTCCAGCAGTTCGGCAGGGACATCGCCGACACCA | |
| | CCGACGCCGTGAGGGACCCCCAGACCCTGGAGATCCTGGACATCACC | |
| | CCCTGCAGCTTCGGCGGCGTGAGCGTGATCACCCCCGGCACCAACAC | |
| | CAGCAACCAGGTGGCCGTGCTGTACCAGGACGTGAACTGCACCGAGG | |
| | TGCCCGTGGCCATCCACGCCGACCAGCTGACCCCCACCTGGAGGGTG | |
| | TACAGCACCGGCAGCAACGTGTTCCAGACCAGGGCCGGCTGCCTGAT | |
| | CGGCGCCGAGCACGTGAACAACAGCTACGAGTGCGACATCCCCATCG | |
| | GCGCCGGCATCTGCGCCAGCTACCAGACCCAGACCAACAGCCCCGGC | |
| | AGCGCCAGCAGCGTGGCCAGCCAGAGCATCATCGCCTACACCATGAG | |
| | CCTGGGCGCCGAGAACAGCGTGGCCTACAGCAACAACAGCATCGCCA | |
| | TCCCCACCAACTTCACCATCAGCGTGACCACCGAGATCCTGCCCGTGA | |
| | GCATGACCAAGACCAGCGTGGACTGCACCATGTACATCTGCGGCGACA | |
| | GCACCGAGTGCAGCAACCTGCTGCTGCAGTACGGCAGCTTCTGCACCC | |
| | AGCTGAACAGGGCCCTGACCGGCATCGCCGTGGAGCAGGACAAGAAC | |
| | ACCCAGGAGGTGTTCGCCCAGGTGAAGCAGATCTACAAGACCCCCCCC | |
| | ATCAAGGACTTCGGCGGCTTCAACTTCAGCCAGATCCTGCCCGACCCC | |
| | AGCAAGCCCAGCAAGAGGAGCCCCATCGAGGACCTGCTGTTCAACAAG | |
| | GTGACCCTGGCCGACGCCGGCTTCATCAAGCAGTACGGCGACTGCCT | |
| | GGGCGACATCGCCGCCAGGGACCTGATCTGCGCCCAGAAGTTCAACG | |
| | GCCTGACCGTGCTGCCCCCCCTGCTGACCGACGAGATGATCGCCCAG | |
| | TACACCAGCGCCCTGCTGGCCGGCACCATCACCAGCGGCTGGACCTT | |
| | CGGCGCCGGCCCCGCCCTGCAGATCCCCTTCCCCATGCAGATGGCCT | |
| | ACAGGTTCAACGGCATCGGCGTGACCCAGAACGTGCTGTACGAGAACC | |
| | AGAAGCTGATCGCCAACCAGTTCAACAGCGCCATCGGCAAGATCCAGG | |
| | ACAGCCTGAGCAGCACCCCCAGCGCCCTGGGCAAGCTGCAGGACGTG | |
| | GTGAACCAGAACGCCCAGGCCCTGAACACCCTGGTGAAGCAGCTGAG | |
| | CAGCAACTTCGGCGCCATCAGCAGCGTGCTGAACGACATCCTGAGCAG | |
| | GCTGGACCCCCCCGAGGCCGAGGTGCAGATCGACAGGCTGATCACCG | |
| | GCAGGCTGCAGAGCCTGCAGACCTACGTGACCCAGCAGCTGATCAGG | |
| | GCCGCCGAGATCAGGGCCAGCGCCAACCTGGCCGCCACCAAGATGAG | |
| | CGAGTGCGTGCTGGGCCAGAGCAAGAGGGTGGACTTCTGCGGCAAGG | |
| | GCTACCACCTGATGAGCTTCCCCCAGAGCGCCCCCCACGGCGTGGTG | |
| | TTCCTGCACGTGACCTACGTGCCCGCCCAGGAGAAGAACTTCACCACC | |
| | GCCCCCGCCATCTGCCACGACGGCAAGGCCCACTTCCCCAGGGAGGG | |
| | CGTGTTCGTGAGCAACGGCACCCACTGGTTCGTGACCCAGAGGAACTT | |
| | CTACGAGCCCCAGATCATCACCACCGACAACACCTTCGTGAGCGGCAA | |
| | CTGCGACGTGGTGATCGGCATCGTGAACAACACCGTGTACGACCCCCT | |
| | GCAGCCCGAGCTGGACAGCTTCAAGGAGGAGCTGGACAAGTACTTCAA | |
| | GAACCACACCAGCCCCGACGTGGACCTGGGCGACATCAGCGGCATCA | |
| | ACGCCAGCGTGGTGAACATCCAGAAGGAGATCGACAGGCTGAACGAG | |
| | GTGGCCAAGAACCTGAACGAGAGCCTGATCGACCTGCAGGAGCTGGG | |
| | CAAGTACGAGCAGTACATCAAGTGGCCCTGGTACATCTGGCTGGGCTT | |
| | CATCGCCGGCCTGATCGCCATCGTGATGGTGACCATCATGCTGTGCTG | |
| | CATGACCAGCTGCTGCAGCTGCCTGAAGGGCTGCTGCAGCTGCGGCA | |
| | GCTGCTGCAAGTTCGACGAGGACGACAGCGAGCCCGTGCTGAAGGGC | |
| | GTGAAGCTGCACTACACCGGAAGCGGAGCCACGAACTTCTCTCTGTTA | |
| | AAGCAAGCAGGAGATGTTGAAGAAAACCCCGGGCCTATGGAGAGCCTG | |
| | GTGCCCGGCTTCAACGAGAAGACCCACGTGCAGCTGAGCCTGCCCGT | |
| | GCTGCAGGTGAGGGACGTGCTGGTGAGGGGCTTCGGCGACAGCGTGG | |
| | AGGAGGTGCTGAGCGAGGCCAGGCAGCACCTGAAGGACGGCACCTGC | |
| | GGCCTGGTGGAGGTGGAGAAGGGCGTGCTGCCCCAGCTGGAGCAGC | |
| | CCTACGTGTTCATCAAGAGGAGCGACGCCAGGACCGCCCCCCACGGC | |
| | CACGTGATGGTGGAGCTGGTGGCCGAGCTGGAGGGCATCCAGTACGG | |
| | CAGGAGCGGCGAGACCCTGGGCGTGCTGGTGCCCCACGTGGGCGAG | |
| | ATCCCCGTGGCCTACAGGAAGGTGCTGCTGAGGAAGAACGGCAACAA | |
| | GGGCGCCGGCGGCCACAGCTACGGCGCCGACCTGAAGAGCTTCGACC | |
| | TGGGCGACGAGCTGGGCACCGACCCCTACGAGGACTTCCAGGAGAAC | |
| | TGGAACACCAAGCACAGCAGCGGCGTGACCAGGGAGCTGATGAGGGA | |
| | GCTGAACGGCGGCGGAAGCGGAGAGGGCAGGGGAAGTCTTCTAACAT | |
| | GCGGGGACGTGGAGGAAAATCCCGGCCCCGCCTACACCAGGTACGTG | |
| | GACAACAACTTCTGCGGCCCCGACGGCTACCCCCTGGAGTGCATCAAG | |
| | GACCTGCTGGCCAGGGCCGGCAAGGCCAGCTGCACCCTGAGCGAGCA | |
| | GCTGGACTTCATCGACACCAAGAGGGGCGTGTACTGCTGCAGGGAGC | |
| | ACGAGCACGAGATCGCCTGGTACACCGAGAGGAGCGAGAAGAGCTAC | |
| | GAGCTGCAGACCCCCTTCGAGATCAAGCTGGCCAAGAAGTTCGACACC | |
| | TTCAACGGCGAGTGCCCCAACTTCGTGTTCCCCCTGAACAGCATCATCA | |
| | AGACCATCCAGCCCAGGGTGGAGAAGAAGAAGCTGGACGGCTTCATG | |
| | GGCAGGATCAGGAGCGTGTACCCCGTGGCCAGCCCCAACGAGTGCAA | |
| | CCAGATGTGCCTGAGCACCCTGATGAAGTGCGACCACTGCGGCGAGA | |
| | CCAGCTGGCAGACCGGCGACTTCGTGAAGGCCACCTGCGAGTTCTGC | |
| | GGCACCGAGAACCTGACCAAGGAGGGCGCCACCACCTGCGGCTACCT | |
| | GCCCCAGAACGCCGTGGTGAAGATCTACTGCCCCGCCTGCCACAACAG | |
| | CGAGGTGGGCCCCGAGCACAGCCTGGCCGAGTACCACAACGAGAGCG | |
| | GCCTGAAGACCATCCTGAGGAAGGGCGGCAGGACCATCGCCTTCGGC | |
| | GGCTGCGTGTTCAGCTACGTGGGCTGCCACAACAAGTGCGCCTACTGG | |
| | GTGCCCAGGGCCAGCGCCAACATCGGCTGCAACCACACCGGCGTGGT | |
| | GGGCGAGGGCAGCGAGGGCCTGAACGACAACCTGCTGGAGATCCTGC | |
| | AGAAGGAGAAGGTGAACATCAACATCGTGGGCGACTTCAAGCTGAACG | |
| | AGGAGATCGCCATCATCCTGGCCAGCTTCAGCGCCAGCACCAGCGCCT | |
| | TCGTGGAGACCGTGAAGGGCCTGGACTACAAGGCCTTCAAGCAGATCG | |
| | TGGAGAGCTGCGGCAACTTCAAGGTGACCAAGGGCAAGGCCAAGAAG | |
| | GGCGCCTGGAACATCGGCGAGCAGAAGAGCATCCTGAGCCCCCTGTA | |
| | CGCCTTCGCCAGCGAGGCCGCCAGGGTGGTGAGGAGCATCTTCAGCA | |
| | GGACCCTGGAGACCGCCCAGAACAGCGTGAGGGTGCTGCAGAAGGCC | |
| | GCCATCACCATCCTGGACGGCATCAGCCAGTACAGCCTGAGGCTGATC | |
| | GACGCCATGATGTTCACCAGCGACCTGGCCACCAACAACCTGGTGGTG | |
| | ATGGCCTACATCACCGGCGGCGTGGTGCAGCTGACCAGCCAGTGGCT | |
| | GACCAACATCTTCGGCACCGTGTACGAGAAGCTGAAGCCCGTGCTGGA | |
| | CTGGCTGGAGGAGAAGTTCAAGGAGGGCGTGGAGTTCCTGAGGGACG | |
| | GCTGGGAGATCGTGAAGTTCATCAGCACCTGCGCCTGCGAGATCGTGG | |
| | GCGGCCAGATCGTGACCTGCGCCAAGGAGATCAAGGAGAGCGTGCAG | |
| | ACCTTCTTCAAGCTGGTGAACAAGTTCCTGGCCCTGTGCGCCGACAGC | |
| | ATCATCATCGGCGGCGCCAAGCTGAAGGCCCTGAACCTGGGCGAGAC | |
| | CTTCGTGACCCACAGCAAGGGCCTGTACAGGAAGTGCGTGAAGAGCAG | |
| | GGAGGAGACCGGCCTGCTGATGCCCCTGAAGGCCCCCAAGGAGATCA | |
| | TCTTCCTGGAGGGCGAGACCCTGCCCACCGAGGTGCTGACCGAGGAG | |
| | GTGGTGCTGAAGACCGGCGACCTGCAGCCCCTGGAGCAGCCCACCAG | |
| | CGAGGCCGTGGAGGCCCCCCTGGTGGGCACCCCCGTGTGCATCAACG | |
| | GCCTGATGCTGCTGGAGATCAAGGACACCGAGAAGTACTGCGCCCTGG | |
| | CCCCCAACATGATGGTGACCAACAACACCTTCACCCTGAAGGGCGGCT | |
| | GATGA | |
| 24 | ATGTTCGTGTTCCTGGTGCTGCTGCCCCTGGTGAGCAGCCAGTGCGTG | 309 |
| Pan-CoV | AACCTGACCACCAGGACCCAGCTGCCCCCCGCCTACACCAACAGCTTC | |
| mRNA Spike- | ACCAGGGGCGTGTACTACCCCGACAAGGTGTTCAGGAGCAGCGTGCT | |
| NSP6-NSP7- | GCACAGCACCCAGGACCTGTTCCTGCCCTTCTTCAGCAACGTGACCTG | |
| NSP8 | GTTCCACGCCATCCACGTGAGCGGCACCAACGGCACCAAGAGGTTCGA | |
| | CAACCCCGTGCTGCCCTTCAACGACGGCGTGTACTTCGCCAGCACCGA | |
| | GAAGAGCAACATCATCAGGGGCTGGATCTTCGGCACCACCCTGGACAG | |
| | CAAGACCCAGAGCCTGCTGATCGTGAACAACGCCACCAACGTGGTGAT | |
| | CAAGGTGTGCGAGTTCCAGTTCTGCAACGACCCCTTCCTGGGCGTGTA | |
| | CTACCACAAGAACAACAAGAGCTGGATGGAGAGCGAGTTCAGGGTGTA | |
| | CAGCAGCGCCAACAACTGCACCTTCGAGTACGTGAGCCAGCCCTTCCT | |
| | GATGGACCTGGAGGGCAAGCAGGGCAACTTCAAGAACCTGAGGGAGT | |
| | TCGTGTTCAAGAACATCGACGGCTACTTCAAGATCTACAGCAAGCACAC | |
| | CCCCATCAACCTGGTGAGGGACCTGCCCCAGGGCTTCAGCGCCCTGG | |
| | AGCCCCTGGTGGACCTGCCCATCGGCATCAACATCACCAGGTTCCAGA | |
| | CCCTGCTGGCCCTGCACAGGAGCTACCTGACCCCCGGCGACAGCAGC | |
| | AGCGGCTGGACCGCCGGCGCCGCCGCCTACTACGTGGGCTACCTGCA | |
| | GCCCAGGACCTTCCTGCTGAAGTACAACGAGAACGGCACCATCACCGA | |
| | CGCCGTGGACTGCGCCCTGGACCCCCTGAGCGAGACCAAGTGCACCC | |
| | TGAAGAGCTTCACCGTGGAGAAGGGCATCTACCAGACCAGCAACTTCA | |
| | GGGTGCAGCCCACCGAGAGCATCGTGAGGTTCCCCAACATCACCAACC | |
| | TGTGCCCCTTCGGCGAGGTGTTCAACGCCACCAGGTTCGCCAGCGTGT | |
| | ACGCCTGGAACAGGAAGAGGATCAGCAACTGCGTGGCCGACTACAGC | |
| | GTGCTGTACAACAGCGCCAGCTTCAGCACCTTCAAGTGCTACGGCGTG | |
| | AGCCCCACCAAGCTGAACGACCTGTGCTTCACCAACGTGTACGCCGAC | |
| | AGCTTCGTGATCAGGGGCGACGAGGTGAGGCAGATCGCCCCCGGCCA | |
| | GACCGGCAAGATCGCCGACTACAACTACAAGCTGCCCGACGACTTCAC | |
| | CGGCTGCGTGATCGCCTGGAACAGCAACAACCTGGACAGCAAGGTGG | |
| | GCGGCAACTACAACTACCTGTACAGGCTGTTCAGGAAGAGCAACCTGA | |
| | AGCCCTTCGAGAGGGACATCAGCACCGAGATCTACCAGGCCGGCAGC | |
| | ACCCCCTGCAACGGCGTGGAGGGCTTCAACTGCTACTTCCCCCTGCAG | |
| | AGCTACGGCTTCCAGCCCACCAACGGCGTGGGCTACCAGCCCTACAG | |
| | GGTGGTGGTGCTGAGCTTCGAGCTGCTGCACGCCCCCGCCACCGTGT | |
| | GCGGCCCCAAGAAGAGCACCAACCTGGTGAAGAACAAGTGCGTGAACT | |
| | TCAACTTCAACGGCCTGACCGGCACCGGCGTGCTGACCGAGAGCAACA | |
| | AGAAGTTCCTGCCCTTCCAGCAGTTCGGCAGGGACATCGCCGACACCA | |
| | CCGACGCCGTGAGGGACCCCCAGACCCTGGAGATCCTGGACATCACC | |
| | CCCTGCAGCTTCGGCGGCGTGAGCGTGATCACCCCCGGCACCAACAC | |
| | CAGCAACCAGGTGGCCGTGCTGTACCAGGACGTGAACTGCACCGAGG | |
| | TGCCCGTGGCCATCCACGCCGACCAGCTGACCCCCACCTGGAGGGTG | |
| | TACAGCACCGGCAGCAACGTGTTCCAGACCAGGGCCGGCTGCCTGAT | |
| | CGGCGCCGAGCACGTGAACAACAGCTACGAGTGCGACATCCCCATCG | |
| | GCGCCGGCATCTGCGCCAGCTACCAGACCCAGACCAACAGCCCCAGG | |
| | AGGGCCAGGAGCGTGGCCAGCCAGAGCATCATCGCCTACACCATGAG | |
| | CCTGGGCGCCGAGAACAGCGTGGCCTACAGCAACAACAGCATCGCCA | |
| | TCCCCACCAACTTCACCATCAGCGTGACCACCGAGATCCTGCCCGTGA | |
| | GCATGACCAAGACCAGCGTGGACTGCACCATGTACATCTGCGGCGACA | |
| | GCACCGAGTGCAGCAACCTGCTGCTGCAGTACGGCAGCTTCTGCACCC | |
| | AGCTGAACAGGGCCCTGACCGGCATCGCCGTGGAGCAGGACAAGAAC | |
| | ACCCAGGAGGTGTTCGCCCAGGTGAAGCAGATCTACAAGACCCCCCCC | |
| | ATCAAGGACTTCGGCGGCTTCAACTTCAGCCAGATCCTGCCCGACCCC | |
| | AGCAAGCCCAGCAAGAGGAGCTTCATCGAGGACCTGCTGTTCAACAAG | |
| | GTGACCCTGGCCGACGCCGGCTTCATCAAGCAGTACGGCGACTGCCT | |
| | GGGCGACATCGCCGCCAGGGACCTGATCTGCGCCCAGAAGTTCAACG | |
| | GCCTGACCGTGCTGCCCCCCCTGCTGACCGACGAGATGATCGCCCAG | |
| | TACACCAGCGCCCTGCTGGCCGGCACCATCACCAGCGGCTGGACCTT | |
| | CGGCGCCGGCGCCGCCCTGCAGATCCCCTTCGCCATGCAGATGGCCT | |
| | ACAGGTTCAACGGCATCGGCGTGACCCAGAACGTGCTGTACGAGAACC | |
| | AGAAGCTGATCGCCAACCAGTTCAACAGCGCCATCGGCAAGATCCAGG | |
| | ACAGCCTGAGCAGCACCGCCAGCGCCCTGGGCAAGCTGCAGGACGTG | |
| | GTGAACCAGAACGCCCAGGCCCTGAACACCCTGGTGAAGCAGCTGAG | |
| | CAGCAACTTCGGCGCCATCAGCAGCGTGCTGAACGACATCCTGAGCAG | |
| | GCTGGACCCCCCCGAGGCCGAGGTGCAGATCGACAGGCTGATCACCG | |
| | GCAGGCTGCAGAGCCTGCAGACCTACGTGACCCAGCAGCTGATCAGG | |
| | GCCGCCGAGATCAGGGCCAGCGCCAACCTGGCCGCCACCAAGATGAG | |
| | CGAGTGCGTGCTGGGCCAGAGCAAGAGGGTGGACTTCTGCGGCAAGG | |
| | GCTACCACCTGATGAGCTTCCCCCAGAGCGCCCCCCACGGCGTGGTG | |
| | TTCCTGCACGTGACCTACGTGCCCGCCCAGGAGAAGAACTTCACCACC | |
| | GCCCCCGCCATCTGCCACGACGGCAAGGCCCACTTCCCCAGGGAGGG | |
| | CGTGTTCGTGAGCAACGGCACCCACTGGTTCGTGACCCAGAGGAACTT | |
| | CTACGAGCCCCAGATCATCACCACCGACAACACCTTCGTGAGCGGCAA | |
| | CTGCGACGTGGTGATCGGCATCGTGAACAACACCGTGTACGACCCCCT | |
| | GCAGCCCGAGCTGGACAGCTTCAAGGAGGAGCTGGACAAGTACTTCAA | |
| | GAACCACACCAGCCCCGACGTGGACCTGGGCGACATCAGCGGCATCA | |
| | ACGCCAGCGTGGTGAACATCCAGAAGGAGATCGACAGGCTGAACGAG | |
| | GTGGCCAAGAACCTGAACGAGAGCCTGATCGACCTGCAGGAGCTGGG | |
| | CAAGTACGAGCAGTACATCAAGTGGCCCTGGTACATCTGGCTGGGCTT | |
| | CATCGCCGGCCTGATCGCCATCGTGATGGTGACCATCATGCTGTGCTG | |
| | CATGACCAGCTGCTGCAGCTGCCTGAAGGGCTGCTGCAGCTGCGGCA | |
| | GCTGCTGCAAGTTCGACGAGGACGACAGCGAGCCCGTGCTGAAGGGC | |
| | GTGAAGCTGCACTACACCGGAAGCGGAGCCACGAACTTCTCTCTGTTA | |
| | AAGCAAGCAGGAGATGTTGAAGAAAACCCCGGGCCTAGCGCCGTGAA | |
| | GAGGACCATCAAGGGCACCCACCACTGGCTGCTGCTGACCATCCTGAC | |
| | CAGCCTGCTGGTGCTGGTGCAGAGCACCCAGTGGAGCCTGTTCTTCTT | |
| | CCTGTACGAGAACGCCTTCCTGCCCTTCGCCATGGGCATCATCGCCAT | |
| | GAGCGCCTTCGCCATGATGTTCGTGAAGCACAAGCACGCCTTCCTGTG | |
| | CCTGTTCCTGCTGCCCAGCCTGGCCACCGTGGCCTACTTCAACATGGT | |
| | GTACATGCCCGCCAGCTGGGTGATGAGGATCATGACCTGGCTGGACAT | |
| | GGTGGACACCAGCCTGAGCGGCTTCAAGCTGAAGGACTGCGTGATGTA | |
| | CGCCAGCGCCGTGGTGCTGCTGATCCTGATGACCGCCAGGACCGTGT | |
| | ACGACGACGGCGCCAGGAGGGTGTGGACCCTGATGAACGTGCTGACC | |
| | CTGGTGTACAAGGTGTACTACGGCAACGCCCTGGACCAGGCCATCAGC | |
| | ATGTGGGCCCTGATCATCAGCGTGACCAGCAACTACAGCGGCGTGGTG | |
| | ACCACCGTGATGTTCCTGGCCAGGGGCATCGTGTTCATGTGCGTGGAG | |
| | TACTGCCCCATCTTCTTCATCACCGGCAACACCCTGCAGTGCATCATGC | |
| | TGGTGTACTGCTTCCTGGGCTACTTCTGCACCTGCTACTTCGGCCTGTT | |
| | CTGCCTGCTGAACAGGTACTTCAGGCTGACCCTGGGCGTGTACGACTA | |
| | CCTGGTGAGCACCCAGGAGTTCAGGTACATGAACAGCCAGGGCCTGCT | |
| | GCCCCCCAAGAACAGCATCGACGCCTTCAAGCTGAACATCAAGCTGCT | |
| | GGGCGTGGGCGGCAAGCCCTGCATCAAGGTGGCCACCGTGCAGGGAA | |
| | GCGGAGAGGGCAGGGGAAGTCTTCTAACATGCGGGGACGTGGAGGAA | |
| | AATCCCGGCCCCAGCAAGATGAGCGACGTGAAGTGCACCAGCGTGGT | |
| | GCTGCTGAGCGTGCTGCAGCAGCTGAGGGTGGAGAGCAGCAGCAAGC | |
| | TGTGGGCCCAGTGCGTGCAGCTGCACAACGACATCCTGCTGGCCAAG | |
| | GACACCACCGAGGCCTTCGAGAAGATGGTGAGCCTGCTGAGCGTGCT | |
| | GCTGAGCATGCAGGGCGCCGTGGACATCAACAAGCTGTGCGAGGAGA | |
| | TGCTGGACAACAGGGCCACCCTGCAGGGAAGCGGACAGTGTACTAATT | |
| | ATGCTCTCTTGAAATTGGCTGGAGATGTTGAGAGCAACCCAGGTCCCG | |
| | CCATCGCCAGCGAGTTCAGCAGCCTGCCCAGCTACGCCGCCTTCGCCA | |
| | CCGCCCAGGAGGCCTACGAGCAGGCCGTGGCCAACGGCGACAGCGA | |
| | GGTGGTGCTGAAGAAGCTGAAGAAGAGCCTGAACGTGGCCAAGAGCG | |
| | AGTTCGACAGGGACGCCGCCATGCAGAGGAAGCTGGAGAAGATGGCC | |
| | GACCAGGCCATGACCCAGATGTACAAGCAGGCCAGGAGCGAGGACAA | |
| | GAGGGCCAAGGTGACCAGCGCCATGCAGACCATGCTGTTCACCATGCT | |
| | GAGGAAGCTGGACAACGACGCCCTGAACAACATCATCAACAACGCCAG | |
| | GGACGGCTGCGTGCCCCTGAACATCATCCCCCTGACCACCGCCGCCA | |
| | AGCTGATGGTGGTGATCCCCGACTACAACACCTACAAGAACACCTGCG | |
| | ACGGCACCACCTTCACCTACGCCAGCGCCCTGTGGGAGATCCAGCAG | |
| | GTGGTGGACGCCGACAGCAAGATCGTGCAGCTGAGCGAGATCAGCAT | |
| | GGACAACAGCCCCAACCTGGCCTGGCCCCTGATCGTGACCGCCCTGA | |
| | GGGCCAACAGCGCCGTGAAGCTGCAGTGATGA | |
| 25 | ATGTTCGTGTTCCTGGTGCTGCTGCCCCTGGTGAGCAGCCAGTGCGTG | 310 |
| Pan-CoV | AACCTGACCACCAGGACCCAGCTGCCCCCCGCCTACACCAACAGCTTC | |
| mRNA | ACCAGGGGCGTGTACTACCCCGACAAGGTGTTCAGGAGCAGCGTGCT | |
| HexaPro- | GCACAGCACCCAGGACCTGTTCCTGCCCTTCTTCAGCAACGTGACCTG | |
| NSP6-NSP7- | GTTCCACGCCATCCACGTGAGCGGCACCAACGGCACCAAGAGGTTCGA | |
| NSP8 | CAACCCCGTGCTGCCCTTCAACGACGGCGTGTACTTCGCCAGCACCGA | |
| | GAAGAGCAACATCATCAGGGGCTGGATCTTCGGCACCACCCTGGACAG | |
| | CAAGACCCAGAGCCTGCTGATCGTGAACAACGCCACCAACGTGGTGAT | |
| | CAAGGTGTGCGAGTTCCAGTTCTGCAACGACCCCTTCCTGGGCGTGTA | |
| | CTACCACAAGAACAACAAGAGCTGGATGGAGAGCGAGTTCAGGGTGTA | |
| | CAGCAGCGCCAACAACTGCACCTTCGAGTACGTGAGCCAGCCCTTCCT | |
| | GATGGACCTGGAGGGCAAGCAGGGCAACTTCAAGAACCTGAGGGAGT | |
| | TCGTGTTCAAGAACATCGACGGCTACTTCAAGATCTACAGCAAGCACAC | |
| | CCCCATCAACCTGGTGAGGGACCTGCCCCAGGGCTTCAGCGCCCTGG | |
| | AGCCCCTGGTGGACCTGCCCATCGGCATCAACATCACCAGGTTCCAGA | |
| | CCCTGCTGGCCCTGCACAGGAGCTACCTGACCCCCGGCGACAGCAGC | |
| | AGCGGCTGGACCGCCGGCGCCGCCGCCTACTACGTGGGCTACCTGCA | |
| | GCCCAGGACCTTCCTGCTGAAGTACAACGAGAACGGCACCATCACCGA | |
| | CGCCGTGGACTGCGCCCTGGACCCCCTGAGCGAGACCAAGTGCACCC | |
| | TGAAGAGCTTCACCGTGGAGAAGGGCATCTACCAGACCAGCAACTTCA | |
| | GGGTGCAGCCCACCGAGAGCATCGTGAGGTTCCCCAACATCACCAACC | |
| | TGTGCCCCTTCGGCGAGGTGTTCAACGCCACCAGGTTCGCCAGCGTGT | |
| | ACGCCTGGAACAGGAAGAGGATCAGCAACTGCGTGGCCGACTACAGC | |
| | GTGCTGTACAACAGCGCCAGCTTCAGCACCTTCAAGTGCTACGGCGTG | |
| | AGCCCCACCAAGCTGAACGACCTGTGCTTCACCAACGTGTACGCCGAC | |
| | AGCTTCGTGATCAGGGGCGACGAGGTGAGGCAGATCGCCCCCGGCCA | |
| | GACCGGCAAGATCGCCGACTACAACTACAAGCTGCCCGACGACTTCAC | |
| | CGGCTGCGTGATCGCCTGGAACAGCAACAACCTGGACAGCAAGGTGG | |
| | GCGGCAACTACAACTACCTGTACAGGCTGTTCAGGAAGAGCAACCTGA | |
| | AGCCCTTCGAGAGGGACATCAGCACCGAGATCTACCAGGCCGGCAGC | |
| | ACCCCCTGCAACGGCGTGGAGGGCTTCAACTGCTACTTCCCCCTGCAG | |
| | AGCTACGGCTTCCAGCCCACCAACGGCGTGGGCTACCAGCCCTACAG | |
| | GGTGGTGGTGCTGAGCTTCGAGCTGCTGCACGCCCCCGCCACCGTGT | |
| | GCGGCCCCAAGAAGAGCACCAACCTGGTGAAGAACAAGTGCGTGAACT | |
| | TCAACTTCAACGGCCTGACCGGCACCGGCGTGCTGACCGAGAGCAACA | |
| | AGAAGTTCCTGCCCTTCCAGCAGTTCGGCAGGGACATCGCCGACACCA | |
| | CCGACGCCGTGAGGGACCCCCAGACCCTGGAGATCCTGGACATCACC | |
| | CCCTGCAGCTTCGGCGGCGTGAGCGTGATCACCCCCGGCACCAACAC | |
| | CAGCAACCAGGTGGCCGTGCTGTACCAGGACGTGAACTGCACCGAGG | |
| | TGCCCGTGGCCATCCACGCCGACCAGCTGACCCCCACCTGGAGGGTG | |
| | TACAGCACCGGCAGCAACGTGTTCCAGACCAGGGCCGGCTGCCTGAT | |
| | CGGCGCCGAGCACGTGAACAACAGCTACGAGTGCGACATCCCCATCG | |
| | GCGCCGGCATCTGCGCCAGCTACCAGACCCAGACCAACAGCCCCGGC | |
| | AGCGCCAGCAGCGTGGCCAGCCAGAGCATCATCGCCTACACCATGAG | |
| | CCTGGGCGCCGAGAACAGCGTGGCCTACAGCAACAACAGCATCGCCA | |
| | TCCCCACCAACTTCACCATCAGCGTGACCACCGAGATCCTGCCCGTGA | |
| | GCATGACCAAGACCAGCGTGGACTGCACCATGTACATCTGCGGCGACA | |
| | GCACCGAGTGCAGCAACCTGCTGCTGCAGTACGGCAGCTTCTGCACCC | |
| | AGCTGAACAGGGCCCTGACCGGCATCGCCGTGGAGCAGGACAAGAAC | |
| | ACCCAGGAGGTGTTCGCCCAGGTGAAGCAGATCTACAAGACCCCCCCC | |
| | ATCAAGGACTTCGGCGGCTTCAACTTCAGCCAGATCCTGCCCGACCCC | |
| | AGCAAGCCCAGCAAGAGGAGCCCCATCGAGGACCTGCTGTTCAACAAG | |
| | GTGACCCTGGCCGACGCCGGCTTCATCAAGCAGTACGGCGACTGCCT | |
| | GGGCGACATCGCCGCCAGGGACCTGATCTGCGCCCAGAAGTTCAACG | |
| | GCCTGACCGTGCTGCCCCCCCTGCTGACCGACGAGATGATCGCCCAG | |
| | TACACCAGCGCCCTGCTGGCCGGCACCATCACCAGCGGCTGGACCTT | |
| | CGGCGCCGGCCCCGCCCTGCAGATCCCCTTCCCCATGCAGATGGCCT | |
| | ACAGGTTCAACGGCATCGGCGTGACCCAGAACGTGCTGTACGAGAACC | |
| | AGAAGCTGATCGCCAACCAGTTCAACAGCGCCATCGGCAAGATCCAGG | |
| | ACAGCCTGAGCAGCACCCCCAGCGCCCTGGGCAAGCTGCAGGACGTG | |
| | GTGAACCAGAACGCCCAGGCCCTGAACACCCTGGTGAAGCAGCTGAG | |
| | CAGCAACTTCGGCGCCATCAGCAGCGTGCTGAACGACATCCTGAGCAG | |
| | GCTGGACCCCCCCGAGGCCGAGGTGCAGATCGACAGGCTGATCACCG | |
| | GCAGGCTGCAGAGCCTGCAGACCTACGTGACCCAGCAGCTGATCAGG | |
| | GCCGCCGAGATCAGGGCCAGCGCCAACCTGGCCGCCACCAAGATGAG | |
| | CGAGTGCGTGCTGGGCCAGAGCAAGAGGGTGGACTTCTGCGGCAAGG | |
| | GCTACCACCTGATGAGCTTCCCCCAGAGCGCCCCCCACGGCGTGGTG | |
| | TTCCTGCACGTGACCTACGTGCCCGCCCAGGAGAAGAACTTCACCACC | |
| | GCCCCCGCCATCTGCCACGACGGCAAGGCCCACTTCCCCAGGGAGGG | |
| | CGTGTTCGTGAGCAACGGCACCCACTGGTTCGTGACCCAGAGGAACTT | |
| | CTACGAGCCCCAGATCATCACCACCGACAACACCTTCGTGAGCGGCAA | |
| | CTGCGACGTGGTGATCGGCATCGTGAACAACACCGTGTACGACCCCCT | |
| | GCAGCCCGAGCTGGACAGCTTCAAGGAGGAGCTGGACAAGTACTTCAA | |
| | GAACCACACCAGCCCCGACGTGGACCTGGGCGACATCAGCGGCATCA | |
| | ACGCCAGCGTGGTGAACATCCAGAAGGAGATCGACAGGCTGAACGAG | |
| | GTGGCCAAGAACCTGAACGAGAGCCTGATCGACCTGCAGGAGCTGGG | |
| | CAAGTACGAGCAGTACATCAAGTGGCCCTGGTACATCTGGCTGGGCTT | |
| | CATCGCCGGCCTGATCGCCATCGTGATGGTGACCATCATGCTGTGCTG | |
| | CATGACCAGCTGCTGCAGCTGCCTGAAGGGCTGCTGCAGCTGCGGCA | |
| | GCTGCTGCAAGTTCGACGAGGACGACAGCGAGCCCGTGCTGAAGGGC | |
| | GTGAAGCTGCACTACACCGGAAGCGGAGCCACGAACTTCTCTCTGTTA | |
| | AAGCAAGCAGGAGATGTTGAAGAAAACCCCGGGCCTAGCGCCGTGAA | |
| | GAGGACCATCAAGGGCACCCACCACTGGCTGCTGCTGACCATCCTGAC | |
| | CAGCCTGCTGGTGCTGGTGCAGAGCACCCAGTGGAGCCTGTTCTTCTT | |
| | CCTGTACGAGAACGCCTTCCTGCCCTTCGCCATGGGCATCATCGCCAT | |
| | GAGCGCCTTCGCCATGATGTTCGTGAAGCACAAGCACGCCTTCCTGTG | |
| | CCTGTTCCTGCTGCCCAGCCTGGCCACCGTGGCCTACTTCAACATGGT | |
| | GTACATGCCCGCCAGCTGGGTGATGAGGATCATGACCTGGCTGGACAT | |
| | GGTGGACACCAGCCTGAGCGGCTTCAAGCTGAAGGACTGCGTGATGTA | |
| | CGCCAGCGCCGTGGTGCTGCTGATCCTGATGACCGCCAGGACCGTGT | |
| | ACGACGACGGCGCCAGGAGGGTGTGGACCCTGATGAACGTGCTGACC | |
| | CTGGTGTACAAGGTGTACTACGGCAACGCCCTGGACCAGGCCATCAGC | |
| | ATGTGGGCCCTGATCATCAGCGTGACCAGCAACTACAGCGGCGTGGTG | |
| | ACCACCGTGATGTTCCTGGCCAGGGGCATCGTGTTCATGTGCGTGGAG | |
| | TACTGCCCCATCTTCTTCATCACCGGCAACACCCTGCAGTGCATCATGC | |
| | TGGTGTACTGCTTCCTGGGCTACTTCTGCACCTGCTACTTCGGCCTGTT | |
| | CTGCCTGCTGAACAGGTACTTCAGGCTGACCCTGGGCGTGTACGACTA | |
| | CCTGGTGAGCACCCAGGAGTTCAGGTACATGAACAGCCAGGGCCTGCT | |
| | GCCCCCCAAGAACAGCATCGACGCCTTCAAGCTGAACATCAAGCTGCT | |
| | GGGCGTGGGCGGCAAGCCCTGCATCAAGGTGGCCACCGTGCAGGGAA | |
| | GCGGAGAGGGCAGGGGAAGTCTTCTAACATGCGGGGACGTGGAGGAA | |
| | AATCCCGGCCCCAGCAAGATGAGCGACGTGAAGTGCACCAGCGTGGT | |
| | GCTGCTGAGCGTGCTGCAGCAGCTGAGGGTGGAGAGCAGCAGCAAGC | |
| | TGTGGGCCCAGTGCGTGCAGCTGCACAACGACATCCTGCTGGCCAAG | |
| | GACACCACCGAGGCCTTCGAGAAGATGGTGAGCCTGCTGAGCGTGCT | |
| | GCTGAGCATGCAGGGCGCCGTGGACATCAACAAGCTGTGCGAGGAGA | |
| | TGCTGGACAACAGGGCCACCCTGCAGGGAAGCGGACAGTGTACTAATT | |
| | ATGCTCTCTTGAAATTGGCTGGAGATGTTGAGAGCAACCCAGGTCCCG | |
| | CCATCGCCAGCGAGTTCAGCAGCCTGCCCAGCTACGCCGCCTTCGCCA | |
| | CCGCCCAGGAGGCCTACGAGCAGGCCGTGGCCAACGGCGACAGCGA | |
| | GGTGGTGCTGAAGAAGCTGAAGAAGAGCCTGAACGTGGCCAAGAGCG | |
| | AGTTCGACAGGGACGCCGCCATGCAGAGGAAGCTGGAGAAGATGGCC | |
| | GACCAGGCCATGACCCAGATGTACAAGCAGGCCAGGAGCGAGGACAA | |
| | GAGGGCCAAGGTGACCAGCGCCATGCAGACCATGCTGTTCACCATGCT | |
| | GAGGAAGCTGGACAACGACGCCCTGAACAACATCATCAACAACGCCAG | |
| | GGACGGCTGCGTGCCCCTGAACATCATCCCCCTGACCACCGCCGCCA | |
| | AGCTGATGGTGGTGATCCCCGACTACAACACCTACAAGAACACCTGCG | |
| | ACGGCACCACCTTCACCTACGCCAGCGCCCTGTGGGAGATCCAGCAG | |
| | GTGGTGGACGCCGACAGCAAGATCGTGCAGCTGAGCGAGATCAGCAT | |
| | GGACAACAGCCCCAACCTGGCCTGGCCCCTGATCGTGACCGCCCTGA | |
| | GGGCCAACAGCGCCGTGAAGCTGCAGTGATGA | |
| 28 | ATGTTCGTGTTCCTGGTGCTGCTGCCCCTGGTGAGCAGCCAGTGCGTG | 311 |
| Pan-CoV | AACCTGACCACCAGGACCCAGCTGCCCCCCGCCTACACCAACAGCTTC | |
| mRNA Spike- | ACCAGGGGCGTGTACTACCCCGACAAGGTGTTCAGGAGCAGCGTGCT | |
| NSP9-NSP10- | GCACAGCACCCAGGACCTGTTCCTGCCCTTCTTCAGCAACGTGACCTG | |
| NSP13 | GTTCCACGCCATCCACGTGAGCGGCACCAACGGCACCAAGAGGTTCGA | |
| | CAACCCCGTGCTGCCCTTCAACGACGGCGTGTACTTCGCCAGCACCGA | |
| | GAAGAGCAACATCATCAGGGGCTGGATCTTCGGCACCACCCTGGACAG | |
| | CAAGACCCAGAGCCTGCTGATCGTGAACAACGCCACCAACGTGGTGAT | |
| | CAAGGTGTGCGAGTTCCAGTTCTGCAACGACCCCTTCCTGGGCGTGTA | |
| | CTACCACAAGAACAACAAGAGCTGGATGGAGAGCGAGTTCAGGGTGTA | |
| | CAGCAGCGCCAACAACTGCACCTTCGAGTACGTGAGCCAGCCCTTCCT | |
| | GATGGACCTGGAGGGCAAGCAGGGCAACTTCAAGAACCTGAGGGAGT | |
| | TCGTGTTCAAGAACATCGACGGCTACTTCAAGATCTACAGCAAGCACAC | |
| | CCCCATCAACCTGGTGAGGGACCTGCCCCAGGGCTTCAGCGCCCTGG | |
| | AGCCCCTGGTGGACCTGCCCATCGGCATCAACATCACCAGGTTCCAGA | |
| | CCCTGCTGGCCCTGCACAGGAGCTACCTGACCCCCGGCGACAGCAGC | |
| | AGCGGCTGGACCGCCGGCGCCGCCGCCTACTACGTGGGCTACCTGCA | |
| | GCCCAGGACCTTCCTGCTGAAGTACAACGAGAACGGCACCATCACCGA | |
| | CGCCGTGGACTGCGCCCTGGACCCCCTGAGCGAGACCAAGTGCACCC | |
| | TGAAGAGCTTCACCGTGGAGAAGGGCATCTACCAGACCAGCAACTTCA | |
| | GGGTGCAGCCCACCGAGAGCATCGTGAGGTTCCCCAACATCACCAACC | |
| | TGTGCCCCTTCGGCGAGGTGTTCAACGCCACCAGGTTCGCCAGCGTGT | |
| | ACGCCTGGAACAGGAAGAGGATCAGCAACTGCGTGGCCGACTACAGC | |
| | GTGCTGTACAACAGCGCCAGCTTCAGCACCTTCAAGTGCTACGGCGTG | |
| | AGCCCCACCAAGCTGAACGACCTGTGCTTCACCAACGTGTACGCCGAC | |
| | AGCTTCGTGATCAGGGGCGACGAGGTGAGGCAGATCGCCCCCGGCCA | |
| | GACCGGCAAGATCGCCGACTACAACTACAAGCTGCCCGACGACTTCAC | |
| | CGGCTGCGTGATCGCCTGGAACAGCAACAACCTGGACAGCAAGGTGG | |
| | GCGGCAACTACAACTACCTGTACAGGCTGTTCAGGAAGAGCAACCTGA | |
| | AGCCCTTCGAGAGGGACATCAGCACCGAGATCTACCAGGCCGGCAGC | |
| | ACCCCCTGCAACGGCGTGGAGGGCTTCAACTGCTACTTCCCCCTGCAG | |
| | AGCTACGGCTTCCAGCCCACCAACGGCGTGGGCTACCAGCCCTACAG | |
| | GGTGGTGGTGCTGAGCTTCGAGCTGCTGCACGCCCCCGCCACCGTGT | |
| | GCGGCCCCAAGAAGAGCACCAACCTGGTGAAGAACAAGTGCGTGAACT | |
| | TCAACTTCAACGGCCTGACCGGCACCGGCGTGCTGACCGAGAGCAACA | |
| | AGAAGTTCCTGCCCTTCCAGCAGTTCGGCAGGGACATCGCCGACACCA | |
| | CCGACGCCGTGAGGGACCCCCAGACCCTGGAGATCCTGGACATCACC | |
| | CCCTGCAGCTTCGGCGGCGTGAGCGTGATCACCCCCGGCACCAACAC | |
| | CAGCAACCAGGTGGCCGTGCTGTACCAGGACGTGAACTGCACCGAGG | |
| | TGCCCGTGGCCATCCACGCCGACCAGCTGACCCCCACCTGGAGGGTG | |
| | TACAGCACCGGCAGCAACGTGTTCCAGACCAGGGCCGGCTGCCTGAT | |
| | CGGCGCCGAGCACGTGAACAACAGCTACGAGTGCGACATCCCCATCG | |
| | GCGCCGGCATCTGCGCCAGCTACCAGACCCAGACCAACAGCCCCAGG | |
| | AGGGCCAGGAGCGTGGCCAGCCAGAGCATCATCGCCTACACCATGAG | |
| | CCTGGGCGCCGAGAACAGCGTGGCCTACAGCAACAACAGCATCGCCA | |
| | TCCCCACCAACTTCACCATCAGCGTGACCACCGAGATCCTGCCCGTGA | |
| | GCATGACCAAGACCAGCGTGGACTGCACCATGTACATCTGCGGCGACA | |
| | GCACCGAGTGCAGCAACCTGCTGCTGCAGTACGGCAGCTTCTGCACCC | |
| | AGCTGAACAGGGCCCTGACCGGCATCGCCGTGGAGCAGGACAAGAAC | |
| | ACCCAGGAGGTGTTCGCCCAGGTGAAGCAGATCTACAAGACCCCCCCC | |
| | ATCAAGGACTTCGGCGGCTTCAACTTCAGCCAGATCCTGCCCGACCCC | |
| | AGCAAGCCCAGCAAGAGGAGCTTCATCGAGGACCTGCTGTTCAACAAG | |
| | GTGACCCTGGCCGACGCCGGCTTCATCAAGCAGTACGGCGACTGCCT | |
| | GGGCGACATCGCCGCCAGGGACCTGATCTGCGCCCAGAAGTTCAACG | |
| | GCCTGACCGTGCTGCCCCCCCTGCTGACCGACGAGATGATCGCCCAG | |
| | TACACCAGCGCCCTGCTGGCCGGCACCATCACCAGCGGCTGGACCTT | |
| | CGGCGCCGGCGCCGCCCTGCAGATCCCCTTCGCCATGCAGATGGCCT | |
| | ACAGGTTCAACGGCATCGGCGTGACCCAGAACGTGCTGTACGAGAACC | |
| | AGAAGCTGATCGCCAACCAGTTCAACAGCGCCATCGGCAAGATCCAGG | |
| | ACAGCCTGAGCAGCACCGCCAGCGCCCTGGGCAAGCTGCAGGACGTG | |
| | GTGAACCAGAACGCCCAGGCCCTGAACACCCTGGTGAAGCAGCTGAG | |
| | CAGCAACTTCGGCGCCATCAGCAGCGTGCTGAACGACATCCTGAGCAG | |
| | GCTGGACCCCCCCGAGGCCGAGGTGCAGATCGACAGGCTGATCACCG | |
| | GCAGGCTGCAGAGCCTGCAGACCTACGTGACCCAGCAGCTGATCAGG | |
| | GCCGCCGAGATCAGGGCCAGCGCCAACCTGGCCGCCACCAAGATGAG | |
| | CGAGTGCGTGCTGGGCCAGAGCAAGAGGGTGGACTTCTGCGGCAAGG | |
| | GCTACCACCTGATGAGCTTCCCCCAGAGCGCCCCCCACGGCGTGGTG | |
| | TTCCTGCACGTGACCTACGTGCCCGCCCAGGAGAAGAACTTCACCACC | |
| | GCCCCCGCCATCTGCCACGACGGCAAGGCCCACTTCCCCAGGGAGGG | |
| | CGTGTTCGTGAGCAACGGCACCCACTGGTTCGTGACCCAGAGGAACTT | |
| | CTACGAGCCCCAGATCATCACCACCGACAACACCTTCGTGAGCGGCAA | |
| | CTGCGACGTGGTGATCGGCATCGTGAACAACACCGTGTACGACCCCCT | |
| | GCAGCCCGAGCTGGACAGCTTCAAGGAGGAGCTGGACAAGTACTTCAA | |
| | GAACCACACCAGCCCCGACGTGGACCTGGGCGACATCAGCGGCATCA | |
| | ACGCCAGCGTGGTGAACATCCAGAAGGAGATCGACAGGCTGAACGAG | |
| | GTGGCCAAGAACCTGAACGAGAGCCTGATCGACCTGCAGGAGCTGGG | |
| | CAAGTACGAGCAGTACATCAAGTGGCCCTGGTACATCTGGCTGGGCTT | |
| | CATCGCCGGCCTGATCGCCATCGTGATGGTGACCATCATGCTGTGCTG | |
| | CATGACCAGCTGCTGCAGCTGCCTGAAGGGCTGCTGCAGCTGCGGCA | |
| | GCTGCTGCAAGTTCGACGAGGACGACAGCGAGCCCGTGCTGAAGGGC | |
| | GTGAAGCTGCACTACACCGGAAGCGGAGCCACGAACTTCTCTCTGTTA | |
| | AAGCAAGCAGGAGATGTTGAAGAAAACCCCGGGCCTAACAACGAGCTG | |
| | AGCCCCGTGGCCCTGAGGCAGATGAGCTGCGCCGCCGGCACCACCCA | |
| | GACCGCCTGCACCGACGACAACGCCCTGGCCTACTACAACACCACCAA | |
| | GGGCGGCAGGTTCGTGCTGGCCCTGCTGAGCGACCTGCAGGACCTGA | |
| | AGTGGGCCAGGTTCCCCAAGAGCGACGGCACCGGCACCATCTACACC | |
| | GAGCTGGAGCCCCCCTGCAGGTTCGTGACCGACACCCCCAAGGGCCC | |
| | CAAGGTGAAGTACCTGTACTTCATCAAGGGCCTGAACAACCTGAACAG | |
| | GGGCATGGTGCTGGGCAGCCTGGCCGCCACCGTGAGGCTGCAGGGAA | |
| | GCGGAGAGGGCAGGGGAAGTCTTCTAACATGCGGGGACGTGGAGGAA | |
| | AATCCCGGCCCCGCCGGCAACGCCACCGAGGTGCCCGCCAACAGCAC | |
| | CGTGCTGAGCTTCTGCGCCTTCGCCGTGGACGCCGCCAAGGCCTACAA | |
| | GGACTACCTGGCCAGCGGCGGCCAGCCCATCACCAACTGCGTGAAGA | |
| | TGCTGTGCACCCACACCGGCACCGGCCAGGCCATCACCGTGACCCCC | |
| | GAGGCCAACATGGACCAGGAGAGCTTCGGCGGCGCCAGCTGCTGCCT | |
| | GTACTGCAGGTGCCACATCGACCACCCCAACCCCAAGGGCTTCTGCGA | |
| | CCTGAAGGGCAAGTACGTGCAGATCCCCACCACCTGCGCCAACGACCC | |
| | CGTGGGCTTCACCCTGAAGAACACCGTGTGCACCGTGTGCGGCATGTG | |
| | GAAGGGCTACGGCTGCAGCTGCGACCAGCTGAGGGAGCCCATGCTGC | |
| | AGGGAAGCGGACAGTGTACTAATTATGCTCTCTTGAAATTGGCTGGAGA | |
| | TGTTGAGAGCAACCCAGGTCCCGCCGTGGGCGCCTGCGTGCTGTGCA | |
| | ACAGCCAGACCAGCCTGAGGTGCGGCGCCTGCATCAGGAGGCCCTTC | |
| | CTGTGCTGCAAGTGCTGCTACGACCACGTGATCAGCACCAGCCACAAG | |
| | CTGGTGCTGAGCGTGAACCCCTACGTGTGCAACGCCCCCGGCTGCGA | |
| | CGTGACCGACGTGACCCAGCTGTACCTGGGCGGCATGAGCTACTACTG | |
| | CAAGAGCCACAAGCCCCCCATCAGCTTCCCCCTGTGCGCCAACGGCCA | |
| | GGTGTTCGGCCTGTACAAGAACACCTGCGTGGGCAGCGACAACGTGAC | |
| | CGACTTCAACGCCATCGCCACCTGCGACTGGACCAACGCCGGCGACTA | |
| | CATCCTGGCCAACACCTGCACCGAGAGGCTGAAGCTGTTCGCCGCCGA | |
| | GACCCTGAAGGCCACCGAGGAGACCTTCAAGCTGAGCTACGGCATCG | |
| | CCACCGTGAGGGAGGTGCTGAGCGACAGGGAGCTGCACCTGAGCTGG | |
| | GAGGTGGGCAAGCCCAGGCCCCCCCTGAACAGGAACTACGTGTTCAC | |
| | CGGCTACAGGGTGACCAAGAACAGCAAGGTGCAGATCGGCGAGTACA | |
| | CCTTCGAGAAGGGCGACTACGGCGACGCCGTGGTGTACAGGGGCACC | |
| | ACCACCTACAAGCTGAACGTGGGCGACTACTTCGTGCTGACCAGCCAC | |
| | ACCGTGATGCCCCTGAGCGCCCCCACCCTGGTGCCCCAGGAGCACTA | |
| | CGTGAGGATCACCGGCCTGTACCCCACCCTGAACATCAGCGACGAGTT | |
| | CAGCAGCAACGTGGCCAACTACCAGAAGGTGGGCATGCAGAAGTACAG | |
| | CACCCTGCAGGGCCCCCCCGGCACCGGCAAGAGCCACTTCGCCATCG | |
| | GCCTGGCCCTGTACTACCCCAGCGCCAGGATCGTGTACACCGCCTGCA | |
| | GCCACGCCGCCGTGGACGCCCTGTGCGAGAAGGCCCTGAAGTACCTG | |
| | CCCATCGACAAGTGCAGCAGGATCATCCCCGCCAGGGCCAGGGTGGA | |
| | GTGCTTCGACAAGTTCAAGGTGAACAGCACCCTGGAGCAGTACGTGTT | |
| | CTGCACCGTGAACGCCCTGCCCGAGACCACCGCCGACATCGTGGTGTT | |
| | CGACGAGATCAGCATGGCCACCAACTACGACCTGAGCGTGGTGAACGC | |
| | CAGGCTGAGGGCCAAGCACTACGTGTACATCGGCGACCCCGCCCAGC | |
| | TGCCCGCCCCCAGGACCCTGCTGACCAAGGGCACCCTGGAGCCCGAG | |
| | TACTTCAACAGCGTGTGCAGGCTGATGAAGACCATCGGCCCCGACATG | |
| | TTCCTGGGCACCTGCAGGAGGTGCCCCGCCGAGATCGTGGACACCGT | |
| | GAGCGCCCTGGTGTACGACAACAAGCTGAAGGCCCACAAGGACAAGA | |
| | GCGCCCAGTGCTTCAAGATGTTCTACAAGGGCGTGATCACCCACGACG | |
| | TGAGCAGCGCCATCAACAGGCCCCAGATCGGCGTGGTGAGGGAGTTC | |
| | CTGACCAGGAACCCCGCCTGGAGGAAGGCCGTGTTCATCAGCCCCTAC | |
| | AACAGCCAGAACGCCGTGGCCAGCAAGATCCTGGGCCTGCCCACCCA | |
| | GACCGTGGACAGCAGCCAGGGCAGCGAGTACGACTACGTGATCTTCAC | |
| | CCAGACCACCGAGACCGCCCACAGCTGCAACGTGAACAGGTTCAACGT | |
| | GGCCATCACCAGGGCCAAGGTGGGCATCCTGTGCATCATGAGCGACA | |
| | GGGACCTGTACGACAAGCTGCAGTTCACCAGCCTGGAGATCCCCAGGA | |
| | GGAACGTGGCCACCCTGCAGTGATGA | |
| 27 | ATGTTCGTGTTCCTGGTGCTGCTGCCCCTGGTGAGCAGCCAGTGCGTG | 312 |
| Pan-CoV | AACCTGACCACCAGGACCCAGCTGCCCCCCGCCTACACCAACAGCTTC | |
| mRNA | ACCAGGGGCGTGTACTACCCCGACAAGGTGTTCAGGAGCAGCGTGCT | |
| HexaPro- | GCACAGCACCCAGGACCTGTTCCTGCCCTTCTTCAGCAACGTGACCTG | |
| NSP9-NSP10- | GTTCCACGCCATCCACGTGAGCGGCACCAACGGCACCAAGAGGTTCGA | |
| NSP13 | CAACCCCGTGCTGCCCTTCAACGACGGCGTGTACTTCGCCAGCACCGA | |
| | GAAGAGCAACATCATCAGGGGCTGGATCTTCGGCACCACCCTGGACAG | |
| | CAAGACCCAGAGCCTGCTGATCGTGAACAACGCCACCAACGTGGTGAT | |
| | CAAGGTGTGCGAGTTCCAGTTCTGCAACGACCCCTTCCTGGGCGTGTA | |
| | CTACCACAAGAACAACAAGAGCTGGATGGAGAGCGAGTTCAGGGTGTA | |
| | CAGCAGCGCCAACAACTGCACCTTCGAGTACGTGAGCCAGCCCTTCCT | |
| | GATGGACCTGGAGGGCAAGCAGGGCAACTTCAAGAACCTGAGGGAGT | |
| | TCGTGTTCAAGAACATCGACGGCTACTTCAAGATCTACAGCAAGCACAC | |
| | CCCCATCAACCTGGTGAGGGACCTGCCCCAGGGCTTCAGCGCCCTGG | |
| | AGCCCCTGGTGGACCTGCCCATCGGCATCAACATCACCAGGTTCCAGA | |
| | CCCTGCTGGCCCTGCACAGGAGCTACCTGACCCCCGGCGACAGCAGC | |
| | AGCGGCTGGACCGCCGGCGCCGCCGCCTACTACGTGGGCTACCTGCA | |
| | GCCCAGGACCTTCCTGCTGAAGTACAACGAGAACGGCACCATCACCGA | |
| | CGCCGTGGACTGCGCCCTGGACCCCCTGAGCGAGACCAAGTGCACCC | |
| | TGAAGAGCTTCACCGTGGAGAAGGGCATCTACCAGACCAGCAACTTCA | |
| | GGGTGCAGCCCACCGAGAGCATCGTGAGGTTCCCCAACATCACCAACC | |
| | TGTGCCCCTTCGGCGAGGTGTTCAACGCCACCAGGTTCGCCAGCGTGT | |
| | ACGCCTGGAACAGGAAGAGGATCAGCAACTGCGTGGCCGACTACAGC | |
| | GTGCTGTACAACAGCGCCAGCTTCAGCACCTTCAAGTGCTACGGCGTG | |
| | AGCCCCACCAAGCTGAACGACCTGTGCTTCACCAACGTGTACGCCGAC | |
| | AGCTTCGTGATCAGGGGCGACGAGGTGAGGCAGATCGCCCCCGGCCA | |
| | GACCGGCAAGATCGCCGACTACAACTACAAGCTGCCCGACGACTTCAC | |
| | CGGCTGCGTGATCGCCTGGAACAGCAACAACCTGGACAGCAAGGTGG | |
| | GCGGCAACTACAACTACCTGTACAGGCTGTTCAGGAAGAGCAACCTGA | |
| | AGCCCTTCGAGAGGGACATCAGCACCGAGATCTACCAGGCCGGCAGC | |
| | ACCCCCTGCAACGGCGTGGAGGGCTTCAACTGCTACTTCCCCCTGCAG | |
| | AGCTACGGCTTCCAGCCCACCAACGGCGTGGGCTACCAGCCCTACAG | |
| | GGTGGTGGTGCTGAGCTTCGAGCTGCTGCACGCCCCCGCCACCGTGT | |
| | GCGGCCCCAAGAAGAGCACCAACCTGGTGAAGAACAAGTGCGTGAACT | |
| | TCAACTTCAACGGCCTGACCGGCACCGGCGTGCTGACCGAGAGCAACA | |
| | AGAAGTTCCTGCCCTTCCAGCAGTTCGGCAGGGACATCGCCGACACCA | |
| | CCGACGCCGTGAGGGACCCCCAGACCCTGGAGATCCTGGACATCACC | |
| | CCCTGCAGCTTCGGCGGCGTGAGCGTGATCACCCCCGGCACCAACAC | |
| | CAGCAACCAGGTGGCCGTGCTGTACCAGGACGTGAACTGCACCGAGG | |
| | TGCCCGTGGCCATCCACGCCGACCAGCTGACCCCCACCTGGAGGGTG | |
| | TACAGCACCGGCAGCAACGTGTTCCAGACCAGGGCCGGCTGCCTGAT | |
| | CGGCGCCGAGCACGTGAACAACAGCTACGAGTGCGACATCCCCATCG | |
| | GCGCCGGCATCTGCGCCAGCTACCAGACCCAGACCAACAGCCCCGGC | |
| | AGCGCCAGCAGCGTGGCCAGCCAGAGCATCATCGCCTACACCATGAG | |
| | CCTGGGCGCCGAGAACAGCGTGGCCTACAGCAACAACAGCATCGCCA | |
| | TCCCCACCAACTTCACCATCAGCGTGACCACCGAGATCCTGCCCGTGA | |
| | GCATGACCAAGACCAGCGTGGACTGCACCATGTACATCTGCGGCGACA | |
| | GCACCGAGTGCAGCAACCTGCTGCTGCAGTACGGCAGCTTCTGCACCC | |
| | AGCTGAACAGGGCCCTGACCGGCATCGCCGTGGAGCAGGACAAGAAC | |
| | ACCCAGGAGGTGTTCGCCCAGGTGAAGCAGATCTACAAGACCCCCCCC | |
| | ATCAAGGACTTCGGCGGCTTCAACTTCAGCCAGATCCTGCCCGACCCC | |
| | AGCAAGCCCAGCAAGAGGAGCCCCATCGAGGACCTGCTGTTCAACAAG | |
| | GTGACCCTGGCCGACGCCGGCTTCATCAAGCAGTACGGCGACTGCCT | |
| | GGGCGACATCGCCGCCAGGGACCTGATCTGCGCCCAGAAGTTCAACG | |
| | GCCTGACCGTGCTGCCCCCCCTGCTGACCGACGAGATGATCGCCCAG | |
| | TACACCAGCGCCCTGCTGGCCGGCACCATCACCAGCGGCTGGACCTT | |
| | CGGCGCCGGCCCCGCCCTGCAGATCCCCTTCCCCATGCAGATGGCCT | |
| | ACAGGTTCAACGGCATCGGCGTGACCCAGAACGTGCTGTACGAGAACC | |
| | AGAAGCTGATCGCCAACCAGTTCAACAGCGCCATCGGCAAGATCCAGG | |
| | ACAGCCTGAGCAGCACCCCCAGCGCCCTGGGCAAGCTGCAGGACGTG | |
| | GTGAACCAGAACGCCCAGGCCCTGAACACCCTGGTGAAGCAGCTGAG | |
| | CAGCAACTTCGGCGCCATCAGCAGCGTGCTGAACGACATCCTGAGCAG | |
| | GCTGGACCCCCCCGAGGCCGAGGTGCAGATCGACAGGCTGATCACCG | |
| | GCAGGCTGCAGAGCCTGCAGACCTACGTGACCCAGCAGCTGATCAGG | |
| | GCCGCCGAGATCAGGGCCAGCGCCAACCTGGCCGCCACCAAGATGAG | |
| | CGAGTGCGTGCTGGGCCAGAGCAAGAGGGTGGACTTCTGCGGCAAGG | |
| | GCTACCACCTGATGAGCTTCCCCCAGAGCGCCCCCCACGGCGTGGTG | |
| | TTCCTGCACGTGACCTACGTGCCCGCCCAGGAGAAGAACTTCACCACC | |
| | GCCCCCGCCATCTGCCACGACGGCAAGGCCCACTTCCCCAGGGAGGG | |
| | CGTGTTCGTGAGCAACGGCACCCACTGGTTCGTGACCCAGAGGAACTT | |
| | CTACGAGCCCCAGATCATCACCACCGACAACACCTTCGTGAGCGGCAA | |
| | CTGCGACGTGGTGATCGGCATCGTGAACAACACCGTGTACGACCCCCT | |
| | GCAGCCCGAGCTGGACAGCTTCAAGGAGGAGCTGGACAAGTACTTCAA | |
| | GAACCACACCAGCCCCGACGTGGACCTGGGCGACATCAGCGGCATCA | |
| | ACGCCAGCGTGGTGAACATCCAGAAGGAGATCGACAGGCTGAACGAG | |
| | GTGGCCAAGAACCTGAACGAGAGCCTGATCGACCTGCAGGAGCTGGG | |
| | CAAGTACGAGCAGTACATCAAGTGGCCCTGGTACATCTGGCTGGGCTT | |
| | CATCGCCGGCCTGATCGCCATCGTGATGGTGACCATCATGCTGTGCTG | |
| | CATGACCAGCTGCTGCAGCTGCCTGAAGGGCTGCTGCAGCTGCGGCA | |
| | GCTGCTGCAAGTTCGACGAGGACGACAGCGAGCCCGTGCTGAAGGGC | |
| | GTGAAGCTGCACTACACCGGAAGCGGAGCCACGAACTTCTCTCTGTTA | |
| | AAGCAAGCAGGAGATGTTGAAGAAAACCCCGGGCCTAACAACGAGCTG | |
| | AGCCCCGTGGCCCTGAGGCAGATGAGCTGCGCCGCCGGCACCACCCA | |
| | GACCGCCTGCACCGACGACAACGCCCTGGCCTACTACAACACCACCAA | |
| | GGGCGGCAGGTTCGTGCTGGCCCTGCTGAGCGACCTGCAGGACCTGA | |
| | AGTGGGCCAGGTTCCCCAAGAGCGACGGCACCGGCACCATCTACACC | |
| | GAGCTGGAGCCCCCCTGCAGGTTCGTGACCGACACCCCCAAGGGCCC | |
| | CAAGGTGAAGTACCTGTACTTCATCAAGGGCCTGAACAACCTGAACAG | |
| | GGGCATGGTGCTGGGCAGCCTGGCCGCCACCGTGAGGCTGCAGGGAA | |
| | GCGGAGAGGGCAGGGGAAGTCTTCTAACATGCGGGGACGTGGAGGAA | |
| | AATCCCGGCCCCGCCGGCAACGCCACCGAGGTGCCCGCCAACAGCAC | |
| | CGTGCTGAGCTTCTGCGCCTTCGCCGTGGACGCCGCCAAGGCCTACAA | |
| | GGACTACCTGGCCAGCGGCGGCCAGCCCATCACCAACTGCGTGAAGA | |
| | TGCTGTGCACCCACACCGGCACCGGCCAGGCCATCACCGTGACCCCC | |
| | GAGGCCAACATGGACCAGGAGAGCTTCGGCGGCGCCAGCTGCTGCCT | |
| | GTACTGCAGGTGCCACATCGACCACCCCAACCCCAAGGGCTTCTGCGA | |
| | CCTGAAGGGCAAGTACGTGCAGATCCCCACCACCTGCGCCAACGACCC | |
| | CGTGGGCTTCACCCTGAAGAACACCGTGTGCACCGTGTGCGGCATGTG | |
| | GAAGGGCTACGGCTGCAGCTGCGACCAGCTGAGGGAGCCCATGCTGC | |
| | AGGGAAGCGGACAGTGTACTAATTATGCTCTCTTGAAATTGGCTGGAGA | |
| | TGTTGAGAGCAACCCAGGTCCCGCCGTGGGCGCCTGCGTGCTGTGCA | |
| | ACAGCCAGACCAGCCTGAGGTGCGGCGCCTGCATCAGGAGGCCCTTC | |
| | CTGTGCTGCAAGTGCTGCTACGACCACGTGATCAGCACCAGCCACAAG | |
| | CTGGTGCTGAGCGTGAACCCCTACGTGTGCAACGCCCCCGGCTGCGA | |
| | CGTGACCGACGTGACCCAGCTGTACCTGGGCGGCATGAGCTACTACTG | |
| | CAAGAGCCACAAGCCCCCCATCAGCTTCCCCCTGTGCGCCAACGGCCA | |
| | GGTGTTCGGCCTGTACAAGAACACCTGCGTGGGCAGCGACAACGTGAC | |
| | CGACTTCAACGCCATCGCCACCTGCGACTGGACCAACGCCGGCGACTA | |
| | CATCCTGGCCAACACCTGCACCGAGAGGCTGAAGCTGTTCGCCGCCGA | |
| | GACCCTGAAGGCCACCGAGGAGACCTTCAAGCTGAGCTACGGCATCG | |
| | CCACCGTGAGGGAGGTGCTGAGCGACAGGGAGCTGCACCTGAGCTGG | |
| | GAGGTGGGCAAGCCCAGGCCCCCCCTGAACAGGAACTACGTGTTCAC | |
| | CGGCTACAGGGTGACCAAGAACAGCAAGGTGCAGATCGGCGAGTACA | |
| | CCTTCGAGAAGGGCGACTACGGCGACGCCGTGGTGTACAGGGGCACC | |
| | ACCACCTACAAGCTGAACGTGGGCGACTACTTCGTGCTGACCAGCCAC | |
| | ACCGTGATGCCCCTGAGCGCCCCCACCCTGGTGCCCCAGGAGCACTA | |
| | CGTGAGGATCACCGGCCTGTACCCCACCCTGAACATCAGCGACGAGTT | |
| | CAGCAGCAACGTGGCCAACTACCAGAAGGTGGGCATGCAGAAGTACAG | |
| | CACCCTGCAGGGCCCCCCCGGCACCGGCAAGAGCCACTTCGCCATCG | |
| | GCCTGGCCCTGTACTACCCCAGCGCCAGGATCGTGTACACCGCCTGCA | |
| | GCCACGCCGCCGTGGACGCCCTGTGCGAGAAGGCCCTGAAGTACCTG | |
| | CCCATCGACAAGTGCAGCAGGATCATCCCCGCCAGGGCCAGGGTGGA | |
| | GTGCTTCGACAAGTTCAAGGTGAACAGCACCCTGGAGCAGTACGTGTT | |
| | CTGCACCGTGAACGCCCTGCCCGAGACCACCGCCGACATCGTGGTGTT | |
| | CGACGAGATCAGCATGGCCACCAACTACGACCTGAGCGTGGTGAACGC | |
| | CAGGCTGAGGGCCAAGCACTACGTGTACATCGGCGACCCCGCCCAGC | |
| | TGCCCGCCCCCAGGACCCTGCTGACCAAGGGCACCCTGGAGCCCGAG | |
| | TACTTCAACAGCGTGTGCAGGCTGATGAAGACCATCGGCCCCGACATG | |
| | TTCCTGGGCACCTGCAGGAGGTGCCCCGCCGAGATCGTGGACACCGT | |
| | GAGCGCCCTGGTGTACGACAACAAGCTGAAGGCCCACAAGGACAAGA | |
| | GCGCCCAGTGCTTCAAGATGTTCTACAAGGGCGTGATCACCCACGACG | |
| | TGAGCAGCGCCATCAACAGGCCCCAGATCGGCGTGGTGAGGGAGTTC | |
| | CTGACCAGGAACCCCGCCTGGAGGAAGGCCGTGTTCATCAGCCCCTAC | |
| | AACAGCCAGAACGCCGTGGCCAGCAAGATCCTGGGCCTGCCCACCCA | |
| | GACCGTGGACAGCAGCCAGGGCAGCGAGTACGACTACGTGATCTTCAC | |
| | CCAGACCACCGAGACCGCCCACAGCTGCAACGTGAACAGGTTCAACGT | |
| | GGCCATCACCAGGGCCAAGGTGGGCATCCTGTGCATCATGAGCGACA | |
| | GGGACCTGTACGACAAGCTGCAGTTCACCAGCCTGGAGATCCCCAGGA | |
| | GGAACGTGGCCACCCTGCAGTGATGA | |
| 28 | ATGTTCGTGTTCCTGGTGCTGCTGCCCCTGGTGAGCAGCCAGTGCGTG | 313 |
| Pan-CoV | AACCTGACCACCAGGACCCAGCTGCCCCCCGCCTACACCAACAGCTTC | |
| mRNA Spike- | ACCAGGGGCGTGTACTACCCCGACAAGGTGTTCAGGAGCAGCGTGCT | |
| NSP14- | GCACAGCACCCAGGACCTGTTCCTGCCCTTCTTCAGCAACGTGACCTG | |
| NSP15 | GTTCCACGCCATCCACGTGAGCGGCACCAACGGCACCAAGAGGTTCGA | |
| | CAACCCCGTGCTGCCCTTCAACGACGGCGTGTACTTCGCCAGCACCGA | |
| | GAAGAGCAACATCATCAGGGGCTGGATCTTCGGCACCACCCTGGACAG | |
| | CAAGACCCAGAGCCTGCTGATCGTGAACAACGCCACCAACGTGGTGAT | |
| | CAAGGTGTGCGAGTTCCAGTTCTGCAACGACCCCTTCCTGGGCGTGTA | |
| | CTACCACAAGAACAACAAGAGCTGGATGGAGAGCGAGTTCAGGGTGTA | |
| | CAGCAGCGCCAACAACTGCACCTTCGAGTACGTGAGCCAGCCCTTCCT | |
| | GATGGACCTGGAGGGCAAGCAGGGCAACTTCAAGAACCTGAGGGAGT | |
| | TCGTGTTCAAGAACATCGACGGCTACTTCAAGATCTACAGCAAGCACAC | |
| | CCCCATCAACCTGGTGAGGGACCTGCCCCAGGGCTTCAGCGCCCTGG | |
| | AGCCCCTGGTGGACCTGCCCATCGGCATCAACATCACCAGGTTCCAGA | |
| | CCCTGCTGGCCCTGCACAGGAGCTACCTGACCCCCGGCGACAGCAGC | |
| | AGCGGCTGGACCGCCGGCGCCGCCGCCTACTACGTGGGCTACCTGCA | |
| | GCCCAGGACCTTCCTGCTGAAGTACAACGAGAACGGCACCATCACCGA | |
| | CGCCGTGGACTGCGCCCTGGACCCCCTGAGCGAGACCAAGTGCACCC | |
| | TGAAGAGCTTCACCGTGGAGAAGGGCATCTACCAGACCAGCAACTTCA | |
| | GGGTGCAGCCCACCGAGAGCATCGTGAGGTTCCCCAACATCACCAACC | |
| | TGTGCCCCTTCGGCGAGGTGTTCAACGCCACCAGGTTCGCCAGCGTGT | |
| | ACGCCTGGAACAGGAAGAGGATCAGCAACTGCGTGGCCGACTACAGC | |
| | GTGCTGTACAACAGCGCCAGCTTCAGCACCTTCAAGTGCTACGGCGTG | |
| | AGCCCCACCAAGCTGAACGACCTGTGCTTCACCAACGTGTACGCCGAC | |
| | AGCTTCGTGATCAGGGGCGACGAGGTGAGGCAGATCGCCCCCGGCCA | |
| | GACCGGCAAGATCGCCGACTACAACTACAAGCTGCCCGACGACTTCAC | |
| | CGGCTGCGTGATCGCCTGGAACAGCAACAACCTGGACAGCAAGGTGG | |
| | GCGGCAACTACAACTACCTGTACAGGCTGTTCAGGAAGAGCAACCTGA | |
| | AGCCCTTCGAGAGGGACATCAGCACCGAGATCTACCAGGCCGGCAGC | |
| | ACCCCCTGCAACGGCGTGGAGGGCTTCAACTGCTACTTCCCCCTGCAG | |
| | AGCTACGGCTTCCAGCCCACCAACGGCGTGGGCTACCAGCCCTACAG | |
| | GGTGGTGGTGCTGAGCTTCGAGCTGCTGCACGCCCCCGCCACCGTGT | |
| | GCGGCCCCAAGAAGAGCACCAACCTGGTGAAGAACAAGTGCGTGAACT | |
| | TCAACTTCAACGGCCTGACCGGCACCGGCGTGCTGACCGAGAGCAACA | |
| | AGAAGTTCCTGCCCTTCCAGCAGTTCGGCAGGGACATCGCCGACACCA | |
| | CCGACGCCGTGAGGGACCCCCAGACCCTGGAGATCCTGGACATCACC | |
| | CCCTGCAGCTTCGGCGGCGTGAGCGTGATCACCCCCGGCACCAACAC | |
| | CAGCAACCAGGTGGCCGTGCTGTACCAGGACGTGAACTGCACCGAGG | |
| | TGCCCGTGGCCATCCACGCCGACCAGCTGACCCCCACCTGGAGGGTG | |
| | TACAGCACCGGCAGCAACGTGTTCCAGACCAGGGCCGGCTGCCTGAT | |
| | CGGCGCCGAGCACGTGAACAACAGCTACGAGTGCGACATCCCCATCG | |
| | GCGCCGGCATCTGCGCCAGCTACCAGACCCAGACCAACAGCCCCAGG | |
| | AGGGCCAGGAGCGTGGCCAGCCAGAGCATCATCGCCTACACCATGAG | |
| | CCTGGGCGCCGAGAACAGCGTGGCCTACAGCAACAACAGCATCGCCA | |
| | TCCCCACCAACTTCACCATCAGCGTGACCACCGAGATCCTGCCCGTGA | |
| | GCATGACCAAGACCAGCGTGGACTGCACCATGTACATCTGCGGCGACA | |
| | GCACCGAGTGCAGCAACCTGCTGCTGCAGTACGGCAGCTTCTGCACCC | |
| | AGCTGAACAGGGCCCTGACCGGCATCGCCGTGGAGCAGGACAAGAAC | |
| | ACCCAGGAGGTGTTCGCCCAGGTGAAGCAGATCTACAAGACCCCCCCC | |
| | ATCAAGGACTTCGGCGGCTTCAACTTCAGCCAGATCCTGCCCGACCCC | |
| | AGCAAGCCCAGCAAGAGGAGCTTCATCGAGGACCTGCTGTTCAACAAG | |
| | GTGACCCTGGCCGACGCCGGCTTCATCAAGCAGTACGGCGACTGCCT | |
| | GGGCGACATCGCCGCCAGGGACCTGATCTGCGCCCAGAAGTTCAACG | |
| | GCCTGACCGTGCTGCCCCCCCTGCTGACCGACGAGATGATCGCCCAG | |
| | TACACCAGCGCCCTGCTGGCCGGCACCATCACCAGCGGCTGGACCTT | |
| | CGGCGCCGGCGCCGCCCTGCAGATCCCCTTCGCCATGCAGATGGCCT | |
| | ACAGGTTCAACGGCATCGGCGTGACCCAGAACGTGCTGTACGAGAACC | |
| | AGAAGCTGATCGCCAACCAGTTCAACAGCGCCATCGGCAAGATCCAGG | |
| | ACAGCCTGAGCAGCACCGCCAGCGCCCTGGGCAAGCTGCAGGACGTG | |
| | GTGAACCAGAACGCCCAGGCCCTGAACACCCTGGTGAAGCAGCTGAG | |
| | CAGCAACTTCGGCGCCATCAGCAGCGTGCTGAACGACATCCTGAGCAG | |
| | GCTGGACCCCCCCGAGGCCGAGGTGCAGATCGACAGGCTGATCACCG | |
| | GCAGGCTGCAGAGCCTGCAGACCTACGTGACCCAGCAGCTGATCAGG | |
| | GCCGCCGAGATCAGGGCCAGCGCCAACCTGGCCGCCACCAAGATGAG | |
| | CGAGTGCGTGCTGGGCCAGAGCAAGAGGGTGGACTTCTGCGGCAAGG | |
| | GCTACCACCTGATGAGCTTCCCCCAGAGCGCCCCCCACGGCGTGGTG | |
| | TTCCTGCACGTGACCTACGTGCCCGCCCAGGAGAAGAACTTCACCACC | |
| | GCCCCCGCCATCTGCCACGACGGCAAGGCCCACTTCCCCAGGGAGGG | |
| | CGTGTTCGTGAGCAACGGCACCCACTGGTTCGTGACCCAGAGGAACTT | |
| | CTACGAGCCCCAGATCATCACCACCGACAACACCTTCGTGAGCGGCAA | |
| | CTGCGACGTGGTGATCGGCATCGTGAACAACACCGTGTACGACCCCCT | |
| | GCAGCCCGAGCTGGACAGCTTCAAGGAGGAGCTGGACAAGTACTTCAA | |
| | GAACCACACCAGCCCCGACGTGGACCTGGGCGACATCAGCGGCATCA | |
| | ACGCCAGCGTGGTGAACATCCAGAAGGAGATCGACAGGCTGAACGAG | |
| | GTGGCCAAGAACCTGAACGAGAGCCTGATCGACCTGCAGGAGCTGGG | |
| | CAAGTACGAGCAGTACATCAAGTGGCCCTGGTACATCTGGCTGGGCTT | |
| | CATCGCCGGCCTGATCGCCATCGTGATGGTGACCATCATGCTGTGCTG | |
| | CATGACCAGCTGCTGCAGCTGCCTGAAGGGCTGCTGCAGCTGCGGCA | |
| | GCTGCTGCAAGTTCGACGAGGACGACAGCGAGCCCGTGCTGAAGGGC | |
| | GTGAAGCTGCACTACACCGGAAGCGGAGCCACGAACTTCTCTCTGTTA | |
| | AAGCAAGCAGGAGATGTTGAAGAAAACCCCGGGCCTGCCGAGAACGT | |
| | GACCGGCCTGTTCAAGGACTGCAGCAAGGTGATCACCGGCCTGCACC | |
| | CCACCCAGGCCCCCACCCACCTGAGCGTGGACACCAAGTTCAAGACC | |
| | GAGGGCCTGTGCGTGGACATCCCCGGCATCCCCAAGGACATGACCTA | |
| | CAGGAGGCTGATCAGCATGATGGGCTTCAAGATGAACTACCAGGTGAA | |
| | CGGCTACCCCAACATGTTCATCACCAGGGAGGAGGCCATCAGGCACGT | |
| | GAGGGCCTGGATCGGCTTCGACGTGGAGGGCTGCCACGCCACCAGGG | |
| | AGGCCGTGGGCACCAACCTGCCCCTGCAGCTGGGCTTCAGCACCGGC | |
| | GTGAACCTGGTGGCCGTGCCCACCGGCTACGTGGACACCCCCAACAA | |
| | CACCGACTTCAGCAGGGTGAGCGCCAAGCCCCCCCCCGGCGACCAGT | |
| | TCAAGCACCTGATCCCCCTGATGTACAAGGGCCTGCCCTGGAACGTGG | |
| | TGAGGATCAAGATCGTGCAGATGCTGAGCGACACCCTGAAGAACCTGA | |
| | GCGACAGGGTGGTGTTCGTGCTGTGGGCCCACGGCTTCGAGCTGACC | |
| | AGCATGAAGTACTTCGTGAAGATCGGCCCCGAGAGGACCTGCTGCCTG | |
| | TGCGACAGGAGGGCCACCTGCTTCAGCACCGCCAGCGACACCTACGC | |
| | CTGCTGGCACCACAGCATCGGCTTCGACTACGTGTACAACCCCTTCAT | |
| | GATCGACGTGCAGCAGTGGGGCTTCACCGGCAACCTGCAGAGCAACC | |
| | ACGACCTGTACTGCCAGGTGCACGGCAACGCCCACGTGGCCAGCTGC | |
| | GACGCCATCATGACCAGGTGCCTGGCCGTGCACGAGTGCTTCGTGAAG | |
| | AGGGTGGACTGGACCATCGAGTACCCCATCATCGGCGACGAGCTGAA | |
| | GATCAACGCCGCCTGCAGGAAGGTGCAGCACATGGTGGTGAAGGCCG | |
| | CCCTGCTGGCCGACAAGTTCCCCGTGCTGCACGACATCGGCAACCCCA | |
| | AGGCCATCAAGTGCGTGCCCCAGGCCGACGTGGAGTGGAAGTTCTAC | |
| | GACGCCCAGCCCTGCAGCGACAAGGCCTACAAGATCGAGGAGCTGTT | |
| | CTACAGCTACGCCACCCACAGCGACAAGTTCACCGACGGCGTGTGCCT | |
| | GTTCTGGAACTGCAACGTGGACAGGTACCCCGCCAACAGCATCGTGTG | |
| | CAGGTTCGACACCAGGGTGCTGAGCAACCTGAACCTGCCCGGCTGCG | |
| | ACGGCGGCAGCCTGTACGTGAACAAGCACGCCTTCCACACCCCCGCCT | |
| | TCGACAAGAGCGCCTTCGTGAACCTGAAGCAGCTGCCCTTCTTCTACTA | |
| | CAGCGACAGCCCCTGCGAGAGCCACGGCAAGCAGGTGGTGAGCGACA | |
| | TCGACTACGTGCCCCTGAAGAGCGCCACCTGCATCACCAGGTGCAACC | |
| | TGGGCGGCGCCGTGTGCAGGCACCACGCCAACGAGTACAGGCTGTAC | |
| | CTGGACGCCTACAACATGATGATCAGCGCCGGCTTCAGCCTGTGGGTG | |
| | TACAAGCAGTTCGACACCTACAACCTGTGGAACACCTTCACCAGGCTG | |
| | CAGGGAAGCGGAGAGGGCAGGGGAAGTCTTCTAACATGCGGGGACGT | |
| | GGAGGAAAATCCCGGCCCCAGCCTGGAGAACGTGGCCTTCAACGTGG | |
| | TGAACAAGGGCCACTTCGACGGCCAGCAGGGCGAGGTGCCCGTGAGC | |
| | ATCATCAACAACACCGTGTACACCAAGGTGGACGGCGTGGACGTGGAG | |
| | CTGTTCGAGAACAAGACCACCCTGCCCGTGAACGTGGCCTTCGAGCTG | |
| | TGGGCCAAGAGGAACATCAAGCCCGTGCCCGAGGTGAAGATCCTGAAC | |
| | AACCTGGGCGTGGACATCGCCGCCAACACCGTGATCTGGGACTACAAG | |
| | AGGGACGCCCCCGCCCACATCAGCACCATCGGCGTGTGCAGCATGAC | |
| | CGACATCGCCAAGAAGCCCACCGAGACCATCTGCGCCCCCCTGACCGT | |
| | GTTCTTCGACGGCAGGGTGGACGGCCAGGTGGACCTGTTCAGGAACG | |
| | CCAGGAACGGCGTGCTGATCACCGAGGGCAGCGTGAAGGGCCTGCAG | |
| | CCCAGCGTGGGCCCCAAGCAGGCCAGCCTGAACGGCGTGACCCTGAT | |
| | CGGCGAGGCCGTGAAGACCCAGTTCAACTACTACAAGAAGGTGGACG | |
| | GCGTGGTGCAGCAGCTGCCCGAGACCTACTTCACCCAGAGCAGGAAC | |
| | CTGCAGGAGTTCAAGCCCAGGAGCCAGATGGAGATCGACTTCCTGGAG | |
| | CTGGCCATGGACGAGTTCATCGAGAGGTACAAGCTGGAGGGCTACGC | |
| | CTTCGAGCACATCGTGTACGGCGACTTCAGCCACAGCCAGCTGGGCG | |
| | GCCTGCACCTGCTGATCGGCCTGGCCAAGAGGTTCAAGGAGAGCCCC | |
| | TTCGAGCTGGAGGACTTCATCCCCATGGACAGCACCGTGAAGAACTAC | |
| | TTCATCACCGACGCCCAGACCGGCAGCAGCAAGTGCGTGTGCAGCGT | |
| | GATCGACCTGCTGCTGGACGACTTCGTGGAGATCATCAAGAGCCAGGA | |
| | CCTGAGCGTGGTGAGCAAGGTGGTGAAGGTGACCATCGACTACACCGA | |
| | GATCAGCTTCATGCTGTGGTGCAAGGACGGCCACGTGGAGACCTTCTA | |
| | CCCCAAGCTGCAGTGATGA | |
| 29 | ATGTTCGTGTTCCTGGTGCTGCTGCCCCTGGTGAGCAGCCAGTGCGTG | 314 |
| Pan-CoV | AACCTGACCACCAGGACCCAGCTGCCCCCCGCCTACACCAACAGCTTC | |
| mRNA | ACCAGGGGCGTGTACTACCCCGACAAGGTGTTCAGGAGCAGCGTGCT | |
| HexaPro- | GCACAGCACCCAGGACCTGTTCCTGCCCTTCTTCAGCAACGTGACCTG | |
| NSP14- | GTTCCACGCCATCCACGTGAGCGGCACCAACGGCACCAAGAGGTTCGA | |
| NSP15 | CAACCCCGTGCTGCCCTTCAACGACGGCGTGTACTTCGCCAGCACCGA | |
| | GAAGAGCAACATCATCAGGGGCTGGATCTTCGGCACCACCCTGGACAG | |
| | CAAGACCCAGAGCCTGCTGATCGTGAACAACGCCACCAACGTGGTGAT | |
| | CAAGGTGTGCGAGTTCCAGTTCTGCAACGACCCCTTCCTGGGCGTGTA | |
| | CTACCACAAGAACAACAAGAGCTGGATGGAGAGCGAGTTCAGGGTGTA | |
| | CAGCAGCGCCAACAACTGCACCTTCGAGTACGTGAGCCAGCCCTTCCT | |
| | GATGGACCTGGAGGGCAAGCAGGGCAACTTCAAGAACCTGAGGGAGT | |
| | TCGTGTTCAAGAACATCGACGGCTACTTCAAGATCTACAGCAAGCACAC | |
| | CCCCATCAACCTGGTGAGGGACCTGCCCCAGGGCTTCAGCGCCCTGG | |
| | AGCCCCTGGTGGACCTGCCCATCGGCATCAACATCACCAGGTTCCAGA | |
| | CCCTGCTGGCCCTGCACAGGAGCTACCTGACCCCCGGCGACAGCAGC | |
| | AGCGGCTGGACCGCCGGCGCCGCCGCCTACTACGTGGGCTACCTGCA | |
| | GCCCAGGACCTTCCTGCTGAAGTACAACGAGAACGGCACCATCACCGA | |
| | CGCCGTGGACTGCGCCCTGGACCCCCTGAGCGAGACCAAGTGCACCC | |
| | TGAAGAGCTTCACCGTGGAGAAGGGCATCTACCAGACCAGCAACTTCA | |
| | GGGTGCAGCCCACCGAGAGCATCGTGAGGTTCCCCAACATCACCAACC | |
| | TGTGCCCCTTCGGCGAGGTGTTCAACGCCACCAGGTTCGCCAGCGTGT | |
| | ACGCCTGGAACAGGAAGAGGATCAGCAACTGCGTGGCCGACTACAGC | |
| | GTGCTGTACAACAGCGCCAGCTTCAGCACCTTCAAGTGCTACGGCGTG | |
| | AGCCCCACCAAGCTGAACGACCTGTGCTTCACCAACGTGTACGCCGAC | |
| | AGCTTCGTGATCAGGGGCGACGAGGTGAGGCAGATCGCCCCCGGCCA | |
| | GACCGGCAAGATCGCCGACTACAACTACAAGCTGCCCGACGACTTCAC | |
| | CGGCTGCGTGATCGCCTGGAACAGCAACAACCTGGACAGCAAGGTGG | |
| | GCGGCAACTACAACTACCTGTACAGGCTGTTCAGGAAGAGCAACCTGA | |
| | AGCCCTTCGAGAGGGACATCAGCACCGAGATCTACCAGGCCGGCAGC | |
| | ACCCCCTGCAACGGCGTGGAGGGCTTCAACTGCTACTTCCCCCTGCAG | |
| | AGCTACGGCTTCCAGCCCACCAACGGCGTGGGCTACCAGCCCTACAG | |
| | GGTGGTGGTGCTGAGCTTCGAGCTGCTGCACGCCCCCGCCACCGTGT | |
| | GCGGCCCCAAGAAGAGCACCAACCTGGTGAAGAACAAGTGCGTGAACT | |
| | TCAACTTCAACGGCCTGACCGGCACCGGCGTGCTGACCGAGAGCAACA | |
| | AGAAGTTCCTGCCCTTCCAGCAGTTCGGCAGGGACATCGCCGACACCA | |
| | CCGACGCCGTGAGGGACCCCCAGACCCTGGAGATCCTGGACATCACC | |
| | CCCTGCAGCTTCGGCGGCGTGAGCGTGATCACCCCCGGCACCAACAC | |
| | CAGCAACCAGGTGGCCGTGCTGTACCAGGACGTGAACTGCACCGAGG | |
| | TGCCCGTGGCCATCCACGCCGACCAGCTGACCCCCACCTGGAGGGTG | |
| | TACAGCACCGGCAGCAACGTGTTCCAGACCAGGGCCGGCTGCCTGAT | |
| | CGGCGCCGAGCACGTGAACAACAGCTACGAGTGCGACATCCCCATCG | |
| | GCGCCGGCATCTGCGCCAGCTACCAGACCCAGACCAACAGCCCCGGC | |
| | AGCGCCAGCAGCGTGGCCAGCCAGAGCATCATCGCCTACACCATGAG | |
| | CCTGGGCGCCGAGAACAGCGTGGCCTACAGCAACAACAGCATCGCCA | |
| | TCCCCACCAACTTCACCATCAGCGTGACCACCGAGATCCTGCCCGTGA | |
| | GCATGACCAAGACCAGCGTGGACTGCACCATGTACATCTGCGGCGACA | |
| | GCACCGAGTGCAGCAACCTGCTGCTGCAGTACGGCAGCTTCTGCACCC | |
| | AGCTGAACAGGGCCCTGACCGGCATCGCCGTGGAGCAGGACAAGAAC | |
| | ACCCAGGAGGTGTTCGCCCAGGTGAAGCAGATCTACAAGACCCCCCCC | |
| | ATCAAGGACTTCGGCGGCTTCAACTTCAGCCAGATCCTGCCCGACCCC | |
| | AGCAAGCCCAGCAAGAGGAGCCCCATCGAGGACCTGCTGTTCAACAAG | |
| | GTGACCCTGGCCGACGCCGGCTTCATCAAGCAGTACGGCGACTGCCT | |
| | GGGCGACATCGCCGCCAGGGACCTGATCTGCGCCCAGAAGTTCAACG | |
| | GCCTGACCGTGCTGCCCCCCCTGCTGACCGACGAGATGATCGCCCAG | |
| | TACACCAGCGCCCTGCTGGCCGGCACCATCACCAGCGGCTGGACCTT | |
| | CGGCGCCGGCCCCGCCCTGCAGATCCCCTTCCCCATGCAGATGGCCT | |
| | ACAGGTTCAACGGCATCGGCGTGACCCAGAACGTGCTGTACGAGAACC | |
| | AGAAGCTGATCGCCAACCAGTTCAACAGCGCCATCGGCAAGATCCAGG | |
| | ACAGCCTGAGCAGCACCCCCAGCGCCCTGGGCAAGCTGCAGGACGTG | |
| | GTGAACCAGAACGCCCAGGCCCTGAACACCCTGGTGAAGCAGCTGAG | |
| | CAGCAACTTCGGCGCCATCAGCAGCGTGCTGAACGACATCCTGAGCAG | |
| | GCTGGACCCCCCCGAGGCCGAGGTGCAGATCGACAGGCTGATCACCG | |
| | GCAGGCTGCAGAGCCTGCAGACCTACGTGACCCAGCAGCTGATCAGG | |
| | GCCGCCGAGATCAGGGCCAGCGCCAACCTGGCCGCCACCAAGATGAG | |
| | CGAGTGCGTGCTGGGCCAGAGCAAGAGGGTGGACTTCTGCGGCAAGG | |
| | GCTACCACCTGATGAGCTTCCCCCAGAGCGCCCCCCACGGCGTGGTG | |
| | TTCCTGCACGTGACCTACGTGCCCGCCCAGGAGAAGAACTTCACCACC | |
| | GCCCCCGCCATCTGCCACGACGGCAAGGCCCACTTCCCCAGGGAGGG | |
| | CGTGTTCGTGAGCAACGGCACCCACTGGTTCGTGACCCAGAGGAACTT | |
| | CTACGAGCCCCAGATCATCACCACCGACAACACCTTCGTGAGCGGCAA | |
| | CTGCGACGTGGTGATCGGCATCGTGAACAACACCGTGTACGACCCCCT | |
| | GCAGCCCGAGCTGGACAGCTTCAAGGAGGAGCTGGACAAGTACTTCAA | |
| | GAACCACACCAGCCCCGACGTGGACCTGGGCGACATCAGCGGCATCA | |
| | ACGCCAGCGTGGTGAACATCCAGAAGGAGATCGACAGGCTGAACGAG | |
| | GTGGCCAAGAACCTGAACGAGAGCCTGATCGACCTGCAGGAGCTGGG | |
| | CAAGTACGAGCAGTACATCAAGTGGCCCTGGTACATCTGGCTGGGCTT | |
| | CATCGCCGGCCTGATCGCCATCGTGATGGTGACCATCATGCTGTGCTG | |
| | CATGACCAGCTGCTGCAGCTGCCTGAAGGGCTGCTGCAGCTGCGGCA | |
| | GCTGCTGCAAGTTCGACGAGGACGACAGCGAGCCCGTGCTGAAGGGC | |
| | GTGAAGCTGCACTACACCGGAAGCGGAGCCACGAACTTCTCTCTGTTA | |
| | AAGCAAGCAGGAGATGTTGAAGAAAACCCCGGGCCTGCCGAGAACGT | |
| | GACCGGCCTGTTCAAGGACTGCAGCAAGGTGATCACCGGCCTGCACC | |
| | CCACCCAGGCCCCCACCCACCTGAGCGTGGACACCAAGTTCAAGACC | |
| | GAGGGCCTGTGCGTGGACATCCCCGGCATCCCCAAGGACATGACCTA | |
| | CAGGAGGCTGATCAGCATGATGGGCTTCAAGATGAACTACCAGGTGAA | |
| | CGGCTACCCCAACATGTTCATCACCAGGGAGGAGGCCATCAGGCACGT | |
| | GAGGGCCTGGATCGGCTTCGACGTGGAGGGCTGCCACGCCACCAGGG | |
| | AGGCCGTGGGCACCAACCTGCCCCTGCAGCTGGGCTTCAGCACCGGC | |
| | GTGAACCTGGTGGCCGTGCCCACCGGCTACGTGGACACCCCCAACAA | |
| | CACCGACTTCAGCAGGGTGAGCGCCAAGCCCCCCCCCGGCGACCAGT | |
| | TCAAGCACCTGATCCCCCTGATGTACAAGGGCCTGCCCTGGAACGTGG | |
| | TGAGGATCAAGATCGTGCAGATGCTGAGCGACACCCTGAAGAACCTGA | |
| | GCGACAGGGTGGTGTTCGTGCTGTGGGCCCACGGCTTCGAGCTGACC | |
| | AGCATGAAGTACTTCGTGAAGATCGGCCCCGAGAGGACCTGCTGCCTG | |
| | TGCGACAGGAGGGCCACCTGCTTCAGCACCGCCAGCGACACCTACGC | |
| | CTGCTGGCACCACAGCATCGGCTTCGACTACGTGTACAACCCCTTCAT | |
| | GATCGACGTGCAGCAGTGGGGCTTCACCGGCAACCTGCAGAGCAACC | |
| | ACGACCTGTACTGCCAGGTGCACGGCAACGCCCACGTGGCCAGCTGC | |
| | GACGCCATCATGACCAGGTGCCTGGCCGTGCACGAGTGCTTCGTGAAG | |
| | AGGGTGGACTGGACCATCGAGTACCCCATCATCGGCGACGAGCTGAA | |
| | GATCAACGCCGCCTGCAGGAAGGTGCAGCACATGGTGGTGAAGGCCG | |
| | CCCTGCTGGCCGACAAGTTCCCCGTGCTGCACGACATCGGCAACCCCA | |
| | AGGCCATCAAGTGCGTGCCCCAGGCCGACGTGGAGTGGAAGTTCTAC | |
| | GACGCCCAGCCCTGCAGCGACAAGGCCTACAAGATCGAGGAGCTGTT | |
| | CTACAGCTACGCCACCCACAGCGACAAGTTCACCGACGGCGTGTGCCT | |
| | GTTCTGGAACTGCAACGTGGACAGGTACCCCGCCAACAGCATCGTGTG | |
| | CAGGTTCGACACCAGGGTGCTGAGCAACCTGAACCTGCCCGGCTGCG | |
| | ACGGCGGCAGCCTGTACGTGAACAAGCACGCCTTCCACACCCCCGCCT | |
| | TCGACAAGAGCGCCTTCGTGAACCTGAAGCAGCTGCCCTTCTTCTACTA | |
| | CAGCGACAGCCCCTGCGAGAGCCACGGCAAGCAGGTGGTGAGCGACA | |
| | TCGACTACGTGCCCCTGAAGAGCGCCACCTGCATCACCAGGTGCAACC | |
| | TGGGCGGCGCCGTGTGCAGGCACCACGCCAACGAGTACAGGCTGTAC | |
| | CTGGACGCCTACAACATGATGATCAGCGCCGGCTTCAGCCTGTGGGTG | |
| | TACAAGCAGTTCGACACCTACAACCTGTGGAACACCTTCACCAGGCTG | |
| | CAGGGAAGCGGAGAGGGCAGGGGAAGTCTTCTAACATGCGGGGACGT | |
| | GGAGGAAAATCCCGGCCCCAGCCTGGAGAACGTGGCCTTCAACGTGG | |
| | TGAACAAGGGCCACTTCGACGGCCAGCAGGGCGAGGTGCCCGTGAGC | |
| | ATCATCAACAACACCGTGTACACCAAGGTGGACGGCGTGGACGTGGAG | |
| | CTGTTCGAGAACAAGACCACCCTGCCCGTGAACGTGGCCTTCGAGCTG | |
| | TGGGCCAAGAGGAACATCAAGCCCGTGCCCGAGGTGAAGATCCTGAAC | |
| | AACCTGGGCGTGGACATCGCCGCCAACACCGTGATCTGGGACTACAAG | |
| | AGGGACGCCCCCGCCCACATCAGCACCATCGGCGTGTGCAGCATGAC | |
| | CGACATCGCCAAGAAGCCCACCGAGACCATCTGCGCCCCCCTGACCGT | |
| | GTTCTTCGACGGCAGGGTGGACGGCCAGGTGGACCTGTTCAGGAACG | |
| | CCAGGAACGGCGTGCTGATCACCGAGGGCAGCGTGAAGGGCCTGCAG | |
| | CCCAGCGTGGGCCCCAAGCAGGCCAGCCTGAACGGCGTGACCCTGAT | |
| | CGGCGAGGCCGTGAAGACCCAGTTCAACTACTACAAGAAGGTGGACG | |
| | GCGTGGTGCAGCAGCTGCCCGAGACCTACTTCACCCAGAGCAGGAAC | |
| | CTGCAGGAGTTCAAGCCCAGGAGCCAGATGGAGATCGACTTCCTGGAG | |
| | CTGGCCATGGACGAGTTCATCGAGAGGTACAAGCTGGAGGGCTACGC | |
| | CTTCGAGCACATCGTGTACGGCGACTTCAGCCACAGCCAGCTGGGCG | |
| | GCCTGCACCTGCTGATCGGCCTGGCCAAGAGGTTCAAGGAGAGCCCC | |
| | TTCGAGCTGGAGGACTTCATCCCCATGGACAGCACCGTGAAGAACTAC | |
| | TTCATCACCGACGCCCAGACCGGCAGCAGCAAGTGCGTGTGCAGCGT | |
| | GATCGACCTGCTGCTGGACGACTTCGTGGAGATCATCAAGAGCCAGGA | |
| | CCTGAGCGTGGTGAGCAAGGTGGTGAAGGTGACCATCGACTACACCGA | |
| | GATCAGCTTCATGCTGTGGTGCAAGGACGGCCACGTGGAGACCTTCTA | |
| | CCCCAAGCTGCAGTGATGA | |
| 30 | ATGTTCGTGTTCCTGGTGCTGCTGCCCCTGGTGAGCAGCCAGTGCGTG | 315 |
| Pan-CoV | AACCTGACCACCAGGACCCAGCTGCCCCCCGCCTACACCAACAGCTTC | |
| mRNA Spike- | ACCAGGGGCGTGTACTACCCCGACAAGGTGTTCAGGAGCAGCGTGCT | |
| NSP16 | GCACAGCACCCAGGACCTGTTCCTGCCCTTCTTCAGCAACGTGACCTG | |
| | GTTCCACGCCATCCACGTGAGCGGCACCAACGGCACCAAGAGGTTCGA | |
| | CAACCCCGTGCTGCCCTTCAACGACGGCGTGTACTTCGCCAGCACCGA | |
| | GAAGAGCAACATCATCAGGGGCTGGATCTTCGGCACCACCCTGGACAG | |
| | CAAGACCCAGAGCCTGCTGATCGTGAACAACGCCACCAACGTGGTGAT | |
| | CAAGGTGTGCGAGTTCCAGTTCTGCAACGACCCCTTCCTGGGCGTGTA | |
| | CTACCACAAGAACAACAAGAGCTGGATGGAGAGCGAGTTCAGGGTGTA | |
| | CAGCAGCGCCAACAACTGCACCTTCGAGTACGTGAGCCAGCCCTTCCT | |
| | GATGGACCTGGAGGGCAAGCAGGGCAACTTCAAGAACCTGAGGGAGT | |
| | TCGTGTTCAAGAACATCGACGGCTACTTCAAGATCTACAGCAAGCACAC | |
| | CCCCATCAACCTGGTGAGGGACCTGCCCCAGGGCTTCAGCGCCCTGG | |
| | AGCCCCTGGTGGACCTGCCCATCGGCATCAACATCACCAGGTTCCAGA | |
| | CCCTGCTGGCCCTGCACAGGAGCTACCTGACCCCCGGCGACAGCAGC | |
| | AGCGGCTGGACCGCCGGCGCCGCCGCCTACTACGTGGGCTACCTGCA | |
| | GCCCAGGACCTTCCTGCTGAAGTACAACGAGAACGGCACCATCACCGA | |
| | CGCCGTGGACTGCGCCCTGGACCCCCTGAGCGAGACCAAGTGCACCC | |
| | TGAAGAGCTTCACCGTGGAGAAGGGCATCTACCAGACCAGCAACTTCA | |
| | GGGTGCAGCCCACCGAGAGCATCGTGAGGTTCCCCAACATCACCAACC | |
| | TGTGCCCCTTCGGCGAGGTGTTCAACGCCACCAGGTTCGCCAGCGTGT | |
| | ACGCCTGGAACAGGAAGAGGATCAGCAACTGCGTGGCCGACTACAGC | |
| | GTGCTGTACAACAGCGCCAGCTTCAGCACCTTCAAGTGCTACGGCGTG | |
| | AGCCCCACCAAGCTGAACGACCTGTGCTTCACCAACGTGTACGCCGAC | |
| | AGCTTCGTGATCAGGGGCGACGAGGTGAGGCAGATCGCCCCCGGCCA | |
| | GACCGGCAAGATCGCCGACTACAACTACAAGCTGCCCGACGACTTCAC | |
| | CGGCTGCGTGATCGCCTGGAACAGCAACAACCTGGACAGCAAGGTGG | |
| | GCGGCAACTACAACTACCTGTACAGGCTGTTCAGGAAGAGCAACCTGA | |
| | AGCCCTTCGAGAGGGACATCAGCACCGAGATCTACCAGGCCGGCAGC | |
| | ACCCCCTGCAACGGCGTGGAGGGCTTCAACTGCTACTTCCCCCTGCAG | |
| | AGCTACGGCTTCCAGCCCACCAACGGCGTGGGCTACCAGCCCTACAG | |
| | GGTGGTGGTGCTGAGCTTCGAGCTGCTGCACGCCCCCGCCACCGTGT | |
| | GCGGCCCCAAGAAGAGCACCAACCTGGTGAAGAACAAGTGCGTGAACT | |
| | TCAACTTCAACGGCCTGACCGGCACCGGCGTGCTGACCGAGAGCAACA | |
| | AGAAGTTCCTGCCCTTCCAGCAGTTCGGCAGGGACATCGCCGACACCA | |
| | CCGACGCCGTGAGGGACCCCCAGACCCTGGAGATCCTGGACATCACC | |
| | CCCTGCAGCTTCGGCGGCGTGAGCGTGATCACCCCCGGCACCAACAC | |
| | CAGCAACCAGGTGGCCGTGCTGTACCAGGACGTGAACTGCACCGAGG | |
| | TGCCCGTGGCCATCCACGCCGACCAGCTGACCCCCACCTGGAGGGTG | |
| | TACAGCACCGGCAGCAACGTGTTCCAGACCAGGGCCGGCTGCCTGAT | |
| | CGGCGCCGAGCACGTGAACAACAGCTACGAGTGCGACATCCCCATCG | |
| | GCGCCGGCATCTGCGCCAGCTACCAGACCCAGACCAACAGCCCCAGG | |
| | AGGGCCAGGAGCGTGGCCAGCCAGAGCATCATCGCCTACACCATGAG | |
| | CCTGGGCGCCGAGAACAGCGTGGCCTACAGCAACAACAGCATCGCCA | |
| | TCCCCACCAACTTCACCATCAGCGTGACCACCGAGATCCTGCCCGTGA | |
| | GCATGACCAAGACCAGCGTGGACTGCACCATGTACATCTGCGGCGACA | |
| | GCACCGAGTGCAGCAACCTGCTGCTGCAGTACGGCAGCTTCTGCACCC | |
| | AGCTGAACAGGGCCCTGACCGGCATCGCCGTGGAGCAGGACAAGAAC | |
| | ACCCAGGAGGTGTTCGCCCAGGTGAAGCAGATCTACAAGACCCCCCCC | |
| | ATCAAGGACTTCGGCGGCTTCAACTTCAGCCAGATCCTGCCCGACCCC | |
| | AGCAAGCCCAGCAAGAGGAGCTTCATCGAGGACCTGCTGTTCAACAAG | |
| | GTGACCCTGGCCGACGCCGGCTTCATCAAGCAGTACGGCGACTGCCT | |
| | GGGCGACATCGCCGCCAGGGACCTGATCTGCGCCCAGAAGTTCAACG | |
| | GCCTGACCGTGCTGCCCCCCCTGCTGACCGACGAGATGATCGCCCAG | |
| | TACACCAGCGCCCTGCTGGCCGGCACCATCACCAGCGGCTGGACCTT | |
| | CGGCGCCGGCGCCGCCCTGCAGATCCCCTTCGCCATGCAGATGGCCT | |
| | ACAGGTTCAACGGCATCGGCGTGACCCAGAACGTGCTGTACGAGAACC | |
| | AGAAGCTGATCGCCAACCAGTTCAACAGCGCCATCGGCAAGATCCAGG | |
| | ACAGCCTGAGCAGCACCGCCAGCGCCCTGGGCAAGCTGCAGGACGTG | |
| | GTGAACCAGAACGCCCAGGCCCTGAACACCCTGGTGAAGCAGCTGAG | |
| | CAGCAACTTCGGCGCCATCAGCAGCGTGCTGAACGACATCCTGAGCAG | |
| | GCTGGACCCCCCCGAGGCCGAGGTGCAGATCGACAGGCTGATCACCG | |
| | GCAGGCTGCAGAGCCTGCAGACCTACGTGACCCAGCAGCTGATCAGG | |
| | GCCGCCGAGATCAGGGCCAGCGCCAACCTGGCCGCCACCAAGATGAG | |
| | CGAGTGCGTGCTGGGCCAGAGCAAGAGGGTGGACTTCTGCGGCAAGG | |
| | GCTACCACCTGATGAGCTTCCCCCAGAGCGCCCCCCACGGCGTGGTG | |
| | TTCCTGCACGTGACCTACGTGCCCGCCCAGGAGAAGAACTTCACCACC | |
| | GCCCCCGCCATCTGCCACGACGGCAAGGCCCACTTCCCCAGGGAGGG | |
| | CGTGTTCGTGAGCAACGGCACCCACTGGTTCGTGACCCAGAGGAACTT | |
| | CTACGAGCCCCAGATCATCACCACCGACAACACCTTCGTGAGCGGCAA | |
| | CTGCGACGTGGTGATCGGCATCGTGAACAACACCGTGTACGACCCCCT | |
| | GCAGCCCGAGCTGGACAGCTTCAAGGAGGAGCTGGACAAGTACTTCAA | |
| | GAACCACACCAGCCCCGACGTGGACCTGGGCGACATCAGCGGCATCA | |
| | ACGCCAGCGTGGTGAACATCCAGAAGGAGATCGACAGGCTGAACGAG | |
| | GTGGCCAAGAACCTGAACGAGAGCCTGATCGACCTGCAGGAGCTGGG | |
| | CAAGTACGAGCAGTACATCAAGTGGCCCTGGTACATCTGGCTGGGCTT | |
| | CATCGCCGGCCTGATCGCCATCGTGATGGTGACCATCATGCTGTGCTG | |
| | CATGACCAGCTGCTGCAGCTGCCTGAAGGGCTGCTGCAGCTGCGGCA | |
| | GCTGCTGCAAGTTCGACGAGGACGACAGCGAGCCCGTGCTGAAGGGC | |
| | GTGAAGCTGCACTACACCGGAAGCGGAGCCACGAACTTCTCTCTGTTA | |
| | AAGCAAGCAGGAGATGTTGAAGAAAACCCCGGGCCTAGCAGCCAGGC | |
| | CTGGCAGCCCGGCGTGGCCATGCCCAACCTGTACAAGATGCAGAGGA | |
| | TGCTGCTGGAGAAGTGCGACCTGCAGAACTACGGCGACAGCGCCACC | |
| | CTGCCCAAGGGCATCATGATGAACGTGGCCAAGTACACCCAGCTGTGC | |
| | CAGTACCTGAACACCCTGACCCTGGCCGTGCCCTACAACATGAGGGTG | |
| | ATCCACTTCGGCGCCGGCAGCGACAAGGGCGTGGCCCCCGGCACCGC | |
| | CGTGCTGAGGCAGTGGCTGCCCACCGGCACCCTGCTGGTGGACAGCG | |
| | ACCTGAACGACTTCGTGAGCGACGCCGACAGCACCCTGATCGGCGACT | |
| | GCGCCACCGTGCACACCGCCAACAAGTGGGACCTGATCATCAGCGACA | |
| | TGTACGACCCCAAGACCAAGAACGTGACCAAGGAGAACGACAGCAAGG | |
| | AGGGCTTCTTCACCTACATCTGCGGCTTCATCCAGCAGAAGCTGGCCC | |
| | TGGGCGGCAGCGTGGCCATCAAGATCACCGAGCACAGCTGGAACGCC | |
| | GACCTGTACAAGCTGATGGGCCACTTCGCCTGGTGGACCGCCTTCGTG | |
| | ACCAACGTGAACGCCAGCAGCAGCGAGGCCTTCCTGATCGGCTGCAAC | |
| | TACCTGGGCAAGCCCAGGGAGCAGATCGACGGCTACGTGATGCACGC | |
| | CAACTACATCTTCTGGAGGAACACCAACCCCATCCAGCTGAGCAGCTA | |
| | CAGCCTGTTCGACATGAGCAAGTTCCCCCTGAAGCTGAGGGGCACCGC | |
| | CGTGATGAGCCTGAAGGAGGGCCAGATCAACGACATGATCCTGAGCCT | |
| | GCTGAGCAAGGGCAGGCTGATCATCAGGGAGAACAACAGGGTGGTGA | |
| | TCAGCAGCGACGTGCTGGTGAACAACTGATGA | |
| 31 | ATGTTCGTGTTCCTGGTGCTGCTGCCCCTGGTGAGCAGCCAGTGCGTG | 316 |
| Pan-CoV | AACCTGACCACCAGGACCCAGCTGCCCCCCGCCTACACCAACAGCTTC | |
| mRNA | ACCAGGGGCGTGTACTACCCCGACAAGGTGTTCAGGAGCAGCGTGCT | |
| HexaPro- | GCACAGCACCCAGGACCTGTTCCTGCCCTTCTTCAGCAACGTGACCTG | |
| NSP16 | GTTCCACGCCATCCACGTGAGCGGCACCAACGGCACCAAGAGGTTCGA | |
| | CAACCCCGTGCTGCCCTTCAACGACGGCGTGTACTTCGCCAGCACCGA | |
| | GAAGAGCAACATCATCAGGGGCTGGATCTTCGGCACCACCCTGGACAG | |
| | CAAGACCCAGAGCCTGCTGATCGTGAACAACGCCACCAACGTGGTGAT | |
| | CAAGGTGTGCGAGTTCCAGTTCTGCAACGACCCCTTCCTGGGCGTGTA | |
| | CTACCACAAGAACAACAAGAGCTGGATGGAGAGCGAGTTCAGGGTGTA | |
| | CAGCAGCGCCAACAACTGCACCTTCGAGTACGTGAGCCAGCCCTTCCT | |
| | GATGGACCTGGAGGGCAAGCAGGGCAACTTCAAGAACCTGAGGGAGT | |
| | TCGTGTTCAAGAACATCGACGGCTACTTCAAGATCTACAGCAAGCACAC | |
| | CCCCATCAACCTGGTGAGGGACCTGCCCCAGGGCTTCAGCGCCCTGG | |
| | AGCCCCTGGTGGACCTGCCCATCGGCATCAACATCACCAGGTTCCAGA | |
| | CCCTGCTGGCCCTGCACAGGAGCTACCTGACCCCCGGCGACAGCAGC | |
| | AGCGGCTGGACCGCCGGCGCCGCCGCCTACTACGTGGGCTACCTGCA | |
| | GCCCAGGACCTTCCTGCTGAAGTACAACGAGAACGGCACCATCACCGA | |
| | CGCCGTGGACTGCGCCCTGGACCCCCTGAGCGAGACCAAGTGCACCC | |
| | TGAAGAGCTTCACCGTGGAGAAGGGCATCTACCAGACCAGCAACTTCA | |
| | GGGTGCAGCCCACCGAGAGCATCGTGAGGTTCCCCAACATCACCAACC | |
| | TGTGCCCCTTCGGCGAGGTGTTCAACGCCACCAGGTTCGCCAGCGTGT | |
| | ACGCCTGGAACAGGAAGAGGATCAGCAACTGCGTGGCCGACTACAGC | |
| | GTGCTGTACAACAGCGCCAGCTTCAGCACCTTCAAGTGCTACGGCGTG | |
| | AGCCCCACCAAGCTGAACGACCTGTGCTTCACCAACGTGTACGCCGAC | |
| | AGCTTCGTGATCAGGGGCGACGAGGTGAGGCAGATCGCCCCCGGCCA | |
| | GACCGGCAAGATCGCCGACTACAACTACAAGCTGCCCGACGACTTCAC | |
| | CGGCTGCGTGATCGCCTGGAACAGCAACAACCTGGACAGCAAGGTGG | |
| | GCGGCAACTACAACTACCTGTACAGGCTGTTCAGGAAGAGCAACCTGA | |
| | AGCCCTTCGAGAGGGACATCAGCACCGAGATCTACCAGGCCGGCAGC | |
| | ACCCCCTGCAACGGCGTGGAGGGCTTCAACTGCTACTTCCCCCTGCAG | |
| | AGCTACGGCTTCCAGCCCACCAACGGCGTGGGCTACCAGCCCTACAG | |
| | GGTGGTGGTGCTGAGCTTCGAGCTGCTGCACGCCCCCGCCACCGTGT | |
| | GCGGCCCCAAGAAGAGCACCAACCTGGTGAAGAACAAGTGCGTGAACT | |
| | TCAACTTCAACGGCCTGACCGGCACCGGCGTGCTGACCGAGAGCAACA | |
| | AGAAGTTCCTGCCCTTCCAGCAGTTCGGCAGGGACATCGCCGACACCA | |
| | CCGACGCCGTGAGGGACCCCCAGACCCTGGAGATCCTGGACATCACC | |
| | CCCTGCAGCTTCGGCGGCGTGAGCGTGATCACCCCCGGCACCAACAC | |
| | CAGCAACCAGGTGGCCGTGCTGTACCAGGACGTGAACTGCACCGAGG | |
| | TGCCCGTGGCCATCCACGCCGACCAGCTGACCCCCACCTGGAGGGTG | |
| | TACAGCACCGGCAGCAACGTGTTCCAGACCAGGGCCGGCTGCCTGAT | |
| | CGGCGCCGAGCACGTGAACAACAGCTACGAGTGCGACATCCCCATCG | |
| | GCGCCGGCATCTGCGCCAGCTACCAGACCCAGACCAACAGCCCCGGC | |
| | AGCGCCAGCAGCGTGGCCAGCCAGAGCATCATCGCCTACACCATGAG | |
| | CCTGGGCGCCGAGAACAGCGTGGCCTACAGCAACAACAGCATCGCCA | |
| | TCCCCACCAACTTCACCATCAGCGTGACCACCGAGATCCTGCCCGTGA | |
| | GCATGACCAAGACCAGCGTGGACTGCACCATGTACATCTGCGGCGACA | |
| | GCACCGAGTGCAGCAACCTGCTGCTGCAGTACGGCAGCTTCTGCACCC | |
| | AGCTGAACAGGGCCCTGACCGGCATCGCCGTGGAGCAGGACAAGAAC | |
| | ACCCAGGAGGTGTTCGCCCAGGTGAAGCAGATCTACAAGACCCCCCCC | |
| | ATCAAGGACTTCGGCGGCTTCAACTTCAGCCAGATCCTGCCCGACCCC | |
| | AGCAAGCCCAGCAAGAGGAGCCCCATCGAGGACCTGCTGTTCAACAAG | |
| | GTGACCCTGGCCGACGCCGGCTTCATCAAGCAGTACGGCGACTGCCT | |
| | GGGCGACATCGCCGCCAGGGACCTGATCTGCGCCCAGAAGTTCAACG | |
| | GCCTGACCGTGCTGCCCCCCCTGCTGACCGACGAGATGATCGCCCAG | |
| | TACACCAGCGCCCTGCTGGCCGGCACCATCACCAGCGGCTGGACCTT | |
| | CGGCGCCGGCCCCGCCCTGCAGATCCCCTTCCCCATGCAGATGGCCT | |
| | ACAGGTTCAACGGCATCGGCGTGACCCAGAACGTGCTGTACGAGAACC | |
| | AGAAGCTGATCGCCAACCAGTTCAACAGCGCCATCGGCAAGATCCAGG | |
| | ACAGCCTGAGCAGCACCCCCAGCGCCCTGGGCAAGCTGCAGGACGTG | |
| | GTGAACCAGAACGCCCAGGCCCTGAACACCCTGGTGAAGCAGCTGAG | |
| | CAGCAACTTCGGCGCCATCAGCAGCGTGCTGAACGACATCCTGAGCAG | |
| | GCTGGACCCCCCCGAGGCCGAGGTGCAGATCGACAGGCTGATCACCG | |
| | GCAGGCTGCAGAGCCTGCAGACCTACGTGACCCAGCAGCTGATCAGG | |
| | GCCGCCGAGATCAGGGCCAGCGCCAACCTGGCCGCCACCAAGATGAG | |
| | CGAGTGCGTGCTGGGCCAGAGCAAGAGGGTGGACTTCTGCGGCAAGG | |
| | GCTACCACCTGATGAGCTTCCCCCAGAGCGCCCCCCACGGCGTGGTG | |
| | TTCCTGCACGTGACCTACGTGCCCGCCCAGGAGAAGAACTTCACCACC | |
| | GCCCCCGCCATCTGCCACGACGGCAAGGCCCACTTCCCCAGGGAGGG | |
| | CGTGTTCGTGAGCAACGGCACCCACTGGTTCGTGACCCAGAGGAACTT | |
| | CTACGAGCCCCAGATCATCACCACCGACAACACCTTCGTGAGCGGCAA | |
| | CTGCGACGTGGTGATCGGCATCGTGAACAACACCGTGTACGACCCCCT | |
| | GCAGCCCGAGCTGGACAGCTTCAAGGAGGAGCTGGACAAGTACTTCAA | |
| | GAACCACACCAGCCCCGACGTGGACCTGGGCGACATCAGCGGCATCA | |
| | ACGCCAGCGTGGTGAACATCCAGAAGGAGATCGACAGGCTGAACGAG | |
| | GTGGCCAAGAACCTGAACGAGAGCCTGATCGACCTGCAGGAGCTGGG | |
| | CAAGTACGAGCAGTACATCAAGTGGCCCTGGTACATCTGGCTGGGCTT | |
| | CATCGCCGGCCTGATCGCCATCGTGATGGTGACCATCATGCTGTGCTG | |
| | CATGACCAGCTGCTGCAGCTGCCTGAAGGGCTGCTGCAGCTGCGGCA | |
| | GCTGCTGCAAGTTCGACGAGGACGACAGCGAGCCCGTGCTGAAGGGC | |
| | GTGAAGCTGCACTACACCGGAAGCGGAGCCACGAACTTCTCTCTGTTA | |
| | AAGCAAGCAGGAGATGTTGAAGAAAACCCCGGGCCTAGCAGCCAGGC | |
| | CTGGCAGCCCGGCGTGGCCATGCCCAACCTGTACAAGATGCAGAGGA | |
| | TGCTGCTGGAGAAGTGCGACCTGCAGAACTACGGCGACAGCGCCACC | |
| | CTGCCCAAGGGCATCATGATGAACGTGGCCAAGTACACCCAGCTGTGC | |
| | CAGTACCTGAACACCCTGACCCTGGCCGTGCCCTACAACATGAGGGTG | |
| | ATCCACTTCGGCGCCGGCAGCGACAAGGGCGTGGCCCCCGGCACCGC | |
| | CGTGCTGAGGCAGTGGCTGCCCACCGGCACCCTGCTGGTGGACAGCG | |
| | ACCTGAACGACTTCGTGAGCGACGCCGACAGCACCCTGATCGGCGACT | |
| | GCGCCACCGTGCACACCGCCAACAAGTGGGACCTGATCATCAGCGACA | |
| | TGTACGACCCCAAGACCAAGAACGTGACCAAGGAGAACGACAGCAAGG | |
| | AGGGCTTCTTCACCTACATCTGCGGCTTCATCCAGCAGAAGCTGGCCC | |
| | TGGGCGGCAGCGTGGCCATCAAGATCACCGAGCACAGCTGGAACGCC | |
| | GACCTGTACAAGCTGATGGGCCACTTCGCCTGGTGGACCGCCTTCGTG | |
| | ACCAACGTGAACGCCAGCAGCAGCGAGGCCTTCCTGATCGGCTGCAAC | |
| | TACCTGGGCAAGCCCAGGGAGCAGATCGACGGCTACGTGATGCACGC | |
| | CAACTACATCTTCTGGAGGAACACCAACCCCATCCAGCTGAGCAGCTA | |
| | CAGCCTGTTCGACATGAGCAAGTTCCCCCTGAAGCTGAGGGGCACCGC | |
| | CGTGATGAGCCTGAAGGAGGGCCAGATCAACGACATGATCCTGAGCCT | |
| | GCTGAGCAAGGGCAGGCTGATCATCAGGGAGAACAACAGGGTGGTGA | |
| | TCAGCAGCGACGTGCTGGTGAACAACTGATGA | |
| 32 | ATGTTCGTGTTCCTGGTGCTGCTGCCCCTGGTGAGCAGCCAGTGCGTG | 317 |
| Pan-CoV | AACCTGACCACCAGGACCCAGCTGCCCCCCGCCTACACCAACAGCTTC | |
| mRNA Spike- | ACCAGGGGCGTGTACTACCCCGACAAGGTGTTCAGGAGCAGCGTGCT | |
| ORF3a | GCACAGCACCCAGGACCTGTTCCTGCCCTTCTTCAGCAACGTGACCTG | |
| | GTTCCACGCCATCCACGTGAGCGGCACCAACGGCACCAAGAGGTTCGA | |
| | CAACCCCGTGCTGCCCTTCAACGACGGCGTGTACTTCGCCAGCACCGA | |
| | GAAGAGCAACATCATCAGGGGCTGGATCTTCGGCACCACCCTGGACAG | |
| | CAAGACCCAGAGCCTGCTGATCGTGAACAACGCCACCAACGTGGTGAT | |
| | CAAGGTGTGCGAGTTCCAGTTCTGCAACGACCCCTTCCTGGGCGTGTA | |
| | CTACCACAAGAACAACAAGAGCTGGATGGAGAGCGAGTTCAGGGTGTA | |
| | CAGCAGCGCCAACAACTGCACCTTCGAGTACGTGAGCCAGCCCTTCCT | |
| | GATGGACCTGGAGGGCAAGCAGGGCAACTTCAAGAACCTGAGGGAGT | |
| | TCGTGTTCAAGAACATCGACGGCTACTTCAAGATCTACAGCAAGCACAC | |
| | CCCCATCAACCTGGTGAGGGACCTGCCCCAGGGCTTCAGCGCCCTGG | |
| | AGCCCCTGGTGGACCTGCCCATCGGCATCAACATCACCAGGTTCCAGA | |
| | CCCTGCTGGCCCTGCACAGGAGCTACCTGACCCCCGGCGACAGCAGC | |
| | AGCGGCTGGACCGCCGGCGCCGCCGCCTACTACGTGGGCTACCTGCA | |
| | GCCCAGGACCTTCCTGCTGAAGTACAACGAGAACGGCACCATCACCGA | |
| | CGCCGTGGACTGCGCCCTGGACCCCCTGAGCGAGACCAAGTGCACCC | |
| | TGAAGAGCTTCACCGTGGAGAAGGGCATCTACCAGACCAGCAACTTCA | |
| | GGGTGCAGCCCACCGAGAGCATCGTGAGGTTCCCCAACATCACCAACC | |
| | TGTGCCCCTTCGGCGAGGTGTTCAACGCCACCAGGTTCGCCAGCGTGT | |
| | ACGCCTGGAACAGGAAGAGGATCAGCAACTGCGTGGCCGACTACAGC | |
| | GTGCTGTACAACAGCGCCAGCTTCAGCACCTTCAAGTGCTACGGCGTG | |
| | AGCCCCACCAAGCTGAACGACCTGTGCTTCACCAACGTGTACGCCGAC | |
| | AGCTTCGTGATCAGGGGCGACGAGGTGAGGCAGATCGCCCCCGGCCA | |
| | GACCGGCAAGATCGCCGACTACAACTACAAGCTGCCCGACGACTTCAC | |
| | CGGCTGCGTGATCGCCTGGAACAGCAACAACCTGGACAGCAAGGTGG | |
| | GCGGCAACTACAACTACCTGTACAGGCTGTTCAGGAAGAGCAACCTGA | |
| | AGCCCTTCGAGAGGGACATCAGCACCGAGATCTACCAGGCCGGCAGC | |
| | ACCCCCTGCAACGGCGTGGAGGGCTTCAACTGCTACTTCCCCCTGCAG | |
| | AGCTACGGCTTCCAGCCCACCAACGGCGTGGGCTACCAGCCCTACAG | |
| | GGTGGTGGTGCTGAGCTTCGAGCTGCTGCACGCCCCCGCCACCGTGT | |
| | GCGGCCCCAAGAAGAGCACCAACCTGGTGAAGAACAAGTGCGTGAACT | |
| | TCAACTTCAACGGCCTGACCGGCACCGGCGTGCTGACCGAGAGCAACA | |
| | AGAAGTTCCTGCCCTTCCAGCAGTTCGGCAGGGACATCGCCGACACCA | |
| | CCGACGCCGTGAGGGACCCCCAGACCCTGGAGATCCTGGACATCACC | |
| | CCCTGCAGCTTCGGCGGCGTGAGCGTGATCACCCCCGGCACCAACAC | |
| | CAGCAACCAGGTGGCCGTGCTGTACCAGGACGTGAACTGCACCGAGG | |
| | TGCCCGTGGCCATCCACGCCGACCAGCTGACCCCCACCTGGAGGGTG | |
| | TACAGCACCGGCAGCAACGTGTTCCAGACCAGGGCCGGCTGCCTGAT | |
| | CGGCGCCGAGCACGTGAACAACAGCTACGAGTGCGACATCCCCATCG | |
| | GCGCCGGCATCTGCGCCAGCTACCAGACCCAGACCAACAGCCCCAGG | |
| | AGGGCCAGGAGCGTGGCCAGCCAGAGCATCATCGCCTACACCATGAG | |
| | CCTGGGCGCCGAGAACAGCGTGGCCTACAGCAACAACAGCATCGCCA | |
| | TCCCCACCAACTTCACCATCAGCGTGACCACCGAGATCCTGCCCGTGA | |
| | GCATGACCAAGACCAGCGTGGACTGCACCATGTACATCTGCGGCGACA | |
| | GCACCGAGTGCAGCAACCTGCTGCTGCAGTACGGCAGCTTCTGCACCC | |
| | AGCTGAACAGGGCCCTGACCGGCATCGCCGTGGAGCAGGACAAGAAC | |
| | ACCCAGGAGGTGTTCGCCCAGGTGAAGCAGATCTACAAGACCCCCCCC | |
| | ATCAAGGACTTCGGCGGCTTCAACTTCAGCCAGATCCTGCCCGACCCC | |
| | AGCAAGCCCAGCAAGAGGAGCTTCATCGAGGACCTGCTGTTCAACAAG | |
| | GTGACCCTGGCCGACGCCGGCTTCATCAAGCAGTACGGCGACTGCCT | |
| | GGGCGACATCGCCGCCAGGGACCTGATCTGCGCCCAGAAGTTCAACG | |
| | GCCTGACCGTGCTGCCCCCCCTGCTGACCGACGAGATGATCGCCCAG | |
| | TACACCAGCGCCCTGCTGGCCGGCACCATCACCAGCGGCTGGACCTT | |
| | CGGCGCCGGCGCCGCCCTGCAGATCCCCTTCGCCATGCAGATGGCCT | |
| | ACAGGTTCAACGGCATCGGCGTGACCCAGAACGTGCTGTACGAGAACC | |
| | AGAAGCTGATCGCCAACCAGTTCAACAGCGCCATCGGCAAGATCCAGG | |
| | ACAGCCTGAGCAGCACCGCCAGCGCCCTGGGCAAGCTGCAGGACGTG | |
| | GTGAACCAGAACGCCCAGGCCCTGAACACCCTGGTGAAGCAGCTGAG | |
| | CAGCAACTTCGGCGCCATCAGCAGCGTGCTGAACGACATCCTGAGCAG | |
| | GCTGGACCCCCCCGAGGCCGAGGTGCAGATCGACAGGCTGATCACCG | |
| | GCAGGCTGCAGAGCCTGCAGACCTACGTGACCCAGCAGCTGATCAGG | |
| | GCCGCCGAGATCAGGGCCAGCGCCAACCTGGCCGCCACCAAGATGAG | |
| | CGAGTGCGTGCTGGGCCAGAGCAAGAGGGTGGACTTCTGCGGCAAGG | |
| | GCTACCACCTGATGAGCTTCCCCCAGAGCGCCCCCCACGGCGTGGTG | |
| | TTCCTGCACGTGACCTACGTGCCCGCCCAGGAGAAGAACTTCACCACC | |
| | GCCCCCGCCATCTGCCACGACGGCAAGGCCCACTTCCCCAGGGAGGG | |
| | CGTGTTCGTGAGCAACGGCACCCACTGGTTCGTGACCCAGAGGAACTT | |
| | CTACGAGCCCCAGATCATCACCACCGACAACACCTTCGTGAGCGGCAA | |
| | CTGCGACGTGGTGATCGGCATCGTGAACAACACCGTGTACGACCCCCT | |
| | GCAGCCCGAGCTGGACAGCTTCAAGGAGGAGCTGGACAAGTACTTCAA | |
| | GAACCACACCAGCCCCGACGTGGACCTGGGCGACATCAGCGGCATCA | |
| | ACGCCAGCGTGGTGAACATCCAGAAGGAGATCGACAGGCTGAACGAG | |
| | GTGGCCAAGAACCTGAACGAGAGCCTGATCGACCTGCAGGAGCTGGG | |
| | CAAGTACGAGCAGTACATCAAGTGGCCCTGGTACATCTGGCTGGGCTT | |
| | CATCGCCGGCCTGATCGCCATCGTGATGGTGACCATCATGCTGTGCTG | |
| | CATGACCAGCTGCTGCAGCTGCCTGAAGGGCTGCTGCAGCTGCGGCA | |
| | GCTGCTGCAAGTTCGACGAGGACGACAGCGAGCCCGTGCTGAAGGGC | |
| | GTGAAGCTGCACTACACCGGAAGCGGAGCCACGAACTTCTCTCTGTTA | |
| | AAGCAAGCAGGAGATGTTGAAGAAAACCCCGGGCCTATGGACCTGTTC | |
| | ATGAGGATCTTCACCATCGGCACCGTGACCCTGAAGCAGGGCGAGATC | |
| | AAGGACGCCACCCCCAGCGACTTCGTGAGGGCCACCGCCACCATCCC | |
| | CATCCAGGCCAGCCTGCCCTTCGGCTGGCTGATCGTGGGCGTGGCCC | |
| | TGCTGGCCGTGTTCCAGAGCGCCAGCAAGATCATCACCCTGAAGAAGA | |
| | GGTGGCAGCTGGCCCTGAGCAAGGGCGTGCACTTCGTGTGCAACCTG | |
| | CTGCTGCTGTTCGTGACCGTGTACAGCCACCTGCTGCTGGTGGCCGCC | |
| | GGCCTGGAGGCCCCCTTCCTGTACCTGTACGCCCTGGTGTACTTCCTG | |
| | CAGAGCATCAACTTCGTGAGGATCATCATGAGGCTGTGGCTGTGCTGG | |
| | AAGTGCAGGAGCAAGAACCCCCTGCTGTACGACGCCAACTACTTCCTG | |
| | TGCTGGCACACCAACTGCTACGACTACTGCATCCCCTACAACAGCGTG | |
| | ACCAGCAGCATCGTGATCACCAGCGGCGACGGCACCACCAGCCCCAT | |
| | CAGCGAGCACGACTACCAGATCGGCGGCTACACCGAGAAGTGGGAGA | |
| | GCGGCGTGAAGGACTGCGTGGTGCTGCACAGCTACTTCACCAGCGACT | |
| | ACTACCAGCTGTACAGCACCCAGCTGAGCACCGACACCGGCGTGGAG | |
| | CACGTGACCTTCTTCATCTACAACAAGATCGTGGACGAGCCCGAGGAG | |
| | CACGTGCAGATCCACACCATCGACGGCAGCAGCGGCGTGGTGAACCC | |
| | CGTGATGGAGCCCATCTACGACGAGCCCACCACCACCACCAGCGTGC | |
| | CCCTGTGATGA | |
| 33 | ATGTTCGTGTTCCTGGTGCTGCTGCCCCTGGTGAGCAGCCAGTGCGTG | 318 |
| Pan-CoV | AACCTGACCACCAGGACCCAGCTGCCCCCCGCCTACACCAACAGCTTC | |
| mRNA | ACCAGGGGCGTGTACTACCCCGACAAGGTGTTCAGGAGCAGCGTGCT | |
| HexaPro- | GCACAGCACCCAGGACCTGTTCCTGCCCTTCTTCAGCAACGTGACCTG | |
| ORF3a | GTTCCACGCCATCCACGTGAGCGGCACCAACGGCACCAAGAGGTTCGA | |
| | CAACCCCGTGCTGCCCTTCAACGACGGCGTGTACTTCGCCAGCACCGA | |
| | GAAGAGCAACATCATCAGGGGCTGGATCTTCGGCACCACCCTGGACAG | |
| | CAAGACCCAGAGCCTGCTGATCGTGAACAACGCCACCAACGTGGTGAT | |
| | CAAGGTGTGCGAGTTCCAGTTCTGCAACGACCCCTTCCTGGGCGTGTA | |
| | CTACCACAAGAACAACAAGAGCTGGATGGAGAGCGAGTTCAGGGTGTA | |
| | CAGCAGCGCCAACAACTGCACCTTCGAGTACGTGAGCCAGCCCTTCCT | |
| | GATGGACCTGGAGGGCAAGCAGGGCAACTTCAAGAACCTGAGGGAGT | |
| | TCGTGTTCAAGAACATCGACGGCTACTTCAAGATCTACAGCAAGCACAC | |
| | CCCCATCAACCTGGTGAGGGACCTGCCCCAGGGCTTCAGCGCCCTGG | |
| | AGCCCCTGGTGGACCTGCCCATCGGCATCAACATCACCAGGTTCCAGA | |
| | CCCTGCTGGCCCTGCACAGGAGCTACCTGACCCCCGGCGACAGCAGC | |
| | AGCGGCTGGACCGCCGGCGCCGCCGCCTACTACGTGGGCTACCTGCA | |
| | GCCCAGGACCTTCCTGCTGAAGTACAACGAGAACGGCACCATCACCGA | |
| | CGCCGTGGACTGCGCCCTGGACCCCCTGAGCGAGACCAAGTGCACCC | |
| | TGAAGAGCTTCACCGTGGAGAAGGGCATCTACCAGACCAGCAACTTCA | |
| | GGGTGCAGCCCACCGAGAGCATCGTGAGGTTCCCCAACATCACCAACC | |
| | TGTGCCCCTTCGGCGAGGTGTTCAACGCCACCAGGTTCGCCAGCGTGT | |
| | ACGCCTGGAACAGGAAGAGGATCAGCAACTGCGTGGCCGACTACAGC | |
| | GTGCTGTACAACAGCGCCAGCTTCAGCACCTTCAAGTGCTACGGCGTG | |
| | AGCCCCACCAAGCTGAACGACCTGTGCTTCACCAACGTGTACGCCGAC | |
| | AGCTTCGTGATCAGGGGCGACGAGGTGAGGCAGATCGCCCCCGGCCA | |
| | GACCGGCAAGATCGCCGACTACAACTACAAGCTGCCCGACGACTTCAC | |
| | CGGCTGCGTGATCGCCTGGAACAGCAACAACCTGGACAGCAAGGTGG | |
| | GCGGCAACTACAACTACCTGTACAGGCTGTTCAGGAAGAGCAACCTGA | |
| | AGCCCTTCGAGAGGGACATCAGCACCGAGATCTACCAGGCCGGCAGC | |
| | ACCCCCTGCAACGGCGTGGAGGGCTTCAACTGCTACTTCCCCCTGCAG | |
| | AGCTACGGCTTCCAGCCCACCAACGGCGTGGGCTACCAGCCCTACAG | |
| | GGTGGTGGTGCTGAGCTTCGAGCTGCTGCACGCCCCCGCCACCGTGT | |
| | GCGGCCCCAAGAAGAGCACCAACCTGGTGAAGAACAAGTGCGTGAACT | |
| | TCAACTTCAACGGCCTGACCGGCACCGGCGTGCTGACCGAGAGCAACA | |
| | AGAAGTTCCTGCCCTTCCAGCAGTTCGGCAGGGACATCGCCGACACCA | |
| | CCGACGCCGTGAGGGACCCCCAGACCCTGGAGATCCTGGACATCACC | |
| | CCCTGCAGCTTCGGCGGCGTGAGCGTGATCACCCCCGGCACCAACAC | |
| | CAGCAACCAGGTGGCCGTGCTGTACCAGGACGTGAACTGCACCGAGG | |
| | TGCCCGTGGCCATCCACGCCGACCAGCTGACCCCCACCTGGAGGGTG | |
| | TACAGCACCGGCAGCAACGTGTTCCAGACCAGGGCCGGCTGCCTGAT | |
| | CGGCGCCGAGCACGTGAACAACAGCTACGAGTGCGACATCCCCATCG | |
| | GCGCCGGCATCTGCGCCAGCTACCAGACCCAGACCAACAGCCCCGGC | |
| | AGCGCCAGCAGCGTGGCCAGCCAGAGCATCATCGCCTACACCATGAG | |
| | CCTGGGCGCCGAGAACAGCGTGGCCTACAGCAACAACAGCATCGCCA | |
| | TCCCCACCAACTTCACCATCAGCGTGACCACCGAGATCCTGCCCGTGA | |
| | GCATGACCAAGACCAGCGTGGACTGCACCATGTACATCTGCGGCGACA | |
| | GCACCGAGTGCAGCAACCTGCTGCTGCAGTACGGCAGCTTCTGCACCC | |
| | AGCTGAACAGGGCCCTGACCGGCATCGCCGTGGAGCAGGACAAGAAC | |
| | ACCCAGGAGGTGTTCGCCCAGGTGAAGCAGATCTACAAGACCCCCCCC | |
| | ATCAAGGACTTCGGCGGCTTCAACTTCAGCCAGATCCTGCCCGACCCC | |
| | AGCAAGCCCAGCAAGAGGAGCCCCATCGAGGACCTGCTGTTCAACAAG | |
| | GTGACCCTGGCCGACGCCGGCTTCATCAAGCAGTACGGCGACTGCCT | |
| | GGGCGACATCGCCGCCAGGGACCTGATCTGCGCCCAGAAGTTCAACG | |
| | GCCTGACCGTGCTGCCCCCCCTGCTGACCGACGAGATGATCGCCCAG | |
| | TACACCAGCGCCCTGCTGGCCGGCACCATCACCAGCGGCTGGACCTT | |
| | CGGCGCCGGCCCCGCCCTGCAGATCCCCTTCCCCATGCAGATGGCCT | |
| | ACAGGTTCAACGGCATCGGCGTGACCCAGAACGTGCTGTACGAGAACC | |
| | AGAAGCTGATCGCCAACCAGTTCAACAGCGCCATCGGCAAGATCCAGG | |
| | ACAGCCTGAGCAGCACCCCCAGCGCCCTGGGCAAGCTGCAGGACGTG | |
| | GTGAACCAGAACGCCCAGGCCCTGAACACCCTGGTGAAGCAGCTGAG | |
| | CAGCAACTTCGGCGCCATCAGCAGCGTGCTGAACGACATCCTGAGCAG | |
| | GCTGGACCCCCCCGAGGCCGAGGTGCAGATCGACAGGCTGATCACCG | |
| | GCAGGCTGCAGAGCCTGCAGACCTACGTGACCCAGCAGCTGATCAGG | |
| | GCCGCCGAGATCAGGGCCAGCGCCAACCTGGCCGCCACCAAGATGAG | |
| | CGAGTGCGTGCTGGGCCAGAGCAAGAGGGTGGACTTCTGCGGCAAGG | |
| | GCTACCACCTGATGAGCTTCCCCCAGAGCGCCCCCCACGGCGTGGTG | |
| | TTCCTGCACGTGACCTACGTGCCCGCCCAGGAGAAGAACTTCACCACC | |
| | GCCCCCGCCATCTGCCACGACGGCAAGGCCCACTTCCCCAGGGAGGG | |
| | CGTGTTCGTGAGCAACGGCACCCACTGGTTCGTGACCCAGAGGAACTT | |
| | CTACGAGCCCCAGATCATCACCACCGACAACACCTTCGTGAGCGGCAA | |
| | CTGCGACGTGGTGATCGGCATCGTGAACAACACCGTGTACGACCCCCT | |
| | GCAGCCCGAGCTGGACAGCTTCAAGGAGGAGCTGGACAAGTACTTCAA | |
| | GAACCACACCAGCCCCGACGTGGACCTGGGCGACATCAGCGGCATCA | |
| | ACGCCAGCGTGGTGAACATCCAGAAGGAGATCGACAGGCTGAACGAG | |
| | GTGGCCAAGAACCTGAACGAGAGCCTGATCGACCTGCAGGAGCTGGG | |
| | CAAGTACGAGCAGTACATCAAGTGGCCCTGGTACATCTGGCTGGGCTT | |
| | CATCGCCGGCCTGATCGCCATCGTGATGGTGACCATCATGCTGTGCTG | |
| | CATGACCAGCTGCTGCAGCTGCCTGAAGGGCTGCTGCAGCTGCGGCA | |
| | GCTGCTGCAAGTTCGACGAGGACGACAGCGAGCCCGTGCTGAAGGGC | |
| | GTGAAGCTGCACTACACCGGAAGCGGAGCCACGAACTTCTCTCTGTTA | |
| | AAGCAAGCAGGAGATGTTGAAGAAAACCCCGGGCCTATGGACCTGTTC | |
| | ATGAGGATCTTCACCATCGGCACCGTGACCCTGAAGCAGGGCGAGATC | |
| | AAGGACGCCACCCCCAGCGACTTCGTGAGGGCCACCGCCACCATCCC | |
| | CATCCAGGCCAGCCTGCCCTTCGGCTGGCTGATCGTGGGCGTGGCCC | |
| | TGCTGGCCGTGTTCCAGAGCGCCAGCAAGATCATCACCCTGAAGAAGA | |
| | GGTGGCAGCTGGCCCTGAGCAAGGGCGTGCACTTCGTGTGCAACCTG | |
| | CTGCTGCTGTTCGTGACCGTGTACAGCCACCTGCTGCTGGTGGCCGCC | |
| | GGCCTGGAGGCCCCCTTCCTGTACCTGTACGCCCTGGTGTACTTCCTG | |
| | CAGAGCATCAACTTCGTGAGGATCATCATGAGGCTGTGGCTGTGCTGG | |
| | AAGTGCAGGAGCAAGAACCCCCTGCTGTACGACGCCAACTACTTCCTG | |
| | TGCTGGCACACCAACTGCTACGACTACTGCATCCCCTACAACAGCGTG | |
| | ACCAGCAGCATCGTGATCACCAGCGGCGACGGCACCACCAGCCCCAT | |
| | CAGCGAGCACGACTACCAGATCGGCGGCTACACCGAGAAGTGGGAGA | |
| | GCGGCGTGAAGGACTGCGTGGTGCTGCACAGCTACTTCACCAGCGACT | |
| | ACTACCAGCTGTACAGCACCCAGCTGAGCACCGACACCGGCGTGGAG | |
| | CACGTGACCTTCTTCATCTACAACAAGATCGTGGACGAGCCCGAGGAG | |
| | CACGTGCAGATCCACACCATCGACGGCAGCAGCGGCGTGGTGAACCC | |
| | CGTGATGGAGCCCATCTACGACGAGCCCACCACCACCACCAGCGTGC | |
| | CCCTGTGATGA | |
| 34 | CTCGACATTGATTATTGACTAGTTATTAATAGTAATCAATTACGGGGTCA | 319 |
| Pan-CoV | TTAGTTCATAGCCCATATATGGAGTTCCGCGTTACATAACTTACGGTAAA | |
| rAdv5-CAG- | TGGCCCGCCTGGCTGACCGCCCAACGACCCCCGCCCATTGACGTCAAT | |
| HexaPro | AATGACGTATGTTCCCATAGTAACGCCAATAGGGACTTTCCATTGACGT | |
| | CAATGGGTGGAGTATTTACGGTAAACTGCCCACTTGGCAGTACATCAAG | |
| | TGTATCATATGCCAAGTACGCCCCCTATTGACGTCAATGACGGTAAATG | |
| | GCCCGCCTGGCATTATGCCCAGTACATGACCTTATGGGACTTTCCTACT | |
| | TGGCAGTACATCTACGTATTAGTCATCGCTATTACCATGGTCGAGGTGA | |
| | GCCCCACGTTCTGCTTCACTCTCCCCATCTCCCCCCCCTCCCCACCCC | |
| | CAATTTTGTATTTATTTATTTTTTAATTATTTTGTGCAGCGATGGGGGCG | |
| | GGGGGGGGGGGGGGGCGCGCGCCAGGCGGGGCGGGGCGGGGCGA | |
| | GGGGCGGGGCGGGGCGAGGCGGAGAGGTGCGGCGGCAGCCAATCA | |
| | GAGCGGCGCGCTCCGAAAGTTTCCTTTTATGGCGAGGCGGCGGCGGC | |
| | GGCGGCCCTATAAAAAGCGAAGCGCGCGGCGGGCGGGAGTCGCTGC | |
| | GCGCTGCCTTCGCCCCGTGCCCCGCTCCGCCGCCGCCTCGCGCCGCC | |
| | CGCCCCGGCTCTGACTGACCGCGTTACTCCCACAGGTGAGCGGGCGG | |
| | GACGGCCCTTCTCCTCCGGGCTGTAATTAGCGCTTGGTTTAATGACGG | |
| | CTTGTTTCTTTTCTGTGGCTGCGTGAAAGCCTTGAGGGGCTCCGGGAG | |
| | GGCCCTTTGTGCGGGGGGAGCGGCTCGGGGGGTGCGTGCGTGTGTG | |
| | TGTGCGTGGGGAGCGCCGCGTGCGGCTCCGCGCTGCCCGGCGGCTG | |
| | TGAGCGCTGCGGGCGCGGCGCGGGGCTTTGTGCGCTCCGCAGTGTGC | |
| | GCGAGGGGAGCGCGGCCGGGGGCGGTGCCCCGCGGTGCGGGGGGG | |
| | GCTGCGAGGGGAACAAAGGCTGCGTGCGGGGTGTGTGCGTGGGGGG | |
| | GTGAGCAGGGGGTGTGGGCGCGTCGGTCGGGCTGCAACCCCCCCTG | |
| | CACCCCCCTCCCCGAGTTGCTGAGCACGGCCCGGCTTCGGGTGCGGG | |
| | GCTCCGTACGGGGCGTGGCGCGGGGCTCGCCGTGCCGGGCGGGGGG | |
| | TGGCGGCAGGTGGGGGTGCCGGGCGGGGCGGGGCCGCCTCGGGCC | |
| | GGGGAGGGCTCGGGGGAGGGGCGCGGCGGCCCCCGGAGCGCCGGC | |
| | GGCTGTCGAGGCGCGGCGAGCCGCAGCCATTGCCTTTTATGGTAATCG | |
| | TGCGAGAGGGCGCAGGGACTTCCTTTGTCCCAAATCTGTGCGGAGCCG | |
| | AAATCTGGGAGGCGCCGCCGCACCCCCTCTAGCGGGCGCGGGGCGAA | |
| | GCGGTGCGGCGCCGGCAGGAAGGAAATGGGCGGGGAGGGCCTTCGT | |
| | GCGTCGCCGCGCCGCCGTCCCCTTCTCCCTCTCCAGCCTCGGGGCTG | |
| | TCCGCGGGGGGACGGCTGCCTTCGGGGGGGACGGGGCAGGGCGGG | |
| | GTTCGGCTTCTGGCGTGTGACCGGCGGCTCTAGAGCCTCTGCTAACCA | |
| | TGTTCATGCCTTCTTCTTTTTCCTACAGCTCCTGGGCAACGTGCTGGTT | |
| | ATTGTGCTGTCTCATCATTTTGGCAAAGAATTGGAGAATAAACTAGTATT | |
| | CTTCTGGTCCCCACAGACTCAGAGAGAACCCGCCACCATGTTCGTGTT | |
| | CCTGGTGCTGCTGCCCCTGGTGAGCAGCCAGTGCGTGAACCTGACCA | |
| | CCAGGACCCAGCTGCCCCCCGCCTACACCAACAGCTTCACCAGGGGC | |
| | GTGTACTACCCCGACAAGGTGTTCAGGAGCAGCGTGCTGCACAGCACC | |
| | CAGGACCTGTTCCTGCCCTTCTTCAGCAACGTGACCTGGTTCCACGCC | |
| | ATCCACGTGAGCGGCACCAACGGCACCAAGAGGTTCGACAACCCCGT | |
| | GCTGCCCTTCAACGACGGCGTGTACTTCGCCAGCACCGAGAAGAGCAA | |
| | CATCATCAGGGGCTGGATCTTCGGCACCACCCTGGACAGCAAGACCCA | |
| | GAGCCTGCTGATCGTGAACAACGCCACCAACGTGGTGATCAAGGTGTG | |
| | CGAGTTCCAGTTCTGCAACGACCCCTTCCTGGGCGTGTACTACCACAA | |
| | GAACAACAAGAGCTGGATGGAGAGCGAGTTCAGGGTGTACAGCAGCG | |
| | CCAACAACTGCACCTTCGAGTACGTGAGCCAGCCCTTCCTGATGGACC | |
| | TGGAGGGCAAGCAGGGCAACTTCAAGAACCTGAGGGAGTTCGTGTTCA | |
| | AGAACATCGACGGCTACTTCAAGATCTACAGCAAGCACACCCCCATCAA | |
| | CCTGGTGAGGGACCTGCCCCAGGGCTTCAGCGCCCTGGAGCCCCTGG | |
| | TGGACCTGCCCATCGGCATCAACATCACCAGGTTCCAGACCCTGCTGG | |
| | CCCTGCACAGGAGCTACCTGACCCCCGGCGACAGCAGCAGCGGCTGG | |
| | ACCGCCGGCGCCGCCGCCTACTACGTGGGCTACCTGCAGCCCAGGAC | |
| | CTTCCTGCTGAAGTACAACGAGAACGGCACCATCACCGACGCCGTGGA | |
| | CTGCGCCCTGGACCCCCTGAGCGAGACCAAGTGCACCCTGAAGAGCTT | |
| | CACCGTGGAGAAGGGCATCTACCAGACCAGCAACTTCAGGGTGCAGCC | |
| | CACCGAGAGCATCGTGAGGTTCCCCAACATCACCAACCTGTGCCCCTT | |
| | CGGCGAGGTGTTCAACGCCACCAGGTTCGCCAGCGTGTACGCCTGGA | |
| | ACAGGAAGAGGATCAGCAACTGCGTGGCCGACTACAGCGTGCTGTACA | |
| | ACAGCGCCAGCTTCAGCACCTTCAAGTGCTACGGCGTGAGCCCCACCA | |
| | AGCTGAACGACCTGTGCTTCACCAACGTGTACGCCGACAGCTTCGTGA | |
| | TCAGGGGCGACGAGGTGAGGCAGATCGCCCCCGGCCAGACCGGCAA | |
| | GATCGCCGACTACAACTACAAGCTGCCCGACGACTTCACCGGCTGCGT | |
| | GATCGCCTGGAACAGCAACAACCTGGACAGCAAGGTGGGCGGCAACT | |
| | ACAACTACCTGTACAGGCTGTTCAGGAAGAGCAACCTGAAGCCCTTCG | |
| | AGAGGGACATCAGCACCGAGATCTACCAGGCCGGCAGCACCCCCTGC | |
| | AACGGCGTGGAGGGCTTCAACTGCTACTTCCCCCTGCAGAGCTACGGC | |
| | TTCCAGCCCACCAACGGCGTGGGCTACCAGCCCTACAGGGTGGTGGT | |
| | GCTGAGCTTCGAGCTGCTGCACGCCCCCGCCACCGTGTGCGGCCCCA | |
| | AGAAGAGCACCAACCTGGTGAAGAACAAGTGCGTGAACTTCAACTTCAA | |
| | CGGCCTGACCGGCACCGGCGTGCTGACCGAGAGCAACAAGAAGTTCC | |
| | TGCCCTTCCAGCAGTTCGGCAGGGACATCGCCGACACCACCGACGCC | |
| | GTGAGGGACCCCCAGACCCTGGAGATCCTGGACATCACCCCCTGCAG | |
| | CTTCGGCGGCGTGAGCGTGATCACCCCCGGCACCAACACCAGCAACC | |
| | AGGTGGCCGTGCTGTACCAGGACGTGAACTGCACCGAGGTGCCCGTG | |
| | GCCATCCACGCCGACCAGCTGACCCCCACCTGGAGGGTGTACAGCAC | |
| | CGGCAGCAACGTGTTCCAGACCAGGGCCGGCTGCCTGATCGGCGCCG | |
| | AGCACGTGAACAACAGCTACGAGTGCGACATCCCCATCGGCGCCGGC | |
| | ATCTGCGCCAGCTACCAGACCCAGACCAACAGCCCCGGCAGCGCCAG | |
| | CAGCGTGGCCAGCCAGAGCATCATCGCCTACACCATGAGCCTGGGCG | |
| | CCGAGAACAGCGTGGCCTACAGCAACAACAGCATCGCCATCCCCACCA | |
| | ACTTCACCATCAGCGTGACCACCGAGATCCTGCCCGTGAGCATGACCA | |
| | AGACCAGCGTGGACTGCACCATGTACATCTGCGGCGACAGCACCGAGT | |
| | GCAGCAACCTGCTGCTGCAGTACGGCAGCTTCTGCACCCAGCTGAACA | |
| | GGGCCCTGACCGGCATCGCCGTGGAGCAGGACAAGAACACCCAGGAG | |
| | GTGTTCGCCCAGGTGAAGCAGATCTACAAGACCCCCCCCATCAAGGAC | |
| | TTCGGCGGCTTCAACTTCAGCCAGATCCTGCCCGACCCCAGCAAGCCC | |
| | AGCAAGAGGAGCCCCATCGAGGACCTGCTGTTCAACAAGGTGACCCTG | |
| | GCCGACGCCGGCTTCATCAAGCAGTACGGCGACTGCCTGGGCGACAT | |
| | CGCCGCCAGGGACCTGATCTGCGCCCAGAAGTTCAACGGCCTGACCG | |
| | TGCTGCCCCCCCTGCTGACCGACGAGATGATCGCCCAGTACACCAGCG | |
| | CCCTGCTGGCCGGCACCATCACCAGCGGCTGGACCTTCGGCGCCGGC | |
| | CCCGCCCTGCAGATCCCCTTCCCCATGCAGATGGCCTACAGGTTCAAC | |
| | GGCATCGGCGTGACCCAGAACGTGCTGTACGAGAACCAGAAGCTGATC | |
| | GCCAACCAGTTCAACAGCGCCATCGGCAAGATCCAGGACAGCCTGAGC | |
| | AGCACCCCCAGCGCCCTGGGCAAGCTGCAGGACGTGGTGAACCAGAA | |
| | CGCCCAGGCCCTGAACACCCTGGTGAAGCAGCTGAGCAGCAACTTCG | |
| | GCGCCATCAGCAGCGTGCTGAACGACATCCTGAGCAGGCTGGACCCC | |
| | CCCGAGGCCGAGGTGCAGATCGACAGGCTGATCACCGGCAGGCTGCA | |
| | GAGCCTGCAGACCTACGTGACCCAGCAGCTGATCAGGGCCGCCGAGA | |
| | TCAGGGCCAGCGCCAACCTGGCCGCCACCAAGATGAGCGAGTGCGTG | |
| | CTGGGCCAGAGCAAGAGGGTGGACTTCTGCGGCAAGGGCTACCACCT | |
| | GATGAGCTTCCCCCAGAGCGCCCCCCACGGCGTGGTGTTCCTGCACG | |
| | TGACCTACGTGCCCGCCCAGGAGAAGAACTTCACCACCGCCCCCGCCA | |
| | TCTGCCACGACGGCAAGGCCCACTTCCCCAGGGAGGGCGTGTTCGTG | |
| | AGCAACGGCACCCACTGGTTCGTGACCCAGAGGAACTTCTACGAGCCC | |
| | CAGATCATCACCACCGACAACACCTTCGTGAGCGGCAACTGCGACGTG | |
| | GTGATCGGCATCGTGAACAACACCGTGTACGACCCCCTGCAGCCCGAG | |
| | CTGGACAGCTTCAAGGAGGAGCTGGACAAGTACTTCAAGAACCACACC | |
| | AGCCCCGACGTGGACCTGGGCGACATCAGCGGCATCAACGCCAGCGT | |
| | GGTGAACATCCAGAAGGAGATCGACAGGCTGAACGAGGTGGCCAAGA | |
| | ACCTGAACGAGAGCCTGATCGACCTGCAGGAGCTGGGCAAGTACGAG | |
| | CAGTACATCAAGTGGCCCTGGTACATCTGGCTGGGCTTCATCGCCGGC | |
| | CTGATCGCCATCGTGATGGTGACCATCATGCTGTGCTGCATGACCAGC | |
| | TGCTGCAGCTGCCTGAAGGGCTGCTGCAGCTGCGGCAGCTGCTGCAA | |
| | GTTCGACGAGGACGACAGCGAGCCCGTGCTGAAGGGCGTGAAGCTGC | |
| | ACTACACCTGATGACTCGAGCTGGTACTGCATGCACGCAATGCTAGCT | |
| | GCCCCTTTCCCGTCCTGGGTACCCCGAGTCTCCCCCGACCTCGGGTCC | |
| | CAGGTATGCTCCCACCTCCACCTGCCCCACTCACCACCTCTGCTAGTTC | |
| | CAGACACCTCCCAAGCACGCAGCAATGCAGCTCAAAACGCTTAGCCTA | |
| | GCCACACCCCCACGGGAAACAGCAGTGATTAACCTTTAGCAATAAACG | |
| | AAAGTTTAACTAAGCTATACTAACCCCAGGGTTGGTCAATTTCGTGCCA | |
| | GCCACACCCTGGAGCTAGCAAAAAAAA | |
| 35 | CTCGACATTGATTATTGACTAGTTATTAATAGTAATCAATTACGGGGTCA | 320 |
| Pan-CoV | TTAGTTCATAGCCCATATATGGAGTTCCGCGTTACATAACTTACGGTAAA | |
| rAdv5-CAG- | TGGCCCGCCTGGCTGACCGCCCAACGACCCCCGCCCATTGACGTCAAT | |
| Two Proline | AATGACGTATGTTCCCATAGTAACGCCAATAGGGACTTTCCATTGACGT | |
| substitution | CAATGGGTGGAGTATTTACGGTAAACTGCCCACTTGGCAGTACATCAAG | |
| | TGTATCATATGCCAAGTACGCCCCCTATTGACGTCAATGACGGTAAATG | |
| | GCCCGCCTGGCATTATGCCCAGTACATGACCTTATGGGACTTTCCTACT | |
| | TGGCAGTACATCTACGTATTAGTCATCGCTATTACCATGGTCGAGGTGA | |
| | GCCCCACGTTCTGCTTCACTCTCCCCATCTCCCCCCCCTCCCCACCCC | |
| | CAATTTTGTATTTATTTATTTTTTAATTATTTTGTGCAGCGATGGGGGCG | |
| | GGGGGGGGGGGGGGGCGCGCGCCAGGCGGGGCGGGGCGGGGCGA | |
| | GGGGCGGGGCGGGGCGAGGCGGAGAGGTGCGGCGGCAGCCAATCA | |
| | GAGCGGCGCGCTCCGAAAGTTTCCTTTTATGGCGAGGCGGCGGCGGC | |
| | GGCGGCCCTATAAAAAGCGAAGCGCGCGGCGGGCGGGAGTCGCTGC | |
| | GCGCTGCCTTCGCCCCGTGCCCCGCTCCGCCGCCGCCTCGCGCCGCC | |
| | CGCCCCGGCTCTGACTGACCGCGTTACTCCCACAGGTGAGCGGGCGG | |
| | GACGGCCCTTCTCCTCCGGGCTGTAATTAGCGCTTGGTTTAATGACGG | |
| | CTTGTTTCTTTTCTGTGGCTGCGTGAAAGCCTTGAGGGGCTCCGGGAG | |
| | GGCCCTTTGTGCGGGGGGAGCGGCTCGGGGGGTGCGTGCGTGTGTG | |
| | TGTGCGTGGGGAGCGCCGCGTGCGGCTCCGCGCTGCCCGGCGGCTG | |
| | TGAGCGCTGCGGGCGCGGCGCGGGGCTTTGTGCGCTCCGCAGTGTGC | |
| | GCGAGGGGAGCGCGGCCGGGGGCGGTGCCCCGCGGTGCGGGGGGG | |
| | GCTGCGAGGGGAACAAAGGCTGCGTGCGGGGTGTGTGCGTGGGGGG | |
| | GTGAGCAGGGGGTGTGGGCGCGTCGGTCGGGCTGCAACCCCCCCTG | |
| | CACCCCCCTCCCCGAGTTGCTGAGCACGGCCCGGCTTCGGGTGCGGG | |
| | GCTCCGTACGGGGCGTGGCGCGGGGCTCGCCGTGCCGGGCGGGGGG | |
| | TGGCGGCAGGTGGGGGTGCCGGGCGGGGCGGGGCCGCCTCGGGCC | |
| | GGGGAGGGCTCGGGGGAGGGGCGCGGCGGCCCCCGGAGCGCCGGC | |
| | GGCTGTCGAGGCGCGGCGAGCCGCAGCCATTGCCTTTTATGGTAATCG | |
| | TGCGAGAGGGCGCAGGGACTTCCTTTGTCCCAAATCTGTGCGGAGCCG | |
| | AAATCTGGGAGGCGCCGCCGCACCCCCTCTAGCGGGCGCGGGGCGAA | |
| | GCGGTGCGGCGCCGGCAGGAAGGAAATGGGCGGGGAGGGCCTTCGT | |
| | GCGTCGCCGCGCCGCCGTCCCCTTCTCCCTCTCCAGCCTCGGGGCTG | |
| | TCCGCGGGGGGACGGCTGCCTTCGGGGGGGACGGGGCAGGGCGGG | |
| | GTTCGGCTTCTGGCGTGTGACCGGCGGCTCTAGAGCCTCTGCTAACCA | |
| | TGTTCATGCCTTCTTCTTTTTCCTACAGCTCCTGGGCAACGTGCTGGTT | |
| | ATTGTGCTGTCTCATCATTTTGGCAAAGAATTGGAGAATAAACTAGTATT | |
| | CTTCTGGTCCCCACAGACTCAGAGAGAACCCGCCACCATGTTCGTGTT | |
| | CCTGGTGCTGCTGCCCCTGGTGAGCAGCCAGTGCGTGAACCTGACCA | |
| | CCAGGACCCAGCTGCCCCCCGCCTACACCAACAGCTTCACCAGGGGC | |
| | GTGTACTACCCCGACAAGGTGTTCAGGAGCAGCGTGCTGCACAGCACC | |
| | CAGGACCTGTTCCTGCCCTTCTTCAGCAACGTGACCTGGTTCCACGCC | |
| | ATCCACGTGAGCGGCACCAACGGCACCAAGAGGTTCGACAACCCCGT | |
| | GCTGCCCTTCAACGACGGCGTGTACTTCGCCAGCACCGAGAAGAGCAA | |
| | CATCATCAGGGGCTGGATCTTCGGCACCACCCTGGACAGCAAGACCCA | |
| | GAGCCTGCTGATCGTGAACAACGCCACCAACGTGGTGATCAAGGTGTG | |
| | CGAGTTCCAGTTCTGCAACGACCCCTTCCTGGGCGTGTACTACCACAA | |
| | GAACAACAAGAGCTGGATGGAGAGCGAGTTCAGGGTGTACAGCAGCG | |
| | CCAACAACTGCACCTTCGAGTACGTGAGCCAGCCCTTCCTGATGGACC | |
| | TGGAGGGCAAGCAGGGCAACTTCAAGAACCTGAGGGAGTTCGTGTTCA | |
| | AGAACATCGACGGCTACTTCAAGATCTACAGCAAGCACACCCCCATCAA | |
| | CCTGGTGAGGGACCTGCCCCAGGGCTTCAGCGCCCTGGAGCCCCTGG | |
| | TGGACCTGCCCATCGGCATCAACATCACCAGGTTCCAGACCCTGCTGG | |
| | CCCTGCACAGGAGCTACCTGACCCCCGGCGACAGCAGCAGCGGCTGG | |
| | ACCGCCGGCGCCGCCGCCTACTACGTGGGCTACCTGCAGCCCAGGAC | |
| | CTTCCTGCTGAAGTACAACGAGAACGGCACCATCACCGACGCCGTGGA | |
| | CTGCGCCCTGGACCCCCTGAGCGAGACCAAGTGCACCCTGAAGAGCTT | |
| | CACCGTGGAGAAGGGCATCTACCAGACCAGCAACTTCAGGGTGCAGCC | |
| | CACCGAGAGCATCGTGAGGTTCCCCAACATCACCAACCTGTGCCCCTT | |
| | CGGCGAGGTGTTCAACGCCACCAGGTTCGCCAGCGTGTACGCCTGGA | |
| | ACAGGAAGAGGATCAGCAACTGCGTGGCCGACTACAGCGTGCTGTACA | |
| | ACAGCGCCAGCTTCAGCACCTTCAAGTGCTACGGCGTGAGCCCCACCA | |
| | AGCTGAACGACCTGTGCTTCACCAACGTGTACGCCGACAGCTTCGTGA | |
| | TCAGGGGCGACGAGGTGAGGCAGATCGCCCCCGGCCAGACCGGCAA | |
| | GATCGCCGACTACAACTACAAGCTGCCCGACGACTTCACCGGCTGCGT | |
| | GATCGCCTGGAACAGCAACAACCTGGACAGCAAGGTGGGCGGCAACT | |
| | ACAACTACCTGTACAGGCTGTTCAGGAAGAGCAACCTGAAGCCCTTCG | |
| | AGAGGGACATCAGCACCGAGATCTACCAGGCCGGCAGCACCCCCTGC | |
| | AACGGCGTGGAGGGCTTCAACTGCTACTTCCCCCTGCAGAGCTACGGC | |
| | TTCCAGCCCACCAACGGCGTGGGCTACCAGCCCTACAGGGTGGTGGT | |
| | GCTGAGCTTCGAGCTGCTGCACGCCCCCGCCACCGTGTGCGGCCCCA | |
| | AGAAGAGCACCAACCTGGTGAAGAACAAGTGCGTGAACTTCAACTTCAA | |
| | CGGCCTGACCGGCACCGGCGTGCTGACCGAGAGCAACAAGAAGTTCC | |
| | TGCCCTTCCAGCAGTTCGGCAGGGACATCGCCGACACCACCGACGCC | |
| | GTGAGGGACCCCCAGACCCTGGAGATCCTGGACATCACCCCCTGCAG | |
| | CTTCGGCGGCGTGAGCGTGATCACCCCCGGCACCAACACCAGCAACC | |
| | AGGTGGCCGTGCTGTACCAGGACGTGAACTGCACCGAGGTGCCCGTG | |
| | GCCATCCACGCCGACCAGCTGACCCCCACCTGGAGGGTGTACAGCAC | |
| | CGGCAGCAACGTGTTCCAGACCAGGGCCGGCTGCCTGATCGGCGCCG | |
| | AGCACGTGAACAACAGCTACGAGTGCGACATCCCCATCGGCGCCGGC | |
| | ATCTGCGCCAGCTACCAGACCCAGACCAACAGCCCCAGGAGGGCCAG | |
| | GAGCGTGGCCAGCCAGAGCATCATCGCCTACACCATGAGCCTGGGCG | |
| | CCGAGAACAGCGTGGCCTACAGCAACAACAGCATCGCCATCCCCACCA | |
| | ACTTCACCATCAGCGTGACCACCGAGATCCTGCCCGTGAGCATGACCA | |
| | AGACCAGCGTGGACTGCACCATGTACATCTGCGGCGACAGCACCGAGT | |
| | GCAGCAACCTGCTGCTGCAGTACGGCAGCTTCTGCACCCAGCTGAACA | |
| | GGGCCCTGACCGGCATCGCCGTGGAGCAGGACAAGAACACCCAGGAG | |
| | GTGTTCGCCCAGGTGAAGCAGATCTACAAGACCCCCCCCATCAAGGAC | |
| | TTCGGCGGCTTCAACTTCAGCCAGATCCTGCCCGACCCCAGCAAGCCC | |
| | AGCAAGAGGAGCTTCATCGAGGACCTGCTGTTCAACAAGGTGACCCTG | |
| | GCCGACGCCGGCTTCATCAAGCAGTACGGCGACTGCCTGGGCGACAT | |
| | CGCCGCCAGGGACCTGATCTGCGCCCAGAAGTTCAACGGCCTGACCG | |
| | TGCTGCCCCCCCTGCTGACCGACGAGATGATCGCCCAGTACACCAGCG | |
| | CCCTGCTGGCCGGCACCATCACCAGCGGCTGGACCTTCGGCGCCGGC | |
| | GCCGCCCTGCAGATCCCCTTCGCCATGCAGATGGCCTACAGGTTCAAC | |
| | GGCATCGGCGTGACCCAGAACGTGCTGTACGAGAACCAGAAGCTGATC | |
| | GCCAACCAGTTCAACAGCGCCATCGGCAAGATCCAGGACAGCCTGAGC | |
| | AGCACCGCCAGCGCCCTGGGCAAGCTGCAGGACGTGGTGAACCAGAA | |
| | CGCCCAGGCCCTGAACACCCTGGTGAAGCAGCTGAGCAGCAACTTCG | |
| | GCGCCATCAGCAGCGTGCTGAACGACATCCTGAGCAGGCTGGACCCC | |
| | CCCGAGGCCGAGGTGCAGATCGACAGGCTGATCACCGGCAGGCTGCA | |
| | GAGCCTGCAGACCTACGTGACCCAGCAGCTGATCAGGGCCGCCGAGA | |
| | TCAGGGCCAGCGCCAACCTGGCCGCCACCAAGATGAGCGAGTGCGTG | |
| | CTGGGCCAGAGCAAGAGGGTGGACTTCTGCGGCAAGGGCTACCACCT | |
| | GATGAGCTTCCCCCAGAGCGCCCCCCACGGCGTGGTGTTCCTGCACG | |
| | TGACCTACGTGCCCGCCCAGGAGAAGAACTTCACCACCGCCCCCGCCA | |
| | TCTGCCACGACGGCAAGGCCCACTTCCCCAGGGAGGGCGTGTTCGTG | |
| | AGCAACGGCACCCACTGGTTCGTGACCCAGAGGAACTTCTACGAGCCC | |
| | CAGATCATCACCACCGACAACACCTTCGTGAGCGGCAACTGCGACGTG | |
| | GTGATCGGCATCGTGAACAACACCGTGTACGACCCCCTGCAGCCCGAG | |
| | CTGGACAGCTTCAAGGAGGAGCTGGACAAGTACTTCAAGAACCACACC | |
| | AGCCCCGACGTGGACCTGGGCGACATCAGCGGCATCAACGCCAGCGT | |
| | GGTGAACATCCAGAAGGAGATCGACAGGCTGAACGAGGTGGCCAAGA | |
| | ACCTGAACGAGAGCCTGATCGACCTGCAGGAGCTGGGCAAGTACGAG | |
| | CAGTACATCAAGTGGCCCTGGTACATCTGGCTGGGCTTCATCGCCGGC | |
| | CTGATCGCCATCGTGATGGTGACCATCATGCTGTGCTGCATGACCAGC | |
| | TGCTGCAGCTGCCTGAAGGGCTGCTGCAGCTGCGGCAGCTGCTGCAA | |
| | GTTCGACGAGGACGACAGCGAGCCCGTGCTGAAGGGCGTGAAGCTGC | |
| | ACTACACCTGATGACTCGAGCTGGTACTGCATGCACGCAATGCTAGCT | |
| | GCCCCTTTCCCGTCCTGGGTACCCCGAGTCTCCCCCGACCTCGGGTCC | |
| | CAGGTATGCTCCCACCTCCACCTGCCCCACTCACCACCTCTGCTAGTTC | |
| | CAGACACCTCCCAAGCACGCAGCAATGCAGCTCAAAACGCTTAGCCTA | |
| | GCCACACCCCCACGGGAAACAGCAGTGATTAACCTTTAGCAATAAACG | |
| | AAAGTTTAACTAAGCTATACTAACCCCAGGGTTGGTCAATTTCGTGCCA | |
| | GCCACACCCTGGAGCTAGCAAAAAAAA | |
| 36 | TAGTTATTAATAGTAATCAATTACGGGGTCATTAGTTCATAGCCCATATA | 321 |
| Pan-CoV | TGGAGTTCCGCGTTACATAACTTACGGTAAATGGCCCGCCTGGCTGAC | |
| rAdv5-CMV- | CGCCCAACGACCCCCGCCCATTGACGTCAATAATGACGTATGTTCCCAT | |
| HexaPro | AGTAACGCCAATAGGGACTTTCCATTGACGTCAATGGGTGGAGTATTTA | |
| | CGGTAAACTGCCCACTTGGCAGTACATCAAGTGTATCATATGCCAAGTA | |
| | CGCCCCCTATTGACGTCAATGACGGTAAATGGCCCGCCTGGCATTATG | |
| | CCCAGTACATGACCTTATGGGACTTTCCTACTTGGCAGTACATCTACGT | |
| | ATTAGTCATCGCTATTACCATGGTGATGCGGTTTTGGCAGTACATCAAT | |
| | GGGCGTGGATAGCGGTTTGACTCACGGGGATTTCCAAGTCTCCACCCC | |
| | ATTGACGTCAATGGGAGTTTGTTTTGGCACCAAAATCAACGGGACTTTC | |
| | CAAAATGTCGTAACAACTCCGCCCCATTGACGCAAATGGGCGGTAGGC | |
| | GTGTACGGTGGGAGGTCTATATAAGCAGAGCTGGTTTAGTGAACCGTC | |
| | AGATCGAGAATAAACTAGTATTCTTCTGGTCCCCACAGACTCAGAGAGA | |
| | ACCCGCCACCATGTTCGTGTTCCTGGTGCTGCTGCCCCTGGTGAGCAG | |
| | CCAGTGCGTGAACCTGACCACCAGGACCCAGCTGCCCCCCGCCTACA | |
| | CCAACAGCTTCACCAGGGGCGTGTACTACCCCGACAAGGTGTTCAGGA | |
| | GCAGCGTGCTGCACAGCACCCAGGACCTGTTCCTGCCCTTCTTCAGCA | |
| | ACGTGACCTGGTTCCACGCCATCCACGTGAGCGGCACCAACGGCACCA | |
| | AGAGGTTCGACAACCCCGTGCTGCCCTTCAACGACGGCGTGTACTTCG | |
| | CCAGCACCGAGAAGAGCAACATCATCAGGGGCTGGATCTTCGGCACCA | |
| | CCCTGGACAGCAAGACCCAGAGCCTGCTGATCGTGAACAACGCCACCA | |
| | ACGTGGTGATCAAGGTGTGCGAGTTCCAGTTCTGCAACGACCCCTTCC | |
| | TGGGCGTGTACTACCACAAGAACAACAAGAGCTGGATGGAGAGCGAGT | |
| | TCAGGGTGTACAGCAGCGCCAACAACTGCACCTTCGAGTACGTGAGCC | |
| | AGCCCTTCCTGATGGACCTGGAGGGCAAGCAGGGCAACTTCAAGAACC | |
| | TGAGGGAGTTCGTGTTCAAGAACATCGACGGCTACTTCAAGATCTACAG | |
| | CAAGCACACCCCCATCAACCTGGTGAGGGACCTGCCCCAGGGCTTCAG | |
| | CGCCCTGGAGCCCCTGGTGGACCTGCCCATCGGCATCAACATCACCAG | |
| | GTTCCAGACCCTGCTGGCCCTGCACAGGAGCTACCTGACCCCCGGCG | |
| | ACAGCAGCAGCGGCTGGACCGCCGGCGCCGCCGCCTACTACGTGGGC | |
| | TACCTGCAGCCCAGGACCTTCCTGCTGAAGTACAACGAGAACGGCACC | |
| | ATCACCGACGCCGTGGACTGCGCCCTGGACCCCCTGAGCGAGACCAA | |
| | GTGCACCCTGAAGAGCTTCACCGTGGAGAAGGGCATCTACCAGACCAG | |
| | CAACTTCAGGGTGCAGCCCACCGAGAGCATCGTGAGGTTCCCCAACAT | |
| | CACCAACCTGTGCCCCTTCGGCGAGGTGTTCAACGCCACCAGGTTCGC | |
| | CAGCGTGTACGCCTGGAACAGGAAGAGGATCAGCAACTGCGTGGCCG | |
| | ACTACAGCGTGCTGTACAACAGCGCCAGCTTCAGCACCTTCAAGTGCT | |
| | ACGGCGTGAGCCCCACCAAGCTGAACGACCTGTGCTTCACCAACGTGT | |
| | ACGCCGACAGCTTCGTGATCAGGGGCGACGAGGTGAGGCAGATCGCC | |
| | CCCGGCCAGACCGGCAAGATCGCCGACTACAACTACAAGCTGCCCGA | |
| | CGACTTCACCGGCTGCGTGATCGCCTGGAACAGCAACAACCTGGACAG | |
| | CAAGGTGGGCGGCAACTACAACTACCTGTACAGGCTGTTCAGGAAGAG | |
| | CAACCTGAAGCCCTTCGAGAGGGACATCAGCACCGAGATCTACCAGGC | |
| | CGGCAGCACCCCCTGCAACGGCGTGGAGGGCTTCAACTGCTACTTCCC | |
| | CCTGCAGAGCTACGGCTTCCAGCCCACCAACGGCGTGGGCTACCAGC | |
| | CCTACAGGGTGGTGGTGCTGAGCTTCGAGCTGCTGCACGCCCCCGCC | |
| | ACCGTGTGCGGCCCCAAGAAGAGCACCAACCTGGTGAAGAACAAGTGC | |
| | GTGAACTTCAACTTCAACGGCCTGACCGGCACCGGCGTGCTGACCGAG | |
| | AGCAACAAGAAGTTCCTGCCCTTCCAGCAGTTCGGCAGGGACATCGCC | |
| | GACACCACCGACGCCGTGAGGGACCCCCAGACCCTGGAGATCCTGGA | |
| | CATCACCCCCTGCAGCTTCGGCGGCGTGAGCGTGATCACCCCCGGCA | |
| | CCAACACCAGCAACCAGGTGGCCGTGCTGTACCAGGACGTGAACTGCA | |
| | CCGAGGTGCCCGTGGCCATCCACGCCGACCAGCTGACCCCCACCTGG | |
| | AGGGTGTACAGCACCGGCAGCAACGTGTTCCAGACCAGGGCCGGCTG | |
| | CCTGATCGGCGCCGAGCACGTGAACAACAGCTACGAGTGCGACATCCC | |
| | CATCGGCGCCGGCATCTGCGCCAGCTACCAGACCCAGACCAACAGCC | |
| | CCGGCAGCGCCAGCAGCGTGGCCAGCCAGAGCATCATCGCCTACACC | |
| | ATGAGCCTGGGCGCCGAGAACAGCGTGGCCTACAGCAACAACAGCAT | |
| | CGCCATCCCCACCAACTTCACCATCAGCGTGACCACCGAGATCCTGCC | |
| | CGTGAGCATGACCAAGACCAGCGTGGACTGCACCATGTACATCTGCGG | |
| | CGACAGCACCGAGTGCAGCAACCTGCTGCTGCAGTACGGCAGCTTCTG | |
| | CACCCAGCTGAACAGGGCCCTGACCGGCATCGCCGTGGAGCAGGACA | |
| | AGAACACCCAGGAGGTGTTCGCCCAGGTGAAGCAGATCTACAAGACCC | |
| | CCCCCATCAAGGACTTCGGCGGCTTCAACTTCAGCCAGATCCTGCCCG | |
| | ACCCCAGCAAGCCCAGCAAGAGGAGCCCCATCGAGGACCTGCTGTTCA | |
| | ACAAGGTGACCCTGGCCGACGCCGGCTTCATCAAGCAGTACGGCGACT | |
| | GCCTGGGCGACATCGCCGCCAGGGACCTGATCTGCGCCCAGAAGTTC | |
| | AACGGCCTGACCGTGCTGCCCCCCCTGCTGACCGACGAGATGATCGC | |
| | CCAGTACACCAGCGCCCTGCTGGCCGGCACCATCACCAGCGGCTGGA | |
| | CCTTCGGCGCCGGCCCCGCCCTGCAGATCCCCTTCCCCATGCAGATG | |
| | GCCTACAGGTTCAACGGCATCGGCGTGACCCAGAACGTGCTGTACGAG | |
| | AACCAGAAGCTGATCGCCAACCAGTTCAACAGCGCCATCGGCAAGATC | |
| | CAGGACAGCCTGAGCAGCACCCCCAGCGCCCTGGGCAAGCTGCAGGA | |
| | CGTGGTGAACCAGAACGCCCAGGCCCTGAACACCCTGGTGAAGCAGC | |
| | TGAGCAGCAACTTCGGCGCCATCAGCAGCGTGCTGAACGACATCCTGA | |
| | GCAGGCTGGACCCCCCCGAGGCCGAGGTGCAGATCGACAGGCTGATC | |
| | ACCGGCAGGCTGCAGAGCCTGCAGACCTACGTGACCCAGCAGCTGAT | |
| | CAGGGCCGCCGAGATCAGGGCCAGCGCCAACCTGGCCGCCACCAAGA | |
| | TGAGCGAGTGCGTGCTGGGCCAGAGCAAGAGGGTGGACTTCTGCGGC | |
| | AAGGGCTACCACCTGATGAGCTTCCCCCAGAGCGCCCCCCACGGCGT | |
| | GGTGTTCCTGCACGTGACCTACGTGCCCGCCCAGGAGAAGAACTTCAC | |
| | CACCGCCCCCGCCATCTGCCACGACGGCAAGGCCCACTTCCCCAGGG | |
| | AGGGCGTGTTCGTGAGCAACGGCACCCACTGGTTCGTGACCCAGAGG | |
| | AACTTCTACGAGCCCCAGATCATCACCACCGACAACACCTTCGTGAGC | |
| | GGCAACTGCGACGTGGTGATCGGCATCGTGAACAACACCGTGTACGAC | |
| | CCCCTGCAGCCCGAGCTGGACAGCTTCAAGGAGGAGCTGGACAAGTA | |
| | CTTCAAGAACCACACCAGCCCCGACGTGGACCTGGGCGACATCAGCG | |
| | GCATCAACGCCAGCGTGGTGAACATCCAGAAGGAGATCGACAGGCTGA | |
| | ACGAGGTGGCCAAGAACCTGAACGAGAGCCTGATCGACCTGCAGGAG | |
| | CTGGGCAAGTACGAGCAGTACATCAAGTGGCCCTGGTACATCTGGCTG | |
| | GGCTTCATCGCCGGCCTGATCGCCATCGTGATGGTGACCATCATGCTG | |
| | TGCTGCATGACCAGCTGCTGCAGCTGCCTGAAGGGCTGCTGCAGCTGC | |
| | GGCAGCTGCTGCAAGTTCGACGAGGACGACAGCGAGCCCGTGCTGAA | |
| | GGGCGTGAAGCTGCACTACACCTGATGACTCGAGCTGGTACTGCATGC | |
| | ACGCAATGCTAGCTGCCCCTTTCCCGTCCTGGGTACCCCGAGTCTCCC | |
| | CCGACCTCGGGTCCCAGGTATGCTCCCACCTCCACCTGCCCCACTCAC | |
| | CACCTCTGCTAGTTCCAGACACCTCCCAAGCACGCAGCAATGCAGCTC | |
| | AAAACGCTTAGCCTAGCCACACCCCCACGGGAAACAGCAGTGATTAAC | |
| | CTTTAGCAATAAACGAAAGTTTAACTAAGCTATACTAACCCCAGGGTTG | |
| | GTCAATTTCGTGCCAGCCACACCCTGGAGCTAGCAAAAAAAA | |
| 37 | TAGTTATTAATAGTAATCAATTACGGGGTCATTAGTTCATAGCCCATATA | 322 |
| Pan-CoV | TGGAGTTCCGCGTTACATAACTTACGGTAAATGGCCCGCCTGGCTGAC | |
| rAdv5-CMV- | CGCCCAACGACCCCCGCCCATTGACGTCAATAATGACGTATGTTCCCAT | |
| Two Proline | AGTAACGCCAATAGGGACTTTCCATTGACGTCAATGGGTGGAGTATTTA | |
| substitution | CGGTAAACTGCCCACTTGGCAGTACATCAAGTGTATCATATGCCAAGTA | |
| | CGCCCCCTATTGACGTCAATGACGGTAAATGGCCCGCCTGGCATTATG | |
| | CCCAGTACATGACCTTATGGGACTTTCCTACTTGGCAGTACATCTACGT | |
| | ATTAGTCATCGCTATTACCATGGTGATGCGGTTTTGGCAGTACATCAAT | |
| | GGGCGTGGATAGCGGTTTGACTCACGGGGATTTCCAAGTCTCCACCCC | |
| | ATTGACGTCAATGGGAGTTTGTTTTGGCACCAAAATCAACGGGACTTTC | |
| | CAAAATGTCGTAACAACTCCGCCCCATTGACGCAAATGGGCGGTAGGC | |
| | GTGTACGGTGGGAGGTCTATATAAGCAGAGCTGGTTTAGTGAACCGTC | |
| | AGATCGAGAATAAACTAGTATTCTTCTGGTCCCCACAGACTCAGAGAGA | |
| | ACCCGCCACCATGTTCGTGTTCCTGGTGCTGCTGCCCCTGGTGAGCAG | |
| | CCAGTGCGTGAACCTGACCACCAGGACCCAGCTGCCCCCCGCCTACA | |
| | CCAACAGCTTCACCAGGGGCGTGTACTACCCCGACAAGGTGTTCAGGA | |
| | GCAGCGTGCTGCACAGCACCCAGGACCTGTTCCTGCCCTTCTTCAGCA | |
| | ACGTGACCTGGTTCCACGCCATCCACGTGAGCGGCACCAACGGCACCA | |
| | AGAGGTTCGACAACCCCGTGCTGCCCTTCAACGACGGCGTGTACTTCG | |
| | CCAGCACCGAGAAGAGCAACATCATCAGGGGCTGGATCTTCGGCACCA | |
| | CCCTGGACAGCAAGACCCAGAGCCTGCTGATCGTGAACAACGCCACCA | |
| | ACGTGGTGATCAAGGTGTGCGAGTTCCAGTTCTGCAACGACCCCTTCC | |
| | TGGGCGTGTACTACCACAAGAACAACAAGAGCTGGATGGAGAGCGAGT | |
| | TCAGGGTGTACAGCAGCGCCAACAACTGCACCTTCGAGTACGTGAGCC | |
| | AGCCCTTCCTGATGGACCTGGAGGGCAAGCAGGGCAACTTCAAGAACC | |
| | TGAGGGAGTTCGTGTTCAAGAACATCGACGGCTACTTCAAGATCTACAG | |
| | CAAGCACACCCCCATCAACCTGGTGAGGGACCTGCCCCAGGGCTTCAG | |
| | CGCCCTGGAGCCCCTGGTGGACCTGCCCATCGGCATCAACATCACCAG | |
| | GTTCCAGACCCTGCTGGCCCTGCACAGGAGCTACCTGACCCCCGGCG | |
| | ACAGCAGCAGCGGCTGGACCGCCGGCGCCGCCGCCTACTACGTGGGC | |
| | TACCTGCAGCCCAGGACCTTCCTGCTGAAGTACAACGAGAACGGCACC | |
| | ATCACCGACGCCGTGGACTGCGCCCTGGACCCCCTGAGCGAGACCAA | |
| | GTGCACCCTGAAGAGCTTCACCGTGGAGAAGGGCATCTACCAGACCAG | |
| | CAACTTCAGGGTGCAGCCCACCGAGAGCATCGTGAGGTTCCCCAACAT | |
| | CACCAACCTGTGCCCCTTCGGCGAGGTGTTCAACGCCACCAGGTTCGC | |
| | CAGCGTGTACGCCTGGAACAGGAAGAGGATCAGCAACTGCGTGGCCG | |
| | ACTACAGCGTGCTGTACAACAGCGCCAGCTTCAGCACCTTCAAGTGCT | |
| | ACGGCGTGAGCCCCACCAAGCTGAACGACCTGTGCTTCACCAACGTGT | |
| | ACGCCGACAGCTTCGTGATCAGGGGCGACGAGGTGAGGCAGATCGCC | |
| | CCCGGCCAGACCGGCAAGATCGCCGACTACAACTACAAGCTGCCCGA | |
| | CGACTTCACCGGCTGCGTGATCGCCTGGAACAGCAACAACCTGGACAG | |
| | CAAGGTGGGCGGCAACTACAACTACCTGTACAGGCTGTTCAGGAAGAG | |
| | CAACCTGAAGCCCTTCGAGAGGGACATCAGCACCGAGATCTACCAGGC | |
| | CGGCAGCACCCCCTGCAACGGCGTGGAGGGCTTCAACTGCTACTTCCC | |
| | CCTGCAGAGCTACGGCTTCCAGCCCACCAACGGCGTGGGCTACCAGC | |
| | CCTACAGGGTGGTGGTGCTGAGCTTCGAGCTGCTGCACGCCCCCGCC | |
| | ACCGTGTGCGGCCCCAAGAAGAGCACCAACCTGGTGAAGAACAAGTGC | |
| | GTGAACTTCAACTTCAACGGCCTGACCGGCACCGGCGTGCTGACCGAG | |
| | AGCAACAAGAAGTTCCTGCCCTTCCAGCAGTTCGGCAGGGACATCGCC | |
| | GACACCACCGACGCCGTGAGGGACCCCCAGACCCTGGAGATCCTGGA | |
| | CATCACCCCCTGCAGCTTCGGCGGCGTGAGCGTGATCACCCCCGGCA | |
| | CCAACACCAGCAACCAGGTGGCCGTGCTGTACCAGGACGTGAACTGCA | |
| | CCGAGGTGCCCGTGGCCATCCACGCCGACCAGCTGACCCCCACCTGG | |
| | AGGGTGTACAGCACCGGCAGCAACGTGTTCCAGACCAGGGCCGGCTG | |
| | CCTGATCGGCGCCGAGCACGTGAACAACAGCTACGAGTGCGACATCCC | |
| | CATCGGCGCCGGCATCTGCGCCAGCTACCAGACCCAGACCAACAGCC | |
| | CCAGGAGGGCCAGGAGCGTGGCCAGCCAGAGCATCATCGCCTACACC | |
| | ATGAGCCTGGGCGCCGAGAACAGCGTGGCCTACAGCAACAACAGCAT | |
| | CGCCATCCCCACCAACTTCACCATCAGCGTGACCACCGAGATCCTGCC | |
| | CGTGAGCATGACCAAGACCAGCGTGGACTGCACCATGTACATCTGCGG | |
| | CGACAGCACCGAGTGCAGCAACCTGCTGCTGCAGTACGGCAGCTTCTG | |
| | CACCCAGCTGAACAGGGCCCTGACCGGCATCGCCGTGGAGCAGGACA | |
| | AGAACACCCAGGAGGTGTTCGCCCAGGTGAAGCAGATCTACAAGACCC | |
| | CCCCCATCAAGGACTTCGGCGGCTTCAACTTCAGCCAGATCCTGCCCG | |
| | ACCCCAGCAAGCCCAGCAAGAGGAGCTTCATCGAGGACCTGCTGTTCA | |
| | ACAAGGTGACCCTGGCCGACGCCGGCTTCATCAAGCAGTACGGCGACT | |
| | GCCTGGGCGACATCGCCGCCAGGGACCTGATCTGCGCCCAGAAGTTC | |
| | AACGGCCTGACCGTGCTGCCCCCCCTGCTGACCGACGAGATGATCGC | |
| | CCAGTACACCAGCGCCCTGCTGGCCGGCACCATCACCAGCGGCTGGA | |
| | CCTTCGGCGCCGGCGCCGCCCTGCAGATCCCCTTCGCCATGCAGATG | |
| | GCCTACAGGTTCAACGGCATCGGCGTGACCCAGAACGTGCTGTACGAG | |
| | AACCAGAAGCTGATCGCCAACCAGTTCAACAGCGCCATCGGCAAGATC | |
| | CAGGACAGCCTGAGCAGCACCGCCAGCGCCCTGGGCAAGCTGCAGGA | |
| | CGTGGTGAACCAGAACGCCCAGGCCCTGAACACCCTGGTGAAGCAGC | |
| | TGAGCAGCAACTTCGGCGCCATCAGCAGCGTGCTGAACGACATCCTGA | |
| | GCAGGCTGGACCCCCCCGAGGCCGAGGTGCAGATCGACAGGCTGATC | |
| | ACCGGCAGGCTGCAGAGCCTGCAGACCTACGTGACCCAGCAGCTGAT | |
| | CAGGGCCGCCGAGATCAGGGCCAGCGCCAACCTGGCCGCCACCAAGA | |
| | TGAGCGAGTGCGTGCTGGGCCAGAGCAAGAGGGTGGACTTCTGCGGC | |
| | AAGGGCTACCACCTGATGAGCTTCCCCCAGAGCGCCCCCCACGGCGT | |
| | GGTGTTCCTGCACGTGACCTACGTGCCCGCCCAGGAGAAGAACTTCAC | |
| | CACCGCCCCCGCCATCTGCCACGACGGCAAGGCCCACTTCCCCAGGG | |
| | AGGGCGTGTTCGTGAGCAACGGCACCCACTGGTTCGTGACCCAGAGG | |
| | AACTTCTACGAGCCCCAGATCATCACCACCGACAACACCTTCGTGAGC | |
| | GGCAACTGCGACGTGGTGATCGGCATCGTGAACAACACCGTGTACGAC | |
| | CCCCTGCAGCCCGAGCTGGACAGCTTCAAGGAGGAGCTGGACAAGTA | |
| | CTTCAAGAACCACACCAGCCCCGACGTGGACCTGGGCGACATCAGCG | |
| | GCATCAACGCCAGCGTGGTGAACATCCAGAAGGAGATCGACAGGCTGA | |
| | ACGAGGTGGCCAAGAACCTGAACGAGAGCCTGATCGACCTGCAGGAG | |
| | CTGGGCAAGTACGAGCAGTACATCAAGTGGCCCTGGTACATCTGGCTG | |
| | GGCTTCATCGCCGGCCTGATCGCCATCGTGATGGTGACCATCATGCTG | |
| | TGCTGCATGACCAGCTGCTGCAGCTGCCTGAAGGGCTGCTGCAGCTGC | |
| | GGCAGCTGCTGCAAGTTCGACGAGGACGACAGCGAGCCCGTGCTGAA | |
| | GGGCGTGAAGCTGCACTACACCTGATGACTCGAGCTGGTACTGCATGC | |
| | ACGCAATGCTAGCTGCCCCTTTCCCGTCCTGGGTACCCCGAGTCTCCC | |
| | CCGACCTCGGGTCCCAGGTATGCTCCCACCTCCACCTGCCCCACTCAC | |
| | CACCTCTGCTAGTTCCAGACACCTCCCAAGCACGCAGCAATGCAGCTC | |
| | AAAACGCTTAGCCTAGCCACACCCCCACGGGAAACAGCAGTGATTAAC | |
| | CTTTAGCAATAAACGAAAGTTTAACTAAGCTATACTAACCCCAGGGTTG | |
| | GTCAATTTCGTGCCAGCCACACCCTGGAGCTAGCAAAAAAAA | |
| 38 | TAGTTATTAATAGTAATCAATTACGGGGTCATTAGTTCATAGCCCATATA | 323 |
| Pan-CoV | TGGAGTTCCGCGTTACATAACTTACGGTAAATGGCCCGCCTGGCTGAC | |
| rAdv5-CMV- | CGCCCAACGACCCCCGCCCATTGACGTCAATAATGACGTATGTTCCCAT | |
| Nucleocapsid | AGTAACGCCAATAGGGACTTTCCATTGACGTCAATGGGTGGAGTATTTA | |
| | CGGTAAACTGCCCACTTGGCAGTACATCAAGTGTATCATATGCCAAGTA | |
| | CGCCCCCTATTGACGTCAATGACGGTAAATGGCCCGCCTGGCATTATG | |
| | CCCAGTACATGACCTTATGGGACTTTCCTACTTGGCAGTACATCTACGT | |
| | ATTAGTCATCGCTATTACCATGGTGATGCGGTTTTGGCAGTACATCAAT | |
| | GGGCGTGGATAGCGGTTTGACTCACGGGGATTTCCAAGTCTCCACCCC | |
| | ATTGACGTCAATGGGAGTTTGTTTTGGCACCAAAATCAACGGGACTTTC | |
| | CAAAATGTCGTAACAACTCCGCCCCATTGACGCAAATGGGCGGTAGGC | |
| | GTGTACGGTGGGAGGTCTATATAAGCAGAGCTGGTTTAGTGAACCGTC | |
| | AGATCGAGAATAAACTAGTATTCTTCTGGTCCCCACAGACTCAGAGAGA | |
| | ACCCGCCACCATGAGCGACAACGGCCCCCAGAACCAGAGGAACGCCC | |
| | CCAGGATCACCTTCGGCGGCCCCAGCGACAGCACCGGCAGCAACCAG | |
| | AACGGCGAGAGGAGCGGCGCCAGGAGCAAGCAGAGGAGGCCCCAGG | |
| | GCCTGCCCAACAACACCGCCAGCTGGTTCACCGCCCTGACCCAGCAC | |
| | GGCAAGGAGGACCTGAAGTTCCCCAGGGGCCAGGGCGTGCCCATCAA | |
| | CACCAACAGCAGCCCCGACGACCAGATCGGCTACTACAGGAGGGCCA | |
| | CCAGGAGGATCAGGGGCGGCGACGGCAAGATGAAGGACCTGAGCCCC | |
| | AGGTGGTACTTCTACTACCTGGGCACCGGCCCCGAGGCCGGCCTGCC | |
| | CTACGGCGCCAACAAGGACGGCATCATCTGGGTGGCCACCGAGGGCG | |
| | CCCTGAACACCCCCAAGGACCACATCGGCACCAGGAACCCCGCCAACA | |
| | ACGCCGCCATCGTGCTGCAGCTGCCCCAGGGCACCACCCTGCCCAAG | |
| | GGCTTCTACGCCGAGGGCAGCAGGGGCGGCAGCCAGGCCAGCAGCA | |
| | GGAGCAGCAGCAGGAGCAGGAACAGCAGCAGGAACAGCACCCCCGGC | |
| | AGCAGCAGGGGCACCAGCCCCGCCAGGATGGCCGGCAACGGCGGCG | |
| | ACGCCGCCCTGGCCCTGCTGCTGCTGGACAGGCTGAACCAGCTGGAG | |
| | AGCAAGATGAGCGGCAAGGGCCAGCAGCAGCAGGGCCAGACCGTGAC | |
| | CAAGAAGAGCGCCGCCGAGGCCAGCAAGAAGCCCAGGCAGAAGAGGA | |
| | CCGCCACCAAGGCCTACAACGTGACCCAGGCCTTCGGCAGGAGGGGC | |
| | CCCGAGCAGACCCAGGGCAACTTCGGCGACCAGGAGCTGATCAGGCA | |
| | GGGCACCGACTACAAGCACTGGCCCCAGATCGCCCAGTTCGCCCCCA | |
| | GCGCCAGCGCCTTCTTCGGCATGAGCAGGATCGGCATGGAGGTGACC | |
| | CCCAGCGGCACCTGGCTGACCTACACCGGCGCCATCAAGCTGGACGA | |
| | CAAGGACCCCAACTTCAAGGACCAGGTGATCCTGCTGAACAAGCACAT | |
| | CGACGCCTACAAGACCTTCCCCCCCACCGAGCCCAAGAAGGACAAGAA | |
| | GAAGAAGGCCGACGAGACCCAGGCCCTGCCCCAGAGGCAGAAGAAGC | |
| | AGCAGACCGTGACCCTGCTGCCCGCCGCCGACCTGGACGACTTCAGC | |
| | AAGCAGCTGCAGCAGAGCATGAGCAGCGCCGACAGCACCCAGGCCTG | |
| | ATGACTCGAGCTGGTACTGCATGCACGCAATGCTAGCTGCCCCTTTCC | |
| | CGTCCTGGGTACCCCGAGTCTCCCCCGACCTCGGGTCCCAGGTATGCT | |
| | CCCACCTCCACCTGCCCCACTCACCACCTCTGCTAGTTCCAGACACCT | |
| | CCCAAGCACGCAGCAATGCAGCTCAAAACGCTTAGCCTAGCCACACCC | |
| | CCACGGGAAACAGCAGTGATTAACCTTTAGCAATAAACGAAAGTTTAAC | |
| | TAAGCTATACTAACCCCAGGGTTGGTCAATTTCGTGCCAGCCACACCCT | |
| | GGAGCTAGCAAAAAAAA | |
| 39 | TAGTTATTAATAGTAATCAATTACGGGGTCATTAGTTCATAGCCCATATA | 324 |
| Pan-CoV | TGGAGTTCCGCGTTACATAACTTACGGTAAATGGCCCGCCTGGCTGAC | |
| rAdv5-CMV- | CGCCCAACGACCCCCGCCCATTGACGTCAATAATGACGTATGTTCCCAT | |
| ORF1a/b | AGTAACGCCAATAGGGACTTTCCATTGACGTCAATGGGTGGAGTATTTA | |
| | CGGTAAACTGCCCACTTGGCAGTACATCAAGTGTATCATATGCCAAGTA | |
| | CGCCCCCTATTGACGTCAATGACGGTAAATGGCCCGCCTGGCATTATG | |
| | CCCAGTACATGACCTTATGGGACTTTCCTACTTGGCAGTACATCTACGT | |
| | ATTAGTCATCGCTATTACCATGGTGATGCGGTTTTGGCAGTACATCAAT | |
| | GGGCGTGGATAGCGGTTTGACTCACGGGGATTTCCAAGTCTCCACCCC | |
| | ATTGACGTCAATGGGAGTTTGTTTTGGCACCAAAATCAACGGGACTTTC | |
| | CAAAATGTCGTAACAACTCCGCCCCATTGACGCAAATGGGCGGTAGGC | |
| | GTGTACGGTGGGAGGTCTATATAAGCAGAGCTGGTTTAGTGAACCGTC | |
| | AGATCGAGAATAAACTAGTATTCTTCTGGTCCCCACAGACTCAGAGAGA | |
| | ACCCGCCACCATGGACCAGGAACCCCGCCTGGAGGAAGGCCGTGTTC | |
| | ATCAGCCCCTACAACAGCCAGAACGCCGTGGCCAGCAAGATCCTGGGC | |
| | CTGCCCACCCAGACCGTGGACAGCAGCCAGGGCAGCGAGTACGACTA | |
| | CGTGATCTTCACCCAGACCACCGAGACCGCCCACAGCTGCAACGTGAA | |
| | CAGGTTCAACGTGGCCATCACCAGGGCCAAGGTGGGCATCCTGTGCAT | |
| | CATGAGCGACAGGGACCTGTACGACAAGCTGCAGTTCACCAGCCTGGA | |
| | GATCCCCAGGAGGAACGTGGCCACCCTGCAGGCCGAGAACGTGACCG | |
| | GCCTGTTCAAGGACTGCAGCAAGGTGATCACCGGCCTGCACCCCACCC | |
| | AGGCCCCCACCCACCTGAGCGTGGACACCAAGTTCAAGACCGAGGGC | |
| | CTGTGCGTGGACATCCCCGGCATCCCCAAGGACATGACCTACAGGAGG | |
| | CTGATCAGCATGATGGGCTTCAAGATGAACTACCAGGTGAACGGCTAC | |
| | CCCAACATGTTCATCACCAGGGAGGAGGCCATCAGGCACGTGAGGGC | |
| | CTGGATCGGCTTCGACGTGGAGGGCTGCCACGCCACCAGGGAGGCCG | |
| | TGGGCACCAACCTGCCCCTGCAGCTGGGCTTCAGCACCGGCGTGAAC | |
| | CTGGTGGCCGTGCCCACCGGCTACGTGGACACCCCCAACAACACCGA | |
| | CTTCAGCAGGGTGAGCGCCAAGCCCCCCCCCGGCGACCAGTTCAAGC | |
| | ACCTGATCCCCCTGATGTACAAGGGCCTGCCCTGGAACGTGGTGAGGA | |
| | TCAAGATCGTGCAGATGCTGAGCGACACCCTGAAGAACCTGAGCGACA | |
| | GGGTGGTGTTCGTGCTGTGGGCCCACGGCTTCGAGCTGACCAGCATG | |
| | AAGTACTTCGTGAAGATCGGCCCCGAGAGGACCTGCTGCCTGTGCGAC | |
| | AGGAGGGCCACCTGCTTCAGCACCGCCAGCGACACCTACGCCTGCTG | |
| | GCACCACAGCATCGGCTTCGACTACGTGTACAACCCCTTCATGATCGA | |
| | CGTGCAGCAGTGGGGCTTCACCGGCAACCTGCAGAGCAACCACGACC | |
| | TGTACTGCCAGGTGCACGGCAACGCCCACGTGGCCAGCTGCGACGCC | |
| | ATCATGACCAGGTGCCTGGCCGTGCACGAGTGCTTCGTGAAGAGGGTG | |
| | GACTGGACCATCGAGTACCCCATCATCGGCGACGAGCTGAAGATCAAC | |
| | GCCGCCTGCAGGAAGGTGCAGCACATGGTGGTGAAGGCCGCCCTGCT | |
| | GGCCGACAAGTTCCCCGTGCTGCACGACATCGGCAACCCCAAGGCCAT | |
| | CAAGTGCGTGCCCCAGGCCGACGTGGAGTGGAAGTTCTACGACGCCC | |
| | AGCCCTGCAGCGACAAGGCCTACAAGATCGAGGAGCTGTTCTACAGCT | |
| | ACGCCACCCACAGCGACAAGTTCACCGACGGCGTGTGCCTGTTCTGGA | |
| | ACTGCAACGTGGACAGGTACCCCGCCAACAGCATCGTGTGCAGGTTCG | |
| | ACACCAGGGTGCTGAGCAACCTGAACCTGCCCGGCTGCGACGGCGGC | |
| | AGCCTGTACGTGAACAAGCACGCCTTCCACACCCCCGCCTTCGACAAG | |
| | AGCGCCTTCGTGAACCTGAAGCAGCTGCCCTTCTTCTACTACAGCGAC | |
| | AGCCCCTGCGAGAGCCACGGCAAGCAGGTGGTGAGCGACATCGACTA | |
| | CGTGCCCCTGAAGAGCGCCACCTGCATCACCAGGTGCAACCTGGGCG | |
| | GCGCCGTGTGCAGGCACCACGCCAACGAGTACAGGCTGTACCTGGAC | |
| | GCCTACAACATGATGATCAGCGCCGGCTTCAGCCTGTGGGTGTACAAG | |
| | CAGTTCGACACCTACAACCTGTGGAACACCTTCACCAGGCTGCAGAGC | |
| | CTGGAGAACGTGGCCTTCAACGTGGTGAACAAGGGCCACTTCGACGGC | |
| | CAGCAGGGCGAGGTGCCCGTGAGCATCATCAACAACACCGTGTACACC | |
| | AAGGTGGACGGCGTGGACGTGGAGCTGTTCGAGAACAAGACCACCCT | |
| | GCCCGTGAACGTGGCCTTCGAGCTGTGGGCCAAGAGGAACATCAAGC | |
| | CCGTGCCCGAGGTGAAGATCCTGAACAACCTGGGCGTGGACATCGCC | |
| | GCCAACACCGTGATCTGGGACTACAAGAGGGACGCCCCCGCCCACAT | |
| | CAGCACCATCGGCGTGTGCAGCATGACCGACATCGCCAAGAAGCCCAC | |
| | CGAGACCATCTGCGCCCCCCTGACCGTGTTCTTCGACGGCAGGGTGG | |
| | ACGGCCAGGTGGACCTGTTCAGGAACGCCAGGAACGGCGTGCTGATC | |
| | ACCGAGGGCAGCGTGAAGGGCCTGCAGCCCAGCGTGGGCCCCAAGCA | |
| | GGCCAGCCTGAACGGCGTGACCCTGATCGGCGAGGCCGTGAAGACCC | |
| | AGTTCAACTACTACAAGAAGGTGGACGGCGTGGTGCAGCAGCTGCCCG | |
| | AGACCTACTTCACCCAGAGCAGGAACCTGCAGGAGTTCAAGCCCAGGA | |
| | GCCAGATGGAGATCGACTTCCTGGAGCTGGCCATGGACGAGTTCATCG | |
| | AGAGGTACAAGCTGGAGGGCTACGCCTTCGAGCACATCGTGTACGGC | |
| | GACTTCAGCCACAGCCAGCTGGGCGGCCTGCACCTGCTGATCGGCCT | |
| | GGCCAAGAGGTTCAAGGAGAGCCCCTTCGAGCTGGAGGACTTCATCCC | |
| | CATGGACAGCACCGTGAAGAACTACTTCATCACCGACGCCCAGACCGG | |
| | CAGCAGCAAGTGCGTGTGCAGCGTGATCGACCTGCTGCTGGACGACTT | |
| | CGTGGAGATCATCAAGAGCCAGGACCTGAGCGTGGTGAGCAAGGTGG | |
| | TGAAGGTGACCATCGACTACACCGAGATCAGCTTCATGCTGTGGTGCA | |
| | AGGACGGCCACGTGGAGACCTTCTACCCCAAGCTGCAGAGCAGCCAG | |
| | GCCTGGCAGCCCGGCGTGGCCATGCCCAACCTGTACAAGATGCAGAG | |
| | GATGCTGCTGGAGAAGTGCGACCTGCAGAACTACGGCGACAGCGCCA | |
| | CCCTGCCCAAGGGCATCATGATGAACGTGGCCAAGTACACCCAGCTGT | |
| | GCCAGTACCTGAACACCCTGACCCTGGCCGTGCCCTACAACATGAGGG | |
| | TGATCCACTTCGGCGCCGGCAGCGACAAGGGCGTGGCCCCCGGCACC | |
| | GCCGTGCTGAGGCAGTGGCTGCCCACCGGCACCCTGCTGGTGGACAG | |
| | CGACCTGAACGACTTCGTGAGCGACGCCGACAGCACCCTGATCGGCG | |
| | ACTGCGCCACCGTGCACACCGCCAACAAGTGGGACCTGATCATCAGCG | |
| | ACATGTACGACCCCAAGACCAAGAACGTGACCAAGGAGAACGACAGCA | |
| | AGGAGGGCTTCTTCACCTACATCTGCGGCTTCATCCAGCAGAAGCTGG | |
| | CCCTGGGCGGCAGCGTGGCCATCAAGATCACCGAGCACAGCTGGAAC | |
| | GCCGACCTGTACAAGCTGATGGGCCACTTCGCCTGGTGGACCGCCTTC | |
| | GTGACCAACGTGAACGCCAGCAGCAGCGAGGCCTTCCTGATCGGCTG | |
| | CAACTACCTGGGCAAGCCCAGGGAGCAGATCGACGGCTACGTGATGC | |
| | ACGCCAACTACATCTTCTGGAGGAACACCAACCCCATCCAGCTGAGCA | |
| | GCTACAGCCTGTTCGACATGAGCAAGTTCCCCCTGAAGCTTGATGACTC | |
| | GAGCTGGTACTGCATGCACGCAATGCTAGCTGCCCCTTTCCCGTCCTG | |
| | GGTACCCCGAGTCTCCCCCGACCTCGGGTCCCAGGTATGCTCCCACCT | |
| | CCACCTGCCCCACTCACCACCTCTGCTAGTTCCAGACACCTCCCAAGC | |
| | ACGCAGCAATGCAGCTCAAAACGCTTAGCCTAGCCACACCCCCACGGG | |
| | AAACAGCAGTGATTAACCTTTAGCAATAAACGAAAGTTTAACTAAGCTAT | |
| | ACTAACCCCAGGGTTGGTCAATTTCGTGCCAGCCACACCCTGGAGCTA | |
| | GCAAAAAAAA | |
| 40 | TAGTTATTAATAGTAATCAATTACGGGGTCATTAGTTCATAGCCCATATA | 325 |
| Pan-CoV | TGGAGTTCCGCGTTACATAACTTACGGTAAATGGCCCGCCTGGCTGAC | |
| rAdv5-CMV- | CGCCCAACGACCCCCGCCCATTGACGTCAATAATGACGTATGTTCCCAT | |
| Envelope | AGTAACGCCAATAGGGACTTTCCATTGACGTCAATGGGTGGAGTATTTA | |
| | CGGTAAACTGCCCACTTGGCAGTACATCAAGTGTATCATATGCCAAGTA | |
| | CGCCCCCTATTGACGTCAATGACGGTAAATGGCCCGCCTGGCATTATG | |
| | CCCAGTACATGACCTTATGGGACTTTCCTACTTGGCAGTACATCTACGT | |
| | ATTAGTCATCGCTATTACCATGGTGATGCGGTTTTGGCAGTACATCAAT | |
| | GGGCGTGGATAGCGGTTTGACTCACGGGGATTTCCAAGTCTCCACCCC | |
| | ATTGACGTCAATGGGAGTTTGTTTTGGCACCAAAATCAACGGGACTTTC | |
| | CAAAATGTCGTAACAACTCCGCCCCATTGACGCAAATGGGCGGTAGGC | |
| | GTGTACGGTGGGAGGTCTATATAAGCAGAGCTGGTTTAGTGAACCGTC | |
| | AGATCGAGAATAAACTAGTATTCTTCTGGTCCCCACAGACTCAGAGAGA | |
| | ACCCGCCACCATGTACAGCTTCGTGAGCGAGGAGACCGGCACCCTGAT | |
| | CGTGAACAGCGTGCTGCTGTTCCTGGCCTTCGTGGTGTTCCTGCTGGT | |
| | GACCCTGGCCATCCTGACCGCCCTGAGGCTGTGCGCCTACTGCTGCAA | |
| | CATCGTGAACGTGAGCCTGGTGAAGCCCAGCTTCTACGTGTACAGCAG | |
| | GGTGAAGAACCTGAACAGCAGCAGGGTGCCCGACCTGCTGGTGTGAT | |
| | GACTCGAGCTGGTACTGCATGCACGCAATGCTAGCTGCCCCTTTCCCG | |
| | TCCTGGGTACCCCGAGTCTCCCCCGACCTCGGGTCCCAGGTATGCTCC | |
| | CACCTCCACCTGCCCCACTCACCACCTCTGCTAGTTCCAGACACCTCC | |
| | CAAGCACGCAGCAATGCAGCTCAAAACGCTTAGCCTAGCCACACCCCC | |
| | ACGGGAAACAGCAGTGATTAACCTTTAGCAATAAACGAAAGTTTAACTA | |
| | AGCTATACTAACCCCAGGGTTGGTCAATTTCGTGCCAGCCACACCCTG | |
| | GAGCTAGCAAAAAAAA | |
| 41 | TAGTTATTAATAGTAATCAATTACGGGGTCATTAGTTCATAGCCCATATA | 326 |
| Pan-CoV | TGGAGTTCCGCGTTACATAACTTACGGTAAATGGCCCGCCTGGCTGAC | |
| rAdv5-CMV- | CGCCCAACGACCCCCGCCCATTGACGTCAATAATGACGTATGTTCCCAT | |
| Membrane | AGTAACGCCAATAGGGACTTTCCATTGACGTCAATGGGTGGAGTATTTA | |
| | CGGTAAACTGCCCACTTGGCAGTACATCAAGTGTATCATATGCCAAGTA | |
| | CGCCCCCTATTGACGTCAATGACGGTAAATGGCCCGCCTGGCATTATG | |
| | CCCAGTACATGACCTTATGGGACTTTCCTACTTGGCAGTACATCTACGT | |
| | ATTAGTCATCGCTATTACCATGGTGATGCGGTTTTGGCAGTACATCAAT | |
| | GGGCGTGGATAGCGGTTTGACTCACGGGGATTTCCAAGTCTCCACCCC | |
| | ATTGACGTCAATGGGAGTTTGTTTTGGCACCAAAATCAACGGGACTTTC | |
| | CAAAATGTCGTAACAACTCCGCCCCATTGACGCAAATGGGCGGTAGGC | |
| | GTGTACGGTGGGAGGTCTATATAAGCAGAGCTGGTTTAGTGAACCGTC | |
| | AGATCGAGAATAAACTAGTATTCTTCTGGTCCCCACAGACTCAGAGAGA | |
| | ACCCGCCACCATGGCCGACAGCAACGGCACCATCACCGTGGAGGAGC | |
| | TGAAGAAGCTGCTGGAGCAGTGGAACCTGGTGATCGGCTTCCTGTTCC | |
| | TGACCTGGATCTGCCTGCTGCAGTTCGCCTACGCCAACAGGAACAGGT | |
| | TCCTGTACATCATCAAGCTGATCTTCCTGTGGCTGCTGTGGCCCGTGAC | |
| | CCTGGCCTGCTTCGTGCTGGCCGCCGTGTACAGGATCAACTGGATCAC | |
| | CGGCGGCATCGCCATCGCCATGGCCTGCCTGGTGGGCCTGATGTGGC | |
| | TGAGCTACTTCATCGCCAGCTTCAGGCTGTTCGCCAGGACCAGGAGCA | |
| | TGTGGAGCTTCAACCCCGAGACCAACATCCTGCTGAACGTGCCCCTGC | |
| | ACGGCACCATCCTGACCAGGCCCCTGCTGGAGAGCGAGCTGGTGATC | |
| | GGCGCCGTGATCCTGAGGGGCCACCTGAGGATCGCCGGCCACCACCT | |
| | GGGCAGGTGCGACATCAAGGACCTGCCCAAGGAGATCACCGTGGCCA | |
| | CCAGCAGGACCCTGAGCTACTACAAGCTGGGCGCCAGCCAGAGGGTG | |
| | GCCGGCGACAGCGGCTTCGCCGCCTACAGCAGGTACAGGATCGGCAA | |
| | CTACAAGCTGAACACCGACCACAGCAGCAGCAGCGACAACATCGCCCT | |
| | GCTGGTGCAGTGATGACTCGAGCTGGTACTGCATGCACGCAATGCTAG | |
| | CTGCCCCTTTCCCGTCCTGGGTACCCCGAGTCTCCCCCGACCTCGGGT | |
| | CCCAGGTATGCTCCCACCTCCACCTGCCCCACTCACCACCTCTGCTAG | |
| | TTCCAGACACCTCCCAAGCACGCAGCAATGCAGCTCAAAACGCTTAGC | |
| | CTAGCCACACCCCCACGGGAAACAGCAGTGATTAACCTTTAGCAATAAA | |
| | CGAAAGTTTAACTAAGCTATACTAACCCCAGGGTTGGTCAATTTCGTGC | |
| | CAGCCACACCCTGGAGCTAGCAAAAAAAA | |
| 42 | TAGTTATTAATAGTAATCAATTACGGGGTCATTAGTTCATAGCCCATATA | 327 |
| Pan-CoV | TGGAGTTCCGCGTTACATAACTTACGGTAAATGGCCCGCCTGGCTGAC | |
| rAdv5-CMV- | CGCCCAACGACCCCCGCCCATTGACGTCAATAATGACGTATGTTCCCAT | |
| ORF7a | AGTAACGCCAATAGGGACTTTCCATTGACGTCAATGGGTGGAGTATTTA | |
| | CGGTAAACTGCCCACTTGGCAGTACATCAAGTGTATCATATGCCAAGTA | |
| | CGCCCCCTATTGACGTCAATGACGGTAAATGGCCCGCCTGGCATTATG | |
| | CCCAGTACATGACCTTATGGGACTTTCCTACTTGGCAGTACATCTACGT | |
| | ATTAGTCATCGCTATTACCATGGTGATGCGGTTTTGGCAGTACATCAAT | |
| | GGGCGTGGATAGCGGTTTGACTCACGGGGATTTCCAAGTCTCCACCCC | |
| | ATTGACGTCAATGGGAGTTTGTTTTGGCACCAAAATCAACGGGACTTTC | |
| | CAAAATGTCGTAACAACTCCGCCCCATTGACGCAAATGGGCGGTAGGC | |
| | GTGTACGGTGGGAGGTCTATATAAGCAGAGCTGGTTTAGTGAACCGTC | |
| | AGATCGAGAATAAACTAGTATTCTTCTGGTCCCCACAGACTCAGAGAGA | |
| | ACCCGCCACCATGAAGATCATCCTGTTCCTGGCCCTGATCACCCTGGC | |
| | CACCTGCGAGCTGTACCACTACCAGGAGTGCGTGAGGGGCACCACCG | |
| | TGTGATGACTCGAGCTGGTACTGCATGCACGCAATGCTAGCTGCCCCT | |
| | TTCCCGTCCTGGGTACCCCGAGTCTCCCCCGACCTCGGGTCCCAGGTA | |
| | TGCTCCCACCTCCACCTGCCCCACTCACCACCTCTGCTAGTTCCAGACA | |
| | CCTCCCAAGCACGCAGCAATGCAGCTCAAAACGCTTAGCCTAGCCACA | |
| | CCCCCACGGGAAACAGCAGTGATTAACCTTTAGCAATAAACGAAAGTTT | |
| | AACTAAGCTATACTAACCCCAGGGTTGGTCAATTTCGTGCCAGCCACAC | |
| | CCTGGAGCTAGCAAAAAAAA | |
| 43 | TAGTTATTAATAGTAATCAATTACGGGGTCATTAGTTCATAGCCCATATA | 328 |
| Pan-CoV | TGGAGTTCCGCGTTACATAACTTACGGTAAATGGCCCGCCTGGCTGAC | |
| rAdv5-CMV- | CGCCCAACGACCCCCGCCCATTGACGTCAATAATGACGTATGTTCCCAT | |
| NSP3 | AGTAACGCCAATAGGGACTTTCCATTGACGTCAATGGGTGGAGTATTTA | |
| | CGGTAAACTGCCCACTTGGCAGTACATCAAGTGTATCATATGCCAAGTA | |
| | CGCCCCCTATTGACGTCAATGACGGTAAATGGCCCGCCTGGCATTATG | |
| | CCCAGTACATGACCTTATGGGACTTTCCTACTTGGCAGTACATCTACGT | |
| | ATTAGTCATCGCTATTACCATGGTGATGCGGTTTTGGCAGTACATCAAT | |
| | GGGCGTGGATAGCGGTTTGACTCACGGGGATTTCCAAGTCTCCACCCC | |
| | ATTGACGTCAATGGGAGTTTGTTTTGGCACCAAAATCAACGGGACTTTC | |
| | CAAAATGTCGTAACAACTCCGCCCCATTGACGCAAATGGGCGGTAGGC | |
| | GTGTACGGTGGGAGGTCTATATAAGCAGAGCTGGTTTAGTGAACCGTC | |
| | AGATCGAGAATAAACTAGTATTCTTCTGGTCCCCACAGACTCAGAGAGA | |
| | ACCCGCCACCATGGCCCCCACCAAGGTGACCTTCGGCGACGACACCG | |
| | TGATCGAGGTGCAGGGCTACAAGAGCGTGAACATCACCTTCGAGCTGG | |
| | ACGAGAGGATCGACAAGGTGCTGAACGAGAAGTGCAGCGCCTACACC | |
| | GTGGAGCTGGGCACCGAGGTGAACGAGTTCGCCTGCGTGGTGGCCGA | |
| | CGCCGTGATCAAGACCCTGCAGCCCGTGAGCGAGCTGCTGACCCCCC | |
| | TGGGCATCGACCTGGACGAGTGGAGCATGGCCACCTACTACCTGTTCG | |
| | ACGAGAGCGGCGAGTTCAAGCTGGCCAGCCACATGTACTGCAGCTTCT | |
| | ACCCCCCCGACGAGGACGAGGAGGAGGGCGACTGCGAGGAGGAGGA | |
| | GTTCGAGCCCAGCACCCAGTACGAGTACGGCACCGAGGACGACTACC | |
| | AGGGCAAGCCCCTGGAGTTCGGCGCCACCAGCGCCGCCCTGCAGCCC | |
| | GAGGAGGAGCAGGAGGAGGACTGGCTGGACGACGACAGCCAGCAGA | |
| | CCGTGGGCCAGCAGGACGGCAGCGAGGACAACCAGACCACCACCATC | |
| | CAGACCATCGTGGAGGTGCAGCCCCAGCTGGAGATGGAGCTGACCCC | |
| | CGTGGTGCAGACCATCGAGGTGAACAGCTTCAGCGGCTACCTGAAGCT | |
| | GACCGACAACGTGTACATCAAGAACGCCGACATCGTGGAGGAGGCCAA | |
| | GAAGGTGAAGCCCACCGTGGTGGTGAACGCCGCCAACGTGTACCTGA | |
| | AGCACGGCGGCGGCGTGGCCGGCGCCCTGAACAAGGCCACCAACAAC | |
| | GCCATGCAGGTGGAGAGCGACGACTACATCGCCACCAACGGCCCCCT | |
| | GAAGGTGGGCGGCAGCTGCGTGCTGAGCGGCCACAACCTGGCCAAGC | |
| | ACTGCCTGCACGTGGTGGGCCCCAACGTGAACAAGGGCGAGGACATC | |
| | CAGCTGCTGAAGAGCGCCTACGAGAACTTCAACCAGCACGAGGTGCTG | |
| | CTGGCCCCCCTGCTGAGCGCCGGCATCTTCGGCGCCGACCCCATCCA | |
| | CAGCCTGAGGGTGTGCGTGGACACCGTGAGGACCAACGTGTACCTGG | |
| | CCGTGTTCGACAAGAACCTGTACGACAAGCTGGTGAGCAGCTTCCTGG | |
| | AGATGAAGAGCGAGAAGCAGGTGGAGCAGAAGATCGCCGAGATCCCC | |
| | AAGGAGGAGGTGAAGCCCTTCATCACCGAGAGCAAGCCCAGCGTGGA | |
| | GCAGAGGAAGCAGGACGACAAGAAGATCAAGGCCTGCGTGGAGGAGG | |
| | TGACCACCACCCTGGAGGAGACCAAGTTCCTGACCGAGAACCTGCTGC | |
| | TGTACATCGACATCAACGGCAACCTGCACCCCGACAGCGCCACCCTGG | |
| | TGAGCGACATCGACATCACCTTCCTGAAGAAGGACGCCCCCTACATCG | |
| | TGGGCGACGTGGTGCAGGAGGGCGTGCTGACCGCCGTGGTGATCCCC | |
| | ACCAAGAAGGCCGGCGGCACCACCGAGATGCTGGCCAAGGCCCTGAG | |
| | GAAGGTGCCCACCGACAACTACATCACCACCTACCCCGGCCAGGGCCT | |
| | GAACGGCTACACCGTGGAGGAGGCCAAGACCGTGCTGAAGAAGTGCA | |
| | AGAGCGCCTTCTACATCCTGCCCAGCATCATCAGCAACGAGAAGCAGG | |
| | AGATCCTGGGCACCGTGAGCTGGAACCTGAGGGAGATGCTGGCCCAC | |
| | GCCGAGGAGACCAGGAAGCTGATGCCCGTGTGCGTGGAGACCAAGGC | |
| | CATCGTGAGCACCATCCAGAGGAAGTACAAGGGCATCAAGATCCAGGA | |
| | GGGCGTGGTGGACTACGGCGCCAGGTTCTACTTCTACACCAGCAAGAC | |
| | CACCGTGGCCAGCCTGATCAACACCCTGAACGACCTGAACGAGACCCT | |
| | GGTGACCATGCCCCTGGGCTACGTGACCCACGGCCTGAACCTGGAGG | |
| | AGGCCGCCAGGTACATGAGGAGCCTGAAGGTGCCCGCCACCGTGAGC | |
| | GTGAGCAGCCCCGACGCCGTGACCGCCTACAACGGCTACCTGACCAG | |
| | CAGCAGCAAGACCCCCGAGGAGCACTTCATCGAGACCATCAGCCTGGC | |
| | CGGCAGCTACAAGGACTGGAGCTACAGCGGCCAGAGCACCCAGCTGG | |
| | GCATCGAGTTCCTGAAGAGGGGCGACAAGAGCGTGTACTACACCAGCA | |
| | ACCCCACCACCTTCCACCTGGACGGCGAGGTGATCACCTTCGACAACC | |
| | TGAAGACCCTGCTGAGCCTGAGGGAGGTGAGGACCATCAAGGTGTTCA | |
| | CCACCGTGGACAACATCAACCTGCACTGATGACTCGAGCTGGTACTGC | |
| | ATGCACGCAATGCTAGCTGCCCCTTTCCCGTCCTGGGTACCCCGAGTC | |
| | TCCCCCGACCTCGGGTCCCAGGTATGCTCCCACCTCCACCTGCCCCAC | |
| | TCACCACCTCTGCTAGTTCCAGACACCTCCCAAGCACGCAGCAATGCA | |
| | GCTCAAAACGCTTAGCCTAGCCACACCCCCACGGGAAACAGCAGTGAT | |
| | TAACCTTTAGCAATAAACGAAAGTTTAACTAAGCTATACTAACCCCAGGG | |
| | TTGGTCAATTTCGTGCCAGCCACACCCTGGAGCTAGCAAAAAAAA | |
| 44 | TAGTTATTAATAGTAATCAATTACGGGGTCATTAGTTCATAGCCCATATA | 329 |
| Pan-CoV | TGGAGTTCCGCGTTACATAACTTACGGTAAATGGCCCGCCTGGCTGAC | |
| rAdv5-CMV- | CGCCCAACGACCCCCGCCCATTGACGTCAATAATGACGTATGTTCCCAT | |
| NSP5 | AGTAACGCCAATAGGGACTTTCCATTGACGTCAATGGGTGGAGTATTTA | |
| | CGGTAAACTGCCCACTTGGCAGTACATCAAGTGTATCATATGCCAAGTA | |
| | CGCCCCCTATTGACGTCAATGACGGTAAATGGCCCGCCTGGCATTATG | |
| | CCCAGTACATGACCTTATGGGACTTTCCTACTTGGCAGTACATCTACGT | |
| | ATTAGTCATCGCTATTACCATGGTGATGCGGTTTTGGCAGTACATCAAT | |
| | GGGCGTGGATAGCGGTTTGACTCACGGGGATTTCCAAGTCTCCACCCC | |
| | ATTGACGTCAATGGGAGTTTGTTTTGGCACCAAAATCAACGGGACTTTC | |
| | CAAAATGTCGTAACAACTCCGCCCCATTGACGCAAATGGGCGGTAGGC | |
| | GTGTACGGTGGGAGGTCTATATAAGCAGAGCTGGTTTAGTGAACCGTC | |
| | AGATCGAGAATAAACTAGTATTCTTCTGGTCCCCACAGACTCAGAGAGA | |
| | ACCCGCCACCATGAGCGGCTTCAGGAAGATGGCCTTCCCCAGCGGCA | |
| | AGGTGGAGGGCTGCATGGTGCAGGTGACCTGCGGCACCACCACCCTG | |
| | AACGGCCTGTGGCTGGACGACGTGGTGTACTGCCCCAGGCACGTGAT | |
| | CTGCACCAGCGAGGACATGCTGAACCCCAACTACGAGGACCTGCTGAT | |
| | CAGGAAGAGCAACCACAACTTCCTGGTGCAGGCCGGCAACGTGCAGCT | |
| | GAGGGTGATCGGCCACAGCATGCAGAACTGCGTGCTGAAGCTGAAGG | |
| | TGGACACCGCCAACCCCAAGACCCCCAAGTACAAGTTCGTGAGGATCC | |
| | AGCCCGGCCAGACCTTCAGCGTGCTGGCCTGCTACAACGGCAGCCCC | |
| | AGCGGCGTGTACCAGTGCGCCATGAGGCCCAACTTCACCATCAAGGGC | |
| | AGCTTCCTGAACGGCAGCTGCGGCAGCGTGGGCTTCAACATCGACTAC | |
| | GACTGCGTGAGCTTCTGCTACATGCACCACATGGAGCTGCCCACCGGC | |
| | GTGCACGCCGGCACCGACCTGGAGGGCAACTTCTACGGCCCCTTCGT | |
| | GGACAGGCAGACCGCCCAGGCCGCCGGCACCGACACCACCATCACCG | |
| | TGAACGTGCTGGCCTGGCTGTACGCCGCCGTGATCAACGGCGACAGG | |
| | TGGTTCCTGAACAGGTTCACCACCACCCTGAACGACTTCAACCTGGTG | |
| | GCCATGAAGTACAACTACGAGCCCCTGACCCAGGACCACGTGGACATC | |
| | CTGGGCCCCCTGAGCGCCCAGACCGGCATCGCCGTGCTGGACATGTG | |
| | CGCCAGCCTGAAGGAGCTGCTGCAGAACGGCATGAACGGCAGGACCA | |
| | TCCTGGGCAGCGCCCTGCTGGAGGACGAGTTCACCCCCTTCGACGTG | |
| | GTGAGGCAGTGCAGCGGCGTGACCTTCCAGTGATGACTCGAGCTGGTA | |
| | CTGCATGCACGCAATGCTAGCTGCCCCTTTCCCGTCCTGGGTACCCCG | |
| | AGTCTCCCCCGACCTCGGGTCCCAGGTATGCTCCCACCTCCACCTGCC | |
| | CCACTCACCACCTCTGCTAGTTCCAGACACCTCCCAAGCACGCAGCAA | |
| | TGCAGCTCAAAACGCTTAGCCTAGCCACACCCCCACGGGAAACAGCAG | |
| | TGATTAACCTTTAGCAATAAACGAAAGTTTAACTAAGCTATACTAACCCC | |
| | AGGGTTGGTCAATTTCGTGCCAGCCACACCCTGGAGCTAGCAAAAAAA | |
| | A | |
| 45 | TAGTTATTAATAGTAATCAATTACGGGGTCATTAGTTCATAGCCCATATA | 330 |
| Pan-CoV | TGGAGTTCCGCGTTACATAACTTACGGTAAATGGCCCGCCTGGCTGAC | |
| rAdv5-CMV- | CGCCCAACGACCCCCGCCCATTGACGTCAATAATGACGTATGTTCCCAT | |
| NSP12 | AGTAACGCCAATAGGGACTTTCCATTGACGTCAATGGGTGGAGTATTTA | |
| | CGGTAAACTGCCCACTTGGCAGTACATCAAGTGTATCATATGCCAAGTA | |
| | CGCCCCCTATTGACGTCAATGACGGTAAATGGCCCGCCTGGCATTATG | |
| | CCCAGTACATGACCTTATGGGACTTTCCTACTTGGCAGTACATCTACGT | |
| | ATTAGTCATCGCTATTACCATGGTGATGCGGTTTTGGCAGTACATCAAT | |
| | GGGCGTGGATAGCGGTTTGACTCACGGGGATTTCCAAGTCTCCACCCC | |
| | ATTGACGTCAATGGGAGTTTGTTTTGGCACCAAAATCAACGGGACTTTC | |
| | CAAAATGTCGTAACAACTCCGCCCCATTGACGCAAATGGGCGGTAGGC | |
| | GTGTACGGTGGGAGGTCTATATAAGCAGAGCTGGTTTAGTGAACCGTC | |
| | AGATCGAGAATAAACTAGTATTCTTCTGGTCCCCACAGACTCAGAGAGA | |
| | ACCCGCCACCATGAGCGCCGACGCCCAGAGCTTCCTGAACAGGGTGT | |
| | GCGGCGTGAGCGCCGCCAGGCTGACCCCCTGCGGCACCGGCACCAG | |
| | CACCGACGTGGTGTACAGGGCCTTCGACATCTACAACGACAAGGTGGC | |
| | CGGCTTCGCCAAGTTCCTGAAGACCAACTGCTGCAGGTTCCAGGAGAA | |
| | GGACGAGGACGACAACCTGATCGACAGCTACTTCGTGGTGAAGAGGCA | |
| | CACCTTCAGCAACTACCAGCACGAGGAGACCATCTACAACCTGCTGAA | |
| | GGACTGCCCCGCCGTGGCCAAGCACGACTTCTTCAAGTTCAGGATCGA | |
| | CGGCGACATGGTGCCCCACATCAGCAGGCAGAGGCTGACCAAGTACA | |
| | CCATGGCCGACCTGGTGTACGCCCTGAGGCACTTCGACGAGGGCAAC | |
| | TGCGACACCCTGAAGGAGATCCTGGTGACCTACAACTGCTGCGACGAC | |
| | GACTACTTCAACAAGAAGGACTGGTACGACTTCGTGGAGAACCCCGAC | |
| | ATCCTGAGGGTGTACGCCAACCTGGGCGAGAGGGTGAGGCAGGCCCT | |
| | GCTGAAGACCGTGCAGTTCTGCGACGCCATGAGGAACGCCGGCATCG | |
| | TGGGCGTGCTGACCCTGGACAACCAGGACCTGAACGGCAACTGGTAC | |
| | GACTTCGGCGACTTCATCCAGACCACCCCCGGCAGCGGCGTGCCCGT | |
| | GGTGGACAGCTACTACAGCCTGCTGATGCCCATCCTGACCCTGACCAG | |
| | GGCCCTGACCGCCGAGAGCCACGTGGACACCGACCTGACCAAGCCCT | |
| | ACATCAAGTGGGACCTGCTGAAGTACGACTTCACCGAGGAGAGGCTGA | |
| | AGCTGTTCGACAGGTACTTCAAGTACTGGGACCAGACCTACCACCCCA | |
| | ACTGCGTGAACTGCCTGGACGACAGGTGCATCCTGCACTGCGCCAACT | |
| | TCAACGTGCTGTTCAGCACCGTGTTCCCCCCCACCAGCTTCGGCCCCC | |
| | TGGTGAGGAAGATCTTCGTGGACGGCGTGCCCTTCGTGGTGAGCACC | |
| | GGCTACCACTTCAGGGAGCTGGGCGTGGTGCACAACCAGGACGTGAA | |
| | CCTGCACAGCAGCAGGCTGAGCTTCAAGGAGCTGCTGGTGTACGCCG | |
| | CCGACCCCGCCATGCACGCCGCCAGCGGCAACCTGCTGCTGGACAAG | |
| | AGGACCACCTGCTTCAGCGTGGCCGCCCTGACCAACAACGTGGCCTTC | |
| | CAGACCGTGAAGCCCGGCAACTTCAACAAGGACTTCTACGACTTCGCC | |
| | GTGAGCAAGGGCTTCTTCAAGGAGGGCAGCAGCGTGGAGCTGAAGCA | |
| | CTTCTTCTTCGCCCAGGACGGCAACGCCGCCATCAGCGACTACGACTA | |
| | CTACAGGTACAACCTGCCCACCATGTGCGACATCAGGCAGCTGCTGTT | |
| | CGTGGTGGAGGTGGTGGACAAGTACTTCGACTGCTACGACGGCGGCT | |
| | GCATCAACGCCAACCAGGTGATCGTGAACAACCTGGACAAGAGCGCCG | |
| | GCTTCCCCTTCAACAAGTGGGGCAAGGCCAGGCTGTACTACGACAGCA | |
| | TGAGCTACGAGGACCAGGACGCCCTGTTCGCCTACACCAAGAGGAACG | |
| | TGATCCCCACCATCACCCAGATGAACCTGAAGTACGCCATCAGCGCCA | |
| | AGAACAGGGCCAGGACCGTGGCCGGCGTGAGCATCTGCAGCACCATG | |
| | ACCAACAGGCAGTTCCACCAGAAGCTGCTGAAGAGCATCGCCGCCACC | |
| | AGGGGCGCCACCGTGGTGATCGGCACCAGCAAGTTCTACGGCGGCTG | |
| | GCACAACATGCTGAAGACCGTGTACAGCGACGTGGAGAACCCCCACCT | |
| | GATGGGCTGGGACTACCCCAAGTGCGACAGGGCCATGCCCAACATGC | |
| | TGAGGATCATGGCCAGCCTGGTGCTGGCCAGGAAGCACACCACCTGCT | |
| | GCAGCCTGAGCCACAGGTTCTACAGGCTGGCCAACGAGTGCGCCCAG | |
| | GTGCTGAGCGAGATGGTGATGTGCGGCGGCAGCCTGTACGTGAAGCC | |
| | CGGCGGCACCAGCAGCGGCGACGCCACCACCGCCTACGCCAACAGCG | |
| | TGTTCAACATCTGCCAGGCCGTGACCGCCAACGTGAACGCCCTGCTGA | |
| | GCACCGACGGCAACAAGATCGCCGACAAGTACGTGAGGAACCTGCAG | |
| | CACAGGCTGTACGAGTGCCTGTACAGGAACAGGGACGTGGACACCGA | |
| | CTTCGTGAACGAGTTCTACGCCTACCTGAGGAAGCACTTCAGCATGATG | |
| | ATCCTGAGCGACGACGCCGTGGTGTGCTTCAACAGCACCTACGCCAGC | |
| | CAGGGCCTGGTGGCCAGCATCAAGAACTTCAAGAGCGTGCTGTACTAC | |
| | CAGAACAACGTGTTCATGAGCGAGGCCAAGTGCTGGACCGAGACCGAC | |
| | CTGACCAAGGGCCCCCACGAGTTCTGCAGCCAGCACACCATGCTGGTG | |
| | AAGCAGGGCGACGACTACGTGTACCTGCCCTACCCCGACCCCAGCAG | |
| | GATCCTGGGCGCCGGCTGCTTCGTGGACGACATCGTGAAGACCGACG | |
| | GCACCCTGATGATCGAGAGGTTCGTGAGCCTGGCCATCGACGCCTACC | |
| | CCCTGACCAAGCACCCCAACCAGGAGTACGCCGACGTGTTCCACCTGT | |
| | ACCTGCAGTACATCAGGAAGCTGCACGACGAGCTGACCGGCCACATGC | |
| | TGGACATGTACAGCGTGATGCTGACCAACGACAACACCAGCAGGTACT | |
| | GGGAGCCCGAGTTCTACGAGGCCATGTACACCCCCCACACCGTGCTG | |
| | CAGTGATGACTCGAGCTGGTACTGCATGCACGCAATGCTAGCTGCCCC | |
| | TTTCCCGTCCTGGGTACCCCGAGTCTCCCCCGACCTCGGGTCCCAGGT | |
| | ATGCTCCCACCTCCACCTGCCCCACTCACCACCTCTGCTAGTTCCAGAC | |
| | ACCTCCCAAGCACGCAGCAATGCAGCTCAAAACGCTTAGCCTAGCCAC | |
| | ACCCCCACGGGAAACAGCAGTGATTAACCTTTAGCAATAAACGAAAGTT | |
| | TAACTAAGCTATACTAACCCCAGGGTTGGTCAATTTCGTGCCAGCCACA | |
| | CCCTGGAGCTAGCAAAAAAAA | |
| 46 | CTCGACATTGATTATTGACTAGTTATTAATAGTAATCAATTACGGGGTCA | 331 |
| Pan-CoV | TTAGTTCATAGCCCATATATGGAGTTCCGCGTTACATAACTTACGGTAAA | |
| rAdv5-CAG- | TGGCCCGCCTGGCTGACCGCCCAACGACCCCCGCCCATTGACGTCAAT | |
| Nucleocapsid | AATGACGTATGTTCCCATAGTAACGCCAATAGGGACTTTCCATTGACGT | |
| | CAATGGGTGGAGTATTTACGGTAAACTGCCCACTTGGCAGTACATCAAG | |
| | TGTATCATATGCCAAGTACGCCCCCTATTGACGTCAATGACGGTAAATG | |
| | GCCCGCCTGGCATTATGCCCAGTACATGACCTTATGGGACTTTCCTACT | |
| | TGGCAGTACATCTACGTATTAGTCATCGCTATTACCATGGTCGAGGTGA | |
| | GCCCCACGTTCTGCTTCACTCTCCCCATCTCCCCCCCCTCCCCACCCC | |
| | CAATTTTGTATTTATTTATTTTTTAATTATTTTGTGCAGCGATGGGGGCG | |
| | GGGGGGGGGGGGGGGCGCGCGCCAGGCGGGGCGGGGCGGGGCGA | |
| | GGGGCGGGGCGGGGCGAGGCGGAGAGGTGCGGCGGCAGCCAATCA | |
| | GAGCGGCGCGCTCCGAAAGTTTCCTTTTATGGCGAGGCGGCGGCGGC | |
| | GGCGGCCCTATAAAAAGCGAAGCGCGCGGCGGGCGGGAGTCGCTGC | |
| | GCGCTGCCTTCGCCCCGTGCCCCGCTCCGCCGCCGCCTCGCGCCGCC | |
| | CGCCCCGGCTCTGACTGACCGCGTTACTCCCACAGGTGAGCGGGCGG | |
| | GACGGCCCTTCTCCTCCGGGCTGTAATTAGCGCTTGGTTTAATGACGG | |
| | CTTGTTTCTTTTCTGTGGCTGCGTGAAAGCCTTGAGGGGCTCCGGGAG | |
| | GGCCCTTTGTGCGGGGGGAGCGGCTCGGGGGGTGCGTGCGTGTGTG | |
| | TGTGCGTGGGGAGCGCCGCGTGCGGCTCCGCGCTGCCCGGCGGCTG | |
| | TGAGCGCTGCGGGCGCGGCGCGGGGCTTTGTGCGCTCCGCAGTGTGC | |
| | GCGAGGGGAGCGCGGCCGGGGGCGGTGCCCCGCGGTGCGGGGGGG | |
| | GCTGCGAGGGGAACAAAGGCTGCGTGCGGGGTGTGTGCGTGGGGGG | |
| | GTGAGCAGGGGGTGTGGGCGCGTCGGTCGGGCTGCAACCCCCCCTG | |
| | CACCCCCCTCCCCGAGTTGCTGAGCACGGCCCGGCTTCGGGTGCGGG | |
| | GCTCCGTACGGGGCGTGGCGCGGGGCTCGCCGTGCCGGGCGGGGGG | |
| | TGGCGGCAGGTGGGGGTGCCGGGCGGGGCGGGGCCGCCTCGGGCC | |
| | GGGGAGGGCTCGGGGGAGGGGCGCGGCGGCCCCCGGAGCGCCGGC | |
| | GGCTGTCGAGGCGCGGCGAGCCGCAGCCATTGCCTTTTATGGTAATCG | |
| | TGCGAGAGGGCGCAGGGACTTCCTTTGTCCCAAATCTGTGCGGAGCCG | |
| | AAATCTGGGAGGCGCCGCCGCACCCCCTCTAGCGGGCGCGGGGCGAA | |
| | GCGGTGCGGCGCCGGCAGGAAGGAAATGGGCGGGGAGGGCCTTCGT | |
| | GCGTCGCCGCGCCGCCGTCCCCTTCTCCCTCTCCAGCCTCGGGGCTG | |
| | TCCGCGGGGGGACGGCTGCCTTCGGGGGGGACGGGGCAGGGCGGG | |
| | GTTCGGCTTCTGGCGTGTGACCGGCGGCTCTAGAGCCTCTGCTAACCA | |
| | TGTTCATGCCTTCTTCTTTTTCCTACAGCTCCTGGGCAACGTGCTGGTT | |
| | ATTGTGCTGTCTCATCATTTTGGCAAAGAATTGGAGAATAAACTAGTATT | |
| | CTTCTGGTCCCCACAGACTCAGAGAGAACCCGCCACCATGAGCGACAA | |
| | CGGCCCCCAGAACCAGAGGAACGCCCCCAGGATCACCTTCGGCGGCC | |
| | CCAGCGACAGCACCGGCAGCAACCAGAACGGCGAGAGGAGCGGCGC | |
| | CAGGAGCAAGCAGAGGAGGCCCCAGGGCCTGCCCAACAACACCGCCA | |
| | GCTGGTTCACCGCCCTGACCCAGCACGGCAAGGAGGACCTGAAGTTC | |
| | CCCAGGGGCCAGGGCGTGCCCATCAACACCAACAGCAGCCCCGACGA | |
| | CCAGATCGGCTACTACAGGAGGGCCACCAGGAGGATCAGGGGCGGCG | |
| | ACGGCAAGATGAAGGACCTGAGCCCCAGGTGGTACTTCTACTACCTGG | |
| | GCACCGGCCCCGAGGCCGGCCTGCCCTACGGCGCCAACAAGGACGG | |
| | CATCATCTGGGTGGCCACCGAGGGCGCCCTGAACACCCCCAAGGACC | |
| | ACATCGGCACCAGGAACCCCGCCAACAACGCCGCCATCGTGCTGCAG | |
| | CTGCCCCAGGGCACCACCCTGCCCAAGGGCTTCTACGCCGAGGGCAG | |
| | CAGGGGCGGCAGCCAGGCCAGCAGCAGGAGCAGCAGCAGGAGCAGG | |
| | AACAGCAGCAGGAACAGCACCCCCGGCAGCAGCAGGGGCACCAGCCC | |
| | CGCCAGGATGGCCGGCAACGGCGGCGACGCCGCCCTGGCCCTGCTG | |
| | CTGCTGGACAGGCTGAACCAGCTGGAGAGCAAGATGAGCGGCAAGGG | |
| | CCAGCAGCAGCAGGGCCAGACCGTGACCAAGAAGAGCGCCGCCGAGG | |
| | CCAGCAAGAAGCCCAGGCAGAAGAGGACCGCCACCAAGGCCTACAAC | |
| | GTGACCCAGGCCTTCGGCAGGAGGGGCCCCGAGCAGACCCAGGGCAA | |
| | CTTCGGCGACCAGGAGCTGATCAGGCAGGGCACCGACTACAAGCACT | |
| | GGCCCCAGATCGCCCAGTTCGCCCCCAGCGCCAGCGCCTTCTTCGGC | |
| | ATGAGCAGGATCGGCATGGAGGTGACCCCCAGCGGCACCTGGCTGAC | |
| | CTACACCGGCGCCATCAAGCTGGACGACAAGGACCCCAACTTCAAGGA | |
| | CCAGGTGATCCTGCTGAACAAGCACATCGACGCCTACAAGACCTTCCC | |
| | CCCCACCGAGCCCAAGAAGGACAAGAAGAAGAAGGCCGACGAGACCC | |
| | AGGCCCTGCCCCAGAGGCAGAAGAAGCAGCAGACCGTGACCCTGCTG | |
| | CCCGCCGCCGACCTGGACGACTTCAGCAAGCAGCTGCAGCAGAGCAT | |
| | GAGCAGCGCCGACAGCACCCAGGCCTGATGACTCGAGCTGGTACTGC | |
| | ATGCACGCAATGCTAGCTGCCCCTTTCCCGTCCTGGGTACCCCGAGTC | |
| | TCCCCCGACCTCGGGTCCCAGGTATGCTCCCACCTCCACCTGCCCCAC | |
| | TCACCACCTCTGCTAGTTCCAGACACCTCCCAAGCACGCAGCAATGCA | |
| | GCTCAAAACGCTTAGCCTAGCCACACCCCCACGGGAAACAGCAGTGAT | |
| | TAACCTTTAGCAATAAACGAAAGTTTAACTAAGCTATACTAACCCCAGGG | |
| | TTGGTCAATTTCGTGCCAGCCACACCCTGGAGCTAGCAAAAAAAA | |
| 47 | CTCGACATTGATTATTGACTAGTTATTAATAGTAATCAATTACGGGGTCA | 332 |
| Pan-CoV | TTAGTTCATAGCCCATATATGGAGTTCCGCGTTACATAACTTACGGTAAA | |
| rAdv5-CAG- | TGGCCCGCCTGGCTGACCGCCCAACGACCCCCGCCCATTGACGTCAAT | |
| ORF1a/b | AATGACGTATGTTCCCATAGTAACGCCAATAGGGACTTTCCATTGACGT | |
| | CAATGGGTGGAGTATTTACGGTAAACTGCCCACTTGGCAGTACATCAAG | |
| | TGTATCATATGCCAAGTACGCCCCCTATTGACGTCAATGACGGTAAATG | |
| | GCCCGCCTGGCATTATGCCCAGTACATGACCTTATGGGACTTTCCTACT | |
| | TGGCAGTACATCTACGTATTAGTCATCGCTATTACCATGGTCGAGGTGA | |
| | GCCCCACGTTCTGCTTCACTCTCCCCATCTCCCCCCCCTCCCCACCCC | |
| | CAATTTTGTATTTATTTATTTTTTAATTATTTTGTGCAGCGATGGGGGCG | |
| | GGGGGGGGGGGGGGGCGCGCGCCAGGCGGGGCGGGGCGGGGCGA | |
| | GGGGCGGGGCGGGGCGAGGCGGAGAGGTGCGGCGGCAGCCAATCA | |
| | GAGCGGCGCGCTCCGAAAGTTTCCTTTTATGGCGAGGCGGCGGCGGC | |
| | GGCGGCCCTATAAAAAGCGAAGCGCGCGGCGGGCGGGAGTCGCTGC | |
| | GCGCTGCCTTCGCCCCGTGCCCCGCTCCGCCGCCGCCTCGCGCCGCC | |
| | CGCCCCGGCTCTGACTGACCGCGTTACTCCCACAGGTGAGCGGGCGG | |
| | GACGGCCCTTCTCCTCCGGGCTGTAATTAGCGCTTGGTTTAATGACGG | |
| | CTTGTTTCTTTTCTGTGGCTGCGTGAAAGCCTTGAGGGGCTCCGGGAG | |
| | GGCCCTTTGTGCGGGGGGAGCGGCTCGGGGGGTGCGTGCGTGTGTG | |
| | TGTGCGTGGGGAGCGCCGCGTGCGGCTCCGCGCTGCCCGGCGGCTG | |
| | TGAGCGCTGCGGGCGCGGCGCGGGGCTTTGTGCGCTCCGCAGTGTGC | |
| | GCGAGGGGAGCGCGGCCGGGGGCGGTGCCCCGCGGTGCGGGGGGG | |
| | GCTGCGAGGGGAACAAAGGCTGCGTGCGGGGTGTGTGCGTGGGGGG | |
| | GTGAGCAGGGGGTGTGGGCGCGTCGGTCGGGCTGCAACCCCCCCTG | |
| | CACCCCCCTCCCCGAGTTGCTGAGCACGGCCCGGCTTCGGGTGCGGG | |
| | GCTCCGTACGGGGCGTGGCGCGGGGCTCGCCGTGCCGGGCGGGGGG | |
| | TGGCGGCAGGTGGGGGTGCCGGGCGGGGCGGGGCCGCCTCGGGCC | |
| | GGGGAGGGCTCGGGGGAGGGGCGCGGCGGCCCCCGGAGCGCCGGC | |
| | GGCTGTCGAGGCGCGGCGAGCCGCAGCCATTGCCTTTTATGGTAATCG | |
| | TGCGAGAGGGCGCAGGGACTTCCTTTGTCCCAAATCTGTGCGGAGCCG | |
| | AAATCTGGGAGGCGCCGCCGCACCCCCTCTAGCGGGCGCGGGGCGAA | |
| | GCGGTGCGGCGCCGGCAGGAAGGAAATGGGCGGGGAGGGCCTTCGT | |
| | GCGTCGCCGCGCCGCCGTCCCCTTCTCCCTCTCCAGCCTCGGGGCTG | |
| | TCCGCGGGGGGACGGCTGCCTTCGGGGGGGACGGGGCAGGGCGGG | |
| | GTTCGGCTTCTGGCGTGTGACCGGCGGCTCTAGAGCCTCTGCTAACCA | |
| | TGTTCATGCCTTCTTCTTTTTCCTACAGCTCCTGGGCAACGTGCTGGTT | |
| | ATTGTGCTGTCTCATCATTTTGGCAAAGAATTGGAGAATAAACTAGTATT | |
| | CTTCTGGTCCCCACAGACTCAGAGAGAACCCGCCACCATGGACCAGGA | |
| | ACCCCGCCTGGAGGAAGGCCGTGTTCATCAGCCCCTACAACAGCCAGA | |
| | ACGCCGTGGCCAGCAAGATCCTGGGCCTGCCCACCCAGACCGTGGAC | |
| | AGCAGCCAGGGCAGCGAGTACGACTACGTGATCTTCACCCAGACCACC | |
| | GAGACCGCCCACAGCTGCAACGTGAACAGGTTCAACGTGGCCATCACC | |
| | AGGGCCAAGGTGGGCATCCTGTGCATCATGAGCGACAGGGACCTGTA | |
| | CGACAAGCTGCAGTTCACCAGCCTGGAGATCCCCAGGAGGAACGTGG | |
| | CCACCCTGCAGGCCGAGAACGTGACCGGCCTGTTCAAGGACTGCAGC | |
| | AAGGTGATCACCGGCCTGCACCCCACCCAGGCCCCCACCCACCTGAG | |
| | CGTGGACACCAAGTTCAAGACCGAGGGCCTGTGCGTGGACATCCCCG | |
| | GCATCCCCAAGGACATGACCTACAGGAGGCTGATCAGCATGATGGGCT | |
| | TCAAGATGAACTACCAGGTGAACGGCTACCCCAACATGTTCATCACCAG | |
| | GGAGGAGGCCATCAGGCACGTGAGGGCCTGGATCGGCTTCGACGTGG | |
| | AGGGCTGCCACGCCACCAGGGAGGCCGTGGGCACCAACCTGCCCCTG | |
| | CAGCTGGGCTTCAGCACCGGCGTGAACCTGGTGGCCGTGCCCACCGG | |
| | CTACGTGGACACCCCCAACAACACCGACTTCAGCAGGGTGAGCGCCAA | |
| | GCCCCCCCCCGGCGACCAGTTCAAGCACCTGATCCCCCTGATGTACAA | |
| | GGGCCTGCCCTGGAACGTGGTGAGGATCAAGATCGTGCAGATGCTGA | |
| | GCGACACCCTGAAGAACCTGAGCGACAGGGTGGTGTTCGTGCTGTGG | |
| | GCCCACGGCTTCGAGCTGACCAGCATGAAGTACTTCGTGAAGATCGGC | |
| | CCCGAGAGGACCTGCTGCCTGTGCGACAGGAGGGCCACCTGCTTCAG | |
| | CACCGCCAGCGACACCTACGCCTGCTGGCACCACAGCATCGGCTTCGA | |
| | CTACGTGTACAACCCCTTCATGATCGACGTGCAGCAGTGGGGCTTCAC | |
| | CGGCAACCTGCAGAGCAACCACGACCTGTACTGCCAGGTGCACGGCA | |
| | ACGCCCACGTGGCCAGCTGCGACGCCATCATGACCAGGTGCCTGGCC | |
| | GTGCACGAGTGCTTCGTGAAGAGGGTGGACTGGACCATCGAGTACCCC | |
| | ATCATCGGCGACGAGCTGAAGATCAACGCCGCCTGCAGGAAGGTGCA | |
| | GCACATGGTGGTGAAGGCCGCCCTGCTGGCCGACAAGTTCCCCGTGC | |
| | TGCACGACATCGGCAACCCCAAGGCCATCAAGTGCGTGCCCCAGGCC | |
| | GACGTGGAGTGGAAGTTCTACGACGCCCAGCCCTGCAGCGACAAGGC | |
| | CTACAAGATCGAGGAGCTGTTCTACAGCTACGCCACCCACAGCGACAA | |
| | GTTCACCGACGGCGTGTGCCTGTTCTGGAACTGCAACGTGGACAGGTA | |
| | CCCCGCCAACAGCATCGTGTGCAGGTTCGACACCAGGGTGCTGAGCAA | |
| | CCTGAACCTGCCCGGCTGCGACGGCGGCAGCCTGTACGTGAACAAGC | |
| | ACGCCTTCCACACCCCCGCCTTCGACAAGAGCGCCTTCGTGAACCTGA | |
| | AGCAGCTGCCCTTCTTCTACTACAGCGACAGCCCCTGCGAGAGCCACG | |
| | GCAAGCAGGTGGTGAGCGACATCGACTACGTGCCCCTGAAGAGCGCC | |
| | ACCTGCATCACCAGGTGCAACCTGGGCGGCGCCGTGTGCAGGCACCA | |
| | CGCCAACGAGTACAGGCTGTACCTGGACGCCTACAACATGATGATCAG | |
| | CGCCGGCTTCAGCCTGTGGGTGTACAAGCAGTTCGACACCTACAACCT | |
| | GTGGAACACCTTCACCAGGCTGCAGAGCCTGGAGAACGTGGCCTTCAA | |
| | CGTGGTGAACAAGGGCCACTTCGACGGCCAGCAGGGCGAGGTGCCCG | |
| | TGAGCATCATCAACAACACCGTGTACACCAAGGTGGACGGCGTGGACG | |
| | TGGAGCTGTTCGAGAACAAGACCACCCTGCCCGTGAACGTGGCCTTCG | |
| | AGCTGTGGGCCAAGAGGAACATCAAGCCCGTGCCCGAGGTGAAGATC | |
| | CTGAACAACCTGGGCGTGGACATCGCCGCCAACACCGTGATCTGGGAC | |
| | TACAAGAGGGACGCCCCCGCCCACATCAGCACCATCGGCGTGTGCAG | |
| | CATGACCGACATCGCCAAGAAGCCCACCGAGACCATCTGCGCCCCCCT | |
| | GACCGTGTTCTTCGACGGCAGGGTGGACGGCCAGGTGGACCTGTTCA | |
| | GGAACGCCAGGAACGGCGTGCTGATCACCGAGGGCAGCGTGAAGGGC | |
| | CTGCAGCCCAGCGTGGGCCCCAAGCAGGCCAGCCTGAACGGCGTGAC | |
| | CCTGATCGGCGAGGCCGTGAAGACCCAGTTCAACTACTACAAGAAGGT | |
| | GGACGGCGTGGTGCAGCAGCTGCCCGAGACCTACTTCACCCAGAGCA | |
| | GGAACCTGCAGGAGTTCAAGCCCAGGAGCCAGATGGAGATCGACTTCC | |
| | TGGAGCTGGCCATGGACGAGTTCATCGAGAGGTACAAGCTGGAGGGC | |
| | TACGCCTTCGAGCACATCGTGTACGGCGACTTCAGCCACAGCCAGCTG | |
| | GGCGGCCTGCACCTGCTGATCGGCCTGGCCAAGAGGTTCAAGGAGAG | |
| | CCCCTTCGAGCTGGAGGACTTCATCCCCATGGACAGCACCGTGAAGAA | |
| | CTACTTCATCACCGACGCCCAGACCGGCAGCAGCAAGTGCGTGTGCAG | |
| | CGTGATCGACCTGCTGCTGGACGACTTCGTGGAGATCATCAAGAGCCA | |
| | GGACCTGAGCGTGGTGAGCAAGGTGGTGAAGGTGACCATCGACTACA | |
| | CCGAGATCAGCTTCATGCTGTGGTGCAAGGACGGCCACGTGGAGACCT | |
| | TCTACCCCAAGCTGCAGAGCAGCCAGGCCTGGCAGCCCGGCGTGGCC | |
| | ATGCCCAACCTGTACAAGATGCAGAGGATGCTGCTGGAGAAGTGCGAC | |
| | CTGCAGAACTACGGCGACAGCGCCACCCTGCCCAAGGGCATCATGATG | |
| | AACGTGGCCAAGTACACCCAGCTGTGCCAGTACCTGAACACCCTGACC | |
| | CTGGCCGTGCCCTACAACATGAGGGTGATCCACTTCGGCGCCGGCAG | |
| | CGACAAGGGCGTGGCCCCCGGCACCGCCGTGCTGAGGCAGTGGCTG | |
| | CCCACCGGCACCCTGCTGGTGGACAGCGACCTGAACGACTTCGTGAG | |
| | CGACGCCGACAGCACCCTGATCGGCGACTGCGCCACCGTGCACACCG | |
| | CCAACAAGTGGGACCTGATCATCAGCGACATGTACGACCCCAAGACCA | |
| | AGAACGTGACCAAGGAGAACGACAGCAAGGAGGGCTTCTTCACCTACA | |
| | TCTGCGGCTTCATCCAGCAGAAGCTGGCCCTGGGCGGCAGCGTGGCC | |
| | ATCAAGATCACCGAGCACAGCTGGAACGCCGACCTGTACAAGCTGATG | |
| | GGCCACTTCGCCTGGTGGACCGCCTTCGTGACCAACGTGAACGCCAG | |
| | CAGCAGCGAGGCCTTCCTGATCGGCTGCAACTACCTGGGCAAGCCCA | |
| | GGGAGCAGATCGACGGCTACGTGATGCACGCCAACTACATCTTCTGGA | |
| | GGAACACCAACCCCATCCAGCTGAGCAGCTACAGCCTGTTCGACATGA | |
| | GCAAGTTCCCCCTGAAGCTTGATGACTCGAGCTGGTACTGCATGCACG | |
| | CAATGCTAGCTGCCCCTTTCCCGTCCTGGGTACCCCGAGTCTCCCCCG | |
| | ACCTCGGGTCCCAGGTATGCTCCCACCTCCACCTGCCCCACTCACCAC | |
| | CTCTGCTAGTTCCAGACACCTCCCAAGCACGCAGCAATGCAGCTCAAA | |
| | ACGCTTAGCCTAGCCACACCCCCACGGGAAACAGCAGTGATTAACCTT | |
| | TAGCAATAAACGAAAGTTTAACTAAGCTATACTAACCCCAGGGTTGGTC | |
| | AATTTCGTGCCAGCCACACCCTGGAGCTAGCAAAAAAAA | |
| 48 | CTCGACATTGATTATTGACTAGTTATTAATAGTAATCAATTACGGGGTCA | 333 |
| Pan-CoV | TTAGTTCATAGCCCATATATGGAGTTCCGCGTTACATAACTTACGGTAAA | |
| rAdv5-CAG- | TGGCCCGCCTGGCTGACCGCCCAACGACCCCCGCCCATTGACGTCAAT | |
| Envelope | AATGACGTATGTTCCCATAGTAACGCCAATAGGGACTTTCCATTGACGT | |
| | CAATGGGTGGAGTATTTACGGTAAACTGCCCACTTGGCAGTACATCAAG | |
| | TGTATCATATGCCAAGTACGCCCCCTATTGACGTCAATGACGGTAAATG | |
| | GCCCGCCTGGCATTATGCCCAGTACATGACCTTATGGGACTTTCCTACT | |
| | TGGCAGTACATCTACGTATTAGTCATCGCTATTACCATGGTCGAGGTGA | |
| | GCCCCACGTTCTGCTTCACTCTCCCCATCTCCCCCCCCTCCCCACCCC | |
| | CAATTTTGTATTTATTTATTTTTTAATTATTTTGTGCAGCGATGGGGGCG | |
| | GGGGGGGGGGGGGGGCGCGCGCCAGGCGGGGCGGGGCGGGGCGA | |
| | GGGGCGGGGCGGGGCGAGGCGGAGAGGTGCGGCGGCAGCCAATCA | |
| | GAGCGGCGCGCTCCGAAAGTTTCCTTTTATGGCGAGGCGGCGGCGGC | |
| | GGCGGCCCTATAAAAAGCGAAGCGCGCGGCGGGCGGGAGTCGCTGC | |
| | GCGCTGCCTTCGCCCCGTGCCCCGCTCCGCCGCCGCCTCGCGCCGCC | |
| | CGCCCCGGCTCTGACTGACCGCGTTACTCCCACAGGTGAGCGGGCGG | |
| | GACGGCCCTTCTCCTCCGGGCTGTAATTAGCGCTTGGTTTAATGACGG | |
| | CTTGTTTCTTTTCTGTGGCTGCGTGAAAGCCTTGAGGGGCTCCGGGAG | |
| | GGCCCTTTGTGCGGGGGGAGCGGCTCGGGGGGTGCGTGCGTGTGTG | |
| | TGTGCGTGGGGAGCGCCGCGTGCGGCTCCGCGCTGCCCGGCGGCTG | |
| | TGAGCGCTGCGGGCGCGGCGCGGGGCTTTGTGCGCTCCGCAGTGTGC | |
| | GCGAGGGGAGCGCGGCCGGGGGCGGTGCCCCGCGGTGCGGGGGGG | |
| | GCTGCGAGGGGAACAAAGGCTGCGTGCGGGGTGTGTGCGTGGGGGG | |
| | GTGAGCAGGGGGTGTGGGCGCGTCGGTCGGGCTGCAACCCCCCCTG | |
| | CACCCCCCTCCCCGAGTTGCTGAGCACGGCCCGGCTTCGGGTGCGGG | |
| | GCTCCGTACGGGGCGTGGCGCGGGGCTCGCCGTGCCGGGCGGGGGG | |
| | TGGCGGCAGGTGGGGGTGCCGGGCGGGGCGGGGCCGCCTCGGGCC | |
| | GGGGAGGGCTCGGGGGAGGGGCGCGGCGGCCCCCGGAGCGCCGGC | |
| | GGCTGTCGAGGCGCGGCGAGCCGCAGCCATTGCCTTTTATGGTAATCG | |
| | TGCGAGAGGGCGCAGGGACTTCCTTTGTCCCAAATCTGTGCGGAGCCG | |
| | AAATCTGGGAGGCGCCGCCGCACCCCCTCTAGCGGGCGCGGGGCGAA | |
| | GCGGTGCGGCGCCGGCAGGAAGGAAATGGGCGGGGAGGGCCTTCGT | |
| | GCGTCGCCGCGCCGCCGTCCCCTTCTCCCTCTCCAGCCTCGGGGCTG | |
| | TCCGCGGGGGGACGGCTGCCTTCGGGGGGGACGGGGCAGGGCGGG | |
| | GTTCGGCTTCTGGCGTGTGACCGGCGGCTCTAGAGCCTCTGCTAACCA | |
| | TGTTCATGCCTTCTTCTTTTTCCTACAGCTCCTGGGCAACGTGCTGGTT | |
| | ATTGTGCTGTCTCATCATTTTGGCAAAGAATTGGAGAATAAACTAGTATT | |
| | CTTCTGGTCCCCACAGACTCAGAGAGAACCCGCCACCATGTACAGCTT | |
| | CGTGAGCGAGGAGACCGGCACCCTGATCGTGAACAGCGTGCTGCTGT | |
| | TCCTGGCCTTCGTGGTGTTCCTGCTGGTGACCCTGGCCATCCTGACCG | |
| | CCCTGAGGCTGTGCGCCTACTGCTGCAACATCGTGAACGTGAGCCTGG | |
| | TGAAGCCCAGCTTCTACGTGTACAGCAGGGTGAAGAACCTGAACAGCA | |
| | GCAGGGTGCCCGACCTGCTGGTGTGATGACTCGAGCTGGTACTGCATG | |
| | CACGCAATGCTAGCTGCCCCTTTCCCGTCCTGGGTACCCCGAGTCTCC | |
| | CCCGACCTCGGGTCCCAGGTATGCTCCCACCTCCACCTGCCCCACTCA | |
| | CCACCTCTGCTAGTTCCAGACACCTCCCAAGCACGCAGCAATGCAGCT | |
| | CAAAACGCTTAGCCTAGCCACACCCCCACGGGAAACAGCAGTGATTAA | |
| | CCTTTAGCAATAAACGAAAGTTTAACTAAGCTATACTAACCCCAGGGTT | |
| | GGTCAATTTCGTGCCAGCCACACCCTGGAGCTAGCAAAAAAAA | |
| 49 | CTCGACATTGATTATTGACTAGTTATTAATAGTAATCAATTACGGGGTCA | 334 |
| Pan-CoV | TTAGTTCATAGCCCATATATGGAGTTCCGCGTTACATAACTTACGGTAAA | |
| rAdv5-CAG- | TGGCCCGCCTGGCTGACCGCCCAACGACCCCCGCCCATTGACGTCAAT | |
| Membrane | AATGACGTATGTTCCCATAGTAACGCCAATAGGGACTTTCCATTGACGT | |
| | CAATGGGTGGAGTATTTACGGTAAACTGCCCACTTGGCAGTACATCAAG | |
| | TGTATCATATGCCAAGTACGCCCCCTATTGACGTCAATGACGGTAAATG | |
| | GCCCGCCTGGCATTATGCCCAGTACATGACCTTATGGGACTTTCCTACT | |
| | TGGCAGTACATCTACGTATTAGTCATCGCTATTACCATGGTCGAGGTGA | |
| | GCCCCACGTTCTGCTTCACTCTCCCCATCTCCCCCCCCTCCCCACCCC | |
| | CAATTTTGTATTTATTTATTTTTTAATTATTTTGTGCAGCGATGGGGGCG | |
| | GGGGGGGGGGGGGGGCGCGCGCCAGGCGGGGCGGGGCGGGGCGA | |
| | GGGGCGGGGCGGGGCGAGGCGGAGAGGTGCGGCGGCAGCCAATCA | |
| | GAGCGGCGCGCTCCGAAAGTTTCCTTTTATGGCGAGGCGGCGGCGGC | |
| | GGCGGCCCTATAAAAAGCGAAGCGCGCGGCGGGCGGGAGTCGCTGC | |
| | GCGCTGCCTTCGCCCCGTGCCCCGCTCCGCCGCCGCCTCGCGCCGCC | |
| | CGCCCCGGCTCTGACTGACCGCGTTACTCCCACAGGTGAGCGGGCGG | |
| | GACGGCCCTTCTCCTCCGGGCTGTAATTAGCGCTTGGTTTAATGACGG | |
| | CTTGTTTCTTTTCTGTGGCTGCGTGAAAGCCTTGAGGGGCTCCGGGAG | |
| | GGCCCTTTGTGCGGGGGGAGCGGCTCGGGGGGTGCGTGCGTGTGTG | |
| | TGTGCGTGGGGAGCGCCGCGTGCGGCTCCGCGCTGCCCGGCGGCTG | |
| | TGAGCGCTGCGGGCGCGGCGCGGGGCTTTGTGCGCTCCGCAGTGTGC | |
| | GCGAGGGGAGCGCGGCCGGGGGCGGTGCCCCGCGGTGCGGGGGGG | |
| | GCTGCGAGGGGAACAAAGGCTGCGTGCGGGGTGTGTGCGTGGGGGG | |
| | GTGAGCAGGGGGTGTGGGCGCGTCGGTCGGGCTGCAACCCCCCCTG | |
| | CACCCCCCTCCCCGAGTTGCTGAGCACGGCCCGGCTTCGGGTGCGGG | |
| | GCTCCGTACGGGGCGTGGCGCGGGGCTCGCCGTGCCGGGCGGGGGG | |
| | TGGCGGCAGGTGGGGGTGCCGGGCGGGGCGGGGCCGCCTCGGGCC | |
| | GGGGAGGGCTCGGGGGAGGGGCGCGGCGGCCCCCGGAGCGCCGGC | |
| | GGCTGTCGAGGCGCGGCGAGCCGCAGCCATTGCCTTTTATGGTAATCG | |
| | TGCGAGAGGGCGCAGGGACTTCCTTTGTCCCAAATCTGTGCGGAGCCG | |
| | AAATCTGGGAGGCGCCGCCGCACCCCCTCTAGCGGGCGCGGGGCGAA | |
| | GCGGTGCGGCGCCGGCAGGAAGGAAATGGGCGGGGAGGGCCTTCGT | |
| | GCGTCGCCGCGCCGCCGTCCCCTTCTCCCTCTCCAGCCTCGGGGCTG | |
| | TCCGCGGGGGGACGGCTGCCTTCGGGGGGGACGGGGCAGGGCGGG | |
| | GTTCGGCTTCTGGCGTGTGACCGGCGGCTCTAGAGCCTCTGCTAACCA | |
| | TGTTCATGCCTTCTTCTTTTTCCTACAGCTCCTGGGCAACGTGCTGGTT | |
| | ATTGTGCTGTCTCATCATTTTGGCAAAGAATTGGAGAATAAACTAGTATT | |
| | CTTCTGGTCCCCACAGACTCAGAGAGAACCCGCCACCATGGCCGACAG | |
| | CAACGGCACCATCACCGTGGAGGAGCTGAAGAAGCTGCTGGAGCAGT | |
| | GGAACCTGGTGATCGGCTTCCTGTTCCTGACCTGGATCTGCCTGCTGC | |
| | AGTTCGCCTACGCCAACAGGAACAGGTTCCTGTACATCATCAAGCTGAT | |
| | CTTCCTGTGGCTGCTGTGGCCCGTGACCCTGGCCTGCTTCGTGCTGGC | |
| | CGCCGTGTACAGGATCAACTGGATCACCGGCGGCATCGCCATCGCCAT | |
| | GGCCTGCCTGGTGGGCCTGATGTGGCTGAGCTACTTCATCGCCAGCTT | |
| | CAGGCTGTTCGCCAGGACCAGGAGCATGTGGAGCTTCAACCCCGAGA | |
| | CCAACATCCTGCTGAACGTGCCCCTGCACGGCACCATCCTGACCAGGC | |
| | CCCTGCTGGAGAGCGAGCTGGTGATCGGCGCCGTGATCCTGAGGGGC | |
| | CACCTGAGGATCGCCGGCCACCACCTGGGCAGGTGCGACATCAAGGA | |
| | CCTGCCCAAGGAGATCACCGTGGCCACCAGCAGGACCCTGAGCTACTA | |
| | CAAGCTGGGCGCCAGCCAGAGGGTGGCCGGCGACAGCGGCTTCGCC | |
| | GCCTACAGCAGGTACAGGATCGGCAACTACAAGCTGAACACCGACCAC | |
| | AGCAGCAGCAGCGACAACATCGCCCTGCTGGTGCAGTGATGACTCGAG | |
| | CTGGTACTGCATGCACGCAATGCTAGCTGCCCCTTTCCCGTCCTGGGT | |
| | ACCCCGAGTCTCCCCCGACCTCGGGTCCCAGGTATGCTCCCACCTCCA | |
| | CCTGCCCCACTCACCACCTCTGCTAGTTCCAGACACCTCCCAAGCACG | |
| | CAGCAATGCAGCTCAAAACGCTTAGCCTAGCCACACCCCCACGGGAAA | |
| | CAGCAGTGATTAACCTTTAGCAATAAACGAAAGTTTAACTAAGCTATACT | |
| | AACCCCAGGGTTGGTCAATTTCGTGCCAGCCACACCCTGGAGCTAGCA | |
| | AAAAAAA | |
| 50 | CTCGACATTGATTATTGACTAGTTATTAATAGTAATCAATTACGGGGTCA | 335 |
| Pan-CoV | TTAGTTCATAGCCCATATATGGAGTTCCGCGTTACATAACTTACGGTAAA | |
| rAdv5-CAG- | TGGCCCGCCTGGCTGACCGCCCAACGACCCCCGCCCATTGACGTCAAT | |
| ORF7a | AATGACGTATGTTCCCATAGTAACGCCAATAGGGACTTTCCATTGACGT | |
| | CAATGGGTGGAGTATTTACGGTAAACTGCCCACTTGGCAGTACATCAAG | |
| | TGTATCATATGCCAAGTACGCCCCCTATTGACGTCAATGACGGTAAATG | |
| | GCCCGCCTGGCATTATGCCCAGTACATGACCTTATGGGACTTTCCTACT | |
| | TGGCAGTACATCTACGTATTAGTCATCGCTATTACCATGGTCGAGGTGA | |
| | GCCCCACGTTCTGCTTCACTCTCCCCATCTCCCCCCCCTCCCCACCCC | |
| | CAATTTTGTATTTATTTATTTTTTAATTATTTTGTGCAGCGATGGGGGCG | |
| | GGGGGGGGGGGGGGGCGCGCGCCAGGCGGGGCGGGGCGGGGCGA | |
| | GGGGCGGGGCGGGGCGAGGCGGAGAGGTGCGGCGGCAGCCAATCA | |
| | GAGCGGCGCGCTCCGAAAGTTTCCTTTTATGGCGAGGCGGCGGCGGC | |
| | GGCGGCCCTATAAAAAGCGAAGCGCGCGGCGGGCGGGAGTCGCTGC | |
| | GCGCTGCCTTCGCCCCGTGCCCCGCTCCGCCGCCGCCTCGCGCCGCC | |
| | CGCCCCGGCTCTGACTGACCGCGTTACTCCCACAGGTGAGCGGGCGG | |
| | GACGGCCCTTCTCCTCCGGGCTGTAATTAGCGCTTGGTTTAATGACGG | |
| | CTTGTTTCTTTTCTGTGGCTGCGTGAAAGCCTTGAGGGGCTCCGGGAG | |
| | GGCCCTTTGTGCGGGGGGAGCGGCTCGGGGGGTGCGTGCGTGTGTG | |
| | TGTGCGTGGGGAGCGCCGCGTGCGGCTCCGCGCTGCCCGGCGGCTG | |
| | TGAGCGCTGCGGGCGCGGCGCGGGGCTTTGTGCGCTCCGCAGTGTGC | |
| | GCGAGGGGAGCGCGGCCGGGGGCGGTGCCCCGCGGTGCGGGGGGG | |
| | GCTGCGAGGGGAACAAAGGCTGCGTGCGGGGTGTGTGCGTGGGGGG | |
| | GTGAGCAGGGGGTGTGGGCGCGTCGGTCGGGCTGCAACCCCCCCTG | |
| | CACCCCCCTCCCCGAGTTGCTGAGCACGGCCCGGCTTCGGGTGCGGG | |
| | GCTCCGTACGGGGCGTGGCGCGGGGCTCGCCGTGCCGGGCGGGGGG | |
| | TGGCGGCAGGTGGGGGTGCCGGGCGGGGCGGGGCCGCCTCGGGCC | |
| | TGGCGGCAGGTGGGGGTGCCGGGCGGGGCGGGGCCGCCTCGGGCC | |
| | GGGGAGGGCTCGGGGGAGGGGCGCGGCGGCCCCCGGAGCGCCGGC | |
| | GGCTGTCGAGGCGCGGCGAGCCGCAGCCATTGCCTTTTATGGTAATCG | |
| | TGCGAGAGGGCGCAGGGACTTCCTTTGTCCCAAATCTGTGCGGAGCCG | |
| | AAATCTGGGAGGCGCCGCCGCACCCCCTCTAGCGGGCGCGGGGCGAA | |
| | GCGGTGCGGCGCCGGCAGGAAGGAAATGGGCGGGGAGGGCCTTCGT | |
| | GCGTCGCCGCGCCGCCGTCCCCTTCTCCCTCTCCAGCCTCGGGGCTG | |
| | TCCGCGGGGGGACGGCTGCCTTCGGGGGGGACGGGGCAGGGCGGG | |
| | GTTCGGCTTCTGGCGTGTGACCGGCGGCTCTAGAGCCTCTGCTAACCA | |
| | TGTTCATGCCTTCTTCTTTTTCCTACAGCTCCTGGGCAACGTGCTGGTT | |
| | ATTGTGCTGTCTCATCATTTTGGCAAAGAATTGGAGAATAAACTAGTATT | |
| | CTTCTGGTCCCCACAGACTCAGAGAGAACCCGCCACCATGAAGATCAT | |
| | CCTGTTCCTGGCCCTGATCACCCTGGCCACCTGCGAGCTGTACCACTA | |
| | CCAGGAGTGCGTGAGGGGCACCACCGTGTGATGACTCGAGCTGGTAC | |
| | TGCATGCACGCAATGCTAGCTGCCCCTTTCCCGTCCTGGGTACCCCGA | |
| | GTCTCCCCCGACCTCGGGTCCCAGGTATGCTCCCACCTCCACCTGCCC | |
| | CACTCACCACCTCTGCTAGTTCCAGACACCTCCCAAGCACGCAGCAAT | |
| | GCAGCTCAAAACGCTTAGCCTAGCCACACCCCCACGGGAAACAGCAGT | |
| | GATTAACCTTTAGCAATAAACGAAAGTTTAACTAAGCTATACTAACCCCA | |
| | GGGTTGGTCAATTTCGTGCCAGCCACACCCTGGAGCTAGCAAAAAAAA | |
| 51 | CTCGACATTGATTATTGACTAGTTATTAATAGTAATCAATTACGGGGTCA | 336 |
| Pan-CoV | TTAGTTCATAGCCCATATATGGAGTTCCGCGTTACATAACTTACGGTAAA | |
| rAdv-CAG- | TGGCCCGCCTGGCTGACCGCCCAACGACCCCCGCCCATTGACGTCAAT | |
| NSP3 | AATGACGTATGTTCCCATAGTAACGCCAATAGGGACTTTCCATTGACGT | |
| | CAATGGGTGGAGTATTTACGGTAAACTGCCCACTTGGCAGTACATCAAG | |
| | TGTATCATATGCCAAGTACGCCCCCTATTGACGTCAATGACGGTAAATG | |
| | GCCCGCCTGGCATTATGCCCAGTACATGACCTTATGGGACTTTCCTACT | |
| | TGGCAGTACATCTACGTATTAGTCATCGCTATTACCATGGTCGAGGTGA | |
| | GCCCCACGTTCTGCTTCACTCTCCCCATCTCCCCCCCCTCCCCACCCC | |
| | CAATTTTGTATTTATTTATTTTTTAATTATTTTGTGCAGCGATGGGGGCG | |
| | GGGGGGGGGGGGGGGCGCGCGCCAGGCGGGGCGGGGCGGGGCGA | |
| | GGGGCGGGGCGGGGCGAGGCGGAGAGGTGCGGCGGCAGCCAATCA | |
| | GAGCGGCGCGCTCCGAAAGTTTCCTTTTATGGCGAGGCGGCGGCGGC | |
| | GGCGGCCCTATAAAAAGCGAAGCGCGCGGCGGGCGGGAGTCGCTGC | |
| | GCGCTGCCTTCGCCCCGTGCCCCGCTCCGCCGCCGCCTCGCGCCGCC | |
| | CGCCCCGGCTCTGACTGACCGCGTTACTCCCACAGGTGAGCGGGCGG | |
| | GACGGCCCTTCTCCTCCGGGCTGTAATTAGCGCTTGGTTTAATGACGG | |
| | CTTGTTTCTTTTCTGTGGCTGCGTGAAAGCCTTGAGGGGCTCCGGGAG | |
| | GGCCCTTTGTGCGGGGGGAGCGGCTCGGGGGGTGCGTGCGTGTGTG | |
| | TGTGCGTGGGGAGCGCCGCGTGCGGCTCCGCGCTGCCCGGCGGCTG | |
| | TGAGCGCTGCGGGCGCGGCGCGGGGCTTTGTGCGCTCCGCAGTGTGC | |
| | GCGAGGGGAGCGCGGCCGGGGGCGGTGCCCCGCGGTGCGGGGGGG | |
| | GCTGCGAGGGGAACAAAGGCTGCGTGCGGGGTGTGTGCGTGGGGGG | |
| | GTGAGCAGGGGGTGTGGGCGCGTCGGTCGGGCTGCAACCCCCCCTG | |
| | CACCCCCCTCCCCGAGTTGCTGAGCACGGCCCGGCTTCGGGTGCGGG | |
| | GCTCCGTACGGGGCGTGGCGCGGGGCTCGCCGTGCCGGGCGGGGGG | |
| | TGGCGGCAGGTGGGGGTGCCGGGCGGGGCGGGGCCGCCTCGGGCC | |
| | GGGGAGGGCTCGGGGGAGGGGCGCGGCGGCCCCCGGAGCGCCGGC | |
| | GGCTGTCGAGGCGCGGCGAGCCGCAGCCATTGCCTTTTATGGTAATCG | |
| | TGCGAGAGGGCGCAGGGACTTCCTTTGTCCCAAATCTGTGCGGAGCCG | |
| | AAATCTGGGAGGCGCCGCCGCACCCCCTCTAGCGGGCGCGGGGCGAA | |
| | GCGGTGCGGCGCCGGCAGGAAGGAAATGGGCGGGGAGGGCCTTCGT | |
| | GCGTCGCCGCGCCGCCGTCCCCTTCTCCCTCTCCAGCCTCGGGGCTG | |
| | TCCGCGGGGGGACGGCTGCCTTCGGGGGGGACGGGGCAGGGCGGG | |
| | GTTCGGCTTCTGGCGTGTGACCGGCGGCTCTAGAGCCTCTGCTAACCA | |
| | TGTTCATGCCTTCTTCTTTTTCCTACAGCTCCTGGGCAACGTGCTGGTT | |
| | ATTGTGCTGTCTCATCATTTTGGCAAAGAATTGGAGAATAAACTAGTATT | |
| | CTTCTGGTCCCCACAGACTCAGAGAGAACCCGCCACCATGGCCCCCAC | |
| | CAAGGTGACCTTCGGCGACGACACCGTGATCGAGGTGCAGGGCTACA | |
| | AGAGCGTGAACATCACCTTCGAGCTGGACGAGAGGATCGACAAGGTGC | |
| | TGAACGAGAAGTGCAGCGCCTACACCGTGGAGCTGGGCACCGAGGTG | |
| | AACGAGTTCGCCTGCGTGGTGGCCGACGCCGTGATCAAGACCCTGCA | |
| | GCCCGTGAGCGAGCTGCTGACCCCCCTGGGCATCGACCTGGACGAGT | |
| | GGAGCATGGCCACCTACTACCTGTTCGACGAGAGCGGCGAGTTCAAGC | |
| | TGGCCAGCCACATGTACTGCAGCTTCTACCCCCCCGACGAGGACGAGG | |
| | AGGAGGGCGACTGCGAGGAGGAGGAGTTCGAGCCCAGCACCCAGTAC | |
| | GAGTACGGCACCGAGGACGACTACCAGGGCAAGCCCCTGGAGTTCGG | |
| | CGCCACCAGCGCCGCCCTGCAGCCCGAGGAGGAGCAGGAGGAGGAC | |
| | TGGCTGGACGACGACAGCCAGCAGACCGTGGGCCAGCAGGACGGCAG | |
| | CGAGGACAACCAGACCACCACCATCCAGACCATCGTGGAGGTGCAGC | |
| | CCCAGCTGGAGATGGAGCTGACCCCCGTGGTGCAGACCATCGAGGTG | |
| | AACAGCTTCAGCGGCTACCTGAAGCTGACCGACAACGTGTACATCAAG | |
| | AACGCCGACATCGTGGAGGAGGCCAAGAAGGTGAAGCCCACCGTGGT | |
| | GGTGAACGCCGCCAACGTGTACCTGAAGCACGGCGGCGGCGTGGCCG | |
| | GCGCCCTGAACAAGGCCACCAACAACGCCATGCAGGTGGAGAGCGAC | |
| | GACTACATCGCCACCAACGGCCCCCTGAAGGTGGGCGGCAGCTGCGT | |
| | GCTGAGCGGCCACAACCTGGCCAAGCACTGCCTGCACGTGGTGGGCC | |
| | CCAACGTGAACAAGGGCGAGGACATCCAGCTGCTGAAGAGCGCCTAC | |
| | GAGAACTTCAACCAGCACGAGGTGCTGCTGGCCCCCCTGCTGAGCGC | |
| | CGGCATCTTCGGCGCCGACCCCATCCACAGCCTGAGGGTGTGCGTGG | |
| | ACACCGTGAGGACCAACGTGTACCTGGCCGTGTTCGACAAGAACCTGT | |
| | ACGACAAGCTGGTGAGCAGCTTCCTGGAGATGAAGAGCGAGAAGCAG | |
| | GTGGAGCAGAAGATCGCCGAGATCCCCAAGGAGGAGGTGAAGCCCTT | |
| | CATCACCGAGAGCAAGCCCAGCGTGGAGCAGAGGAAGCAGGACGACA | |
| | AGAAGATCAAGGCCTGCGTGGAGGAGGTGACCACCACCCTGGAGGAG | |
| | ACCAAGTTCCTGACCGAGAACCTGCTGCTGTACATCGACATCAACGGC | |
| | AACCTGCACCCCGACAGCGCCACCCTGGTGAGCGACATCGACATCACC | |
| | TTCCTGAAGAAGGACGCCCCCTACATCGTGGGCGACGTGGTGCAGGA | |
| | GGGCGTGCTGACCGCCGTGGTGATCCCCACCAAGAAGGCCGGCGGCA | |
| | CCACCGAGATGCTGGCCAAGGCCCTGAGGAAGGTGCCCACCGACAAC | |
| | TACATCACCACCTACCCCGGCCAGGGCCTGAACGGCTACACCGTGGAG | |
| | GAGGCCAAGACCGTGCTGAAGAAGTGCAAGAGCGCCTTCTACATCCTG | |
| | CCCAGCATCATCAGCAACGAGAAGCAGGAGATCCTGGGCACCGTGAG | |
| | CTGGAACCTGAGGGAGATGCTGGCCCACGCCGAGGAGACCAGGAAGC | |
| | TGATGCCCGTGTGCGTGGAGACCAAGGCCATCGTGAGCACCATCCAGA | |
| | GGAAGTACAAGGGCATCAAGATCCAGGAGGGCGTGGTGGACTACGGC | |
| | GCCAGGTTCTACTTCTACACCAGCAAGACCACCGTGGCCAGCCTGATC | |
| | AACACCCTGAACGACCTGAACGAGACCCTGGTGACCATGCCCCTGGGC | |
| | TACGTGACCCACGGCCTGAACCTGGAGGAGGCCGCCAGGTACATGAG | |
| | GAGCCTGAAGGTGCCCGCCACCGTGAGCGTGAGCAGCCCCGACGCCG | |
| | TGACCGCCTACAACGGCTACCTGACCAGCAGCAGCAAGACCCCCGAG | |
| | GAGCACTTCATCGAGACCATCAGCCTGGCCGGCAGCTACAAGGACTGG | |
| | AGCTACAGCGGCCAGAGCACCCAGCTGGGCATCGAGTTCCTGAAGAG | |
| | GGGCGACAAGAGCGTGTACTACACCAGCAACCCCACCACCTTCCACCT | |
| | GGACGGCGAGGTGATCACCTTCGACAACCTGAAGACCCTGCTGAGCCT | |
| | GAGGGAGGTGAGGACCATCAAGGTGTTCACCACCGTGGACAACATCAA | |
| | CCTGCACTGATGACTCGAGCTGGTACTGCATGCACGCAATGCTAGCTG | |
| | CCCCTTTCCCGTCCTGGGTACCCCGAGTCTCCCCCGACCTCGGGTCCC | |
| | AGGTATGCTCCCACCTCCACCTGCCCCACTCACCACCTCTGCTAGTTCC | |
| | AGACACCTCCCAAGCACGCAGCAATGCAGCTCAAAACGCTTAGCCTAG | |
| | CCACACCCCCACGGGAAACAGCAGTGATTAACCTTTAGCAATAAACGAA | |
| | AGTTTAACTAAGCTATACTAACCCCAGGGTTGGTCAATTTCGTGCCAGC | |
| | CACACCCTGGAGCTAGCAAAAAAAA | |
| 52 | CTCGACATTGATTATTGACTAGTTATTAATAGTAATCAATTACGGGGTCA | 337 |
| Pan-CoV | TTAGTTCATAGCCCATATATGGAGTTCCGCGTTACATAACTTACGGTAAA | |
| rAdv5-CAG- | TGGCCCGCCTGGCTGACCGCCCAACGACCCCCGCCCATTGACGTCAAT | |
| NSP5 | AATGACGTATGTTCCCATAGTAACGCCAATAGGGACTTTCCATTGACGT | |
| | CAATGGGTGGAGTATTTACGGTAAACTGCCCACTTGGCAGTACATCAAG | |
| | TGTATCATATGCCAAGTACGCCCCCTATTGACGTCAATGACGGTAAATG | |
| | GCCCGCCTGGCATTATGCCCAGTACATGACCTTATGGGACTTTCCTACT | |
| | TGGCAGTACATCTACGTATTAGTCATCGCTATTACCATGGTCGAGGTGA | |
| | GCCCCACGTTCTGCTTCACTCTCCCCATCTCCCCCCCCTCCCCACCCC | |
| | CAATTTTGTATTTATTTATTTTTTAATTATTTTGTGCAGCGATGGGGGCG | |
| | GGGGGGGGGGGGGGGCGCGCGCCAGGCGGGGCGGGGCGGGGCGA | |
| | GGGGCGGGGCGGGGCGAGGCGGAGAGGTGCGGCGGCAGCCAATCA | |
| | GAGCGGCGCGCTCCGAAAGTTTCCTTTTATGGCGAGGCGGCGGCGGC | |
| | GGCGGCCCTATAAAAAGCGAAGCGCGCGGCGGGCGGGAGTCGCTGC | |
| | GCGCTGCCTTCGCCCCGTGCCCCGCTCCGCCGCCGCCTCGCGCCGCC | |
| | CGCCCCGGCTCTGACTGACCGCGTTACTCCCACAGGTGAGCGGGCGG | |
| | GACGGCCCTTCTCCTCCGGGCTGTAATTAGCGCTTGGTTTAATGACGG | |
| | CTTGTTTCTTTTCTGTGGCTGCGTGAAAGCCTTGAGGGGCTCCGGGAG | |
| | GGCCCTTTGTGCGGGGGGAGCGGCTCGGGGGGTGCGTGCGTGTGTG | |
| | TGTGCGTGGGGAGCGCCGCGTGCGGCTCCGCGCTGCCCGGCGGCTG | |
| | TGAGCGCTGCGGGCGCGGCGCGGGGCTTTGTGCGCTCCGCAGTGTGC | |
| | GCGAGGGGAGCGCGGCCGGGGGCGGTGCCCCGCGGTGCGGGGGGG | |
| | GCTGCGAGGGGAACAAAGGCTGCGTGCGGGGTGTGTGCGTGGGGGG | |
| | GTGAGCAGGGGGTGTGGGCGCGTCGGTCGGGCTGCAACCCCCCCTG | |
| | CACCCCCCTCCCCGAGTTGCTGAGCACGGCCCGGCTTCGGGTGCGGG | |
| | GCTCCGTACGGGGCGTGGCGCGGGGCTCGCCGTGCCGGGCGGGGGG | |
| | TGGCGGCAGGTGGGGGTGCCGGGCGGGGCGGGGCCGCCTCGGGCC | |
| | GGGGAGGGCTCGGGGGAGGGGCGCGGCGGCCCCCGGAGCGCCGGC | |
| | GGCTGTCGAGGCGCGGCGAGCCGCAGCCATTGCCTTTTATGGTAATCG | |
| | TGCGAGAGGGCGCAGGGACTTCCTTTGTCCCAAATCTGTGCGGAGCCG | |
| | AAATCTGGGAGGCGCCGCCGCACCCCCTCTAGCGGGCGCGGGGCGAA | |
| | GCGGTGCGGCGCCGGCAGGAAGGAAATGGGCGGGGAGGGCCTTCGT | |
| | GCGTCGCCGCGCCGCCGTCCCCTTCTCCCTCTCCAGCCTCGGGGCTG | |
| | TCCGCGGGGGGACGGCTGCCTTCGGGGGGGACGGGGCAGGGCGGG | |
| | GTTCGGCTTCTGGCGTGTGACCGGCGGCTCTAGAGCCTCTGCTAACCA | |
| | TGTTCATGCCTTCTTCTTTTTCCTACAGCTCCTGGGCAACGTGCTGGTT | |
| | ATTGTGCTGTCTCATCATTTTGGCAAAGAATTGGAGAATAAACTAGTATT | |
| | CTTCTGGTCCCCACAGACTCAGAGAGAACCCGCCACCATGAGCGGCTT | |
| | CAGGAAGATGGCCTTCCCCAGCGGCAAGGTGGAGGGCTGCATGGTGC | |
| | AGGTGACCTGCGGCACCACCACCCTGAACGGCCTGTGGCTGGACGAC | |
| | GTGGTGTACTGCCCCAGGCACGTGATCTGCACCAGCGAGGACATGCTG | |
| | AACCCCAACTACGAGGACCTGCTGATCAGGAAGAGCAACCACAACTTC | |
| | CTGGTGCAGGCCGGCAACGTGCAGCTGAGGGTGATCGGCCACAGCAT | |
| | GCAGAACTGCGTGCTGAAGCTGAAGGTGGACACCGCCAACCCCAAGA | |
| | CCCCCAAGTACAAGTTCGTGAGGATCCAGCCCGGCCAGACCTTCAGCG | |
| | TGCTGGCCTGCTACAACGGCAGCCCCAGCGGCGTGTACCAGTGCGCC | |
| | ATGAGGCCCAACTTCACCATCAAGGGCAGCTTCCTGAACGGCAGCTGC | |
| | GGCAGCGTGGGCTTCAACATCGACTACGACTGCGTGAGCTTCTGCTAC | |
| | ATGCACCACATGGAGCTGCCCACCGGCGTGCACGCCGGCACCGACCT | |
| | GGAGGGCAACTTCTACGGCCCCTTCGTGGACAGGCAGACCGCCCAGG | |
| | CCGCCGGCACCGACACCACCATCACCGTGAACGTGCTGGCCTGGCTG | |
| | TACGCCGCCGTGATCAACGGCGACAGGTGGTTCCTGAACAGGTTCACC | |
| | ACCACCCTGAACGACTTCAACCTGGTGGCCATGAAGTACAACTACGAG | |
| | CCCCTGACCCAGGACCACGTGGACATCCTGGGCCCCCTGAGCGCCCA | |
| | GACCGGCATCGCCGTGCTGGACATGTGCGCCAGCCTGAAGGAGCTGC | |
| | TGCAGAACGGCATGAACGGCAGGACCATCCTGGGCAGCGCCCTGCTG | |
| | GAGGACGAGTTCACCCCCTTCGACGTGGTGAGGCAGTGCAGCGGCGT | |
| | GACCTTCCAGTGATGACTCGAGCTGGTACTGCATGCACGCAATGCTAG | |
| | CTGCCCCTTTCCCGTCCTGGGTACCCCGAGTCTCCCCCGACCTCGGGT | |
| | CCCAGGTATGCTCCCACCTCCACCTGCCCCACTCACCACCTCTGCTAG | |
| | TTCCAGACACCTCCCAAGCACGCAGCAATGCAGCTCAAAACGCTTAGC | |
| | CTAGCCACACCCCCACGGGAAACAGCAGTGATTAACCTTTAGCAATAAA | |
| | CGAAAGTTTAACTAAGCTATACTAACCCCAGGGTTGGTCAATTTCGTGC | |
| | CAGCCACACCCTGGAGCTAGCAAAAAAAA | |
| 53 | CTCGACATTGATTATTGACTAGTTATTAATAGTAATCAATTACGGGGTCA | 338 |
| Pan-CoV | TTAGTTCATAGCCCATATATGGAGTTCCGCGTTACATAACTTACGGTAAA | |
| rAdv5-CAG- | TGGCCCGCCTGGCTGACCGCCCAACGACCCCCGCCCATTGACGTCAAT | |
| NSP12 | AATGACGTATGTTCCCATAGTAACGCCAATAGGGACTTTCCATTGACGT | |
| | CAATGGGTGGAGTATTTACGGTAAACTGCCCACTTGGCAGTACATCAAG | |
| | TGTATCATATGCCAAGTACGCCCCCTATTGACGTCAATGACGGTAAATG | |
| | GCCCGCCTGGCATTATGCCCAGTACATGACCTTATGGGACTTTCCTACT | |
| | TGGCAGTACATCTACGTATTAGTCATCGCTATTACCATGGTCGAGGTGA | |
| | GCCCCACGTTCTGCTTCACTCTCCCCATCTCCCCCCCCTCCCCACCCC | |
| | CAATTTGTATTTTATTTATTTTTTAATTATTTTGTGCAGCGATGGGGGCG | |
| | GGGGGGGGGGGGGGGCGCGCGCCAGGCGGGGCGGGGCGGGGCGA | |
| | GGGGCGGGGCGGGGCGAGGCGGAGAGGTGCGGCGGCAGCCAATCA | |
| | GAGCGGCGCGCTCCGAAAGTTTCCTTTTATGGCGAGGCGGCGGCGGC | |
| | GGCGGCCCTATAAAAAGCGAAGCGCGCGGCGGGCGGGAGTCGCTGC | |
| | GCGCTGCCTTCGCCCCGTGCCCCGCTCCGCCGCCGCCTCGCGCCGCC | |
| | CGCCCCGGCTCTGACTGACCGCGTTACTCCCACAGGTGAGCGGGCGG | |
| | GACGGCCCTTCTCCTCCGGGCTGTAATTAGCGCTTGGTTTAATGACGG | |
| | CTTGTTTCTTTTCTGTGGCTGCGTGAAAGCCTTGAGGGGCTCCGGGAG | |
| | GGCCCTTTGTGCGGGGGGAGCGGCTCGGGGGGTGCGTGCGTGTGTG | |
| | TGTGCGTGGGGAGCGCCGCGTGCGGCTCCGCGCTGCCCGGCGGCTG | |
| | TGAGCGCTGCGGGCGCGGCGCGGGGCTTTGTGCGCTCCGCAGTGTGC | |
| | GCGAGGGGAGCGCGGCCGGGGGCGGTGCCCCGCGGTGCGGGGGGG | |
| | GCTGCGAGGGGAACAAAGGCTGCGTGCGGGGTGTGTGCGTGGGGGG | |
| | GTGAGCAGGGGGTGTGGGCGCGTCGGTCGGGCTGCAACCCCCCCTG | |
| | CACCCCCCTCCCCGAGTTGCTGAGCACGGCCCGGCTTCGGGTGCGGG | |
| | GCTCCGTACGGGGCGTGGCGCGGGGCTCGCCGTGCCGGGCGGGGGG | |
| | TGGCGGCAGGTGGGGGTGCCGGGCGGGGCGGGGCCGCCTCGGGCC | |
| | GGGGAGGGCTCGGGGGAGGGGCGCGGCGGCCCCCGGAGCGCCGGC | |
| | GGCTGTCGAGGCGCGGCGAGCCGCAGCCATTGCCTTTTATGGTAATCG | |
| | TGCGAGAGGGCGCAGGGACTTCCTTTGTCCCAAATCTGTGCGGAGCCG | |
| | AAATCTGGGAGGCGCCGCCGCACCCCCTCTAGCGGGCGCGGGGCGAA | |
| | GCGGTGCGGCGCCGGCAGGAAGGAAATGGGCGGGGAGGGCCTTCGT | |
| | GCGTCGCCGCGCCGCCGTCCCCTTCTCCCTCTCCAGCCTCGGGGCTG | |
| | TCCGCGGGGGGACGGCTGCCTTCGGGGGGGACGGGGCAGGGCGGG | |
| | GTTCGGCTTCTGGCGTGTGACCGGCGGCTCTAGAGCCTCTGCTAACCA | |
| | TGTTCATGCCTTCTTCTTTTTCCTACAGCTCCTGGGCAACGTGCTGGTT | |
| | ATTGTGCTGTCTCATCATTTTGGCAAAGAATTGGAGAATAAACTAGTATT | |
| | CTTCTGGTCCCCACAGACTCAGAGAGAACCCGCCACCATGAGCGCCGA | |
| | CGCCCAGAGCTTCCTGAACAGGGTGTGCGGCGTGAGCGCCGCCAGGC | |
| | TGACCCCCTGCGGCACCGGCACCAGCACCGACGTGGTGTACAGGGCC | |
| | TTCGACATCTACAACGACAAGGTGGCCGGCTTCGCCAAGTTCCTGAAG | |
| | ACCAACTGCTGCAGGTTCCAGGAGAAGGACGAGGACGACAACCTGATC | |
| | GACAGCTACTTCGTGGTGAAGAGGCACACCTTCAGCAACTACCAGCAC | |
| | GAGGAGACCATCTACAACCTGCTGAAGGACTGCCCCGCCGTGGCCAA | |
| | GCACGACTTCTTCAAGTTCAGGATCGACGGCGACATGGTGCCCCACAT | |
| | CAGCAGGCAGAGGCTGACCAAGTACACCATGGCCGACCTGGTGTACG | |
| | CCCTGAGGCACTTCGACGAGGGCAACTGCGACACCCTGAAGGAGATC | |
| | CTGGTGACCTACAACTGCTGCGACGACGACTACTTCAACAAGAAGGAC | |
| | TGGTACGACTTCGTGGAGAACCCCGACATCCTGAGGGTGTACGCCAAC | |
| | CTGGGCGAGAGGGTGAGGCAGGCCCTGCTGAAGACCGTGCAGTTCTG | |
| | CGACGCCATGAGGAACGCCGGCATCGTGGGCGTGCTGACCCTGGACA | |
| | ACCAGGACCTGAACGGCAACTGGTACGACTTCGGCGACTTCATCCAGA | |
| | CCACCCCCGGCAGCGGCGTGCCCGTGGTGGACAGCTACTACAGCCTG | |
| | CTGATGCCCATCCTGACCCTGACCAGGGCCCTGACCGCCGAGAGCCA | |
| | CGTGGACACCGACCTGACCAAGCCCTACATCAAGTGGGACCTGCTGAA | |
| | GTACGACTTCACCGAGGAGAGGCTGAAGCTGTTCGACAGGTACTTCAA | |
| | GTACTGGGACCAGACCTACCACCCCAACTGCGTGAACTGCCTGGACGA | |
| | CAGGTGCATCCTGCACTGCGCCAACTTCAACGTGCTGTTCAGCACCGT | |
| | GTTCCCCCCCACCAGCTTCGGCCCCCTGGTGAGGAAGATCTTCGTGGA | |
| | CGGCGTGCCCTTCGTGGTGAGCACCGGCTACCACTTCAGGGAGCTGG | |
| | GCGTGGTGCACAACCAGGACGTGAACCTGCACAGCAGCAGGCTGAGC | |
| | TTCAAGGAGCTGCTGGTGTACGCCGCCGACCCCGCCATGCACGCCGC | |
| | CAGCGGCAACCTGCTGCTGGACAAGAGGACCACCTGCTTCAGCGTGG | |
| | CCGCCCTGACCAACAACGTGGCCTTCCAGACCGTGAAGCCCGGCAACT | |
| | TCAACAAGGACTTCTACGACTTCGCCGTGAGCAAGGGCTTCTTCAAGG | |
| | AGGGCAGCAGCGTGGAGCTGAAGCACTTCTTCTTCGCCCAGGACGGC | |
| | AACGCCGCCATCAGCGACTACGACTACTACAGGTACAACCTGCCCACC | |
| | ATGTGCGACATCAGGCAGCTGCTGTTCGTGGTGGAGGTGGTGGACAAG | |
| | TACTTCGACTGCTACGACGGCGGCTGCATCAACGCCAACCAGGTGATC | |
| | GTGAACAACCTGGACAAGAGCGCCGGCTTCCCCTTCAACAAGTGGGGC | |
| | AAGGCCAGGCTGTACTACGACAGCATGAGCTACGAGGACCAGGACGC | |
| | CCTGTTCGCCTACACCAAGAGGAACGTGATCCCCACCATCACCCAGAT | |
| | GAACCTGAAGTACGCCATCAGCGCCAAGAACAGGGCCAGGACCGTGG | |
| | CCGGCGTGAGCATCTGCAGCACCATGACCAACAGGCAGTTCCACCAGA | |
| | AGCTGCTGAAGAGCATCGCCGCCACCAGGGGCGCCACCGTGGTGATC | |
| | GGCACCAGCAAGTTCTACGGCGGCTGGCACAACATGCTGAAGACCGTG | |
| | TACAGCGACGTGGAGAACCCCCACCTGATGGGCTGGGACTACCCCAA | |
| | GTGCGACAGGGCCATGCCCAACATGCTGAGGATCATGGCCAGCCTGG | |
| | TGCTGGCCAGGAAGCACACCACCTGCTGCAGCCTGAGCCACAGGTTCT | |
| | ACAGGCTGGCCAACGAGTGCGCCCAGGTGCTGAGCGAGATGGTGATG | |
| | TGCGGCGGCAGCCTGTACGTGAAGCCCGGCGGCACCAGCAGCGGCG | |
| | ACGCCACCACCGCCTACGCCAACAGCGTGTTCAACATCTGCCAGGCCG | |
| | TGACCGCCAACGTGAACGCCCTGCTGAGCACCGACGGCAACAAGATC | |
| | GCCGACAAGTACGTGAGGAACCTGCAGCACAGGCTGTACGAGTGCCT | |
| | GTACAGGAACAGGGACGTGGACACCGACTTCGTGAACGAGTTCTACGC | |
| | CTACCTGAGGAAGCACTTCAGCATGATGATCCTGAGCGACGACGCCGT | |
| | GGTGTGCTTCAACAGCACCTACGCCAGCCAGGGCCTGGTGGCCAGCA | |
| | TCAAGAACTTCAAGAGCGTGCTGTACTACCAGAACAACGTGTTCATGAG | |
| | CGAGGCCAAGTGCTGGACCGAGACCGACCTGACCAAGGGCCCCCACG | |
| | AGTTCTGCAGCCAGCACACCATGCTGGTGAAGCAGGGCGACGACTACG | |
| | TGTACCTGCCCTACCCCGACCCCAGCAGGATCCTGGGCGCCGGCTGC | |
| | TTCGTGGACGACATCGTGAAGACCGACGGCACCCTGATGATCGAGAGG | |
| | TTCGTGAGCCTGGCCATCGACGCCTACCCCCTGACCAAGCACCCCAAC | |
| | CAGGAGTACGCCGACGTGTTCCACCTGTACCTGCAGTACATCAGGAAG | |
| | CTGCACGACGAGCTGACCGGCCACATGCTGGACATGTACAGCGTGATG | |
| | CTGACCAACGACAACACCAGCAGGTACTGGGAGCCCGAGTTCTACGAG | |
| | GCCATGTACACCCCCCACACCGTGCTGCAGTGATGACTCGAGCTGGTA | |
| | CTGCATGCACGCAATGCTAGCTGCCCCTTTCCCGTCCTGGGTACCCCG | |
| | AGTCTCCCCCGACCTCGGGTCCCAGGTATGCTCCCACCTCCACCTGCC | |
| | CCACTCACCACCTCTGCTAGTTCCAGACACCTCCCAAGCACGCAGCAA | |
| | TGCAGCTCAAAACGCTTAGCCTAGCCACACCCCCACGGGAAACAGCAG | |
| | TGATTAACCTTTAGCAATAAACGAAAGTTTAACTAAGCTATACTAACCCC | |
| | AGGGTTGGTCAATTTCGTGCCAGCCACACCCTGGAGCTAGCAAAAAAA | |
| | A | |
| 54 | ATGTTCGTGTTCCTGGTGCTGCTGCCCCTGGTGAGCAGCCAGTGCGTG | 339 |
| Pan-CoV (B1) | AACCTGACCACCAGGACCCAGCTGCCCCCCGCCTACACCAACAGCTTC | |
| | ACCAGGGGCGTGTACTACCCCGACAAGGTGTTCAGGAGCAGCGTGCT | |
| | GCACAGCACCCAGGACCTGTTCCTGCCCTTCTTCAGCAACGTGACCTG | |
| | GTTCCACGCCATCCACGTGAGCGGCACCAACGGCACCAAGAGGTTCGA | |
| | CAACCCCGTGCTGCCCTTCAACGACGGCGTGTACTTCGCCAGCACCGA | |
| | GAAGAGCAACATCATCAGGGGCTGGATCTTCGGCACCACCCTGGACAG | |
| | CAAGACCCAGAGCCTGCTGATCGTGAACAACGCCACCAACGTGGTGAT | |
| | CAAGGTGTGCGAGTTCCAGTTCTGCAACGACCCCTTCCTGGGCGTGTA | |
| | CTACCACAAGAACAACAAGAGCTGGATGGAGAGCGAGTTCAGGGTGTA | |
| | CAGCAGCGCCAACAACTGCACCTTCGAGTACGTGAGCCAGCCCTTCCT | |
| | GATGGACCTGGAGGGCAAGCAGGGCAACTTCAAGAACCTGAGGGAGT | |
| | TCGTGTTCAAGAACATCGACGGCTACTTCAAGATCTACAGCAAGCACAC | |
| | CCCCATCAACCTGGTGAGGGACCTGCCCCAGGGCTTCAGCGCCCTGG | |
| | AGCCCCTGGTGGACCTGCCCATCGGCATCAACATCACCAGGTTCCAGA | |
| | CCCTGCTGGCCCTGCACAGGAGCTACCTGACCCCCGGCGACAGCAGC | |
| | AGCGGCTGGACCGCCGGCGCCGCCGCCTACTACGTGGGCTACCTGCA | |
| | GCCCAGGACCTTCCTGCTGAAGTACAACGAGAACGGCACCATCACCGA | |
| | CGCCGTGGACTGCGCCCTGGACCCCCTGAGCGAGACCAAGTGCACCC | |
| | TGAAGAGCTTCACCGTGGAGAAGGGCATCTACCAGACCAGCAACTTCA | |
| | GGGTGCAGCCCACCGAGAGCATCGTGAGGTTCCCCAACATCACCAACC | |
| | TGTGCCCCTTCGGCGAGGTGTTCAACGCCACCAGGTTCGCCAGCGTGT | |
| | ACGCCTGGAACAGGAAGAGGATCAGCAACTGCGTGGCCGACTACAGC | |
| | GTGCTGTACAACAGCGCCAGCTTCAGCACCTTCAAGTGCTACGGCGTG | |
| | AGCCCCACCAAGCTGAACGACCTGTGCTTCACCAACGTGTACGCCGAC | |
| | AGCTTCGTGATCAGGGGCGACGAGGTGAGGCAGATCGCCCCCGGCCA | |
| | GACCGGCAAGATCGCCGACTACAACTACAAGCTGCCCGACGACTTCAC | |
| | CGGCTGCGTGATCGCCTGGAACAGCAACAACCTGGACAGCAAGGTGG | |
| | GCGGCAACTACAACTACCTGTACAGGCTGTTCAGGAAGAGCAACCTGA | |
| | AGCCCTTCGAGAGGGACATCAGCACCGAGATCTACCAGGCCGGCAGC | |
| | ACCCCCTGCAACGGCGTGGAGGGCTTCAACTGCTACTTCCCCCTGCAG | |
| | AGCTACGGCTTCCAGCCCACCAACGGCGTGGGCTACCAGCCCTACAG | |
| | GGTGGTGGTGCTGAGCTTCGAGCTGCTGCACGCCCCCGCCACCGTGT | |
| | GCGGCCCCAAGAAGAGCACCAACCTGGTGAAGAACAAGTGCGTGAACT | |
| | TCAACTTCAACGGCCTGACCGGCACCGGCGTGCTGACCGAGAGCAACA | |
| | AGAAGTTCCTGCCCTTCCAGCAGTTCGGCAGGGACATCGCCGACACCA | |
| | CCGACGCCGTGAGGGACCCCCAGACCCTGGAGATCCTGGACATCACC | |
| | CCCTGCAGCTTCGGCGGCGTGAGCGTGATCACCCCCGGCACCAACAC | |
| | CAGCAACCAGGTGGCCGTGCTGTACCAGGACGTGAACTGCACCGAGG | |
| | TGCCCGTGGCCATCCACGCCGACCAGCTGACCCCCACCTGGAGGGTG | |
| | TACAGCACCGGCAGCAACGTGTTCCAGACCAGGGCCGGCTGCCTGAT | |
| | CGGCGCCGAGCACGTGAACAACAGCTACGAGTGCGACATCCCCATCG | |
| | GCGCCGGCATCTGCGCCAGCTACCAGACCCAGACCAACAGCCCCGGC | |
| | AGCGCCAGCAGCGTGGCCAGCCAGAGCATCATCGCCTACACCATGAG | |
| | CCTGGGCGCCGAGAACAGCGTGGCCTACAGCAACAACAGCATCGCCA | |
| | TCCCCACCAACTTCACCATCAGCGTGACCACCGAGATCCTGCCCGTGA | |
| | GCATGACCAAGACCAGCGTGGACTGCACCATGTACATCTGCGGCGACA | |
| | GCACCGAGTGCAGCAACCTGCTGCTGCAGTACGGCAGCTTCTGCACCC | |
| | AGCTGAACAGGGCCCTGACCGGCATCGCCGTGGAGCAGGACAAGAAC | |
| | ACCCAGGAGGTGTTCGCCCAGGTGAAGCAGATCTACAAGACCCCCCCC | |
| | ATCAAGGACTTCGGCGGCTTCAACTTCAGCCAGATCCTGCCCGACCCC | |
| | AGCAAGCCCAGCAAGAGGAGCCCCATCGAGGACCTGCTGTTCAACAAG | |
| | GTGACCCTGGCCGACGCCGGCTTCATCAAGCAGTACGGCGACTGCCT | |
| | GGGCGACATCGCCGCCAGGGACCTGATCTGCGCCCAGAAGTTCAACG | |
| | GCCTGACCGTGCTGCCCCCCCTGCTGACCGACGAGATGATCGCCCAG | |
| | TACACCAGCGCCCTGCTGGCCGGCACCATCACCAGCGGCTGGACCTT | |
| | CGGCGCCGGCCCCGCCCTGCAGATCCCCTTCCCCATGCAGATGGCCT | |
| | ACAGGTTCAACGGCATCGGCGTGACCCAGAACGTGCTGTACGAGAACC | |
| | AGAAGCTGATCGCCAACCAGTTCAACAGCGCCATCGGCAAGATCCAGG | |
| | ACAGCCTGAGCAGCACCCCCAGCGCCCTGGGCAAGCTGCAGGACGTG | |
| | GTGAACCAGAACGCCCAGGCCCTGAACACCCTGGTGAAGCAGCTGAG | |
| | CAGCAACTTCGGCGCCATCAGCAGCGTGCTGAACGACATCCTGAGCAG | |
| | GCTGGACCCCCCCGAGGCCGAGGTGCAGATCGACAGGCTGATCACCG | |
| | GCAGGCTGCAGAGCCTGCAGACCTACGTGACCCAGCAGCTGATCAGG | |
| | GCCGCCGAGATCAGGGCCAGCGCCAACCTGGCCGCCACCAAGATGAG | |
| | CGAGTGCGTGCTGGGCCAGAGCAAGAGGGTGGACTTCTGCGGCAAGG | |
| | GCTACCACCTGATGAGCTTCCCCCAGAGCGCCCCCCACGGCGTGGTG | |
| | TTCCTGCACGTGACCTACGTGCCCGCCCAGGAGAAGAACTTCACCACC | |
| | GCCCCCGCCATCTGCCACGACGGCAAGGCCCACTTCCCCAGGGAGGG | |
| | CGTGTTCGTGAGCAACGGCACCCACTGGTTCGTGACCCAGAGGAACTT | |
| | CTACGAGCCCCAGATCATCACCACCGACAACACCTTCGTGAGCGGCAA | |
| | CTGCGACGTGGTGATCGGCATCGTGAACAACACCGTGTACGACCCCCT | |
| | GCAGCCCGAGCTGGACAGCTTCAAGGAGGAGCTGGACAAGTACTTCAA | |
| | GAACCACACCAGCCCCGACGTGGACCTGGGCGACATCAGCGGCATCA | |
| | ACGCCAGCGTGGTGAACATCCAGAAGGAGATCGACAGGCTGAACGAG | |
| | GTGGCCAAGAACCTGAACGAGAGCCTGATCGACCTGCAGGAGCTGGG | |
| | CAAGTACGAGCAGTACATCAAGTGGCCCTGGTACATCTGGCTGGGCTT | |
| | CATCGCCGGCCTGATCGCCATCGTGATGGTGACCATCATGCTGTGCTG | |
| | CATGACCAGCTGCTGCAGCTGCCTGAAGGGCTGCTGCAGCTGCGGCA | |
| | GCTGCTGCAAGTTCGACGAGGACGACAGCGAGCCCGTGCTGAAGGGC | |
| | GTGAAGCTGCACTACACCGGAAGCGGAGCCACGAACTTCTCTCTGTTA | |
| | AAGCAAGCAGGAGATGTTGAAGAAAACCCCGGGCCTATGAGCGACAAC | |
| | GGCCCCCAGAACCAGAGGAACGCCCCCAGGATCACCTTCGGCGGCCC | |
| | CAGCGACAGCACCGGCAGCAACCAGAACGGCGAGAGGAGCGGCGCC | |
| | AGGAGCAAGCAGAGGAGGCCCCAGGGCCTGCCCAACAACACCGCCAG | |
| | CTGGTTCACCGCCCTGACCCAGCACGGCAAGGAGGACCTGAAGTTCCC | |
| | CAGGGGCCAGGGCGTGCCCATCAACACCAACAGCAGCCCCGACGACC | |
| | AGATCGGCTACTACAGGAGGGCCACCAGGAGGATCAGGGGCGGCGAC | |
| | GGCAAGATGAAGGACCTGAGCCCCAGGTGGTACTTCTACTACCTGGGC | |
| | ACCGGCCCCGAGGCCGGCCTGCCCTACGGCGCCAACAAGGACGGCAT | |
| | CATCTGGGTGGCCACCGAGGGCGCCCTGAACACCCCCAAGGACCACA | |
| | TCGGCACCAGGAACCCCGCCAACAACGCCGCCATCGTGCTGCAGCTG | |
| | CCCCAGGGCACCACCCTGCCCAAGGGCTTCTACGCCGAGGGCAGCAG | |
| | GGGCGGCAGCCAGGCCAGCAGCAGGAGCAGCAGCAGGAGCAGGAAC | |
| | AGCAGCAGGAACAGCACCCCCGGCAGCAGCAGGGGCACCAGCCCCG | |
| | CCAGGATGGCCGGCAACGGCGGCGACGCCGCCCTGGCCCTGCTGCT | |
| | GCTGGACAGGCTGAACCAGCTGGAGAGCAAGATGAGCGGCAAGGGCC | |
| | AGCAGCAGCAGGGCCAGACCGTGACCAAGAAGAGCGCCGCCGAGGCC | |
| | AGCAAGAAGCCCAGGCAGAAGAGGACCGCCACCAAGGCCTACAACGT | |
| | GACCCAGGCCTTCGGCAGGAGGGGCCCCGAGCAGACCCAGGGCAACT | |
| | TCGGCGACCAGGAGCTGATCAGGCAGGGCACCGACTACAAGCACTGG | |
| | CCCCAGATCGCCCAGTTCGCCCCCAGCGCCAGCGCCTTCTTCGGCATG | |
| | AGCAGGATCGGCATGGAGGTGACCCCCAGCGGCACCTGGCTGACCTA | |
| | CACCGGCGCCATCAAGCTGGACGACAAGGACCCCAACTTCAAGGACCA | |
| | GGTGATCCTGCTGAACAAGCACATCGACGCCTACAAGACCTTCCCCCC | |
| | CACCGAGCCCAAGAAGGACAAGAAGAAGAAGGCCGACGAGACCCAGG | |
| | CCCTGCCCCAGAGGCAGAAGAAGCAGCAGACCGTGACCCTGCTGCCC | |
| | GCCGCCGACCTGGACGACTTCAGCAAGCAGCTGCAGCAGAGCATGAG | |
| | CAGCGCCGACAGCACCCAGGCCTGATGA | |
| 55 | ATGTTCGTGTTCCTGGTGCTGCTGCCCCTGGTGAGCAGCCAGTGCGTG | 348 |
| Pan-CoV (B2) | AACCTGACCACCAGGACCCAGCTGCCCCCCGCCTACACCAACAGCTTC | |
| | ACCAGGGGCGTGTACTACCCCGACAAGGTGTTCAGGAGCAGCGTGCT | |
| | GCACAGCACCCAGGACCTGTTCCTGCCCTTCTTCAGCAACGTGACCTG | |
| | GTTCCACGCCATCCACGTGAGCGGCACCAACGGCACCAAGAGGTTCGA | |
| | CAACCCCGTGCTGCCCTTCAACGACGGCGTGTACTTCGCCAGCACCGA | |
| | GAAGAGCAACATCATCAGGGGCTGGATCTTCGGCACCACCCTGGACAG | |
| | CAAGACCCAGAGCCTGCTGATCGTGAACAACGCCACCAACGTGGTGAT | |
| | CAAGGTGTGCGAGTTCCAGTTCTGCAACGACCCCTTCCTGGGCGTGTA | |
| | CTACCACAAGAACAACAAGAGCTGGATGGAGAGCGAGTTCAGGGTGTA | |
| | CAGCAGCGCCAACAACTGCACCTTCGAGTACGTGAGCCAGCCCTTCCT | |
| | GATGGACCTGGAGGGCAAGCAGGGCAACTTCAAGAACCTGAGGGAGT | |
| | TCGTGTTCAAGAACATCGACGGCTACTTCAAGATCTACAGCAAGCACAC | |
| | CCCCATCAACCTGGTGAGGGACCTGCCCCAGGGCTTCAGCGCCCTGG | |
| | AGCCCCTGGTGGACCTGCCCATCGGCATCAACATCACCAGGTTCCAGA | |
| | CCCTGCTGGCCCTGCACAGGAGCTACCTGACCCCCGGCGACAGCAGC | |
| | AGCGGCTGGACCGCCGGCGCCGCCGCCTACTACGTGGGCTACCTGCA | |
| | GCCCAGGACCTTCCTGCTGAAGTACAACGAGAACGGCACCATCACCGA | |
| | CGCCGTGGACTGCGCCCTGGACCCCCTGAGCGAGACCAAGTGCACCC | |
| | TGAAGAGCTTCACCGTGGAGAAGGGCATCTACCAGACCAGCAACTTCA | |
| | GGGTGCAGCCCACCGAGAGCATCGTGAGGTTCCCCAACATCACCAACC | |
| | TGTGCCCCTTCGGCGAGGTGTTCAACGCCACCAGGTTCGCCAGCGTGT | |
| | ACGCCTGGAACAGGAAGAGGATCAGCAACTGCGTGGCCGACTACAGC | |
| | GTGCTGTACAACAGCGCCAGCTTCAGCACCTTCAAGTGCTACGGCGTG | |
| | AGCCCCACCAAGCTGAACGACCTGTGCTTCACCAACGTGTACGCCGAC | |
| | AGCTTCGTGATCAGGGGCGACGAGGTGAGGCAGATCGCCCCCGGCCA | |
| | GACCGGCAAGATCGCCGACTACAACTACAAGCTGCCCGACGACTTCAC | |
| | CGGCTGCGTGATCGCCTGGAACAGCAACAACCTGGACAGCAAGGTGG | |
| | GCGGCAACTACAACTACCTGTACAGGCTGTTCAGGAAGAGCAACCTGA | |
| | AGCCCTTCGAGAGGGACATCAGCACCGAGATCTACCAGGCCGGCAGC | |
| | ACCCCCTGCAACGGCGTGGAGGGCTTCAACTGCTACTTCCCCCTGCAG | |
| | AGCTACGGCTTCCAGCCCACCAACGGCGTGGGCTACCAGCCCTACAG | |
| | GGTGGTGGTGCTGAGCTTCGAGCTGCTGCACGCCCCCGCCACCGTGT | |
| | GCGGCCCCAAGAAGAGCACCAACCTGGTGAAGAACAAGTGCGTGAACT | |
| | TCAACTTCAACGGCCTGACCGGCACCGGCGTGCTGACCGAGAGCAACA | |
| | AGAAGTTCCTGCCCTTCCAGCAGTTCGGCAGGGACATCGCCGACACCA | |
| | CCGACGCCGTGAGGGACCCCCAGACCCTGGAGATCCTGGACATCACC | |
| | CCCTGCAGCTTCGGCGGCGTGAGCGTGATCACCCCCGGCACCAACAC | |
| | CAGCAACCAGGTGGCCGTGCTGTACCAGGACGTGAACTGCACCGAGG | |
| | TGCCCGTGGCCATCCACGCCGACCAGCTGACCCCCACCTGGAGGGTG | |
| | TACAGCACCGGCAGCAACGTGTTCCAGACCAGGGCCGGCTGCCTGAT | |
| | CGGCGCCGAGCACGTGAACAACAGCTACGAGTGCGACATCCCCATCG | |
| | GCGCCGGCATCTGCGCCAGCTACCAGACCCAGACCAACAGCCCCGGC | |
| | AGCGCCAGCAGCGTGGCCAGCCAGAGCATCATCGCCTACACCATGAG | |
| | CCTGGGCGCCGAGAACAGCGTGGCCTACAGCAACAACAGCATCGCCA | |
| | TCCCCACCAACTTCACCATCAGCGTGACCACCGAGATCCTGCCCGTGA | |
| | GCATGACCAAGACCAGCGTGGACTGCACCATGTACATCTGCGGCGACA | |
| | GCACCGAGTGCAGCAACCTGCTGCTGCAGTACGGCAGCTTCTGCACCC | |
| | AGCTGAACAGGGCCCTGACCGGCATCGCCGTGGAGCAGGACAAGAAC | |
| | ACCCAGGAGGTGTTCGCCCAGGTGAAGCAGATCTACAAGACCCCCCCC | |
| | ATCAAGGACTTCGGCGGCTTCAACTTCAGCCAGATCCTGCCCGACCCC | |
| | AGCAAGCCCAGCAAGAGGAGCCCCATCGAGGACCTGCTGTTCAACAAG | |
| | GTGACCCTGGCCGACGCCGGCTTCATCAAGCAGTACGGCGACTGCCT | |
| | GGGCGACATCGCCGCCAGGGACCTGATCTGCGCCCAGAAGTTCAACG | |
| | GCCTGACCGTGCTGCCCCCCCTGCTGACCGACGAGATGATCGCCCAG | |
| | TACACCAGCGCCCTGCTGGCCGGCACCATCACCAGCGGCTGGACCTT | |
| | CGGCGCCGGCCCCGCCCTGCAGATCCCCTTCCCCATGCAGATGGCCT | |
| | ACAGGTTCAACGGCATCGGCGTGACCCAGAACGTGCTGTACGAGAACC | |
| | AGAAGCTGATCGCCAACCAGTTCAACAGCGCCATCGGCAAGATCCAGG | |
| | ACAGCCTGAGCAGCACCCCCAGCGCCCTGGGCAAGCTGCAGGACGTG | |
| | GTGAACCAGAACGCCCAGGCCCTGAACACCCTGGTGAAGCAGCTGAG | |
| | CAGCAACTTCGGCGCCATCAGCAGCGTGCTGAACGACATCCTGAGCAG | |
| | GCTGGACCCCCCCGAGGCCGAGGTGCAGATCGACAGGCTGATCACCG | |
| | GCAGGCTGCAGAGCCTGCAGACCTACGTGACCCAGCAGCTGATCAGG | |
| | GCCGCCGAGATCAGGGCCAGCGCCAACCTGGCCGCCACCAAGATGAG | |
| | CGAGTGCGTGCTGGGCCAGAGCAAGAGGGTGGACTTCTGCGGCAAGG | |
| | GCTACCACCTGATGAGCTTCCCCCAGAGCGCCCCCCACGGCGTGGTG | |
| | TTCCTGCACGTGACCTACGTGCCCGCCCAGGAGAAGAACTTCACCACC | |
| | GCCCCCGCCATCTGCCACGACGGCAAGGCCCACTTCCCCAGGGAGGG | |
| | CGTGTTCGTGAGCAACGGCACCCACTGGTTCGTGACCCAGAGGAACTT | |
| | CTACGAGCCCCAGATCATCACCACCGACAACACCTTCGTGAGCGGCAA | |
| | CTGCGACGTGGTGATCGGCATCGTGAACAACACCGTGTACGACCCCCT | |
| | GCAGCCCGAGCTGGACAGCTTCAAGGAGGAGCTGGACAAGTACTTCAA | |
| | GAACCACACCAGCCCCGACGTGGACCTGGGCGACATCAGCGGCATCA | |
| | ACGCCAGCGTGGTGAACATCCAGAAGGAGATCGACAGGCTGAACGAG | |
| | GTGGCCAAGAACCTGAACGAGAGCCTGATCGACCTGCAGGAGCTGGG | |
| | CAAGTACGAGCAGTACATCAAGTGGCCCTGGTACATCTGGCTGGGCTT | |
| | CATCGCCGGCCTGATCGCCATCGTGATGGTGACCATCATGCTGTGCTG | |
| | CATGACCAGCTGCTGCAGCTGCCTGAAGGGCTGCTGCAGCTGCGGCA | |
| | GCTGCTGCAAGTTCGACGAGGACGACAGCGAGCCCGTGCTGAAGGGC | |
| | GTGAAGCTGCACTACACCTGATGA | |
| 56 | ATGTTCGTGTTCCTGGTGCTGCTGCCCCTGGTGAGCAGCCAGTGCGTG | 341 |
| Pan-CoV (B4) | AACCTGACCACCAGGACCCAGCTGCCCCCCGCCTACACCAACAGCTTC | |
| | ACCAGGGGCGTGTACTACCCCGACAAGGTGTTCAGGAGCAGCGTGCT | |
| | GCACAGCACCCAGGACCTGTTCCTGCCCTTCTTCAGCAACGTGACCTG | |
| | GTTCCACGCCATCCACGTGAGCGGCACCAACGGCACCAAGAGGTTCGA | |
| | CAACCCCGTGCTGCCCTTCAACGACGGCGTGTACTTCGCCAGCACCGA | |
| | GAAGAGCAACATCATCAGGGGCTGGATCTTCGGCACCACCCTGGACAG | |
| | CAAGACCCAGAGCCTGCTGATCGTGAACAACGCCACCAACGTGGTGAT | |
| | CAAGGTGTGCGAGTTCCAGTTCTGCAACGACCCCTTCCTGGGCGTGTA | |
| | CTACCACAAGAACAACAAGAGCTGGATGGAGAGCGAGTTCAGGGTGTA | |
| | CAGCAGCGCCAACAACTGCACCTTCGAGTACGTGAGCCAGCCCTTCCT | |
| | GATGGACCTGGAGGGCAAGCAGGGCAACTTCAAGAACCTGAGGGAGT | |
| | TCGTGTTCAAGAACATCGACGGCTACTTCAAGATCTACAGCAAGCACAC | |
| | CCCCATCAACCTGGTGAGGGACCTGCCCCAGGGCTTCAGCGCCCTGG | |
| | AGCCCCTGGTGGACCTGCCCATCGGCATCAACATCACCAGGTTCCAGA | |
| | CCCTGCTGGCCCTGCACAGGAGCTACCTGACCCCCGGCGACAGCAGC | |
| | AGCGGCTGGACCGCCGGCGCCGCCGCCTACTACGTGGGCTACCTGCA | |
| | GCCCAGGACCTTCCTGCTGAAGTACAACGAGAACGGCACCATCACCGA | |
| | CGCCGTGGACTGCGCCCTGGACCCCCTGAGCGAGACCAAGTGCACCC | |
| | TGAAGAGCTTCACCGTGGAGAAGGGCATCTACCAGACCAGCAACTTCA | |
| | GGGTGCAGCCCACCGAGAGCATCGTGAGGTTCCCCAACATCACCAACC | |
| | TGTGCCCCTTCGGCGAGGTGTTCAACGCCACCAGGTTCGCCAGCGTGT | |
| | ACGCCTGGAACAGGAAGAGGATCAGCAACTGCGTGGCCGACTACAGC | |
| | GTGCTGTACAACAGCGCCAGCTTCAGCACCTTCAAGTGCTACGGCGTG | |
| | AGCCCCACCAAGCTGAACGACCTGTGCTTCACCAACGTGTACGCCGAC | |
| | AGCTTCGTGATCAGGGGCGACGAGGTGAGGCAGATCGCCCCCGGCCA | |
| | GACCGGCAAGATCGCCGACTACAACTACAAGCTGCCCGACGACTTCAC | |
| | CGGCTGCGTGATCGCCTGGAACAGCAACAACCTGGACAGCAAGGTGG | |
| | GCGGCAACTACAACTACCTGTACAGGCTGTTCAGGAAGAGCAACCTGA | |
| | AGCCCTTCGAGAGGGACATCAGCACCGAGATCTACCAGGCCGGCAGC | |
| | ACCCCCTGCAACGGCGTGGAGGGCTTCAACTGCTACTTCCCCCTGCAG | |
| | AGCTACGGCTTCCAGCCCACCAACGGCGTGGGCTACCAGCCCTACAG | |
| | GGTGGTGGTGCTGAGCTTCGAGCTGCTGCACGCCCCCGCCACCGTGT | |
| | GCGGCCCCAAGAAGAGCACCAACCTGGTGAAGAACAAGTGCGTGAACT | |
| | TCAACTTCAACGGCCTGACCGGCACCGGCGTGCTGACCGAGAGCAACA | |
| | AGAAGTTCCTGCCCTTCCAGCAGTTCGGCAGGGACATCGCCGACACCA | |
| | CCGACGCCGTGAGGGACCCCCAGACCCTGGAGATCCTGGACATCACC | |
| | CCCTGCAGCTTCGGCGGCGTGAGCGTGATCACCCCCGGCACCAACAC | |
| | CAGCAACCAGGTGGCCGTGCTGTACCAGGACGTGAACTGCACCGAGG | |
| | TGCCCGTGGCCATCCACGCCGACCAGCTGACCCCCACCTGGAGGGTG | |
| | TACAGCACCGGCAGCAACGTGTTCCAGACCAGGGCCGGCTGCCTGAT | |
| | CGGCGCCGAGCACGTGAACAACAGCTACGAGTGCGACATCCCCATCG | |
| | GCGCCGGCATCTGCGCCAGCTACCAGACCCAGACCAACAGCCCCGGC | |
| | AGCGCCAGCAGCGTGGCCAGCCAGAGCATCATCGCCTACACCATGAG | |
| | CCTGGGCGCCGAGAACAGCGTGGCCTACAGCAACAACAGCATCGCCA | |
| | TCCCCACCAACTTCACCATCAGCGTGACCACCGAGATCCTGCCCGTGA | |
| | GCATGACCAAGACCAGCGTGGACTGCACCATGTACATCTGCGGCGACA | |
| | GCACCGAGTGCAGCAACCTGCTGCTGCAGTACGGCAGCTTCTGCACCC | |
| | AGCTGAACAGGGCCCTGACCGGCATCGCCGTGGAGCAGGACAAGAAC | |
| | ACCCAGGAGGTGTTCGCCCAGGTGAAGCAGATCTACAAGACCCCCCCC | |
| | ATCAAGGACTTCGGCGGCTTCAACTTCAGCCAGATCCTGCCCGACCCC | |
| | AGCAAGCCCAGCAAGAGGAGCCCCATCGAGGACCTGCTGTTCAACAAG | |
| | GTGACCCTGGCCGACGCCGGCTTCATCAAGCAGTACGGCGACTGCCT | |
| | GGGCGACATCGCCGCCAGGGACCTGATCTGCGCCCAGAAGTTCAACG | |
| | GCCTGACCGTGCTGCCCCCCCTGCTGACCGACGAGATGATCGCCCAG | |
| | TACACCAGCGCCCTGCTGGCCGGCACCATCACCAGCGGCTGGACCTT | |
| | CGGCGCCGGCCCCGCCCTGCAGATCCCCTTCCCCATGCAGATGGCCT | |
| | ACAGGTTCAACGGCATCGGCGTGACCCAGAACGTGCTGTACGAGAACC | |
| | AGAAGCTGATCGCCAACCAGTTCAACAGCGCCATCGGCAAGATCCAGG | |
| | ACAGCCTGAGCAGCACCCCCAGCGCCCTGGGCAAGCTGCAGGACGTG | |
| | GTGAACCAGAACGCCCAGGCCCTGAACACCCTGGTGAAGCAGCTGAG | |
| | CAGCAACTTCGGCGCCATCAGCAGCGTGCTGAACGACATCCTGAGCAG | |
| | GCTGGACCCCCCCGAGGCCGAGGTGCAGATCGACAGGCTGATCACCG | |
| | GCAGGCTGCAGAGCCTGCAGACCTACGTGACCCAGCAGCTGATCAGG | |
| | GCCGCCGAGATCAGGGCCAGCGCCAACCTGGCCGCCACCAAGATGAG | |
| | CGAGTGCGTGCTGGGCCAGAGCAAGAGGGTGGACTTCTGCGGCAAGG | |
| | GCTACCACCTGATGAGCTTCCCCCAGAGCGCCCCCCACGGCGTGGTG | |
| | TTCCTGCACGTGACCTACGTGCCCGCCCAGGAGAAGAACTTCACCACC | |
| | GCCCCCGCCATCTGCCACGACGGCAAGGCCCACTTCCCCAGGGAGGG | |
| | CGTGTTCGTGAGCAACGGCACCCACTGGTTCGTGACCCAGAGGAACTT | |
| | CTACGAGCCCCAGATCATCACCACCGACAACACCTTCGTGAGCGGCAA | |
| | CTGCGACGTGGTGATCGGCATCGTGAACAACACCGTGTACGACCCCCT | |
| | GCAGCCCGAGCTGGACAGCTTCAAGGAGGAGCTGGACAAGTACTTCAA | |
| | GAACCACACCAGCCCCGACGTGGACCTGGGCGACATCAGCGGCATCA | |
| | ACGCCAGCGTGGTGAACATCCAGAAGGAGATCGACAGGCTGAACGAG | |
| | GTGGCCAAGAACCTGAACGAGAGCCTGATCGACCTGCAGGAGCTGGG | |
| | CAAGTACGAGCAGTACATCAAGTGGCCCTGGTACATCTGGCTGGGCTT | |
| | CATCGCCGGCCTGATCGCCATCGTGATGGTGACCATCATGCTGTGCTG | |
| | CATGACCAGCTGCTGCAGCTGCCTGAAGGGCTGCTGCAGCTGCGGCA | |
| | GCTGCTGCAAGTTCGACGAGGACGACAGCGAGCCCGTGCTGAAGGGC | |
| | GTGAAGCTGCACTACACCGGAAGCGGAGCCACGAACTTCTCTCTGTTA | |
| | AAGCAAGCAGGAGATGTTGAAGAAAACCCCGGGCCTGACCAGGAACCC | |
| | CGCCTGGAGGAAGGCCGTGTTCATCAGCCCCTACAACAGCCAGAACGC | |
| | CGTGGCCAGCAAGATCCTGGGCCTGCCCACCCAGACCGTGGACAGCA | |
| | GCCAGGGCAGCGAGTACGACTACGTGATCTTCACCCAGACCACCGAGA | |
| | CCGCCCACAGCTGCAACGTGAACAGGTTCAACGTGGCCATCACCAGGG | |
| | CCAAGGTGGGCATCCTGTGCATCATGAGCGACAGGGACCTGTACGACA | |
| | AGCTGCAGTTCACCAGCCTGGAGATCCCCAGGAGGAACGTGGCCACC | |
| | CTGCAGGCCGAGAACGTGACCGGCCTGTTCAAGGACTGCAGCAAGGT | |
| | GATCACCGGCCTGCACCCCACCCAGGCCCCCACCCACCTGAGCGTGG | |
| | ACACCAAGTTCAAGACCGAGGGCCTGTGCGTGGACATCCCCGGCATCC | |
| | CCAAGGACATGACCTACAGGAGGCTGATCAGCATGATGGGCTTCAAGA | |
| | TGAACTACCAGGTGAACGGCTACCCCAACATGTTCATCACCAGGGAGG | |
| | AGGCCATCAGGCACGTGAGGGCCTGGATCGGCTTCGACGTGGAGGGC | |
| | TGCCACGCCACCAGGGAGGCCGTGGGCACCAACCTGCCCCTGCAGCT | |
| | GGGCTTCAGCACCGGCGTGAACCTGGTGGCCGTGCCCACCGGCTACG | |
| | TGGACACCCCCAACAACACCGACTTCAGCAGGGTGAGCGCCAAGCCC | |
| | CCCCCCGGCGACCAGTTCAAGCACCTGATCCCCCTGATGTACAAGGGC | |
| | CTGCCCTGGAACGTGGTGAGGATCAAGATCGTGCAGATGCTGAGCGAC | |
| | ACCCTGAAGAACCTGAGCGACAGGGTGGTGTTCGTGCTGTGGGCCCA | |
| | CGGCTTCGAGCTGACCAGCATGAAGTACTTCGTGAAGATCGGCCCCGA | |
| | GAGGACCTGCTGCCTGTGCGACAGGAGGGCCACCTGCTTCAGCACCG | |
| | CCAGCGACACCTACGCCTGCTGGCACCACAGCATCGGCTTCGACTACG | |
| | TGTACAACCCCTTCATGATCGACGTGCAGCAGTGGGGCTTCACCGGCA | |
| | ACCTGCAGAGCAACCACGACCTGTACTGCCAGGTGCACGGCAACGCC | |
| | CACGTGGCCAGCTGCGACGCCATCATGACCAGGTGCCTGGCCGTGCA | |
| | CGAGTGCTTCGTGAAGAGGGTGGACTGGACCATCGAGTACCCCATCAT | |
| | CGGCGACGAGCTGAAGATCAACGCCGCCTGCAGGAAGGTGCAGCACA | |
| | TGGTGGTGAAGGCCGCCCTGCTGGCCGACAAGTTCCCCGTGCTGCAC | |
| | GACATCGGCAACCCCAAGGCCATCAAGTGCGTGCCCCAGGCCGACGT | |
| | GGAGTGGAAGTTCTACGACGCCCAGCCCTGCAGCGACAAGGCCTACAA | |
| | GATCGAGGAGCTGTTCTACAGCTACGCCACCCACAGCGACAAGTTCAC | |
| | CGACGGCGTGTGCCTGTTCTGGAACTGCAACGTGGACAGGTACCCCG | |
| | CCAACAGCATCGTGTGCAGGTTCGACACCAGGGTGCTGAGCAACCTGA | |
| | ACCTGCCCGGCTGCGACGGCGGCAGCCTGTACGTGAACAAGCACGCC | |
| | TTCCACACCCCCGCCTTCGACAAGAGCGCCTTCGTGAACCTGAAGCAG | |
| | CTGCCCTTCTTCTACTACAGCGACAGCCCCTGCGAGAGCCACGGCAAG | |
| | CAGGTGGTGAGCGACATCGACTACGTGCCCCTGAAGAGCGCCACCTG | |
| | CATCACCAGGTGCAACCTGGGCGGCGCCGTGTGCAGGCACCACGCCA | |
| | ACGAGTACAGGCTGTACCTGGACGCCTACAACATGATGATCAGCGCCG | |
| | GCTTCAGCCTGTGGGTGTACAAGCAGTTCGACACCTACAACCTGTGGA | |
| | ACACCTTCACCAGGCTGCAGAGCCTGGAGAACGTGGCCTTCAACGTGG | |
| | TGAACAAGGGCCACTTCGACGGCCAGCAGGGCGAGGTGCCCGTGAGC | |
| | ATCATCAACAACACCGTGTACACCAAGGTGGACGGCGTGGACGTGGAG | |
| | CTGTTCGAGAACAAGACCACCCTGCCCGTGAACGTGGCCTTCGAGCTG | |
| | TGGGCCAAGAGGAACATCAAGCCCGTGCCCGAGGTGAAGATCCTGAAC | |
| | AACCTGGGCGTGGACATCGCCGCCAACACCGTGATCTGGGACTACAAG | |
| | AGGGACGCCCCCGCCCACATCAGCACCATCGGCGTGTGCAGCATGAC | |
| | CGACATCGCCAAGAAGCCCACCGAGACCATCTGCGCCCCCCTGACCGT | |
| | GTTCTTCGACGGCAGGGTGGACGGCCAGGTGGACCTGTTCAGGAACG | |
| | CCAGGAACGGCGTGCTGATCACCGAGGGCAGCGTGAAGGGCCTGCAG | |
| | CCCAGCGTGGGCCCCAAGCAGGCCAGCCTGAACGGCGTGACCCTGAT | |
| | CGGCGAGGCCGTGAAGACCCAGTTCAACTACTACAAGAAGGTGGACG | |
| | GCGTGGTGCAGCAGCTGCCCGAGACCTACTTCACCCAGAGCAGGAAC | |
| | CTGCAGGAGTTCAAGCCCAGGAGCCAGATGGAGATCGACTTCCTGGAG | |
| | CTGGCCATGGACGAGTTCATCGAGAGGTACAAGCTGGAGGGCTACGC | |
| | CTTCGAGCACATCGTGTACGGCGACTTCAGCCACAGCCAGCTGGGCG | |
| | GCCTGCACCTGCTGATCGGCCTGGCCAAGAGGTTCAAGGAGAGCCCC | |
| | TTCGAGCTGGAGGACTTCATCCCCATGGACAGCACCGTGAAGAACTAC | |
| | TTCATCACCGACGCCCAGACCGGCAGCAGCAAGTGCGTGTGCAGCGT | |
| | GATCGACCTGCTGCTGGACGACTTCGTGGAGATCATCAAGAGCCAGGA | |
| | CCTGAGCGTGGTGAGCAAGGTGGTGAAGGTGACCATCGACTACACCGA | |
| | GATCAGCTTCATGCTGTGGTGCAAGGACGGCCACGTGGAGACCTTCTA | |
| | CCCCAAGCTGCAGAGCAGCCAGGCCTGGCAGCCCGGCGTGGCCATGC | |
| | CCAACCTGTACAAGATGCAGAGGATGCTGCTGGAGAAGTGCGACCTGC | |
| | AGAACTACGGCGACAGCGCCACCCTGCCCAAGGGCATCATGATGAACG | |
| | TGGCCAAGTACACCCAGCTGTGCCAGTACCTGAACACCCTGACCCTGG | |
| | CCGTGCCCTACAACATGAGGGTGATCCACTTCGGCGCCGGCAGCGAC | |
| | AAGGGCGTGGCCCCCGGCACCGCCGTGCTGAGGCAGTGGCTGCCCAC | |
| | CGGCACCCTGCTGGTGGACAGCGACCTGAACGACTTCGTGAGCGACG | |
| | CCGACAGCACCCTGATCGGCGACTGCGCCACCGTGCACACCGCCAAC | |
| | AAGTGGGACCTGATCATCAGCGACATGTACGACCCCAAGACCAAGAAC | |
| | GTGACCAAGGAGAACGACAGCAAGGAGGGCTTCTTCACCTACATCTGC | |
| | GGCTTCATCCAGCAGAAGCTGGCCCTGGGCGGCAGCGTGGCCATCAA | |
| | GATCACCGAGCACAGCTGGAACGCCGACCTGTACAAGCTGATGGGCCA | |
| | CTTCGCCTGGTGGACCGCCTTCGTGACCAACGTGAACGCCAGCAGCAG | |
| | CGAGGCCTTCCTGATCGGCTGCAACTACCTGGGCAAGCCCAGGGAGC | |
| | AGATCGACGGCTACGTGATGCACGCCAACTACATCTTCTGGAGGAACA | |
| | CCAACCCCATCCAGCTGAGCAGCTACAGCCTGTTCGACATGAGCAAGT | |
| | TCCCCCTGAAGCTTGATGA | |
| 57 | ATGTTCGTGTTCCTGGTGCTGCTGCCCCTGGTGAGCAGCCAGTGCGTG | 342 |
| Pan-CoV (B5) | AACCTGACCACCAGGACCCAGCTGCCCCCCGCCTACACCAACAGCTTC | |
| | ACCAGGGGCGTGTACTACCCCGACAAGGTGTTCAGGAGCAGCGTGCT | |
| | GCACAGCACCCAGGACCTGTTCCTGCCCTTCTTCAGCAACGTGACCTG | |
| | GTTCCACGCCATCCACGTGAGCGGCACCAACGGCACCAAGAGGTTCGA | |
| | CAACCCCGTGCTGCCCTTCAACGACGGCGTGTACTTCGCCAGCACCGA | |
| | GAAGAGCAACATCATCAGGGGCTGGATCTTCGGCACCACCCTGGACAG | |
| | CAAGACCCAGAGCCTGCTGATCGTGAACAACGCCACCAACGTGGTGAT | |
| | CAAGGTGTGCGAGTTCCAGTTCTGCAACGACCCCTTCCTGGGCGTGTA | |
| | CTACCACAAGAACAACAAGAGCTGGATGGAGAGCGAGTTCAGGGTGTA | |
| | CAGCAGCGCCAACAACTGCACCTTCGAGTACGTGAGCCAGCCCTTCCT | |
| | GATGGACCTGGAGGGCAAGCAGGGCAACTTCAAGAACCTGAGGGAGT | |
| | TCGTGTTCAAGAACATCGACGGCTACTTCAAGATCTACAGCAAGCACAC | |
| | CCCCATCAACCTGGTGAGGGACCTGCCCCAGGGCTTCAGCGCCCTGG | |
| | AGCCCCTGGTGGACCTGCCCATCGGCATCAACATCACCAGGTTCCAGA | |
| | CCCTGCTGGCCCTGCACAGGAGCTACCTGACCCCCGGCGACAGCAGC | |
| | AGCGGCTGGACCGCCGGCGCCGCCGCCTACTACGTGGGCTACCTGCA | |
| | GCCCAGGACCTTCCTGCTGAAGTACAACGAGAACGGCACCATCACCGA | |
| | CGCCGTGGACTGCGCCCTGGACCCCCTGAGCGAGACCAAGTGCACCC | |
| | TGAAGAGCTTCACCGTGGAGAAGGGCATCTACCAGACCAGCAACTTCA | |
| | GGGTGCAGCCCACCGAGAGCATCGTGAGGTTCCCCAACATCACCAACC | |
| | TGTGCCCCTTCGGCGAGGTGTTCAACGCCACCAGGTTCGCCAGCGTGT | |
| | ACGCCTGGAACAGGAAGAGGATCAGCAACTGCGTGGCCGACTACAGC | |
| | GTGCTGTACAACAGCGCCAGCTTCAGCACCTTCAAGTGCTACGGCGTG | |
| | AGCCCCACCAAGCTGAACGACCTGTGCTTCACCAACGTGTACGCCGAC | |
| | AGCTTCGTGATCAGGGGCGACGAGGTGAGGCAGATCGCCCCCGGCCA | |
| | GACCGGCAAGATCGCCGACTACAACTACAAGCTGCCCGACGACTTCAC | |
| | CGGCTGCGTGATCGCCTGGAACAGCAACAACCTGGACAGCAAGGTGG | |
| | GCGGCAACTACAACTACCTGTACAGGCTGTTCAGGAAGAGCAACCTGA | |
| | AGCCCTTCGAGAGGGACATCAGCACCGAGATCTACCAGGCCGGCAGC | |
| | ACCCCCTGCAACGGCGTGGAGGGCTTCAACTGCTACTTCCCCCTGCAG | |
| | AGCTACGGCTTCCAGCCCACCAACGGCGTGGGCTACCAGCCCTACAG | |
| | GGTGGTGGTGCTGAGCTTCGAGCTGCTGCACGCCCCCGCCACCGTGT | |
| | GCGGCCCCAAGAAGAGCACCAACCTGGTGAAGAACAAGTGCGTGAACT | |
| | TCAACTTCAACGGCCTGACCGGCACCGGCGTGCTGACCGAGAGCAACA | |
| | AGAAGTTCCTGCCCTTCCAGCAGTTCGGCAGGGACATCGCCGACACCA | |
| | CCGACGCCGTGAGGGACCCCCAGACCCTGGAGATCCTGGACATCACC | |
| | CCCTGCAGCTTCGGCGGCGTGAGCGTGATCACCCCCGGCACCAACAC | |
| | CAGCAACCAGGTGGCCGTGCTGTACCAGGACGTGAACTGCACCGAGG | |
| | TGCCCGTGGCCATCCACGCCGACCAGCTGACCCCCACCTGGAGGGTG | |
| | TACAGCACCGGCAGCAACGTGTTCCAGACCAGGGCCGGCTGCCTGAT | |
| | CGGCGCCGAGCACGTGAACAACAGCTACGAGTGCGACATCCCCATCG | |
| | GCGCCGGCATCTGCGCCAGCTACCAGACCCAGACCAACAGCCCCGGC | |
| | AGCGCCAGCAGCGTGGCCAGCCAGAGCATCATCGCCTACACCATGAG | |
| | CCTGGGCGCCGAGAACAGCGTGGCCTACAGCAACAACAGCATCGCCA | |
| | TCCCCACCAACTTCACCATCAGCGTGACCACCGAGATCCTGCCCGTGA | |
| | GCATGACCAAGACCAGCGTGGACTGCACCATGTACATCTGCGGCGACA | |
| | GCACCGAGTGCAGCAACCTGCTGCTGCAGTACGGCAGCTTCTGCACCC | |
| | AGCTGAACAGGGCCCTGACCGGCATCGCCGTGGAGCAGGACAAGAAC | |
| | ACCCAGGAGGTGTTCGCCCAGGTGAAGCAGATCTACAAGACCCCCCCC | |
| | ATCAAGGACTTCGGCGGCTTCAACTTCAGCCAGATCCTGCCCGACCCC | |
| | AGCAAGCCCAGCAAGAGGAGCCCCATCGAGGACCTGCTGTTCAACAAG | |
| | GTGACCCTGGCCGACGCCGGCTTCATCAAGCAGTACGGCGACTGCCT | |
| | GGGCGACATCGCCGCCAGGGACCTGATCTGCGCCCAGAAGTTCAACG | |
| | GCCTGACCGTGCTGCCCCCCCTGCTGACCGACGAGATGATCGCCCAG | |
| | TACACCAGCGCCCTGCTGGCCGGCACCATCACCAGCGGCTGGACCTT | |
| | CGGCGCCGGCCCCGCCCTGCAGATCCCCTTCCCCATGCAGATGGCCT | |
| | ACAGGTTCAACGGCATCGGCGTGACCCAGAACGTGCTGTACGAGAACC | |
| | AGAAGCTGATCGCCAACCAGTTCAACAGCGCCATCGGCAAGATCCAGG | |
| | ACAGCCTGAGCAGCACCCCCAGCGCCCTGGGCAAGCTGCAGGACGTG | |
| | GTGAACCAGAACGCCCAGGCCCTGAACACCCTGGTGAAGCAGCTGAG | |
| | CAGCAACTTCGGCGCCATCAGCAGCGTGCTGAACGACATCCTGAGCAG | |
| | GCTGGACCCCCCCGAGGCCGAGGTGCAGATCGACAGGCTGATCACCG | |
| | GCAGGCTGCAGAGCCTGCAGACCTACGTGACCCAGCAGCTGATCAGG | |
| | GCCGCCGAGATCAGGGCCAGCGCCAACCTGGCCGCCACCAAGATGAG | |
| | CGAGTGCGTGCTGGGCCAGAGCAAGAGGGTGGACTTCTGCGGCAAGG | |
| | GCTACCACCTGATGAGCTTCCCCCAGAGCGCCCCCCACGGCGTGGTG | |
| | TTCCTGCACGTGACCTACGTGCCCGCCCAGGAGAAGAACTTCACCACC | |
| | GCCCCCGCCATCTGCCACGACGGCAAGGCCCACTTCCCCAGGGAGGG | |
| | CGTGTTCGTGAGCAACGGCACCCACTGGTTCGTGACCCAGAGGAACTT | |
| | CTACGAGCCCCAGATCATCACCACCGACAACACCTTCGTGAGCGGCAA | |
| | CTGCGACGTGGTGATCGGCATCGTGAACAACACCGTGTACGACCCCCT | |
| | GCAGCCCGAGCTGGACAGCTTCAAGGAGGAGCTGGACAAGTACTTCAA | |
| | GAACCACACCAGCCCCGACGTGGACCTGGGCGACATCAGCGGCATCA | |
| | ACGCCAGCGTGGTGAACATCCAGAAGGAGATCGACAGGCTGAACGAG | |
| | GTGGCCAAGAACCTGAACGAGAGCCTGATCGACCTGCAGGAGCTGGG | |
| | CAAGTACGAGCAGTACATCAAGTGGCCCTGGTACATCTGGCTGGGCTT | |
| | CATCGCCGGCCTGATCGCCATCGTGATGGTGACCATCATGCTGTGCTG | |
| | CATGACCAGCTGCTGCAGCTGCCTGAAGGGCTGCTGCAGCTGCGGCA | |
| | GCTGCTGCAAGTTCGACGAGGACGACAGCGAGCCCGTGCTGAAGGGC | |
| | GTGAAGCTGCACTACACCGGAAGCGGAGCCACGAACTTCTCTCTGTTA | |
| | AAGCAAGCAGGAGATGTTGAAGAAAACCCCGGGCCTATGTACAGCTTC | |
| | GTGAGCGAGGAGACCGGCACCCTGATCGTGAACAGCGTGCTGCTGTT | |
| | CCTGGCCTTCGTGGTGTTCCTGCTGGTGACCCTGGCCATCCTGACCGC | |
| | CCTGAGGCTGTGCGCCTACTGCTGCAACATCGTGAACGTGAGCCTGGT | |
| | GAAGCCCAGCTTCTACGTGTACAGCAGGGTGAAGAACCTGAACAGCAG | |
| | CAGGGTGCCCGACCTGCTGGTGGGAAGCGGAGCCACGAACTTCTCTC | |
| | TGTTAAAGCAAGCAGGAGATGTTGAAGAAAACCCCGGGCCTATGGCCG | |
| | ACAGCAACGGCACCATCACCGTGGAGGAGCTGAAGAAGCTGCTGGAG | |
| | CAGTGGAACCTGGTGATCGGCTTCCTGTTCCTGACCTGGATCTGCCTG | |
| | CTGCAGTTCGCCTACGCCAACAGGAACAGGTTCCTGTACATCATCAAG | |
| | CTGATCTTCCTGTGGCTGCTGTGGCCCGTGACCCTGGCCTGCTTCGTG | |
| | CTGGCCGCCGTGTACAGGATCAACTGGATCACCGGCGGCATCGCCATC | |
| | GCCATGGCCTGCCTGGTGGGCCTGATGTGGCTGAGCTACTTCATCGCC | |
| | AGCTTCAGGCTGTTCGCCAGGACCAGGAGCATGTGGAGCTTCAACCCC | |
| | GAGACCAACATCCTGCTGAACGTGCCCCTGCACGGCACCATCCTGACC | |
| | AGGCCCCTGCTGGAGAGCGAGCTGGTGATCGGCGCCGTGATCCTGAG | |
| | GGGCCACCTGAGGATCGCCGGCCACCACCTGGGCAGGTGCGACATCA | |
| | AGGACCTGCCCAAGGAGATCACCGTGGCCACCAGCAGGACCCTGAGC | |
| | TACTACAAGCTGGGCGCCAGCCAGAGGGTGGCCGGCGACAGCGGCTT | |
| | CGCCGCCTACAGCAGGTACAGGATCGGCAACTACAAGCTGAACACCGA | |
| | CCACAGCAGCAGCAGCGACAACATCGCCCTGCTGGTGCAGGGAAGCG | |
| | GAGCCACGAACTTCTCTCTGTTAAAGCAAGCAGGAGATGTTGAAGAAAA | |
| | CCCCGGGCCTATGAAGATCATCCTGTTCCTGGCCCTGATCACCCTGGC | |
| | CACCTGCGAGCTGTACCACTACCAGGAGTGCGTGAGGGGCACCACCG | |
| | TGTGATGA | |
| 58 | ATGTTCGTGTTCCTGGTGCTGCTGCCCCTGGTGAGCAGCCAGTGCGTG | 343 |
| Pan-CoV (B6) | AACCTGACCACCAGGACCCAGCTGCCCCCCGCCTACACCAACAGCTTC | |
| | ACCAGGGGCGTGTACTACCCCGACAAGGTGTTCAGGAGCAGCGTGCT | |
| | GCACAGCACCCAGGACCTGTTCCTGCCCTTCTTCAGCAACGTGACCTG | |
| | GTTCCACGCCATCCACGTGAGCGGCACCAACGGCACCAAGAGGTTCGA | |
| | CAACCCCGTGCTGCCCTTCAACGACGGCGTGTACTTCGCCAGCACCGA | |
| | GAAGAGCAACATCATCAGGGGCTGGATCTTCGGCACCACCCTGGACAG | |
| | CAAGACCCAGAGCCTGCTGATCGTGAACAACGCCACCAACGTGGTGAT | |
| | CAAGGTGTGCGAGTTCCAGTTCTGCAACGACCCCTTCCTGGGCGTGTA | |
| | CTACCACAAGAACAACAAGAGCTGGATGGAGAGCGAGTTCAGGGTGTA | |
| | CAGCAGCGCCAACAACTGCACCTTCGAGTACGTGAGCCAGCCCTTCCT | |
| | GATGGACCTGGAGGGCAAGCAGGGCAACTTCAAGAACCTGAGGGAGT | |
| | TCGTGTTCAAGAACATCGACGGCTACTTCAAGATCTACAGCAAGCACAC | |
| | CCCCATCAACCTGGTGAGGGACCTGCCCCAGGGCTTCAGCGCCCTGG | |
| | AGCCCCTGGTGGACCTGCCCATCGGCATCAACATCACCAGGTTCCAGA | |
| | CCCTGCTGGCCCTGCACAGGAGCTACCTGACCCCCGGCGACAGCAGC | |
| | AGCGGCTGGACCGCCGGCGCCGCCGCCTACTACGTGGGCTACCTGCA | |
| | GCCCAGGACCTTCCTGCTGAAGTACAACGAGAACGGCACCATCACCGA | |
| | CGCCGTGGACTGCGCCCTGGACCCCCTGAGCGAGACCAAGTGCACCC | |
| | TGAAGAGCTTCACCGTGGAGAAGGGCATCTACCAGACCAGCAACTTCA | |
| | GGGTGCAGCCCACCGAGAGCATCGTGAGGTTCCCCAACATCACCAACC | |
| | TGTGCCCCTTCGGCGAGGTGTTCAACGCCACCAGGTTCGCCAGCGTGT | |
| | ACGCCTGGAACAGGAAGAGGATCAGCAACTGCGTGGCCGACTACAGC | |
| | GTGCTGTACAACAGCGCCAGCTTCAGCACCTTCAAGTGCTACGGCGTG | |
| | AGCCCCACCAAGCTGAACGACCTGTGCTTCACCAACGTGTACGCCGAC | |
| | AGCTTCGTGATCAGGGGCGACGAGGTGAGGCAGATCGCCCCCGGCCA | |
| | GACCGGCAAGATCGCCGACTACAACTACAAGCTGCCCGACGACTTCAC | |
| | CGGCTGCGTGATCGCCTGGAACAGCAACAACCTGGACAGCAAGGTGG | |
| | GCGGCAACTACAACTACCTGTACAGGCTGTTCAGGAAGAGCAACCTGA | |
| | AGCCCTTCGAGAGGGACATCAGCACCGAGATCTACCAGGCCGGCAGC | |
| | ACCCCCTGCAACGGCGTGGAGGGCTTCAACTGCTACTTCCCCCTGCAG | |
| | AGCTACGGCTTCCAGCCCACCAACGGCGTGGGCTACCAGCCCTACAG | |
| | GGTGGTGGTGCTGAGCTTCGAGCTGCTGCACGCCCCCGCCACCGTGT | |
| | GCGGCCCCAAGAAGAGCACCAACCTGGTGAAGAACAAGTGCGTGAACT | |
| | TCAACTTCAACGGCCTGACCGGCACCGGCGTGCTGACCGAGAGCAACA | |
| | AGAAGTTCCTGCCCTTCCAGCAGTTCGGCAGGGACATCGCCGACACCA | |
| | CCGACGCCGTGAGGGACCCCCAGACCCTGGAGATCCTGGACATCACC | |
| | CCCTGCAGCTTCGGCGGCGTGAGCGTGATCACCCCCGGCACCAACAC | |
| | CAGCAACCAGGTGGCCGTGCTGTACCAGGACGTGAACTGCACCGAGG | |
| | TGCCCGTGGCCATCCACGCCGACCAGCTGACCCCCACCTGGAGGGTG | |
| | TACAGCACCGGCAGCAACGTGTTCCAGACCAGGGCCGGCTGCCTGAT | |
| | CGGCGCCGAGCACGTGAACAACAGCTACGAGTGCGACATCCCCATCG | |
| | GCGCCGGCATCTGCGCCAGCTACCAGACCCAGACCAACAGCCCCGGC | |
| | AGCGCCAGCAGCGTGGCCAGCCAGAGCATCATCGCCTACACCATGAG | |
| | CCTGGGCGCCGAGAACAGCGTGGCCTACAGCAACAACAGCATCGCCA | |
| | TCCCCACCAACTTCACCATCAGCGTGACCACCGAGATCCTGCCCGTGA | |
| | GCATGACCAAGACCAGCGTGGACTGCACCATGTACATCTGCGGCGACA | |
| | GCACCGAGTGCAGCAACCTGCTGCTGCAGTACGGCAGCTTCTGCACCC | |
| | AGCTGAACAGGGCCCTGACCGGCATCGCCGTGGAGCAGGACAAGAAC | |
| | ACCCAGGAGGTGTTCGCCCAGGTGAAGCAGATCTACAAGACCCCCCCC | |
| | ATCAAGGACTTCGGCGGCTTCAACTTCAGCCAGATCCTGCCCGACCCC | |
| | AGCAAGCCCAGCAAGAGGAGCCCCATCGAGGACCTGCTGTTCAACAAG | |
| | GTGACCCTGGCCGACGCCGGCTTCATCAAGCAGTACGGCGACTGCCT | |
| | GGGCGACATCGCCGCCAGGGACCTGATCTGCGCCCAGAAGTTCAACG | |
| | GCCTGACCGTGCTGCCCCCCCTGCTGACCGACGAGATGATCGCCCAG | |
| | TACACCAGCGCCCTGCTGGCCGGCACCATCACCAGCGGCTGGACCTT | |
| | CGGCGCCGGCCCCGCCCTGCAGATCCCCTTCCCCATGCAGATGGCCT | |
| | ACAGGTTCAACGGCATCGGCGTGACCCAGAACGTGCTGTACGAGAACC | |
| | AGAAGCTGATCGCCAACCAGTTCAACAGCGCCATCGGCAAGATCCAGG | |
| | ACAGCCTGAGCAGCACCCCCAGCGCCCTGGGCAAGCTGCAGGACGTG | |
| | GTGAACCAGAACGCCCAGGCCCTGAACACCCTGGTGAAGCAGCTGAG | |
| | CAGCAACTTCGGCGCCATCAGCAGCGTGCTGAACGACATCCTGAGCAG | |
| | GCTGGACCCCCCCGAGGCCGAGGTGCAGATCGACAGGCTGATCACCG | |
| | GCAGGCTGCAGAGCCTGCAGACCTACGTGACCCAGCAGCTGATCAGG | |
| | GCCGCCGAGATCAGGGCCAGCGCCAACCTGGCCGCCACCAAGATGAG | |
| | CGAGTGCGTGCTGGGCCAGAGCAAGAGGGTGGACTTCTGCGGCAAGG | |
| | GCTACCACCTGATGAGCTTCCCCCAGAGCGCCCCCCACGGCGTGGTG | |
| | TTCCTGCACGTGACCTACGTGCCCGCCCAGGAGAAGAACTTCACCACC | |
| | GCCCCCGCCATCTGCCACGACGGCAAGGCCCACTTCCCCAGGGAGGG | |
| | CGTGTTCGTGAGCAACGGCACCCACTGGTTCGTGACCCAGAGGAACTT | |
| | CTACGAGCCCCAGATCATCACCACCGACAACACCTTCGTGAGCGGCAA | |
| | CTGCGACGTGGTGATCGGCATCGTGAACAACACCGTGTACGACCCCCT | |
| | GCAGCCCGAGCTGGACAGCTTCAAGGAGGAGCTGGACAAGTACTTCAA | |
| | GAACCACACCAGCCCCGACGTGGACCTGGGCGACATCAGCGGCATCA | |
| | ACGCCAGCGTGGTGAACATCCAGAAGGAGATCGACAGGCTGAACGAG | |
| | GTGGCCAAGAACCTGAACGAGAGCCTGATCGACCTGCAGGAGCTGGG | |
| | CAAGTACGAGCAGTACATCAAGTGGCCCTGGTACATCTGGCTGGGCTT | |
| | CATCGCCGGCCTGATCGCCATCGTGATGGTGACCATCATGCTGTGCTG | |
| | CATGACCAGCTGCTGCAGCTGCCTGAAGGGCTGCTGCAGCTGCGGCA | |
| | GCTGCTGCAAGTTCGACGAGGACGACAGCGAGCCCGTGCTGAAGGGC | |
| | GTGAAGCTGCACTACACCGGAAGCGGAGCCACGAACTTCTCTCTGTTA | |
| | AAGCAAGCAGGAGATGTTGAAGAAAACCCCGGGCCTGCCCCCACCAAG | |
| | GTGACCTTCGGCGACGACACCGTGATCGAGGTGCAGGGCTACAAGAG | |
| | CGTGAACATCACCTTCGAGCTGGACGAGAGGATCGACAAGGTGCTGAA | |
| | CGAGAAGTGCAGCGCCTACACCGTGGAGCTGGGCACCGAGGTGAACG | |
| | AGTTCGCCTGCGTGGTGGCCGACGCCGTGATCAAGACCCTGCAGCCC | |
| | GTGAGCGAGCTGCTGACCCCCCTGGGCATCGACCTGGACGAGTGGAG | |
| | CATGGCCACCTACTACCTGTTCGACGAGAGCGGCGAGTTCAAGCTGGC | |
| | CAGCCACATGTACTGCAGCTTCTACCCCCCCGACGAGGACGAGGAGGA | |
| | GGGCGACTGCGAGGAGGAGGAGTTCGAGCCCAGCACCCAGTACGAGT | |
| | ACGGCACCGAGGACGACTACCAGGGCAAGCCCCTGGAGTTCGGCGCC | |
| | ACCAGCGCCGCCCTGCAGCCCGAGGAGGAGCAGGAGGAGGACTGGC | |
| | TGGACGACGACAGCCAGCAGACCGTGGGCCAGCAGGACGGCAGCGA | |
| | GGACAACCAGACCACCACCATCCAGACCATCGTGGAGGTGCAGCCCCA | |
| | GCTGGAGATGGAGCTGACCCCCGTGGTGCAGACCATCGAGGTGAACA | |
| | GCTTCAGCGGCTACCTGAAGCTGACCGACAACGTGTACATCAAGAACG | |
| | CCGACATCGTGGAGGAGGCCAAGAAGGTGAAGCCCACCGTGGTGGTG | |
| | AACGCCGCCAACGTGTACCTGAAGCACGGCGGCGGCGTGGCCGGCGC | |
| | CCTGAACAAGGCCACCAACAACGCCATGCAGGTGGAGAGCGACGACTA | |
| | CATCGCCACCAACGGCCCCCTGAAGGTGGGCGGCAGCTGCGTGCTGA | |
| | GCGGCCACAACCTGGCCAAGCACTGCCTGCACGTGGTGGGCCCCAAC | |
| | GTGAACAAGGGCGAGGACATCCAGCTGCTGAAGAGCGCCTACGAGAA | |
| | CTTCAACCAGCACGAGGTGCTGCTGGCCCCCCTGCTGAGCGCCGGCA | |
| | TCTTCGGCGCCGACCCCATCCACAGCCTGAGGGTGTGCGTGGACACC | |
| | GTGAGGACCAACGTGTACCTGGCCGTGTTCGACAAGAACCTGTACGAC | |
| | AAGCTGGTGAGCAGCTTCCTGGAGATGAAGAGCGAGAAGCAGGTGGA | |
| | GCAGAAGATCGCCGAGATCCCCAAGGAGGAGGTGAAGCCCTTCATCAC | |
| | CGAGAGCAAGCCCAGCGTGGAGCAGAGGAAGCAGGACGACAAGAAGA | |
| | TCAAGGCCTGCGTGGAGGAGGTGACCACCACCCTGGAGGAGACCAAG | |
| | TTCCTGACCGAGAACCTGCTGCTGTACATCGACATCAACGGCAACCTG | |
| | CACCCCGACAGCGCCACCCTGGTGAGCGACATCGACATCACCTTCCTG | |
| | AAGAAGGACGCCCCCTACATCGTGGGCGACGTGGTGCAGGAGGGCGT | |
| | GCTGACCGCCGTGGTGATCCCCACCAAGAAGGCCGGCGGCACCACCG | |
| | AGATGCTGGCCAAGGCCCTGAGGAAGGTGCCCACCGACAACTACATCA | |
| | CCACCTACCCCGGCCAGGGCCTGAACGGCTACACCGTGGAGGAGGCC | |
| | AAGACCGTGCTGAAGAAGTGCAAGAGCGCCTTCTACATCCTGCCCAGC | |
| | ATCATCAGCAACGAGAAGCAGGAGATCCTGGGCACCGTGAGCTGGAAC | |
| | CTGAGGGAGATGCTGGCCCACGCCGAGGAGACCAGGAAGCTGATGCC | |
| | CGTGTGCGTGGAGACCAAGGCCATCGTGAGCACCATCCAGAGGAAGTA | |
| | CAAGGGCATCAAGATCCAGGAGGGCGTGGTGGACTACGGCGCCAGGT | |
| | TCTACTTCTACACCAGCAAGACCACCGTGGCCAGCCTGATCAACACCCT | |
| | GAACGACCTGAACGAGACCCTGGTGACCATGCCCCTGGGCTACGTGAC | |
| | CCACGGCCTGAACCTGGAGGAGGCCGCCAGGTACATGAGGAGCCTGA | |
| | AGGTGCCCGCCACCGTGAGCGTGAGCAGCCCCGACGCCGTGACCGCC | |
| | TACAACGGCTACCTGACCAGCAGCAGCAAGACCCCCGAGGAGCACTTC | |
| | ATCGAGACCATCAGCCTGGCCGGCAGCTACAAGGACTGGAGCTACAGC | |
| | GGCCAGAGCACCCAGCTGGGCATCGAGTTCCTGAAGAGGGGCGACAA | |
| | GAGCGTGTACTACACCAGCAACCCCACCACCTTCCACCTGGACGGCGA | |
| | GGTGATCACCTTCGACAACCTGAAGACCCTGCTGAGCCTGAGGGAGGT | |
| | GAGGACCATCAAGGTGTTCACCACCGTGGACAACATCAACCTGCACGG | |
| | AAGCGGAGCCACGAACTTCTCTCTGTTAAAGCAAGCAGGAGATGTTGA | |
| | AGAAAACCCCGGGCCTAGCGGCTTCAGGAAGATGGCCTTCCCCAGCG | |
| | GCAAGGTGGAGGGCTGCATGGTGCAGGTGACCTGCGGCACCACCACC | |
| | CTGAACGGCCTGTGGCTGGACGACGTGGTGTACTGCCCCAGGCACGT | |
| | GATCTGCACCAGCGAGGACATGCTGAACCCCAACTACGAGGACCTGCT | |
| | GATCAGGAAGAGCAACCACAACTTCCTGGTGCAGGCCGGCAACGTGCA | |
| | GCTGAGGGTGATCGGCCACAGCATGCAGAACTGCGTGCTGAAGCTGAA | |
| | GGTGGACACCGCCAACCCCAAGACCCCCAAGTACAAGTTCGTGAGGAT | |
| | CCAGCCCGGCCAGACCTTCAGCGTGCTGGCCTGCTACAACGGCAGCC | |
| | CCAGCGGCGTGTACCAGTGCGCCATGAGGCCCAACTTCACCATCAAGG | |
| | GCAGCTTCCTGAACGGCAGCTGCGGCAGCGTGGGCTTCAACATCGACT | |
| | ACGACTGCGTGAGCTTCTGCTACATGCACCACATGGAGCTGCCCACCG | |
| | GCGTGCACGCCGGCACCGACCTGGAGGGCAACTTCTACGGCCCCTTC | |
| | GTGGACAGGCAGACCGCCCAGGCCGCCGGCACCGACACCACCATCAC | |
| | CGTGAACGTGCTGGCCTGGCTGTACGCCGCCGTGATCAACGGCGACA | |
| | GGTGGTTCCTGAACAGGTTCACCACCACCCTGAACGACTTCAACCTGG | |
| | TGGCCATGAAGTACAACTACGAGCCCCTGACCCAGGACCACGTGGACA | |
| | TCCTGGGCCCCCTGAGCGCCCAGACCGGCATCGCCGTGCTGGACATG | |
| | TGCGCCAGCCTGAAGGAGCTGCTGCAGAACGGCATGAACGGCAGGAC | |
| | CATCCTGGGCAGCGCCCTGCTGGAGGACGAGTTCACCCCCTTCGACGT | |
| | GGTGAGGCAGTGCAGCGGCGTGACCTTCCAGGGAAGCGGAGCCACGA | |
| | ACTTCTCTCTGTTAAAGCAAGCAGGAGATGTTGAAGAAAACCCCGGGC | |
| | CTAGCGCCGACGCCCAGAGCTTCCTGAACAGGGTGTGCGGCGTGAGC | |
| | GCCGCCAGGCTGACCCCCTGCGGCACCGGCACCAGCACCGACGTGGT | |
| | GTACAGGGCCTTCGACATCTACAACGACAAGGTGGCCGGCTTCGCCAA | |
| | GTTCCTGAAGACCAACTGCTGCAGGTTCCAGGAGAAGGACGAGGACGA | |
| | CAACCTGATCGACAGCTACTTCGTGGTGAAGAGGCACACCTTCAGCAA | |
| | CTACCAGCACGAGGAGACCATCTACAACCTGCTGAAGGACTGCCCCGC | |
| | CGTGGCCAAGCACGACTTCTTCAAGTTCAGGATCGACGGCGACATGGT | |
| | GCCCCACATCAGCAGGCAGAGGCTGACCAAGTACACCATGGCCGACCT | |
| | GGTGTACGCCCTGAGGCACTTCGACGAGGGCAACTGCGACACCCTGA | |
| | AGGAGATCCTGGTGACCTACAACTGCTGCGACGACGACTACTTCAACA | |
| | AGAAGGACTGGTACGACTTCGTGGAGAACCCCGACATCCTGAGGGTGT | |
| | ACGCCAACCTGGGCGAGAGGGTGAGGCAGGCCCTGCTGAAGACCGTG | |
| | CAGTTCTGCGACGCCATGAGGAACGCCGGCATCGTGGGCGTGCTGAC | |
| | CCTGGACAACCAGGACCTGAACGGCAACTGGTACGACTTCGGCGACTT | |
| | CATCCAGACCACCCCCGGCAGCGGCGTGCCCGTGGTGGACAGCTACT | |
| | ACAGCCTGCTGATGCCCATCCTGACCCTGACCAGGGCCCTGACCGCC | |
| | GAGAGCCACGTGGACACCGACCTGACCAAGCCCTACATCAAGTGGGAC | |
| | CTGCTGAAGTACGACTTCACCGAGGAGAGGCTGAAGCTGTTCGACAGG | |
| | TACTTCAAGTACTGGGACCAGACCTACCACCCCAACTGCGTGAACTGC | |
| | CTGGACGACAGGTGCATCCTGCACTGCGCCAACTTCAACGTGCTGTTC | |
| | AGCACCGTGTTCCCCCCCACCAGCTTCGGCCCCCTGGTGAGGAAGATC | |
| | TTCGTGGACGGCGTGCCCTTCGTGGTGAGCACCGGCTACCACTTCAGG | |
| | GAGCTGGGCGTGGTGCACAACCAGGACGTGAACCTGCACAGCAGCAG | |
| | GCTGAGCTTCAAGGAGCTGCTGGTGTACGCCGCCGACCCCGCCATGC | |
| | ACGCCGCCAGCGGCAACCTGCTGCTGGACAAGAGGACCACCTGCTTC | |
| | AGCGTGGCCGCCCTGACCAACAACGTGGCCTTCCAGACCGTGAAGCC | |
| | CGGCAACTTCAACAAGGACTTCTACGACTTCGCCGTGAGCAAGGGCTT | |
| | CTTCAAGGAGGGCAGCAGCGTGGAGCTGAAGCACTTCTTCTTCGCCCA | |
| | GGACGGCAACGCCGCCATCAGCGACTACGACTACTACAGGTACAACCT | |
| | GCCCACCATGTGCGACATCAGGCAGCTGCTGTTCGTGGTGGAGGTGGT | |
| | GGACAAGTACTTCGACTGCTACGACGGCGGCTGCATCAACGCCAACCA | |
| | GGTGATCGTGAACAACCTGGACAAGAGCGCCGGCTTCCCCTTCAACAA | |
| | GTGGGGCAAGGCCAGGCTGTACTACGACAGCATGAGCTACGAGGACC | |
| | AGGACGCCCTGTTCGCCTACACCAAGAGGAACGTGATCCCCACCATCA | |
| | CCCAGATGAACCTGAAGTACGCCATCAGCGCCAAGAACAGGGCCAGGA | |
| | CCGTGGCCGGCGTGAGCATCTGCAGCACCATGACCAACAGGCAGTTC | |
| | CACCAGAAGCTGCTGAAGAGCATCGCCGCCACCAGGGGCGCCACCGT | |
| | GGTGATCGGCACCAGCAAGTTCTACGGCGGCTGGCACAACATGCTGAA | |
| | GACCGTGTACAGCGACGTGGAGAACCCCCACCTGATGGGCTGGGACT | |
| | ACCCCAAGTGCGACAGGGCCATGCCCAACATGCTGAGGATCATGGCCA | |
| | GCCTGGTGCTGGCCAGGAAGCACACCACCTGCTGCAGCCTGAGCCAC | |
| | AGGTTCTACAGGCTGGCCAACGAGTGCGCCCAGGTGCTGAGCGAGAT | |
| | GGTGATGTGCGGCGGCAGCCTGTACGTGAAGCCCGGCGGCACCAGCA | |
| | GCGGCGACGCCACCACCGCCTACGCCAACAGCGTGTTCAACATCTGCC | |
| | AGGCCGTGACCGCCAACGTGAACGCCCTGCTGAGCACCGACGGCAAC | |
| | AAGATCGCCGACAAGTACGTGAGGAACCTGCAGCACAGGCTGTACGAG | |
| | TGCCTGTACAGGAACAGGGACGTGGACACCGACTTCGTGAACGAGTTC | |
| | TACGCCTACCTGAGGAAGCACTTCAGCATGATGATCCTGAGCGACGAC | |
| | GCCGTGGTGTGCTTCAACAGCACCTACGCCAGCCAGGGCCTGGTGGC | |
| | CAGCATCAAGAACTTCAAGAGCGTGCTGTACTACCAGAACAACGTGTTC | |
| | ATGAGCGAGGCCAAGTGCTGGACCGAGACCGACCTGACCAAGGGCCC | |
| | CCACGAGTTCTGCAGCCAGCACACCATGCTGGTGAAGCAGGGCGACG | |
| | ACTACGTGTACCTGCCCTACCCCGACCCCAGCAGGATCCTGGGCGCC | |
| | GGCTGCTTCGTGGACGACATCGTGAAGACCGACGGCACCCTGATGATC | |
| | GAGAGGTTCGTGAGCCTGGCCATCGACGCCTACCCCCTGACCAAGCAC | |
| | CCCAACCAGGAGTACGCCGACGTGTTCCACCTGTACCTGCAGTACATC | |
| | AGGAAGCTGCACGACGAGCTGACCGGCCACATGCTGGACATGTACAG | |
| | CGTGATGCTGACCAACGACAACACCAGCAGGTACTGGGAGCCCGAGTT | |
| | CTACGAGGCCATGTACACCCCCCACACCGTGCTGCAGTGATGA | |
| 59 | ATGTTCGTGTTCCTGGTGCTGCTGCCCCTGGTGAGCAGCCAGTGCGTG | 344 |
| Pan-CoV | AACCTGACCACCAGGACCCAGCTGCCCCCCGCCTACACCAACAGCTTC | |
| (B6.1) | ACCAGGGGCGTGTACTACCCCGACAAGGTGTTCAGGAGCAGCGTGCT | |
| | GCACAGCACCCAGGACCTGTTCCTGCCCTTCTTCAGCAACGTGACCTG | |
| | GTTCCACGCCATCCACGTGAGCGGCACCAACGGCACCAAGAGGTTCGA | |
| | CAACCCCGTGCTGCCCTTCAACGACGGCGTGTACTTCGCCAGCACCGA | |
| | GAAGAGCAACATCATCAGGGGCTGGATCTTCGGCACCACCCTGGACAG | |
| | CAAGACCCAGAGCCTGCTGATCGTGAACAACGCCACCAACGTGGTGAT | |
| | CAAGGTGTGCGAGTTCCAGTTCTGCAACGACCCCTTCCTGGGCGTGTA | |
| | CTACCACAAGAACAACAAGAGCTGGATGGAGAGCGAGTTCAGGGTGTA | |
| | CAGCAGCGCCAACAACTGCACCTTCGAGTACGTGAGCCAGCCCTTCCT | |
| | GATGGACCTGGAGGGCAAGCAGGGCAACTTCAAGAACCTGAGGGAGT | |
| | TCGTGTTCAAGAACATCGACGGCTACTTCAAGATCTACAGCAAGCACAC | |
| | CCCCATCAACCTGGTGAGGGACCTGCCCCAGGGCTTCAGCGCCCTGG | |
| | AGCCCCTGGTGGACCTGCCCATCGGCATCAACATCACCAGGTTCCAGA | |
| | CCCTGCTGGCCCTGCACAGGAGCTACCTGACCCCCGGCGACAGCAGC | |
| | AGCGGCTGGACCGCCGGCGCCGCCGCCTACTACGTGGGCTACCTGCA | |
| | GCCCAGGACCTTCCTGCTGAAGTACAACGAGAACGGCACCATCACCGA | |
| | CGCCGTGGACTGCGCCCTGGACCCCCTGAGCGAGACCAAGTGCACCC | |
| | TGAAGAGCTTCACCGTGGAGAAGGGCATCTACCAGACCAGCAACTTCA | |
| | GGGTGCAGCCCACCGAGAGCATCGTGAGGTTCCCCAACATCACCAACC | |
| | TGTGCCCCTTCGGCGAGGTGTTCAACGCCACCAGGTTCGCCAGCGTGT | |
| | ACGCCTGGAACAGGAAGAGGATCAGCAACTGCGTGGCCGACTACAGC | |
| | GTGCTGTACAACAGCGCCAGCTTCAGCACCTTCAAGTGCTACGGCGTG | |
| | AGCCCCACCAAGCTGAACGACCTGTGCTTCACCAACGTGTACGCCGAC | |
| | AGCTTCGTGATCAGGGGCGACGAGGTGAGGCAGATCGCCCCCGGCCA | |
| | GACCGGCAAGATCGCCGACTACAACTACAAGCTGCCCGACGACTTCAC | |
| | CGGCTGCGTGATCGCCTGGAACAGCAACAACCTGGACAGCAAGGTGG | |
| | GCGGCAACTACAACTACCTGTACAGGCTGTTCAGGAAGAGCAACCTGA | |
| | AGCCCTTCGAGAGGGACATCAGCACCGAGATCTACCAGGCCGGCAGC | |
| | ACCCCCTGCAACGGCGTGGAGGGCTTCAACTGCTACTTCCCCCTGCAG | |
| | AGCTACGGCTTCCAGCCCACCAACGGCGTGGGCTACCAGCCCTACAG | |
| | GGTGGTGGTGCTGAGCTTCGAGCTGCTGCACGCCCCCGCCACCGTGT | |
| | GCGGCCCCAAGAAGAGCACCAACCTGGTGAAGAACAAGTGCGTGAACT | |
| | TCAACTTCAACGGCCTGACCGGCACCGGCGTGCTGACCGAGAGCAACA | |
| | AGAAGTTCCTGCCCTTCCAGCAGTTCGGCAGGGACATCGCCGACACCA | |
| | CCGACGCCGTGAGGGACCCCCAGACCCTGGAGATCCTGGACATCACC | |
| | CCCTGCAGCTTCGGCGGCGTGAGCGTGATCACCCCCGGCACCAACAC | |
| | CAGCAACCAGGTGGCCGTGCTGTACCAGGACGTGAACTGCACCGAGG | |
| | TGCCCGTGGCCATCCACGCCGACCAGCTGACCCCCACCTGGAGGGTG | |
| | TACAGCACCGGCAGCAACGTGTTCCAGACCAGGGCCGGCTGCCTGAT | |
| | CGGCGCCGAGCACGTGAACAACAGCTACGAGTGCGACATCCCCATCG | |
| | GCGCCGGCATCTGCGCCAGCTACCAGACCCAGACCAACAGCCCCGGC | |
| | AGCGCCAGCAGCGTGGCCAGCCAGAGCATCATCGCCTACACCATGAG | |
| | CCTGGGCGCCGAGAACAGCGTGGCCTACAGCAACAACAGCATCGCCA | |
| | TCCCCACCAACTTCACCATCAGCGTGACCACCGAGATCCTGCCCGTGA | |
| | GCATGACCAAGACCAGCGTGGACTGCACCATGTACATCTGCGGCGACA | |
| | GCACCGAGTGCAGCAACCTGCTGCTGCAGTACGGCAGCTTCTGCACCC | |
| | AGCTGAACAGGGCCCTGACCGGCATCGCCGTGGAGCAGGACAAGAAC | |
| | ACCCAGGAGGTGTTCGCCCAGGTGAAGCAGATCTACAAGACCCCCCCC | |
| | ATCAAGGACTTCGGCGGCTTCAACTTCAGCCAGATCCTGCCCGACCCC | |
| | AGCAAGCCCAGCAAGAGGAGCCCCATCGAGGACCTGCTGTTCAACAAG | |
| | GTGACCCTGGCCGACGCCGGCTTCATCAAGCAGTACGGCGACTGCCT | |
| | GGGCGACATCGCCGCCAGGGACCTGATCTGCGCCCAGAAGTTCAACG | |
| | GCCTGACCGTGCTGCCCCCCCTGCTGACCGACGAGATGATCGCCCAG | |
| | TACACCAGCGCCCTGCTGGCCGGCACCATCACCAGCGGCTGGACCTT | |
| | CGGCGCCGGCCCCGCCCTGCAGATCCCCTTCCCCATGCAGATGGCCT | |
| | ACAGGTTCAACGGCATCGGCGTGACCCAGAACGTGCTGTACGAGAACC | |
| | AGAAGCTGATCGCCAACCAGTTCAACAGCGCCATCGGCAAGATCCAGG | |
| | ACAGCCTGAGCAGCACCCCCAGCGCCCTGGGCAAGCTGCAGGACGTG | |
| | GTGAACCAGAACGCCCAGGCCCTGAACACCCTGGTGAAGCAGCTGAG | |
| | CAGCAACTTCGGCGCCATCAGCAGCGTGCTGAACGACATCCTGAGCAG | |
| | GCTGGACCCCCCCGAGGCCGAGGTGCAGATCGACAGGCTGATCACCG | |
| | GCAGGCTGCAGAGCCTGCAGACCTACGTGACCCAGCAGCTGATCAGG | |
| | GCCGCCGAGATCAGGGCCAGCGCCAACCTGGCCGCCACCAAGATGAG | |
| | CGAGTGCGTGCTGGGCCAGAGCAAGAGGGTGGACTTCTGCGGCAAGG | |
| | GCTACCACCTGATGAGCTTCCCCCAGAGCGCCCCCCACGGCGTGGTG | |
| | TTCCTGCACGTGACCTACGTGCCCGCCCAGGAGAAGAACTTCACCACC | |
| | GCCCCCGCCATCTGCCACGACGGCAAGGCCCACTTCCCCAGGGAGGG | |
| | CGTGTTCGTGAGCAACGGCACCCACTGGTTCGTGACCCAGAGGAACTT | |
| | CTACGAGCCCCAGATCATCACCACCGACAACACCTTCGTGAGCGGCAA | |
| | CTGCGACGTGGTGATCGGCATCGTGAACAACACCGTGTACGACCCCCT | |
| | GCAGCCCGAGCTGGACAGCTTCAAGGAGGAGCTGGACAAGTACTTCAA | |
| | GAACCACACCAGCCCCGACGTGGACCTGGGCGACATCAGCGGCATCA | |
| | ACGCCAGCGTGGTGAACATCCAGAAGGAGATCGACAGGCTGAACGAG | |
| | GTGGCCAAGAACCTGAACGAGAGCCTGATCGACCTGCAGGAGCTGGG | |
| | CAAGTACGAGCAGTACATCAAGTGGCCCTGGTACATCTGGCTGGGCTT | |
| | CATCGCCGGCCTGATCGCCATCGTGATGGTGACCATCATGCTGTGCTG | |
| | CATGACCAGCTGCTGCAGCTGCCTGAAGGGCTGCTGCAGCTGCGGCA | |
| | GCTGCTGCAAGTTCGACGAGGACGACAGCGAGCCCGTGCTGAAGGGC | |
| | GTGAAGCTGCACTACACCGGAAGCGGAGCCACGAACTTCTCTCTGTTA | |
| | AAGCAAGCAGGAGATGTTGAAGAAAACCCCGGGCCTGCCCCCACCAAG | |
| | GTGACCTTCGGCGACGACACCGTGATCGAGGTGCAGGGCTACAAGAG | |
| | CGTGAACATCACCTTCGAGCTGGACGAGAGGATCGACAAGGTGCTGAA | |
| | CGAGAAGTGCAGCGCCTACACCGTGGAGCTGGGCACCGAGGTGAACG | |
| | AGTTCGCCTGCGTGGTGGCCGACGCCGTGATCAAGACCCTGCAGCCC | |
| | GTGAGCGAGCTGCTGACCCCCCTGGGCATCGACCTGGACGAGTGGAG | |
| | CATGGCCACCTACTACCTGTTCGACGAGAGCGGCGAGTTCAAGCTGGC | |
| | CAGCCACATGTACTGCAGCTTCTACCCCCCCGACGAGGACGAGGAGGA | |
| | GGGCGACTGCGAGGAGGAGGAGTTCGAGCCCAGCACCCAGTACGAGT | |
| | ACGGCACCGAGGACGACTACCAGGGCAAGCCCCTGGAGTTCGGCGCC | |
| | ACCAGCGCCGCCCTGCAGCCCGAGGAGGAGCAGGAGGAGGACTGGC | |
| | TGGACGACGACAGCCAGCAGACCGTGGGCCAGCAGGACGGCAGCGA | |
| | GGACAACCAGACCACCACCATCCAGACCATCGTGGAGGTGCAGCCCCA | |
| | GCTGGAGATGGAGCTGACCCCCGTGGTGCAGACCATCGAGGTGAACA | |
| | GCTTCAGCGGCTACCTGAAGCTGACCGACAACGTGTACATCAAGAACG | |
| | CCGACATCGTGGAGGAGGCCAAGAAGGTGAAGCCCACCGTGGTGGTG | |
| | AACGCCGCCAACGTGTACCTGAAGCACGGCGGCGGCGTGGCCGGCGC | |
| | CCTGAACAAGGCCACCAACAACGCCATGCAGGTGGAGAGCGACGACTA | |
| | CATCGCCACCAACGGCCCCCTGAAGGTGGGCGGCAGCTGCGTGCTGA | |
| | GCGGCCACAACCTGGCCAAGCACTGCCTGCACGTGGTGGGCCCCAAC | |
| | GTGAACAAGGGCGAGGACATCCAGCTGCTGAAGAGCGCCTACGAGAA | |
| | CTTCAACCAGCACGAGGTGCTGCTGGCCCCCCTGCTGAGCGCCGGCA | |
| | TCTTCGGCGCCGACCCCATCCACAGCCTGAGGGTGTGCGTGGACACC | |
| | GTGAGGACCAACGTGTACCTGGCCGTGTTCGACAAGAACCTGTACGAC | |
| | AAGCTGGTGAGCAGCTTCCTGGAGATGAAGAGCGAGAAGCAGGTGGA | |
| | GCAGAAGATCGCCGAGATCCCCAAGGAGGAGGTGAAGCCCTTCATCAC | |
| | CGAGAGCAAGCCCAGCGTGGAGCAGAGGAAGCAGGACGACAAGAAGA | |
| | TCAAGGCCTGCGTGGAGGAGGTGACCACCACCCTGGAGGAGACCAAG | |
| | TTCCTGACCGAGAACCTGCTGCTGTACATCGACATCAACGGCAACCTG | |
| | CACCCCGACAGCGCCACCCTGGTGAGCGACATCGACATCACCTTCCTG | |
| | AAGAAGGACGCCCCCTACATCGTGGGCGACGTGGTGCAGGAGGGCGT | |
| | GCTGACCGCCGTGGTGATCCCCACCAAGAAGGCCGGCGGCACCACCG | |
| | AGATGCTGGCCAAGGCCCTGAGGAAGGTGCCCACCGACAACTACATCA | |
| | CCACCTACCCCGGCCAGGGCCTGAACGGCTACACCGTGGAGGAGGCC | |
| | AAGACCGTGCTGAAGAAGTGCAAGAGCGCCTTCTACATCCTGCCCAGC | |
| | ATCATCAGCAACGAGAAGCAGGAGATCCTGGGCACCGTGAGCTGGAAC | |
| | CTGAGGGAGATGCTGGCCCACGCCGAGGAGACCAGGAAGCTGATGCC | |
| | CGTGTGCGTGGAGACCAAGGCCATCGTGAGCACCATCCAGAGGAAGTA | |
| | CAAGGGCATCAAGATCCAGGAGGGCGTGGTGGACTACGGCGCCAGGT | |
| | TCTACTTCTACACCAGCAAGACCACCGTGGCCAGCCTGATCAACACCCT | |
| | GAACGACCTGAACGAGACCCTGGTGACCATGCCCCTGGGCTACGTGAC | |
| | CCACGGCCTGAACCTGGAGGAGGCCGCCAGGTACATGAGGAGCCTGA | |
| | AGGTGCCCGCCACCGTGAGCGTGAGCAGCCCCGACGCCGTGACCGCC | |
| | TACAACGGCTACCTGACCAGCAGCAGCAAGACCCCCGAGGAGCACTTC | |
| | ATCGAGACCATCAGCCTGGCCGGCAGCTACAAGGACTGGAGCTACAGC | |
| | GGCCAGAGCACCCAGCTGGGCATCGAGTTCCTGAAGAGGGGCGACAA | |
| | GAGCGTGTACTACACCAGCAACCCCACCACCTTCCACCTGGACGGCGA | |
| | GGTGATCACCTTCGACAACCTGAAGACCCTGCTGAGCCTGAGGGAGGT | |
| | GAGGACCATCAAGGTGTTCACCACCGTGGACAACATCAACCTGCACTG | |
| | ATGA | |
| 60 | ATGTTCGTGTTCCTGGTGCTGCTGCCCCTGGTGAGCAGCCAGTGCGTG | 345 |
| Pan-CoV | AACCTGACCACCAGGACCCAGCTGCCCCCCGCCTACACCAACAGCTTC | |
| (B6.2) | ACCAGGGGCGTGTACTACCCCGACAAGGTGTTCAGGAGCAGCGTGCT | |
| | GCACAGCACCCAGGACCTGTTCCTGCCCTTCTTCAGCAACGTGACCTG | |
| | GTTCCACGCCATCCACGTGAGCGGCACCAACGGCACCAAGAGGTTCGA | |
| | CAACCCCGTGCTGCCCTTCAACGACGGCGTGTACTTCGCCAGCACCGA | |
| | GAAGAGCAACATCATCAGGGGCTGGATCTTCGGCACCACCCTGGACAG | |
| | CAAGACCCAGAGCCTGCTGATCGTGAACAACGCCACCAACGTGGTGAT | |
| | CAAGGTGTGCGAGTTCCAGTTCTGCAACGACCCCTTCCTGGGCGTGTA | |
| | CTACCACAAGAACAACAAGAGCTGGATGGAGAGCGAGTTCAGGGTGTA | |
| | CAGCAGCGCCAACAACTGCACCTTCGAGTACGTGAGCCAGCCCTTCCT | |
| | GATGGACCTGGAGGGCAAGCAGGGCAACTTCAAGAACCTGAGGGAGT | |
| | TCGTGTTCAAGAACATCGACGGCTACTTCAAGATCTACAGCAAGCACAC | |
| | CCCCATCAACCTGGTGAGGGACCTGCCCCAGGGCTTCAGCGCCCTGG | |
| | AGCCCCTGGTGGACCTGCCCATCGGCATCAACATCACCAGGTTCCAGA | |
| | CCCTGCTGGCCCTGCACAGGAGCTACCTGACCCCCGGCGACAGCAGC | |
| | AGCGGCTGGACCGCCGGCGCCGCCGCCTACTACGTGGGCTACCTGCA | |
| | GCCCAGGACCTTCCTGCTGAAGTACAACGAGAACGGCACCATCACCGA | |
| | CGCCGTGGACTGCGCCCTGGACCCCCTGAGCGAGACCAAGTGCACCC | |
| | TGAAGAGCTTCACCGTGGAGAAGGGCATCTACCAGACCAGCAACTTCA | |
| | GGGTGCAGCCCACCGAGAGCATCGTGAGGTTCCCCAACATCACCAACC | |
| | TGTGCCCCTTCGGCGAGGTGTTCAACGCCACCAGGTTCGCCAGCGTGT | |
| | ACGCCTGGAACAGGAAGAGGATCAGCAACTGCGTGGCCGACTACAGC | |
| | GTGCTGTACAACAGCGCCAGCTTCAGCACCTTCAAGTGCTACGGCGTG | |
| | AGCCCCACCAAGCTGAACGACCTGTGCTTCACCAACGTGTACGCCGAC | |
| | AGCTTCGTGATCAGGGGCGACGAGGTGAGGCAGATCGCCCCCGGCCA | |
| | GACCGGCAAGATCGCCGACTACAACTACAAGCTGCCCGACGACTTCAC | |
| | CGGCTGCGTGATCGCCTGGAACAGCAACAACCTGGACAGCAAGGTGG | |
| | GCGGCAACTACAACTACCTGTACAGGCTGTTCAGGAAGAGCAACCTGA | |
| | AGCCCTTCGAGAGGGACATCAGCACCGAGATCTACCAGGCCGGCAGC | |
| | ACCCCCTGCAACGGCGTGGAGGGCTTCAACTGCTACTTCCCCCTGCAG | |
| | AGCTACGGCTTCCAGCCCACCAACGGCGTGGGCTACCAGCCCTACAG | |
| | GGTGGTGGTGCTGAGCTTCGAGCTGCTGCACGCCCCCGCCACCGTGT | |
| | GCGGCCCCAAGAAGAGCACCAACCTGGTGAAGAACAAGTGCGTGAACT | |
| | TCAACTTCAACGGCCTGACCGGCACCGGCGTGCTGACCGAGAGCAACA | |
| | AGAAGTTCCTGCCCTTCCAGCAGTTCGGCAGGGACATCGCCGACACCA | |
| | CCGACGCCGTGAGGGACCCCCAGACCCTGGAGATCCTGGACATCACC | |
| | CCCTGCAGCTTCGGCGGCGTGAGCGTGATCACCCCCGGCACCAACAC | |
| | CAGCAACCAGGTGGCCGTGCTGTACCAGGACGTGAACTGCACCGAGG | |
| | TGCCCGTGGCCATCCACGCCGACCAGCTGACCCCCACCTGGAGGGTG | |
| | TACAGCACCGGCAGCAACGTGTTCCAGACCAGGGCCGGCTGCCTGAT | |
| | CGGCGCCGAGCACGTGAACAACAGCTACGAGTGCGACATCCCCATCG | |
| | GCGCCGGCATCTGCGCCAGCTACCAGACCCAGACCAACAGCCCCGGC | |
| | AGCGCCAGCAGCGTGGCCAGCCAGAGCATCATCGCCTACACCATGAG | |
| | CCTGGGCGCCGAGAACAGCGTGGCCTACAGCAACAACAGCATCGCCA | |
| | TCCCCACCAACTTCACCATCAGCGTGACCACCGAGATCCTGCCCGTGA | |
| | GCATGACCAAGACCAGCGTGGACTGCACCATGTACATCTGCGGCGACA | |
| | GCACCGAGTGCAGCAACCTGCTGCTGCAGTACGGCAGCTTCTGCACCC | |
| | AGCTGAACAGGGCCCTGACCGGCATCGCCGTGGAGCAGGACAAGAAC | |
| | ACCCAGGAGGTGTTCGCCCAGGTGAAGCAGATCTACAAGACCCCCCCC | |
| | ATCAAGGACTTCGGCGGCTTCAACTTCAGCCAGATCCTGCCCGACCCC | |
| | AGCAAGCCCAGCAAGAGGAGCCCCATCGAGGACCTGCTGTTCAACAAG | |
| | GTGACCCTGGCCGACGCCGGCTTCATCAAGCAGTACGGCGACTGCCT | |
| | GGGCGACATCGCCGCCAGGGACCTGATCTGCGCCCAGAAGTTCAACG | |
| | GCCTGACCGTGCTGCCCCCCCTGCTGACCGACGAGATGATCGCCCAG | |
| | TACACCAGCGCCCTGCTGGCCGGCACCATCACCAGCGGCTGGACCTT | |
| | CGGCGCCGGCCCCGCCCTGCAGATCCCCTTCCCCATGCAGATGGCCT | |
| | ACAGGTTCAACGGCATCGGCGTGACCCAGAACGTGCTGTACGAGAACC | |
| | AGAAGCTGATCGCCAACCAGTTCAACAGCGCCATCGGCAAGATCCAGG | |
| | ACAGCCTGAGCAGCACCCCCAGCGCCCTGGGCAAGCTGCAGGACGTG | |
| | GTGAACCAGAACGCCCAGGCCCTGAACACCCTGGTGAAGCAGCTGAG | |
| | CAGCAACTTCGGCGCCATCAGCAGCGTGCTGAACGACATCCTGAGCAG | |
| | GCTGGACCCCCCCGAGGCCGAGGTGCAGATCGACAGGCTGATCACCG | |
| | GCAGGCTGCAGAGCCTGCAGACCTACGTGACCCAGCAGCTGATCAGG | |
| | GCCGCCGAGATCAGGGCCAGCGCCAACCTGGCCGCCACCAAGATGAG | |
| | CGAGTGCGTGCTGGGCCAGAGCAAGAGGGTGGACTTCTGCGGCAAGG | |
| | GCTACCACCTGATGAGCTTCCCCCAGAGCGCCCCCCACGGCGTGGTG | |
| | TTCCTGCACGTGACCTACGTGCCCGCCCAGGAGAAGAACTTCACCACC | |
| | GCCCCCGCCATCTGCCACGACGGCAAGGCCCACTTCCCCAGGGAGGG | |
| | CGTGTTCGTGAGCAACGGCACCCACTGGTTCGTGACCCAGAGGAACTT | |
| | CTACGAGCCCCAGATCATCACCACCGACAACACCTTCGTGAGCGGCAA | |
| | CTGCGACGTGGTGATCGGCATCGTGAACAACACCGTGTACGACCCCCT | |
| | GCAGCCCGAGCTGGACAGCTTCAAGGAGGAGCTGGACAAGTACTTCAA | |
| | GAACCACACCAGCCCCGACGTGGACCTGGGCGACATCAGCGGCATCA | |
| | ACGCCAGCGTGGTGAACATCCAGAAGGAGATCGACAGGCTGAACGAG | |
| | GTGGCCAAGAACCTGAACGAGAGCCTGATCGACCTGCAGGAGCTGGG | |
| | CAAGTACGAGCAGTACATCAAGTGGCCCTGGTACATCTGGCTGGGCTT | |
| | CATCGCCGGCCTGATCGCCATCGTGATGGTGACCATCATGCTGTGCTG | |
| | CATGACCAGCTGCTGCAGCTGCCTGAAGGGCTGCTGCAGCTGCGGCA | |
| | GCTGCTGCAAGTTCGACGAGGACGACAGCGAGCCCGTGCTGAAGGGC | |
| | GTGAAGCTGCACTACACCGGAAGCGGAGCCACGAACTTCTCTCTGTTA | |
| | AAGCAAGCAGGAGATGTTGAAGAAAACCCCGGGCCTAGCGGCTTCAGG | |
| | AAGATGGCCTTCCCCAGCGGCAAGGTGGAGGGCTGCATGGTGCAGGT | |
| | GACCTGCGGCACCACCACCCTGAACGGCCTGTGGCTGGACGACGTGG | |
| | TGTACTGCCCCAGGCACGTGATCTGCACCAGCGAGGACATGCTGAACC | |
| | CCAACTACGAGGACCTGCTGATCAGGAAGAGCAACCACAACTTCCTGG | |
| | TGCAGGCCGGCAACGTGCAGCTGAGGGTGATCGGCCACAGCATGCAG | |
| | AACTGCGTGCTGAAGCTGAAGGTGGACACCGCCAACCCCAAGACCCCC | |
| | AAGTACAAGTTCGTGAGGATCCAGCCCGGCCAGACCTTCAGCGTGCTG | |
| | GCCTGCTACAACGGCAGCCCCAGCGGCGTGTACCAGTGCGCCATGAG | |
| | GCCCAACTTCACCATCAAGGGCAGCTTCCTGAACGGCAGCTGCGGCAG | |
| | CGTGGGCTTCAACATCGACTACGACTGCGTGAGCTTCTGCTACATGCA | |
| | CCACATGGAGCTGCCCACCGGCGTGCACGCCGGCACCGACCTGGAGG | |
| | GCAACTTCTACGGCCCCTTCGTGGACAGGCAGACCGCCCAGGCCGCC | |
| | GGCACCGACACCACCATCACCGTGAACGTGCTGGCCTGGCTGTACGC | |
| | CGCCGTGATCAACGGCGACAGGTGGTTCCTGAACAGGTTCACCACCAC | |
| | CCTGAACGACTTCAACCTGGTGGCCATGAAGTACAACTACGAGCCCCT | |
| | GACCCAGGACCACGTGGACATCCTGGGCCCCCTGAGCGCCCAGACCG | |
| | GCATCGCCGTGCTGGACATGTGCGCCAGCCTGAAGGAGCTGCTGCAG | |
| | AACGGCATGAACGGCAGGACCATCCTGGGCAGCGCCCTGCTGGAGGA | |
| | CGAGTTCACCCCCTTCGACGTGGTGAGGCAGTGCAGCGGCGTGACCTT | |
| | CCAGTGATGA | |
| 61 | ATGTTCGTGTTCCTGGTGCTGCTGCCCCTGGTGAGCAGCCAGTGCGTG | 346 |
| Pan-CoV | AACCTGACCACCAGGACCCAGCTGCCCCCCGCCTACACCAACAGCTTC | |
| (B6.3) | ACCAGGGGCGTGTACTACCCCGACAAGGTGTTCAGGAGCAGCGTGCT | |
| | GCACAGCACCCAGGACCTGTTCCTGCCCTTCTTCAGCAACGTGACCTG | |
| | GTTCCACGCCATCCACGTGAGCGGCACCAACGGCACCAAGAGGTTCGA | |
| | CAACCCCGTGCTGCCCTTCAACGACGGCGTGTACTTCGCCAGCACCGA | |
| | GAAGAGCAACATCATCAGGGGCTGGATCTTCGGCACCACCCTGGACAG | |
| | CAAGACCCAGAGCCTGCTGATCGTGAACAACGCCACCAACGTGGTGAT | |
| | CAAGGTGTGCGAGTTCCAGTTCTGCAACGACCCCTTCCTGGGCGTGTA | |
| | CTACCACAAGAACAACAAGAGCTGGATGGAGAGCGAGTTCAGGGTGTA | |
| | CAGCAGCGCCAACAACTGCACCTTCGAGTACGTGAGCCAGCCCTTCCT | |
| | GATGGACCTGGAGGGCAAGCAGGGCAACTTCAAGAACCTGAGGGAGT | |
| | TCGTGTTCAAGAACATCGACGGCTACTTCAAGATCTACAGCAAGCACAC | |
| | CCCCATCAACCTGGTGAGGGACCTGCCCCAGGGCTTCAGCGCCCTGG | |
| | AGCCCCTGGTGGACCTGCCCATCGGCATCAACATCACCAGGTTCCAGA | |
| | CCCTGCTGGCCCTGCACAGGAGCTACCTGACCCCCGGCGACAGCAGC | |
| | AGCGGCTGGACCGCCGGCGCCGCCGCCTACTACGTGGGCTACCTGCA | |
| | GCCCAGGACCTTCCTGCTGAAGTACAACGAGAACGGCACCATCACCGA | |
| | CGCCGTGGACTGCGCCCTGGACCCCCTGAGCGAGACCAAGTGCACCC | |
| | TGAAGAGCTTCACCGTGGAGAAGGGCATCTACCAGACCAGCAACTTCA | |
| | GGGTGCAGCCCACCGAGAGCATCGTGAGGTTCCCCAACATCACCAACC | |
| | TGTGCCCCTTCGGCGAGGTGTTCAACGCCACCAGGTTCGCCAGCGTGT | |
| | ACGCCTGGAACAGGAAGAGGATCAGCAACTGCGTGGCCGACTACAGC | |
| | GTGCTGTACAACAGCGCCAGCTTCAGCACCTTCAAGTGCTACGGCGTG | |
| | AGCCCCACCAAGCTGAACGACCTGTGCTTCACCAACGTGTACGCCGAC | |
| | AGCTTCGTGATCAGGGGCGACGAGGTGAGGCAGATCGCCCCCGGCCA | |
| | GACCGGCAAGATCGCCGACTACAACTACAAGCTGCCCGACGACTTCAC | |
| | CGGCTGCGTGATCGCCTGGAACAGCAACAACCTGGACAGCAAGGTGG | |
| | GCGGCAACTACAACTACCTGTACAGGCTGTTCAGGAAGAGCAACCTGA | |
| | AGCCCTTCGAGAGGGACATCAGCACCGAGATCTACCAGGCCGGCAGC | |
| | ACCCCCTGCAACGGCGTGGAGGGCTTCAACTGCTACTTCCCCCTGCAG | |
| | AGCTACGGCTTCCAGCCCACCAACGGCGTGGGCTACCAGCCCTACAG | |
| | GGTGGTGGTGCTGAGCTTCGAGCTGCTGCACGCCCCCGCCACCGTGT | |
| | GCGGCCCCAAGAAGAGCACCAACCTGGTGAAGAACAAGTGCGTGAACT | |
| | TCAACTTCAACGGCCTGACCGGCACCGGCGTGCTGACCGAGAGCAACA | |
| | AGAAGTTCCTGCCCTTCCAGCAGTTCGGCAGGGACATCGCCGACACCA | |
| | CCGACGCCGTGAGGGACCCCCAGACCCTGGAGATCCTGGACATCACC | |
| | CCCTGCAGCTTCGGCGGCGTGAGCGTGATCACCCCCGGCACCAACAC | |
| | CAGCAACCAGGTGGCCGTGCTGTACCAGGACGTGAACTGCACCGAGG | |
| | TGCCCGTGGCCATCCACGCCGACCAGCTGACCCCCACCTGGAGGGTG | |
| | TACAGCACCGGCAGCAACGTGTTCCAGACCAGGGCCGGCTGCCTGAT | |
| | CGGCGCCGAGCACGTGAACAACAGCTACGAGTGCGACATCCCCATCG | |
| | GCGCCGGCATCTGCGCCAGCTACCAGACCCAGACCAACAGCCCCGGC | |
| | AGCGCCAGCAGCGTGGCCAGCCAGAGCATCATCGCCTACACCATGAG | |
| | CCTGGGCGCCGAGAACAGCGTGGCCTACAGCAACAACAGCATCGCCA | |
| | TCCCCACCAACTTCACCATCAGCGTGACCACCGAGATCCTGCCCGTGA | |
| | GCATGACCAAGACCAGCGTGGACTGCACCATGTACATCTGCGGCGACA | |
| | GCACCGAGTGCAGCAACCTGCTGCTGCAGTACGGCAGCTTCTGCACCC | |
| | AGCTGAACAGGGCCCTGACCGGCATCGCCGTGGAGCAGGACAAGAAC | |
| | ACCCAGGAGGTGTTCGCCCAGGTGAAGCAGATCTACAAGACCCCCCCC | |
| | ATCAAGGACTTCGGCGGCTTCAACTTCAGCCAGATCCTGCCCGACCCC | |
| | AGCAAGCCCAGCAAGAGGAGCCCCATCGAGGACCTGCTGTTCAACAAG | |
| | GTGACCCTGGCCGACGCCGGCTTCATCAAGCAGTACGGCGACTGCCT | |
| | GGGCGACATCGCCGCCAGGGACCTGATCTGCGCCCAGAAGTTCAACG | |
| | GCCTGACCGTGCTGCCCCCCCTGCTGACCGACGAGATGATCGCCCAG | |
| | TACACCAGCGCCCTGCTGGCCGGCACCATCACCAGCGGCTGGACCTT | |
| | CGGCGCCGGCCCCGCCCTGCAGATCCCCTTCCCCATGCAGATGGCCT | |
| | ACAGGTTCAACGGCATCGGCGTGACCCAGAACGTGCTGTACGAGAACC | |
| | AGAAGCTGATCGCCAACCAGTTCAACAGCGCCATCGGCAAGATCCAGG | |
| | ACAGCCTGAGCAGCACCCCCAGCGCCCTGGGCAAGCTGCAGGACGTG | |
| | GTGAACCAGAACGCCCAGGCCCTGAACACCCTGGTGAAGCAGCTGAG | |
| | CAGCAACTTCGGCGCCATCAGCAGCGTGCTGAACGACATCCTGAGCAG | |
| | GCTGGACCCCCCCGAGGCCGAGGTGCAGATCGACAGGCTGATCACCG | |
| | GCAGGCTGCAGAGCCTGCAGACCTACGTGACCCAGCAGCTGATCAGG | |
| | GCCGCCGAGATCAGGGCCAGCGCCAACCTGGCCGCCACCAAGATGAG | |
| | CGAGTGCGTGCTGGGCCAGAGCAAGAGGGTGGACTTCTGCGGCAAGG | |
| | GCTACCACCTGATGAGCTTCCCCCAGAGCGCCCCCCACGGCGTGGTG | |
| | TTCCTGCACGTGACCTACGTGCCCGCCCAGGAGAAGAACTTCACCACC | |
| | GCCCCCGCCATCTGCCACGACGGCAAGGCCCACTTCCCCAGGGAGGG | |
| | CGTGTTCGTGAGCAACGGCACCCACTGGTTCGTGACCCAGAGGAACTT | |
| | CTACGAGCCCCAGATCATCACCACCGACAACACCTTCGTGAGCGGCAA | |
| | CTGCGACGTGGTGATCGGCATCGTGAACAACACCGTGTACGACCCCCT | |
| | GCAGCCCGAGCTGGACAGCTTCAAGGAGGAGCTGGACAAGTACTTCAA | |
| | GAACCACACCAGCCCCGACGTGGACCTGGGCGACATCAGCGGCATCA | |
| | ACGCCAGCGTGGTGAACATCCAGAAGGAGATCGACAGGCTGAACGAG | |
| | GTGGCCAAGAACCTGAACGAGAGCCTGATCGACCTGCAGGAGCTGGG | |
| | CAAGTACGAGCAGTACATCAAGTGGCCCTGGTACATCTGGCTGGGCTT | |
| | CATCGCCGGCCTGATCGCCATCGTGATGGTGACCATCATGCTGTGCTG | |
| | CATGACCAGCTGCTGCAGCTGCCTGAAGGGCTGCTGCAGCTGCGGCA | |
| | GCTGCTGCAAGTTCGACGAGGACGACAGCGAGCCCGTGCTGAAGGGC | |
| | GTGAAGCTGCACTACACCGGAAGCGGAGCCACGAACTTCTCTCTGTTA | |
| | AAGCAAGCAGGAGATGTTGAAGAAAACCCCGGGCCTAGCGCCGACGC | |
| | CCAGAGCTTCCTGAACAGGGTGTGCGGCGTGAGCGCCGCCAGGCTGA | |
| | CCCCCTGCGGCACCGGCACCAGCACCGACGTGGTGTACAGGGCCTTC | |
| | GACATCTACAACGACAAGGTGGCCGGCTTCGCCAAGTTCCTGAAGACC | |
| | AACTGCTGCAGGTTCCAGGAGAAGGACGAGGACGACAACCTGATCGAC | |
| | AGCTACTTCGTGGTGAAGAGGCACACCTTCAGCAACTACCAGCACGAG | |
| | GAGACCATCTACAACCTGCTGAAGGACTGCCCCGCCGTGGCCAAGCAC | |
| | GACTTCTTCAAGTTCAGGATCGACGGCGACATGGTGCCCCACATCAGC | |
| | AGGCAGAGGCTGACCAAGTACACCATGGCCGACCTGGTGTACGCCCT | |
| | GAGGCACTTCGACGAGGGCAACTGCGACACCCTGAAGGAGATCCTGG | |
| | TGACCTACAACTGCTGCGACGACGACTACTTCAACAAGAAGGACTGGT | |
| | ACGACTTCGTGGAGAACCCCGACATCCTGAGGGTGTACGCCAACCTGG | |
| | GCGAGAGGGTGAGGCAGGCCCTGCTGAAGACCGTGCAGTTCTGCGAC | |
| | GCCATGAGGAACGCCGGCATCGTGGGCGTGCTGACCCTGGACAACCA | |
| | GGACCTGAACGGCAACTGGTACGACTTCGGCGACTTCATCCAGACCAC | |
| | CCCCGGCAGCGGCGTGCCCGTGGTGGACAGCTACTACAGCCTGCTGA | |
| | TGCCCATCCTGACCCTGACCAGGGCCCTGACCGCCGAGAGCCACGTG | |
| | GACACCGACCTGACCAAGCCCTACATCAAGTGGGACCTGCTGAAGTAC | |
| | GACTTCACCGAGGAGAGGCTGAAGCTGTTCGACAGGTACTTCAAGTAC | |
| | TGGGACCAGACCTACCACCCCAACTGCGTGAACTGCCTGGACGACAGG | |
| | TGCATCCTGCACTGCGCCAACTTCAACGTGCTGTTCAGCACCGTGTTCC | |
| | CCCCCACCAGCTTCGGCCCCCTGGTGAGGAAGATCTTCGTGGACGGC | |
| | GTGCCCTTCGTGGTGAGCACCGGCTACCACTTCAGGGAGCTGGGCGT | |
| | GGTGCACAACCAGGACGTGAACCTGCACAGCAGCAGGCTGAGCTTCAA | |
| | GGAGCTGCTGGTGTACGCCGCCGACCCCGCCATGCACGCCGCCAGCG | |
| | GCAACCTGCTGCTGGACAAGAGGACCACCTGCTTCAGCGTGGCCGCC | |
| | CTGACCAACAACGTGGCCTTCCAGACCGTGAAGCCCGGCAACTTCAAC | |
| | AAGGACTTCTACGACTTCGCCGTGAGCAAGGGCTTCTTCAAGGAGGGC | |
| | AGCAGCGTGGAGCTGAAGCACTTCTTCTTCGCCCAGGACGGCAACGCC | |
| | GCCATCAGCGACTACGACTACTACAGGTACAACCTGCCCACCATGTGC | |
| | GACATCAGGCAGCTGCTGTTCGTGGTGGAGGTGGTGGACAAGTACTTC | |
| | GACTGCTACGACGGCGGCTGCATCAACGCCAACCAGGTGATCGTGAAC | |
| | AACCTGGACAAGAGCGCCGGCTTCCCCTTCAACAAGTGGGGCAAGGC | |
| | CAGGCTGTACTACGACAGCATGAGCTACGAGGACCAGGACGCCCTGTT | |
| | CGCCTACACCAAGAGGAACGTGATCCCCACCATCACCCAGATGAACCT | |
| | GAAGTACGCCATCAGCGCCAAGAACAGGGCCAGGACCGTGGCCGGCG | |
| | TGAGCATCTGCAGCACCATGACCAACAGGCAGTTCCACCAGAAGCTGC | |
| | TGAAGAGCATCGCCGCCACCAGGGGCGCCACCGTGGTGATCGGCACC | |
| | AGCAAGTTCTACGGCGGCTGGCACAACATGCTGAAGACCGTGTACAGC | |
| | GACGTGGAGAACCCCCACCTGATGGGCTGGGACTACCCCAAGTGCGA | |
| | CAGGGCCATGCCCAACATGCTGAGGATCATGGCCAGCCTGGTGCTGG | |
| | CCAGGAAGCACACCACCTGCTGCAGCCTGAGCCACAGGTTCTACAGGC | |
| | TGGCCAACGAGTGCGCCCAGGTGCTGAGCGAGATGGTGATGTGCGGC | |
| | GGCAGCCTGTACGTGAAGCCCGGCGGCACCAGCAGCGGCGACGCCAC | |
| | CACCGCCTACGCCAACAGCGTGTTCAACATCTGCCAGGCCGTGACCGC | |
| | CAACGTGAACGCCCTGCTGAGCACCGACGGCAACAAGATCGCCGACAA | |
| | GTACGTGAGGAACCTGCAGCACAGGCTGTACGAGTGCCTGTACAGGAA | |
| | CAGGGACGTGGACACCGACTTCGTGAACGAGTTCTACGCCTACCTGAG | |
| | GAAGCACTTCAGCATGATGATCCTGAGCGACGACGCCGTGGTGTGCTT | |
| | CAACAGCACCTACGCCAGCCAGGGCCTGGTGGCCAGCATCAAGAACTT | |
| | CAAGAGCGTGCTGTACTACCAGAACAACGTGTTCATGAGCGAGGCCAA | |
| | GTGCTGGACCGAGACCGACCTGACCAAGGGCCCCCACGAGTTCTGCA | |
| | GCCAGCACACCATGCTGGTGAAGCAGGGCGACGACTACGTGTACCTG | |
| | CCCTACCCCGACCCCAGCAGGATCCTGGGCGCCGGCTGCTTCGTGGA | |
| | CGACATCGTGAAGACCGACGGCACCCTGATGATCGAGAGGTTCGTGAG | |
| | CCTGGCCATCGACGCCTACCCCCTGACCAAGCACCCCAACCAGGAGTA | |
| | CGCCGACGTGTTCCACCTGTACCTGCAGTACATCAGGAAGCTGCACGA | |
| | CGAGCTGACCGGCCACATGCTGGACATGTACAGCGTGATGCTGACCAA | |
| | CGACAACACCAGCAGGTACTGGGAGCCCGAGTTCTACGAGGCCATGTA | |
| | CACCCCCCACACCGTGCTGCAGTGATGA | |
| 62 | ATGAGCGACAACGGCCCCCAGAACCAGAGGAACGCCCCCAGGATCAC | 347 |
| Pan-CoV (B3) | CTTCGGCGGCCCCAGCGACAGCACCGGCAGCAACCAGAACGGCGAGA | |
| | GGAGCGGCGCCAGGAGCAAGCAGAGGAGGCCCCAGGGCCTGCCCAA | |
| | CAACACCGCCAGCTGGTTCACCGCCCTGACCCAGCACGGCAAGGAGG | |
| | ACCTGAAGTTCCCCAGGGGCCAGGGCGTGCCCATCAACACCAACAGCA | |
| | GCCCCGACGACCAGATCGGCTACTACAGGAGGGCCACCAGGAGGATC | |
| | AGGGGCGGCGACGGCAAGATGAAGGACCTGAGCCCCAGGTGGTACTT | |
| | CTACTACCTGGGCACCGGCCCCGAGGCCGGCCTGCCCTACGGCGCCA | |
| | ACAAGGACGGCATCATCTGGGTGGCCACCGAGGGCGCCCTGAACACC | |
| | CCCAAGGACCACATCGGCACCAGGAACCCCGCCAACAACGCCGCCAT | |
| | CGTGCTGCAGCTGCCCCAGGGCACCACCCTGCCCAAGGGCTTCTACG | |
| | CCGAGGGCAGCAGGGGCGGCAGCCAGGCCAGCAGCAGGAGCAGCAG | |
| | CAGGAGCAGGAACAGCAGCAGGAACAGCACCCCCGGCAGCAGCAGGG | |
| | GCACCAGCCCCGCCAGGATGGCCGGCAACGGCGGCGACGCCGCCCT | |
| | GGCCCTGCTGCTGCTGGACAGGCTGAACCAGCTGGAGAGCAAGATGA | |
| | GCGGCAAGGGCCAGCAGCAGCAGGGCCAGACCGTGACCAAGAAGAGC | |
| | GCCGCCGAGGCCAGCAAGAAGCCCAGGCAGAAGAGGACCGCCACCAA | |
| | GGCCTACAACGTGACCCAGGCCTTCGGCAGGAGGGGCCCCGAGCAGA | |
| | CCCAGGGCAACTTCGGCGACCAGGAGCTGATCAGGCAGGGCACCGAC | |
| | TACAAGCACTGGCCCCAGATCGCCCAGTTCGCCCCCAGCGCCAGCGC | |
| | CTTCTTCGGCATGAGCAGGATCGGCATGGAGGTGACCCCCAGCGGCA | |
| | CCTGGCTGACCTACACCGGCGCCATCAAGCTGGACGACAAGGACCCC | |
| | AACTTCAAGGACCAGGTGATCCTGCTGAACAAGCACATCGACGCCTAC | |
| | AAGACCTTCCCCCCCACCGAGCCCAAGAAGGACAAGAAGAAGAAGGCC | |
| | GACGAGACCCAGGCCCTGCCCCAGAGGCAGAAGAAGCAGCAGACCGT | |
| | GACCCTGCTGCCCGCCGCCGACCTGGACGACTTCAGCAAGCAGCTGC | |
| | AGCAGAGCATGAGCAGCGCCGACAGCACCCAGGCCTGATGA | |

As mentioned above, the present invention is not limited to the examples in Table 9. In some embodiments, vaccine candidates may comprise various pieces (e.g. promoters, proteins, adjuvants) as shown described herein.

The present invention may further feature a pan-coronavirus recombinant vaccine composition, the composition comprising one or more large sequences, wherein each of the one or more large sequences comprise conserved regions of a coronavirus. For example, Table 10 features non-limiting examples of proteins comprising conserved region from a coronavirus that may be used in the compositions described herein.

Table 10 shows non-limiting examples of proteins that may be used to create a vaccine composition described herein. In some embodiments, proteins listed below may be arranged in a plurality of combinations. In some embodiments, the proteins may be directly linked together. In other embodiments, the proteins are linked together via a linker.

TABLET0

| | | SEQ ID |
|---|---|---|
| Proteins | Sequence | NO: |
| Spike glycoprotein | ATGTTCGTGTTCCTGGTGCTGCTGCCCCTGGTGAGCAGCCAGTG | 148 |
| with 6 stabilizing | CGTGAACCTGACCACCCGGACCCAGCTGCCACCAGCCTACACC | |
| mutations | AACAGCTTCACCCGGGGCGTCTACTACCCCGACAAGGTGTTCCG | |
| (HexaPro) | GAGCAGCGTCCTGCACAGCACCCAGGACCTGTTCCTGCCCTTCT | |
| | TCAGCAACGTGACCTGGTTCCACGCCATCCACGTGAGCGGCAC | |
| | CAACGGCACCAAGCGGTTCGACAACCCCGTGCTGCCCTTCAAC | |
| | GACGGCGTGTACTTCGCCAGCACCGAGAAGAGCAACATCATCC | |
| | GGGGCTGGATCTTCGGCACCACCCTGGACAGCAAGACCCAGAG | |
| | CCTGCTGATCGTGAATAACGCCACCAACGTGGTGATCAAGGTGT | |
| | GCGAGTTCCAGTTCTGCAACGACCCCTTCCTGGGCGTGTACTAC | |
| | CACAAGAACAACAAGAGCTGGATGGAGAGCGAGTTCCGGGTGT | |
| | ACAGCAGCGCCAACAACTGCACCTTCGAGTACGTGAGCCAGCC | |
| | CTTCCTGATGGACCTGGAGGGCAAGCAGGGCAACTTCAAGAACC | |
| | TGCGGGAGTTCGTGTTCAAGAACATCGACGGCTACTTCAAGATC | |
| | TACAGCAAGCACACCCCAATCAACCTGGTGCGGGATCTGCCCCA | |
| | GGGCTTCTCAGCCCTGGAGCCCCTGGTGGACCTGCCCATCGGC | |
| | ATCAACATCACCCGGTTCCAGACCCTGCTGGCCCTGCACCGGAG | |
| | CTACCTGACCCCAGGCGACAGCAGCAGCGGGTGGACAGCAGGC | |
| | GCGGCTGCTTACTACGTGGGCTACCTGCAGCCCCGGACCTTCCT | |
| | GCTGAAGTACAACGAGAACGGCACCATCACCGACGCCGTGGAC | |
| | TGCGCCCTGGACCCTCTGAGCGAGACCAAGTGCACCCTGAAGA | |
| | GCTTCACCGTGGAGAAGGGCATCTACCAGACCAGCAACTTCCGG | |
| | GTGCAGCCCACCGAGAGCATCGTGCGGTTCCCCAACATCACCAA | |
| | CCTGTGCCCCTTCGGCGAGGTGTTCAACGCCACCCGGTTCGCC | |
| | AGCGTGTACGCCTGGAACCGGAAGCGGATCAGCAACTGCGTGG | |
| | CCGACTACAGCGTGCTGTACAACAGCGCCAGCTTCAGCACCTTC | |
| | AAGTGCTACGGCGTGAGCCCCACCAAGCTGAACGACCTGTGCTT | |
| | CACCAACGTGTACGCCGACAGCTTCGTGATCCGTGGCGACGAG | |
| | GTGCGGCAGATCGCACCCGGCCAGACAGGCAAGATCGCCGACT | |
| | ACAACTACAAGCTGCCCGACGACTTCACCGGCTGCGTGATCGCC | |
| | TGGAACAGCAACAACCTCGACAGCAAGGTGGGCGGCAACTACA | |
| | ACTACCTGTACCGGCTGTTCCGGAAGAGCAACCTGAAGCCCTTC | |
| | GAGCGGGACATCAGCACCGAGATCTACCAAGCCGGCTCCACCC | |
| | CTTGCAACGGCGTGGAGGGCTTCAACTGCTACTTCCCTCTGCAG | |
| | AGCTACGGCTTCCAGCCCACCAACGGCGTGGGCTACCAGCCCT | |
| | ACCGGGTGGTGGTGCTGAGCTTCGAGCTGCTGCACGCCCCAGC | |
| | CACCGTGTGTGGCCCCAAGAAGAGCACCAACCTGGTGAAGAAC | |
| | AAGTGCGTGAACTTCAACTTCAACGGCCTTACCGGCACCGGCGT | |
| | GCTGACCGAGAGCAACAAGAAATTCCTGCCCTTTCAGCAGTTCG | |
| | GCCGGGACATCGCCGACACCACCGACGCTGTGCGGGATCCCCA | |
| | GACCCTGGAGATCCTGGACATCACCCCTTGCAGCTTCGGCGGC | |
| | GTGAGCGTGATCACCCCAGGCACCAACACCAGCAACCAGGTGG | |
| | CCGTGCTGTACCAGGACGTGAACTGCACCGAGGTGCCCGTGGC | |
| | CATCCACGCCGACCAGCTGACACCCACCTGGCGGGTCTACAGC | |
| | ACCGGCAGCAACGTGTTCCAGACCCGGGCCGGTTGCCTGATCG | |
| | GCGCCGAGCACGTGAACAACAGCTACGAGTGCGACATCCCCAT | |
| | CGGCGCCGGCATCTGTGCCAGCTACCAGACCCAGACCAATTCA | |
| | CCCGGCAGCGCCAGCAGCGTGGCCAGCCAGAGCATCATCGCCT | |
| | ACACCATGAGCCTGGGCGCCGAGAACAGCGTGGCCTACAGCAA | |
| | CAACAGCATCGCCATCCCCACCAACTTCACCATCAGCGTGACCA | |
| | CCGAGATTCTGCCCGTGAGCATGACCAAGACCAGCGTGGACTG | |
| | CACCATGTACATCTGCGGCGACAGCACCGAGTGCAGCAACCTG | |
| | CTGCTGCAGTACGGCAGCTTCTGCACCCAGCTGAACCGGGCCC | |
| | TGACCGGCATCGCCGTGGAGCAGGACAAGAACACCCAGGAGGT | |
| | GTTCGCCCAGGTGAAGCAGATCTACAAGACCCCTCCCATCAAGG | |
| | ACTTCGGCGGCTTCAACTTCAGCCAGATCCTGCCCGACCCCAGC | |
| | AAGCCCAGCAAGCGGAGCCCCATCGAGGACCTGCTGTTCAACA | |
| | AGGTGACCCTAGCCGACGCCGGCTTCATCAAGCAGTACGGCGA | |
| | CTGCCTCGGCGACATAGCCGCCCGGGACCTGATCTGCGCCCAG | |
| | AAGTTCAACGGCCTGACCGTGCTGCCTCCCCTGCTGACCGACGA | |
| | GATGATCGCCCAGTACACCAGCGCCCTGTTAGCCGGAACCATCA | |
| | CCAGCGGCTGGACTTTCGGCGCTGGCCCCGCTCTGCAGATCCC | |
| | CTTCCCCATGCAGATGGCCTACCGGTTCAACGGCATCGGCGTGA | |
| | CCCAGAACGTGCTGTACGAGAACCAGAAGCTGATCGCCAACCAG | |
| | TTCAACAGCGCCATCGGCAAGATCCAGGACAGCCTGAGCAGCA | |
| | CCCCTAGCGCCCTGGGCAAGCTGCAGGACGTGGTGAACCAGAA | |
| | CGCCCAGGCCCTGAACACCCTGGTGAAGCAGCTGAGCAGCAAC | |
| | TTCGGCGCCATCAGCAGCGTGCTGAACGACATCCTGAGCCGGC | |
| | TGGACCCTCCCGAGGCCGAGGTGCAGATCGACCGGCTGATCAC | |
| | TGGCCGGCTGCAGAGCCTGCAGACCTACGTGACCCAGCAGCTG | |
| | ATCCGGGCCGCCGAGATTCGGGCCAGCGCCAACCTGGCCGCCA | |
| | CCAAGATGAGCGAGTGCGTGCTGGGCCAGAGCAAGCGGGTGGA | |
| | CTTCTGCGGCAAGGGCTACCACCTGATGAGCTTTCCCCAGAGCG | |
| | CACCCCACGGAGTGGTGTTCCTGCACGTGACCTACGTGCCCGC | |
| | CCAGGAGAAGAACTTCACCACCGCCCCAGCCATCTGCCACGAC | |
| | GGCAAGGCCCACTTTCCCCGGGAGGGCGTGTTCGTGAGCAACG | |
| | GCACCCACTGGTTCGTGACCCAGCGGAACTTCTACGAGCCCCA | |
| | GATCATCACCACCGACAACACCTTCGTGAGCGGCAACTGCGACG | |
| | TGGTGATCGGCATCGTGAACAACACCGTGTACGATCCCCTGCAG | |
| | CCCGAGCTGGACAGCTTCAAGGAGGAGCTGGACAAGTACTTCAA | |
| | GAATCACACCAGCCCCGACGTGGACCTGGGCGACATCAGCGGC | |
| | ATCAACGCCAGCGTGGTGAACATCCAGAAGGAGATCGATCGGCT | |
| | GAACGAGGTGGCCAAGAACCTGAACGAGAGCCTGATCGACCTG | |
| | CAGGAGCTGGGCAAGTACGAGCAGTACATCAAGTGGCCCTGGT | |
| | ACATCTGGCTGGGCTTCATCGCCGGCCTGATCGCCATCGTGATG | |
| | GTGACCATCATGCTGTGCTGCATGACCAGCTGCTGCAGCTGCCT | |
| | GAAGGGCTGTTGCAGCTGCGGCAGCTGCTGCAAGTTCGACGAG | |
| | GACGACAGCGAGCCCGTGCTGAAGGGCGTGAAGCTGCACTACA | |
| | CCTGATAATAGGCTGGAGCCTCGGTGGCCTAGCTTCTTGCCCCT | |
| | TGGGCCTCCCCCCAGCCCCTCCTCCCCTTCCTGCACCCGTACCC | |
| | CCGTGGTCTTTGAATAAAGTCTGAGTGGGCGGCAAAAAAAAA | |
| Spike | ATGTTCGTGTTCCTGGTGCTGCTGCCCCTGGTGAGCAGCCAGTG | 143 |
| glycoprotein | CGTGAACCTGACCACCCGGACCCAGCTGCCACCAGCCTACACC | |
| with one | AACAGCTTCACCCGGGGCGTCTACTACCCCGACAAGGTGTTCCG | |
| stabilizing | GAGCAGCGTCCTGCACAGCACCCAGGACCTGTTCCTGCCCTTCT | |
| mutations | TCAGCAACGTGACCTGGTTCCACGCCATCCACGTGAGCGGCAC | |
| | CAACGGCACCAAGCGGTTCGACAACCCCGTGCTGCCCTTCAAC | |
| | GACGGCGTGTACTTCGCCAGCACCGAGAAGAGCAACATCATCC | |
| | GGGGCTGGATCTTCGGCACCACCCTGGACAGCAAGACCCAGAG | |
| | CCTGCTGATCGTGAATAACGCCACCAACGTGGTGATCAAGGTGT | |
| | GCGAGTTCCAGTTCTGCAACGACCCCTTCCTGGGCGTGTACTAC | |
| | CACAAGAACAACAAGAGCTGGATGGAGAGCGAGTTCCGGGTGT | |
| | ACAGCAGCGCCAACAACTGCACCTTCGAGTACGTGAGCCAGCC | |
| | CTTCCTGATGGACCTGGAGGGCAAGCAGGGCAACTTCAAGAACC | |
| | TGCGGGAGTTCGTGTTCAAGAACATCGACGGCTACTTCAAGATC | |
| | TACAGCAAGCACACCCCAATCAACCTGGTGCGGGATCTGCCCCA | |
| | GGGCTTCTCAGCCCTGGAGCCCCTGGTGGACCTGCCCATCGGC | |
| | ATCAACATCACCCGGTTCCAGACCCTGCTGGCCCTGCACCGGAG | |
| | CTACCTGACCCCAGGCGACAGCAGCAGCGGGTGGACAGCAGGC | |
| | GCGGCTGCTTACTACGTGGGCTACCTGCAGCCCCGGACCTTCCT | |
| | GCTGAAGTACAACGAGAACGGCACCATCACCGACGCCGTGGAC | |
| | TGCGCCCTGGACCCTCTGAGCGAGACCAAGTGCACCCTGAAGA | |
| | GCTTCACCGTGGAGAAGGGCATCTACCAGACCAGCAACTTCCGG | |
| | GTGCAGCCCACCGAGAGCATCGTGCGGTTCCCCAACATCACCAA | |
| | CCTGTGCCCCTTCGGCGAGGTGTTCAACGCCACCCGGTTCGCC | |
| | AGCGTGTACGCCTGGAACCGGAAGCGGATCAGCAACTGCGTGG | |
| | CCGACTACAGCGTGCTGTACAACAGCGCCAGCTTCAGCACCTTC | |
| | AAGTGCTACGGCGTGAGCCCCACCAAGCTGAACGACCTGTGCTT | |
| | CACCAACGTGTACGCCGACAGCTTCGTGATCCGTGGCGACGAG | |
| | GTGCGGCAGATCGCACCCGGCCAGACAGGCAAGATCGCCGACT | |
| | ACAACTACAAGCTGCCCGACGACTTCACCGGCTGCGTGATCGCC | |
| | TGGAACAGCAACAACCTCGACAGCAAGGTGGGCGGCAACTACA | |
| | ACTACCTGTACCGGCTGTTCCGGAAGAGCAACCTGAAGCCCTTC | |
| | GAGCGGGACATCAGCACCGAGATCTACCAAGCCGGCTCCACCC | |
| | CTTGCAACGGCGTGGAGGGCTTCAACTGCTACTTCCCTCTGCAG | |
| | AGCTACGGCTTCCAGCCCACCAACGGCGTGGGCTACCAGCCCT | |
| | ACCGGGTGGTGGTGCTGAGCTTCGAGCTGCTGCACGCCCCAGC | |
| | CACCGTGTGTGGCCCCAAGAAGAGCACCAACCTGGTGAAGAAC | |
| | AAGTGCGTGAACTTCAACTTCAACGGCCTTACCGGCACCGGCGT | |
| | GCTGACCGAGAGCAACAAGAAATTCCTGCCCTTTCAGCAGTTCG | |
| | GCCGGGACATCGCCGACACCACCGACGCTGTGCGGGATCCCCA | |
| | GACCCTGGAGATCCTGGACATCACCCCTTGCAGCTTCGGCGGC | |
| | GTGAGCGTGATCACCCCAGGCACCAACACCAGCAACCAGGTGG | |
| | CCGTGCTGTACCAGGACGTGAACTGCACCGAGGTGCCCGTGGC | |
| | CATCCACGCCGACCAGCTGACACCCACCTGGCGGGTCTACAGC | |
| | ACCGGCAGCAACGTGTTCCAGACCCGGGCCGGTTGCCTGATCG | |
| | GCGCCGAGCACGTGAACAACAGCTACGAGTGCGACATCCCCAT | |
| | CGGCGCCGGCATCTGTGCCAGCTACCAGACCCAGACCAATTCA | |
| | CCCGGCAGCGCCAGCAGCGTGGCCAGCCAGAGCATCATCGCCT | |
| | ACACCATGAGCCTGGGCGCCGAGAACAGCGTGGCCTACAGCAA | |
| | CAACAGCATCGCCATCCCCACCAACTTCACCATCAGCGTGACCA | |
| | CCGAGATTCTGCCCGTGAGCATGACCAAGACCAGCGTGGACTG | |
| | CACCATGTACATCTGCGGCGACAGCACCGAGTGCAGCAACCTG | |
| | CTGCTGCAGTACGGCAGCTTCTGCACCCAGCTGAACCGGGCCC | |
| | TGACCGGCATCGCCGTGGAGCAGGACAAGAACACCCAGGAGGT | |
| | GTTCGCCCAGGTGAAGCAGATCTACAAGACCCCTCCCATCAAGG | |
| | ACTTCGGCGGCTTCAACTTCAGCCAGATCCTGCCCGACCCCAGC | |
| | AAGCCCAGCAAGCGGAGCCCCATCGAGGACCTGCTGTTCAACA | |
| | AGGTGACCCTAGCCGACGCCGGCTTCATCAAGCAGTACGGCGA | |
| | CTGCCTCGGCGACATAGCCGCCCGGGACCTGATCTGCGCCCAG | |
| | AAGTTCAACGGCCTGACCGTGCTGCCTCCCCTGCTGACCGACGA | |
| | GATGATCGCCCAGTACACCAGCGCCCTGTTAGCCGGAACCATCA | |
| | CCAGCGGCTGGACTTTCGGCGCTGGCCCCGCTCTGCAGATCCC | |
| | CTTCCCCATGCAGATGGCCTACCGGTTCAACGGCATCGGCGTGA | |
| | CCCAGAACGTGCTGTACGAGAACCAGAAGCTGATCGCCAACCAG | |
| | TTCAACAGCGCCATCGGCAAGATCCAGGACAGCCTGAGCAGCA | |
| | CCCCTAGCGCCCTGGGCAAGCTGCAGGACGTGGTGAACCAGAA | |
| | CGCCCAGGCCCTGAACACCCTGGTGAAGCAGCTGAGCAGCAAC | |
| | TTCGGCGCCATCAGCAGCGTGCTGAACGACATCCTGAGCCGGC | |
| | TGGACCCTCCCGAGGCCGAGGTGCAGATCGACCGGCTGATCAC | |
| | TGGCCGGCTGCAGAGCCTGCAGACCTACGTGACCCAGCAGCTG | |
| | ATCCGGGCCGCCGAGATTCGGGCCAGCGCCAACCTGGCCGCCA | |
| | CCAAGATGAGCGAGTGCGTGCTGGGCCAGAGCAAGCGGGTGGA | |
| | CTTCTGCGGCAAGGGCTACCACCTGATGAGCTTTCCCCAGAGCG | |
| | CACCCCACGGAGTGGTGTTCCTGCACGTGACCTACGTGCCCGC | |
| | CCAGGAGAAGAACTTCACCACCGCCCCAGCCATCTGCCACGAC | |
| | GGCAAGGCCCACTTTCCCCGGGAGGGCGTGTTCGTGAGCAACG | |
| | GCACCCACTGGTTCGTGACCCAGCGGAACTTCTACGAGCCCCA | |
| | GATCATCACCACCGACAACACCTTCGTGAGCGGCAACTGCGACG | |
| | TGGTGATCGGCATCGTGAACAACACCGTGTACGATCCCCTGCAG | |
| | CCCGAGCTGGACAGCTTCAAGGAGGAGCTGGACAAGTACTTCAA | |
| | GAATCACACCAGCCCCGACGTGGACCTGGGCGACATCAGCGGC | |
| | ATCAACGCCAGCGTGGTGAACATCCAGAAGGAGATCGATCGGCT | |
| | GAACGAGGTGGCCAAGAACCTGAACGAGAGCCTGATCGACCTG | |
| | CAGGAGCTGGGCAAGTACGAGCAGTACATCAAGTGGCCCTGGT | |
| | ACATCTGGCTGGGCTTCATCGCCGGCCTGATCGCCATCGTGATG | |
| | GTGACCATCATGCTGTGCTGCATGACCAGCTGCTGCAGCTGCCT | |
| | GAAGGGCTGTTGCAGCTGCGGCAGCTGCTGCAAGTTCGACGAG | |
| | GACGACAGCGAGCCCGTGCTGAAGGGCGTGAAGCTGCACTACA | |
| | CCTGATAATAGGCTGGAGCCTCGGTGGCCTAGCTTCTTGCCCCT | |
| | TGGGCCTCCCCCCAGCCCCTCCTCCCCTTCCTGCACCCGTACCC | |
| | CCGTGGTCTTTGAATAAAGTCTGAGTGGGCGGCAAAAAAAAA | |
| Nucleocapsid | ATGAGCGACAACGGCCCCCAGAACCAGAGGAACGCCCCCAGGA | 150 |
| | TCACCTTCGGCGGCCCCAGCGACAGCACCGGCAGCAACCAGAA | |
| | CGGCGAGAGGAGCGGCGCCAGGAGCAAGCAGAGGAGGCCCCA | |
| | GGGCCTGCCCAACAACACCGCCAGCTGGTTCACCGCCCTGACC | |
| | CAGCACGGCAAGGAGGACCTGAAGTTCCCCAGGGGCCAGGGC | |
| | GTGCCCATCAACACCAACAGCAGCCCCGACGACCAGATCGGCT | |
| | ACTACAGGAGGGCCACCAGGAGGATCAGGGGCGGCGACGGCA | |
| | AGATGAAGGACCTGAGCCCCAGGTGGTACTTCTACTACCTGGGC | |
| | ACCGGCCCCGAGGCCGGCCTGCCCTACGGCGCCAACAAGGAC | |
| | GGCATCATCTGGGTGGCCACCGAGGGCGCCCTGAACACCCCCA | |
| | AGGACCACATCGGCACCAGGAACCCCGCCAACAACGCCGCCAT | |
| | CGTGCTGCAGCTGCCCCAGGGCACCACCCTGCCCAAGGGCTTC | |
| | TACGCCGAGGGCAGCAGGGGCGGCAGCCAGGCCAGCAGCAGG | |
| | AGCAGCAGCAGGAGCAGGAACAGCAGCAGGAACAGCACCCCCG | |
| | GCAGCAGCAGGGGCACCAGCCCCGCCAGGATGGCCGGCAACG | |
| | GCGGCGACGCCGCCCTGGCCCTGCTGCTGCTGGACAGGCTGAA | |
| | CCAGCTGGAGAGCAAGATGAGCGGCAAGGGCCAGCAGCAGCAG | |
| | GGCCAGACCGTGACCAAGAAGAGCGCCGCCGAGGCCAGCAAGA | |
| | AGCCCAGGCAGAAGAGGACCGCCACCAAGGCCTACAACGTGAC | |
| | CCAGGCCTTCGGCAGGAGGGGCCCCGAGCAGACCCAGGGCAA | |
| | CTTCGGCGACCAGGAGCTGATCAGGCAGGGCACCGACTACAAG | |
| | CACTGGCCCCAGATCGCCCAGTTCGCCCCCAGCGCCAGCGCCT | |
| | TCTTCGGCATGAGCAGGATCGGCATGGAGGTGACCCCCAGCGG | |
| | CACCTGGCTGACCTACACCGGCGCCATCAAGCTGGACGACAAG | |
| | GACCCCAACTTCAAGGACCAGGTGATCCTGCTGAACAAGCACAT | |
| | CGACGCCTACAAGACCTTCCCCCCCACCGAGCCCAAGAAGGAC | |
| | AAGAAGAAGAAGGCCGACGAGACCCAGGCCCTGCCCCAGAGGC | |
| | AGAAGAAGCAGCAGACCGTGACCCTGCTGCCCGCCGCCGACCT | |
| | GGACGACTTCAGCAAGCAGCTGCAGCAGAGCATGAGCAGCGCC | |
| | GACAGCACCCAGGCC | |
| ORFtab (non- | CAAACCACTGAAACAGCWCACTCTTGTAATGTTAACCGCTTTAAT | 151 |
| annotated) | GTGGCTATTACAAGAGCAAAAATTGGCATTGTGCATAATGTCT | |
| | GACAGAGATCTTTATGACAAGCTGCAATTCACAAGTCTAGAAGTA | |
| | CCGCGTCGTAACGTGGCTACATTACAAGCGGAAAATGTAACTGG | |
| | ACTCTTTAAGGACTGTAGTAAGATCATAACTGGTCTTCATCCTAC | |
| | ACAAGCACCTACACACCTTAGTGTTGATACAAAATTCAAGACTGA | |
| | GGGACTATGTGTTGACATACCAGGCATWCCWAAGGACATGACC | |
| | TATMGWAGACTCATCTCYATGATGGGTTTCAAAATGAATTAYCAA | |
| | GTTAATGGTTACCCTAAYATGTTYATCACCCGYGARGAAGCCATM | |
| | MGMCAYGTWCGTGCATGGATTGGCTTTGATGTAGAGGGKTGTC | |
| | ATGCTACTAGGGATGCTGTCGGTACTAACCTACCTCTCCAGTTAG | |
| | GATTTTCTACAGGTGTTAACTTAGTAGCTGTACCAACTGGCTATG | |
| | TTGACACTGAAAACAATACAGAATTCACCAGAGTTAATGCAAAAC | |
| | CTCCACCAGGTGACCAATTTAAACATCTTATACCACTTATGTACA | |
| | AAGGTTTACCCTGGAACATAGTGCGTATCAAGATAGTACAAATGC | |
| | TCAGTGATACACTGAAAGGATTATCRGACAGAGTTGTGTTTGTCC | |
| | TATGGGCACATGGCTTTGAACTTACATCAATGAAGTACTTTGTCA | |
| | AGATTGGACCTGAAAGAACGTGTTGTCTGTGTGACAAACGTGCA | |
| | ACTTGTTTTTCTACTTCATCAGACAATTATGCCTGCTGGAACCATT | |
| | CTGTGGGTTTTGACTATGTCTATAATCCATTTATGATTGATGTCCA | |
| | GCAGTGGGGTTTTACAGGTAACCTTCAGAGTAATCACGATCAGC | |
| | ATTGCCAAGTGCATGGCAACGCTCATGTGGCTAGTTGTGATGCT | |
| | ATCATGACTAGATGTTTAGCAGTCCATGAGTGCTTTGTTAAGCGC | |
| | GTTGACTGGTCTGTTGAGTACCCAATTATAGGTGATGAACTGAAG | |
| | ATCAATGCCGCATGCAGAAAAGTGCAACATATGGTTGTAAAGTCT | |
| | GCATTGCTTGCTGACAAATTCCCAGTTCTTCATGACATTGGAAAC | |
| | CCAAAGGCTATCAAATGTGTCCCRCAGGCTGAAGTGGATTGGAA | |
| | GTTCTATGATGCTCAGCCCTGCAGTGACAAAGCTTATAAAATAAA | |
| | AGAACTCTTCTATTCTTATGCTACACATCATGATAAATTCATTGAT | |
| | GGTGTTTGTTTATTTTGGAATTGTAACGTTGATCGTTACCCTGCC | |
| | AATGCTATTGTRTGCAGGTTCGACACGAGAGTCTTGTCAAATTTG | |
| | AACTTGCCAGGTTGTGATGGTGGTAGTTTGTATGTAAATAAGCAT | |
| | GCATTCCACACTCCAGCTTTTGATAAAAGTGCATTTACTAATTTAA | |
| | AGCAATTGCCTTTCTTTTATTACTCTGACAGTCCCTGTGAGTCAC | |
| | ATGGCAAGCAGGTTGTTTCTGACATTGATTATGTACCACTCAAAT | |
| | CTGCTACRTGTATAACACGATGCAATTTGGGRGGTGCTGTTTGC | |
| | AGACATCATGCAAATGAGTACCGACAGTACTTGGATGCATACAAT | |
| | ATGATGATTTCTGCTGGCTTTAGCCTCTGGATTTACAAACAGTTT | |
| | GACACTTATAACCTGTGGAACACCTTTACCAGGTTACAGAGTTTA | |
| | GAAAATGTGGCTTACAATGTTGTTAACAAAGGACACTTCGATGGA | |
| | CAAGCTGGTGAAGCACCTGTTTCCGTCATTAATAATGTTGTTTAC | |
| | ACAAAGGTAGATGGTGTTGATGTAGAGATCTTTGAAAACAAGACA | |
| | ACACTTCCTGTTAATGTTGCATTTGAGCTTTGGGCTAAGCGTAAC | |
| | ATTAAACCAGTGCCAGAGATTAAGATACTCAATAATTTGGGTGTC | |
| | GATATCGCTGCTAATACTGTAATCTGGGACTACAAGAGAGAAGC | |
| | ACCAGCACATATGTCAACAATAGGTGTCTGCACAATGACTGACAT | |
| | TGCCAAGAAACCTACTGAGAGTGCTTGTTCCTCGCTTACTGTCTT | |
| | ATTTGATGGTAGAGTGGAAGGACAGGTAGACCTTTTTAGAAATGC | |
| | CCGTAATGGTGTTTTAATAACAGAAGGTTCAGTTAAAGGTTTAAT | |
| | ACCTTCAAAGGGACCAGCACAAGCTAGTGTCAATGGAGTCACAT | |
| | TAATTGGAGAATCAGTAAAAACACAGTTTAATTATTTTAAGAAAGT | |
| | AGATGGCATCATTCAACAGTTGCCTGAAACCTACTTTACTCAGAG | |
| | CCGAGACTTAGAGGATTTCAAGCCCAGATCACAAATGGAAACTG | |
| | ACTTTCTTGAGCTCGCTATGGATGAATTCATACAACGGTACAAGC | |
| | TTGAAGGCTATGCCTTCGAACATATCGTTTATGGAGATAGTC | |
| | ATGGACAGCTTGGTGGACTTCATCTAATGATTGGTCTAGCTAAGC | |
| | GCTCACAAGATTCACCACTTAAATTAGAGGATTTTATCCCTACGG | |
| | ACAGTACAGTGAAAAATTATTTCATAACAGATGCGCAAACAGGTT | |
| | CATCAAAATGCGTGTGCTCTGTTATTGATCTTCTGCTTGATGACT | |
| | TTGTTGAGATAATAAAGTCACAAGATTTATCAGTGGTTTCAAAGG | |
| | TGGTCAAAGTCACAATTGACTATGCTGAAATTTCATTCATGTTATG | |
| | GTGTAAGGATGGACATGTTGAAACCTTACCCAAAATTACAAGC | |
| | GAGTCAGGCGTGGCAACCAGGAGTTGCAATGCCTAACTTGTATA | |
| | AGATGCAGAGAATGCTTCTTGAAAAATGTGACCTTCAGAATTATG | |
| | GTGAAAATGCTGTCATACCAAARGGAATAATGATGAATGTCGCAA | |
| | AATATACTCAACTGTGTCAATATTTAAATACACTYACATTAGCYGT | |
| | GCCATATAATATGAGAGTTATCCAMTTGGTGCTGGCTCRGACAA | |
| | AGGAGTTGCACCCGGCACAGCTGTTCTCAGACAGTGGTTGCCAA | |
| | TTGGCACACTACTTGTTGATTCAGATCTTAACGACTTCGTCTCTG | |
| | ACGCTGATTCCACTCTAATTGGAGACTGTGCAACCGTACATACAG | |
| | CTAACAAATGGGATCTCATTATTAGCGATATGTATGATCCTAAAA | |
| | CCAAACACGTGACAAAGGAAAATGATTCAAAAGAAGGATTTTTCA | |
| | CTTACCTGTGTGGATTTATTAAACAAAAATTAGCCCTGGGAGGCT | |
| | CTGTGGCTGTAAAGATAACTGAGCATTCTTGGAATGCGGATCTCT | |
| | ACAAGCTCATGGGACATTTCTCATGGTGGACAGCTTTTGTTACAA | |
| | ATGTTAATGCATCTTCATCAGAAGCATTTTTAATTGGAGTTAACTA | |
| | TCTTGGTAAGCCAAAAGAACAAATTGATGGTTACACCATGCATGC | |
| | TAACTACATTCTGGAGGAATACAAACCCGATTCAATTGTCTTC | |
| | CTATTCACTTTTTGACATGAGTAAGTTCCCTCTTAAATTAAGGGGA | |
| | ACAGCTGTCATGTCTTTAAAGGAGAACCAAATCAATGAAATGATT | |
| | TATTCTCTACTTGAAAAAGGCAGACTTATCATTAGGGAAAACAAC | |
| | AGAGTTGTTGTCTCAAGTGATGTTCTTGTTAATAACTAAACGAAC | |
| | A | |
| Orfta/b | GACCAGGAACCCCGCCTGGAGGAAGGCCGTGTTCATCAGCCCC | 348 |
| | TACAACAGCCAGAACGCCGTGGCCAGCAAGATCCTGGGCCTGC | |
| | CCACCCAGACCGTGGACAGCAGCCAGGGCAGCGAGTACGACTA | |
| | CGTGATCTTCACCCAGACCACCGAGACCGCCCACAGCTGCAAC | |
| | GTGAACAGGTTCAACGTGGCCATCACCAGGGCCAAGGTGGGCA | |
| | TCCTGTGCATCATGAGCGACAGGGACCTGTACGACAAGCTGCAG | |
| | TTCACCAGCCTGGAGATCCCCAGGAGGAACGTGGCCACCCTGC | |
| | AGGCCGAGAACGTGACCGGCCTGTTCAAGGACTGCAGCAAGGT | |
| | GATCACCGGCCTGCACCCCACCCAGGCCCCCACCCACCTGAGC | |
| | GTGGACACCAAGTTCAAGACCGAGGGCCTGTGCGTGGACATCC | |
| | CCGGCATCCCCAAGGACATGACCTACAGGAGGCTGATCAGCAT | |
| | GATGGGCTTCAAGATGAACTACCAGGTGAACGGCTACCCCAACA | |
| | TGTTCATCACCAGGGAGGAGGCCATCAGGCACGTGAGGGCCTG | |
| | GATCGGCTTCGACGTGGAGGGCTGCCACGCCACCAGGGAGGCC | |
| | GTGGGCACCAACCTGCCCCTGCAGCTGGGCTTCAGCACCGGCG | |
| | TGAACCTGGTGGCCGTGCCCACCGGCTACGTGGACACCCCCAA | |
| | CAACACCGACTTCAGCAGGGTGAGCGCCAAGCCCCCCCCCGGC | |
| | GACCAGTTCAAGCACCTGATCCCCCTGATGTACAAGGGCCTGCC | |
| | CTGGAACGTGGTGAGGATCAAGATCGTGCAGATGCTGAGCGAC | |
| | ACCCTGAAGAACCTGAGCGACAGGGTGGTGTTCGTGCTGTGGG | |
| | CCCACGGCTTCGAGCTGACCAGCATGAAGTACTTCGTGAAGATC | |
| | GGCCCCGAGAGGACCTGCTGCCTGTGCGACAGGAGGGCCACCT | |
| | GCTTCAGCACCGCCAGCGACACCTACGCCTGCTGGCACCACAG | |
| | CATCGGCTTCGACTACGTGTACAACCCCTTCATGATCGACGTGC | |
| | AGCAGTGGGGCTTCACCGGCAACCTGCAGAGCAACCACGACCT | |
| | GTACTGCCAGGTGCACGGCAACGCCCACGTGGCCAGCTGCGAC | |
| | GCCATCATGACCAGGTGCCTGGCCGTGCACGAGTGCTTCGTGA | |
| | AGAGGGTGGACTGGACCATCGAGTACCCCATCATCGGCGACGA | |
| | GCTGAAGATCAACGCCGCCTGCAGGAAGGTGCAGCACATGGTG | |
| | GTGAAGGCCGCCCTGCTGGCCGACAAGTTCCCCGTGCTGCACG | |
| | ACATCGGCAACCCCAAGGCCATCAAGTGCGTGCCCCAGGCCGA | |
| | CGTGGAGTGGAAGTTCTACGACGCCCAGCCCTGCAGCGACAAG | |
| | GCCTACAAGATCGAGGAGCTGTTCTACAGCTACGCCACCCACAG | |
| | CGACAAGTTCACCGACGGCGTGTGCCTGTTCTGGAACTGCAACG | |
| | TGGACAGGTACCCCGCCAACAGCATCGTGTGCAGGTTCGACAC | |
| | CAGGGTGCTGAGCAACCTGAACCTGCCCGGCTGCGACGGCGGC | |
| | AGCCTGTACGTGAACAAGCACGCCTTCCACACCCCCGCCTTCGA | |
| | CAAGAGCGCCTTCGTGAACCTGAAGCAGCTGCCCTTCTTCTACT | |
| | ACAGCGACAGCCCCTGCGAGAGCCACGGCAAGCAGGTGGTGAG | |
| | CGACATCGACTACGTGCCCCTGAAGAGCGCCACCTGCATCACCA | |
| | GGTGCAACCTGGGCGGCGCCGTGTGCAGGCACCACGCCAACGA | |
| | GTACAGGCTGTACCTGGACGCCTACAACATGATGATCAGCGCCG | |
| | GCTTCAGCCTGTGGGTGTACAAGCAGTTCGACACCTACAACCTG | |
| | TGGAACACCTTCACCAGGCTGCAGAGCCTGGAGAACGTGGCCTT | |
| | CAACGTGGTGAACAAGGGCCACTTCGACGGCCAGCAGGGCGAG | |
| | GTGCCCGTGAGCATCATCAACAACACCGTGTACACCAAGGTGGA | |
| | CGGCGTGGACGTGGAGCTGTTCGAGAACAAGACCACCCTGCCC | |
| | GTGAACGTGGCCTTCGAGCTGTGGGCCAAGAGGAACATCAAGC | |
| | CCGTGCCCGAGGTGAAGATCCTGAACAACCTGGGCGTGGACAT | |
| | CGCCGCCAACACCGTGATCTGGGACTACAAGAGGGACGCCCCC | |
| | GCCCACATCAGCACCATCGGCGTGTGCAGCATGACCGACATCG | |
| | CCAAGAAGCCCACCGAGACCATCTGCGCCCCCCTGACCGTGTT | |
| | CTTCGACGGCAGGGTGGACGGCCAGGTGGACCTGTTCAGGAAC | |
| | GCCAGGAACGGCGTGCTGATCACCGAGGGCAGCGTGAAGGGC | |
| | CTGCAGCCCAGCGTGGGCCCCAAGCAGGCCAGCCTGAACGGC | |
| | GTGACCCTGATCGGCGAGGCCGTGAAGACCCAGTTCAACTACTA | |
| | CAAGAAGGTGGACGGCGTGGTGCAGCAGCTGCCCGAGACCTAC | |
| | TTCACCCAGAGCAGGAACCTGCAGGAGTTCAAGCCCAGGAGCC | |
| | AGATGGAGATCGACTTCCTGGAGCTGGCCATGGACGAGTTCATC | |
| | GAGAGGTACAAGCTGGAGGGCTACGCCTTCGAGCACATCGTGT | |
| | ACGGCGACTTCAGCCACAGCCAGCTGGGCGGCCTGCACCTGCT | |
| | GATCGGCCTGGCCAAGAGGTTCAAGGAGAGCCCCTTCGAGCTG | |
| | GAGGACTTCATCCCCATGGACAGCACCGTGAAGAACTACTTCAT | |
| | CACCGACGCCCAGACCGGCAGCAGCAAGTGCGTGTGCAGCGTG | |
| | ATCGACCTGCTGCTGGACGACTTCGTGGAGATCATCAAGAGCCA | |
| | GGACCTGAGCGTGGTGAGCAAGGTGGTGAAGGTGACCATCGAC | |
| | TACACCGAGATCAGCTTCATGCTGTGGTGCAAGGACGGCCACGT | |
| | GGAGACCTTCTACCCCAAGCTGCAGAGCAGCCAGGCCTGGCAG | |
| | CCCGGCGTGGCCATGCCCAACCTGTACAAGATGCAGAGGATGC | |
| | TGCTGGAGAAGTGCGACCTGCAGAACTACGGCGACAGCGCCAC | |
| | CCTGCCCAAGGGCATCATGATGAACGTGGCCAAGTACACCCAGC | |
| | TGTGCCAGTACCTGAACACCCTGACCCTGGCCGTGCCCTACAAC | |
| | ATGAGGGTGATCCACTTCGGCGCCGGCAGCGACAAGGGCGTGG | |
| | CCCCCGGCACCGCCGTGCTGAGGCAGTGGCTGCCCACCGGCA | |
| | CCCTGCTGGTGGACAGCGACCTGAACGACTTCGTGAGCGACGC | |
| | CGACAGCACCCTGATCGGCGACTGCGCCACCGTGCACACCGCC | |
| | AACAAGTGGGACCTGATCATCAGCGACATGTACGACCCCAAGAC | |
| | CAAGAACGTGACCAAGGAGAACGACAGCAAGGAGGGCTTCTTCA | |
| | CCTACATCTGCGGCTTCATCCAGCAGAAGCTGGCCCTGGGCGG | |
| | CAGCGTGGCCATCAAGATCACCGAGCACAGCTGGAACGCCGAC | |
| | CTGTACAAGCTGATGGGCCACTTCGCCTGGTGGACCGCCTTCGT | |
| | GACCAACGTGAACGCCAGCAGCAGCGAGGCCTTCCTGATCGGC | |
| | TGCAACTACCTGGGCAAGCCCAGGGAGCAGATCGACGGCTACG | |
| | TGATGCACGCCAACTACATCTTCTGGAGGAACACCAACCCCATC | |
| | CAGCTGAGCAGCTACAGCCTGTTCGACATGAGCAAGTTCCCCCT | |
| | GAAGCT | |
| ORF3a | ATGGACCTGTTCATGAGGATCTTCACCATCGGCACCGTGACCCT | 152 |
| | GAAGCAGGGCGAGATCAAGGACGCCACCCCCAGCGACTTCGTG | |
| | AGGGCCACCGCCACCATCCCCATCCAGGCCAGCCTGCCCTTCG | |
| | GCTGGCTGATCGTGGGCGTGGCCCTGCTGGCCGTGTTCCAGAG | |
| | CGCCAGCAAGATCATCACCCTGAAGAAGAGGTGGCAGCTGGCC | |
| | CTGAGCAAGGGCGTGCACTTCGTGTGCAACCTGCTGCTGCTGTT | |
| | CGTGACCGTGTACAGCCACCTGCTGCTGGTGGCCGCCGGCCTG | |
| | GAGGCCCCCTTCCTGTACCTGTACGCCCTGGTGTACTTCCTGCA | |
| | GAGCATCAACTTCGTGAGGATCATCATGAGGCTGTGGCTGTGCT | |
| | GGAAGTGCAGGAGCAAGAACCCCCTGCTGTACGACGCCAACTA | |
| | CTTCCTGTGCTGGCACACCAACTGCTACGACTACTGCATCCCCT | |
| | ACAACAGCGTGACCAGCAGCATCGTGATCACCAGCGGCGACGG | |
| | CACCACCAGCCCCATCAGCGAGCACGACTACCAGATCGGCGGC | |
| | TACACCGAGAAGTGGGAGAGCGGCGTGAAGGACTGCGTGGTGC | |
| | TGCACAGCTACTTCACCAGCGACTACTACCAGCTGTACAGCACC | |
| | CAGCTGAGCACCGACACCGGCGTGGAGCACGTGACCTTCTTCAT | |
| | CTACAACAAGATCGTGGACGAGCCCGAGGAGCACGTGCAGATC | |
| | CACACCATCGACGGCAGCAGCGGCGTGGTGAACCCCGTGATGG | |
| | AGCCCATCTACGACGAGCCCACCACCACCACCAGCGTGCCCCT | |
| | G | |
| Envelope(E) | ATGTACAGCTTCGTGAGCGAGGAGACCGGCACCCTGATCGTGAA | 153 |
| | CAGCGTGCTGCTGTTCCTGGCCTTCGTGGTGTTCCTGCTGGTGA | |
| | CCCTGGCCATCCTGACCGCCCTGAGGCTGTGCGCCTACTGCTG | |
| | CAACATCGTGAACGTGAGCCTGGTGAAGCCCAGCTTCTACGTGT | |
| | ACAGCAGGGTGAAGAACCTGAACAGCAGCAGGGTGCCCGACCT | |
| | GCTGGTG | |
| Membrane(M) | ATGGCCGACAGCAACGGCACCATCACCGTGGAGGAGCTGAAGA | 154 |
| | AGCTGCTGGAGCAGTGGAACCTGGTGATCGGCTTCCTGTTCCTG | |
| | ACCTGGATCTGCCTGCTGCAGTTCGCCTACGCCAACAGGAACAG | |
| | GTTCCTGTACATCATCAAGCTGATCTTCCTGTGGCTGCTGTGGC | |
| | CCGTGACCCTGGCCTGCTTCGTGCTGGCCGCCGTGTACAGGAT | |
| | CAACTGGATCACCGGCGGCATCGCCATCGCCATGGCCTGCCTG | |
| | GTGGGCCTGATGTGGCTGAGCTACTTCATCGCCAGCTTCAGGCT | |
| | GTTCGCCAGGACCAGGAGCATGTGGAGCTTCAACCCCGAGACC | |
| | AACATCCTGCTGAACGTGCCCCTGCACGGCACCATCCTGACCAG | |
| | GCCCCTGCTGGAGAGCGAGCTGGTGATCGGCGCCGTGATCCTG | |
| | AGGGGCCACCTGAGGATCGCCGGCCACCACCTGGGCAGGTGC | |
| | GACATCAAGGACCTGCCCAAGGAGATCACCGTGGCCACCAGCA | |
| | GGACCCTGAGCTACTACAAGCTGGGCGCCAGCCAGAGGGTGGC | |
| | CGGCGACAGCGGCTTCGCCGCCTACAGCAGGTACAGGATCGGC | |
| | AACTACAAGCTGAACACCGACCACAGCAGCAGCAGCGACAACAT | |
| | CGCCCTGCTGGTGCAG | |
| ORFS | ATGTTCCACCTGGTGGACTTCCAGGTGACCATCGCCGAGATCCT | 155 |
| | GCTGATCATCATGAGGACCTTCAAGGTGAGCATCTGGAACCTGG | |
| | ACTACATCATCAACCTGATCATCAAGAACCTGAGCAAGAGCCTGA | |
| | CCGAGAACAAGTACAGCCAGCTGGACGAGGAGCAGCCCATGGA | |
| | GATCGAC | |
| ORF7a | ATGAAGATCATCCTGTTCCTGGCCCTGATCACCCTGGCCACCTG | 156 |
| | CGAGCTGTACCACTACCAGGAGTGCGTGAGGGGCACCACCGTG | |
| NSP1 | ATGGAGAGCCTGGTGCCCGGCTTCAACGAGAAGACCCACGTGC | 349 |
| | AGCTGAGCCTGCCCGTGCTGCAGGTGAGGGACGTGCTGGTGAG | |
| | GGGCTTCGGCGACAGCGTGGAGGAGGTGCTGAGCGAGGCCAG | |
| | GCAGCACCTGAAGGACGGCACCTGCGGCCTGGTGGAGGTGGA | |
| | GAAGGGCGTGCTGCCCCAGCTGGAGCAGCCCTACGTGTTCATC | |
| | AAGAGGAGCGACGCCAGGACCGCCCCCCACGGCCACGTGATG | |
| | GTGGAGCTGGTGGCCGAGCTGGAGGGCATCCAGTACGGCAGGA | |
| | GCGGCGAGACCCTGGGCGTGCTGGTGCCCCACGTGGGCGAGA | |
| | TCCCCGTGGCCTACAGGAAGGTGCTGCTGAGGAAGAACGGCAA | |
| | CAAGGGCGCCGGCGGCCACAGCTACGGCGCCGACCTGAAGAG | |
| | CTTCGACCTGGGCGACGAGCTGGGCACCGACCCCTACGAGGAC | |
| | TTCCAGGAGAACTGGAACACCAAGCACAGCAGCGGCGTGACCA | |
| | GGGAGCTGATGAGGGAGCTGAACGGCGGC | |
| NSP2 | GCCTACACCAGGTACGTGGACAACAACTTCTGCGGCCCCGACG | 350 |
| | GCTACCCCCTGGAGTGCATCAAGGACCTGCTGGCCAGGGCCGG | |
| | CAAGGCCAGCTGCACCCTGAGCGAGCAGCTGGACTTCATCGAC | |
| | ACCAAGAGGGGCGTGTACTGCTGCAGGGAGCACGAGCACGAGA | |
| | TCGCCTGGTACACCGAGAGGAGCGAGAAGAGCTACGAGCTGCA | |
| | GACCCCCTTCGAGATCAAGCTGGCCAAGAAGTTCGACACCTTCA | |
| | ACGGCGAGTGCCCCAACTTCGTGTTCCCCCTGAACAGCATCATC | |
| | AAGACCATCCAGCCCAGGGTGGAGAAGAAGAAGCTGGACGGCT | |
| | TCATGGGCAGGATCAGGAGCGTGTACCCCGTGGCCAGCCCCAA | |
| | CGAGTGCAACCAGATGTGCCTGAGCACCCTGATGAAGTGCGAC | |
| | CACTGCGGCGAGACCAGCTGGCAGACCGGCGACTTCGTGAAGG | |
| | CCACCTGCGAGTTCTGCGGCACCGAGAACCTGACCAAGGAGGG | |
| | CGCCACCACCTGCGGCTACCTGCCCCAGAACGCCGTGGTGAAG | |
| | ATCTACTGCCCCGCCTGCCACAACAGCGAGGTGGGCCCCGAGC | |
| | ACAGCCTGGCCGAGTACCACAACGAGAGCGGCCTGAAGACCAT | |
| | CCTGAGGAAGGGCGGCAGGACCATCGCCTTCGGCGGCTGCGTG | |
| | TTCAGCTACGTGGGCTGCCACAACAAGTGCGCCTACTGGGTGCC | |
| | CAGGGCCAGCGCCAACATCGGCTGCAACCACACCGGCGTGGTG | |
| | GGCGAGGGCAGCGAGGGCCTGAACGACAACCTGCTGGAGATCC | |
| | TGCAGAAGGAGAAGGTGAACATCAACATCGTGGGCGACTTCAAG | |
| | CTGAACGAGGAGATCGCCATCATCCTGGCCAGCTTCAGCGCCA | |
| | GCACCAGCGCCTTCGTGGAGACCGTGAAGGGCCTGGACTACAA | |
| | GGCCTTCAAGCAGATCGTGGAGAGCTGCGGCAACTTCAAGGTG | |
| | ACCAAGGGCAAGGCCAAGAAGGGCGCCTGGAACATCGGCGAGC | |
| | AGAAGAGCATCCTGAGCCCCCTGTACGCCTTCGCCAGCGAGGC | |
| | CGCCAGGGTGGTGAGGAGCATCTTCAGCAGGACCCTGGAGACC | |
| | GCCCAGAACAGCGTGAGGGTGCTGCAGAAGGCCGCCATCACCA | |
| | TCCTGGACGGCATCAGCCAGTACAGCCTGAGGCTGATCGACGC | |
| | CATGATGTTCACCAGCGACCTGGCCACCAACAACCTGGTGGTGA | |
| | TGGCCTACATCACCGGCGGCGTGGTGCAGCTGACCAGCCAGTG | |
| | GCTGACCAACATCTTCGGCACCGTGTACGAGAAGCTGAAGCCCG | |
| | TGCTGGACTGGCTGGAGGAGAAGTTCAAGGAGGGCGTGGAGTT | |
| | CCTGAGGGACGGCTGGGAGATCGTGAAGTTCATCAGCACCTGC | |
| | GCCTGCGAGATCGTGGGCGGCCAGATCGTGACCTGCGCCAAGG | |
| | AGATCAAGGAGAGCGTGCAGACCTTCTTCAAGCTGGTGAACAAG | |
| | TTCCTGGCCCTGTGCGCCGACAGCATCATCATCGGCGGCGCCA | |
| | AGCTGAAGGCCCTGAACCTGGGCGAGACCTTCGTGACCCACAG | |
| | CAAGGGCCTGTACAGGAAGTGCGTGAAGAGCAGGGAGGAGACC | |
| | GGCCTGCTGATGCCCCTGAAGGCCCCCAAGGAGATCATCTTCCT | |
| | GGAGGGCGAGACCCTGCCCACCGAGGTGCTGACCGAGGAGGT | |
| | GGTGCTGAAGACCGGCGACCTGCAGCCCCTGGAGCAGCCCACC | |
| | AGCGAGGCCGTGGAGGCCCCCCTGGTGGGCACCCCCGTGTGC | |
| | ATCAACGGCCTGATGCTGCTGGAGATCAAGGACACCGAGAAGTA | |
| | CTGCGCCCTGGCCCCCAACATGATGGTGACCAACAACACCTTCA | |
| | CCCTGAAGGGCGGC | |
| Nsp3 | GCCCCCACCAAGGTGACCTTCGGCGACGACACCGTGATCGAGG | 157 |
| | TGCAGGGCTACAAGAGCGTGAACATCACCTTCGAGCTGGACGA | |
| | GAGGATCGACAAGGTGCTGAACGAGAAGTGCAGCGCCTACACC | |
| | GTGGAGCTGGGCACCGAGGTGAACGAGTTCGCCTGCGTGGTGG | |
| | CCGACGCCGTGATCAAGACCCTGCAGCCCGTGAGCGAGCTGCT | |
| | GACCCCCCTGGGCATCGACCTGGACGAGTGGAGCATGGCCACC | |
| | TACTACCTGTTCGACGAGAGCGGCGAGTTCAAGCTGGCCAGCCA | |
| | CATGTACTGCAGCTTCTACCCCCCCGACGAGGACGAGGAGGAG | |
| | GGCGACTGCGAGGAGGAGGAGTTCGAGCCCAGCACCCAGTACG | |
| | AGTACGGCACCGAGGACGACTACCAGGGCAAGCCCCTGGAGTT | |
| | CGGCGCCACCAGCGCCGCCCTGCAGCCCGAGGAGGAGCAGGA | |
| | GGAGGACTGGCTGGACGACGACAGCCAGCAGACCGTGGGCCA | |
| | GCAGGACGGCAGCGAGGACAACCAGACCACCACCATCCAGACC | |
| | ATCGTGGAGGTGCAGCCCCAGCTGGAGATGGAGCTGACCCCCG | |
| | TGGTGCAGACCATCGAGGTGAACAGCTTCAGCGGCTACCTGAAG | |
| | CTGACCGACAACGTGTACATCAAGAACGCCGACATCGTGGAGGA | |
| | GGCCAAGAAGGTGAAGCCCACCGTGGTGGTGAACGCCGCCAAC | |
| | GTGTACCTGAAGCACGGCGGCGGCGTGGCCGGCGCCCTGAACA | |
| | AGGCCACCAACAACGCCATGCAGGTGGAGAGCGACGACTACAT | |
| | CGCCACCAACGGCCCCCTGAAGGTGGGCGGCAGCTGCGTGCTG | |
| | AGCGGCCACAACCTGGCCAAGCACTGCCTGCACGTGGTGGGCC | |
| | CCAACGTGAACAAGGGCGAGGACATCCAGCTGCTGAAGAGCGC | |
| | CTACGAGAACTTCAACCAGCACGAGGTGCTGCTGGCCCCCCTG | |
| | CTGAGCGCCGGCATCTTCGGCGCCGACCCCATCCACAGCCTGA | |
| | GGGTGTGCGTGGACACCGTGAGGACCAACGTGTACCTGGCCGT | |
| | GTTCGACAAGAACCTGTACGACAAGCTGGTGAGCAGCTTCCTGG | |
| | AGATGAAGAGCGAGAAGCAGGTGGAGCAGAAGATCGCCGAGAT | |
| | CCCCAAGGAGGAGGTGAAGCCCTTCATCACCGAGAGCAAGCCC | |
| | AGCGTGGAGCAGAGGAAGCAGGACGACAAGAAGATCAAGGCCT | |
| | GCGTGGAGGAGGTGACCACCACCCTGGAGGAGACCAAGTTCCT | |
| | GACCGAGAACCTGCTGCTGTACATCGACATCAACGGCAACCTGC | |
| | ACCCCGACAGCGCCACCCTGGTGAGCGACATCGACATCACCTTC | |
| | CTGAAGAAGGACGCCCCCTACATCGTGGGCGACGTGGTGCAGG | |
| | AGGGCGTGCTGACCGCCGTGGTGATCCCCACCAAGAAGGCCGG | |
| | CGGCACCACCGAGATGCTGGCCAAGGCCCTGAGGAAGGTGCCC | |
| | ACCGACAACTACATCACCACCTACCCCGGCCAGGGCCTGAACG | |
| | GCTACACCGTGGAGGAGGCCAAGACCGTGCTGAAGAAGTGCAA | |
| | GAGCGCCTTCTACATCCTGCCCAGCATCATCAGCAACGAGAAGC | |
| | AGGAGATCCTGGGCACCGTGAGCTGGAACCTGAGGGAGATGCT | |
| | GGCCCACGCCGAGGAGACCAGGAAGCTGATGCCCGTGTGCGTG | |
| | GAGACCAAGGCCATCGTGAGCACCATCCAGAGGAAGTACAAGG | |
| | GCATCAAGATCCAGGAGGGCGTGGTGGACTACGGCGCCAGGTT | |
| | CTACTTCTACACCAGCAAGACCACCGTGGCCAGCCTGATCAACA | |
| | CCCTGAACGACCTGAACGAGACCCTGGTGACCATGCCCCTGGG | |
| | CTACGTGACCCACGGCCTGAACCTGGAGGAGGCCGCCAGGTAC | |
| | ATGAGGAGCCTGAAGGTGCCCGCCACCGTGAGCGTGAGCAGCC | |
| | CCGACGCCGTGACCGCCTACAACGGCTACCTGACCAGCAGCAG | |
| | CAAGACCCCCGAGGAGCACTTCATCGAGACCATCAGCCTGGCC | |
| | GGCAGCTACAAGGACTGGAGCTACAGCGGCCAGAGCACCCAGC | |
| | TGGGCATCGAGTTCCTGAAGAGGGGCGACAAGAGCGTGTACTA | |
| | CACCAGCAACCCCACCACCTTCCACCTGGACGGCGAGGTGATC | |
| | ACCTTCGACAACCTGAAGACCCTGCTGAGCCTGAGGGAGGTGA | |
| | GGACCATCAAGGTGTTCACCACCGTGGACAACATCAACCTGCAC | |
| Nsp5 | AGCGGCTTCAGGAAGATGGCCTTCCCCAGCGGCAAGGTGGAGG | 158 |
| | GCTGCATGGTGCAGGTGACCTGCGGCACCACCACCCTGAACGG | |
| | CCTGTGGCTGGACGACGTGGTGTACTGCCCCAGGCACGTGATC | |
| | TGCACCAGCGAGGACATGCTGAACCCCAACTACGAGGACCTGCT | |
| | GATCAGGAAGAGCAACCACAACTTCCTGGTGCAGGCCGGCAAC | |
| | GTGCAGCTGAGGGTGATCGGCCACAGCATGCAGAACTGCGTGC | |
| | TGAAGCTGAAGGTGGACACCGCCAACCCCAAGACCCCCAAGTA | |
| | CAAGTTCGTGAGGATCCAGCCCGGCCAGACCTTCAGCGTGCTG | |
| | GCCTGCTACAACGGCAGCCCCAGCGGCGTGTACCAGTGCGCCA | |
| | TGAGGCCCAACTTCACCATCAAGGGCAGCTTCCTGAACGGCAGC | |
| | TGCGGCAGCGTGGGCTTCAACATCGACTACGACTGCGTGAGCTT | |
| | CTGCTACATGCACCACATGGAGCTGCCCACCGGCGTGCACGCC | |
| | GGCACCGACCTGGAGGGCAACTTCTACGGCCCCTTCGTGGACA | |
| | GGCAGACCGCCCAGGCCGCCGGCACCGACACCACCATCACCGT | |
| | GAACGTGCTGGCCTGGCTGTACGCCGCCGTGATCAACGGCGAC | |
| | AGGTGGTTCCTGAACAGGTTCACCACCACCCTGAACGACTTCAA | |
| | CCTGGTGGCCATGAAGTACAACTACGAGCCCCTGACCCAGGAC | |
| | CACGTGGACATCCTGGGCCCCCTGAGCGCCCAGACCGGCATCG | |
| | CCGTGCTGGACATGTGCGCCAGCCTGAAGGAGCTGCTGCAGAA | |
| | CGGCATGAACGGCAGGACCATCCTGGGCAGCGCCCTGCTGGAG | |
| | GACGAGTTCACCCCCTTCGACGTGGTGAGGCAGTGCAGCGGCG | |
| | TGACCTTCCAG | |
| NSP6 | AGCGCCGTGAAGAGGACCATCAAGGGCACCCACCACTGGCTGC | 351 |
| | TGCTGACCATCCTGACCAGCCTGCTGGTGCTGGTGCAGAGCAC | |
| | CCAGTGGAGCCTGTTCTTCTTCCTGTACGAGAACGCCTTCCTGC | |
| | CCTTCGCCATGGGCATCATCGCCATGAGCGCCTTCGCCATGATG | |
| | TTCGTGAAGCACAAGCACGCCTTCCTGTGCCTGTTCCTGCTGCC | |
| | CAGCCTGGCCACCGTGGCCTACTTCAACATGGTGTACATGCCCG | |
| | CCAGCTGGGTGATGAGGATCATGACCTGGCTGGACATGGTGGA | |
| | CACCAGCCTGAGCGGCTTCAAGCTGAAGGACTGCGTGATGTAC | |
| | GCCAGCGCCGTGGTGCTGCTGATCCTGATGACCGCCAGGACCG | |
| | TGTACGACGACGGCGCCAGGAGGGTGTGGACCCTGATGAACGT | |
| | GCTGACCCTGGTGTACAAGGTGTACTACGGCAACGCCCTGGAC | |
| | CAGGCCATCAGCATGTGGGCCCTGATCATCAGCGTGACCAGCAA | |
| | CTACAGCGGCGTGGTGACCACCGTGATGTTCCTGGCCAGGGGC | |
| | ATCGTGTTCATGTGCGTGGAGTACTGCCCCATCTTCTTCATCACC | |
| | GGCAACACCCTGCAGTGCATCATGCTGGTGTACTGCTTCCTGGG | |
| | CTACTTCTGCACCTGCTACTTCGGCCTGTTCTGCCTGCTGAACA | |
| | GGTACTTCAGGCTGACCCTGGGCGTGTACGACTACCTGGTGAG | |
| | CACCCAGGAGTTCAGGTACATGAACAGCCAGGGCCTGCTGCCC | |
| | CCCAAGAACAGCATCGACGCCTTCAAGCTGAACATCAAGCTGCT | |
| | GGGCGTGGGCGGCAAGCCCTGCATCAAGGTGGCCACCGTGCA | |
| | G | |
| NSP7 | AGCAAGATGAGCGACGTGAAGTGCACCAGCGTGGTGCTGCTGA | 352 |
| | GCGTGCTGCAGCAGCTGAGGGTGGAGAGCAGCAGCAAGCTGTG | |
| | GGCCCAGTGCGTGCAGCTGCACAACGACATCCTGCTGGCCAAG | |
| | GACACCACCGAGGCCTTCGAGAAGATGGTGAGCCTGCTGAGCG | |
| | TGCTGCTGAGCATGCAGGGCGCCGTGGACATCAACAAGCTGTG | |
| | CGAGGAGATGCTGGACAACAGGGCCACCCTGCAG | |
| NSP8 | GCCATCGCCAGCGAGTTCAGCAGCCTGCCCAGCTACGCCGCCT | 353 |
| | TCGCCACCGCCCAGGAGGCCTACGAGCAGGCCGTGGCCAACG | |
| | GCGACAGCGAGGTGGTGCTGAAGAAGCTGAAGAAGAGCCTGAA | |
| | CGTGGCCAAGAGCGAGTTCGACAGGGACGCCGCCATGCAGAGG | |
| | AAGCTGGAGAAGATGGCCGACCAGGCCATGACCCAGATGTACA | |
| | AGCAGGCCAGGAGCGAGGACAAGAGGGCCAAGGTGACCAGCG | |
| | CCATGCAGACCATGCTGTTCACCATGCTGAGGAAGCTGGACAAC | |
| | GACGCCCTGAACAACATCATCAACAACGCCAGGGACGGCTGCG | |
| | TGCCCCTGAACATCATCCCCCTGACCACCGCCGCCAAGCTGATG | |
| | GTGGTGATCCCCGACTACAACACCTACAAGAACACCTGCGACGG | |
| | CACCACCTTCACCTACGCCAGCGCCCTGTGGGAGATCCAGCAG | |
| | GTGGTGGACGCCGACAGCAAGATCGTGCAGCTGAGCGAGATCA | |
| | GCATGGACAACAGCCCCAACCTGGCCTGGCCCCTGATCGTGAC | |
| | CGCCCTGAGGGCCAACAGCGCCGTGAAGCTGCA | |
| NSP9 | AACAACGAGCTGAGCCCCGTGGCCCTGAGGCAGATGAGCTGCG | 354 |
| | CCGCCGGCACCACCCAGACCGCCTGCACCGACGACAACGCCCT | |
| | GGCCTACTACAACACCACCAAGGGCGGCAGGTTCGTGCTGGCC | |
| | CTGCTGAGCGACCTGCAGGACCTGAAGTGGGCCAGGTTCCCCA | |
| | AGAGCGACGGCACCGGCACCATCTACACCGAGCTGGAGCCCCC | |
| | CTGCAGGTTCGTGACCGACACCCCCAAGGGCCCCAAGGTGAAG | |
| | TACCTGTACTTCATCAAGGGCCTGAACAACCTGAACAGGGGCAT | |
| | GGTGCTGGGCAGCCTGGCCGCCACCGTGAGGCTGCAG | |
| NSP10 | GCCGGCAACGCCACCGAGGTGCCCGCCAACAGCACCGTGCTGA | 355 |
| | GCTTCTGCGCCTTCGCCGTGGACGCCGCCAAGGCCTACAAGGA | |
| | CTACCTGGCCAGCGGCGGCCAGCCCATCACCAACTGCGTGAAG | |
| | ATGCTGTGCACCCACACCGGCACCGGCCAGGCCATCACCGTGA | |
| | CCCCCGAGGCCAACATGGACCAGGAGAGCTTCGGCGGCGCCAG | |
| | CTGCTGCCTGTACTGCAGGTGCCACATCGACCACCCCAACCCCA | |
| | AGGGCTTCTGCGACCTGAAGGGCAAGTACGTGCAGATCCCCAC | |
| | CACCTGCGCCAACGACCCCGTGGGCTTCACCCTGAAGAACACC | |
| | GTGTGCACCGTGTGCGGCATGTGGAAGGGCTACGGCTGCAGCT | |
| | GCGACCAGCTGAGGGAGCCCATGCTGCAG | |
| Nsp | AGCGCCGACGCCCAGAGCTTCCTGAACAGGGTGTGCGGCGTGA | 159 |
| | GCGCCGCCAGGCTGACCCCCTGCGGCACCGGCACCAGCACCG | |
| | ACGTGGTGTACAGGGCCTTCGACATCTACAACGACAAGGTGGCC | |
| | GGCTTCGCCAAGTTCCTGAAGACCAACTGCTGCAGGTTCCAGGA | |
| | GAAGGACGAGGACGACAACCTGATCGACAGCTACTTCGTGGTGA | |
| | AGAGGCACACCTTCAGCAACTACCAGCACGAGGAGACCATCTAC | |
| | AACCTGCTGAAGGACTGCCCCGCCGTGGCCAAGCACGACTTCTT | |
| | CAAGTTCAGGATCGACGGCGACATGGTGCCCCACATCAGCAGG | |
| | CAGAGGCTGACCAAGTACACCATGGCCGACCTGGTGTACGCCC | |
| | TGAGGCACTTCGACGAGGGCAACTGCGACACCCTGAAGGAGAT | |
| | CCTGGTGACCTACAACTGCTGCGACGACGACTACTTCAACAAGA | |
| | AGGACTGGTACGACTTCGTGGAGAACCCCGACATCCTGAGGGT | |
| | GTACGCCAACCTGGGCGAGAGGGTGAGGCAGGCCCTGCTGAAG | |
| | ACCGTGCAGTTCTGCGACGCCATGAGGAACGCCGGCATCGTGG | |
| | GCGTGCTGACCCTGGACAACCAGGACCTGAACGGCAACTGGTA | |
| | CGACTTCGGCGACTTCATCCAGACCACCCCCGGCAGCGGCGTG | |
| | CCCGTGGTGGACAGCTACTACAGCCTGCTGATGCCCATCCTGAC | |
| | CCTGACCAGGGCCCTGACCGCCGAGAGCCACGTGGACACCGAC | |
| | CTGACCAAGCCCTACATCAAGTGGGACCTGCTGAAGTACGACTT | |
| | CACCGAGGAGAGGCTGAAGCTGTTCGACAGGTACTTCAAGTACT | |
| | GGGACCAGACCTACCACCCCAACTGCGTGAACTGCCTGGACGA | |
| | CAGGTGCATCCTGCACTGCGCCAACTTCAACGTGCTGTTCAGCA | |
| | CCGTGTTCCCCCCCACCAGCTTCGGCCCCCTGGTGAGGAAGAT | |
| | CTTCGTGGACGGCGTGCCCTTCGTGGTGAGCACCGGCTACCAC | |
| | TTCAGGGAGCTGGGCGTGGTGCACAACCAGGACGTGAACCTGC | |
| | ACAGCAGCAGGCTGAGCTTCAAGGAGCTGCTGGTGTACGCCGC | |
| | CGACCCCGCCATGCACGCCGCCAGCGGCAACCTGCTGCTGGAC | |
| | AAGAGGACCACCTGCTTCAGCGTGGCCGCCCTGACCAACAACG | |
| | TGGCCTTCCAGACCGTGAAGCCCGGCAACTTCAACAAGGACTTC | |
| | TACGACTTCGCCGTGAGCAAGGGCTTCTTCAAGGAGGGCAGCA | |
| | GCGTGGAGCTGAAGCACTTCTTCTTCGCCCAGGACGGCAACGC | |
| | CGCCATCAGCGACTACGACTACTACAGGTACAACCTGCCCACCA | |
| | TGTGCGACATCAGGCAGCTGCTGTTCGTGGTGGAGGTGGTGGA | |
| | CAAGTACTTCGACTGCTACGACGGCGGCTGCATCAACGCCAACC | |
| | AGGTGATCGTGAACAACCTGGACAAGAGCGCCGGCTTCCCCTTC | |
| | AACAAGTGGGGCAAGGCCAGGCTGTACTACGACAGCATGAGCT | |
| | ACGAGGACCAGGACGCCCTGTTCGCCTACACCAAGAGGAACGT | |
| | GATCCCCACCATCACCCAGATGAACCTGAAGTACGCCATCAGCG | |
| | CCAAGAACAGGGCCAGGACCGTGGCCGGCGTGAGCATCTGCAG | |
| | CACCATGACCAACAGGCAGTTCCACCAGAAGCTGCTGAAGAGCA | |
| | TCGCCGCCACCAGGGGCGCCACCGTGGTGATCGGCACCAGCAA | |
| | GTTCTACGGCGGCTGGCACAACATGCTGAAGACCGTGTACAGC | |
| | GACGTGGAGAACCCCCACCTGATGGGCTGGGACTACCCCAAGT | |
| | GCGACAGGGCCATGCCCAACATGCTGAGGATCATGGCCAGCCT | |
| | GGTGCTGGCCAGGAAGCACACCACCTGCTGCAGCCTGAGCCAC | |
| | AGGTTCTACAGGCTGGCCAACGAGTGCGCCCAGGTGCTGAGCG | |
| | AGATGGTGATGTGCGGCGGCAGCCTGTACGTGAAGCCCGGCGG | |
| | CACCAGCAGCGGCGACGCCACCACCGCCTACGCCAACAGCGTG | |
| | TTCAACATCTGCCAGGCCGTGACCGCCAACGTGAACGCCCTGCT | |
| | GAGCACCGACGGCAACAAGATCGCCGACAAGTACGTGAGGAAC | |
| | CTGCAGCACAGGCTGTACGAGTGCCTGTACAGGAACAGGGACG | |
| | TGGACACCGACTTCGTGAACGAGTTCTACGCCTACCTGAGGAAG | |
| | CACTTCAGCATGATGATCCTGAGCGACGACGCCGTGGTGTGCTT | |
| | CAACAGCACCTACGCCAGCCAGGGCCTGGTGGCCAGCATCAAG | |
| | AACTTCAAGAGCGTGCTGTACTACCAGAACAACGTGTTCATGAG | |
| | CGAGGCCAAGTGCTGGACCGAGACCGACCTGACCAAGGGCCCC | |
| | CACGAGTTCTGCAGCCAGCACACCATGCTGGTGAAGCAGGGCG | |
| | ACGACTACGTGTACCTGCCCTACCCCGACCCCAGCAGGATCCTG | |
| | GGCGCCGGCTGCTTCGTGGACGACATCGTGAAGACCGACGGCA | |
| | CCCTGATGATCGAGAGGTTCGTGAGCCTGGCCATCGACGCCTAC | |
| | CCCCTGACCAAGCACCCCAACCAGGAGTACGCCGACGTGTTCC | |
| | ACCTGTACCTGCAGTACATCAGGAAGCTGCACGACGAGCTGACC | |
| | GGCCACATGCTGGACATGTACAGCGTGATGCTGACCAACGACAA | |
| | CACCAGCAGGTACTGGGAGCCCGAGTTCTACGAGGCCATGTAC | |
| | ACCCCCCACACCGTGCTGCAG | |
| NSP13 | GCCGTGGGCGCCTGCGTGCTGTGCAACAGCCAGACCAGCCTGA | 356 |
| | GGTGCGGCGCCTGCATCAGGAGGCCCTTCCTGTGCTGCAAGTG | |
| | CTGCTACGACCACGTGATCAGCACCAGCCACAAGCTGGTGCTGA | |
| | GCGTGAACCCCTACGTGTGCAACGCCCCCGGCTGCGACGTGAC | |
| | CGACGTGACCCAGCTGTACCTGGGCGGCATGAGCTACTACTGC | |
| | AAGAGCCACAAGCCCCCCATCAGCTTCCCCCTGTGCGCCAACG | |
| | GCCAGGTGTTCGGCCTGTACAAGAACACCTGCGTGGGCAGCGA | |
| | CAACGTGACCGACTTCAACGCCATCGCCACCTGCGACTGGACCA | |
| | ACGCCGGCGACTACATCCTGGCCAACACCTGCACCGAGAGGCT | |
| | GAAGCTGTTCGCCGCCGAGACCCTGAAGGCCACCGAGGAGACC | |
| | TTCAAGCTGAGCTACGGCATCGCCACCGTGAGGGAGGTGCTGA | |
| | GCGACAGGGAGCTGCACCTGAGCTGGGAGGTGGGCAAGCCCA | |
| | GGCCCCCCCTGAACAGGAACTACGTGTTCACCGGCTACAGGGT | |
| | GACCAAGAACAGCAAGGTGCAGATCGGCGAGTACACCTTCGAG | |
| | AAGGGCGACTACGGCGACGCCGTGGTGTACAGGGGCACCACCA | |
| | CCTACAAGCTGAACGTGGGCGACTACTTCGTGCTGACCAGCCAC | |
| | ACCGTGATGCCCCTGAGCGCCCCCACCCTGGTGCCCCAGGAGC | |
| | ACTACGTGAGGATCACCGGCCTGTACCCCACCCTGAACATCAGC | |
| | GACGAGTTCAGCAGCAACGTGGCCAACTACCAGAAGGTGGGCA | |
| | TGCAGAAGTACAGCACCCTGCAGGGCCCCCCCGGCACCGGCAA | |
| | GAGCCACTTCGCCATCGGCCTGGCCCTGTACTACCCCAGCGCC | |
| | AGGATCGTGTACACCGCCTGCAGCCACGCCGCCGTGGACGCCC | |
| | TGTGCGAGAAGGCCCTGAAGTACCTGCCCATCGACAAGTGCAG | |
| | CAGGATCATCCCCGCCAGGGCCAGGGTGGAGTGCTTCGACAAG | |
| | TTCAAGGTGAACAGCACCCTGGAGCAGTACGTGTTCTGCACCGT | |
| | GAACGCCCTGCCCGAGACCACCGCCGACATCGTGGTGTTCGAC | |
| | GAGATCAGCATGGCCACCAACTACGACCTGAGCGTGGTGAACG | |
| | CCAGGCTGAGGGCCAAGCACTACGTGTACATCGGCGACCCCGC | |
| | CCAGCTGCCCGCCCCCAGGACCCTGCTGACCAAGGGCACCCTG | |
| | GAGCCCGAGTACTTCAACAGCGTGTGCAGGCTGATGAAGACCAT | |
| | CGGCCCCGACATGTTCCTGGGCACCTGCAGGAGGTGCCCCGCC | |
| | GAGATCGTGGACACCGTGAGCGCCCTGGTGTACGACAACAAGC | |
| | TGAAGGCCCACAAGGACAAGAGCGCCCAGTGCTTCAAGATGTTC | |
| | TACAAGGGCGTGATCACCCACGACGTGAGCAGCGCCATCAACA | |
| | GGCCCCAGATCGGCGTGGTGAGGGAGTTCCTGACCAGGAACCC | |
| | CGCCTGGAGGAAGGCCGTGTTCATCAGCCCCTACAACAGCCAG | |
| | AACGCCGTGGCCAGCAAGATCCTGGGCCTGCCCACCCAGACCG | |
| | TGGACAGCAGCCAGGGCAGCGAGTACGACTACGTGATCTTCAC | |
| | CCAGACCACCGAGACCGCCCACAGCTGCAACGTGAACAGGTTC | |
| | AACGTGGCCATCACCAGGGCCAAGGTGGGCATCCTGTGCATCAT | |
| | GAGCGACAGGGACCTGTACGACAAGCTGCAGTTCACCAGCCTG | |
| | GAGATCCCCAGGAGGAACGTGGCCACCCTGCAG | |
| NSP14 | GCCGAGAACGTGACCGGCCTGTTCAAGGACTGCAGCAAGGTGA | 357 |
| | TCACCGGCCTGCACCCCACCCAGGCCCCCACCCACCTGAGCGT | |
| | GGACACCAAGTTCAAGACCGAGGGCCTGTGCGTGGACATCCCC | |
| | GGCATCCCCAAGGACATGACCTACAGGAGGCTGATCAGCATGAT | |
| | GGGCTTCAAGATGAACTACCAGGTGAACGGCTACCCCAACATGT | |
| | TCATCACCAGGGAGGAGGCCATCAGGCACGTGAGGGCCTGGAT | |
| | CGGCTTCGACGTGGAGGGCTGCCACGCCACCAGGGAGGCCGT | |
| | GGGCACCAACCTGCCCCTGCAGCTGGGCTTCAGCACCGGCGTG | |
| | AACCTGGTGGCCGTGCCCACCGGCTACGTGGACACCCCCAACA | |
| | ACACCGACTTCAGCAGGGTGAGCGCCAAGCCCCCCCCCGGCGA | |
| | CCAGTTCAAGCACCTGATCCCCCTGATGTACAAGGGCCTGCCCT | |
| | GGAACGTGGTGAGGATCAAGATCGTGCAGATGCTGAGCGACAC | |
| | CCTGAAGAACCTGAGCGACAGGGTGGTGTTCGTGCTGTGGGCC | |
| | CACGGCTTCGAGCTGACCAGCATGAAGTACTTCGTGAAGATCGG | |
| | CCCCGAGAGGACCTGCTGCCTGTGCGACAGGAGGGCCACCTGC | |
| | TTCAGCACCGCCAGCGACACCTACGCCTGCTGGCACCACAGCAT | |
| | CGGCTTCGACTACGTGTACAACCCCTTCATGATCGACGTGCAGC | |
| | AGTGGGGCTTCACCGGCAACCTGCAGAGCAACCACGACCTGTA | |
| | CTGCCAGGTGCACGGCAACGCCCACGTGGCCAGCTGCGACGCC | |
| | ATCATGACCAGGTGCCTGGCCGTGCACGAGTGCTTCGTGAAGA | |
| | GGGTGGACTGGACCATCGAGTACCCCATCATCGGCGACGAGCT | |
| | GAAGATCAACGCCGCCTGCAGGAAGGTGCAGCACATGGTGGTG | |
| | AAGGCCGCCCTGCTGGCCGACAAGTTCCCCGTGCTGCACGACA | |
| | TCGGCAACCCCAAGGCCATCAAGTGCGTGCCCCAGGCCGACGT | |
| | GGAGTGGAAGTTCTACGACGCCCAGCCCTGCAGCGACAAGGCC | |
| | TACAAGATCGAGGAGCTGTTCTACAGCTACGCCACCCACAGCGA | |
| | CAAGTTCACCGACGGCGTGTGCCTGTTCTGGAACTGCAACGTGG | |
| | ACAGGTACCCCGCCAACAGCATCGTGTGCAGGTTCGACACCAG | |
| | GGTGCTGAGCAACCTGAACCTGCCCGGCTGCGACGGCGGCAGC | |
| | CTGTACGTGAACAAGCACGCCTTCCACACCCCCGCCTTCGACAA | |
| | GAGCGCCTTCGTGAACCTGAAGCAGCTGCCCTTCTTCTACTACA | |
| | GCGACAGCCCCTGCGAGAGCCACGGCAAGCAGGTGGTGAGCG | |
| | ACATCGACTACGTGCCCCTGAAGAGCGCCACCTGCATCACCAGG | |
| | TGCAACCTGGGCGGCGCCGTGTGCAGGCACCACGCCAACGAGT | |
| | ACAGGCTGTACCTGGACGCCTACAACATGATGATCAGCGCCGGC | |
| | TTCAGCCTGTGGGTGTACAAGCAGTTCGACACCTACAACCTGTG | |
| | GAACACCTTCACCAGGCTGCAG | |
| NSP15 | AGCCTGGAGAACGTGGCCTTCAACGTGGTGAACAAGGGCCACTT | 358 |
| | CGACGGCCAGCAGGGCGAGGTGCCCGTGAGCATCATCAACAAC | |
| | ACCGTGTACACCAAGGTGGACGGCGTGGACGTGGAGCTGTTCG | |
| | AGAACAAGACCACCCTGCCCGTGAACGTGGCCTTCGAGCTGTG | |
| | GGCCAAGAGGAACATCAAGCCCGTGCCCGAGGTGAAGATCCTG | |
| | AACAACCTGGGCGTGGACATCGCCGCCAACACCGTGATCTGGG | |
| | ACTACAAGAGGGACGCCCCCGCCCACATCAGCACCATCGGCGT | |
| | GTGCAGCATGACCGACATCGCCAAGAAGCCCACCGAGACCATCT | |
| | GCGCCCCCCTGACCGTGTTCTTCGACGGCAGGGTGGACGGCCA | |
| | GGTGGACCTGTTCAGGAACGCCAGGAACGGCGTGCTGATCACC | |
| | GAGGGCAGCGTGAAGGGCCTGCAGCCCAGCGTGGGCCCCAAG | |
| | CAGGCCAGCCTGAACGGCGTGACCCTGATCGGCGAGGCCGTGA | |
| | AGACCCAGTTCAACTACTACAAGAAGGTGGACGGCGTGGTGCAG | |
| | CAGCTGCCCGAGACCTACTTCACCCAGAGCAGGAACCTGCAGG | |
| | AGTTCAAGCCCAGGAGCCAGATGGAGATCGACTTCCTGGAGCTG | |
| | GCCATGGACGAGTTCATCGAGAGGTACAAGCTGGAGGGCTACG | |
| | CCTTCGAGCACATCGTGTACGGCGACTTCAGCCACAGCCAGCTG | |
| | GGCGGCCTGCACCTGCTGATCGGCCTGGCCAAGAGGTTCAAGG | |
| | AGAGCCCCTTCGAGCTGGAGGACTTCATCCCCATGGACAGCACC | |
| | GTGAAGAACTACTTCATCACCGACGCCCAGACCGGCAGCAGCAA | |
| | GTGCGTGTGCAGCGTGATCGACCTGCTGCTGGACGACTTCGTG | |
| | GAGATCATCAAGAGCCAGGACCTGAGCGTGGTGAGCAAGGTGG | |
| | TGAAGGTGACCATCGACTACACCGAGATCAGCTTCATGCTGTGG | |
| | TGCAAGGACGGCCACGTGGAGACCTTCTACCCCAAGCTGCAG | |
| NSP16 | AGCAGCCAGGCCTGGCAGCCCGGCGTGGCCATGCCCAACCTGT | 359 |
| | ACAAGATGCAGAGGATGCTGCTGGAGAAGTGCGACCTGCAGAA | |
| | CTACGGCGACAGCGCCACCCTGCCCAAGGGCATCATGATGAAC | |
| | GTGGCCAAGTACACCCAGCTGTGCCAGTACCTGAACACCCTGAC | |
| | CCTGGCCGTGCCCTACAACATGAGGGTGATCCACTTCGGCGCC | |
| | GGCAGCGACAAGGGCGTGGCCCCCGGCACCGCCGTGCTGAGG | |
| | CAGTGGCTGCCCACCGGCACCCTGCTGGTGGACAGCGACCTGA | |
| | ACGACTTCGTGAGCGACGCCGACAGCACCCTGATCGGCGACTG | |
| | CGCCACCGTGCACACCGCCAACAAGTGGGACCTGATCATCAGC | |
| | GACATGTACGACCCCAAGACCAAGAACGTGACCAAGGAGAACGA | |
| | CAGCAAGGAGGGCTTCTTCACCTACATCTGCGGCTTCATCCAGC | |
| | AGAAGCTGGCCCTGGGCGGCAGCGTGGCCATCAAGATCACCGA | |
| | GCACAGCTGGAACGCCGACCTGTACAAGCTGATGGGCCACTTC | |
| | GCCTGGTGGACCGCCTTCGTGACCAACGTGAACGCCAGCAGCA | |
| | GCGAGGCCTTCCTGATCGGCTGCAACTACCTGGGCAAGCCCAG | |
| | GGAGCAGATCGACGGCTACGTGATGCACGCCAACTACATCTTCT | |
| | GGAGGAACACCAACCCCATCCAGCTGAGCAGCTACAGCCTGTTC | |
| | GACATGAGCAAGTTCCCCCTGAAGCTGAGGGGCACCGCCGTGA | |
| | TGAGCCTGAAGGAGGGCCAGATCAACGACATGATCCTGAGCCT | |
| | GCTGAGCAAGGGCAGGCTGATCATCAGGGAGAACAACAGGGTG | |
| | GTGATCAGCAGCGACGTGCTGGTGAACAAC | |

The vaccine compositions described herein protects against disease caused by one or more coronavirus variants or coronavirus subvariants. In some embodiments, the coronavirus variants or coronavirus subvariants comprise past or Currently circulating coronavirus variants or coronavirus subvariants including but not limited to alpha, beta, gamma, delta, and omicron. In other embodiments, the coronavirus variants or coronavirus subvariants comprise future variants or future subvariants of human and animal coronavirus.

The vaccine compositions described herein may also protect against infection and reinfection of coronavirus variants or coronavirus subvariants. In some embodiments, the coronavirus variants or coronavirus subvariants comprise past or currently circulating coronavirus variants or coronavirus subvariants including but not limited to alpha, beta, gamma, delta, and omicron. In other embodiments, the coronavirus variants or coronavirus subvariants comprise future variants or future Subvariants of human and animal coronavirus.

The vaccine compositions described herein protects against infection or reinfection of one or more coronavirus variant or coronavirus subvariant. In some embodiments, the vaccine composition described herein against infection or reinfection of multiple coronavirus variants or coronavirus subvariants. In other embodiments, the vaccine composition described herein composition protects against infection or re-infection caused by one coronavirus variants or coronavirus subvariants.

In some embodiments, the vaccine composition induces strong and long-lasting protection mediated by antibodies (Abs), CD4+ T helper (Th1) cells, and/or CD8+ cytotoxic T-cells (CTL).

Molecular Adjuvants and T Cell Enhancements

In certain embodiments, the vaccine composition comprises a molecular adjuvant and/or one or more T Cell enhancement compositions. The adjuvant and/or enhancement compositions may help improve the immunogenicity and/or long-term memory of the vaccine composition. Non-limiting examples of molecular adjuvants include CpG, such as a CpG polymer, and flagellin,

In some embodiments, the vaccine composition comprises a T cell attracting chemokine. The T cell attracting chemokine helps pull the T cells from the circulation to the appropriate tissues, e.g., the lungs, heart, kidney, and brain. Non-limiting examples of T cell attracting chemokines include CCL5, CXCL9, CXCL10, CXCL11, CCL25, CCL28, CXCL14, CXCL17, or a combination thereof.

In some embodiments, the vaccine composition comprises a composition that promotes T cell proliferation. Non-limiting examples of compositions that promote T cell proliferation include IL-7, IL-15, IL-2, or a combination thereof.

In some embodiments, the vaccine composition comprises a composition that promotes T cell homing in the lungs. Non-limiting examples of compositions that promote T cell homing include CCL25, CCL28, CXCL14, CXCL17 or a combination thereof.

In certain embodiments, the molecular adjuvant and/or the T cell attracting chemokine and/or the composition that promotes T cell proliferation are delivered with a separate antigen delivery system from the large sequences.

Table 11 shows non-limiting examples of T-cell enhancements that may be used to create a vaccine composition described herein.

TABLE 11

| T-cell | | SEQ ID |
|---|---|---|
| enhancement | Sequence | NO: |
| CXCL11 | ATGAACAGGAAGGTGACCGCCATCGCCCTGGCCGCCATCATCTGG | 160 |
| | GCCACCGCCGCCCAGGGCTTCCTGATGTTCAAGCAGGGCAGGTGC | |
| | CTGTGCATCGGCCCCGGCATGAAGGCCGTGAAGATGGCCGAGATC | |
| | GAGAAGGCCAGCGTGATCTACCCCAGCAACGGCTGCGACAAGGTG | |
| | GAGGTGATCGTGACCATGAAGGCCCACAAGAGGCAGAGGTGCCTG | |
| | GACCCCAGGAGCAAGCAGGCCAGGCTGATCATGCAGGCCATCGAG | |
| | AAGAAGAACTTCCTGAGGAGGCAGAACATGTGA | |
| CCL5 | ATGAAGGTCTCCGCGGCAGCCCTCGCTGTCATCCTCATTGCTACTG | 161 |
| | CCCTCTGCGCTCCTGCATCTGCCTCCCCATATTCCTCGGACACCAC | |
| | ACCCTGCTGCTTTGCCTACATTGCCCGCCCACTGCCCCGTGCCCAC | |
| | ATCAAGGAGTATTTCTACACCAGTGGCAAGTGCTCCAACCCAGCAG | |
| | TCGTCCACAGGTCAAGGATGCCAAAGAGAGAGGGACAGCAAGTCTG | |
| | GCAGGATTTCCTGTATGACTCCCGGCTGAACAAGGGCAAGCTTTGT | |
| | CACCCGAAAGAACCGCCAAGTGTGTGCCAACCCAGAGAAGAAATGG | |
| | GTTCGGGAGTACATCAACTCTTTGGAGATGAGCTAGGATGGAGAGT | |
| | CCTTGAACCTGAACTTACACAAATTTGCCTGTTTCTGCTTGCTCTTGT | |
| | CCTAGCTTGGGAGGCTTCCCCTCACTATCCTACCCCACCCGCTCCT | |
| | TGA | |
| CXCL9 | ATGAAGAAAAGTGGTGTTCTTTTCCTCTTGGGCATCATCTTGCTGGT | 162 |
| | TCTGATTGGAGTGCAAGGAACCCCAGTAGTGAGAAAGGGTCGCTGT | |
| | TCCTGCATCAGCACCAACCAAGGGACTATCCACCTACAATCCTTGAA | |
| | AGACCTTAAACAATTTGCCCCAAGCCCTTCCTGCGAGAAAATTGAAA | |
| | TCATTGCTACACTGAAGAATGGAGTTCAAACATGTCTAAACCCAGAT | |
| | TCAGCAGATGTGAAGGAACTGATTAAAAAGTGGGAGAAACAGGTCA | |
| | GCCAAAAGAAAAAGCAAAAGAATGGGAAAAAACATGAAAAAAAGAAA | |
| | GTTCTGAAAGTTCGAAAATCTCAACGTTCTCGTCAAAAGAAGACTAC | |
| | ATAA | |
| CXCL10 | ATGAATCAAACTGCCATTCTGATTTGCTGCCTTATCTTTCTGACTCTA | 163 |
| | AGTGGCATTCAAGGAGTACCTCTCTCTAGAACTGTACGCTGTACCTG | |
| | CATCAGCATTAGTAATCAACCTGTTAATCCAAGGTCTTTAGAAAAACT | |
| | TGAAATTATTCCTGCAAGCCAATTTTGTCCACGTGTTGAGATCATTG | |
| | CTACAATGAAAAAGAAGGGTGAGAAGAGATGTCTGAATCCAGAATC | |
| | GAAGGCCATCAAGAATTTACTGAAAGCAGTTAGCAAGGAAAGGTCTA | |
| | AAAGATCTCCTTAA | |
| CXCL14 | ATGAGGCTCCTGGCGGCCGCGCTGCTCCTGCTGCTGCTGGCGCTG | 164 |
| | TACACCGCGCGTGTGGACGGGTCCAAATGCAAGTGCTCCCGGAAG | |
| | GGACCCAAGATCCGCTACAGCGACGTGAAGAAGCTGGAAATGAAGC | |
| | CAAAGTACCCGCACTGCGAGGAGAAGATGGTTATCATCACCACCAA | |
| | GAGCGTGTCCAGGTACCGAGGTCAGGAGCACTGCCTGCACCCCAA | |
| | GCTGCAGAGCACCAAGCGCTTCATCAAGTGGTACAACGCCTGGAAC | |
| | GAGAAGCGCAGGGTCTACGAAGAATAG | |
| CXCL17 | ATGAAAGTTCTAATCTCTTCCCTCCTCCTGTTGCTGCCACTAATGCT | 165 |
| | GATGTCCATGGTCTCTAGCAGCCTGAATCCAGGGGTCGCCAGAGGC | |
| | CACAGGGACCGAGGCCAGGCTTCTAGGAGATGGCTCCAGGAAGGC | |
| | GGCCAAGAATGTGAGTGCAAAGATTGGTTCCTGAGAGCCCCGAGAA | |
| | GAAAATTCATGACAGTGTCTGGGCTGCCAAAGAAGCAGTGCCCCTG | |
| | TGATCATTTCAAGGGCAATGTGAAGAAAACAAGACACCAAAGGCAC | |
| | CACAGAAAGCCAAACAAGCATTCCAGAGCCTGCCAGCAATTTCTCAA | |
| | ACAATGTCAGCTAAGAAGCTTTGCTCTGCCTTTGTAG | |
| CCL25 | ATGAACCTGTGGCTCCTGGCCTGCCTGGTGGCCGGCTTCCTGGGA | 166 |
| | GCCTGGGCCCCCGCTGTCCACACCCAAGGTGTCTTTGAGGACTGCT | |
| | GCCTGGCCTACCACTACCCCATTGGGTGGGCTGTGCTCCGGCGCG | |
| | CCTGGACTTACCGGATCCAGGAGGTGAGCGGGAGCTGCAATCTGC | |
| | CTGCTGCGATATTCTACCTCCCCAAGAGACACAGGAAGGTGTGTGG | |
| | GAACCCCAAAAGCAGGGAGGTGCAGAGAGCCATGAAGCTCCTGGA | |
| | TGCTCGAAATAAGGTTTTTGCAAAGCTCCACCACAACACGCAGACCT | |
| | TCCAAGCAGGCCCTCATGCTGTAAAGAAGTTGAGTTCTGGAAACTC | |
| | CAAGTTATCATCGTCCAAGTTTAGCAATCCCATCAGCAGCAGTAAGA | |
| | GGAATGTCTCCCTCCTGATATCAGCTAATTCAGGACTGTGA | |
| CCL28 | ATGCAGCAGAGAGGACTCGCCATCGTGGCCTTGGCTGTCTGTGCG | 167 |
| | GCCCTACATGCCTCAGAAGCCATACTTCCCATTGCCTCCAGCTGTTG | |
| | CACGGAGGTTTCACATCATATTTCCAGAAGGCTCCTGGAAAGAGTG | |
| | AATATGTGTCGCATCCAGAGAGCTGATGGGGATTGTGACTTGGCTG | |
| | CTGTCATCCTTCATGTCAAGCGCAGAAGAATCTGTGTCAGCCCGCA | |
| | CAACCATACTGTTAAGCAGTGGATGAAAGTGCAAGCTGCCAAGAAA | |
| | AATGGTAAAGGAAATGTTTGCCACAGGAAGAAACACCATGGCAAGA | |
| | GGAACAGTAACAGGGCACATCAGGGGAAACACGAAACATACGGCCA | |
| | TAAAACTCCTTATTAG | |
| IL-7 | ATGTTCCACGTGAGCTTCAGGTACATCTTCGGCATCCCCCCCCTGAT | 168 |
| | CCTGGTGCTGCTGCCCGTGACCAGCAGCGAGTGCCACATCAAGGA | |
| | CAAGGAGGGCAAGGCCTACGAGAGCGTGCTGATGATCAGCATCGA | |
| | CGAGCTGGACAAGATGACCGGCACCGACAGCAACTGCCCCAACAA | |
| | CGAGCCCAACTTCTTCAGGAAGCACGTGTGCGACGACACCAAGGAG | |
| | GCCGCCTTCCTGAACAGGGCCGCCAGGAAGCTGAAGCAGTTCCTG | |
| | AAGATGAACATCAGCGAGGAGTTCAACGTGCACCTGCTGACCGTGA | |
| | GCCAGGGCACCCAGACCCTGGTGAACTGCACCAGCAAGGAGGAGA | |
| | AGAACGTGAAGGAGCAGAAGAAGAACGACGCCTGCTTCCTGAAGAG | |
| | GCTGCTGAGGGAGATCAAGACCTGCTGGAACAAGATCCTGAAGGG | |
| | CAGCATCTGA | |
| IL-15 | ATGAGAATTTCGAAACCACATTTGAGAAGTATTTCCATCCAGTGCTA | 169 |
| | CTTGTGTTTACTTCTAAACAGTCATTTTCTAACTGAAGCTGGCATTCA | |
| | TGTCTTCATTTTGGGCTGTTTCAGTGCAGGGCTTCCTAAAACAGAAG | |
| | CCAACTGGGTGAATGTAATAAGTGATTTGAAAAAAATTGAAGATCTTA | |
| | TTCAATCTATGCATATTGATGCTACTTTATATACGGAAAGTGATGTTC | |
| | ACCCCAGTTGCAAAGTAACAGCAATGAAGTGCTTTCTCTTGGAGTTA | |
| | CAAGTTATTTCACTTGAGTCCGGAGATGCAAGTATTCATGATACAGT | |
| | AGAAAATCTGATCATCCTAGCAAACAACAGTTTGTCTTCTAATGGGA | |
| | ATGTAACAGAATCTGGATGCAAAGAATGTGAGGAACTGGAGGAAAA | |
| | AAATATTAAAGAATTTTTGCAGAGTTTTGTACATATTGTCCAAATGTT | |
| | CATCAACACTTCTTGA | |
| IL-2 | ATGTACAGGATGCAACTCCTGTCTTGCATTGCACTAAGTCTTGCACT | 170 |
| | TGTCACAAACAGTGCACGTACTTCAAGTTCTACAAAGAAAACACAGC | |
| | TACAACTGGAGCATTTACTGCTGGATTTACAGATGATTTTGAATGGA | |
| | ATTAATAATTACAAGAATCCCAAACTCACCAGGATGCTCACATTTAAG | |
| | TTTTACATGCCCAAGAAGGCCACAGAACTGAAACATCTTCAGTGTCT | |
| | AGAAGAAGAACTCAAACCTCTGGAGGAAGTGCTAAATTTAGCTCAAA | |
| | GCAAAAACTTTCACTTAAGACCCAGGGACTTAATCAGCAATATCAAC | |
| | GTAATAGTTCTGGAACTAAAGGGATCTGAAACAACATTCATGTGTGA | |
| | ATATGCTGATGAGACAGCAACCATTGTAGAATTTCTGAACAGATGGA | |
| | TTACCTTTGTCAAAGCATCATCTCAACACTGACTTGA | |

In preferred embodiments, the T-cell enhancement compositions described herein (e.g. CXCL9, CXCL10, IL-7, IL-2) may be integrated into a separate delivery system from the vaccine compositions. In some embodiments, the T-cell enhancement compositions described herein (e.g. CXCL9, CXCL1G, IL-7, IL-2) may be integrated into the same delivery system as the vaccine compositions.

In certain embodiments, the vaccine composition comprises a tag. For example, in some embodiments, the vaccine composition comprises a His tag. The present invention is not limited to a His tag and includes other tags such as those known to one of ordinary skill in the art, such as a fluorescent tag (e g. GFP, YFP, etc.), etc.

### Antigen Delivery System

The present invention also features vaccine compositions in the form of an antigen delivery system. Any appropriate antigen delivery system may be considered for delivery of the antigens described herein. The present invention is not limited to the antigen delivery systems described herein.

In certain embodiments, the antigen delivery system is for targeted delivery of the vaccine composition, e.g., for targeting to the tissues of the body where the virus replicates.

In certain embodiments, the antigen delivery system comprises adenoviruses such as but not limited to Ad5, Ad26, Ad35, etc., as well as carriers such as lipid nanoparticles, polymers, peptides, etc. In other embodiments, the antigen delivery system comprises a vesicular stomatitis virus (VSV) vector.

The present invention is not limited to adenovirus vector-based antigen delivery systems. In certain embodiments, the antigen delivery system comprises an adeno-associated virus vector-based antigen delivery system, such as but not limited to the adeno-associated virus vector type 9 (AAV9 serotype), AAV type 8 (AAV8 serotype), etc. In certain embodiments, the adeno-associated virus vectors used are tropic, e.g., tropic to lungs, brain, heart and kidney, e.g., the tissues of the body that express ACE2 receptors (FIG. 3A)). For example, AAV9 is known to be neurotropic, which would help the vaccine composition to be expressed in the brain.

In the antigen delivery system, the one or more large sequences are operatively linked to a promoter. In certain embodiments, the one or more large sequences are operatively linked to a generic promoter. For example, in certain embodiments, the one or more large sequences are operatively linked to a CMV promoter. In certain embodiments, the one or more large sequences are operatively linked to a CAG, EFIA, EFS, CBh, SFFV, MSCV, mPGK, hPGK, SV40, UBC, or another appropriate promoter.

In some embodiments, the one or more large sequences are operatively linked to a tissue-specific promoter (e.g., a lung-specific promoter). For example, the antigen may be operatively linked to a SpB promoter or a CD144 promoter.

As discussed, in certain embodiments, the vaccine composition comprises a molecular adjuvant. In certain embodiments, the molecular adjuvant is operatively linked to a generic promoter, e.g., as described above. In certain embodiments, the molecular adjuvant is operatively linked to a tissue-specific promoter, e.g., a lung-specific promoter, e.g., SpB or CD144.

As discussed, in certain embodiments, the vaccine composition comprises a T cell attracting chemokine. In certain embodiments, the T cell attracting chemokine is operatively linked to a generic promoter, e.g., as described above. In certain embodiments, the T cell attracting chemokine is operatively linked to a tissue-specific promoter; e.g.; a lung-specific promoter, e.g.; CpB or CD144,

As discussed, in certain embodiments, the vaccine composition comprises a composition for promoting T cell proliferation. In certain embodiments, the composition for promoting T cell proliferation is operatively linked to a generic promoter, e.g., as described above. In certain embodiments, the composition for promoting T cell proliferation is operatively linked to a tissue-specific promoter, e.g., a lung-specific promoter, e.g.; SpB or CD144.

Table 12 shows non-limiting examples of promoters that may be used to create a vaccine composition described herein.

TABLE 12

| | | SEQ ID |
|---|---|---|
| Promoter | Sequence | NO: |
| CAG | CTCGACATTGATTATTGACTAGTTATTAATAGTAATCAATTACGGGGTCAT | 171 |
| | TAGTTCATAGCCCATATATGGAGTTCCGCGTTACATAACTTACGGTAAAT | |
| | GGCCCGCCTGGCTGACCGCCCAACGACCCCCGCCCATTGACGTCAATA | |
| | ATGACGTATGTTCCCATAGTAACGCCAATAGGGACTTTCCATTGACGTCA | |
| | ATGGGTGGAGTATTTACGGTAAACTGCCCACTTGGCAGTACATCAAGTG | |
| | TATCATATGCCAAGTACGCCCCCTATTGACGTCAATGACGGTAAATGGC | |
| | CCGCCTGGCATTATGCCCAGTACATGACCTTATGGGACTTTCCTACTTG | |
| | GCAGTACATCTACGTATTAGTCATCGCTATTACCATGGTCGAGGTGAGC | |
| | CCCACGTTCTGCTTCACTCTCCCCATCTCCCCCCCCTCCCCACCCCCAA | |
| | TTTTGTATTTATTTATTTTTTAATTATTTTGTGCAGCGATGGGGGCGGGG | |
| | GGGGGGGGGGGGCGCGCGCCAGGCGGGGCGGGGCGGGGCGAGGGG | |
| | CGGGGCGGGGCGAGGCGGAGAGGTGCGGCGGCAGCCAATCAGAGCG | |
| | GCGCGCTCCGAAAGTTTCCTTTTATGGCGAGGCGGCGGCGGCGGCGG | |
| | CCCTATAAAAAGCGAAGCGCGCGGCGGGCGGGAGTCGCTGCGCGCTG | |
| | CCTTCGCCCCGTGCCCCGCTCCGCCGCCGCCTCGCGCCGCCCGCCCC | |
| | GGCTCTGACTGACCGCGTTACTCCCACAGGTGAGCGGGCGGGACGGC | |
| | CCTTCTCCTCCGGGCTGTAATTAGCGCTTGGTTTAATGACGGCTTGTTTC | |
| | TTTTCTGTGGCTGCGTGAAAGCCTTGAGGGGCTCCGGGAGGGCCCTTT | |
| | GTGCGGGGGGAGCGGCTCGGGGGGTGCGTGCGTGTGTGTGTGCGTGG | |
| | GGAGCGCCGCGTGCGGCTCCGCGCTGCCCGGCGGCTGTGAGCGCTGC | |
| | GGGCGCGGCGCGGGGCTTTGTGCGCTCCGCAGTGTGCGCGAGGGGAG | |
| | CGCGGCCGGGGGCGGTGCCCCGCGGTGCGGGGGGGGCTGCGAGGG | |
| | GAACAAAGGCTGCGTGCGGGGTGTGTGCGTGGGGGGGTGAGCAGGGG | |
| | GTGTGGGCGCGTCGGTCGGGCTGCAACCCCCCCTGCACCCCCCTCCC | |
| | CGAGTTGCTGAGCACGGCCCGGCTTCGGGTGCGGGGCTCCGTACGGG | |
| | GCGTGGCGCGGGGCTCGCCGTGCCGGGCGGGGGGTGGCGGCAGGTG | |
| | GGGGTGCCGGGCGGGGCGGGGCCGCCTCGGGCCGGGGAGGGCTCG | |
| | GGGGAGGGGCGCGGCGGCCCCCGGAGCGCCGGCGGCTGTCGAGGCG | |
| | CGGCGAGCCGCAGCCATTGCCTTTTATGGTAATCGTGCGAGAGGGCGC | |
| | AGGGACTTCCTTTGTCCCAAATCTGTGCGGAGCCGAAATCTGGGAGGC | |
| | GCCGCCGCACCCCCTCTAGCGGGCGCGGGGCGAAGCGGTGCGGCGC | |
| | CGGCAGGAAGGAAATGGGCGGGGAGGGCCTTCGTGCGTCGCCGCGCC | |
| | GCCGTCCCCTTCTCCCTCTCCAGCCTCGGGGCTGTCCGCGGGGGGAC | |
| | GGCTGCCTTCGGGGGGGACGGGGCAGGGCGGGGTTCGGCTTCTGGCG | |
| | TGTGACCGGCGGCTCTAGAGCCTCTGCTAACCATGTTCATGCCTTCTTC | |
| | TTTTTCCTACAGCTCCTGGGCAACGTGCTGGTTATTGTGCTGTCTCATCA | |
| | TTTGGCAAAGAATTG | |
| CMV | TAGTTATTAATAGTAATCAATTACGGGGTCATTAGTTCATAGCCCATATAT | 172 |
| | GGAGTTCCGCGTTACATAACTTACGGTAAATGGCCCGCCTGGCTGACCG | |
| | CCCAACGACCCCCGCCCATTGACGTCAATAATGACGTATGTTCCCATAG | |
| | TAACGCCAATAGGGACTTTCCATTGACGTCAATGGGTGGAGTATTTACG | |
| | GTAAACTGCCCACTTGGCAGTACATCAAGTGTATCATATGCCAAGTACG | |
| | CCCCCTATTGACGTCAATGACGGTAAATGGCCCGCCTGGCATTATGCCC | |
| | AGTACATGACCTTATGGGACTTTCCTACTTGGCAGTACATCTACGTATTA | |
| | GTCATCGCTATTACCATGGTGATGCGGTTTTGGCAGTACATCAATGGGC | |
| | GTGGATAGCGGTTTGACTCACGGGGATTTCCAAGTCTCCACCCCATTGA | |
| | CGTCAATGGGAGTTTGTTGGCACCAAAATCAACGGGACTTTCCAAAAT | |
| | GTCGTAACAACTCCGCCCCATTGACGCAAATGGGCGGTAGGCGTGTAC | |
| | GGTGGGAGGTCTATATAAGCAGAGCTGGTTTAGTGAACCGTCAGATC | |
| SP-B | GTATAGGGCTGTCTGGGAGCCACTCCAGGGCCACAGAAATCTTGTCTCT | 173 |
| | GACTCAGGGTATTTTGTTTTCTGTTTTGTGTAAATGCTCTTCTGACTAATG | |
| | CAAACCATGTGTCCATAGAACCAGAAGATTTTTCCAGGGGAAAAGGTAA | |
| | GGAGGTGGTGAGAGTGTCCTGGGTCTGCCCTTCCAGGGCTTGCCCTGG | |
| | GTTAAGAGCCAGGCAGGAAGCTCTCAAGAGCATTGCTCAAGAGTAGAG | |
| | GGGGCCTGGGAGGCCCAGGGAGGGGATGGGAGGGGAACACCCAGGC | |
| | TGCCCCCAACCAGATGCCCTCCACCCTCCTCAACCTCCCTCCCACGGC | |
| | CTGGAGAGGTGGGACCAGGTATGGAGGCTTGAGAGCCCCTGGTTGGAG | |
| | GAAGCCACAAGTCCAGGAACATGGGAGTCTGGGCAGGGGGCAAAGGA | |
| | GGCAGGAACAGGCCATCAGCCAGGACAGGTGGTAAGGCAGGCAGGAG | |
| | TGTTCCTGCTGGGAAAAGGTGGGATCAAGCACCTGGAGGGCTCTTCAG | |
| | AGCAAAGACAAACACTGAGGTCGCTGCCACTCCTACAGAGCCCCCACG | |
| | CCCCGCCCAGCTATAAGGGGCCATGCACCAAGCAGGGTACCCAGGCTG | |
| | CAGAGGTGCC | |
| CD144 | CATCCATGCCCATGGCCTCAGATGCCAGCCATAAGCTGTTGGGTTCCAA | 174 |
| | ACCTCGACTCCAGGCTGGACTCACCCCTGTCTCCCCCACCAGCCTGAC | |
| | ACCTCCACCTGGGTATCTAACGAGCATCTCAAACTCAACCTGCCTGAGA | |
| | CAGAGGAATCACTATCCCCTCCTCCTCCAAAAATATCCTTCCATCACACT | |
| | CCCCATCTTGTGCTCTGATTTACTAAACGGCCCTGGGCCCTCTCTTTCTC | |
| | AGGGTCTCTGCTTGCCCAGCTATATAATAAAACAAGTTTGGGACTTCCCA | |
| | ACCATTCACCCATGGAAAAACAGAAGCAACTCTTCAAAGGACAGATTCC | |
| | CAGGATCTGCCCTGGGAGATTCCAAATCAGTTGATCTGGGGTGAGCCCA | |
| | GTCCTCTGTAGTTTTTAGAAGCTCCTCCTATGTCTCTCCTGGTCAGCAGA | |
| | ATCTTGGCCCCTCCCTTCCCCCCAGCCTCTTGGTTCTTCTGGGCTCTGA | |
| | TCCAGCCTCAGCGTCACTGTCTTCCACGCCCCTCTTTGATTCTCGTTTAT | |
| | GTCAAAAGCCTTGTGAGGATGAGGCTGTGATTATCCCCATTTACAGAT | |
| | GAGGAAACTGTGGCTCCAGGATGACACAACTGGCCAGAGGTCACATCA | |
| | GAAGCAGAGCTGGGTCACTTGACTCCACCCAATATCCCTAAATGCAAAC | |
| | ATCCCCTACAGACCGAGGCTGGCACCTTAGAGCTGGAGTCCATGCCCG | |
| | CTCTGACCAGGAGAAGCCAACCTGGTCCTCCAGAGCCAAGAGCTTCTGT | |
| | CCCTTTCCCATCTCCTGAAGCCTCCCTGTCACCTTTAAAGTCCATTCCCA | |
| | CAAAGACATCATGGGATCACCACAGAAAATCAAGCTCTGGGGCTAGGCT | |
| | GACCCCAGCTAGATTTTTGGCTCTTTTATACCCCAGCTGGGTGGACAAG | |
| | CACCTTAAACCCGCTGAGCCTCAGCTTCCCGGGCTATAAAATGGGGGTG | |
| | ATGACACCTGCCTGTAGCATTCCAAGGAGGGTTAAATGTGATGCTGCAG | |
| | CCAAGGGTCCCCACAGCCAGGCTCTTTGCAGGTGCTGGGTTCAGAGTC | |
| | CCAGAGCTGAGGCCGGGAGTAGGGGTTCAAGTGGGGTGCCCCAGGCA | |
| | GGGTCCAGTGCCAGCCCTCTGTGGAGACAGCCATCCGGGGCCGAGGC | |
| | AGCCGCCCACCGCAGGGCCTGCCTATCTGCAGCCAGCCCAGCCCTCAC | |
| | AAAGGAACAATAACAGGAAACCATCCCAGGGGGAAGTGGGCCAGGGCC | |
| | AGCTGGAAAACCTGAAGGGGAGGCAGCCAGGCCTCCCTCGCCAGCGG | |
| | GGTGTGGCTCCCCTCCAAAGACGGTCGGCTGACAGGCTCCACAGAGCT | |
| | CCACTCACGCTCAGCCCTGGACGGACAGGCAGTCCAACGGAACAGAAA | |
| | CATCCCTCAGCCCACAGGCACGGTGAGTGGGGGCTCCCACACTCCCCT | |
| | CCACCCCAAACCCGCCACCCTGCGCCCAAGATGGGAGGGTCCTCAGCT | |
| | TCCCCATCTGTAGAATGGGCATCGTCCCACTCCCATGACAGAGAGGCTC | |
| | C | |
| wild type | ATGTTCGTGTTCCTGGTGCTGCTGCCCCTGGTGAGCAGC | 175 |
| native | | |
| leader | | |
| sequence | | |

In certain embodiments, the T cell attracting chemokine and the composition that promotes T cell proliferation are driven by the same promoter (e.g., the T cell attracting chemokine and the composition that promotes T cell proliferation are synthesized as a peptide). In certain embodiments, the T cell attracting chemokine and the composition that promotes T cell proliferation are driven by different promoters. In certain embodiments, the antigen, the T cell attracting chemokine, and the composition that promotes T cell proliferation are driven by the same promoter. In certain embodiments, the antigen, the T cell attracting chemokine, and the composition that promotes T cell proliferation are driven by the different promoters. In certain embodiments, the T cell attracting chemokine and the composition that promotes T cell proliferation are driven by the same promoter, and the one or more large sequences are driven by a different promoter.

In some embodiments, the antigen delivery system comprises one or more linkers between the T cell attracting chemokine and the composition that promotes T cell proliferation. In certain embodiments, linkers are used between one or more of the epitopes. The linkers may allow for cleavage of the separate molecules (e.g., chemokine). For example, in some embodiments, a linker is positioned between IL-7 (or IL-2) and CCL5, CXCL9, CXCL10, CXCL11, CCL25, CCL28, CXCL14, CXCL17, etc. In some embodiments, a linker is positioned between IL-15 and CCL5, CXCL9, CXCL10, CXCL11, CCL25, CCL28, CXCL14, CXCL17, etc. In some embodiments, a linker is positioned between the antigen or large sequence and another composition, e.g., IL-15. IL-7, CCL5, CXCL9, CXCL10. CXCL11, CCL25, CCL28, CXCL14, CXCL17, etc. A non-limiting example of a linker is T2A, E2A, P2A (see Table 13), or the like. The composition may feature a different linker between each open reading frame.

TABLE 13

| | | SEQ |
|---|---|---|
| | SEQUENCE | ID NO: |
| T2A Linker | GGAAGCGGAGAGGGCAGGGGAAGTCTT | 176 |
| | CTAACATGCGGGGACGTGGAGGAAAAT | |
| | CCCGGCCCC | |
| E2A Linker | GGAAGCGGACAGTGTACTAATTATGCT | 177 |
| | CTCTTGAAATTGGCTGGAGATGTTGAG | |
| | AGCAACCCAGGTCCC | |
| P2A Linker | GGAAGCGGAGCCACGAACTTCTCTCTG | 178 |
| | TTAAAGCAAGCAGGAGATGTTGAAGAA | |
| | AACCCCGGGCCT | |
| | GCCGCCTAC | 179 |
| | GGCCCCGGCCCCGGC | 180 |
| 6-His Tag | CATCACCATCACCATCAC | 181 |

The present invention includes mRNA sequences encoding any of the vaccine compositions or portions thereof herein, e.g., a molecular adjuvant, a T cell enhancement, etc. The present invention also includes modified mRNA sequences encoding any of the vaccine compositions or portions thereof herein. The present invention also includes DNA sequence encoding any of the vaccine compositions or portions thereof herein.

In certain embodiments, nucleic acids of a vaccine composition herein are chemically modified. In some embodiments, the nucleic acids of a vaccine composition therein are unmodified. In some embodiments, all or a portion of the uracil in the open reading frame has a chemical modification. In some embodiments, a chemical modification is in the 5-position of the uracil. In some embodiments, a chemical modification is a N1-methyl pseudouridine. In some embodiments, all or a portion of the uracil in the open reading frame has a N1-methyl pseudouridine in the 5-position of the uracil.

In certain embodiments, an open reading frame of a vaccine composition herein encodes one antigen or epitopes. In some embodiments, an open reading frame of a vaccine composition herein encodes two or more antigens or epitopes. In some embodiments, an open reading frame of a vaccine composition herein encodes five or more antigens or epitopes. In some embodiments, an open reading frame of a vaccine composition herein encodes ten or more antigens or epitopes. In some embodiments, an open reading frame of a vaccine composition herein encodes 50 or more antigens or epitopes.

### Methods

In some embodiments, the method comprises determining one or more conserved large sequences that are derived from coronavirus sequences (e.g., SARS-CoV-2, variants, common cold coronaviruses, previously known coronavirus strains, animal coronaviruses, etc.). The method may comprise selecting at least one large conserved sequence and synthesizing an antigen (or antigens) comprising the selected large conserved sequence(s). The method may comprise synthesizing a nucleotide composition (e.g., DNA, modified DNA. mRNA, modified mRNA, antigen delivery system, etc.) encoding the antigen comprising the selected large conserved sequence(s). In some embodiments, the method further comprises creating a vaccine composition comprising the antigen, nucleotide compositions, and/or antigen delivery system and a pharmaceutical carrier. In some embodiments, the large sequences comprise one or more conserved epitopes described herein, e.g., one or more conserved B-cell target epitopes and/or one or more conserved CD4+ T cell target epitopes and/or one or more conserved CD8+ T cell target epitopes.

In some embodiments, each of the large sequences are conserved among two or a combination of: at least two SARS-CoV-2 human strains in current circulation, at least one coronavirus that has caused a previous human outbreak, at least one coronavirus isolated from bats, at least one coronavirus isolated from pangolin, at least one coronavirus isolated from civet cats, at least one coronavirus strain isolated from mink, and at least one coronavirus strain isolated from camels or any other animal that is receptive to coronavirus.

As previously discussed, the compositions described herein, e.g., the antigens, the vaccine compositions, the antigen delivery systems, the chemokines, the adjuvants, etc. may be used to prevent a coronavirus disease in a subject. In some embodiments, the compositions described herein, e.g.; the antigens, the vaccine compositions, the antigen delivery systems, the chemokines, the adjuvants, etc. may be used to prevent a coronavirus infection prophylactically in a subject. In some embodiments, the compositions described herein, e.g., the antigens, the vaccine compositions, the antigen delivery systems, the chemokines, the adjuvants, etc. may elicit an immune response in a subject. In some embodiments, the compositions described herein, e.g., the antigens, the vaccine compositions, the antigen delivery systems, the chemokines, the adjuvants, etc. may prolong an immune response induced by the multi-epitope pan-coronavirus vaccine composition and increases T-cell migration to the lungs.

Methods for preventing a coronavirus disease in a subject may comprise administering to the subject a therapeutically effective amount of a pan-coronavirus vaccine composition according to the present invention. In some embodiments, the composition elicits an immune response in the subject. In some embodiments, the composition induces memory B and T cells. In some embodiments, the composition induces resident memory T cells (Trm), in some embodiments, the composition prevents virus replication, e.g., in the areas where the virus normally replicates such as lungs, brain, heart, and kidney. In some embodiments, the composition prevents a cytokine storm, e.g., in the areas where the virus normally replicates such as lungs, brain, heart, and kidney. In some embodiments, the composition prevents inflammation or an inflammatory response, e.g., in the areas where the virus normally replicates such as lungs, brain, heart, and kidney. In some embodiments, the composition improves homing and retention of T cells, e.g., in the areas where the virus normally replicates such as lungs, brain, heart, and kidney.

Methods for preventing a coronavirus infection prophylactically in a subject may comprise administering to the subject a prophylactically effective amount of a pan-coronavirus vaccine composition according to the present invention. In some embodiments, the composition elicits an immune response in the subject. In some embodiments, the composition induces memory B and T cells. In some embodiments, the composition induces resident memory T cells (Trm). In some embodiments, the composition prevents virus replication, e.g., in the areas where the virus normally replicates such as lungs, brain, heart, and kidney. In some embodiments, the composition prevents a cytokine storm, e.g., in the areas where the virus normally replicates such as lungs, brain, heart, and kidney. In some embodiments, the composition prevents inflammation or an inflammatory response, e.g., in the areas where the virus normally replicates such as lungs, brain, heart, and kidney. In some embodiments, the composition improves homing and retention of T cells, e.g., in the areas where the virus normally replicates such as lungs, brain, heart, and kidney.

Methods for eliciting an immune response in a subject may comprise administering to the subject a vaccine composition according to the present invention, wherein the composition elicits an immune response in the subject. In some embodiments, the composition induces memory B and T cells. In some embodiments, the composition induces resident memory T cells (Trm). In some embodiments, the composition prevents virus replication, e.g., in the areas where the virus normally replicates such as lungs, brain, heart, and kidney. In some embodiments, the composition prevents a cytokine storm, e.g., in the areas where the virus normally replicates such as lungs, brain, heart, and kidney. In some embodiments, the composition prevents inflammation or an inflammatory response, e.g., in the areas where the virus normally replicates such as lungs, brain, heart, and kidney. In some embodiments, the composition improves homing and retention of T cells, e.g., in the areas where the virus normally replicates such as lungs, brain, heart, and kidney.

Methods for prolonging an immune response induced by a vaccine composition of the present invention and increasing T cell migration to particular tissues (e.g., lung, brain, heart, kidney, etc.) may comprise co-expressing a T-cell attracting chemokine, a composition that promotes T cell proliferation, and a vaccine composition (e.g., antigen) according to the present invention.

Methods for prolonging the retention of memory T-cell into the lungs induced by a vaccine composition of the present invention and increasing virus-specific tissue resident memory T-cells (TRM cells) may comprise co-expressing a T-cell attracting chemokine, a composition that promotes T cell proliferation, and a vaccine composition (e.g., antigen) according to the present invention.

The vaccine composition may be administered through standard means, e.g., through an intravenous route (i.v.), an intranasal route (i.n.), or a sublingual route (s.l.) route.

In certain embodiments, the method comprises administering to the subject a second (e.g., booster) dose. The second dose may comprise the same vaccine composition or a different vaccine composition. Additional doses of one or more vaccine compositions may be administered.

### Sequential Vaccine Delivery Methodology

In some embodiments, the present invention features a method of delivering the vaccine to induce heterologous immunity in a subject (e.g., prime/boost, see FIG. 25B and FIG. 26B). In some embodiments, the method comprises administering a first pan-coronavirus vaccine composition dose using a first delivery system. In further embodiments, the method comprises administering a second vaccine composition dose using a second delivery system. In some embodiments, the second composition is administered 8 days after administration of the first composition. In some embodiments, the second composition is administered 9 days after administration of the first composition. In some embodiments, the second composition is administered 10 days after administration of the first composition. In some embodiments, the second composition is administered 11 days after administration of the first composition. In some embodiments, the second composition is administered 12 days after administration of the first composition. In some embodiments, the second composition is administered 13 days after administration of the first composition. In some embodiments, the second composition is administered 14 days after administration of the first composition. In some embodiments, the second composition is administered from 14 to 30 days after administration of the first composition. In some embodiments, the second composition is administered from 30 to 60 days after administration of the first composition. In other embodiments, the first delivery system and the second delivery system are different. In some embodiments, the peptide vaccine composition is administered 14-days after the administration of the first vaccine composition dose. In some embodiments, the peptide vaccine composition is administered 30 or 60 days after the administration of the first vaccine composition dose.

In some embodiments, the first delivery system or the second delivery system comprises an mRNA, a modified mRNA or a peptide vector. In other embodiments, the peptide vector comprises adenovirus or an adeno-associated virus vector.

In some embodiments, the present invention features a method of delivering the vaccine to induce heterologous immunity in a subject (i.e. prime/pull, see FIG. 25A and FIG. 26A). In some embodiments, the method comprises administering a pan-coronavirus vaccine composition. In further embodiments, the method comprises administering at least one T-cell attracting chemokine after administering the pan-coronavirus vaccine composition. In some embodiments, the T-cell attracting chemokine is administered 8 days after the vaccine composition is administered. In some embodiments, the T-cell attracting chemokine is administered 9 days after the vaccine composition is administered. In some embodiments, the T-cell attracting chemokine is administered 10 days after the vaccine composition is administered. In some embodiments, the T-cell attracting chemokine is administered 11 days after the vaccine composition is administered. In some embodiments, the T-cell attracting chemokine is administered 12 days after the vaccine composition is administered. In some embodiments, the T-cell attracting chemokine is administered 13 days after the vaccine composition is administered. In some embodiments, the T-cell attracting chemokine is administered 14 days after the vaccine composition is administered. In some embodiments, the T-cell attracting chemokine is administered from 14 to 30 days after administration of the vaccine composition. In some embodiments, the T-cell attracting chemokine is administered from 30 to 60 days after administration of the vaccine composition. In some embodiments, the T cell-attracting chemokine composition is administered 8 to 14-days after the administration of the final vaccine composition dose. In some embodiments, the cell-attracting chemokine composition is administered 30 or 60 days after the administration of the final vaccine composition dose.

The present invention also features a novel “prime, pull, and boost” strategy. In other embodiments, the present invention features a method to increase the size and maintenance of lung-resident B-cells, CD4+ T cells and CD8+ T cells to protect against SARS-CoV-2 (FIG. 25D and FIG. 26D). In some embodiments, the method comprises administering a pan-coronavirus vaccine composition. In other embodiments, the method comprises administering at least one T-cell attracting chemokine after administering the pan-coronavirus vaccine composition. In further embodiments, the method comprises administering at least one cytokine after administering the T-cell attracting chemokine. In some embodiments, the T-cell attracting chemokine is administered 14 days after administering the pan-coronavirus composition. In other embodiments, the cytokine is administered 10 days after administering the T-cell attracting chemokine. In some embodiments, the T-cell attracting chemokine is administered 8 days after the vaccine composition is administered. In some embodiments, the T-cell attracting chemokine is administered 9 days after the vaccine composition is administered. In some embodiments, the T-cell attracting chemokine is administered 10 days after the vaccine composition is administered. In some embodiments, the T-cell attracting chemokine is administered 11 days after the vaccine composition is administered. In some embodiments, the T-cell attracting chemokine is administered 12 days after the vaccine composition is administered. In some embodiments, the T-cell attracting chemokine is administered 13 days after the vaccine composition is administered. In some embodiments, the T-cell attracting chemokine is administered 14 days after the vaccine composition is administered. In some embodiments, the T-cell attracting chemokine is administered from 14 to 30 days after administration of the vaccine composition. In some embodiments, the T-cell attracting chemokine is administered from 30 to 60 days after administration of the vaccine composition. In some embodiments, the cytokine is administered 8 days after administering the T-cell attracting chemokine. In some embodiments, the cytokine is administered 9 days after administering the T-cell attracting chemokine. In some embodiments, the cytokine is administered 10 days after administering the T-cell attracting chemokine. In some embodiments, the cytokine is administered 11 days after administering the T-cell attracting chemokine. In some embodiments, the cytokine is administered 12 days after administering the T-cell attracting chemokine. In some embodiments, the cytokine is administered 13 days after administering the T-cell attracting chemokine. In some embodiments, the cytokine is administered 14 days after administering the T-cell attracting chemokine. In some embodiments, the cytokine is administered from 14 to 30 days after administering the T-cell attracting chemokine. In some embodiments, the cytokine is administered from 30 to 60 days after administering the T-cell attracting chemokine. In some embodiments, the cytokine composition is administered 8 to 14-days after the administration of the T cell-attracting chemokine. In some embodiments, the cytokine composition is administered 30 or 60 days after the administration of the T cell-attracting chemokine.

The present invention further features a novel “prime, pull, and keep” strategy (FIG. 25C and FIG. 26C). In further embodiments, the present invention features a method to increase the size and maintenance of lung-resident B-cells, CD4+ T cells and CD8+ T cells to protect against SARS-CoV-2. In some embodiments, the method comprises administering a pan-coronavirus vaccine composition. In other embodiments, the method comprises administering at least one T-cell attracting chemokine after administering the pan-coronavirus vaccine composition. In further embodiments, the method comprises administering at least one mucosal chemokine after administering the T-cell attracting chemokine. In some embodiments, the T-cell attracting chemokine is administered 14 days after administering the pan-coronavirus composition. In other embodiments, the mucosal chemokines is administered 10 days after administering the T-cell attracting chemokine. In some embodiments, the T-cell attracting chemokine is administered 8 days after the vaccine composition is administered. In some embodiments, the T-cell attracting chemokine is administered 9 days after the vaccine composition is administered. In some embodiments, the T-cell attracting chemokine is administered 10 days after the vaccine composition is administered. In some embodiments, the T-cell attracting chemokine is administered 11 days after the vaccine composition is administered. In some embodiments, the T-cell attracting chemokine is administered 12 days after the vaccine composition is administered. In some embodiments, the T-cell attracting chemokine is administered 13 days after the vaccine composition is administered. In some embodiments, the T-cell attracting chemokine is administered 14 days after the vaccine composition is administered. In some embodiments, the T-cell attracting chemokine is administered from 14 to 30 days after administration of the vaccine composition. In some embodiments, the T-cell attracting chemokine is administered from 30 to 60 days after administration of the vaccine composition. In some embodiments, the mucosal chemokine is administered 8 days after administering the T-cell attracting chemokine. In some embodiments, the mucosal chemokine is administered 9 days after administering the T-cell attracting chemokine. In some embodiments, the mucosal chemokine is administered 10 days after administering the T-cell attracting chemokine. In some embodiments, the mucosal chemokine is administered 11 days after administering the T-cell attracting chemokine. In some embodiments, the mucosal chemokine is administered 12 days after administering the T-cell attracting chemokine. In some embodiments, the mucosal chemokine is administered 13 days after administering the T-cell attracting chemokine. In some embodiments, the mucosal chemokine is administered 14 days after administering the T-cell attracting chemokine. In some embodiments, the mucosal chemokine is administered from 14 to 30 days after administering the T-cell attracting chemokine. In some embodiments, the mucosal chemokine is administered from 30 to 60 days after administering the T-cell attracting chemokine. In some embodiments, the mucosal chemokine composition is administered 8 to 14-days after the administration of the T cell-attracting chemokine. In some embodiments, the mucosal cytokine composition is administered 30 or 60 days after the administration of the T cell-attracting chemokine.

In some embodiments, the mucosal chemokines may comprise CCL25, CCL28,CXCL14, CXCL17, or a combination thereof. In some embodiments, the T-cell attracting chemokines may comprise CCL5, CXCL9, CXCL10, CXC11, or a combination thereof. In some embodiments, the cytokines may comprise IL-15, IL-2, IL-7 or a combination thereof.

In some embodiments, the efficacy (or effectiveness) of a vaccine composition herein is greater than 60%. In some embodiments, the efficacy (or effectiveness) of a vaccine composition herein is greater than 70%. In some embodiments, the efficacy (or effectiveness) of a vaccine composition herein is greater than 80%. In some embodiments, the efficacy (or effectiveness) of a vaccine composition herein is greater than 90%. In some embodiments, the efficacy (or effectiveness) of a vaccine composition herein is greater than 95%.

Vaccine efficacy may be assessed using standard analyses (see, e.g., Weinberg et al., J Infect Dis. 2010 Jun. 1; 201(11):1607-10). For example, vaccine efficacy may be measured by double-blind, randomized, clinical controlled trials. Vaccine efficacy may be expressed as a proportionate reduction in disease attack rate (AR) between the unvaccinated (ARU) and vaccinated (ARV) study cohorts and can be calculated from the relative risk (RR) of disease among the vaccinated group with use of the following formulas: Efficacy=(ARU−ARV)/ARU×100; and Efficacy=(1−RR)×100.

Likewise, vaccine effectiveness may be assessed using standard analyses (see, e.g., Weinberg et al., J Infect Dis. 2010 Jun. 1; 201(11):1607-10). Vaccine effectiveness is an assessment of how a vaccine (which may have already proven to have high vaccine efficacy) reduces disease in a population. This measure can assess the net balance of benefits and adverse effects of a vaccination program, not just the vaccine itself, under natural field conditions rather than in a controlled clinical trial. Vaccine effectiveness is proportional to vaccine efficacy (potency) but is also affected by how well target groups in the population are immunized, as well as by other non-vaccine-related factors that influence the ‘real-world’ outcomes of hospitalizations, ambulatory visits, or costs. For example, a retrospective case control analysis may be used, in which the rates of vaccination among a set of infected cases and appropriate controls are compared. Vaccine effectiveness may be expressed as a rate difference, with use of the odds ratio (OR) for developing infection despite vaccination: Effectiveness=(1−OR)×100.

In some embodiments, the vaccine immunizes the subject against a coronavirus for up to 1 year. In some embodiments, the vaccine immunizes the subject against a coronavirus for up to 2 years. In some embodiments, the vaccine immunizes the subject against a coronavirus for more than 1 year, more than 2 years, more than 3 years, more than 4 years, or for 5-10 years.

In some embodiments, the subject is a young adult between the ages of about 20 years and about 50 years (e.g., about 20, 25, 30, 35, 40, 45 or 50 years old).

In some embodiments, the subject is an elderly subject about 60 years old, about 70 years old, or older (e.g., about 60, 65, 70, 75, 80, 85 or 90 years old).

In some embodiments, the subject is about 5 years old or younger. For example, the subject may be between the ages of about 1 year and about 5 years (e.g., about 1, 2, 3, 5 or 5 years), or between the ages of about 6 months and about 1 year (e.g., about 6, 7, 8, 9, 10, 11 or 12 months). In some embodiments, the subject is about 12 months or younger (e.g., 12, 11, 10, 9, 8, 7, 6, 5, 4, 3, 2 months or 1 month). In some embodiments, the subject is about 6 months or younger.

In some embodiments, the subject was born full term (e.g., about 37-42 weeks). In some embodiments, the subject was born prematurely, for example, at about 36 weeks of gestation or earlier (e.g., about 36, 35, 34, 33, 32, 31, 30, 29, 28, 27, 26 or 25 weeks). For example, the subject may have been born at about 32 weeks of gestation or earlier. In some embodiments, the subject was born prematurely between about 32 weeks and about 36 weeks of gestation. In such subjects, a vaccine may be administered later in life, for example, at the age of about 6 months to about 5 years, or older.

In some embodiments, the subject is pregnant (e.g., in the first, second or third trimester) when administered a vaccine.

In some embodiments, the subject has a chronic pulmonary disease (e.g., chronic obstructive pulmonary disease (COPD) or asthma) or is at risk thereof. Two forms of COPD include chronic bronchitis, which involves a long-term cough with mucus, and emphysema, which involves damage to the lungs over time. Thus, a subject administered a vaccine may have chronic bronchitis or emphysema.

In some embodiments, the subject has been exposed to a coronavirus. In some embodiments, the subject is infected with a coronavirus. In some embodiments, the subject is at risk of infection by a coronavirus.

In some embodiments, the subject is immunocompromised (has an impaired immune system, e.g., has an immune disorder or autoimmune disorder).

### Pharmaceutical Carriers

In certain embodiments, the vaccine composition further comprises a pharmaceutical carrier. Pharmaceutical carriers are well known to one of ordinary skill in the art. For example, in certain embodiments, the pharmaceutical carrier is selected from the group consisting of water, an alcohol, a natural or hardened oil, a natural or hardened wax, a calcium carbonate, a sodium carbonate, a calcium phosphate, kaolin, talc, lactose and combinations thereof. In some embodiments, the pharmaceutical carrier may comprise a lipid nanoparticle, an adenovirus vector, or an adeno-associated virus vector. In some embodiments, the vaccine composition is constructed using an adeno-associated virus vectors-based antigen delivery system.

Also provided herein is vaccine of any one of the foregoing paragraphs, formulated in a nanoparticle (e.g., a lipid nanoparticle). In some embodiments, the nanoparticle has a mean diameter of 50-200 nm. In some embodiments, the nanoparticle is a lipid nanoparticle. In some embodiments, the lipid nanoparticle comprises a cationic lipid, a PEG-modified lipid, a sterol and a non-cationic lipid. In some embodiments, the lipid nanoparticle comprises a molar ratio of about 20-60% cationic lipid, 0.5-15% PEG-modified lipid, 25-55% sterol, and 25% non-cationic lipid. In some embodiments, the cationic lipid is an ionizable cationic lipid, and the non-cationic lipid is a neutral lipid, and the sterol is a cholesterol. In some embodiments, the cationic lipid is selected from 2,2-dilinoleyl-4-dimethylaminoethyl-[1,3]-dioxolane (DLin-KC2-DMA), dilinoleyl-methyl-4-dimethylaminobutyrate (DLin-MC3-DMA), and di((Z)-non-2-en-1-yl) 9-((4-(dimethylamino)butanoyl)oxy)heptadecanedioate (L319).

### Hybrid Vaccine Candidates

Referring now to FIGS. 30, 31, 32A, and 32B, the present invention may further feature a pan-coronavirus-influenza recombinant vaccine composition. The composition comprises at least a portion of a coronavirus spike (S) protein and at least a portion of an influenza hemagglutinin (HA) protein.

In some embodiments, the portion of an influenza hemagglutinin (HA) protein is highly conserved among human influenza viruses. The portion of an influenza hemagglutinin (HA) protein may be derived from one or more of: H1N1 virus strain, H3N2 virus strain, influenza B virus strains, or variants thereof.

In some embodiments, the H1N1 virus strains or variants are selected from 28566 available complete genome sequences in NCBI for hemagglutinin (HA) gene. Some of the prominent strains are: OK384178.1, OM642156.1, OM654386.1, OL840606.1, OK625377.1, OM865246.1, OM935941.1, OM642158.1, OM935953.1, MW840068.1, MW839847.1, MW839825.1, MW930730.1, MT227010.1, LC638096.1, LC638077.1, LC637999.1, and LC645067.1. In some embodiments, the H3N2 virus strains or variants are selected from 33620 available complete genome sequences in NCBI for hemagglutinin (HA) gene. Some of the prominent strains are: MZ005227.1, MW849238.1, MZ203409.1, MZ198318.1, MZ198312.1, MZ198295.1, MZ198289.1, MZ198265.1, MW789449.1, MW798370.1, MW790182.1, MW789645.1, MW789778.1, MW789685.1, MW789659.1, and MW790001.1. In some embodiments, the influenza B virus strains or variants are selected from 16596 available complete genome sequences in NCBI for hemagglutinin (HA) gene. Some of the prominent strains are: M10298.1, MT7385253, MT808048.1, MT056751.1, MT314641.1, MT874090.1, MT242979.1, MT315665.1, MT105564.0.1, MT057563.1, MT056955.1, MT243019.1, MT306916.1, MT057571.1, MT314793.1, MT343026.1, MT874109.1, MT243795.1, MT315769.1, and KX885875.1.

Table 14: Shows non-limiting examples of a portion of an influenza hemagglutinin (HA) protein that may be used in accordance with the present invention.

| | | SEQ ID |
|---|---|---|
| | Sequence | NO: |
| HA (nucleotide) | TTCGGAGCTATTGCTGGTTTCTTGGAAGGAGGATGGGAAGGAATGA | 384 |
| | TTGCAGGTTGGCACGGATACACATCTCATGGAGCACATGGAGTAGC | |
| | AGTGGCAGCAGACCTTAAGAGTACCCA | |
| HA | FGAIAGFLEGGWEGMIAGWHGYTSHGAHGVAVAADLKSTX | 385 |
| (amino acid) | | |
| HA-H1N1 | ATGAAGGCAATACTAGTAGTTCTGCTATATACATTTGCAACCGCAAA | 386 |
| | TGCAGACACATTATGTATAGGTTATCATGCGAACAACTCAACAGACA | |
| | CTGTAGACACAGTACTAGAAAAGAATGTAACAGTAACACACTCTGTT | |
| | AACCTTCTAGAAGACAAGCATAACGGGAAACTATGCAAACTAAGAG | |
| | GGGTAGCCCCATTGCATTTGGGTAAATGTAACATTGCTGGCTGGAT | |
| | CCTGGGAAATCCAGAGTGTGAATCACTCTCCACAGCAAGCTCATGG | |
| | TCCTACATTGTGGAAACATCTAGTTCAGACAATGGAACGTGTTACCC | |
| | AGGAGATTTCATCGATTATGAGGAGCTAAGAGAGCAATTGAGCTCA | |
| | GTGTCATCATTTGAAAGGTTTGAGATATTCCCCAAGACAAGTTCATG | |
| | GCCCAATCATGACTCGAACAAAGGTGTAACGACAGCATGTCCTCAT | |
| | GCTGGAGCAAAAAGCTTCTACAAAAATTTAATATGGCTAGTTAAAAA | |
| | AGGAAATTCATACCCAAAGCTCAGCAAATCCTACATTAATGATAAAG | |
| | GGAAAGAAGTCCTCGTGCTATGGGGCATTCACCATCCACCTACTAG | |
| | TGCTGACCAACAAAGTCTCTATCAGAATGCAGATGCATATGTTTTTG | |
| | TGGGGACATCAAGATACAGCAAGAAGTTCAAGCCGGAAATAGCAAT | |
| | AAGGCCCAAAGTGAGGGATCAAGAAGGGAGAATGAACTATTACTGG | |
| | ACACTAGTAGAGCCGGGAGACAAAATAACATTCGAAGCAACTGGAA | |
| | ATCTAGTGGTACCGAGATATGCATTCGCAATGGAAAGAAATGCTGG | |
| | ATCTGGTATTATCATTTCAGATACACCAGTCCACGATTGCAATACAA | |
| | CTTGTCAGACACCCAAGGGTGCTATAAACACCAGCCTCCCATTTCA | |
| | GAACATACATCCGATCACAATTGGAAAATGTCCAAAATATGTAAAAA | |
| | GCACAAAATTGAGACTGGCCACAGGATTGAGGAATGTCCCGTCCAT | |
| | TCAATCTAGAGGCCTATTTGGGGCCATTGCCGGTTTCATTGAAGGG | |
| | GGGTGGACAGGGATGGTAGATGGATGGTACGGTTATCACCATCAAA | |
| | ATGAGCAGGGGTCAGGATATGCAGCCGACCTGAAGAGCACACAGA | |
| | ATGCCATTGACGAGATTACTAACAAAGTAAACTCTGTTATTGAAAAAA | |
| | TGAATACACAGTTCACAGCAGTAGGTAAAGAGTTCAACCACCTGGAA | |
| | AAAAGAATAGAGAATTTAAATAAAAAAGTTGATGATGGTTTCCTGGA | |
| | CATTTGGACTTACAATGCCGAACTGTTGGTTCTATTGGAAAATGAAA | |
| | GAACTTTGGACTACCACGATTCAAAGGTGAAGAACTTATATGAAAAG | |
| | GTAAGAAGCCAGTTAAAAAACAATGCCAAGGAAATTGGAAACGGCT | |
| | GCTTTGAATTTTACCACAAATGTGATAACACGTGCATGGAAAGTGTC | |
| | AAAAATGGGACTTATGACTACCCAAAATACTCAGAGGAAGCAAAATT | |
| | AAACAGAGAAGAAATAGATGGGGTAAAGCTGGAATCAACAAGGATT | |
| | TACCAGATTTTGGCGATCTATTCAACCGTCGCCAGTTCATTGGTACT | |
| | GGTAGTCTCCCTGGGGGCAATCAGTTTCTGGATGTGCTCTAATGGG | |
| | TCTCTACAGTGTAGAATATGTATTTAA | |
| HA H3N2 | AGCAAAAGCAGGGGATAATTCTATTAACCATGAAGACTATCATTGCT | 387 |
| | TTGAGCTACATTCTATGTCTGGTTTTCGCTCAAAAACTTCCTGGAAAT | |
| | GACAATAGCACTGCAACGCTGTGCCTTGGGCACCATGCAGTACCAA | |
| | ACGGAACGATAGTGAAAACAATCACGAATGACCAAATTGAAGTTACT | |
| | AATGCTACTGAGCTGGTTCAGAATTCCTCAATAGGTGAAATATGCGA | |
| | CAGTCCTCATCAGATCCTTGATGGAGAAAACTGCACACTAATAGATG | |
| | CTCTATTGGGAGACCCTCAGTGTGATGGCTTTCAAAATAAGAAATGG | |
| | GACCTTTTTGTTGAACGAAGCAAAGCCTACAGCAACTGTTACCCTTA | |
| | TGATGTGCCGGATTATGCCTCCCTTAGGTCACTAGTTGCCTCATCCG | |
| | GCACACTGGAGTTTAACAATGAAAGCTTCAATTGGGCTGGAGTCACT | |
| | CAAAACGGAACAAGTTCTGCTTGCATAAGGGGATCTAATAGTAGTTT | |
| | CTTTAGTAGATTAAATTGGTTGACCCACTTAAACTTCAAGTACCCAG | |
| | CATTGAACGTGACTATGCCAAACAATGAACAATTTGACAAATTGTAC | |
| | ATTTGGGGGGTTCACCACCCGGGTACGGACAAGGACCAAATCTTCC | |
| | TGTATGCTCAATCATCAGGAAGAATCACAGTATCTACCAAAAGAAGC | |
| | CAACAAGCTGTAATCCCGAATATCGGATCTAGACCCAGAATAAGGAA | |
| | TATCCCTAGCAGAATAAGCATCTATTGGACAATAGTAAAACCGGGAG | |
| | ACATACTTTTGATTAACAGCACAGGGAATCTAATTGCTCCTAGGGGT | |
| | TACTTCAAAATACGAAGTGGGAAAAGCTCAATAATGAGATCAGATGC | |
| | ACCCATTGGCAAATGCAAGTCTGAATGCATCACTCCAAATGGAAGCA | |
| | TTCCCAATGACAAACCATTCCAAAATGTAAACAGGATCACATACGGG | |
| | GCCTGTCCCAGATATGTTAAGCAAAGCACTCTGAAATTGGCAACAG | |
| | GAATGCGAAATGTACCAGAGAAACAAACTAGAGGCATATTTGGCGC | |
| | AATAGCGGGTTTCATAGAAAATGGTTGGGAGGGAATGGTGGATGGT | |
| | TGGTACGGTTTCAGGCATCAAAATTCTGAGGGAAGAGGACAAGCAG | |
| | CAGATCTCAAAAGCACTCAAGCAGCAATCGATCAAATCAATGGGAA | |
| | GCTGAATCGATTGATCGGGAAAACCAACGAGAAATTCCATCAGATTG | |
| | AGAAAGAATTCTCAGAAGTAGAAGGGAGAATTCAGGACCTTGAGAA | |
| | ATATGTTGAGGACACAAAAATAGATCTCTGGTCATACAACGCAGAGC | |
| | TTCTTGTTGCCCTGGAAAACCAACATACAATTGATCTAACTGACTCA | |
| | GAAATGAACAAACTGTTTGAAAAAACAAAGAAGCAACTGAGGGAAAA | |
| | TGCTGAGGATATGGGCAATGGTTGTTTCAAAATATACCACAAATGTG | |
| | ACAATGCCTGCATAGGATCAATCAGAAATGGAACTTATGACCACGAT | |
| | GTATACAGGGATGAAGCATTAAACAACCGGTTCCAGATCAAGGGAG | |
| | TTGAGCTGAAGTCAGGGTACAAAGATTGGATCCTATGGATTTCCTTT | |
| | GCCATATCATGTTTTTTGCTTTGTGTTGCTTTGTTGGGGTTCATCATG | |
| | TGGGCCTGCCAAAAGGGCAACATTAGGTGCAACATTTGCATTTGAG | |
| | TGCATTAATTAAAAACACCCTTGTTTCTACT | |
| HA influenza B | ATTTTCTAATATCCACAAAATGAAGGCAATAATTGTACTACTCATGGT | 388 |
| | AGTAACATCCAATGCAGATCGAATCTGCACTGGGATAACATCGTCAA | |
| | ACTCACCACATGTCGTCAAAACTGCTACTCAAGGGGAGGTCAACGT | |
| | GACCGGTGTAATACCACTGACAACAACACCCACCAAATCTCATTTTG | |
| | CAAATCTCAAAGGAACAGAAACCAGGGGGAAACTATGCCCAAAATG | |
| | CCTCAACTGCACAGATCTGGATGTAGCCTTGGGCAGACCAAAATGC | |
| | ACAGGGAAAATACCCTCTGCAAGGGTTTCAATACTCCATGAAGTCAG | |
| | ACCTGTTACATCTGGGTGCTTTCCTATAATGCACGATAGAACAAAAA | |
| | TTAGACAGCTGCCTAACCTTCTCCGAGGATACGAACATGTCAGGTTA | |
| | TCAACTCACAACGTTATCAATGCAAAAGATGCACCAGGAAGACCCTA | |
| | CGAAATTGGAACCTCAGGGTCTTGCCCTAACATTACCAATGGAAAC | |
| | GGATTCTTCGCAACAATGGCTTGGGCCGTCCCAAAAAACAAAACAG | |
| | CAACAAATCCATTAACAATAGAAGTACCATACATTTGTACAGAAGGA | |
| | GAAGACCAAATTACCGTTTGGGGGTTCCACTCTGACAACGAGACCC | |
| | AAATGGCAAAGCTCTATGGGGACTCAAAGCCCCAGAAGTTCACCTC | |
| | ATCTGCCAACGGAGTGACCACACATTACGTTTCACAGATTGGTGGC | |
| | TTCCCAAATCAAACAGAAGACGGAGGACTACCACAAAGTGGCAGAA | |
| | TTGTTGTTGATTACATGGTGCAGAAATCTGGAAAAACAGGAACAATT | |
| | ACCTATCAAAGAGGTATTTTATTGCCTCAAAAAGTGTGGTGCGCAAG | |
| | TGGCAGGAGCAAGGTAATAAAAGGATCCTTGCCCTTAATTGGAGAA | |
| | GCAGATTGCCTCCATGAAAAATACGGTGGATTAAACAAAAGCAAGC | |
| | CTTACTACACAGGGGAACATGCAAAGGCCATAGGAAATTGCCCAAT | |
| | ATGGGTGAAAACACCCTTGAAGCTGGCCAATGGAACCAAATATAGA | |
| | CCCCCTGCAAAACTATTAAAGGAAAGAGGTTTCTTCGGAGCCATTGC | |
| | TGGTTTCTTAGAGGGAGGATGGGAAGGAATGATTGCAGGTTGGCAC | |
| | GGATACACATCCCATGGGGCACATGGAGTAGCGGTGGCAGCTGAC | |
| | CTTAAGAGCACTCAAGAGGCCATAAACAAGATAACAAAAAATCTCAA | |
| | CTCTTTGAGTGAGCTGGAAGTAAAGAATCTTCAAAGACTAAGCGGTG | |
| | CCATGGATGAACTCCACAACGAAATACTAGAACTAGATGAGAAAGTG | |
| | GATGATCTCAGAGCTGACACAATAAGCTCACAAATAGAACTCGCAGT | |
| | CCTGCTTTCCAATGAAGGAATAATAAACAGTGAAGATGAACATCTCT | |
| | TGGCGCTTGAAAGAAAGCTGAAGAAAATGCTGGGCCCCTCTGCTGT | |
| | AGAGATAGGGAATGGATGCTTTGAAACCAAACACAAGTGCAACCAG | |
| | ACCTGCCTCGACAGAATAGCTGCTGGTACCTTTGATGCAGGAGAAT | |
| | TTTCTCTCCCCACCTTTGATTCACTGAATATTACTGCTGCATCTTTAA | |
| | ATGACGACGGATTGGACAATCATACTATACTGCTTTACTACTCAACT | |
| | GCTGCCTCCAGTTTGGCTGTAACACTGATGATAGCTATCTTTGTTGT | |
| | TTATATGGTCTCCAGAGACAATGTTTCTTGCTCCATTTGTCTATAAGG | |
| | AAAGTTAAGCCCTGTATTTTCCTTTATTGTAGTGCTTGTTTGCTTGTT | |
| | GTCATTACAAAGAAACGTTATTGAAAAAT | |

In some embodiments, the portion of a coronavirus spike (S) protein and the portion of an Influenza hemagglutinin (HA) protein are operatively linked to a generic promoter. The generic promoter may be a CMV or a AG promoter.

In some embodiments, the portion of a coronavirus spike (S) protein and the portion of an influenza hemagglutinin (HA) protein are separated by a linker. The linker may be 2 to 10 amino acids in length.

In some embodiments, the compositions described herein may be used to prevent a coronavirus and/or influenza infection prophylactically in a subject. In some embodiments, the compositions described herein may elicit an immune response in a subject.

TABLE 5
and FIG. 31 and 32B shows examples of vaccine compositions described herein.
The present invention is not limited to the examples in Table 4:

| Vaccine | | SEQ ID |
|---|---|---|
| Candidate | Sequence | NO: |
| 1 | TAGTTATTAATAGTAATCAATTACGGGGTCATTAGTTCATAGCCCATAT | 383 |
| promoter, | ATGGAGTTCCGCGTTACATAACTTACGGTAAATGGCCCGCCTGGCTG | |
| 5’UTR and | ACCGCCCAACGACCCCCGCCCATTGACGTCAATAATGACGTATGTTC | |
| leader | CCATAGTAACGCCAATAGGGACTTTCCATTGACGTCAATGGGTGGAG | |
| sequence. | TATTTACGGTAAACTGCCCACTTGGCAGTACATCAAGTGTATCATATG | |
| Spike | CCAAGTACGCCCCCTATTGACGTCAATGACGGTAAATGGCCCGCCTG | |
| glycoprotein | GCATTATGCCCAGTACATGACCTTATGGGACTTTCCTACTTGGCAGTA | |
| (HexaPro- | CATCTACGTATTAGTCATCGCTATTACCATGGTGATGCGGTTTTGGCA | |
| mutations), | GTACATCAATGGGCGTGGATAGCGGTTTGACTCACGGGGATTTCCAA | |
| 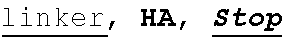 | GTCTCCACCCCATTGACGTCAATGGGAGTTTGTTTTGGCACCAAAAT | |
| 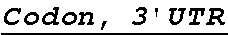 | CAACGGGACTTTCCAAAATGTCGTAACAACTCCGCCCCATTGACGCA | |
| 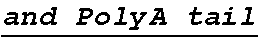 | AATGGGCGGTAGGCGTGTACGGTGGGAGGTCTATATAAGCAGAGCT | |
| | GGTTTAGTGAACCGTCAGATCGAGAATAAACTAGTATTCTTCTGGTCC | |
| | CCACAGACTCAGAGAGAACCCGCCACCATGTTCGTGTTCCTGGTGCT | |
| | GCTGCCCCTGGTGAGCAGCCAGTGCGTGAACCTGACCACCAGGACC | |
| | CAGCTGCCCCCCGCCTACACCAACAGCTTCACCAGGGGCGTGTACT | |
| | ACCCCGACAAGGTGTTCAGGAGCAGCGTGCTGCACAGCACCCAGGA | |
| | CCTGTTCCTGCCCTTCTTCAGCAACGTGACCTGGTTCCACGCCATCC | |
| | ACGTGAGCGGCACCAACGGCACCAAGAGGTTCGACAACCCCGTGCT | |
| | GCCCTTCAACGACGGCGTGTACTTCGCCAGCACCGAGAAGAGCAAC | |
| | ATCATCAGGGGCTGGATCTTCGGCACCACCCTGGACAGCAAGACCC | |
| | AGAGCCTGCTGATCGTGAACAACGCCACCAACGTGGTGATCAAGGT | |
| | GTGCGAGTTCCAGTTCTGCAACGACCCCTTCCTGGGCGTGTACTACC | |
| | ACAAGAACAACAAGAGCTGGATGGAGAGCGAGTTCAGGGTGTACAG | |
| | CAGCGCCAACAACTGCACCTTCGAGTACGTGAGCCAGCCCTTCCTG | |
| | ATGGACCTGGAGGGCAAGCAGGGCAACTTCAAGAACCTGAGGGAGT | |
| | TCGTGTTCAAGAACATCGACGGCTACTTCAAGATCTACAGCAAGCAC | |
| | ACCCCCATCAACCTGGTGAGGGACCTGCCCCAGGGCTTCAGCGCCC | |
| | TGGAGCCCCTGGTGGACCTGCCCATCGGCATCAACATCACCAGGTT | |
| | CCAGACCCTGCTGGCCCTGCACAGGAGCTACCTGACCCCCGGCGAC | |
| | AGCAGCAGCGGCTGGACCGCCGGCGCCGCCGCCTACTACGTGGGC | |
| | TACCTGCAGCCCAGGACCTTCCTGCTGAAGTACAACGAGAACGGCA | |
| | CCATCACCGACGCCGTGGACTGCGCCCTGGACCCCCTGAGCGAGAC | |
| | CAAGTGCACCCTGAAGAGCTTCACCGTGGAGAAGGGCATCTACCAG | |
| | ACCAGCAACTTCAGGGTGCAGCCCACCGAGAGCATCGTGAGGTTCC | |
| | CCAACATCACCAACCTGTGCCCCTTCGGCGAGGTGTTCAACGCCAC | |
| | CAGGTTCGCCAGCGTGTACGCCTGGAACAGGAAGAGGATCAGCAAC | |
| | TGCGTGGCCGACTACAGCGTGCTGTACAACAGCGCCAGCTTCAGCA | |
| | CCTTCAAGTGCTACGGCGTGAGCCCCACCAAGCTGAACGACCTGTG | |
| | CTTCACCAACGTGTACGCCGACAGCTTCGTGATCAGGGGCGACGAG | |
| | GTGAGGCAGATCGCCCCCGGCCAGACCGGCAAGATCGCCGACTACA | |
| | ACTACAAGCTGCCCGACGACTTCACCGGCTGCGTGATCGCCTGGAA | |
| | CAGCAACAACCTGGACAGCAAGGTGGGCGGCAACTACAACTACCTG | |
| | TACAGGCTGTTCAGGAAGAGCAACCTGAAGCCCTTCGAGAGGGACA | |
| | TCAGCACCGAGATCTACCAGGCCGGCAGCACCCCCTGCAACGGCGT | |
| | GGAGGGCTTCAACTGCTACTTCCCCCTGCAGAGCTACGGCTTCCAG | |
| | CCCACCAACGGCGTGGGCTACCAGCCCTACAGGGTGGTGGTGCTGA | |
| | GCTTCGAGCTGCTGCACGCCCCCGCCACCGTGTGCGGCCCCAAGAA | |
| | GAGCACCAACCTGGTGAAGAACAAGTGCGTGAACTTCAACTTCAACG | |
| | GCCTGACCGGCACCGGCGTGCTGACCGAGAGCAACAAGAAGTTCCT | |
| | GCCCTTCCAGCAGTTCGGCAGGGACATCGCCGACACCACCGACGCC | |
| | GTGAGGGACCCCCAGACCCTGGAGATCCTGGACATCACCCCCTGCA | |
| | GCTTCGGCGGCGTGAGCGTGATCACCCCCGGCACCAACACCAGCAA | |
| | CCAGGTGGCCGTGCTGTACCAGGACGTGAACTGCACCGAGGTGCCC | |
| | GTGGCCATCCACGCCGACCAGCTGACCCCCACCTGGAGGGTGTACA | |
| | GCACCGGCAGCAACGTGTTCCAGACCAGGGCCGGCTGCCTGATCG | |
| | GCGCCGAGCACGTGAACAACAGCTACGAGTGCGACATCCCCATCGG | |
| | CGCCGGCATCTGCGCCAGCTACCAGACCCAGACCAACAGCCCCGGC | |
| | AGCGCCAGCAGCGTGGCCAGCCAGAGCATCATCGCCTACACCATGA | |
| | GCCTGGGCGCCGAGAACAGCGTGGCCTACAGCAACAACAGCATCGC | |
| | CATCCCCACCAACTTCACCATCAGCGTGACCACCGAGATCCTGCCCG | |
| | TGAGCATGACCAAGACCAGCGTGGACTGCACCATGTACATCTGCGG | |
| | CGACAGCACCGAGTGCAGCAACCTGCTGCTGCAGTACGGCAGCTTC | |
| | TGCACCCAGCTGAACAGGGCCCTGACCGGCATCGCCGTGGAGCAG | |
| | GACAAGAACACCCAGGAGGTGTTCGCCCAGGTGAAGCAGATCTACA | |
| | AGACCCCCCCCATCAAGGACTTCGGCGGCTTCAACTTCAGCCAGATC | |
| | CTGCCCGACCCCAGCAAGCCCAGCAAGAGGAGCCCCATCGAGGAC | |
| | CTGCTGTTCAACAAGGTGACCCTGGCCGACGCCGGCTTCATCAAGC | |
| | AGTACGGCGACTGCCTGGGCGACATCGCCGCCAGGGACCTGATCTG | |
| | CGCCCAGAAGTTCAACGGCCTGACCGTGCTGCCCCCCCTGCTGACC | |
| | GACGAGATGATCGCCCAGTACACCAGCGCCCTGCTGGCCGGCACCA | |
| | TCACCAGCGGCTGGACCTTCGGCGCCGGCCCCGCCCTGCAGATCC | |
| | CCTTCCCCATGCAGATGGCCTACAGGTTCAACGGCATCGGCGTGAC | |
| | CCAGAACGTGCTGTACGAGAACCAGAAGCTGATCGCCAACCAGTTCA | |
| | ACAGCGCCATCGGCAAGATCCAGGACAGCCTGAGCAGCACCCCCAG | |
| | CGCCCTGGGCAAGCTGCAGGACGTGGTGAACCAGAACGCCCAGGC | |
| | CCTGAACACCCTGGTGAAGCAGCTGAGCAGCAACTTCGGCGCCATC | |
| | AGCAGCGTGCTGAACGACATCCTGAGCAGGCTGGACCCCCCCGAGG | |
| | CCGAGGTGCAGATCGACAGGCTGATCACCGGCAGGCTGCAGAGCCT | |
| | GCAGACCTACGTGACCCAGCAGCTGATCAGGGCCGCCGAGATCAGG | |
| | GCCAGCGCCAACCTGGCCGCCACCAAGATGAGCGAGTGCGTGCTG | |
| | GGCCAGAGCAAGAGGGTGGACTTCTGCGGCAAGGGCTACCACCTGA | |
| | TGAGCTTCCCCCAGAGCGCCCCCCACGGCGTGGTGTTCCTGCACGT | |
| | GACCTACGTGCCCGCCCAGGAGAAGAACTTCACCACCGCCCCCGCC | |
| | ATCTGCCACGACGGCAAGGCCCACTTCCCCAGGGAGGGCGTGTTCG | |
| | TGAGCAACGGCACCCACTGGTTCGTGACCCAGAGGAACTTCTACGA | |
| | GCCCCAGATCATCACCACCGACAACACCTTCGTGAGCGGCAACTGC | |
| | GACGTGGTGATCGGCATCGTGAACAACACCGTGTACGACCCCCTGC | |
| | AGCCCGAGCTGGACAGCTTCAAGGAGGAGCTGGACAAGTACTTCAA | |
| | GAACCACACCAGCCCCGACGTGGACCTGGGCGACATCAGCGGCATC | |
| | AACGCCAGCGTGGTGAACATCCAGAAGGAGATCGACAGGCTGAACG | |
| | AGGTGGCCAAGAACCTGAACGAGAGCCTGATCGACCTGCAGGAGCT | |
| | GGGCAAGTACGAGCAGTACATCAAGTGGCCCTGGTACATCTGGCTG | |
| | GGCTTCATCGCCGGCCTGATCGCCATCGTGATGGTGACCATCATGCT | |
| | GTGCTGCATGACCAGCTGCTGCAGCTGCCTGAAGGGCTGCTGCAGC | |
| | TGCGGCAGCTGCTGCAAGTTCGACGAGGACGACAGCGAGCCCGTG | |
| | CTGAAGGGCGTGAAGCTGCACTACACCGGAAGCGGAGCCACGAACT | |
| | TCTCTCTGTTAAAGCAAGCAGGAGATGTTGAAGAAAACCCCGGGCCT | |
| | 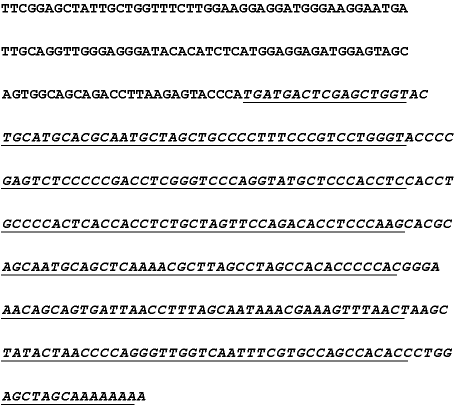 | |
| 2 | TAGTTATTAATAGTAATCAATTACGGGGTCATTAGTTCATAGCCCATAT | 390 |
| promoter, | ATGGAGTTCCGCGTTACATAACTTACGGTAAATGGCCCGCCTGGCTG | |
| 5’UTR and | ACCGCCCAACGACCCCCGCCCATTGACGTCAATAATGACGTATGTTC | |
| leader | CCATAGTAACGCCAATAGGGACTTTCCATTGACGTCAATGGGTGGAG | |
| sequence. | TATTTACGGTAAACTGCCCACTTGGCAGTACATCAAGTGTATCATATG | |
| Spike | CCAAGTACGCCCCCTATTGACGTCAATGACGGTAAATGGCCCGCCTG | |
| glycoprotein | GCATTATGCCCAGTACATGACCTTATGGGACTTTCCTACTTGGCAGTA | |
| (two proline | CATCTACGTATTAGTCATCGCTATTACCATGGTGATGCGGTTTTGGCA | |
| substitutions), | GTACATCAATGGGCGTGGATAGCGGTTTGACTCACGGGGATTTCCAA | |
| 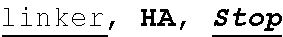 | GTCTCCACCCCATTGACGTCAATGGGAGTTTGTTTTGGCACCAAAAT | |
| 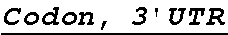 | CAACGGGACTTTCCAAAATGTCGTAACAACTCCGCCCCATTGACGCA | |
| 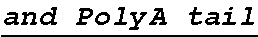 | AATGGGCGGTAGGCGTGTACGGTGGGAGGTCTATATAAGCAGAGCT | |
| | GGTTTAGTGAACCGTCAGATCGAGAATAAACTAGTATTCTTCcTGGTC | |
| | CCCACAGACTCAGAGAGAACCCGCCACCATGTTCGTGTTCCTGGTG | |
| | CTGCTGCCCCTGGTGAGCAGCCAGTGCGTGAACCTGACCACCAGGA | |
| | CCCAGCTGCCCCCCGCCTACACCAACAGCTTCACCAGGGGCGTGTA | |
| | CTACCCCGACAAGGTGTTCAGGAGCAGCGTGCTGCACAGCACCCAG | |
| | GACCTGTTCCTGCCCTTCTTCAGCAACGTGACCTGGTTCCACGCCAT | |
| | CCACGTGAGCGGCACCAACGGCACCAAGAGGTTCGACAACCCCGTG | |
| | CTGCCCTTCAACGACGGCGTGTACTTCGCCAGCACCGAGAAGAGCA | |
| | ACATCATCAGGGGCTGGATCTTCGGCACCACCCTGGACAGCAAGAC | |
| | CCAGAGCCTGCTGATCGTGAACAACGCCACCAACGTGGTGATCAAG | |
| | GTGTGCGAGTTCCAGTTCTGCAACGACCCCTTCCTGGGCGTGTACTA | |
| | CCACAAGAACAACAAGAGCTGGATGGAGAGCGAGTTCAGGGTGTAC | |
| | AGCAGCGCCAACAACTGCACCTTCGAGTACGTGAGCCAGCCCTTCC | |
| | TGATGGACCTGGAGGGCAAGCAGGGCAACTTCAAGAACCTGAGGGA | |
| | GTTCGTGTTCAAGAACATCGACGGCTACTTCAAGATCTACAGCAAGC | |
| | ACACCCCCATCAACCTGGTGAGGGACCTGCCCCAGGGCTTCAGCGC | |
| | CCTGGAGCCCCTGGTGGACCTGCCCATCGGCATCAACATCACCAGG | |
| | TTCCAGACCCTGCTGGCCCTGCACAGGAGCTACCTGACCCCCGGCG | |
| | ACAGCAGCAGCGGCTGGACCGCCGGCGCCGCCGCCTACTACGTGG | |
| | GCTACCTGCAGCCCAGGACCTTCCTGCTGAAGTACAACGAGAACGG | |
| | CACCATCACCGACGCCGTGGACTGCGCCCTGGACCCCCTGAGCGAG | |
| | ACCAAGTGCACCCTGAAGAGCTTCACCGTGGAGAAGGGCATCTACC | |
| | AGACCAGCAACTTCAGGGTGCAGCCCACCGAGAGCATCGTGAGGTT | |
| | CCCCAACATCACCAACCTGTGCCCCTTCGGCGAGGTGTTCAACGCC | |
| | ACCAGGTTCGCCAGCGTGTACGCCTGGAACAGGAAGAGGATCAGCA | |
| | ACTGCGTGGCCGACTACAGCGTGCTGTACAACAGCGCCAGCTTCAG | |
| | CACCTTCAAGTGCTACGGCGTGAGCCCCACCAAGCTGAACGACCTG | |
| | TGCTTCACCAACGTGTACGCCGACAGCTTCGTGATCAGGGGCGACG | |
| | AGGTGAGGCAGATCGCCCCCGGCCAGACCGGCAAGATCGCCGACT | |
| | ACAACTACAAGCTGCCCGACGACTTCACCGGCTGCGTGATCGCCTG | |
| | GAACAGCAACAACCTGGACAGCAAGGTGGGCGGCAACTACAACTAC | |
| | CTGTACAGGCTGTTCAGGAAGAGCAACCTGAAGCCCTTCGAGAGGG | |
| | ACATCAGCACCGAGATCTACCAGGCCGGCAGCACCCCCTGCAACGG | |
| | CGTGGAGGGCTTCAACTGCTACTTCCCCCTGCAGAGCTACGGCTTC | |
| | CAGCCCACCAACGGCGTGGGCTACCAGCCCTACAGGGTGGTGGTG | |
| | CTGAGCTTCGAGCTGCTGCACGCCCCCGCCACCGTGTGCGGCCCCA | |
| | AGAAGAGCACCAACCTGGTGAAGAACAAGTGCGTGAACTTCAACTTC | |
| | AACGGCCTGACCGGCACCGGCGTGCTGACCGAGAGCAACAAGAAGT | |
| | TCCTGCCCTTCCAGCAGTTCGGCAGGGACATCGCCGACACCACCGA | |
| | CGCCGTGAGGGACCCCCAGACCCTGGAGATCCTGGACATCACCCCC | |
| | TGCAGCTTCGGCGGCGTGAGCGTGATCACCCCCGGCACCAACACCA | |
| | GCAACCAGGTGGCCGTGCTGTACCAGGACGTGAACTGCACCGAGGT | |
| | GCCCGTGGCCATCCACGCCGACCAGCTGACCCCCACCTGGAGGGT | |
| | GTACAGCACCGGCAGCAACGTGTTCCAGACCAGGGCCGGCTGCCTG | |
| | ATCGGCGCCGAGCACGTGAACAACAGCTACGAGTGCGACATCCCCA | |
| | TCGGCGCCGGCATCTGCGCCAGCTACCAGACCCAGACCAACAGCCC | |
| | CAGGAGGGCCAGGAGCGTGGCCAGCCAGAGCATCATCGCCTACAC | |
| | CATGAGCCTGGGCGCCGAGAACAGCGTGGCCTACAGCAACAACAGC | |
| | ATCGCCATCCCCACCAACTTCACCATCAGCGTGACCACCGAGATCCT | |
| | GCCCGTGAGCATGACCAAGACCAGCGTGGACTGCACCATGTACATC | |
| | TGCGGCGACAGCACCGAGTGCAGCAACCTGCTGCTGCAGTACGGCA | |
| | GCTTCTGCACCCAGCTGAACAGGGCCCTGACCGGCATCGCCGTGGA | |
| | GCAGGACAAGAACACCCAGGAGGTGTTCGCCCAGGTGAAGCAGATC | |
| | TACAAGACCCCCCCCATCAAGGACTTCGGCGGCTTCAACTTCAGCCA | |
| | GATCCTGCCCGACCCCAGCAAGCCCAGCAAGAGGAGCTTCATCGAG | |
| | GACCTGCTGTTCAACAAGGTGACCCTGGCCGACGCCGGCTTCATCA | |
| | AGCAGTACGGCGACTGCCTGGGCGACATCGCCGCCAGGGACCTGAT | |
| | CTGCGCCCAGAAGTTCAACGGCCTGACCGTGCTGCCCCCCCTGCTG | |
| | ACCGACGAGATGATCGCCCAGTACACCAGCGCCCTGCTGGCCGGCA | |
| | CCATCACCAGCGGCTGGACCTTCGGCGCCGGCGCCGCCCTGCAGA | |
| | TCCCCTTCGCCATGCAGATGGCCTACAGGTTCAACGGCATCGGCGT | |
| | GACCCAGAACGTGCTGTACGAGAACCAGAAGCTGATCGCCAACCAG | |
| | TTCAACAGCGCCATCGGCAAGATCCAGGACAGCCTGAGCAGCACCG | |
| | CCAGCGCCCTGGGCAAGCTGCAGGACGTGGTGAACCAGAACGCCC | |
| | AGGCCCTGAACACCCTGGTGAAGCAGCTGAGCAGCAACTTCGGCGC | |
| | CATCAGCAGCGTGCTGAACGACATCCTGAGCAGGCTGGACCCCCCC | |
| | GAGGCCGAGGTGCAGATCGACAGGCTGATCACCGGCAGGCTGCAG | |
| | AGCCTGCAGACCTACGTGACCCAGCAGCTGATCAGGGCCGCCGAGA | |
| | TCAGGGCCAGCGCCAACCTGGCCGCCACCAAGATGAGCGAGTGCGT | |
| | GCTGGGCCAGAGCAAGAGGGTGGACTTCTGCGGCAAGGGCTACCA | |
| | CCTGATGAGCTTCCCCCAGAGCGCCCCCCACGGCGTGGTGTTCCTG | |
| | CACGTGACCTACGTGCCCGCCCAGGAGAAGAACTTCACCACCGCCC | |
| | CCGCCATCTGCCACGACGGCAAGGCCCACTTCCCCAGGGAGGGCG | |
| | TGTTCGTGAGCAACGGCACCCACTGGTTCGTGACCCAGAGGAACTT | |
| | CTACGAGCCCCAGATCATCACCACCGACAACACCTTCGTGAGCGGC | |
| | AACTGCGACGTGGTGATCGGCATCGTGAACAACACCGTGTACGACC | |
| | CCCTGCAGCCCGAGCTGGACAGCTTCAAGGAGGAGCTGGACAAGTA | |
| | CTTCAAGAACCACACCAGCCCCGACGTGGACCTGGGCGACATCAGC | |
| | GGCATCAACGCCAGCGTGGTGAACATCCAGAAGGAGATCGACAGGC | |
| | TGAACGAGGTGGCCAAGAACCTGAACGAGAGCCTGATCGACCTGCA | |
| | GGAGCTGGGCAAGTACGAGCAGTACATCAAGTGGCCCTGGTACATC | |
| | TGGCTGGGCTTCATCGCCGGCCTGATCGCCATCGTGATGGTGACCA | |
| | TCATGCTGTGCTGCATGACCAGCTGCTGCAGCTGCCTGAAGGGCTG | |
| | CTGCAGCTGCGGCAGCTGCTGCAAGTTCGACGAGGACGACAGCGAG | |
| | CCCGTGCTGAAGGGCGTGAAGCTGCACTACACCGGAAGCGGAGCCA | |
| | CGAACTTCTCTCTGTTAAAGCAAGCAGGAGATGTTGAAGAAAACCCC | |
| | 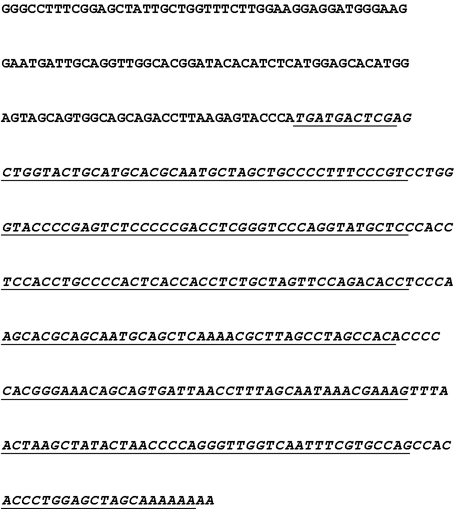 | |
| 3 | CTCGACATTGATTATTGACTAGTTATTAATAGTAATCAATTACGGGGT | 391 |
| promoter, | CATTAGTTCATAGCCCATATATGGAGTTCCGCGTTACATAACTTACGG | |
| 5’UTR and | TAAATGGCCCGCCTGGCTGACCGCCCAACGACCCCCGCCCATTGAC | |
| leader | GTCAATAATGACGTATGTTCCCATAGTAACGCCAATAGGGACTTTCCA | |
| sequence. | TTGACGTCAATGGGTGGAGTATTTACGGTAAACTGCCCACTTGGCAG | |
| Spike | TACATCAAGTGTATCATATGCCAAGTACGCCCCCTATTGACGTCAATG | |
| glycoprotein | ACGGTAAATGGCCCGCCTGGCATTATGCCCAGTACATGACCTTATGG | |
| (HexaPro- | GACTTTCCTACTTGGCAGTACATCTACGTATTAGTCATCGCTATTACC | |
| mutations), | ATGGTCGAGGTGAGCCCCACGTTCTGCTTCACTCTCCCCATCTCCCC | |
| 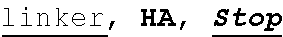 | CCCCTCCCCACCCCCAATTTTGTATTTATTTATTTTTTAATTATTTTGT | |
| 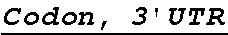 | GCAGCGATGGGGGCGGGGGGGGGGGGGGGGCGCGCGCCAGGCG | |
| 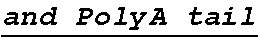 | GGGCGGGGCGGGGCGAGGGGCGGGGCGGGGCGAGGCGGAGAGG | |
| | TGCGGCGGCAGCCAATCAGAGCGGCGCGCTCCGAAAGTTTCCTTTT | |
| | ATGGCGAGGCGGCGGCGGCGGCGGCCCTATAAAAAGCGAAGCGCG | |
| | CGGCGGGCGGGAGTCGCTGCGCGCTGCCTTCGCCCCGTGCCCCGC | |
| | TCCGCCGCCGCCTCGCGCCGCCCGCCCCGGCTCTGACTGACCGCG | |
| | TTACTCCCACAGGTGAGCGGGCGGGACGGCCCTTCTCCTCCGGGCT | |
| | GTAATTAGCGCTTGGTTTAATGACGGCTTGTTTCTTTTCTGTGGCTGC | |
| | GTGAAAGCCTTGAGGGGCTCCGGGAGGGCCCTTTGTGCGGGGGGA | |
| | GCGGCTCGGGGGGTGCGTGCGTGTGTGTGTGCGTGGGGAGCGCCG | |
| | CGTGCGGCTCCGCGCTGCCCGGCGGCTGTGAGCGCTGCGGGCGCG | |
| | GCGCGGGGCTTTGTGCGCTCCGCAGTGTGCGCGAGGGGAGCGCGG | |
| | CCGGGGGCGGTGCCCCGCGGTGCGGGGGGGGCTGCGAGGGGAAC | |
| | AAAGGCTGCGTGCGGGGTGTGTGCGTGGGGGGGTGAGCAGGGGGT | |
| | GTGGGCGCGTCGGTCGGGCTGCAACCCCCCCTGCACCCCCCTCCC | |
| | CGAGTTGCTGAGCACGGCCCGGCTTCGGGTGCGGGGCTCCGTACG | |
| | GGGCGTGGCGCGGGGCTCGCCGTGCCGGGCGGGGGGTGGCGGCA | |
| | GGTGGGGGTGCCGGGCGGGGCGGGGCCGCCTCGGGCCGGGGAG | |
| | GGCTCGGGGGAGGGGCGCGGCGGCCCCCGGAGCGCCGGCGGCTG | |
| | TCGAGGCGCGGCGAGCCGCAGCCATTGCCTTTTATGGTAATCGTGC | |
| | GAGAGGGCGCAGGGACTTCCTTTGTCCCAAATCTGTGCGGAGCCGA | |
| | AATCTGGGAGGCGCCGCCGCACCCCCTCTAGCGGGCGCGGGGCGA | |
| | AGCGGTGCGGCGCCGGCAGGAAGGAAATGGGCGGGGAGGGCCTTC | |
| | GTGCGTCGCCGCGCCGCCGTCCCCTTCTCCCTCTCCAGCCTCGGGG | |
| | CTGTCCGCGGGGGGACGGCTGCCTTCGGGGGGGACGGGGCAGGG | |
| | CGGGGTTCGGCTTCTGGCGTGTGACCGGCGGCTCTAGAGCCTCTGC | |
| | TAACCATGTTCATGCCTTCTTCTTTTTCCTACAGCTCCTGGGCAACGT | |
| | GCTGGTTATTGTGCTGTCTCATCATTTTGGCAAAGAATTGGAGAATAA | |
| | ACTAGTATTCTTCTGGTCCCCACAGACTCAGAGAGAACCCGCCACCA | |
| | TGTTCGTGTTCCTGGTGCTGCTGCCCCTGGTGAGCAGCCAGTGCGT | |
| | GAACCTGACCACCAGGACCCAGCTGCCCCCCGCCTACACCAACAGC | |
| | TTCACCAGGGGCGTGTACTACCCCGACAAGGTGTTCAGGAGCAGCG | |
| | TGCTGCACAGCACCCAGGACCTGTTCCTGCCCTTCTTCAGCAACGTG | |
| | ACCTGGTTCCACGCCATCCACGTGAGCGGCACCAACGGCACCAAGA | |
| | GGTTCGACAACCCCGTGCTGCCCTTCAACGACGGCGTGTACTTCGC | |
| | CAGCACCGAGAAGAGCAACATCATCAGGGGCTGGATCTTCGGCACC | |
| | ACCCTGGACAGCAAGACCCAGAGCCTGCTGATCGTGAACAACGCCA | |
| | CCAACGTGGTGATCAAGGTGTGCGAGTTCCAGTTCTGCAACGACCC | |
| | CTTCCTGGGCGTGTACTACCACAAGAACAACAAGAGCTGGATGGAGA | |
| | GCGAGTTCAGGGTGTACAGCAGCGCCAACAACTGCACCTTCGAGTA | |
| | CGTGAGCCAGCCCTTCCTGATGGACCTGGAGGGCAAGCAGGGCAAC | |
| | TTCAAGAACCTGAGGGAGTTCGTGTTCAAGAACATCGACGGCTACTT | |
| | CAAGATCTACAGCAAGCACACCCCCATCAACCTGGTGAGGGACCTG | |
| | CCCCAGGGCTTCAGCGCCCTGGAGCCCCTGGTGGACCTGCCCATCG | |
| | GCATCAACATCACCAGGTTCCAGACCCTGCTGGCCCTGCACAGGAG | |
| | CTACCTGACCCCCGGCGACAGCAGCAGCGGCTGGACCGCCGGCGC | |
| | CGCCGCCTACTACGTGGGCTACCTGCAGCCCAGGACCTTCCTGCTG | |
| | AAGTACAACGAGAACGGCACCATCACCGACGCCGTGGACTGCGCCC | |
| | TGGACCCCCTGAGCGAGACCAAGTGCACCCTGAAGAGCTTCACCGT | |
| | GGAGAAGGGCATCTACCAGACCAGCAACTTCAGGGTGCAGCCCACC | |
| | GAGAGCATCGTGAGGTTCCCCAACATCACCAACCTGTGCCCCTTCG | |
| | GCGAGGTGTTCAACGCCACCAGGTTCGCCAGCGTGTACGCCTGGAA | |
| | CAGGAAGAGGATCAGCAACTGCGTGGCCGACTACAGCGTGCTGTAC | |
| | AACAGCGCCAGCTTCAGCACCTTCAAGTGCTACGGCGTGAGCCCCA | |
| | CCAAGCTGAACGACCTGTGCTTCACCAACGTGTACGCCGACAGCTTC | |
| | GTGATCAGGGGCGACGAGGTGAGGCAGATCGCCCCCGGCCAGACC | |
| | GGCAAGATCGCCGACTACAACTACAAGCTGCCCGACGACTTCACCG | |
| | GCTGCGTGATCGCCTGGAACAGCAACAACCTGGACAGCAAGGTGGG | |
| | CGGCAACTACAACTACCTGTACAGGCTGTTCAGGAAGAGCAACCTGA | |
| | AGCCCTTCGAGAGGGACATCAGCACCGAGATCTACCAGGCCGGCAG | |
| | CACCCCCTGCAACGGCGTGGAGGGCTTCAACTGCTACTTCCCCCTG | |
| | CAGAGCTACGGCTTCCAGCCCACCAACGGCGTGGGCTACCAGCCCT | |
| | ACAGGGTGGTGGTGCTGAGCTTCGAGCTGCTGCACGCCCCCGCCAC | |
| | CGTGTGCGGCCCCAAGAAGAGCACCAACCTGGTGAAGAACAAGTGC | |
| | GTGAACTTCAACTTCAACGGCCTGACCGGCACCGGCGTGCTGACCG | |
| | AGAGCAACAAGAAGTTCCTGCCCTTCCAGCAGTTCGGCAGGGACAT | |
| | CGCCGACACCACCGACGCCGTGAGGGACCCCCAGACCCTGGAGAT | |
| | CCTGGACATCACCCCCTGCAGCTTCGGCGGCGTGAGCGTGATCACC | |
| | CCCGGCACCAACACCAGCAACCAGGTGGCCGTGCTGTACCAGGACG | |
| | TGAACTGCACCGAGGTGCCCGTGGCCATCCACGCCGACCAGCTGAC | |
| | CCCCACCTGGAGGGTGTACAGCACCGGCAGCAACGTGTTCCAGACC | |
| | AGGGCCGGCTGCCTGATCGGCGCCGAGCACGTGAACAACAGCTAC | |
| | GAGTGCGACATCCCCATCGGCGCCGGCATCTGCGCCAGCTACCAGA | |
| | CCCAGACCAACAGCCCCGGCAGCGCCAGCAGCGTGGCCAGCCAGA | |
| | GCATCATCGCCTACACCATGAGCCTGGGCGCCGAGAACAGCGTGGC | |
| | CTACAGCAACAACAGCATCGCCATCCCCACCAACTTCACCATCAGCG | |
| | TGACCACCGAGATCCTGCCCGTGAGCATGACCAAGACCAGCGTGGA | |
| | CTGCACCATGTACATCTGCGGCGACAGCACCGAGTGCAGCAACCTG | |
| | CTGCTGCAGTACGGCAGCTTCTGCACCCAGCTGAACAGGGCCCTGA | |
| | CCGGCATCGCCGTGGAGCAGGACAAGAACACCCAGGAGGTGTTCGC | |
| | CCAGGTGAAGCAGATCTACAAGACCCCCCCCATCAAGGACTTCGGC | |
| | GGCTTCAACTTCAGCCAGATCCTGCCCGACCCCAGCAAGCCCAGCA | |
| | AGAGGAGCCCCATCGAGGACCTGCTGTTCAACAAGGTGACCCTGGC | |
| | CGACGCCGGCTTCATCAAGCAGTACGGCGACTGCCTGGGCGACATC | |
| | GCCGCCAGGGACCTGATCTGCGCCCAGAAGTTCAACGGCCTGACCG | |
| | TGCTGCCCCCCCTGCTGACCGACGAGATGATCGCCCAGTACACCAG | |
| | CGCCCTGCTGGCCGGCACCATCACCAGCGGCTGGACCTTCGGCGC | |
| | CGGCCCCGCCCTGCAGATCCCCTTCCCCATGCAGATGGCCTACAGG | |
| | TTCAACGGCATCGGCGTGACCCAGAACGTGCTGTACGAGAACCAGA | |
| | AGCTGATCGCCAACCAGTTCAACAGCGCCATCGGCAAGATCCAGGA | |
| | CAGCCTGAGCAGCACCCCCAGCGCCCTGGGCAAGCTGCAGGACGT | |
| | GGTGAACCAGAACGCCCAGGCCCTGAACACCCTGGTGAAGCAGCTG | |
| | AGCAGCAACTTCGGCGCCATCAGCAGCGTGCTGAACGACATCCTGA | |
| | GCAGGCTGGACCCCCCCGAGGCCGAGGTGCAGATCGACAGGCTGA | |
| | TCACCGGCAGGCTGCAGAGCCTGCAGACCTACGTGACCCAGCAGCT | |
| | GATCAGGGCCGCCGAGATCAGGGCCAGCGCCAACCTGGCCGCCAC | |
| | CAAGATGAGCGAGTGCGTGCTGGGCCAGAGCAAGAGGGTGGACTTC | |
| | TGCGGCAAGGGCTACCACCTGATGAGCTTCCCCCAGAGCGCCCCCC | |
| | ACGGCGTGGTGTTCCTGCACGTGACCTACGTGCCCGCCCAGGAGAA | |
| | GAACTTCACCACCGCCCCCGCCATCTGCCACGACGGCAAGGCCCAC | |
| | TTCCCCAGGGAGGGCGTGTTCGTGAGCAACGGCACCCACTGGTTCG | |
| | TGACCCAGAGGAACTTCTACGAGCCCCAGATCATCACCACCGACAAC | |
| | ACCTTCGTGAGCGGCAACTGCGACGTGGTGATCGGCATCGTGAACA | |
| | ACACCGTGTACGACCCCCTGCAGCCCGAGCTGGACAGCTTCAAGGA | |
| | GGAGCTGGACAAGTACTTCAAGAACCACACCAGCCCCGACGTGGAC | |
| | CTGGGCGACATCAGCGGCATCAACGCCAGCGTGGTGAACATCCAGA | |
| | AGGAGATCGACAGGCTGAACGAGGTGGCCAAGAACCTGAACGAGAG | |
| | CCTGATCGACCTGCAGGAGCTGGGCAAGTACGAGCAGTACATCAAG | |
| | TGGCCCTGGTACATCTGGCTGGGCTTCATCGCCGGCCTGATCGCCA | |
| | TCGTGATGGTGACCATCATGCTGTGCTGCATGACCAGCTGCTGCAGC | |
| | TGCCTGAAGGGCTGCTGCAGCTGCGGCAGCTGCTGCAAGTTCGACG | |
| | AGGACGACAGCGAGCCCGTGCTGAAGGGCGTGAAGCTGCACTACAC | |
| | CGGAAGCGGAGCCACGAACTTCTCTCTGTTAAAGCAAGCAGGAGAT | |
| | 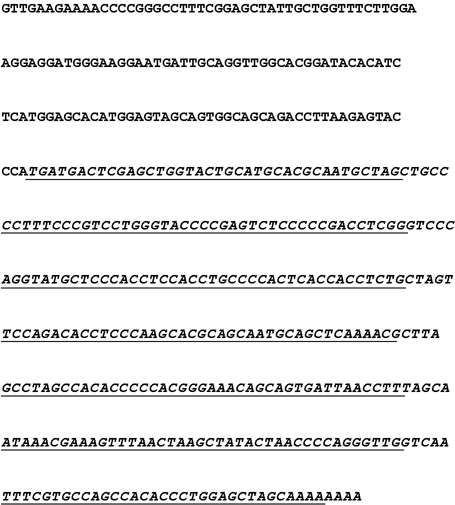 | |
| 4 | CTCGACATTGATTATTGACTAGTTATTAATAGTAATCAATTACGGGGT | 392 |
| promoter, | CATTAGTTCATAGCCCATATATGGAGTTCCGCGTTACATAACTTACGG | |
| 5’UTR and | TAAATGGCCCGCCTGGCTGACCGCCCAACGACCCCCGCCCATTGAC | |
| leader | GTCAATAATGACGTATGTTCCCATAGTAACGCCAATAGGGACTTTCCA | |
| sequence. | TTGACGTCAATGGGTGGAGTATTTACGGTAAACTGCCCACTTGGCAG | |
| Spike | TACATCAAGTGTATCATATGCCAAGTACGCCCCCTATTGACGTCAATG | |
| glycoprotein | ACGGTAAATGGCCCGCCTGGCATTATGCCCAGTACATGACCTTATGG | |
| (two proline | GACTTTCCTACTTGGCAGTACATCTACGTATTAGTCATCGCTATTACC | |
| substitutions), | ATGGTCGAGGTGAGCCCCACGTTCTGCTTCACTCTCCCCATCTCCCC | |
| 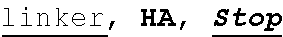 | CCCCTCCCCACCCCCAATTTTGTATTTATTTATTTTTTAATTATTTTGT | |
| 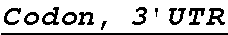 | GCAGCGATGGGGGCGGGGGGGGGGGGGGGGCGCGCGCCAGGCG | |
| 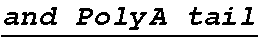 | GGGCGGGGCGGGGCGAGGGGCGGGGCGGGGCGAGGCGGAGAGG | |
| | TGCGGCGGCAGCCAATCAGAGCGGCGCGCTCCGAAAGTTTCCTTTT | |
| | ATGGCGAGGCGGCGGCGGCGGCGGCCCTATAAAAAGCGAAGCGCG | |
| | CGGCGGGCGGGAGTCGCTGCGCGCTGCCTTCGCCCCGTGCCCCGC | |
| | TCCGCCGCCGCCTCGCGCCGCCCGCCCCGGCTCTGACTGACCGCG | |
| | TTACTCCCACAGGTGAGCGGGCGGGACGGCCCTTCTCCTCCGGGCT | |
| | GTAATTAGCGCTTGGTTTAATGACGGCTTGTTTCTTTTCTGTGGCTGC | |
| | GTGAAAGCCTTGAGGGGCTCCGGGAGGGCCCTTTGTGCGGGGGGA | |
| | GCGGCTCGGGGGGTGCGTGCGTGTGTGTGTGCGTGGGGAGCGCCG | |
| | CGTGCGGCTCCGCGCTGCCCGGCGGCTGTGAGCGCTGCGGGCGCG | |
| | GCGCGGGGCTTTGTGCGCTCCGCAGTGTGCGCGAGGGGAGCGCGG | |
| | CCGGGGGCGGTGCCCCGCGGTGCGGGGGGGGCTGCGAGGGGAAC | |
| | AAAGGCTGCGTGCGGGGTGTGTGCGTGGGGGGGTGAGCAGGGGGT | |
| | GTGGGCGCGTCGGTCGGGCTGCAACCCCCCCTGCACCCCCCTCCC | |
| | CGAGTTGCTGAGCACGGCCCGGCTTCGGGTGCGGGGCTCCGTACG | |
| | GGGCGTGGCGCGGGGCTCGCCGTGCCGGGCGGGGGGTGGCGGCA | |
| | GGTGGGGGTGCCGGGCGGGGCGGGGCCGCCTCGGGCCGGGGAG | |
| | GGCTCGGGGGAGGGGCGCGGCGGCCCCCGGAGCGCCGGCGGCTG | |
| | TCGAGGCGCGGCGAGCCGCAGCCATTGCCTTTTATGGTAATCGTGC | |
| | GAGAGGGCGCAGGGACTTCCTTTGTCCCAAATCTGTGCGGAGCCGA | |
| | AATCTGGGAGGCGCCGCCGCACCCCCTCTAGCGGGCGCGGGGCGA | |
| | AGCGGTGCGGCGCCGGCAGGAAGGAAATGGGCGGGGAGGGCCTTC | |
| | GTGCGTCGCCGCGCCGCCGTCCCCTTCTCCCTCTCCAGCCTCGGGG | |
| | CTGTCCGCGGGGGGACGGCTGCCTTCGGGGGGGACGGGGCAGGG | |
| | CGGGGTTCGGCTTCTGGCGTGTGACCGGCGGCTCTAGAGCCTCTGC | |
| | TAACCATGTTCATGCCTTCTTCTTTTTCCTACAGCTCCTGGGCAACGT | |
| | GCTGGTTATTGTGCTGTCTCATCATTTTGGCAAAGAATTGGAGAATAA | |
| | ACTAGTATTCTTCTGGTCCCCACAGACTCAGAGAGAACCCGCCACCA | |
| | TGTTCGTGTTCCTGGTGCTGCTGCCCCTGGTGAGCAGCCAGTGCGT | |
| | GAACCTGACCACCAGGACCCAGCTGCCCCCCGCCTACACCAACAGC | |
| | TTCACCAGGGGCGTGTACTACCCCGACAAGGTGTTCAGGAGCAGCG | |
| | TGCTGCACAGCACCCAGGACCTGTTCCTGCCCTTCTTCAGCAACGTG | |
| | ACCTGGTTCCACGCCATCCACGTGAGCGGCACCAACGGCACCAAGA | |
| | GGTTCGACAACCCCGTGCTGCCCTTCAACGACGGCGTGTACTTCGC | |
| | CAGCACCGAGAAGAGCAACATCATCAGGGGCTGGATCTTCGGCACC | |
| | ACCCTGGACAGCAAGACCCAGAGCCTGCTGATCGTGAACAACGCCA | |
| | CCAACGTGGTGATCAAGGTGTGCGAGTTCCAGTTCTGCAACGACCC | |
| | CTTCCTGGGCGTGTACTACCACAAGAACAACAAGAGCTGGATGGAGA | |
| | GCGAGTTCAGGGTGTACAGCAGCGCCAACAACTGCACCTTCGAGTA | |
| | CGTGAGCCAGCCCTTCCTGATGGACCTGGAGGGCAAGCAGGGCAAC | |
| | TTCAAGAACCTGAGGGAGTTCGTGTTCAAGAACATCGACGGCTACTT | |
| | CAAGATCTACAGCAAGCACACCCCCATCAACCTGGTGAGGGACCTG | |
| | CCCCAGGGCTTCAGCGCCCTGGAGCCCCTGGTGGACCTGCCCATCG | |
| | GCATCAACATCACCAGGTTCCAGACCCTGCTGGCCCTGCACAGGAG | |
| | CTACCTGACCCCCGGCGACAGCAGCAGCGGCTGGACCGCCGGCGC | |
| | CGCCGCCTACTACGTGGGCTACCTGCAGCCCAGGACCTTCCTGCTG | |
| | AAGTACAACGAGAACGGCACCATCACCGACGCCGTGGACTGCGCCC | |
| | TGGACCCCCTGAGCGAGACCAAGTGCACCCTGAAGAGCTTCACCGT | |
| | GGAGAAGGGCATCTACCAGACCAGCAACTTCAGGGTGCAGCCCACC | |
| | GAGAGCATCGTGAGGTTCCCCAACATCACCAACCTGTGCCCCTTCG | |
| | GCGAGGTGTTCAACGCCACCAGGTTCGCCAGCGTGTACGCCTGGAA | |
| | CAGGAAGAGGATCAGCAACTGCGTGGCCGACTACAGCGTGCTGTAC | |
| | AACAGCGCCAGCTTCAGCACCTTCAAGTGCTACGGCGTGAGCCCCA | |
| | CCAAGCTGAACGACCTGTGCTTCACCAACGTGTACGCCGACAGCTTC | |
| | GTGATCAGGGGCGACGAGGTGAGGCAGATCGCCCCCGGCCAGACC | |
| | GGCAAGATCGCCGACTACAACTACAAGCTGCCCGACGACTTCACCG | |
| | GCTGCGTGATCGCCTGGAACAGCAACAACCTGGACAGCAAGGTGGG | |
| | CGGCAACTACAACTACCTGTACAGGCTGTTCAGGAAGAGCAACCTGA | |
| | AGCCCTTCGAGAGGGACATCAGCACCGAGATCTACCAGGCCGGCAG | |
| | CACCCCCTGCAACGGCGTGGAGGGCTTCAACTGCTACTTCCCCCTG | |
| | CAGAGCTACGGCTTCCAGCCCACCAACGGCGTGGGCTACCAGCCCT | |
| | ACAGGGTGGTGGTGCTGAGCTTCGAGCTGCTGCACGCCCCCGCCAC | |
| | CGTGTGCGGCCCCAAGAAGAGCACCAACCTGGTGAAGAACAAGTGC | |
| | GTGAACTTCAACTTCAACGGCCTGACCGGCACCGGCGTGCTGACCG | |
| | AGAGCAACAAGAAGTTCCTGCCCTTCCAGCAGTTCGGCAGGGACAT | |
| | CGCCGACACCACCGACGCCGTGAGGGACCCCCAGACCCTGGAGAT | |
| | CCTGGACATCACCCCCTGCAGCTTCGGCGGCGTGAGCGTGATCACC | |
| | CCCGGCACCAACACCAGCAACCAGGTGGCCGTGCTGTACCAGGACG | |
| | TGAACTGCACCGAGGTGCCCGTGGCCATCCACGCCGACCAGCTGAC | |
| | CCCCACCTGGAGGGTGTACAGCACCGGCAGCAACGTGTTCCAGACC | |
| | AGGGCCGGCTGCCTGATCGGCGCCGAGCACGTGAACAACAGCTAC | |
| | GAGTGCGACATCCCCATCGGCGCCGGCATCTGCGCCAGCTACCAGA | |
| | CCCAGACCAACAGCCCCAGGAGGGCCAGGAGCGTGGCCAGCCAGA | |
| | GCATCATCGCCTACACCATGAGCCTGGGCGCCGAGAACAGCGTGGC | |
| | CTACAGCAACAACAGCATCGCCATCCCCACCAACTTCACCATCAGCG | |
| | TGACCACCGAGATCCTGCCCGTGAGCATGACCAAGACCAGCGTGGA | |
| | CTGCACCATGTACATCTGCGGCGACAGCACCGAGTGCAGCAACCTG | |
| | CTGCTGCAGTACGGCAGCTTCTGCACCCAGCTGAACAGGGCCCTGA | |
| | CCGGCATCGCCGTGGAGCAGGACAAGAACACCCAGGAGGTGTTCGC | |
| | CCAGGTGAAGCAGATCTACAAGACCCCCCCCATCAAGGACTTCGGC | |
| | GGCTTCAACTTCAGCCAGATCCTGCCCGACCCCAGCAAGCCCAGCA | |
| | AGAGGAGCTTCATCGAGGACCTGCTGTTCAACAAGGTGACCCTGGC | |
| | CGACGCCGGCTTCATCAAGCAGTACGGCGACTGCCTGGGCGACATC | |
| | GCCGCCAGGGACCTGATCTGCGCCCAGAAGTTCAACGGCCTGACCG | |
| | TGCTGCCCCCCCTGCTGACCGACGAGATGATCGCCCAGTACACCAG | |
| | CGCCCTGCTGGCCGGCACCATCACCAGCGGCTGGACCTTCGGCGC | |
| | CGGCGCCGCCCTGCAGATCCCCTTCGCCATGCAGATGGCCTACAGG | |
| | TTCAACGGCATCGGCGTGACCCAGAACGTGCTGTACGAGAACCAGA | |
| | AGCTGATCGCCAACCAGTTCAACAGCGCCATCGGCAAGATCCAGGA | |
| | CAGCCTGAGCAGCACCGCCAGCGCCCTGGGCAAGCTGCAGGACGT | |
| | GGTGAACCAGAACGCCCAGGCCCTGAACACCCTGGTGAAGCAGCTG | |
| | AGCAGCAACTTCGGCGCCATCAGCAGCGTGCTGAACGACATCCTGA | |
| | GCAGGCTGGACCCCCCCGAGGCCGAGGTGCAGATCGACAGGCTGA | |
| | TCACCGGCAGGCTGCAGAGCCTGCAGACCTACGTGACCCAGCAGCT | |
| | GATCAGGGCCGCCGAGATCAGGGCCAGCGCCAACCTGGCCGCCAC | |
| | CAAGATGAGCGAGTGCGTGCTGGGCCAGAGCAAGAGGGTGGACTTC | |
| | TGCGGCAAGGGCTACCACCTGATGAGCTTCCCCCAGAGCGCCCCCC | |
| | ACGGCGTGGTGTTCCTGCACGTGACCTACGTGCCCGCCCAGGAGAA | |
| | GAACTTCACCACCGCCCCCGCCATCTGCCACGACGGCAAGGCCCAC | |
| | TTCCCCAGGGAGGGCGTGTTCGTGAGCAACGGCACCCACTGGTTCG | |
| | TGACCCAGAGGAACTTCTACGAGCCCCAGATCATCACCACCGACAAC | |
| | ACCTTCGTGAGCGGCAACTGCGACGTGGTGATCGGCATCGTGAACA | |
| | ACACCGTGTACGACCCCCTGCAGCCCGAGCTGGACAGCTTCAAGGA | |
| | GGAGCTGGACAAGTACTTCAAGAACCACACCAGCCCCGACGTGGAC | |
| | CTGGGCGACATCAGCGGCATCAACGCCAGCGTGGTGAACATCCAGA | |
| | AGGAGATCGACAGGCTGAACGAGGTGGCCAAGAACCTGAACGAGAG | |
| | CCTGATCGACCTGCAGGAGCTGGGCAAGTACGAGCAGTACATCAAG | |
| | TGGCCCTGGTACATCTGGCTGGGCTTCATCGCCGGCCTGATCGCCA | |
| | TCGTGATGGTGACCATCATGCTGTGCTGCATGACCAGCTGCTGCAGC | |
| | TGCCTGAAGGGCTGCTGCAGCTGCGGCAGCTGCTGCAAGTTCGACG | |
| | AGGACGACAGCGAGCCCGTGCTGAAGGGCGTGAAGCTGCACTACAC | |
| | CGGAAGCGGAGCCACGAACTTCTCTCTGTTAAAGCAAGCAGGAGAT | |
| | 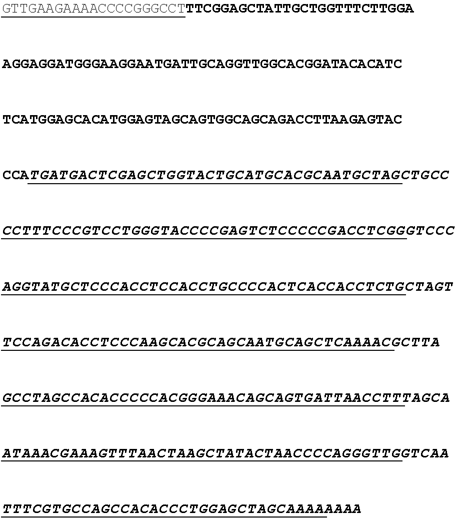 | |
| 5 | ATGTTCGTGTTCCTGGTGCTGCTGCCCCTGGTGAGCAGCCAGTGCG | 393 |
| leader | TGAACCTGACCACCAGGACCCAGCTGCCCCCCGCCTACACCAACAG | |
| sequence, | CTTCACCAGGGGCGTGTACTACCCCGACAAGGTGTTCAGGAGCAGC | |
| Spike | GTGCTGCACAGCACCCAGGACCTGTTCCTGCCCTTCTTCAGCAACGT | |
| glycoprotein | GACCTGGTTCCACGCCATCCACGTGAGCGGCACCAACGGCACCAAG | |
| (HexaPro- | AGGTTCGACAACCCCGTGCTGCCCTTCAACGACGGCGTGTACTTCG | |
| mutations), | CCAGCACCGAGAAGAGCAACATCATCAGGGGCTGGATCTTCGGCAC | |
| 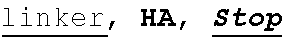 | CACCCTGGACAGCAAGACCCAGAGCCTGCTGATCGTGAACAACGCC | |
| 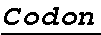 | ACCAACGTGGTGATCAAGGTGTGCGAGTTCCAGTTCTGCAACGACCC | |
| | CTTCCTGGGCGTGTACTACCACAAGAACAACAAGAGCTGGATGGAGA | |
| | GCGAGTTCAGGGTGTACAGCAGCGCCAACAACTGCACCTTCGAGTA | |
| | CGTGAGCCAGCCCTTCCTGATGGACCTGGAGGGCAAGCAGGGCAAC | |
| | TTCAAGAACCTGAGGGAGTTCGTGTTCAAGAACATCGACGGCTACTT | |
| | CAAGATCTACAGCAAGCACACCCCCATCAACCTGGTGAGGGACCTG | |
| | CCCCAGGGCTTCAGCGCCCTGGAGCCCCTGGTGGACCTGCCCATCG | |
| | GCATCAACATCACCAGGTTCCAGACCCTGCTGGCCCTGCACAGGAG | |
| | CTACCTGACCCCCGGCGACAGCAGCAGCGGCTGGACCGCCGGCGC | |
| | CGCCGCCTACTACGTGGGCTACCTGCAGCCCAGGACCTTCCTGCTG | |
| | AAGTACAACGAGAACGGCACCATCACCGACGCCGTGGACTGCGCCC | |
| | TGGACCCCCTGAGCGAGACCAAGTGCACCCTGAAGAGCTTCACCGT | |
| | GGAGAAGGGCATCTACCAGACCAGCAACTTCAGGGTGCAGCCCACC | |
| | GAGAGCATCGTGAGGTTCCCCAACATCACCAACCTGTGCCCCTTCG | |
| | GCGAGGTGTTCAACGCCACCAGGTTCGCCAGCGTGTACGCCTGGAA | |
| | CAGGAAGAGGATCAGCAACTGCGTGGCCGACTACAGCGTGCTGTAC | |
| | AACAGCGCCAGCTTCAGCACCTTCAAGTGCTACGGCGTGAGCCCCA | |
| | CCAAGCTGAACGACCTGTGCTTCACCAACGTGTACGCCGACAGCTTC | |
| | GTGATCAGGGGCGACGAGGTGAGGCAGATCGCCCCCGGCCAGACC | |
| | GGCAAGATCGCCGACTACAACTACAAGCTGCCCGACGACTTCACCG | |
| | GCTGCGTGATCGCCTGGAACAGCAACAACCTGGACAGCAAGGTGGG | |
| | CGGCAACTACAACTACCTGTACAGGCTGTTCAGGAAGAGCAACCTGA | |
| | AGCCCTTCGAGAGGGACATCAGCACCGAGATCTACCAGGCCGGCAG | |
| | CACCCCCTGCAACGGCGTGGAGGGCTTCAACTGCTACTTCCCCCTG | |
| | CAGAGCTACGGCTTCCAGCCCACCAACGGCGTGGGCTACCAGCCCT | |
| | ACAGGGTGGTGGTGCTGAGCTTCGAGCTGCTGCACGCCCCCGCCAC | |
| | CGTGTGCGGCCCCAAGAAGAGCACCAACCTGGTGAAGAACAAGTGC | |
| | GTGAACTTCAACTTCAACGGCCTGACCGGCACCGGCGTGCTGACCG | |
| | AGAGCAACAAGAAGTTCCTGCCCTTCCAGCAGTTCGGCAGGGACAT | |
| | CGCCGACACCACCGACGCCGTGAGGGACCCCCAGACCCTGGAGAT | |
| | CCTGGACATCACCCCCTGCAGCTTCGGCGGCGTGAGCGTGATCACC | |
| | CCCGGCACCAACACCAGCAACCAGGTGGCCGTGCTGTACCAGGACG | |
| | TGAACTGCACCGAGGTGCCCGTGGCCATCCACGCCGACCAGCTGAC | |
| | CCCCACCTGGAGGGTGTACAGCACCGGCAGCAACGTGTTCCAGACC | |
| | AGGGCCGGCTGCCTGATCGGCGCCGAGCACGTGAACAACAGCTAC | |
| | GAGTGCGACATCCCCATCGGCGCCGGCATCTGCGCCAGCTACCAGA | |
| | CCCAGACCAACAGCCCCGGCAGCGCCAGCAGCGTGGCCAGCCAGA | |
| | GCATCATCGCCTACACCATGAGCCTGGGCGCCGAGAACAGCGTGGC | |
| | CTACAGCAACAACAGCATCGCCATCCCCACCAACTTCACCATCAGCG | |
| | TGACCACCGAGATCCTGCCCGTGAGCATGACCAAGACCAGCGTGGA | |
| | CTGCACCATGTACATCTGCGGCGACAGCACCGAGTGCAGCAACCTG | |
| | CTGCTGCAGTACGGCAGCTTCTGCACCCAGCTGAACAGGGCCCTGA | |
| | CCGGCATCGCCGTGGAGCAGGACAAGAACACCCAGGAGGTGTTCGC | |
| | CCAGGTGAAGCAGATCTACAAGACCCCCCCCATCAAGGACTTCGGC | |
| | GGCTTCAACTTCAGCCAGATCCTGCCCGACCCCAGCAAGCCCAGCA | |
| | AGAGGAGCCCCATCGAGGACCTGCTGTTCAACAAGGTGACCCTGGC | |
| | CGACGCCGGCTTCATCAAGCAGTACGGCGACTGCCTGGGCGACATC | |
| | GCCGCCAGGGACCTGATCTGCGCCCAGAAGTTCAACGGCCTGACCG | |
| | TGCTGCCCCCCCTGCTGACCGACGAGATGATCGCCCAGTACACCAG | |
| | CGCCCTGCTGGCCGGCACCATCACCAGCGGCTGGACCTTCGGCGC | |
| | CGGCCCCGCCCTGCAGATCCCCTTCCCCATGCAGATGGCCTACAGG | |
| | TTCAACGGCATCGGCGTGACCCAGAACGTGCTGTACGAGAACCAGA | |
| | AGCTGATCGCCAACCAGTTCAACAGCGCCATCGGCAAGATCCAGGA | |
| | CAGCCTGAGCAGCACCCCCAGCGCCCTGGGCAAGCTGCAGGACGT | |
| | GGTGAACCAGAACGCCCAGGCCCTGAACACCCTGGTGAAGCAGCTG | |
| | AGCAGCAACTTCGGCGCCATCAGCAGCGTGCTGAACGACATCCTGA | |
| | GCAGGCTGGACCCCCCCGAGGCCGAGGTGCAGATCGACAGGCTGA | |
| | TCACCGGCAGGCTGCAGAGCCTGCAGACCTACGTGACCCAGCAGCT | |
| | GATCAGGGCCGCCGAGATCAGGGCCAGCGCCAACCTGGCCGCCAC | |
| | CAAGATGAGCGAGTGCGTGCTGGGCCAGAGCAAGAGGGTGGACTTC | |
| | TGCGGCAAGGGCTACCACCTGATGAGCTTCCCCCAGAGCGCCCCCC | |
| | ACGGCGTGGTGTTCCTGCACGTGACCTACGTGCCCGCCCAGGAGAA | |
| | GAACTTCACCACCGCCCCCGCCATCTGCCACGACGGCAAGGCCCAC | |
| | TTCCCCAGGGAGGGCGTGTTCGTGAGCAACGGCACCCACTGGTTCG | |
| | TGACCCAGAGGAACTTCTACGAGCCCCAGATCATCACCACCGACAAC | |
| | ACCTTCGTGAGCGGCAACTGCGACGTGGTGATCGGCATCGTGAACA | |
| | ACACCGTGTACGACCCCCTGCAGCCCGAGCTGGACAGCTTCAAGGA | |
| | GGAGCTGGACAAGTACTTCAAGAACCACACCAGCCCCGACGTGGAC | |
| | CTGGGCGACATCAGCGGCATCAACGCCAGCGTGGTGAACATCCAGA | |
| | AGGAGATCGACAGGCTGAACGAGGTGGCCAAGAACCTGAACGAGAG | |
| | CCTGATCGACCTGCAGGAGCTGGGCAAGTACGAGCAGTACATCAAG | |
| | TGGCCCTGGTACATCTGGCTGGGCTTCATCGCCGGCCTGATCGCCA | |
| | TCGTGATGGTGACCATCATGCTGTGCTGCATGACCAGCTGCTGCAGC | |
| | TGCCTGAAGGGCTGCTGCAGCTGCGGCAGCTGCTGCAAGTTCGACG | |
| | AGGACGACAGCGAGCCCGTGCTGAAGGGCGTGAAGCTGCACTACAC | |
| | CGGAAGCGGAGCCACGAACTTCTCTCTGTTAAAGCAAGCAGGAGAT | |
| | 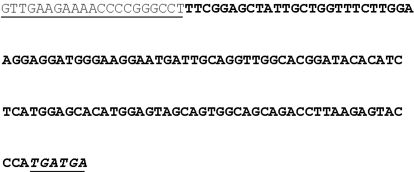 | |
| 6 | ATGTTCGTGTTCCTGGTGCTGCTGCCCCTGGTGAGCAGCCAGTGCG | 394 |
| leader | TGAACCTGACCACCAGGACCCAGCTGCCCCCCGCCTACACCAACAG | |
| sequence, | CTTCACCAGGGGCGTGTACTACCCCGACAAGGTGTTCAGGAGCAGC | |
| Spike | GTGCTGCACAGCACCCAGGACCTGTTCCTGCCCTTCTTCAGCAACGT | |
| glycoprotein | GACCTGGTTCCACGCCATCCACGTGAGCGGCACCAACGGCACCAAG | |
| (two proline | AGGTTCGACAACCCCGTGCTGCCCTTCAACGACGGCGTGTACTTCG | |
| substitutions) | CCAGCACCGAGAAGAGCAACATCATCAGGGGCTGGATCTTCGGCAC | |
| 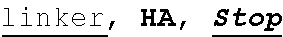 | CACCCTGGACAGCAAGACCCAGAGCCTGCTGATCGTGAACAACGCC | |
| 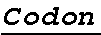 | ACCAACGTGGTGATCAAGGTGTGCGAGTTCCAGTTCTGCAACGACCC | |
| | CTTCCTGGGCGTGTACTACCACAAGAACAACAAGAGCTGGATGGAGA | |
| | GCGAGTTCAGGGTGTACAGCAGCGCCAACAACTGCACCTTCGAGTA | |
| | CGTGAGCCAGCCCTTCCTGATGGACCTGGAGGGCAAGCAGGGCAAC | |
| | TTCAAGAACCTGAGGGAGTTCGTGTTCAAGAACATCGACGGCTACTT | |
| | CAAGATCTACAGCAAGCACACCCCCATCAACCTGGTGAGGGACCTG | |
| | CCCCAGGGCTTCAGCGCCCTGGAGCCCCTGGTGGACCTGCCCATCG | |
| | GCATCAACATCACCAGGTTCCAGACCCTGCTGGCCCTGCACAGGAG | |
| | CTACCTGACCCCCGGCGACAGCAGCAGCGGCTGGACCGCCGGCGC | |
| | CGCCGCCTACTACGTGGGCTACCTGCAGCCCAGGACCTTCCTGCTG | |
| | AAGTACAACGAGAACGGCACCATCACCGACGCCGTGGACTGCGCCC | |
| | TGGACCCCCTGAGCGAGACCAAGTGCACCCTGAAGAGCTTCACCGT | |
| | GGAGAAGGGCATCTACCAGACCAGCAACTTCAGGGTGCAGCCCACC | |
| | GAGAGCATCGTGAGGTTCCCCAACATCACCAACCTGTGCCCCTTCG | |
| | GCGAGGTGTTCAACGCCACCAGGTTCGCCAGCGTGTACGCCTGGAA | |
| | CAGGAAGAGGATCAGCAACTGCGTGGCCGACTACAGCGTGCTGTAC | |
| | AACAGCGCCAGCTTCAGCACCTTCAAGTGCTACGGCGTGAGCCCCA | |
| | CCAAGCTGAACGACCTGTGCTTCACCAACGTGTACGCCGACAGCTTC | |
| | GTGATCAGGGGCGACGAGGTGAGGCAGATCGCCCCCGGCCAGACC | |
| | GGCAAGATCGCCGACTACAACTACAAGCTGCCCGACGACTTCACCG | |
| | GCTGCGTGATCGCCTGGAACAGCAACAACCTGGACAGCAAGGTGGG | |
| | CGGCAACTACAACTACCTGTACAGGCTGTTCAGGAAGAGCAACCTGA | |
| | AGCCCTTCGAGAGGGACATCAGCACCGAGATCTACCAGGCCGGCAG | |
| | CACCCCCTGCAACGGCGTGGAGGGCTTCAACTGCTACTTCCCCCTG | |
| | CAGAGCTACGGCTTCCAGCCCACCAACGGCGTGGGCTACCAGCCCT | |
| | ACAGGGTGGTGGTGCTGAGCTTCGAGCTGCTGCACGCCCCCGCCAC | |
| | CGTGTGCGGCCCCAAGAAGAGCACCAACCTGGTGAAGAACAAGTGC | |
| | GTGAACTTCAACTTCAACGGCCTGACCGGCACCGGCGTGCTGACCG | |
| | AGAGCAACAAGAAGTTCCTGCCCTTCCAGCAGTTCGGCAGGGACAT | |
| | CGCCGACACCACCGACGCCGTGAGGGACCCCCAGACCCTGGAGAT | |
| | CCTGGACATCACCCCCTGCAGCTTCGGCGGCGTGAGCGTGATCACC | |
| | CCCGGCACCAACACCAGCAACCAGGTGGCCGTGCTGTACCAGGACG | |
| | TGAACTGCACCGAGGTGCCCGTGGCCATCCACGCCGACCAGCTGAC | |
| | CCCCACCTGGAGGGTGTACAGCACCGGCAGCAACGTGTTCCAGACC | |
| | AGGGCCGGCTGCCTGATCGGCGCCGAGCACGTGAACAACAGCTAC | |
| | GAGTGCGACATCCCCATCGGCGCCGGCATCTGCGCCAGCTACCAGA | |
| | CCCAGACCAACAGCCCCAGGAGGGCCAGGAGCGTGGCCAGCCAGA | |
| | GCATCATCGCCTACACCATGAGCCTGGGCGCCGAGAACAGCGTGGC | |
| | CTACAGCAACAACAGCATCGCCATCCCCACCAACTTCACCATCAGCG | |
| | TGACCACCGAGATCCTGCCCGTGAGCATGACCAAGACCAGCGTGGA | |
| | CTGCACCATGTACATCTGCGGCGACAGCACCGAGTGCAGCAACCTG | |
| | CTGCTGCAGTACGGCAGCTTCTGCACCCAGCTGAACAGGGCCCTGA | |
| | CCGGCATCGCCGTGGAGCAGGACAAGAACACCCAGGAGGTGTTCGC | |
| | CCAGGTGAAGCAGATCTACAAGACCCCCCCCATCAAGGACTTCGGC | |
| | GGCTTCAACTTCAGCCAGATCCTGCCCGACCCCAGCAAGCCCAGCA | |
| | AGAGGAGCTTCATCGAGGACCTGCTGTTCAACAAGGTGACCCTGGC | |
| | CGACGCCGGCTTCATCAAGCAGTACGGCGACTGCCTGGGCGACATC | |
| | GCCGCCAGGGACCTGATCTGCGCCCAGAAGTTCAACGGCCTGACCG | |
| | TGCTGCCCCCCCTGCTGACCGACGAGATGATCGCCCAGTACACCAG | |
| | CGCCCTGCTGGCCGGCACCATCACCAGCGGCTGGACCTTCGGCGC | |
| | CGGCGCCGCCCTGCAGATCCCCTTCGCCATGCAGATGGCCTACAGG | |
| | TTCAACGGCATCGGCGTGACCCAGAACGTGCTGTACGAGAACCAGA | |
| | AGCTGATCGCCAACCAGTTCAACAGCGCCATCGGCAAGATCCAGGA | |
| | CAGCCTGAGCAGCACCGCCAGCGCCCTGGGCAAGCTGCAGGACGT | |
| | GGTGAACCAGAACGCCCAGGCCCTGAACACCCTGGTGAAGCAGCTG | |
| | AGCAGCAACTTCGGCGCCATCAGCAGCGTGCTGAACGACATCCTGA | |
| | GCAGGCTGGACCCCCCCGAGGCCGAGGTGCAGATCGACAGGCTGA | |
| | TCACCGGCAGGCTGCAGAGCCTGCAGACCTACGTGACCCAGCAGCT | |
| | GATCAGGGCCGCCGAGATCAGGGCCAGCGCCAACCTGGCCGCCAC | |
| | CAAGATGAGCGAGTGCGTGCTGGGCCAGAGCAAGAGGGTGGACTTC | |
| | TGCGGCAAGGGCTACCACCTGATGAGCTTCCCCCAGAGCGCCCCCC | |
| | ACGGCGTGGTGTTCCTGCACGTGACCTACGTGCCCGCCCAGGAGAA | |
| | GAACTTCACCACCGCCCCCGCCATCTGCCACGACGGCAAGGCCCAC | |
| | TTCCCCAGGGAGGGCGTGTTCGTGAGCAACGGCACCCACTGGTTCG | |
| | TGACCCAGAGGAACTTCTACGAGCCCCAGATCATCACCACCGACAAC | |
| | ACCTTCGTGAGCGGCAACTGCGACGTGGTGATCGGCATCGTGAACA | |
| | ACACCGTGTACGACCCCCTGCAGCCCGAGCTGGACAGCTTCAAGGA | |
| | GGAGCTGGACAAGTACTTCAAGAACCACACCAGCCCCGACGTGGAC | |
| | CTGGGCGACATCAGCGGCATCAACGCCAGCGTGGTGAACATCCAGA | |
| | AGGAGATCGACAGGCTGAACGAGGTGGCCAAGAACCTGAACGAGAG | |
| | CCTGATCGACCTGCAGGAGCTGGGCAAGTACGAGCAGTACATCAAG | |
| | TGGCCCTGGTACATCTGGCTGGGCTTCATCGCCGGCCTGATCGCCA | |
| | TCGTGATGGTGACCATCATGCTGTGCTGCATGACCAGCTGCTGCAGC | |
| | TGCCTGAAGGGCTGCTGCAGCTGCGGCAGCTGCTGCAAGTTCGACG | |
| | AGGACGACAGCGAGCCCGTGCTGAAGGGCGTGAAGCTGCACTACAC | |
| | CGGAAGCGGAGCCACGAACTTCTCTCTGTTAAAGCAAGCAGGAGAT | |
| | 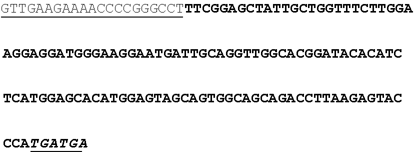 | |
| Pan-CoV-Flu | TAGTTATTAATAGTAATCAATTACGGGGTCATTAGTTCATAGCCCATAT | 395 |
| Vaccine #7 | ATGGAGTTCCGCGTTACATAACTTACGGTAAATGGCCCGCCTGGCTG | |
| CoV- Omicron | ACCGCCCAACGACCCCCGCCCATTGACGTCAATAATGACGTATGTTC | |
| BA.2 | CCATAGTAACGCCAATAGGGACTTTCCATTGACGTCAATGGGTGGAG | |
| Spike(HexaPro)- | TATTTACGGTAAACTGCCCACTTGGCAGTACATCAAGTGTATCATATG | |
| Influenza (HA) | CCAAGTACGCCCCCTATTGACGTCAATGACGGTAAATGGCCCGCCTG | |
| | GCATTATGCCCAGTACATGACCTTATGGGACTTTCCTACTTGGCAGTA | |
| | CATCTACGTATTAGTCATCGCTATTACCATGGTGATGCGGTTTTGGCA | |
| | GTACATCAATGGGCGTGGATAGCGGTTTGACTCACGGGGATTTCCAA | |
| | GTCTCCACCCCATTGACGTCAATGGGAGTTTGTTTTGGCACCAAAAT | |
| | CAACGGGACTTTCCAAAATGTCGTAACAACTCCGCCCCATTGACGCA | |
| | AATGGGCGGTAGGCGTGTACGGTGGGAGGTCTATATAAGCAGAGCT | |
| | GGTTTAGTGAACCGTCAGATCGAGAATAAACTAGTATTCTTCTGGTCC | |
| | CCACAGACTCAGAGAGAACCCGCCACCATGTTCGTGTTCCTGGTGCT | |
| | GCTGCCCCTGGTGAGCAGCCAGTGCGTGAACCTGATCACCAGGACC | |
| | CAGAGCTACACCAACAGCTTCACCAGGGGCGTGTACTACCCCGACA | |
| | AGGTGTTCAGGAGCAGCGTGCTGCACAGCACCCAGGACCTGTTCCT | |
| | GCCCTTCTTCAGCAACGTGACCTGGTTCCACGCCATCCACGTGAGC | |
| | GGCACCAACGGCACCAAGAGGTTCGACAACCCCGTGCTGCCCTTCA | |
| | ACGACGGCGTGTACTTCGCCAGCACCGAGAAGAGCAACATCATCAG | |
| | GGGCTGGATCTTCGGCACCACCCTGGACAGCAAGACCCAGAGCCTG | |
| | CTGATCGTGAACAACGCCACCAACGTGGTGATCAAGGTGTGCGAGTT | |
| | CCAGTTCTGCAACGACCCCTTCCTGGACGTGTACTACCACAAGAACA | |
| | ACAAGAGCTGGATGGAGAGCGAGTTCAGGGTGTACAGCAGCGCCAA | |
| | CAACTGCACCTTCGAGTACGTGAGCCAGCCCTTCCTGATGGACCTG | |
| | GAGGGCAAGCAGGGCAACTTCAAGAACCTGAGGGAGTTCGTGTTCA | |
| | AGAACATCGACGGCTACTTCAAGATCTACAGCAAGCACACCCCCATC | |
| | AACCTGGGCAGGGACCTGCCCCAGGGCTTCAGCGCCCTGGAGCCC | |
| | CTGGTGGACCTGCCCATCGGCATCAACATCACCAGGTTCCAGACCCT | |
| | GCTGGCCCTGCACAGGAGCTACCTGACCCCCGGCGACAGCAGCAG | |
| | CGGCTGGACCGCCGGCGCCGCCGCCTACTACGTGGGCTACCTGCA | |
| | GCCCAGGACCTTCCTGCTGAAGTACAACGAGAACGGCACCATCACC | |
| | GACGCCGTGGACTGCGCCCTGGACCCCCTGAGCGAGACCAAGTGC | |
| | ACCCTGAAGAGCTTCACCGTGGAGAAGGGCATCTACCAGACCAGCA | |
| | ACTTCAGGGTGCAGCCCACCGAGAGCATCGTGAGGTTCCCCAACAT | |
| | CACCAACCTGTGCCCCTTCGACGAGGTGTTCAACGCCACCAGGTTC | |
| | GCCAGCGTGTACGCCTGGAACAGGAAGAGGATCAGCAACTGCGTGG | |
| | CCGACTACAGCGTGCTGTACAACTTCGCCCCCTTCTTCGCCTTCAAG | |
| | TGCTACGGCGTGAGCCCCACCAAGCTGAACGACCTGTGCTTCACCA | |
| | ACGTGTACGCCGACAGCTTCGTGATCAGGGGCAACGAGGTGAGCCA | |
| | GATCGCCCCCGGCCAGACCGGCAACATCGCCGACTACAACTACAAG | |
| | CTGCCCGACGACTTCACCGGCTGCGTGATCGCCTGGAACAGCAACA | |
| | AGCTGGACAGCAAGGTGGGCGGCAACTACAACTACCTGTACAGGCT | |
| | GTTCAGGAAGAGCAACCTGAAGCCCTTCGAGAGGGACATCAGCACC | |
| | GAGATCTACCAGGCCGGCAACAAGCCCTGCAACGGCGTGGCCGGCT | |
| | TCAACTGCTACTTCCCCCTGAGGAGCTACGGCTTCAGGCCCACCTAC | |
| | GGCGTGGGCCACCAGCCCTACAGGGTGGTGGTGCTGAGCTTCGAG | |
| | CTGCTGCACGCCCCCGCCACCGTGTGCGGCCCCAAGAAGAGCACCA | |
| | ACCTGGTGAAGAACAAGTGCGTGAACTTCAACTTCAACGGCCTGACC | |
| | GGCACCGGCGTGCTGACCGAGAGCAACAAGAAGTTCCTGCCCTTCC | |
| | AGCAGTTCGGCAGGGACATCGCCGACACCACCGACGCCGTGAGGG | |
| | ACCCCCAGACCCTGGAGATCCTGGACATCACCCCCTGCAGCTTCGG | |
| | CGGCGTGAGCGTGATCACCCCCGGCACCAACACCAGCAACCAGGTG | |
| | GCCGTGCTGTACCAGGGCGTGAACTGCACCGAGGTGCCCGTGGCC | |
| | ATCCACGCCGACCAGCTGACCCCCACCTGGAGGGTGTACAGCACCG | |
| | GCAGCAACGTGTTCCAGACCAGGGCCGGCTGCCTGATCGGCGCCG | |
| | AGTACGTGAACAACAGCTACGAGTGCGACATCCCCATCGGCGCCGG | |
| | CATCTGCGCCAGCTACCAGACCCAGACCAAGAGCCACCAGCAGGCC | |
| | CAGAGCGTGGCCAGCCAGAGCATCATCGCCTACACCATGAGCCTGG | |
| | GCGCCGAGAACAGCGTGGCCTACAGCAACAACAGCATCGCCATCCC | |
| | CACCAACTTCACCATCAGCGTGACCACCGAGATCCTGCCCGTGAGC | |
| | ATGACCAAGACCAGCGTGGACTGCACCATGTACATCTGCGGCGACA | |
| | GCACCGAGTGCAGCAACCTGCTGCTGCAGTACGGCAGCTTCTGCAC | |
| | CCAGCTGAAGAGGGCCCTGACCGGCATCGCCGTGGAGCAGGACAA | |
| | GAACACCCAGGAGGTGTTCGCCCAGGTGAAGCAGATCTACAAGACC | |
| | CCCCCCATCAAGTACTTCGGCGGCTTCAACTTCAGCCAGATCCTGCC | |
| | CGACCCCAGCAAGCCCAGCAAGAGGAGCCCCATCGAGGACCTGCTG | |
| | TTCAACAAGGTGACCCTGGCCGACGCCGGCTTCATCAAGCAGTACG | |
| | GCGACTGCCTGGGCGACATCGCCGCCAGGGACCTGATCTGCGCCC | |
| | AGAAGTTCAACGGCCTGACCGTGCTGCCCCCCCTGCTGACCGACGA | |
| | GATGATCGCCCAGTACACCAGCGCCCTGCTGGCCGGCACCATCACC | |
| | AGCGGCTGGACCTTCGGCGCCGGCCCCGCCCTGCAGATCCCCTTCC | |
| | CCATGCAGATGGCCTACAGGTTCAACGGCATCGGCGTGACCCAGAA | |
| | CGTGCTGTACGAGAACCAGAAGCTGATCGCCAACCAGTTCAACAGC | |
| | GCCATCGGCAAGATCCAGGACAGCCTGAGCAGCACCCCCAGCGCCC | |
| | TGGGCAAGCTGCAGGACGTGGTGAACCACAACGCCCAGGCCCTGAA | |
| | CACCCTGGTGAAGCAGCTGAGCAGCAAGTTCGGCGCCATCAGCAGC | |
| | GTGCTGAACGACATCCTGAGCAGGCTGGACCCCCCCGAGGCCGAG | |
| | GTGCAGATCGACAGGCTGATCACCGGCAGGCTGCAGAGCCTGCAGA | |
| | CCTACGTGACCCAGCAGCTGATCAGGGCCGCCGAGATCAGGGCCAG | |
| | CGCCAACCTGGCCGCCACCAAGATGAGCGAGTGCGTGCTGGGCCA | |
| | GAGCAAGAGGGTGGACTTCTGCGGCAAGGGCTACCACCTGATGAGC | |
| | TTCCCCCAGAGCGCCCCCCACGGCGTGGTGTTCCTGCACGTGACCT | |
| | ACGTGCCCGCCCAGGAGAAGAACTTCACCACCGCCCCCGCCATCTG | |
| | CCACGACGGCAAGGCCCACTTCCCCAGGGAGGGCGTGTTCGTGAG | |
| | CAACGGCACCCACTGGTTCGTGACCCAGAGGAACTTCTACGAGCCC | |
| | CAGATCATCACCACCGACAACACCTTCGTGAGCGGCAACTGCGACG | |
| | TGGTGATCGGCATCGTGAACAACACCGTGTACGACCCCCTGCAGCC | |
| | CGAGCTGGACAGCTTCAAGGAGGAGCTGGACAAGTACTTCAAGAAC | |
| | CACACCAGCCCCGACGTGGACCTGGGCGACATCAGCGGCATCAACG | |
| | CCAGCGTGGTGAACATCCAGAAGGAGATCGACAGGCTGAACGAGGT | |
| | GGCCAAGAACCTGAACGAGAGCCTGATCGACCTGCAGGAGCTGGGC | |
| | AAGTACGAGCAGTACATCAAGTGGCCCTGGTACATCTGGCTGGGCTT | |
| | CATCGCCGGCCTGATCGCCATCGTGATGGTGACCATCATGCTGTGCT | |
| | GCATGACCAGCTGCTGCAGCTGCCTGAAGGGCTGCTGCAGCTGCGG | |
| | CAGCTGCTGCAAGTTCGACGAGGACGACAGCGAGCCCGTGCTGAAG | |
| | GGCGTGAAGCTGCACTACACCGGAAGCGGAGCCACGAACTTCTCTC | |
| | 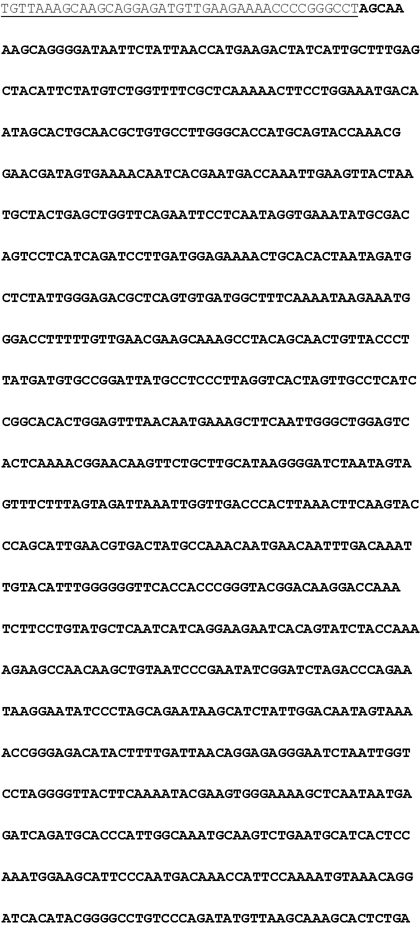 | |
| | 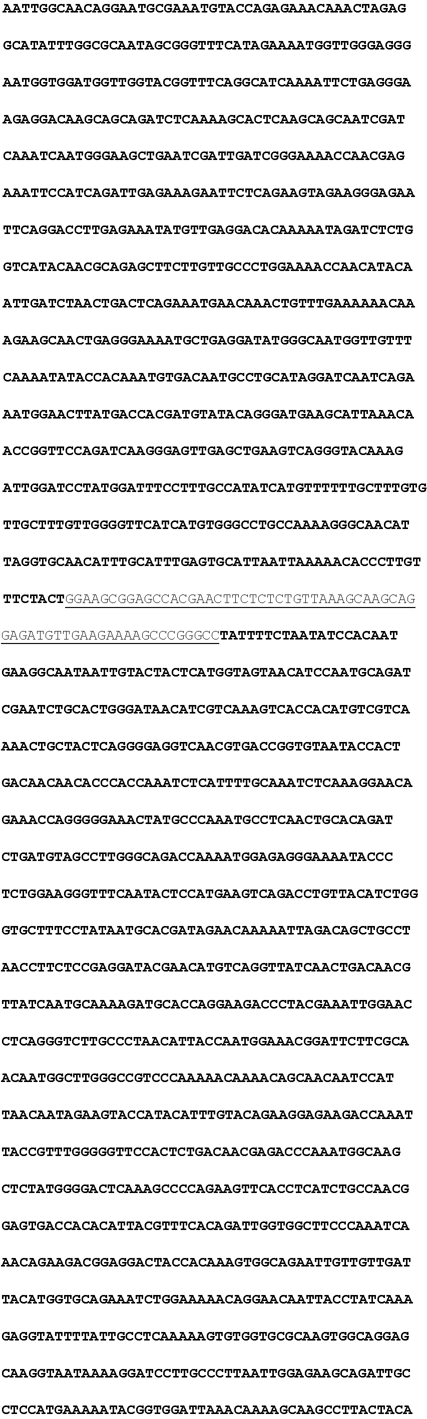 | |
| | 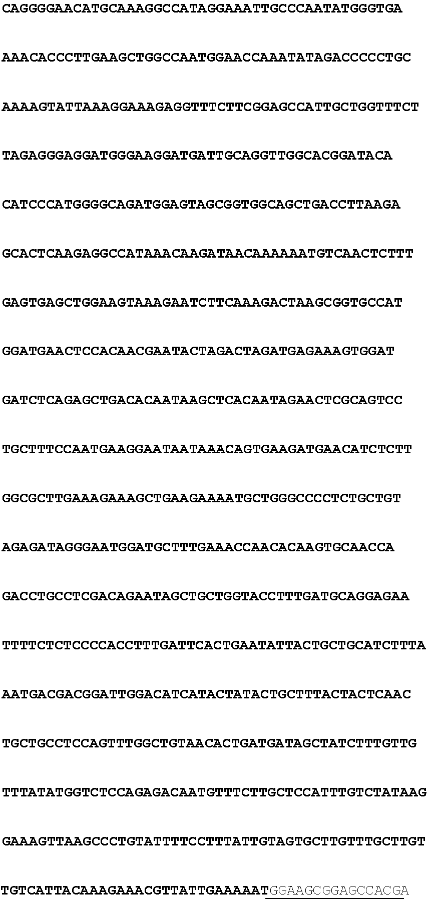 | |
| | ACTTCTCTCTGTTAAAGCAAGCAGGAGATGTTGAAGAAAACCCCGGG | |
| | CCTTTCGGAGCTATTGCTGCTTTCTTGGAAGGAGGATGGGAAGGAAT | |
| | GATTGCAGGTTGGCACGGATACACATCTCATGGAGCACATGGAGTAG | |
| | CAGTGGCAGCAGACCTTAAGAGTACCCATGATGACTCGAGCTGGTAC | |
| | TGCATGCACGCAATGCTAGCTGCCCCTTTCCCGTCCTGGGTACCCC | |
| | GAGTCTCCCCCGACCTCGGGTCCCAGGTATGCTCCCACCTCCACCT | |
| | GCCCCACTCACCACCTCTGCTAGTTCCAGACACCTCCCAAGCACGCA | |
| | GCAATGCAGCTCAAAACGCTTAGCCTAGCCACACCCCCACGGGAAA | |
| | CAGCAGTGATTAACCTTTAGCAATAAACGAAAGTTTAACTAAGCTATA | |
| | CTAACCCCAGGGTTGGTCAATTTCGTGCCAGCCACACCCTGGAGCTA | |
| | GCAAAAAAAA | |
| Pan-CoV-Flu | TAGTTATTAATAGTAATCAATTACGGGGTCATTAGTTCATAGCCCATAT | 396 |
| Vaccine #8 | ATGGAGTTCCGCGTTACATAACTTACGGTAAATGGCCCGCCTGGCTG | |
| CoV-Omicron | ACCGCCCAACGACCCCCGCCCATTGACGTCAATAATGACGTATGTTC | |
| BA.2 Spike(2 | CCATAGTAACGCCAATAGGGACTTTCCATTGACGTCAATGGGTGGAG | |
| proline | TATTTACGGTAAACTGCCCACTTGGCAGTACATCAAGTGTATCATATG | |
| substitution)- | CCAAGTACGCCCCCTATTGACGTCAATGACGGTAAATGGCCCGCCTG | |
| Influenza (HA) | GCATTATGCCCAGTACATGACCTTATGGGACTTTCCTACTTGGCAGTA | |
| | CATCTACGTATTAGTCATCGCTATTACCATGGTGATGCGGTTTTGGCA | |
| | GTACATCAATGGGCGTGGATAGCGGTTTGACTCACGGGGATTTCCAA | |
| | GTCTCCACCCCATTGACGTCAATGGGAGTTTGTTTTGGCACCAAAAT | |
| | CAACGGGACTTTCCAAAATGTCGTAACAACTCCGCCCCATTGACGCA | |
| | AATGGGCGGTAGGCGTGTACGGTGGGAGGTCTATATAAGCAGAGCT | |
| | GGTTTAGTGAACCGTCAGATCGAGAATAAACTAGTATTCTTCTGGTCC | |
| | CCACAGACTCAGAGAGAACCCGCCACCATGTTCGTGTTCCTGGTGCT | |
| | GCTGCCCCTGGTGAGCAGCCAGTGCGTGAACCTGATCACCAGGACC | |
| | CAGAGCTACACCAACAGCTTCACCAGGGGCGTGTACTACCCCGACA | |
| | AGGTGTTCAGGAGCAGCGTGCTGCACAGCACCCAGGACCTGTTCCT | |
| | GCCCTTCTTCAGCAACGTGACCTGGTTCCACGCCATCCACGTGAGC | |
| | GGCACCAACGGCACCAAGAGGTTCGACAACCCCGTGCTGCCCTTCA | |
| | ACGACGGCGTGTACTTCGCCAGCACCGAGAAGAGCAACATCATCAG | |
| | GGGCTGGATCTTCGGCACCACCCTGGACAGCAAGACCCAGAGCCTG | |
| | CTGATCGTGAACAACGCCACCAACGTGGTGATCAAGGTGTGCGAGTT | |
| | CCAGTTCTGCAACGACCCCTTCCTGGACGTGTACTACCACAAGAACA | |
| | ACAAGAGCTGGATGGAGAGCGAGTTCAGGGTGTACAGCAGCGCCAA | |
| | CAACTGCACCTTCGAGTACGTGAGCCAGCCCTTCCTGATGGACCTG | |
| | GAGGGCAAGCAGGGCAACTTCAAGAACCTGAGGGAGTTCGTGTTCA | |
| | AGAACATCGACGGCTACTTCAAGATCTACAGCAAGCACACCCCCATC | |
| | AACCTGGGCAGGGACCTGCCCCAGGGCTTCAGCGCCCTGGAGCCC | |
| | CTGGTGGACCTGCCCATCGGCATCAACATCACCAGGTTCCAGACCCT | |
| | GCTGGCCCTGCACAGGAGCTACCTGACCCCCGGCGACAGCAGCAG | |
| | CGGCTGGACCGCCGGCGCCGCCGCCTACTACGTGGGCTACCTGCA | |
| | GCCCAGGACCTTCCTGCTGAAGTACAACGAGAACGGCACCATCACC | |
| | GACGCCGTGGACTGCGCCCTGGACCCCCTGAGCGAGACCAAGTGC | |
| | ACCCTGAAGAGCTTCACCGTGGAGAAGGGCATCTACCAGACCAGCA | |
| | ACTTCAGGGTGCAGCCCACCGAGAGCATCGTGAGGTTCCCCAACAT | |
| | CACCAACCTGTGCCCCTTCGACGAGGTGTTCAACGCCACCAGGTTC | |
| | GCCAGCGTGTACGCCTGGAACAGGAAGAGGATCAGCAACTGCGTGG | |
| | CCGACTACAGCGTGCTGTACAACTTCGCCCCCTTCTTCGCCTTCAAG | |
| | TGCTACGGCGTGAGCCCCACCAAGCTGAACGACCTGTGCTTCACCA | |
| | ACGTGTACGCCGACAGCTTCGTGATCAGGGGCAACGAGGTGAGCCA | |
| | GATCGCCCCCGGCCAGACCGGCAACATCGCCGACTACAACTACAAG | |
| | CTGCCCGACGACTTCACCGGCTGCGTGATCGCCTGGAACAGCAACA | |
| | AGCTGGACAGCAAGGTGGGCGGCAACTACAACTACCTGTACAGGCT | |
| | GTTCAGGAAGAGCAACCTGAAGCCCTTCGAGAGGGACATCAGCACC | |
| | GAGATCTACCAGGCCGGCAACAAGCCCTGCAACGGCGTGGCCGGCT | |
| | TCAACTGCTACTTCCCCCTGAGGAGCTACGGCTTCAGGCCCACCTAC | |
| | GGCGTGGGCCACCAGCCCTACAGGGTGGTGGTGCTGAGCTTCGAG | |
| | CTGCTGCACGCCCCCGCCACCGTGTGCGGCCCCAAGAAGAGCACCA | |
| | ACCTGGTGAAGAACAAGTGCGTGAACTTCAACTTCAACGGCCTGACC | |
| | GGCACCGGCGTGCTGACCGAGAGCAACAAGAAGTTCCTGCCCTTCC | |
| | AGCAGTTCGGCAGGGACATCGCCGACACCACCGACGCCGTGAGGG | |
| | ACCCCCAGACCCTGGAGATCCTGGACATCACCCCCTGCAGCTTCGG | |
| | CGGCGTGAGCGTGATCACCCCCGGCACCAACACCAGCAACCAGGTG | |
| | GCCGTGCTGTACCAGGGCGTGAACTGCACCGAGGTGCCCGTGGCC | |
| | ATCCACGCCGACCAGCTGACCCCCACCTGGAGGGTGTACAGCACCG | |
| | GCAGCAACGTGTTCCAGACCAGGGCCGGCTGCCTGATCGGCGCCG | |
| | AGTACGTGAACAACAGCTACGAGTGCGACATCCCCATCGGCGCCGG | |
| | CATCTGCGCCAGCTACCAGACCCAGACCAAGAGCCACCAGCAGGCC | |
| | CAGAGCGTGGCCAGCCAGAGCATCATCGCCTACACCATGAGCCTGG | |
| | GCGCCGAGAACAGCGTGGCCTACAGCAACAACAGCATCGCCATCCC | |
| | CACCAACTTCACCATCAGCGTGACCACCGAGATCCTGCCCGTGAGC | |
| | ATGACCAAGACCAGCGTGGACTGCACCATGTACATCTGCGGCGACA | |
| | GCACCGAGTGCAGCAACCTGCTGCTGCAGTACGGCAGCTTCTGCAC | |
| | CCAGCTGAAGAGGGCCCTGACCGGCATCGCCGTGGAGCAGGACAA | |
| | GAACACCCAGGAGGTGTTCGCCCAGGTGAAGCAGATCTACAAGACC | |
| | CCCCCCATCAAGTACTTCGGCGGCTTCAACTTCAGCCAGATCCTGCC | |
| | CGACCCCAGCAAGCCCAGCAAGAGGAGCTTCATCGAGGACCTGCTG | |
| | TTCAACAAGGTGACCCTGGCCGACGCCGGCTTCATCAAGCAGTACG | |
| | GCGACTGCCTGGGCGACATCGCCGCCAGGGACCTGATCTGCGCCC | |
| | AGAAGTTCAACGGCCTGACCGTGCTGCCCCCCCTGCTGACCGACGA | |
| | GATGATCGCCCAGTACACCAGCGCCCTGCTGGCCGGCACCATCACC | |
| | AGCGGCTGGACCTTCGGCGCCGGCGCCGCCCTGCAGATCCCCTTC | |
| | GCCATGCAGATGGCCTACAGGTTCAACGGCATCGGCGTGACCCAGA | |
| | ACGTGCTGTACGAGAACCAGAAGCTGATCGCCAACCAGTTCAACAGC | |
| | GCCATCGGCAAGATCCAGGACAGCCTGAGCAGCACCGCCAGCGCC | |
| | CTGGGCAAGCTGCAGGACGTGGTGAACCACAACGCCCAGGCCCTGA | |
| | ACACCCTGGTGAAGCAGCTGAGCAGCAAGTTCGGCGCCATCAGCAG | |
| | CGTGCTGAACGACATCCTGAGCAGGCTGGACCCCCCCGAGGCCGA | |
| | GGTGCAGATCGACAGGCTGATCACCGGCAGGCTGCAGAGCCTGCAG | |
| | ACCTACGTGACCCAGCAGCTGATCAGGGCCGCCGAGATCAGGGCCA | |
| | GCGCCAACCTGGCCGCCACCAAGATGAGCGAGTGCGTGCTGGGCC | |
| | AGAGCAAGAGGGTGGACTTCTGCGGCAAGGGCTACCACCTGATGAG | |
| | CTTCCCCCAGAGCGCCCCCCACGGCGTGGTGTTCCTGCACGTGACC | |
| | TACGTGCCCGCCCAGGAGAAGAACTTCACCACCGCCCCCGCCATCT | |
| | GCCACGACGGCAAGGCCCACTTCCCCAGGGAGGGCGTGTTCGTGA | |
| | GCAACGGCACCCACTGGTTCGTGACCCAGAGGAACTTCTACGAGCC | |
| | CCAGATCATCACCACCGACAACACCTTCGTGAGCGGCAACTGCGAC | |
| | GTGGTGATCGGCATCGTGAACAACACCGTGTACGACCCCCTGCAGC | |
| | CCGAGCTGGACAGCTTCAAGGAGGAGCTGGACAAGTACTTCAAGAA | |
| | CCACACCAGCCCCGACGTGGACCTGGGCGACATCAGCGGCATCAAC | |
| | GCCAGCGTGGTGAACATCCAGAAGGAGATCGACAGGCTGAACGAGG | |
| | TGGCCAAGAACCTGAACGAGAGCCTGATCGACCTGCAGGAGCTGGG | |
| | CAAGTACGAGCAGTACATCAAGTGGCCCTGGTACATCTGGCTGGGC | |
| | TTCATCGCCGGCCTGATCGCCATCGTGATGGTGACCATCATGCTGTG | |
| | CTGCATGACCAGCTGCTGCAGCTGCCTGAAGGGCTGCTGCAGCTGC | |
| | GGCAGCTGCTGCAAGTTCGACGAGGACGACAGCGAGCCCGTGCTGA | |
| | AGGGCGTGAAGCTGCACTACACCGGAAGCGGAGCCACGAACTTCTC | |
| | TCTGTTAAAGCAAGCAGGAGATGTTGAAGAAAACCCCGGGCCTATGA | |
| | AGGCAATACTAGTAGTTCTGCTATATACATTTGCAACCGCAAATGCAG | |
| | ACACATTATGTATAGGTTATCATGCGAACAACTCAACAGACACTGTAG | |
| | ACACAGTACTAGAAAAGAATGTAACAGTAACACACTCTGTTAACCTTC | |
| | TAGAAGACAAGCATAACGGGAAACTATGCAAACTAAGAGGGGTAGCC | |
| | CCATTGCATTTGGGTAAATGTAACATTGCTGGCTGGATCCTGGGAAA | |
| | TCCAGAGTGTGAATCACTCTCCACAGCAAGCTCATGGTCCTACATTG | |
| | TGGAAACATCTAGTTCAGACAATGGAACGTGTTACCCAGGAGATTTC | |
| | ATCGATTATGAGGAGCTAAGAGAGCAATTGAGCTCAGTGTCATCATTT | |
| | GAAAGGTTTGAGATATTCCCCAAGACAAGTTCATGGCCCAATCATGA | |
| | CTCGAACAAAGGTGTAACGACAGCATGTCCTCATGCTGGAGCAAAAA | |
| | GCTTCTACAAAAATTTAATATGGCTAGTTAAAAAAGGAAATTCATACC | |
| | CAAAGCTCAGCAAATCCTACATTAATGATAAAGGGAAAGAAGTCCTC | |
| | GTGCTATGGGGCATTCACCATCCACCTACTAGTGCTGACCAACAAAG | |
| | TCTCTATCAGAATGCAGATGCATATGTTTTTGTGGGGACATCAAGATA | |
| | CAGCAAGAAGTTCAAGCCGGAAATAGCAATAAGGCCCAAAGTGAGG | |
| | GATCAAGAAGGGAGAATGAACTATTACTGGACACTAGTAGAGCCGGG | |
| | AGACAAAATAACATTCGAAGCAACTGGAAATCTAGTGGTACCGAGAT | |
| | ATGCATTCGCAATGGAAAGAAATGCTGGATCTGGTATTATCATTTCAG | |
| | ATACACCAGTCCACGATTGCAATACAACTTGTCAGACACCCAAGGGT | |
| | GCTATAAACACCAGCCTCCCATTTCAGAACATACATCCGATCACAATT | |
| | GGAAAATGTCCAAAATATGTAAAAAGCACAAAATTGAGACTGGCCAC | |
| | AGGATTGAGGAATGTCCCGTCCATTCAATCTAGAGGCCTATTTGGGG | |
| | CCATTGCCGGTTTCATTGAAGGGGGGTGGACAGGGATGGTAGATGG | |
| | ATGGTACGGTTATCACCATCAAAATGAGCAGGGGTCAGGATATGCAG | |
| | CCGACCTGAAGAGCACACAGAATGCCATTGACGAGATTACTAACAAA | |
| | GTAAACTCTGTTATTGAAAAAATGAATACACAGTTCACAGCAGTAGGT | |
| | AAAGAGTTCAACCACCTGGAAAAAAGAATAGAGAATTTAAATAAAAAA | |
| | GTTGATGATGGTTTCCTGGACATTTGGACTTACAATGCCGAACTGTTG | |
| | GTTCTATTGGAAAATGAAAGAACTTTGGACTACCACGATTCAAAGGTG | |
| | AAGAACTTATATGAAAAGGTAAGAAGCCAGTTAAAAAACAATGCCAAG | |
| | GAAATTGGAAACGGCTGCTTTGAATTTTACCACAAATGTGATAACACG | |
| | TGCATGGAAAGTGTCAAAAATGGGACTTATGACTACCCAAAATACTCA | |
| | GAGGAAGCAAAATTAAACAGAGAAGAAATAGATGGGGTAAAGCTGGA | |
| | ATCAACAAGGATTTACCAGATTTTGGCGATCTATTCAACCGTCGCCAG | |
| | TTCATTGGTACTGGTAGTCTCCCTGGGGGCAATCAGTTTCTGGATGT | |
| | GCTCTAATGGGTCTCTACAGTGTAGAATATGTATTTAAGGAAGCGGA | |
| | GCCACGAACTTCTCTCTGTTAAAGCAAGCAGGAGATGTTGAAGAAAA | |
| | CCCCGGGCCTAGCAAAAGCAGGGGATAATTCTATTAACCATGAAGAC | |
| | TATCATTGCTTTGAGCTACATTCTATGTCTGGTTTTCGCTCAAAAACTT | |
| | CCTGGAAATGACAATAGCACTGCAACGCTGTGCCTTGGGCACCATGC | |
| | AGTACCAAACGGAACGATAGTGAAAACAATCACGAATGACCAAATTG | |
| | AAGTTACTAATGCTACTGAGCTGGTTCAGAATTCCTCAATAGGTGAAA | |
| | TATGCGACAGTCCTCATCAGATCCTTGATGGAGAAAACTGCACACTA | |
| | ATAGATGCTCTATTGGGAGACCCTCAGTGTGATGGCTTTCAAAATAA | |
| | GAAATGGGACCTTTTTGTTGAAGAAGCAAAGCCTACAGCAACTGTT | |
| | ACCCTTATGATGTGCCGGATTATGCCTCCCTTAGGTCACTAGTTGCC | |
| | TCATCCGGCACACTGGAGTTTAACAATGAAAGCTTCAATTGGGCTGG | |
| | AGTCACTCAAAACGGAACAAGTTCTGCTTGCATAAGGGGATCTAATA | |
| | GTAGTTTCTTTAGTAGATTAAATTGGTTGACCCACTTAAACTTCAAGTA | |
| | CCCAGCATTGAACGTGACTATGCCAAACAATGAACAATTTGACAAATT | |
| | GTACATTTGGGGGGTTCACCACCCGGGTACGGACAAGGACCAAATC | |
| | TTCCTGTATGCTCAATCATCAGGAAGAATCACAGTATCTACCAAAAGA | |
| | AGCCAACAAGCTGTAATCCCGAATATCGGATCTAGACCCAGAATAAG | |
| | GAATATCCCTAGCAGAATAAGCATCTATTGGACAATAGTAAAACCGG | |
| | GAGACATACTTTTGATTAACAGCACAGGGAATCTAATTGCTCCTAGG | |
| | GGTTACTTCAAAATACGAAGTGGGAAAAGCTCAATAATGAGATCAGAT | |
| | GCACCCATTGGCAAATGCAAGTCTGAATGCATCACTCCAAATGGAAG | |
| | CATTCCCAATGACAAACCATTCCAAAATGTAAACAGGATCACATACGG | |
| | GGCCTGTCCCAGATATGTTAAGCAAAGCACTCTGAAATTGGCAACAG | |
| | GAATGCGAAATGTACCAGAGAAACAAACTAGAGGCATATTTGGCGCA | |
| | ATAGCGGGTTTCATAGAAAATGGTTGGGAGGGAATGGTGGATGGTTG | |
| | GTACGGTTTCAGGCATCAAAATTCTGAGGGAAGAGGACAAGCAGCA | |
| | GATCTCAAAAGCACTCAAGCAGCAATCGATCAAATCAATGGGAAGCT | |
| | GAATCGATTGATCGGGAAAACCAACGAGAAATTCCATCAGATTGAGA | |
| | AAGAATTCTCAGAAGTAGAAGGGAGAATTCAGGACCTTGAGAAATAT | |
| | GTTGAGGACACAAAAATAGATCTCTGGTCATACAACGCAGAGCTTCT | |
| | TGTTGCCCTGGAAAACCAACATACAATTGATCTAACTGACTCAGAAAT | |
| | GAACAAACTGTTTGAAAAAACAAAGAAGCAACTGAGGGAAAATGCTG | |
| | AGGATATGGGCAATGGTTGTTTCAAAATATACCACAAATGTGACAATG | |
| | CCTGCATAGGATCAATCAGAAATGGAACTTATGACCACGATGTATACA | |
| | GGGATGAAGCATTAAACAACCGGTTCCAGATCAAGGGAGTTGAGCTG | |
| | AAGTCAGGGTACAAAGATTGGATCCTATGGATTTCCTTTGCCATATCA | |
| | TGTTTTTTGCTTTGTGTTGCTTTGTTGGGGTTCATCATGTGGGCCTGC | |
| | CAAAAGGGCAACATTAGGTGCAACATTTGCATTTGAGTGCATTAATTA | |
| | AAAACACCCTTGTTTCTACTGGAAGCGGAGCCACGAACTTCTCTCTG | |
| | TTAAAGCAAGCAGGAGATGTTGAAGAAAACCCCGGGCCTATTTTCTA | |
| | ATATCCACAAAATGAAGGCAATAATTGTACTACTCATGGTAGTAACAT | |
| | CCAATGCAGATCGAATCTGCACTGGGATAACATCGTCAAACTCACCA | |
| | CATGTCGTCAAAACTGCTACTCAAGGGGAGGTCAACGTGACCGGTGT | |
| | AATACCACTGACAACAACACCCACCAAATCTCATTTTGCAAATCTCAA | |
| | AGGAACAGAAACCAGGGGGAAACTATGCCCAAAATGCCTCAACTGCA | |
| | CAGATCTGGATGTAGCCTTGGGCAGACCAAAATGCACAGGGAAAATA | |
| | CCCTCTGCAAGGGTTTCAATACTCCATGAAGTCAGACCTGTTACATCT | |
| | GGGTGCTTTCCTATAATGCACGATAGAACAAAAATTAGACAGCTGCC | |
| | TAACCTTCTCCGAGGATACGAACATGTCAGGTTATCAACTCACAACGT | |
| | TATCAATGCAAAAGATGCACCAGGAAGACCCTACGAAATTGGAACCT | |
| | CAGGGTCTTGCCCTAACATTACCAATGGAAACGGATTCTTCGCAACA | |
| | ATGGCTTGGGCCGTCCCAAAAAACAAAACAGCAACAAATCCATTAAC | |
| | AATAGAAGTACCATACATTTGTACAGAAGGAGAAGACCAAATTACCGT | |
| | TTGGGGGTTCCACTCTGACAACGAGACCCAAATGGCAAAGCTCTATG | |
| | GGGACTCAAAGCCCCAGAAGTTCACCTCATCTGCCAACGGAGTGAC | |
| | CACACATTACGTTTCACAGATTGGTGGCTTCCCAAATCAAACAGAAGA | |
| | CGGAGGACTACCACAAAGTGGCAGAATTGTTGTTGATTACATGGTGC | |
| | AGAAATCTGGAAAAACAGGAACAATTACCTATCAAAGAGGTATTTTAT | |
| | TGCCTCAAAAAGTGTGGTGCGCAAGTGGCAGGAGCAAGGTAATAAAA | |
| | GGATCCTTGCCCTTAATTGGAGAAGCAGATTGCCTCCATGAAAAATA | |
| | CGGTGGATTAAACAAAAGCAAGCCTTACTACACAGGGGAACATGCAA | |
| | AGGCCATAGGAAATTGCCCAATATGGGTGAAAACACCCTTGAAGCTG | |
| | GCCAATGGAACCAAATATAGACCCCCTGCAAAACTATTAAAGGAAAG | |
| | AGGTTTCTTCGGAGCCATTGCTGGTTTCTTAGAGGGAGGATGGGAAG | |
| | GAATGATTGCAGGTTGGCACGGATACACATCCCATGGGGCACATGG | |
| | AGTAGCGGTGGCAGCTGACCTTAAGAGCACTCAAGAGGCCATAAAC | |
| | AAGATAACAAAAAATCTCAACTCTTTGAGTGAGCTGGAAGTAAAGAAT | |
| | CTTCAAAGACTAAGCGGTGCCATGGATGAACTCCACAACGAAATACT | |
| | AGAACTAGATGAGAAAGTGGATGATCTCAGAGCTGACACAATAAGCT | |
| | CACAAATAGAACTCGCAGTCCTGCTTTCCAATGAAGGAATAATAAACA | |
| | GTGAAGATGAACATCTCTTGGCGCTTGAAAGAAAGCTGAAGAAAATG | |
| | CTGGGCCCCTCTGCTGTAGAGATAGGGAATGGATGCTTTGAAACCAA | |
| | ACACAAGTGCAACCAGACCTGCCTCGACAGAATAGCTGCTGGTACCT | |
| | TTGATGCAGGAGAATTTTCTCTCCCCACCTTTGATTCACTGAATATTA | |
| | CTGCTGCATCTTTAAATGACGACGGATTGGACAATCATACTATACTGC | |
| | TTTACTACTCAACTGCTGCCTCCAGTTTGGCTGTAACACTGATGATAG | |
| | CTATCTTTGTTGTTTATATGGTCTCCAGAGACAATGTTTCTTGCTCCAT | |
| | TTGTCTATAAGGAAAGTTAAGCCCTGTATTTTCCTTTATTGTAGTGCTT | |
| | GTTTGCTTGTTGTCATTACAAAGAAACGTTATTGAAAAATGGAAGCGG | |
| | AGCCACGAACTTCTCTCTGTTAAAGCAAGCAGGAGATGTTGAAGAAA | |
| | ACCCCGGGCCTTTCGGAGCTATTGCTGGTTTCTTGGAAGGAGGATG | |
| | GGAAGGAATGATTGCAGGTTGGCACGGATACACATCTCATGGAGCA | |
| | CATGGAGTAGCAGTGGCAGCAGACCTTAAGAGTACCCATGATGACTC | |
| | GAGCTGGTACTGCATGCACGCAATGCTAGCTGCCCCTTTCCCGTCCT | |
| | GGGTACCCCGAGTCTCCCCCGACCTCGGGTCCCAGGTATGCTCCCA | |
| | CCTCCACCTGCCCCACTCACCACCTCTGCTAGTTCCAGACACCTCCC | |
| | AAGCACGCAGCAATGCAGCTCAAAACGCTTAGCCTAGCCACACCCC | |
| | CACGGGAAACAGCAGTGATTAACCTTTAGCAATAAACGAAAGTTTAAC | |
| | TAAGCTATACTAACCCCAGGGTTGGTCAATTTCGTGCCAGCCACACC | |
| | CTGGAGCTAGCAAAAAAAA | |
| Pan-CoV-Flu | CTCGACATTGATTATTGACTAGTTATTAATAGTAATCAATTACGGGGT | 397 |
| Vaccine #9 | CATTAGTTCATAGCCCATATATGGAGTTCCGCGTTACATAACTTACGG | |
| CoV- Omicron | TAAATGGCCCGCCTGGCTGACCGCCCAACGACCCCCGCCCATTGAC | |
| BA.2 | GTCAATAATGACGTATGTTCCCATAGTAACGCCAATAGGGACTTTCCA | |
| Spike(HexaPro)- | TTGACGTCAATGGGTGGAGTATTTACGGTAAACTGCCCACTTGGCAG | |
| Influenza (HA) | TACATCAAGTGTATCATATGCCAAGTACGCCCCCTATTGACGTCAATG | |
| | ACGGTAAATGGCCCGCCTGGCATTATGCCCAGTACATGACCTTATGG | |
| | GACTTTCCTACTTGGCAGTACATCTACGTATTAGTCATCGCTATTACC | |
| | ATGGTCGAGGTGAGCCCCACGTTCTGCTTCACTCTCCCCATCTCCCC | |
| | CCCCTCCCCACCCCCAATTTTGTATTTATTTATTTTTTAATTATTTTGT | |
| | GCAGCGATGGGGGCGGGGGGGGGGGGGGGGCGCGCGCCAGGCG | |
| | GGGCGGGGCGGGGCGAGGGGCGGGGCGGGGCGAGGCGGAGAGG | |
| | TGCGGCGGCAGCCAATCAGAGCGGCGCGCTCCGAAAGTTTCCTTTT | |
| | ATGGCGAGGCGGCGGCGGCGGCGGCCCTATAAAAAGCGAAGCGCG | |
| | CGGCGGGCGGGAGTCGCTGCGCGCTGCCTTCGCCCCGTGCCCCGC | |
| | TCCGCCGCCGCCTCGCGCCGCCCGCCCCGGCTCTGACTGACCGCG | |
| | TTACTCCCACAGGTGAGCGGGCGGGACGGCCCTTCTCCTCCGGGCT | |
| | GTAATTAGCGCTTGGTTTAATGACGGCTTGTTTCTTTTCTGTGGCTGC | |
| | GTGAAAGCCTTGAGGGGCTCCGGGAGGGCCCTTTGTGCGGGGGGA | |
| | GCGGCTCGGGGGGTGCGTGCGTGTGTGTGTGCGTGGGGAGCGCCG | |
| | CGTGCGGCTCCGCGCTGCCCGGCGGCTGTGAGCGCTGCGGGCGCG | |
| | GCGCGGGGCTTTGTGCGCTCCGCAGTGTGCGCGAGGGGAGCGCGG | |
| | CCGGGGGCGGTGCCCCGCGGTGCGGGGGGGGCTGCGAGGGGAAC | |
| | AAAGGCTGCGTGCGGGGTGTGTGCGTGGGGGGGTGAGCAGGGGGT | |
| | GTGGGCGCGTCGGTCGGGCTGCAACCCCCCCTGCACCCCCCTCCC | |
| | CGAGTTGCTGAGCACGGCCCGGCTTCGGGTGCGGGGCTCCGTACG | |
| | GGGCGTGGCGCGGGGCTCGCCGTGCCGGGCGGGGGGTGGCGGCA | |
| | GGTGGGGGTGCCGGGCGGGGCGGGGCCGCCTCGGGCCGGGGAG | |
| | GGCTCGGGGGAGGGGCGCGGCGGCCCCCGGAGCGCCGGCGGCTG | |
| | TCGAGGCGCGGCGAGCCGCAGCCATTGCCTTTTATGGTAATCGTGC | |
| | GAGAGGGCGCAGGGACTTCCTTTGTCCCAAATCTGTGCGGAGCCGA | |
| | AATCTGGGAGGCGCCGCCGCACCCCCTCTAGCGGGCGCGGGGCGA | |
| | AGCGGTGCGGCGCCGGCAGGAAGGAAATGGGCGGGGAGGGCCTTC | |
| | GTGCGTCGCCGCGCCGCCGTCCCCTTCTCCCTCTCCAGCCTCGGGG | |
| | CTGTCCGCGGGGGGACGGCTGCCTTCGGGGGGGACGGGGCAGGG | |
| | CGGGGTTCGGCTTCTGGCGTGTGACCGGCGGCTCTAGAGCCTCTGC | |
| | TAACCATGTTCATGCCTTCTTCTTTTTCCTACAGCTCCTGGGCAACGT | |
| | GCTGGTTATTGTGCTGTCTCATCATTTTGGCAAAGAATTGGAGAATAA | |
| | ACTAGTATTCTTCTGGTCCCCACAGACTCAGAGAGAACCCGCCACCA | |
| | TGTTCGTGTTCCTGGTGCTGCTGCCCCTGGTGAGCAGCCAGTGCGT | |
| | GAACCTGATCACCAGGACCCAGAGCTACACCAACAGCTTCACCAGG | |
| | GGCGTGTACTACCCCGACAAGGTGTTCAGGAGCAGCGTGCTGCACA | |
| | GCACCCAGGACCTGTTCCTGCCCTTCTTCAGCAACGTGACCTGGTTC | |
| | CACGCCATCCACGTGAGCGGCACCAACGGCACCAAGAGGTTCGACA | |
| | ACCCCGTGCTGCCCTTCAACGACGGCGTGTACTTCGCCAGCACCGA | |
| | GAAGAGCAACATCATCAGGGGCTGGATCTTCGGCACCACCCTGGAC | |
| | AGCAAGACCCAGAGCCTGCTGATCGTGAACAACGCCACCAACGTGG | |
| | TGATCAAGGTGTGCGAGTTCCAGTTCTGCAACGACCCCTTCCTGGAC | |
| | GTGTACTACCACAAGAACAACAAGAGCTGGATGGAGAGCGAGTTCA | |
| | GGGTGTACAGCAGCGCCAACAACTGCACCTTCGAGTACGTGAGCCA | |
| | GCCCTTCCTGATGGACCTGGAGGGCAAGCAGGGCAACTTCAAGAAC | |
| | CTGAGGGAGTTCGTGTTCAAGAACATCGACGGCTACTTCAAGATCTA | |
| | CAGCAAGCACACCCCCATCAACCTGGGCAGGGACCTGCCCCAGGGC | |
| | TTCAGCGCCCTGGAGCCCCTGGTGGACCTGCCCATCGGCATCAACA | |
| | TCACCAGGTTCCAGACCCTGCTGGCCCTGCACAGGAGCTACCTGAC | |
| | CCCCGGCGACAGCAGCAGCGGCTGGACCGCCGGCGCCGCCGCCTA | |
| | CTACGTGGGCTACCTGCAGCCCAGGACCTTCCTGCTGAAGTACAAC | |
| | GAGAACGGCACCATCACCGACGCCGTGGACTGCGCCCTGGACCCC | |
| | CTGAGCGAGACCAAGTGCACCCTGAAGAGCTTCACCGTGGAGAAGG | |
| | GCATCTACCAGACCAGCAACTTCAGGGTGCAGCCCACCGAGAGCAT | |
| | CGTGAGGTTCCCCAACATCACCAACCTGTGCCCCTTCGACGAGGTGT | |
| | TCAACGCCACCAGGTTCGCCAGCGTGTACGCCTGGAACAGGAAGAG | |
| | GATCAGCAACTGCGTGGCCGACTACAGCGTGCTGTACAACTTCGCC | |
| | CCCTTCTTCGCCTTCAAGTGCTACGGCGTGAGCCCCACCAAGCTGAA | |
| | CGACCTGTGCTTCACCAACGTGTACGCCGACAGCTTCGTGATCAGG | |
| | GGCAACGAGGTGAGCCAGATCGCCCCCGGCCAGACCGGCAACATC | |
| | GCCGACTACAACTACAAGCTGCCCGACGACTTCACCGGCTGCGTGA | |
| | TCGCCTGGAACAGCAACAAGCTGGACAGCAAGGTGGGCGGCAACTA | |
| | CAACTACCTGTACAGGCTGTTCAGGAAGAGCAACCTGAAGCCCTTCG | |
| | AGAGGGACATCAGCACCGAGATCTACCAGGCCGGCAACAAGCCCTG | |
| | CAACGGCGTGGCCGGCTTCAACTGCTACTTCCCCCTGAGGAGCTAC | |
| | GGCTTCAGGCCCACCTACGGCGTGGGCCACCAGCCCTACAGGGTG | |
| | GTGGTGCTGAGCTTCGAGCTGCTGCACGCCCCCGCCACCGTGTGCG | |
| | GCCCCAAGAAGAGCACCAACCTGGTGAAGAACAAGTGCGTGAACTT | |
| | CAACTTCAACGGCCTGACCGGCACCGGCGTGCTGACCGAGAGCAAC | |
| | AAGAAGTTCCTGCCCTTCCAGCAGTTCGGCAGGGACATCGCCGACA | |
| | CCACCGACGCCGTGAGGGACCCCCAGACCCTGGAGATCCTGGACAT | |
| | CACCCCCTGCAGCTTCGGCGGCGTGAGCGTGATCACCCCCGGCACC | |
| | AACACCAGCAACCAGGTGGCCGTGCTGTACCAGGGCGTGAACTGCA | |
| | CCGAGGTGCCCGTGGCCATCCACGCCGACCAGCTGACCCCCACCTG | |
| | GAGGGTGTACAGCACCGGCAGCAACGTGTTCCAGACCAGGGCCGG | |
| | CTGCCTGATCGGCGCCGAGTACGTGAACAACAGCTACGAGTGCGAC | |
| | ATCCCCATCGGCGCCGGCATCTGCGCCAGCTACCAGACCCAGACCA | |
| | AGAGCCACCAGCAGGCCCAGAGCGTGGCCAGCCAGAGCATCATCG | |
| | CCTACACCATGAGCCTGGGCGCCGAGAACAGCGTGGCCTACAGCAA | |
| | CAACAGCATCGCCATCCCCACCAACTTCACCATCAGCGTGACCACCG | |
| | AGATCCTGCCCGTGAGCATGACCAAGACCAGCGTGGACTGCACCAT | |
| | GTACATCTGCGGCGACAGCACCGAGTGCAGCAACCTGCTGCTGCAG | |
| | TACGGCAGCTTCTGCACCCAGCTGAAGAGGGCCCTGACCGGCATCG | |
| | CCGTGGAGCAGGACAAGAACACCCAGGAGGTGTTCGCCCAGGTGAA | |
| | GCAGATCTACAAGACCCCCCCCATCAAGTACTTCGGCGGCTTCAACT | |
| | TCAGCCAGATCCTGCCCGACCCCAGCAAGCCCAGCAAGAGGAGCCC | |
| | CATCGAGGACCTGCTGTTCAACAAGGTGACCCTGGCCGACGCCGGC | |
| | TTCATCAAGCAGTACGGCGACTGCCTGGGCGACATCGCCGCCAGGG | |
| | ACCTGATCTGCGCCCAGAAGTTCAACGGCCTGACCGTGCTGCCCCC | |
| | CCTGCTGACCGACGAGATGATCGCCCAGTACACCAGCGCCCTGCTG | |
| | GCCGGCACCATCACCAGCGGCTGGACCTTCGGCGCCGGCCCCGCC | |
| | CTGCAGATCCCCTTCCCCATGCAGATGGCCTACAGGTTCAACGGCAT | |
| | CGGCGTGACCCAGAACGTGCTGTACGAGAACCAGAAGCTGATCGCC | |
| | AACCAGTTCAACAGCGCCATCGGCAAGATCCAGGACAGCCTGAGCA | |
| | GCACCCCCAGCGCCCTGGGCAAGCTGCAGGACGTGGTGAACCACAA | |
| | CGCCCAGGCCCTGAACACCCTGGTGAAGCAGCTGAGCAGCAAGTTC | |
| | GGCGCCATCAGCAGCGTGCTGAACGACATCCTGAGCAGGCTGGACC | |
| | CCCCCGAGGCCGAGGTGCAGATCGACAGGCTGATCACCGGCAGGC | |
| | TGCAGAGCCTGCAGACCTACGTGACCCAGCAGCTGATCAGGGCCGC | |
| | CGAGATCAGGGCCAGCGCCAACCTGGCCGCCACCAAGATGAGCGA | |
| | GTGCGTGCTGGGCCAGAGCAAGAGGGTGGACTTCTGCGGCAAGGG | |
| | CTACCACCTGATGAGCTTCCCCCAGAGCGCCCCCCACGGCGTGGTG | |
| | TTCCTGCACGTGACCTACGTGCCCGCCCAGGAGAAGAACTTCACCA | |
| | CCGCCCCCGCCATCTGCCACGACGGCAAGGCCCACTTCCCCAGGGA | |
| | GGGCGTGTTCGTGAGCAACGGCACCCACTGGTTCGTGACCCAGAGG | |
| | AACTTCTACGAGCCCCAGATCATCACCACCGACAACACCTTCGTGAG | |
| | CGGCAACTGCGACGTGGTGATCGGCATCGTGAACAACACCGTGTAC | |
| | GACCCCCTGCAGCCCGAGCTGGACAGCTTCAAGGAGGAGCTGGACA | |
| | AGTACTTCAAGAACCACACCAGCCCCGACGTGGACCTGGGCGACAT | |
| | CAGCGGCATCAACGCCAGCGTGGTGAACATCCAGAAGGAGATCGAC | |
| | AGGCTGAACGAGGTGGCCAAGAACCTGAACGAGAGCCTGATCGACC | |
| | TGCAGGAGCTGGGCAAGTACGAGCAGTACATCAAGTGGCCCTGGTA | |
| | CATCTGGCTGGGCTTCATCGCCGGCCTGATCGCCATCGTGATGGTG | |
| | ACCATCATGCTGTGCTGCATGACCAGCTGCTGCAGCTGCCTGAAGG | |
| | GCTGCTGCAGCTGCGGCAGCTGCTGCAAGTTCGACGAGGACGACAG | |
| | CGAGCCCGTGCTGAAGGGCGTGAAGCTGCACTACACCGGAAGCGG | |
| | AGCCACGAACTTCTCTCTGTTAAAGCAAGCAGGAGATGTTGAAGAAA | |
| | ACCCCGGGCCTATGAAGGCAATACTAGTAGTTCTGCTATATACATTTG | |
| | CAACCGCAAATGCAGACACATTATGTATAGGTTATCATGCGAACAACT | |
| | CAACAGACACTGTAGACACAGTACTAGAAAAGAATGTAACAGTAACA | |
| | CACTCTGTTAACCTTCTAGAAGACAAGCATAACGGGAAACTATGCAAA | |
| | CTAAGAGGGGTAGCCCCATTGCATTTGGGTAAATGTAACATTGCTGG | |
| | CTGGATCCTGGGAAATCCAGAGTGTGAATCACTCTCCACAGCAAGCT | |
| | CATGGTCCTACATTGTGGAAACATCTAGTTCAGACAATGGAACGTGTT | |
| | ACCCAGGAGATTTCATCGATTATGAGGAGCTAAGAGAGCAATTGAGC | |
| | TCAGTGTCATCATTTGAAAGGTTTGAGATATTCCCCAAGACAAGTTCA | |
| | TGGCCCAATCATGACTCGAACAAAGGTGTAACGACAGCATGTCCTCA | |
| | TGCTGGAGCAAAAAGCTTCTACAAAAATTTAATATGGCTAGTTAAAAA | |
| | AGGAAATTCATACCCAAAGCTCAGCAAATCCTACATTAATGATAAAGG | |
| | GAAAGAAGTCCTCGTGCTATGGGGCATTCACCATCCACCTACTAGTG | |
| | CTGACCAACAAAGTCTCTATCAGAATGCAGATGCATATGTTTTTGTGG | |
| | GGACATCAAGATACAGCAAGAAGTTCAAGCCGGAAATAGCAATAAGG | |
| | CCCAAAGTGAGGGATCAAGAAGGGAGAATGAACTATTACTGGACACT | |
| | AGTAGAGCCGGGAGACAAAATAACATTCGAAGCAACTGGAAATCTAG | |
| | TGGTACCGAGATATGCATTCGCAATGGAAAGAAATGCTGGATCTGGT | |
| | ATTATCATTTCAGATACACCAGTCCACGATTGCAATACAACTTGTCAG | |
| | ACACCCAAGGGTGCTATAAACACCAGCCTCCCATTTCAGAACATACA | |
| | TCCGATCACAATTGGAAAATGTCCAAAATATGTAAAAAGCACAAAATT | |
| | GAGACTGGCCACAGGATTGAGGAATGTCCCGTCCATTCAATCTAGAG | |
| | GCCTATTTGGGGCCATTGCCGGTTTCATTGAAGGGGGGTGGACAGG | |
| | GATGGTAGATGGATGGTACGGTTATCACCATCAAAATGAGCAGGGGT | |
| | CAGGATATGCAGCCGACCTGAAGAGCACACAGAATGCCATTGACGA | |
| | GATTACTAACAAAGTAAACTCTGTTATTGAAAAAATGAATACACAGTTC | |
| | ACAGCAGTAGGTAAAGAGTTCAACCACCTGGAAAAAAGAATAGAGAA | |
| | TTTAAATAAAAAAGTTGATGATGGTTTCCTGGACATTTGGACTTACAAT | |
| | GCCGAACTGTTGGTTCTATTGGAAAATGAAAGAACTTTGGACTACCA | |
| | CGATTCAAAGGTGAAGAACTTATATGAAAAGGTAAGAAGCCAGTTAAA | |
| | AAACAATGCCAAGGAAATTGGAAACGGCTGCTTTGAATTTTACCACAA | |
| | ATGTGATAACACGTGCATGGAAAGTGTCAAAAATGGGACTTATGACT | |
| | ACCCAAAATACTCAGAGGAAGCAAAATTAAACAGAGAAGAAATAGAT | |
| | GGGGTAAAGCTGGAATCAACAAGGATTTACCAGATTTTGGCGATCTA | |
| | TTCAACCGTCGCCAGTTCATTGGTACTGGTAGTCTCCCTGGGGGCAA | |
| | TCAGTTTCTGGATGTGCTCTAATGGGTCTCTACAGTGTAGAATATGTA | |
| | TTTAAGGAAGCGGAGCCACGAACTTCTCTCTGTTAAAGCAAGCAGGA | |
| | GATGTTGAAGAAAACCCCGGGCCTAGCAAAAGCAGGGGATAATTCTA | |
| | TTAACCATGAAGACTATCATTGCTTTGAGCTACATTCTATGTCTGGTTT | |
| | TCGCTCAAAAACTTCCTGGAAATGACAATAGCACTGCAACGCTGTGC | |
| | CTTGGGCACCATGCAGTACCAAACGGAACGATAGTGAAAACAATCAC | |
| | GAATGACCAAATTGAAGTTACTAATGCTACTGAGCTGGTTCAGAATTC | |
| | CTCAATAGGTGAAATATGCGACAGTCCTCATCAGATCCTTGATGGAG | |
| | AAAACTGCACACTAATAGATGCTCTATTGGGAGACCCTCAGTGTGAT | |
| | GGCTTTCAAAATAAGAAATGGGACCTTTTTGTTGAACGAAGCAAAGC | |
| | CTACAGCAACTGTTACCCTTATGATGTGCCGGATTATGCCTCCCTTAG | |
| | GTCACTAGTTGCCTCATCCGGCACACTGGAGTTTAACAATGAAAGCT | |
| | TCAATTGGGCTGGAGTCACTCAAAACGGAACAAGTTCTGCTTGCATA | |
| | AGGGGATCTAATAGTAGTTTCTTTAGTAGATTAAATTGGTTGACCCAC | |
| | TTAAACTTCAAGTACCCAGCATTGAACGTGACTATGCCAAACAATGAA | |
| | CAATTTGACAAATTGTACATTTGGGGGGTTCACCACCCGGGTACGGA | |
| | CAAGGACCAAATCTTCCTGTATGCTCAATCATCAGGAAGAATCACAGT | |
| | ATCTACCAAAAGAAGCCAACAAGCTGTAATCCCGAATATCGGATCTA | |
| | GACCCAGAATAAGGAATATCCCTAGCAGAATAAGCATCTATTGGACA | |
| | ATAGTAAAACCGGGAGACATACTTTTGATTAACAGCACAGGGAATCTA | |
| | ATTGCTCCTAGGGGTTACTTCAAAATACGAAGTGGGAAAAGCTCAAT | |
| | AATGAGATCAGATGCACCCATTGGCAAATGCAAGTCTGAATGCATCA | |
| | CTCCAAATGGAAGCATTCCCAATGACAAACCATTCCAAAATGTAAACA | |
| | GGATCACATACGGGGCCTGTCCCAGATATGTTAAGCAAAGCACTCTG | |
| | AAATTGGCAACAGGAATGCGAAATGTACCAGAGAAACAAACTAGAGG | |
| | CATATTTGGCGCAATAGCGGGTTTCATAGAAAATGGTTGGGAGGGAA | |
| | TGGTGGATGGTTGGTACGGTTTCAGGCATCAAAATTCTGAGGGAAGA | |
| | GGACAAGCAGCAGATCTCAAAAGCACTCAAGCAGCAATCGATCAAAT | |
| | CAATGGGAAGCTGAATCGATTGATCGGGAAAACCAACGAGAAATTCC | |
| | ATCAGATTGAGAAAGAATTCTCAGAAGTAGAAGGGAGAATTCAGGAC | |
| | CTTGAGAAATATGTTGAGGACACAAAAATAGATCTCTGGTCATACAAC | |
| | GCAGAGCTTCTTGTTGCCCTGGAAAACCAACATACAATTGATCTAACT | |
| | GACTGAGAAATGAACAAACTGTTTGAAAAAACAAAGAAGCAACTGAG | |
| | GGAAAATGCTGAGGATATGGGCAATGGTTGTTTCAAAATATACCACA | |
| | AATGTGACAATGCCTGCATAGGATCAATCAGAAATGGAACTTATGAC | |
| | CACGATGTATACAGGGATGAAGCATTAAACAACCGGTTCCAGATCAA | |
| | GGGAGTTGAGCTGAAGTCAGGGTACAAAGATTGGATCCTATGGATTT | |
| | CCTTTGCCATATCATGTTTTTTGCTTTGTGTTGCTTTGTTGGGGTTCAT | |
| | CATGTGGGCCTGCCAAAAGGGCAACATTAGGTGCAACATTTGCATTT | |
| | GAGTGCATTAATTAAAAACACCCTTGTTTCTACTGGAAGCGGAGCCA | |
| | CGAACTTCTCTCTGTTAAAGCAAGCAGGAGATGTTGAAGAAAACCCC | |
| | GGGCCTATTTTCTAATATCCACAAAATGAAGGCAATAATTGTACTACT | |
| | CATGGTAGTAACATCCAATGCAGATCGAATCTGCACTGGGATAACAT | |
| | CGTCAAACTCACCACATGTCGTCAAAACTGCTACTCAAGGGGAGGTC | |
| | AACGTGACCGGTGTAATACCACTGACAACAACACCCACCAAATCTCA | |
| | TTTTGCAAATCTCAAAGGAACAGAAACCAGGGGGAAACTATGCCCAA | |
| | AATGCCTCAACTGCACAGATCTGGATGTAGCCTTGGGCAGACCAAAA | |
| | TGCACAGGGAAAATACCCTCTGCAAGGGTTTCAATACTCCATGAAGT | |
| | CAGACCTGTTACATCTGGGTGCTTTCCTATAATGCACGATAGAACAAA | |
| | AATTAGACAGCTGCCTAACCTTCTCCGAGGATACGAACATGTCAGGT | |
| | TATCAACTCACAACGTTATCAATGCAAAAGATGCACCAGGAAGACCC | |
| | TACGAAATTGGAACCTCAGGGTCTTGCCCTAACATTACCAATGGAAA | |
| | CGGATTCTTCGCAACAATGGCTTGGGCCGTCCCAAAAAACAAAACAG | |
| | CAACAAATCCATTAACAATAGAAGTACCATACATTTGTACAGAAGGAG | |
| | AAGACCAAATTACCGTTTGGGGGTTCCACTCTGACAACGAGACCCAA | |
| | ATGGCAAAGCTCTATGGGGACTCAAAGCCCCAGAAGTTCACCTCATC | |
| | TGCCAACGGAGTGACCACACATTACGTTTCACAGATTGGTGGCTTCC | |
| | CAAATCAAACAGAAGACGGAGGACTACCACAAAGTGGCAGAATTGTT | |
| | GTTGATTACATGGTGCAGAAATCTGGAAAAACAGGAACAATTACCTAT | |
| | CAAAGAGGTATTTTATTGCCTCAAAAAGTGTGGTGCGCAAGTGGCAG | |
| | GAGCAAGGTAATAAAAGGATCCTTGCCCTTAATTGGAGAAGCAGATT | |
| | GCCTCCATGAAAAATACGGTGGATTAAACAAAAGCAAGCCTTACTAC | |
| | ACAGGGGAACATGCAAAGGCCATAGGAAATTGCCCAATATGGGTGAA | |
| | AACACCCTTGAAGCTGGCCAATGGAACCAAATATAGACCCCCTGCAA | |
| | AACTATTAAAGGAAAGAGGTTTCTTCGGAGCCATTGCTGGTTTCTTAG | |
| | AGGGAGGATGGGAAGGAATGATTGCAGGTTGGCACGGATACACATC | |
| | CCATGGGGCACATGGAGTAGCGGTGGCAGCTGACCTTAAGAGCACT | |
| | CAAGAGGCCATAAACAAGATAACAAAAAATCTCAACTCTTTGAGTGAG | |
| | CTGGAAGTAAAGAATCTTCAAAGACTAAGCGGTGCCATGGATGAACT | |
| | CCACAACGAAATACTAGAACTAGATGAGAAAGTGGATGATCTCAGAG | |
| | CTGACACAATAAGCTCACAAATAGAACTCGCAGTCCTGCTTTCCAATG | |
| | AAGGAATAATAAACAGTGAAGATGAACATCTCTTGGCGCTTGAAAGA | |
| | AAGCTGAAGAAAATGCTGGGCCCCTCTGCTGTAGAGATAGGGAATG | |
| | GATGCTTTGAAACCAAACACAAGTGCAACCAGACCTGCCTCGACAGA | |
| | ATAGCTGCTGGTACCTTTGATGCAGGAGAATTTTCTCTCCCCACCTTT | |
| | GATTCACTGAATATTACTGCTGCATCTTTAAATGACGACGGATTGGAC | |
| | AATCATACTATACTGCTTTACTACTCAACTGCTGCCTCCAGTTTGGCT | |
| | GTAACACTGATGATAGCTATCTTTGTTGTTTATATGGTCTCCAGAGAC | |
| | AATGTTTCTTGCTCCATTTGTCTATAAGGAAAGTTAAGCCCTGTATTTT | |
| | CCTTTATTGTAGTGCTTGTTTGCTTGTTGTCATTACAAAGAAACGTTAT | |
| | TGAAAAATGGAAGCGGAGCCACGAACTTCTCTCTGTTAAAGCAAGCA | |
| | GGAGATGTTGAAGAAAACCCCGGGCCTTTCGGAGCTATTGCTGGTTT | |
| | CTTGGAAGGAGGATGGGAAGGAATGATTGCAGGTTGGCACGGATAC | |
| | ACATCTCATGGAGCACATGGAGTAGCAGTGGCAGCAGACCTTAAGA | |
| | GTACCCATGATGACTCGAGCTGGTACTGCATGCACGCAATGCTAGCT | |
| | GCCCCTTTCCCGTCCTGGGTACCCCGAGTCTCCCCCGACCTCGGGT | |
| | CCCAGGTATGCTCCCACCTCCACCTGCCCCACTCACCACCTCTGCTA | |
| | GTTCCAGACACCTCCCAAGCACGCAGCAATGCAGCTCAAAACGCTTA | |
| | GCCTAGCCACACCCCCACGGGAAACAGCAGTGATTAACCTTTAGCAA | |
| | TAAACGAAAGTTTAACTAAGCTATACTAACCCCAGGGTTGGTCAATTT | |
| | CGTGCCAGCCACACCCTGGAGCTAGCAAAAAAAA | |
| Pan-CoV-Flu | CTCGACATTGATTATTGACTAGTTATTAATAGTAATCAATTACGGGGT | 398 |
| Vaccine #10 | CATTAGTTCATAGCCCATATATGGAGTTCCGCGTTACATAACTTACGG | |
| CoV-Omicron | TAAATGGCCCGCCTGGCTGACCGCCCAACGACCCCCGCCCATTGAC | |
| BA.2 Spike(2 | GTCAATAATGACGTATGTTCCCATAGTAACGCCAATAGGGACTTTCCA | |
| proline | TTGACGTCAATGGGTGGAGTATTTACGGTAAACTGCCCACTTGGCAG | |
| substitution)- | TACATCAAGTGTATCATATGCCAAGTACGCCCCCTATTGACGTCAATG | |
| Influenza (HA) | ACGGTAAATGGCCCGCCTGGCATTATGCCCAGTACATGACCTTATGG | |
| | GACTTTCCTACTTGGCAGTACATCTACGTATTAGTCATCGCTATTACC | |
| | ATGGTCGAGGTGAGCCCCACGTTCTGCTTCACTCTCCCCATCTCCCC | |
| | CCCCTCCCCACCCCCAATTTTGTATTTATTTATTTTTTAATTATTTTGT | |
| | GCAGCGATGGGGGCGGGGGGGGGGGGGGGGCGCGCGCCAGGCG | |
| | GGGCGGGGCGGGGCGAGGGGCGGGGCGGGGCGAGGCGGAGAGG | |
| | TGCGGCGGCAGCCAATCAGAGCGGCGCGCTCCGAAAGTTTCCTTTT | |
| | ATGGCGAGGCGGCGGCGGCGGCGGCCCTATAAAAAGCGAAGCGCG | |
| | CGGCGGGCGGGAGTCGCTGCGCGCTGCCTTCGCCCCGTGCCCCGC | |
| | TCCGCCGCCGCCTCGCGCCGCCCGCCCCGGCTCTGACTGACCGCG | |
| | TTACTCCCACAGGTGAGCGGGCGGGACGGCCCTTCTCCTCCGGGCT | |
| | GTAATTAGCGCTTGGTTTAATGACGGCTTGTTTCTTTTCTGTGGCTGC | |
| | GTGAAAGCCTTGAGGGGCTCCGGGAGGGCCCTTTGTGCGGGGGGA | |
| | GCGGCTCGGGGGGTGCGTGCGTGTGTGTGTGCGTGGGGAGCGCCG | |
| | CGTGCGGCTCCGCGCTGCCCGGCGGCTGTGAGCGCTGCGGGCGCG | |
| | GCGCGGGGCTTTGTGCGCTCCGCAGTGTGCGCGAGGGGAGCGCGG | |
| | CCGGGGGCGGTGCCCCGCGGTGCGGGGGGGGCTGCGAGGGGAAC | |
| | AAAGGCTGCGTGCGGGGTGTGTGCGTGGGGGGGTGAGCAGGGGGT | |
| | GTGGGCGCGTCGGTCGGGCTGCAACCCCCCCTGCACCCCCCTCCC | |
| | CGAGTTGCTGAGCACGGCCCGGCTTCGGGTGCGGGGCTCCGTACG | |
| | GGGCGTGGCGCGGGGCTCGCCGTGCCGGGCGGGGGGTGGCGGCA | |
| | GGTGGGGGTGCCGGGCGGGGCGGGGCCGCCTCGGGCCGGGGAG | |
| | GGCTCGGGGGAGGGGCGCGGCGGCCCCCGGAGCGCCGGCGGCTG | |
| | TCGAGGCGCGGCGAGCCGCAGCCATTGCCTTTTATGGTAATCGTGC | |
| | GAGAGGGCGCAGGGACTTCCTTTGTCCCAAATCTGTGCGGAGCCGA | |
| | AATCTGGGAGGCGCCGCCGCACCCCCTCTAGCGGGCGCGGGGCGA | |
| | AGCGGTGCGGCGCCGGCAGGAAGGAAATGGGCGGGGAGGGCCTTC | |
| | GTGCGTCGCCGCGCCGCCGTCCCCTTCTCCCTCTCCAGCCTCGGGG | |
| | CTGTCCGCGGGGGGACGGCTGCCTTCGGGGGGGACGGGGCAGGG | |
| | CGGGGTTCGGCTTCTGGCGTGTGACCGGCGGCTCTAGAGCCTCTGC | |
| | TAACCATGTTCATGCCTTCTTCTTTTTCCTACAGCTCCTGGGCAACGT | |
| | GCTGGTTATTGTGCTGTCTCATCATTTTGGCAAAGAATTGGAGAATAA | |
| | ACTAGTATTCTTCTGGTCCCCACAGACTCAGAGAGAACCCGCCACCA | |
| | TGTTCGTGTTCCTGGTGCTGCTGCCCCTGGTGAGCAGCCAGTGCGT | |
| | GAACCTGATCACCAGGACCCAGAGCTACACCAACAGCTTCACCAGG | |
| | GGCGTGTACTACCCCGACAAGGTGTTCAGGAGCAGCGTGCTGCACA | |
| | GCACCCAGGACCTGTTCCTGCCCTTCTTCAGCAACGTGACCTGGTTC | |
| | CACGCCATCCACGTGAGCGGCACCAACGGCACCAAGAGGTTCGACA | |
| | ACCCCGTGCTGCCCTTCAACGACGGCGTGTACTTCGCCAGCACCGA | |
| | GAAGAGCAACATCATCAGGGGCTGGATCTTCGGCACCACCCTGGAC | |
| | AGCAAGACCCAGAGCCTGCTGATCGTGAACAACGCCACCAACGTGG | |
| | TGATCAAGGTGTGCGAGTTCCAGTTCTGCAACGACCCCTTCCTGGAC | |
| | GTGTACTACCACAAGAACAACAAGAGCTGGATGGAGAGCGAGTTCA | |
| | GGGTGTACAGCAGCGCCAACAACTGCACCTTCGAGTACGTGAGCCA | |
| | GCCCTTCCTGATGGACCTGGAGGGCAAGCAGGGCAACTTCAAGAAC | |
| | CTGAGGGAGTTCGTGTTCAAGAACATCGACGGCTACTTCAAGATCTA | |
| | CAGCAAGCACACCCCCATCAACCTGGGCAGGGACCTGCCCCAGGGC | |
| | TTCAGCGCCCTGGAGCCCCTGGTGGACCTGCCCATCGGCATCAACA | |
| | TCACCAGGTTCCAGACCCTGCTGGCCCTGCACAGGAGCTACCTGAC | |
| | CCCCGGCGACAGCAGCAGCGGCTGGACCGCCGGCGCCGCCGCCTA | |
| | CTACGTGGGCTACCTGCAGCCCAGGACCTTCCTGCTGAAGTACAAC | |
| | GAGAACGGCACCATCACCGACGCCGTGGACTGCGCCCTGGACCCC | |
| | CTGAGCGAGACCAAGTGCACCCTGAAGAGCTTCACCGTGGAGAAGG | |
| | GCATCTACCAGACCAGCAACTTCAGGGTGCAGCCCACCGAGAGCAT | |
| | CGTGAGGTTCCCCAACATCACCAACCTGTGCCCCTTCGACGAGGTGT | |
| | TCAACGCCACCAGGTTCGCCAGCGTGTACGCCTGGAACAGGAAGAG | |
| | GATCAGCAACTGCGTGGCCGACTACAGCGTGCTGTACAACTTCGCC | |
| | CCCTTCTTCGCCTTCAAGTGCTACGGCGTGAGCCCCACCAAGCTGAA | |
| | CGACCTGTGCTTCACCAACGTGTACGCCGACAGCTTCGTGATCAGG | |
| | GGCAACGAGGTGAGCCAGATCGCCCCCGGCCAGACCGGCAACATC | |
| | GCCGACTACAACTACAAGCTGCCCGACGACTTCACCGGCTGCGTGA | |
| | TCGCCTGGAACAGCAACAAGCTGGACAGCAAGGTGGGCGGCAACTA | |
| | CAACTACCTGTACAGGCTGTTCAGGAAGAGCAACCTGAAGCCCTTCG | |
| | AGAGGGACATCAGCACCGAGATCTACCAGGCCGGCAACAAGCCCTG | |
| | CAACGGCGTGGCCGGCTTCAACTGCTACTTCCCCCTGAGGAGCTAC | |
| | GGCTTCAGGCCCACCTACGGCGTGGGCCACCAGCCCTACAGGGTG | |
| | GTGGTGCTGAGCTTCGAGCTGCTGCACGCCCCCGCCACCGTGTGCG | |
| | GCCCCAAGAAGAGCACCAACCTGGTGAAGAACAAGTGCGTGAACTT | |
| | CAACTTCAACGGCCTGACCGGCACCGGCGTGCTGACCGAGAGCAAC | |
| | AAGAAGTTCCTGCCCTTCCAGCAGTTCGGCAGGGACATCGCCGACA | |
| | CCACCGACGCCGTGAGGGACCCCCAGACCCTGGAGATCCTGGACAT | |
| | CACCCCCTGCAGCTTCGGCGGCGTGAGCGTGATCACCCCCGGCACC | |
| | AACACCAGCAACCAGGTGGCCGTGCTGTACCAGGGCGTGAACTGCA | |
| | CCGAGGTGCCCGTGGCCATCCACGCCGACCAGCTGACCCCCACCTG | |
| | GAGGGTGTACAGCACCGGCAGCAACGTGTTCCAGACCAGGGCCGG | |
| | CTGCCTGATCGGCGCCGAGTACGTGAACAACAGCTACGAGTGCGAC | |
| | ATCCCCATCGGCGCCGGCATCTGCGCCAGCTACCAGACCCAGACCA | |
| | AGAGCCACCAGCAGGCCCAGAGCGTGGCCAGCCAGAGCATCATCG | |
| | CCTACACCATGAGCCTGGGCGCCGAGAACAGCGTGGCCTACAGCAA | |
| | CAACAGCATCGCCATCCCCACCAACTTCACCATCAGCGTGACCACCG | |
| | AGATCCTGCCCGTGAGCATGACCAAGACCAGCGTGGACTGCACCAT | |
| | GTACATCTGCGGCGACAGCACCGAGTGCAGCAACCTGCTGCTGCAG | |
| | TACGGCAGCTTCTGCACCCAGCTGAAGAGGGCCCTGACCGGCATCG | |
| | CCGTGGAGCAGGACAAGAACACCCAGGAGGTGTTCGCCCAGGTGAA | |
| | GCAGATCTACAAGACCCCCCCCATCAAGTACTTCGGCGGCTTCAACT | |
| | TCAGCCAGATCCTGCCCGACCCCAGCAAGCCCAGCAAGAGGAGCTT | |
| | CATCGAGGACCTGCTGTTCAACAAGGTGACCCTGGCCGACGCCGGC | |
| | TTCATCAAGCAGTACGGCGACTGCCTGGGCGACATCGCCGCCAGGG | |
| | ACCTGATCTGCGCCCAGAAGTTCAACGGCCTGACCGTGCTGCCCCC | |
| | CCTGCTGACCGACGAGATGATCGCCCAGTACACCAGCGCCCTGCTG | |
| | GCCGGCACCATCACCAGCGGCTGGACCTTCGGCGCCGGCGCCGCC | |
| | CTGCAGATCCCCTTCGCCATGCAGATGGCCTACAGGTTCAACGGCAT | |
| | CGGCGTGACCCAGAACGTGCTGTACGAGAACCAGAAGCTGATCGCC | |
| | AACCAGTTCAACAGCGCCATCGGCAAGATCCAGGACAGCCTGAGCA | |
| | GCACCGCCAGCGCCCTGGGCAAGCTGCAGGACGTGGTGAACCACA | |
| | ACGCCCAGGCCCTGAACACCCTGGTGAAGCAGCTGAGCAGCAAGTT | |
| | CGGCGCCATCAGCAGCGTGCTGAACGACATCCTGAGCAGGCTGGAC | |
| | CCCCCCGAGGCCGAGGTGCAGATCGACAGGCTGATCACCGGCAGG | |
| | CTGCAGAGCCTGCAGACCTACGTGACCCAGCAGCTGATCAGGGCCG | |
| | CCGAGATCAGGGCCAGCGCCAACCTGGCCGCCACCAAGATGAGCG | |
| | AGTGCGTGCTGGGCCAGAGCAAGAGGGTGGACTTCTGCGGCAAGG | |
| | GCTACCACCTGATGAGCTTCCCCCAGAGCGCCCCCCACGGCGTGGT | |
| | GTTCCTGCACGTGACCTACGTGCCCGCCCAGGAGAAGAACTTCACC | |
| | ACCGCCCCCGCCATCTGCCACGACGGCAAGGCCCACTTCCCCAGGG | |
| | AGGGCGTGTTCGTGAGCAACGGCACCCACTGGTTCGTGACCCAGAG | |
| | GAACTTCTACGAGCCCCAGATCATCACCACCGACAACACCTTCGTGA | |
| | GCGGCAACTGCGACGTGGTGATCGGCATCGTGAACAACACCGTGTA | |
| | CGACCCCCTGCAGCCCGAGCTGGACAGCTTCAAGGAGGAGCTGGAC | |
| | AAGTACTTCAAGAACCACACCAGCCCCGACGTGGACCTGGGCGACA | |
| | TCAGCGGCATCAACGCCAGCGTGGTGAACATCCAGAAGGAGATCGA | |
| | CAGGCTGAACGAGGTGGCCAAGAACCTGAACGAGAGCCTGATCGAC | |
| | CTGCAGGAGCTGGGCAAGTACGAGCAGTACATCAAGTGGCCCTGGT | |
| | ACATCTGGCTGGGCTTCATCGCCGGCCTGATCGCCATCGTGATGGT | |
| | GACCATCATGCTGTGCTGCATGACCAGCTGCTGCAGCTGCCTGAAG | |
| | GGCTGCTGCAGCTGCGGCAGCTGCTGCAAGTTCGACGAGGACGACA | |
| | GCGAGCCCGTGCTGAAGGGCGTGAAGCTGCACTACACCGGAAGCG | |
| | GAGCCACGAACTTCTCTCTGTTAAAGCAAGCAGGAGATGTTGAAGAA | |
| | AACCCCGGGCCTATGAAGGCAATACTAGTAGTTCTGCTATATACATTT | |
| | GCAACCGCAAATGCAGACACATTATGTATAGGTTATCATGCGAACAA | |
| | CTCAACAGACACTGTAGACACAGTACTAGAAAAGAATGTAACAGTAA | |
| | CACACTCTGTTAACCTTCTAGAAGACAAGCATAACGGGAAACTATGC | |
| | AAACTAAGAGGGGTAGCCCCATTGCATTTGGGTAAATGTAACATTGC | |
| | TGGCTGGATCCTGGGAAATCCAGAGTGTGAATCACTCTCCACAGCAA | |
| | GCTCATGGTCCTACATTGTGGAAACATCTAGTTCAGACAATGGAACG | |
| | TGTTACCCAGGAGATTTCATCGATTATGAGGAGCTAAGAGAGCAATT | |
| | GAGCTCAGTGTCATCATTTGAAAGGTTTGAGATATTCCCCAAGACAA | |
| | GTTCATGGCCCAATCATGACTCGAACAAAGGTGTAACGACAGCATGT | |
| | CCTCATGCTGGAGCAAAAAGCTTCTACAAAAATTTAATATGGCTAGTT | |
| | AAAAAAGGAAATTCATACCCAAAGCTCAGCAAATCCTACATTAATGAT | |
| | AAAGGGAAAGAAGTCCTCGTGCTATGGGGCATTCACCATCCACCTAC | |
| | TAGTGCTGACCAACAAAGTCTCTATCAGAATGCAGATGCATATGTTTT | |
| | TGTGGGGACATCAAGATACAGCAAGAAGTTCAAGCCGGAAATAGCAA | |
| | TAAGGCCCAAAGTGAGGGATCAAGAAGGGAGAATGAACTATTACTGG | |
| | ACACTAGTAGAGCCGGGAGACAAAATAACATTCGAAGCAACTGGAAA | |
| | TCTAGTGGTACCGAGATATGCATTCGCAATGGAAAGAAATGCTGGAT | |
| | CTGGTATTATCATTTCAGATACACCAGTCCACGATTGCAATACAACTT | |
| | GTCAGACACCCAAGGGTGCTATAAACACCAGCCTCCCATTTCAGAAC | |
| | ATACATCCGATCACAATTGGAAAATGTCCAAAATATGTAAAAAGCACA | |
| | AAATTGAGACTGGCCACAGGATTGAGGAATGTCCCGTCCATTCAATC | |
| | TAGAGGCCTATTTGGGGCCATTGCCGGTTTCATTGAAGGGGGGTGG | |
| | ACAGGGATGGTAGATGGATGGTACGGTTATCACCATCAAAATGAGCA | |
| | GGGGTCAGGATATGCAGCCGACCTGAAGAGCACACAGAATGCCATT | |
| | GACGAGATTACTAACAAAGTAAACTCTGTTATTGAAAAAATGAATACA | |
| | CAGTTCACAGCAGTAGGTAAAGAGTTCAACCACCTGGAAAAAAGAAT | |
| | AGAGAATTTAAATAAAAAAGTTGATGATGGTTTCCTGGACATTTGGAC | |
| | TTACAATGCCGAACTGTTGGTTCTATTGGAAAATGAAAGAACTTTGGA | |
| | CTACCACGATTCAAAGGTGAAGAACTTATATGAAAAGGTAAGAAGCC | |
| | AGTTAAAAAACAATGCCAAGGAAATTGGAAACGGCTGCTTTGAATTTT | |
| | ACCACAAATGTGATAACACGTGCATGGAAAGTGTCAAAAATGGGACT | |
| | TATGACTACCCAAAATACTCAGAGGAAGCAAAATTAAACAGAGAAGAA | |
| | ATAGATGGGGTAAAGCTGGAATCAACAAGGATTTACCAGATTTTGGC | |
| | GATCTATTCAACCGTCGCCAGTTCATTGGTACTGGTAGTCTCCCTGG | |
| | GGGCAATCAGTTTCTGGATGTGCTCTAATGGGTCTCTACAGTGTAGA | |
| | ATATGTATTTAAGGAAGCGGAGCCACGAACTTCTCTCTGTTAAAGCAA | |
| | GCAGGAGATGTTGAAGAAAACCCCGGGCCTAGCAAAAGCAGGGGAT | |
| | AATTCTATTAACCATGAAGACTATCATTGCTTTGAGCTACATTCTATGT | |
| | CTGGTTTTCGCTCAAAAACTTCCTGGAAATGACAATAGCACTGCAAC | |
| | GCTGTGCCTTGGGCACCATGCAGTACCAAACGGAACGATAGTGAAAA | |
| | CAATCACGAATGACCAAATTGAAGTTACTAATGCTACTGAGCTGGTTC | |
| | AGAATTCCTCAATAGGTGAAATATGCGACAGTCCTCATCAGATCCTTG | |
| | ATGGAGAAAACTGCACACTAATAGATGCTCTATTGGGAGACCCTCAG | |
| | TGTGATGGCTTTCAAAATAAGAAATGGGACCTTTTTGTTGAACGAAGC | |
| | AAAGCCTACAGCAACTGTTACCCTTATGATGTGCCGGATTATGCCTC | |
| | CCTTAGGTCACTAGTTGCCTCATCCGGCACACTGGAGTTTAACAATG | |
| | AAAGCTTCAATTGGGCTGGAGTCACTCAAAACGGAACAAGTTCTGCT | |
| | TGCATAAGGGGATCTAATAGTAGTTTCTTTAGTAGATTAAATTGGTTG | |
| | ACCCACTTAAACTTCAAGTACCCAGCATTGAACGTGACTATGCCAAAC | |
| | AATGAACAATTTGACAAATTGTACATTTGGGGGGTTCACCACCCGGG | |
| | TACGGACAAGGACCAAATCTTCCTGTATGCTCAATCATCAGGAAGAA | |
| | TCACAGTATCTACGAAAAGAAGCCAACAAGCTGTAATGCGGAATATC | |
| | GGATCTAGACCCAGAATAAGGAATATCCCTAGCAGAATAAGCATCTA | |
| | TTGGACAATAGTAAAACCGGGAGACATACTTTTGATTAACAGCACAG | |
| | GGAATCTAATTGCTCCTAGGGGTTACTTCAAAATACGAAGTGGGAAA | |
| | AGCTCAATAATGAGATCAGATGCACCCATTGGCAAATGCAAGTCTGA | |
| | ATGCATCACTCCAAATGGAAGCATTCCCAATGACAAACCATTCCAAAA | |
| | TGTAAACAGGATCACATACGGGGCCTGTCCCAGATATGTTAAGCAAA | |
| | GCACTCTGAAATTGGCAACAGGAATGCGAAATGTACCAGAGAAACAA | |
| | ACTAGAGGCATATTTGGCGCAATAGCGGGTTTCATAGAAAATGGTTG | |
| | GGAGGGAATGGTGGATGGTTGGTACGGTTTCAGGCATCAAAATTCTG | |
| | AGGGAAGAGGACAAGCAGCAGATCTCAAAAGCACTCAAGCAGCAAT | |
| | CGATCAAATCAATGGGAAGCTGAATCGATTGATCGGGAAAACCAACG | |
| | AGAAATTCCATCAGATTGAGAAAGAATTCTCAGAAGTAGAAGGGAGA | |
| | ATTCAGGACCTTGAGAAATATGTTGAGGACACAAAAATAGATCTCTGG | |
| | TCATACAACGCAGAGCTTCTTGTTGCCCTGGAAAACCAACATACAATT | |
| | GATCTAACTGACTCAGAAATGAACAAACTGTTTGAAAAAACAAAGAAG | |
| | CAACTGAGGGAAAATGCTGAGGATATGGGCAATGGTTGTTTCAAAAT | |
| | ATACCACAAATGTGACAATGCCTGCATAGGATCAATCAGAAATGGAA | |
| | CTTATGACCACGATGTATACAGGGATGAAGCATTAAACAACCGGTTC | |
| | CAGATCAAGGGAGTTGAGCTGAAGTCAGGGTACAAAGATTGGATCCT | |
| | ATGGATTTCCTTTGCCATATCATGTTTTTTGCTTTGTGTTGCTTTGTTG | |
| | GGGTTCATCATGTGGGCCTGCCAAAAGGGCAACATTAGGTGCAACAT | |
| | TTGCATTTGAGTGCATTAATTAAAAACACCCTTGTTTCTACTGGAAGC | |
| | GGAGCCACGAACTTCTCTCTGTTAAAGCAAGCAGGAGATGTTGAAGA | |
| | AAACCCCGGGCCTATTTTCTAATATCCACAAAATGAAGGCAATAATTG | |
| | TACTACTCATGGTAGTAACATCCAATGCAGATCGAATCTGCACTGGG | |
| | ATAACATCGTCAAACTCACCACATGTCGTCAAAACTGCTACTCAAGG | |
| | GGAGGTCAACGTGACCGGTGTAATACCACTGACAACAACACCCACCA | |
| | AATCTCATTTTGCAAATCTCAAAGGAACAGAAACCAGGGGGAAACTAT | |
| | GCCCAAAATGCCTCAACTGCACAGATCTGGATGTAGCCTTGGGCAGA | |
| | CCAAAATGCACAGGGAAAATACCCTCTGCAAGGGTTTCAATACTCCA | |
| | TGAAGTCAGACCTGTTACATCTGGGTGCTTTCCTATAATGCACGATAG | |
| | AACAAAAATTAGACAGCTGCCTAACCTTCTCCGAGGATACGAACATG | |
| | TCAGGTTATCAACTCACAACGTTATCAATGCAAAAGATGCACCAGGAA | |
| | GACCCTACGAAATTGGAACCTCAGGGTCTTGCCCTAACATTACCAAT | |
| | GGAAACGGATTCTTCGCAACAATGGCTTGGGCCGTCCCAAAAAACAA | |
| | AACAGCAACAAATCCATTAACAATAGAAGTACCATACATTTGTACAGA | |
| | AGGAGAAGACCAAATTACCGTTTGGGGGTTCCACTCTGACAACGAGA | |
| | CCCAAATGGCAAAGCTCTATGGGGACTCAAAGCCCCAGAAGTTCACC | |
| | TCATCTGCCAACGGAGTGACCACACATTACGTTTCACAGATTGGTGG | |
| | CTTCCCAAATCAAACAGAAGACGGAGGACTACCACAAAGTGGCAGAA | |
| | TTGTTGTTGATTACATGGTGCAGAAATCTGGAAAAACAGGAACAATTA | |
| | CCTATCAAAGAGGTATTTTATTGCCTCAAAAAGTGTGGTGCGCAAGT | |
| | GGCAGGAGCAAGGTAATAAAAGGATCCTTGCCCTTAATTGGAGAAGC | |
| | AGATTGCCTCCATGAAAAATACGGTGGATTAAACAAAAGCAAGCCTTA | |
| | CTACACAGGGGAACATGCAAAGGCCATAGGAAATTGCCCAATATGGG | |
| | TGAAAACACCCTTGAAGCTGGCCAATGGAACCAAATATAGACCCCCT | |
| | GCAAAACTATTAAAGGAAAGAGGTTTCTTCGGAGCCATTGCTGGTTT | |
| | CTTAGAGGGAGGATGGGAAGGAATGATTGCAGGTTGGCACGGATAC | |
| | ACATCCCATGGGGCACATGGAGTAGCGGTGGCAGCTGACCTTAAGA | |
| | GCACTCAAGAGGCCATAAACAAGATAACAAAAAATCTCAACTCTTTGA | |
| | GTGAGCTGGAAGTAAAGAATCTTCAAAGACTAAGCGGTGCCATGGAT | |
| | GAACTCCACAACGAAATACTAGAACTAGATGAGAAAGTGGATGATCT | |
| | CAGAGCTGACACAATAAGCTCACAAATAGAACTCGCAGTCCTGCTTT | |
| | CCAATGAAGGAATAATAAACAGTGAAGATGAACATCTCTTGGCGCTT | |
| | GAAAGAAAGCTGAAGAAAATGCTGGGCCCCTCTGCTGTAGAGATAG | |
| | GGAATGGATGCTTTGAAACCAAACACAAGTGCAACCAGACCTGCCTC | |
| | GACAGAATAGCTGCTGGTACCTTTGATGCAGGAGAATTTTCTCTCCC | |
| | CACCTTTGATTCACTGAATATTACTGCTGCATCTTTAAATGACGACGG | |
| | ATTGGACAATCATACTATACTGCTTTACTACTCAACTGCTGCCTCCAG | |
| | TTTGGCTGTAACACTGATGATAGCTATCTTTGTTGTTTATATGGTCTC | |
| | CAGAGACAATGTTTCTTGCTCCATTTGTCTATAAGGAAAGTTAAGCCC | |
| | TGTATTTTCCTTTATTGTAGTGCTTGTTTGCTTGTTGTCATTACAAAGA | |
| | AACGTTATTGAAAAATGGAAGCGGAGCCACGAACTTCTCTCTGTTAAA | |
| | GCAAGCAGGAGATGTTGAAGAAAACCCCGGGCCTTTCGGAGCTATT | |
| | GCTGGTTTCTTGGAAGGAGGATGGGAAGGAATGATTGCAGGTTGGC | |
| | ACGGATACACATCTCATGGAGCACATGGAGTAGCAGTGGCAGCAGA | |
| | CCTTAAGAGTACCCATGATGACTCGAGCTGGTACTGCATGCACGCAA | |
| | TGCTAGCTGCCCCTTTCCCGTCCTGGGTACCCCGAGTCTCCCCCGA | |
| | CCTCGGGTCCCAGGTATGCTCCCACCTCCACCTGCCCCACTCACCA | |
| | CCTCTGCTAGTTCCAGACACCTCCCAAGCACGCAGCAATGCAGCTCA | |
| | AAACGCTTAGCCTAGCCACACCCCCACGGGAAACAGCAGTGATTAAC | |
| | CTTTAGCAATAAACGAAAGTTTAACTAAGCTATACTAACCCCAGGGTT | |
| | GGTCAATTTCGTGCCAGCCACACCCTGGAGCTAGCAAAAAAAA | |
| Pan-CoV-Flu | TAGTTATTAATAGTAATCAATTACGGGGTCATTAGTTCATAGCCCATAT | 399 |
| Vaccine #11 | ATGGAGTTCCGCGTTACATAACTTACGGTAAATGGCCCGCCTGGCTG | |
| CoV-Wuhan | ACCGCCCAACGACCCCCGCCCATTGACGTCAATAATGACGTATGTTC | |
| Spike(HexaPro)- | CCATAGTAACGCCAATAGGGACTTTCCATTGACGTCAATGGGTGGAG | |
| Influenza (HA) | TATTTACGGTAAACTGCCCACTTGGCAGTACATCAAGTGTATCATATG | |
| | CCAAGTACGCCCCCTATTGACGTCAATGACGGTAAATGGCCCGCCTG | |
| | GCATTATGCCCAGTACATGACCTTATGGGACTTTCCTACTTGGCAGTA | |
| | CATCTACGTATTAGTCATCGCTATTACCATGGTGATGCGGTTTTGGCA | |
| | GTACATCAATGGGCGTGGATAGCGGTTTGACTCACGGGGATTTCCAA | |
| | GTCTCCACCCCATTGACGTCAATGGGAGTTTGTTTTGGCACCAAAAT | |
| | CAACGGGACTTTCCAAAATGTCGTAACAACTCCGCCCCATTGACGCA | |
| | AATGGGCGGTAGGCGTGTACGGTGGGAGGTCTATATAAGCAGAGCT | |
| | GGTTTAGTGAACCGTCAGATCGAGAATAAACTAGTATTCTTCTGGTCC | |
| | CCACAGACTCAGAGAGAACCCGCCACCATGTTCGTGTTCCTGGTGCT | |
| | GCTGCCCCTGGTGAGCAGCCAGTGCGTGAACCTGACCACCAGGACC | |
| | CAGCTGCCCCCCGCCTACACCAACAGCTTCACCAGGGGCGTGTACT | |
| | ACCCCGACAAGGTGTTCAGGAGCAGCGTGCTGCACAGCACCCAGGA | |
| | CCTGTTCCTGCCCTTCTTCAGCAACGTGACCTGGTTCCACGCCATCC | |
| | ACGTGAGCGGCACCAACGGCACCAAGAGGTTCGACAACCCCGTGCT | |
| | GCCCTTCAACGACGGCGTGTACTTCGCCAGCACCGAGAAGAGCAAC | |
| | ATCATCAGGGGCTGGATCTTCGGCACCACCCTGGACAGCAAGACCC | |
| | AGAGCCTGCTGATCGTGAACAACGCCACCAACGTGGTGATCAAGGT | |
| | GTGCGAGTTCCAGTTCTGCAACGACCCCTTCCTGGGCGTGTACTACC | |
| | ACAAGAACAACAAGAGCTGGATGGAGAGCGAGTTCAGGGTGTACAG | |
| | CAGCGCCAACAACTGCACCTTCGAGTACGTGAGCCAGCCCTTCCTG | |
| | ATGGACCTGGAGGGCAAGCAGGGCAACTTCAAGAACCTGAGGGAGT | |
| | TCGTGTTCAAGAACATCGACGGCTACTTCAAGATCTACAGCAAGCAC | |
| | ACCCCCATCAACCTGGTGAGGGACCTGCCCCAGGGCTTCAGCGCCC | |
| | TGGAGCCCCTGGTGGACCTGCCCATCGGCATCAACATCACCAGGTT | |
| | CCAGACCCTGCTGGCCCTGCACAGGAGCTACCTGACCCCCGGCGAC | |
| | AGCAGCAGCGGCTGGACCGCCGGCGCCGCCGCCTACTACGTGGGC | |
| | TACCTGCAGCCCAGGACCTTCCTGCTGAAGTACAACGAGAACGGCA | |
| | CCATCACCGACGCCGTGGACTGCGCCCTGGACCCCCTGAGCGAGAC | |
| | CAAGTGCACCCTGAAGAGCTTCACCGTGGAGAAGGGCATCTACCAG | |
| | ACCAGCAACTTCAGGGTGCAGCCCACCGAGAGCATCGTGAGGTTCC | |
| | CCAACATCACCAACCTGTGCCCCTTCGGCGAGGTGTTCAACGCCAC | |
| | CAGGTTCGCCAGCGTGTACGCCTGGAACAGGAAGAGGATCAGCAAC | |
| | TGCGTGGCCGACTACAGCGTGCTGTACAACAGCGCCAGCTTCAGCA | |
| | CCTTCAAGTGCTACGGCGTGAGCCCCACCAAGCTGAACGACCTGTG | |
| | CTTCACCAACGTGTACGCCGACAGCTTCGTGATCAGGGGCGACGAG | |
| | GTGAGGCAGATCGCCCCCGGCCAGACCGGCAAGATCGCCGACTACA | |
| | ACTACAAGCTGCCCGACGACTTCACCGGCTGCGTGATCGCCTGGAA | |
| | CAGCAACAACCTGGACAGCAAGGTGGGCGGCAACTACAACTACCTG | |
| | TACAGGCTGTTCAGGAAGAGCAACCTGAAGCCCTTCGAGAGGGACA | |
| | TCAGCACCGAGATCTACCAGGCCGGCAGCACCCCCTGCAACGGCGT | |
| | GGAGGGCTTCAACTGCTACTTCCCCCTGCAGAGCTACGGCTTCCAG | |
| | CCCACCAACGGCGTGGGCTACCAGCCCTACAGGGTGGTGGTGCTGA | |
| | GCTTCGAGCTGCTGCACGCCCCCGCCACCGTGTGCGGCCCCAAGAA | |
| | GAGCACCAACCTGGTGAAGAACAAGTGCGTGAACTTCAACTTCAACG | |
| | GCCTGACCGGCACCGGCGTGCTGACCGAGAGCAACAAGAAGTTCCT | |
| | GCCCTTCCAGCAGTTCGGCAGGGACATCGCCGACACCACCGACGCC | |
| | GTGAGGGACCCCCAGACCCTGGAGATCCTGGACATCACCCCCTGCA | |
| | GCTTCGGCGGCGTGAGCGTGATCACCCCCGGCACCAACACCAGCAA | |
| | CCAGGTGGCCGTGCTGTACCAGGACGTGAACTGCACCGAGGTGCCC | |
| | GTGGCCATCCACGCCGACCAGCTGACCCCCACCTGGAGGGTGTACA | |
| | GCACCGGCAGCAACGTGTTCCAGACCAGGGCCGGCTGCCTGATCG | |
| | GCGCCGAGCACGTGAACAACAGCTACGAGTGCGACATCCCCATCGG | |
| | CGCCGGCATCTGCGCCAGCTACCAGACCCAGACCAACAGCCCCGGC | |
| | AGCGCCAGCAGCGTGGCCAGCCAGAGCATCATCGCCTACACCATGA | |
| | GCCTGGGCGCCGAGAACAGCGTGGCCTACAGCAACAACAGCATCGC | |
| | CATCCCCACCAACTTCACCATCAGCGTGACCACCGAGATCCTGCCCG | |
| | TGAGCATGACCAAGACCAGCGTGGACTGCACCATGTACATCTGCGG | |
| | CGACAGCACCGAGTGCAGCAACCTGCTGCTGCAGTACGGCAGCTTC | |
| | TGCACCCAGCTGAACAGGGCCCTGACCGGCATCGCCGTGGAGCAG | |
| | GACAAGAACACCCAGGAGGTGTTCGCCCAGGTGAAGCAGATCTACA | |
| | AGACCCCCCCCATCAAGGACTTCGGCGGCTTCAACTTCAGCCAGATC | |
| | CTGCCCGACCCCAGCAAGCCCAGCAAGAGGAGCCCCATCGAGGAC | |
| | CTGCTGTTCAACAAGGTGACCCTGGCCGACGCCGGCTTCATCAAGC | |
| | AGTACGGCGACTGCCTGGGCGACATCGCCGCCAGGGACCTGATCTG | |
| | CGCCCAGAAGTTCAACGGCCTGACCGTGCTGCCCCCCCTGCTGACC | |
| | GACGAGATGATCGCCCAGTACACCAGCGCCCTGCTGGCCGGCACCA | |
| | TCACCAGCGGCTGGACCTTCGGCGCCGGCCCCGCCCTGCAGATCC | |
| | CCTTCCCCATGCAGATGGCCTACAGGTTCAACGGCATCGGCGTGAC | |
| | CCAGAACGTGCTGTACGAGAACCAGAAGCTGATCGCCAACCAGTTCA | |
| | ACAGCGCCATCGGCAAGATCCAGGACAGCCTGAGCAGCACCCCCAG | |
| | CGCCCTGGGCAAGCTGCAGGACGTGGTGAACCAGAACGCCCAGGC | |
| | CCTGAACACCCTGGTGAAGCAGCTGAGCAGCAACTTCGGCGCCATC | |
| | AGCAGCGTGCTGAACGACATCCTGAGCAGGCTGGACCCCCCCGAGG | |
| | CCGAGGTGCAGATCGACAGGCTGATCACCGGCAGGCTGCAGAGCCT | |
| | GCAGACCTACGTGACCCAGCAGCTGATCAGGGCCGCCGAGATCAGG | |
| | GCCAGCGCCAACCTGGCCGCCACCAAGATGAGCGAGTGCGTGCTG | |
| | GGCCAGAGCAAGAGGGTGGACTTCTGCGGCAAGGGCTACCACCTGA | |
| | TGAGCTTCCCCCAGAGCGCCCCCCACGGCGTGGTGTTCCTGCACGT | |
| | GACCTACGTGCCCGCCCAGGAGAAGAACTTCACCACCGCCCCCGCC | |
| | ATCTGCCACGACGGCAAGGCCCACTTCCCCAGGGAGGGCGTGTTCG | |
| | TGAGCAACGGCACCCACTGGTTCGTGACCCAGAGGAACTTCTACGA | |
| | GCCCCAGATCATCACCACCGACAACACCTTCGTGAGCGGCAACTGC | |
| | GACGTGGTGATCGGCATCGTGAACAACACCGTGTACGACCCCCTGC | |
| | AGCCCGAGCTGGACAGCTTCAAGGAGGAGCTGGACAAGTACTTCAA | |
| | GAACCACACCAGCCCCGACGTGGACCTGGGCGACATCAGCGGCATC | |
| | AACGCCAGCGTGGTGAACATCCAGAAGGAGATCGACAGGCTGAACG | |
| | AGGTGGCCAAGAACCTGAACGAGAGCCTGATCGACCTGCAGGAGCT | |
| | GGGCAAGTACGAGCAGTACATCAAGTGGCCCTGGTACATCTGGCTG | |
| | GGCTTCATCGCCGGCCTGATCGCCATCGTGATGGTGACCATCATGCT | |
| | GTGCTGCATGACCAGCTGCTGCAGCTGCCTGAAGGGCTGCTGCAGC | |
| | TGCGGCAGCTGCTGCAAGTTCGACGAGGACGACAGCGAGCCCGTG | |
| | CTGAAGGGCGTGAAGCTGCACTACACCGGAAGCGGAGCCACGAACT | |
| | TCTCTCTGTTAAAGCAAGCAGGAGATGTTGAAGAAAACCCCGGGCCT | |
| | ATGAAGGCAATACTAGTAGTTCTGCTATATACATTTGCAACCGCAAAT | |
| | GCAGACACATTATGTATAGGTTATCATGCGAACAACTCAACAGACACT | |
| | GTAGACACAGTACTAGAAAAGAATGTAACAGTAACACACTCTGTTAAC | |
| | CTTCTAGAAGACAAGCATAACGGGAAACTATGCAAACTAAGAGGGGT | |
| | AGCCCCATTGCATTTGGGTAAATGTAACATTGCTGGCTGGATCCTGG | |
| | GAAATCCAGAGTGTGAATCACTCTCCACAGCAAGCTCATGGTCCTAC | |
| | ATTGTGGAAACATCTAGTTCAGACAATGGAACGTGTTACCCAGGAGA | |
| | TTTCATCGATTATGAGGAGCTAAGAGAGCAATTGAGCTCAGTGTCAT | |
| | CATTTGAAAGGTTTGAGATATTCCCCAAGACAAGTTCATGGCCCAATC | |
| | ATGACTCGAACAAAGGTGTAACGACAGCATGTCCTCATGCTGGAGCA | |
| | AAAAGCTTCTACAAAAATTTAATATGGCTAGTTAAAAAAGGAAATTCAT | |
| | ACCCAAAGCTCAGCAAATCCTACATTAATGATAAAGGGAAAGAAGTC | |
| | CTCGTGCTATGGGGCATTCACCATCCACCTACTAGTGCTGACCAACA | |
| | AAGTCTCTATCAGAATGCAGATGCATATGTTTTTGTGGGGACATCAAG | |
| | ATACAGCAAGAAGTTCAAGCCGGAAATAGCAATAAGGCCCAAAGTGA | |
| | GGGATCAAGAAGGGAGAATGAACTATTACTGGACACTAGTAGAGCCG | |
| | GGAGACAAAATAACATTCGAAGCAACTGGAAATCTAGTGGTACCGAG | |
| | ATATGCATTCGCAATGGAAAGAAATGCTGGATCTGGTATTATCATTTC | |
| | AGATACACCAGTCCACGATTGCAATACAACTTGTCAGACACCCAAGG | |
| | GTGCTATAAACACCAGCCTCCCATTTCAGAACATACATCCGATCACAA | |
| | TTGGAAAATGTCCAAAATATGTAAAAAGCACAAAATTGAGACTGGCCA | |
| | CAGGATTGAGGAATGTCCCGTCCATTCAATCTAGAGGCCTATTTGGG | |
| | GCCATTGCCGGTTTCATTGAAGGGGGGTGGACAGGGATGGTAGATG | |
| | GATGGTACGGTTATCACCATCAAAATGAGCAGGGGTCAGGATATGCA | |
| | GCCGACCTGAAGAGCACACAGAATGCCATTGACGAGATTACTAACAA | |
| | AGTAAACTCTGTTATTGAAAAAATGAATACACAGTTCACAGCAGTAGG | |
| | TAAAGAGTTCAACCACCTGGAAAAAAGAATAGAGAATTTAAATAAAAA | |
| | AGTTGATGATGGTTTCCTGGACATTTGGACTTACAATGCCGAACTGTT | |
| | GGTTCTATTGGAAAATGAAAGAACTTTGGACTACCACGATTCAAAGGT | |
| | GAAGAACTTATATGAAAAGGTAAGAAGCCAGTTAAAAAACAATGCCAA | |
| | GGAAATTGGAAACGGCTGCTTTGAATTTTACCACAAATGTGATAACAC | |
| | GTGCATGGAAAGTGTCAAAAATGGGACTTATGACTACCCAAAATACT | |
| | CAGAGGAAGCAAAATTAAACAGAGAAGAAATAGATGGGGTAAAGCTG | |
| | GAATCAACAAGGATTTACCAGATTTTGGCGATCTATTCAACCGTCGCC | |
| | AGTTCATTGGTACTGGTAGTCTCCCTGGGGGCAATCAGTTTCTGGAT | |
| | GTGCTCTAATGGGTCTCTACAGTGTAGAATATGTATTTAAGGAAGCG | |
| | GAGCCACGAACTTCTCTCTGTTAAAGCAAGCAGGAGATGTTGAAGAA | |
| | AACCCCGGGCCTAGCAAAAGCAGGGGATAATTCTATTAACCATGAAG | |
| | ACTATCATTGCTTTGAGCTACATTCTATGTCTGGTTTTCGCTCAAAAA | |
| | CTTCCTGGAAATGACAATAGCACTGCAACGCTGTGCCTTGGGCACCA | |
| | TGCAGTACCAAACGGAACGATAGTGAAAACAATCACGAATGACCAAA | |
| | TTGAAGTTACTAATGCTACTGAGCTGGTTCAGAATTCCTCAATAGGTG | |
| | AAATATGCGACAGTCCTCATCAGATCCTTGATGGAGAAAACTGCACA | |
| | CTAATAGATGCTCTATTGGGAGACCCTCAGTGTGATGGCTTTCAAAAT | |
| | AAGAAATGGGACCTTTTTGTTGAACGAAGCAAAGCCTACAGCAACTG | |
| | TTACCCTTATGATGTGCCGGATTATGCCTCCCTTAGGTCACTAGTTGC | |
| | CTCATCCGGCACACTGGAGTTTAACAATGAAAGCTTCAATTGGGCTG | |
| | GAGTCACTCAAAACGGAACAAGTTCTGCTTGCATAAGGGGATCTAAT | |
| | AGTAGTTTCTTTAGTAGATTAAATTGGTTGACCCACTTAAACTTCAAGT | |
| | ACCCAGCATTGAACGTGACTATGCCAAACAATGAACAATTTGACAAAT | |
| | TGTACATTTGGGGGGTTCACCACCCGGGTACGGACAAGGACCAAAT | |
| | CTTCCTGTATGCTCAATCATCAGGAAGAATCACAGTATCTACCAAAAG | |
| | AAGCCAACAAGCTGTAATCCCGAATATCGGATCTAGACCCAGAATAA | |
| | GGAATATCCCTAGCAGAATAAGCATCTATTGGACAATAGTAAAACCG | |
| | GGAGACATACTTTTGATTAACAGCACAGGGAATCTAATTGCTCCTAG | |
| | GGGTTACTTCAAAATACGAAGTGGGAAAAGCTCAATAATGAGATCAG | |
| | ATGCACCCATTGGCAAATGCAAGTCTGAATGCATCACTCCAAATGGA | |
| | AGCATTCCCAATGACAAACCATTCCAAAATGTAAACAGGATCACATAC | |
| | GGGGCCTGTCCCAGATATGTTAAGCAAAGCACTCTGAAATTGGCAAC | |
| | AGGAATGCGAAATGTACCAGAGAAACAAACTAGAGGCATATTTGGCG | |
| | CAATAGCGGGTTTCATAGAAAATGGTTGGGAGGGAATGGTGGATGGT | |
| | TGGTACGGTTTCAGGCATCAAAATTCTGAGGGAAGAGGACAAGCAGC | |
| | AGATCTCAAAAGCACTCAAGCAGCAATCGATCAAATCAATGGGAAGC | |
| | TGAATCGATTGATCGGGAAAACCAACGAGAAATTCCATCAGATTGAG | |
| | AAAGAATTCTCAGAAGTAGAAGGGAGAATTCAGGACCTTGAGAAATA | |
| | TGTTGAGGACACAAAAATAGATCTCTGGTCATACAACGCAGAGCTTC | |
| | TTGTTGCCCTGGAAAACCAACATACAATTGATCTAACTGACTCAGAAA | |
| | TGAACAAACTGTTTGAAAAAACAAAGAAGCAACTGAGGGAAAATGCT | |
| | GAGGATATGGGCAATGGTTGTTTCAAAATATACCACAAATGTGACAAT | |
| | GCCTGCATAGGATCAATCAGAAATGGAACTTATGACCACGATGTATA | |
| | CAGGGATGAAGCATTAAACAACCGGTTCCAGATCAAGGGAGTTGAGC | |
| | TGAAGTCAGGGTACAAAGATTGGATCCTATGGATTTCCTTTGCCATAT | |
| | CATGTTTTTTGCTTTGTGTTGCTTTGTTGGGGTTCATCATGTGGGCCT | |
| | GCCAAAAGGGCAACATTAGGTGCAACATTTGCATTTGAGTGCATTAAT | |
| | TAAAAACACCCTTGTTTCTACTGGAAGCGGAGCCACGAACTTCTCTCT | |
| | GTTAAAGCAAGCAGGAGATGTTGAAGAAAACCCCGGGCCTATTTTCT | |
| | AATATCCACAAAATGAAGGCAATAATTGTACTACTCATGGTAGTAACA | |
| | TCCAATGCAGATCGAATCTGCACTGGGATAACATCGTCAAACTCACC | |
| | ACATGTCGTCAAAACTGCTACTCAAGGGGAGGTCAACGTGACCGGT | |
| | GTAATACCACTGACAACAACACCCACCAAATCTCATTTTGCAAATCTC | |
| | AAAGGAACAGAAACCAGGGGGAAACTATGCCCAAAATGCCTCAACTG | |
| | CACAGATCTGGATGTAGCCTTGGGCAGACCAAAATGCACAGGGAAAA | |
| | TACCCTCTGCAAGGGTTTCAATACTCCATGAAGTCAGACCTGTTACAT | |
| | CTGGGTGCTTTCCTATAATGCACGATAGAACAAAAATTAGACAGCTG | |
| | CCTAACCTTCTCCGAGGATACGAACATGTCAGGTTATCAACTCACAA | |
| | CGTTATCAATGCAAAAGATGCACCAGGAAGACCCTACGAAATTGGAA | |
| | CCTCAGGGTCTTGCCCTAACATTACCAATGGAAACGGATTCTTCGCA | |
| | ACAATGGCTTGGGCCGTCCCAAAAAACAAAACAGCAACAAATCCATT | |
| | AACAATAGAAGTACCATACATTTTACAGAAGGAGAAGACCAAATTAC | |
| | CGTTTGGGGGTTCCACTCTGACAACGAGACCCAAATGGCAAAGCTCT | |
| | ATGGGGACTCAAAGCCCCAGAAGTTCACCTCATCTGCCAACGGAGT | |
| | GACCACACATTACGTTTCACAGATTGGTGGCTTCCCAAATCAAACAG | |
| | AAGACGGAGGACTACCACAAAGTGGCAGAATTGTTGTTGATTACATG | |
| | GTGCAGAAATCTGGAAAAACAGGAACAATTACCTATCAAAGAGGTATT | |
| | TTATTGCCTCAAAAAGTGTGGTGCGCAAGTGGCAGGAGCAAGGTAAT | |
| | AAAAGGATCCTTGCCCTTAATTGGAGAAGCAGATTGCCTCCATGAAA | |
| | AATACGGTGGATTAAACAAAAGCAAGCCTTACTACACAGGGGAACAT | |
| | GCAAAGGCCATAGGAAATTGCCCAATATGGGTGAAAACACCCTTGAA | |
| | GCTGGCCAATGGAACCAAATATAGACCCCCTGCAAAACTATTAAAGG | |
| | AAAGAGGTTTCTTCGGAGCCATTGCTGGTTTCTTAGAGGGAGGATGG | |
| | GAAGGAATGATTGCAGGTTGGCACGGATACACATCCCATGGGGCAC | |
| | ATGGAGTAGCGGTGGCAGCTGACCTTAAGAGCACTCAAGAGGCCAT | |
| | AAACAAGATAACAAAAAATCTCAACTCTTTGAGTGAGCTGGAAGTAAA | |
| | GAATCTTCAAAGACTAAGCGGTGCCATGGATGAACTCCACAACGAAA | |
| | TACTAGAACTAGATGAGAAAGTGGATGATCTCAGAGCTGACACAATA | |
| | AGCTCACAAATAGAACTCGCAGTCCTGCTTTCCAATGAAGGAATAATA | |
| | AACAGTGAAGATGAACATCTCTTGGCGCTTGAAAGAAAGCTGAAGAA | |
| | AATGCTGGGCCCCTCTGCTGTAGAGATAGGGAATGGATGCTTTGAAA | |
| | CCAAACACAAGTGCAACCAGACCTGCCTCGACAGAATAGCTGCTGGT | |
| | ACCTTTGATGCAGGAGAATTTTCTCTCCCCACCTTTGATTCACTGAAT | |
| | ATTACTGCTGCATCTTTAAATGACGACGGATTGGACAATCATACTATA | |
| | CTGCTTTACTACTCAACTGCTGCCTCCAGTTTGGCTGTAACACTGATG | |
| | ATAGCTATCTTTGTTGTTTATATGGTCTCCAGAGACAATGTTTCTTGCT | |
| | CCATTTGTCTATAAGGAAAGTTAAGCCCTGTATTTTCCTTTATTGTAGT | |
| | GCTTGTTTGCTTGTTGTCATTACAAAGAAACGTTATTGAAAAATGGAA | |
| | GCGGAGCCACGAACTTCTCTCTGTTAAAGCAAGCAGGAGATGTTGAA | |
| | GAAAACCCCGGGCCTTTCGGAGCTATTGCTGGTTTCTTGGAAGGAG | |
| | GATGGGAAGGAATGATTGCAGGTTGGCACGGATACACATCTCATGGA | |
| | GCACATGGAGTAGCAGTGGCAGCAGACCTTAAGAGTACCCATGATG | |
| | ACTCGAGCTGGTACTGCATGCACGCAATGCTAGCTGCCCCTTTCCCG | |
| | TCCTGGGTACCCCGAGTCTCCCCCGACCTCGGGTCCCAGGTATGCT | |
| | CCCACCTCCACCTGCCCCACTCACCACCTCTGCTAGTTCCAGACACC | |
| | TCCCAAGCACGCAGCAATGCAGCTCAAAACGCTTAGCCTAGCCACAC | |
| | CCCCACGGGAAACAGCAGTGATTAACCTTTAGCAATAAACGAAAGTT | |
| | TAACTAAGCTATACTAACCCCAGGGTTGGTCAATTTCGTGCCAGCCA | |
| | CACCCTGGAGCTAGCAAAAAAAA | |
| Pan-CoV-Flu | TAGTTATTAATAGTAATCAATTACGGGGTCATTAGTTCATAGCCCATAT | 400 |
| Vaccine #12 | ATGGAGTTCCGCGTTACATAACTTACGGTAAATGGCCCGCCTGGCTG | |
| CoV-Wuhan | ACCGCCCAACGACCCCCGCCCATTGACGTCAATAATGACGTATGTTC | |
| Spike(2 proline | CCATAGTAACGCCAATAGGGACTTTCCATTGACGTCAATGGGTGGAG | |
| substitution)- | TATTTACGGTAAACTGCCCACTTGGCAGTACATCAAGTGTATCATATG | |
| influenza (HA) | CCAAGTACGCCCCCTATTGACGTCAATGACGGTAAATGGCCCGCCTG | |
| | GCATTATGCCCAGTACATGACCTTATGGGACTTTCCTACTTGGCAGTA | |
| | CATCTACGTATTAGTCATCGCTATTACCATGGTGATGCGGTTTTGGCA | |
| | GTACATCAATGGGCGTGGATAGCGGTTTGACTCACGGGGATTTCCAA | |
| | GTCTCCACCCCATTGACGTCAATGGGAGTTTGTTTTGGCACCAAAAT | |
| | CAACGGGACTTTCCAAAATGTCGTAACAACTCCGCCCCATTGACGCA | |
| | AATGGGCGGTAGGCGTGTACGGTGGGAGGTCTATATAAGCAGAGCT | |
| | GGTTTAGTGAACCGTCAGATCGAGAATAAACTAGTATTCTTCTGGTCC | |
| | CCACAGACTCAGAGAGAACCCGCCACCATGTTCGTGTTCCTGGTGCT | |
| | GCTGCCCCTGGTGAGCAGCCAGTGCGTGAACCTGACCACCAGGACC | |
| | CAGCTGCCCCCCGCCTACACCAACAGCTTCACCAGGGGCGTGTACT | |
| | ACCCCGACAAGGTGTTCAGGAGCAGCGTGCTGCACAGCACCCAGGA | |
| | CCTGTTCCTGCCCTTCTTCAGCAACGTGACCTGGTTCCACGCCATCC | |
| | ACGTGAGCGGCACCAACGGCACCAAGAGGTTCGACAACCCCGTGCT | |
| | GCCCTTCAACGACGGCGTGTACTTCGCCAGCACCGAGAAGAGCAAC | |
| | ATCATCAGGGGCTGGATCTTCGGCACCACCCTGGACAGCAAGACCC | |
| | AGAGCCTGCTGATCGTGAACAACGCCACCAACGTGGTGATCAAGGT | |
| | GTGCGAGTTCCAGTTCTGCAACGACCCCTTCCTGGGCGTGTACTACC | |
| | ACAAGAACAACAAGAGCTGGATGGAGAGCGAGTTCAGGGTGTACAG | |
| | CAGCGCCAACAACTGCACCTTCGAGTACGTGAGCCAGCCCTTCCTG | |
| | ATGGACCTGGAGGGCAAGCAGGGCAACTTCAAGAACCTGAGGGAGT | |
| | TCGTGTTCAAGAACATCGACGGCTACTTCAAGATCTACAGCAAGCAC | |
| | ACCCCCATCAACCTGGTGAGGGACCTGCCCCAGGGCTTCAGCGCCC | |
| | TGGAGCCCCTGGTGGACCTGCCCATCGGCATCAACATCACCAGGTT | |
| | CCAGACCCTGCTGGCCCTGCACAGGAGCTACCTGACCCCCGGCGAC | |
| | AGCAGCAGCGGCTGGACCGCCGGCGCCGCCGCCTACTACGTGGGC | |
| | TACCTGCAGCCCAGGACCTTCCTGCTGAAGTACAACGAGAACGGCA | |
| | CCATCACCGACGCCGTGGACTGCGCCCTGGACCCCCTGAGCGAGAC | |
| | CAAGTGCACCCTGAAGAGCTTCACCGTGGAGAAGGGCATCTACCAG | |
| | ACCAGCAACTTCAGGGTGCAGCCCACCGAGAGCATCGTGAGGTTCC | |
| | CCAACATCACCAACCTGTGCCCCTTCGGCGAGGTGTTCAACGCCAC | |
| | CAGGTTCGCCAGCGTGTACGCCTGGAACAGGAAGAGGATCAGCAAC | |
| | TGCGTGGCCGACTACAGCGTGCTGTACAACAGCGCCAGCTTCAGCA | |
| | CCTTCAAGTGCTACGGCGTGAGCCCCACCAAGCTGAACGACCTGTG | |
| | CTTCACCAACGTGTACGCCGACAGCTTCGTGATCAGGGGCGACGAG | |
| | GTGAGGCAGATCGCCCCCGGCCAGACCGGCAAGATCGCCGACTACA | |
| | ACTACAAGCTGCCCGACGACTTCACCGGCTGCGTGATCGCCTGGAA | |
| | CAGCAACAACCTGGACAGCAAGGTGGGCGGCAACTACAACTACCTG | |
| | TACAGGCTGTTCAGGAAGAGCAACCTGAAGCCCTTCGAGAGGGACA | |
| | TCAGCACCGAGATCTACCAGGCCGGCAGCACCCCCTGCAACGGCGT | |
| | GGAGGGCTTCAACTGCTACTTCCCCCTGCAGAGCTACGGCTTCCAG | |
| | CCCACCAACGGCGTGGGCTACCAGCCCTACAGGGTGGTGGTGCTGA | |
| | GCTTCGAGCTGCTGCACGCCCCCGCCACCGTGTGCGGCCCCAAGAA | |
| | GAGCACCAACCTGGTGAAGAACAAGTGCGTGAACTTCAACTTCAACG | |
| | GCCTGACCGGCACCGGCGTGCTGACCGAGAGCAACAAGAAGTTCCT | |
| | GCCCTTCCAGCAGTTCGGCAGGGACATCGCCGACACCACCGACGCC | |
| | GTGAGGGACCCCCAGACCCTGGAGATCCTGGACATCACCCCCTGCA | |
| | GCTTCGGCGGCGTGAGCGTGATCACCCCCGGCACCAACACCAGCAA | |
| | CCAGGTGGCCGTGCTGTACCAGGACGTGAACTGCACCGAGGTGCCC | |
| | GTGGCCATCCACGCCGACCAGCTGACCCCCACCTGGAGGGTGTACA | |
| | GCACCGGCAGCAACGTGTTCCAGACCAGGGCCGGCTGCCTGATCG | |
| | GCGCCGAGCACGTGAACAACAGCTACGAGTGCGACATCCCCATCGG | |
| | CGCCGGCATCTGCGCCAGCTACCAGACCCAGACCAACAGCCCCAGG | |
| | AGGGCCAGGAGCGTGGCCAGCCAGAGCATCATCGCCTACACCATGA | |
| | GCCTGGGCGCCGAGAACAGCGTGGCCTACAGCAACAACAGCATCGC | |
| | CATCCCCACCAACTTCACCATCAGCGTGACCACCGAGATCCTGCCCG | |
| | TGAGCATGACCAAGACCAGCGTGGACTGCACCATGTACATCTGCGG | |
| | CGACAGCACCGAGTGCAGCAACCTGCTGCTGCAGTACGGCAGCTTC | |
| | TGCACCCAGCTGAACAGGGCCCTGACCGGCATCGCCGTGGAGCAG | |
| | GACAAGAACACCCAGGAGGTGTTCGCCCAGGTGAAGCAGATCTACA | |
| | AGACCCCCCCCATCAAGGACTTCGGCGGCTTCAACTTCAGCCAGATC | |
| | CTGCCCGACCCCAGCAAGCCCAGCAAGAGGAGCTTCATCGAGGACC | |
| | TGCTGTTCAACAAGGTGACCCTGGCCGACGCCGGCTTCATCAAGCA | |
| | GTACGGCGACTGCCTGGGCGACATCGCCGCCAGGGACCTGATCTGC | |
| | GCCCAGAAGTTCAACGGCCTGACCGTGCTGCCCCCCCTGCTGACCG | |
| | ACGAGATGATCGCCCAGTACACCAGCGCCCTGCTGGCCGGCACCAT | |
| | CACCAGCGGCTGGACCTTCGGCGCCGGCGCCGCCCTGCAGATCCC | |
| | CTTCGCCATGCAGATGGCCTACAGGTTCAACGGCATCGGCGTGACC | |
| | CAGAACGTGCTGTACGAGAACCAGAAGCTGATCGCCAACCAGTTCAA | |
| | CAGCGCCATCGGCAAGATCCAGGACAGCCTGAGCAGCACCGCCAGC | |
| | GCCCTGGGCAAGCTGCAGGACGTGGTGAACCAGAACGCCCAGGCC | |
| | CTGAACACCCTGGTGAAGCAGCTGAGCAGCAACTTCGGCGCCATCA | |
| | GCAGCGTGCTGAACGACATCCTGAGCAGGCTGGACCCCCCCGAGG | |
| | CCGAGGTGCAGATCGACAGGCTGATCACCGGCAGGCTGCAGAGCCT | |
| | GCAGACCTACGTGACCCAGCAGCTGATCAGGGCCGCCGAGATCAGG | |
| | GCCAGCGCCAACCTGGCCGCCACCAAGATGAGCGAGTGCGTGCTG | |
| | GGCCAGAGCAAGAGGGTGGACTTCTGCGGCAAGGGCTACCACCTGA | |
| | TGAGCTTCCCCCAGAGCGCCCCCCACGGCGTGGTGTTCCTGCACGT | |
| | GACCTACGTGCCCGCCCAGGAGAAGAACTTCACCACCGCCCCCGCC | |
| | ATCTGCCACGACGGCAAGGCCCACTTCCCCAGGGAGGGCGTGTTCG | |
| | TGAGCAACGGCACCCACTGGTTCGTGACCCAGAGGAACTTCTACGA | |
| | GCCCCAGATCATCACCACCGACAACACCTTCGTGAGCGGCAACTGC | |
| | GACGTGGTGATCGGCATCGTGAACAACACCGTGTACGACCCCCTGC | |
| | AGCCCGAGCTGGACAGCTTCAAGGAGGAGCTGGACAAGTACTTCAA | |
| | GAACCACACCAGCCCCGACGTGGACCTGGGCGACATCAGCGGCATC | |
| | AACGCCAGCGTGGTGAACATCCAGAAGGAGATCGACAGGCTGAACG | |
| | AGGTGGCCAAGAACCTGAACGAGAGCCTGATCGACCTGCAGGAGCT | |
| | GGGCAAGTACGAGCAGTACATCAAGTGGCCCTGGTACATCTGGCTG | |
| | GGCTTCATCGCCGGCCTGATCGCCATCGTGATGGTGACCATCATGCT | |
| | GTGCTGCATGACCAGCTGCTGCAGCTGCCTGAAGGGCTGCTGCAGC | |
| | TGCGGCAGCTGCTGCAAGTTCGACGAGGACGACAGCGAGCCCGTG | |
| | CTGAAGGGCGTGAAGCTGCACTACACCGGAAGCGGAGCCACGAACT | |
| | TCTCTCTGTTAAAGCAAGCAGGAGATGTTGAAGAAAACCCCGGGCCT | |
| | ATGAAGGCAATACTAGTAGTTCTGCTATATACATTTGCAACCGCAAAT | |
| | GCAGACACATTATGTATAGGTTATCATGCGAACAACTCAACAGACACT | |
| | GTAGACACAGTACTAGAAAAGAATGTAACAGTAACACACTCTGTTAAC | |
| | CTTCTAGAAGACAAGCATAACGGGAAACTATGCAAACTAAGAGGGGT | |
| | AGCCCCATTGCATTTGGGTAAATGTAACATTGCTGGCTGGATCCTGG | |
| | GAAATCCAGAGTGTGAATCACTCTCCACAGCAAGCTCATGGTCCTAC | |
| | ATTGTGGAAACATCTAGTTCAGACAATGGAACGTGTTACCCAGGAGA | |
| | TTTCATCGATTATGAGGAGCTAAGAGAGCAATTGAGCTCAGTGTCAT | |
| | CATTTGAAAGGTTTGAGATATTCCCCAAGACAAGTTCATGGCCCAATC | |
| | ATGACTCGAACAAAGGTGTAACGACAGCATGTCCTCATGCTGGAGCA | |
| | AAAAGCTTCTACAAAAATTTAATATGGCTAGTTAAAAAAGGAAATTCAT | |
| | ACCCAAAGCTCAGCAAATCCTACATTAATGATAAAGGGAAAGAAGTC | |
| | CTCGTGCTATGGGGCATTCACCATCCACCTACTAGTGCTGACCAACA | |
| | AAGTCTCTATCAGAATGCAGATGCATATGTTTTTGTGGGGACATCAAG | |
| | ATACAGCAAGAAGTTCAAGCCGGAAATAGCAATAAGGCCCAAAGTGA | |
| | GGGATCAAGAAGGGAGAATGAACTATTACTGGACACTAGTAGAGCCG | |
| | GGAGACAAAATAACATTCGAAGCAACTGGAAATCTAGTGGTACCGAG | |
| | ATATGCATTCGCAATGGAAAGAAATGCTGGATCTGGTATTATCATTTC | |
| | AGATACACCAGTCCACGATTGCAATACAACTTGTCAGACACCCAAGG | |
| | GTGCTATAAACACCAGCCTCCCATTTCAGAACATACATCCGATCACAA | |
| | TTGGAAAATGTCCAAAATATGTAAAAAGCACAAAATTGAGACTGGCCA | |
| | CAGGATTGAGGAATGTCCCGTCCATTCAATCTAGAGGCCTATTTGGG | |
| | GCCATTGCCGGTTTCATTGAAGGGGGGTGGACAGGGATGGTAGATG | |
| | GATGGTACGGTTATCACCATCAAAATGAGCAGGGGTCAGGATATGCA | |
| | GCCGACCTGAAGAGCACACAGAATGCCATTGACGAGATTACTAACAA | |
| | AGTAAACTCTGTTATTGAAAAAATGAATACACAGTTCACAGCAGTAGG | |
| | TAAAGAGTTCAACCACCTGGAAAAAAGAATAGAGAATTTAAATAAAAA | |
| | AGTTGATGATGGTTTCCTGGACATTTGGACTTACAATGCCGAACTGTT | |
| | GGTTCTATTGGAAAATGAAAGAACTTTGGACTACCACGATTCAAAGGT | |
| | GAAGAACTTATATGAAAAGGTAAGAAGCCAGTTAAAAAACAATGCCAA | |
| | GGAAATTGGAAACGGCTGCTTTGAATTTTACCACAAATGTGATAACAC | |
| | GTGCATGGAAAGTGTCAAAAATGGGACTTATGACTACCCAAAATACT | |
| | CAGAGGAAGCAAAATTAAACAGAGAAGAAATAGATGGGGTAAAGCTG | |
| | GAATCAACAAGGATTTACCAGATTTTGGCGATCTATTCAACCGTCGCC | |
| | AGTTCATTGGTACTGGTAGTCTCCCTGGGGGCAATCAGTTTCTGGAT | |
| | GTGCTCTAATGGGTCTCTACAGTGTAGAATATGTATTTAAGGAAGCG | |
| | GAGCCACGAACTTCTCTCTGTTAAAGCAAGCAGGAGATGTTGAAGAA | |
| | AACCCCGGGCCTAGCAAAAGCAGGGGATAATTCTATTAACCATGAAG | |
| | ACTATCATTGCTTTGAGCTACATTCTATGTCTGGTTTTCGCTCAAAAA | |
| | CTTCCTGGAAATGACAATAGCACTGCAACGCTGTGCCTTGGGCACCA | |
| | TGCAGTACCAAACGGAACGATAGTGAAAACAATCACGAATGACCAAA | |
| | TTGAAGTTACTAATGCTACTGAGCTGGTTCAGAATTCCTCAATAGGTG | |
| | AAATATGCGACAGTCCTCATCAGATCCTTGATGGAGAAAACTGCACA | |
| | CTAATAGATGCTCTATTGGGAGACCCTCAGTGTGATGGCTTTCAAAAT | |
| | AAGAAATGGGACCTTTTTGTTGAACGAAGCAAAGCCTACAGCAACTG | |
| | TTACCCTTATGATGTGCCGGATTATGCCTCCCTTAGGTCACTAGTTGC | |
| | CTCATCCGGCACACTGGAGTTTAACAATGAAAGCTTCAATTGGGCTG | |
| | GAGTCACTCAAAACGGAACAAGTTCTGCTTGCATAAGGGGATCTAAT | |
| | AGTAGTTTCTTTAGTAGATTAAATTGGTTGACCCACTTAAACTTCAAGT | |
| | ACCCAGCATTGAACGTGACTATGCCAAACAATGAACAATTTGACAAAT | |
| | TGTACATTTGGGGGGTTCACCACCCGGGTACGGACAAGGACCAAAT | |
| | CTTCCTGTATGCTCAATCATCAGGAAGAATCACAGTATCTACCAAAAG | |
| | AAGCCAACAAGCTGTAATCCCGAATATCGGATCTAGACCCAGAATAA | |
| | GGAATATCCCTAGCAGAATAAGCATCTATTGGACAATAGTAAAACCG | |
| | GGAGACATACTTTTGATTAACAGCACAGGGAATCTAATTGCTCCTAG | |
| | GGGTTACTTCAAAATACGAAGTGGGAAAAGCTCAATAATGAGATCAG | |
| | ATGCACCCATTGGCAAATGCAAGTCTGAATGCATCACTCCAAATGGA | |
| | AGCATTCCCAATGACAAACCATTCCAAAATGTAAACAGGATCACATAC | |
| | GGGGCCTGTCCCAGATATGTTAAGCAAAGCACTCTGAAATTGGCAAC | |
| | AGGAATGCGAAATGTACCAGAGAAACAAACTAGAGGCATATTTGGCG | |
| | CAATAGCGGGTTTCATAGAAAATGGTTGGGAGGGAATGGTGGATGGT | |
| | TGGTACGGTTTCAGGCATCAAAATTCTGAGGGAAGAGGACAAGCAGC | |
| | AGATCTCAAAAGCACTCAAGCAGCAATCGATCAAATCAATGGGAAGC | |
| | TGAATCGATTGATCGGGAAAACCAACGAGAAATTCCATCAGATTGAG | |
| | AAAGAATTCTCAGAAGTAGAAGGGAGAATTCAGGACCTTGAGAAATA | |
| | TGTTGAGGACACAAAAATAGATCTCTGGTCATACAACGCAGAGCTTC | |
| | TTGTTGCCCTGGAAAACCAACATACAATTGATCTAACTGACTCAGAAA | |
| | TGAACAAACTGTTTGAAAAAACAAAGAAGCAACTGAGGGAAAATGCT | |
| | GAGGATATGGGCAATGGTTGTTTCAAAATATACCACAAATGTGACAAT | |
| | GCCTGCATAGGATCAATCAGAAATGGAACTTATGACCACGATGTATA | |
| | CAGGGATGAAGCATTAAACAACCGGTTCCAGATCAAGGGAGTTGAGC | |
| | TGAAGTCAGGGTACAAAGATTGGATCCTATGGATTTCCTTTGCCATAT | |
| | CATGTTTTTTGCTTTGTGTTGCTTTGTTGGGGTTCATCATGTGGGCCT | |
| | GCCAAAAGGGCAACATTAGGTGCAACATTTGCATTTGAGTGCATTAAT | |
| | TAAAAACACCCTTGTTTCTACTGGAAGCGGAGCCACGAACTTCTCTCT | |
| | GTTAAAGCAAGCAGGAGATGTTGAAGAAAACCCCGGGCCTATTTTCT | |
| | AATATCCACAAAATGAAGGCAATAATTGTACTACTCATGGTAGTAACA | |
| | TCCAATGCAGATCGAATCTGCACTGGGATAACATCGTCAAACTCACC | |
| | ACATGTCGTCAAAACTGCTACTCAAGGGGAGGTCAACGTGACCGGT | |
| | GTAATACCACTGACAACAACACCCACCAAATCTCATTTTGCAAATCTC | |
| | AAAGGAACAGAAACCAGGGGGAAACTATGCCCAAAATGCCTCAACTG | |
| | CACAGATCTGGATGTAGCCTTGGGCAGACCAAAATGCACAGGGAAAA | |
| | TACCCTCTGCAAGGGTTTCAATACTCCATGAAGTCAGACCTGTTACAT | |
| | CTGGGTGCTTTCCTATAATGCACGATAGAACAAAAATTAGACAGCTG | |
| | CCTAACCTTCTCCGAGGATACGAACATGTCAGGTTATCAACTCACAA | |
| | CGTTATCAATGCAAAAGATGCACCAGGAAGACCCTACGAAATTGGAA | |
| | CCTCAGGGTCTTGCCCTAACATTACCAATGGAAACGGATTCTTCGCA | |
| | ACAATGGCTTGGGCCGTCCCAAAAAACAAAACAGCAACAAATCCATT | |
| | AACAATAGAAGTACCATACATTTGTACAGAAGGAGAAGACCAAATTAC | |
| | CGTTTGGGGGTTCCACTCTGACAACGAGACCCAAATGGCAAAGCTCT | |
| | ATGGGGACTCAAAGCCCCAGAAGTTCACCTCATCTGCCAACGGAGT | |
| | GACCACACATTACGTTTCACAGATTGGTGGCTTCCCAAATCAAACAG | |
| | AAGACGGAGGACTACCACAAAGTGGCAGAATTGTTGTTGATTACATG | |
| | GTGCAGAAATCTGGAAAAACAGGAACAATTACCTATCAAAGAGGTATT | |
| | TTATTGCCTCAAAAAGTGTGGTGCGCAAGTGGCAGGAGCAAGGTAAT | |
| | AAAAGGATCCTTGCCCTTAATTGGAGAAGCAGATTGCCTCCATGAAA | |
| | AATACGGTGGATTAAACAAAAGCAAGCCTTACTACACAGGGGAACAT | |
| | GCAAAGGCCATAGGAAATTGCCCAATATGGGTGAAAACACCCTTGAA | |
| | GCTGGCCAATGGAACCAAATATAGACCCCCTGCAAAACTATTAAAGG | |
| | AAAGAGGTTTCTTCGGAGCCATTGCTGGTTTCTTAGAGGGAGGATGG | |
| | GAAGGAATGATTGCAGGTTGGCACGGATACACATCCCATGGGGCAC | |
| | ATGGAGTAGCGGTGGCAGCTGACCTTAAGAGCACTCAAGAGGCCAT | |
| | AAACAAGATAACAAAAAATCTCAACTCTTTGAGTGAGCTGGAAGTAAA | |
| | GAATCTTCAAAGACTAAGCGGTGCCATGGATGAACTCCACAACGAAA | |
| | TACTAGAACTAGATGAGAAAGTGGATGATCTCAGAGCTGACACAATA | |
| | AGCTCACAAATAGAACTCGCAGTCCTGCTTTCCAATGAAGGAATAATA | |
| | AACAGTGAAGATGAACATCTCTTGGCGCTTGAAAGAAAGCTGAAGAA | |
| | AATGCTGGGCCCCTCTGCTGTAGAGATAGGGAATGGATGCTTTGAAA | |
| | CCAAACACAAGTGCAACCAGACCTGCCTCGACAGAATAGCTGCTGGT | |
| | ACCTTTGATGCAGGAGAATTTTCTCTCCCCACCTTTGATTCACTGAAT | |
| | ATTACTGCTGCATCTTTAAATGACGACGGATTGGACAATCATACTATA | |
| | CTGCTTTACTACTCAACTGCTGCCTCCAGTTTGGCTGTAACACTGATG | |
| | ATAGCTATCTTTGTTGTTTATATGGTCTCCAGAGACAATGTTTCTTGCT | |
| | CCATTTGTCTATAAGGAAAGTTAAGCCCTGTATTTTCCTTTATTGTAGT | |
| | GCTTGTTTGCTTGTTGTCATTACAAAGAAACGTTATTGAAAAATGGAA | |
| | GCGGAGCCACGAACTTCTCTCTGTTAAAGCAAGCAGGAGATGTTGAA | |
| | GAAAACCCCGGGCCTTTCGGAGCTATTGCTGGTTTCTTGGAAGGAG | |
| | GATGGGAAGGAATGATTGCAGGTTGGCACGGATACACATCTCATGGA | |
| | GCACATGGAGTAGCAGTGGCAGCAGACCTTAAGAGTACCCATGATG | |
| | ACTCGAGCTGGTACTGCATGCACGCAATGCTAGCTGCCCCTTTCCCG | |
| | TCCTGGGTACCCCGAGTCTCCCCCGACCTCGGGTCCCAGGTATGCT | |
| | CCCACCTCCACCTGCCCCACTCACCACCTCTGCTAGTTCCAGACACC | |
| | TCCCAAGCACGCAGCAATGCAGCTCAAAACGCTTAGCCTAGCCACAC | |
| | CCCCACGGGAAACAGCAGTGATTAACCTTTAGCAATAAACGAAAGTT | |
| | TAACTAAGCTATACTAACCCCAGGGTTGGTCAATTTCGTGCCAGCCA | |
| | CACCCTGGAGCTAGCAAAAAAAA | |
| Pan-CoV-Flu | CTCGACATTGATTATTGACTAGTTATTAATAGTAATCAATTACGGGGT | 401 |
| Vaccine #13 | CATTAGTTCATAGCCCATATATGGAGTTCCGCGTTACATAACTTACGG | |
| CoV-Wuhan | TAAATGGCCCGCCTGGCTGACCGCCCAACGACCCCCGCCCATTGAC | |
| Spike(HexaPro)- | GTCAATAATGACGTATGTTCCCATAGTAACGCCAATAGGGACTTTCCA | |
| Influenza (HA) | TTGACGTCAATGGGTGGAGTATTTACGGTAAACTGCCCACTTGGCAG | |
| | TACATCAAGTGTATCATATGCCAAGTACGCCCCCTATTGACGTCAATG | |
| | ACGGTAAATGGCCCGCCTGGCATTATGCCCAGTACATGACCTTATGG | |
| | GACTTTCCTACTTGGCAGTACATCTACGTATTAGTCATCGCTATTACC | |
| | ATGGTCGAGGTGAGCCCCACGTTCTGCTTCACTCTCCCCATCTCCCC | |
| | CCCCTCCCCACCCCCAATTTTGTATTTATTTATTTTTTAATTATTTTGT | |
| | GCAGCGATGGGGGCGGGGGGGGGGGGGGGGCGCGCGCCAGGCG | |
| | GGGCGGGGCGGGGCGAGGGGCGGGGCGGGGCGAGGCGGAGAGG | |
| | TGCGGCGGCAGCCAATCAGAGCGGCGCGCTCCGAAAGTTTCCTTTT | |
| | ATGGCGAGGCGGCGGCGGCGGCGGCCCTATAAAAAGCGAAGCGCG | |
| | CGGCGGGCGGGAGTCGCTGCGCGCTGCCTTCGCCCCGTGCCCCGC | |
| | TCCGCCGCCGCCTCGCGCCGCCCGCCCCGGCTCTGACTGACCGCG | |
| | TTACTCCCACAGGTGAGCGGGCGGGACGGCCCTTCTCCTCCGGGCT | |
| | GTAATTAGCGCTTGGTTTAATGACGGCTTGTTTCTTTTCTGTGGCTGC | |
| | GTGAAAGCCTTGAGGGGCTCCGGGAGGGCCCTTTGTGCGGGGGGA | |
| | GCGGCTCGGGGGGTGCGTGCGTGTGTGTGTGCGTGGGGAGCGCCG | |
| | CGTGCGGCTCCGCGCTGCCCGGCGGCTGTGAGCGCTGCGGGCGCG | |
| | GCGCGGGGCTTTGTGCGCTCCGCAGTGTGCGCGAGGGGAGCGCGG | |
| | CCGGGGGCGGTGCCCCGCGGTGCGGGGGGGGCTGCGAGGGGAAC | |
| | AAAGGCTGCGTGCGGGGTGTGTGCGTGGGGGGGTGAGCAGGGGGT | |
| | GTGGGCGCGTCGGTCGGGCTGCAACCCCCCCTGCACCCCCCTCCC | |
| | CGAGTTGCTGAGCACGGCCCGGCTTCGGGTGCGGGGCTCCGTACG | |
| | GGGCGTGGCGCGGGGCTCGCCGTGCCGGGCGGGGGGTGGCGGCA | |
| | GGTGGGGGTGCCGGGCGGGGCGGGGCCGCCTCGGGCCGGGGAG | |
| | GGCTCGGGGGAGGGGCGCGGCGGCCCCCGGAGCGCCGGCGGCTG | |
| | TCGAGGCGCGGCGAGCCGCAGCCATTGCCTTTTATGGTAATCGTGC | |
| | GAGAGGGCGCAGGGACTTCCTTTGTCCCAAATCTGTGCGGAGCCGA | |
| | AATCTGGGAGGCGCCGCCGCACCCCCTCTAGCGGGCGCGGGGCGA | |
| | AGCGGTGCGGCGCCGGCAGGAAGGAAATGGGCGGGGAGGGCCTTC | |
| | GTGCGTCGCCGCGCCGCCGTCCCCTTCTCCCTCTCCAGCCTCGGGG | |
| | CTGTCCGCGGGGGGACGGCTGCCTTCGGGGGGGACGGGGCAGGG | |
| | CGGGGTTCGGCTTCTGGCGTGTGACCGGCGGCTCTAGAGCCTCTGC | |
| | TAACCATGTTCATGCCTTCTTCTTTTTCCTACAGCTCCTGGGCAACGT | |
| | GCTGGTTATTGTGCTGTCTCATCATTTTGGCAAAGAATTGGAGAATAA | |
| | ACTAGTATTCTTCTGGTCCCCACAGACTCAGAGAGAACCCGCCACCA | |
| | TGTTCGTGTTCCTGGTGCTGCTGCCCCTGGTGAGCAGCCAGTGCGT | |
| | GAACCTGACCACCAGGACCCAGCTGCCCCCCGCCTACACCAACAGC | |
| | TTCACCAGGGGCGTGTACTACCCCGACAAGGTGTTCAGGAGCAGCG | |
| | TGCTGCACAGCACCCAGGACCTGTTCCTGCCCTTCTTCAGCAACGTG | |
| | ACCTGGTTCCACGCCATCCACGTGAGCGGCACCAACGGCACCAAGA | |
| | GGTTCGACAACCCCGTGCTGCCCTTCAACGACGGCGTGTACTTCGC | |
| | CAGCACCGAGAAGAGCAACATCATCAGGGGCTGGATCTTCGGCACC | |
| | ACCCTGGACAGCAAGACCCAGAGCCTGCTGATCGTGAACAACGCCA | |
| | CCAACGTGGTGATCAAGGTGTGCGAGTTCCAGTTCTGCAACGACCC | |
| | CTTCCTGGGCGTGTACTACCACAAGAACAACAAGAGCTGGATGGAGA | |
| | GCGAGTTCAGGGTGTACAGCAGCGCCAACAACTGCACCTTCGAGTA | |
| | CGTGAGCCAGCCCTTCCTGATGGACCTGGAGGGCAAGCAGGGCAAC | |
| | TTCAAGAACCTGAGGGAGTTCGTGTTCAAGAACATCGACGGCTACTT | |
| | CAAGATCTACAGCAAGCACACCCCCATCAACCTGGTGAGGGACCTG | |
| | CCCCAGGGCTTCAGCGCCCTGGAGCCCCTGGTGGACCTGCCCATCG | |
| | GCATCAACATCACCAGGTTCCAGACCCTGCTGGCCCTGCACAGGAG | |
| | CTACCTGACCCCCGGCGACAGCAGCAGCGGCTGGACCGCCGGCGC | |
| | CGCCGCCTACTACGTGGGCTACCTGCAGCCCAGGACCTTCCTGCTG | |
| | AAGTACAACGAGAACGGCACCATCACCGACGCCGTGGACTGCGCCC | |
| | TGGACCCCCTGAGCGAGACCAAGTGCACCCTGAAGAGCTTCACCGT | |
| | GGAGAAGGGCATCTACCAGACCAGCAACTTCAGGGTGCAGCCCACC | |
| | GAGAGCATCGTGAGGTTCCCCAACATCACCAACCTGTGCCCCTTCG | |
| | GCGAGGTGTTCAACGCCACCAGGTTCGCCAGCGTGTACGCCTGGAA | |
| | CAGGAAGAGGATCAGCAACTGCGTGGCCGACTACAGCGTGCTGTAC | |
| | AACAGCGCCAGCTTCAGCACCTTCAAGTGCTACGGCGTGAGCCCCA | |
| | CCAAGCTGAACGACCTGTGCTTCACCAACGTGTACGCCGACAGCTTC | |
| | GTGATCAGGGGCGACGAGGTGAGGCAGATCGCCCCCGGCCAGACC | |
| | GGCAAGATCGCCGACTACAACTACAAGCTGCCCGACGACTTCACCG | |
| | GCTGCGTGATCGCCTGGAACAGCAACAACCTGGACAGCAAGGTGGG | |
| | CGGCAACTACAACTACCTGTACAGGCTGTTCAGGAAGAGCAACCTGA | |
| | AGCCCTTCGAGAGGGACATCAGCACCGAGATCTACCAGGCCGGCAG | |
| | CACCCCCTGCAACGGCGTGGAGGGCTTCAACTGCTACTTCCCCCTG | |
| | CAGAGCTACGGCTTCCAGCCCACCAACGGCGTGGGCTACCAGCCCT | |
| | ACAGGGTGGTGGTGCTGAGCTTCGAGCTGCTGCACGCCCCCGCCAC | |
| | CGTGTGCGGCCCCAAGAAGAGCACCAACCTGGTGAAGAACAAGTGC | |
| | GTGAACTTCAACTTCAACGGCCTGACCGGCACCGGCGTGCTGACCG | |
| | AGAGCAACAAGAAGTTCCTGCCCTTCCAGCAGTTCGGCAGGGACAT | |
| | CGCCGACACCACCGACGCCGTGAGGGACCCCCAGACCCTGGAGAT | |
| | CCTGGACATCACCCCCTGCAGCTTCGGCGGCGTGAGCGTGATCACC | |
| | CCCGGCACCAACACCAGCAACCAGGTGGCCGTGCTGTACCAGGACG | |
| | TGAACTGCACCGAGGTGCCCGTGGCCATCCACGCCGACCAGCTGAC | |
| | CCCCACCTGGAGGGTGTACAGCACCGGCAGCAACGTGTTCCAGACC | |
| | AGGGCCGGCTGCCTGATCGGCGCCGAGCACGTGAACAACAGCTAC | |
| | GAGTGCGACATCCCCATCGGCGCCGGCATCTGCGCCAGCTACCAGA | |
| | CCCAGACCAACAGCCCCGGCAGCGCCAGCAGCGTGGCCAGCCAGA | |
| | GCATCATCGCCTACACCATGAGCCTGGGCGCCGAGAACAGCGTGGC | |
| | CTACAGCAACAACAGCATCGCCATCCCCACCAACTTCACCATCAGCG | |
| | TGACCACCGAGATCCTGCCCGTGAGCATGACCAAGACCAGCGTGGA | |
| | CTGCACCATGTACATCTGCGGCGACAGCACCGAGTGCAGCAACCTG | |
| | CTGCTGCAGTACGGCAGCTTCTGCACCCAGCTGAACAGGGCCCTGA | |
| | CCGGCATCGCCGTGGAGCAGGACAAGAACACCCAGGAGGTGTTCGC | |
| | CCAGGTGAAGCAGATCTACAAGACCCCCCCCATCAAGGACTTCGGC | |
| | GGCTTCAACTTCAGCCAGATCCTGCCCGACCCCAGCAAGCCCAGCA | |
| | AGAGGAGCCCCATCGAGGACCTGCTGTTCAACAAGGTGACCCTGGC | |
| | CGACGCCGGCTTCATCAAGCAGTACGGCGACTGCCTGGGCGACATC | |
| | GCCGCCAGGGACCTGATCTGCGCCCAGAAGTTCAACGGCCTGACCG | |
| | TGCTGCCCCCCCTGCTGACCGACGAGATGATCGCCCAGTACACCAG | |
| | CGCCCTGCTGGCCGGCACCATCACCAGCGGCTGGACCTTCGGCGC | |
| | CGGCCCCGCCCTGCAGATCCCCTTCCCCATGCAGATGGCCTACAGG | |
| | TTCAACGGCATCGGCGTGACCCAGAACGTGCTGTACGAGAACCAGA | |
| | AGCTGATCGCCAACCAGTTCAACAGCGCCATCGGCAAGATCCAGGA | |
| | CAGCCTGAGCAGCACCCCCAGCGCCCTGGGCAAGCTGCAGGACGT | |
| | GGTGAACCAGAACGCCCAGGCCCTGAACACCCTGGTGAAGCAGCTG | |
| | AGCAGCAACTTCGGCGCCATCAGCAGCGTGCTGAACGACATCCTGA | |
| | GCAGGCTGGACCCCCCCGAGGCCGAGGTGCAGATCGACAGGCTGA | |
| | TCACCGGCAGGCTGCAGAGCCTGCAGACCTACGTGACCCAGCAGCT | |
| | GATCAGGGCCGCCGAGATCAGGGCCAGCGCCAACCTGGCCGCCAC | |
| | CAAGATGAGCGAGTGCGTGCTGGGCCAGAGCAAGAGGGTGGACTTC | |
| | TGCGGCAAGGGCTACCACCTGATGAGCTTCCCCCAGAGCGCCCCCC | |
| | ACGGCGTGGTGTTCCTGCACGTGACCTACGTGCCCGCCCAGGAGAA | |
| | GAACTTCACCACCGCCCCCGCCATCTGCCACGACGGCAAGGCCCAC | |
| | TTCCCCAGGGAGGGCGTGTTCGTGAGCAACGGCACCCACTGGTTCG | |
| | TGACCCAGAGGAACTTCTACGAGCCCCAGATCATCACCACCGACAAC | |
| | ACCTTCGTGAGCGGCAACTGCGACGTGGTGATCGGCATCGTGAACA | |
| | ACACCGTGTACGACCCCCTGCAGCCCGAGCTGGACAGCTTCAAGGA | |
| | GGAGCTGGACAAGTACTTCAAGAACCACACCAGCCCCGACGTGGAC | |
| | CTGGGCGACATCAGCGGCATCAACGCCAGCGTGGTGAACATCCAGA | |
| | AGGAGATCGACAGGCTGAACGAGGTGGCCAAGAACCTGAACGAGAG | |
| | CCTGATCGACCTGCAGGAGCTGGGCAAGTACGAGCAGTACATCAAG | |
| | TGGCCCTGGTACATCTGGCTGGGCTTCATCGCCGGCCTGATCGCCA | |
| | TCGTGATGGTGACCATCATGCTGTGCTGCATGACCAGCTGCTGCAGC | |
| | TGCCTGAAGGGCTGCTGCAGCTGCGGCAGCTGCTGCAAGTTCGACG | |
| | AGGACGACAGCGAGCCCGTGCTGAAGGGCGTGAAGCTGCACTACAC | |
| | CGGAAGCGGAGCCACGAACTTCTCTCTGTTAAAGCAAGCAGGAGAT | |
| | GTTGAAGAAAACCCCGGGCCTATGAAGGCAATACTAGTAGTTCTGCT | |
| | ATATACATTTGCAACCGCAAATGCAGACACATTATGTATAGGTTATCA | |
| | TGCGAACAACTCAACAGACACTGTAGACACAGTACTAGAAAAGAATG | |
| | TAACAGTAACACACTCTGTTAACCTTCTAGAAGACAAGCATAACGGGA | |
| | AACTATGCAAACTAAGAGGGGTAGCCCCATTGCATTTGGGTAAATGT | |
| | AACATTGCTGGCTGGATCCTGGGAAATCCAGAGTGTGAATCACTCTC | |
| | CACAGCAAGCTCATGGTCCTACATTGTGGAAACATCTAGTTCAGACA | |
| | ATGGAACGTGTTACCCAGGAGATTTCATCGATTATGAGGAGCTAAGA | |
| | GAGCAATTGAGCTCAGTGTCATCATTTGAAAGGTTTGAGATATTCCCC | |
| | AAGACAAGTTCATGGCCCAATCATGACTCGAACAAAGGTGTAACGAC | |
| | AGCATGTCCTCATGCTGGAGCAAAAAGCTTCTACAAAAATTTAATATG | |
| | GCTAGTTAAAAAAGGAAATTCATACCCAAAGCTCAGCAAATCCTACAT | |
| | TAATGATAAAGGGAAAGAAGTCCTCGTGCTATGGGGCATTCACCATC | |
| | CACCTACTAGTGCTGACCAACAAAGTCTCTATCAGAATGCAGATGCA | |
| | TATGTTTTTGTGGGGACATCAAGATACAGCAAGAAGTTCAAGCCGGA | |
| | AATAGCAATAAGGCCCAAAGTGAGGGATCAAGAAGGGAGAATGAACT | |
| | ATTACTGGACACTAGTAGAGCCGGGAGACAAAATAACATTCGAAGCA | |
| | ACTGGAAATCTAGTGGTACCGAGATATGCATTCGCAATGGAAAGAAA | |
| | TGCTGGATCTGGTATTATCATTTCAGATACACCAGTCCACGATTGCAA | |
| | TACAACTTGTCAGACACCCAAGGGTGCTATAAACACCAGCCTCCCAT | |
| | TTCAGAACATACATCCGATCACAATTGGAAAATGTCCAAAATATGTAA | |
| | AAAGCACAAAATTGAGACTGGCCACAGGATTGAGGAATGTCCCGTCC | |
| | ATTCAATCTAGAGGCCTATTTGGGGCCATTGCCGGTTTCATTGAAGG | |
| | GGGGTGGACAGGGATGGTAGATGGATGGTACGGTTATCACCATCAA | |
| | AATGAGCAGGGGTCAGGATATGCAGCCGACCTGAAGAGCACACAGA | |
| | ATGCCATTGACGAGATTACTAACAAAGTAAACTCTGTTATTGAAAAAA | |
| | TGAATACACAGTTCACAGCAGTAGGTAAAGAGTTCAACCACCTGGAA | |
| | AAAAGAATAGAGAATTTAAATAAAAAAGTTGATGATGGTTTCCTGGAC | |
| | ATTTGGACTTACAATGCCGAACTGTTGGTTCTATTGGAAAATGAAAGA | |
| | ACTTTGGACTACCACGATTCAAAGGTGAAGAACTTATATGAAAAGGTA | |
| | AGAAGCCAGTTAAAAAACAATGCCAAGGAAATTGGAAACGGCTGCTT | |
| | TGAATTTTACCACAAATGTGATAACACGTGCATGGAAAGTGTCAAAAA | |
| | TGGGACTTATGACTACCCAAAATACTCAGAGGAAGCAAAATTAAACA | |
| | GAGAAGAAATAGATGGGGTAAAGCTGGAATCAACAAGGATTTACCAG | |
| | ATTTTGGCGATCTATTCAACCGTCGCCAGTTCATTGGTACTGGTAGTC | |
| | TCCCTGGGGGCAATCAGTTTCTGGATGTGCTCTAATGGGTCTCTACA | |
| | GTGTAGAATATGTATTTAAGGAAGCGGAGCCACGAACTTCTCTCTGTT | |
| | AAAGCAAGCAGGAGATGTTGAAGAAAACCCCGGGCCTAGCAAAAGC | |
| | AGGGGATAATTCTATTAACCATGAAGACTATCATTGCTTTGAGCTACA | |
| | TTCTATGTCTGGTTTTCGCTCAAAAACTTCCTGGAAATGACAATAGCA | |
| | CTGCAACGCTGTGCCTTGGGCACCATGCAGTACCAAACGGAACGAT | |
| | AGTGAAAACAATCACGAATGACCAAATTGAAGTTACTAATGCTACTGA | |
| | GCTGGTTCAGAATTCCTCAATAGGTGAAATATGCGACAGTCCTCATC | |
| | AGATCCTTGATGGAGAAAACTGCACACTAATAGATGCTCTATTGGGA | |
| | GACCCTCAGTGTGATGGCTTTCAAAATAAGAAATGGGACCTTTTTGTT | |
| | GAACGAAGCAAAGCCTACAGCAACTGTTACCCTTATGATGTGCCGGA | |
| | TTATGCCTCCCTTAGGTCACTAGTTGCCTCATCCGGCACACTGGAGT | |
| | TTAACAATGAAAGCTTCAATTGGGCTGGAGTCACTCAAAACGGAACA | |
| | AGTTCTGCTTGCATAAGGGGATCTAATAGTAGTTTCTTTAGTAGATTA | |
| | AATTGGTTGACCCACTTAAACTTCAAGTACCCAGCATTGAACGTGACT | |
| | ATGCCAAACAATGAACAATTTGACAAATTGTACATTTGGGGGGTTCAC | |
| | CACCCGGGTACGGACAAGGACCAAATCTTCCTGTATGCTCAATCATC | |
| | AGGAAGAATCACAGTATCTACCAAAAGAAGCCAACAAGCTGTAATCC | |
| | CGAATATCGGATCTAGACCCAGAATAAGGAATATCCCTAGCAGAATA | |
| | AGCATCTATTGGACAATAGTAAAACCGGGAGACATACTTTTGATTAAC | |
| | AGCACAGGGAATCTAATTGCTCCTAGGGGTTACTTCAAAATACGAAG | |
| | TGGGAAAAGCTCAATAATGAGATCAGATGCACCCATTGGCAAATGCA | |
| | AGTCTGAATGCATCACTCCAAATGGAAGCATTCCCAATGACAAACCAT | |
| | TCCAAAATGTAAACAGGATCACATACGGGGCCTGTCCCAGATATGTT | |
| | AAGCAAAGCACTCTGAAATTGGCAACAGGAATGCGAAATGTACCAGA | |
| | GAAACAAACTAGAGGCATATTTGGCGCAATAGCGGGTTTCATAGAAA | |
| | ATGGTTGGGAGGGAATGGTGGATGGTTGGTACGGTTTCAGGCATCA | |
| | AAATTCTGAGGGAAGAGGACAAGCAGCAGATCTCAAAAGCACTCAAG | |
| | CAGCAATCGATCAAATCAATGGGAAGCTGAATCGATTGATCGGGAAA | |
| | ACCAACGAGAAATTCCATCAGATTGAGAAAGAATTCTCAGAAGTAGAA | |
| | GGGAGAATTCAGGACCTTGAGAAATATGTTGAGGACACAAAAATAGA | |
| | TCTCTGGTCATACAACGCAGAGCTTCTTGTTGCCCTGGAAAACCAAC | |
| | ATACAATTGATCTAACTGACTCAGAAATGAACAAACTGTTTGAAAAAA | |
| | CAAAGAAGCAACTGAGGGAAAATGCTGAGGATATGGGCAATGGTTGT | |
| | TTCAAAATATACCACAAATGTGACAATGCCTGCATAGGATCAATCAGA | |
| | AATGGAACTTATGACCACGATGTATACAGGGATGAAGCATTAAACAA | |
| | CCGGTTCCAGATCAAGGGAGTTGAGCTGAAGTCAGGGTACAAAGATT | |
| | GGATCCTATGGATTTCCTTTGCCATATCATGTTTTTTGCTTTGTGTTGC | |
| | TTTGTTGGGGTTCATCATGTGGGCCTGCCAAAAGGGCAACATTAGGT | |
| | GCAACATTTGCATTTGAGTGCATTAATTAAAAACACCCTTGTTTCTACT | |
| | GGAAGCGGAGCCACGAACTTCTCTCTGTTAAAGCAAGCAGGAGATGT | |
| | TGAAGAAAACCCCGGGCCTATTTTCTAATATCCACAAAATGAAGGCAA | |
| | TAATTGTACTACTCATGGTAGTAACATCCAATGCAGATCGAATCTGCA | |
| | CTGGGATAACATCGTCAAACTCACCACATGTCGTCAAAACTGCTACT | |
| | CAAGGGGAGGTCAACGTGACCGGTGTAATACCACTGACAACAACAC | |
| | CCACCAAATCTCATTTTGCAAATCTCAAAGGAACAGAAACCAGGGGG | |
| | AAACTATGCCCAAAATGCCTCAACTGCACAGATCTGGATGTAGCCTT | |
| | GGGCAGACCAAAATGCACAGGGAAAATACCCTCTGCAAGGGTTTCAA | |
| | TACTCCATGAAGTCAGACCTGTTACATCTGGGTGCTTTCCTATAATGC | |
| | ACGATAGAACAAAAATTAGACAGCTGCCTAACCTTCTCCGAGGATAC | |
| | GAACATGTCAGGTTATCAACTCACAACGTTATCAATGCAAAAGATGCA | |
| | CCAGGAAGACCCTACGAAATTGGAACCTCAGGGTCTTGCCCTAACAT | |
| | TACCAATGGAAACGGATTCTTCGCAACAATGGCTTGGGCCGTCCCAA | |
| | AAAACAAAACAGCAACAAATCCATTAACAATAGAAGTACCATACATTT | |
| | GTACAGAAGGAGAAGACCAAATTACCGTTTGGGGGTTCCACTCTGAC | |
| | AACGAGACCCAAATGGCAAAGCTCTATGGGGACTCAAAGCCCCAGA | |
| | AGTTCACCTCATCTGCCAACGGAGTGACCACACATTACGTTTCACAG | |
| | ATTGGTGGCTTCCCAAATCAAACAGAAGACGGAGGACTACCACAAAG | |
| | TGGCAGAATTGTTGTTGATTACATGGTGCAGAAATCTGGAAAAACAG | |
| | GAACAATTACCTATCAAAGAGGTATTTTATTGCCTCAAAAAGTGTGGT | |
| | GCGCAAGTGGCAGGAGCAAGGTAATAAAAGGATCCTTGCCCTTAATT | |
| | GGAGAAGCAGATTGCCTCCATGAAAAATACGGTGGATTAAACAAAAG | |
| | CAAGCCTTACTACACAGGGGAACATGCAAAGGCCATAGGAAATTGCC | |
| | CAATATGGGTGAAAACACCCTTGAAGCTGGCCAATGGAACCAAATAT | |
| | AGACCCCCTGCAAAACTATTAAAGGAAAGAGGTTTCTTCGGAGCCAT | |
| | TGCTGGTTTCTTAGAGGGAGGATGGGAAGGAATGATTGCAGGTTGG | |
| | CACGGATACACATCCCATGGGGCACATGGAGTAGCGGTGGCAGCTG | |
| | ACCTTAAGAGCACTCAAGAGGCCATAAACAAGATAACAAAAAATCTCA | |
| | ACTCTTTGAGTGAGCTGGAAGTAAAGAATCTTCAAAGACTAAGCGGT | |
| | GCCATGGATGAACTCCACAACGAAATACTAGAACTAGATGAGAAAGT | |
| | GGATGATCTCAGAGCTGACACAATAAGCTCACAAATAGAACTCGCAG | |
| | TCCTGCTTTCCAATGAAGGAATAATAAACAGTGAAGATGAACATCTCT | |
| | TGGCGCTTGAAAGAAAGCTGAAGAAAATGCTGGGCCCCTCTGCTGTA | |
| | GAGATAGGGAATGGATGCTTTGAAACCAAACACAAGTGCAACCAGAC | |
| | CTGCCTCGACAGAATAGCTGCTGGTACCTTTGATGCAGGAGAATTTT | |
| | CTCTCCCCACCTTTGATTCACTGAATATTACTGCTGCATCTTTAAATG | |
| | ACGACGGATTGGACAATCATACTATACTGCTTTACTACTCAACTGCTG | |
| | CCTCCAGTTTGGCTGTAACACTGATGATAGCTATCTTTGTTGTTTATA | |
| | TGGTCTCCAGAGACAATGTTTCTTGCTCCATTTGTCTATAAGGAAAGT | |
| | TAAGCCCTGTATTTTCCTTTATTGTAGTGCTTGTTTGCTTGTTGTCATT | |
| | ACAAAGAAACGTTATTGAAAAATGGAAGCGGAGCCACGAACTTCTCT | |
| | CTGTTAAAGCAAGCAGGAGATGTTGAAGAAAACCCCGGGCCTTTCGG | |
| | AGCTATTGCTGGTTTCTTGGAAGGAGGATGGGAAGGAATGATTGCAG | |
| | GTTGGCACGGATACACATCTCATGGAGCACATGGAGTAGCAGTGGC | |
| | AGCAGACCTTAAGAGTACCCATGATGACTCGAGCTGGTACTGCATGC | |
| | ACGCAATGCTAGCTGCCCCTTTCCCGTCCTGGGTACCCCGAGTCTCC | |
| | CCCGACCTCGGGTCCCAGGTATGCTCCCACCTCCACCTGCCCCACT | |
| | CACCACCTCTGCTAGTTCCAGACACCTCCCAAGCACGCAGCAATGCA | |
| | GCTCAAAACGCTTAGCCTAGCCACACCCCCACGGGAAACAGCAGTG | |
| | ATTAACCTTTAGCAATAAACGAAAGTTTAACTAAGCTATACTAACCCC | |
| | AGGGTTGGTCAATTTCGTGCCAGCCACACCCTGGAGCTAGCAAAAAA | |
| | AA | |
| Pan-CoV-Flu | CTCGACATTGATTATTGACTAGTTATTAATAGTAATCAATTACGGGGT | 402 |
| Vaccine #14 | CATTAGTTCATAGCCCATATATGGAGTTCCGCGTTACATAACTTACGG | |
| CoV-Wuhan | TAAATGGCCCGCCTGGCTGACCGCCCAACGACCCCCGCCCATTGAC | |
| Spike(2 proline | GTCAATAATGACGTATGTTCCCATAGTAACGCCAATAGGGACTTTCCA | |
| substitution)- | TTGACGTCAATGGGTGGAGTATTTACGGTAAACTGCCCACTTGGCAG | |
| influenza (HA) | TACATCAAGTGTATCATATGCCAAGTACGCCCCCTATTGACGTCAATG | |
| | ACGGTAAATGGCCCGCCTGGCATTATGCCCAGTACATGACCTTATGG | |
| | GACTTTCCTACTTGGCAGTACATCTACGTATTAGTCATCGCTATTACC | |
| | ATGGTCGAGGTGAGCCCCACGTTCTGCTTCACTCTCCCCATCTCCCC | |
| | CCCCTCCCCACCCCCAATTTTGTATTTATTTATTTTTTAATTATTTTGT | |
| | GCAGCGATGGGGGCGGGGGGGGGGGGGGGGCGCGCGCCAGGCG | |
| | GGGCGGGGCGGGGCGAGGGGCGGGGCGGGGCGAGGCGGAGAGG | |
| | TGCGGCGGCAGCCAATCAGAGCGGCGCGCTCCGAAAGTTTCCTTTT | |
| | ATGGCGAGGCGGCGGCGGCGGCGGCCCTATAAAAAGCGAAGCGCG | |
| | CGGCGGGCGGGAGTCGCTGCGCGCTGCCTTCGCCCCGTGCCCCGC | |
| | TCCGCCGCCGCCTCGCGCCGCCCGCCCCGGCTCTGACTGACCGCG | |
| | TTACTCCCACAGGTGAGCGGGCGGGACGGCCCTTCTCCTCCGGGCT | |
| | GTAATTAGCGCTTGGTTTAATGACGGCTTGTTTCTTTTCTGTGGCTGC | |
| | GTGAAAGCCTTGAGGGGCTCCGGGAGGGCCCTTTGTGCGGGGGGA | |
| | GCGGCTCGGGGGGTGCGTGCGTGTGTGTGTGCGTGGGGAGCGCCG | |
| | CGTGCGGCTCCGCGCTGCCCGGCGGCTGTGAGCGCTGCGGGCGCG | |
| | GCGCGGGGCTTTGTGCGCTCCGCAGTGTGCGCGAGGGGAGCGCGG | |
| | CCGGGGGCGGTGCCCCGCGGTGCGGGGGGGGCTGCGAGGGGAAC | |
| | AAAGGCTGCGTGCGGGGTGTGTGCGTGGGGGGGTGAGCAGGGGGT | |
| | GTGGGCGCGTCGGTCGGGCTGCAACCCCCCCTGCACCCCCCTCCC | |
| | CGAGTTGCTGAGCACGGCCCGGCTTCGGGTGCGGGGCTCCGTACG | |
| | GGGCGTGGCGCGGGGCTCGCCGTGCCGGGCGGGGGGTGGCGGCA | |
| | GGTGGGGGTGCCGGGCGGGGCGGGGCCGCCTCGGGCCGGGGAG | |
| | GGCTCGGGGGAGGGGCGCGGCGGCCCCCGGAGCGCCGGCGGCTG | |
| | TCGAGGCGCGGCGAGCCGCAGCCATTGCCTTTTATGGTAATCGTGC | |
| | GAGAGGGCGCAGGGACTTCCTTTGTCCCAAATCTGTGCGGAGCCGA | |
| | AATCTGGGAGGCGCCGCCGCACCCCCTCTAGCGGGCGCGGGGCGA | |
| | AGCGGTGCGGCGCCGGCAGGAAGGAAATGGGCGGGGAGGGCCTTC | |
| | GTGCGTCGCCGCGCCGCCGTCCCCTTCTCCCTCTCCAGCCTCGGGG | |
| | CTGTCCGCGGGGGGACGGCTGCCTTCGGGGGGGACGGGGCAGGG | |
| | CGGGGTTCGGCTTCTGGCGTGTGACCGGCGGCTCTAGAGCCTCTGC | |
| | TAACCATGTTCATGCCTTCTTCTTTTTCCTACAGCTCCTGGGCAACGT | |
| | GCTGGTTATTGTGCTGTCTCATCATTTTGGCAAAGAATTGGAGAATAA | |
| | ACTAGTATTCTTCTGGTCCCCACAGACTCAGAGAGAACCCGCCACCA | |
| | TGTTCGTGTTCCTGGTGCTGCTGCCCCTGGTGAGCAGCCAGTGCGT | |
| | GAACCTGACCACCAGGACCCAGCTGCCCCCCGCCTACACCAACAGC | |
| | TTCACCAGGGGCGTGTACTACCCCGACAAGGTGTTCAGGAGCAGCG | |
| | TGCTGCACAGCACCCAGGACCTGTTCCTGCCCTTCTTCAGCAACGTG | |
| | ACCTGGTTCCACGCCATCCACGTGAGCGGCACCAACGGCACCAAGA | |
| | GGTTCGACAACCCCGTGCTGCCCTTCAACGACGGCGTGTACTTCGC | |
| | CAGCACCGAGAAGAGCAACATCATCAGGGGCTGGATCTTCGGCACC | |
| | ACCCTGGACAGCAAGACCCAGAGCCTGCTGATCGTGAACAACGCCA | |
| | CCAACGTGGTGATCAAGGTGTGCGAGTTCCAGTTCTGCAACGACCC | |
| | CTTCCTGGGCGTGTACTACCACAAGAACAACAAGAGCTGGATGGAGA | |
| | GCGAGTTCAGGGTGTACAGCAGCGCCAACAACTGCACCTTCGAGTA | |
| | CGTGAGCCAGCCCTTCCTGATGGACCTGGAGGGCAAGCAGGGCAAC | |
| | TTCAAGAACCTGAGGGAGTTCGTGTTCAAGAACATCGACGGCTACTT | |
| | CAAGATCTACAGCAAGCACACCCCCATCAACCTGGTGAGGGACCTG | |
| | CCCCAGGGCTTCAGCGCCCTGGAGCCCCTGGTGGACCTGCCCATCG | |
| | GCATCAACATCACCAGGTTCCAGACCCTGCTGGCCCTGCACAGGAG | |
| | CTACCTGACCCCCGGCGACAGCAGCAGCGGCTGGACCGCCGGCGC | |
| | CGCCGCCTACTACGTGGGCTACCTGCAGCCCAGGACCTTCCTGCTG | |
| | AAGTACAACGAGAACGGCACCATCACCGACGCCGTGGACTGCGCCC | |
| | TGGACCCCCTGAGCGAGACCAAGTGCACCCTGAAGAGCTTCACCGT | |
| | GGAGAAGGGCATCTACCAGACCAGCAACTTCAGGGTGCAGCCCACC | |
| | GAGAGCATCGTGAGGTTCCCCAACATCACCAACCTGTGCCCCTTCG | |
| | GCGAGGTGTTCAACGCCACCAGGTTCGCCAGCGTGTACGCCTGGAA | |
| | CAGGAAGAGGATCAGCAACTGCGTGGCCGACTACAGCGTGCTGTAC | |
| | AACAGCGCCAGCTTCAGCACCTTCAAGTGCTACGGCGTGAGCCCCA | |
| | CCAAGCTGAACGACCTGTGCTTCACCAACGTGTACGCCGACAGCTTC | |
| | GTGATCAGGGGCGACGAGGTGAGGCAGATCGCCCCCGGCCAGACC | |
| | GGCAAGATCGCCGACTACAACTACAAGCTGCCCGACGACTTCACCG | |
| | GCTGCGTGATCGCCTGGAACAGCAACAACCTGGACAGCAAGGTGGG | |
| | CGGCAACTACAACTACCTGTACAGGCTGTTCAGGAAGAGCAACCTGA | |
| | AGCCCTTCGAGAGGGACATCAGCACCGAGATCTACCAGGCCGGCAG | |
| | CACCCCCTGCAACGGCGTGGAGGGCTTCAACTGCTACTTCCCCCTG | |
| | CAGAGCTACGGCTTCCAGCCCACCAACGGCGTGGGCTACCAGCCCT | |
| | ACAGGGTGGTGGTGCTGAGCTTCGAGCTGCTGCACGCCCCCGCCAC | |
| | CGTGTGCGGCCCCAAGAAGAGCACCAACCTGGTGAAGAACAAGTGC | |
| | GTGAACTTCAACTTCAACGGCCTGACCGGCACCGGCGTGCTGACCG | |
| | AGAGCAACAAGAAGTTCCTGCCCTTCCAGCAGTTCGGCAGGGACAT | |
| | CGCCGACACCACCGACGCCGTGAGGGACCCCCAGACCCTGGAGAT | |
| | CCTGGACATCACCCCCTGCAGCTTCGGCGGCGTGAGCGTGATCACC | |
| | CCCGGCACCAACACCAGCAACCAGGTGGCCGTGCTGTACCAGGACG | |
| | TGAACTGCACCGAGGTGCCCGTGGCCATCCACGCCGACCAGCTGAC | |
| | CCCCACCTGGAGGGTGTACAGCACCGGCAGCAACGTGTTCCAGACC | |
| | AGGGCCGGCTGCCTGATCGGCGCCGAGCACGTGAACAACAGCTAC | |
| | GAGTGCGACATCCCCATCGGCGCCGGCATCTGCGCCAGCTACCAGA | |
| | CCCAGACCAACAGCCCCAGGAGGGCCAGGAGCGTGGCCAGCCAGA | |
| | GCATCATCGCCTACACCATGAGCCTGGGCGCCGAGAACAGCGTGGC | |
| | CTACAGCAACAACAGCATCGCCATCCCCACCAACTTCACCATCAGCG | |
| | TGACCACCGAGATCCTGCCCGTGAGCATGACCAAGACCAGCGTGGA | |
| | CTGCACCATGTACATCTGCGGCGACAGCACCGAGTGCAGCAACCTG | |
| | CTGCTGCAGTACGGCAGCTTCTGCACCCAGCTGAACAGGGCCCTGA | |
| | CCGGCATCGCCGTGGAGCAGGACAAGAACACCCAGGAGGTGTTCGC | |
| | CCAGGTGAAGCAGATCTACAAGACCCCCCCCATCAAGGACTTCGGC | |
| | GGCTTCAACTTCAGCCAGATCCTGCCCGACCCCAGCAAGCCCAGCA | |
| | AGAGGAGCTTCATCGAGGACCTGCTGTTCAACAAGGTGACCCTGGC | |
| | CGACGCCGGCTTCATCAAGCAGTACGGCGACTGCCTGGGCGACATC | |
| | GCCGCCAGGGACCTGATCTGCGCCCAGAAGTTCAACGGCCTGACCG | |
| | TGCTGCCCCCCCTGCTGACCGACGAGATGATCGCCCAGTACACCAG | |
| | CGCCCTGCTGGCCGGCACCATCACCAGCGGCTGGACCTTCGGCGC | |
| | CGGCGCCGCCCTGCAGATCCCCTTCGCCATGCAGATGGCCTACAGG | |
| | TTCAACGGCATCGGCGTGACCCAGAACGTGCTGTACGAGAACCAGA | |
| | AGCTGATCGCCAACCAGTTCAACAGCGCCATCGGCAAGATCCAGGA | |
| | CAGCCTGAGCAGCACCGCCAGCGCCCTGGGCAAGCTGCAGGACGT | |
| | GGTGAACCAGAACGCCCAGGCCCTGAACACCCTGGTGAAGCAGCTG | |
| | AGCAGCAACTTCGGCGCCATCAGCAGCGTGCTGAACGACATCCTGA | |
| | GCAGGCTGGACCCCCCCGAGGCCGAGGTGCAGATCGACAGGCTGA | |
| | TCACCGGCAGGCTGCAGAGCCTGCAGACCTACGTGACCCAGCAGCT | |
| | GATCAGGGCCGCCGAGATCAGGGCCAGCGCCAACCTGGCCGCCAC | |
| | CAAGATGAGCGAGTGCGTGCTGGGCCAGAGCAAGAGGGTGGACTTC | |
| | TGCGGCAAGGGCTACCACCTGATGAGCTTCCCCCAGAGCGCCCCCC | |
| | ACGGCGTGGTGTTCCTGCACGTGACCTACGTGCCCGCCCAGGAGAA | |
| | GAACTTCACCACCGCCCCCGCCATCTGCCACGACGGCAAGGCCCAC | |
| | TTCCCCAGGGAGGGCGTGTTCGTGAGCAACGGCACCCACTGGTTCG | |
| | TGACCCAGAGGAACTTCTACGAGCCCCAGATCATCACCACCGACAAC | |
| | ACCTTCGTGAGCGGCAACTGCGACGTGGTGATCGGCATCGTGAACA | |
| | ACACCGTGTACGACCCCCTGCAGCCCGAGCTGGACAGCTTCAAGGA | |
| | GGAGCTGGACAAGTACTTCAAGAACCACACCAGCCCCGACGTGGAC | |
| | CTGGGCGACATCAGCGGCATCAACGCCAGCGTGGTGAACATCCAGA | |
| | AGGAGATCGACAGGCTGAACGAGGTGGCCAAGAACCTGAACGAGAG | |
| | CCTGATCGACCTGCAGGAGCTGGGCAAGTACGAGCAGTACATCAAG | |
| | TGGCCCTGGTACATCTGGCTGGGCTTCATCGCCGGCCTGATCGCCA | |
| | TCGTGATGGTGACCATCATGCTGTGCTGCATGACCAGCTGCTGCAGC | |
| | TGCCTGAAGGGCTGCTGCAGCTGCGGCAGCTGCTGCAAGTTCGACG | |
| | AGGACGACAGCGAGCCCGTGCTGAAGGGCGTGAAGCTGCACTACAC | |
| | CGGAAGCGGAGCCACGAACTTCTCTCTGTTAAAGCAAGCAGGAGAT | |
| | GTTGAAGAAAACCCCGGGCCTATGAAGGCAATACTAGTAGTTCTGCT | |
| | ATATACATTTGCAACCGCAAATGCAGACACATTATGTATAGGTTATCA | |
| | TGCGAACAACTCAACAGACACTGTAGACACAGTACTAGAAAAGAATG | |
| | TAACAGTAACACACTCTGTTAACCTTCTAGAAGACAAGCATAACGGGA | |
| | AACTATGCAAACTAAGAGGGGTAGCCCCATTGCATTTGGGTAAATGT | |
| | AACATTGCTGGCTGGATCCTGGGAAATCCAGAGTGTGAATCACTCTC | |
| | CACAGCAAGCTCATGGTCCTACATTGTGGAAACATCTAGTTCAGACA | |
| | ATGGAACGTGTTACCCAGGAGATTTCATCGATTATGAGGAGCTAAGA | |
| | GAGCAATTGAGCTCAGTGTCATCATTTGAAAGGTTTGAGATATTCCCC | |
| | AAGACAAGTTCATGGCCCAATCATGACTCGAACAAAGGTGTAACGAC | |
| | AGCATGTCCTCATGCTGGAGCAAAAAGCTTCTACAAAAATTTAATATG | |
| | GCTAGTTAAAAAAGGAAATTCATACCCAAAGCTCAGCAAATCCTACAT | |
| | TAATGATAAAGGGAAAGAAGTCCTCGTGCTATGGGGCATTCACCATC | |
| | CACCTACTAGTGCTGACCAACAAAGTCTCTATCAGAATGCAGATGCA | |
| | TATGTTTTTGTGGGGACATCAAGATACAGCAAGAAGTTCAAGCCGGA | |
| | AATAGCAATAAGGCCCAAAGTGAGGGATCAAGAAGGGAGAATGAACT | |
| | ATTACTGGACACTAGTAGAGCCGGGAGACAAAATAACATTCGAAGCA | |
| | ACTGGAAATCTAGTGGTACCGAGATATGCATTCGCAATGGAAAGAAA | |
| | TGCTGGATCTGGTATTATCATTTCAGATACACCAGTCCACGATTGCAA | |
| | TACAACTTGTCAGACACCCAAGGGTGCTATAAACACCAGCCTCCCAT | |
| | TTCAGAACATACATCCGATCACAATTGGAAAATGTCCAAAATATGTAA | |
| | AAAGCACAAAATTGAGACTGGCCACAGGATTGAGGAATGTCCCGTCC | |
| | ATTCAATCTAGAGGCCTATTTGGGGCCATTGCCGGTTTCATTGAAGG | |
| | GGGGTGGACAGGGATGGTAGATGGATGGTACGGTTATCACCATCAA | |
| | AATGAGCAGGGGTCAGGATATGCAGCCGACCTGAAGAGCACACAGA | |
| | ATGCCATTGACGAGATTACTAACAAAGTAAACTCTGTTATTGAAAAAA | |
| | TGAATACACAGTTCACAGCAGTAGGTAAAGAGTTCAACCACCTGGAA | |
| | AAAAGAATAGAGAATTTAAATAAAAAAGTTGATGATGGTTTCCTGGAC | |
| | ATTTGGACTTACAATGCCGAACTGTTGGTTCTATTGGAAAATGAAAGA | |
| | ACTTTGGACTACCACGATTCAAAGGTGAAGAACTTATATGAAAAGGTA | |
| | AGAAGCCAGTTAAAAAACAATGCCAAGGAAATTGGAAACGGCTGCTT | |
| | TGAATTTTACCACAAATGTGATAACACGTGCATGGAAAGTGTCAAAAA | |
| | TGGGACTTATGACTACCCAAAATACTCAGAGGAAGCAAAATTAAACA | |
| | GAGAAGAAATAGATGGGGTAAAGCTGGAATCAACAAGGATTTACCAG | |
| | ATTTTGGCGATCTATTCAACCGTCGCCAGTTCATTGGTACTGGTAGTC | |
| | TCCCTGGGGGCAATCAGTTTCTGGATGTGCTCTAATGGGTCTCTACA | |
| | GTGTAGAATATGTATTTAAGGAAGCGGAGCCACGAACTTCTCTCTGTT | |
| | AAAGCAAGCAGGAGATGTTGAAGAAAACCCCGGGCCTAGCAAAAGC | |
| | AGGGGATAATTCTATTAACCATGAAGACTATCATTGCTTTGAGCTACA | |
| | TTCTATGTCTGGTTTTCGCTCAAAAACTTCCTGGAAATGACAATAGCA | |
| | CTGCAACGCTGTGCCTTGGGCACCATGCAGTACCAAACGGAACGAT | |
| | AGTGAAAACAATCACGAATGACCAAATTGAAGTTACTAATGCTACTGA | |
| | GCTGGTTCAGAATTCCTCAATAGGTGAAATATGCGACAGTCCTCATC | |
| | AGATCCTTGATGGAGAAAACTGCACACTAATAGATGCTCTATTGGGA | |
| | GACCCTCAGTGTGATGGCTTTCAAAATAAGAAATGGGACCTTTTTGTT | |
| | GAACGAAGCAAAGCCTACAGCAACTGTTACCCTTATGATGTGCCGGA | |
| | TTATGCCTCCCTTAGGTCACTAGTTGCCTCATCCGGCACACTGGAGT | |
| | TTAACAATGAAAGCTTCAATTGGGCTGGAGTCACTCAAAACGGAACA | |
| | AGTTCTGCTTGCATAAGGGGATCTAATAGTAGTTTCTTTAGTAGATTA | |
| | AATTGGTTGACCCACTTAAACTTCAAGTACCCAGCATTGAACGTGACT | |
| | ATGCCAAACAATGAACAATTTGACAAATTGTACATTTGGGGGGTTCAC | |
| | CACCCGGGTACGGACAAGGACCAAATCTTCCTGTATGCTCAATCATC | |
| | AGGAAGAATCACAGTATCTACCAAAAGAAGCCAACAAGCTGTAATCC | |
| | CGAATATCGGATCTAGACCCAGAATAAGGAATATCCCTAGCAGAATA | |
| | AGCATCTATTGGACAATAGTAAAACCGGGAGACATACTTTTGATTAAC | |
| | AGCACAGGGAATCTAATTGCTCCTAGGGGTTACTTCAAAATACGAAG | |
| | TGGGAAAAGCTCAATAATGAGATCAGATGCACCCATTGGCAAATGCA | |
| | AGTCTGAATGCATCACTCCAAATGGAAGCATTCCCAATGACAAACCAT | |
| | TCCAAAATGTAAACAGGATCACATACGGGGCCTGTCCCAGATATGTT | |
| | AAGCAAAGCACTCTGAAATTGGCAACAGGAATGCGAAATGTACCAGA | |
| | GAAACAAACTAGAGGCATATTTGGCGCAATAGCGGGTTTCATAGAAA | |
| | ATGGTTGGGAGGGAATGGTGGATGGTTGGTACGGTTTCAGGCATCA | |
| | AAATTCTGAGGGAAGAGGACAAGCAGCAGATCTCAAAAGCACTCAAG | |
| | CAGCAATCGATCAAATCAATGGGAAGCTGAATCGATTGATCGGGAAA | |
| | ACCAACGAGAAATTCCATCAGATTGAGAAAGAATTCTCAGAAGTAGAA | |
| | GGGAGAATTCAGGACCTTGAGAAATATGTTGAGGACACAAAAATAGA | |
| | TCTCTGGTCATACAACGCAGAGCTTCTTGTTGCCCTGGAAAACCAAC | |
| | ATACAATTGATCTAACTGACTCAGAAATGAACAAACTGTTTGAAAAAA | |
| | CAAAGAAGCAACTGAGGGAAAATGCTGAGGATATGGGCAATGGTTGT | |
| | TTCAAAATATACCACAAATGTGACAATGCCTGCATAGGATCAATCAGA | |
| | AATGGAACTTATGACCACGATGTATACAGGGATGAAGCATTAAACAA | |
| | CCGGTTCCAGATCAAGGGAGTTGAGCTGAAGTCAGGGTACAAAGATT | |
| | GGATCCTATGGATTTCCTTTGCCATATCATGTTTTTTGCTTTGTGTTGC | |
| | TTTGTTGGGGTTCATCATGTGGGCCTGCCAAAAGGGCAACATTAGGT | |
| | GCAACATTTGCATTTGAGTGCATTAATTAAAAACACCCTTGTTTCTACT | |
| | GGAAGCGGAGCCACGAACTTCTCTCTGTTAAAGCAAGCAGGAGATGT | |
| | TGAAGAAAACCCCGGGCCTATTTTGTAATATCCACAAAATGAAGGCAA | |
| | TAATTGTACTACTCATGGTAGTAACATCCAATGCAGATCGAATCTGCA | |
| | CTGGGATAACATCGTCAAACTCACCACATGTCGTCAAAACTGCTACT | |
| | CAAGGGGAGGTCAACGTGACCGGTGTAATACCACTGACAACAACAC | |
| | CCACCAAATCTCATTTTGCAAATCTCAAAGGAACAGAAACCAGGGGG | |
| | AAACTATGCCCAAAATGCCTCAACTGCACAGATCTGGATGTAGCCTT | |
| | GGGCAGACCAAAATGCACAGGGAAAATACCCTCTGCAAGGGTTTCAA | |
| | TACTCCATGAAGTCAGACCTGTTACATCTGGGTGCTTTCCTATAATGC | |
| | ACGATAGAACAAAAATTAGACAGCTGCCTAACCTTCTCCGAGGATAC | |
| | GAACATGTCAGGTTATCAACTCACAACGTTATCAATGCAAAAGATGCA | |
| | CCAGGAAGACCCTACGAAATTGGAACCTCAGGGTCTTGCCCTAACAT | |
| | TACCAATGGAAACGGATTCTTCGCAACAATGGCTTGGGCCGTCCCAA | |
| | AAAACAAAACAGCAACAAATCCATTAACAATAGAAGTACCATACATTT | |
| | GTACAGAAGGAGAAGACCAAATTACCGTTTGGGGGTTCCACTCTGAC | |
| | AACGAGACCCAAATGGCAAAGCTCTATGGGGACTCAAAGCCCCAGA | |
| | AGTTCACCTCATCTGCCAACGGAGTGACCACACATTACGTTTCACAG | |
| | ATTGGTGGCTTCCCAAATCAAACAGAAGACGGAGGACTACCACAAAG | |
| | TGGCAGAATTGTTGTTGATTACATGGTGCAGAAATCTGGAAAAACAG | |
| | GAACAATTACCTATCAAAGAGGTATTTTATTGCCTCAAAAAGTGTGGT | |
| | GCGCAAGTGGCAGGAGCAAGGTAATAAAAGGATCCTTGCCCTTAATT | |
| | GGAGAAGCAGATTGCCTCCATGAAAAATACGGTGGATTAAACAAAAG | |
| | CAAGCCTTACTACACAGGGGAACATGCAAAGGCCATAGGAAATTGCC | |
| | CAATATGGGTGAAAACACCCTTGAAGCTGGCCAATGGAACCAAATAT | |
| | AGACCCCCTGCAAAACTATTAAAGGAAAGAGGTTTCTTCGGAGCCAT | |
| | TGCTGGTTTCTTAGAGGGAGGATGGGAAGGAATGATTGCAGGTTGG | |
| | CACGGATACACATCCCATGGGGCACATGGAGTAGCGGTGGCAGCTG | |
| | ACCTTAAGAGCACTCAAGAGGCCATAAACAAGATAACAAAAAATCTCA | |
| | ACTCTTTGAGTGAGCTGGAAGTAAAGAATCTTCAAAGACTAAGCGGT | |
| | GCCATGGATGAACTCCACAACGAAATACTAGAACTAGATGAGAAAGT | |
| | GGATGATCTCAGAGCTGACACAATAAGCTCACAAATAGAACTCGCAG | |
| | TCCTGCTTTCCAATGAAGGAATAATAAACAGTGAAGATGAACATCTCT | |
| | TGGCGCTTGAAAGAAAGCTGAAGAAAATGCTGGGCCCCTCTGCTGTA | |
| | GAGATAGGGAATGGATGCTTTGAAACCAAACACAAGTGCAACCAGAC | |
| | CTGCCTCGACAGAATAGCTGCTGGTACCTTTGATGCAGGAGAATTTT | |
| | CTCTCCCCACCTTTGATTCACTGAATATTACTGCTGCATCTTTAAATG | |
| | ACGACGGATTGGACAATCATACTATACTGCTTTACTACTCAACTGCTG | |
| | CCTCCAGTTTGGCTGTAACACTGATGATAGCTATCTTTGTTGTTTATA | |
| | TGGTCTCCAGAGACAATGTTTCTTGCTCCATTTGTCTATAAGGAAAGT | |
| | TAAGCCCTGTATTTTCCTTTATTGTAGTGCTTGTTTGCTTGTTGTCATT | |
| | ACAAAGAAACGTTATTGAAAAATGGAAGCGGAGCCACGAACTTCTCT | |
| | CTGTTAAAGCAAGCAGGAGATGTTGAAGAAAACCCCGGGCCTTTCGG | |
| | AGCTATTGCTGGTTTCTTGGAAGGAGGATGGGAAGGAATGATTGCAG | |
| | GTTGGCACGGATACACATCTCATGGAGCACATGGAGTAGCAGTGGC | |
| | AGCAGACCTTAAGAGTACCCATGATGACTCGAGCTGGTACTGCATGC | |
| | ACGCAATGCTAGCTGCCCCTTTCCCGTCCTGGGTACCCCGAGTCTCC | |
| | CCCGACCTCGGGTCCCAGGTATGCTCCCACCTCCACCTGCCCCACT | |
| | CACCACCTCTGCTAGTTCCAGACACCTCCCAAGCACGCAGCAATGCA | |
| | GCTCAAAACGCTTAGCCTAGCCACACCCCCACGGGAAACAGCAGTG | |
| | ATTAACCTTTAGCAATAAACGAAAGTTTAACTAAGCTATACTAACCCC | |
| | AGGGTTGGTCAATTTCGTGCCAGCCACACCCTGGAGCTAGCAAAAAA | |
| | AA | |
| Pan-CoV-Flu | TAGTTATTAATAGTAATCAATTACGGGGTCATTAGTTCATAGCCCATAT | 403 |
| Vaccine #15 | ATGGAGTTCCGCGTTACATAACTTACGGTAAATGGCCCGCCTGGCTG | |
| CoV- Omicron | ACCGCCCAACGACCCCCGCCCATTGACGTCAATAATGACGTATGTTC | |
| BA.2 | CCATAGTAACGCCAATAGGGACTTTCCATTGACGTCAATGGGTGGAG | |
| Spike(HexaPro)- | TATTTACGGTAAACTGCCCACTTGGCAGTACATCAAGTGTATCATATG | |
| Influenza (HA) | CCAAGTACGCCCCCTATTGACGTCAATGACGGTAAATGGCCCGCCTG | |
| | GCATTATGCCCAGTACATGACCTTATGGGACTTTCCTACTTGGCAGTA | |
| | CATCTACGTATTAGTCATCGCTATTACCATGGTGATGCGGTTTTGGCA | |
| | GTACATCAATGGGCGTGGATAGCGGTTTGACTCACGGGGATTTCCAA | |
| | GTCTCCACCCCATTGACGTCAATGGGAGTTTGTTTTGGCACCAAAAT | |
| | CAACGGGACTTTCCAAAATGTCGTAACAACTCCGCCCCATTGACGCA | |
| | AATGGGCGGTAGGCGTGTACGGTGGGAGGTCTATATAAGCAGAGCT | |
| | GGTTTAGTGAACCGTCAGATCGAGAATAAACTAGTATTCTTCTGGTCC | |
| | CCACAGACTCAGAGAGAACCCGCCACCATGTTCGTGTTCCTGGTGCT | |
| | GCTGCCCCTGGTGAGCAGCCAGTGCGTGAACCTGATCACCAGGACC | |
| | CAGAGCTACACCAACAGCTTCACCAGGGGCGTGTACTACCCCGACA | |
| | AGGTGTTCAGGAGCAGCGTGCTGCACAGCACCCAGGACCTGTTCCT | |
| | GCCCTTCTTCAGCAACGTGACCTGGTTCCACGCCATCCACGTGAGC | |
| | GGCACCAACGGCACCAAGAGGTTCGACAACCCCGTGCTGCCCTTCA | |
| | ACGACGGCGTGTACTTCGCCAGCACCGAGAAGAGCAACATCATCAG | |
| | GGGCTGGATCTTCGGCACCACCCTGGACAGCAAGACCCAGAGCCTG | |
| | CTGATCGTGAACAACGCCACCAACGTGGTGATCAAGGTGTGCGAGTT | |
| | CCAGTTCTGCAACGACCCCTTCCTGGACGTGTACTACCACAAGAACA | |
| | ACAAGAGCTGGATGGAGAGCGAGTTCAGGGTGTACAGCAGCGCCAA | |
| | CAACTGCACCTTCGAGTACGTGAGCCAGCCCTTCCTGATGGACCTG | |
| | GAGGGCAAGCAGGGCAACTTCAAGAACCTGAGGGAGTTCGTGTTCA | |
| | AGAACATCGACGGCTACTTCAAGATCTACAGCAAGCACACCCCCATC | |
| | AACCTGGGCAGGGACCTGCCCCAGGGCTTCAGCGCCCTGGAGCCC | |
| | CTGGTGGACCTGCCCATCGGCATCAACATCACCAGGTTCCAGACCCT | |
| | GCTGGCCCTGCACAGGAGCTACCTGACCCCCGGCGACAGCAGCAG | |
| | CGGCTGGACCGCCGGCGCCGCCGCCTACTACGTGGGCTACCTGCA | |
| | GCCCAGGACCTTCCTGCTGAAGTACAACGAGAACGGCACCATCACC | |
| | GACGCCGTGGACTGCGCCCTGGACCCCCTGAGCGAGACCAAGTGC | |
| | ACCCTGAAGAGCTTCACCGTGGAGAAGGGCATCTACCAGACCAGCA | |
| | ACTTCAGGGTGCAGCCCACCGAGAGCATCGTGAGGTTCCCCAACAT | |
| | CACCAACCTGTGCCCCTTCGACGAGGTGTTCAACGCCACCAGGTTC | |
| | GCCAGCGTGTACGCCTGGAACAGGAAGAGGATCAGCAACTGCGTGG | |
| | CCGACTACAGCGTGCTGTACAACTTCGCCCCCTTCTTCGCCTTCAAG | |
| | TGCTACGGCGTGAGCCCCACCAAGCTGAACGACCTGTGCTTCACCA | |
| | ACGTGTACGCCGACAGCTTCGTGATCAGGGGCAACGAGGTGAGCCA | |
| | GATCGCCCCCGGCCAGACCGGCAACATCGCCGACTACAACTACAAG | |
| | CTGCCCGACGACTTCACCGGCTGCGTGATCGCCTGGAACAGCAACA | |
| | AGCTGGACAGCAAGGTGGGCGGCAACTACAACTACCTGTACAGGCT | |
| | GTTCAGGAAGAGCAACCTGAAGCCCTTCGAGAGGGACATCAGCACC | |
| | GAGATCTACCAGGCCGGCAACAAGCCCTGCAACGGCGTGGCCGGCT | |
| | TCAACTGCTACTTCCCCCTGAGGAGCTACGGCTTCAGGCCCACCTAC | |
| | GGCGTGGGCCACCAGCCCTACAGGGTGGTGGTGCTGAGCTTCGAG | |
| | CTGCTGCACGCCCCCGCCACCGTGTGCGGCCCCAAGAAGAGCACCA | |
| | ACCTGGTGAAGAACAAGTGCGTGAACTTCAACTTCAACGGCCTGACC | |
| | GGCACCGGCGTGCTGACCGAGAGCAACAAGAAGTTCCTGCCCTTCC | |
| | AGCAGTTCGGCAGGGACATCGCCGACACCACCGACGCCGTGAGGG | |
| | ACCCCCAGACCCTGGAGATCCTGGACATCACCCCCTGCAGCTTCGG | |
| | CGGCGTGAGCGTGATCACCCCCGGCACCAACACCAGCAACCAGGTG | |
| | GCCGTGCTGTACCAGGGCGTGAACTGCACCGAGGTGCCCGTGGCC | |
| | ATCCACGCCGACCAGCTGACCCCCACCTGGAGGGTGTACAGCACCG | |
| | GCAGCAACGTGTTCCAGACCAGGGCCGGCTGCCTGATCGGCGCCG | |
| | AGTACGTGAACAACAGCTACGAGTGCGACATCCCCATCGGCGCCGG | |
| | CATCTGCGCCAGCTACCAGACCCAGACCAAGAGCCACCAGCAGGCC | |
| | CAGAGCGTGGCCAGCCAGAGCATCATCGCCTACACCATGAGCCTGG | |
| | GCGCCGAGAACAGCGTGGCCTACAGCAACAACAGCATCGCCATCCC | |
| | CACCAACTTCACCATCAGCGTGACCACCGAGATCCTGCCCGTGAGC | |
| | ATGACCAAGACCAGCGTGGACTGCACCATGTACATCTGCGGCGACA | |
| | GCACCGAGTGCAGCAACCTGCTGCTGCAGTACGGCAGCTTCTGCAC | |
| | CCAGCTGAAGAGGGCCCTGACCGGCATCGCCGTGGAGCAGGACAA | |
| | GAACACCCAGGAGGTGTTCGCCCAGGTGAAGCAGATCTACAAGACC | |
| | CCCCCCATCAAGTACTTCGGCGGCTTCAACTTCAGCCAGATCCTGCC | |
| | CGACCCCAGCAAGCCCAGCAAGAGGAGCCCCATCGAGGACCTGCTG | |
| | TTCAACAAGGTGACCCTGGCCGACGCCGGCTTCATCAAGCAGTACG | |
| | GCGACTGCCTGGGCGACATCGCCGCCAGGGACCTGATCTGCGCCC | |
| | AGAAGTTCAACGGCCTGACCGTGCTGCCCCCCCTGCTGACCGACGA | |
| | GATGATCGCCCAGTACACCAGCGCCCTGCTGGCCGGCACCATCACC | |
| | AGCGGCTGGACCTTCGGCGCCGGCCCCGCCCTGCAGATCCCCTTCC | |
| | CCATGCAGATGGCCTACAGGTTCAACGGCATCGGCGTGACCCAGAA | |
| | CGTGCTGTACGAGAACCAGAAGCTGATCGCCAACCAGTTCAACAGC | |
| | GCCATCGGCAAGATCCAGGACAGCCTGAGCAGCACCCCCAGCGCCC | |
| | TGGGCAAGCTGCAGGACGTGGTGAACCACAACGCCCAGGCCCTGAA | |
| | CACCCTGGTGAAGCAGCTGAGCAGCAAGTTCGGCGCCATCAGCAGC | |
| | GTGCTGAACGACATCCTGAGCAGGCTGGACCCCCCCGAGGCCGAG | |
| | GTGCAGATCGACAGGCTGATCACCGGCAGGCTGCAGAGCCTGCAGA | |
| | CCTACGTGACCCAGCAGCTGATCAGGGCCGCCGAGATCAGGGCCAG | |
| | CGCCAACCTGGCCGCCACCAAGATGAGCGAGTGCGTGCTGGGCCA | |
| | GAGCAAGAGGGTGGACTTCTGCGGCAAGGGCTACCACCTGATGAGC | |
| | TTCCCCCAGAGCGCCCCCCACGGCGTGGTGTTCCTGCACGTGACCT | |
| | ACGTGCCCGCCCAGGAGAAGAACTTCACCACCGCCCCCGCCATCTG | |
| | CCACGACGGCAAGGCCCACTTCCCCAGGGAGGGCGTGTTCGTGAG | |
| | CAACGGCACCCACTGGTTCGTGACCCAGAGGAACTTCTACGAGCCC | |
| | CAGATCATCACCACCGACAACACCTTCGTGAGCGGCAACTGCGACG | |
| | TGGTGATCGGCATCGTGAACAACACCGTGTACGACCCCCTGCAGCC | |
| | CGAGCTGGACAGCTTCAAGGAGGAGCTGGACAAGTACTTCAAGAAC | |
| | CACACCAGCCCCGACGTGGACCTGGGCGACATCAGCGGCATCAACG | |
| | CCAGCGTGGTGAACATCCAGAAGGAGATCGACAGGCTGAACGAGGT | |
| | GGCCAAGAACCTGAACGAGAGCCTGATCGACCTGCAGGAGCTGGGC | |
| | AAGTACGAGCAGTACATCAAGTGGCCCTGGTACATCTGGCTGGGCTT | |
| | CATCGCCGGCCTGATCGCCATCGTGATGGTGACCATCATGCTGTGCT | |
| | GCATGACCAGCTGCTGCAGCTGCCTGAAGGGCTGCTGCAGCTGCGG | |
| | CAGCTGCTGCAAGTTCGACGAGGACGACAGCGAGCCCGTGCTGAAG | |
| | GGCGTGAAGCTGCACTACACCGGAAGCGGAGCCACGAACTTCTCTC | |
| | TGTTAAAGCAAGCAGGAGATGTTGAAGAAAACCCCGGGCCTATGAAG | |
| | GCAATACTAGTAGTTCTGCTATATACATTTGCAACCGCAAATGCAGAC | |
| | ACATTATGTATAGGTTATCATGCGAACAACTCAACAGACACTGTAGAC | |
| | ACAGTACTAGAAAAGAATGTAACAGTAACACACTCTGTTAACCTTCTA | |
| | GAAGACAAGCATAACGGGAAACTATGCAAACTAAGAGGGGTAGCCC | |
| | CATTGCATTTGGGTAAATGTAACATTGCTGGCTGGATCCTGGGAAAT | |
| | CCAGAGTGTGAATCACTCTCCACAGCAAGCTCATGGTCCTACATTGT | |
| | GGAAACATCTAGTTCAGACAATGGAACGTGTTACCCAGGAGATTTCA | |
| | TCGATTATGAGGAGCTAAGAGAGCAATTGAGCTCAGTGTCATCATTT | |
| | GAAAGGTTTGAGATATTCCCCAAGACAAGTTCATGGCCCAATCATGA | |
| | CTCGAACAAAGGTGTAACGACAGCATGTCCTCATGCTGGAGCAAAAA | |
| | GCTTCTACAAAAATTTAATATGGCTAGTTAAAAAAGGAAATTCATACC | |
| | CAAAGCTCAGCAAATCCTACATTAATGATAAAGGGAAAGAAGTCCTC | |
| | GTGCTATGGGGCATTCACCATCCACCTACTAGTGCTGACCAACAAAG | |
| | TCTCTATCAGAATGCAGATGCATATGTTTTTGTGGGGACATCAAGATA | |
| | CAGCAAGAAGTTCAAGCCGGAAATAGCAATAAGGCCCAAAGTGAGG | |
| | GATCAAGAAGGGAGAATGAACTATTACTGGACACTAGTAGAGCCGGG | |
| | AGACAAAATAACATTCGAAGCAACTGGAAATCTAGTGGTACCGAGAT | |
| | ATGCATTCGCAATGGAAAGAAATGCTGGATCTGGTATTATCATTTCAG | |
| | ATACACCAGTCCACGATTGCAATACAACTTGTCAGACACCCAAGGGT | |
| | GCTATAAACACCAGCCTCCCATTTCAGAACATACATCCGATCACAATT | |
| | GGAAAATGTCCAAAATATGTAAAAAGCACAAAATTGAGACTGGCCAC | |
| | AGGATTGAGGAATGTCCCGTCCATTCAATCTAGAGGCCTATTTGGGG | |
| | CCATTGCCGGTTTCATTGAAGGGGGGTGGACAGGGATGGTAGATGG | |
| | ATGGTACGGTTATCACCATCAAAATGAGCAGGGGTCAGGATATGCAG | |
| | CCGACCTGAAGAGCACACAGAATGCCATTGACGAGATTACTAACAAA | |
| | GTAAACTCTGTTATTGAAAAAATGAATACACAGTTCACAGCAGTAGGT | |
| | AAAGAGTTCAACCACCTGGAAAAAAGAATAGAGAATTTAAATAAAAAA | |
| | GTTGATGATGGTTTCCTGGACATTTGGACTTACAATGCCGAACTGTTG | |
| | GTTCTATTGGAAAATGAAAGAACTTTGGACTACCACGATTCAAAGGTG | |
| | AAGAACTTATATGAAAAGGTAAGAAGCCAGTTAAAAAACAATGCCAAG | |
| | GAAATTGGAAACGGCTGCTTTGAATTTTACCACAAATGTGATAACACG | |
| | TGCATGGAAAGTGTCAAAAATGGGACTTATGACTACCCAAAATACTCA | |
| | GAGGAAGCAAAATTAAACAGAGAAGAAATAGATGGGGTAAAGCTGGA | |
| | ATCAACAAGGATTTACCAGATTTTGGCGATCTATTCAACCGTCGCCAG | |
| | TTCATTGGTACTGGTAGTCTCCCTGGGGGCAATCAGTTTCTGGATGT | |
| | GCTCTAATGGGTCTCTACAGTGTAGAATATGTATTTAAGGAAGCGGA | |
| | GCCACGAACTTCTCTCTGTTAAAGCAAGCAGGAGATGTTGAAGAAAA | |
| | CCCCGGGCCTAGCAAAAGCAGGGGATAATTCTATTAACCATGAAGAC | |
| | TATCATTGCTTTGAGCTACATTCTATGTCTGGTTTTCGCTCAAAAACTT | |
| | CCTGGAAATGACAATAGCACTGCAACGCTGTGCCTTGGGCACCATGC | |
| | AGTACCAAACGGAACGATAGTGAAAACAATCACGAATGACCAAATTG | |
| | AAGTTACTAATGCTACTGAGCTGGTTCAGAATTCCTCAATAGGTGAAA | |
| | TATGCGACAGTCCTCATCAGATCCTTGATGGAGAAAACTGCACACTA | |
| | ATAGATGCTCTATTGGGAGACCCTCAGTGTGATGGCTTTCAAAATAA | |
| | GAAATGGGACCTTTTTGTTGAACGAAGCAAAGCCTACAGCAACTGTT | |
| | ACCCTTATGATGTGCCGGATTATGCCTCCCTTAGGTCACTAGTTGCC | |
| | TCATCCGGCACACTGGAGTTTAACAATGAAAGCTTCAATTGGGCTGG | |
| | AGTCACTCAAAACGGAACAAGTTCTGCTTGCATAAGGGGATCTAATA | |
| | GTAGTTTCTTTAGTAGATTAAATTGGTTGACCCACTTAAACTTCAAGTA | |
| | CCCAGCATTGAACGTGACTATGCCAAACAATGAACAATTTGACAAATT | |
| | GTACATTTGGGGGGTTCACCACCCGGGTACGGACAAGGACCAAATC | |
| | TTCCTGTATGCTCAATCATCAGGAAGAATCACAGTATCTACCAAAAGA | |
| | AGCCAACAAGCTGTAATCCCGAATATCGGATCTAGACCCAGAATAAG | |
| | GAATATCCCTAGCAGAATAAGCATCTATTGGACAATAGTAAAACCGG | |
| | GAGACATACTTTTGATTAACAGCACAGGGAATCTAATTGCTCCTAGG | |
| | GGTTACTTCAAAATACGAAGTGGGAAAAGCTCAATAATGAGATCAGAT | |
| | GCACCCATTGGCAAATGCAAGTCTGAATGCATCACTCCAAATGGAAG | |
| | CATTCCCAATGACAAACCATTCCAAAATGTAAACAGGATCACATACGG | |
| | GGCCTGTCCCAGATATGTTAAGCAAAGCACTCTGAAATTGGCAACAG | |
| | GAATGCGAAATGTACCAGAGAAACAAACTAGAGGCATATTTGGCGCA | |
| | ATAGCGGGTTTCATAGAAAATGGTTGGGAGGGAATGGTGGATGGTTG | |
| | GTACGGTTTCAGGCATCAAAATTCTGAGGGAAGAGGACAAGCAGCA | |
| | GATCTCAAAAGCACTCAAGCAGCAATCGATCAAATCAATGGGAAGCT | |
| | GAATCGATTGATCGGGAAAACCAACGAGAAATTCCATCAGATTGAGA | |
| | AAGAATTCTCAGAAGTAGAAGGGAGAATTCAGGACCTTGAGAAATAT | |
| | GTTGAGGACACAAAAATAGATCTCTGGTCATACAACGCAGAGCTTCT | |
| | TGTTGCCCTGGAAAACCAACATACAATTGATCTAACTGACTCAGAAAT | |
| | GAACAAACTGTTTGAAAAAACAAAGAAGCAACTGAGGGAAAATGCTG | |
| | AGGATATGGGCAATGGTTGTTTCAAAATATACCACAAATGTGACAATG | |
| | CCTGCATAGGATCAATCAGAAATGGAACTTATGACCACGATGTATACA | |
| | GGGATGAAGCATTAAACAACCGGTTCCAGATCAAGGGAGTTGAGCTG | |
| | AAGTCAGGGTACAAAGATTGGATCCTATGGATTTCCTTTGCCATATCA | |
| | TGTTTTTTGCTTTGTGTTGCTTTGTTGGGGTTCATCATGTGGGCCTGC | |
| | CAAAAGGGCAACATTAGGTGCAACATTTGCATTTGAGTGCATTAATTA | |
| | AAAACACCCTTGTTTCTACTGGAAGCGGAGCCACGAACTTCTCTCTG | |
| | TTAAAGCAAGCAGGAGATGTTGAAGAAAACCCCGGGCCTATTTTCTA | |
| | ATATCCACAAAATGAAGGCAATAATTGTACTACTCATGGTAGTAACAT | |
| | CCAATGCAGATCGAATCTGCACTGGGATAACATCGTCAAACTCACCA | |
| | CATGTCGTCAAAACTGCTACTCAAGGGGAGGTCAACGTGACCGGTGT | |
| | AATACCACTGACAACAACACCCACCAAATCTCATTTTGCAAATCTCAA | |
| | AGGAACAGAAACCAGGGGGAAACTATGCCCAAAATGCCTCAACTGCA | |
| | CAGATCTGGATGTAGCCTTGGGCAGACCAAAATGCACAGGGAAAATA | |
| | CCCTCTGCAAGGGTTTCAATACTCCATGAAGTCAGACCTGTTACATCT | |
| | GGGTGCTTTCCTATAATGCACGATAGAACAAAAATTAGACAGCTGCC | |
| | TAACCTTCTCCGAGGATACGAACATGTCAGGTTATCAACTCACAACGT | |
| | TATCAATGCAAAAGATGCACCAGGAAGACCCTACGAAATTGGAACCT | |
| | CAGGGTCTTGCCCTAACATTACCAATGGAAACGGATTCTTCGCAACA | |
| | ATGGCTTGGGCCGTCCCAAAAAACAAAACAGCAACAAATCCATTAAC | |
| | AATAGAAGTACCATACATTTGTACAGAAGGAGAAGACCAAATTACCGT | |
| | TTGGGGGTTCCACTCTGACAACGAGACCCAAATGGCAAAGCTCTATG | |
| | GGGACTCAAAGCCCCAGAAGTTCACCTCATCTGCCAACGGAGTGAC | |
| | CACACATTACGTTTCACAGATTGGTGGCTTCCCAAATCAAACAGAAGA | |
| | CGGAGGACTACCACAAAGTGGCAGAATTGTTGTTGATTACATGGTGC | |
| | AGAAATCTGGAAAAACAGGAACAATTACCTATCAAAGAGGTATTTTAT | |
| | TGCCTCAAAAAGTGTGGTGCGCAAGTGGCAGGAGCAAGGTAATAAAA | |
| | GGATCCTTGCCCTTAATTGGAGAAGCAGATTGCCTCCATGAAAAATA | |
| | CGGTGGATTAAACAAAAGCAAGCCTTACTACACAGGGGAACATGCAA | |
| | AGGCCATAGGAAATTGCCCAATATGGGTGAAAACACCCTTGAAGCTG | |
| | GCCAATGGAACCAAATATAGACCCCCTGCAAAACTATTAAAGGAAAG | |
| | AGGTTTCTTCGGAGCCATTGCTGGTTTCTTAGAGGGAGGATGGGAAG | |
| | GAATGATTGCAGGTTGGCACGGATACACATCCCATGGGGCACATGG | |
| | AGTAGCGGTGGCAGCTGACCTTAAGAGCACTCAAGAGGCCATAAAC | |
| | AAGATAACAAAAAATCTCAACTCTTTGAGTGAGCTGGAAGTAAAGAAT | |
| | CTTCAAAGACTAAGCGGTGCCATGGATGAACTCCACAACGAAATACT | |
| | AGAACTAGATGAGAAAGTGGATGATCTCAGAGCTGACACAATAAGCT | |
| | CACAAATAGAACTCGCAGTCCTGCTTTCCAATGAAGGAATAATAAACA | |
| | GTGAAGATGAACATCTCTTGGCGCTTGAAAGAAAGCTGAAGAAAATG | |
| | CTGGGCCCCTCTGCTGTAGAGATAGGGAATGGATGCTTTGAAACCAA | |
| | ACACAAGTGCAACCAGACCTGCCTCGACAGAATAGCTGCTGGTACCT | |
| | TTGATGCAGGAGAATTTTCTCTCCCCACCTTTGATTCACTGAATATTA | |
| | CTGCTGCATCTTTAAATGACGACGGATTGGACAATCATACTATACTGC | |
| | TTTACTACTCAACTGCTGCCTCCAGTTTGGCTGTAACACTGATGATAG | |
| | CTATCTTTGTTGTTTATATGGTCTCCAGAGACAATGTTTCTTGCTCCAT | |
| | TTGTCTATAAGGAAAGTTAAGCCCTGTATTTTCCTTTATTGTAGTGCTT | |
| | GTTTGCTTGTTGTCATTACAAAGAAACGTTATTGAAAAATTGATGACT | |
| | CGAGCTGGTACTGCATGCACGCAATGCTAGCTGCCCCTTTCCCGTCC | |
| | TGGGTACCCCGAGTCTCCCCCGACCTCGGGTCCCAGGTATGCTCCC | |
| | ACCTCCACCTGCCCCACTCACCACCTCTGCTAGTTCCAGACACCTCC | |
| | CAAGCACGCAGCAATGCAGCTCAAAACGCTTAGCCTAGCCACACCC | |
| | CCACGGGAAACAGCAGTGATTAACCTTTAGCAATAAACGAAAGTTTAA | |
| | CTAAGCTATACTAACCCCAGGGTTGGTCAATTTCGTGCCAGCCACAC | |
| | CCTGGAGCTAGCAAAAAAAA | |
| Pan-CoV-Flu | TAGTTATTAATAGTAATCAATTACGGGGTCATTAGTTCATAGCCCATAT | 404 |
| Vaccine #16 | ATGGAGTTCCGCGTTACATAACTTACGGTAAATGGCCCGCCTGGCTG | |
| CoV-Omicron | ACCGCCCAACGACCCCCGCCCATTGACGTCAATAATGACGTATGTTC | |
| BA.2 Spike(2 | CCATAGTAACGCCAATAGGGACTTTCCATTGACGTCAATGGGTGGAG | |
| proline | TATTTACGGTAAACTGCCCACTTGGCAGTACATCAAGTGTATCATATG | |
| substitution)- | CCAAGTACGCCCCCTATTGACGTCAATGACGGTAAATGGCCCGCCTG | |
| Influenza (HA) | GCATTATGCCCAGTACATGACCTTATGGGACTTTCCTACTTGGCAGTA | |
| | CATCTACGTATTAGTCATCGCTATTACCATGGTGATGCGGTTTTGGCA | |
| | GTACATCAATGGGCGTGGATAGCGGTTTGACTCACGGGGATTTCCAA | |
| | GTCTCCACCCCATTGACGTCAATGGGAGTTTGTTTTGGCACCAAAAT | |
| | CAACGGGACTTTCCAAAATGTCGTAACAACTCCGCCCCATTGACGCA | |
| | AATGGGCGGTAGGCGTGTACGGTGGGAGGTCTATATAAGCAGAGCT | |
| | GGTTTAGTGAACCGTCAGATCGAGAATAAACTAGTATTCTTCTGGTCC | |
| | CCACAGACTCAGAGAGAACCCGCCACCATGTTCGTGTTCCTGGTGCT | |
| | GCTGCCCCTGGTGAGCAGCCAGTGCGTGAACCTGATCACCAGGACC | |
| | CAGAGCTACACCAACAGCTTCACCAGGGGCGTGTACTACCCCGACA | |
| | AGGTGTTCAGGAGCAGCGTGCTGCACAGCACCCAGGACCTGTTCCT | |
| | GCCCTTCTTCAGCAACGTGACCTGGTTCCACGCCATCCACGTGAGC | |
| | GGCACCAACGGCACCAAGAGGTTCGACAACCCCGTGCTGCCCTTCA | |
| | ACGACGGCGTGTACTTCGCCAGCACCGAGAAGAGCAACATCATCAG | |
| | GGGCTGGATCTTCGGCACCACCCTGGACAGCAAGACCCAGAGCCTG | |
| | CTGATCGTGAACAACGCCACCAACGTGGTGATCAAGGTGTGCGAGTT | |
| | CCAGTTCTGCAACGACCCCTTCCTGGACGTGTACTACCACAAGAACA | |
| | ACAAGAGCTGGATGGAGAGCGAGTTCAGGGTGTACAGCAGCGCCAA | |
| | CAACTGCACCTTCGAGTACGTGAGCCAGCCCTTCCTGATGGACCTG | |
| | GAGGGCAAGCAGGGCAACTTCAAGAACCTGAGGGAGTTCGTGTTCA | |
| | AGAACATCGACGGCTACTTCAAGATCTACAGCAAGCACACCCCCATC | |
| | AACCTGGGCAGGGACCTGCCCCAGGGCTTCAGCGCCCTGGAGCCC | |
| | CTGGTGGACCTGCCCATCGGCATCAACATCACCAGGTTCCAGACCCT | |
| | GCTGGCCCTGCACAGGAGCTACCTGACCCCCGGCGACAGCAGCAG | |
| | CGGCTGGACCGCCGGCGCCGCCGCCTACTACGTGGGCTACCTGCA | |
| | GCCCAGGACCTTCCTGCTGAAGTACAACGAGAACGGCACCATCACC | |
| | GACGCCGTGGACTGCGCCCTGGACCCCCTGAGCGAGACCAAGTGC | |
| | ACCCTGAAGAGCTTCACCGTGGAGAAGGGCATCTACCAGACCAGCA | |
| | ACTTCAGGGTGCAGCCCACCGAGAGCATCGTGAGGTTCCCCAACAT | |
| | CACCAACCTGTGCCCCTTCGACGAGGTGTTCAACGCCACCAGGTTC | |
| | GCCAGCGTGTACGCCTGGAACAGGAAGAGGATCAGCAACTGCGTGG | |
| | CCGACTACAGCGTGCTGTACAACTTCGCCCCCTTCTTCGCCTTCAAG | |
| | TGCTACGGCGTGAGCCCCACCAAGCTGAACGACCTGTGCTTCACCA | |
| | ACGTGTACGCCGACAGCTTCGTGATCAGGGGCAACGAGGTGAGCCA | |
| | GATCGCCCCCGGCCAGACCGGCAACATCGCCGACTACAACTACAAG | |
| | CTGCCCGACGACTTCACCGGCTGCGTGATCGCCTGGAACAGCAACA | |
| | AGCTGGACAGCAAGGTGGGCGGCAACTACAACTACCTGTACAGGCT | |
| | GTTCAGGAAGAGCAACCTGAAGCCCTTCGAGAGGGACATCAGCACC | |
| | GAGATCTACCAGGCCGGCAACAAGCCCTGCAACGGCGTGGCCGGCT | |
| | TCAACTGCTACTTCCCCCTGAGGAGCTACGGCTTCAGGCCCACCTAC | |
| | GGCGTGGGCCACCAGCCCTACAGGGTGGTGGTGCTGAGCTTCGAG | |
| | CTGCTGCACGCCCCCGCCACCGTGTGCGGCCCCAAGAAGAGCACCA | |
| | ACCTGGTGAAGAACAAGTGCGTGAACTTCAACTTCAACGGCCTGACC | |
| | GGCACCGGCGTGCTGACCGAGAGCAACAAGAAGTTCCTGCCCTTCC | |
| | AGCAGTTCGGCAGGGACATCGCCGACACCACCGACGCCGTGAGGG | |
| | ACCCCCAGACCCTGGAGATCCTGGACATCACCCCCTGCAGCTTCGG | |
| | CGGCGTGAGCGTGATCACCCCCGGCACCAACACCAGCAACCAGGTG | |
| | GCCGTGCTGTACCAGGGCGTGAACTGCACCGAGGTGCCCGTGGCC | |
| | ATCCACGCCGACCAGCTGACCCCCACCTGGAGGGTGTACAGCACCG | |
| | GCAGCAACGTGTTCCAGACCAGGGCCGGCTGCCTGATCGGCGCCG | |
| | AGTACGTGAACAACAGCTACGAGTGCGACATCCCCATCGGCGCCGG | |
| | CATCTGCGCCAGCTACCAGACCCAGACCAAGAGCCACCAGCAGGCC | |
| | CAGAGCGTGGCCAGCCAGAGCATCATCGCCTACACCATGAGCCTGG | |
| | GCGCCGAGAACAGCGTGGCCTACAGCAACAACAGCATCGCCATCCC | |
| | CACCAACTTCACCATCAGCGTGACCACCGAGATCCTGCCCGTGAGC | |
| | ATGACCAAGACCAGCGTGGACTGCACCATGTACATCTGCGGCGACA | |
| | GCACCGAGTGCAGCAACCTGCTGCTGCAGTACGGCAGCTTCTGCAC | |
| | CCAGCTGAAGAGGGCCCTGACCGGCATCGCCGTGGAGCAGGACAA | |
| | GAACACCCAGGAGGTGTTCGCCCAGGTGAAGCAGATCTACAAGACC | |
| | CCCCCCATCAAGTACTTCGGCGGCTTCAACTTCAGCCAGATCCTGCC | |
| | CGACCCCAGCAAGCCCAGCAAGAGGAGCTTCATCGAGGACCTGCTG | |
| | TTCAACAAGGTGACCCTGGCCGACGCCGGCTTCATCAAGCAGTACG | |
| | GCGACTGCCTGGGCGACATCGCCGCCAGGGACCTGATCTGCGCCC | |
| | AGAAGTTCAACGGCCTGACCGTGCTGCCCCCCCTGCTGACCGACGA | |
| | GATGATCGCCCAGTACACCAGCGCCCTGCTGGCCGGCACCATCACC | |
| | AGCGGCTGGACCTTCGGCGCCGGCGCCGCCCTGCAGATCCCCTTC | |
| | GCCATGCAGATGGCCTACAGGTTCAACGGCATCGGCGTGACCCAGA | |
| | ACGTGCTGTACGAGAACCAGAAGCTGATCGCCAACCAGTTCAACAGC | |
| | GCCATCGGCAAGATCCAGGACAGCCTGAGCAGCACCGCCAGCGCC | |
| | CTGGGCAAGCTGCAGGACGTGGTGAACCACAACGCCCAGGCCCTGA | |
| | ACACCCTGGTGAAGCAGCTGAGCAGCAAGTTCGGCGCCATCAGCAG | |
| | CGTGCTGAACGACATCCTGAGCAGGCTGGACCCCCCCGAGGCCGA | |
| | GGTGCAGATCGACAGGCTGATCACCGGCAGGCTGCAGAGCCTGCAG | |
| | ACCTACGTGACCCAGCAGCTGATCAGGGCCGCCGAGATCAGGGCCA | |
| | GCGCCAACCTGGCCGCCACCAAGATGAGCGAGTGCGTGCTGGGCC | |
| | AGAGCAAGAGGGTGGACTTCTGCGGCAAGGGCTACCACCTGATGAG | |
| | CTTCCCCCAGAGCGCCCCCCACGGCGTGGTGTTCCTGCACGTGACC | |
| | TACGTGCCCGCCCAGGAGAAGAACTTCACCACCGCCCCCGCCATCT | |
| | GCCACGACGGCAAGGCCCACTTCCCCAGGGAGGGCGTGTTCGTGA | |
| | GCAACGGCACCCACTGGTTCGTGACCCAGAGGAACTTCTACGAGCC | |
| | CCAGATCATCACCACCGACAACACCTTCGTGAGCGGCAACTGCGAC | |
| | GTGGTGATCGGCATCGTGAACAACACCGTGTACGACCCCCTGCAGC | |
| | CCGAGCTGGACAGCTTCAAGGAGGAGCTGGACAAGTACTTCAAGAA | |
| | CCACACCAGCCCCGACGTGGACCTGGGCGACATCAGCGGCATCAAC | |
| | GCCAGCGTGGTGAACATCCAGAAGGAGATCGACAGGCTGAACGAGG | |
| | TGGCCAAGAACCTGAACGAGAGCCTGATCGACCTGCAGGAGCTGGG | |
| | CAAGTACGAGCAGTACATCAAGTGGCCCTGGTACATCTGGCTGGGC | |
| | TTCATCGCCGGCCTGATCGCCATCGTGATGGTGACCATCATGCTGTG | |
| | CTGCATGACCAGCTGCTGCAGCTGCCTGAAGGGCTGCTGCAGCTGC | |
| | GGCAGCTGCTGCAAGTTCGACGAGGACGACAGCGAGCCCGTGCTGA | |
| | AGGGCGTGAAGCTGCACTACACCGGAAGCGGAGCCACGAACTTCTC | |
| | TCTGTTAAAGCAAGCAGGAGATGTTGAAGAAAACCCCGGGCCTATGA | |
| | AGGCAATACTAGTAGTTCTGCTATATACATTTGCAACCGCAAATGCAG | |
| | ACACATTATGTATAGGTTATCATGCGAACAACTCAACAGACACTGTAG | |
| | ACACAGTACTAGAAAAGAATGTAACAGTAACACACTCTGTTAACCTTC | |
| | TAGAAGACAAGCATAACGGGAAACTATGCAAACTAAGAGGGGTAGCC | |
| | CCATTGCATTTGGGTAAATGTAACATTGCTGGCTGGATCCTGGGAAA | |
| | TCCAGAGTGTGAATCACTCTCCACAGCAAGCTCATGGTCCTACATTG | |
| | TGGAAACATCTAGTTCAGACAATGGAACGTGTTACCCAGGAGATTTC | |
| | ATCGATTATGAGGAGCTAAGAGAGCAATTGAGCTCAGTGTCATCATTT | |
| | GAAAGGTTTGAGATATTCCCCAAGACAAGTTCATGGCCCAATCATGA | |
| | CTCGAACAAAGGTGTAACGACAGCATGTCCTCATGCTGGAGCAAAAA | |
| | GCTTCTACAAAAATTTAATATGGCTAGTTAAAAAAGGAAATTCATACC | |
| | CAAAGCTCAGCAAATCCTACATTAATGATAAAGGGAAAGAAGTCCTC | |
| | GTGCTATGGGGCATTCACCATCCACCTACTAGTGCTGACCAACAAAG | |
| | TCTCTATCAGAATGCAGATGCATATGTTTTTGTGGGGACATCAAGATA | |
| | CAGCAAGAAGTTCAAGCCGGAAATAGCAATAAGGCCCAAAGTGAGG | |
| | GATCAAGAAGGGAGAATGAACTATTACTGGACACTAGTAGAGCCGGG | |
| | AGACAAAATAACATTCGAAGCAACTGGAAATCTAGTGGTACCGAGAT | |
| | ATGCATTCGCAATGGAAAGAAATGCTGGATCTGGTATTATCATTTCAG | |
| | ATACACCAGTCCACGATTGCAATACAACTTGTCAGACACCCAAGGGT | |
| | GCTATAAACACCAGCCTCCCATTTCAGAACATACATCCGATCACAATT | |
| | GGAAAATGTCCAAAATATGTAAAAAGCAGAAAATTGAGACTGGCCAC | |
| | AGGATTGAGGAATGTCCCGTCCATTCAATCTAGAGGCCTATTTGGGG | |
| | CCATTGCCGGTTTCATTGAAGGGGGGTGGACAGGGATGGTAGATGG | |
| | ATGGTACGGTTATCACCATCAAAATGAGCAGGGGTCAGGATATGCAG | |
| | CCGACCTGAAGAGCACACAGAATGCCATTGACGAGATTACTAACAAA | |
| | GTAAACTCTGTTATTGAAAAAATGAATACACAGTTCACAGCAGTAGGT | |
| | AAAGAGTTCAACCACCTGGAAAAAAGAATAGAGAATTTAAATAAAAAA | |
| | GTTGATGATGGTTTCCTGGACATTTGGACTTACAATGCCGAACTGTTG | |
| | GTTCTATTGGAAAATGAAAGAACTTTGGACTACCACGATTCAAAGGTG | |
| | AAGAACTTATATGAAAAGGTAAGAAGCCAGTTAAAAAACAATGCCAAG | |
| | GAAATTGGAAACGGCTGCTTTGAATTTTACCACAAATGTGATAACACG | |
| | TGCATGGAAAGTGTCAAAAATGGGACTTATGACTACCCAAAATACTCA | |
| | GAGGAAGCAAAATTAAACAGAGAAGAAATAGATGGGGTAAAGCTGGA | |
| | ATCAACAAGGATTTACCAGATTTTGGCGATCTATTCAACCGTCGCCAG | |
| | TTCATTGGTACTGGTAGTCTCCCTGGGGGCAATCAGTTTCTGGATGT | |
| | GCTCTAATGGGTCTCTACAGTGTAGAATATGTATTTAAGGAAGCGGA | |
| | GCCACGAACTTCTCTCTGTTAAAGCAAGCAGGAGATGTTGAAGAAAA | |
| | CCCCGGGCCTAGCAAAAGCAGGGGATAATTCTATTAACCATGAAGAC | |
| | TATCATTGCTTTGAGCTACATTCTATGTCTGGTTTTCGCTCAAAAACTT | |
| | CCTGGAAATGACAATAGCACTGCAACGCTGTGCCTTGGGCACCATGC | |
| | AGTACCAAACGGAACGATAGTGAAAACAATCACGAATGACCAAATTG | |
| | AAGTTACTAATGCTACTGAGCTGGTTCAGAATTCCTCAATAGGTGAAA | |
| | TATGCGACAGTCCTCATCAGATCCTTGATGGAGAAAACTGCACACTA | |
| | ATAGATGCTCTATTGGGAGACCCTCAGTGTGATGGCTTTCAAAATAA | |
| | GAAATGGGACCTTTTTGTTGAACGAAGCAAAGCCTACAGCAACTGTT | |
| | ACCCTTATGATGTGCCGGATTATGCCTCCCTTAGGTCACTAGTTGCC | |
| | TCATCCGGCACACTGGAGTTTAACAATGAAAGCTTCAATTGGGCTGG | |
| | AGTCACTCAAAACGGAACAAGTTCTGCTTGCATAAGGGGATCTAATA | |
| | GTAGTTTCTTTAGTAGATTAAATTGGTTGACCCACTTAAACTTCAAGTA | |
| | CCCAGCATTGAACGTGACTATGCCAAACAATGAACAATTTGACAAATT | |
| | GTACATTTGGGGGGTTCACCACCCGGGTACGGACAAGGACCAAATC | |
| | TTCCTGTATGCTCAATCATCAGGAAGAATCACAGTATCTACCAAAAGA | |
| | AGCCAACAAGCTGTAATCCCGAATATCGGATCTAGACCCAGAATAAG | |
| | GAATATCCCTAGCAGAATAAGCATCTATTGGACAATAGTAAAACCGG | |
| | GAGACATACTTTGATTAACAGCACAGGGAATCTAATTGCTCCTAGG | |
| | GGTTACTTCAAAATACGAAGTGGGAAAAGCTCAATAATGAGATCAGAT | |
| | GCACCCATTGGCAAATGCAAGTCTGAATGCATCACTCCAAATGGAAG | |
| | CATTCCCAATGACAAACCATTCCAAAATGTAAACAGGATCACATACGG | |
| | GGCCTGTCCCAGATATGTTAAGCAAAGCACTCTGAAATTGGCAACAG | |
| | GAATGCGAAATGTACCAGAGAAACAAACTAGAGGCATATTTGGCGCA | |
| | ATAGCGGGTTTCATAGAAAATGGTTGGGAGGGAATGGTGGATGGTTG | |
| | GTACGGTTTCAGGCATCAAAATTCTGAGGGAAGAGGACAAGCAGCA | |
| | GATCTCAAAAGCACTCAAGCAGCAATCGATCAAATCAATGGGAAGCT | |
| | GAATCGATTGATCGGGAAAACCAACGAGAAATTCCATCAGATTGAGA | |
| | AAGAATTCTCAGAAGTAGAAGGGAGAATTCAGGACCTTGAGAAATAT | |
| | GTTGAGGACACAAAAATAGATCTCTGGTCATACAACGCAGAGCTTCT | |
| | TGTTGCCCTGGAAAACCAACATACAATTGATCTAACTGACTCAGAAAT | |
| | GAACAAACTGTTTGAAAAAACAAAGAAGCAACTGAGGGAAAATGCTG | |
| | AGGATATGGGCAATGGTTGTTTCAAAATATACCACAAATGTGACAATG | |
| | CCTGCATAGGATCAATCAGAAATGGAACTTATGACCACGATGTATACA | |
| | GGGATGAAGCATTAAACAACCGGTTCCAGATCAAGGGAGTTGAGCTG | |
| | AAGTCAGGGTACAAAGATTGGATCCTATGGATTTCCTTTGCCATATCA | |
| | TGTTTTTTGCTTTGTGTTGCTTTGTTGGGGTTCATCATGTGGGCCTGC | |
| | CAAAAGGGCAACATTAGGTGCAACATTTGCATTTGAGTGCATTAATTA | |
| | AAAACACCCTTGTTTCTACTGGAAGCGGAGCCACGAACTTCTCTCTG | |
| | TTAAAGCAAGCAGGAGATGTTGAAGAAAACCCCGGGCCTATTTTCTA | |
| | ATATCCACAAAATGAAGGCAATAATTGTACTACTCATGGTAGTAACAT | |
| | CCAATGCAGATCGAATCTGCACTGGGATAACATCGTCAAACTCACCA | |
| | CATGTCGTCAAAACTGCTACTCAAGGGGAGGTCAACGTGACCGGTGT | |
| | AATACCACTGACAACAACACCCACCAAATCTCATTTTGCAAATCTCAA | |
| | AGGAACAGAAACCAGGGGGAAACTATGCCCAAAATGCCTCAACTGCA | |
| | CAGATCTGGATGTAGCCTTGGGCAGACCAAAATGCACAGGGAAAATA | |
| | CCCTCTGCAAGGGTTTCAATACTCCATGAAGTCAGACCTGTTACATCT | |
| | GGGTGCTTTCCTATAATGCACGATAGAACAAAAATTAGACAGCTGCC | |
| | TAACCTTCTCCGAGGATACGAACATGTCAGGTTATCAACTCACAACGT | |
| | TATCAATGCAAAAGATGCACCAGGAAGACCCTACGAAATTGGAACCT | |
| | CAGGGTCTTGCCCTAACATTACCAATGGAAACGGATTCTTCGCAACA | |
| | ATGGCTTGGGCCGTCCCAAAAAACAAAACAGCAACAAATCCATTAAC | |
| | AATAGAAGTACCATACATTTGTACAGAAGGAGAAGACCAAATTACCGT | |
| | TTGGGGGTTCCACTCTGACAACGAGACCCAAATGGCAAAGCTCTATG | |
| | GGGACTCAAAGCCCCAGAAGTTCACCTCATCTGCCAACGGAGTGAC | |
| | CACACATTACGTTTCACAGATTGGTGGCTTCCCAAATCAAACAGAAGA | |
| | CGGAGGACTACCACAAAGTGGCAGAATTGTTGTTGATTACATGGTGC | |
| | AGAAATCTGGAAAAACAGGAACAATTACCTATCAAAGAGGTATTTTAT | |
| | TGCCTCAAAAAGTGTGGTGCGCAAGTGGCAGGAGCAAGGTAATAAAA | |
| | GGATCCTTGCCCTTAATTGGAGAAGCAGATTGCCTCCATGAAAAATA | |
| | CGGTGGATTAAACAAAAGCAAGCCTTACTACACAGGGGAACATGCAA | |
| | AGGCCATAGGAAATTGCCCAATATGGGTGAAAACACCCTTGAAGCTG | |
| | GCCAATGGAACCAAATATAGACCCCCTGCAAAACTATTAAAGGAAAG | |
| | AGGTTTCTTCGGAGCCATTGCTGGTTTCTTAGAGGGAGGATGGGAAG | |
| | GAATGATTGCAGGTTGGCACGGATACACATCCCATGGGGCACATGG | |
| | AGTAGCGGTGGCAGCTGACCTTAAGAGCACTCAAGAGGCCATAAAC | |
| | AAGATAACAAAAAATCTCAACTCTTTGAGTGAGCTGGAAGTAAAGAAT | |
| | CTTCAAAGACTAAGCGGTGCCATGGATGAACTCCACAACGAAATACT | |
| | AGAACTAGATGAGAAAGTGGATGATCTCAGAGCTGACACAATAAGCT | |
| | CACAAATAGAACTCGCAGTCCTGCTTTCCAATGAAGGAATAATAAACA | |
| | GTGAAGATGAACATCTCTTGGCGCTTGAAAGAAAGCTGAAGAAAATG | |
| | CTGGGCCCCTCTGCTGTAGAGATAGGGAATGGATGCTTTGAAACCAA | |
| | ACACAAGTGCAACCAGACCTGCCTCGACAGAATAGCTGCTGGTACCT | |
| | TTGATGCAGGAGAATTTTCTCTCCCCACCTTTGATTCACTGAATATTA | |
| | CTGCTGCATCTTTAAATGACGACGGATTGGACAATCATACTATACTGC | |
| | TTTACTACTCAACTGCTGCCTCCAGTTTGGCTGTAACACTGATGATAG | |
| | CTATCTTTGTTGTTTATATGGTCTCCAGAGACAATGTTTCTTGCTCCAT | |
| | TTGTCTATAAGGAAAGTTAAGCCCTGTATTTTCCTTTATTGTAGTGCTT | |
| | GTTTGCTTGTTGTCATTACAAAGAAACGTTATTGAAAAATTGATGACT | |
| | CGAGCTGGTACTGCATGCACGCAATGCTAGCTGCCCCTTTCCCGTCC | |
| | TGGGTACCCCGAGTCTCCCCCGACCTCGGGTCCCAGGTATGCTCCC | |
| | ACCTCCACCTGCCCCACTCACCACCTCTGCTAGTTCCAGACACCTCC | |
| | CAAGCACGCAGCAATGCAGCTCAAAACGCTTAGCCTAGCCACACCC | |
| | CCACGGGAAACAGCAGTGATTAACCTTTAGCAATAAACGAAAGTTTAA | |
| | CTAAGCTATACTAACCCCAGGGTTGGTCAATTTCGTGCCAGCCACAC | |
| | CCTGGAGCTAGCAAAAAAAA | |
| Pan-CoV-Flu | CTCGACATTGATTATTGACTAGTTATTAATAGTAATCAATTACGGGGT | 405 |
| Vaccine #17 | CATTAGTTCATAGCCCATATATGGAGTTCCGCGTTACATAACTTACGG | |
| CoV- Omicron | TAAATGGCCCGCCTGGCTGACCGCCCAACGACCCCCGCCCATTGAC | |
| BA.2 | GTCAATAATGACGTATGTTCCCATAGTAACGCCAATAGGGACTTTCCA | |
| Spike(HexaPro)- | TTGACGTCAATGGGTGGAGTATTTACGGTAAACTGCCCACTTGGCAG | |
| Influenza (HA) | TACATCAAGTGTATCATATGCCAAGTACGCCCCCTATTGACGTCAATG | |
| | ACGGTAAATGGCCCGCCTGGCATTATGCCCAGTACATGACCTTATGG | |
| | GACTTTCCTACTTGGCAGTACATCTACGTATTAGTCATCGCTATTACC | |
| | ATGGTCGAGGTGAGCCCCACGTTCTGCTTCACTCTCCCCATCTCCCC | |
| | CCCCTCCCCACCCCCAATTTTGTATTTATTTATTTTTTAATTATTTTGT | |
| | GCAGCGATGGGGGCGGGGGGGGGGGGGGGGCGCGCGCCAGGCG | |
| | GGGCGGGGCGGGGCGAGGGGCGGGGCGGGGCGAGGCGGAGAGG | |
| | TGCGGCGGCAGCCAATCAGAGCGGCGCGCTCCGAAAGTTTCCTTTT | |
| | ATGGCGAGGCGGCGGCGGCGGCGGCCCTATAAAAAGCGAAGCGCG | |
| | CGGCGGGCGGGAGTCGCTGCGCGCTGCCTTCGCCCCGTGCCCCGC | |
| | TCCGCCGCCGCCTCGCGCCGCCCGCCCCGGCTCTGACTGACCGCG | |
| | TTACTCCCACAGGTGAGCGGGCGGGACGGCCCTTCTCCTCCGGGCT | |
| | GTAATTAGCGCTTGGTTTAATGACGGCTTGTTTCTTTTCTGTGGCTGC | |
| | GTGAAAGCCTTGAGGGGCTCCGGGAGGGCCCTTTGTGCGGGGGGA | |
| | GCGGCTCGGGGGGTGCGTGCGTGTGTGTGTGCGTGGGGAGCGCCG | |
| | CGTGCGGCTCCGCGCTGCCCGGCGGCTGTGAGCGCTGCGGGCGCG | |
| | GCGCGGGGCTTTGTGCGCTCCGCAGTGTGCGCGAGGGGAGCGCGG | |
| | CCGGGGGCGGTGCCCCGCGGTGCGGGGGGGGCTGCGAGGGGAAC | |
| | AAAGGCTGCGTGCGGGGTGTGTGCGTGGGGGGGTGAGCAGGGGGT | |
| | GTGGGCGCGTCGGTCGGGCTGCAACCCCCCCTGCACCCCCCTCCC | |
| | CGAGTTGCTGAGCACGGCCCGGCTTCGGGTGCGGGGCTCCGTACG | |
| | GGGCGTGGCGCGGGGCTCGCCGTGCCGGGCGGGGGGTGGCGGCA | |
| | GGTGGGGGTGCCGGGCGGGGCGGGGCCGCCTCGGGCCGGGGAG | |
| | GGCTCGGGGGAGGGGCGCGGCGGCCCCCGGAGCGCCGGCGGCTG | |
| | TCGAGGCGCGGCGAGCCGCAGCCATTGCCTTTTATGGTAATCGTGC | |
| | GAGAGGGCGCAGGGACTTCCTTTGTCCCAAATCTGTGCGGAGCCGA | |
| | AATCTGGGAGGCGCCGCCGCACCCCCTCTAGCGGGCGCGGGGCGA | |
| | AGCGGTGCGGCGCCGGCAGGAAGGAAATGGGCGGGGAGGGCCTTC | |
| | GTGCGTCGCCGCGCCGCCGTCCCCTTCTCCCTCTCCAGCCTCGGGG | |
| | CTGTCCGCGGGGGGACGGCTGCCTTCGGGGGGGACGGGGCAGGG | |
| | CGGGGTTCGGCTTCTGGCGTGTGACCGGCGGCTCTAGAGCCTCTGC | |
| | TAACCATGTTCATGCCTTCTTCTTTTTCCTACAGCTCCTGGGCAACGT | |
| | GCTGGTTATTGTGCTGTCTCATCATTTTGGCAAAGAATTGGAGAATAA | |
| | ACTAGTATTCTTCTGGTCCCCACAGACTCAGAGAGAACCCGCCACCA | |
| | TGTTCGTGTTCCTGGTGCTGCTGCCCCTGGTGAGCAGCCAGTGCGT | |
| | GAACCTGATCACCAGGACCCAGAGCTACACCAACAGCTTCACCAGG | |
| | GGCGTGTACTACCCCGACAAGGTGTTCAGGAGCAGCGTGCTGCACA | |
| | GCACCCAGGACCTGTTCCTGCCCTTCTTCAGCAACGTGACCTGGTTC | |
| | CACGCCATCCACGTGAGCGGCACCAACGGCACCAAGAGGTTCGACA | |
| | ACCCCGTGCTGCCCTTCAACGACGGCGTGTACTTCGCCAGCACCGA | |
| | GAAGAGCAACATCATCAGGGGCTGGATCTTCGGCACCACCCTGGAC | |
| | AGCAAGACCCAGAGCCTGCTGATCGTGAACAACGCCACCAACGTGG | |
| | TGATCAAGGTGTGCGAGTTCCAGTTCTGCAACGACCCCTTCCTGGAC | |
| | GTGTACTACCACAAGAACAACAAGAGCTGGATGGAGAGCGAGTTCA | |
| | GGGTGTACAGCAGCGCCAACAACTGCACCTTCGAGTACGTGAGCCA | |
| | GCCCTTCCTGATGGACCTGGAGGGCAAGCAGGGCAACTTCAAGAAC | |
| | CTGAGGGAGTTCGTGTTCAAGAACATCGACGGCTACTTCAAGATCTA | |
| | CAGCAAGCACACCCCCATCAACCTGGGCAGGGACCTGCCCCAGGGC | |
| | TTCAGCGCCCTGGAGCCCCTGGTGGACCTGCCCATCGGCATCAACA | |
| | TCACCAGGTTCCAGACCCTGCTGGCCCTGCACAGGAGCTACCTGAC | |
| | CCCCGGCGACAGCAGCAGCGGCTGGACCGCCGGCGCCGCCGCCTA | |
| | CTACGTGGGCTACCTGCAGCCCAGGACCTTCCTGCTGAAGTACAAC | |
| | GAGAACGGCACCATCACCGACGCCGTGGACTGCGCCCTGGACCCC | |
| | CTGAGCGAGACCAAGTGCACCCTGAAGAGCTTCACCGTGGAGAAGG | |
| | GCATCTACCAGACCAGCAACTTCAGGGTGCAGCCCACCGAGAGCAT | |
| | CGTGAGGTTCCCCAACATCACCAACCTGTGCCCCTTCGACGAGGTGT | |
| | TCAACGCCACCAGGTTCGCCAGCGTGTACGCCTGGAACAGGAAGAG | |
| | GATCAGCAACTGCGTGGCCGACTACAGCGTGCTGTACAACTTCGCC | |
| | CCCTTCTTCGCCTTCAAGTGCTACGGCGTGAGCCCCACCAAGCTGAA | |
| | CGACCTGTGCTTCACCAACGTGTACGCCGACAGCTTCGTGATCAGG | |
| | GGCAACGAGGTGAGCCAGATCGCCCCCGGCCAGACCGGCAACATC | |
| | GCCGACTACAACTACAAGCTGCCCGACGACTTCACCGGCTGCGTGA | |
| | TCGCCTGGAACAGCAACAAGCTGGACAGCAAGGTGGGCGGCAACTA | |
| | CAACTACCTGTACAGGCTGTTCAGGAAGAGCAACCTGAAGCCCTTCG | |
| | AGAGGGACATCAGCACCGAGATCTACCAGGCCGGCAACAAGCCCTG | |
| | CAACGGCGTGGCCGGCTTCAACTGCTACTTCCCCCTGAGGAGCTAC | |
| | GGCTTCAGGCCCACCTACGGCGTGGGCCACCAGCCCTACAGGGTG | |
| | GTGGTGCTGAGCTTCGAGCTGCTGCACGCCCCCGCCACCGTGTGCG | |
| | GCCCCAAGAAGAGCACCAACCTGGTGAAGAACAAGTGCGTGAACTT | |
| | CAACTTCAACGGCCTGACCGGCACCGGCGTGCTGACCGAGAGCAAC | |
| | AAGAAGTTCCTGCCCTTCCAGCAGTTCGGCAGGGACATCGCCGACA | |
| | CCACCGACGCCGTGAGGGACCCCCAGACCCTGGAGATCCTGGACAT | |
| | CACCCCCTGCAGCTTCGGCGGCGTGAGCGTGATCACCCCCGGCACC | |
| | AACACCAGCAACCAGGTGGCCGTGCTGTACCAGGGCGTGAACTGCA | |
| | CCGAGGTGCCCGTGGCCATCCACGCCGACCAGCTGACCCCCACCTG | |
| | GAGGGTGTACAGCACCGGCAGCAACGTGTTCCAGACCAGGGCCGG | |
| | CTGCCTGATCGGCGCCGAGTACGTGAACAACAGCTACGAGTGCGAC | |
| | ATCCCCATCGGCGCCGGCATCTGCGCCAGCTACCAGACCCAGACCA | |
| | AGAGCCACCAGCAGGCCCAGAGCGTGGCCAGCCAGAGCATCATCG | |
| | CCTACACCATGAGCCTGGGCGCCGAGAACAGCGTGGCCTACAGCAA | |
| | CAACAGCATCGCCATCCCCACCAACTTCACCATCAGCGTGACCACCG | |
| | AGATCCTGCCCGTGAGCATGACCAAGACCAGCGTGGACTGCACCAT | |
| | GTACATCTGCGGCGACAGCACCGAGTGCAGCAACCTGCTGCTGCAG | |
| | TACGGCAGCTTCTGCACCCAGCTGAAGAGGGCCCTGACCGGCATCG | |
| | CCGTGGAGCAGGACAAGAACACCCAGGAGGTGTTCGCCCAGGTGAA | |
| | GCAGATCTACAAGACCCCCCCCATCAAGTACTTCGGCGGCTTCAACT | |
| | TCAGCCAGATCCTGCCCGACCCCAGCAAGCCCAGCAAGAGGAGCCC | |
| | CATCGAGGACCTGCTGTTCAACAAGGTGACCCTGGCCGACGCCGGC | |
| | TTCATCAAGCAGTACGGCGACTGCCTGGGCGACATCGCCGCCAGGG | |
| | ACCTGATCTGCGCCCAGAAGTTCAACGGCCTGACCGTGCTGCCCCC | |
| | CCTGCTGACCGACGAGATGATCGCCCAGTACACCAGCGCCCTGCTG | |
| | GCCGGCACCATCACCAGCGGCTGGACCTTCGGCGCCGGCCCCGCC | |
| | CTGCAGATCCCCTTCCCCATGCAGATGGCCTACAGGTTCAACGGCAT | |
| | CGGCGTGACCCAGAACGTGCTGTACGAGAACCAGAAGCTGATCGCC | |
| | AACCAGTTCAACAGCGCCATCGGCAAGATCCAGGACAGCCTGAGCA | |
| | GCACCCCCAGCGCCCTGGGCAAGCTGCAGGACGTGGTGAACCACAA | |
| | CGCCCAGGCCCTGAACACCCTGGTGAAGCAGCTGAGCAGCAAGTTC | |
| | GGCGCCATCAGCAGCGTGCTGAACGACATCCTGAGCAGGCTGGACC | |
| | CCCCCGAGGCCGAGGTGCAGATCGACAGGCTGATCACCGGCAGGC | |
| | TGCAGAGCCTGCAGACCTACGTGACCCAGCAGCTGATCAGGGCCGC | |
| | CGAGATCAGGGCCAGCGCCAACCTGGCCGCCACCAAGATGAGCGA | |
| | GTGCGTGCTGGGCCAGAGCAAGAGGGTGGACTTCTGCGGCAAGGG | |
| | CTACCACCTGATGAGCTTCCCCCAGAGCGCCCCCCACGGCGTGGTG | |
| | TTCCTGCACGTGACCTACGTGCCCGCCCAGGAGAAGAACTTCACCA | |
| | CCGCCCCCGCCATCTGCCACGACGGCAAGGCCCACTTCCCCAGGGA | |
| | GGGCGTGTTCGTGAGCAACGGCACCCACTGGTTCGTGACCCAGAGG | |
| | AACTTCTACGAGCCCCAGATCATCACCACCGACAACACCTTCGTGAG | |
| | CGGCAACTGCGACGTGGTGATCGGCATCGTGAACAACACCGTGTAC | |
| | GACCCCCTGCAGCCCGAGCTGGACAGCTTCAAGGAGGAGCTGGACA | |
| | AGTACTTCAAGAACCACACCAGCCCCGACGTGGACCTGGGCGACAT | |
| | CAGCGGCATCAACGCCAGCGTGGTGAACATCCAGAAGGAGATCGAC | |
| | AGGCTGAACGAGGTGGCCAAGAACCTGAACGAGAGCCTGATCGACC | |
| | TGCAGGAGCTGGGCAAGTACGAGCAGTACATCAAGTGGCCCTGGTA | |
| | CATCTGGCTGGGCTTCATCGCCGGCCTGATCGCCATCGTGATGGTG | |
| | ACCATCATGCTGTGCTGCATGACCAGCTGCTGCAGCTGCCTGAAGG | |
| | GCTGCTGCAGCTGCGGCAGCTGCTGCAAGTTCGACGAGGACGACAG | |
| | CGAGCCCGTGCTGAAGGGCGTGAAGCTGCACTACACCGGAAGCGG | |
| | AGCCACGAACTTCTCTCTGTTAAAGCAAGCAGGAGATGTTGAAGAAA | |
| | ACCCCGGGCCTATGAAGGCAATACTAGTAGTTCTGCTATATACATTTG | |
| | CAACCGCAAATGCAGACACATTATGTATAGGTTATCATGCGAACAACT | |
| | CAACAGACACTGTAGACACAGTACTAGAAAAGAATGTAACAGTAACA | |
| | CACTCTGTTAACCTTCTAGAAGACAAGCATAACGGGAAACTATGCAAA | |
| | CTAAGAGGGGTAGCCCCATTGCATTTGGGTAAATGTAACATTGCTGG | |
| | CTGGATCCTGGGAAATCCAGAGTGTGAATCACTCTCCACAGCAAGCT | |
| | CATGGTCCTACATTGTGGAAACATCTAGTTCAGACAATGGAACGTGTT | |
| | ACCCAGGAGATTTCATCGATTATGAGGAGCTAAGAGAGCAATTGAGC | |
| | TCAGTGTCATCATTTGAAAGGTTTGAGATATTCCCCAAGACAAGTTCA | |
| | TGGCCCAATCATGACTCGAACAAAGGTGTAACGACAGCATGTCCTCA | |
| | TGCTGGAGCAAAAAGCTTCTACAAAAATTTAATATGGCTAGTTAAAAA | |
| | AGGAAATTCATACCCAAAGCTCAGCAAATCCTACATTAATGATAAAGG | |
| | GAAAGAAGTCCTCGTGCTATGGGGCATTCACCATCCACCTACTAGTG | |
| | CTGACCAACAAAGTCTCTATCAGAATGCAGATGCATATGTTTTTGTGG | |
| | GGACATCAAGATACAGCAAGAAGTTCAAGCCGGAAATAGCAATAAGG | |
| | CCCAAAGTGAGGGATCAAGAAGGGAGAATGAACTATTACTGGACACT | |
| | AGTAGAGCCGGGAGACAAAATAACATTCGAAGCAACTGGAAATCTAG | |
| | TGGTACCGAGATATGCATTCGCAATGGAAAGAAATGCTGGATCTGGT | |
| | ATTATCATTTCAGATACACCAGTCCACGATTGCAATACAACTTGTCAG | |
| | ACACCCAAGGGTGCTATAAACACCAGCCTCCCATTTCAGAACATACA | |
| | TCCGATCACAATTGGAAAATGTCCAAAATATGTAAAAAGCACAAAATT | |
| | GAGACTGGCCACAGGATTGAGGAATGTCCCGTCCATTCAATCTAGAG | |
| | GCCTATTTGGGGCCATTGCCGGTTTCATTGAAGGGGGGTGGACAGG | |
| | GATGGTAGATGGATGGTACGGTTATCACCATCAAAATGAGCAGGGGT | |
| | CAGGATATGCAGCCGACCTGAAGAGCACACAGAATGCCATTGACGA | |
| | GATTACTAACAAAGTAAACTCTGTTATTGAAAAAATGAATACACAGTTC | |
| | ACAGCAGTAGGTAAAGAGTTCAACCACCTGGAAAAAAGAATAGAGAA | |
| | TTTAAATAAAAAAGTTGATGATGGTTTCCTGGACATTTGGACTTACAAT | |
| | GCCGAACTGTTGGTTCTATTGGAAAATGAAAGAACTTTGGACTACCA | |
| | CGATTCAAAGGTGAAGAACTTATATGAAAAGGTAAGAAGCCAGTTAAA | |
| | AAACAATGCCAAGGAAATTGGAAACGGCTGCTTTGAATTTTACCACAA | |
| | ATGTGATAACACGTGCATGGAAAGTGTCAAAAATGGGACTTATGACT | |
| | ACCCAAAATACTCAGAGGAAGCAAAATTAAACAGAGAAGAAATAGAT | |
| | GGGGTAAAGCTGGAATCAACAAGGATTTACCAGATTTTGGCGATCTA | |
| | TTCAACCGTCGCCAGTTCATTGGTACTGGTAGTCTCCCTGGGGGCAA | |
| | TCAGTTTCTGGATGTGCTCTAATGGGTCTCTACAGTGTAGAATATGTA | |
| | TTTAAGGAAGCGGAGCCACGAACTTCTCTCTGTTAAAGCAAGCAGGA | |
| | GATGTTGAAGAAAACCCCGGGCCTAGCAAAAGCAGGGGATAATTCTA | |
| | TTAACCATGAAGACTATCATTGCTTTGAGCTACATTCTATGTCTGGTTT | |
| | TCGCTCAAAAACTTCCTGGAAATGACAATAGCACTGCAACGCTGTGC | |
| | CTTGGGCACCATGCAGTACCAAACGGAACGATAGTGAAAACAATCAC | |
| | GAATGACCAAATTGAAGTTACTAATGCTACTGAGCTGGTTCAGAATTC | |
| | CTCAATAGGTGAAATATGCGACAGTCCTCATCAGATCCTTGATGGAG | |
| | AAAACTGCACACTAATAGATGCTCTATTGGGAGACCCTCAGTGTGAT | |
| | GGCTTTCAAAATAAGAAATGGGACCTTTTTGTTGAACGAAGCAAAGC | |
| | CTACAGCAACTGTTACCCTTATGATGTGCCGGATTATGCCTCCCTTAG | |
| | GTCACTAGTTGCCTCATCCGGCACACTGGAGTTTAACAATGAAAGCT | |
| | TCAATTGGGCTGGAGTCACTCAAAACGGAACAAGTTCTGCTTGCATA | |
| | AGGGGATCTAATAGTAGTTTCTTTAGTAGATTAAATTGGTTGACCCAC | |
| | TTAAACTTCAAGTACCCAGCATTGAACGTGACTATGCCAAACAATGAA | |
| | CAATTTGACAAATTGTACATTTGGGGGGTTCACCACCCGGGTACGGA | |
| | CAAGGACCAAATCTTCCTGTATGCTCAATCATCAGGAAGAATCACAGT | |
| | ATCTACCAAAAGAAGCCAACAAGCTGTAATCCCGAATATCGGATCTA | |
| | GACCCAGAATAAGGAATATCCCTAGCAGAATAAGCATCTATTGGACA | |
| | ATAGTAAAACCGGGAGACATACTTTTGATTAACAGCACAGGGAATCTA | |
| | ATTGCTCCTAGGGGTTACTTCAAAATACGAAGTGGGAAAAGCTCAAT | |
| | AATGAGATCAGATGCACCCATTGGCAAATGCAAGTCTGAATGCATCA | |
| | CTCCAAATGGAAGCATTCCCAATGACAAACCATTCCAAAATGTAAACA | |
| | GGATCACATACGGGGCCTGTCCCAGATATGTTAAGCAAAGCACTCTG | |
| | AAATTGGCAACAGGAATGCGAAATGTACCAGAGAAACAAACTAGAGG | |
| | CATATTTGGCGCAATAGCGGGTTTCATAGAAAATGGTTGGGAGGGAA | |
| | TGGTGGATGGTTGGTACGGTTTCAGGCATCAAAATTCTGAGGGAAGA | |
| | GGACAAGCAGCAGATCTCAAAAGCACTCAAGCAGCAATCGATCAAAT | |
| | CAATGGGAAGCTGAATCGATTGATCGGGAAAACCAACGAGAAATTCC | |
| | ATCAGATTGAGAAAGAATTCTCAGAAGTAGAAGGGAGAATTCAGGAC | |
| | CTTGAGAAATATGTTGAGGACACAAAAATAGATCTCTGGTCATACAAC | |
| | GCAGAGCTTCTTGTTGCCCTGGAAAACCAACATACAATTGATCTAACT | |
| | GACTCAGAAATGAACAAACTGTTTGAAAAAACAAAGAAGCAACTGAG | |
| | GGAAAATGCTGAGGATATGGGCAATGGTTGTTTCAAAATATACCACA | |
| | AATGTGACAATGCCTGCATAGGATCAATCAGAAATGGAACTTATGAC | |
| | CACGATGTATACAGGGATGAAGCATTAAACAACCGGTTCCAGATCAA | |
| | GGGAGTTGAGCTGAAGTCAGGGTACAAAGATTGGATCCTATGGATTT | |
| | CCTTTGCCATATCATGTTTTTTGCTTTGTGTTGCTTTGTTGGGGTTCAT | |
| | CATGTGGGCCTGCCAAAAGGGCAACATTAGGTGCAACATTTGCATTT | |
| | GAGTGCATTAATTAAAAACACCCTTGTTTCTACTGGAAGCGGAGCCA | |
| | CGAACTTCTCTCTGTTAAAGCAAGCAGGAGATGTTGAAGAAAACCCC | |
| | GGGCCTATTTTCTAATATCCACAAAATGAAGGCAATAATTGTACTACT | |
| | CATGGTAGTAACATCCAATGCAGATCGAATCTGCACTGGGATAACAT | |
| | CGTCAAACTCACCACATGTCGTCAAAACTGCTACTCAAGGGGAGGTC | |
| | AACGTGACCGGTGTAATACCACTGACAACAACACCCACCAAATCTCA | |
| | TTTTGCAAATCTCAAAGGAACAGAAACCAGGGGGAAACTATGCCCAA | |
| | AATGCCTCAACTGCACAGATCTGGATGTAGCCTTGGGCAGACCAAAA | |
| | TGCACAGGGAAAATACCCTCTGCAAGGGTTTCAATACTCCATGAAGT | |
| | CAGACCTGTTACATCTGGGTGCTTTCCTATAATGCACGATAGAACAAA | |
| | AATTAGACAGCTGCCTAACCTTCTCCGAGGATACGAACATGTCAGGT | |
| | TATCAACTCACAACGTTATCAATGCAAAAGATGCACCAGGAAGACCC | |
| | TACGAAATTGGAACCTCAGGGTCTTGCCCTAACATTACCAATGGAAA | |
| | CGGATTCTTCGCAACAATGGCTTGGGCCGTCCCAAAAAACAAAACAG | |
| | CAACAAATCCATTAACAATAGAAGTACCATACATTTGTACAGAAGGAG | |
| | AAGACCAAATTACCGTTTGGGGGTTCCACTCTGACAACGAGACCCAA | |
| | ATGGCAAAGCTCTATGGGGACTCAAAGCCCCAGAAGTTCACCTCATC | |
| | TGCCAACGGAGTGACCACACATTACGTTTCACAGATTGGTGGCTTCC | |
| | CAAATCAAACAGAAGACGGAGGACTACCACAAAGTGGCAGAATTGTT | |
| | GTTGATTACATGGTGCAGAAATCTGGAAAAACAGGAACAATTACCTAT | |
| | CAAAGAGGTATTTTATTGCCTCAAAAAGTGTGGTGCGCAAGTGGCAG | |
| | GAGCAAGGTAATAAAAGGATCCTTGCCCTTAATTGGAGAAGCAGATT | |
| | GCCTCCATGAAAAATACGGTGGATTAAACAAAAGCAAGCCTTACTAC | |
| | ACAGGGGAACATGCAAAGGCCATAGGAAATTGCCCAATATGGGTGAA | |
| | AACACCCTTGAAGCTGGCCAATGGAACCAAATATAGACCCCCTGCAA | |
| | AACTATTAAAGGAAAGAGGTTTCTTCGGAGCCATTGCTGGTTTCTTAG | |
| | AGGGAGGATGGGAAGGAATGATTGCAGGTTGGCACGGATACACATC | |
| | CCATGGGGCACATGGAGTAGCGGTGGCAGCTGACCTTAAGAGCACT | |
| | CAAGAGGCCATAAACAAGATAACAAAAAATCTCAACTCTTTGAGTGAG | |
| | CTGGAAGTAAAGAATCTTCAAAGACTAAGCGGTGCCATGGATGAACT | |
| | CCACAACGAAATACTAGAACTAGATGAGAAAGTGGATGATCTCAGAG | |
| | CTGACACAATAAGCTCACAAATAGAACTCGCAGTCCTGCTTTCCAATG | |
| | AAGGAATAATAAACAGTGAAGATGAACATCTCTTGGCGCTTGAAAGA | |
| | AAGCTGAAGAAAATGCTGGGCCCCTCTGCTGTAGAGATAGGGAATG | |
| | GATGCTTTGAAACCAAACACAAGTGCAACCAGACCTGCCTCGACAGA | |
| | ATAGCTGCTGGTACCTTTGATGCAGGAGAATTTTCTCTCCCCACCTTT | |
| | GATTCACTGAATATTACTGCTGCATCTTTAAATGACGACGGATTGGAC | |
| | AATCATACTATACTGCTTTACTACTCAACTGCTGCCTCCAGTTTGGCT | |
| | GTAACACTGATGATAGCTATCTTTGTTGTTTATATGGTCTCCAGAGAC | |
| | AATGTTTCTTGCTCCATTTGTCTATAAGGAAAGTTAAGCCCTGTATTTT | |
| | CCTTTATTGTAGTGCTTGTTTGCTTGTTGTCATTACAAAGAAACGTTAT | |
| | TGAAAAATTGATGACTCGAGCTGGTACTGCATGCACGCAATGCTAGC | |
| | TGCCCCTTTCCCGTCCTGGGTACCCCGAGTCTCCCCCGACCTCGGG | |
| | TCCCAGGTATGCTCCCACCTCCACCTGCCCCACTCACCACCTCTGCT | |
| | AGTTCCAGACACCTCCCAAGCACGCAGCAATGCAGCTCAAAACGCTT | |
| | AGCCTAGCCACACCCCCACGGGAAACAGCAGTGATTAACCTTTAGCA | |
| | ATAAACGAAAGTTTAACTAAGCTATACTAACCCCAGGGTTGGTCAATT | |
| | TCGTGCCAGCCACACCCTGGAGCTAGCAAAAAAAA | |
| Pan-CoV-Flu | CTCGACATTGATTATTGACTAGTTATTAATAGTAATCAATTACGGGGT | 406 |
| Vaccine #18 | CATTAGTTCATAGCCCATATATGGAGTTCCGCGTTACATAACTTACGG | |
| CoV-Omicron | TAAATGGCCCGCCTGGCTGACCGCCCAACGACCCCCGCCCATTGAC | |
| BA.2 Spike(2 | GTCAATAATGACGTATGTTCCCATAGTAACGCCAATAGGGACTTTCCA | |
| proline | TTGACGTCAATGGGTGGAGTATTTACGGTAAACTGCCCACTTGGCAG | |
| substitution)- | TACATCAAGTGTATCATATGCCAAGTACGCCCCCTATTGACGTCAATG | |
| Influenza (HA) | ACGGTAAATGGCCCGCCTGGCATTATGCCCAGTACATGACCTTATGG | |
| | GACTTTCCTACTTGGCAGTACATCTACGTATTAGTCATCGCTATTACC | |
| | ATGGTCGAGGTGAGCCCCACGTTCTGCTTCACTCTCCCCATCTCCCC | |
| | CCCCTCCCCACCCCCAATTTTGTATTTATTTATTTTTTAATTATTTTGT | |
| | GCAGCGATGGGGGCGGGGGGGGGGGGGGGGCGCGCGCCAGGCG | |
| | GGGCGGGGCGGGGCGAGGGGCGGGGCGGGGCGAGGCGGAGAGG | |
| | TGCGGCGGCAGCCAATCAGAGCGGCGCGCTCCGAAAGTTTCCTTTT | |
| | ATGGCGAGGCGGCGGCGGCGGCGGCCCTATAAAAAGCGAAGCGCG | |
| | CGGCGGGCGGGAGTCGCTGCGCGCTGCCTTCGCCCCGTGCCCCGC | |
| | TCCGCCGCCGCCTCGCGCCGCCCGCCCCGGCTCTGACTGACCGCG | |
| | TTACTCCCACAGGTGAGCGGGCGGGACGGCCCTTCTCCTCCGGGCT | |
| | GTAATTAGCGCTTGGTTTAATGACGGCTTGTTTCTTTTCTGTGGCTGC | |
| | GTGAAAGCCTTGAGGGGCTCCGGGAGGGCCCTTTGTGCGGGGGGA | |
| | GCGGCTCGGGGGGTGCGTGCGTGTGTGTGTGCGTGGGGAGCGCCG | |
| | CGTGCGGCTCCGCGCTGCCCGGCGGCTGTGAGCGCTGCGGGCGCG | |
| | GCGCGGGGCTTTGTGCGCTCCGCAGTGTGCGCGAGGGGAGCGCGG | |
| | CCGGGGGCGGTGCCCCGCGGTGCGGGGGGGGCTGCGAGGGGAAC | |
| | AAAGGCTGCGTGCGGGGTGTGTGCGTGGGGGGGTGAGCAGGGGGT | |
| | GTGGGCGCGTCGGTCGGGCTGCAACCCCCCCTGCACCCCCCTCCC | |
| | CGAGTTGCTGAGCACGGCCCGGCTTCGGGTGCGGGGCTCCGTACG | |
| | GGGCGTGGCGCGGGGCTCGCCGTGCCGGGCGGGGGGTGGCGGCA | |
| | GGTGGGGGTGCCGGGCGGGGCGGGGCCGCCTCGGGCCGGGGAG | |
| | GGCTCGGGGGAGGGGCGCGGCGGCCCCCGGAGCGCCGGCGGCTG | |
| | TCGAGGCGCGGCGAGCCGCAGCCATTGCCTTTTATGGTAATCGTGC | |
| | GAGAGGGCGCAGGGACTTCCTTTGTCCCAAATCTGTGCGGAGCCGA | |
| | AATCTGGGAGGCGCCGCCGCACCCCCTCTAGCGGGCGCGGGGCGA | |
| | AGCGGTGCGGCGCCGGCAGGAAGGAAATGGGCGGGGAGGGCCTTC | |
| | GTGCGTCGCCGCGCCGCCGTCCCCTTCTCCCTCTCCAGCCTCGGGG | |
| | CTGTCCGCGGGGGGACGGCTGCCTTCGGGGGGGACGGGGCAGGG | |
| | CGGGGTTCGGCTTCTGGCGTGTGACCGGCGGCTCTAGAGCCTCTGC | |
| | TAACCATGTTCATGCCTTCTTCTTTTTCCTACAGCTCCTGGGCAACGT | |
| | GCTGGTTATTGTGCTGTCTCATCATTTTGGCAAAGAATTGGAGAATAA | |
| | ACTAGTATTCTTCTGGTCCCCACAGACTCAGAGAGAACCCGCCACCA | |
| | TGTTCGTGTTCCTGGTGCTGCTGCCCCTGGTGAGCAGCCAGTGCGT | |
| | GAACCTGATCACCAGGACCCAGAGCTACACCAACAGCTTCACCAGG | |
| | GGCGTGTACTACCCCGACAAGGTGTTCAGGAGCAGCGTGCTGCACA | |
| | GCACCCAGGACCTGTTCCTGCCCTTCTTCAGCAACGTGACCTGGTTC | |
| | CACGCCATCCACGTGAGCGGCACCAACGGCACCAAGAGGTTCGACA | |
| | ACCCCGTGCTGCCCTTCAACGACGGCGTGTACTTCGCCAGCACCGA | |
| | GAAGAGCAACATCATCAGGGGCTGGATCTTCGGCACCACCCTGGAC | |
| | AGCAAGACCCAGAGCCTGCTGATCGTGAACAACGCCACCAACGTGG | |
| | TGATCAAGGTGTGCGAGTTCCAGTTCTGCAACGACCCCTTCCTGGAC | |
| | GTGTACTACCACAAGAACAACAAGAGCTGGATGGAGAGCGAGTTCA | |
| | GGGTGTACAGCAGCGCCAACAACTGCACCTTCGAGTACGTGAGCCA | |
| | GCCCTTCCTGATGGACCTGGAGGGCAAGCAGGGCAACTTCAAGAAC | |
| | CTGAGGGAGTTCGTGTTCAAGAACATCGACGGCTACTTCAAGATCTA | |
| | CAGCAAGCACACCCCCATCAACCTGGGCAGGGACCTGCCCCAGGGC | |
| | TTCAGCGCCCTGGAGCCCCTGGTGGACCTGCCCATCGGCATCAACA | |
| | TCACCAGGTTCCAGACCCTGCTGGCCCTGCACAGGAGCTACCTGAC | |
| | CCCCGGCGACAGCAGCAGCGGCTGGACCGCCGGCGCCGCCGCCTA | |
| | CTACGTGGGCTACCTGCAGCCCAGGACCTTCCTGCTGAAGTACAAC | |
| | GAGAACGGCACCATCACCGACGCCGTGGACTGCGCCCTGGACCCC | |
| | CTGAGCGAGACCAAGTGCACCCTGAAGAGCTTCACCGTGGAGAAGG | |
| | GCATCTACCAGACCAGCAACTTCAGGGTGCAGCCCACCGAGAGCAT | |
| | CGTGAGGTTCCCCAACATCACCAACCTGTGCCCCTTCGACGAGGTGT | |
| | TCAACGCCACCAGGTTCGCCAGCGTGTACGCCTGGAACAGGAAGAG | |
| | GATCAGCAACTGCGTGGCCGACTACAGCGTGCTGTACAACTTCGCC | |
| | CCCTTCTTCGCCTTCAAGTGCTACGGCGTGAGCCCCACCAAGCTGAA | |
| | CGACCTGTGCTTCACCAACGTGTACGCCGACAGCTTCGTGATCAGG | |
| | GGCAACGAGGTGAGCCAGATCGCCCCCGGCCAGACCGGCAACATC | |
| | GCCGACTACAACTACAAGCTGCCCGACGACTTCACCGGCTGCGTGA | |
| | TCGCCTGGAACAGCAACAAGCTGGACAGCAAGGTGGGCGGCAACTA | |
| | CAACTACCTGTACAGGCTGTTCAGGAAGAGCAACCTGAAGCCCTTCG | |
| | AGAGGGACATCAGCACCGAGATCTACCAGGCCGGCAACAAGCCCTG | |
| | CAACGGCGTGGCCGGCTTCAACTGCTACTTCCCCCTGAGGAGCTAC | |
| | GGCTTCAGGCCCACCTACGGCGTGGGCCACCAGCCCTACAGGGTG | |
| | GTGGTGCTGAGCTTCGAGCTGCTGCACGCCCCCGCCACCGTGTGCG | |
| | GCCCCAAGAAGAGCACCAACCTGGTGAAGAACAAGTGCGTGAACTT | |
| | CAACTTCAACGGCCTGACCGGCACCGGCGTGCTGACCGAGAGCAAC | |
| | AAGAAGTTCCTGCCCTTCCAGCAGTTCGGCAGGGACATCGCCGACA | |
| | CCACCGACGCCGTGAGGGACCCCCAGACCCTGGAGATCCTGGACAT | |
| | CACCCCCTGCAGCTTCGGCGGCGTGAGCGTGATCACCCCCGGCACC | |
| | AACACCAGCAACCAGGTGGCCGTGCTGTACCAGGGCGTGAACTGCA | |
| | CCGAGGTGCCCGTGGCCATCCACGCCGACCAGCTGACCCCCACCTG | |
| | GAGGGTGTACAGCACCGGCAGCAACGTGTTCCAGACCAGGGCCGG | |
| | CTGCCTGATCGGCGCCGAGTACGTGAACAACAGCTACGAGTGCGAC | |
| | ATCCCCATCGGCGCCGGCATCTGCGCCAGCTACCAGACCCAGACCA | |
| | AGAGCCACCAGCAGGCCCAGAGCGTGGCCAGCCAGAGCATCATCG | |
| | CCTACACCATGAGCCTGGGCGCCGAGAACAGCGTGGCCTACAGCAA | |
| | CAACAGCATCGCCATCCCCACCAACTTCACCATCAGCGTGACCACCG | |
| | AGATCCTGCCCGTGAGCATGACCAAGACCAGCGTGGACTGCACCAT | |
| | GTACATCTGCGGCGACAGCACCGAGTGCAGCAACCTGCTGCTGCAG | |
| | TACGGCAGCTTCTGCACCCAGCTGAAGAGGGCCCTGACCGGCATCG | |
| | CCGTGGAGCAGGACAAGAACACCCAGGAGGTGTTCGCCCAGGTGAA | |
| | GCAGATCTACAAGACCCCCCCCATCAAGTACTTCGGCGGCTTCAACT | |
| | TCAGCCAGATCCTGCCCGACCCCAGCAAGCCCAGCAAGAGGAGCTT | |
| | CATCGAGGACCTGCTGTTCAACAAGGTGACCCTGGCCGACGCCGGC | |
| | TTCATCAAGCAGTACGGCGACTGCCTGGGCGACATCGCCGCCAGGG | |
| | ACCTGATCTGCGCCCAGAAGTTCAACGGCCTGACCGTGCTGCCCCC | |
| | CCTGCTGACCGACGAGATGATCGCCCAGTACACCAGCGCCCTGCTG | |
| | GCCGGCACCATCACCAGCGGCTGGACCTTCGGCGCCGGCGCCGCC | |
| | CTGCAGATCCCCTTCGCCATGCAGATGGCCTACAGGTTCAACGGCAT | |
| | CGGCGTGACCCAGAACGTGCTGTACGAGAACCAGAAGCTGATCGCC | |
| | AACCAGTTCAACAGCGCCATCGGCAAGATCCAGGACAGCCTGAGCA | |
| | GCACCGCCAGCGCCCTGGGCAAGCTGCAGGACGTGGTGAACCACA | |
| | ACGCCCAGGCCCTGAACACCCTGGTGAAGCAGCTGAGCAGCAAGTT | |
| | CGGCGCCATCAGCAGCGTGCTGAACGACATCCTGAGCAGGCTGGAC | |
| | CCCCCCGAGGCCGAGGTGCAGATCGACAGGCTGATCACCGGCAGG | |
| | CTGCAGAGCCTGCAGACCTACGTGACCCAGCAGCTGATCAGGGCCG | |
| | CCGAGATCAGGGCCAGCGCCAACCTGGCCGCCACCAAGATGAGCG | |
| | AGTGCGTGCTGGGCCAGAGCAAGAGGGTGGACTTCTGCGGCAAGG | |
| | GCTACCACCTGATGAGCTTCCCCCAGAGCGCCCCCCACGGCGTGGT | |
| | GTTCCTGCACGTGACCTACGTGCCCGCCCAGGAGAAGAACTTCACC | |
| | ACCGCCCCCGCCATCTGCCACGACGGCAAGGCCCACTTCCCCAGGG | |
| | AGGGCGTGTTCGTGAGCAACGGCACCCACTGGTTCGTGACCCAGAG | |
| | GAACTTCTACGAGCCCCAGATCATCACCACCGACAACACCTTCGTGA | |
| | GCGGCAACTGCGACGTGGTGATCGGCATCGTGAACAACACCGTGTA | |
| | CGACCCCCTGCAGCCCGAGCTGGACAGCTTCAAGGAGGAGCTGGAC | |
| | AAGTACTTCAAGAACCACACCAGCCCCGACGTGGACCTGGGCGACA | |
| | TCAGCGGCATCAACGCCAGCGTGGTGAACATCCAGAAGGAGATCGA | |
| | CAGGCTGAACGAGGTGGCCAAGAACCTGAACGAGAGCCTGATCGAC | |
| | CTGCAGGAGCTGGGCAAGTACGAGCAGTACATCAAGTGGCCCTGGT | |
| | ACATCTGGCTGGGCTTCATCGCCGGCCTGATCGCCATCGTGATGGT | |
| | GACCATCATGCTGTGCTGCATGACCAGCTGCTGCAGCTGCCTGAAG | |
| | GGCTGCTGCAGCTGCGGCAGCTGCTGCAAGTTCGACGAGGACGACA | |
| | GCGAGCCCGTGCTGAAGGGCGTGAAGCTGCACTACACCGGAAGCG | |
| | GAGCCACGAACTTCTCTCTGTTAAAGCAAGCAGGAGATGTTGAAGAA | |
| | AACCCCGGGCCTATGAAGGCAATACTAGTAGTTCTGCTATATACATTT | |
| | GCAACCGCAAATGCAGACACATTATGTATAGGTTATCATGCGAACAA | |
| | CTCAACAGACACTGTAGACACAGTACTAGAAAAGAATGTAACAGTAA | |
| | CACACTCTGTTAACCTTCTAGAAGACAAGCATAACGGGAAACTATGC | |
| | AAACTAAGAGGGGTAGCCCCATTGCATTTGGGTAAATGTAACATTGC | |
| | TGGCTGGATCCTGGGAAATCCAGAGTGTGAATCACTCTCCACAGCAA | |
| | GCTCATGGTCCTACATTGTGGAAACATCTAGTTCAGACAATGGAACG | |
| | TGTTACCCAGGAGATTTCATCGATTATGAGGAGCTAAGAGAGCAATT | |
| | GAGCTCAGTGTCATCATTTGAAAGGTTTGAGATATTCCCCAAGACAA | |
| | GTTCATGGCCCAATCATGACTCGAACAAAGGTGTAACGACAGCATGT | |
| | CCTCATGCTGGAGCAAAAAGCTTCTACAAAAATTTAATATGGCTAGTT | |
| | AAAAAAGGAAATTCATACCCAAAGCTCAGCAAATCCTACATTAATGAT | |
| | AAAGGGAAAGAAGTCCTCGTGCTATGGGGCATTCACCATCCACCTAC | |
| | TAGTGCTGACCAACAAAGTCTCTATCAGAATGCAGATGCATATGTTTT | |
| | TGTGGGGACATCAAGATACAGCAAGAAGTTCAAGCCGGAAATAGCAA | |
| | TAAGGCCCAAAGTGAGGGATCAAGAAGGGAGAATGAACTATTACTGG | |
| | ACACTAGTAGAGCCGGGAGACAAAATAACATTCGAAGCAACTGGAAA | |
| | TCTAGTGGTACCGAGATATGCATTCGCAATGGAAAGAAATGCTGGAT | |
| | CTGGTATTATCATTTCAGATACACCAGTCCACGATTGCAATACAACTT | |
| | GTCAGACACCCAAGGGTGCTATAAACACCAGCCTCCCATTTCAGAAC | |
| | ATACATCCGATCACAATTGGAAAATGTCCAAAATATGTAAAAAGCACA | |
| | AAATTGAGACTGGCCACAGGATTGAGGAATGTCCCGTCCATTCAATC | |
| | TAGAGGCCTATTTGGGGCCATTGCCGGTTTCATTGAAGGGGGGTGG | |
| | ACAGGGATGGTAGATGGATGGTACGGTTATCACCATCAAAATGAGCA | |
| | GGGGTCAGGATATGCAGCCGACCTGAAGAGCACACAGAATGCCATT | |
| | GACGAGATTACTAACAAAGTAAACTCTGTTATTGAAAAAATGAATACA | |
| | CAGTTCACAGCAGTAGGTAAAGAGTTCAACCACCTGGAAAAAAGAAT | |
| | AGAGAATTTAAATAAAAAAGTTGATGATGGTTTCCTGGACATTTGGAC | |
| | TTACAATGCCGAACTGTTGGTTCTATTGGAAAATGAAAGAACTTTGGA | |
| | CTACCACGATTCAAAGGTGAAGAACTTATATGAAAAGGTAAGAAGCC | |
| | AGTTAAAAAACAATGCCAAGGAAATTGGAAACGGCTGCTTTGAATTTT | |
| | ACCACAAATGTGATAACACGTGCATGGAAAGTGTCAAAAATGGGACT | |
| | TATGACTACCCAAAATACTCAGAGGAAGCAAAATTAAACAGAGAAGAA | |
| | ATAGATGGGGTAAAGCTGGAATCAACAAGGATTTACCAGATTTTGGC | |
| | GATCTATTCAACCGTCGCCAGTTCATTGGTACTGGTAGTCTCCCTGG | |
| | GGGCAATCAGTTTCTGGATGTGCTCTAATGGGTCTCTACAGTGTAGA | |
| | ATATGTATTTAAGGAAGCGGAGCCACGAACTTCTCTCTGTTAAAGCAA | |
| | GCAGGAGATGTTGAAGAAAACCCCGGGCCTAGCAAAAGCAGGGGAT | |
| | AATTCTATTAACCATGAAGACTATCATTGCTTTGAGCTACATTCTATGT | |
| | CTGGTTTTCGCTCAAAAACTTCCTGGAAATGACAATAGCACTGCAAC | |
| | GCTGTGCCTTGGGCACCATGCAGTACCAAACGGAACGATAGTGAAAA | |
| | CAATCACGAATGACCAAATTGAAGTTACTAATGCTACTGAGCTGGTTC | |
| | AGAATTCCTCAATAGGTGAAATATGCGACAGTCCTCATCAGATCCTTG | |
| | ATGGAGAAAACTGCACACTAATAGATGCTCTATTGGGAGACCCTCAG | |
| | TGTGATGGCTTTCAAAATAAGAAATGGGACCTTTTTGTTGAACGAAGC | |
| | AAAGCCTACAGCAACTGTTACCCTTATGATGTGCCGGATTATGCCTC | |
| | CCTTAGGTCACTAGTTGCCTCATCCGGCACACTGGAGTTTAACAATG | |
| | AAAGCTTCAATTGGGCTGGAGTCACTCAAAACGGAACAAGTTCTGCT | |
| | TGCATAAGGGGATCTAATAGTAGTTTCTTTAGTAGATTAAATTGGTTG | |
| | ACCCACTTAAACTTCAAGTACCCAGCATTGAACGTGACTATGCCAAAC | |
| | AATGAACAATTTGACAAATTGTACATTTGGGGGGTTCACCACCCGGG | |
| | TACGGACAAGGACCAAATCTTCCTGTATGCTCAATCATCAGGAAGAA | |
| | TCACAGTATCTACCAAAAGAAGCCAACAAGCTGTAATCCCGAATATC | |
| | GGATCTAGACCCAGAATAAGGAATATCCCTAGCAGAATAAGCATCTA | |
| | TTGGACAATAGTAAAACCGGGAGACATACTTTTGATTAACAGCACAG | |
| | GGAATCTAATTGCTCCTAGGGGTTACTTCAAAATACGAAGTGGGAAA | |
| | AGCTCAATAATGAGATCAGATGCACCCATTGGCAAATGCAAGTCTGA | |
| | ATGCATCACTCCAAATGGAAGCATTCCCAATGACAAACCATTCCAAAA | |
| | TGTAAACAGGATCACATACGGGGCCTGTCCCAGATATGTTAAGCAAA | |
| | GCACTCTGAAATTGGCAACAGGAATGCGAAATGTACCAGAGAAACAA | |
| | ACTAGAGGCATATTTGGCGCAATAGCGGGTTTCATAGAAAATGGTTG | |
| | GGAGGGAATGGTGGATGGTTGGTACGGTTTCAGGCATCAAAATTCTG | |
| | AGGGAAGAGGACAAGCAGCAGATCTCAAAAGCACTCAAGCAGCAAT | |
| | CGATCAAATCAATGGGAAGCTGAATCGATTGATCGGGAAAACCAACG | |
| | AGAAATTCCATCAGATTGAGAAAGAATTCTCAGAAGTAGAAGGGAGA | |
| | ATTCAGGACCTTGAGAAATATGTTGAGGACACAAAAATAGATCTCTGG | |
| | TCATACAACGCAGAGCTTCTTGTTGCCCTGGAAAACCAACATACAATT | |
| | GATCTAACTGACTCAGAAATGAACAAACTGTTTGAAAAAACAAAGAAG | |
| | CAACTGAGGGAAAATGCTGAGGATATGGGCAATGGTTGTTTCAAAAT | |
| | ATACCACAAATGTGACAATGCCTGCATAGGATCAATCAGAAATGGAA | |
| | CTTATGACCACGATGTATACAGGGATGAAGCATTAAACAACCGGTTC | |
| | CAGATCAAGGGAGTTGAGCTGAAGTCAGGGTACAAAGATTGGATCCT | |
| | ATGGATTTCCTTTGCCATATCATGTTTTTTGCTTTGTGTTGCTTTGTTG | |
| | GGGTTCATCATGTGGGCCTGCCAAAAGGGCAACATTAGGTGCAACAT | |
| | TTGCATTTGAGTGCATTAATTAAAAACACCCTTGTTTCTACTGGAAGC | |
| | GGAGCCACGAACTTCTCTCTGTTAAAGCAAGCAGGAGATGTTGAAGA | |
| | AAACCCCGGGCCTATTTTCTAATATCCACAAAATGAAGGCAATAATTG | |
| | TACTACTCATGGTAGTAACATCCAATGCAGATCGAATCTGCACTGGG | |
| | ATAACATCGTCAAACTCACCACATGTCGTCAAAACTGCTACTCAAGG | |
| | GGAGGTCAACGTGACCGGTGTAATACCACTGACAACAACACCCACCA | |
| | AATCTCATTTTGCAAATCTCAAAGGAACAGAAACCAGGGGGAAACTAT | |
| | GCCCAAAATGCCTCAACTGCACAGATCTGGATGTAGCCTTGGGCAGA | |
| | CCAAAATGCACAGGGAAAATACCCTCTGCAAGGGTTTCAATACTCCA | |
| | TGAAGTCAGACCTGTTACATCTGGGTGCTTTCCTATAATGCACGATAG | |
| | AACAAAAATTAGACAGCTGCCTAACCTTCTCCGAGGATACGAACATG | |
| | TCAGGTTATCAACTCACAACGTTATCAATGCAAAAGATGCACCAGGAA | |
| | GACCCTACGAAATTGGAACCTCAGGGTCTTGCCCTAACATTACCAAT | |
| | GGAAACGGATTCTTCGCAACAATGGCTTGGGCCGTCCCAAAAAACAA | |
| | AACAGCAACAAATCCATTAACAATAGAAGTACCATACATTTGTACAGA | |
| | AGGAGAAGACCAAATTACCGTTTGGGGGTTCCACTCTGACAACGAGA | |
| | CCCAAATGGCAAAGCTCTATGGGGACTCAAAGCCCCAGAAGTTCACC | |
| | TCATCTGCCAACGGAGTGACCACACATTACGTTTCACAGATTGGTGG | |
| | CTTCCCAAATCAAACAGAAGACGGAGGACTACCACAAAGTGGCAGAA | |
| | TTGTTGTTGATTACATGGTGCAGAAATCTGGAAAAACAGGAACAATTA | |
| | CCTATCAAAGAGGTATTTTATTGCCTCAAAAAGTGTGGTGCGCAAGT | |
| | GGCAGGAGCAAGGTAATAAAAGGATCCTTGCCCTTAATTGGAGAAGC | |
| | AGATTGCCTCCATGAAAAATACGGTGGATTAAACAAAAGCAAGCCTTA | |
| | CTACACAGGGGAACATGCAAAGGCCATAGGAAATTGCCCAATATGGG | |
| | TGAAAACACCCTTGAAGCTGGCCAATGGAACCAAATATAGACCCCCT | |
| | GCAAAACTATTAAAGGAAAGAGGTTTCTTCGGAGCCATTGCTGGTTT | |
| | CTTAGAGGGAGGATGGGAAGGAATGATTGCAGGTTGGCACGGATAC | |
| | ACATCCCATGGGGCACATGGAGTAGCGGTGGCAGCTGACCTTAAGA | |
| | GCACTCAAGAGGCCATAAACAAGATAACAAAAAATCTCAACTCTTTGA | |
| | GTGAGCTGGAAGTAAAGAATCTTCAAAGACTAAGCGGTGCCATGGAT | |
| | GAACTCCACAACGAAATACTAGAACTAGATGAGAAAGTGGATGATCT | |
| | CAGAGCTGACACAATAAGCTCACAAATAGAACTCGCAGTCCTGCTTT | |
| | CCAATGAAGGAATAATAAACAGTGAAGATGAACATCTCTTGGCGCTT | |
| | GAAAGAAAGCTGAAGAAAATGCTGGGCCCCTCTGCTGTAGAGATAG | |
| | GGAATGGATGCTTTGAAACCAAACACAAGTGCAACCAGACCTGCCTC | |
| | GACAGAATAGCTGCTGGTACCTTTGATGCAGGAGAATTTTCTCTCCC | |
| | CACCTTTGATTCACTGAATATTACTGCTGCATCTTTAAATGACGACGG | |
| | ATTGGACAATCATACTATACTGCTTTACTACTCAACTGCTGCCTCCAG | |
| | TTTGGCTGTAACACTGATGATAGCTATCTTTGTTGTTTATATGGTCTC | |
| | CAGAGACAATGTTTCTTGCTCCATTTGTCTATAAGGAAAGTTAAGCCC | |
| | TGTATTTTCCTTTATTGTAGTGCTTGTTTGCTTGTTGTCATTACAAAGA | |
| | AACGTTATTGAAAAATTGATGACTCGAGCTGGTACTGCATGCACGCA | |
| | ATGCTAGCTGCCCCTTTCCCGTCCTGGGTACCCCGAGTCTCCCCCG | |
| | ACCTCGGGTCCCAGGTATGCTCCCACCTCCACCTGCCCCACTCACC | |
| | ACCTCTGCTAGTTCCAGACACCTCCCAAGCACGCAGCAATGCAGCTC | |
| | AAAACGCTTAGCCTAGCCACACCCCCACGGGAAACAGCAGTGATTAA | |
| | CCTTTAGCAATAAACGAAAGTTTAACTAAGCTATACTAACCCCAGGGT | |
| | TGGTCAATTTCGTGCCAGCCACACCCTGGAGCTAGCAAAAAAAA | |
| Pan-CoV-Flu | TAGTTATTAATAGTAATCAATTACGGGGTCATTAGTTCATAGCCCATAT | 407 |
| Vaccine #19 | ATGGAGTTCCGCGTTACATAACTTACGGTAAATGGCCCGCCTGGCTG | |
| CoV-Wuhan | ACCGCCCAACGACCCCCGCCCATTGACGTCAATAATGACGTATGTTC | |
| Spike(HexaPro)- | CCATAGTAACGCCAATAGGGACTTTCCATTGACGTCAATGGGTGGAG | |
| influenza (HA) | TATTTACGGTAAACTGCCCACTTGGCAGTACATCAAGTGTATCATATG | |
| | CCAAGTACGCCCCCTATTGACGTCAATGACGGTAAATGGCCCGCCTG | |
| | GCATTATGCCCAGTACATGACCTTATGGGACTTTCCTACTTGGCAGTA | |
| | CATCTACGTATTAGTCATCGCTATTACCATGGTGATGCGGTTTTGGCA | |
| | GTACATCAATGGGCGTGGATAGCGGTTTGACTCACGGGGATTTCCAA | |
| | GTCTCCACCCCATTGACGTCAATGGGAGTTTGTTTTGGCACCAAAAT | |
| | CAACGGGACTTTCCAAAATGTCGTAACAACTCCGCCCCATTGACGCA | |
| | AATGGGCGGTAGGCGTGTACGGTGGGAGGTCTATATAAGCAGAGCT | |
| | GGTTTAGTGAACCGTCAGATCGAGAATAAACTAGTATTCTTCTGGTCC | |
| | CCACAGACTCAGAGAGAACCCGCCACCATGTTCGTGTTCCTGGTGCT | |
| | GCTGCCCCTGGTGAGCAGCCAGTGCGTGAACCTGACCACCAGGACC | |
| | CAGCTGCCCCCCGCCTACACCAACAGCTTCACCAGGGGCGTGTACT | |
| | ACCCCGACAAGGTGTTCAGGAGCAGCGTGCTGCACAGCACCCAGGA | |
| | CCTGTTCCTGCCCTTCTTCAGCAACGTGACCTGGTTCCACGCCATCC | |
| | ACGTGAGCGGCACCAACGGCACCAAGAGGTTCGACAACCCCGTGCT | |
| | GCCCTTCAACGACGGCGTGTACTTCGCCAGCACCGAGAAGAGCAAC | |
| | ATCATCAGGGGCTGGATCTTCGGCACCACCCTGGACAGCAAGACCC | |
| | AGAGCCTGCTGATCGTGAACAACGCCACCAACGTGGTGATCAAGGT | |
| | GTGCGAGTTCCAGTTCTGCAACGACCCCTTCCTGGGCGTGTACTACC | |
| | ACAAGAACAACAAGAGCTGGATGGAGAGCGAGTTCAGGGTGTACAG | |
| | CAGCGCCAACAACTGCACCTTCGAGTACGTGAGCCAGCCCTTCCTG | |
| | ATGGACCTGGAGGGCAAGCAGGGCAACTTCAAGAACCTGAGGGAGT | |
| | TCGTGTTCAAGAACATCGACGGCTACTTCAAGATCTACAGCAAGCAC | |
| | ACCCCCATCAACCTGGTGAGGGACCTGCCCCAGGGCTTCAGCGCCC | |
| | TGGAGCCCCTGGTGGACCTGCCCATCGGCATCAACATCACCAGGTT | |
| | CCAGACCCTGCTGGCCCTGCACAGGAGCTACCTGACCCCCGGCGAC | |
| | AGCAGCAGCGGCTGGACCGCCGGCGCCGCCGCCTACTACGTGGGC | |
| | TACCTGCAGCCCAGGACCTTCCTGCTGAAGTACAACGAGAACGGCA | |
| | CCATCACCGACGCCGTGGACTGCGCCCTGGACCCCCTGAGCGAGAC | |
| | CAAGTGCACCCTGAAGAGCTTCACCGTGGAGAAGGGCATCTACCAG | |
| | ACCAGCAACTTCAGGGTGCAGCCCACCGAGAGCATCGTGAGGTTCC | |
| | CCAACATCACCAACCTGTGCCCCTTCGGCGAGGTGTTCAACGCCAC | |
| | CAGGTTCGCCAGCGTGTACGCCTGGAACAGGAAGAGGATCAGCAAC | |
| | TGCGTGGCCGACTACAGCGTGCTGTACAACAGCGCCAGCTTCAGCA | |
| | CCTTCAAGTGCTACGGCGTGAGCCCCACCAAGCTGAACGACCTGTG | |
| | CTTCACCAACGTGTACGCCGACAGCTTCGTGATCAGGGGCGACGAG | |
| | GTGAGGCAGATCGCCCCCGGCCAGACCGGCAAGATCGCCGACTACA | |
| | ACTACAAGCTGCCCGACGACTTCACCGGCTGCGTGATCGCCTGGAA | |
| | CAGCAACAACCTGGACAGCAAGGTGGGCGGCAACTACAACTACCTG | |
| | TACAGGCTGTTCAGGAAGAGCAACCTGAAGCCCTTCGAGAGGGACA | |
| | TCAGCACCGAGATCTACCAGGCCGGCAGCACCCCCTGCAACGGCGT | |
| | GGAGGGCTTCAACTGCTACTTCCCCCTGCAGAGCTACGGCTTCCAG | |
| | CCCACCAACGGCGTGGGCTACCAGCCCTACAGGGTGGTGGTGCTGA | |
| | GCTTCGAGCTGCTGCACGCCCCCGCCACCGTGTGCGGCCCCAAGAA | |
| | GAGCACCAACCTGGTGAAGAACAAGTGCGTGAACTTCAACTTCAACG | |
| | GCCTGACCGGCACCGGCGTGCTGACCGAGAGCAACAAGAAGTTCCT | |
| | GCCCTTCCAGCAGTTCGGCAGGGACATCGCCGACACCACCGACGCC | |
| | GTGAGGGACCCCCAGACCCTGGAGATCCTGGACATCACCCCCTGCA | |
| | GCTTCGGCGGCGTGAGCGTGATCACCCCCGGCACCAACACCAGCAA | |
| | CCAGGTGGCCGTGCTGTACCAGGACGTGAACTGCACCGAGGTGCCC | |
| | GTGGCCATCCACGCCGACCAGCTGACCCCCACCTGGAGGGTGTACA | |
| | GCACCGGCAGCAACGTGTTCCAGACCAGGGCCGGCTGCCTGATCG | |
| | GCGCCGAGCACGTGAACAACAGCTACGAGTGCGACATCCCCATCGG | |
| | CGCCGGCATCTGCGCCAGCTACCAGACCCAGACCAACAGCCCCGGC | |
| | AGCGCCAGCAGCGTGGCCAGCCAGAGCATCATCGCCTACACCATGA | |
| | GCCTGGGCGCCGAGAACAGCGTGGCCTACAGCAACAACAGCATCGC | |
| | CATCCCCACCAACTTCACCATCAGCGTGACCACCGAGATCCTGCCCG | |
| | TGAGCATGACCAAGACCAGCGTGGACTGCACCATGTACATCTGCGG | |
| | CGACAGCACCGAGTGCAGCAACCTGCTGCTGCAGTACGGCAGCTTC | |
| | TGCACCCAGCTGAACAGGGCCCTGACCGGCATCGCCGTGGAGCAG | |
| | GACAAGAACACCCAGGAGGTGTTCGCCCAGGTGAAGCAGATCTACA | |
| | AGACCCCCCCCATCAAGGACTTCGGCGGCTTCAACTTCAGCCAGATC | |
| | CTGCCCGACCCCAGCAAGCCCAGCAAGAGGAGCCCCATCGAGGAC | |
| | CTGCTGTTCAACAAGGTGACCCTGGCCGACGCCGGCTTCATCAAGC | |
| | AGTACGGCGACTGCCTGGGCGACATCGCCGCCAGGGACCTGATCTG | |
| | CGCCCAGAAGTTCAACGGCCTGACCGTGCTGCCCCCCCTGCTGACC | |
| | GACGAGATGATCGCCCAGTACACCAGCGCCCTGCTGGCCGGCACCA | |
| | TCACCAGCGGCTGGACCTTCGGCGCCGGCCCCGCCCTGCAGATCC | |
| | CCTTCCCCATGCAGATGGCCTACAGGTTCAACGGCATCGGCGTGAC | |
| | CCAGAACGTGCTGTACGAGAACCAGAAGCTGATCGCCAACCAGTTCA | |
| | ACAGCGCCATCGGCAAGATCCAGGACAGCCTGAGCAGCACCCCCAG | |
| | CGCCCTGGGCAAGCTGCAGGACGTGGTGAACCAGAACGCCCAGGC | |
| | CCTGAACACCCTGGTGAAGCAGCTGAGCAGCAACTTCGGCGCCATC | |
| | AGCAGCGTGCTGAACGACATCCTGAGCAGGCTGGACCCCCCCGAGG | |
| | CCGAGGTGCAGATCGACAGGCTGATCACCGGCAGGCTGCAGAGCCT | |
| | GCAGACCTACGTGACCCAGCAGCTGATCAGGGCCGCCGAGATCAGG | |
| | GCCAGCGCCAACCTGGCCGCCACCAAGATGAGCGAGTGCGTGCTG | |
| | GGCCAGAGCAAGAGGGTGGACTTCTGCGGCAAGGGCTACCACCTGA | |
| | TGAGCTTCCCCCAGAGCGCCCCCCACGGCGTGGTGTTCCTGCACGT | |
| | GACCTACGTGCCCGCCCAGGAGAAGAACTTCACCACCGCCCCCGCC | |
| | ATCTGCCACGACGGCAAGGCCCACTTCCCCAGGGAGGGCGTGTTCG | |
| | TGAGCAACGGCACCCACTGGTTCGTGACCCAGAGGAACTTCTACGA | |
| | GCCCCAGATCATCACCACCGACAACACCTTCGTGAGCGGCAACTGC | |
| | GACGTGGTGATCGGCATCGTGAACAACACCGTGTACGACCCCCTGC | |
| | AGCCCGAGCTGGACAGCTTCAAGGAGGAGCTGGACAAGTACTTCAA | |
| | GAACCACACCAGCCCCGACGTGGACCTGGGCGACATCAGCGGCATC | |
| | AACGCCAGCGTGGTGAACATCCAGAAGGAGATCGACAGGCTGAACG | |
| | AGGTGGCCAAGAACCTGAACGAGAGCCTGATCGACCTGCAGGAGCT | |
| | GGGCAAGTACGAGCAGTACATCAAGTGGCCCTGGTACATCTGGCTG | |
| | GGCTTCATCGCCGGCCTGATCGCCATCGTGATGGTGACCATCATGCT | |
| | GTGCTGCATGACCAGCTGCTGCAGCTGCCTGAAGGGCTGCTGCAGC | |
| | TGCGGCAGCTGCTGCAAGTTCGACGAGGACGACAGCGAGCCCGTG | |
| | CTGAAGGGCGTGAAGCTGCACTACACCGGAAGCGGAGCCACGAACT | |
| | TCTCTCTGTTAAAGCAAGCAGGAGATGTTGAAGAAAACCCCGGGCCT | |
| | ATGAAGGCAATACTAGTAGTTCTGCTATATACATTTGCAACCGCAAAT | |
| | GCAGACACATTATGTATAGGTTATCATGCGAACAACTCAACAGACACT | |
| | GTAGACACAGTACTAGAAAAGAATGTAACAGTAACACACTCTGTTAAC | |
| | CTTCTAGAAGACAAGCATAACGGGAAACTATGCAAACTAAGAGGGGT | |
| | AGCCCCATTGCATTTGGGTAAATGTAACATTGCTGGCTGGATCCTGG | |
| | GAAATCCAGAGTGTGAATCACTCTCCACAGCAAGCTCATGGTCCTAC | |
| | ATTGTGGAAACATCTAGTTCAGACAATGGAACGTGTTACCCAGGAGA | |
| | TTTCATCGATTATGAGGAGCTAAGAGAGCAATTGAGCTCAGTGTCAT | |
| | CATTTGAAAGGTTTGAGATATTCCCCAAGACAAGTTCATGGCCCAATC | |
| | ATGACTCGAACAAAGGTGTAACGACAGCATGTCCTCATGCTGGAGCA | |
| | AAAAGCTTCTACAAAAATTTAATATGGCTAGTTAAAAAAGGAAATTCAT | |
| | ACCCAAAGCTCAGCAAATCCTACATTAATGATAAAGGGAAAGAAGTC | |
| | CTCGTGCTATGGGGCATTCACCATCCACCTACTAGTGCTGACCAACA | |
| | AAGTCTCTATCAGAATGCAGATGCATATGTTTTTGTGGGGACATCAAG | |
| | ATACAGCAAGAAGTTCAAGCCGGAAATAGCAATAAGGCCCAAAGTGA | |
| | GGGATCAAGAAGGGAGAATGAACTATTACTGGACACTAGTAGAGCCG | |
| | GGAGACAAAATAACATTCGAAGCAACTGGAAATCTAGTGGTACCGAG | |
| | ATATGCATTCGCAATGGAAAGAAATGCTGGATCTGGTATTATCATTTC | |
| | AGATACACCAGTCCACGATTGCAATACAACTTGTCAGACACCCAAGG | |
| | GTGCTATAAACACCAGCCTCCCATTTCAGAACATACATCCGATCACAA | |
| | TTGGAAAATGTCCAAAATATGTAAAAAGCACAAAATTGAGACTGGCCA | |
| | CAGGATTGAGGAATGTCCCGTCCATTCAATCTAGAGGCCTATTTGGG | |
| | GCCATTGCCGGTTTCATTGAAGGGGGGTGGACAGGGATGGTAGATG | |
| | GATGGTACGGTTATCACCATCAAAATGAGCAGGGGTCAGGATATGCA | |
| | GCCGACCTGAAGAGCACACAGAATGCCATTGACGAGATTACTAACAA | |
| | AGTAAACTCTGTTATTGAAAAAATGAATACACAGTTCACAGCAGTAGG | |
| | TAAAGAGTTCAACCACCTGGAAAAAAGAATAGAGAATTTAAATAAAAA | |
| | AGTTGATGATGGTTTCCTGGACATTTGGACTTACAATGCCGAACTGTT | |
| | GGTTCTATTGGAAAATGAAAGAACTTTGGACTACCACGATTCAAAGGT | |
| | GAAGAACTTATATGAAAAGGTAAGAAGCCAGTTAAAAAACAATGCCAA | |
| | GGAAATTGGAAACGGCTGCTTTGAATTTTACCACAAATGTGATAACAC | |
| | GTGCATGGAAAGTGTCAAAAATGGGACTTATGACTACCCAAAATACT | |
| | CAGAGGAAGCAAAATTAAACAGAGAAGAAATAGATGGGGTAAAGCTG | |
| | GAATCAACAAGGATTTACCAGATTTTGGCGATCTATTCAACCGTCGCC | |
| | AGTTCATTGGTACTGGTAGTCTCCCTGGGGGCAATCAGTTTCTGGAT | |
| | GTGCTCTAATGGGTCTCTACAGTGTAGAATATGTATTTAAGGAAGCG | |
| | GAGCCACGAACTTCTCTCTGTTAAAGCAAGCAGGAGATGTTGAAGAA | |
| | AACCCCGGGCCTAGCAAAAGCAGGGGATAATTCTATTAACCATGAAG | |
| | ACTATCATTGCTTTGAGCTACATTCTATGTCTGGTTTTCGCTCAAAAA | |
| | CTTCCTGGAAATGACAATAGCACTGCAACGCTGTGCCTTGGGCACCA | |
| | TGCAGTACCAAACGGAACGATAGTGAAAACAATCACGAATGACCAAA | |
| | TTGAAGTTACTAATGCTACTGAGCTGGTTCAGAATTCCTCAATAGGTG | |
| | AAATATGCGACAGTCCTCATCAGATCCTTGATGGAGAAAACTGCACA | |
| | CTAATAGATGCTCTATTGGGAGACCCTCAGTGTGATGGCTTTCAAAAT | |
| | AAGAAATGGGACCTTTTTGTTGAACGAAGCAAAGCCTACAGCAACTG | |
| | TTACCCTTATGATGTGCCGGATTATGCCTCCCTTAGGTCACTAGTTGC | |
| | CTCATCCGGCACACTGGAGTTTAACAATGAAAGCTTCAATTGGGCTG | |
| | GAGTCACTCAAAACGGAACAAGTTCTGCTTGCATAAGGGGATCTAAT | |
| | AGTAGTTTCTTTAGTAGATTAAATTGGTTGACCCACTTAAACTTCAAGT | |
| | ACCCAGCATTGAACGTGACTATGCCAAACAATGAACAATTTGACAAAT | |
| | TGTACATTTGGGGGGTTCACCACCCGGGTACGGACAAGGACCAAAT | |
| | CTTCCTGTATGCTCAATCATCAGGAAGAATCACAGTATCTACCAAAAG | |
| | AAGCCAACAAGCTGTAATCCCGAATATCGGATCTAGACCCAGAATAA | |
| | GGAATATCCCTAGCAGAATAAGCATCTATTGGACAATAGTAAAACCG | |
| | GGAGACATACTTTTGATTAACAGCACAGGGAATCTAATTGCTCCTAG | |
| | GGGTTACTTCAAAATACGAAGTGGGAAAAGCTCAATAATGAGATCAG | |
| | ATGCACCCATTGGCAAATGCAAGTCTGAATGCATCACTCCAAATGGA | |
| | AGCATTCCCAATGACAAACCATTCCAAAATGTAAACAGGATCACATAC | |
| | GGGGCCTGTCCCAGATATGTTAAGCAAAGCACTCTGAAATTGGCAAC | |
| | AGGAATGCGAAATGTACCAGAGAAACAAACTAGAGGCATATTTGGCG | |
| | CAATAGCGGGTTTCATAGAAAATGGTTGGGAGGGAATGGTGGATGGT | |
| | TGGTACGGTTTCAGGCATCAAAATTCTGAGGGAAGAGGACAAGCAGC | |
| | AGATCTCAAAAGCACTCAAGCAGCAATCGATCAAATCAATGGGAAGC | |
| | TGAATCGATTGATCGGGAAAACCAACGAGAAATTCCATCAGATTGAG | |
| | AAAGAATTCTCAGAAGTAGAAGGGAGAATTCAGGACCTTGAGAAATA | |
| | TGTTGAGGACACAAAAATAGATCTCTGGTCATACAACGCAGAGCTTC | |
| | TTGTTGCCCTGGAAAACCAACATACAATTGATCTAACTGACTCAGAAA | |
| | TGAACAAACTGTTTGAAAAAACAAAGAAGCAACTGAGGGAAAATGCT | |
| | GAGGATATGGGCAATGGTTGTTTCAAAATATACCACAAATGTGACAAT | |
| | GCCTGCATAGGATCAATCAGAAATGGAACTTATGACCACGATGTATA | |
| | CAGGGATGAAGCATTAAACAACCGGTTCCAGATCAAGGGAGTTGAGC | |
| | TGAAGTCAGGGTACAAAGATTGGATCCTATGGATTTCCTTTGCCATAT | |
| | CATGTTTTTTGCTTTGTGTTGCTTTGTTGGGGTTCATCATGTGGGCCT | |
| | GCCAAAAGGGCAACATTAGGTGCAACATTTGCATTTGAGTGCATTAAT | |
| | TAAAAACACCCTTGTTTCTACTGGAAGCGGAGCCACGAACTTCTCTCT | |
| | GTTAAAGCAAGCAGGAGATGTTGAAGAAAACCCCGGGCCTATTTTCT | |
| | AATATCCACAAAATGAAGGCAATAATTGTACTACTCATGGTAGTAACA | |
| | TCCAATGCAGATCGAATCTGCACTGGGATAACATCGTCAAACTCACC | |
| | ACATGTCGTCAAAACTGCTACTCAAGGGGAGGTCAACGTGACCGGT | |
| | GTAATACCACTGACAACAACACCCACCAAATCTCATTTTGCAAATCTC | |
| | AAAGGAACAGAAACCAGGGGGAAACTATGCCCAAAATGCCTCAACTG | |
| | CACAGATCTGGATGTAGCCTTGGGCAGACCAAAATGCACAGGGAAAA | |
| | TACCCTCTGCAAGGGTTTCAATACTCCATGAAGTCAGACCTGTTACAT | |
| | CTGGGTGCTTTCCTATAATGCACGATAGAACAAAAATTAGACAGCTG | |
| | CCTAACCTTCTCCGAGGATACGAACATGTCAGGTTATCAACTCACAA | |
| | CGTTATCAATGCAAAAGATGCACCAGGAAGACCCTACGAAATTGGAA | |
| | CCTCAGGGTCTTGCCCTAACATTACCAATGGAAACGGATTCTTCGCA | |
| | ACAATGGCTTGGGCCGTCCCAAAAAACAAAACAGCAACAAATCCATT | |
| | AACAATAGAAGTACCATACATTTGTACAGAAGGAGAAGACCAAATTAC | |
| | CGTTTGGGGGTTCCACTCTGACAACGAGACCCAAATGGCAAAGCTCT | |
| | ATGGGGACTCAAAGCCCCAGAAGTTCACCTCATCTGCCAACGGAGT | |
| | GACCACACATTACGTTTCACAGATTGGTGGCTTCCCAAATCAAACAG | |
| | AAGACGGAGGACTACCACAAAGTGGCAGAATTGTTGTTGATTACATG | |
| | GTGCAGAAATCTGGAAAAACAGGAACAATTACCTATCAAAGAGGTATT | |
| | TTATTGCCTCAAAAAGTGTGGTGCGCAAGTGGCAGGAGCAAGGTAAT | |
| | AAAAGGATCCTTGCCCTTAATTGGAGAAGCAGATTGCCTCCATGAAA | |
| | AATACGGTGGATTAAACAAAAGCAAGCCTTACTACACAGGGGAACAT | |
| | GCAAAGGCCATAGGAAATTGCCCAATATGGGTGAAAACACCCTTGAA | |
| | GCTGGCCAATGGAACCAAATATAGACCCCCTGCAAAACTATTAAAGG | |
| | AAAGAGGTTTCTTCGGAGCCATTGCTGGTTTCTTAGAGGGAGGATGG | |
| | GAAGGAATGATTGCAGGTTGGCACGGATACACATCCCATGGGGCAC | |
| | ATGGAGTAGCGGTGGCAGCTGACCTTAAGAGCACTCAAGAGGCCAT | |
| | AAACAAGATAACAAAAAATCTCAACTCTTTGAGTGAGCTGGAAGTAAA | |
| | GAATCTTCAAAGACTAAGCGGTGCCATGGATGAACTCCACAACGAAA | |
| | TACTAGAACTAGATGAGAAAGTGGATGATCTCAGAGCTGACACAATA | |
| | AGCTCACAAATAGAACTCGCAGTCCTGCTTTCCAATGAAGGAATAATA | |
| | AACAGTGAAGATGAACATCTCTTGGCGCTTGAAAGAAAGCTGAAGAA | |
| | AATGCTGGGCCCCTCTGCTGTAGAGATAGGGAATGGATGCTTTGAAA | |
| | CCAAACACAAGTGCAACCAGACCTGCCTCGACAGAATAGCTGCTGGT | |
| | ACCTTTGATGCAGGAGAATTTTCTCTCCCCACCTTTGATTCACTGAAT | |
| | ATTACTGCTGCATCTTTAAATGACGACGGATTGGACAATCATACTATA | |
| | CTGCTTTACTACTCAACTGCTGCCTCCAGTTTGGCTGTAACACTGATG | |
| | ATAGCTATCTTTGTTGTTTATATGGTCTCCAGAGACAATGTTTCTTGCT | |
| | CCATTTGTCTATAAGGAAAGTTAAGCCCTGTATTTTCCTTTATTGTAGT | |
| | GCTTGTTTGCTTGTTGTCATTACAAAGAAACGTTATTGAAAAATTGAT | |
| | GACTCGAGCTGGTACTGCATGCACGCAATGCTAGCTGCCCCTTTCCC | |
| | GTCCTGGGTACCCCGAGTCTCCCCCGACCTCGGGTCCCAGGTATGC | |
| | TCCCACCTCCACCTGCCCCACTCACCACCTCTGCTAGTTCCAGACAC | |
| | CTCCCAAGCACGCAGCAATGCAGCTCAAAACGCTTAGCCTAGCCACA | |
| | CCCCCACGGGAAACAGCAGTGATTAACCTTTAGCAATAAACGAAAGT | |
| | TTAACTAAGCTATACTAACCCCAGGGTTGGTCAATTTCGTGCCAGCC | |
| | ACACCCTGGAGCTAGCAAAAAAAA | |
| Pan-CoV-Flu | TAGTTATTAATAGTAATCAATTACGGGGTCATTAGTTCATAGCCCATAT | 408 |
| Vaccine #20 | ATGGAGTTCCGCGTTACATAACTTACGGTAAATGGCCCGCCTGGCTG | |
| CoV-Wuhan | ACCGCCCAACGACCCCCGCCCATTGACGTCAATAATGACGTATGTTC | |
| Spike(2 proline | CCATAGTAACGCCAATAGGGACTTTCCATTGACGTCAATGGGTGGAG | |
| substitution)- | TATTTACGGTAAACTGCCCACTTGGCAGTACATCAAGTGTATCATATG | |
| Influenza (HA) | CCAAGTACGCCCCCTATTGACGTCAATGACGGTAAATGGCCCGCCTG | |
| | GCATTATGCCCAGTACATGACCTTATGGGACTTTCCTACTTGGCAGTA | |
| | CATCTACGTATTAGTCATCGCTATTACCATGGTGATGCGGTTTTGGCA | |
| | GTACATCAATGGGCGTGGATAGCGGTTTGACTCACGGGGATTTCCAA | |
| | GTCTCCACCCCATTGACGTCAATGGGAGTTTGTTTTGGCACCAAAAT | |
| | CAACGGGACTTTCCAAAATGTCGTAACAACTCCGCCCCATTGACGCA | |
| | AATGGGCGGTAGGCGTGTACGGTGGGAGGTCTATATAAGCAGAGCT | |
| | GGTTTAGTGAACCGTCAGATCGAGAATAAACTAGTATTCTTCTGGTCC | |
| | CCACAGACTCAGAGAGAACCCGCCACCATGTTCGTGTTCCTGGTGCT | |
| | GCTGCCCCTGGTGAGCAGCCAGTGCGTGAACCTGACCACCAGGACC | |
| | CAGCTGCCCCCCGCCTACACCAACAGCTTCACCAGGGGCGTGTACT | |
| | ACCCCGACAAGGTGTTCAGGAGCAGCGTGCTGCACAGCACCCAGGA | |
| | CCTGTTCCTGCCCTTCTTCAGCAACGTGACCTGGTTCCACGCCATCC | |
| | ACGTGAGCGGCACCAACGGCACCAAGAGGTTCGACAACCCCGTGCT | |
| | GCCCTTCAACGACGGCGTGTACTTCGCCAGCACCGAGAAGAGCAAC | |
| | ATCATCAGGGGCTGGATCTTCGGCACCACCCTGGACAGCAAGACCC | |
| | AGAGCCTGCTGATCGTGAACAACGCCACCAACGTGGTGATCAAGGT | |
| | GTGCGAGTTCCAGTTCTGCAACGACCCCTTCCTGGGCGTGTACTACC | |
| | ACAAGAACAACAAGAGCTGGATGGAGAGCGAGTTCAGGGTGTACAG | |
| | CAGCGCCAACAACTGCACCTTCGAGTACGTGAGCCAGCCCTTCCTG | |
| | ATGGACCTGGAGGGCAAGCAGGGCAACTTCAAGAACCTGAGGGAGT | |
| | TCGTGTTCAAGAACATCGACGGCTACTTCAAGATCTACAGCAAGCAC | |
| | ACCCCCATCAACCTGGTGAGGGACCTGCCCCAGGGCTTCAGCGCCC | |
| | TGGAGCCCCTGGTGGACCTGCCCATCGGCATCAACATCACCAGGTT | |
| | CCAGACCCTGCTGGCCCTGCACAGGAGCTACCTGACCCCCGGCGAC | |
| | AGCAGCAGCGGCTGGACCGCCGGCGCCGCCGCCTACTACGTGGGC | |
| | TACCTGCAGCCCAGGACCTTCCTGCTGAAGTACAACGAGAACGGCA | |
| | CCATCACCGACGCCGTGGACTGCGCCCTGGACCCCCTGAGCGAGAC | |
| | CAAGTGCACCCTGAAGAGCTTCACCGTGGAGAAGGGCATCTACCAG | |
| | ACCAGCAACTTCAGGGTGCAGCCCACCGAGAGCATCGTGAGGTTCC | |
| | CCAACATCACCAACCTGTGCCCCTTCGGCGAGGTGTTCAACGCCAC | |
| | CAGGTTCGCCAGCGTGTACGCCTGGAACAGGAAGAGGATCAGCAAC | |
| | TGCGTGGCCGACTACAGCGTGCTGTACAACAGCGCCAGCTTCAGCA | |
| | CCTTCAAGTGCTACGGCGTGAGCCCCACCAAGCTGAACGACCTGTG | |
| | CTTCACCAACGTGTACGCCGACAGCTTCGTGATCAGGGGCGACGAG | |
| | GTGAGGCAGATCGCCCCCGGCCAGACCGGCAAGATCGCCGACTACA | |
| | ACTACAAGCTGCCCGACGACTTCACCGGCTGCGTGATCGCCTGGAA | |
| | CAGCAACAACCTGGACAGCAAGGTGGGCGGCAACTACAACTACCTG | |
| | TACAGGCTGTTCAGGAAGAGCAACCTGAAGCCCTTCGAGAGGGACA | |
| | TCAGCACCGAGATCTACCAGGCCGGCAGCACCCCCTGCAACGGCGT | |
| | GGAGGGCTTCAACTGCTACTTCCCCCTGCAGAGCTACGGCTTCCAG | |
| | CCCACCAACGGCGTGGGCTACCAGCCCTACAGGGTGGTGGTGCTGA | |
| | GCTTCGAGCTGCTGCACGCCCCCGCCACCGTGTGCGGCCCCAAGAA | |
| | GAGCACCAACCTGGTGAAGAACAAGTGCGTGAACTTCAACTTCAACG | |
| | GCCTGACCGGCACCGGCGTGCTGACCGAGAGCAACAAGAAGTTCCT | |
| | GCCCTTCCAGCAGTTCGGCAGGGACATCGCCGACACCACCGACGCC | |
| | GTGAGGGACCCCCAGACCCTGGAGATCCTGGACATCACCCCCTGCA | |
| | GCTTCGGCGGCGTGAGCGTGATCACCCCCGGCACCAACACCAGCAA | |
| | CCAGGTGGCCGTGCTGTACCAGGACGTGAACTGCACCGAGGTGCCC | |
| | GTGGCCATCCACGCCGACCAGCTGACCCCCACCTGGAGGGTGTACA | |
| | GCACCGGCAGCAACGTGTTCCAGACCAGGGCCGGCTGCCTGATCG | |
| | GCGCCGAGCACGTGAACAACAGCTACGAGTGCGACATCCCCATCGG | |
| | CGCCGGCATCTGCGCCAGCTACCAGACCCAGACCAACAGCCCCAGG | |
| | AGGGCCAGGAGCGTGGCCAGCCAGAGCATCATCGCCTACACCATGA | |
| | GCCTGGGCGCCGAGAACAGCGTGGCCTACAGCAACAACAGCATCGC | |
| | CATCCCCACCAACTTCACCATCAGCGTGACCACCGAGATCCTGCCCG | |
| | TGAGCATGACCAAGACCAGCGTGGACTGCACCATGTACATCTGCGG | |
| | CGACAGCACCGAGTGCAGCAACCTGCTGCTGCAGTACGGCAGCTTC | |
| | TGCACCCAGCTGAACAGGGCCCTGACCGGCATCGCCGTGGAGCAG | |
| | GACAAGAACACCCAGGAGGTGTTCGCCCAGGTGAAGCAGATCTACA | |
| | AGACCCCCCCCATCAAGGACTTCGGCGGCTTCAACTTCAGCCAGATC | |
| | CTGCCCGACCCCAGCAAGCCCAGCAAGAGGAGCTTCATCGAGGACC | |
| | TGCTGTTCAACAAGGTGACCCTGGCCGACGCCGGCTTCATCAAGCA | |
| | GTACGGCGACTGCCTGGGCGACATCGCCGCCAGGGACCTGATCTGC | |
| | GCCCAGAAGTTCAACGGCCTGACCGTGCTGCCCCCCCTGCTGACCG | |
| | ACGAGATGATCGCCCAGTACACCAGCGCCCTGCTGGCCGGCACCAT | |
| | CACCAGCGGCTGGACCTTCGGCGCCGGCGCCGCCCTGCAGATCCC | |
| | CTTCGCCATGCAGATGGCCTACAGGTTCAACGGCATCGGCGTGACC | |
| | CAGAACGTGCTGTACGAGAACCAGAAGCTGATCGCCAACCAGTTCAA | |
| | CAGCGCCATCGGCAAGATCCAGGACAGCCTGAGCAGCACCGCCAGC | |
| | GCCCTGGGCAAGCTGCAGGACGTGGTGAACCAGAACGCCCAGGCC | |
| | CTGAACACCCTGGTGAAGCAGCTGAGCAGCAACTTCGGCGCCATCA | |
| | GCAGCGTGCTGAACGACATCCTGAGCAGGCTGGACCCCCCCGAGG | |
| | CCGAGGTGCAGATCGACAGGCTGATCACCGGCAGGCTGCAGAGCCT | |
| | GCAGACCTACGTGACCCAGCAGCTGATCAGGGCCGCCGAGATCAGG | |
| | GCCAGCGCCAACCTGGCCGCCACCAAGATGAGCGAGTGCGTGCTG | |
| | GGCCAGAGCAAGAGGGTGGACTTCTGCGGCAAGGGCTACCACCTGA | |
| | TGAGCTTCCCCCAGAGCGCCCCCCACGGCGTGGTGTTCCTGCACGT | |
| | GACCTACGTGCCCGCCCAGGAGAAGAACTTCACCACCGCCCCCGCC | |
| | ATCTGCCACGACGGCAAGGCCCACTTCCCCAGGGAGGGCGTGTTCG | |
| | TGAGCAACGGCACCCACTGGTTCGTGACCCAGAGGAACTTCTACGA | |
| | GCCCCAGATCATCACCACCGACAACACCTTCGTGAGCGGCAACTGC | |
| | GACGTGGTGATCGGCATCGTGAACAACACCGTGTACGACCCCCTGC | |
| | AGCCCGAGCTGGACAGCTTCAAGGAGGAGCTGGACAAGTACTTCAA | |
| | GAACCACACCAGCCCCGACGTGGACCTGGGCGACATCAGCGGCATC | |
| | AACGCCAGCGTGGTGAACATCCAGAAGGAGATCGACAGGCTGAACG | |
| | AGGTGGCCAAGAACCTGAACGAGAGCCTGATCGACCTGCAGGAGCT | |
| | GGGCAAGTACGAGCAGTACATCAAGTGGCCCTGGTACATCTGGCTG | |
| | GGCTTCATCGCCGGCCTGATCGCCATCGTGATGGTGACCATCATGCT | |
| | GTGCTGCATGACCAGCTGCTGCAGCTGCCTGAAGGGCTGCTGCAGC | |
| | TGCGGCAGCTGCTGCAAGTTCGACGAGGACGACAGCGAGCCCGTG | |
| | CTGAAGGGCGTGAAGCTGCACTACACCGGAAGCGGAGCCACGAACT | |
| | TCTCTCTGTTAAAGCAAGCAGGAGATGTTGAAGAAAACCCCGGGCCT | |
| | ATGAAGGCAATACTAGTAGTTCTGCTATATACATTTGCAACCGCAAAT | |
| | GCAGACACATTATGTATAGGTTATCATGCGAACAACTCAACAGACACT | |
| | GTAGACACAGTACTAGAAAAGAATGTAACAGTAACACACTCTGTTAAC | |
| | CTTCTAGAAGACAAGCATAACGGGAAACTATGCAAACTAAGAGGGGT | |
| | AGCCCCATTGCATTTGGGTAAATGTAACATTGCTGGCTGGATCCTGG | |
| | GAAATCCAGAGTGTGAATCACTCTCCACAGCAAGCTCATGGTCCTAC | |
| | ATTGTGGAAACATCTAGTTCAGACAATGGAACGTGTTACCCAGGAGA | |
| | TTTCATCGATTATGAGGAGCTAAGAGAGCAATTGAGCTCAGTGTCAT | |
| | CATTTGAAAGGTTTGAGATATTCCCCAAGACAAGTTCATGGCCCAATC | |
| | ATGACTCGAACAAAGGTGTAACGACAGCATGTCCTCATGCTGGAGCA | |
| | AAAAGCTTCTACAAAAATTTAATATGGCTAGTTAAAAAAGGAAATTCAT | |
| | ACCCAAAGCTCAGCAAATCCTACATTAATGATAAAGGGAAAGAAGTC | |
| | CTCGTGCTATGGGGCATTCACCATCCACCTACTAGTGCTGACCAACA | |
| | AAGTCTCTATCAGAATGCAGATGCATATGTTTTTGTGGGGACATCAAG | |
| | ATACAGCAAGAAGTTCAAGCCGGAAATAGCAATAAGGCCCAAAGTGA | |
| | GGGATCAAGAAGGGAGAATGAACTATTACTGGACACTAGTAGAGCCG | |
| | GGAGACAAAATAACATTCGAAGCAACTGGAAATCTAGTGGTACCGAG | |
| | ATATGCATTCGCAATGGAAAGAAATGCTGGATCTGGTATTATCATTTC | |
| | AGATACACCAGTCCACGATTGCAATACAACTTGTCAGACACCCAAGG | |
| | GTGCTATAAACACCAGCCTCCCATTTCAGAACATACATCCGATCACAA | |
| | TTGGAAAATGTCCAAAATATGTAAAAAGCACAAAATTGAGACTGGCCA | |
| | CAGGATTGAGGAATGTCCCGTCCATTCAATCTAGAGGCCTATTTGGG | |
| | GCCATTGCCGGTTTCATTGAAGGGGGGTGGACAGGGATGGTAGATG | |
| | GATGGTACGGTTATCACCATCAAAATGAGCAGGGGTCAGGATATGCA | |
| | GCCGACCTGAAGAGCACACAGAATGCCATTGACGAGATTACTAACAA | |
| | AGTAAACTCTGTTATTGAAAAAATGAATACACAGTTCACAGCAGTAGG | |
| | TAAAGAGTTCAACCACCTGGAAAAAAGAATAGAGAATTTAAATAAAAA | |
| | AGTTGATGATGGTTTCCTGGACATTTGGACTTACAATGCCGAACTGTT | |
| | GGTTCTATTGGAAAATGAAAGAACTTTGGACTACCACGATTCAAAGGT | |
| | GAAGAACTTATATGAAAAGGTAAGAAGCCAGTTAAAAAACAATGCCAA | |
| | GGAAATTGGAAACGGCTGCTTTGAATTTTACCACAAATGTGATAACAC | |
| | GTGCATGGAAAGTGTCAAAAATGGGACTTATGACTACCCAAAATACT | |
| | CAGAGGAAGCAAAATTAAACAGAGAAGAAATAGATGGGGTAAAGCTG | |
| | GAATCAACAAGGATTTACCAGATTTTGGCGATCTATTCAACCGTCGCC | |
| | AGTTCATTGGTACTGGTAGTCTCCCTGGGGGCAATCAGTTTCTGGAT | |
| | GTGCTCTAATGGGTCTCTACAGTGTAGAATATGTATTTAAGGAAGCG | |
| | GAGCCACGAACTTCTCTCTGTTAAAGCAAGCAGGAGATGTTGAAGAA | |
| | AACCCCGGGCCTAGCAAAAGCAGGGGATAATTCTATTAACCATGAAG | |
| | ACTATCATTGCTTTGAGCTACATTCTATGTCTGGTTTTCGCTCAAAAA | |
| | CTTCCTGGAAATGACAATAGCACTGCAACGCTGTGCCTTGGGCACCA | |
| | TGCAGTACCAAACGGAACGATAGTGAAAACAATCACGAATGACCAAA | |
| | TTGAAGTTACTAATGCTACTGAGCTGGTTCAGAATTCCTCAATAGGTG | |
| | AAATATGCGACAGTCCTCATCAGATCCTTGATGGAGAAAACTGCACA | |
| | CTAATAGATGCTCTATTGGGAGACCCTCAGTGTGATGGCTTTCAAAAT | |
| | AAGAAATGGGACCTTTTTGTTGAACGAAGCAAAGCCTACAGCAACTG | |
| | TTACCCTTATGATGTGCCGGATTATGCCTCCCTTAGGTCACTAGTTGC | |
| | CTCATCCGGCACACTGGAGTTTAACAATGAAAGCTTCAATTGGGCTG | |
| | GAGTCACTCAAAACGGAACAAGTTCTGCTTGCATAAGGGGATCTAAT | |
| | AGTAGTTTCTTTAGTAGATTAAATTGGTTGACCCACTTAAACTTCAAGT | |
| | ACCCAGCATTGAAGGTGACTATGCCAAACAATGAACAATTTGACAAAT | |
| | TGTACATTTGGGGGGTTCACCACCCGGGTACGGACAAGGACCAAAT | |
| | CTTCCTGTATGCTCAATCATCAGGAAGAATCACAGTATCTACCAAAAG | |
| | AAGCCAACAAGCTGTAATCCCGAATATCGGATCTAGACCCAGAATAA | |
| | GGAATATCCCTAGCAGAATAAGCATCTATTGGACAATAGTAAAACCG | |
| | GGAGACATACTTTTGATTAACAGCACAGGGAATCTAATTGCTCCTAG | |
| | GGGTTACTTCAAAATACGAAGTGGGAAAAGCTCAATAATGAGATCAG | |
| | ATGCACCCATTGGCAAATGCAAGTCTGAATGCATCACTCCAAATGGA | |
| | AGCATTCCCAATGACAAACCATTCCAAAATGTAAACAGGATCACATAC | |
| | GGGGCCTGTCCCAGATATGTTAAGCAAAGCACTCTGAAATTGGCAAC | |
| | AGGAATGCGAAATGTACCAGAGAAACAAACTAGAGGCATATTTGGCG | |
| | CAATAGCGGGTTTCATAGAAAATGGTTGGGAGGGAATGGTGGATGGT | |
| | TGGTACGGTTTCAGGCATCAAAATTCTGAGGGAAGAGGACAAGCAGC | |
| | AGATCTCAAAAGCACTCAAGCAGCAATCGATCAAATCAATGGGAAGC | |
| | TGAATCGATTGATCGGGAAAACCAACGAGAAATTCCATCAGATTGAG | |
| | AAAGAATTCTCAGAAGTAGAAGGGAGAATTCAGGACCTTGAGAAATA | |
| | TGTTGAGGACACAAAAATAGATCTCTGGTCATACAACGCAGAGCTTC | |
| | TTGTTGCCCTGGAAAACCAACATACAATTGATCTAACTGACTCAGAAA | |
| | TGAACAAACTGTTTGAAAAAACAAAGAAGCAACTGAGGGAAAATGCT | |
| | GAGGATATGGGCAATGGTTGTTTCAAAATATACCACAAATGTGACAAT | |
| | GCCTGCATAGGATCAATCAGAAATGGAACTTATGACCACGATGTATA | |
| | CAGGGATGAAGCATTAAACAACCGGTTCCAGATCAAGGGAGTTGAGC | |
| | TGAAGTCAGGGTACAAAGATTGGATCCTATGGATTTCCTTTGCCATAT | |
| | CATGTTTTTTGCTTTGTGTTGCTTTGTTGGGGTTCATCATGTGGGCCT | |
| | GCCAAAAGGGCAACATTAGGTGCAACATTTGCATTTGAGTGCATTAAT | |
| | TAAAAACACCCTTGTTTCTACTGGAAGCGGAGCCACGAACTTCTCTCT | |
| | GTTAAAGCAAGCAGGAGATGTTGAAGAAAACCCCGGGCCTATTTTCT | |
| | AATATCCACAAAATGAAGGCAATAATTGTACTACTCATGGTAGTAACA | |
| | TCCAATGCAGATCGAATCTGCACTGGGATAACATCGTCAAACTCACC | |
| | ACATGTCGTCAAAACTGCTACTCAAGGGGAGGTCAACGTGACCGGT | |
| | GTAATACCACTGACAACAACACCCACCAAATCTCATTTTGCAAATCTC | |
| | AAAGGAACAGAAACCAGGGGGAAACTATGCCCAAAATGCCTCAACTG | |
| | CACAGATCTGGATGTAGCCTTGGGCAGACCAAAATGCACAGGGAAAA | |
| | TACCCTCTGCAAGGGTTTCAATACTCCATGAAGTCAGACCTGTTACAT | |
| | CTGGGTGCTTTCCTATAATGCACGATAGAACAAAAATTAGACAGCTG | |
| | CCTAACCTTCTCCGAGGATACGAACATGTCAGGTTATCAACTCACAA | |
| | CGTTATCAATGCAAAAGATGCACCAGGAAGACCCTACGAAATTGGAA | |
| | CCTCAGGGTCTTGCCCTAACATTACCAATGGAAACGGATTCTTCGCA | |
| | ACAATGGCTTGGGCCGTCCCAAAAAACAAAACAGCAACAAATCCATT | |
| | AACAATAGAAGTACCATACATTTGTACAGAAGGAGAAGACCAAATTAC | |
| | CGTTTGGGGGTTCCACTCTGACAACGAGACCCAAATGGCAAAGCTCT | |
| | ATGGGGACTCAAAGCCCCAGAAGTTCACCTCATCTGCCAACGGAGT | |
| | GACCACACATTACGTTTCACAGATTGGTGGCTTCCCAAATCAAACAG | |
| | AAGACGGAGGACTACCACAAAGTGGCAGAATTGTTGTTGATTACATG | |
| | GTGCAGAAATCTGGAAAAACAGGAACAATTACCTATCAAAGAGGTATT | |
| | TTATTGCCTCAAAAAGTGTGGTGCGCAAGTGGCAGGAGCAAGGTAAT | |
| | AAAAGGATCCTTGCCCTTAATTGGAGAAGCAGATTGCCTCCATGAAA | |
| | AATACGGTGGATTAAACAAAAGCAAGCCTTACTACACAGGGGAACAT | |
| | GCAAAGGCCATAGGAAATTGCCCAATATGGGTGAAAACACCCTTGAA | |
| | GCTGGCCAATGGAACCAAATATAGACCCCCTGCAAAACTATTAAAGG | |
| | AAAGAGGTTTCTTCGGAGCCATTGCTGGTTTCTTAGAGGGAGGATGG | |
| | GAAGGAATGATTGCAGGTTGGCACGGATACACATCCCATGGGGCAC | |
| | ATGGAGTAGCGGTGGCAGCTGACCTTAAGAGCACTCAAGAGGCCAT | |
| | AAACAAGATAACAAAAAATCTCAACTCTTTGAGTGAGCTGGAAGTAAA | |
| | GAATCTTCAAAGACTAAGCGGTGCCATGGATGAACTCCACAACGAAA | |
| | TACTAGAACTAGATGAGAAAGTGGATGATCTCAGAGCTGACACAATA | |
| | AGCTCACAAATAGAACTCGCAGTCCTGCTTTCCAATGAAGGAATAATA | |
| | AACAGTGAAGATGAACATCTCTTGGCGCTTGAAAGAAAGCTGAAGAA | |
| | AATGCTGGGCCCCTCTGCTGTAGAGATAGGGAATGGATGCTTTGAAA | |
| | CCAAACACAAGTGCAACCAGACCTGCCTCGACAGAATAGCTGCTGGT | |
| | ACCTTTGATGCAGGAGAATTTTCTCTCCCCACCTTTGATTCACTGAAT | |
| | ATTACTGCTGCATCTTTAAATGACGACGGATTGGACAATCATACTATA | |
| | CTGCTTTACTACTCAACTGCTGCCTCCAGTTTGGCTGTAACACTGATG | |
| | ATAGCTATCTTTGTTGTTTATATGGTCTCCAGAGACAATGTTTCTTGCT | |
| | CCATTTGTCTATAAGGAAAGTTAAGCCCTGTATTTTCCTTTATTGTAGT | |
| | GCTTGTTTGCTTGTTGTCATTACAAAGAAACGTTATTGAAAAATTGAT | |
| | GACTCGAGCTGGTACTGCATGCACGCAATGCTAGCTGCCCCTTTCCC | |
| | GTCCTGGGTACCCCGAGTCTCCCCCGACCTCGGGTCCCAGGTATGC | |
| | TCCCACCTCCACCTGCCCCACTCACCACCTCTGCTAGTTCCAGACAC | |
| | CTCCCAAGCACGCAGCAATGCAGCTCAAAACGCTTAGCCTAGCCACA | |
| | CCCCCACGGGAAACAGCAGTGATTAACCTTTAGCAATAAACGAAAGT | |
| | TTAACTAAGCTATACTAACCCCAGGGTTGGTCAATTTCGTGCCAGCC | |
| | ACACCCTGGAGCTAGCAAAAAAAA | |
| Pan-CoV-Flu | CTCGACATTGATTATTGACTAGTTATTAATAGTAATCAATTACGGGGT | 409 |
| Vaccine #21 | CATTAGTTCATAGCCCATATATGGAGTTCCGCGTTACATAACTTACGG | |
| CoV-Wuhan | TAAATGGCCCGCCTGGCTGACCGCCCAACGACCCCCGCCCATTGAC | |
| Spike(HexaPro)- | GTCAATAATGACGTATGTTCCCATAGTAACGCCAATAGGGACTTTCCA | |
| Influenza (HA) | TTGACGTCAATGGGTGGAGTATTTACGGTAAACTGCCCACTTGGCAG | |
| | TACATCAAGTGTATCATATGCCAAGTACGCCCCCTATTGACGTCAATG | |
| | ACGGTAAATGGCCCGCCTGGCATTATGCCCAGTACATGACCTTATGG | |
| | GACTTTCCTACTTGGCAGTACATCTACGTATTAGTCATCGCTATTACC | |
| | ATGGTCGAGGTGAGCCCCACGTTCTGCTTCACTCTCCCCATCTCCCC | |
| | CCCCTCCCCACCCCCAATTTTGTATTTATTTATTTTTTAATTATTTTGT | |
| | GCAGCGATGGGGGCGGGGGGGGGGGGGGGGCGCGCGCCAGGCG | |
| | GGGCGGGGCGGGGCGAGGGGCGGGGCGGGGCGAGGCGGAGAGG | |
| | TGCGGCGGCAGCCAATCAGAGCGGCGCGCTCCGAAAGTTTCCTTTT | |
| | ATGGCGAGGCGGCGGCGGCGGCGGCCCTATAAAAAGCGAAGCGCG | |
| | CGGCGGGCGGGAGTCGCTGCGCGCTGCCTTCGCCCCGTGCCCCGC | |
| | TCCGCCGCCGCCTCGCGCCGCCCGCCCCGGCTCTGACTGACCGCG | |
| | TTACTCCCACAGGTGAGCGGGCGGGACGGCCCTTCTCCTCCGGGCT | |
| | GTAATTAGCGCTTGGTTTAATGACGGCTTGTTTCTTTTCTGTGGCTGC | |
| | GTGAAAGCCTTGAGGGGCTCCGGGAGGGCCCTTTGTGCGGGGGGA | |
| | GCGGCTCGGGGGGTGCGTGCGTGTGTGTGTGCGTGGGGAGCGCCG | |
| | CGTGCGGCTCCGCGCTGCCCGGCGGCTGTGAGCGCTGCGGGCGCG | |
| | GCGCGGGGCTTTGTGCGCTCCGCAGTGTGCGCGAGGGGAGCGCGG | |
| | CCGGGGGCGGTGCCCCGCGGTGCGGGGGGGGCTGCGAGGGGAAC | |
| | AAAGGCTGCGTGCGGGGTGTGTGCGTGGGGGGGTGAGCAGGGGGT | |
| | GTGGGCGCGTCGGTCGGGCTGCAACCCCCCCTGCACCCCCCTCCC | |
| | CGAGTTGCTGAGCACGGCCCGGCTTCGGGTGCGGGGCTCCGTACG | |
| | GGGCGTGGCGCGGGGCTCGCCGTGCCGGGCGGGGGGTGGCGGCA | |
| | GGTGGGGGTGCCGGGCGGGGCGGGGCCGCCTCGGGCCGGGGAG | |
| | GGCTCGGGGGAGGGGCGCGGCGGCCCCCGGAGCGCCGGCGGCTG | |
| | TCGAGGCGCGGCGAGCCGCAGCCATTGCCTTTTATGGTAATCGTGC | |
| | GAGAGGGCGCAGGGACTTCCTTTGTCCCAAATCTGTGCGGAGCCGA | |
| | AATCTGGGAGGCGCCGCCGCACCCCCTCTAGCGGGCGCGGGGCGA | |
| | AGCGGTGCGGCGCCGGCAGGAAGGAAATGGGCGGGGAGGGCCTTC | |
| | GTGCGTCGCCGCGCCGCCGTCCCCTTCTCCCTCTCCAGCCTCGGGG | |
| | CTGTCCGCGGGGGGACGGCTGCCTTCGGGGGGGACGGGGCAGGG | |
| | CGGGGTTCGGCTTCTGGCGTGTGACCGGCGGCTCTAGAGCCTCTGC | |
| | TAACCATGTTCATGCCTTCTTCTTTTTCCTACAGCTCCTGGGCAACGT | |
| | GCTGGTTATTGTGCTGTCTCATCATTTTGGCAAAGAATTGGAGAATAA | |
| | ACTAGTATTCTTCTGGTCCCCACAGACTCAGAGAGAACCCGCCACCA | |
| | TGTTCGTGTTCCTGGTGCTGCTGCCCCTGGTGAGCAGCCAGTGCGT | |
| | GAACCTGACCACCAGGACCCAGCTGCCCCCCGCCTACACCAACAGC | |
| | TTCACCAGGGGCGTGTACTACCCCGACAAGGTGTTCAGGAGCAGCG | |
| | TGCTGCACAGCACCCAGGACCTGTTCCTGCCCTTCTTCAGCAACGTG | |
| | ACCTGGTTCCACGCCATCCACGTGAGCGGCACCAACGGCACCAAGA | |
| | GGTTCGACAACCCCGTGCTGCCCTTCAACGACGGCGTGTACTTCGC | |
| | CAGCACCGAGAAGAGCAACATCATCAGGGGCTGGATCTTCGGCACC | |
| | ACCCTGGACAGCAAGACCCAGAGCCTGCTGATCGTGAACAACGCCA | |
| | CCAACGTGGTGATCAAGGTGTGCGAGTTCCAGTTCTGCAACGACCC | |
| | CTTCCTGGGCGTGTACTACCACAAGAACAACAAGAGCTGGATGGAGA | |
| | GCGAGTTCAGGGTGTACAGCAGCGCCAACAACTGCACCTTCGAGTA | |
| | CGTGAGCCAGCCCTTCCTGATGGACCTGGAGGGCAAGCAGGGCAAC | |
| | TTCAAGAACCTGAGGGAGTTCGTGTTCAAGAACATCGACGGCTACTT | |
| | CAAGATCTACAGCAAGCACACCCCCATCAACCTGGTGAGGGACCTG | |
| | CCCCAGGGCTTCAGCGCCCTGGAGCCCCTGGTGGACCTGCCCATCG | |
| | GCATCAACATCACCAGGTTCCAGACCCTGCTGGCCCTGCACAGGAG | |
| | CTACCTGACCCCCGGCGACAGCAGCAGCGGCTGGACCGCCGGCGC | |
| | CGCCGCCTACTACGTGGGCTACCTGCAGCCCAGGACCTTCCTGCTG | |
| | AAGTACAACGAGAACGGCACCATCACCGACGCCGTGGACTGCGCCC | |
| | TGGACCCCCTGAGCGAGACCAAGTGCACCCTGAAGAGCTTCACCGT | |
| | GGAGAAGGGCATCTACCAGACCAGCAACTTCAGGGTGCAGCCCACC | |
| | GAGAGCATCGTGAGGTTCCCCAACATCACCAACCTGTGCCCCTTCG | |
| | GCGAGGTGTTCAACGCCACCAGGTTCGCCAGCGTGTACGCCTGGAA | |
| | CAGGAAGAGGATCAGCAACTGCGTGGCCGACTACAGCGTGCTGTAC | |
| | AACAGCGCCAGCTTCAGCACCTTCAAGTGCTACGGCGTGAGCCCCA | |
| | CCAAGCTGAACGACCTGTGCTTCACCAACGTGTACGCCGACAGCTTC | |
| | GTGATCAGGGGCGACGAGGTGAGGCAGATCGCCCCCGGCCAGACC | |
| | GGCAAGATCGCCGACTACAACTACAAGCTGCCCGACGACTTCACCG | |
| | GCTGCGTGATCGCCTGGAACAGCAACAACCTGGACAGCAAGGTGGG | |
| | CGGCAACTACAACTACCTGTACAGGCTGTTCAGGAAGAGCAACCTGA | |
| | AGCCCTTCGAGAGGGACATCAGCACCGAGATCTACCAGGCCGGCAG | |
| | CACCCCCTGCAACGGCGTGGAGGGCTTCAACTGCTACTTCCCCCTG | |
| | CAGAGCTACGGCTTCCAGCCCACCAACGGCGTGGGCTACCAGCCCT | |
| | ACAGGGTGGTGGTGCTGAGCTTCGAGCTGCTGCACGCCCCCGCCAC | |
| | CGTGTGCGGCCCCAAGAAGAGCACCAACCTGGTGAAGAACAAGTGC | |
| | GTGAACTTCAACTTCAACGGCCTGACCGGCACCGGCGTGCTGACCG | |
| | AGAGCAACAAGAAGTTCCTGCCCTTCCAGCAGTTCGGCAGGGACAT | |
| | CGCCGACACCACCGACGCCGTGAGGGACCCCCAGACCCTGGAGAT | |
| | CCTGGACATCACCCCCTGCAGCTTCGGCGGCGTGAGCGTGATCACC | |
| | CCCGGCACCAACACCAGCAACCAGGTGGCCGTGCTGTACCAGGACG | |
| | TGAACTGCACCGAGGTGCCCGTGGCCATCCACGCCGACCAGCTGAC | |
| | CCCCACCTGGAGGGTGTACAGCACCGGCAGCAACGTGTTCCAGACC | |
| | AGGGCCGGCTGCCTGATCGGCGCCGAGCACGTGAACAACAGCTAC | |
| | GAGTGCGACATCCCCATCGGCGCCGGCATCTGCGCCAGCTACCAGA | |
| | CCCAGACCAACAGCCCCGGCAGCGCCAGCAGCGTGGCCAGCCAGA | |
| | GCATCATCGCCTACACCATGAGCCTGGGCGCCGAGAACAGCGTGGC | |
| | CTACAGCAACAACAGCATCGCCATCCCCACCAACTTCACCATCAGCG | |
| | TGACCACCGAGATCCTGCCCGTGAGCATGACCAAGACCAGCGTGGA | |
| | CTGCACCATGTACATCTGCGGCGACAGCACCGAGTGCAGCAACCTG | |
| | CTGCTGCAGTACGGCAGCTTCTGCACCCAGCTGAACAGGGCCCTGA | |
| | CCGGCATCGCCGTGGAGCAGGACAAGAACACCCAGGAGGTGTTCGC | |
| | CCAGGTGAAGCAGATCTACAAGACCCCCCCCATCAAGGACTTCGGC | |
| | GGCTTCAACTTCAGCCAGATCCTGCCCGACCCCAGCAAGCCCAGCA | |
| | AGAGGAGCCCCATCGAGGACCTGCTGTTCAACAAGGTGACCCTGGC | |
| | CGACGCCGGCTTCATCAAGCAGTACGGCGACTGCCTGGGCGACATC | |
| | GCCGCCAGGGACCTGATCTGCGCCCAGAAGTTCAACGGCCTGACCG | |
| | TGCTGCCCCCCCTGCTGACCGACGAGATGATCGCCCAGTACACCAG | |
| | CGCCCTGCTGGCCGGCACCATCACCAGCGGCTGGACCTTCGGCGC | |
| | CGGCCCCGCCCTGCAGATCCCCTTCCCCATGCAGATGGCCTACAGG | |
| | TTCAACGGCATCGGCGTGACCCAGAACGTGCTGTACGAGAACCAGA | |
| | AGCTGATCGCCAACCAGTTCAACAGCGCCATCGGCAAGATCCAGGA | |
| | CAGCCTGAGCAGCACCCCCAGCGCCCTGGGCAAGCTGCAGGACGT | |
| | GGTGAACCAGAACGCCCAGGCCCTGAACACCCTGGTGAAGCAGCTG | |
| | AGCAGCAACTTCGGCGCCATCAGCAGCGTGCTGAACGACATCCTGA | |
| | GCAGGCTGGACCCCCCCGAGGCCGAGGTGCAGATCGACAGGCTGA | |
| | TCACCGGCAGGCTGCAGAGCCTGCAGACCTACGTGACCCAGCAGCT | |
| | GATCAGGGCCGCCGAGATCAGGGCCAGCGCCAACCTGGCCGCCAC | |
| | CAAGATGAGCGAGTGCGTGCTGGGCCAGAGCAAGAGGGTGGACTTC | |
| | TGCGGCAAGGGCTACCACCTGATGAGCTTCCCCCAGAGCGCCCCCC | |
| | ACGGCGTGGTGTTCCTGCACGTGACCTACGTGCCCGCCCAGGAGAA | |
| | GAACTTCACCACCGCCCCCGCCATCTGCCACGACGGCAAGGCCCAC | |
| | TTCCCCAGGGAGGGCGTGTTCGTGAGCAACGGCACCCACTGGTTCG | |
| | TGACCCAGAGGAACTTCTACGAGCCCCAGATCATCACCACCGACAAC | |
| | ACCTTCGTGAGCGGCAACTGCGACGTGGTGATCGGCATCGTGAACA | |
| | ACACCGTGTACGACCCCCTGCAGCCCGAGCTGGACAGCTTCAAGGA | |
| | GGAGCTGGACAAGTACTTCAAGAACCACACCAGCCCCGACGTGGAC | |
| | CTGGGCGACATCAGCGGCATCAACGCCAGCGTGGTGAACATCCAGA | |
| | AGGAGATCGACAGGCTGAACGAGGTGGCCAAGAACCTGAACGAGAG | |
| | CCTGATCGACCTGCAGGAGCTGGGCAAGTACGAGCAGTACATCAAG | |
| | TGGCCCTGGTACATCTGGCTGGGCTTCATCGCCGGCCTGATCGCCA | |
| | TCGTGATGGTGACCATCATGCTGTGCTGCATGACCAGCTGCTGCAGC | |
| | TGCCTGAAGGGCTGCTGCAGCTGCGGCAGCTGCTGCAAGTTCGACG | |
| | AGGACGACAGCGAGCCCGTGCTGAAGGGCGTGAAGCTGCACTACAC | |
| | CGGAAGCGGAGCCACGAACTTCTCTCTGTTAAAGCAAGCAGGAGAT | |
| | GTTGAAGAAAACCCCGGGCCTATGAAGGCAATACTAGTAGTTCTGCT | |
| | ATATACATTTGCAACCGCAAATGCAGACACATTATGTATAGGTTATCA | |
| | TGCGAACAACTCAACAGACACTGTAGACACAGTACTAGAAAAGAATG | |
| | TAACAGTAACACACTCTGTTAACCTTCTAGAAGACAAGCATAACGGGA | |
| | AACTATGCAAACTAAGAGGGGTAGCCCCATTGCATTTGGGTAAATGT | |
| | AACATTGCTGGCTGGATCCTGGGAAATCCAGAGTGTGAATCACTCTC | |
| | CACAGCAAGCTCATGGTCCTACATTGTGGAAACATCTAGTTCAGACA | |
| | ATGGAACGTGTTACCCAGGAGATTTCATCGATTATGAGGAGCTAAGA | |
| | GAGCAATTGAGCTCAGTGTCATCATTTGAAAGGTTTGAGATATTCCCC | |
| | AAGACAAGTTCATGGCCCAATCATGACTCGAACAAAGGTGTAACGAC | |
| | AGCATGTCCTCATGCTGGAGCAAAAAGCTTCTACAAAAATTTAATATG | |
| | GCTAGTTAAAAAAGGAAATTCATACCCAAAGCTCAGCAAATCCTACAT | |
| | TAATGATAAAGGGAAAGAAGTCCTCGTGCTATGGGGCATTCACCATC | |
| | CACCTACTAGTGCTGACCAACAAAGTCTCTATCAGAATGCAGATGCA | |
| | TATGTTTTTGTGGGGACATCAAGATACAGCAAGAAGTTCAAGCCGGA | |
| | AATAGCAATAAGGCCCAAAGTGAGGGATCAAGAAGGGAGAATGAACT | |
| | ATTACTGGACACTAGTAGAGCCGGGAGACAAAATAACATTCGAAGCA | |
| | ACTGGAAATCTAGTGGTACCGAGATATGCATTCGCAATGGAAAGAAA | |
| | TGCTGGATCTGGTATTATCATTTCAGATACACCAGTCCACGATTGCAA | |
| | TACAACTTGTCAGACACCCAAGGGTGCTATAAACACCAGCCTCCCAT | |
| | TTCAGAACATACATCCGATCACAATTGGAAAATGTCCAAAATATGTAA | |
| | AAAGCACAAAATTGAGACTGGCCACAGGATTGAGGAATGTCCCGTCC | |
| | ATTCAATCTAGAGGCCTATTTGGGGCCATTGCCGGTTTCATTGAAGG | |
| | GGGGTGGACAGGGATGGTAGATGGATGGTACGGTTATCACCATCAA | |
| | AATGAGCAGGGGTCAGGATATGCAGCCGACCTGAAGAGCACACAGA | |
| | ATGCCATTGACGAGATTACTAACAAAGTAAACTCTGTTATTGAAAAAA | |
| | TGAATACACAGTTCACAGCAGTAGGTAAAGAGTTCAACCACCTGGAA | |
| | AAAAGAATAGAGAATTTAAATAAAAAAGTTGATGATGGTTTCCTGGAC | |
| | ATTTGGACTTACAATGCCGAACTGTTGGTTCTATTGGAAAATGAAAGA | |
| | ACTTTGGACTACCACGATTCAAAGGTGAAGAACTTATATGAAAAGGTA | |
| | AGAAGCCAGTTAAAAAACAATGCCAAGGAAATTGGAAACGGCTGCTT | |
| | TGAATTTTACCACAAATGTGATAACACGTGCATGGAAAGTGTCAAAAA | |
| | TGGGACTTATGACTACCCAAAATACTCAGAGGAAGCAAAATTAAACA | |
| | GAGAAGAAATAGATGGGGTAAAGCTGGAATCAACAAGGATTTACCAG | |
| | ATTTTGGCGATCTATTCAACCGTCGCCAGTTCATTGGTACTGGTAGTC | |
| | TCCCTGGGGGCAATCAGTTTCTGGATGTGCTCTAATGGGTCTCTACA | |
| | GTGTAGAATATGTATTTAAGGAAGCGGAGCCACGAACTTCTCTCTGTT | |
| | AAAGCAAGCAGGAGATGTTGAAGAAAACCCCGGGCCTAGCAAAAGC | |
| | AGGGGATAATTCTATTAACCATGAAGACTATCATTGCTTTGAGCTACA | |
| | TTCTATGTCTGGTTTTCGCTCAAAAACTTCCTGGAAATGACAATAGCA | |
| | CTGCAACGCTGTGCCTTGGGCACCATGCAGTACCAAACGGAACGAT | |
| | AGTGAAAACAATCACGAATGACCAAATTGAAGTTACTAATGCTACTGA | |
| | GCTGGTTCAGAATTCCTCAATAGGTGAAATATGCGACAGTCCTCATC | |
| | AGATCCTTGATGGAGAAAACTGCACACTAATAGATGCTCTATTGGGA | |
| | GACCCTCAGTGTGATGGCTTTCAAAATAAGAAATGGGACCTTTTTGTT | |
| | GAACGAAGCAAAGCCTACAGCAACTGTTACCCTTATGATGTGCCGGA | |
| | TTATGCCTCCCTTAGGTCACTAGTTGCCTCATCCGGCACACTGGAGT | |
| | TTAACAATGAAAGCTTCAATTGGGCTGGAGTCACTCAAAACGGAACA | |
| | AGTTCTGCTTGCATAAGGGGATCTAATAGTAGTTTCTTTAGTAGATTA | |
| | AATTGGTTGACCCACTTAAACTTCAAGTACCCAGCATTGAACGTGACT | |
| | ATGCCAAACAATGAACAATTTGACAAATTGTACATTTGGGGGGTTCAC | |
| | CACCCGGGTACGGACAAGGACCAAATCTTCCTGTATGCTCAATCATC | |
| | AGGAAGAATCACAGTATCTACCAAAAGAAGCCAACAAGCTGTAATCC | |
| | CGAATATCGGATCTAGACCCAGAATAAGGAATATCCCTAGCAGAATA | |
| | AGCATCTATTGGACAATAGTAAAACCGGGAGACATACTTTTGATTAAC | |
| | AGCACAGGGAATCTAATTGCTCCTAGGGGTTACTTCAAAATACGAAG | |
| | TGGGAAAAGCTCAATAATGAGATCAGATGCACCCATTGGCAAATGCA | |
| | AGTCTGAATGCATCACTCCAAATGGAAGCATTCCCAATGACAAACCAT | |
| | TCCAAAATGTAAACAGGATCACATACGGGGCCTGTCCCAGATATGTT | |
| | AAGCAAAGCACTCTGAAATTGGCAACAGGAATGCGAAATGTACCAGA | |
| | GAAACAAACTAGAGGCATATTTGGCGCAATAGCGGGTTTCATAGAAA | |
| | ATGGTTGGGAGGGAATGGTGGATGGTTGGTACGGTTTCAGGCATCA | |
| | AAATTCTGAGGGAAGAGGACAAGCAGCAGATCTCAAAAGCACTCAAG | |
| | CAGCAATCGATCAAATCAATGGGAAGCTGAATCGATTGATCGGGAAA | |
| | ACCAACGAGAAATTCCATCAGATTGAGAAAGAATTCTCAGAAGTAGAA | |
| | GGGAGAATTCAGGACCTTGAGAAATATGTTGAGGACACAAAAATAGA | |
| | TCTCTGGTCATACAACGCAGAGCTTCTTGTTGCCCTGGAAAACCAAC | |
| | ATACAATTGATCTAACTGACTCAGAAATGAACAAACTGTTTGAAAAAA | |
| | CAAAGAAGCAACTGAGGGAAAATGCTGAGGATATGGGCAATGGTTGT | |
| | TTCAAAATATACCACAAATGTGACAATGCCTGCATAGGATCAATCAGA | |
| | AATGGAACTTATGACCACGATGTATACAGGGATGAAGCATTAAACAA | |
| | CCGGTTCCAGATCAAGGGAGTTGAGCTGAAGTCAGGGTACAAAGATT | |
| | GGATCCTATGGATTTCCTTTGCCATATCATGTTTTTTGCTTTGTGTTGC | |
| | TTTGTTGGGGTTCATCATGTGGGCCTGCCAAAAGGGCAACATTAGGT | |
| | GCAACATTTGCATTTGAGTGCATTAATTAAAAACACCCTTGTTTCTACT | |
| | GGAAGCGGAGCCACGAACTTCTCTCTGTTAAAGCAAGCAGGAGATGT | |
| | TGAAGAAAACCCCGGGCCTATTTTGTAATATCCACAAAATGAAGGCAA | |
| | TAATTGTACTACTCATGGTAGTAACATCCAATGCAGATCGAATCTGCA | |
| | CTGGGATAACATCGTCAAACTCACCACATGTCGTCAAAACTGCTACT | |
| | CAAGGGGAGGTCAACGTGACCGGTGTAATACCACTGACAACAACAC | |
| | CCACCAAATCTCATTTTGCAAATCTCAAAGGAACAGAAACCAGGGGG | |
| | AAACTATGCCCAAAATGCCTCAACTGCACAGATCTGGATGTAGCCTT | |
| | GGGCAGACCAAAATGCACAGGGAAAATACCCTCTGCAAGGGTTTCAA | |
| | TACTCCATGAAGTCAGACCTGTTACATCTGGGTGCTTTCCTATAATGC | |
| | ACGATAGAACAAAAATTAGACAGCTGCCTAACCTTCTCCGAGGATAC | |
| | GAACATGTCAGGTTATCAACTCACAACGTTATCAATGCAAAAGATGCA | |
| | CCAGGAAGACCCTACGAAATTGGAACCTCAGGGTCTTGCCCTAACAT | |
| | TACCAATGGAAACGGATTCTTCGCAACAATGGCTTGGGCCGTCCCAA | |
| | AAAACAAAACAGCAACAAATCCATTAAGAATAGAAGTACCATACATTT | |
| | GTACAGAAGGAGAAGACCAAATTACCGTTTGGGGGTTCCACTCTGAC | |
| | AACGAGACCCAAATGGCAAAGCTCTATGGGGACTCAAAGCCCCAGA | |
| | AGTTCACCTCATCTGCCAACGGAGTGACCACACATTACGTTTCACAG | |
| | ATTGGTGGCTTCCCAAATCAAACAGAAGACGGAGGACTACCACAAAG | |
| | TGGCAGAATTGTTGTTGATTACATGGTGCAGAAATCTGGAAAAACAG | |
| | GAACAATTACCTATCAAAGAGGTATTTTATTGCCTCAAAAAGTGTGGT | |
| | GCGCAAGTGGCAGGAGCAAGGTAATAAAAGGATCCTTGCCCTTAATT | |
| | GGAGAAGCAGATTGCCTCCATGAAAAATACGGTGGATTAAACAAAAG | |
| | CAAGCCTTACTACACAGGGGAACATGCAAAGGCCATAGGAAATTGCC | |
| | CAATATGGGTGAAAACACCCTTGAAGCTGGCCAATGGAACCAAATAT | |
| | AGACCCCCTGCAAAACTATTAAAGGAAAGAGGTTTCTTCGGAGCCAT | |
| | TGCTGGTTTCTTAGAGGGAGGATGGGAAGGAATGATTGCAGGTTGG | |
| | CACGGATACACATCCCATGGGGCACATGGAGTAGCGGTGGCAGCTG | |
| | ACCTTAAGAGCACTCAAGAGGCCATAAACAAGATAACAAAAAATCTCA | |
| | ACTCTTTGAGTGAGCTGGAAGTAAAGAATCTTCAAAGACTAAGCGGT | |
| | GCCATGGATGAACTCCACAACGAAATACTAGAACTAGATGAGAAAGT | |
| | GGATGATCTCAGAGCTGACACAATAAGCTCACAAATAGAACTCGCAG | |
| | TCCTGCTTTCCAATGAAGGAATAATAAACAGTGAAGATGAACATCTCT | |
| | TGGCGCTTGAAAGAAAGCTGAAGAAAATGCTGGGCCCCTCTGCTGTA | |
| | GAGATAGGGAATGGATGCTTTGAAACCAAACACAAGTGCAACCAGAC | |
| | CTGCCTCGACAGAATAGCTGCTGGTACCTTTGATGCAGGAGAATTTT | |
| | CTCTCCCCACCTTTGATTCACTGAATATTACTGCTGCATCTTTAAATG | |
| | ACGACGGATTGGACAATCATACTATACTGCTTTACTACTCAACTGCTG | |
| | CCTCCAGTTTGGCTGTAACACTGATGATAGCTATCTTTGTTGTTTATA | |
| | TGGTCTCCAGAGACAATGTTTCTTGCTCCATTTGTCTATAAGGAAAGT | |
| | TAAGCCCTGTATTTTCCTTTATTGTAGTGCTTGTTTGCTTGTTGTCATT | |
| | ACAAAGAAACGTTATTGAAAAATTGATGACTCGAGCTGGTACTGCATG | |
| | CACGCAATGCTAGCTGCCCCTTTCCCGTCCTGGGTACCCCGAGTCTC | |
| | CCCCGACCTCGGGTCCCAGGTATGCTCCCACCTCCACCTGCCCCAC | |
| | TCACCACCTCTGCTAGTTCCAGACACCTCCCAAGCACGCAGCAATGC | |
| | AGCTCAAAACGCTTAGCCTAGCCACACCCCCACGGGAAACAGCAGT | |
| | GATTAACCTTTAGCAATAAACGAAAGTTTAACTAAGCTATACTAACCC | |
| | CAGGGTTGGTCAATTTCGTGCCAGCCACACCCTGGAGCTAGCAAAAA | |
| | AAA | |
| Pan-CoV-Flu | CTCGACATTGATTATTGACTAGTTATTAATAGTAATCAATTACGGGGT | 410 |
| Vaccine #22 | CATTAGTTCATAGCCCATATATGGAGTTCCGCGTTACATAACTTACGG | |
| CoV-Wuhan | TAAATGGCCCGCCTGGCTGACCGCCCAACGACCCCCGCCCATTGAC | |
| Spike(2 proline | GTCAATAATGACGTATGTTCCCATAGTAACGCCAATAGGGACTTTCCA | |
| substitution)- | TTGACGTCAATGGGTGGAGTATTTACGGTAAACTGCCCACTTGGCAG | |
| Influenza (HA) | TACATCAAGTGTATCATATGCCAAGTACGCCCCCTATTGACGTCAATG | |
| | ACGGTAAATGGCCCGCCTGGCATTATGCCCAGTACATGACCTTATGG | |
| | GACTTTCCTACTTGGCAGTACATCTACGTATTAGTCATCGCTATTACC | |
| | ATGGTCGAGGTGAGCCCCACGTTCTGCTTCACTCTCCCCATCTCCCC | |
| | CCCCTCCCCACCCCCAATTTTGTATTTATTTATTTTTTAATTATTTTGT | |
| | GCAGCGATGGGGGCGGGGGGGGGGGGGGGGCGCGCGCCAGGCG | |
| | GGGCGGGGCGGGGCGAGGGGCGGGGCGGGGCGAGGCGGAGAGG | |
| | TGCGGCGGCAGCCAATCAGAGCGGCGCGCTCCGAAAGTTTCCTTTT | |
| | ATGGCGAGGCGGCGGCGGCGGCGGCCCTATAAAAAGCGAAGCGCG | |
| | CGGCGGGCGGGAGTCGCTGCGCGCTGCCTTCGCCCCGTGCCCCGC | |
| | TCCGCCGCCGCCTCGCGCCGCCCGCCCCGGCTCTGACTGACCGCG | |
| | TTACTCCCACAGGTGAGCGGGCGGGACGGCCCTTCTCCTCCGGGCT | |
| | GTAATTAGCGCTTGGTTTAATGACGGCTTGTTTCTTTTCTGTGGCTGC | |
| | GTGAAAGCCTTGAGGGGCTCCGGGAGGGCCCTTTGTGCGGGGGGA | |
| | GCGGCTCGGGGGGTGCGTGCGTGTGTGTGTGCGTGGGGAGCGCCG | |
| | CGTGCGGCTCCGCGCTGCCCGGCGGCTGTGAGCGCTGCGGGCGCG | |
| | GCGCGGGGCTTTGTGCGCTCCGCAGTGTGCGCGAGGGGAGCGCGG | |
| | CCGGGGGCGGTGCCCCGCGGTGCGGGGGGGGCTGCGAGGGGAAC | |
| | AAAGGCTGCGTGCGGGGTGTGTGCGTGGGGGGGTGAGCAGGGGGT | |
| | GTGGGCGCGTCGGTCGGGCTGCAACCCCCCCTGCACCCCCCTCCC | |
| | CGAGTTGCTGAGCACGGCCCGGCTTCGGGTGCGGGGCTCCGTACG | |
| | GGGCGTGGCGCGGGGCTCGCCGTGCCGGGCGGGGGGTGGCGGCA | |
| | GGTGGGGGTGCCGGGCGGGGCGGGGCCGCCTCGGGCCGGGGAG | |
| | GGCTCGGGGGAGGGGCGCGGCGGCCCCCGGAGCGCCGGCGGCTG | |
| | TCGAGGCGCGGCGAGCCGCAGCCATTGCCTTTTATGGTAATCGTGC | |
| | GAGAGGGCGCAGGGACTTCCTTTGTCCCAAATCTGTGCGGAGCCGA | |
| | AATCTGGGAGGCGCCGCCGCACCCCCTCTAGCGGGCGCGGGGCGA | |
| | AGCGGTGCGGCGCCGGCAGGAAGGAAATGGGCGGGGAGGGCCTTC | |
| | GTGCGTCGCCGCGCCGCCGTCCCCTTCTCCCTCTCCAGCCTCGGGG | |
| | CTGTCCGCGGGGGGACGGCTGCCTTCGGGGGGGACGGGGCAGGG | |
| | CGGGGTTCGGCTTCTGGCGTGTGACCGGCGGCTCTAGAGCCTCTGC | |
| | TAACCATGTTCATGCCTTCTTCTTTTTCCTACAGCTCCTGGGCAACGT | |
| | GCTGGTTATTGTGCTGTCTCATCATTTTGGCAAAGAATTGGAGAATAA | |
| | ACTAGTATTCTTCTGGTCCCCACAGACTCAGAGAGAACCCGCCACCA | |
| | TGTTCGTGTTCCTGGTGCTGCTGCCCCTGGTGAGCAGCCAGTGCGT | |
| | GAACCTGACCACCAGGACCCAGCTGCCCCCCGCCTACACCAACAGC | |
| | TTCACCAGGGGCGTGTACTACCCCGACAAGGTGTTCAGGAGCAGCG | |
| | TGCTGCACAGCACCCAGGACCTGTTCCTGCCCTTCTTCAGCAACGTG | |
| | ACCTGGTTCCACGCCATCCACGTGAGCGGCACCAACGGCACCAAGA | |
| | GGTTCGACAACCCCGTGCTGCCCTTCAACGACGGCGTGTACTTCGC | |
| | CAGCACCGAGAAGAGCAACATCATCAGGGGCTGGATCTTCGGCACC | |
| | ACCCTGGACAGCAAGACCCAGAGCCTGCTGATCGTGAACAACGCCA | |
| | CCAACGTGGTGATCAAGGTGTGCGAGTTCCAGTTCTGCAACGACCC | |
| | CTTCCTGGGCGTGTACTACCACAAGAACAACAAGAGCTGGATGGAGA | |
| | GCGAGTTCAGGGTGTACAGCAGCGCCAACAACTGCACCTTCGAGTA | |
| | CGTGAGCCAGCCCTTCCTGATGGACCTGGAGGGCAAGCAGGGCAAC | |
| | TTCAAGAACCTGAGGGAGTTCGTGTTCAAGAACATCGACGGCTACTT | |
| | CAAGATCTACAGCAAGCACACCCCCATCAACCTGGTGAGGGACCTG | |
| | CCCCAGGGCTTCAGCGCCCTGGAGCCCCTGGTGGACCTGCCCATCG | |
| | GCATCAACATCACCAGGTTCCAGACCCTGCTGGCCCTGCACAGGAG | |
| | CTACCTGACCCCCGGCGACAGCAGCAGCGGCTGGACCGCCGGCGC | |
| | CGCCGCCTACTACGTGGGCTACCTGCAGCCCAGGACCTTCCTGCTG | |
| | AAGTACAACGAGAACGGCACCATCACCGACGCCGTGGACTGCGCCC | |
| | TGGACCCCCTGAGCGAGACCAAGTGCACCCTGAAGAGCTTCACCGT | |
| | GGAGAAGGGCATCTACCAGACCAGCAACTTCAGGGTGCAGCCCACC | |
| | GAGAGCATCGTGAGGTTCCCCAACATCACCAACCTGTGCCCCTTCG | |
| | GCGAGGTGTTCAACGCCACCAGGTTCGCCAGCGTGTACGCCTGGAA | |
| | CAGGAAGAGGATCAGCAACTGCGTGGCCGACTACAGCGTGCTGTAC | |
| | AACAGCGCCAGCTTCAGCACCTTCAAGTGCTACGGCGTGAGCCCCA | |
| | CCAAGCTGAACGACCTGTGCTTCACCAACGTGTACGCCGACAGCTTC | |
| | GTGATCAGGGGCGACGAGGTGAGGCAGATCGCCCCCGGCCAGACC | |
| | GGCAAGATCGCCGACTACAACTACAAGCTGCCCGACGACTTCACCG | |
| | GCTGCGTGATCGCCTGGAACAGCAACAACCTGGACAGCAAGGTGGG | |
| | CGGCAACTACAACTACCTGTACAGGCTGTTCAGGAAGAGCAACCTGA | |
| | AGCCCTTCGAGAGGGACATCAGCACCGAGATCTACCAGGCCGGCAG | |
| | CACCCCCTGCAACGGCGTGGAGGGCTTCAACTGCTACTTCCCCCTG | |
| | CAGAGCTACGGCTTCCAGCCCACCAACGGCGTGGGCTACCAGCCCT | |
| | ACAGGGTGGTGGTGCTGAGCTTCGAGCTGCTGCACGCCCCCGCCAC | |
| | CGTGTGCGGCCCCAAGAAGAGCACCAACCTGGTGAAGAACAAGTGC | |
| | GTGAACTTCAACTTCAACGGCCTGACCGGCACCGGCGTGCTGACCG | |
| | AGAGCAACAAGAAGTTCCTGCCCTTCCAGCAGTTCGGCAGGGACAT | |
| | CGCCGACACCACCGACGCCGTGAGGGACCCCCAGACCCTGGAGAT | |
| | CCTGGACATCACCCCCTGCAGCTTCGGCGGCGTGAGCGTGATCACC | |
| | CCCGGCACCAACACCAGCAACCAGGTGGCCGTGCTGTACCAGGACG | |
| | TGAACTGCACCGAGGTGCCCGTGGCCATCCACGCCGACCAGCTGAC | |
| | CCCCACCTGGAGGGTGTACAGCACCGGCAGCAACGTGTTCCAGACC | |
| | AGGGCCGGCTGCCTGATCGGCGCCGAGCACGTGAACAACAGCTAC | |
| | GAGTGCGACATCCCCATCGGCGCCGGCATCTGCGCCAGCTACCAGA | |
| | CCCAGACCAACAGCCCCAGGAGGGCCAGGAGCGTGGCCAGCCAGA | |
| | GCATCATCGCCTACACCATGAGCCTGGGCGCCGAGAACAGCGTGGC | |
| | CTACAGCAACAACAGCATCGCCATCCCCACCAACTTCACCATCAGCG | |
| | TGACCACCGAGATCCTGCCCGTGAGCATGACCAAGACCAGCGTGGA | |
| | CTGCACCATGTACATCTGCGGCGACAGCACCGAGTGCAGCAACCTG | |
| | CTGCTGCAGTACGGCAGCTTCTGCACCCAGCTGAACAGGGCCCTGA | |
| | CCGGCATCGCCGTGGAGCAGGACAAGAACACCCAGGAGGTGTTCGC | |
| | CCAGGTGAAGCAGATCTACAAGACCCCCCCCATCAAGGACTTCGGC | |
| | GGCTTCAACTTCAGCCAGATCCTGCCCGACCCCAGCAAGCCCAGCA | |
| | AGAGGAGCTTCATCGAGGACCTGCTGTTCAACAAGGTGACCCTGGC | |
| | CGACGCCGGCTTCATCAAGCAGTACGGCGACTGCCTGGGCGACATC | |
| | GCCGCCAGGGACCTGATCTGCGCCCAGAAGTTCAACGGCCTGACCG | |
| | TGCTGCCCCCCCTGCTGACCGACGAGATGATCGCCCAGTACACCAG | |
| | CGCCCTGCTGGCCGGCACCATCACCAGCGGCTGGACCTTCGGCGC | |
| | CGGCGCCGCCCTGCAGATCCCCTTCGCCATGCAGATGGCCTACAGG | |
| | TTCAACGGCATCGGCGTGACCCAGAACGTGCTGTACGAGAACCAGA | |
| | AGCTGATCGCCAACCAGTTCAACAGCGCCATCGGCAAGATCCAGGA | |
| | CAGCCTGAGCAGCACCGCCAGCGCCCTGGGCAAGCTGCAGGACGT | |
| | GGTGAACCAGAACGCCCAGGCCCTGAACACCCTGGTGAAGCAGCTG | |
| | AGCAGCAACTTCGGCGCCATCAGCAGCGTGCTGAACGACATCCTGA | |
| | GCAGGCTGGACCCCCCCGAGGCCGAGGTGCAGATCGACAGGCTGA | |
| | TCACCGGCAGGCTGCAGAGCCTGCAGACCTACGTGACCCAGCAGCT | |
| | GATCAGGGCCGCCGAGATCAGGGCCAGCGCCAACCTGGCCGCCAC | |
| | CAAGATGAGCGAGTGCGTGCTGGGCCAGAGCAAGAGGGTGGACTTC | |
| | TGCGGCAAGGGCTACCACCTGATGAGCTTCCCCCAGAGCGCCCCCC | |
| | ACGGCGTGGTGTTCCTGCACGTGACCTACGTGCCCGCCCAGGAGAA | |
| | GAACTTCACCACCGCCCCCGCCATCTGCCACGACGGCAAGGCCCAC | |
| | TTCCCCAGGGAGGGCGTGTTCGTGAGCAACGGCACCCACTGGTTCG | |
| | TGACCCAGAGGAACTTCTACGAGCCCCAGATCATCACCACCGACAAC | |
| | ACCTTCGTGAGCGGCAACTGCGACGTGGTGATCGGCATCGTGAACA | |
| | ACACCGTGTACGACCCCCTGCAGCCCGAGCTGGACAGCTTCAAGGA | |
| | GGAGCTGGACAAGTACTTCAAGAACCACACCAGCCCCGACGTGGAC | |
| | CTGGGCGACATCAGCGGCATCAACGCCAGCGTGGTGAACATCCAGA | |
| | AGGAGATCGACAGGCTGAACGAGGTGGCCAAGAACCTGAACGAGAG | |
| | CCTGATCGACCTGCAGGAGCTGGGCAAGTACGAGCAGTACATCAAG | |
| | TGGCCCTGGTACATCTGGCTGGGCTTCATCGCCGGCCTGATCGCCA | |
| | TCGTGATGGTGACCATCATGCTGTGCTGCATGACCAGCTGCTGCAGC | |
| | TGCCTGAAGGGCTGCTGCAGCTGCGGCAGCTGCTGCAAGTTCGACG | |
| | AGGACGACAGCGAGCCCGTGCTGAAGGGCGTGAAGCTGCACTACAC | |
| | CGGAAGCGGAGCCACGAACTTCTCTCTGTTAAAGCAAGCAGGAGAT | |
| | GTTGAAGAAAACCCCGGGCCTATGAAGGCAATACTAGTAGTTCTGCT | |
| | ATATACATTTGCAACCGCAAATGCAGACACATTATGTATAGGTTATCA | |
| | TGCGAACAACTCAACAGACACTGTAGACACAGTACTAGAAAAGAATG | |
| | TAACAGTAACACACTCTGTTAACCTTCTAGAAGACAAGCATAACGGGA | |
| | AACTATGCAAACTAAGAGGGGTAGCCCCATTGCATTTGGGTAAATGT | |
| | AACATTGCTGGCTGGATCCTGGGAAATCCAGAGTGTGAATCACTCTC | |
| | CACAGCAAGCTCATGGTCCTACATTGTGGAAACATCTAGTTCAGACA | |
| | ATGGAACGTGTTACCCAGGAGATTTCATCGATTATGAGGAGCTAAGA | |
| | GAGCAATTGAGCTCAGTGTCATCATTTGAAAGGTTTGAGATATTCCCC | |
| | AAGACAAGTTCATGGCCCAATCATGACTCGAACAAAGGTGTAACGAC | |
| | AGCATGTCCTCATGCTGGAGCAAAAAGCTTCTACAAAAATTTAATATG | |
| | GCTAGTTAAAAAAGGAAATTCATACCCAAAGCTCAGCAAATCCTACAT | |
| | TAATGATAAAGGGAAAGAAGTCCTCGTGCTATGGGGCATTCACCATC | |
| | CACCTACTAGTGCTGACCAACAAAGTCTCTATCAGAATGCAGATGCA | |
| | TATGTTTTTGTGGGGACATCAAGATACAGCAAGAAGTTCAAGCCGGA | |
| | AATAGCAATAAGGCCCAAAGTGAGGGATCAAGAAGGGAGAATGAACT | |
| | ATTACTGGACACTAGTAGAGCCGGGAGACAAAATAACATTCGAAGCA | |
| | ACTGGAAATCTAGTGGTACCGAGATATGCATTCGCAATGGAAAGAAA | |
| | TGCTGGATCTGGTATTATCATTTCAGATACACCAGTCCACGATTGCAA | |
| | TACAACTTGTCAGACACCCAAGGGTGCTATAAACACCAGCCTCCCAT | |
| | TTCAGAACATACATCCGATCACAATTGGAAAATGTCCAAAATATGTAA | |
| | AAAGCACAAAATTGAGACTGGCCACAGGATTGAGGAATGTCCCGTCC | |
| | ATTCAATCTAGAGGCCTATTTGGGGCCATTGCCGGTTTCATTGAAGG | |
| | GGGGTGGACAGGGATGGTAGATGGATGGTACGGTTATCACCATCAA | |
| | AATGAGCAGGGGTCAGGATATGCAGCCGACCTGAAGAGCACACAGA | |
| | ATGCCATTGACGAGATTACTAACAAAGTAAACTCTGTTATTGAAAAAA | |
| | TGAATACACAGTTCACAGCAGTAGGTAAAGAGTTCAACCACCTGGAA | |
| | AAAAGAATAGAGAATTTAAATAAAAAAGTTGATGATGGTTTCCTGGAC | |
| | ATTTGGACTTACAATGCCGAACTGTTGGTTCTATTGGAAAATGAAAGA | |
| | ACTTTGGACTACCACGATTCAAAGGTGAAGAACTTATATGAAAAGGTA | |
| | AGAAGCCAGTTAAAAAACAATGCCAAGGAAATTGGAAACGGCTGCTT | |
| | TGAATTTTACCACAAATGTGATAACACGTGCATGGAAAGTGTCAAAAA | |
| | TGGGACTTATGACTACCCAAAATACTCAGAGGAAGCAAAATTAAACA | |
| | GAGAAGAAATAGATGGGGTAAAGCTGGAATCAACAAGGATTTACCAG | |
| | ATTTTGGCGATCTATTCAACCGTCGCCAGTTCATTGGTACTGGTAGTC | |
| | TCCCTGGGGGCAATCAGTTTCTGGATGTGCTCTAATGGGTCTCTACA | |
| | GTGTAGAATATGTATTTAAGGAAGCGGAGCCACGAACTTCTCTCTGTT | |
| | AAAGCAAGCAGGAGATGTTGAAGAAAACCCCGGGCCTAGCAAAAGC | |
| | AGGGGATAATTCTATTAACCATGAAGACTATCATTGCTTTGAGCTACA | |
| | TTCTATGTCTGGTTTTCGCTCAAAAACTTCCTGGAAATGACAATAGCA | |
| | CTGCAACGCTGTGCCTTGGGCACCATGCAGTACCAAACGGAACGAT | |
| | AGTGAAAACAATCACGAATGACCAAATTGAAGTTACTAATGCTACTGA | |
| | GCTGGTTCAGAATTCCTCAATAGGTGAAATATGCGACAGTCCTCATC | |
| | AGATCCTTGATGGAGAAAACTGCACACTAATAGATGCTCTATTGGGA | |
| | GACCCTCAGTGTGATGGCTTTCAAAATAAGAAATGGGACCTTTTTGTT | |
| | GAACGAAGCAAAGCCTACAGCAACTGTTACCCTTATGATGTGCCGGA | |
| | TTATGCCTCCCTTAGGTCACTAGTTGCCTCATCCGGCACACTGGAGT | |
| | TTAACAATGAAAGCTTCAATTGGGCTGGAGTCACTCAAAACGGAACA | |
| | AGTTCTGCTTGCATAAGGGGATCTAATAGTAGTTTCTTTAGTAGATTA | |
| | AATTGGTTGACCCACTTAAACTTCAAGTACCCAGCATTGAACGTGACT | |
| | ATGCCAAACAATGAACAATTTGACAAATTGTACATTTGGGGGGTTCAC | |
| | CACCCGGGTACGGACAAGGACCAAATCTTCCTGTATGCTCAATCATC | |
| | AGGAAGAATCACAGTATCTACCAAAAGAAGCCAACAAGCTGTAATCC | |
| | CGAATATCGGATCTAGACCCAGAATAAGGAATATCCCTAGCAGAATA | |
| | AGCATCTATTGGACAATAGTAAAACCGGGAGACATACTTTTGATTAAC | |
| | AGCACAGGGAATCTAATTGCTCCTAGGGGTTACTTCAAAATACGAAG | |
| | TGGGAAAAGCTCAATAATGAGATCAGATGCACCCATTGGCAAATGCA | |
| | AGTCTGAATGCATCACTCCAAATGGAAGCATTCCCAATGACAAACCAT | |
| | TCCAAAATGTAAACAGGATCACATACGGGGCCTGTCCCAGATATGTT | |
| | AAGCAAAGCACTCTGAAATTGGCAACAGGAATGCGAAATGTACCAGA | |
| | GAAACAAACTAGAGGCATATTTGGCGCAATAGCGGGTTTCATAGAAA | |
| | ATGGTTGGGAGGGAATGGTGGATGGTTGGTACGGTTTCAGGCATCA | |
| | AAATTCTGAGGGAAGAGGACAAGCAGCAGATCTCAAAAGCACTCAAG | |
| | CAGCAATCGATCAAATCAATGGGAAGCTGAATCGATTGATCGGGAAA | |
| | ACCAACGAGAAATTCCATCAGATTGAGAAAGAATTCTCAGAAGTAGAA | |
| | GGGAGAATTCAGGACCTTGAGAAATATGTTGAGGACACAAAAATAGA | |
| | TCTCTGGTCATACAACGCAGAGCTTCTTGTTGCCCTGGAAAACCAAC | |
| | ATACAATTGATCTAACTGACTCAGAAATGAACAAACTGTTTGAAAAAA | |
| | CAAAGAAGCAACTGAGGGAAAATGCTGAGGATATGGGCAATGGTTGT | |
| | TTCAAAATATACCACAAATGTGACAATGCCTGCATAGGATCAATCAGA | |
| | AATGGAACTTATGACCACGATGTATACAGGGATGAAGCATTAAACAA | |
| | CCGGTTCCAGATCAAGGGAGTTGAGCTGAAGTCAGGGTACAAAGATT | |
| | GGATCCTATGGATTTCCTTTGCCATATCATGTTTTTTGCTTTGTGTTGC | |
| | TTTGTTGGGGTTCATCATGTGGGCCTGCCAAAAGGGCAACATTAGGT | |
| | GCAACATTTGCATTTGAGTGCATTAATTAAAAACACCCTTGTTTCTACT | |
| | GGAAGCGGAGCCACGAACTTCTCTCTGTTAAAGCAAGCAGGAGATGT | |
| | TGAAGAAAACCCCGGGCCTATTTTCTAATATCCACAAAATGAAGGCAA | |
| | TAATTGTACTACTCATGGTAGTAACATCCAATGCAGATCGAATCTGCA | |
| | CTGGGATAACATCGTCAAACTCACCACATGTCGTCAAAACTGCTACT | |
| | CAAGGGGAGGTCAACGTGACCGGTGTAATACCACTGACAACAACAC | |
| | CCACCAAATCTCATTTTGCAAATCTCAAAGGAACAGAAACCAGGGGG | |
| | AAACTATGCCCAAAATGCCTCAACTGCACAGATCTGGATGTAGCCTT | |
| | GGGCAGACCAAAATGCACAGGGAAAATACCCTCTGCAAGGGTTTCAA | |
| | TACTCCATGAAGTCAGACCTGTTACATCTGGGTGCTTTCCTATAATGC | |
| | ACGATAGAACAAAAATTAGACAGCTGCCTAACCTTCTCCGAGGATAC | |
| | GAACATGTCAGGTTATCAACTCACAACGTTATCAATGCAAAAGATGCA | |
| | CCAGGAAGACCCTACGAAATTGGAACCTCAGGGTCTTGCCCTAACAT | |
| | TACCAATGGAAACGGATTCTTCGCAACAATGGCTTGGGCCGTCCCAA | |
| | AAAACAAAACAGCAACAAATCCATTAACAATAGAAGTACCATACATTT | |
| | GTACAGAAGGAGAAGACCAAATTACCGTTTGGGGGTTCCACTCTGAC | |
| | AACGAGACCCAAATGGCAAAGCTCTATGGGGACTCAAAGCCCCAGA | |
| | AGTTCACCTCATCTGCCAACGGAGTGACCACACATTACGTTTCACAG | |
| | ATTGGTGGCTTCCCAAATCAAACAGAAGACGGAGGACTACCACAAAG | |
| | TGGCAGAATTGTTGTTGATTACATGGTGCAGAAATCTGGAAAAACAG | |
| | GAACAATTACCTATCAAAGAGGTATTTTATTGCCTCAAAAAGTGTGGT | |
| | GCGCAAGTGGCAGGAGCAAGGTAATAAAAGGATCCTTGCCCTTAATT | |
| | GGAGAAGCAGATTGCCTCCATGAAAAATACGGTGGATTAAACAAAAG | |
| | CAAGCCTTACTACACAGGGGAACATGCAAAGGCCATAGGAAATTGCC | |
| | CAATATGGGTGAAAACACCCTTGAAGCTGGCCAATGGAACCAAATAT | |
| | AGACCCCCTGCAAAACTATTAAAGGAAAGAGGTTTCTTCGGAGCCAT | |
| | TGCTGGTTTCTTAGAGGGAGGATGGGAAGGAATGATTGCAGGTTGG | |
| | CACGGATACACATCCCATGGGGCACATGGAGTAGCGGTGGCAGCTG | |
| | ACCTTAAGAGCACTCAAGAGGCCATAAACAAGATAACAAAAAATCTCA | |
| | ACTCTTTGAGTGAGCTGGAAGTAAAGAATCTTCAAAGACTAAGCGGT | |
| | GCCATGGATGAACTCCACAACGAAATACTAGAACTAGATGAGAAAGT | |
| | GGATGATCTCAGAGCTGACACAATAAGCTCACAAATAGAACTCGCAG | |
| | TCCTGCTTTCCAATGAAGGAATAATAAACAGTGAAGATGAACATCTCT | |
| | TGGCGCTTGAAAGAAAGCTGAAGAAAATGCTGGGCCCCTCTGCTGTA | |
| | GAGATAGGGAATGGATGCTTTGAAACCAAACACAAGTGCAACCAGAC | |
| | CTGCCTCGACAGAATAGCTGCTGGTACCTTTGATGCAGGAGAATTTT | |
| | CTCTCCCCACCTTTGATTCACTGAATATTACTGCTGCATCTTTAAATG | |
| | ACGACGGATTGGACAATCATACTATACTGCTTTACTACTCAACTGCTG | |
| | CCTCCAGTTTGGCTGTAACACTGATGATAGCTATCTTTGTTGTTTATA | |
| | TGGTCTCCAGAGACAATGTTTCTTGCTCCATTTGTCTATAAGGAAAGT | |
| | TAAGCCCTGTATTTTCCTTTATTGTAGTGCTTGTTTGCTTGTTGTCATT | |
| | ACAAAGAAACGTTATTGAAAAATTGATGACTCGAGCTGGTACTGCATG | |
| | CACGCAATGCTAGCTGCCCCTTTCCCGTCCTGGGTACCCCGAGTCTC | |
| | CCCCGACCTCGGGTCCCAGGTATGCTCCCACCTCCACCTGCCCCAC | |
| | TCACCACCTCTGCTAGTTCCAGACACCTCCCAAGCACGCAGCAATGC | |
| | AGCTCAAAACGCTTAGCCTAGCCACACCCCCACGGGAAACAGCAGT | |
| | GATTAACCTTTAGCAATAAACGAAAGTTTAACTAAGCTATACTAACCC | |
| | CAGGGTTGGTCAATTTCGTGCCAGCCACACCCTGGAGCTAGCAAAAA | |
| | AAA | |
| Pan-CoV-Flu | ATGTTCGTGTTCCTGGTGCTGCTGCCCCTGGTGAGCAGCCAGTGCG | 411 |
| Vaccine #23 | TGAACCTGATCACCAGGACCCAGAGCTACACCAACAGCTTCACCAGG | |
| CoV- Omicron | GGCGTGTACTACCCCGACAAGGTGTTCAGGAGCAGCGTGCTGCACA | |
| BA.2 | GCACCCAGGACCTGTTCCTGCCCTTCTTCAGCAACGTGACCTGGTTC | |
| Spike(HexaPra)- | CACGCCATCCACGTGAGCGGCACCAACGGCACCAAGAGGTTCGACA | |
| Influenza (HA) | ACCCCGTGCTGCCCTTCAACGACGGCGTGTACTTCGCCAGCACCGA | |
| | GAAGAGCAACATCATCAGGGGCTGGATCTTCGGCACCACCCTGGAC | |
| | AGCAAGACCCAGAGCCTGCTGATCGTGAACAACGCCACCAACGTGG | |
| | TGATCAAGGTGTGCGAGTTCCAGTTCTGCAACGACCCCTTCCTGGAC | |
| | GTGTACTACCACAAGAACAACAAGAGCTGGATGGAGAGCGAGTTCA | |
| | GGGTGTACAGCAGCGCCAACAACTGCACCTTCGAGTACGTGAGCCA | |
| | GCCCTTCCTGATGGACCTGGAGGGCAAGCAGGGCAACTTCAAGAAC | |
| | CTGAGGGAGTTCGTGTTCAAGAACATCGACGGCTACTTCAAGATCTA | |
| | CAGCAAGCACACCCCCATCAACCTGGGCAGGGACCTGCCCCAGGGC | |
| | TTCAGCGCCCTGGAGCCCCTGGTGGACCTGCCCATCGGCATCAACA | |
| | TCACCAGGTTCCAGACCCTGCTGGCCCTGCACAGGAGCTACCTGAC | |
| | CCCCGGCGACAGCAGCAGCGGCTGGACCGCCGGCGCCGCCGCCTA | |
| | CTACGTGGGCTACCTGCAGCCCAGGACCTTCCTGCTGAAGTACAAC | |
| | GAGAACGGCACCATCACCGACGCCGTGGACTGCGCCCTGGACCCC | |
| | CTGAGCGAGACCAAGTGCACCCTGAAGAGCTTCACCGTGGAGAAGG | |
| | GCATCTACCAGACCAGCAACTTCAGGGTGCAGCCCACCGAGAGCAT | |
| | CGTGAGGTTCCCCAACATCACCAACCTGTGCCCCTTCGACGAGGTGT | |
| | TCAACGCCACCAGGTTCGCCAGCGTGTACGCCTGGAACAGGAAGAG | |
| | GATCAGCAACTGCGTGGCCGACTACAGCGTGCTGTACAACTTCGCC | |
| | CCCTTCTTCGCCTTCAAGTGCTACGGCGTGAGCCCCACCAAGCTGAA | |
| | CGACCTGTGCTTCACCAACGTGTACGCCGACAGCTTCGTGATCAGG | |
| | GGCAACGAGGTGAGCCAGATCGCCCCCGGCCAGACCGGCAACATC | |
| | GCCGACTACAACTACAAGCTGCCCGACGACTTCACCGGCTGCGTGA | |
| | TCGCCTGGAACAGCAACAAGCTGGACAGCAAGGTGGGCGGCAACTA | |
| | CAACTACCTGTACAGGCTGTTCAGGAAGAGCAACCTGAAGCCCTTCG | |
| | AGAGGGACATCAGCACCGAGATCTACCAGGCCGGCAACAAGCCCTG | |
| | CAACGGCGTGGCCGGCTTCAACTGCTACTTCCCCCTGAGGAGCTAC | |
| | GGCTTCAGGCCCACCTACGGCGTGGGCCACCAGCCCTACAGGGTG | |
| | GTGGTGCTGAGCTTCGAGCTGCTGCACGCCCCCGCCACCGTGTGCG | |
| | GCCCCAAGAAGAGCACCAACCTGGTGAAGAACAAGTGCGTGAACTT | |
| | CAACTTCAACGGCCTGACCGGCACCGGCGTGCTGACCGAGAGCAAC | |
| | AAGAAGTTCCTGCCCTTCCAGCAGTTCGGCAGGGACATCGCCGACA | |
| | CCACCGACGCCGTGAGGGACCCCCAGACCCTGGAGATCCTGGACAT | |
| | CACCCCCTGCAGCTTCGGCGGCGTGAGCGTGATCACCCCCGGCACC | |
| | AACACCAGCAACCAGGTGGCCGTGCTGTACCAGGGCGTGAACTGCA | |
| | CCGAGGTGCCCGTGGCCATCCACGCCGACCAGCTGACCCCCACCTG | |
| | GAGGGTGTACAGCACCGGCAGCAACGTGTTCCAGACCAGGGCCGG | |
| | CTGCCTGATCGGCGCCGAGTACGTGAACAACAGCTACGAGTGCGAC | |
| | ATCCCCATCGGCGCCGGCATCTGCGCCAGCTACCAGACCCAGACCA | |
| | AGAGCCACCAGCAGGCCCAGAGCGTGGCCAGCCAGAGCATCATCG | |
| | CCTACACCATGAGCCTGGGCGCCGAGAACAGCGTGGCCTACAGCAA | |
| | CAACAGCATCGCCATCCCCACCAACTTCACCATCAGCGTGACCACCG | |
| | AGATCCTGCCCGTGAGCATGACCAAGACCAGCGTGGACTGCACCAT | |
| | GTACATCTGCGGCGACAGCACCGAGTGCAGCAACCTGCTGCTGCAG | |
| | TACGGCAGCTTCTGCACCCAGCTGAAGAGGGCCCTGACCGGCATCG | |
| | CCGTGGAGCAGGACAAGAACACCCAGGAGGTGTTCGCCCAGGTGAA | |
| | GCAGATCTACAAGACCCCCCCCATCAAGTACTTCGGCGGCTTCAACT | |
| | TCAGCCAGATCCTGCCCGACCCCAGCAAGCCCAGCAAGAGGAGCCC | |
| | CATCGAGGACCTGCTGTTCAACAAGGTGACCCTGGCCGACGCCGGC | |
| | TTCATCAAGCAGTACGGCGACTGCCTGGGCGACATCGCCGCCAGGG | |
| | ACCTGATCTGCGCCCAGAAGTTCAACGGCCTGACCGTGCTGCCCCC | |
| | CCTGCTGACCGACGAGATGATCGCCCAGTACACCAGCGCCCTGCTG | |
| | GCCGGCACCATCACCAGCGGCTGGACCTTCGGCGCCGGCCCCGCC | |
| | CTGCAGATCCCCTTCCCCATGCAGATGGCCTACAGGTTCAACGGCAT | |
| | CGGCGTGACCCAGAACGTGCTGTACGAGAACCAGAAGCTGATCGCC | |
| | AACCAGTTCAACAGCGCCATCGGCAAGATCCAGGACAGCCTGAGCA | |
| | GCACCCCCAGCGCCCTGGGCAAGCTGCAGGACGTGGTGAACCACAA | |
| | CGCCCAGGCCCTGAACACCCTGGTGAAGCAGCTGAGCAGCAAGTTC | |
| | GGCGCCATCAGCAGCGTGCTGAACGACATCCTGAGCAGGCTGGACC | |
| | CCCCCGAGGCCGAGGTGCAGATCGACAGGCTGATCACCGGCAGGC | |
| | TGCAGAGCCTGCAGACCTACGTGACCCAGCAGCTGATCAGGGCCGC | |
| | CGAGATCAGGGCCAGCGCCAACCTGGCCGCCACCAAGATGAGCGA | |
| | GTGCGTGCTGGGCCAGAGCAAGAGGGTGGACTTCTGCGGCAAGGG | |
| | CTACCACCTGATGAGCTTCCCCCAGAGCGCCCCCCACGGCGTGGTG | |
| | TTCCTGCACGTGACCTACGTGCCCGCCCAGGAGAAGAACTTCACCA | |
| | CCGCCCCCGCCATCTGCCACGACGGCAAGGCCCACTTCCCCAGGGA | |
| | GGGCGTGTTCGTGAGCAACGGCACCCACTGGTTCGTGACCCAGAGG | |
| | AACTTCTACGAGCCCCAGATCATCACCACCGACAACACCTTCGTGAG | |
| | CGGCAACTGCGACGTGGTGATCGGCATCGTGAACAACACCGTGTAC | |
| | GACCCCCTGCAGCCCGAGCTGGACAGCTTCAAGGAGGAGCTGGACA | |
| | AGTACTTCAAGAACCACACCAGCCCCGACGTGGACCTGGGCGACAT | |
| | CAGCGGCATCAACGCCAGCGTGGTGAACATCCAGAAGGAGATCGAC | |
| | AGGCTGAACGAGGTGGCCAAGAACCTGAACGAGAGCCTGATCGACC | |
| | TGCAGGAGCTGGGCAAGTACGAGCAGTACATCAAGTGGCCCTGGTA | |
| | CATCTGGCTGGGCTTCATCGCCGGCCTGATCGCCATCGTGATGGTG | |
| | ACCATCATGCTGTGCTGCATGACCAGCTGCTGCAGCTGCCTGAAGG | |
| | GCTGCTGCAGCTGCGGCAGCTGCTGCAAGTTCGACGAGGACGACAG | |
| | CGAGCCCGTGCTGAAGGGCGTGAAGCTGCACTACACCGGAAGCGG | |
| | AGCCACGAACTTCTCTCTGTTAAAGCAAGCAGGAGATGTTGAAGAAA | |
| | ACCCCGGGCCTATGAAGGCAATACTAGTAGTTCTGCTATATACATTTG | |
| | CAACCGCAAATGCAGACACATTATGTATAGGTTATCATGCGAACAACT | |
| | CAACAGACACTGTAGACACAGTACTAGAAAAGAATGTAACAGTAACA | |
| | CACTCTGTTAACCTTCTAGAAGACAAGCATAACGGGAAACTATGCAAA | |
| | CTAAGAGGGGTAGCCCCATTGCATTTGGGTAAATGTAACATTGCTGG | |
| | CTGGATCCTGGGAAATCCAGAGTGTGAATCACTCTCCACAGCAAGCT | |
| | CATGGTCCTACATTGTGGAAACATCTAGTTCAGACAATGGAACGTGTT | |
| | ACCCAGGAGATTTCATCGATTATGAGGAGCTAAGAGAGCAATTGAGC | |
| | TCAGTGTCATCATTTGAAAGGTTTGAGATATTCCCCAAGACAAGTTCA | |
| | TGGCCCAATCATGACTCGAACAAAGGTGTAACGACAGCATGTCCTCA | |
| | TGCTGGAGCAAAAAGCTTCTACAAAAATTTAATATGGCTAGTTAAAAA | |
| | AGGAAATTCATACCCAAAGCTCAGGAAATCCTACATTAATGATAAAGG | |
| | GAAAGAAGTCCTCGTGCTATGGGGCATTCACCATCCACCTACTAGTG | |
| | CTGACCAACAAAGTCTCTATCAGAATGCAGATGCATATGTTTTTGTGG | |
| | GGACATCAAGATACAGCAAGAAGTTCAAGCCGGAAATAGCAATAAGG | |
| | CCCAAAGTGAGGGATCAAGAAGGGAGAATGAACTATTACTGGACACT | |
| | AGTAGAGCCGGGAGACAAAATAACATTCGAAGCAACTGGAAATCTAG | |
| | TGGTACCGAGATATGCATTCGCAATGGAAAGAAATGCTGGATCTGGT | |
| | ATTATCATTTCAGATACACCAGTCCACGATTGCAATACAACTTGTCAG | |
| | ACACCCAAGGGTGCTATAAACACCAGCCTCCCATTTCAGAACATACA | |
| | TCCGATCACAATTGGAAAATGTCCAAAATATGTAAAAAGCACAAAATT | |
| | GAGACTGGCCACAGGATTGAGGAATGTCCCGTCCATTCAATCTAGAG | |
| | GCCTATTTGGGGCCATTGCCGGTTTCATTGAAGGGGGGTGGACAGG | |
| | GATGGTAGATGGATGGTACGGTTATCACCATCAAAATGAGCAGGGGT | |
| | CAGGATATGCAGCCGACCTGAAGAGCACACAGAATGCCATTGACGA | |
| | GATTACTAACAAAGTAAACTCTGTTATTGAAAAAATGAATACACAGTTC | |
| | ACAGCAGTAGGTAAAGAGTTCAACCACCTGGAAAAAAGAATAGAGAA | |
| | TTTAAATAAAAAAGTTGATGATGGTTTCCTGGACATTTGGACTTACAAT | |
| | GCCGAACTGTTGGTTCTATTGGAAAATGAAAGAACTTTGGACTACCA | |
| | CGATTCAAAGGTGAAGAACTTATATGAAAAGGTAAGAAGCCAGTTAAA | |
| | AAACAATGCCAAGGAAATTGGAAACGGCTGCTTTGAATTTTACCACAA | |
| | ATGTGATAACACGTGCATGGAAAGTGTCAAAAATGGGACTTATGACT | |
| | ACCCAAAATACTGAGAGGAAGGAAAATTAAACAGAGAAGAAATAGAT | |
| | GGGGTAAAGCTGGAATCAACAAGGATTTACCAGATTTTGGCGATCTA | |
| | TTCAACCGTCGCCAGTTCATTGGTACTGGTAGTCTCCCTGGGGGCAA | |
| | TCAGTTTCTGGATGTGCTCTAATGGGTCTCTACAGTGTAGAATATGTA | |
| | TTTAAGGAAGCGGAGCCACGAACTTCTCTCTGTTAAAGCAAGCAGGA | |
| | GATGTTGAAGAAAACCCCGGGCCTAGCAAAAGCAGGGGATAATTCTA | |
| | TTAACCATGAAGACTATCATTGCTTTGAGCTACATTCTATGTCTGGTTT | |
| | TCGCTCAAAAACTTCCTGGAAATGACAATAGCACTGCAACGCTGTGC | |
| | CTTGGGCACCATGCAGTACCAAACGGAACGATAGTGAAAACAATCAC | |
| | GAATGACCAAATTGAAGTTACTAATGCTACTGAGCTGGTTCAGAATTC | |
| | CTCAATAGGTGAAATATGCGACAGTCCTCATCAGATCCTTGATGGAG | |
| | AAAACTGCACACTAATAGATGCTCTATTGGGAGACCCTCAGTGTGAT | |
| | GGCTTTCAAAATAAGAAATGGGACCTTTTTGTTGAACGAAGCAAAGC | |
| | CTACAGCAACTGTTACCCTTATGATGTGCCGGATTATGCCTCCCTTAG | |
| | GTCACTAGTTGCCTCATCCGGCACACTGGAGTTTAACAATGAAAGCT | |
| | TCAATTGGGCTGGAGTCACTCAAAACGGAACAAGTTCTGCTTGCATA | |
| | AGGGGATCTAATAGTAGTTTCTTTAGTAGATTAAATTGGTTGACCCAC | |
| | TTAAACTTCAAGTACCCAGCATTGAACGTGACTATGCCAAACAATGAA | |
| | CAATTTGACAAATTGTACATTTGGGGGGTTCACCACCCGGGTACGGA | |
| | CAAGGACCAAATCTTCCTGTATGCTCAATCATCAGGAAGAATCACAGT | |
| | ATCTACCAAAAGAAGCCAACAAGCTGTAATCCCGAATATCGGATCTA | |
| | GACCCAGAATAAGGAATATCCCTAGCAGAATAAGCATCTATTGGACA | |
| | ATAGTAAAACCGGGAGACATACTTTTGATTAACAGCACAGGGAATCTA | |
| | ATTGCTCCTAGGGGTTACTTCAAAATACGAAGTGGGAAAAGCTCAAT | |
| | AATGAGATCAGATGCACCCATTGGCAAATGCAAGTCTGAATGCATCA | |
| | CTCCAAATGGAAGCATTCCCAATGACAAACCATTCCAAAATGTAAACA | |
| | GGATCACATACGGGGCCTGTCCCAGATATGTTAAGCAAAGCACTCTG | |
| | AAATTGGCAACAGGAATGCGAAATGTACCAGAGAAACAAACTAGAGG | |
| | CATATTTGGCGCAATAGCGGGTTTCATAGAAAATGGTTGGGAGGGAA | |
| | TGGTGGATGGTTGGTACGGTTTCAGGCATCAAAATTCTGAGGGAAGA | |
| | GGACAAGCAGCAGATCTCAAAAGCACTCAAGCAGCAATCGATCAAAT | |
| | CAATGGGAAGCTGAATCGATTGATCGGGAAAACCAACGAGAAATTCC | |
| | ATCAGATTGAGAAAGAATTCTCAGAAGTAGAAGGGAGAATTCAGGAC | |
| | CTTGAGAAATATGTTGAGGACACAAAAATAGATCTCTGGTCATACAAC | |
| | GCAGAGCTTCTTGTTGCCCTGGAAAACCAACATACAATTGATCTAACT | |
| | GACTCAGAAATGAACAAACTGTTTGAAAAAACAAAGAAGCAACTGAG | |
| | GGAAAATGCTGAGGATATGGGCAATGGTTGTTTCAAAATATACCACA | |
| | AATGTGACAATGCCTGCATAGGATCAATCAGAAATGGAACTTATGAC | |
| | CACGATGTATACAGGGATGAAGCATTAAACAACCGGTTCCAGATCAA | |
| | GGGAGTTGAGCTGAAGTCAGGGTACAAAGATTGGATCCTATGGATTT | |
| | CCTTTGCCATATCATGTTTTTTGCTTTGTGTTGCTTTGTTGGGGTTCAT | |
| | CATGTGGGCCTGCCAAAAGGGCAACATTAGGTGCAACATTTGCATTT | |
| | GAGTGCATTAATTAAAAACACCCTTGTTTCTACTGGAAGCGGAGCCA | |
| | CGAACTTCTCTCTGTTAAAGCAAGCAGGAGATGTTGAAGAAAACCCC | |
| | GGGCCTATTTTCTAATATCCACAAAATGAAGGCAATAATTGTACTACT | |
| | CATGGTAGTAACATCCAATGCAGATCGAATCTGCACTGGGATAACAT | |
| | CGTCAAACTCACCACATGTCGTCAAAACTGCTACTCAAGGGGAGGTC | |
| | AACGTGACCGGTGTAATACCACTGACAACAACACCCACCAAATCTCA | |
| | TTTTGCAAATCTCAAAGGAACAGAAACCAGGGGGAAACTATGCCCAA | |
| | AATGCCTCAACTGCACAGATCTGGATGTAGCCTTGGGCAGACCAAAA | |
| | TGCACAGGGAAAATACCCTCTGCAAGGGTTTCAATACTCCATGAAGT | |
| | CAGACCTGTTACATCTGGGTGCTTTCCTATAATGCACGATAGAACAAA | |
| | AATTAGACAGCTGCCTAACCTTCTCCGAGGATACGAACATGTCAGGT | |
| | TATCAACTCACAACGTTATCAATGCAAAAGATGCACCAGGAAGACCC | |
| | TACGAAATTGGAACCTCAGGGTCTTGCCCTAACATTACCAATGGAAA | |
| | CGGATTCTTCGCAACAATGGCTTGGGCCGTCCCAAAAAACAAAACAG | |
| | CAACAAATCCATTAACAATAGAAGTACCATACATTTGTACAGAAGGAG | |
| | AAGACCAAATTACCGTTTGGGGGTTCCACTCTGACAACGAGACCCAA | |
| | ATGGCAAAGCTCTATGGGGACTCAAAGCCCCAGAAGTTCACCTCATC | |
| | TGCCAACGGAGTGACCACACATTACGTTTCACAGATTGGTGGCTTCC | |
| | CAAATCAAACAGAAGACGGAGGACTACCACAAAGTGGCAGAATTGTT | |
| | GTTGATTACATGGTGCAGAAATCTGGAAAAACAGGAACAATTACCTAT | |
| | CAAAGAGGTATTTTATTGCCTCAAAAAGTGTGGTGCGCAAGTGGCAG | |
| | GAGCAAGGTAATAAAAGGATCCTTGCCCTTAATTGGAGAAGCAGATT | |
| | GCCTCCATGAAAAATACGGTGGATTAAACAAAAGCAAGCCTTACTAC | |
| | ACAGGGGAACATGCAAAGGCCATAGGAAATTGCCCAATATGGGTGAA | |
| | AACACCCTTGAAGCTGGCCAATGGAACCAAATATAGACCCCCTGCAA | |
| | AACTATTAAAGGAAAGAGGTTTCTTCGGAGCCATTGCTGGTTTCTTAG | |
| | AGGGAGGATGGGAAGGAATGATTGCAGGTTGGCACGGATACACATC | |
| | CCATGGGGCACATGGAGTAGCGGTGGCAGCTGACCTTAAGAGCACT | |
| | CAAGAGGCCATAAACAAGATAACAAAAAATCTCAACTCTTTGAGTGAG | |
| | CTGGAAGTAAAGAATCTTCAAAGACTAAGCGGTGCCATGGATGAACT | |
| | CCACAACGAAATACTAGAACTAGATGAGAAAGTGGATGATCTCAGAG | |
| | CTGACACAATAAGCTCACAAATAGAACTCGCAGTCCTGCTTTCCAATG | |
| | AAGGAATAATAAACAGTGAAGATGAACATCTCTTGGCGCTTGAAAGA | |
| | AAGCTGAAGAAAATGCTGGGCCCCTCTGCTGTAGAGATAGGGAATG | |
| | GATGCTTTGAAACCAAACACAAGTGCAACCAGACCTGCCTCGACAGA | |
| | ATAGCTGCTGGTACCTTTGATGCAGGAGAATTTTCTCTCCCCACCTTT | |
| | GATTCACTGAATATTACTGCTGCATCTTTAAATGACGACGGATTGGAC | |
| | AATCATACTATACTGCTTTACTACTCAACTGCTGCCTCCAGTTTGGCT | |
| | GTAACACTGATGATAGCTATCTTTGTTGTTTATATGGTCTCCAGAGAC | |
| | AATGTTTCTTGCTCCATTTGTCTATAAGGAAAGTTAAGCCCTGTATTTT | |
| | CCTTTATTGTAGTGCTTGTTTGCTTGTTGTCATTACAAAGAAACGTTAT | |
| | TGAAAAATGGAAGCGGAGCCACGAACTTCTCTCTGTTAAAGCAAGCA | |
| | GGAGATGTTGAAGAAAACCCCGGGCCTTTCGGAGCTATTGCTGGTTT | |
| | CTTGGAAGGAGGATGGGAAGGAATGATTGCAGGTTGGCACGGATAC | |
| | ACATCTCATGGAGCACATGGAGTAGCAGTGGCAGCAGACCTTAAGA | |
| | GTACCCATGATGA | |
| Pan-CoV-Flu | ATGTTCGTGTTCCTGGTGCTGCTGCCCCTGGTGAGCAGCCAGTGCG | 412 |
| Vaccine #24 | TGAACCTGATCACCAGGACCCAGAGCTACACCAACAGCTTCACCAGG | |
| CoV-Omicron | GGCGTGTACTACCCCGACAAGGTGTTCAGGAGCAGCGTGCTGCACA | |
| BA.2 Spike(2 | GCACCCAGGACCTGTTCCTGCCCTTCTTCAGCAACGTGACCTGGTTC | |
| proline | CACGCCATCCACGTGAGCGGCACCAACGGCACCAAGAGGTTCGACA | |
| substitution)- | ACCCCGTGCTGCCCTTCAACGACGGCGTGTACTTCGCCAGCACCGA | |
| Influenza (HA) | GAAGAGCAACATCATCAGGGGCTGGATCTTCGGCACCACCCTGGAC | |
| | AGCAAGACCCAGAGCCTGCTGATCGTGAACAACGCCACCAACGTGG | |
| | TGATCAAGGTGTGCGAGTTCCAGTTCTGCAACGACCCCTTCCTGGAC | |
| | GTGTACTACCACAAGAACAACAAGAGCTGGATGGAGAGCGAGTTCA | |
| | GGGTGTACAGCAGCGCCAACAACTGCACCTTCGAGTACGTGAGCCA | |
| | GCCCTTCCTGATGGACCTGGAGGGCAAGCAGGGCAACTTCAAGAAC | |
| | CTGAGGGAGTTCGTGTTCAAGAACATCGACGGCTACTTCAAGATCTA | |
| | CAGCAAGCACACCCCCATCAACCTGGGCAGGGACCTGCCCCAGGGC | |
| | TTCAGCGCCCTGGAGCCCCTGGTGGACCTGCCCATCGGCATCAACA | |
| | TCACCAGGTTCCAGACCCTGCTGGCCCTGCACAGGAGCTACCTGAC | |
| | CCCCGGCGACAGCAGCAGCGGCTGGACCGCCGGCGCCGCCGCCTA | |
| | CTACGTGGGCTACCTGCAGCCCAGGACCTTCCTGCTGAAGTACAAC | |
| | GAGAACGGCACCATCACCGACGCCGTGGACTGCGCCCTGGACCCC | |
| | CTGAGCGAGACCAAGTGCACCCTGAAGAGCTTCACCGTGGAGAAGG | |
| | GCATCTACCAGACCAGCAACTTCAGGGTGCAGCCCACCGAGAGCAT | |
| | CGTGAGGTTCCCCAACATCACCAACCTGTGCCCCTTCGACGAGGTGT | |
| | TCAACGCCACCAGGTTCGCCAGCGTGTACGCCTGGAACAGGAAGAG | |
| | GATCAGCAACTGCGTGGCCGACTACAGCGTGCTGTACAACTTCGCC | |
| | CCCTTCTTCGCCTTCAAGTGCTACGGCGTGAGCCCCACCAAGCTGAA | |
| | CGACCTGTGCTTCACCAACGTGTACGCCGACAGCTTCGTGATCAGG | |
| | GGCAACGAGGTGAGCCAGATCGCCCCCGGCCAGACCGGCAACATC | |
| | GCCGACTACAACTACAAGCTGCCCGACGACTTCACCGGCTGCGTGA | |
| | TCGCCTGGAACAGCAACAAGCTGGACAGCAAGGTGGGCGGCAACTA | |
| | CAACTACCTGTACAGGCTGTTCAGGAAGAGCAACCTGAAGCCCTTCG | |
| | AGAGGGACATCAGCACCGAGATCTACCAGGCCGGCAACAAGCCCTG | |
| | CAACGGCGTGGCCGGCTTCAACTGCTACTTCCCCCTGAGGAGCTAC | |
| | GGCTTCAGGCCCACCTACGGCGTGGGCCACCAGCCCTACAGGGTG | |
| | GTGGTGCTGAGCTTCGAGCTGCTGCACGCCCCCGCCACCGTGTGCG | |
| | GCCCCAAGAAGAGCACCAACCTGGTGAAGAACAAGTGCGTGAACTT | |
| | CAACTTCAACGGCCTGACCGGCACCGGCGTGCTGACCGAGAGCAAC | |
| | AAGAAGTTCCTGCCCTTCCAGCAGTTCGGCAGGGACATCGCCGACA | |
| | CCACCGACGCCGTGAGGGACCCCCAGACCCTGGAGATCCTGGACAT | |
| | CACCCCCTGCAGCTTCGGCGGCGTGAGCGTGATCACCCCCGGCACC | |
| | AACACCAGCAACCAGGTGGCCGTGCTGTACCAGGGCGTGAACTGCA | |
| | CCGAGGTGCCCGTGGCCATCCACGCCGACCAGCTGACCCCCACCTG | |
| | GAGGGTGTACAGCACCGGCAGCAACGTGTTCCAGACCAGGGCCGG | |
| | CTGCCTGATCGGCGCCGAGTACGTGAACAACAGCTACGAGTGCGAC | |
| | ATCCCCATCGGCGCCGGCATCTGCGCCAGCTACCAGACCCAGACCA | |
| | AGAGCCACCAGCAGGCCCAGAGCGTGGCCAGCCAGAGCATCATCG | |
| | CCTACACCATGAGCCTGGGCGCCGAGAACAGCGTGGCCTACAGCAA | |
| | CAACAGCATCGCCATCCCCACCAACTTCACCATCAGCGTGACCACCG | |
| | AGATCCTGCCCGTGAGCATGACCAAGACCAGCGTGGACTGCACCAT | |
| | GTACATCTGCGGCGACAGCACCGAGTGCAGCAACCTGCTGCTGCAG | |
| | TACGGCAGCTTCTGCACCCAGCTGAAGAGGGCCCTGACCGGCATCG | |
| | CCGTGGAGCAGGACAAGAACACCCAGGAGGTGTTCGCCCAGGTGAA | |
| | GCAGATCTACAAGACCCCCCCCATCAAGTACTTCGGCGGCTTCAACT | |
| | TCAGCCAGATCCTGCCCGACCCCAGCAAGCCCAGCAAGAGGAGCTT | |
| | CATCGAGGACCTGCTGTTCAACAAGGTGACCCTGGCCGACGCCGGC | |
| | TTCATCAAGCAGTACGGCGACTGCCTGGGCGACATCGCCGCCAGGG | |
| | ACCTGATCTGCGCCCAGAAGTTCAACGGCCTGACCGTGCTGCCCCC | |
| | CCTGCTGACCGACGAGATGATCGCCCAGTACACCAGCGCCCTGCTG | |
| | GCCGGCACCATCACCAGCGGCTGGACCTTCGGCGCCGGCGCCGCC | |
| | CTGCAGATCCCCTTCGCCATGCAGATGGCCTACAGGTTCAACGGCAT | |
| | CGGCGTGACCCAGAACGTGCTGTACGAGAACCAGAAGCTGATCGCC | |
| | AACCAGTTCAACAGCGCCATCGGCAAGATCCAGGACAGCCTGAGCA | |
| | GCACCGCCAGCGCCCTGGGCAAGCTGCAGGACGTGGTGAACCACA | |
| | ACGCCCAGGCCCTGAACACCCTGGTGAAGCAGCTGAGCAGCAAGTT | |
| | CGGCGCCATCAGCAGCGTGCTGAACGACATCCTGAGCAGGCTGGAC | |
| | CCCCCCGAGGCCGAGGTGCAGATCGACAGGCTGATCACCGGCAGG | |
| | CTGCAGAGCCTGCAGACCTACGTGACCCAGCAGCTGATCAGGGCCG | |
| | CCGAGATCAGGGCCAGCGCCAACCTGGCCGCCACCAAGATGAGCG | |
| | AGTGCGTGCTGGGCCAGAGCAAGAGGGTGGACTTCTGCGGCAAGG | |
| | GCTACCACCTGATGAGCTTCCCCCAGAGCGCCCCCCACGGCGTGGT | |
| | GTTCCTGCACGTGACCTACGTGCCCGCCCAGGAGAAGAACTTCACC | |
| | ACCGCCCCCGCCATCTGCCACGACGGCAAGGCCCACTTCCCCAGGG | |
| | AGGGCGTGTTCGTGAGCAACGGCACCCACTGGTTCGTGACCCAGAG | |
| | GAACTTCTACGAGCCCCAGATCATCACCACCGACAACACCTTCGTGA | |
| | GCGGCAACTGCGACGTGGTGATCGGCATCGTGAACAACACCGTGTA | |
| | CGACCCCCTGCAGCCCGAGCTGGACAGCTTCAAGGAGGAGCTGGAC | |
| | AAGTACTTCAAGAACCACACCAGCCCCGACGTGGACCTGGGCGACA | |
| | TCAGCGGCATCAACGCCAGCGTGGTGAACATCCAGAAGGAGATCGA | |
| | CAGGCTGAACGAGGTGGCCAAGAACCTGAACGAGAGCCTGATCGAC | |
| | CTGCAGGAGCTGGGCAAGTACGAGCAGTACATCAAGTGGCCCTGGT | |
| | ACATCTGGCTGGGCTTCATCGCCGGCCTGATCGCCATCGTGATGGT | |
| | GACCATCATGCTGTGCTGCATGACCAGCTGCTGCAGCTGCCTGAAG | |
| | GGCTGCTGCAGCTGCGGCAGCTGCTGCAAGTTCGACGAGGACGACA | |
| | GCGAGCCCGTGCTGAAGGGCGTGAAGCTGCACTACACCGGAAGCG | |
| | GAGCCACGAACTTCTCTCTGTTAAAGCAAGCAGGAGATGTTGAAGAA | |
| | AACCCCGGGCCTATGAAGGCAATACTAGTAGTTCTGCTATATACATTT | |
| | GCAACCGCAAATGCAGACACATTATGTATAGGTTATCATGCGAACAA | |
| | CTCAACAGACACTGTAGACACAGTACTAGAAAAGAATGTAACAGTAA | |
| | CACACTCTGTTAACCTTCTAGAAGACAAGCATAACGGGAAACTATGC | |
| | AAACTAAGAGGGGTAGCCCCATTGCATTTGGGTAAATGTAACATTGC | |
| | TGGCTGGATCCTGGGAAATCCAGAGTGTGAATCACTCTCCACAGCAA | |
| | GCTCATGGTCCTACATTGTGGAAACATCTAGTTCAGACAATGGAACG | |
| | TGTTACCCAGGAGATTTCATCGATTATGAGGAGCTAAGAGAGCAATT | |
| | GAGCTCAGTGTCATCATTTGAAAGGTTTGAGATATTCCCCAAGACAA | |
| | GTTCATGGCCCAATCATGACTCGAACAAAGGTGTAACGACAGCATGT | |
| | CCTCATGCTGGAGCAAAAAGCTTCTACAAAAATTTAATATGGCTAGTT | |
| | AAAAAAGGAAATTCATACCCAAAGCTCAGCAAATCCTACATTAATGAT | |
| | AAAGGGAAAGAAGTCCTCGTGCTATGGGGCATTCACCATCCACCTAC | |
| | TAGTGCTGACCAACAAAGTCTCTATCAGAATGCAGATGCATATGTTTT | |
| | TGTGGGGACATCAAGATACAGCAAGAAGTTCAAGCCGGAAATAGCAA | |
| | TAAGGCCCAAAGTGAGGGATCAAGAAGGGAGAATGAACTATTACTGG | |
| | ACACTAGTAGAGCCGGGAGACAAAATAACATTCGAAGCAACTGGAAA | |
| | TCTAGTGGTACCGAGATATGCATTCGCAATGGAAAGAAATGCTGGAT | |
| | CTGGTATTATCATTTCAGATACACCAGTCCACGATTGCAATACAACTT | |
| | GTCAGACACCCAAGGGTGCTATAAACACCAGCCTCCCATTTCAGAAC | |
| | ATACATCCGATCACAATTGGAAAATGTCCAAAATATGTAAAAAGCACA | |
| | AAATTGAGACTGGCCACAGGATTGAGGAATGTCCCGTCCATTCAATC | |
| | TAGAGGCCTATTTGGGGCCATTGCCGGTTTCATTGAAGGGGGGTGG | |
| | ACAGGGATGGTAGATGGATGGTACGGTTATCACCATCAAAATGAGCA | |
| | GGGGTCAGGATATGCAGCCGACCTGAAGAGCACACAGAATGCCATT | |
| | GACGAGATTACTAACAAAGTAAACTCTGTTATTGAAAAAATGAATACA | |
| | CAGTTCACAGCAGTAGGTAAAGAGTTCAACCACCTGGAAAAAAGAAT | |
| | AGAGAATTTAAATAAAAAAGTTGATGATGGTTTCCTGGACATTTGGAC | |
| | TTACAATGCCGAACTGTTGGTTCTATTGGAAAATGAAAGAACTTTGGA | |
| | CTACCACGATTCAAAGGTGAAGAACTTATATGAAAAGGTAAGAAGCC | |
| | AGTTAAAAAACAATGCCAAGGAAATTGGAAACGGCTGCTTTGAATTTT | |
| | ACCACAAATGTGATAACACGTGCATGGAAAGTGTCAAAAATGGGACT | |
| | TATGACTACCCAAAATACTCAGAGGAAGCAAAATTAAACAGAGAAGAA | |
| | ATAGATGGGGTAAAGCTGGAATCAACAAGGATTTACCAGATTTTGGC | |
| | GATCTATTCAACCGTCGCCAGTTCATTGGTACTGGTAGTCTCCCTGG | |
| | GGGCAATCAGTTTCTGGATGTGCTCTAATGGGTCTCTACAGTGTAGA | |
| | ATATGTATTTAAGGAAGCGGAGCCACGAACTTCTCTCTGTTAAAGCAA | |
| | GCAGGAGATGTTGAAGAAAACCCCGGGCCTAGCAAAAGCAGGGGAT | |
| | AATTCTATTAACCATGAAGACTATCATTGCTTTGAGCTACATTCTATGT | |
| | CTGGTTTTCGCTCAAAAACTTCCTGGAAATGACAATAGCACTGCAAC | |
| | GCTGTGCCTTGGGCACCATGCAGTACCAAACGGAACGATAGTGAAAA | |
| | CAATCACGAATGACCAAATTGAAGTTACTAATGCTACTGAGCTGGTTC | |
| | AGAATTCCTCAATAGGTGAAATATGCGACAGTCCTCATCAGATCCTTG | |
| | ATGGAGAAAACTGCACACTAATAGATGCTCTATTGGGAGACCCTCAG | |
| | TGTGATGGCTTTCAAAATAAGAAATGGGACCTTTTTGTTGAACGAAGC | |
| | AAAGCCTACAGCAACTGTTACCCTTATGATGTGCCGGATTATGCCTC | |
| | CCTTAGGTCACTAGTTGCCTCATCCGGCACACTGGAGTTTAACAATG | |
| | AAAGCTTCAATTGGGCTGGAGTCACTCAAAACGGAACAAGTTCTGCT | |
| | TGCATAAGGGGATCTAATAGTAGTTTCTTTAGTAGATTAAATTGGTTG | |
| | ACCCACTTAAACTTCAAGTACCCAGCATTGAACGTGACTATGCCAAAC | |
| | AATGAACAATTTGACAAATTGTACATTTGGGGGGTTCACCACCCGGG | |
| | TACGGACAAGGACCAAATCTTCCTGTATGCTCAATCATCAGGAAGAA | |
| | TCACAGTATCTACGAAAAGAAGCCAACAAGCTGTAATGCGGAATATC | |
| | GGATCTAGACCCAGAATAAGGAATATCCCTAGCAGAATAAGCATCTA | |
| | TTGGACAATAGTAAAACCGGGAGACATACTTTTGATTAACAGCACAG | |
| | GGAATCTAATTGCTCCTAGGGGTTACTTCAAAATACGAAGTGGGAAA | |
| | AGCTCAATAATGAGATCAGATGCACCCATTGGCAAATGCAAGTCTGA | |
| | ATGCATCACTCCAAATGGAAGCATTCCCAATGACAAACCATTCCAAAA | |
| | TGTAAACAGGATCACATACGGGGCCTGTCCCAGATATGTTAAGCAAA | |
| | GCACTCTGAAATTGGCAACAGGAATGCGAAATGTACCAGAGAAACAA | |
| | ACTAGAGGCATATTTGGCGCAATAGCGGGTTTCATAGAAAATGGTTG | |
| | GGAGGGAATGGTGGATGGTTGGTACGGTTTCAGGCATCAAAATTCTG | |
| | AGGGAAGAGGACAAGCAGCAGATCTCAAAAGCACTCAAGCAGCAAT | |
| | CGATCAAATCAATGGGAAGCTGAATCGATTGATCGGGAAAACCAACG | |
| | AGAAATTCCATCAGATTGAGAAAGAATTCTCAGAAGTAGAAGGGAGA | |
| | ATTCAGGACCTTGAGAAATATGTTGAGGACACAAAAATAGATCTCTGG | |
| | TCATACAACGCAGAGCTTCTTGTTGCCCTGGAAAACCAACATACAATT | |
| | GATCTAACTGACTCAGAAATGAACAAACTGTTTGAAAAAACAAAGAAG | |
| | CAACTGAGGGAAAATGCTGAGGATATGGGCAATGGTTGTTTCAAAAT | |
| | ATACCACAAATGTGACAATGCCTGCATAGGATCAATCAGAAATGGAA | |
| | CTTATGACCACGATGTATACAGGGATGAAGCATTAAACAACCGGTTC | |
| | CAGATCAAGGGAGTTGAGCTGAAGTCAGGGTACAAAGATTGGATCCT | |
| | ATGGATTTCCTTTGCCATATCATGTTTTTTGCTTTGTGTTGCTTTGTTG | |
| | GGGTTCATCATGTGGGCCTGCCAAAAGGGCAACATTAGGTGCAACAT | |
| | TTGCATTTGAGTGCATTAATTAAAAACACCCTTGTTTCTACTGGAAGC | |
| | GGAGCCACGAACTTCTCTCTGTTAAAGCAAGCAGGAGATGTTGAAGA | |
| | AAACCCCGGGCCTATTTTCTAATATCCACAAAATGAAGGCAATAATTG | |
| | TACTACTCATGGTAGTAACATCCAATGCAGATCGAATCTGCACTGGG | |
| | ATAACATCGTCAAACTCACCACATGTCGTCAAAACTGCTACTCAAGG | |
| | GGAGGTCAACGTGACCGGTGTAATACCACTGACAACAACACCCACCA | |
| | AATCTCATTTTGCAAATCTCAAAGGAACAGAAACCAGGGGGAAACTAT | |
| | GCCCAAAATGCCTCAACTGCACAGATCTGGATGTAGCCTTGGGCAGA | |
| | CCAAAATGCACAGGGAAAATACCCTCTGCAAGGGTTTCAATACTCCA | |
| | TGAAGTCAGACCTGTTACATCTGGGTGCTTTCCTATAATGCACGATAG | |
| | AACAAAAATTAGACAGCTGCCTAACCTTCTCCGAGGATACGAACATG | |
| | TCAGGTTATCAACTCACAACGTTATCAATGCAAAAGATGCACCAGGAA | |
| | GACCCTACGAAATTGGAACCTCAGGGTCTTGCCCTAACATTACCAAT | |
| | GGAAACGGATTCTTCGCAACAATGGCTTGGGCCGTCCCAAAAAACAA | |
| | AACAGCAACAAATCCATTAACAATAGAAGTACCATACATTTGTACAGA | |
| | AGGAGAAGACCAAATTACCGTTTGGGGGTTCCACTCTGACAACGAGA | |
| | CCCAAATGGCAAAGCTCTATGGGGACTCAAAGCCCCAGAAGTTCACC | |
| | TCATCTGCCAACGGAGTGACCACACATTACGTTTCACAGATTGGTGG | |
| | CTTCCCAAATCAAACAGAAGACGGAGGACTACCACAAAGTGGCAGAA | |
| | TTGTTGTTGATTACATGGTGCAGAAATCTGGAAAAACAGGAACAATTA | |
| | CCTATCAAAGAGGTATTTTATTGCCTCAAAAAGTGTGGTGCGCAAGT | |
| | GGCAGGAGCAAGGTAATAAAAGGATCCTTGCCCTTAATTGGAGAAGC | |
| | AGATTGCCTCCATGAAAAATACGGTGGATTAAACAAAAGCAAGCCTTA | |
| | CTACACAGGGGAACATGCAAAGGCCATAGGAAATTGCCCAATATGGG | |
| | TGAAAACACCCTTGAAGCTGGCCAATGGAACCAAATATAGACCCCCT | |
| | GCAAAACTATTAAAGGAAAGAGGTTTCTTCGGAGCCATTGCTGGTTT | |
| | CTTAGAGGGAGGATGGGAAGGAATGATTGCAGGTTGGCACGGATAC | |
| | ACATCCCATGGGGCACATGGAGTAGCGGTGGCAGCTGACCTTAAGA | |
| | GCACTCAAGAGGCCATAAAGAAGATAACAAAAAATCTCAACTCTTTGA | |
| | GTGAGCTGGAAGTAAAGAATCTTCAAAGACTAAGCGGTGCCATGGAT | |
| | GAACTCCACAACGAAATACTAGAACTAGATGAGAAAGTGGATGATCT | |
| | CAGAGCTGACACAATAAGCTCACAAATAGAACTCGCAGTCCTGCTTT | |
| | CCAATGAAGGAATAATAAACAGTGAAGATGAACATCTCTTGGCGCTT | |
| | GAAAGAAAGCTGAAGAAAATGCTGGGCCCCTCTGCTGTAGAGATAG | |
| | GGAATGGATGCTTTGAAACCAAACACAAGTGCAACCAGACCTGCCTC | |
| | GACAGAATAGCTGCTGGTACCTTTGATGCAGGAGAATTTTCTCTCCC | |
| | CACCTTTGATTCACTGAATATTACTGCTGCATCTTTAAATGACGACGG | |
| | ATTGGACAATCATACTATACTGCTTTACTACTCAACTGCTGCCTCCAG | |
| | TTTGGCTGTAACACTGATGATAGCTATCTTTGTTGTTTATATGGTCTC | |
| | CAGAGACAATGTTTCTTGCTCCATTTGTCTATAAGGAAAGTTAAGCCC | |
| | TGTATTTTCCTTTATTGTAGTGCTTGTTTGCTTGTTGTCATTACAAAGA | |
| | AACGTTATTGAAAAATGGAAGCGGAGCCACGAACTTCTCTCTGTTAAA | |
| | GCAAGCAGGAGATGTTGAAGAAAACCCCGGGCCTTTCGGAGCTATT | |
| | GCTGGTTTCTTGGAAGGAGGATGGGAAGGAATGATTGCAGGTTGGC | |
| | ACGGATACACATCTCATGGAGCACATGGAGTAGCAGTGGCAGCAGA | |
| | CCTTAAGAGTACCCATGATGA | |
| Pan-CoV-Flu | ATGTTCGTGTTCCTGGTGCTGCTGCCCCTGGTGAGCAGCCAGTGCG | 413 |
| Vaccine #25 | TGAACCTGACCACCAGGACCCAGCTGCCCCCCGCCTACACCAACAG | |
| CoV-Wuhan | CTTCACCAGGGGCGTGTACTACCCCGACAAGGTGTTCAGGAGCAGC | |
| Spike(HexaPro)- | GTGCTGCACAGCACCCAGGACCTGTTCCTGCCCTTCTTCAGCAACGT | |
| Influenza (HA) | GACCTGGTTCCACGCCATCCACGTGAGCGGCACCAACGGCACCAAG | |
| | AGGTTCGACAACCCCGTGCTGCCCTTCAACGACGGCGTGTACTTCG | |
| | CCAGCACCGAGAAGAGCAACATCATCAGGGGCTGGATCTTCGGCAC | |
| | CACCCTGGACAGCAAGACCCAGAGCCTGCTGATCGTGAACAACGCC | |
| | ACCAACGTGGTGATCAAGGTGTGCGAGTTCCAGTTCTGCAACGACCC | |
| | CTTCCTGGGCGTGTACTACCACAAGAACAACAAGAGCTGGATGGAGA | |
| | GCGAGTTCAGGGTGTACAGCAGCGCCAACAACTGCACCTTCGAGTA | |
| | CGTGAGCCAGCCCTTCCTGATGGACCTGGAGGGCAAGCAGGGCAAC | |
| | TTCAAGAACCTGAGGGAGTTCGTGTTCAAGAACATCGACGGCTACTT | |
| | CAAGATCTACAGCAAGCACACCCCCATCAACCTGGTGAGGGACCTG | |
| | CCCCAGGGCTTCAGCGCCCTGGAGCCCCTGGTGGACCTGCCCATCG | |
| | GCATCAACATCACCAGGTTCCAGACCCTGCTGGCCCTGCACAGGAG | |
| | CTACCTGACCCCCGGCGACAGCAGCAGCGGCTGGACCGCCGGCGC | |
| | CGCCGCCTACTACGTGGGCTACCTGCAGCCCAGGACCTTCCTGCTG | |
| | AAGTACAACGAGAACGGCACCATCACCGACGCCGTGGACTGCGCCC | |
| | TGGACCCCCTGAGCGAGACCAAGTGCACCCTGAAGAGCTTCACCGT | |
| | GGAGAAGGGCATCTACCAGACCAGCAACTTCAGGGTGCAGCCCACC | |
| | GAGAGCATCGTGAGGTTCCCCAACATCACCAACCTGTGCCCCTTCG | |
| | GCGAGGTGTTCAACGCCACCAGGTTCGCCAGCGTGTACGCCTGGAA | |
| | CAGGAAGAGGATCAGCAACTGCGTGGCCGACTACAGCGTGCTGTAC | |
| | AACAGCGCCAGCTTCAGCACCTTCAAGTGCTACGGCGTGAGCCCCA | |
| | CCAAGCTGAACGACCTGTGCTTCACCAACGTGTACGCCGACAGCTTC | |
| | GTGATCAGGGGCGACGAGGTGAGGCAGATCGCCCCCGGCCAGACC | |
| | GGCAAGATCGCCGACTACAACTACAAGCTGCCCGACGACTTCACCG | |
| | GCTGCGTGATCGCCTGGAACAGCAACAACCTGGACAGCAAGGTGGG | |
| | CGGCAACTACAACTACCTGTACAGGCTGTTCAGGAAGAGCAACCTGA | |
| | AGCCCTTCGAGAGGGACATCAGCACCGAGATCTACCAGGCCGGCAG | |
| | CACCCCCTGCAACGGCGTGGAGGGCTTCAACTGCTACTTCCCCCTG | |
| | CAGAGCTACGGCTTCCAGCCCACCAACGGCGTGGGCTACCAGCCCT | |
| | ACAGGGTGGTGGTGCTGAGCTTCGAGCTGCTGCACGCCCCCGCCAC | |
| | CGTGTGCGGCCCCAAGAAGAGCACCAACCTGGTGAAGAACAAGTGC | |
| | GTGAACTTCAACTTCAACGGCCTGACCGGCACCGGCGTGCTGACCG | |
| | AGAGCAACAAGAAGTTCCTGCCCTTCCAGCAGTTCGGCAGGGACAT | |
| | CGCCGACACCACCGACGCCGTGAGGGACCCCCAGACCCTGGAGAT | |
| | CCTGGACATCACCCCCTGCAGCTTCGGCGGCGTGAGCGTGATCACC | |
| | CCCGGCACCAACACCAGCAACCAGGTGGCCGTGCTGTACCAGGACG | |
| | TGAACTGCACCGAGGTGCCCGTGGCCATCCACGCCGACCAGCTGAC | |
| | CCCCACCTGGAGGGTGTACAGCACCGGCAGCAACGTGTTCCAGACC | |
| | AGGGCCGGCTGCCTGATCGGCGCCGAGCACGTGAACAACAGCTAC | |
| | GAGTGCGACATCCCCATCGGCGCCGGCATCTGCGCCAGCTACCAGA | |
| | CCCAGACCAACAGCCCCGGCAGCGCCAGCAGCGTGGCCAGCCAGA | |
| | GCATCATCGCCTACACCATGAGCCTGGGCGCCGAGAACAGCGTGGC | |
| | CTACAGCAACAACAGCATCGCCATCCCCACCAACTTCACCATCAGCG | |
| | TGACCACCGAGATCCTGCCCGTGAGCATGACCAAGACCAGCGTGGA | |
| | CTGCACCATGTACATCTGCGGCGACAGCACCGAGTGCAGCAACCTG | |
| | CTGCTGCAGTACGGCAGCTTCTGCACCCAGCTGAACAGGGCCCTGA | |
| | CCGGCATCGCCGTGGAGCAGGACAAGAACACCCAGGAGGTGTTCGC | |
| | CCAGGTGAAGCAGATCTACAAGACCCCCCCCATCAAGGACTTCGGC | |
| | GGCTTCAACTTCAGCCAGATCCTGCCCGACCCCAGCAAGCCCAGCA | |
| | AGAGGAGCCCCATCGAGGACCTGCTGTTCAACAAGGTGACCCTGGC | |
| | CGACGCCGGCTTCATCAAGCAGTACGGCGACTGCCTGGGCGACATC | |
| | GCCGCCAGGGACCTGATCTGCGCCCAGAAGTTCAACGGCCTGACCG | |
| | TGCTGCCCCCCCTGCTGACCGACGAGATGATCGCCCAGTACACCAG | |
| | CGCCCTGCTGGCCGGCACCATCACCAGCGGCTGGACCTTCGGCGC | |
| | CGGCCCCGCCCTGCAGATCCCCTTCCCCATGCAGATGGCCTACAGG | |
| | TTCAACGGCATCGGCGTGACCCAGAACGTGCTGTACGAGAACCAGA | |
| | AGCTGATCGCCAACCAGTTCAACAGCGCCATCGGCAAGATCCAGGA | |
| | CAGCCTGAGCAGCACCCCCAGCGCCCTGGGCAAGCTGCAGGACGT | |
| | GGTGAACCAGAACGCCCAGGCCCTGAACACCCTGGTGAAGCAGCTG | |
| | AGCAGCAACTTCGGCGCCATCAGCAGCGTGCTGAACGACATCCTGA | |
| | GCAGGCTGGACCCCCCCGAGGCCGAGGTGCAGATCGACAGGCTGA | |
| | TCACCGGCAGGCTGCAGAGCCTGCAGACCTACGTGACCCAGCAGCT | |
| | GATCAGGGCCGCCGAGATCAGGGCCAGCGCCAACCTGGCCGCCAC | |
| | CAAGATGAGCGAGTGCGTGCTGGGCCAGAGCAAGAGGGTGGACTTC | |
| | TGCGGCAAGGGCTACCACCTGATGAGCTTCCCCCAGAGCGCCCCCC | |
| | ACGGCGTGGTGTTCCTGCACGTGACCTACGTGCCCGCCCAGGAGAA | |
| | GAACTTCACCACCGCCCCCGCCATCTGCCACGACGGCAAGGCCCAC | |
| | TTCCCCAGGGAGGGCGTGTTCGTGAGCAACGGCACCCACTGGTTCG | |
| | TGACCCAGAGGAACTTCTACGAGCCCCAGATCATCACCACCGACAAC | |
| | ACCTTCGTGAGCGGCAACTGCGACGTGGTGATCGGCATCGTGAACA | |
| | ACACCGTGTACGACCCCCTGCAGCCCGAGCTGGACAGCTTCAAGGA | |
| | GGAGCTGGACAAGTACTTCAAGAACCACACCAGCCCCGACGTGGAC | |
| | CTGGGCGACATCAGCGGCATCAACGCCAGCGTGGTGAACATCCAGA | |
| | AGGAGATCGACAGGCTGAACGAGGTGGCCAAGAACCTGAACGAGAG | |
| | CCTGATCGACCTGCAGGAGCTGGGCAAGTACGAGCAGTACATCAAG | |
| | TGGCCCTGGTACATCTGGCTGGGCTTCATCGCCGGCCTGATCGCCA | |
| | TCGTGATGGTGACCATCATGCTGTGCTGCATGACCAGCTGCTGCAGC | |
| | TGCCTGAAGGGCTGCTGCAGCTGCGGCAGCTGCTGCAAGTTCGACG | |
| | AGGACGACAGCGAGCCCGTGCTGAAGGGCGTGAAGCTGCACTACAC | |
| | CGGAAGCGGAGCCACGAACTTCTCTCTGTTAAAGCAAGCAGGAGAT | |
| | GTTGAAGAAAACCCCGGGCCTATGAAGGCAATACTAGTAGTTCTGCT | |
| | ATATACATTTGCAACCGCAAATGCAGACACATTATGTATAGGTTATCA | |
| | TGCGAACAACTCAACAGACACTGTAGACACAGTACTAGAAAAGAATG | |
| | TAACAGTAACACACTCTGTTAACCTTCTAGAAGACAAGCATAACGGGA | |
| | AACTATGCAAACTAAGAGGGGTAGCCCCATTGCATTTGGGTAAATGT | |
| | AACATTGCTGGCTGGATCCTGGGAAATCCAGAGTGTGAATCACTCTC | |
| | CACAGCAAGCTCATGGTCCTACATTGTGGAAACATCTAGTTCAGACA | |
| | ATGGAACGTGTTACCCAGGAGATTTCATCGATTATGAGGAGCTAAGA | |
| | GAGCAATTGAGCTCAGTGTCATCATTTGAAAGGTTTGAGATATTCCCC | |
| | AAGACAAGTTCATGGCCCAATCATGACTCGAACAAAGGTGTAACGAC | |
| | AGCATGTCCTCATGCTGGAGCAAAAAGCTTCTACAAAAATTTAATATG | |
| | GCTAGTTAAAAAAGGAAATTCATACCCAAAGCTCAGCAAATCCTACAT | |
| | TAATGATAAAGGGAAAGAAGTCCTCGTGCTATGGGGCATTCACCATC | |
| | CACCTACTAGTGCTGACCAACAAAGTCTCTATCAGAATGCAGATGCA | |
| | TATGTTTTTGTGGGGACATCAAGATACAGCAAGAAGTTCAAGCCGGA | |
| | AATAGCAATAAGGCCCAAAGTGAGGGATCAAGAAGGGAGAATGAACT | |
| | ATTACTGGACACTAGTAGAGCCGGGAGACAAAATAACATTCGAAGCA | |
| | ACTGGAAATCTAGTGGTACCGAGATATGCATTCGCAATGGAAAGAAA | |
| | TGCTGGATCTGGTATTATCATTTCAGATACACCAGTCCACGATTGCAA | |
| | TACAACTTGTCAGACACCCAAGGGTGCTATAAACACCAGCCTCCCAT | |
| | TTCAGAACATACATCCGATCACAATTGGAAAATGTCCAAAATATGTAA | |
| | AAAGCACAAAATTGAGACTGGCCACAGGATTGAGGAATGTCCCGTCC | |
| | ATTCAATCTAGAGGCCTATTTGGGGCCATTGCCGGTTTCATTGAAGG | |
| | GGGGTGGACAGGGATGGTAGATGGATGGTACGGTTATCACCATCAA | |
| | AATGAGCAGGGGTCAGGATATGCAGCCGACCTGAAGAGCACACAGA | |
| | ATGCCATTGACGAGATTACTAACAAAGTAAACTCTGTTATTGAAAAAA | |
| | TGAATACACAGTTCACAGCAGTAGGTAAAGAGTTCAACCACCTGGAA | |
| | AAAAGAATAGAGAATTTAAATAAAAAAGTTGATGATGGTTTCCTGGAC | |
| | ATTTGGACTTACAATGCCGAACTGTTGGTTCTATTGGAAAATGAAAGA | |
| | ACTTTGGACTACCACGATTCAAAGGTGAAGAACTTATATGAAAAGGTA | |
| | AGAAGCCAGTTAAAAAACAATGCCAAGGAAATTGGAAACGGCTGCTT | |
| | TGAATTTTACCACAAATGTGATAACACGTGCATGGAAAGTGTCAAAAA | |
| | TGGGACTTATGACTACCGAAAATACTCAGAGGAAGCAAAATTAAACA | |
| | GAGAAGAAATAGATGGGGTAAAGCTGGAATCAACAAGGATTTACCAG | |
| | ATTTTGGCGATCTATTCAACCGTCGCCAGTTCATTGGTACTGGTAGTC | |
| | TCCCTGGGGGCAATCAGTTTCTGGATGTGCTCTAATGGGTCTCTACA | |
| | GTGTAGAATATGTATTTAAGGAAGCGGAGCCACGAACTTCTCTCTGTT | |
| | AAAGCAAGCAGGAGATGTTGAAGAAAACCCCGGGCCTAGCAAAAGC | |
| | AGGGGATAATTCTATTAACCATGAAGACTATCATTGCTTTGAGCTACA | |
| | TTCTATGTCTGGTTTTCGCTCAAAAACTTCCTGGAAATGACAATAGCA | |
| | CTGCAACGCTGTGCCTTGGGCACCATGCAGTACCAAACGGAACGAT | |
| | AGTGAAAACAATCACGAATGACCAAATTGAAGTTACTAATGCTACTGA | |
| | GCTGGTTCAGAATTCCTCAATAGGTGAAATATGCGACAGTCCTCATC | |
| | AGATCCTTGATGGAGAAAACTGCACACTAATAGATGCTCTATTGGGA | |
| | GACCCTCAGTGTGATGGCTTTCAAAATAAGAAATGGGACCTTTTTGTT | |
| | GAACGAAGCAAAGCCTACAGCAACTGTTACCCTTATGATGTGCCGGA | |
| | TTATGCCTCCCTTAGGTCACTAGTTGCCTCATCCGGCACACTGGAGT | |
| | TTAACAATGAAAGCTTCAATTGGGCTGGAGTCACTCAAAACGGAACA | |
| | AGTTCTGCTTGCATAAGGGGATCTAATAGTAGTTTCTTTAGTAGATTA | |
| | AATTGGTTGACCCACTTAAACTTCAAGTACCCAGCATTGAACGTGACT | |
| | ATGCCAAACAATGAACAATTTGACAAATTGTACATTTGGGGGGTTCAC | |
| | CACCCGGGTACGGACAAGGACCAAATCTTCCTGTATGCTCAATCATC | |
| | AGGAAGAATCACAGTATCTACCAAAAGAAGCCAACAAGCTGTAATCC | |
| | CGAATATGGGATCTAGACCCAGAATAAGGAATATCCCTAGCAGAATA | |
| | AGCATCTATTGGACAATAGTAAAACCGGGAGACATACTTTTGATTAAC | |
| | AGCACAGGGAATCTAATTGCTCCTAGGGGTTACTTCAAAATACGAAG | |
| | TGGGAAAAGCTCAATAATGAGATCAGATGCACCCATTGGCAAATGCA | |
| | AGTCTGAATGCATCACTCCAAATGGAAGCATTCCCAATGACAAACCAT | |
| | TCCAAAATGTAAACAGGATCACATACGGGGCCTGTCCCAGATATGTT | |
| | AAGCAAAGCACTCTGAAATTGGCAACAGGAATGCGAAATGTACCAGA | |
| | GAAACAAACTAGAGGCATATTTGGCGCAATAGCGGGTTTCATAGAAA | |
| | ATGGTTGGGAGGGAATGGTGGATGGTTGGTACGGTTTCAGGCATCA | |
| | AAATTCTGAGGGAAGAGGACAAGCAGCAGATCTCAAAAGCACTCAAG | |
| | CAGCAATCGATCAAATCAATGGGAAGCTGAATCGATTGATCGGGAAA | |
| | ACCAACGAGAAATTCCATCAGATTGAGAAAGAATTCTCAGAAGTAGAA | |
| | GGGAGAATTCAGGACCTTGAGAAATATGTTGAGGACACAAAAATAGA | |
| | TCTCTGGTCATACAACGCAGAGCTTCTTGTTGCCCTGGAAAACCAAC | |
| | ATACAATTGATCTAACTGACTCAGAAATGAACAAACTGTTTGAAAAAA | |
| | CAAAGAAGCAACTGAGGGAAAATGCTGAGGATATGGGCAATGGTTGT | |
| | TTCAAAATATACCACAAATGTGACAATGCCTGCATAGGATCAATCAGA | |
| | AATGGAACTTATGACCACGATGTATACAGGGATGAAGCATTAAACAA | |
| | CCGGTTCCAGATCAAGGGAGTTGAGCTGAAGTCAGGGTACAAAGATT | |
| | GGATCCTATGGATTTCCTTTGCCATATCATGTTTTTTGCTTTGTGTTGC | |
| | TTTGTTGGGGTTCATCATGTGGGCCTGCCAAAAGGGCAACATTAGGT | |
| | GCAACATTTGCATTTGAGTGCATTAATTAAAAACACCCTTGTTTCTACT | |
| | GGAAGCGGAGCCACGAACTTCTCTCTGTTAAAGCAAGCAGGAGATGT | |
| | TGAAGAAAACCCCGGGCCTATTTTCTAATATCCACAAAATGAAGGCAA | |
| | TAATTGTACTACTCATGGTAGTAACATCCAATGCAGATCGAATCTGCA | |
| | CTGGGATAACATCGTCAAACTCACCACATGTCGTCAAAACTGCTACT | |
| | CAAGGGGAGGTCAACGTGACCGGTGTAATACCACTGACAACAACAC | |
| | CCACCAAATCTCATTTTGCAAATCTCAAAGGAACAGAAACCAGGGGG | |
| | AAACTATGCCCAAAATGCCTCAACTGCACAGATCTGGATGTAGCCTT | |
| | GGGCAGACCAAAATGCACAGGGAAAATACCCTCTGCAAGGGTTTCAA | |
| | TACTCCATGAAGTCAGACCTGTTACATCTGGGTGCTTTCCTATAATGC | |
| | ACGATAGAACAAAAATTAGACAGCTGCCTAACCTTCTCCGAGGATAC | |
| | GAACATGTCAGGTTATCAACTCACAACGTTATCAATGCAAAAGATGCA | |
| | CCAGGAAGACCCTACGAAATTGGAACCTCAGGGTCTTGCCCTAACAT | |
| | TACCAATGGAAACGGATTCTTCGCAACAATGGCTTGGGCCGTCCCAA | |
| | AAAACAAAACAGCAACAAATCCATTAACAATAGAAGTACCATACATTT | |
| | GTACAGAAGGAGAAGACCAAATTACCGTTTGGGGGTTCCACTCTGAC | |
| | AACGAGACCCAAATGGCAAAGCTCTATGGGGACTCAAAGCCCCAGA | |
| | AGTTCACCTCATCTGCCAACGGAGTGACCACACATTACGTTTCACAG | |
| | ATTGGTGGCTTCCCAAATCAAACAGAAGACGGAGGACTACCACAAAG | |
| | TGGCAGAATTGTTGTTGATTACATGGTGCAGAAATCTGGAAAAACAG | |
| | GAACAATTACCTATCAAAGAGGTATTTTATTGCCTCAAAAAGTGTGGT | |
| | GCGCAAGTGGCAGGAGCAAGGTAATAAAAGGATCCTTGCCCTTAATT | |
| | GGAGAAGCAGATTGCCTCCATGAAAAATACGGTGGATTAAACAAAAG | |
| | CAAGCCTTACTACACAGGGGAACATGCAAAGGCCATAGGAAATTGCC | |
| | CAATATGGGTGAAAACACCCTTGAAGCTGGCCAATGGAACCAAATAT | |
| | AGACCCCCTGCAAAACTATTAAAGGAAAGAGGTTTCTTCGGAGCCAT | |
| | TGCTGGTTTCTTAGAGGGAGGATGGGAAGGAATGATTGCAGGTTGG | |
| | CACGGATACACATCCCATGGGGCACATGGAGTAGCGGTGGCAGCTG | |
| | ACCTTAAGAGCACTCAAGAGGCCATAAACAAGATAACAAAAAATCTCA | |
| | ACTCTTTGAGTGAGCTGGAAGTAAAGAATCTTCAAAGACTAAGCGGT | |
| | GCCATGGATGAACTCCACAACGAAATACTAGAACTAGATGAGAAAGT | |
| | GGATGATCTCAGAGCTGACACAATAAGCTCACAAATAGAACTCGCAG | |
| | TCCTGCTTTCCAATGAAGGAATAATAAACAGTGAAGATGAACATCTCT | |
| | TGGCGCTTGAAAGAAAGCTGAAGAAAATGCTGGGCCCCTCTGCTGTA | |
| | GAGATAGGGAATGGATGCTTTGAAACCAAACACAAGTGCAACCAGAC | |
| | CTGCCTCGACAGAATAGCTGCTGGTACCTTTGATGCAGGAGAATTTT | |
| | CTCTCCCCACCTTTGATTCACTGAATATTACTGCTGCATCTTTAAATG | |
| | ACGACGGATTGGACAATCATACTATACTGCTTTACTACTCAACTGCTG | |
| | CCTCCAGTTTGGCTGTAACACTGATGATAGCTATCTTTGTTGTTTATA | |
| | TGGTCTCCAGAGACAATGTTTCTTGCTCCATTTGTCTATAAGGAAAGT | |
| | TAAGCCCTGTATTTTCCTTTATTGTAGTGCTTGTTTGCTTGTTGTCATT | |
| | ACAAAGAAACGTTATTGAAAAATGGAAGCGGAGCCACGAACTTCTCT | |
| | CTGTTAAAGCAAGCAGGAGATGTTGAAGAAAACCCCGGGCCTTTCGG | |
| | AGCTATTGCTGGTTTCTTGGAAGGAGGATGGGAAGGAATGATTGCAG | |
| | GTTGGCACGGATACACATCTCATGGAGCACATGGAGTAGCAGTGGC | |
| | AGCAGACCTTAAGAGTACCCATGATGA | |
| Pan-CoV-Flu | ATGTTCGTGTTCCTGGTGCTGCTGCCCCTGGTGAGCAGCCAGTGCG | 414 |
| Vaccine #26 | TGAACCTGACCACCAGGACCCAGCTGCCCCCCGCCTACACCAACAG | |
| CoV-Wuhan | CTTCACCAGGGGCGTGTACTACCCCGACAAGGTGTTCAGGAGCAGC | |
| Spike(2 proline | GTGCTGCACAGCACCCAGGACCTGTTCCTGCCCTTCTTCAGCAACGT | |
| substitution)- | GACCTGGTTCCACGCCATCCACGTGAGCGGCACCAACGGCACCAAG | |
| Influenza (HA) | AGGTTCGACAACCCCGTGCTGCCCTTCAACGACGGCGTGTACTTCG | |
| | CCAGCACCGAGAAGAGCAACATCATCAGGGGCTGGATCTTCGGCAC | |
| | CACCCTGGACAGCAAGACCCAGAGCCTGCTGATCGTGAACAACGCC | |
| | ACCAACGTGGTGATCAAGGTGTGCGAGTTCCAGTTCTGCAACGACCC | |
| | CTTCCTGGGCGTGTACTACCACAAGAACAACAAGAGCTGGATGGAGA | |
| | GCGAGTTCAGGGTGTACAGCAGCGCCAACAACTGCACCTTCGAGTA | |
| | CGTGAGCCAGCCCTTCCTGATGGACCTGGAGGGCAAGCAGGGCAAC | |
| | TTCAAGAACCTGAGGGAGTTCGTGTTCAAGAACATCGACGGCTACTT | |
| | CAAGATCTACAGCAAGCACACCCCCATCAACCTGGTGAGGGACCTG | |
| | CCCCAGGGCTTCAGCGCCCTGGAGCCCCTGGTGGACCTGCCCATCG | |
| | GCATCAACATCACCAGGTTCCAGACCCTGCTGGCCCTGCACAGGAG | |
| | CTACCTGACCCCCGGCGACAGCAGCAGCGGCTGGACCGCCGGCGC | |
| | CGCCGCCTACTACGTGGGCTACCTGCAGCCCAGGACCTTCCTGCTG | |
| | AAGTACAACGAGAACGGCACCATCACCGACGCCGTGGACTGCGCCC | |
| | TGGACCCCCTGAGCGAGACCAAGTGCACCCTGAAGAGCTTCACCGT | |
| | GGAGAAGGGCATCTACCAGACCAGCAACTTCAGGGTGCAGCCCACC | |
| | GAGAGCATCGTGAGGTTCCCCAACATCACCAACCTGTGCCCCTTCG | |
| | GCGAGGTGTTCAACGCCACCAGGTTCGCCAGCGTGTACGCCTGGAA | |
| | CAGGAAGAGGATCAGCAACTGCGTGGCCGACTACAGCGTGCTGTAC | |
| | AACAGCGCCAGCTTCAGCACCTTCAAGTGCTACGGCGTGAGCCCCA | |
| | CCAAGCTGAACGACCTGTGCTTCACCAACGTGTACGCCGACAGCTTC | |
| | GTGATCAGGGGCGACGAGGTGAGGCAGATCGCCCCCGGCCAGACC | |
| | GGCAAGATCGCCGACTACAACTACAAGCTGCCCGACGACTTCACCG | |
| | GCTGCGTGATCGCCTGGAACAGCAACAACCTGGACAGCAAGGTGGG | |
| | CGGCAACTACAACTACCTGTACAGGCTGTTCAGGAAGAGCAACCTGA | |
| | AGCCCTTCGAGAGGGACATCAGCACCGAGATCTACCAGGCCGGCAG | |
| | CACCCCCTGCAACGGCGTGGAGGGCTTCAACTGCTACTTCCCCCTG | |
| | CAGAGCTACGGCTTCCAGCCCACCAACGGCGTGGGCTACCAGCCCT | |
| | ACAGGGTGGTGGTGCTGAGCTTCGAGCTGCTGCACGCCCCCGCCAC | |
| | CGTGTGCGGCCCCAAGAAGAGCACCAACCTGGTGAAGAACAAGTGC | |
| | GTGAACTTCAACTTCAACGGCCTGACCGGCACCGGCGTGCTGACCG | |
| | AGAGCAACAAGAAGTTCCTGCCCTTCCAGCAGTTCGGCAGGGACAT | |
| | CGCCGACACCACCGACGCCGTGAGGGACCCCCAGACCCTGGAGAT | |
| | CCTGGACATCACCCCCTGCAGCTTCGGCGGCGTGAGCGTGATCACC | |
| | CCCGGCACCAACACCAGCAACCAGGTGGCCGTGCTGTACCAGGACG | |
| | TGAACTGCACCGAGGTGCCCGTGGCCATCCACGCCGACCAGCTGAC | |
| | CCCCACCTGGAGGGTGTACAGCACCGGCAGCAACGTGTTCCAGACC | |
| | AGGGCCGGCTGCCTGATCGGCGCCGAGCACGTGAACAACAGCTAC | |
| | GAGTGCGACATCCCCATCGGCGCCGGCATCTGCGCCAGCTACCAGA | |
| | CCCAGACCAACAGCCCCAGGAGGGCCAGGAGCGTGGCCAGCCAGA | |
| | GCATCATCGCCTACACCATGAGCCTGGGCGCCGAGAACAGCGTGGC | |
| | CTACAGCAACAACAGCATCGCCATCCCCACCAACTTCACCATCAGCG | |
| | TGACCACCGAGATCCTGCCCGTGAGCATGACCAAGACCAGCGTGGA | |
| | CTGCACCATGTACATCTGCGGCGACAGCACCGAGTGCAGCAACCTG | |
| | CTGCTGCAGTACGGCAGCTTCTGCACCCAGCTGAACAGGGCCCTGA | |
| | CCGGCATCGCCGTGGAGCAGGACAAGAACACCCAGGAGGTGTTCGC | |
| | CCAGGTGAAGCAGATCTACAAGACCCCCCCCATCAAGGACTTCGGC | |
| | GGCTTCAACTTCAGCCAGATCCTGCCCGACCCCAGCAAGCCCAGCA | |
| | AGAGGAGCTTCATCGAGGACCTGCTGTTCAACAAGGTGACCCTGGC | |
| | CGACGCCGGCTTCATCAAGCAGTACGGCGACTGCCTGGGCGACATC | |
| | GCCGCCAGGGACCTGATCTGCGCCCAGAAGTTCAACGGCCTGACCG | |
| | TGCTGCCCCCCCTGCTGACCGACGAGATGATCGCCCAGTACACCAG | |
| | CGCCCTGCTGGCCGGCACCATCACCAGCGGCTGGACCTTCGGCGC | |
| | CGGCGCCGCCCTGCAGATCCCCTTCGCCATGCAGATGGCCTACAGG | |
| | TTCAACGGCATCGGCGTGACCCAGAACGTGCTGTACGAGAACCAGA | |
| | AGCTGATCGCCAACCAGTTCAACAGCGCCATCGGCAAGATCCAGGA | |
| | CAGCCTGAGCAGCACCGCCAGCGCCCTGGGCAAGCTGCAGGACGT | |
| | GGTGAACCAGAACGCCCAGGCCCTGAACACCCTGGTGAAGCAGCTG | |
| | AGCAGCAACTTCGGCGCCATCAGCAGCGTGCTGAACGACATCCTGA | |
| | GCAGGCTGGACCCCCCCGAGGCCGAGGTGCAGATCGACAGGCTGA | |
| | TCACCGGCAGGCTGCAGAGCCTGCAGACCTACGTGACCCAGCAGCT | |
| | GATCAGGGCCGCCGAGATCAGGGCCAGCGCCAACCTGGCCGCCAC | |
| | CAAGATGAGCGAGTGCGTGCTGGGCCAGAGCAAGAGGGTGGACTTC | |
| | TGCGGCAAGGGCTACCACCTGATGAGCTTCCCCCAGAGCGCCCCCC | |
| | ACGGCGTGGTGTTCCTGCACGTGACCTACGTGCCCGCCCAGGAGAA | |
| | GAACTTCACCACCGCCCCCGCCATCTGCCACGACGGCAAGGCCCAC | |
| | TTCCCCAGGGAGGGCGTGTTCGTGAGCAACGGCACCCACTGGTTCG | |
| | TGACCCAGAGGAACTTCTACGAGCCCCAGATCATCACCACCGACAAC | |
| | ACCTTCGTGAGCGGCAACTGCGACGTGGTGATCGGCATCGTGAACA | |
| | ACACCGTGTACGACCCCCTGCAGCCCGAGCTGGACAGCTTCAAGGA | |
| | GGAGCTGGACAAGTACTTCAAGAACCACACCAGCCCCGACGTGGAC | |
| | CTGGGCGACATCAGCGGCATCAACGCCAGCGTGGTGAACATCCAGA | |
| | AGGAGATCGACAGGCTGAACGAGGTGGCCAAGAACCTGAACGAGAG | |
| | CCTGATCGACCTGCAGGAGCTGGGCAAGTACGAGCAGTACATCAAG | |
| | TGGCCCTGGTACATCTGGCTGGGCTTCATCGCCGGCCTGATCGCCA | |
| | TCGTGATGGTGACCATCATGCTGTGCTGCATGACCAGCTGCTGCAGC | |
| | TGCCTGAAGGGCTGCTGCAGCTGCGGCAGCTGCTGCAAGTTCGACG | |
| | AGGACGACAGCGAGCCCGTGCTGAAGGGCGTGAAGCTGCACTACAC | |
| | CGGAAGCGGAGCCACGAACTTCTCTCTGTTAAAGCAAGCAGGAGAT | |
| | GTTGAAGAAAACCCCGGGCCTATGAAGGCAATACTAGTAGTTCTGCT | |
| | ATATACATTTGCAACCGCAAATGCAGACACATTATGTATAGGTTATCA | |
| | TGCGAACAACTCAACAGACACTGTAGACACAGTACTAGAAAAGAATG | |
| | TAACAGTAACACACTCTGTTAACCTTCTAGAAGACAAGCATAACGGGA | |
| | AACTATGCAAACTAAGAGGGGTAGCCCCATTGCATTTGGGTAAATGT | |
| | AACATTGCTGGCTGGATCCTGGGAAATCCAGAGTGTGAATCACTCTC | |
| | CACAGCAAGCTCATGGTCCTACATTGTGGAAACATCTAGTTCAGACA | |
| | ATGGAACGTGTTACCCAGGAGATTTCATCGATTATGAGGAGCTAAGA | |
| | GAGCAATTGAGCTCAGTGTCATCATTTGAAAGGTTTGAGATATTCCCC | |
| | AAGACAAGTTCATGGCCCAATCATGACTCGAACAAAGGTGTAACGAC | |
| | AGCATGTCCTCATGCTGGAGCAAAAAGCTTCTACAAAAATTTAATATG | |
| | GCTAGTTAAAAAAGGAAATTCATACCCAAAGCTCAGCAAATCCTACAT | |
| | TAATGATAAAGGGAAAGAAGTCCTCGTGCTATGGGGCATTCACCATC | |
| | CACCTACTAGTGCTGACCAACAAAGTCTCTATCAGAATGCAGATGCA | |
| | TATGTTTTTGTGGGGACATCAAGATACAGCAAGAAGTTCAAGCCGGA | |
| | AATAGCAATAAGGCCCAAAGTGAGGGATCAAGAAGGGAGAATGAACT | |
| | ATTACTGGACACTAGTAGAGCCGGGAGACAAAATAACATTCGAAGCA | |
| | ACTGGAAATCTAGTGGTACCGAGATATGCATTCGCAATGGAAAGAAA | |
| | TGCTGGATCTGGTATTATCATTTCAGATACACCAGTCCACGATTGCAA | |
| | TACAACTTGTCAGACACCCAAGGGTGCTATAAACACCAGCCTCCCAT | |
| | TTCAGAACATACATCCGATCACAATTGGAAAATGTCCAAAATATGTAA | |
| | AAAGCACAAAATTGAGACTGGCCACAGGATTGAGGAATGTCCCGTCC | |
| | ATTCAATCTAGAGGCCTATTTGGGGCCATTGCCGGTTTCATTGAAGG | |
| | GGGGTGGACAGGGATGGTAGATGGATGGTACGGTTATCACCATCAA | |
| | AATGAGCAGGGGTCAGGATATGCAGCCGACCTGAAGAGCACACAGA | |
| | ATGCCATTGACGAGATTACTAACAAAGTAAACTCTGTTATTGAAAAAA | |
| | TGAATACACAGTTCACAGCAGTAGGTAAAGAGTTCAACCACCTGGAA | |
| | AAAAGAATAGAGAATTTAAATAAAAAAGTTGATGATGGTTTCCTGGAC | |
| | ATTTGGACTTACAATGCCGAACTGTTGGTTCTATTGGAAAATGAAAGA | |
| | ACTTTGGACTACCACGATTCAAAGGTGAAGAACTTATATGAAAAGGTA | |
| | AGAAGCCAGTTAAAAAACAATGCCAAGGAAATTGGAAACGGCTGCTT | |
| | TGAATTTTACCACAAATGTGATAACACGTGCATGGAAAGTGTCAAAAA | |
| | TGGGACTTATGACTACCCAAAATACTCAGAGGAAGCAAAATTAAACA | |
| | GAGAAGAAATAGATGGGGTAAAGCTGGAATCAACAAGGATTTACCAG | |
| | ATTTTGGCGATCTATTCAACCGTCGCCAGTTCATTGGTACTGGTAGTC | |
| | TCCCTGGGGGCAATCAGTTTCTGGATGTGCTCTAATGGGTCTCTACA | |
| | GTGTAGAATATGTATTTAAGGAAGCGGAGCCACGAACTTCTCTCTGTT | |
| | AAAGCAAGCAGGAGATGTTGAAGAAAACCCCGGGCCTAGCAAAAGC | |
| | AGGGGATAATTCTATTAACCATGAAGACTATCATTGCTTTGAGCTACA | |
| | TTCTATGTCTGGTTTTCGCTCAAAAACTTCCTGGAAATGACAATAGCA | |
| | CTGCAACGCTGTGCCTTGGGCACCATGCAGTACCAAACGGAACGAT | |
| | AGTGAAAACAATCACGAATGACCAAATTGAAGTTACTAATGCTACTGA | |
| | GCTGGTTCAGAATTCCTCAATAGGTGAAATATGCGACAGTCCTCATC | |
| | AGATCCTTGATGGAGAAAACTGCACACTAATAGATGCTCTATTGGGA | |
| | GACCCTCAGTGTGATGGCTTTCAAAATAAGAAATGGGACCTTTTTGTT | |
| | GAACGAAGCAAAGCCTACAGCAACTGTTACCCTTATGATGTGCCGGA | |
| | TTATGCCTCCCTTAGGTCACTAGTTGCCTCATCCGGCACACTGGAGT | |
| | TTAACAATGAAAGCTTCAATTGGGCTGGAGTCACTCAAAACGGAACA | |
| | AGTTCTGCTTGCATAAGGGGATCTAATAGTAGTTTCTTTAGTAGATTA | |
| | AATTGGTTGACCCACTTAAACTTCAAGTACCCAGCATTGAACGTGACT | |
| | ATGCCAAACAATGAACAATTTGACAAATTGTACATTTGGGGGGTTCAC | |
| | CACCCGGGTACGGACAAGGACCAAATCTTCCTGTATGCTCAATCATC | |
| | AGGAAGAATCACAGTATCTACCAAAAGAAGCCAACAAGCTGTAATCC | |
| | CGAATATCGGATCTAGACCCAGAATAAGGAATATCCCTAGCAGAATA | |
| | AGCATCTATTGGACAATAGTAAAACCGGGAGACATACTTTTGATTAAC | |
| | AGCACAGGGAATCTAATTGCTCCTAGGGGTTACTTCAAAATACGAAG | |
| | TGGGAAAAGCTCAATAATGAGATCAGATGCACCCATTGGCAAATGCA | |
| | AGTCTGAATGCATCACTCCAAATGGAAGCATTCCCAATGACAAACCAT | |
| | TCCAAAATGTAAACAGGATCACATACGGGGCCTGTCCCAGATATGTT | |
| | AAGCAAAGCACTCTGAAATTGGCAACAGGAATGCGAAATGTACCAGA | |
| | GAAACAAACTAGAGGCATATTTGGCGCAATAGCGGGTTTCATAGAAA | |
| | ATGGTTGGGAGGGAATGGTGGATGGTTGGTACGGTTTCAGGCATCA | |
| | AAATTCTGAGGGAAGAGGACAAGCAGCAGATCTCAAAAGCACTCAAG | |
| | CAGCAATCGATCAAATCAATGGGAAGCTGAATCGATTGATCGGGAAA | |
| | ACCAACGAGAAATTCCATCAGATTGAGAAAGAATTCTCAGAAGTAGAA | |
| | GGGAGAATTCAGGACCTTGAGAAATATGTTGAGGACACAAAAATAGA | |
| | TCTCTGGTCATACAACGCAGAGCTTCTTGTTGCCCTGGAAAACCAAC | |
| | ATACAATTGATCTAACTGACTCAGAAATGAACAAACTGTTTGAAAAAA | |
| | CAAAGAAGCAACTGAGGGAAAATGCTGAGGATATGGGCAATGGTTGT | |
| | TTCAAAATATACCACAAATGTGACAATGCCTGCATAGGATCAATCAGA | |
| | AATGGAACTTATGACCACGATGTATACAGGGATGAAGCATTAAACAA | |
| | CCGGTTCCAGATCAAGGGAGTTGAGCTGAAGTCAGGGTACAAAGATT | |
| | GGATCCTATGGATTTCCTTTGCCATATCATGTTTTTTGCTTTGTGTTGC | |
| | TTTGTTGGGGTTCATCATGTGGGCCTGCCAAAAGGGCAACATTAGGT | |
| | GCAACATTTGCATTTGAGTGCATTAATTAAAAACACCCTTGTTTCTACT | |
| | GGAAGCGGAGCCACGAACTTCTCTCTGTTAAAGCAAGCAGGAGATGT | |
| | TGAAGAAAACCCCGGGCCTATTTTCTAATATCCACAAAATGAAGGCAA | |
| | TAATTGTACTACTCATGGTAGTAACATCCAATGCAGATCGAATCTGCA | |
| | CTGGGATAACATCGTCAAACTCACCACATGTCGTCAAAACTGCTACT | |
| | CAAGGGGAGGTCAACGTGACCGGTGTAATACCACTGACAACAACAC | |
| | CCACCAAATCTCATTTTGCAAATCTCAAAGGAACAGAAACCAGGGGG | |
| | AAACTATGCCCAAAATGCCTCAACTGCACAGATCTGGATGTAGCCTT | |
| | GGGCAGACCAAAATGCACAGGGAAAATACCCTCTGCAAGGGTTTCAA | |
| | TACTCCATGAAGTCAGACCTGTTACATCTGGGTGCTTTCCTATAATGC | |
| | ACGATAGAACAAAAATTAGACAGCTGCCTAACCTTCTCCGAGGATAC | |
| | GAACATGTCAGGTTATCAACTCACAACGTTATCAATGCAAAAGATGCA | |
| | CCAGGAAGACCCTACGAAATTGGAACCTCAGGGTCTTGCCCTAACAT | |
| | TACCAATGGAAACGGATTCTTCGCAACAATGGCTTGGGCCGTCCCAA | |
| | AAAACAAAACAGCAACAAATCCATTAACAATAGAAGTACCATACATTT | |
| | GTACAGAAGGAGAAGACCAAATTACCGTTTGGGGGTTCCACTCTGAC | |
| | AACGAGACCCAAATGGCAAAGCTCTATGGGGACTCAAAGCCCCAGA | |
| | AGTTCACCTCATCTGCCAACGGAGTGACCACACATTACGTTTCACAG | |
| | ATTGGTGGCTTCCCAAATCAAACAGAAGACGGAGGACTACCACAAAG | |
| | TGGCAGAATTGTTGTTGATTACATGGTGCAGAAATCTGGAAAAACAG | |
| | GAACAATTACCTATCAAAGAGGTATTTTATTGCCTCAAAAAGTGTGGT | |
| | GCGCAAGTGGCAGGAGCAAGGTAATAAAAGGATCCTTGCCCTTAATT | |
| | GGAGAAGCAGATTGCCTCCATGAAAAATACGGTGGATTAAACAAAAG | |
| | CAAGCCTTACTACACAGGGGAACATGCAAAGGCCATAGGAAATTGCC | |
| | CAATATGGGTGAAAACACCCTTGAAGCTGGCCAATGGAACCAAATAT | |
| | AGACCCCCTGCAAAACTATTAAAGGAAAGAGGTTTCTTCGGAGCCAT | |
| | TGCTGGTTTCTTAGAGGGAGGATGGGAAGGAATGATTGCAGGTTGG | |
| | CACGGATACACATCCCATGGGGCACATGGAGTAGCGGTGGCAGCTG | |
| | ACCTTAAGAGCACTCAAGAGGCCATAAACAAGATAACAAAAAATCTCA | |
| | ACTCTTTGAGTGAGCTGGAAGTAAAGAATCTTCAAAGACTAAGCGGT | |
| | GCCATGGATGAACTCCACAACGAAATACTAGAACTAGATGAGAAAGT | |
| | GGATGATCTCAGAGCTGACACAATAAGCTCACAAATAGAACTCGCAG | |
| | TCCTGCTTTCCAATGAAGGAATAATAAACAGTGAAGATGAACATCTCT | |
| | TGGCGCTTGAAAGAAAGCTGAAGAAAATGCTGGGCCCCTCTGCTGTA | |
| | GAGATAGGGAATGGATGCTTTGAAACCAAACACAAGTGCAACCAGAC | |
| | CTGCCTCGACAGAATAGCTGCTGGTACCTTTGATGCAGGAGAATTTT | |
| | CTCTCCCCACCTTTGATTCACTGAATATTACTGCTGCATCTTTAAATG | |
| | ACGACGGATTGGACAATCATACTATACTGCTTTACTACTCAACTGCTG | |
| | CCTCCAGTTTGGCTGTAACACTGATGATAGCTATCTTTGTTGTTTATA | |
| | TGGTCTCCAGAGACAATGTTTCTTGCTCCATTTGTCTATAAGGAAAGT | |
| | TAAGCCCTGTATTTTCCTTTATTGTAGTGCTTGTTTGCTTGTTGTCATT | |
| | ACAAAGAAACGTTATTGAAAAATGGAAGCGGAGCCACGAACTTCTCT | |
| | CTGTTAAAGCAAGCAGGAGATGTTGAAGAAAACCCCGGGCCTTTCGG | |
| | AGCTATTGCTGGTTTCTTGGAAGGAGGATGGGAAGGAATGATTGCAG | |
| | GTTGGCACGGATACACATCTCATGGAGCACATGGAGTAGCAGTGGC | |
| | AGCAGACCTTAAGAGTACCCATGATGA | |
| Pan-CoV-Flu | ATGTTCGTGTTCCTGGTGCTGCTGCCCCTGGTGAGCAGCCAGTGCG | 415 |
| Vaccine #27 | TGAACCTGATCACCAGGACCCAGAGCTACACCAACAGCTTCACCAGG | |
| CoV- Omicron | GGCGTGTACTACCCCGACAAGGTGTTCAGGAGCAGCGTGCTGCACA | |
| BA.2 | GCACCCAGGACCTGTTCCTGCCCTTCTTCAGCAACGTGACCTGGTTC | |
| Spike(HexaPra)- | CACGCCATCCACGTGAGCGGCACCAACGGCACCAAGAGGTTCGACA | |
| Influenza (HA) | ACCCCGTGCTGCCCTTCAACGACGGCGTGTACTTCGCCAGCACCGA | |
| | GAAGAGCAACATCATCAGGGGCTGGATCTTCGGCACCACCCTGGAC | |
| | AGCAAGACCCAGAGCCTGCTGATCGTGAACAACGCCACCAACGTGG | |
| | TGATCAAGGTGTGCGAGTTCCAGTTCTGCAACGACCCCTTCCTGGAC | |
| | GTGTACTACCACAAGAACAACAAGAGCTGGATGGAGAGCGAGTTCA | |
| | GGGTGTACAGCAGCGCCAACAACTGCACCTTCGAGTACGTGAGCCA | |
| | GCCCTTCCTGATGGACCTGGAGGGCAAGCAGGGCAACTTCAAGAAC | |
| | CTGAGGGAGTTCGTGTTCAAGAACATCGACGGCTACTTCAAGATCTA | |
| | CAGCAAGCACACCCCCATCAACCTGGGCAGGGACCTGCCCCAGGGC | |
| | TTCAGCGCCCTGGAGCCCCTGGTGGACCTGCCCATCGGCATCAACA | |
| | TCACCAGGTTCCAGACCCTGCTGGCCCTGCACAGGAGCTACCTGAC | |
| | CCCCGGCGACAGCAGCAGCGGCTGGACCGCCGGCGCCGCCGCCTA | |
| | CTACGTGGGCTACCTGCAGCCCAGGACCTTCCTGCTGAAGTACAAC | |
| | GAGAACGGCACCATCACCGACGCCGTGGACTGCGCCCTGGACCCC | |
| | CTGAGCGAGACCAAGTGCACCCTGAAGAGCTTCACCGTGGAGAAGG | |
| | GCATCTACCAGACCAGCAACTTCAGGGTGCAGCCCACCGAGAGCAT | |
| | CGTGAGGTTCCCCAACATCACCAACCTGTGCCCCTTCGACGAGGTGT | |
| | TCAACGCCACCAGGTTCGCCAGCGTGTACGCCTGGAACAGGAAGAG | |
| | GATCAGCAACTGCGTGGCCGACTACAGCGTGCTGTACAACTTCGCC | |
| | CCCTTCTTCGCCTTCAAGTGCTACGGCGTGAGCCCCACCAAGCTGAA | |
| | CGACCTGTGCTTCACCAACGTGTACGCCGACAGCTTCGTGATCAGG | |
| | GGCAACGAGGTGAGCCAGATCGCCCCCGGCCAGACCGGCAACATC | |
| | GCCGACTACAACTACAAGCTGCCCGACGACTTCACCGGCTGCGTGA | |
| | TCGCCTGGAACAGCAACAAGCTGGACAGCAAGGTGGGCGGCAACTA | |
| | CAACTACCTGTACAGGCTGTTCAGGAAGAGCAACCTGAAGCCCTTCG | |
| | AGAGGGACATCAGCACCGAGATCTACCAGGCCGGCAACAAGCCCTG | |
| | CAACGGCGTGGCCGGCTTCAACTGCTACTTCCCCCTGAGGAGCTAC | |
| | GGCTTCAGGCCCACCTACGGCGTGGGCCACCAGCCCTACAGGGTG | |
| | GTGGTGCTGAGCTTCGAGCTGCTGCACGCCCCCGCCACCGTGTGCG | |
| | GCCCCAAGAAGAGCACCAACCTGGTGAAGAACAAGTGCGTGAACTT | |
| | CAACTTCAACGGCCTGACCGGCACCGGCGTGCTGACCGAGAGCAAC | |
| | AAGAAGTTCCTGCCCTTCCAGCAGTTCGGCAGGGACATCGCCGACA | |
| | CCACCGACGCCGTGAGGGACCCCCAGACCCTGGAGATCCTGGACAT | |
| | CACCCCCTGCAGCTTCGGCGGCGTGAGCGTGATCACCCCCGGCACC | |
| | AACACCAGCAACCAGGTGGCCGTGCTGTACCAGGGCGTGAACTGCA | |
| | CCGAGGTGCCCGTGGCCATCCACGCCGACCAGCTGACCCCCACCTG | |
| | GAGGGTGTACAGCACCGGCAGCAACGTGTTCCAGACCAGGGCCGG | |
| | CTGCCTGATCGGCGCCGAGTACGTGAACAACAGCTACGAGTGCGAC | |
| | ATCCCCATCGGCGCCGGCATCTGCGCCAGCTACCAGACCCAGACCA | |
| | AGAGCCACCAGCAGGCCCAGAGCGTGGCCAGCCAGAGCATCATCG | |
| | CCTACACCATGAGCCTGGGCGCCGAGAACAGCGTGGCCTACAGCAA | |
| | CAACAGCATCGCCATCCCCACCAACTTCACCATCAGCGTGACCACCG | |
| | AGATCCTGCCCGTGAGCATGACCAAGACCAGCGTGGACTGCACCAT | |
| | GTACATCTGCGGCGACAGCACCGAGTGCAGCAACCTGCTGCTGCAG | |
| | TACGGCAGCTTCTGCACCCAGCTGAAGAGGGCCCTGACCGGCATCG | |
| | CCGTGGAGCAGGACAAGAACACCCAGGAGGTGTTCGCCCAGGTGAA | |
| | GCAGATCTACAAGACCCCCCCCATCAAGTACTTCGGCGGCTTCAACT | |
| | TCAGCCAGATCCTGCCCGACCCCAGCAAGCCCAGCAAGAGGAGCCC | |
| | CATCGAGGACCTGCTGTTCAACAAGGTGACCCTGGCCGACGCCGGC | |
| | TTCATCAAGCAGTACGGCGACTGCCTGGGCGACATCGCCGCCAGGG | |
| | ACCTGATCTGCGCCCAGAAGTTCAACGGCCTGACCGTGCTGCCCCC | |
| | CCTGCTGACCGACGAGATGATCGCCCAGTACACCAGCGCCCTGCTG | |
| | GCCGGCACCATCACCAGCGGCTGGACCTTCGGCGCCGGCCCCGCC | |
| | CTGCAGATCCCCTTCCCCATGCAGATGGCCTACAGGTTCAACGGCAT | |
| | CGGCGTGACCCAGAACGTGCTGTACGAGAACCAGAAGCTGATCGCC | |
| | AACCAGTTCAACAGCGCCATCGGCAAGATCCAGGACAGCCTGAGCA | |
| | GCACCCCCAGCGCCCTGGGCAAGCTGCAGGACGTGGTGAACCACAA | |
| | CGCCCAGGCCCTGAACACCCTGGTGAAGCAGCTGAGCAGCAAGTTC | |
| | GGCGCCATCAGCAGCGTGCTGAACGACATCCTGAGCAGGCTGGACC | |
| | CCCCCGAGGCCGAGGTGCAGATCGACAGGCTGATCACCGGCAGGC | |
| | TGCAGAGCCTGCAGACCTACGTGACCCAGCAGCTGATCAGGGCCGC | |
| | CGAGATCAGGGCCAGCGCCAACCTGGCCGCCACCAAGATGAGCGA | |
| | GTGCGTGCTGGGCCAGAGCAAGAGGGTGGACTTCTGCGGCAAGGG | |
| | CTACCACCTGATGAGCTTCCCCCAGAGCGCCCCCCACGGCGTGGTG | |
| | TTCCTGCACGTGACCTACGTGCCCGCCCAGGAGAAGAACTTCACCA | |
| | CCGCCCCCGCCATCTGCCACGACGGCAAGGCCCACTTCCCCAGGGA | |
| | GGGCGTGTTCGTGAGCAACGGCACCCACTGGTTCGTGACCCAGAGG | |
| | AACTTCTACGAGCCCCAGATCATCACCACCGACAACACCTTCGTGAG | |
| | CGGCAACTGCGACGTGGTGATCGGCATCGTGAACAACACCGTGTAC | |
| | GACCCCCTGCAGCCCGAGCTGGACAGCTTCAAGGAGGAGCTGGACA | |
| | AGTACTTCAAGAACCACACCAGCCCCGACGTGGACCTGGGCGACAT | |
| | CAGCGGCATCAACGCCAGCGTGGTGAACATCCAGAAGGAGATCGAC | |
| | AGGCTGAACGAGGTGGCCAAGAACCTGAACGAGAGCCTGATCGACC | |
| | TGCAGGAGCTGGGCAAGTACGAGCAGTACATCAAGTGGCCCTGGTA | |
| | CATCTGGCTGGGCTTCATCGCCGGCCTGATCGCCATCGTGATGGTG | |
| | ACCATCATGCTGTGCTGCATGACCAGCTGCTGCAGCTGCCTGAAGG | |
| | GCTGCTGCAGCTGCGGCAGCTGCTGCAAGTTCGACGAGGACGACAG | |
| | CGAGCCCGTGCTGAAGGGCGTGAAGCTGCACTACACCGGAAGCGG | |
| | AGCCACGAACTTCTCTCTGTTAAAGCAAGCAGGAGATGTTGAAGAAA | |
| | ACCCCGGGCCTATGAAGGCAATACTAGTAGTTCTGCTATATACATTTG | |
| | CAACCGCAAATGCAGACACATTATGTATAGGTTATCATGCGAACAACT | |
| | CAACAGACACTGTAGACACAGTACTAGAAAAGAATGTAACAGTAACA | |
| | CACTCTGTTAACCTTCTAGAAGACAAGCATAACGGGAAACTATGCAAA | |
| | CTAAGAGGGGTAGCCCCATTGCATTTGGGTAAATGTAACATTGCTGG | |
| | CTGGATCCTGGGAAATCCAGAGTGTGAATCACTCTCCACAGCAAGCT | |
| | CATGGTCCTACATTGTGGAAACATCTAGTTCAGACAATGGAACGTGTT | |
| | ACCCAGGAGATTTCATCGATTATGAGGAGCTAAGAGAGCAATTGAGC | |
| | TCAGTGTCATCATTTGAAAGGTTTGAGATATTCCCCAAGACAAGTTCA | |
| | TGGCCCAATCATGACTCGAACAAAGGTGTAACGACAGCATGTCCTCA | |
| | TGCTGGAGCAAAAAGCTTCTACAAAAATTTAATATGGCTAGTTAAAAA | |
| | AGGAAATTCATACCCAAAGCTCAGCAAATCCTACATTAATGATAAAGG | |
| | GAAAGAAGTCCTCGTGCTATGGGGCATTCACCATCCACCTACTAGTG | |
| | CTGACCAACAAAGTCTCTATCAGAATGCAGATGCATATGTTTTTGTGG | |
| | GGACATCAAGATACAGCAAGAAGTTCAAGCCGGAAATAGCAATAAGG | |
| | CCCAAAGTGAGGGATCAAGAAGGGAGAATGAACTATTACTGGACACT | |
| | AGTAGAGCCGGGAGACAAAATAACATTCGAAGCAACTGGAAATCTAG | |
| | TGGTACCGAGATATGCATTCGCAATGGAAAGAAATGCTGGATCTGGT | |
| | ATTATCATTTCAGATACACCAGTCCACGATTGCAATACAACTTGTCAG | |
| | ACACCCAAGGGTGCTATAAACACCAGCCTCCCATTTCAGAACATACA | |
| | TCCGATCACAATTGGAAAATGTCCAAAATATGTAAAAAGCACAAAATT | |
| | GAGACTGGCCACAGGATTGAGGAATGTCCCGTCCATTCAATCTAGAG | |
| | GCCTATTTGGGGCCATTGCCGGTTTCATTGAAGGGGGGTGGACAGG | |
| | GATGGTAGATGGATGGTACGGTTATCACCATCAAAATGAGCAGGGGT | |
| | CAGGATATGCAGCCGACCTGAAGAGCACACAGAATGCCATTGACGA | |
| | GATTACTAACAAAGTAAACTCTGTTATTGAAAAAATGAATACACAGTTC | |
| | ACAGCAGTAGGTAAAGAGTTCAACCACCTGGAAAAAAGAATAGAGAA | |
| | TTTAAATAAAAAAGTTGATGATGGTTTCCTGGACATTTGGACTTACAAT | |
| | GCCGAACTGTTGGTTCTATTGGAAAATGAAAGAACTTTGGACTACCA | |
| | CGATTCAAAGGTGAAGAACTTATATGAAAAGGTAAGAAGCCAGTTAAA | |
| | AAACAATGCCAAGGAAATTGGAAACGGCTGCTTTGAATTTTACCACAA | |
| | ATGTGATAACACGTGCATGGAAAGTGTCAAAAATGGGACTTATGACT | |
| | ACCCAAAATACTGAGAGGAAGGAAAATTAAACAGAGAAGAAATAGAT | |
| | GGGGTAAAGCTGGAATCAACAAGGATTTACCAGATTTTGGCGATCTA | |
| | TTCAACCGTCGCCAGTTCATTGGTACTGGTAGTCTCCCTGGGGGCAA | |
| | TCAGTTTCTGGATGTGCTCTAATGGGTCTCTACAGTGTAGAATATGTA | |
| | TTTAAGGAAGCGGAGCCACGAACTTCTCTCTGTTAAAGCAAGCAGGA | |
| | GATGTTGAAGAAAACCCCGGGCCTAGCAAAAGCAGGGGATAATTCTA | |
| | TTAACCATGAAGACTATCATTGCTTTGAGCTACATTCTATGTCTGGTTT | |
| | TCGCTCAAAAACTTCCTGGAAATGACAATAGCACTGCAACGCTGTGC | |
| | CTTGGGCACCATGCAGTACCAAACGGAACGATAGTGAAAACAATCAC | |
| | GAATGACCAAATTGAAGTTACTAATGCTACTGAGCTGGTTCAGAATTC | |
| | CTCAATAGGTGAAATATGCGACAGTCCTCATCAGATCCTTGATGGAG | |
| | AAAACTGCACACTAATAGATGCTCTATTGGGAGACCCTCAGTGTGAT | |
| | GGCTTTCAAAATAAGAAATGGGACCTTTTTGTTGAACGAAGCAAAGC | |
| | CTACAGCAACTGTTACCCTTATGATGTGCCGGATTATGCCTCCCTTAG | |
| | GTCACTAGTTGCCTCATCCGGCACACTGGAGTTTAACAATGAAAGCT | |
| | TCAATTGGGCTGGAGTCACTCAAAACGGAACAAGTTCTGCTTGCATA | |
| | AGGGGATCTAATAGTAGTTTCTTTAGTAGATTAAATTGGTTGACCCAC | |
| | TTAAACTTCAAGTACCCAGCATTGAACGTGACTATGCCAAACAATGAA | |
| | CAATTTGACAAATTGTACATTTGGGGGGTTCACCACCCGGGTACGGA | |
| | CAAGGACCAAATCTTCCTGTATGCTCAATCATCAGGAAGAATCACAGT | |
| | ATCTACCAAAAGAAGCCAACAAGCTGTAATCCCGAATATCGGATCTA | |
| | GACCCAGAATAAGGAATATCCCTAGCAGAATAAGCATCTATTGGACA | |
| | ATAGTAAAACCGGGAGACATACTTTTGATTAACAGCACAGGGAATCTA | |
| | ATTGCTCCTAGGGGTTACTTCAAAATACGAAGTGGGAAAAGCTCAAT | |
| | AATGAGATCAGATGCACCCATTGGCAAATGCAAGTCTGAATGCATCA | |
| | CTCCAAATGGAAGCATTCCCAATGACAAACCATTCCAAAATGTAAACA | |
| | GGATCACATACGGGGCCTGTCCCAGATATGTTAAGCAAAGCACTCTG | |
| | AAATTGGCAACAGGAATGCGAAATGTACCAGAGAAACAAACTAGAGG | |
| | CATATTTGGCGCAATAGCGGGTTTCATAGAAAATGGTTGGGAGGGAA | |
| | TGGTGGATGGTTGGTACGGTTTCAGGCATCAAAATTCTGAGGGAAGA | |
| | GGACAAGCAGCAGATCTCAAAAGCACTCAAGCAGCAATCGATCAAAT | |
| | CAATGGGAAGCTGAATCGATTGATCGGGAAAACCAACGAGAAATTCC | |
| | ATCAGATTGAGAAAGAATTCTCAGAAGTAGAAGGGAGAATTCAGGAC | |
| | CTTGAGAAATATGTTGAGGACACAAAAATAGATCTCTGGTCATACAAC | |
| | GCAGAGCTTCTTGTTGCCCTGGAAAACCAACATACAATTGATCTAACT | |
| | GACTCAGAAATGAACAAACTGTTTGAAAAAACAAAGAAGCAACTGAG | |
| | GGAAAATGCTGAGGATATGGGCAATGGTTGTTTCAAAATATACCACA | |
| | AATGTGACAATGCCTGCATAGGATCAATCAGAAATGGAACTTATGAC | |
| | CACGATGTATACAGGGATGAAGCATTAAACAACCGGTTCCAGATCAA | |
| | GGGAGTTGAGCTGAAGTCAGGGTACAAAGATTGGATCCTATGGATTT | |
| | CCTTTGCCATATCATGTTTTTTGCTTTGTGTTGCTTTGTTGGGGTTCAT | |
| | CATGTGGGCCTGCCAAAAGGGCAACATTAGGTGCAACATTTGCATTT | |
| | GAGTGCATTAATTAAAAACACCCTTGTTTCTACTGGAAGCGGAGCCA | |
| | CGAACTTCTCTCTGTTAAAGCAAGCAGGAGATGTTGAAGAAAACCCC | |
| | GGGCCTATTTTCTAATATCCACAAAATGAAGGCAATAATTGTACTACT | |
| | CATGGTAGTAACATCCAATGCAGATCGAATCTGCACTGGGATAACAT | |
| | CGTCAAACTCACCACATGTCGTCAAAACTGCTACTCAAGGGGAGGTC | |
| | AACGTGACCGGTGTAATACCACTGACAACAACACCCACCAAATCTCA | |
| | TTTTGCAAATCTCAAAGGAACAGAAACCAGGGGGAAACTATGCCCAA | |
| | AATGCCTCAACTGCACAGATCTGGATGTAGCCTTGGGCAGACCAAAA | |
| | TGCACAGGGAAAATACCCTCTGCAAGGGTTTCAATACTCCATGAAGT | |
| | CAGACCTGTTACATCTGGGTGCTTTCCTATAATGCACGATAGAACAAA | |
| | AATTAGACAGCTGCCTAACCTTCTCCGAGGATACGAACATGTCAGGT | |
| | TATCAACTCACAACGTTATCAATGCAAAAGATGCACCAGGAAGACCC | |
| | TACGAAATTGGAACCTCAGGGTCTTGCCCTAACATTACCAATGGAAA | |
| | CGGATTCTTCGCAACAATGGCTTGGGCCGTCCCAAAAAACAAAACAG | |
| | CAACAAATCCATTAACAATAGAAGTACCATACATTTGTACAGAAGGAG | |
| | AAGACCAAATTACCGTTTGGGGGTTCCACTCTGACAACGAGACCCAA | |
| | ATGGCAAAGCTCTATGGGGACTCAAAGCCCCAGAAGTTCACCTCATC | |
| | TGCCAACGGAGTGACCACACATTACGTTTCACAGATTGGTGGCTTCC | |
| | CAAATCAAACAGAAGACGGAGGACTACCACAAAGTGGCAGAATTGTT | |
| | GTTGATTACATGGTGCAGAAATCTGGAAAAACAGGAACAATTACCTAT | |
| | CAAAGAGGTATTTTATTGCCTCAAAAAGTGTGGTGCGCAAGTGGCAG | |
| | GAGCAAGGTAATAAAAGGATCCTTGCCCTTAATTGGAGAAGCAGATT | |
| | GCCTCCATGAAAAATACGGTGGATTAAACAAAAGCAAGCCTTACTAC | |
| | ACAGGGGAACATGCAAAGGCCATAGGAAATTGCCCAATATGGGTGAA | |
| | AACACCCTTGAAGCTGGCCAATGGAACCAAATATAGACCCCCTGCAA | |
| | AACTATTAAAGGAAAGAGGTTTCTTCGGAGCCATTGCTGGTTTCTTAG | |
| | AGGGAGGATGGGAAGGAATGATTGCAGGTTGGCACGGATACACATC | |
| | CCATGGGGCACATGGAGTAGCGGTGGCAGCTGACCTTAAGAGCACT | |
| | CAAGAGGCCATAAACAAGATAACAAAAAATCTCAACTCTTTGAGTGAG | |
| | CTGGAAGTAAAGAATCTTCAAAGACTAAGCGGTGCCATGGATGAACT | |
| | CCACAACGAAATACTAGAACTAGATGAGAAAGTGGATGATCTCAGAG | |
| | CTGACACAATAAGCTCACAAATAGAACTCGCAGTCCTGCTTTCCAATG | |
| | AAGGAATAATAAACAGTGAAGATGAACATCTCTTGGCGCTTGAAAGA | |
| | AAGCTGAAGAAAATGCTGGGCCCCTCTGCTGTAGAGATAGGGAATG | |
| | GATGCTTTGAAACCAAACACAAGTGCAACCAGACCTGCCTCGACAGA | |
| | ATAGCTGCTGGTACCTTTGATGCAGGAGAATTTTCTCTCCCCACCTTT | |
| | GATTCACTGAATATTACTGCTGCATCTTTAAATGACGACGGATTGGAC | |
| | AATCATACTATACTGCTTTACTACTCAACTGCTGCCTCCAGTTTGGCT | |
| | GTAACACTGATGATAGCTATCTTTGTTGTTTATATGGTCTCCAGAGAC | |
| | AATGTTTCTTGCTCCATTTGTCTATAAGGAAAGTTAAGCCCTGTATTTT | |
| | CCTTTATTGTAGTGCTTGTTTGCTTGTTGTCATTACAAAGAAACGTTAT | |
| | TGAAAAATTGATGA | |
| Pan-CoV-Flu | ATGTTCGTGTTCCTGGTGCTGCTGCCCCTGGTGAGCAGCCAGTGCG | 416 |
| Vaccine #28 | TGAACCTGATCACCAGGACCCAGAGCTACACCAACAGCTTCACCAGG | |
| CoV-Omicron | GGCGTGTACTACCCCGACAAGGTGTTCAGGAGCAGCGTGCTGCACA | |
| BA.2 Spike(2 | GCACCCAGGACCTGTTCCTGCCCTTCTTCAGCAACGTGACCTGGTTC | |
| proline | CACGCCATCCACGTGAGCGGCACCAACGGCACCAAGAGGTTCGACA | |
| substitution)- | ACCCCGTGCTGCCCTTCAACGACGGCGTGTACTTCGCCAGCACCGA | |
| Influenza (HA) | GAAGAGCAACATCATCAGGGGCTGGATCTTCGGCACCACCCTGGAC | |
| | AGCAAGACCCAGAGCCTGCTGATCGTGAACAACGCCACCAACGTGG | |
| | TGATCAAGGTGTGCGAGTTCCAGTTCTGCAACGACCCCTTCCTGGAC | |
| | GTGTACTACCACAAGAACAACAAGAGCTGGATGGAGAGCGAGTTCA | |
| | GGGTGTACAGCAGCGCCAACAACTGCACCTTCGAGTACGTGAGCCA | |
| | GCCCTTCCTGATGGACCTGGAGGGCAAGCAGGGCAACTTCAAGAAC | |
| | CTGAGGGAGTTCGTGTTCAAGAACATCGACGGCTACTTCAAGATCTA | |
| | CAGCAAGCACACCCCCATCAACCTGGGCAGGGACCTGCCCCAGGGC | |
| | TTCAGCGCCCTGGAGCCCCTGGTGGACCTGCCCATCGGCATCAACA | |
| | TCACCAGGTTCCAGACCCTGCTGGCCCTGCACAGGAGCTACCTGAC | |
| | CCCCGGCGACAGCAGCAGCGGCTGGACCGCCGGCGCCGCCGCCTA | |
| | CTACGTGGGCTACCTGCAGCCCAGGACCTTCCTGCTGAAGTACAAC | |
| | GAGAACGGCACCATCACCGACGCCGTGGACTGCGCCCTGGACCCC | |
| | CTGAGCGAGACCAAGTGCACCCTGAAGAGCTTCACCGTGGAGAAGG | |
| | GCATCTACCAGACCAGCAACTTCAGGGTGCAGCCCACCGAGAGCAT | |
| | CGTGAGGTTCCCCAACATCACCAACCTGTGCCCCTTCGACGAGGTGT | |
| | TCAACGCCACCAGGTTCGCCAGCGTGTACGCCTGGAACAGGAAGAG | |
| | GATCAGCAACTGCGTGGCCGACTACAGCGTGCTGTACAACTTCGCC | |
| | CCCTTCTTCGCCTTCAAGTGCTACGGCGTGAGCCCCACCAAGCTGAA | |
| | CGACCTGTGCTTCACCAACGTGTACGCCGACAGCTTCGTGATCAGG | |
| | GGCAACGAGGTGAGCCAGATCGCCCCCGGCCAGACCGGCAACATC | |
| | GCCGACTACAACTACAAGCTGCCCGACGACTTCACCGGCTGCGTGA | |
| | TCGCCTGGAACAGCAACAAGCTGGACAGCAAGGTGGGCGGCAACTA | |
| | CAACTACCTGTACAGGCTGTTCAGGAAGAGCAACCTGAAGCCCTTCG | |
| | AGAGGGACATCAGCACCGAGATCTACCAGGCCGGCAACAAGCCCTG | |
| | CAACGGCGTGGCCGGCTTCAACTGCTACTTCCCCCTGAGGAGCTAC | |
| | GGCTTCAGGCCCACCTACGGCGTGGGCCACCAGCCCTACAGGGTG | |
| | GTGGTGCTGAGCTTCGAGCTGCTGCACGCCCCCGCCACCGTGTGCG | |
| | GCCCCAAGAAGAGCACCAACCTGGTGAAGAACAAGTGCGTGAACTT | |
| | CAACTTCAACGGCCTGACCGGCACCGGCGTGCTGACCGAGAGCAAC | |
| | AAGAAGTTCCTGCCCTTCCAGCAGTTCGGCAGGGACATCGCCGACA | |
| | CCACCGACGCCGTGAGGGACCCCCAGACCCTGGAGATCCTGGACAT | |
| | CACCCCCTGCAGCTTCGGCGGCGTGAGCGTGATCACCCCCGGCACC | |
| | AACACCAGCAACCAGGTGGCCGTGCTGTACCAGGGCGTGAACTGCA | |
| | CCGAGGTGCCCGTGGCCATCCACGCCGACCAGCTGACCCCCACCTG | |
| | GAGGGTGTACAGCACCGGCAGCAACGTGTTCCAGACCAGGGCCGG | |
| | CTGCCTGATCGGCGCCGAGTACGTGAACAACAGCTACGAGTGCGAC | |
| | ATCCCCATCGGCGCCGGCATCTGCGCCAGCTACCAGACCCAGACCA | |
| | AGAGCCACCAGCAGGCCCAGAGCGTGGCCAGCCAGAGCATCATCG | |
| | CCTACACCATGAGCCTGGGCGCCGAGAACAGCGTGGCCTACAGCAA | |
| | CAACAGCATCGCCATCCCCACCAACTTCACCATCAGCGTGACCACCG | |
| | AGATCCTGCCCGTGAGCATGACCAAGACCAGCGTGGACTGCACCAT | |
| | GTACATCTGCGGCGACAGCACCGAGTGCAGCAACCTGCTGCTGCAG | |
| | TACGGCAGCTTCTGCACCCAGCTGAAGAGGGCCCTGACCGGCATCG | |
| | CCGTGGAGCAGGACAAGAACACCCAGGAGGTGTTCGCCCAGGTGAA | |
| | GCAGATCTACAAGACCCCCCCCATCAAGTACTTCGGCGGCTTCAACT | |
| | TCAGCCAGATCCTGCCCGACCCCAGCAAGCCCAGCAAGAGGAGCTT | |
| | CATCGAGGACCTGCTGTTCAACAAGGTGACCCTGGCCGACGCCGGC | |
| | TTCATCAAGCAGTACGGCGACTGCCTGGGCGACATCGCCGCCAGGG | |
| | ACCTGATCTGCGCCCAGAAGTTCAACGGCCTGACCGTGCTGCCCCC | |
| | CCTGCTGACCGACGAGATGATCGCCCAGTACACCAGCGCCCTGCTG | |
| | GCCGGCACCATCACCAGCGGCTGGACCTTCGGCGCCGGCGCCGCC | |
| | CTGCAGATCCCCTTCGCCATGCAGATGGCCTACAGGTTCAACGGCAT | |
| | CGGCGTGACCCAGAACGTGCTGTACGAGAACCAGAAGCTGATCGCC | |
| | AACCAGTTCAACAGCGCCATCGGCAAGATCCAGGACAGCCTGAGCA | |
| | GCACCGCCAGCGCCCTGGGCAAGCTGCAGGACGTGGTGAACCACA | |
| | ACGCCCAGGCCCTGAACACCCTGGTGAAGCAGCTGAGCAGCAAGTT | |
| | CGGCGCCATCAGCAGCGTGCTGAACGACATCCTGAGCAGGCTGGAC | |
| | CCCCCCGAGGCCGAGGTGCAGATCGACAGGCTGATCACCGGCAGG | |
| | CTGCAGAGCCTGCAGACCTACGTGACCCAGCAGCTGATCAGGGCCG | |
| | CCGAGATCAGGGCCAGCGCCAACCTGGCCGCCACCAAGATGAGCG | |
| | AGTGCGTGCTGGGCCAGAGCAAGAGGGTGGACTTCTGCGGCAAGG | |
| | GCTACCACCTGATGAGCTTCCCCCAGAGCGCCCCCCACGGCGTGGT | |
| | GTTCCTGCACGTGACCTACGTGCCCGCCCAGGAGAAGAACTTCACC | |
| | ACCGCCCCCGCCATCTGCCACGACGGCAAGGCCCACTTCCCCAGGG | |
| | AGGGCGTGTTCGTGAGCAACGGCACCCACTGGTTCGTGACCCAGAG | |
| | GAACTTCTACGAGCCCCAGATCATCACCACCGACAACACCTTCGTGA | |
| | GCGGCAACTGCGACGTGGTGATCGGCATCGTGAACAACACCGTGTA | |
| | CGACCCCCTGCAGCCCGAGCTGGACAGCTTCAAGGAGGAGCTGGAC | |
| | AAGTACTTCAAGAACCACACCAGCCCCGACGTGGACCTGGGCGACA | |
| | TCAGCGGCATCAACGCCAGCGTGGTGAACATCCAGAAGGAGATCGA | |
| | CAGGCTGAACGAGGTGGCCAAGAACCTGAACGAGAGCCTGATCGAC | |
| | CTGCAGGAGCTGGGCAAGTACGAGCAGTACATCAAGTGGCCCTGGT | |
| | ACATCTGGCTGGGCTTCATCGCCGGCCTGATCGCCATCGTGATGGT | |
| | GACCATCATGCTGTGCTGCATGACCAGCTGCTGCAGCTGCCTGAAG | |
| | GGCTGCTGCAGCTGCGGCAGCTGCTGCAAGTTCGACGAGGACGACA | |
| | GCGAGCCCGTGCTGAAGGGCGTGAAGCTGCACTACACCGGAAGCG | |
| | GAGCCACGAACTTCTCTCTGTTAAAGCAAGCAGGAGATGTTGAAGAA | |
| | AACCCCGGGCCTATGAAGGCAATACTAGTAGTTCTGCTATATACATTT | |
| | GCAACCGCAAATGCAGACACATTATGTATAGGTTATCATGCGAACAA | |
| | CTCAACAGACACTGTAGACACAGTACTAGAAAAGAATGTAACAGTAA | |
| | CACACTCTGTTAACCTTCTAGAAGACAAGCATAACGGGAAACTATGC | |
| | AAACTAAGAGGGGTAGCCCCATTGCATTTGGGTAAATGTAACATTGC | |
| | TGGCTGGATCCTGGGAAATCCAGAGTGTGAATCACTCTCCACAGCAA | |
| | GCTCATGGTCCTACATTGTGGAAACATCTAGTTCAGACAATGGAACG | |
| | TGTTACCCAGGAGATTTCATCGATTATGAGGAGCTAAGAGAGCAATT | |
| | GAGCTCAGTGTCATCATTTGAAAGGTTTGAGATATTCCCCAAGACAA | |
| | GTTCATGGCCCAATCATGACTCGAACAAAGGTGTAACGACAGCATGT | |
| | CCTCATGCTGGAGCAAAAAGCTTCTACAAAAATTTAATATGGCTAGTT | |
| | AAAAAAGGAAATTCATACCCAAAGCTCAGCAAATCCTACATTAATGAT | |
| | AAAGGGAAAGAAGTCCTCGTGCTATGGGGCATTCACCATCCACCTAC | |
| | TAGTGCTGACCAACAAAGTCTCTATCAGAATGCAGATGCATATGTTTT | |
| | TGTGGGGACATCAAGATACAGCAAGAAGTTCAAGCCGGAAATAGCAA | |
| | TAAGGCCCAAAGTGAGGGATCAAGAAGGGAGAATGAACTATTACTGG | |
| | ACACTAGTAGAGCCGGGAGACAAAATAACATTCGAAGCAACTGGAAA | |
| | TCTAGTGGTACCGAGATATGCATTCGCAATGGAAAGAAATGCTGGAT | |
| | CTGGTATTATCATTTCAGATACACCAGTCCACGATTGCAATACAACTT | |
| | GTCAGACACCCAAGGGTGCTATAAACACCAGCCTCCCATTTCAGAAC | |
| | ATACATCCGATCACAATTGGAAAATGTCCAAAATATGTAAAAAGCACA | |
| | AAATTGAGACTGGCCACAGGATTGAGGAATGTCCCGTCCATTCAATC | |
| | TAGAGGCCTATTTGGGGCCATTGCCGGTTTCATTGAAGGGGGGTGG | |
| | ACAGGGATGGTAGATGGATGGTACGGTTATCACCATCAAAATGAGCA | |
| | GGGGTCAGGATATGCAGCCGACCTGAAGAGCACACAGAATGCCATT | |
| | GACGAGATTACTAACAAAGTAAACTCTGTTATTGAAAAAATGAATACA | |
| | CAGTTCACAGCAGTAGGTAAAGAGTTCAACCACCTGGAAAAAAGAAT | |
| | AGAGAATTTAAATAAAAAAGTTGATGATGGTTTCCTGGACATTTGGAC | |
| | TTACAATGCCGAACTGTTGGTTCTATTGGAAAATGAAAGAACTTTGGA | |
| | CTACCACGATTCAAAGGTGAAGAACTTATATGAAAAGGTAAGAAGCC | |
| | AGTTAAAAAACAATGCCAAGGAAATTGGAAACGGCTGCTTTGAATTTT | |
| | ACCACAAATGTGATAACACGTGCATGGAAAGTGTCAAAAATGGGACT | |
| | TATGACTACCCAAAATACTCAGAGGAAGCAAAATTAAACAGAGAAGAA | |
| | ATAGATGGGGTAAAGCTGGAATCAACAAGGATTTACCAGATTTTGGC | |
| | GATCTATTCAACCGTCGCCAGTTCATTGGTACTGGTAGTCTCCCTGG | |
| | GGGCAATCAGTTTCTGGATGTGCTCTAATGGGTCTCTACAGTGTAGA | |
| | ATATGTATTTAAGGAAGCGGAGCCACGAACTTCTCTCTGTTAAAGCAA | |
| | GCAGGAGATGTTGAAGAAAACCCCGGGCCTAGCAAAAGCAGGGGAT | |
| | AATTCTATTAACCATGAAGACTATCATTGCTTTGAGCTACATTCTATGT | |
| | CTGGTTTTCGCTCAAAAACTTCCTGGAAATGACAATAGCACTGCAAC | |
| | GCTGTGCCTTGGGCACCATGCAGTACCAAACGGAACGATAGTGAAAA | |
| | CAATCACGAATGACCAAATTGAAGTTACTAATGCTACTGAGCTGGTTC | |
| | AGAATTCCTCAATAGGTGAAATATGCGACAGTCCTCATCAGATCCTTG | |
| | ATGGAGAAAACTGCACACTAATAGATGCTCTATTGGGAGACCCTCAG | |
| | TGTGATGGCTTTCAAAATAAGAAATGGGACCTTTTTGTTGAACGAAGC | |
| | AAAGCCTACAGCAACTGTTACCCTTATGATGTGCCGGATTATGCCTC | |
| | CCTTAGGTCACTAGTTGCCTCATCCGGCACACTGGAGTTTAACAATG | |
| | AAAGCTTCAATTGGGCTGGAGTCACTCAAAACGGAACAAGTTCTGCT | |
| | TGCATAAGGGGATCTAATAGTAGTTTCTTTAGTAGATTAAATTGGTTG | |
| | ACCCACTTAAACTTCAAGTACCCAGCATTGAACGTGACTATGCCAAAC | |
| | AATGAACAATTTGACAAATTGTACATTTGGGGGGTTCACCACCCGGG | |
| | TACGGACAAGGACCAAATCTTCCTGTATGCTCAATCATCAGGAAGAA | |
| | TCACAGTATCTACCAAAAGAAGCCAACAAGCTGTAATCCCGAATATC | |
| | GGATCTAGACCCAGAATAAGGAATATCCCTAGCAGAATAAGCATCTA | |
| | TTGGACAATAGTAAAACCGGGAGACATACTTTTGATTAACAGCACAG | |
| | GGAATCTAATTGCTCCTAGGGGTTACTTCAAAATACGAAGTGGGAAA | |
| | AGCTCAATAATGAGATCAGATGCACCCATTGGCAAATGCAAGTCTGA | |
| | ATGCATCACTCCAAATGGAAGCATTCCCAATGACAAACCATTCCAAAA | |
| | TGTAAACAGGATCACATACGGGGCCTGTCCCAGATATGTTAAGCAAA | |
| | GCACTCTGAAATTGGCAACAGGAATGCGAAATGTACCAGAGAAACAA | |
| | ACTAGAGGCATATTTGGCGCAATAGCGGGTTTCATAGAAAATGGTTG | |
| | GGAGGGAATGGTGGATGGTTGGTACGGTTTCAGGCATCAAAATTCTG | |
| | AGGGAAGAGGACAAGCAGCAGATCTCAAAAGCACTCAAGCAGCAAT | |
| | CGATCAAATCAATGGGAAGCTGAATCGATTGATCGGGAAAACCAACG | |
| | AGAAATTCCATCAGATTGAGAAAGAATTCTCAGAAGTAGAAGGGAGA | |
| | ATTCAGGACCTTGAGAAATATGTTGAGGACACAAAAATAGATCTCTGG | |
| | TCATACAACGCAGAGCTTCTTGTTGCCCTGGAAAACCAACATACAATT | |
| | GATCTAACTGACTCAGAAATGAACAAACTGTTTGAAAAAACAAAGAAG | |
| | CAACTGAGGGAAAATGCTGAGGATATGGGCAATGGTTGTTTCAAAAT | |
| | ATACCACAAATGTGACAATGCCTGCATAGGATCAATCAGAAATGGAA | |
| | CTTATGACCACGATGTATACAGGGATGAAGCATTAAACAACCGGTTC | |
| | CAGATCAAGGGAGTTGAGCTGAAGTCAGGGTACAAAGATTGGATCCT | |
| | ATGGATTTCCTTTGCCATATCATGTTTTTTGCTTTGTGTTGCTTTGTTG | |
| | GGGTTCATCATGTGGGCCTGCCAAAAGGGCAACATTAGGTGCAACAT | |
| | TTGCATTTGAGTGCATTAATTAAAAACACCCTTGTTTCTACTGGAAGC | |
| | GGAGCCACGAACTTCTCTCTGTTAAAGCAAGCAGGAGATGTTGAAGA | |
| | AAACCCCGGGCCTATTTTCTAATATCCACAAAATGAAGGCAATAATTG | |
| | TACTACTCATGGTAGTAACATCCAATGCAGATCGAATCTGCACTGGG | |
| | ATAACATCGTCAAACTCACCACATGTCGTCAAAACTGCTACTCAAGG | |
| | GGAGGTCAACGTGACCGGTGTAATACCACTGACAACAACACCCACCA | |
| | AATCTCATTTTGCAAATCTCAAAGGAACAGAAACCAGGGGGAAACTAT | |
| | GCCCAAAATGCCTCAACTGCACAGATCTGGATGTAGCCTTGGGCAGA | |
| | CCAAAATGCACAGGGAAAATACCCTCTGCAAGGGTTTCAATACTCCA | |
| | TGAAGTCAGACCTGTTACATCTGGGTGCTTTCCTATAATGCACGATAG | |
| | AACAAAAATTAGACAGCTGCCTAACCTTCTCCGAGGATACGAACATG | |
| | TCAGGTTATCAACTCACAACGTTATCAATGCAAAAGATGCACCAGGAA | |
| | GACCCTACGAAATTGGAACCTCAGGGTCTTGCCCTAACATTACCAAT | |
| | GGAAACGGATTCTTCGCAACAATGGCTTGGGCCGTCCCAAAAAACAA | |
| | AACAGCAACAAATCCATTAACAATAGAAGTACCATACATTTGTACAGA | |
| | AGGAGAAGACCAAATTACCGTTTGGGGGTTCCACTCTGACAACGAGA | |
| | CCCAAATGGCAAAGCTCTATGGGGACTCAAAGCCCCAGAAGTTCACC | |
| | TCATCTGCCAACGGAGTGACCACACATTACGTTTCACAGATTGGTGG | |
| | CTTCCCAAATCAAACAGAAGACGGAGGACTACCACAAAGTGGCAGAA | |
| | TTGTTGTTGATTACATGGTGCAGAAATCTGGAAAAACAGGAACAATTA | |
| | CCTATCAAAGAGGTATTTTATTGCCTCAAAAAGTGTGGTGCGCAAGT | |
| | GGCAGGAGCAAGGTAATAAAAGGATCCTTGCCCTTAATTGGAGAAGC | |
| | AGATTGCCTCCATGAAAAATACGGTGGATTAAACAAAAGCAAGCCTTA | |
| | CTACACAGGGGAACATGCAAAGGCCATAGGAAATTGCCCAATATGGG | |
| | TGAAAACACCCTTGAAGCTGGCCAATGGAACCAAATATAGACCCCCT | |
| | GCAAAACTATTAAAGGAAAGAGGTTTCTTCGGAGCCATTGCTGGTTT | |
| | CTTAGAGGGAGGATGGGAAGGAATGATTGCAGGTTGGCACGGATAC | |
| | ACATCCCATGGGGCACATGGAGTAGCGGTGGCAGCTGACCTTAAGA | |
| | GCACTCAAGAGGCCATAAACAAGATAACAAAAAATCTCAACTCTTTGA | |
| | GTGAGCTGGAAGTAAAGAATCTTCAAAGACTAAGCGGTGCCATGGAT | |
| | GAACTCCACAACGAAATACTAGAACTAGATGAGAAAGTGGATGATCT | |
| | CAGAGCTGACACAATAAGCTCACAAATAGAACTCGCAGTCCTGCTTT | |
| | CCAATGAAGGAATAATAAACAGTGAAGATGAACATCTCTTGGCGCTT | |
| | GAAAGAAAGCTGAAGAAAATGCTGGGCCCCTCTGCTGTAGAGATAG | |
| | GGAATGGATGCTTTGAAACCAAACACAAGTGCAACCAGACCTGCCTC | |
| | GACAGAATAGCTGCTGGTACCTTTGATGCAGGAGAATTTTCTCTCCC | |
| | CACCTTTGATTCACTGAATATTACTGCTGCATCTTTAAATGACGACGG | |
| | ATTGGACAATCATACTATACTGCTTTACTACTCAACTGCTGCCTCCAG | |
| | TTTGGCTGTAACACTGATGATAGCTATCTTTGTTGTTTATATGGTCTC | |
| | CAGAGACAATGTTTCTTGCTCCATTTGTCTATAAGGAAAGTTAAGCCC | |
| | TGTATTTTCCTTTATTGTAGTGCTTGTTTGCTTGTTGTCATTACAAAGA | |
| | AACGTTATTGAAAAATTGATGA | |
| Pan-CoV-Flu | ATGTTCGTGTTCCTGGTGCTGCTGCCCCTGGTGAGCAGCCAGTGCG | 417 |
| Vaccine #29 | TGAACCTGACCACCAGGACCCAGCTGCCCCCCGCCTACACCAACAG | |
| CoV-Wuhan | CTTCACCAGGGGCGTGTACTACCCCGACAAGGTGTTCAGGAGCAGC | |
| Spike(HexaPro)- | GTGCTGCACAGCACCCAGGACCTGTTCCTGCCCTTCTTCAGCAACGT | |
| Influenza (HA) | GACCTGGTTCCACGCCATCCACGTGAGCGGCACCAACGGCACCAAG | |
| | AGGTTCGACAACCCCGTGCTGCCCTTCAACGACGGCGTGTACTTCG | |
| | CCAGCACCGAGAAGAGCAACATCATCAGGGGCTGGATCTTCGGCAC | |
| | CACCCTGGACAGCAAGACCCAGAGCCTGCTGATCGTGAACAACGCC | |
| | ACCAACGTGGTGATCAAGGTGTGCGAGTTCCAGTTCTGCAACGACCC | |
| | CTTCCTGGGCGTGTACTACCACAAGAACAACAAGAGCTGGATGGAGA | |
| | GCGAGTTCAGGGTGTACAGCAGCGCCAACAACTGCACCTTCGAGTA | |
| | CGTGAGCCAGCCCTTCCTGATGGACCTGGAGGGCAAGCAGGGCAAC | |
| | TTCAAGAACCTGAGGGAGTTCGTGTTCAAGAACATCGACGGCTACTT | |
| | CAAGATCTACAGCAAGCACACCCCCATCAACCTGGTGAGGGACCTG | |
| | CCCCAGGGCTTCAGCGCCCTGGAGCCCCTGGTGGACCTGCCCATCG | |
| | GCATCAACATCACCAGGTTCCAGACCCTGCTGGCCCTGCACAGGAG | |
| | CTACCTGACCCCCGGCGACAGCAGCAGCGGCTGGACCGCCGGCGC | |
| | CGCCGCCTACTACGTGGGCTACCTGCAGCCCAGGACCTTCCTGCTG | |
| | AAGTACAACGAGAACGGCACCATCACCGACGCCGTGGACTGCGCCC | |
| | TGGACCCCCTGAGCGAGACCAAGTGCACCCTGAAGAGCTTCACCGT | |
| | GGAGAAGGGCATCTACCAGACCAGCAACTTCAGGGTGCAGCCCACC | |
| | GAGAGCATCGTGAGGTTCCCCAACATCACCAACCTGTGCCCCTTCG | |
| | GCGAGGTGTTCAACGCCACCAGGTTCGCCAGCGTGTACGCCTGGAA | |
| | CAGGAAGAGGATCAGCAACTGCGTGGCCGACTACAGCGTGCTGTAC | |
| | AACAGCGCCAGCTTCAGCACCTTCAAGTGCTACGGCGTGAGCCCCA | |
| | CCAAGCTGAACGACCTGTGCTTCACCAACGTGTACGCCGACAGCTTC | |
| | GTGATCAGGGGCGACGAGGTGAGGCAGATCGCCCCCGGCCAGACC | |
| | GGCAAGATCGCCGACTACAACTACAAGCTGCCCGACGACTTCACCG | |
| | GCTGCGTGATCGCCTGGAACAGCAACAACCTGGACAGCAAGGTGGG | |
| | CGGCAACTACAACTACCTGTACAGGCTGTTCAGGAAGAGCAACCTGA | |
| | AGCCCTTCGAGAGGGACATCAGCACCGAGATCTACCAGGCCGGCAG | |
| | CACCCCCTGCAACGGCGTGGAGGGCTTCAACTGCTACTTCCCCCTG | |
| | CAGAGCTACGGCTTCCAGCCCACCAACGGCGTGGGCTACCAGCCCT | |
| | ACAGGGTGGTGGTGCTGAGCTTCGAGCTGCTGCACGCCCCCGCCAC | |
| | CGTGTGCGGCCCCAAGAAGAGCACCAACCTGGTGAAGAACAAGTGC | |
| | GTGAACTTCAACTTCAACGGCCTGACCGGCACCGGCGTGCTGACCG | |
| | AGAGCAACAAGAAGTTCCTGCCCTTCCAGCAGTTCGGCAGGGACAT | |
| | CGCCGACACCACCGACGCCGTGAGGGACCCCCAGACCCTGGAGAT | |
| | CCTGGACATCACCCCCTGCAGCTTCGGCGGCGTGAGCGTGATCACC | |
| | CCCGGCACCAACACCAGCAACCAGGTGGCCGTGCTGTACCAGGACG | |
| | TGAACTGCACCGAGGTGCCCGTGGCCATCCACGCCGACCAGCTGAC | |
| | CCCCACCTGGAGGGTGTACAGCACCGGCAGCAACGTGTTCCAGACC | |
| | AGGGCCGGCTGCCTGATCGGCGCCGAGCACGTGAACAACAGCTAC | |
| | GAGTGCGACATCCCCATCGGCGCCGGCATCTGCGCCAGCTACCAGA | |
| | CCCAGACCAACAGCCCCGGCAGCGCCAGCAGCGTGGCCAGCCAGA | |
| | GCATCATCGCCTACACCATGAGCCTGGGCGCCGAGAACAGCGTGGC | |
| | CTACAGCAACAACAGCATCGCCATCCCCACCAACTTCACCATCAGCG | |
| | TGACCACCGAGATCCTGCCCGTGAGCATGACCAAGACCAGCGTGGA | |
| | CTGCACCATGTACATCTGCGGCGACAGCACCGAGTGCAGCAACCTG | |
| | CTGCTGCAGTACGGCAGCTTCTGCACCCAGCTGAACAGGGCCCTGA | |
| | CCGGCATCGCCGTGGAGCAGGACAAGAACACCCAGGAGGTGTTCGC | |
| | CCAGGTGAAGCAGATCTACAAGACCCCCCCCATCAAGGACTTCGGC | |
| | GGCTTCAACTTCAGCCAGATCCTGCCCGACCCCAGCAAGCCCAGCA | |
| | AGAGGAGCCCCATCGAGGACCTGCTGTTCAACAAGGTGACCCTGGC | |
| | CGACGCCGGCTTCATCAAGCAGTACGGCGACTGCCTGGGCGACATC | |
| | GCCGCCAGGGACCTGATCTGCGCCCAGAAGTTCAACGGCCTGACCG | |
| | TGCTGCCCCCCCTGCTGACCGACGAGATGATCGCCCAGTACACCAG | |
| | CGCCCTGCTGGCCGGCACCATCACCAGCGGCTGGACCTTCGGCGC | |
| | CGGCCCCGCCCTGCAGATCCCCTTCCCCATGCAGATGGCCTACAGG | |
| | TTCAACGGCATCGGCGTGACCCAGAACGTGCTGTACGAGAACCAGA | |
| | AGCTGATCGCCAACCAGTTCAACAGCGCCATCGGCAAGATCCAGGA | |
| | CAGCCTGAGCAGCACCCCCAGCGCCCTGGGCAAGCTGCAGGACGT | |
| | GGTGAACCAGAACGCCCAGGCCCTGAACACCCTGGTGAAGCAGCTG | |
| | AGCAGCAACTTCGGCGCCATCAGCAGCGTGCTGAACGACATCCTGA | |
| | GCAGGCTGGACCCCCCCGAGGCCGAGGTGCAGATCGACAGGCTGA | |
| | TCACCGGCAGGCTGCAGAGCCTGCAGACCTACGTGACCCAGCAGCT | |
| | GATCAGGGCCGCCGAGATCAGGGCCAGCGCCAACCTGGCCGCCAC | |
| | CAAGATGAGCGAGTGCGTGCTGGGCCAGAGCAAGAGGGTGGACTTC | |
| | TGCGGCAAGGGCTACCACCTGATGAGCTTCCCCCAGAGCGCCCCCC | |
| | ACGGCGTGGTGTTCCTGCACGTGACCTACGTGCCCGCCCAGGAGAA | |
| | GAACTTCACCACCGCCCCCGCCATCTGCCACGACGGCAAGGCCCAC | |
| | TTCCCCAGGGAGGGCGTGTTCGTGAGCAACGGCACCCACTGGTTCG | |
| | TGACCCAGAGGAACTTCTACGAGCCCCAGATCATCACCACCGACAAC | |
| | ACCTTCGTGAGCGGCAACTGCGACGTGGTGATCGGCATCGTGAACA | |
| | ACACCGTGTACGACCCCCTGCAGCCCGAGCTGGACAGCTTCAAGGA | |
| | GGAGCTGGACAAGTACTTCAAGAACCACACCAGCCCCGACGTGGAC | |
| | CTGGGCGACATCAGCGGCATCAACGCCAGCGTGGTGAACATCCAGA | |
| | AGGAGATCGACAGGCTGAACGAGGTGGCCAAGAACCTGAACGAGAG | |
| | CCTGATCGACCTGCAGGAGCTGGGCAAGTACGAGCAGTACATCAAG | |
| | TGGCCCTGGTACATCTGGCTGGGCTTCATCGCCGGCCTGATCGCCA | |
| | TCGTGATGGTGACCATCATGCTGTGCTGCATGACCAGCTGCTGCAGC | |
| | TGCCTGAAGGGCTGCTGCAGCTGCGGCAGCTGCTGCAAGTTCGACG | |
| | AGGACGACAGCGAGCCCGTGCTGAAGGGCGTGAAGCTGCACTACAC | |
| | CGGAAGCGGAGCCACGAACTTCTCTCTGTTAAAGCAAGCAGGAGAT | |
| | GTTGAAGAAAACCCCGGGCCTATGAAGGCAATACTAGTAGTTCTGCT | |
| | ATATACATTTGCAACCGCAAATGCAGACACATTATGTATAGGTTATCA | |
| | TGCGAACAACTCAACAGACACTGTAGACACAGTACTAGAAAAGAATG | |
| | TAACAGTAACACACTCTGTTAACCTTCTAGAAGACAAGCATAACGGGA | |
| | AACTATGCAAACTAAGAGGGGTAGCCCCATTGCATTTGGGTAAATGT | |
| | AACATTGCTGGCTGGATCCTGGGAAATCCAGAGTGTGAATCACTCTC | |
| | CACAGCAAGCTCATGGTCCTACATTGTGGAAACATCTAGTTCAGACA | |
| | ATGGAACGTGTTACCCAGGAGATTTCATCGATTATGAGGAGCTAAGA | |
| | GAGCAATTGAGCTCAGTGTCATCATTTGAAAGGTTTGAGATATTCCCC | |
| | AAGACAAGTTCATGGCCCAATCATGACTCGAACAAAGGTGTAACGAC | |
| | AGCATGTCCTCATGCTGGAGCAAAAAGCTTCTACAAAAATTTAATATG | |
| | GCTAGTTAAAAAAGGAAATTCATACCCAAAGCTCAGCAAATCCTAGAT | |
| | TAATGATAAAGGGAAAGAAGTCCTCGTGCTATGGGGCATTCACCATC | |
| | CACCTACTAGTGCTGACCAACAAAGTCTCTATCAGAATGCAGATGCA | |
| | TATGTTTTTGTGGGGACATCAAGATACAGCAAGAAGTTCAAGCCGGA | |
| | AATAGCAATAAGGCCCAAAGTGAGGGATCAAGAAGGGAGAATGAACT | |
| | ATTACTGGACACTAGTAGAGCCGGGAGACAAAATAACATTCGAAGCA | |
| | ACTGGAAATCTAGTGGTACCGAGATATGCATTCGCAATGGAAAGAAA | |
| | TGCTGGATCTGGTATTATCATTTCAGATACACCAGTCCACGATTGCAA | |
| | TACAACTTGTCAGACACCCAAGGGTGCTATAAACACCAGCCTCCCAT | |
| | TTCAGAACATACATCCGATCACAATTGGAAAATGTCCAAAATATGTAA | |
| | AAAGCACAAAATTGAGACTGGCCACAGGATTGAGGAATGTCCCGTCC | |
| | ATTCAATCTAGAGGCCTATTTGGGGCCATTGCCGGTTTCATTGAAGG | |
| | GGGGTGGACAGGGATGGTAGATGGATGGTACGGTTATCACCATCAA | |
| | AATGAGCAGGGGTCAGGATATGCAGCCGACCTGAAGAGCACACAGA | |
| | ATGCCATTGACGAGATTACTAACAAAGTAAACTCTGTTATTGAAAAAA | |
| | TGAATACACAGTTCACAGCAGTAGGTAAAGAGTTCAACCACCTGGAA | |
| | AAAAGAATAGAGAATTTAAATAAAAAAGTTGATGATGGTTTCCTGGAC | |
| | ATTTGGACTTACAATGCCGAACTGTTGGTTCTATTGGAAAATGAAAGA | |
| | ACTTTGGACTACCACGATTCAAAGGTGAAGAACTTATATGAAAAGGTA | |
| | AGAAGCCAGTTAAAAAACAATGCCAAGGAAATTGGAAACGGCTGCTT | |
| | TGAATTTTACCACAAATGTGATAACACGTGCATGGAAAGTGTCAAAAA | |
| | TGGGACTTATGACTACCCAAAATACTCAGAGGAAGCAAAATTAAACA | |
| | GAGAAGAAATAGATGGGGTAAAGCTGGAATCAACAAGGATTTACCAG | |
| | ATTTTGGCGATCTATTCAACCGTCGCCAGTTCATTGGTACTGGTAGTC | |
| | TCCCTGGGGGCAATCAGTTTCTGGATGTGCTCTAATGGGTCTCTACA | |
| | GTGTAGAATATGTATTTAAGGAAGCGGAGCCACGAACTTCTCTCTGTT | |
| | AAAGCAAGCAGGAGATGTTGAAGAAAACCCCGGGCCTAGCAAAAGC | |
| | AGGGGATAATTCTATTAACCATGAAGACTATCATTGCTTTGAGCTACA | |
| | TTCTATGTCTGGTTTTCGCTCAAAAACTTCCTGGAAATGACAATAGCA | |
| | CTGCAACGCTGTGCCTTGGGCACCATGCAGTACCAAACGGAACGAT | |
| | AGTGAAAACAATCACGAATGACCAAATTGAAGTTACTAATGCTACTGA | |
| | GCTGGTTCAGAATTCCTCAATAGGTGAAATATGCGACAGTCCTCATC | |
| | AGATCCTTGATGGAGAAAACTGCACACTAATAGATGCTCTATTGGGA | |
| | GACCCTCAGTGTGATGGCTTTCAAAATAAGAAATGGGACCTTTTTGTT | |
| | GAACGAAGCAAAGCCTACAGCAACTGTTACCCTTATGATGTGCCGGA | |
| | TTATGCCTCCCTTAGGTCACTAGTTGCCTCATCCGGCACACTGGAGT | |
| | TTAACAATGAAAGCTTCAATTGGGCTGGAGTCACTCAAAACGGAACA | |
| | AGTTCTGCTTGCATAAGGGGATCTAATAGTAGTTTCTTTAGTAGATTA | |
| | AATTGGTTGACCCACTTAAACTTCAAGTACCCAGCATTGAACGTGACT | |
| | ATGCCAAACAATGAACAATTTGACAAATTGTACATTTGGGGGGTTCAC | |
| | CACCCGGGTACGGACAAGGACCAAATCTTCCTGTATGCTCAATCATC | |
| | AGGAAGAATCACAGTATCTACCAAAAGAAGCCAACAAGCTGTAATCC | |
| | CGAATATCGGATCTAGACCCAGAATAAGGAATATCCCTAGCAGAATA | |
| | AGCATCTATTGGACAATAGTAAAACCGGGAGACATACTTTTGATTAAC | |
| | AGCACAGGGAATCTAATTGCTCCTAGGGGTTACTTCAAAATACGAAG | |
| | TGGGAAAAGCTCAATAATGAGATCAGATGCACCCATTGGCAAATGCA | |
| | AGTCTGAATGCATCACTCCAAATGGAAGCATTCCCAATGACAAACCAT | |
| | TCCAAAATGTAAACAGGATCACATACGGGGCCTGTCCCAGATATGTT | |
| | AAGCAAAGCACTCTGAAATTGGCAACAGGAATGCGAAATGTACCAGA | |
| | GAAACAAACTAGAGGCATATTTGGCGCAATAGCGGGTTTCATAGAAA | |
| | ATGGTTGGGAGGGAATGGTGGATGGTTGGTACGGTTTCAGGCATCA | |
| | AAATTCTGAGGGAAGAGGACAAGCAGCAGATCTCAAAAGCACTCAAG | |
| | CAGCAATCGATCAAATCAATGGGAAGCTGAATCGATTGATCGGGAAA | |
| | ACCAACGAGAAATTCCATCAGATTGAGAAAGAATTCTCAGAAGTAGAA | |
| | GGGAGAATTCAGGACCTTGAGAAATATGTTGAGGACACAAAAATAGA | |
| | TCTCTGGTCATACAACGCAGAGCTTCTTGTTGCCCTGGAAAACCAAC | |
| | ATACAATTGATCTAACTGACTCAGAAATGAACAAACTGTTTGAAAAAA | |
| | CAAAGAAGCAACTGAGGGAAAATGCTGAGGATATGGGCAATGGTTGT | |
| | TTCAAAATATACCACAAATGTGACAATGCCTGCATAGGATCAATCAGA | |
| | AATGGAACTTATGACCACGATGTATACAGGGATGAAGCATTAAACAA | |
| | CCGGTTCCAGATCAAGGGAGTTGAGCTGAAGTCAGGGTACAAAGATT | |
| | GGATCCTATGGATTTCCTTTGCCATATCATGTTTTTTGCTTTGTGTTGC | |
| | TTTGTTGGGGTTCATCATGTGGGCCTGCCAAAAGGGCAACATTAGGT | |
| | GCAACATTTGCATTTGAGTGCATTAATTAAAAACACCCTTGTTTCTACT | |
| | GGAAGCGGAGCCACGAACTTCTCTCTGTTAAAGCAAGCAGGAGATGT | |
| | TGAAGAAAACCCCGGGCCTATTTTCTAATATCCACAAAATGAAGGCAA | |
| | TAATTGTACTACTCATGGTAGTAACATCCAATGCAGATCGAATCTGCA | |
| | CTGGGATAACATCGTCAAACTCACCACATGTCGTCAAAACTGCTACT | |
| | CAAGGGGAGGTCAACGTGACCGGTGTAATACCACTGACAACAACAC | |
| | CCACCAAATCTCATTTTGCAAATCTCAAAGGAACAGAAACCAGGGGG | |
| | AAACTATGCCCAAAATGCCTCAACTGCACAGATCTGGATGTAGCCTT | |
| | GGGCAGACCAAAATGCACAGGGAAAATACCCTCTGCAAGGGTTTCAA | |
| | TACTCCATGAAGTCAGACCTGTTACATCTGGGTGCTTTCCTATAATGC | |
| | ACGATAGAACAAAAATTAGACAGCTGCCTAACCTTCTCCGAGGATAC | |
| | GAACATGTCAGGTTATCAACTCACAACGTTATCAATGCAAAAGATGCA | |
| | CCAGGAAGACCCTACGAAATTGGAACCTCAGGGTCTTGCCCTAACAT | |
| | TACCAATGGAAACGGATTCTTCGCAACAATGGCTTGGGCCGTCCCAA | |
| | AAAACAAAACAGCAACAAATCCATTAACAATAGAAGTACCATACATTT | |
| | GTACAGAAGGAGAAGACCAAATTACCGTTTGGGGGTTCCACTCTGAC | |
| | AACGAGACCCAAATGGCAAAGCTCTATGGGGACTCAAAGCCCCAGA | |
| | AGTTCACCTCATCTGCCAACGGAGTGACCACACATTACGTTTCACAG | |
| | ATTGGTGGCTTCCCAAATCAAACAGAAGACGGAGGACTACCACAAAG | |
| | TGGCAGAATTGTTGTTGATTACATGGTGCAGAAATCTGGAAAAACAG | |
| | GAACAATTACCTATCAAAGAGGTATTTTATTGCCTCAAAAAGTGTGGT | |
| | GCGCAAGTGGCAGGAGCAAGGTAATAAAAGGATCCTTGCCCTTAATT | |
| | GGAGAAGCAGATTGCCTCCATGAAAAATACGGTGGATTAAACAAAAG | |
| | CAAGCCTTACTACACAGGGGAACATGCAAAGGCCATAGGAAATTGCC | |
| | CAATATGGGTGAAAACACCCTTGAAGCTGGCCAATGGAACCAAATAT | |
| | AGACCCCCTGCAAAACTATTAAAGGAAAGAGGTTTCTTCGGAGCCAT | |
| | TGCTGGTTTCTTAGAGGGAGGATGGGAAGGAATGATTGCAGGTTGG | |
| | CACGGATACACATCCCATGGGGCACATGGAGTAGCGGTGGCAGCTG | |
| | ACCTTAAGAGCACTCAAGAGGCCATAAACAAGATAACAAAAAATCTCA | |
| | ACTCTTTGAGTGAGCTGGAAGTAAAGAATCTTCAAAGACTAAGCGGT | |
| | GCCATGGATGAACTCCACAACGAAATACTAGAACTAGATGAGAAAGT | |
| | GGATGATCTCAGAGCTGACACAATAAGCTCACAAATAGAACTCGCAG | |
| | TCCTGCTTTCCAATGAAGGAATAATAAACAGTGAAGATGAACATCTCT | |
| | TGGCGCTTGAAAGAAAGCTGAAGAAAATGCTGGGCCCCTCTGCTGTA | |
| | GAGATAGGGAATGGATGCTTTGAAACCAAACACAAGTGCAACCAGAC | |
| | CTGCCTCGACAGAATAGCTGCTGGTACCTTTGATGCAGGAGAATTTT | |
| | CTCTCCCCACCTTTGATTCACTGAATATTACTGCTGCATCTTTAAATG | |
| | ACGACGGATTGGACAATCATACTATACTGCTTTACTACTCAACTGCTG | |
| | CCTCCAGTTTGGCTGTAACACTGATGATAGCTATCTTTGTTGTTTATA | |
| | TGGTCTCCAGAGACAATGTTTCTTGCTCCATTTGTCTATAAGGAAAGT | |
| | TAAGCCCTGTATTTTCCTTTATTGTAGTGCTTGTTTGCTTGTTGTCATT | |
| | ACAAAGAAACGTTATTGAAAAATTGATGA | |
| Pan-CoV-Flu | ATGTTCGTGTTCCTGGTGCTGCTGCCCCTGGTGAGCAGCCAGTGCG | 418 |
| Vaccine #30 | TGAACCTGACCACCAGGACCCAGCTGCCCCCCGCCTACACCAACAG | |
| CoV-Wuhan | CTTCACCAGGGGCGTGTACTACCCCGACAAGGTGTTCAGGAGCAGC | |
| Spike(2 proline | GTGCTGCACAGCACCCAGGACCTGTTCCTGCCCTTCTTCAGCAACGT | |
| substitution)- | GACCTGGTTCCACGCCATCCACGTGAGCGGCACCAACGGCACCAAG | |
| Influenza (HA) | AGGTTCGACAACCCCGTGCTGCCCTTCAACGACGGCGTGTACTTCG | |
| | CCAGCACCGAGAAGAGCAACATCATCAGGGGCTGGATCTTCGGCAC | |
| | CACCCTGGACAGCAAGACCCAGAGCCTGCTGATCGTGAACAACGCC | |
| | ACCAACGTGGTGATCAAGGTGTGCGAGTTCCAGTTCTGCAACGACCC | |
| | CTTCCTGGGCGTGTACTACCACAAGAACAACAAGAGCTGGATGGAGA | |
| | GCGAGTTCAGGGTGTACAGCAGCGCCAACAACTGCACCTTCGAGTA | |
| | CGTGAGCCAGCCCTTCCTGATGGACCTGGAGGGCAAGCAGGGCAAC | |
| | TTCAAGAACCTGAGGGAGTTCGTGTTCAAGAACATCGACGGCTACTT | |
| | CAAGATCTACAGCAAGCACACCCCCATCAACCTGGTGAGGGACCTG | |
| | CCCCAGGGCTTCAGCGCCCTGGAGCCCCTGGTGGACCTGCCCATCG | |
| | GCATCAACATCACCAGGTTCCAGACCCTGCTGGCCCTGCACAGGAG | |
| | CTACCTGACCCCCGGCGACAGCAGCAGCGGCTGGACCGCCGGCGC | |
| | CGCCGCCTACTACGTGGGCTACCTGCAGCCCAGGACCTTCCTGCTG | |
| | AAGTACAACGAGAACGGCACCATCACCGACGCCGTGGACTGCGCCC | |
| | TGGACCCCCTGAGCGAGACCAAGTGCACCCTGAAGAGCTTCACCGT | |
| | GGAGAAGGGCATCTACCAGACCAGCAACTTCAGGGTGCAGCCCACC | |
| | GAGAGCATCGTGAGGTTCCCCAACATCACCAACCTGTGCCCCTTCG | |
| | GCGAGGTGTTCAACGCCACCAGGTTCGCCAGCGTGTACGCCTGGAA | |
| | CAGGAAGAGGATCAGCAACTGCGTGGCCGACTACAGCGTGCTGTAC | |
| | AACAGCGCCAGCTTCAGCACCTTCAAGTGCTACGGCGTGAGCCCCA | |
| | CCAAGCTGAACGACCTGTGCTTCACCAACGTGTACGCCGACAGCTTC | |
| | GTGATCAGGGGCGACGAGGTGAGGCAGATCGCCCCCGGCCAGACC | |
| | GGCAAGATCGCCGACTACAACTACAAGCTGCCCGACGACTTCACCG | |
| | GCTGCGTGATCGCCTGGAACAGCAACAACCTGGACAGCAAGGTGGG | |
| | CGGCAACTACAACTACCTGTACAGGCTGTTCAGGAAGAGCAACCTGA | |
| | AGCCCTTCGAGAGGGACATCAGCACCGAGATCTACCAGGCCGGCAG | |
| | CACCCCCTGCAACGGCGTGGAGGGCTTCAACTGCTACTTCCCCCTG | |
| | CAGAGCTACGGCTTCCAGCCCACCAACGGCGTGGGCTACCAGCCCT | |
| | ACAGGGTGGTGGTGCTGAGCTTCGAGCTGCTGCACGCCCCCGCCAC | |
| | CGTGTGCGGCCCCAAGAAGAGCACCAACCTGGTGAAGAACAAGTGC | |
| | GTGAACTTCAACTTCAACGGCCTGACCGGCACCGGCGTGCTGACCG | |
| | AGAGCAACAAGAAGTTCCTGCCCTTCCAGCAGTTCGGCAGGGACAT | |
| | CGCCGACACCACCGACGCCGTGAGGGACCCCCAGACCCTGGAGAT | |
| | CCTGGACATCACCCCCTGCAGCTTCGGCGGCGTGAGCGTGATCACC | |
| | CCCGGCACCAACACCAGCAACCAGGTGGCCGTGCTGTACCAGGACG | |
| | TGAACTGCACCGAGGTGCCCGTGGCCATCCACGCCGACCAGCTGAC | |
| | CCCCACCTGGAGGGTGTACAGCACCGGCAGCAACGTGTTCCAGACC | |
| | AGGGCCGGCTGCCTGATCGGCGCCGAGCACGTGAACAACAGCTAC | |
| | GAGTGCGACATCCCCATCGGCGCCGGCATCTGCGCCAGCTACCAGA | |
| | CCCAGACCAACAGCCCCAGGAGGGCCAGGAGCGTGGCCAGCCAGA | |
| | GCATCATCGCCTACACCATGAGCCTGGGCGCCGAGAACAGCGTGGC | |
| | CTACAGCAACAACAGCATCGCCATCCCCACCAACTTCACCATCAGCG | |
| | TGACCACCGAGATCCTGCCCGTGAGCATGACCAAGACCAGCGTGGA | |
| | CTGCACCATGTACATCTGCGGCGACAGCACCGAGTGCAGCAACCTG | |
| | CTGCTGCAGTACGGCAGCTTCTGCACCCAGCTGAACAGGGCCCTGA | |
| | CCGGCATCGCCGTGGAGCAGGACAAGAACACCCAGGAGGTGTTCGC | |
| | CCAGGTGAAGCAGATCTACAAGACCCCCCCCATCAAGGACTTCGGC | |
| | GGCTTCAACTTCAGCCAGATCCTGCCCGACCCCAGCAAGCCCAGCA | |
| | AGAGGAGCTTCATCGAGGACCTGCTGTTCAACAAGGTGACCCTGGC | |
| | CGACGCCGGCTTCATCAAGCAGTACGGCGACTGCCTGGGCGACATC | |
| | GCCGCCAGGGACCTGATCTGCGCCCAGAAGTTCAACGGCCTGACCG | |
| | TGCTGCCCCCCCTGCTGACCGACGAGATGATCGCCCAGTACACCAG | |
| | CGCCCTGCTGGCCGGCACCATCACCAGCGGCTGGACCTTCGGCGC | |
| | CGGCGCCGCCCTGCAGATCCCCTTCGCCATGCAGATGGCCTACAGG | |
| | TTCAACGGCATCGGCGTGACCCAGAACGTGCTGTACGAGAACCAGA | |
| | AGCTGATCGCCAACCAGTTCAACAGCGCCATCGGCAAGATCCAGGA | |
| | CAGCCTGAGCAGCACCGCCAGCGCCCTGGGCAAGCTGCAGGACGT | |
| | GGTGAACCAGAACGCCCAGGCCCTGAACACCCTGGTGAAGCAGCTG | |
| | AGCAGCAACTTCGGCGCCATCAGCAGCGTGCTGAACGACATCCTGA | |
| | GCAGGCTGGACCCCCCCGAGGCCGAGGTGCAGATCGACAGGCTGA | |
| | TCACCGGCAGGCTGCAGAGCCTGCAGACCTACGTGACCCAGCAGCT | |
| | GATCAGGGCCGCCGAGATCAGGGCCAGCGCCAACCTGGCCGCCAC | |
| | CAAGATGAGCGAGTGCGTGCTGGGCCAGAGCAAGAGGGTGGACTTC | |
| | TGCGGCAAGGGCTACCACCTGATGAGCTTCCCCCAGAGCGCCCCCC | |
| | ACGGCGTGGTGTTCCTGCACGTGACCTACGTGCCCGCCCAGGAGAA | |
| | GAACTTCACCACCGCCCCCGCCATCTGCCACGACGGCAAGGCCCAC | |
| | TTCCCCAGGGAGGGCGTGTTCGTGAGCAACGGCACCCACTGGTTCG | |
| | TGACCCAGAGGAACTTCTACGAGCCCCAGATCATCACCACCGACAAC | |
| | ACCTTCGTGAGCGGCAACTGCGACGTGGTGATCGGCATCGTGAACA | |
| | ACACCGTGTACGACCCCCTGCAGCCCGAGCTGGACAGCTTCAAGGA | |
| | GGAGCTGGACAAGTACTTCAAGAACCACACCAGCCCCGACGTGGAC | |
| | CTGGGCGACATCAGCGGCATCAACGCCAGCGTGGTGAACATCCAGA | |
| | AGGAGATCGACAGGCTGAACGAGGTGGCCAAGAACCTGAACGAGAG | |
| | CCTGATCGACCTGCAGGAGCTGGGCAAGTACGAGCAGTACATCAAG | |
| | TGGCCCTGGTACATCTGGCTGGGCTTCATCGCCGGCCTGATCGCCA | |
| | TCGTGATGGTGACCATCATGCTGTGCTGCATGACCAGCTGCTGCAGC | |
| | TGCCTGAAGGGCTGCTGCAGCTGCGGCAGCTGCTGCAAGTTCGACG | |
| | AGGACGACAGCGAGCCCGTGCTGAAGGGCGTGAAGCTGCACTACAC | |
| | CGGAAGCGGAGCCACGAACTTCTCTCTGTTAAAGCAAGCAGGAGAT | |
| | GTTGAAGAAAACCCCGGGCCTATGAAGGCAATACTAGTAGTTCTGCT | |
| | ATATACATTTGCAACCGCAAATGCAGACACATTATGTATAGGTTATCA | |
| | TGCGAACAACTCAACAGACACTGTAGACACAGTACTAGAAAAGAATG | |
| | TAACAGTAACACACTCTGTTAACCTTCTAGAAGACAAGCATAACGGGA | |
| | AACTATGCAAACTAAGAGGGGTAGCCCCATTGCATTTGGGTAAATGT | |
| | AACATTGCTGGCTGGATCCTGGGAAATCCAGAGTGTGAATCACTCTC | |
| | CACAGCAAGCTCATGGTCCTACATTGTGGAAACATCTAGTTCAGACA | |
| | ATGGAACGTGTTACCCAGGAGATTTCATCGATTATGAGGAGCTAAGA | |
| | GAGCAATTGAGCTCAGTGTCATCATTTGAAAGGTTTGAGATATTCCCC | |
| | AAGACAAGTTCATGGCCCAATCATGACTCGAACAAAGGTGTAACGAC | |
| | AGCATGTCCTCATGCTGGAGCAAAAAGCTTCTACAAAAATTTAATATG | |
| | GCTAGTTAAAAAAGGAAATTCATACCCAAAGCTCAGCAAATCCTACAT | |
| | TAATGATAAAGGGAAAGAAGTCCTCGTGCTATGGGGCATTCACCATC | |
| | CACCTACTAGTGCTGACCAACAAAGTCTCTATCAGAATGCAGATGCA | |
| | TATGTTTTTGTGGGGACATCAAGATACAGCAAGAAGTTCAAGCCGGA | |
| | AATAGCAATAAGGCCCAAAGTGAGGGATCAAGAAGGGAGAATGAACT | |
| | ATTACTGGACACTAGTAGAGCCGGGAGACAAAATAACATTCGAAGCA | |
| | ACTGGAAATCTAGTGGTACCGAGATATGCATTCGCAATGGAAAGAAA | |
| | TGCTGGATCTGGTATTATCATTTCAGATACACCAGTCCACGATTGCAA | |
| | TACAACTTGTCAGACACCCAAGGGTGCTATAAACACCAGCCTCCCAT | |
| | TTCAGAACATACATCCGATCACAATTGGAAAATGTCCAAAATATGTAA | |
| | AAAGCACAAAATTGAGACTGGCCACAGGATTGAGGAATGTCCCGTCC | |
| | ATTCAATCTAGAGGCCTATTTGGGGCCATTGCCGGTTTCATTGAAGG | |
| | GGGGTGGACAGGGATGGTAGATGGATGGTACGGTTATCACCATCAA | |
| | AATGAGCAGGGGTCAGGATATGCAGCCGACCTGAAGAGCACACAGA | |
| | ATGCCATTGACGAGATTACTAACAAAGTAAACTCTGTTATTGAAAAAA | |
| | TGAATACACAGTTCACAGCAGTAGGTAAAGAGTTCAACCACCTGGAA | |
| | AAAAGAATAGAGAATTTAAATAAAAAAGTTGATGATGGTTTCCTGGAC | |
| | ATTTGGACTTACAATGCCGAACTGTTGGTTCTATTGGAAAATGAAAGA | |
| | ACTTTGGACTACCACGATTCAAAGGTGAAGAACTTATATGAAAAGGTA | |
| | AGAAGCCAGTTAAAAAACAATGCCAAGGAAATTGGAAACGGCTGCTT | |
| | TGAATTTTACCACAAATGTGATAACACGTGCATGGAAAGTGTCAAAAA | |
| | TGGGACTTATGACTACCCAAAATACTCAGAGGAAGCAAAATTAAACA | |
| | GAGAAGAAATAGATGGGGTAAAGCTGGAATCAACAAGGATTTACCAG | |
| | ATTTTGGCGATCTATTCAACCGTCGCCAGTTCATTGGTACTGGTAGTC | |
| | TCCCTGGGGGCAATCAGTTTCTGGATGTGCTCTAATGGGTCTCTACA | |
| | GTGTAGAATATGTATTTAAGGAAGCGGAGCCACGAACTTCTCTCTGTT | |
| | AAAGCAAGCAGGAGATGTTGAAGAAAACCCCGGGCCTAGCAAAAGC | |
| | AGGGGATAATTCTATTAACCATGAAGACTATCATTGCTTTGAGCTACA | |
| | TTCTATGTCTGGTTTTCGCTCAAAAACTTCCTGGAAATGACAATAGCA | |
| | CTGCAACGCTGTGCCTTGGGCACCATGCAGTACCAAACGGAACGAT | |
| | AGTGAAAACAATCACGAATGACCAAATTGAAGTTACTAATGCTACTGA | |
| | GCTGGTTCAGAATTCCTCAATAGGTGAAATATGCGACAGTCCTCATC | |
| | AGATCCTTGATGGAGAAAACTGCACACTAATAGATGCTCTATTGGGA | |
| | GACCCTCAGTGTGATGGCTTTCAAAATAAGAAATGGGACCTTTTTGTT | |
| | GAACGAAGCAAAGCCTACAGCAACTGTTACCCTTATGATGTGCCGGA | |
| | TTATGCCTCCCTTAGGTCACTAGTTGCCTCATCCGGCACACTGGAGT | |
| | TTAACAATGAAAGCTTCAATTGGGCTGGAGTCACTCAAAACGGAACA | |
| | AGTTCTGCTTGCATAAGGGGATCTAATAGTAGTTTCTTTAGTAGATTA | |
| | AATTGGTTGACCCACTTAAACTTCAAGTACCCAGCATTGAACGTGACT | |
| | ATGCCAAACAATGAACAATTTGACAAATTGTACATTTGGGGGGTTCAC | |
| | CACCCGGGTACGGACAAGGACCAAATCTTCCTGTATGCTCAATCATC | |
| | AGGAAGAATCACAGTATCTACCAAAAGAAGCCAACAAGCTGTAATCC | |
| | CGAATATCGGATCTAGACCCAGAATAAGGAATATCCCTAGCAGAATA | |
| | AGCATCTATTGGACAATAGTAAAACCGGGAGACATACTTTTGATTAAC | |
| | AGCACAGGGAATCTAATTGCTCCTAGGGGTTACTTCAAAATACGAAG | |
| | TGGGAAAAGCTCAATAATGAGATCAGATGCACCCATTGGCAAATGCA | |
| | AGTCTGAATGCATCACTCCAAATGGAAGCATTCCCAATGACAAACCAT | |
| | TCCAAAATGTAAACAGGATCACATACGGGGCCTGTCCCAGATATGTT | |
| | AAGCAAAGCACTCTGAAATTGGCAACAGGAATGCGAAATGTACCAGA | |
| | GAAACAAACTAGAGGCATATTTGGCGCAATAGCGGGTTTCATAGAAA | |
| | ATGGTTGGGAGGGAATGGTGGATGGTTGGTACGGTTTCAGGCATCA | |
| | AAATTCTGAGGGAAGAGGACAAGCAGCAGATCTCAAAAGCACTCAAG | |
| | CAGCAATCGATCAAATCAATGGGAAGCTGAATCGATTGATCGGGAAA | |
| | ACCAACGAGAAATTCCATCAGATTGAGAAAGAATTCTCAGAAGTAGAA | |
| | GGGAGAATTCAGGACCTTGAGAAATATGTTGAGGACACAAAAATAGA | |
| | TCTCTGGTCATACAACGCAGAGCTTCTTGTTGCCCTGGAAAACCAAC | |
| | ATACAATTGATCTAACTGACTCAGAAATGAACAAACTGTTTGAAAAAA | |
| | CAAAGAAGCAACTGAGGGAAAATGCTGAGGATATGGGCAATGGTTGT | |
| | TTCAAAATATACCACAAATGTGACAATGCCTGCATAGGATCAATCAGA | |
| | AATGGAACTTATGACCACGATGTATACAGGGATGAAGCATTAAACAA | |
| | CCGGTTCCAGATCAAGGGAGTTGAGCTGAAGTCAGGGTACAAAGATT | |
| | GGATCCTATGGATTTCCTTTGCCATATCATGTTTTTTGCTTTGTGTTGC | |
| | TTTGTTGGGGTTCATCATGTGGGCCTGCCAAAAGGGCAACATTAGGT | |
| | GCAACATTTGCATTTGAGTGCATTAATTAAAAACACCCTTGTTTCTACT | |
| | GGAAGCGGAGCCACGAACTTCTCTCTGTTAAAGCAAGCAGGAGATGT | |
| | TGAAGAAAACCCCGGGCCTATTTTCTAATATCCACAAAATGAAGGCAA | |
| | TAATTGTACTACTCATGGTAGTAACATCCAATGCAGATCGAATCTGCA | |
| | CTGGGATAACATCGTCAAACTCACCACATGTCGTCAAAACTGCTACT | |
| | CAAGGGGAGGTCAACGTGACCGGTGTAATACCACTGACAACAACAC | |
| | CCACCAAATCTCATTTTGCAAATCTCAAAGGAACAGAAACCAGGGGG | |
| | AAACTATGCCCAAAATGCCTCAACTGCACAGATCTGGATGTAGCCTT | |
| | GGGCAGACCAAAATGCACAGGGAAAATACCCTCTGCAAGGGTTTCAA | |
| | TACTCCATGAAGTCAGACCTGTTACATCTGGGTGCTTTCCTATAATGC | |
| | ACGATAGAACAAAAATTAGACAGCTGCCTAACCTTCTCCGAGGATAC | |
| | GAACATGTCAGGTTATCAACTCACAACGTTATCAATGCAAAAGATGCA | |
| | CCAGGAAGACCCTACGAAATTGGAACCTCAGGGTCTTGCCCTAACAT | |
| | TACCAATGGAAACGGATTCTTCGCAACAATGGCTTGGGCCGTCCCAA | |
| | AAAACAAAACAGCAACAAATCCATTAACAATAGAAGTACCATACATTT | |
| | GTACAGAAGGAGAAGACCAAATTACCGTTTGGGGGTTCCACTCTGAC | |
| | AACGAGACCCAAATGGCAAAGCTCTATGGGGACTCAAAGCCCCAGA | |
| | AGTTCACCTCATCTGCCAACGGAGTGACCACACATTACGTTTCACAG | |
| | ATTGGTGGCTTCCCAAATCAAACAGAAGACGGAGGACTACCACAAAG | |
| | TGGCAGAATTGTTGTTGATTACATGGTGCAGAAATCTGGAAAAACAG | |
| | GAACAATTACCTATCAAAGAGGTATTTTATTGCCTCAAAAAGTGTGGT | |
| | GCGCAAGTGGCAGGAGCAAGGTAATAAAAGGATCCTTGCCCTTAATT | |
| | GGAGAAGCAGATTGCCTCCATGAAAAATACGGTGGATTAAACAAAAG | |
| | CAAGCCTTACTACACAGGGGAACATGCAAAGGCCATAGGAAATTGCC | |
| | CAATATGGGTGAAAACACCCTTGAAGCTGGCCAATGGAACCAAATAT | |
| | AGACCCCCTGCAAAACTATTAAAGGAAAGAGGTTTCTTCGGAGCCAT | |
| | TGCTGGTTTCTTAGAGGGAGGATGGGAAGGAATGATTGCAGGTTGG | |
| | CACGGATACACATCCCATGGGGCACATGGAGTAGCGGTGGCAGCTG | |
| | ACCTTAAGAGCACTCAAGAGGCCATAAACAAGATAACAAAAAATCTCA | |
| | ACTCTTTGAGTGAGCTGGAAGTAAAGAATCTTCAAAGACTAAGCGGT | |
| | GCCATGGATGAACTCCACAACGAAATACTAGAACTAGATGAGAAAGT | |
| | GGATGATCTCAGAGCTGACACAATAAGCTCACAAATAGAACTCGCAG | |
| | TCCTGCTTTCCAATGAAGGAATAATAAACAGTGAAGATGAACATCTCT | |
| | TGGCGCTTGAAAGAAAGCTGAAGAAAATGCTGGGCCCCTCTGCTGTA | |
| | GAGATAGGGAATGGATGCTTTGAAACCAAACACAAGTGCAACCAGAC | |
| | CTGCCTCGACAGAATAGCTGCTGGTACCTTTGATGCAGGAGAATTTT | |
| | CTCTCCCCACCTTTGATTCACTGAATATTACTGCTGCATCTTTAAATG | |
| | ACGACGGATTGGACAATCATACTATACTGCTTTACTACTCAACTGCTG | |
| | CCTCCAGTTTGGCTGTAACACTGATGATAGCTATCTTTGTTGTTTATA | |
| | TGGTCTCCAGAGACAATGTTTCTTGCTCCATTTGTCTATAAGGAAAGT | |
| | TAAGCCCTGTATTTTCCTTTATTGTAGTGCTTGTTTGCTTGTTGTCATT | |
| | ACAAAGAAACGTTATTGAAAAATTGATGA | |
| Pan-CoV-Flu | ATGATGAAGGCAATACTAGTAGTTCTGCTATATACATTTGCAACCGCA | 419 |
| Vaccine #31 | AATGCAGACACATTATGTATAGGTTATCATGCGAACAACTCAACAGAC | |
| (H1N1) | ACTGTAGACACAGTACTAGAAAAGAATGTAACAGTAACACACTCTGTT | |
| | AACCTTCTAGAAGACAAGCATAACGGGAAACTATGCAAACTAAGAGG | |
| | GGTAGCCCCATTGCATTTGGGTAAATGTAACATTGCTGGCTGGATCC | |
| | TGGGAAATCCAGAGTGTGAATCACTCTCCACAGCAAGCTCATGGTCC | |
| | TACATTGTGGAAACATCTAGTTCAGACAATGGAACGTGTTACCCAGG | |
| | AGATTTCATCGATTATGAGGAGCTAAGAGAGCAATTGAGCTCAGTGT | |
| | CATCATTTGAAAGGTTTGAGATATTCCCCAAGACAAGTTCATGGCCCA | |
| | ATCATGACTCGAACAAAGGTGTAACGACAGCATGTCCTCATGCTGGA | |
| | GCAAAAAGCTTCTACAAAAATTTAATATGGCTAGTTAAAAAAGGAAAT | |
| | TCATACCCAAAGCTCAGCAAATCCTACATTAATGATAAAGGGAAAGAA | |
| | GTCCTCGTGCTATGGGGCATTCACCATCCACCTACTAGTGCTGACCA | |
| | ACAAAGTCTCTATCAGAATGCAGATGCATATGTTTTTGTGGGGACATC | |
| | AAGATACAGCAAGAAGTTCAAGCCGGAAATAGCAATAAGGCCCAAAG | |
| | TGAGGGATCAAGAAGGGAGAATGAACTATTACTGGACACTAGTAGAG | |
| | CCGGGAGACAAAATAACATTCGAAGCAACTGGAAATCTAGTGGTACC | |
| | GAGATATGCATTCGCAATGGAAAGAAATGCTGGATCTGGTATTATCAT | |
| | TTCAGATACACCAGTCCACGATTGCAATACAACTTGTCAGACACCCAA | |
| | GGGTGCTATAAACACCAGCCTCCCATTTCAGAACATACATCCGATCA | |
| | CAATTGGAAAATGTCCAAAATATGTAAAAAGCACAAAATTGAGACTGG | |
| | CCACAGGATTGAGGAATGTCCCGTCCATTCAATCTAGAGGCCTATTT | |
| | GGGGCCATTGCCGGTTTCATTGAAGGGGGGTGGACAGGGATGGTAG | |
| | ATGGATGGTACGGTTATCACCATCAAAATGAGCAGGGGTCAGGATAT | |
| | GCAGCCGACCTGAAGAGCACACAGAATGCCATTGACGAGATTACTAA | |
| | CAAAGTAAACTCTGTTATTGAAAAAATGAATACACAGTTCACAGCAGT | |
| | AGGTAAAGAGTTCAACCACCTGGAAAAAAGAATAGAGAATTTAAATAA | |
| | AAAAGTTGATGATGGTTTCCTGGACATTTGGACTTACAATGCCGAACT | |
| | GTTGGTTCTATTGGAAAATGAAAGAACTTTGGACTACCACGATTCAAA | |
| | GGTGAAGAACTTATATGAAAAGGTAAGAAGCCAGTTAAAAAACAATG | |
| | CCAAGGAAATTGGAAACGGCTGCTTTGAATTTTACCACAAATGTGATA | |
| | ACACGTGCATGGAAAGTGTCAAAAATGGGACTTATGACTACCCAAAA | |
| | TACTCAGAGGAAGCAAAATTAAACAGAGAAGAAATAGATGGGGTAAA | |
| | GCTGGAATCAACAAGGATTTACCAGATTTTGGCGATCTATTCAACCGT | |
| | CGCCAGTTCATTGGTACTGGTAGTCTCCCTGGGGGCAATCAGTTTCT | |
| | GGATGTGCTCTAATGGGTCTCTACAGTGTAGAATATGTATTTAATGAT | |
| | GA | |
| Pan-CoV-Flu | ATGAGCAAAAGCAGGGGATAATTCTATTAACCATGAAGACTATCATTG | 420 |
| Vaccine #32 | CTTTGAGCTACATTCTATGTCTGGTTTTCGCTCAAAAACTTCCTGGAA | |
| (H3N2) | ATGACAATAGCACTGCAACGCTGTGCCTTGGGCACCATGCAGTACCA | |
| | AACGGAACGATAGTGAAAACAATCACGAATGACCAAATTGAAGTTACT | |
| | AATGCTACTGAGCTGGTTCAGAATTCCTCAATAGGTGAAATATGCGA | |
| | CAGTCCTCATCAGATCCTTGATGGAGAAAACTGCACACTAATAGATG | |
| | CTCTATTGGGAGACCCTCAGTGTGATGGCTTTCAAAATAAGAAATGG | |
| | GACCTTTTTGTTGAACGAAGCAAAGCCTACAGCAACTGTTACCCTTAT | |
| | GATGTGCCGGATTATGCCTCCCTTAGGTCACTAGTTGCCTCATCCGG | |
| | CACACTGGAGTTTAACAATGAAAGCTTCAATTGGGCTGGAGTCACTC | |
| | AAAACGGAACAAGTTCTGCTTGCATAAGGGGATCTAATAGTAGTTTCT | |
| | TTAGTAGATTAAATTGGTTGACCCACTTAAACTTCAAGTACCCAGCAT | |
| | TGAACGTGACTATGCCAAACAATGAACAATTTGACAAATTGTACATTT | |
| | GGGGGGTTCACCACCCGGGTACGGACAAGGACCAAATCTTCCTGTA | |
| | TGCTCAATCATCAGGAAGAATCACAGTATCTACCAAAAGAAGCCAAC | |
| | AAGCTGTAATCCCGAATATCGGATCTAGACCCAGAATAAGGAATATC | |
| | CCTAGCAGAATAAGCATCTATTGGACAATAGTAAAACCGGGAGACAT | |
| | ACTTTTGATTAACAGCACAGGGAATCTAATTGCTCCTAGGGGTTACTT | |
| | CAAAATACGAAGTGGGAAAAGCTCAATAATGAGATCAGATGCACCCA | |
| | TTGGCAAATGCAAGTCTGAATGCATCACTCCAAATGGAAGCATTCCC | |
| | AATGACAAACCATTCCAAAATGTAAACAGGATCACATACGGGGCCTG | |
| | TCCCAGATATGTTAAGCAAAGCACTCTGAAATTGGCAACAGGAATGC | |
| | GAAATGTACCAGAGAAACAAACTAGAGGCATATTTGGCGCAATAGCG | |
| | GGTTTCATAGAAAATGGTTGGGAGGGAATGGTGGATGGTTGGTACG | |
| | GTTTCAGGCATCAAAATTCTGAGGGAAGAGGACAAGCAGCAGATCTC | |
| | AAAAGCACTCAAGCAGCAATCGATCAAATCAATGGGAAGCTGAATCG | |
| | ATTGATCGGGAAAACCAACGAGAAATTCCATCAGATTGAGAAAGAATT | |
| | CTCAGAAGTAGAAGGGAGAATTCAGGACCTTGAGAAATATGTTGAGG | |
| | ACACAAAAATAGATCTCTGGTCATACAACGCAGAGCTTCTTGTTGCCC | |
| | TGGAAAACCAACATACAATTGATCTAACTGACTCAGAAATGAACAAAC | |
| | TGTTTGAAAAAACAAAGAAGCAACTGAGGGAAAATGCTGAGGATATG | |
| | GGCAATGGTTGTTTCAAAATATACCACAAATGTGACAATGCCTGCATA | |
| | GGATCAATCAGAAATGGAACTTATGACCACGATGTATACAGGGATGA | |
| | AGCATTAAACAACCGGTTCCAGATCAAGGGAGTTGAGCTGAAGTCAG | |
| | GGTACAAAGATTGGATCCTATGGATTTCCTTTGCCATATCATGTTTTTT | |
| | GCTTTGTGTTGCTTTGTTGGGGTTCATCATGTGGGCCTGCCAAAAGG | |
| | GCAACATTAGGTGCAACATTTGCATTTGAGTGCATTAATTAAAAACAC | |
| | CCTTGTTTCTACTTGATGA | |
| Pan-CoV-Flu | ATGATTTTCTAATATCCACAAAATGAAGGCAATAATTGTACTACTCATG | 421 |
| Vaccine #33 | GTAGTAACATCCAATGCAGATCGAATCTGCACTGGGATAACATCGTC | |
| (H3N2) | AAACTCACCACATGTCGTCAAAACTGCTACTCAAGGGGAGGTCAACG | |
| | TGACCGGTGTAATACCACTGACAACAACACCCACCAAATCTCATTTTG | |
| | CAAATCTCAAAGGAACAGAAACCAGGGGGAAACTATGCCCAAAATGC | |
| | CTCAACTGCACAGATCTGGATGTAGCCTTGGGCAGACCAAAATGCAC | |
| | AGGGAAAATACCCTCTGCAAGGGTTTCAATACTCCATGAAGTCAGAC | |
| | CTGTTACATCTGGGTGCTTTCCTATAATGCACGATAGAACAAAAATTA | |
| | GACAGCTGCCTAACCTTCTCCGAGGATACGAACATGTCAGGTTATCA | |
| | ACTCACAACGTTATCAATGCAAAAGATGCACCAGGAAGACCCTACGA | |
| | AATTGGAACCTCAGGGTCTTGCCCTAACATTACCAATGGAAACGGAT | |
| | TCTTCGCAACAATGGCTTGGGCCGTCCCAAAAAACAAAACAGCAACA | |
| | AATCCATTAACAATAGAAGTACCATACATTTGTACAGAAGGAGAAGAC | |
| | CAAATTACCGTTTGGGGGTTCCACTCTGACAACGAGACCCAAATGGC | |
| | AAAGCTCTATGGGGACTCAAAGCCCCAGAAGTTCACCTCATCTGCCA | |
| | ACGGAGTGACCACACATTACGTTTCACAGATTGGTGGCTTCCCAAAT | |
| | CAAACAGAAGACGGAGGACTACCACAAAGTGGCAGAATTGTTGTTGA | |
| | TTACATGGTGCAGAAATCTGGAAAAACAGGAACAATTACCTATCAAAG | |
| | AGGTATTTTATTGCCTCAAAAAGTGTGGTGCGCAAGTGGCAGGAGCA | |
| | AGGTAATAAAAGGATCCTTGCCCTTAATTGGAGAAGCAGATTGCCTC | |
| | CATGAAAAATACGGTGGATTAAACAAAAGCAAGCCTTACTACACAGG | |
| | GGAACATGCAAAGGCCATAGGAAATTGCCCAATATGGGTGAAAACAC | |
| | CCTTGAAGCTGGCCAATGGAACCAAATATAGACCCCCTGCAAAACTA | |
| | TTAAAGGAAAGAGGTTTCTTCGGAGCCATTGCTGGTTTCTTAGAGGG | |
| | AGGATGGGAAGGAATGATTGCAGGTTGGCACGGATACACATCCCAT | |
| | GGGGCACATGGAGTAGCGGTGGCAGCTGACCTTAAGAGCACTCAAG | |
| | AGGCCATAAACAAGATAACAAAAAATCTCAACTCTTTGAGTGAGCTGG | |
| | AAGTAAAGAATCTTCAAAGACTAAGCGGTGCCATGGATGAACTCCAC | |
| | AACGAAATACTAGAACTAGATGAGAAAGTGGATGATCTCAGAGCTGA | |
| | CACAATAAGCTCACAAATAGAACTCGCAGTCCTGCTTTCCAATGAAG | |
| | GAATAATAAACAGTGAAGATGAACATCTCTTGGCGCTTGAAAGAAAG | |
| | CTGAAGAAAATGCTGGGCCCCTCTGCTGTAGAGATAGGGAATGGAT | |
| | GCTTTGAAACCAAACACAAGTGCAACCAGACCTGCCTCGACAGAATA | |
| | GCTGCTGGTACCTTTGATGCAGGAGAATTTTCTCTCCCCACCTTTGAT | |
| | TCACTGAATATTACTGCTGCATCTTTAAATGACGACGGATTGGACAAT | |
| | CATACTATACTGCTTTACTACTCAACTGCTGCCTCCAGTTTGGCTGTA | |
| | ACACTGATGATAGCTATCTTTGTTGTTTATATGGTCTCCAGAGACAAT | |
| | GTTTCTTGCTCCATTTGTCTATAAGGAAAGTTAAGCCCTGTATTTTCCT | |
| | TTATTGTAGTGCTTGTTTGCTTGTTGTCATTACAAAGAAACGTTATTGA | |
| | AAAATTGATGA | |

In some embodiments, the recombinant vaccine compositions described herein are used to prevent a coronavirus and/or influenza disease in a subject. In other embodiments, the recombinant vaccine compositions described herein are used to prevent a coronavirus and/or influenza infection prophylactically in a subject. In further embodiments, the recombinant vaccine compositions described herein elicits an immune response in a subject.

The present invention also features oligonucleotide compositions. For example, the present invention includes oligonucleotides disclosed in the sequence listings. The present invention also includes oligonucleotides in the form of antigen delivery systems. The present invention also includes oligonucleotides encoding the conserved large sequences disclosed herein. The present invention also includes oligonucleotide compositions comprising one or more oligonucleotides encoding any of the vaccine compositions according to the present invention. In some embodiments, the oligonucleotide comprises DNA. In some embodiments, the oligonucleotide comprises modified DNA. In some embodiments, the oligonucleotide comprises RNA. In some embodiments, the oligonucleotide comprises modified RNA. In some embodiments, the oligonucleotide comprises mRNA. In some embodiments, the oligonucleotide comprises modified mRNA.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

What is claimed:

1. A universal pre-emptive pan-Coronavirus vaccine composition, the composition comprising one or more large sequences, wherein the one or more large sequences comprise at least one of:
a) one or more conserved coronavirus B-cell target epitopes;
b) one or more conserved coronavirus CD4⁺ T cell target epitopes; and/or
c) one or more conserved coronavirus CD8⁺ T cell target epitopes;
wherein at least one epitope is derived from a non-spike protein.

2. The composition of claim 1, wherein the non-spike proteins are encoded by ORF1ab, ORF3a, ORF6, ORF7a, ORF7b, ORF8, or ORF10, or derived from an Envelope protein, a Membrane protein, or a Nucleocapsid protein.

3. The composition of claim 1, wherein at least one large sequence is highly conserved among human and animal Coronaviruses.

4. The composition of claim 1, wherein the one large sequence is derived from one or more of: one or more SARS-CoV-2 human strains or variants in current circulation; one or more SARS-CoV-2 variants identified in the future; one or more coronaviruses that has caused a previous human outbreak; one or more coronaviruses isolated from animals selected from a group consisting of bats, pangolins, civet cats, minks, camels, and other animal receptive to coronaviruses; or one or more coronaviruses that cause the common cold, past, current, and future coronavirus outbreaks.

5. The composition of claim 4, wherein the one or more SARS-CoV-2 human strains or variants in current circulation are selected from: variant B.1.177; variant B.1.160, variant B.1.1.7 (UK), variant P.1 (Japan/Brazil), variant B.1.351 (South Africa), variant B.1.427 (California), variant B.1.429 (California), variant B.1.258; variant B.1.221; variant B.1.367; variant B.1.1.277; variant B.1.1.302; variant B.1.525; variant B.1.526, variant S:677H; variant S:677P; B.1.617.2-Delta, variant B.1.1.529-Omicron (BA.1); sub-variant Omicron (BA.1); sub-variant Omicron (BA.2); sub-variant Omicron (BA.3); sub-variant Omicron (BA.4); sub-variant Omicron (BA.5).

6. The composition of claim 4, wherein the one or more coronaviruses that cause the common cold are selected from: 229E alpha coronavirus, NL63 alpha coronavirus, OC43 beta coronavirus, and HKU1 beta coronavirus.

7. The composition of claim 1, wherein at least one large sequence is a whole protein sequence expressed by SARS-CoV-2, a partial protein sequence expressed by SARS-CoV-2, or a combination thereof.

8. The composition of claim 7, wherein the whole protein sequence or the partial protein sequence expressed by SAR-CoV-2 is selected from SEQ ID NO: 182-193, SEQ ID NO: 148-159, SEQ ID NO: 360-383, and SEQ ID NO: 348-359.

9. The composition of claim 1 further comprising a T cell attracting chemokine, wherein the T cell attracting chemokine is CCL5, CXCL9, CXCL10, CXCL11, or a combination thereof.

10. The composition of claim 1 further comprising a composition that promotes T cell proliferation and T-cell memory, wherein the composition promotes T cell proliferation and memory is IL-7, IL-2, or IL-15.

11. The composition of claim 1, wherein the composition comprises any of SEQ ID NO: 139-147 and SEQ ID NO: 295-347.

12. The composition of claim 1, wherein the vaccine composition protects against disease caused by one or more coronavirus variants or coronavirus subvariants.

13. The composition of claim 12, wherein the coronavirus variants or coronavirus subvariants comprise past or currently circulating coronavirus variants or coronavirus subvariants wherein the coronavirus variants comprise alpha, beta, gamma, delta, and omicron.

14. The composition of claim 12, wherein the coronavirus variants or coronavirus subvariants comprise future variants or future subvariants of human and animal coronavirus.

15. The composition of claim 1, wherein the vaccine composition protects against infection and re-infection of coronavirus variants or coronavirus subvariants.

16. The composition of claim 15, wherein the coronavirus variants or coronavirus subvariants comprise past or currently circulating coronavirus variants or coronavirus subvariants, wherein the coronavirus variants comprise alpha, beta, gamma, delta, and omicron.

17. The composition of claim 15, wherein the coronavirus variants or coronavirus subvariants comprise future variants or future subvariants of human and animal coronavirus.

18. The composition of claim 15, wherein the vaccine composition protects against infection or reinfection of one or more coronavirus variants or coronavirus subvariant.

19. The composition of claim 18, wherein the vaccine composition protects against infection or reinfection of multiple coronavirus variants or coronavirus subvariants.

20. The composition of claim 18, wherein the vaccine composition protects against infection or re-infection of one coronavirus variants or coronavirus subvariants.

21. The composition of claim 1, wherein the vaccine composition induces strong and long-lasting protection mediated by antibodies (Abs), CD4+ T helper (Th1) cells, and/or CD8+ cytotoxic T-cells.

22. The composition of claim 1, wherein the composition protects against Sarbecoviruses, wherein sarbecoviruses comprise SARS-CoV1 or SARS-CoV2.
